(19) United States
(12) Patent Application Publication
(10) Pub. No.: US 2026/0297080 A1
(43) Pub. Date: Oct. 1, 2026

(54) BICYCLIC COMPOUNDS AS CDK INHIBITORS

(71) Applicant: BeOne Medicines I GmbH, Basel (CH)

(72) Inventors: Changxin HUO, Beijing (CN); Yu ZHAO, Beijing (CN); Weixing ZHANG, Beijing (CN); Huaqing LIU, Beijing (CN); Zhiwei WANG, Beijing (CN)

(73) Assignee: BeOne Medicines I GmbH, Basel (CH)

(21) Appl. No.: 19/077,474

(22) Filed: Mar. 12, 2025

Related U.S. Application Data
(63) Continuation of application No. PCT/CN2023/118754, filed on Sep. 14, 2023.

(30) Foreign Application Priority Data
Sep. 15, 2022 (WO) PCT/CN2022/118948
Apr. 6, 2023 (WO) PCT/CN2023/086678
Jul. 20, 2023 (WO) PCT/CN2023/108474
Aug. 16, 2023 (WO) PCT/CN2023/113327

(51) Int. Cl. C07D 417/12; A61K 31/4155; A61K 31/428; A61K 31/433; A61K 31/437; A61K 31/454; A61K 31/5377; A61K 31/5415; C07D 409/12; C07D 417/14; C07D 513/04

(52) U.S. Cl. CPC C07D 417/12; A61K 31/4155; A61K 31/428; A61K 31/433; A61K 31/437; A61K 31/454; A61K 31/5377; A61K 31/5415; C07D 409/12; C07D 417/14; C07D 513/04

(57) ABSTRACT

Disclosed herein are bicyclic compound derivatives used as inhibitors of cyclin-dependent kinases. Disclosed herein is the use of these inhibitors for inhibiting cyclin-dependent kinases, and the use of such compounds for treating cancer.

### CROSS-REFERENCE TO RELATED APPLICATIONS

This application is a continuation of International Application No. PCT/CN2023/118754, filed on Sep. 14, 2023, which claims priority to International Application No. PCT/CN2023/113327, filed on Aug. 16, 2023, International Application No. PCT/CN2023/108474, filed on Jul. 20, 2023, International Application No. PCT/CN2023/086678, filed on Apr. 6, 2023, and International Application No. PCT/CN2022/118948, filed on Sep. 15, 2022, the disclosures of which are hereby incorporated by reference in their entireties.

### FIELD OF THE INVENTION

Disclosed herein are bicyclic compound derivatives used as inhibitors of cyclin-dependent kinases. Disclosed herein is the use of these inhibitors for inhibiting cyclin-dependent kinases, and the use of such compounds for treating cancer.

### BACKGROUND OF THE INVENTION

Cyclin-dependent kinases (CDKs), a family of Ser/Thr protein kinases, function as a driver of the cell cycle (Norbury, C. and Nurse, P., Annu Rev Biochem, 61, 441-470). During the cell cycle, CDK4 and CDK6 initiate the retinoblastoma (Rb) phosphorylation by binding to cyclinD and partially release the transcription factor E2F. Then the CDK2 forms a complex with cyclinE to fully phosphorylate Rb, entirely release E2F and initiate S-phase (Harbour, J. W. et al., Cell, 98 (6), 859-869). Then in the S phase, CDK2 forms a complex with cyclinA (Echalier, A. et al., Biochim Biophys Acta, 1804 (3), 511-519). Interestingly, in the normal cells, CDK2 is mostly not essential for the cell cycle, while in some cancer cells, CDK2 kinase activity plays a critical role in the abnormal growth process (Caruso, J. A. et al., Cancer Res, 78 (19), 5481-5491).

CCNE gene amplification or cyclinE overexpressed forms of cancer cells over activates CDK2, dysregulating Rb phosphorylation and resulting in cancer cell proliferation (Wood, D. J. et al., Cell Chem Biol, 26 (1), 121-130). Aberrant CCNE has been proved as a disease driver in multiple cancer types such as ovarian, esophageal, bladder, pancreatic and so on, which are associated with poor disease outcomes (Au-Yeung, G. et al., Clin Cancer Res, 23 (7), 1862-1874; DeLair, D. F. et al., J Pathol, 243 (2), 230-241; Fu, Y. P. et al., Cancer Res, 74 (20), 5808-5818; Huber, A. R. et al., BMC Gastroenterol, 15, 80; Miller, C. T. et al., Clin Cancer Res, 9 (13), 4819-4825). Besides, one of the mechanisms for the clinical drug resistance of CDK4/6i (such as Palbociclib, Ribociclib and Abemaciclib) in ER+HER2-breast cancer patients is the overexpression of CCNE and the activation of CDK2 (Knudsen, E. S. et al., Cell Rep, 38 (9), 110448). CDK2 siRNA knockdown in the Palbociclib resistant breast cancer cell lines and mouse model shows CDK2 inhibition can overcome the CDK4/6-inhibitor resistance (Pandey, K. et al., Cancers (Basel), 12 (12)). Another potential use of CDK2 inhibitors is based on the mechanism of trastuzumab resistance. There is around 35% trastuzumab resistance incidence of cyclinE amplification or overexpression in HER2+BC patients, which are associated with a worse clinical benefit while the trastuzumab-resistant cells are sensitive to CDK2 inhibition (Scaltriti, M. et al., Proc Natl Acad Sci USA, 108 (9), 3761-3766). The CCNE1 amplification across broad cancer types predicts response to CDK2 inhibition, indicating CDK2 is a potentially impactful therapeutic target.

The low toxicity of targeting CDK2 has also been proved in the animal model. CDK2 knockout mice are viable and developed normally except for the abnormal germline cell (Berthet, C. et al., Curr Biol, 13 (20), 1775-1785). While single knockout of CDK1 or double knockout of CDK4 and CDK6 mice are embryonic lethal (Satyanarayana, A. and Kaldis, P., Oncogene, 28 (33), 2925-2939).

The crystal structure of CDK2 especially its kinase domain is highly similar to the CDK1 and the commercial CDK2 inhibitors are mostly with poor CDK1 selectivity (Wells, C. I. et al., Nat Commun, 11 (1), 2743). It is desirable to achieve a cancerous specific drug so as to derive a high selective CDK2 inhibitor, sparing CDK family members to limit off-target CDK-driven toxicities, especially CDK1/CDK4/CDK6.

### SUMMARY OF THE INVENTION

In one embodiment, disclosed herein are bicyclic compound derivatives of Formula (I). The embodiment comprises the following aspects:

Aspect 1. A compound of Formula (I):

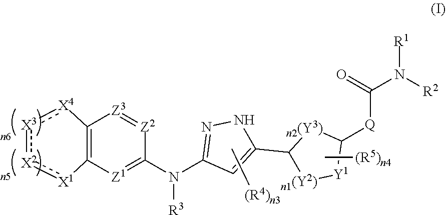

or a N-oxide thereof, or a pharmaceutically acceptable salt thereof, or a stereoisomer thereof, or a tautomer, or a deuterated analog thereof, or a prodrug thereof, wherein:
n1 is 0, 1, or 2;
n2 is 1, 2 or 3;
n3 is 0, 1, or 2;
n4 is 0, 1, 2, 3 or 4;
n5 and n6 are each independently 0 or 1, provided that n5 and n6 are not 0 at the same time;
each of [symbol] is independently a single bond or double bond, provided that two double bonds are not connected directly;
X¹, X², X³ and X⁴ are each independently selected from N, C, S(═O)₂ or S(═O), wherein each Nis independently substituted with 0 or 1 R^{Xa} groups as allowed by valence, and each C is independently substituted with one or two R^{Xb} groups as allowed by valence;
R^{Xa} is each independently selected from hydrogen, —C₁-C₈alkyl, —C₂-C₈alkenyl, —C₂-C₈alkynyl, —C₃-C₈cycloalkyl, 3- to 12-membered heterocyclyl, —C₆-C₁₂aryl or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{Xaa};
R^{Xaa} is each independently selected from hydrogen, deuterium, halogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl, 5- to 12-membered heteroaryl, oxo (═O), —OR^{Xab}, —CN, —SO₂R^{Xab}, —SO₂NR^{Xab}R^{Xac}, —C(O)R^{Xab}, —CO₂R^{Xab}, —C(O)NR^{Xab}R^{Xac}, —NR^{Xab}R^{Xac}, —NR^{Xab}COR^{Xac}, —NR^{Xab}CO₂R^{Xac} or -NR^{Xab}SO₂R^{Xac}, wherein each of said —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{Xad}, —SO₂R^{Xad}, —SO₂NR^{Xad}R^{Xae}, —C(O)R^{Xad}, —CO₂R^{Xad}, —C(O)NR^{Xad}R^{Xae}, —NR^{Xad}R^{Xae}, NR^{Xad}COR^{Xae}, —NR^{Xad}CO₂R^{Xae} or —NR^{Xad}SO₂R^{Xae};
R^{Xab}, R^{Xac}, R^{Xad} and R^{Xae} are each independently selected from hydrogen, deuterium, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with halogen, —C₁-C₈alkyl, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —CN, —OH, or —C₁-C₈alkoxyl;
R^{Xb} is each independently selected from hydrogen, halogen, —C₁-C₈alkyl, C₃-C₈cycloalkyl, 3- to 8-membered heterocyclyl, C₆-C₁₂aryl, 5- to 12-membered heteroaryl, oxo (═O), —NR^{Xba}R^{Xbb}, —OR^{Xba}—SR^{Xba}, —SO₂R^{Xba}, —SO₂NR^{Xba}R^{Xbb}, —C(O)R^{Xba}, —CO₂R^{Xba}, —C(O)NR^{Xba}R^{Xbb}, —NR^{Xba}COR^{Xbb}, —NR^{Xba}CO₂R^{Xbb} or —NR^{Xba}SO₂R^{Xbb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Xbc}, or two R^{Xb} together with the atom(s) to which they are attached, form a 3- to 12-membered ring, said ring comprising 0-3 heteroatoms independently selected from nitrogen, oxygen or optionally oxidized sulfur as ring member(s); said ring is optionally substituted with at least one substituent R^{Xbc}.
R^{Xba} and R^{Xbb} are each independently selected from hydrogen, deuterium, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Xbd}.
R^{Xbc} and R^{Xbd} are each independently selected from hydrogen, deuterium, halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Xbe}R^{Xbf}, —OR^{Xbe}, —SR^{Xbe}, —SO₂R^{Xbe}, —SO₂NR^{Xbe}R^{Xbf}, —C(O)R^{Xbe}, —CO₂R^{Xbe}, —C(O)NR^{Xbe}R^{Xbf}, —NR^{Xbe}COR^{Xbf}, —NR^{Xbe}CO₂R^{Xbf} or —NR^{Xbe}SO₂R^{Xbf} or —CN;
R^{Xbe} and R^{Xbf} are each independently selected from C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
Y¹, Y² and Y³ are each independently selected from O, C or N, wherein C and N are independently substituted with 1 or 2 R⁵ groups or hydrogen atoms as allowed by valence;
Q is selected from O or NR^{Q};
R¹, R², R³ and R^{Q} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{1a}; or
(R¹ and R²), (R¹ and R^{Q}) or (R^{Q} and R²) together with the atom(s) to which they are attached, form a 3- to 12-membered ring, said ring comprising 0-3 additional heteroatoms independently selected from nitrogen, oxygen or optionally oxidized sulfur as ring member(s); said ring is optionally substituted with at least one substituent R^{1b};
R^{1a} and R^{1b} are each independently selected from hydrogen, halogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl, 5- to 12-membered heteroaryl, oxo (═O), —OR^{1c}, —CN, —SO₂R^{1c}, —SO₂NR^{1c}R^{1d}, —C(O)R^{1c}, —CO₂R^{1c}, —C(O)NR^{1c}R^{1d}, —NR^{1c}R^{1d}, —NR^{1c}COR^{1d}, —NR^{1c}CO₂R^{1d} or —NR^{1c}SO₂R^{1d}, wherein each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{1e}, —SO₂R^{1e}, —SO₂NR^{1e}R^{1f}, —C(O)R^{1e}, —CO₂R^{1f}, —C(O)NR^{1e}R^{1f}, —NR^{1e}R^{1f}, —NR^{1e}COR^{1f}, —NR^{1e}CO₂R^{1f} or —NR^{1a}SO₂R^{1f};
R^{1c}, R^{1d}, R^{1e} and R^{1f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁴ and R⁵ are each independently selected from hydrogen, halogen, —C₁-C₈alkyl, —C₂-C₈alkenyl, —C₂-C₈alkynyl, —C₁-C₈alkoxy, or —C₃-C₈cycloalkyl;
Z¹, Z² and Z³ are each independently selected from —CR^{Z}, or N;
R^{Z}, at each occurrence, is independently selected from hydrogen, halogen, —C₁-C₈alkyl, C₃-C₈cycloalkyl, 3- to 8-membered heterocyclyl, C₆-C₁₂aryl, 5- to 12-membered heteroaryl, —NR^{Za}R^{Zb}, —OR^{Za}, —SR^{Za}, —SO₂R^{Za}, —SO₂NR^{Za}R^{Za}, —C(O)R^{Za}, —CO₂R^{Za}, —C(O)NR^{Za}R^{Zb}, —NR^{Za}COR^{Zb}, —NR^{Za}CO₂R^{Zb} or -NR^{Za}SO₂R²b or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc}.
R^{Za} and R^{Zb} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Zd}.
R^{Zc} and R^{Zd} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Ze}R^{Zf}, —OR^{Ze}, —SR^{Ze}, —SO₂R^{Ze}, —SO₂NR^{Ze}R^{Zf}, —C(O)R^{Ze}, —CO₂R^{Ze}, —C(O)NR^{Ze}R^{Zf}, —NR^{Ze}COR^{Zf}, —NR^{Ze}CO₂R^{Zf} or —NR^{Ze}SO₂R^{Zf} or —CN;
R^{ze} and R^{Zf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl.

In some embodiments, at least one of X¹, X², X³ and X⁴ is N, at least one of X¹, X², X³ and X⁴ is S(═O)₂ or S(═O).

In some embodiments, at most one of Y¹, Y² and Y³ is selected from O or N, and the other two of Y¹, Y² and Y³ are both C.

Aspect 2. The compound of Aspect 1, wherein the compound is selected from (IIa), (IIb), (IIc) and (IId):

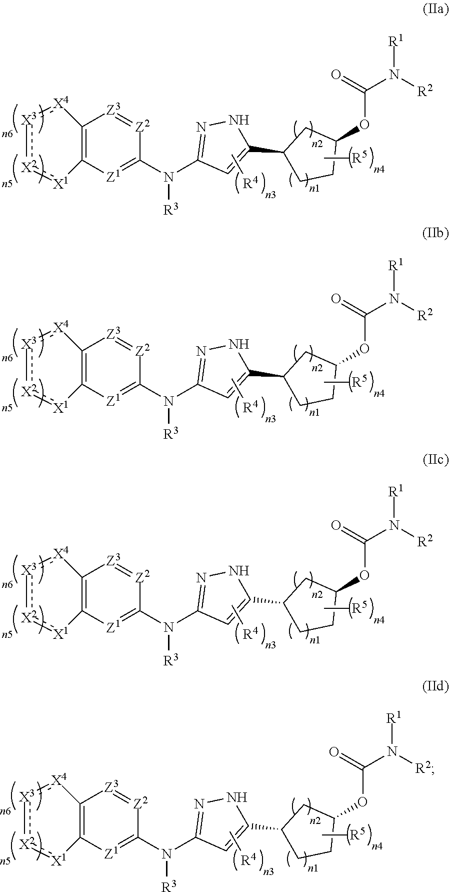

preferably, the compound is selected from (Ile), (IIf), (IIg), (IIh), (IIi) and (IIj):

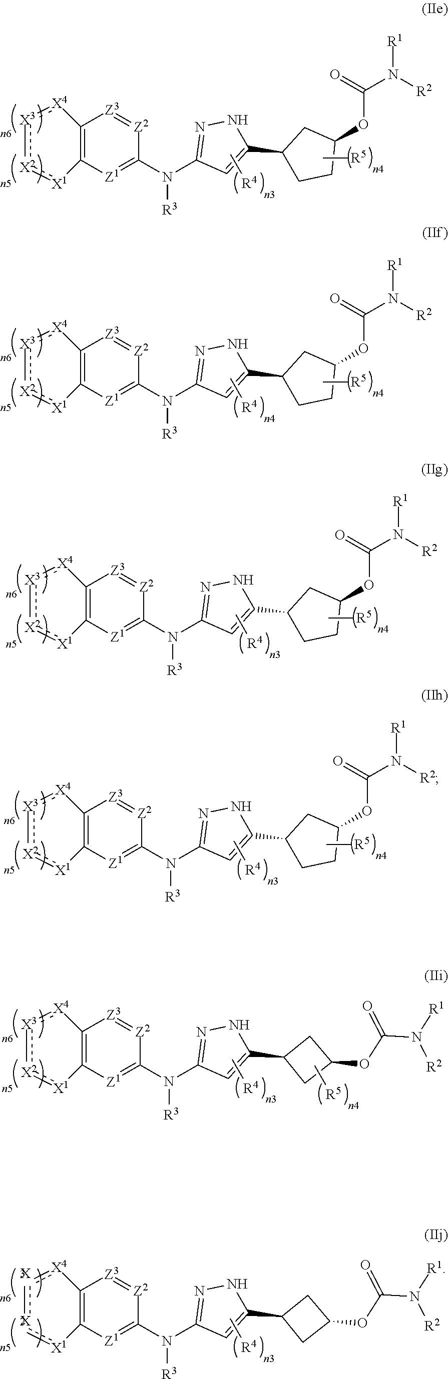

Aspect 3. The compound of Aspect 1, wherein the compound is selected from (IIIa), (IIIb), (IIIc), (IIId), (IIIe), (IIIf), (IIIg) and (IIIh):

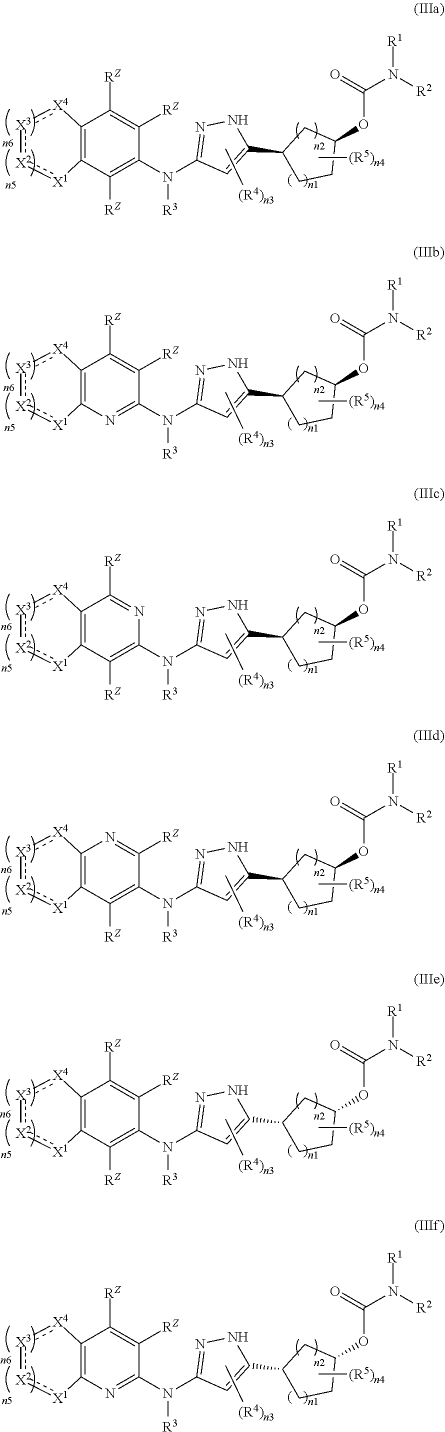

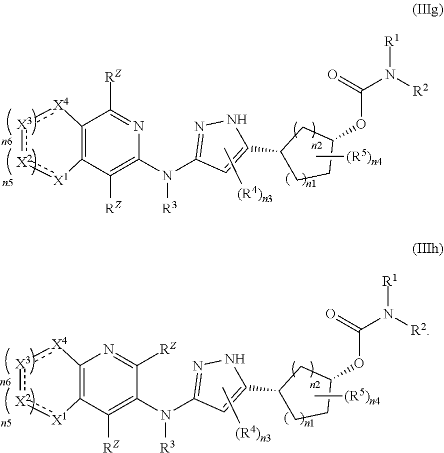

Aspect 4. The compound of Aspect 1, wherein the compound is selected from (IVa), (IVb), (IVc), (IVd), (IVe), (IVf), (IVg), (IVh), (IVi), (IVj), (IVk), (IVI), (IVm), (IVn), (IVo), (IVp), (IVq), (IVr), (IVs), (IVt), (IVu), (IVv), (IVw) and (IVx):

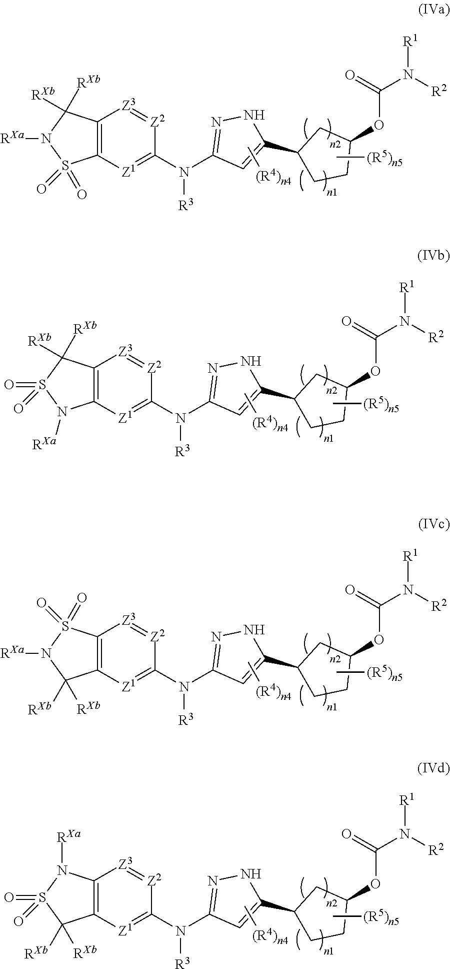

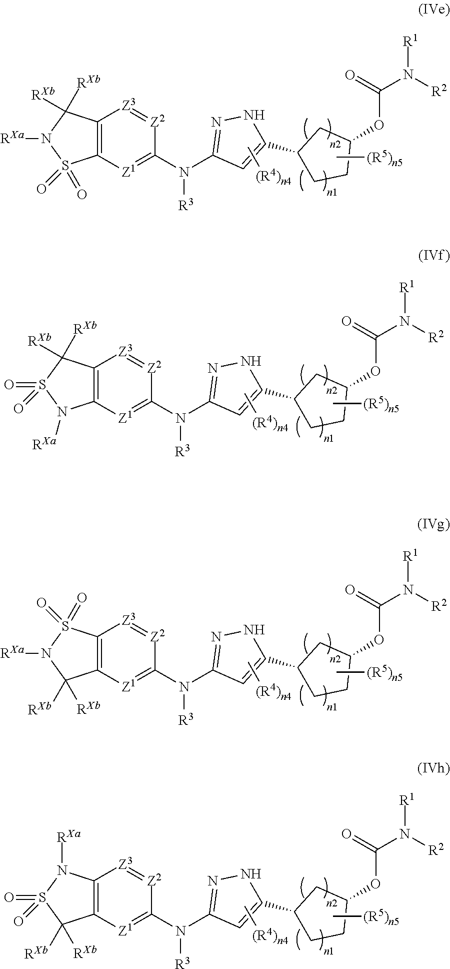

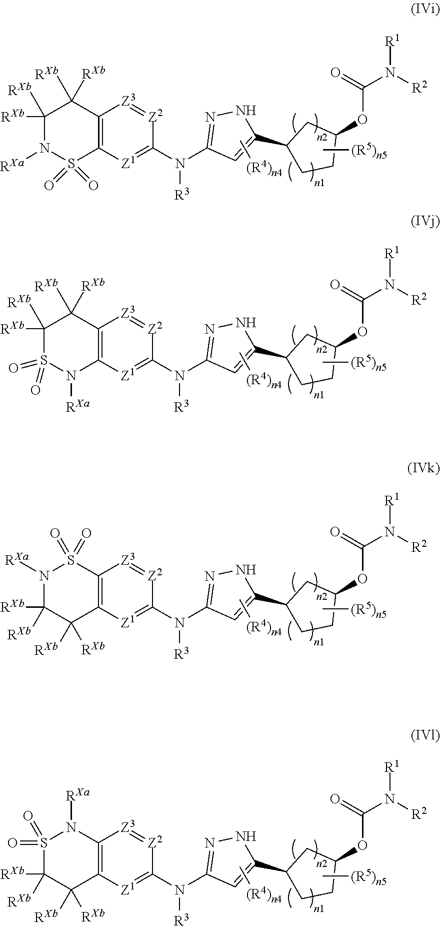

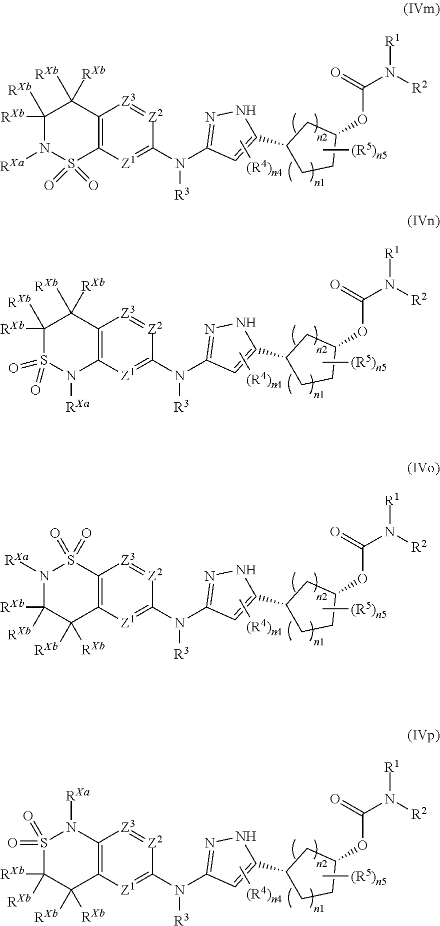

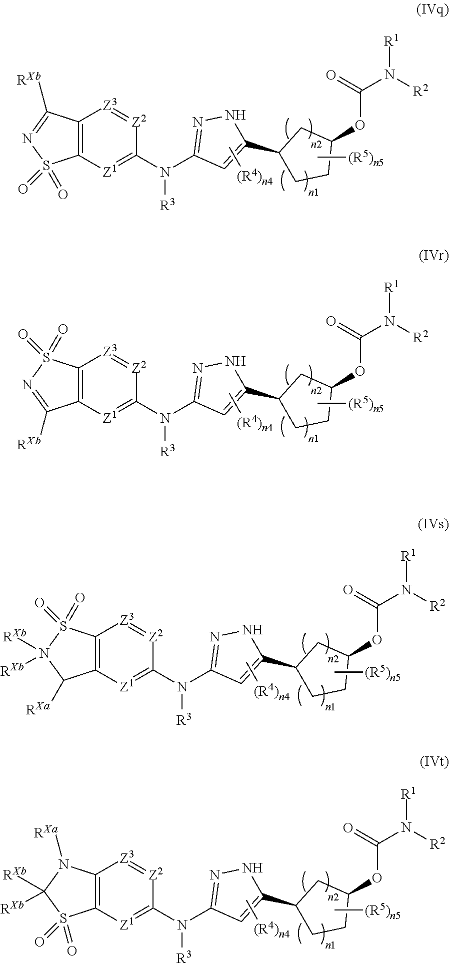

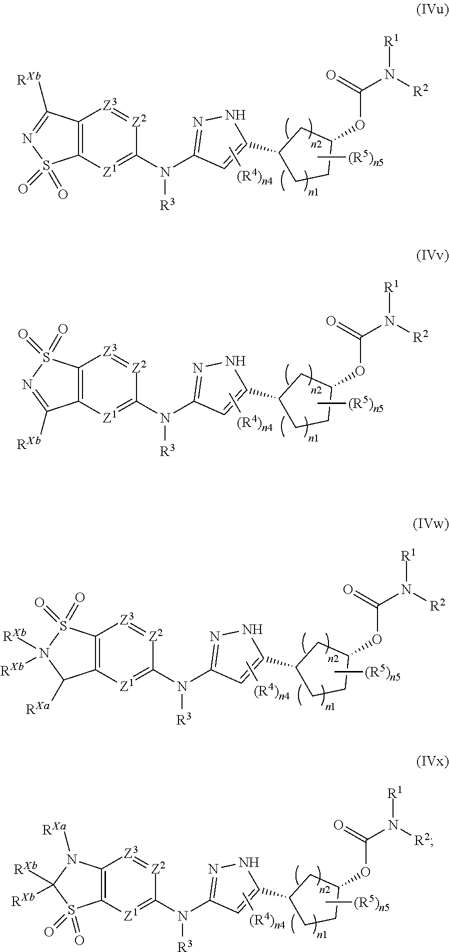

preferably, the compound is selected from (Va) and (VIa):

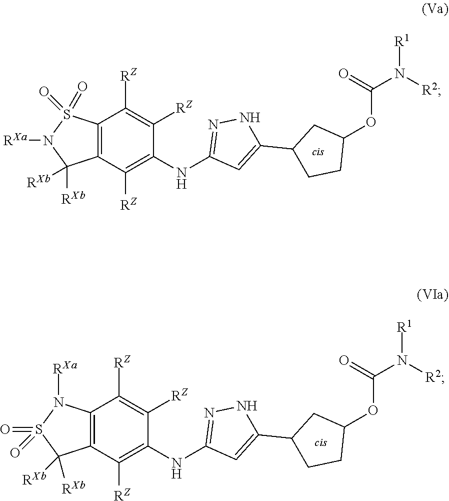

preferably, the compound is selected from (Vb), (Vc), (VIb) and (VIc):

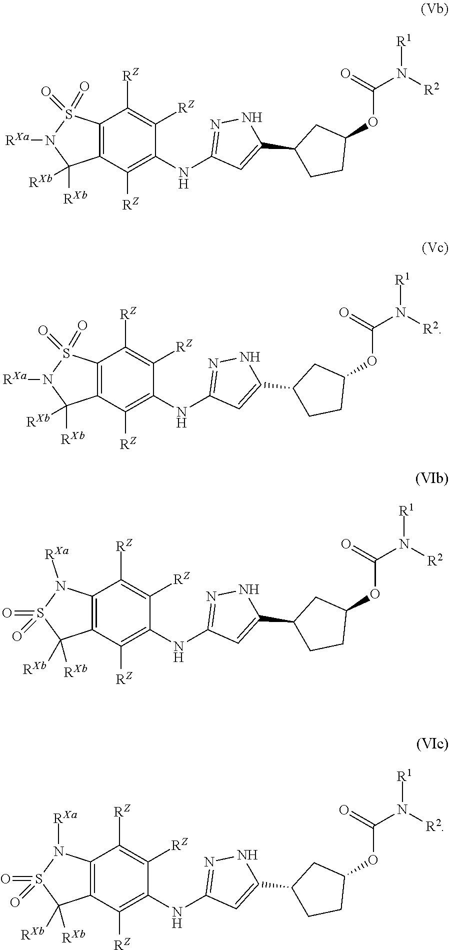

more preferably, the compound is selected from (Vd) and (VId)

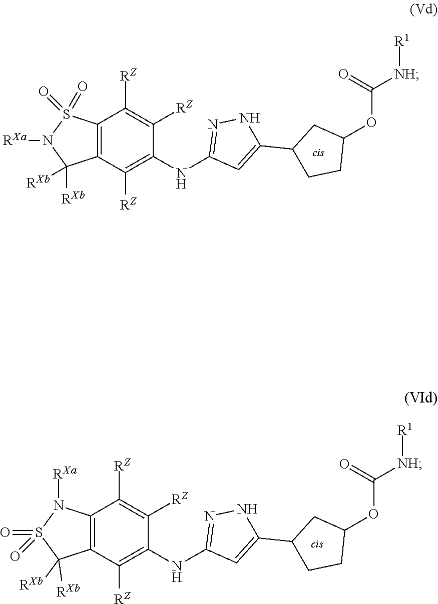

even more preferably, the compound is selected from (Ve), (Vf), (VIe) and (VIf):

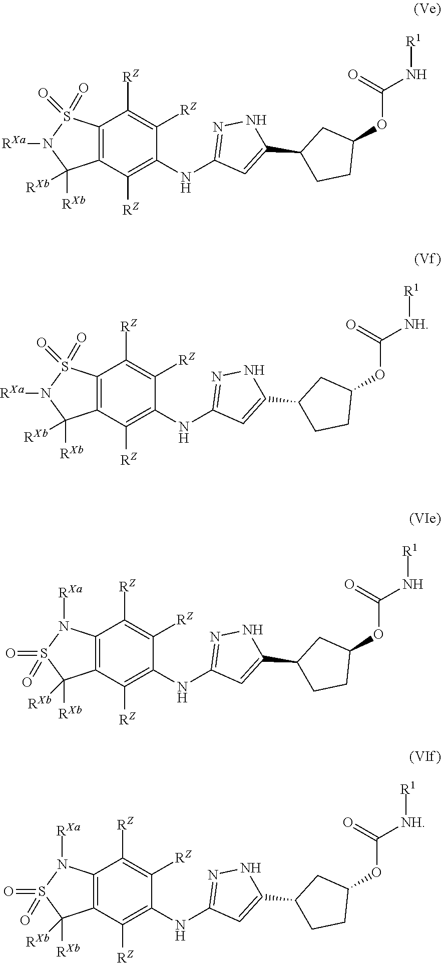

Aspect 5. The compound of anyone of the preceding aspects, wherein R¹, R², R³ and Re are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, cyclononyl, cyclodecyl, 3- to 8-membered heterocyclyl, phenyl or 5- to 12-membered heteroaryl is optionally substituted with at least one substituent R^{1a};
R^{1a} is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{1c}, —CN, —SO₂R^{1c}, —SO₂NR^{1c}R^{1d}, —C(O)R^{1c}, —CO₂R^{1c}, —C(O)NR^{1c}R^{1d}, —NR^{1c}CR^{1d}, —NR^{1c}COR^{1d}, —NR^{1c}CO₂R^{1d} or —NR^{1c}SO₂R^{1d}, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{1e}, —SO₂R^{1e}, —SO₂NR^{1e}R^{1f}, —C(O)R^{1e}, —CO₂R^{1f}, —C(O)NR^{1e}R If, -NR^{1e}ER^{1f}, —NR^{1e}COR^{1f}, —NR^{1e}CO₂R^{1f} or —NR^{1e}SO₂R^{1f},
R^{1c}, R^{1d}, R^{1e} and R^{1f} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy;
preferably, R¹, R², R³ and R° are each independently selected from hydrogen, methyl, ethyl, propyl(n-propyl or isopropyl), butyl(n-butyl, sec-butyl, iso-butyl or tert-butyl), pentyl, hexyl, heptyl, octyl, -C₂-C₈alkenyl, —C₂-C₈alkynyl, cyclopropyl, cyclobutyl, cyclopentyl, cyclohexyl, cycloheptyl, cyclooctyl, cyclononyl, cyclodecyl, 3- to 8-membered heterocyclyl, phenyl or 5- to 12-membered heteroaryl;
more preferably, R¹, R², R³ and R^{Q} are each independently selected from hydrogen, methyl, ethyl, propyl(n-propyl or isopropyl), butyl(n-butyl, sec-butyl, iso-butyl or tert-butyl);
even more preferably, R¹ and R² are each independently selected from hydrogen, methyl, ethyl, propyl(n-propyl or isopropyl), butyl(n-butyl, sec-butyl

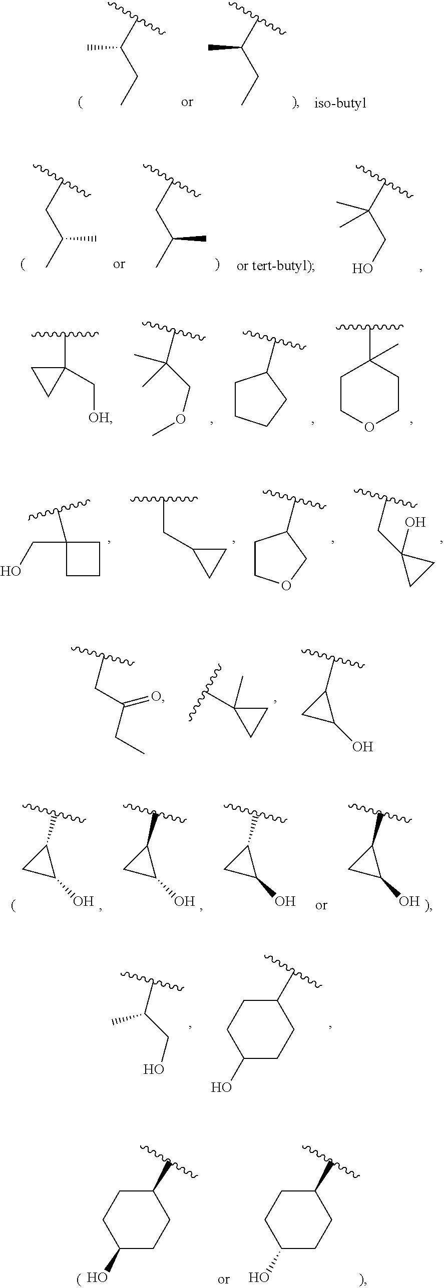

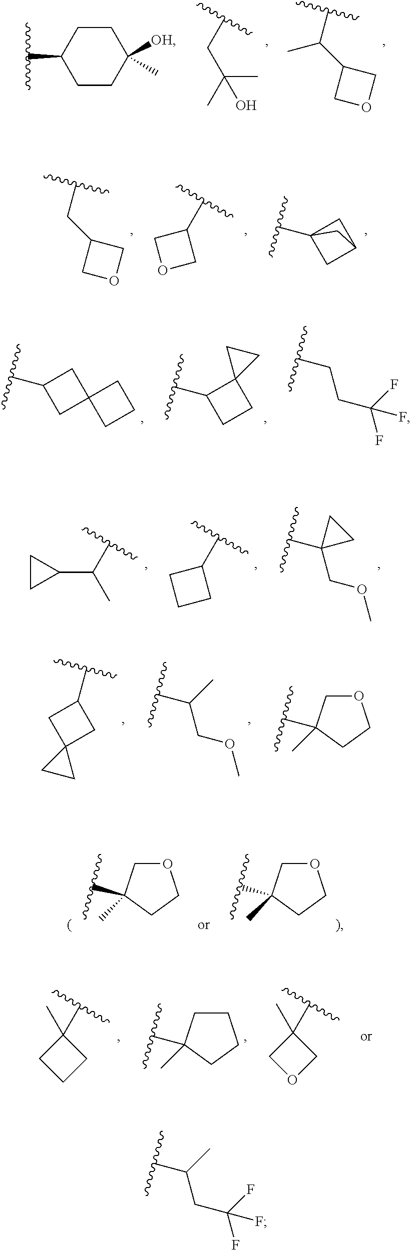

and R³ and R^{Q} are each independently hydrogen.

In another embodiment, R¹ and R² are independently substituted or unsubstituted bridged, spiro or fused cyclic groups, for example, bicyclo[2.2.1]heptyl, bicyclo[2.2.2]octyl, bicyclo[2.1.1]hexyl, or bicyclo[1.1.1]pentyl.

Aspect 6. The compound of anyone of the preceding aspects, wherein R¹ is independently selected from hydrogen, methyl, ethyl, propyl(n-propyl or isopropyl), butyl(n-butyl, sec-butyl, iso-butyl or tert-butyl), pentyl, hexyl, cyclopropyl, cyclobutyl, cyclopentyl, cyclohexyl, azetidinyl, oxetanyl, oxanyl, bicyclo[1.1.1]pentanyl, spiro[3.3]heptanyl, spiro[2.3]hexanyl or tetrahydrofuranyl, wherein each of said methyl, ethyl, propyl(n-propyl or isopropyl), butyl(n-butyl, sec-butyl, iso-butyl or tert-butyl), pentyl, hexyl, cyclopropyl, cyclobutyl, cyclopentyl, cyclohexyl, azetidinyl, oxetanyl, oxanyl, bicyclo[1.1.1]pentanyl, spiro[3.3]heptanyl, spiro[2.3]hexanyl or tetrahydrofuranyl is optionally substituted with at least one substituent R^{1a};
R^{1a} is independently selected from hydrogen, —F, —Cl, —Br, —I, oxo (—O), methyl, ethyl, propyl(n-propyl or isopropyl), cyclopropyl, cyclobutyl, cyclopentyl, cyclohexyl, azetidinyl, oxetanyl, bicyclo[1.1.1]pentanyl, spiro[3.3]heptanyl, spiro[2.3]hexanyl, tetrahydrofuranyl or —OR^{1c}; wherein each of said methyl, ethyl, propyl(n-propyl or isopropyl), cyclopropyl, cyclobutyl, cyclopentyl, cyclohexyl is optionally substituted with OR^{1c};
R¹c is independently selected from hydrogen, methyl, ethyl, propyl(n-propyl or isopropyl), butyl(n-butyl, sec-butyl, iso-butyl or tert-butyl), cyclopropyl, cyclobutyl; and R² is hydrogen;
preferably, R¹ is independently selected from hydrogen, methyl, ethyl, propyl(n-propyl or isopropyl), butyl(n-butyl, sec-butyl, iso-butyl or tert-butyl), pentyl, hexyl, cyclopropyl, cyclobutyl, cyclopentyl, cyclohexyl, azetidinyl, oxetanyl, oxanyl, bicyclo[1.1.1]pentanyl, spiro[3.3]heptanyl, spiro[2.3]hexanyl or tetrahydrofuranyl, wherein each of said methyl, ethyl, propyl(n-propyl or isopropyl), butyl(n-butyl, sec-butyl, iso-butyl or tert-butyl), pentyl, hexyl, cyclopropyl, cyclobutyl, cyclopentyl, cyclohexyl, azetidinyl, oxetanyl, oxanyl, bicyclo[1.1.1]pentanyl, spiro[3.3]heptanyl, spiro[2.3]hexanyl or tetrahydrofuranyl is optionally substituted with at least one substituent selected from OH, CH₂OH, CH₂OMe, F, Cl, Br, oxo (═O), OMe, OEt, Methyl, Ethyl, cyclopropyl, cyclopropyl-OH, cyclobutyl, azetidinyl, oxetanyl, bicyclo[1.1.1]pentanyl, spiro[3.3]heptanyl, spiro[2.3]hexanyl or tetrahydrofuranyl; and R² is hydrogen;
more preferably, R¹ is independently selected from methyl, ethyl, propyl(n-propyl or isopropyl), butyl(n-butyl, sec-butyl

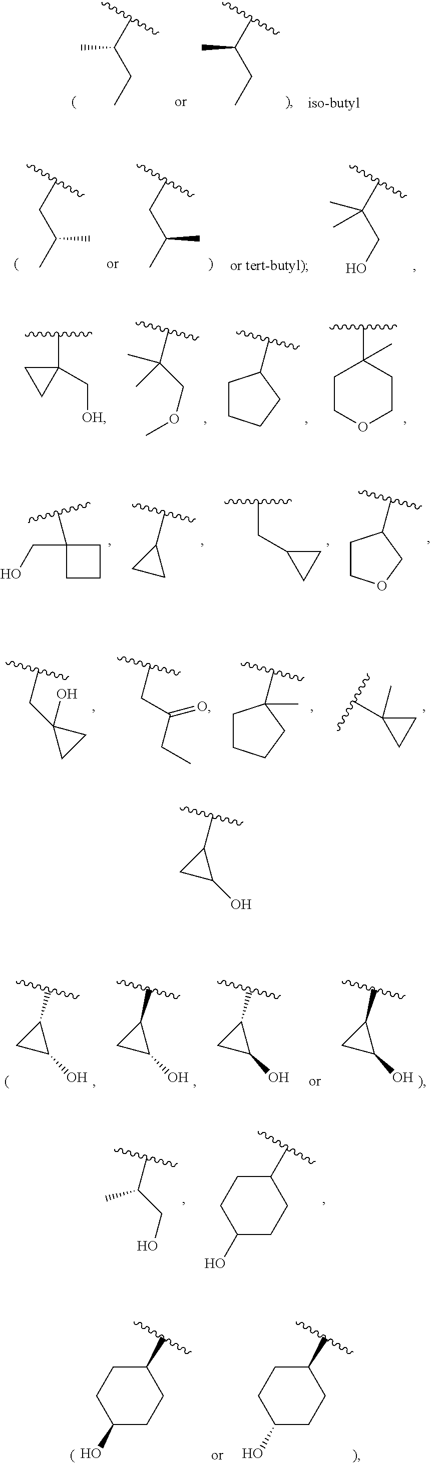

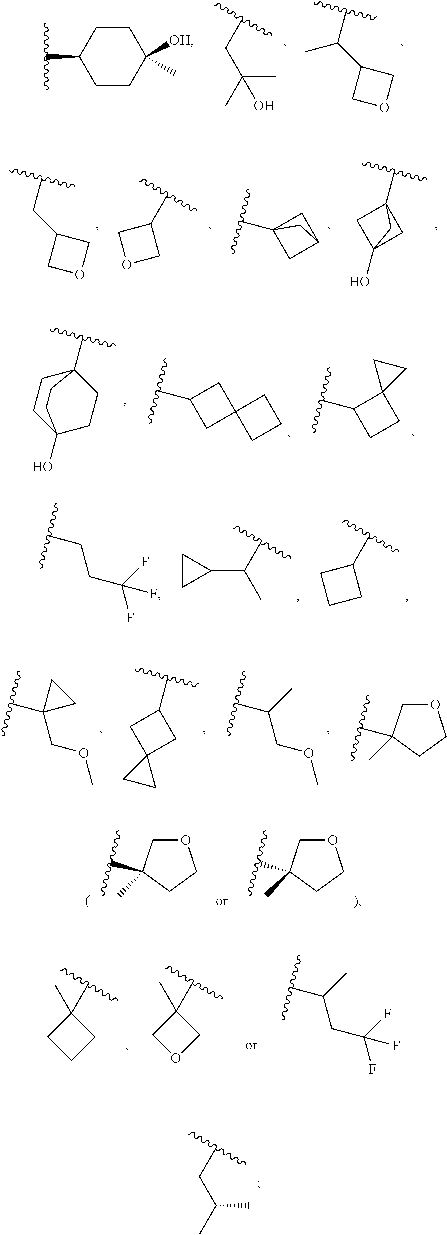

and R² is hydrogen.

Aspect 7. The compound of anyone of the preceding aspects, wherein (R¹ and R²), (R¹ and R^{Q}) or (R° and R²) together with the atom(s) to which they are attached, form a 3-, 4-, 5-, 6-, 7- or 8-membered ring, said ring comprising 0, 1, 2 or 3 additional heteroatoms independently selected from nitrogen, oxygen or optionally oxidized sulfur as ring member(s); said ring is optionally substituted with at least one substituent R^{1b};
R^{1b} is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —OR^{1c}, —CN, —SO₂R^{1c}, —SO₂NR^{1c}R^{1d}, —C(O)R^{1c}, —CO₂R^{1c}, —C(O)NR^{1c}R^{1d}, —NR^{1c}CR^{1d}, —NR^{1c}COR^{1d}, —NR^{1c}CO₂R^{1d} or —NR^{1c}SO₂R^{1d}, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{1e}, —SO₂R^{1e}, —SO₂NR^{1e}R^{1f}, —C(O)R^{1e}, -CO₂R¹f, —C(O)NR^{1e}R^{1f}, —NR^{1e}R^{1f}, —NR^{1e}COR^{1f}, —NR^{1e}CO₂R^{1f} or —NR^{1e}SO₂R^{1f};
R^{1c}, R^{1d}, R^{1e} and R^{1f} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy;
preferably, (R¹ and R²), (R¹ and R^{Q}) or (RR and R²) together with the atom(s) to which they are attached, form a 4-,5- or 6-membered ring; said ring is optionally substituted with at least one substituent R^{1b};
R^{1b} is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —OR^{1c}, —CN, —SO₂R^{1c}, —SO₂NR^{1c}R^{1d}, —C(O)R^{1c}, —CO₂R^{1c}, —C(O)NR^{1c}R^{1d}, —NR^{1c}R^{1d}, —NR^{1c}COR^{1d}, —NR^{1c}CO₂R^{1d} or —NR^{1c}SO₂R^{1d};
R^{1c} and R^{1d} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy;
more preferably, (R¹ and R²), (R¹ and R^{Q}) or (R^{Q} and R²) together with the atom(s) to which they are attached, form a 4-,5- or 6-membered ring; said ring is optionally substituted with at least one substituent R^{1b};
R^{1b} is independently selected from hydrogen, —F, —Cl, —Br, —I, methyl, ethyl, propyl(n-propyl or isopropyl), butyl(n-butyl, sec-butyl, iso-butyl or tert-butyl), cyclopropyl, cyclobutyl, cyclopentyl, cyclohexyl, —OH, methoxy, ethoxy, propoxy, butoxy, pentoxy, hexoxy, hepthoxy, octoxy, —CN, or —NH₂;
even more preferably, R¹ and R² together with the nitrogen atom to which they are attached, form a 4- or 5-membered ring; said ring is optionally substituted with at least one substituent R^{1b};
R^{1b} is independently selected from hydrogen, —F, —Cl, methyl, ethyl, propyl(n-propyl or isopropyl), butyl(n-butyl, sec-butyl, iso-butyl or tert-butyl) or —OH.

Aspect 8. The compound of anyone of the preceding aspects, wherein the

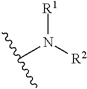

moiety is selected from

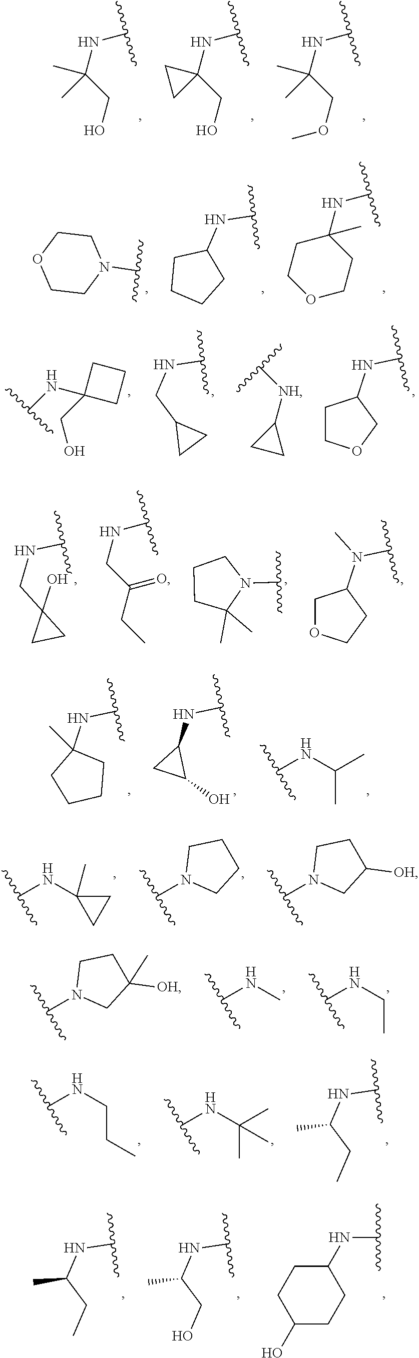

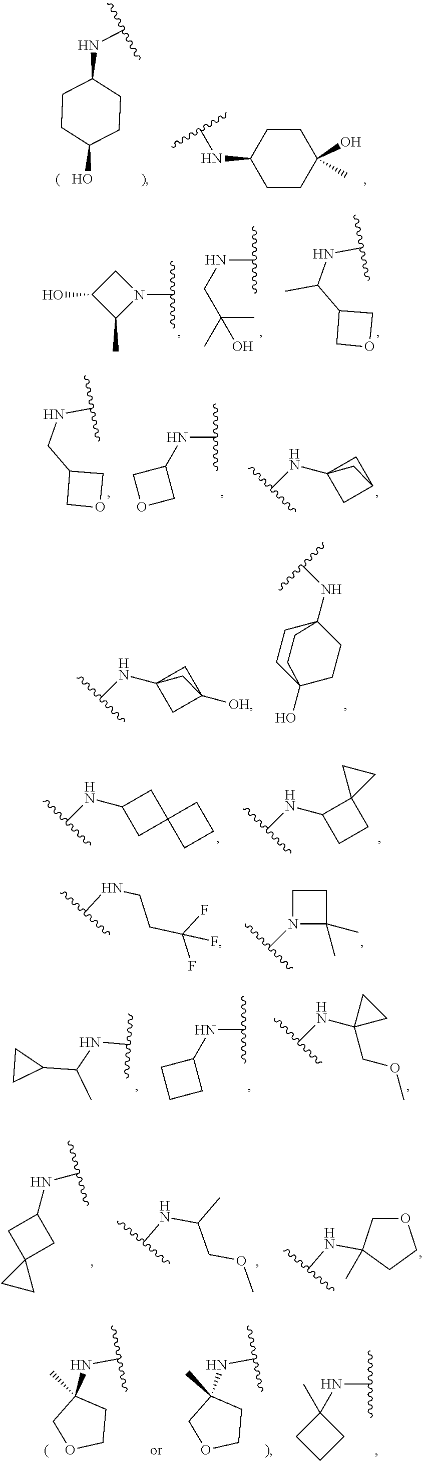

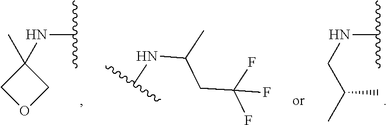

Aspect 9. The compound of anyone of the preceding aspects, wherein R⁴ and R⁵ are each independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl;
preferably, R⁴ and R⁵ are each independently selected from hydrogen, —F, —Cl, —Br, —I, methyl, ethyl, propyl(n-propyl or isopropyl), butyl(n-butyl, sec-butyl, iso-butyl or tert-butyl), methoxy, ethoxy, propoxy, cyclopropyl, cyclobutyl, cyclopentyl;
even more preferably, R⁴ and R⁵ are each independently selected from hydrogen.

Aspect 10. The compound of anyone of the preceding aspects, wherein the

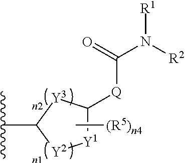

moiety is selected from

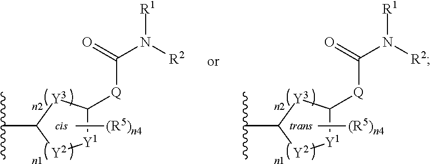

preferably, the

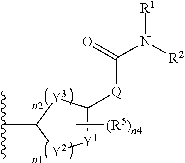

moiety is selected from

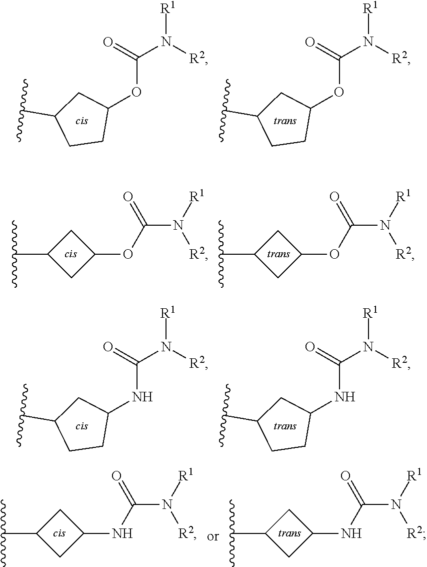

more preferably, the

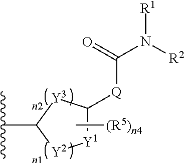

moiety is selected from

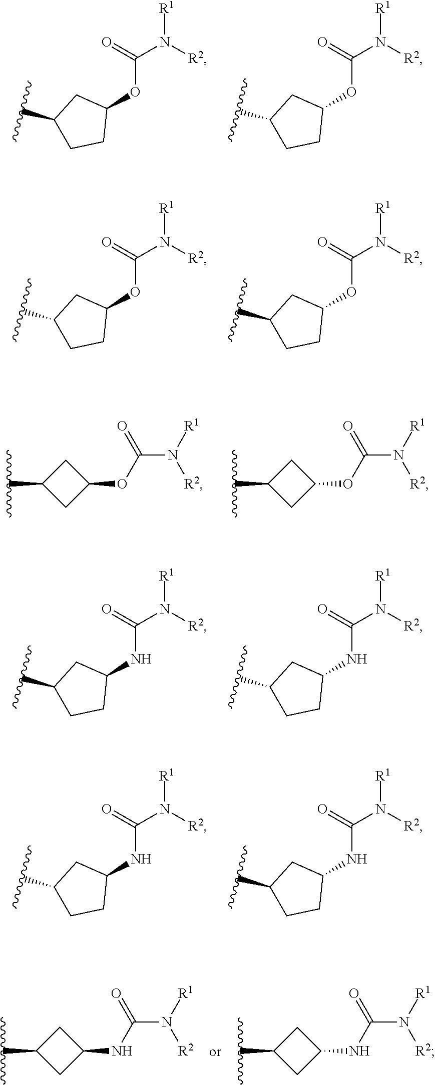

even more preferably, the

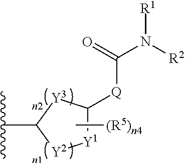

moiety is selected from

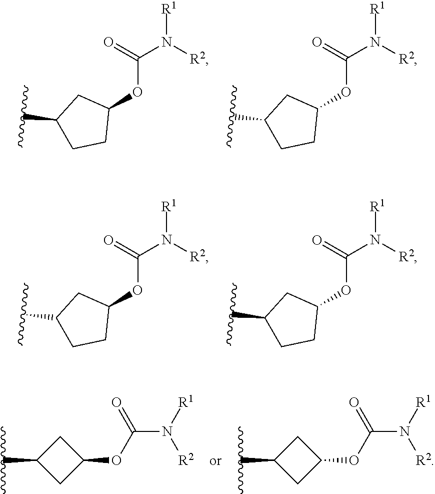

Aspect 11. The compound of anyone of the preceding aspects, wherein at most two of Z¹, Z² and Z³ are N; preferably, at most one of Z¹, Z² and Z³ is N;
more preferably, Z¹ is N and Z² and Z³ are —CR^{Z}; or Z² is N and Z¹ and Z³ are —CR^{Z}; or Z³ is N and Z¹ and Z² are —CR^{Z}.

Aspect 12. The compound of anyone of the preceding aspects, wherein R^{Z}, at each occurrence, is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Za}, —NR^{Za}CO₂R^{2b} or —NR^{Za}SO₂R^{Zb} or —CN, wherein each of said methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{Zc};
R^{Za} and R^{Zb} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{Zd};
R^{Zc} and R^{Zd} are each independently selected from —F, —Cl, —Br, —I, hydroxy, methyl, ethyl, propyl(n-propyl or isopropyl), butyl(n-butyl, sec-butyl, iso-butyl or tert-butyl), pentyl, hexyl, heptyl, octyl, methoxy, ethoxy, propoxy, butoxy, pentoxy, hexoxy, hepthoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{Ze}R^{Zf}, —OR^{Ze}, —SR^{Ze}, —SO₂R^{Ze}, —SO₂NR^{Ze}R^{Zf}, —C(O)R^{ze}, —CO₂R^{ze}, —C(O)NR^{Ze}R^{Zf}, —NR^{Ze}COR^{Zf}, —NR^{Ze}CO₂R^{Zf} or —NR^{Ze}SO₂R^{Zf} or —CN;
R^{ze} and R^{zf} are each independently selected from methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, R^{Z}, at each occurrence, is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{Za}SO₂R^{2b} or —CN;
R^{Za} and R^{2b} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R^{Z}, at each occurrence, is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NH₂, —OH, methoxy, ethoxy, propoxy, butoxy, pentoxy, hexoxy, hepthoxy, octoxy or —CN;
even more preferably, R^{Z}, at each occurrence, is independently selected from hydrogen, —F, —Cl, methyl, ethyl, propyl(n-propyl or isopropyl), butyl(n-butyl, sec-butyl, iso-butyl or tert-butyl), cyclopropyl, cyclobutyl, cyclopentyl, cyclohexyl, —CH₂F, —CHF₂, —CF₃, —CH₂CH₂F, —CH₂OH, —CH₂CHF₂, —CH₂CF₃, —NH₂, —OH, —OCHF₂, methoxy, ethoxy, propoxy, or butoxy.

Aspect 13. The compound of anyone of the preceding aspects, wherein the

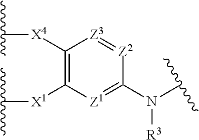

moiety is selected from

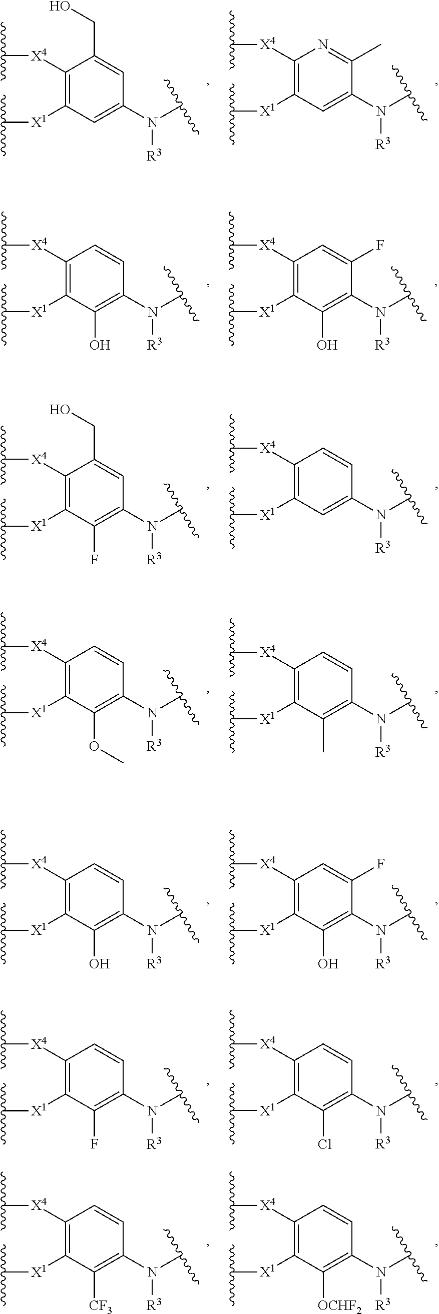

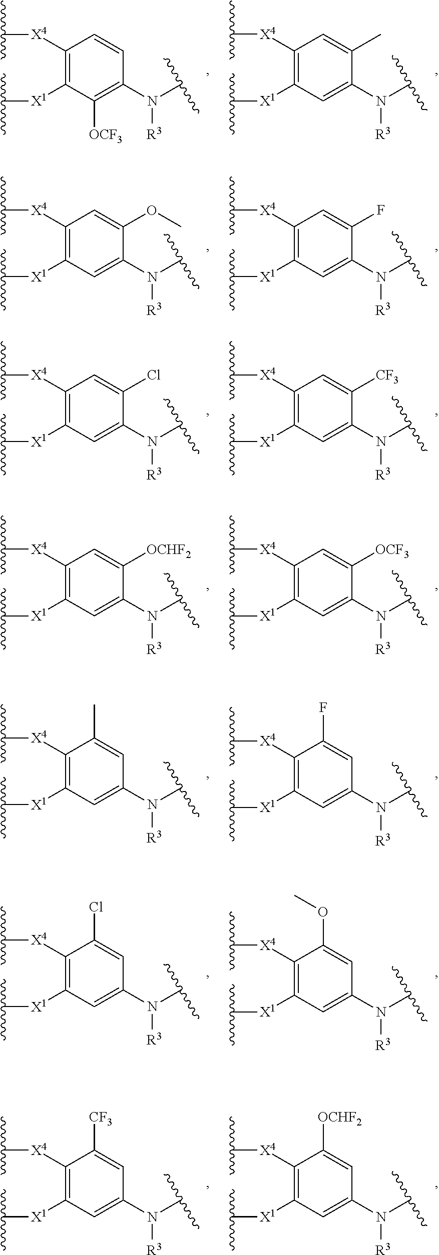

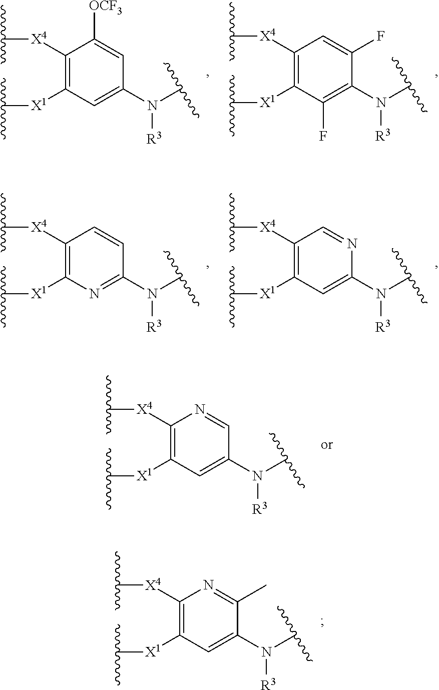

preferably, the

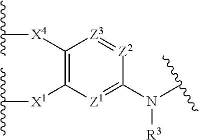

moiety is selected from

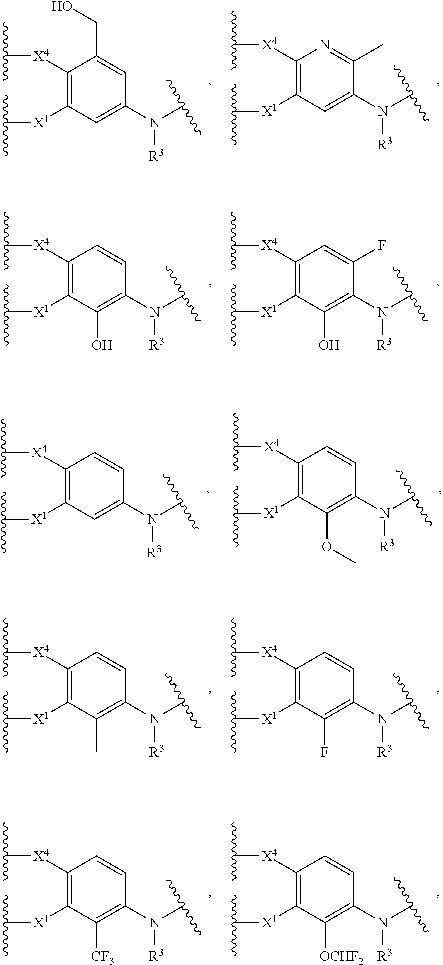

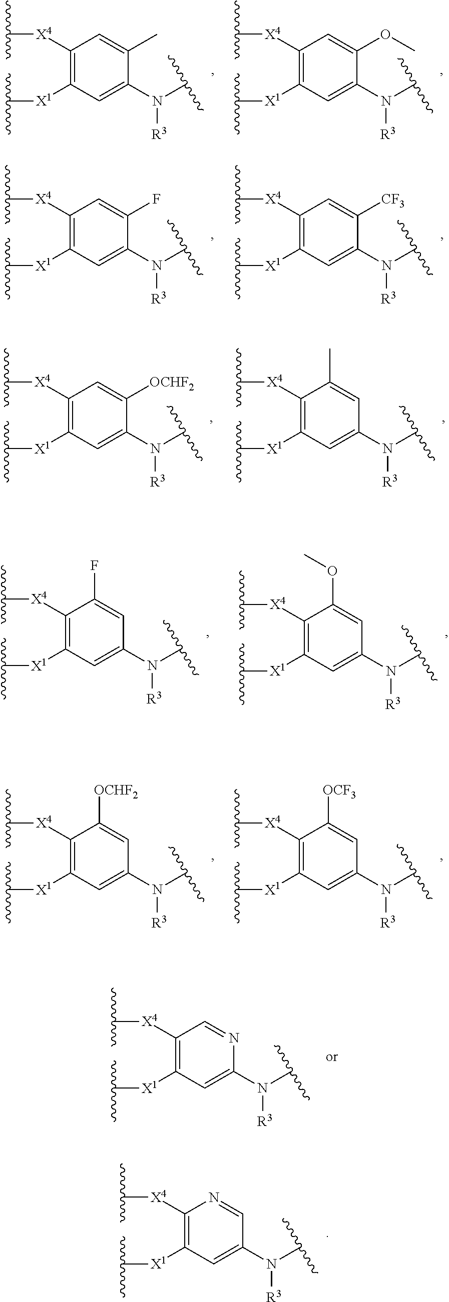

Aspect 14. The compound of anyone of the preceding aspects, wherein X¹, X², X³ and X⁴ are each independently selected from N, NR^{Xa}, CR^{Xb}, C(R^{Xb})₂, S(═O)₂, provided that X¹, X², X³ and X⁴ are allowed by valence; preferably, at most one of X¹, X², X³ and X⁴ is selected from N or NR^{Xa}, at most one of X¹, X², X³ and X⁴ is selected from S(═O) 2 or S(═O).

Aspect 15. The compound of anyone of the preceding aspects, wherein R^{Xa} is each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Xaa};
R^{Xaa} is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{Xab}, —CN, —SO₂R^{Xab}, —SO₂NR^{Xab}R^{Xac}, —C(O)R^{Xab}, —CO₂R^{Xab}, —C(O)NR^{Xab}R^{Xac}, —NR^{Xab}R^{Xac}, —NR^{Xab}COR^{Xac}, —NR^{Xab}CO₂R^{Xac} or —NR^{Xab}SO₂R^{Xac}, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{Xad}, —SO₂R^{Xad}, —SO₂NR^{Xad}R^{Xae}, —C(O)R^{Xad}, —CO₂R^{Xad}, —C(O)NR^{Xad}R^{Xae}, —NR^{Xad}R^{Xae}, —NR^{Xad}COR^{Xae}, —NR^{Xad}CO₂R^{Xae} or —NR^{Xad}SO₂R^{Xe};
R^{Xab} R^{Xac}, R^{Xad} and R^{Xae} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy;
preferably, R^{Xa} is each independently selected from hydrogen, methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or 3- to 8-membered heterocyclyl, wherein each of said methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one substituent R^{Xaa};
R^{Xaa} is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{Xab}, —CN, —C(O)R^{Xab} or —NR^{Xab}R^{Xac}, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{Xad}, —SO₂R^{Xad}, —C(O)R^{Xad} or —NR^{Xad}R^{Xae};
R^{Xab}, R^{Xac}, R^{Xad} and R^{Xae} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy;
more preferably, R^{Xa} is each independently selected from hydrogen, methyl, ethyl, propyl(n-propyl or isopropyl), butyl(n-butyl, sec-butyl, iso-butyl or tert-butyl), pentyl, cyclopropyl, cyclobutyl, cyclopentyl, cyclohexyl, piperidinyl, tetrahydrofuranyl, azetidinyl or pyrrolidinyl, wherein each of said methyl, ethyl, propyl(n-propyl or isopropyl), butyl(n-butyl, sec-butyl, iso-butyl or tert-butyl), pentyl, cyclopropyl, cyclobutyl, cyclopentyl, cyclohexyl, piperidinyl, tetrahydrofuranyl, azetidinyl, oxetanyl or pyrrolidinyl is optionally substituted with at least one substituent R^{Xaa};
R^{Xaa} is independently selected from hydrogen, —F, —Cl, —Br, methyl, ethyl, propyl(n-propyl or isopropyl), butyl(n-butyl, sec-butyl, iso-butyl or tert-butyl), cyclopropyl, cyclobutyl, cyclopentyl, cyclohexyl, oxo (═O), —OH, methoxy, ethoxy, propoxy, butoxy, pentoxy, hexoxy, —CN, benzyl, —NMe₂, -NH₂, —SO₂Me, —COCH₃,

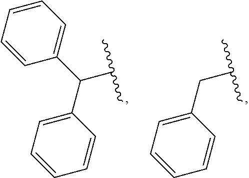 [SMILES: *C(c1ccccc1)c1ccccc1 ; *Cc1ccccc1]

oxetanyl, azetidinyl or 1-methylazetidinyl; wherein each of said methyl, ethyl, propyl(n-propyl or isopropyl), butyl(n-butyl, sec-butyl, iso-butyl or tert-butyl), cyclopropyl, cyclobutyl, cyclopentyl, cyclohexyl, oxetanyl, or azetidinyl is optionally substituted with F, —Cl, methyl, ethyl, propyl(n-propyl or isopropyl), butyl(n-butyl, sec-butyl, iso-butyl or tert-butyl), pentyl, hexyl, heptyl, —OR^{Xad}, —SO₂R^{Xad}, —C(O)R^{Xad};
R^{Xad} is selected from hydrogen, methyl, and ethyl;
even more preferably, R^{Xa} is each independently selected from hydrogen, methyl, ethyl, propyl(n-propyl or isopropyl), butyl(n-butyl, sec-butyl, iso-butyl or tert-butyl), cyclopropyl, cyclobutyl, cyclopentyl, —C₂H₄OH, —C₂H₄OMe, —C₃HoOH, —C₃HOMe, —CH₂OMe, —C(CH₃)₂OH,

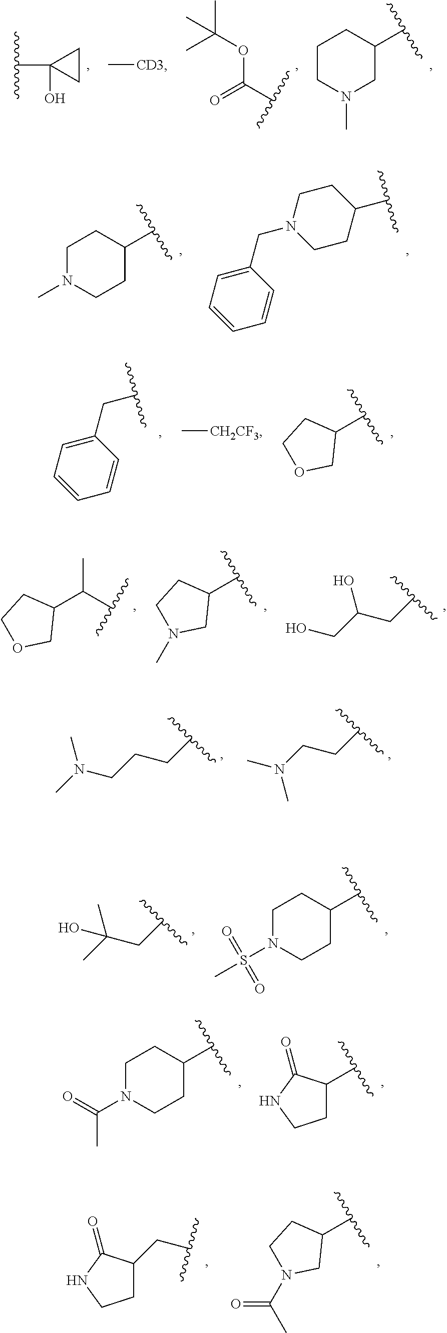

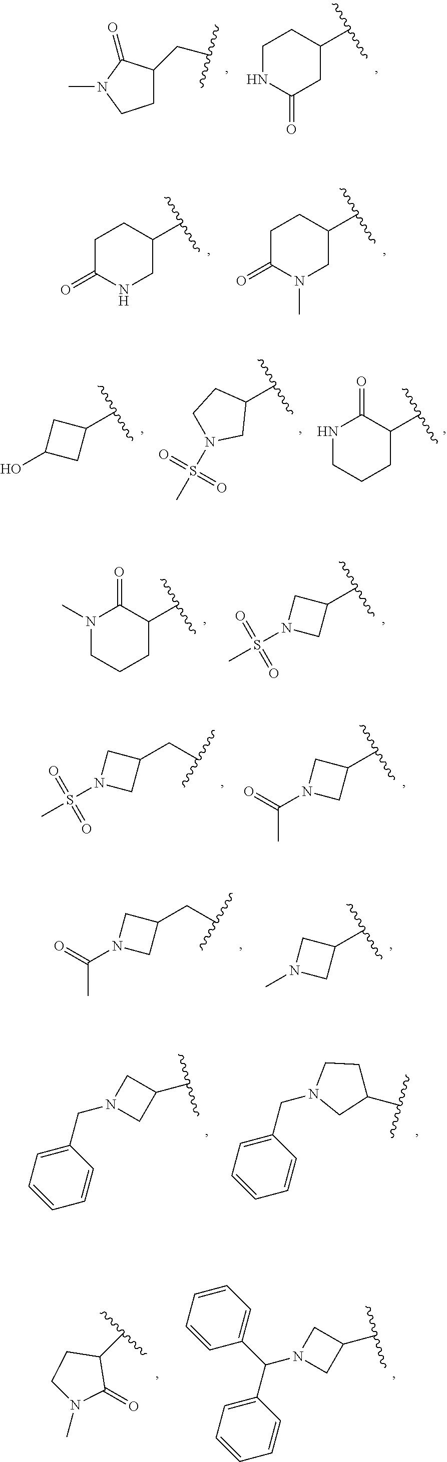

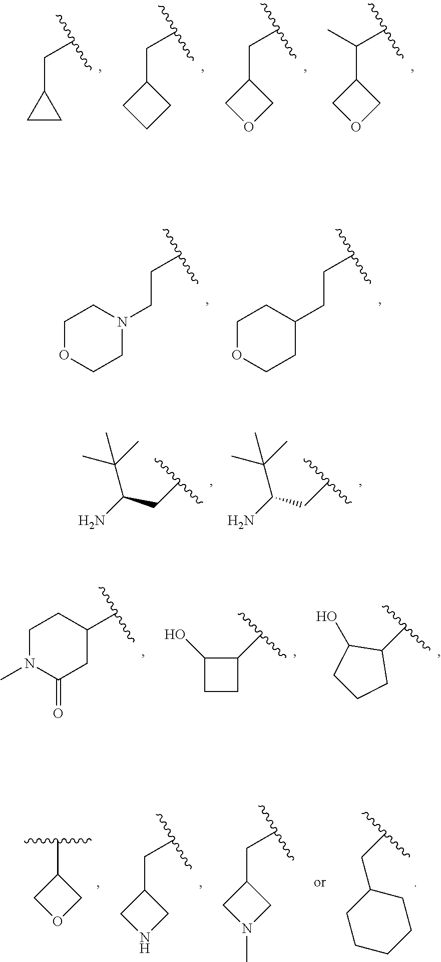

Aspect 16. The compound of anyone of the preceding aspects, wherein R^{Xb} is each independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xba}R^{Xbb}, —OR^{Xba}, —SR^{Xba}, —SO₂ R^{Xba}, —SO₂NR^{Xba}R^{Xbb}, —C(O)R^{Xba}, —CO₂R^{Xba}, —C(O)NR^{Xba}R^{Xbb}, —NR^{Xba}COR^{Xbb}, —NR^{Xba}CO₂R^{Xbb} or —NR^{Xba}SO₂R^{Xbb} or —CN, wherein each of said methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{Xbc};
R^{Xba} and R^{Xbb} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{Xbd};
R^{Xbc} and R^{Xbd} are each independently selected from —F, —Cl, —Br, —I, hydroxy, methyl, ethyl, propyl(n-propyl or isopropyl), butyl(n-butyl, sec-butyl, iso-butyl or tert-butyl), pentyl, hexyl, heptyl, octyl, methoxy, ethoxy, propoxy, butoxy, pentoxy, hexoxy, hepthoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xbe}R^{Xbf}, —OR^{Xbe}, —SR^{Xbe}, —SO₂R^{Xbe}, —SO₂NR^{Xbe}R^{Xbf} C(O)R^{Xbe}, —CO₂R^{Xbe}, —C(O)NR^{Xbe}R^{Xbf}, —NR^{Xbe}COR^{Xbf}, —NR^{Xbe}COO₂R^{Xbf} or —NR^{Xbe}SO₂R^{Xbf} or —CN;
R^{Xbe} and R^{Xbf} are each independently selected from methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, R^{Xb} is independently selected from hydrogen, —F, —Cl, —Br, —I, methyl, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, oxo (═O), —NR^{Xba} R^{Xbb}, —OR^{Xba}, —SR^{Xba}, —SO₂R^{Xba}, —SO₂NR^{Xba}R^{Xbb}, —C(O)R^{Xba}, —CO₂R^{Xba}, —C(O)NR^{Xba}R^{Xbb}, —NR^{Xba}COR^{Xbb}, —NR^{Xba}CO₂R^{Xbb} or —NR^{Xba}SO₂R^{Xbb} or —CN;
R^{Xba} and R^{Xbb} are each independently selected from hydrogen, methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R^{Xb} is independently selected from hydrogen, —F, —Cl, —Br, —I, methyl, -CD₃, ethyl, propyl(n-propyl or isopropyl), butyl(n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NH₂, —OH, methoxy, ethoxy, propoxy, butoxy, pentoxy, hexoxy, hepthoxy, octoxy or —CN;
even more preferably, R^{Xb} is independently selected from hydrogen, —F, —Cl, methyl, -CD₃, ethyl, propyl(n-propyl or isopropyl), butyl(n-butyl, sec-butyl, iso-butyl or tert-butyl), cyclopropyl, cyclobutyl, cyclopentyl, cyclohexyl, —CH₂F, —CHF₂, —CF₃, —CH₂CH₂F, —CH₂CHF₂, —CH₂CF₃, oxo (═O), or —OH.

Aspect 17. The compound of anyone of the preceding aspects, wherein two R^{Xb} together with the atom(s) to which they are attached, form a 3-, 4-, 5-, 6-, 7- or 8-membered ring, said ring comprising 0, 1,2 or 3 heteroatoms independently selected from nitrogen, oxygen or optionally oxidized sulfur as ring member(s); said ring is optionally substituted with at least one substituent R^{Xbc};
R^{Xbc} is each independently selected from —F, —Cl, —Br, —I, hydroxy, methyl, ethyl, propyl(n-propyl or isopropyl), butyl(n-butyl, sec-butyl, iso-butyl or tert-butyl), pentyl, hexyl, heptyl, octyl, methoxy, ethoxy, propoxy, butoxy, pentoxy, hexoxy, hepthoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xbe}R^{Xbf}, —OR^{Xbe}, —SR^{Xbe}, —SO₂R^{Xbe}, —SO₂NR^{Xbe}R^{Xbf}, —C(O)R^{Xbe}, —CO₂R^{Xbe}, —C(O)NR^{Xbe}R^{Xbf}, —NR^{Xbe}COR^{Xbf}, —NR^{Xbe}COO₂R^{Xbf} or —NR^{Xbe}SO₂R^{Xbf} or —CN;
R^{Xbe} and R^{Xbf} are each independently selected from methyl, ethyl, propyl(n-propyl or isopropyl), butyl(n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, two R^{Xb} together with the atom(s) to which they are attached, form a 3-, 4-, 5- or 6-membered ring; said ring is optionally substituted with at least one substituent R^{Xbc}.
R^{Xbc} is each independently selected from —F, —Cl, —Br, —I, hydroxy, methyl, ethyl, propyl(n-propyl or isopropyl), butyl(n-butyl, sec-butyl, iso-butyl or tert-butyl), methoxy, ethoxy, propoxy, butoxy, cyclopropyl, cyclobutyl, cyclopentyl, cyclohexyl, oxo (═O) or —CN;
more preferably, two R^{Xb} together with the atom(s) to which they are attached, form a 3-, 4-, 5- or 6-membered ring.

Aspect 18. The compound of anyone of the preceding aspects, wherein the

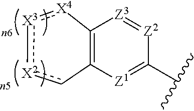

moiety is selected from

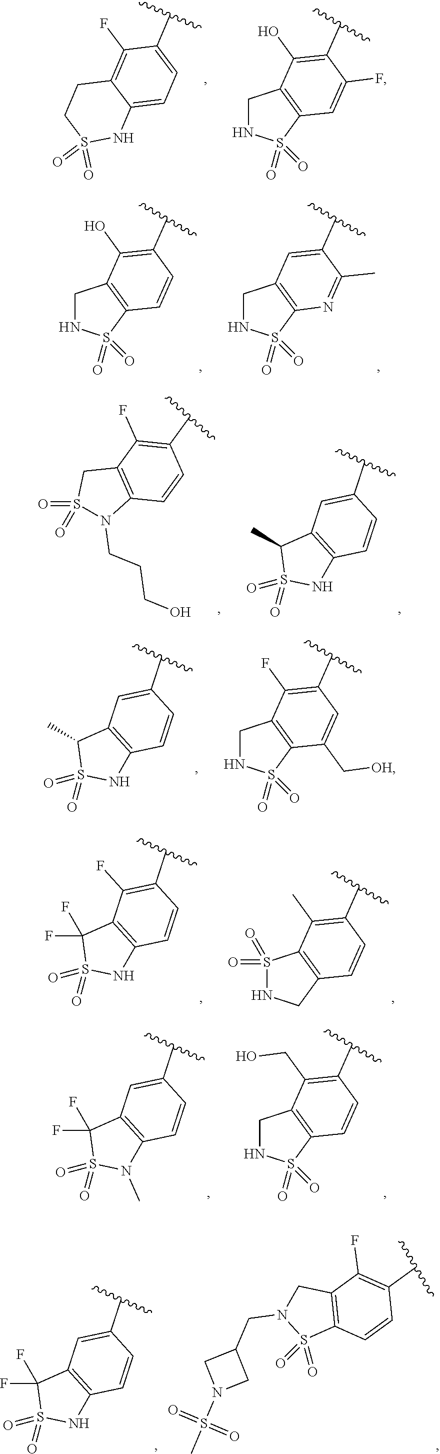

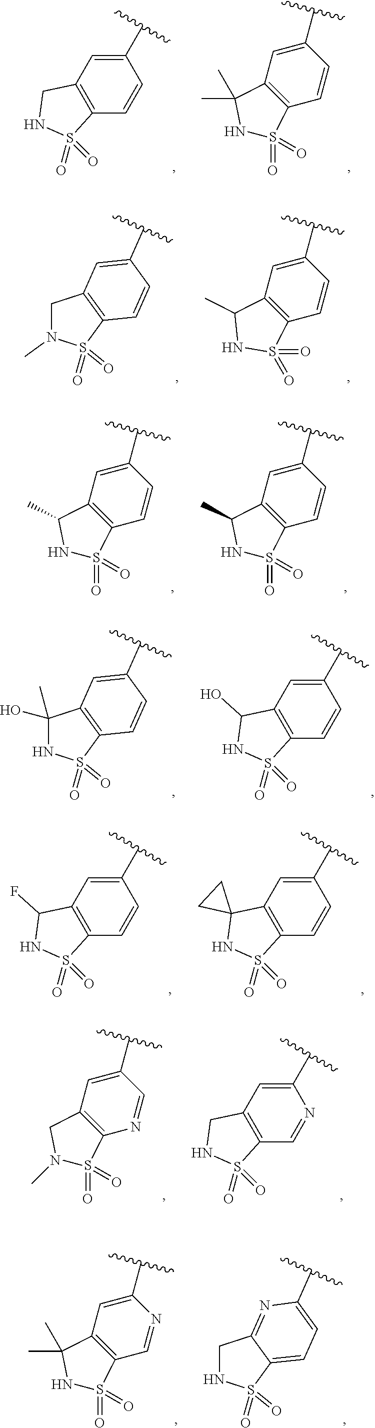

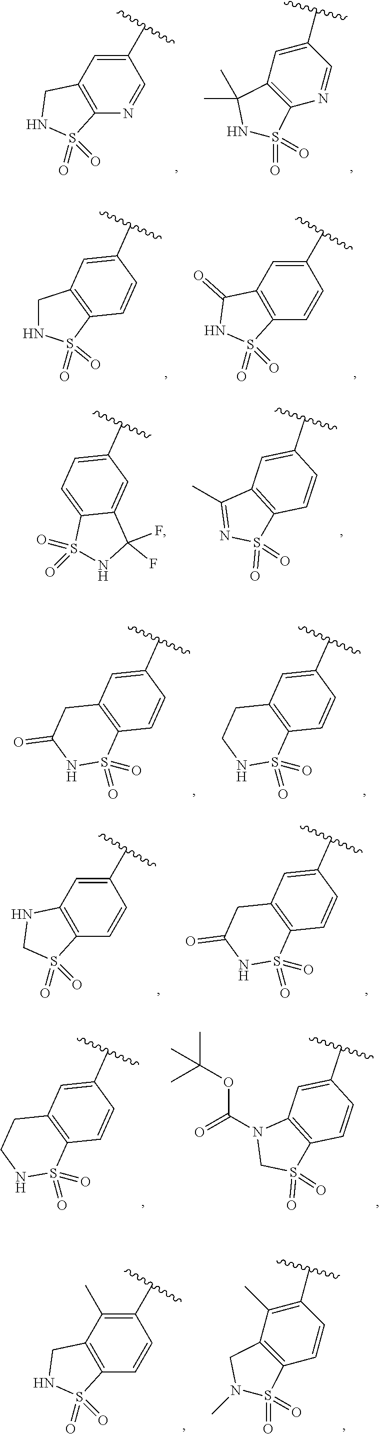

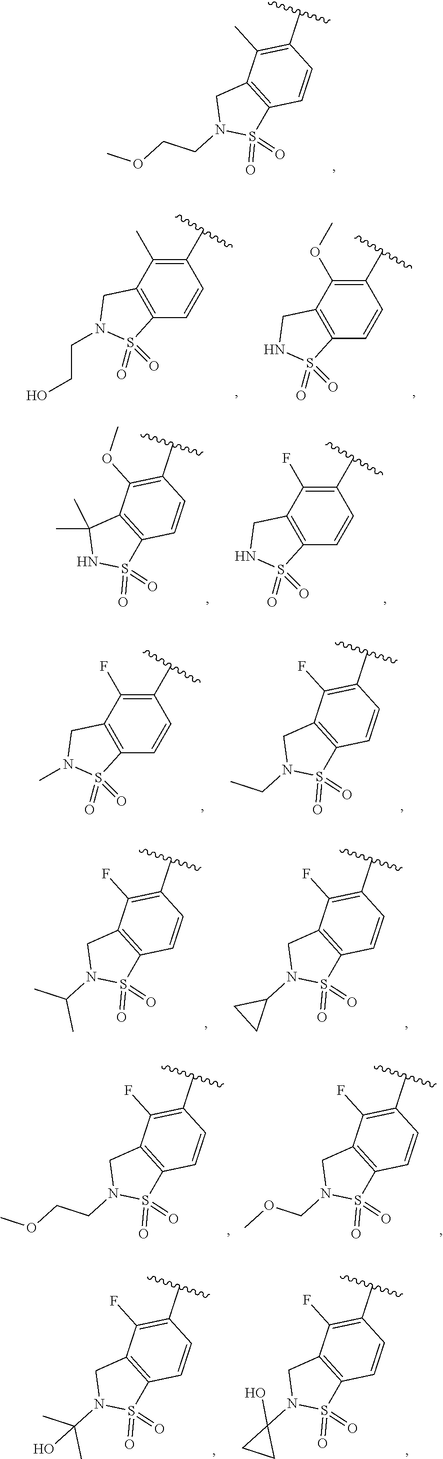

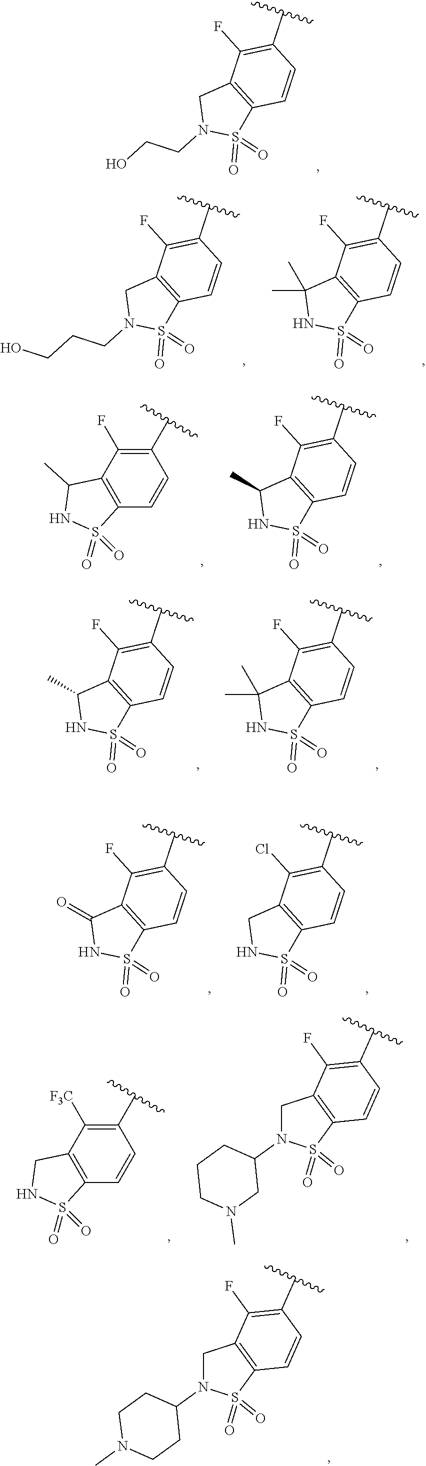

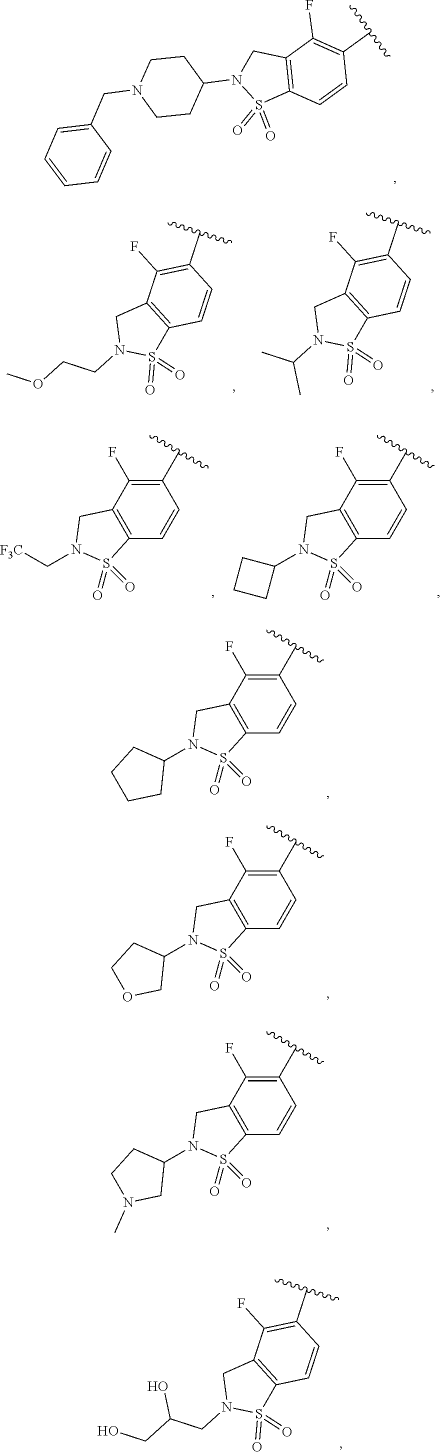

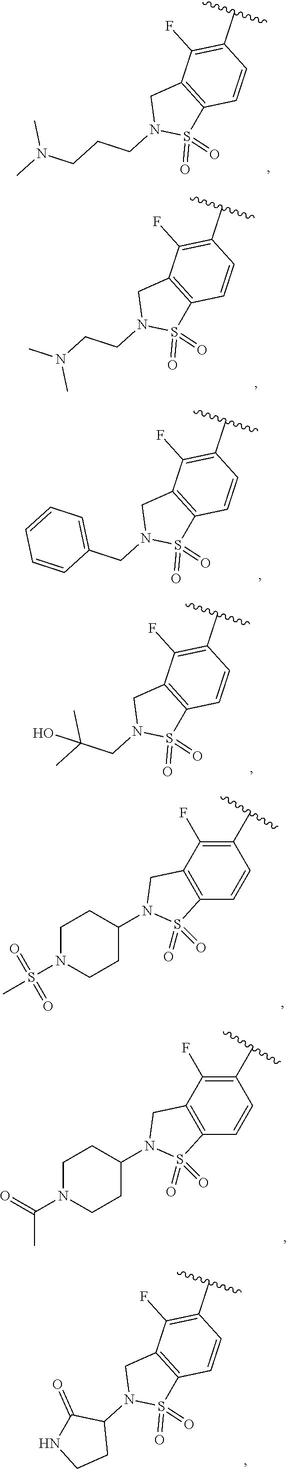

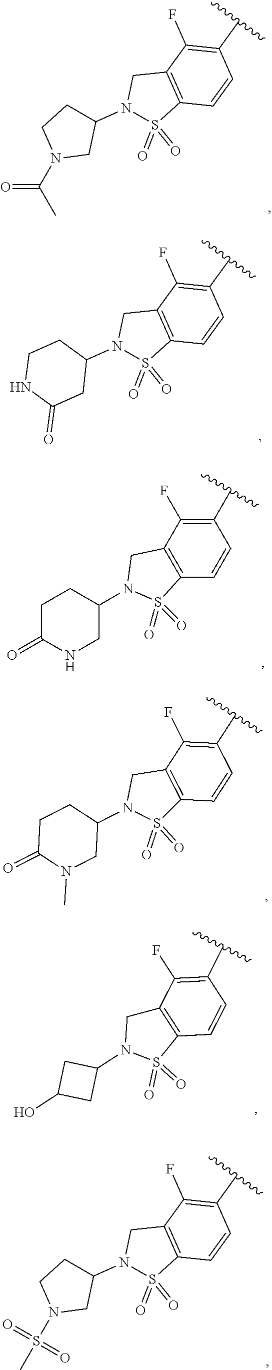 [SMILES: *c1ccc2c(c1F)CN(C1CCN(C(C)=O)C1)S2(=O)=O ; *c1ccc2c(c1F)CN(C1CCNC(=O)C1)S2(=O)=O ; *c1ccc2c(c1F)CN(C1CCC(=O)NC1)S2(=O)=O ; *c1ccc2c(c1F)CN(C1CCC(=O)N(C)C1)S2(=O)=O ; *c1ccc2c(c1F)CN(C1CC(O)C1)S2(=O)=O ; *c1ccc2c(c1F)CN(C1CCN(S(C)(=O)=O)C1)S2(=O)=O]

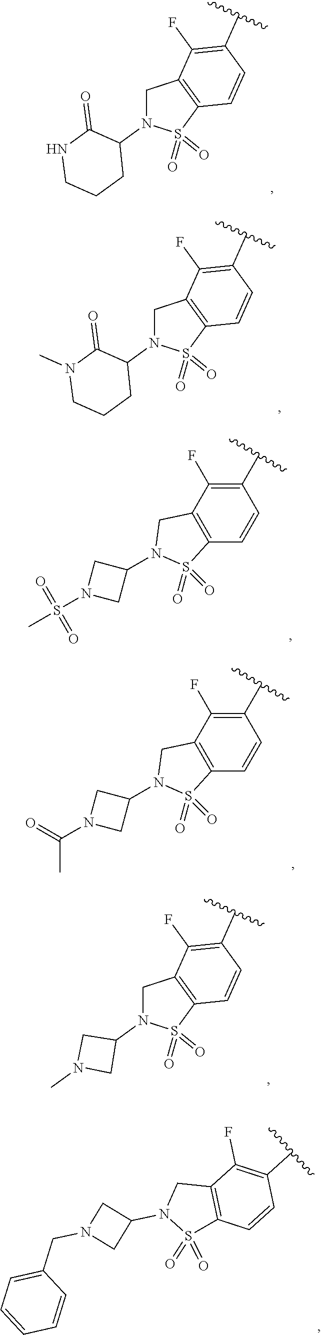 [SMILES: *c1ccc2c(c1F)CN(C1CCCNC1=O)S2(=O)=O ; *c1ccc2c(c1F)CN(C1CCCN(C)C1=O)S2(=O)=O ; *c1ccc2c(c1F)CN(C1CN(S(C)(=O)=O)C1)S2(=O)=O ; *c1ccc2c(c1F)CN(C1CN(C(C)=O)C1)S2(=O)=O ; *c1ccc2c(c1F)CN(C1CN(C)C1)S2(=O)=O ; *c1ccc2c(c1F)CN(C1CN(Cc3ccccc3)C1)S2(=O)=O]

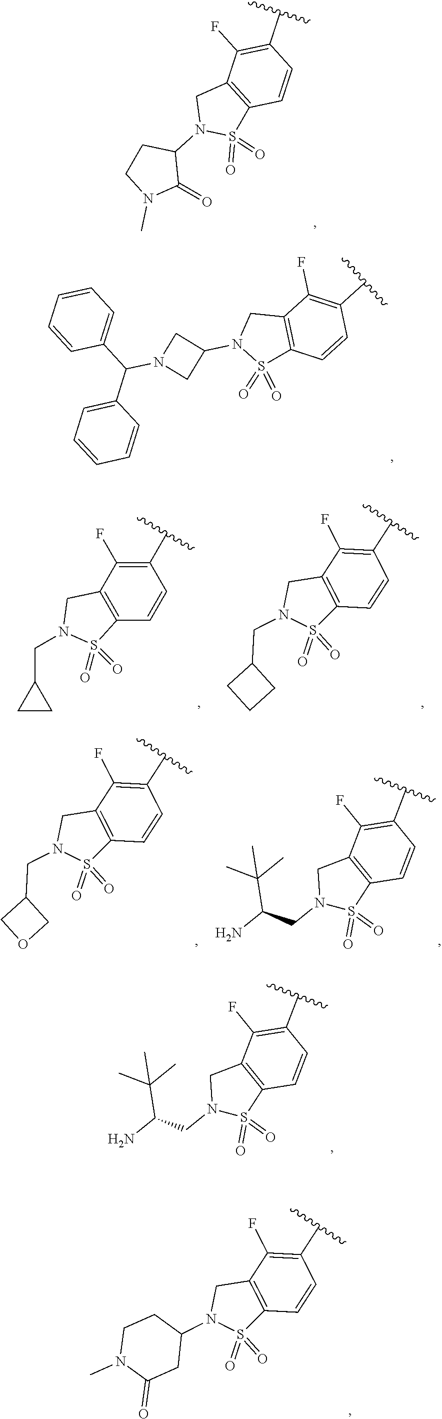

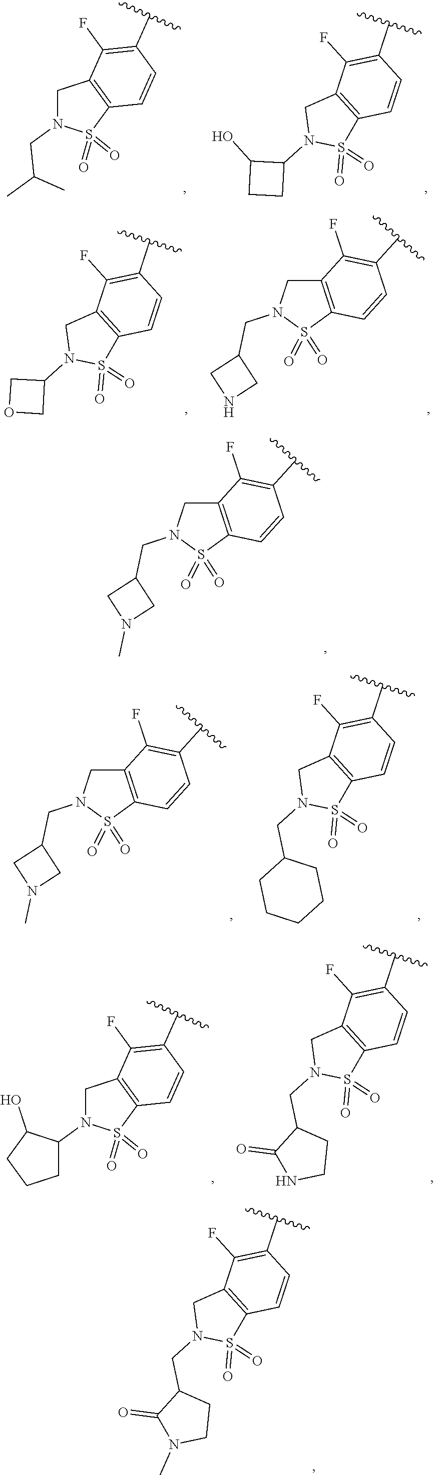

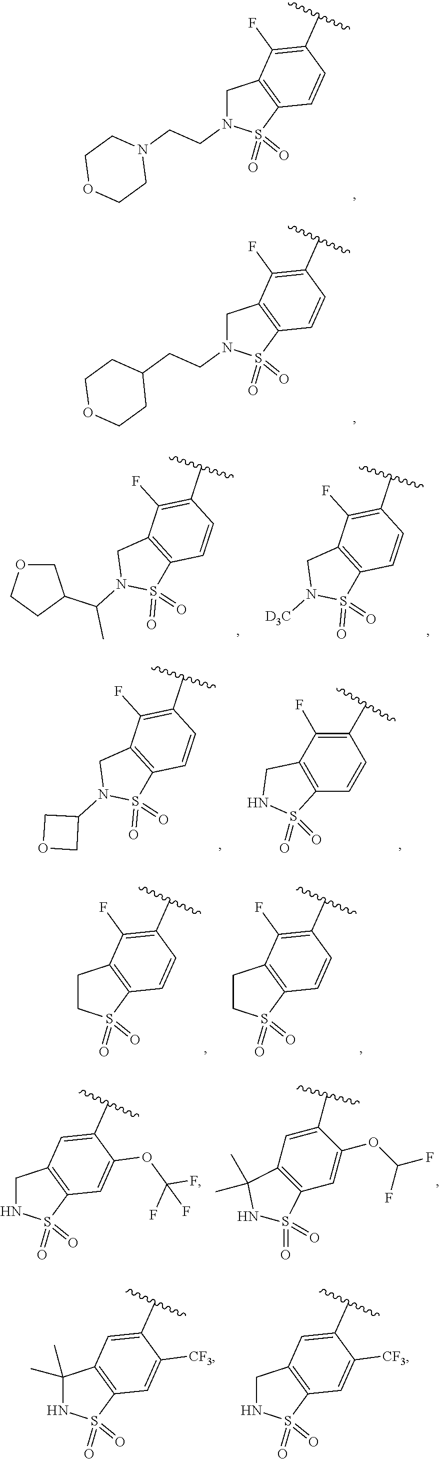

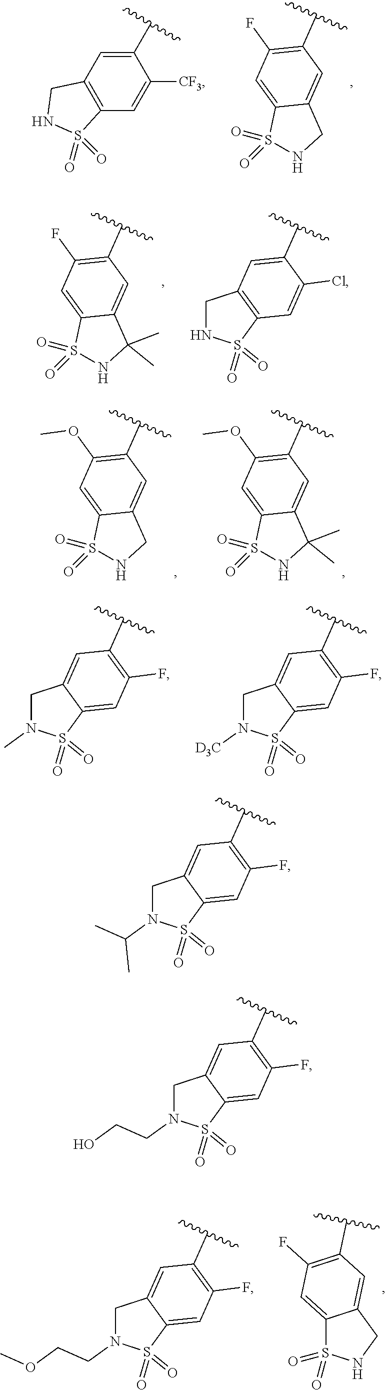

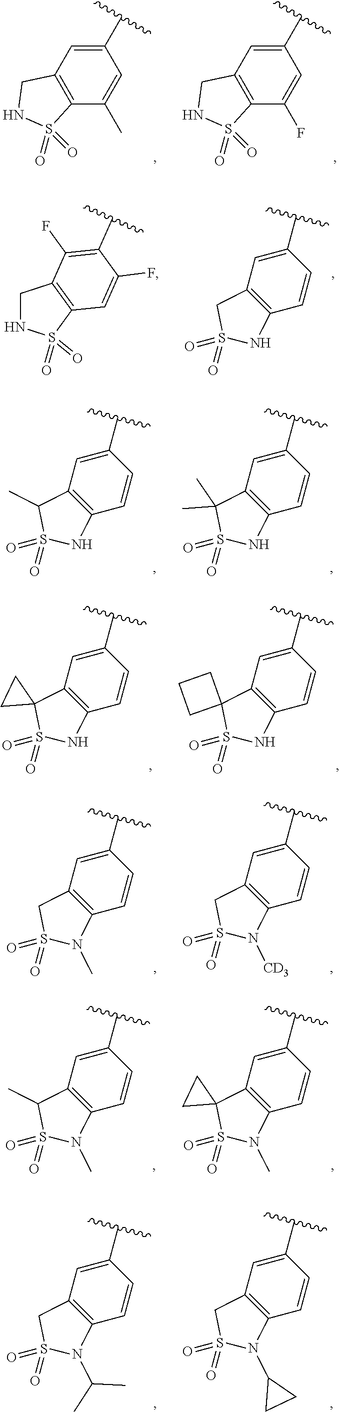

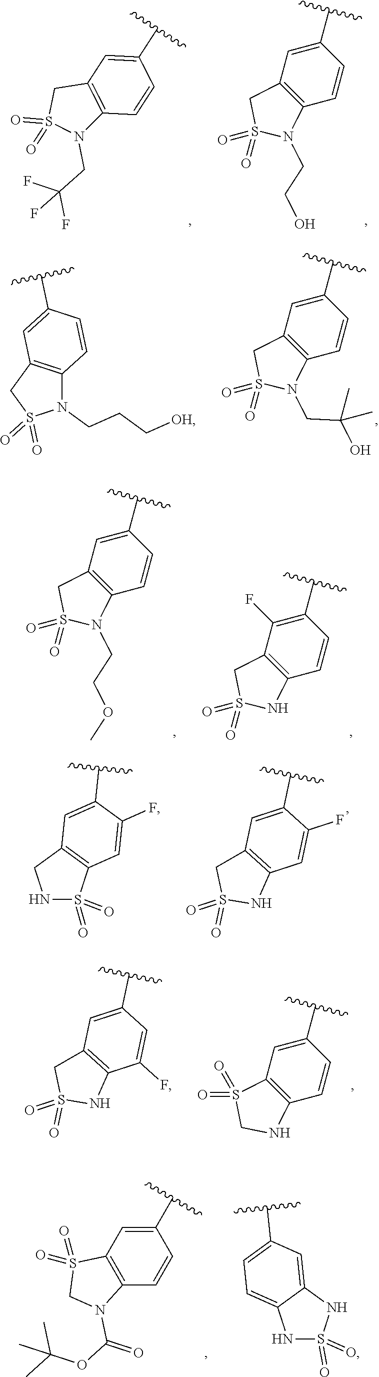

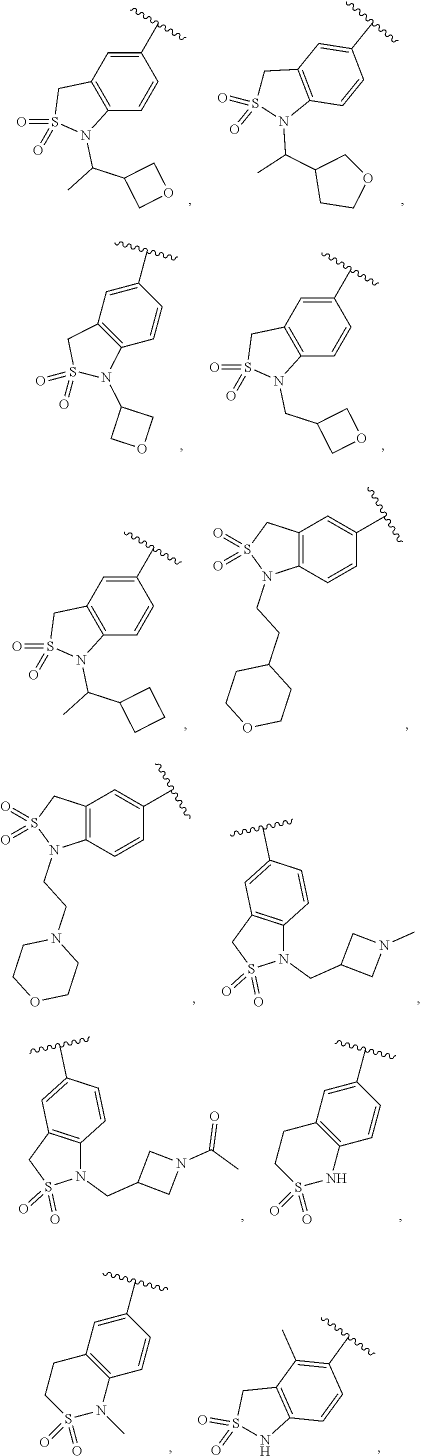

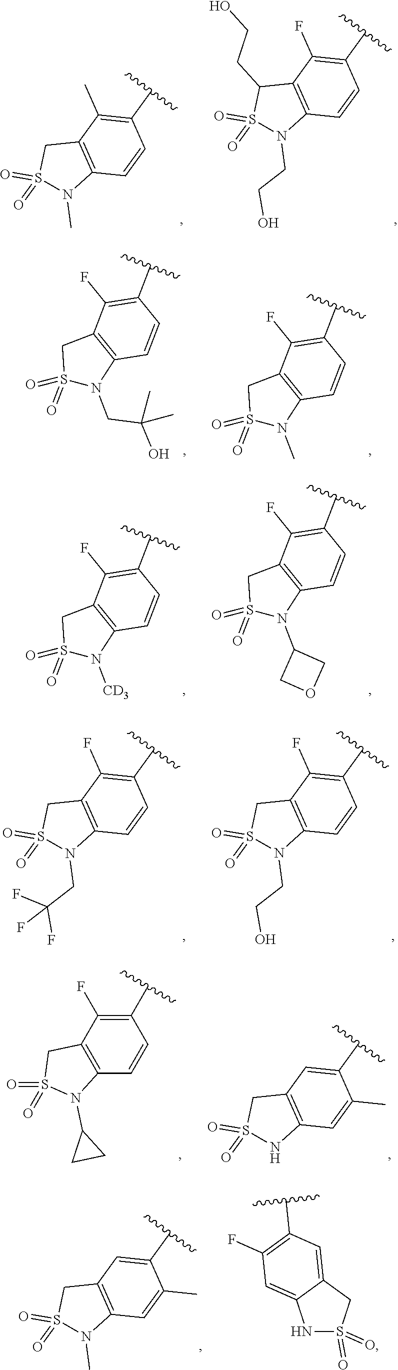

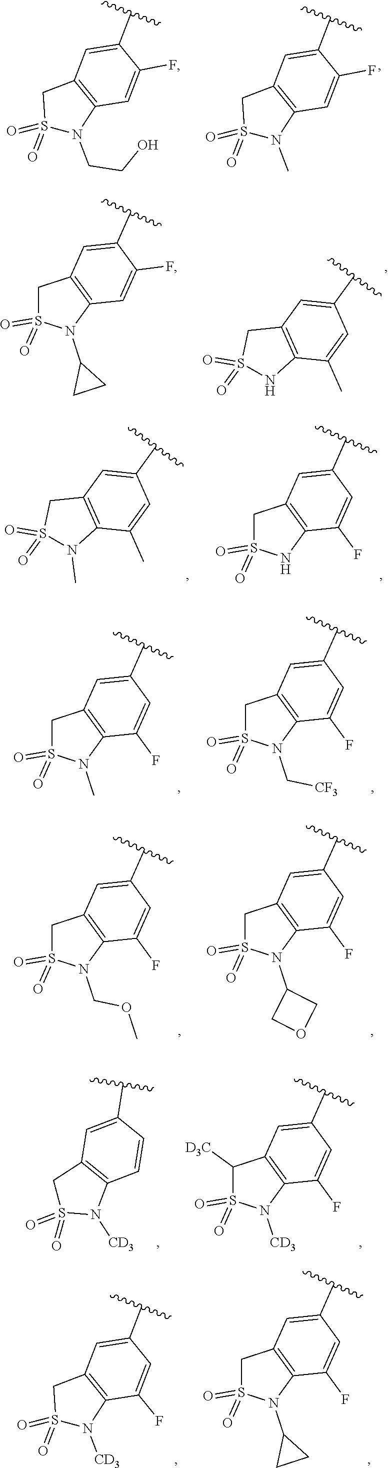

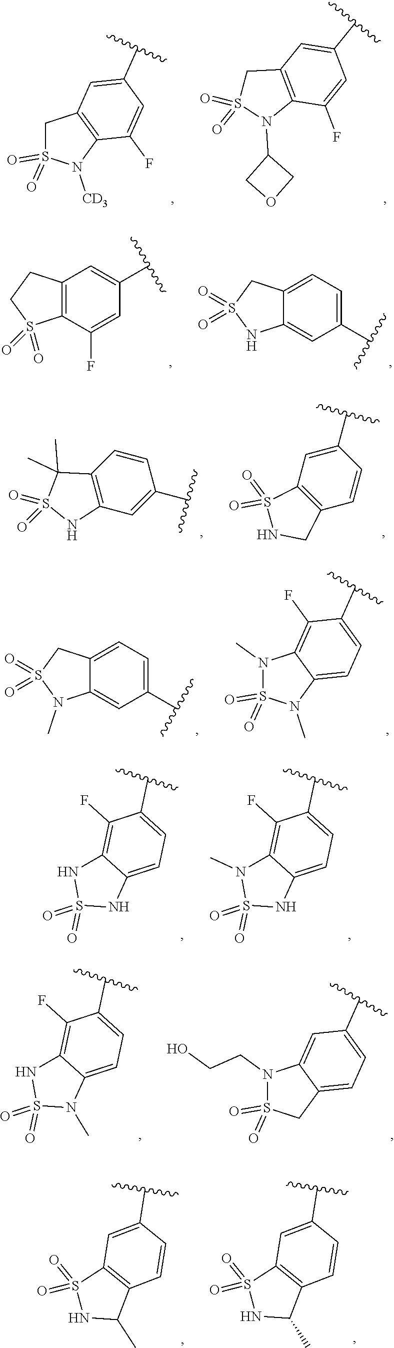

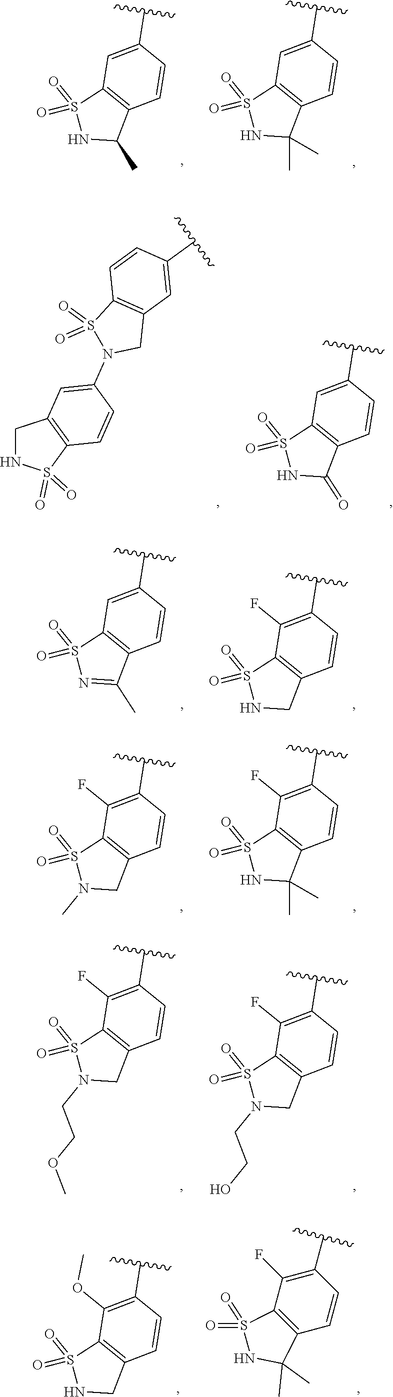

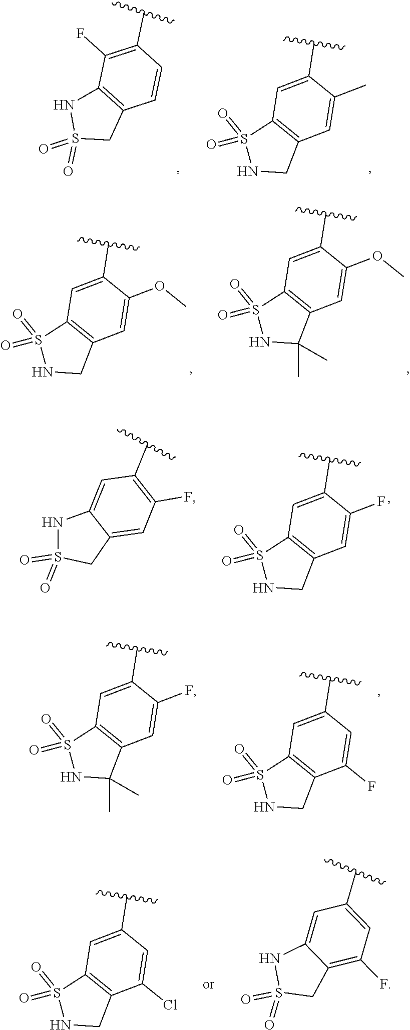

Aspect 19. The compound of anyone of the preceding aspects, wherein the compound is selected from

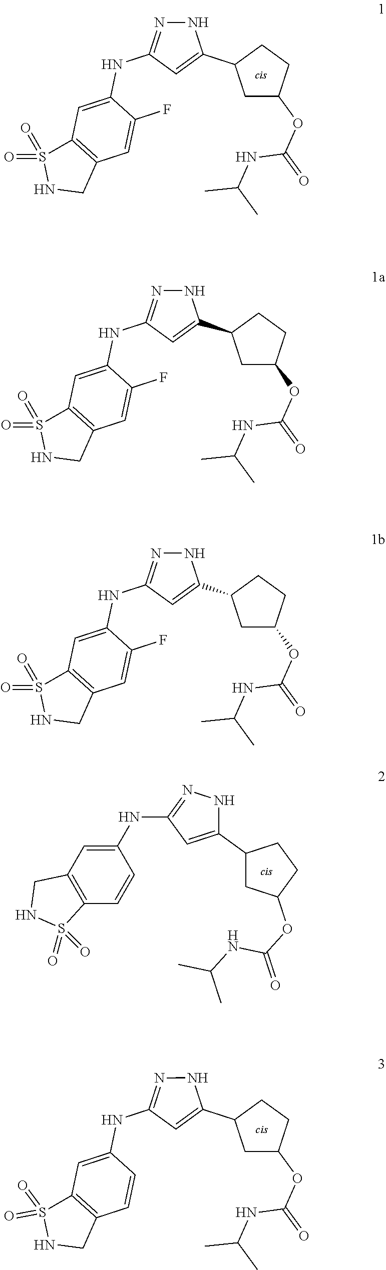 [SMILES: CC(C)NC(=O)OC1CCC(c2cc(Nc3cc4c(cc3F)CNS4(=O)=O)n[nH]2)C1 ; CC(C)NC(=O)O[C@@H]1CC[C@H](c2cc(Nc3cc4c(cc3F)CNS4(=O)=O)n[nH]2)C1 ; CC(C)NC(=O)O[C@H]1CC[C@@H](c2cc(Nc3cc4c(cc3F)CNS4(=O)=O)n[nH]2)C1 ; CC(C)NC(=O)OC1CCC(c2cc(Nc3ccc4c(c3)CNS4(=O)=O)n[nH]2)C1 ; CC(C)NC(=O)OC1CCC(c2cc(Nc3ccc4c(c3)S(=O)(=O)NC4)n[nH]2)C1]

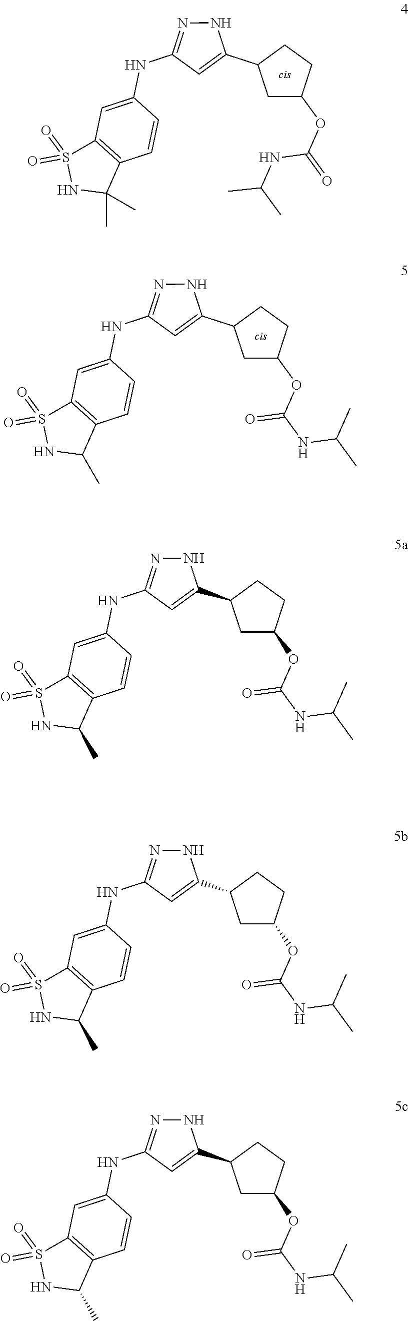 [SMILES: CC(C)NC(=O)OC1CCC(c2cc(Nc3ccc4c(c3)S(=O)(=O)NC4(C)C)n[nH]2)C1 ; CC(C)NC(=O)OC1CCC(c2cc(Nc3ccc4c(c3)S(=O)(=O)NC4C)n[nH]2)C1 ; CC(C)NC(=O)O[C@@H]1CC[C@H](c2cc(Nc3ccc4c(c3)S(=O)(=O)N[C@@H]4C)n[nH]2)C1 ; CC(C)NC(=O)O[C@H]1CC[C@@H](c2cc(Nc3ccc4c(c3)S(=O)(=O)N[C@@H]4C)n[nH]2)C1 ; CC(C)NC(=O)O[C@@H]1CC[C@H](c2cc(Nc3ccc4c(c3)S(=O)(=O)N[C@H]4C)n[nH]2)C1]

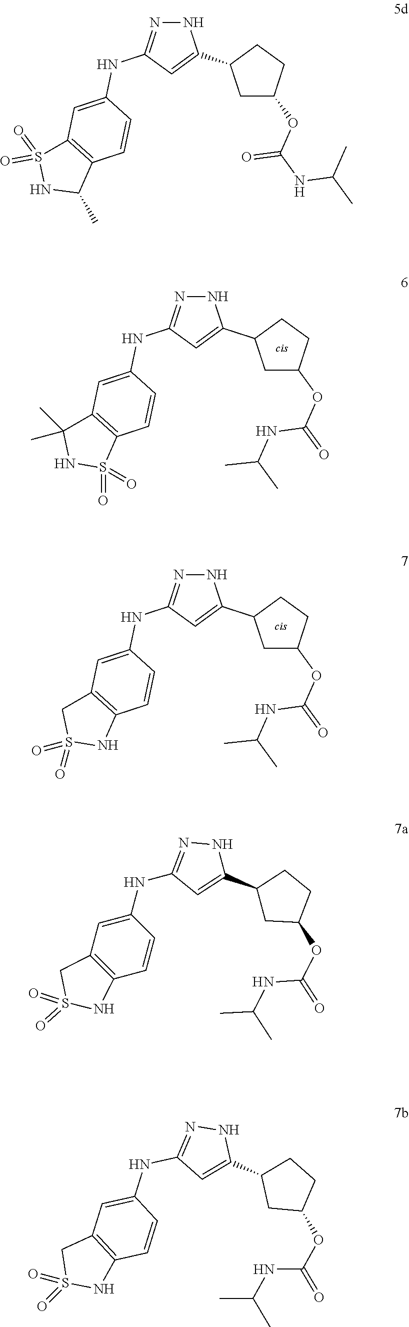

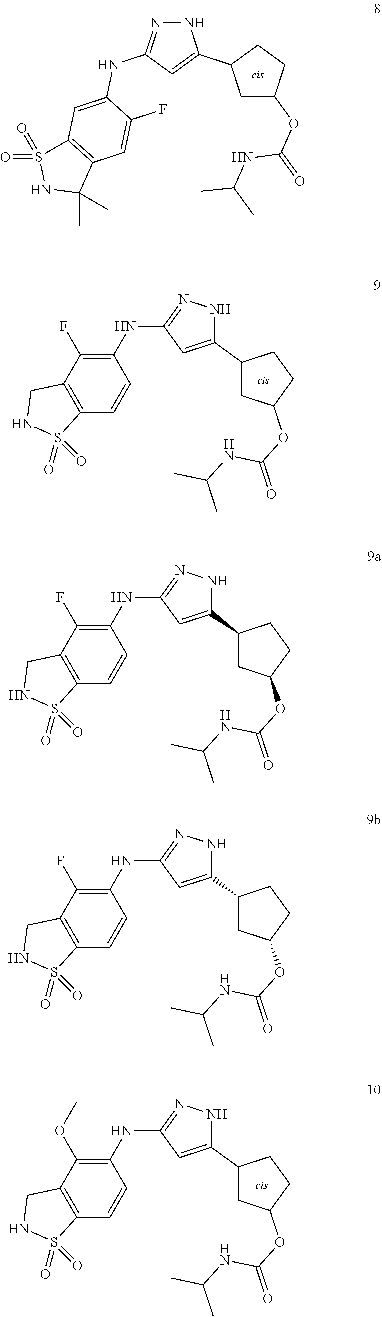 [SMILES: CC(C)NC(=O)OC1CCC(c2cc(Nc3cc4c(cc3F)C(C)(C)NS4(=O)=O)n[nH]2)C1 ; CC(C)NC(=O)OC1CCC(c2cc(Nc3ccc4c(c3F)CNS4(=O)=O)n[nH]2)C1 ; CC(C)NC(=O)O[C@@H]1CC[C@H](c2cc(Nc3ccc4c(c3F)CNS4(=O)=O)n[nH]2)C1 ; CC(C)NC(=O)O[C@H]1CC[C@@H](c2cc(Nc3ccc4c(c3F)CNS4(=O)=O)n[nH]2)C1 ; COc1c(Nc2cc(C3CCC(OC(=O)NC(C)C)C3)[nH]n2)ccc2c1CNS2(=O)=O]

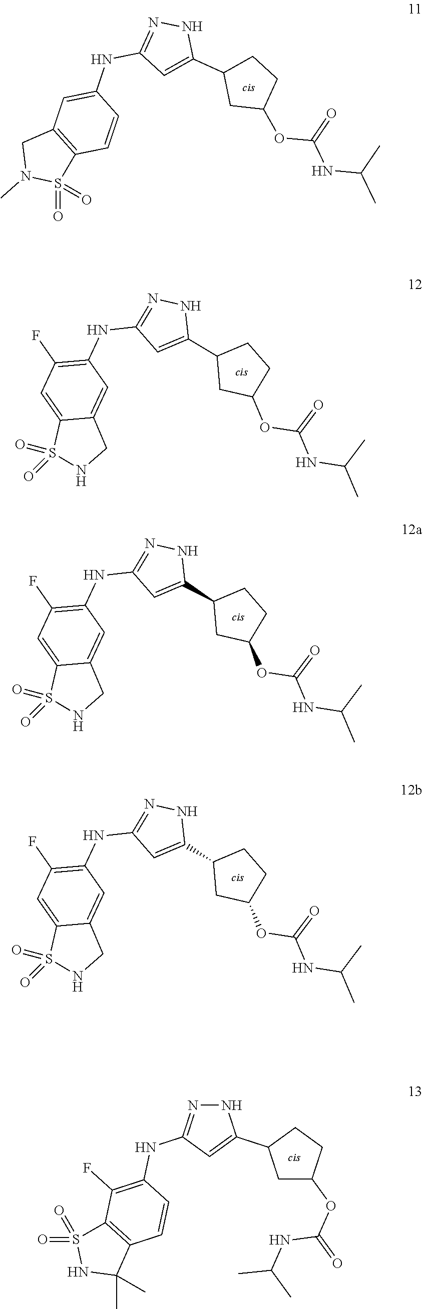 [SMILES: CC(C)NC(=O)OC1CCC(c2cc(Nc3ccc4c(c3)CN(C)S4(=O)=O)n[nH]2)C1 ; CC(C)NC(=O)OC1CCC(c2cc(Nc3cc4c(cc3F)S(=O)(=O)NC4)n[nH]2)C1 ; CC(C)NC(=O)O[C@@H]1CC[C@H](c2cc(Nc3cc4c(cc3F)S(=O)(=O)NC4)n[nH]2)C1 ; CC(C)NC(=O)O[C@H]1CC[C@@H](c2cc(Nc3cc4c(cc3F)S(=O)(=O)NC4)n[nH]2)C1 ; CC(C)NC(=O)OC1CCC(c2cc(Nc3ccc4c(c3F)S(=O)(=O)NC4(C)C)n[nH]2)C1]

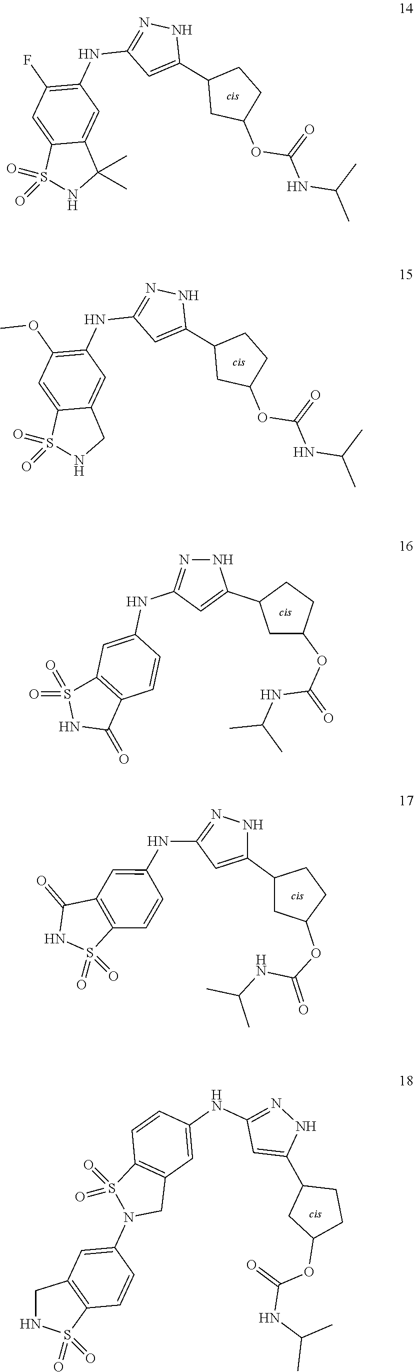

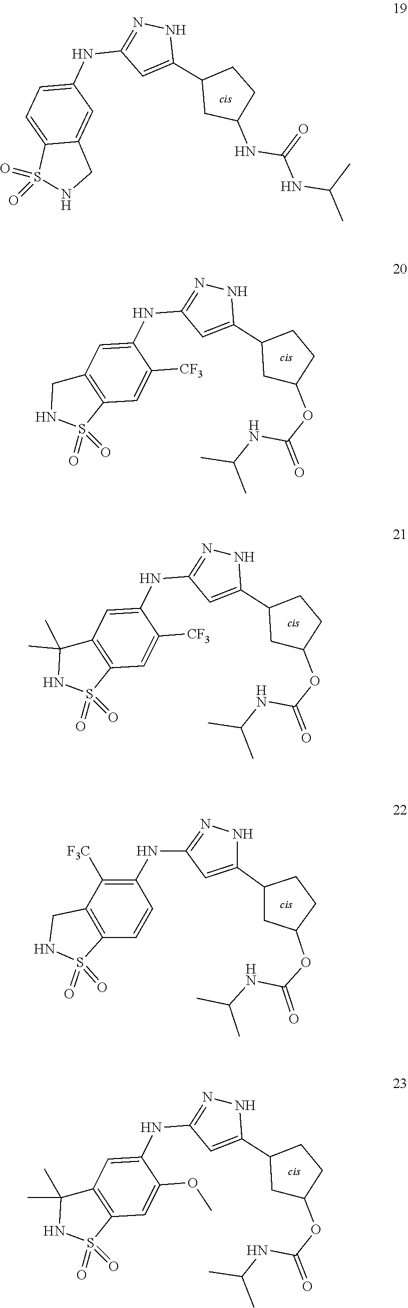

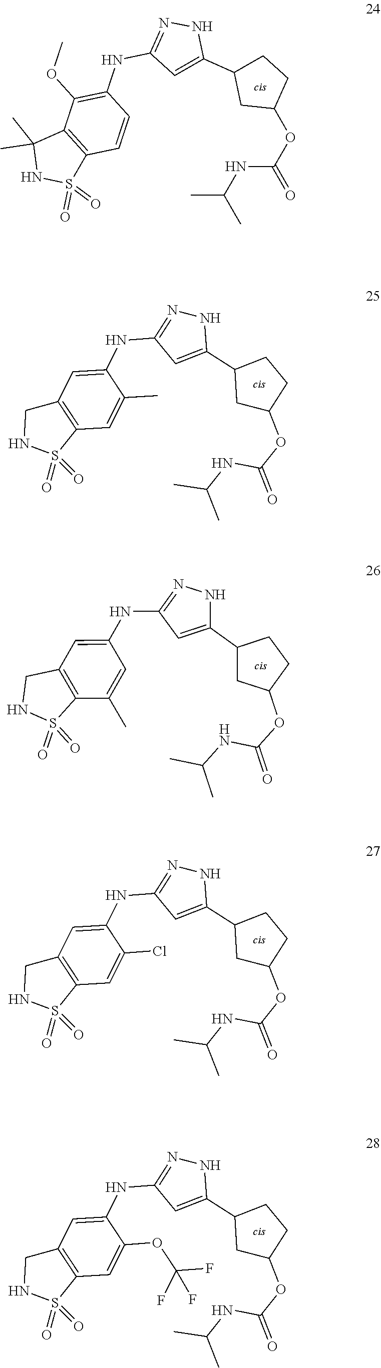 [SMILES: COc1c(Nc2cc(C3CCC(OC(=O)NC(C)C)C3)[nH]n2)ccc2c1C(C)(C)NS2(=O)=O ; Cc1cc2c(cc1Nc1cc(C3CCC(OC(=O)NC(C)C)C3)[nH]n1)CNS2(=O)=O ; Cc1cc(Nc2cc(C3CCC(OC(=O)NC(C)C)C3)[nH]n2)cc2c1S(=O)(=O)NC2 ; CC(C)NC(=O)OC1CCC(c2cc(Nc3cc4c(cc3Cl)S(=O)(=O)NC4)n[nH]2)C1 ; CC(C)NC(=O)OC1CCC(c2cc(Nc3cc4c(cc3OC(F)(F)F)S(=O)(=O)NC4)n[nH]2)C1]

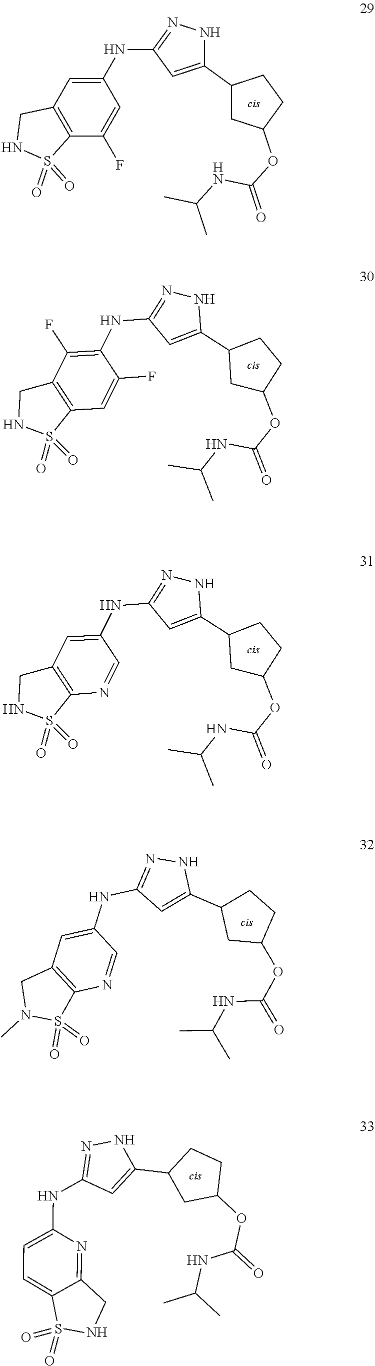 [SMILES: CC(C)NC(=O)OC1CCC(c2cc(Nc3cc(F)c4c(c3)CNS4(=O)=O)n[nH]2)C1 ; CC(C)NC(=O)OC1CCC(c2cc(Nc3c(F)cc4c(c3F)CNS4(=O)=O)n[nH]2)C1 ; CC(C)NC(=O)OC1CCC(c2cc(Nc3cnc4c(c3)CNS4(=O)=O)n[nH]2)C1 ; CC(C)NC(=O)OC1CCC(c2cc(Nc3cnc4c(c3)CN(C)S4(=O)=O)n[nH]2)C1 ; CC(C)NC(=O)OC1CCC(c2cc(Nc3ccc4c(n3)CNS4(=O)=O)n[nH]2)C1]

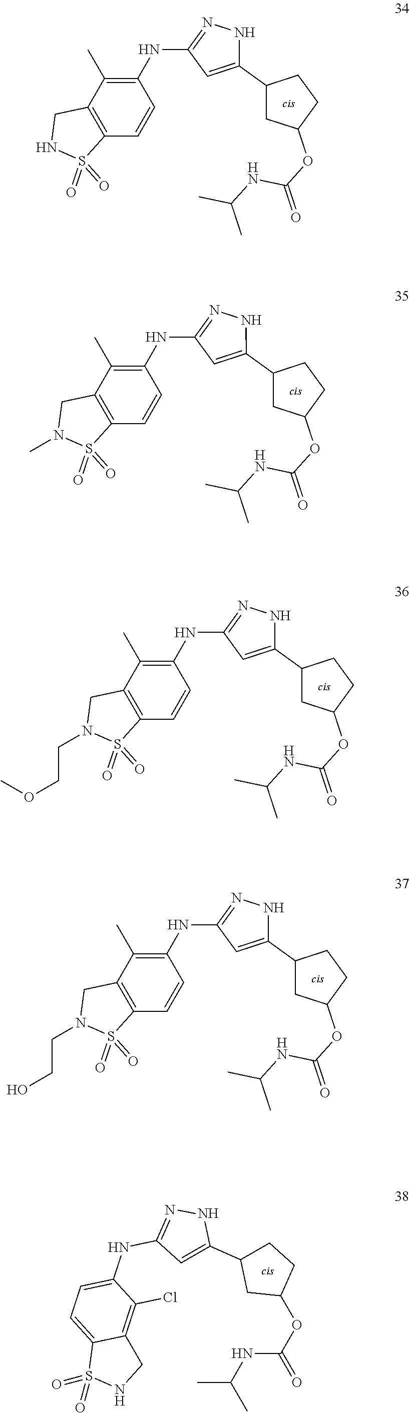 [SMILES: Cc1c(Nc2cc(C3CCC(OC(=O)NC(C)C)C3)[nH]n2)ccc2c1CNS2(=O)=O ; Cc1c(Nc2cc(C3CCC(OC(=O)NC(C)C)C3)[nH]n2)ccc2c1CN(C)S2(=O)=O ; COCCN1Cc2c(ccc(Nc3cc(C4CCC(OC(=O)NC(C)C)C4)[nH]n3)c2C)S1(=O)=O ; Cc1c(Nc2cc(C3CCC(OC(=O)NC(C)C)C3)[nH]n2)ccc2c1CN(CCO)S2(=O)=O ; CC(C)NC(=O)OC1CCC(c2cc(Nc3ccc4c(c3Cl)CNS4(=O)=O)n[nH]2)C1]

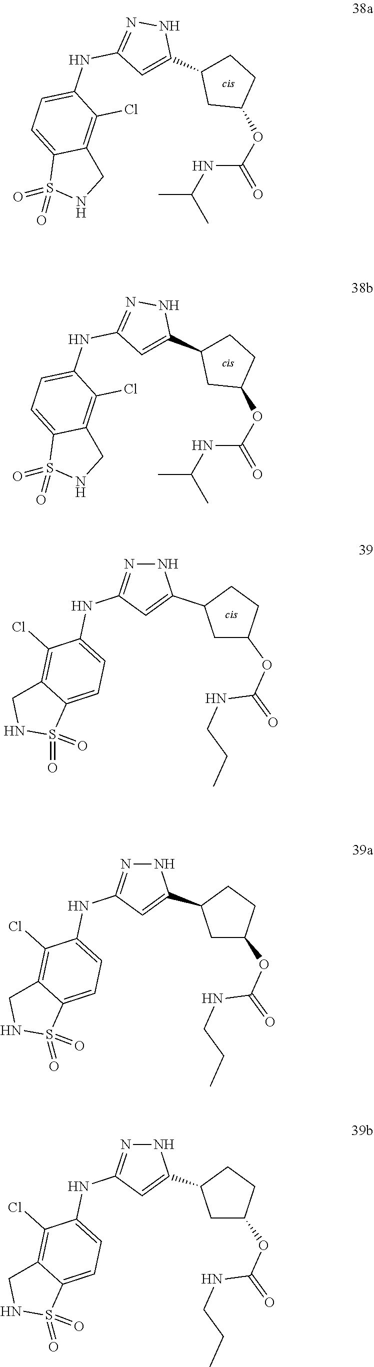 [SMILES: CC(C)NC(=O)O[C@H]1CC[C@@H](c2cc(Nc3ccc4c(c3Cl)CNS4(=O)=O)n[nH]2)C1 ; CC(C)NC(=O)O[C@@H]1CC[C@H](c2cc(Nc3ccc4c(c3Cl)CNS4(=O)=O)n[nH]2)C1 ; CCCNC(=O)OC1CCC(c2cc(Nc3ccc4c(c3Cl)CNS4(=O)=O)n[nH]2)C1 ; CCCNC(=O)O[C@@H]1CC[C@H](c2cc(Nc3ccc4c(c3Cl)CNS4(=O)=O)n[nH]2)C1 ; CCCNC(=O)O[C@H]1CC[C@@H](c2cc(Nc3ccc4c(c3Cl)CNS4(=O)=O)n[nH]2)C1]

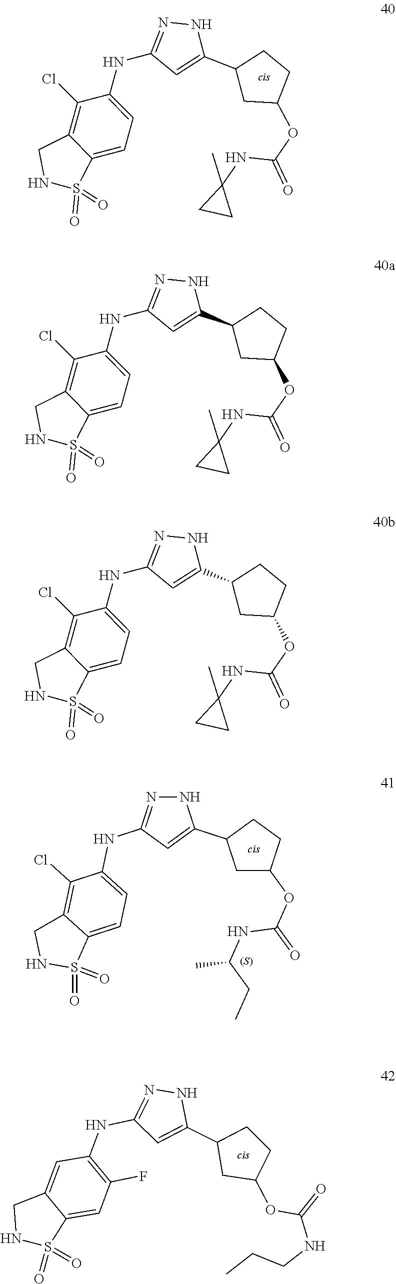

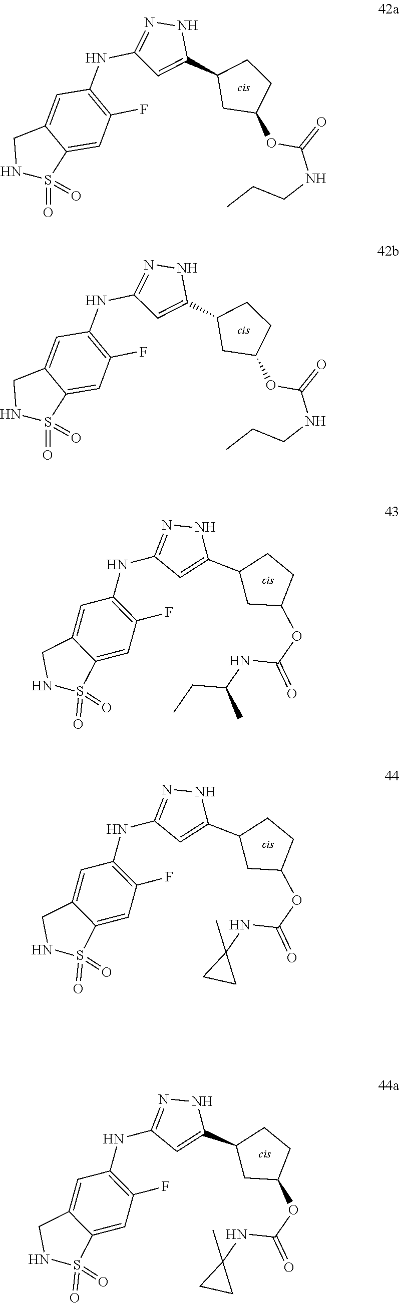 [SMILES: CCCNC(=O)O[C@@H]1CC[C@H](c2cc(Nc3cc4c(cc3F)S(=O)(=O)NC4)n[nH]2)C1 ; CCCNC(=O)O[C@H]1CC[C@@H](c2cc(Nc3cc4c(cc3F)S(=O)(=O)NC4)n[nH]2)C1 ; CC[C@H](C)NC(=O)OC1CCC(c2cc(Nc3cc4c(cc3F)S(=O)(=O)NC4)n[nH]2)C1 ; CC1(NC(=O)OC2CCC(c3cc(Nc4cc5c(cc4F)S(=O)(=O)NC5)n[nH]3)C2)CC1 ; CC1(NC(=O)O[C@@H]2CC[C@H](c3cc(Nc4cc5c(cc4F)S(=O)(=O)NC5)n[nH]3)C2)CC1]

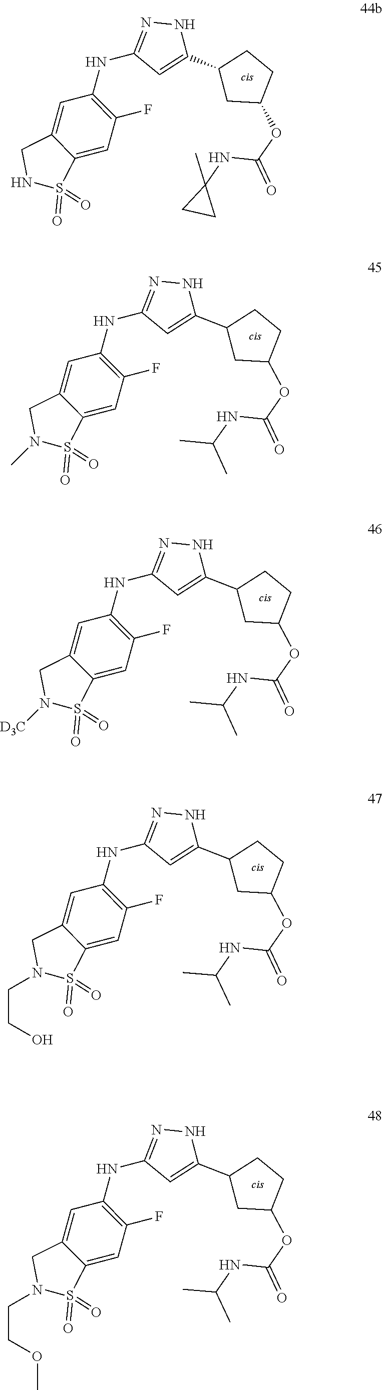 [SMILES: CC1(NC(=O)O[C@H]2CC[C@@H](c3cc(Nc4cc5c(cc4F)S(=O)(=O)NC5)n[nH]3)C2)CC1 ; CC(C)NC(=O)OC1CCC(c2cc(Nc3cc4c(cc3F)S(=O)(=O)N(C)C4)n[nH]2)C1 ; [2H]C([2H])([2H])N1Cc2cc(Nc3cc(C4CCC(OC(=O)NC(C)C)C4)[nH]n3)c(F)cc2S1(=O)=O ; CC(C)NC(=O)OC1CCC(c2cc(Nc3cc4c(cc3F)S(=O)(=O)N(CCO)C4)n[nH]2)C1 ; COCCN1Cc2cc(Nc3cc(C4CCC(OC(=O)NC(C)C)C4)[nH]n3)c(F)cc2S1(=O)=O]

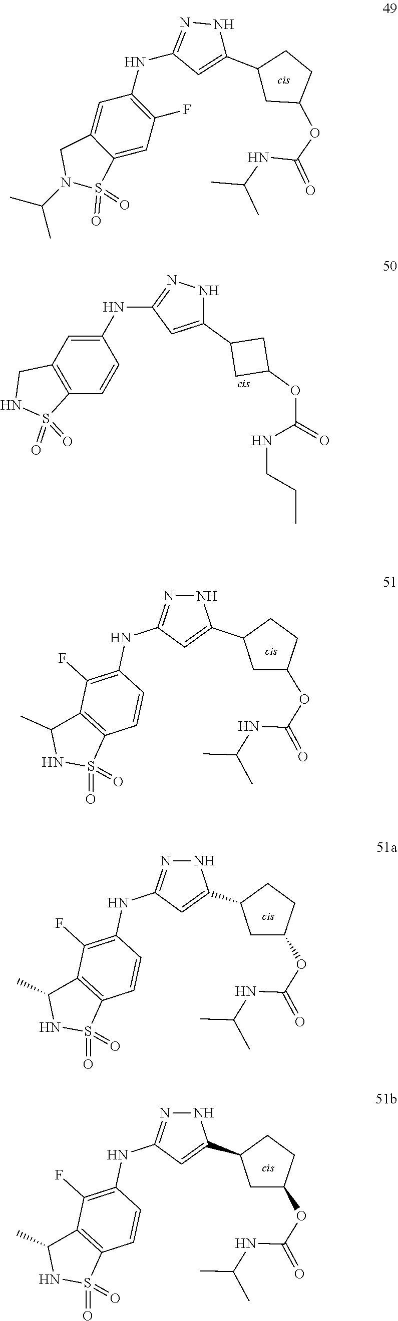 [SMILES: CC(C)NC(=O)OC1CCC(c2cc(Nc3cc4c(cc3F)S(=O)(=O)N(C(C)C)C4)n[nH]2)C1 ; CCCNC(=O)OC1CC(c2cc(Nc3ccc4c(c3)CNS4(=O)=O)n[nH]2)C1 ; CC(C)NC(=O)OC1CCC(c2cc(Nc3ccc4c(c3F)C(C)NS4(=O)=O)n[nH]2)C1 ; CC(C)NC(=O)O[C@H]1CC[C@@H](c2cc(Nc3ccc4c(c3F)[C@@H](C)NS4(=O)=O)n[nH]2)C1 ; CC(C)NC(=O)O[C@@H]1CC[C@H](c2cc(Nc3ccc4c(c3F)[C@@H](C)NS4(=O)=O)n[nH]2)C1]

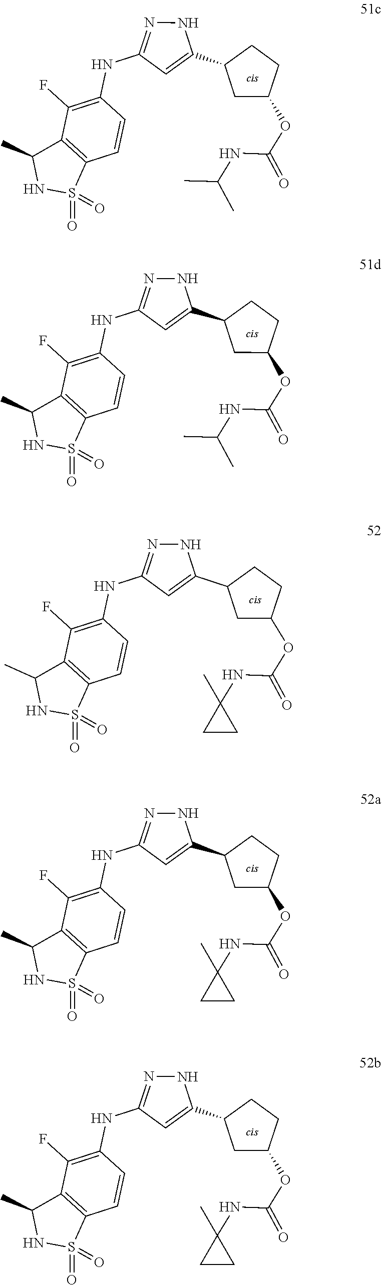 [SMILES: CC(C)NC(=O)O[C@H]1CC[C@@H](c2cc(Nc3ccc4c(c3F)[C@H](C)NS4(=O)=O)n[nH]2)C1 ; CC(C)NC(=O)O[C@@H]1CC[C@H](c2cc(Nc3ccc4c(c3F)[C@H](C)NS4(=O)=O)n[nH]2)C1 ; CC1NS(=O)(=O)c2ccc(Nc3cc(C4CCC(OC(=O)NC5(C)CC5)C4)[nH]n3)c(F)c21 ; C[C@@H]1NS(=O)(=O)c2ccc(Nc3cc([C@H]4CC[C@@H](OC(=O)NC5(C)CC5)C4)[nH]n3)c(F)c21 ; C[C@@H]1NS(=O)(=O)c2ccc(Nc3cc([C@@H]4CC[C@H](OC(=O)NC5(C)CC5)C4)[nH]n3)c(F)c21]

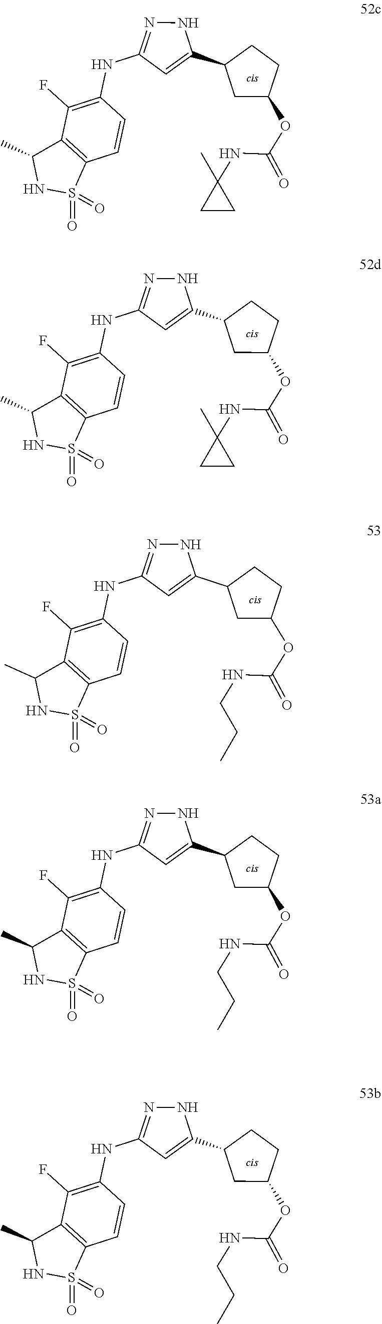 [SMILES: C[C@H]1NS(=O)(=O)c2ccc(Nc3cc([C@H]4CC[C@@H](OC(=O)NC5(C)CC5)C4)[nH]n3)c(F)c21 ; C[C@H]1NS(=O)(=O)c2ccc(Nc3cc([C@@H]4CC[C@H](OC(=O)NC5(C)CC5)C4)[nH]n3)c(F)c21 ; CCCNC(=O)OC1CCC(c2cc(Nc3ccc4c(c3F)C(C)NS4(=O)=O)n[nH]2)C1 ; CCCNC(=O)O[C@@H]1CC[C@H](c2cc(Nc3ccc4c(c3F)[C@H](C)NS4(=O)=O)n[nH]2)C1 ; CCCNC(=O)O[C@H]1CC[C@@H](c2cc(Nc3ccc4c(c3F)[C@H](C)NS4(=O)=O)n[nH]2)C1]

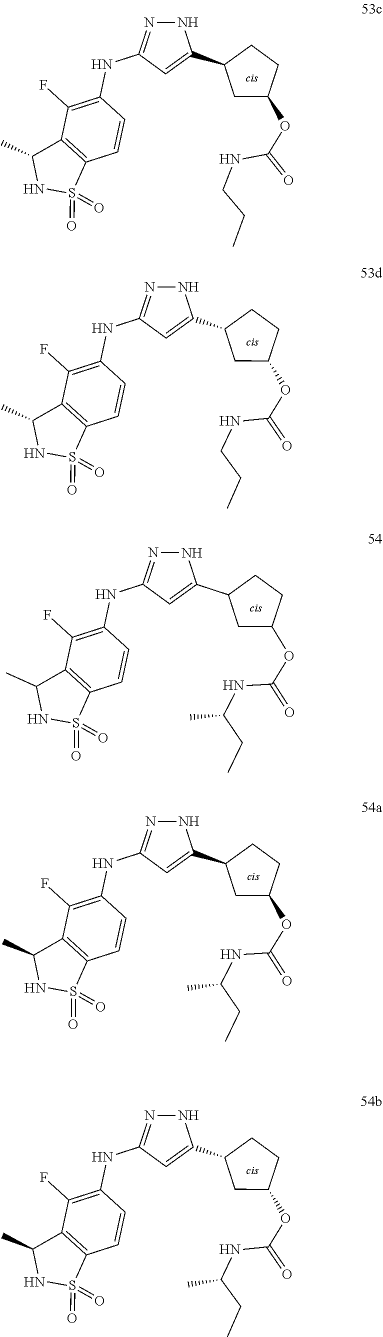 [SMILES: CCCNC(=O)O[C@@H]1CC[C@H](c2cc(Nc3ccc4c(c3F)[C@@H](C)NS4(=O)=O)n[nH]2)C1 ; CCCNC(=O)O[C@H]1CC[C@@H](c2cc(Nc3ccc4c(c3F)[C@@H](C)NS4(=O)=O)n[nH]2)C1 ; CC[C@H](C)NC(=O)OC1CCC(c2cc(Nc3ccc4c(c3F)C(C)NS4(=O)=O)n[nH]2)C1 ; CC[C@H](C)NC(=O)O[C@@H]1CC[C@H](c2cc(Nc3ccc4c(c3F)[C@H](C)NS4(=O)=O)n[nH]2)C1 ; CC[C@H](C)NC(=O)O[C@H]1CC[C@@H](c2cc(Nc3ccc4c(c3F)[C@H](C)NS4(=O)=O)n[nH]2)C1]

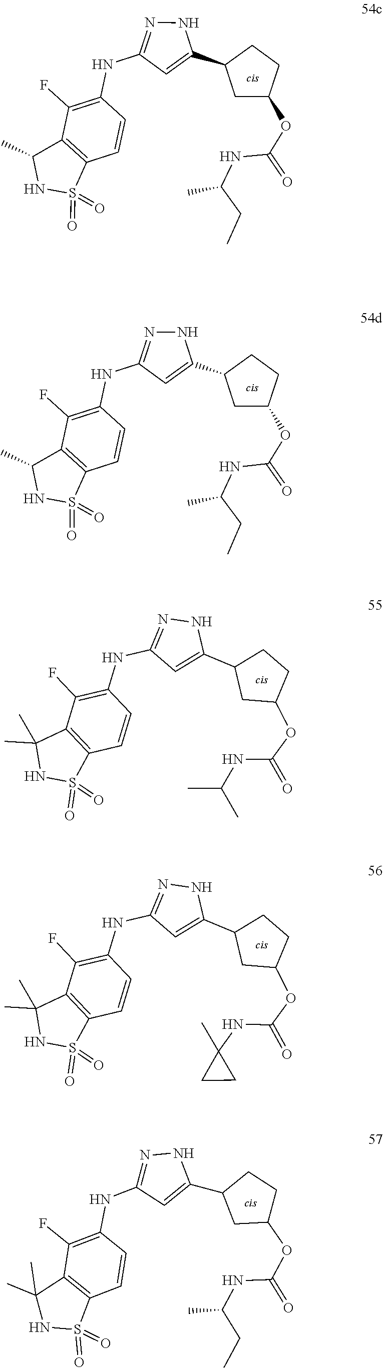 [SMILES: CC[C@H](C)NC(=O)O[C@@H]1CC[C@H](c2cc(Nc3ccc4c(c3F)[C@@H](C)NS4(=O)=O)n[nH]2)C1 ; CC[C@H](C)NC(=O)O[C@H]1CC[C@@H](c2cc(Nc3ccc4c(c3F)[C@@H](C)NS4(=O)=O)n[nH]2)C1 ; CC(C)NC(=O)OC1CCC(c2cc(Nc3ccc4c(c3F)C(C)(C)NS4(=O)=O)n[nH]2)C1 ; CC1(NC(=O)OC2CCC(c3cc(Nc4ccc5c(c4F)C(C)(C)NS5(=O)=O)n[nH]3)C2)CC1 ; CC[C@H](C)NC(=O)OC1CCC(c2cc(Nc3ccc4c(c3F)C(C)(C)NS4(=O)=O)n[nH]2)C1]

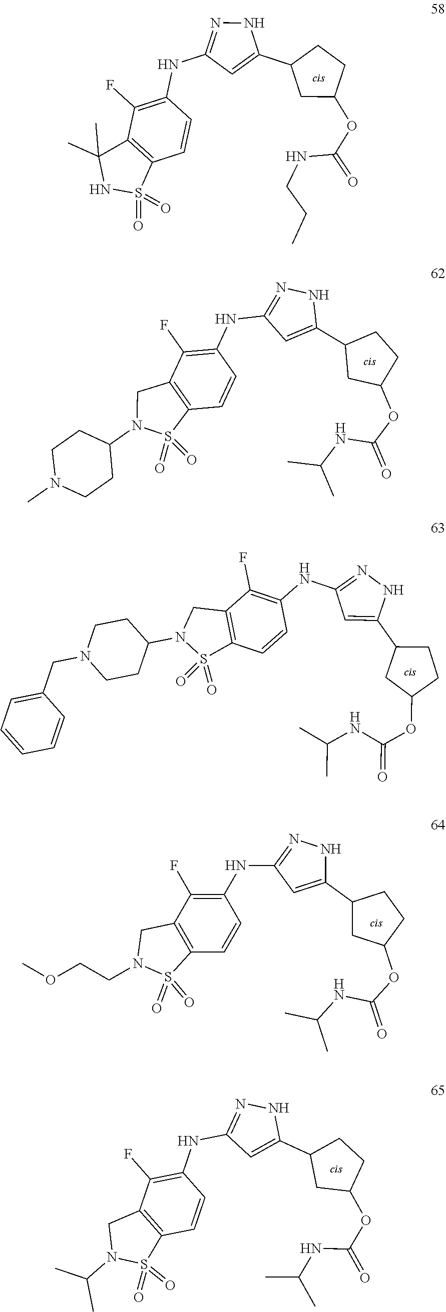

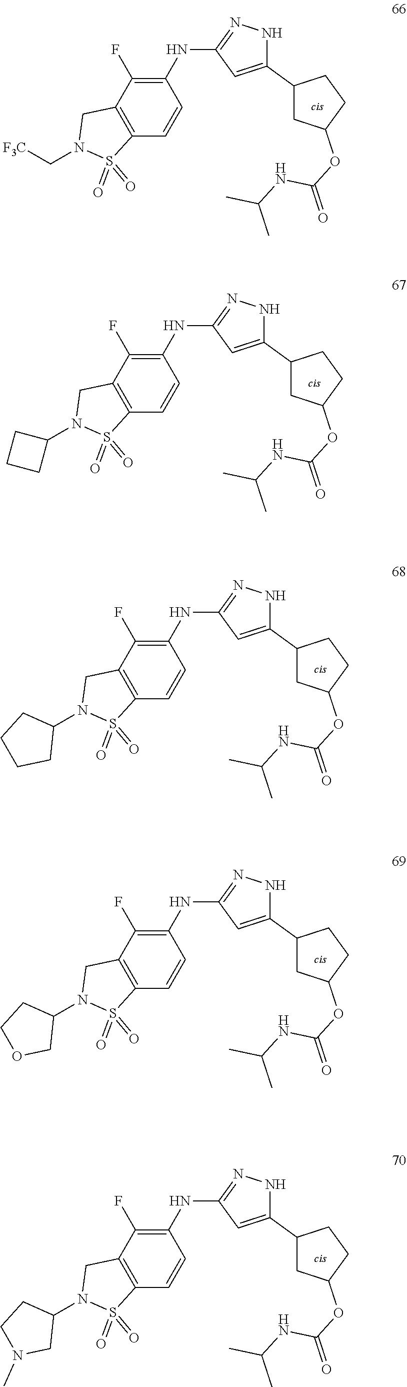 [SMILES: CC(C)NC(=O)OC1CCC(c2cc(Nc3ccc4c(c3F)CN(CC(F)(F)F)S4(=O)=O)n[nH]2)C1 ; CC(C)NC(=O)OC1CCC(c2cc(Nc3ccc4c(c3F)CN(C3CCC3)S4(=O)=O)n[nH]2)C1 ; CC(C)NC(=O)OC1CCC(c2cc(Nc3ccc4c(c3F)CN(C3CCCC3)S4(=O)=O)n[nH]2)C1 ; CC(C)NC(=O)OC1CCC(c2cc(Nc3ccc4c(c3F)CN(C3CCOC3)S4(=O)=O)n[nH]2)C1 ; CC(C)NC(=O)OC1CCC(c2cc(Nc3ccc4c(c3F)CN(C3CCN(C)C3)S4(=O)=O)n[nH]2)C1]

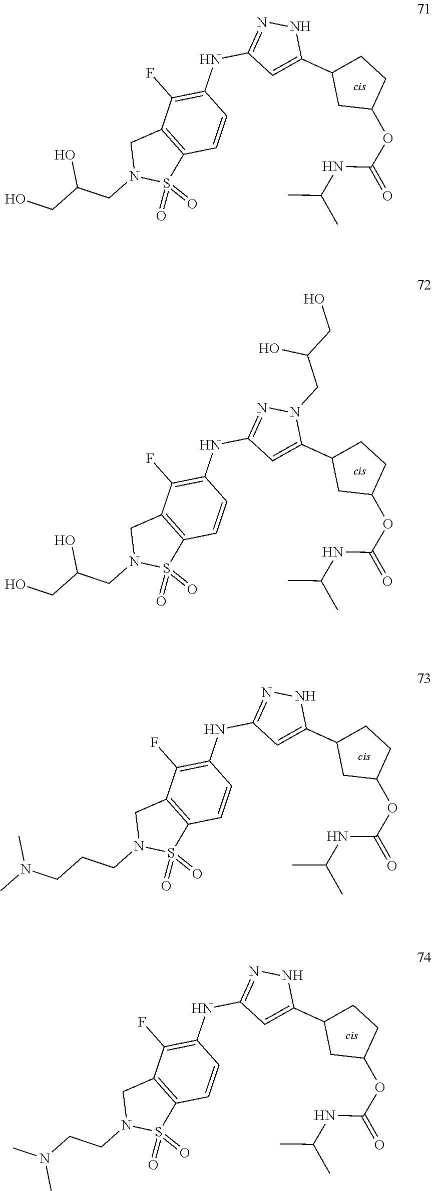 [SMILES: CC(C)NC(=O)OC1CCC(c2cc(Nc3ccc4c(c3F)CN(CC(O)CO)S4(=O)=O)n[nH]2)C1 ; CC(C)NC(=O)OC1CCC(c2cc(Nc3ccc4c(c3F)CN(CC(O)CO)S4(=O)=O)nn2CC(O)CO)C1 ; CC(C)NC(=O)OC1CCC(c2cc(Nc3ccc4c(c3F)CN(CCCN(C)C)S4(=O)=O)n[nH]2)C1 ; CC(C)NC(=O)OC1CCC(c2cc(Nc3ccc4c(c3F)CN(CCN(C)C)S4(=O)=O)n[nH]2)C1]

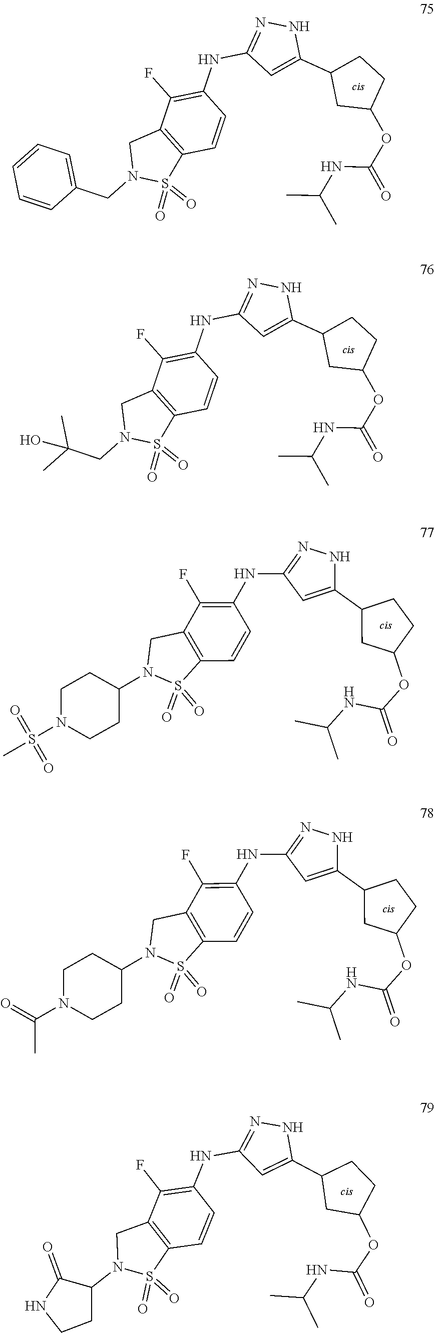 [SMILES: CC(C)NC(=O)OC1CCC(c2cc(Nc3ccc4c(c3F)CN(Cc3ccccc3)S4(=O)=O)n[nH]2)C1 ; CC(C)NC(=O)OC1CCC(c2cc(Nc3ccc4c(c3F)CN(CC(C)(C)O)S4(=O)=O)n[nH]2)C1 ; CC(C)NC(=O)OC1CCC(c2cc(Nc3ccc4c(c3F)CN(C3CCN(S(C)(=O)=O)CC3)S4(=O)=O)n[nH]2)C1 ; CC(=O)N1CCC(N2Cc3c(ccc(Nc4cc(C5CCC(OC(=O)NC(C)C)C5)[nH]n4)c3F)S2(=O)=O)CC1 ; CC(C)NC(=O)OC1CCC(c2cc(Nc3ccc4c(c3F)CN(C3CCNC3=O)S4(=O)=O)n[nH]2)C1]

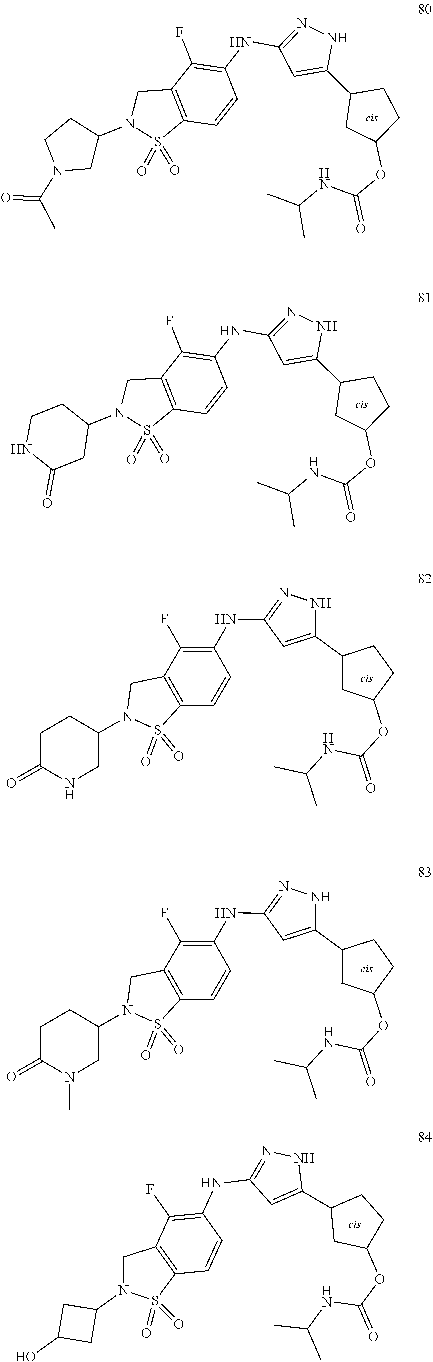 [SMILES: CC(=O)N1CCC(N2Cc3c(ccc(Nc4cc(C5CCC(OC(=O)NC(C)C)C5)[nH]n4)c3F)S2(=O)=O)C1 ; CC(C)NC(=O)OC1CCC(c2cc(Nc3ccc4c(c3F)CN(C3CCNC(=O)C3)S4(=O)=O)n[nH]2)C1 ; CC(C)NC(=O)OC1CCC(c2cc(Nc3ccc4c(c3F)CN(C3CCC(=O)NC3)S4(=O)=O)n[nH]2)C1 ; CC(C)NC(=O)OC1CCC(c2cc(Nc3ccc4c(c3F)CN(C3CCC(=O)N(C)C3)S4(=O)=O)n[nH]2)C1 ; CC(C)NC(=O)OC1CCC(c2cc(Nc3ccc4c(c3F)CN(C3CC(O)C3)S4(=O)=O)n[nH]2)C1]

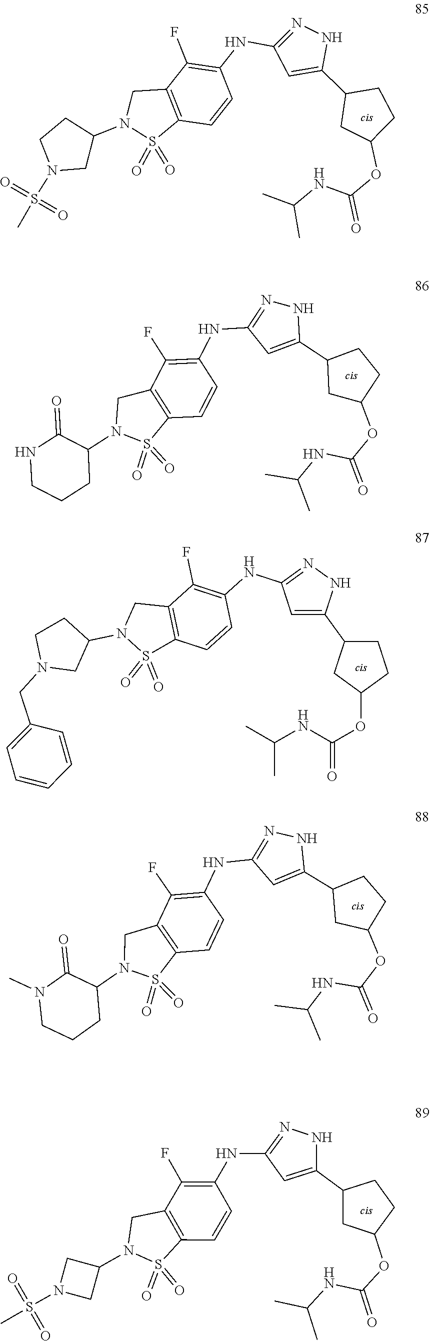 [SMILES: CC(C)NC(=O)OC1CCC(c2cc(Nc3ccc4c(c3F)CN(C3CCN(S(C)(=O)=O)C3)S4(=O)=O)n[nH]2)C1 ; CC(C)NC(=O)OC1CCC(c2cc(Nc3ccc4c(c3F)CN(C3CCCNC3=O)S4(=O)=O)n[nH]2)C1 ; CC(C)NC(=O)OC1CCC(c2cc(Nc3ccc4c(c3F)CN(C3CCN(Cc5ccccc5)C3)S4(=O)=O)n[nH]2)C1 ; CC(C)NC(=O)OC1CCC(c2cc(Nc3ccc4c(c3F)CN(C3CCCN(C)C3=O)S4(=O)=O)n[nH]2)C1 ; CC(C)NC(=O)OC1CCC(c2cc(Nc3ccc4c(c3F)CN(C3CN(S(C)(=O)=O)C3)S4(=O)=O)n[nH]2)C1]

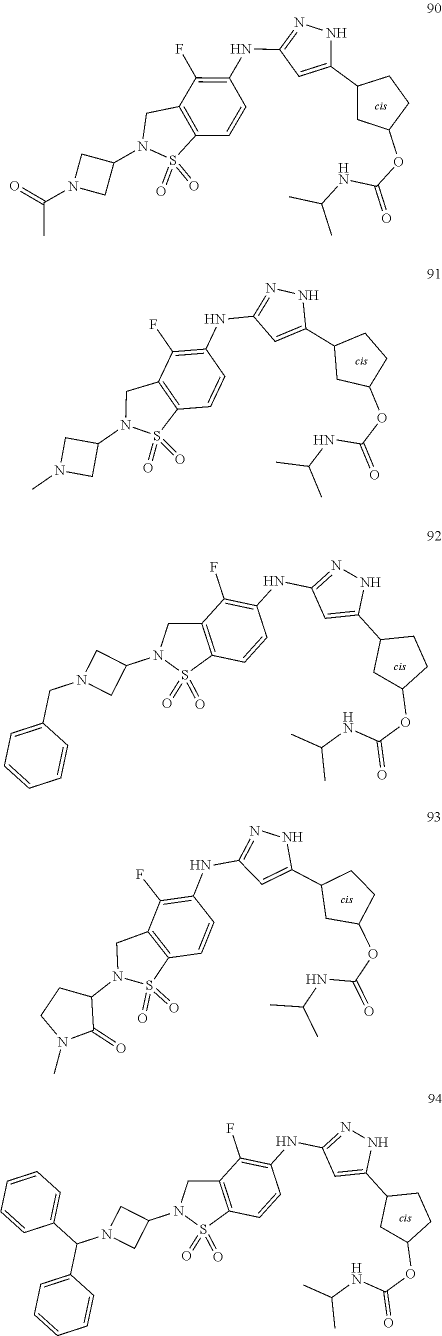 [SMILES: CC(=O)N1CC(N2Cc3c(ccc(Nc4cc(C5CCC(OC(=O)NC(C)C)C5)[nH]n4)c3F)S2(=O)=O)C1 ; CC(C)NC(=O)OC1CCC(c2cc(Nc3ccc4c(c3F)CN(C3CN(C)C3)S4(=O)=O)n[nH]2)C1 ; CC(C)NC(=O)OC1CCC(c2cc(Nc3ccc4c(c3F)CN(C3CN(Cc5ccccc5)C3)S4(=O)=O)n[nH]2)C1 ; CC(C)NC(=O)OC1CCC(c2cc(Nc3ccc4c(c3F)CN(C3CCN(C)C3=O)S4(=O)=O)n[nH]2)C1 ; CC(C)NC(=O)OC1CCC(c2cc(Nc3ccc4c(c3F)CN(C3CN(C(c5ccccc5)c5ccccc5)C3)S4(=O)=O)n[nH]2)C1]

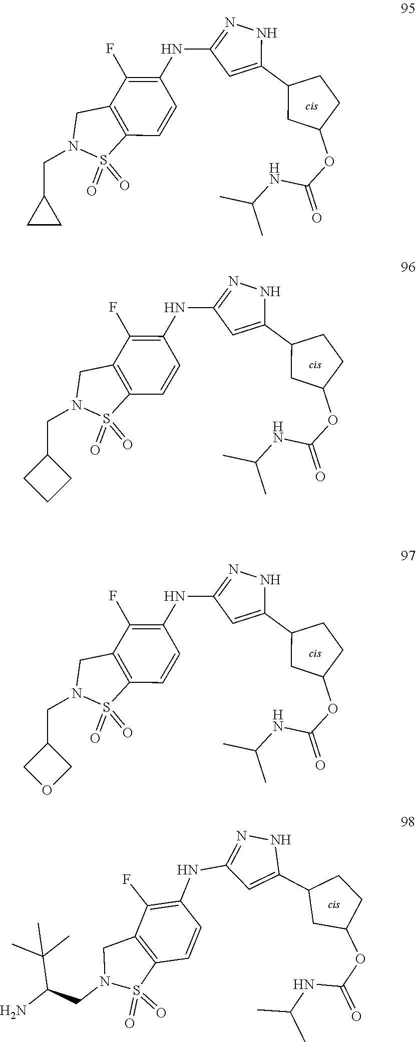 [SMILES: CC(C)NC(=O)OC1CCC(c2cc(Nc3ccc4c(c3F)CN(CC3CC3)S4(=O)=O)n[nH]2)C1 ; CC(C)NC(=O)OC1CCC(c2cc(Nc3ccc4c(c3F)CN(CC3CCC3)S4(=O)=O)n[nH]2)C1 ; CC(C)NC(=O)OC1CCC(c2cc(Nc3ccc4c(c3F)CN(CC3COC3)S4(=O)=O)n[nH]2)C1 ; CC(C)NC(=O)OC1CCC(c2cc(Nc3ccc4c(c3F)CN(C[C@@H](N)C(C)(C)C)S4(=O)=O)n[nH]2)C1]

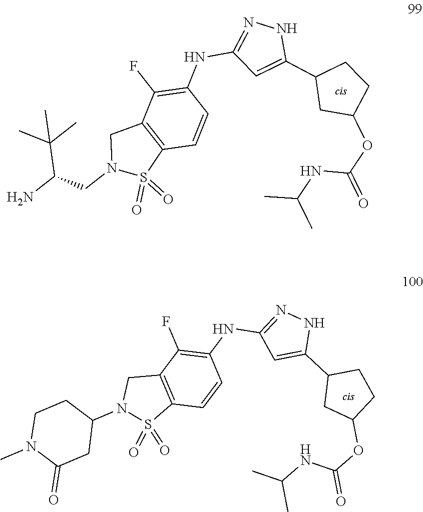 [SMILES: CC(C)NC(=O)OC1CCC(c2cc(Nc3ccc4c(c3F)CN(C[C@H](N)C(C)(C)C)S4(=O)=O)n[nH]2)C1 ; CC(C)NC(=O)OC1CCC(c2cc(Nc3ccc4c(c3F)CN(C3CCN(C)C(=O)C3)S4(=O)=O)n[nH]2)C1]

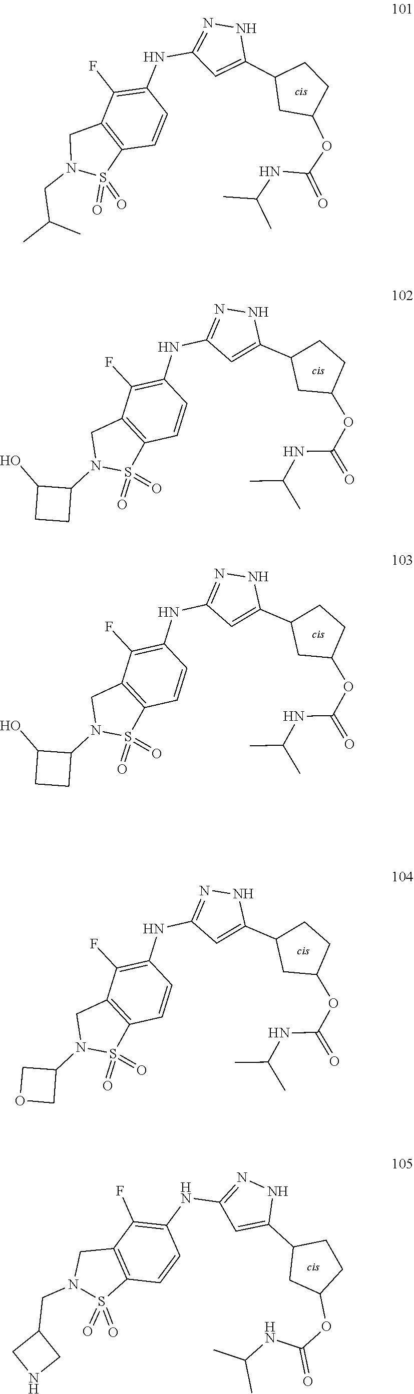 [SMILES: CC(C)CN1Cc2c(ccc(Nc3cc(C4CCC(OC(=O)NC(C)C)C4)[nH]n3)c2F)S1(=O)=O ; CC(C)NC(=O)OC1CCC(c2cc(Nc3ccc4c(c3F)CN(C3CCC3O)S4(=O)=O)n[nH]2)C1 ; CC(C)NC(=O)OC1CCC(c2cc(Nc3ccc4c(c3F)CN(C3CCC3O)S4(=O)=O)n[nH]2)C1 ; CC(C)NC(=O)OC1CCC(c2cc(Nc3ccc4c(c3F)CN(C3COC3)S4(=O)=O)n[nH]2)C1 ; CC(C)NC(=O)OC1CCC(c2cc(Nc3ccc4c(c3F)CN(CC3CNC3)S4(=O)=O)n[nH]2)C1]

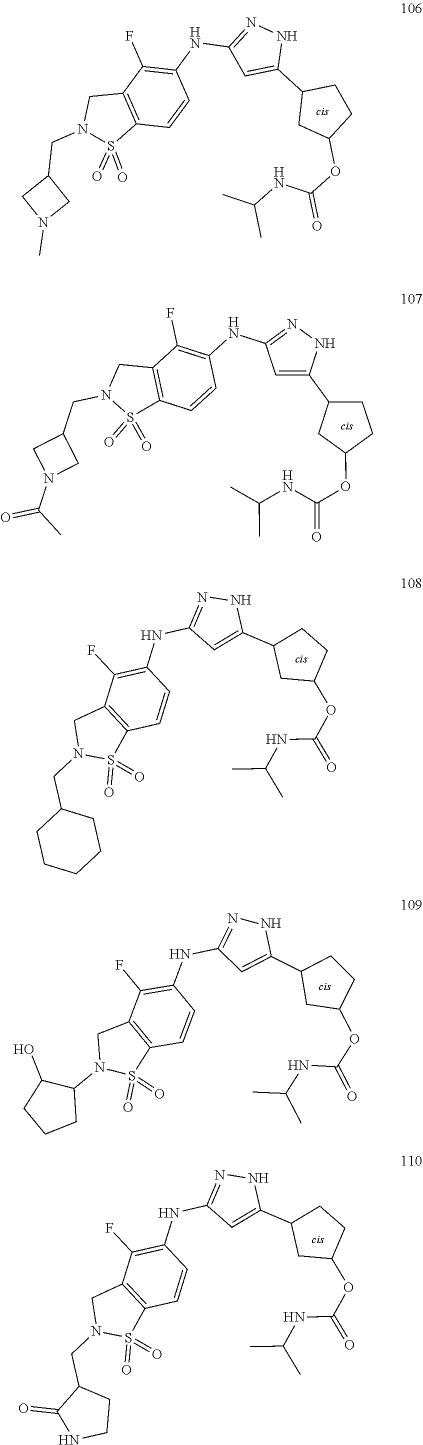 [SMILES: CC(C)NC(=O)OC1CCC(c2cc(Nc3ccc4c(c3F)CN(CC3CN(C)C3)S4(=O)=O)n[nH]2)C1 ; CC(=O)N1CC(CN2Cc3c(ccc(Nc4cc(C5CCC(OC(=O)NC(C)C)C5)[nH]n4)c3F)S2(=O)=O)C1 ; CC(C)NC(=O)OC1CCC(c2cc(Nc3ccc4c(c3F)CN(CC3CCCCC3)S4(=O)=O)n[nH]2)C1 ; CC(C)NC(=O)OC1CCC(c2cc(Nc3ccc4c(c3F)CN(C3CCCC3O)S4(=O)=O)n[nH]2)C1 ; CC(C)NC(=O)OC1CCC(c2cc(Nc3ccc4c(c3F)CN(CC3CCNC3=O)S4(=O)=O)n[nH]2)C1]

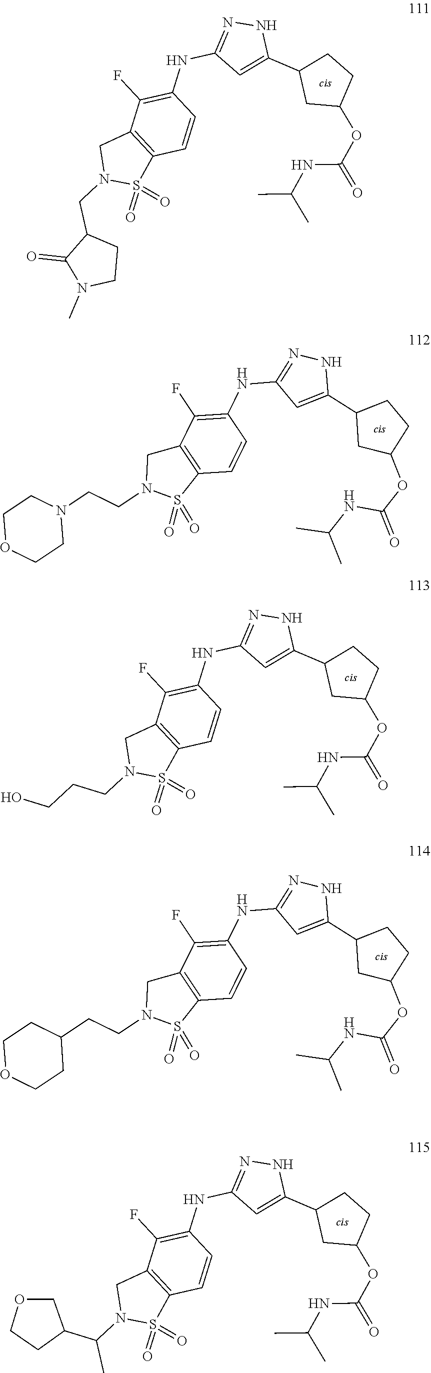 [SMILES: CC(C)NC(=O)OC1CCC(c2cc(Nc3ccc4c(c3F)CN(CC3CCN(C)C3=O)S4(=O)=O)n[nH]2)C1 ; CC(C)NC(=O)OC1CCC(c2cc(Nc3ccc4c(c3F)CN(CCN3CCOCC3)S4(=O)=O)n[nH]2)C1 ; CC(C)NC(=O)OC1CCC(c2cc(Nc3ccc4c(c3F)CN(CCCO)S4(=O)=O)n[nH]2)C1 ; CC(C)NC(=O)OC1CCC(c2cc(Nc3ccc4c(c3F)CN(CCC3CCOCC3)S4(=O)=O)n[nH]2)C1 ; CC(C)NC(=O)OC1CCC(c2cc(Nc3ccc4c(c3F)CN(C(C)C3CCOC3)S4(=O)=O)n[nH]2)C1]

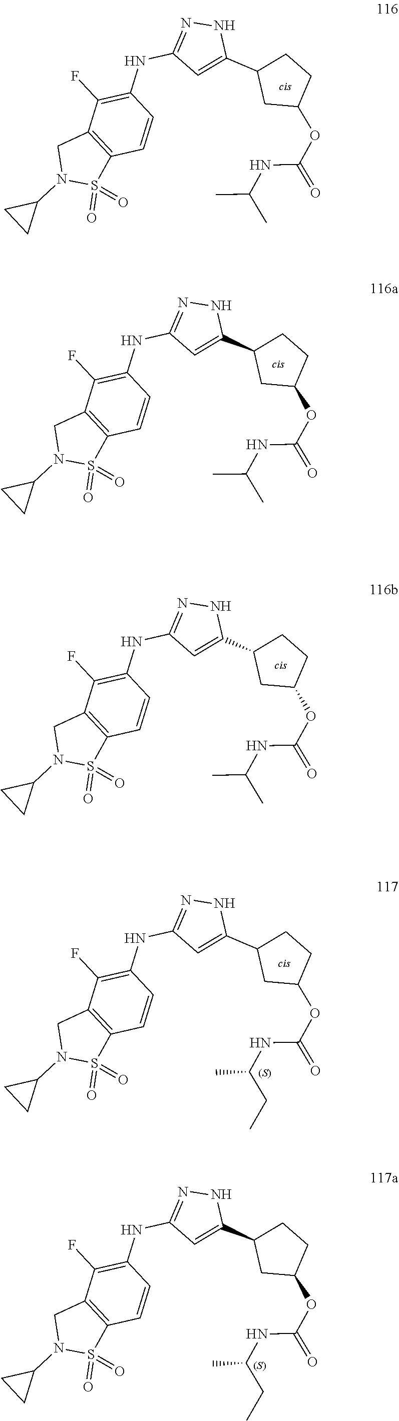

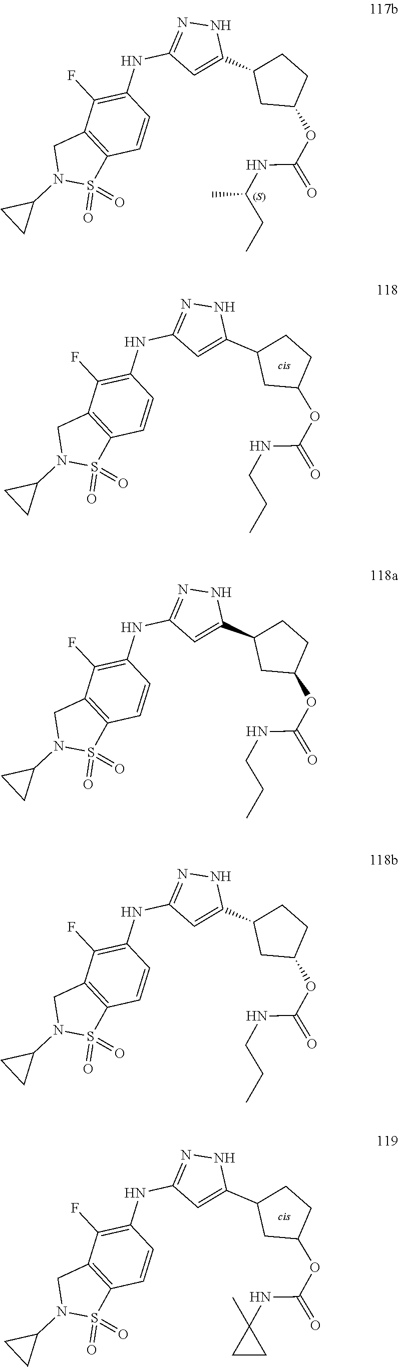

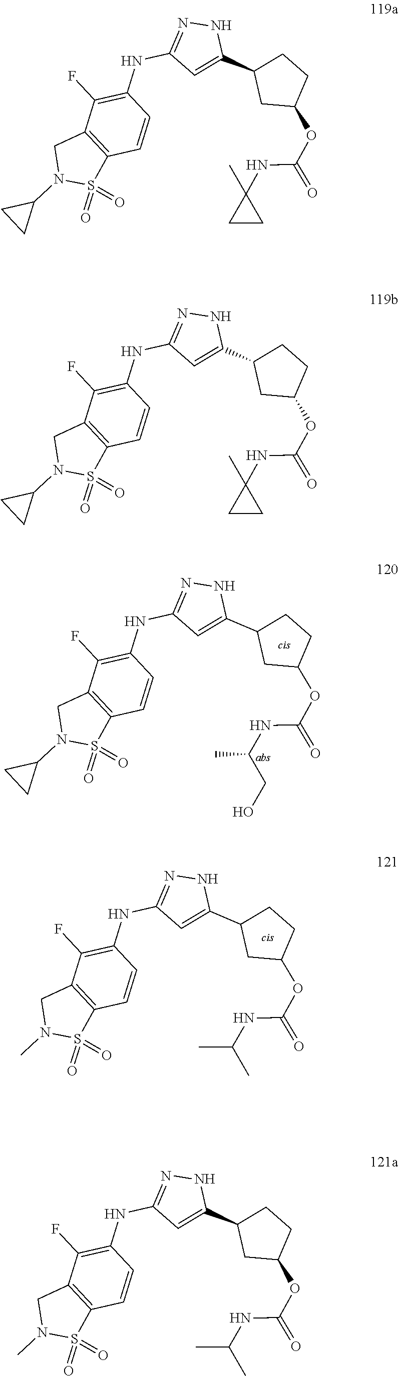 [SMILES: CC1(NC(=O)O[C@@H]2CC[C@H](c3cc(Nc4ccc5c(c4F)CN(C4CC4)S5(=O)=O)n[nH]3)C2)CC1 ; CC1(NC(=O)O[C@H]2CC[C@@H](c3cc(Nc4ccc5c(c4F)CN(C4CC4)S5(=O)=O)n[nH]3)C2)CC1 ; C[C@@H](CO)NC(=O)OC1CCC(c2cc(Nc3ccc4c(c3F)CN(C3CC3)S4(=O)=O)n[nH]2)C1 ; CC(C)NC(=O)OC1CCC(c2cc(Nc3ccc4c(c3F)CN(C)S4(=O)=O)n[nH]2)C1 ; CC(C)NC(=O)O[C@@H]1CC[C@H](c2cc(Nc3ccc4c(c3F)CN(C)S4(=O)=O)n[nH]2)C1]

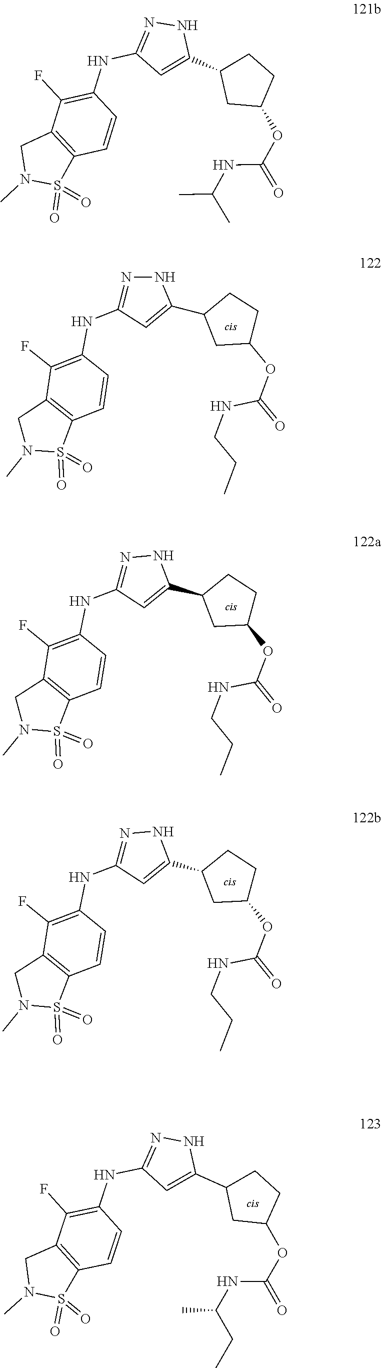 [SMILES: CC(C)NC(=O)O[C@H]1CC[C@@H](c2cc(Nc3ccc4c(c3F)CN(C)S4(=O)=O)n[nH]2)C1 ; CCCNC(=O)OC1CCC(c2cc(Nc3ccc4c(c3F)CN(C)S4(=O)=O)n[nH]2)C1 ; CCCNC(=O)O[C@@H]1CC[C@H](c2cc(Nc3ccc4c(c3F)CN(C)S4(=O)=O)n[nH]2)C1 ; CCCNC(=O)O[C@H]1CC[C@@H](c2cc(Nc3ccc4c(c3F)CN(C)S4(=O)=O)n[nH]2)C1 ; CC[C@H](C)NC(=O)OC1CCC(c2cc(Nc3ccc4c(c3F)CN(C)S4(=O)=O)n[nH]2)C1]

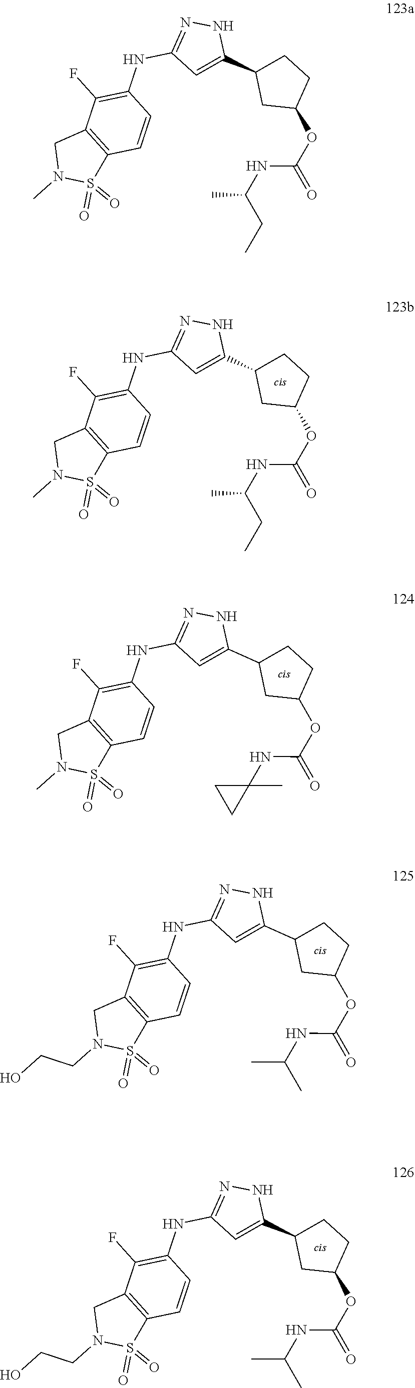 [SMILES: CC[C@H](C)NC(=O)O[C@@H]1CC[C@H](c2cc(Nc3ccc4c(c3F)CN(C)S4(=O)=O)n[nH]2)C1 ; CC[C@H](C)NC(=O)O[C@H]1CC[C@@H](c2cc(Nc3ccc4c(c3F)CN(C)S4(=O)=O)n[nH]2)C1 ; CN1Cc2c(ccc(Nc3cc(C4CCC(OC(=O)NC5(C)CC5)C4)[nH]n3)c2F)S1(=O)=O ; CC(C)NC(=O)OC1CCC(c2cc(Nc3ccc4c(c3F)CN(CCO)S4(=O)=O)n[nH]2)C1 ; CC(C)NC(=O)O[C@@H]1CC[C@H](c2cc(Nc3ccc4c(c3F)CN(CCO)S4(=O)=O)n[nH]2)C1]

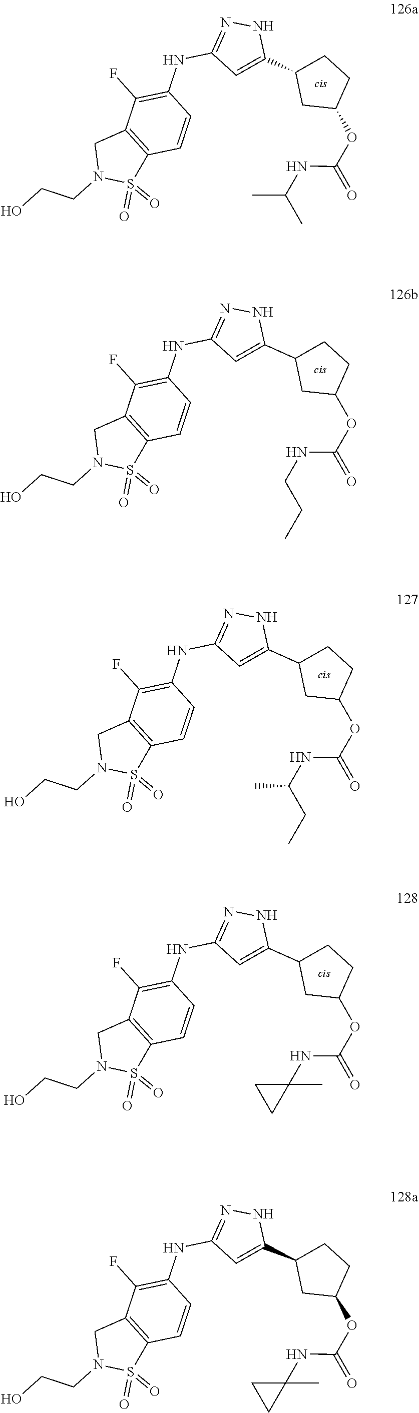 [SMILES: CC(C)NC(=O)O[C@H]1CC[C@@H](c2cc(Nc3ccc4c(c3F)CN(CCO)S4(=O)=O)n[nH]2)C1 ; CCCNC(=O)OC1CCC(c2cc(Nc3ccc4c(c3F)CN(CCO)S4(=O)=O)n[nH]2)C1 ; CC[C@H](C)NC(=O)OC1CCC(c2cc(Nc3ccc4c(c3F)CN(CCO)S4(=O)=O)n[nH]2)C1 ; CC1(NC(=O)OC2CCC(c3cc(Nc4ccc5c(c4F)CN(CCO)S5(=O)=O)n[nH]3)C2)CC1 ; CC1(NC(=O)O[C@@H]2CC[C@H](c3cc(Nc4ccc5c(c4F)CN(CCO)S5(=O)=O)n[nH]3)C2)CC1]

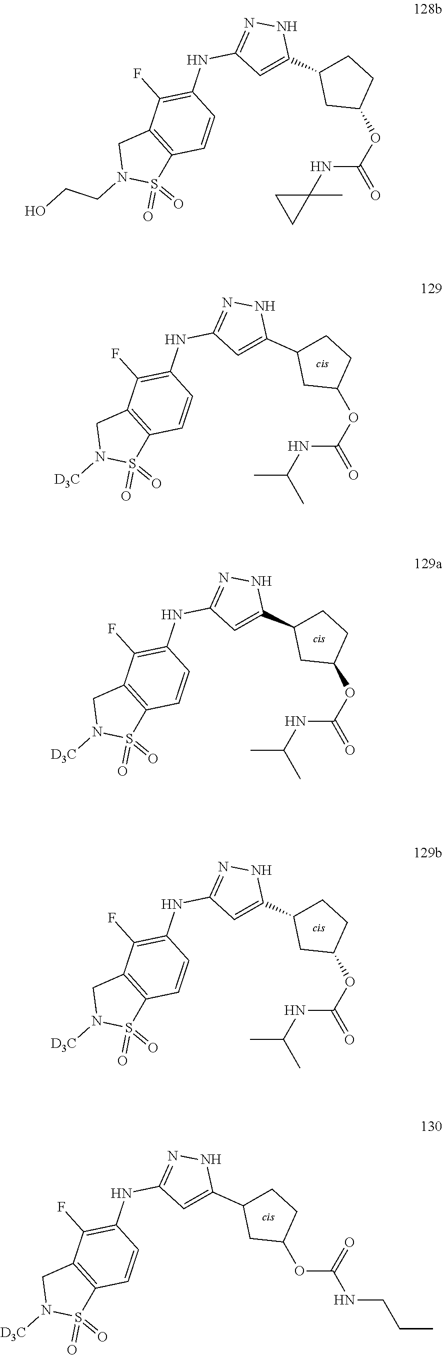 [SMILES: CC1(NC(=O)O[C@H]2CC[C@@H](c3cc(Nc4ccc5c(c4F)CN(CCO)S5(=O)=O)n[nH]3)C2)CC1 ; [2H]C([2H])([2H])N1Cc2c(ccc(Nc3cc(C4CCC(OC(=O)NC(C)C)C4)[nH]n3)c2F)S1(=O)=O ; [2H]C([2H])([2H])N1Cc2c(ccc(Nc3cc([C@H]4CC[C@@H](OC(=O)NC(C)C)C4)[nH]n3)c2F)S1(=O)=O ; [2H]C([2H])([2H])N1Cc2c(ccc(Nc3cc([C@@H]4CC[C@H](OC(=O)NC(C)C)C4)[nH]n3)c2F)S1(=O)=O ; [2H]C([2H])([2H])N1Cc2c(ccc(Nc3cc(C4CCC(OC(=O)NCCC)C4)[nH]n3)c2F)S1(=O)=O]

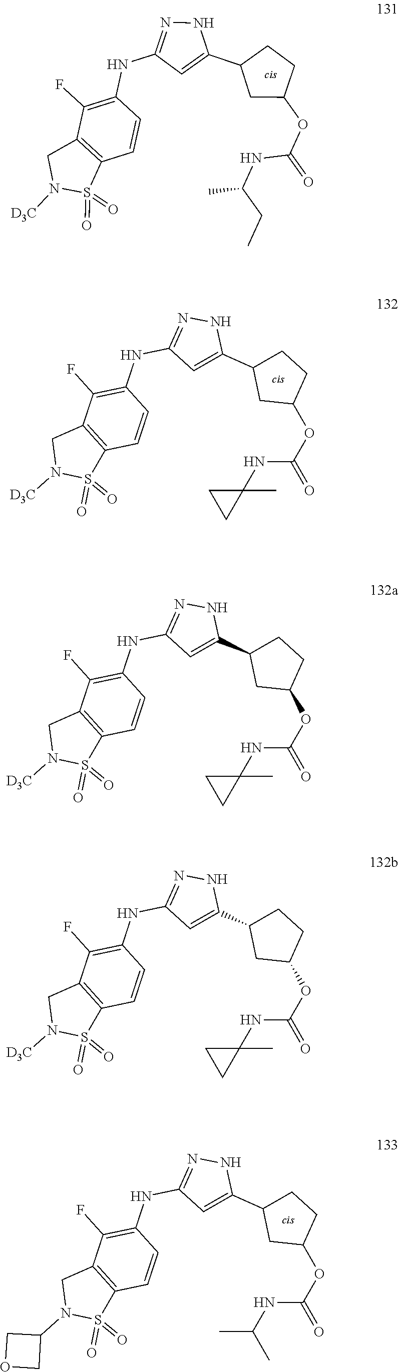 [SMILES: [2H]C([2H])([2H])N1Cc2c(ccc(Nc3cc(C4CCC(OC(=O)N[C@@H](C)CC)C4)[nH]n3)c2F)S1(=O)=O ; [2H]C([2H])([2H])N1Cc2c(ccc(Nc3cc(C4CCC(OC(=O)NC5(C)CC5)C4)[nH]n3)c2F)S1(=O)=O ; [2H]C([2H])([2H])N1Cc2c(ccc(Nc3cc([C@H]4CC[C@@H](OC(=O)NC5(C)CC5)C4)[nH]n3)c2F)S1(=O)=O ; [2H]C([2H])([2H])N1Cc2c(ccc(Nc3cc([C@@H]4CC[C@H](OC(=O)NC5(C)CC5)C4)[nH]n3)c2F)S1(=O)=O ; CC(C)NC(=O)OC1CCC(c2cc(Nc3ccc4c(c3F)CN(C3COC3)S4(=O)=O)n[nH]2)C1]

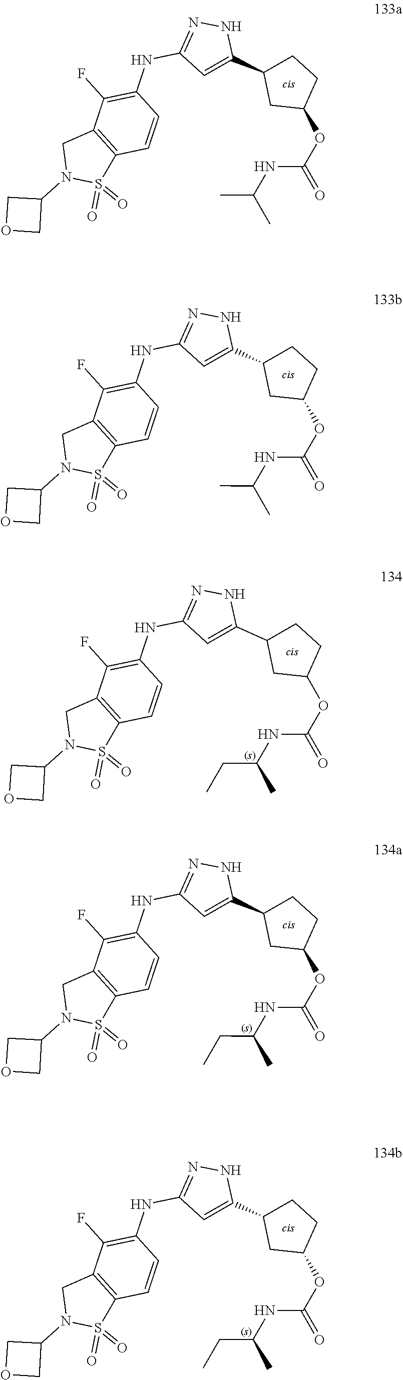 [SMILES: CC(C)NC(=O)O[C@@H]1CC[C@H](c2cc(Nc3ccc4c(c3F)CN(C3COC3)S4(=O)=O)n[nH]2)C1 ; CC(C)NC(=O)O[C@H]1CC[C@@H](c2cc(Nc3ccc4c(c3F)CN(C3COC3)S4(=O)=O)n[nH]2)C1 ; CC[C@H](C)NC(=O)OC1CCC(c2cc(Nc3ccc4c(c3F)CN(C3COC3)S4(=O)=O)n[nH]2)C1 ; CC[C@H](C)NC(=O)O[C@@H]1CC[C@H](c2cc(Nc3ccc4c(c3F)CN(C3COC3)S4(=O)=O)n[nH]2)C1 ; CC[C@H](C)NC(=O)O[C@H]1CC[C@@H](c2cc(Nc3ccc4c(c3F)CN(C3COC3)S4(=O)=O)n[nH]2)C1]

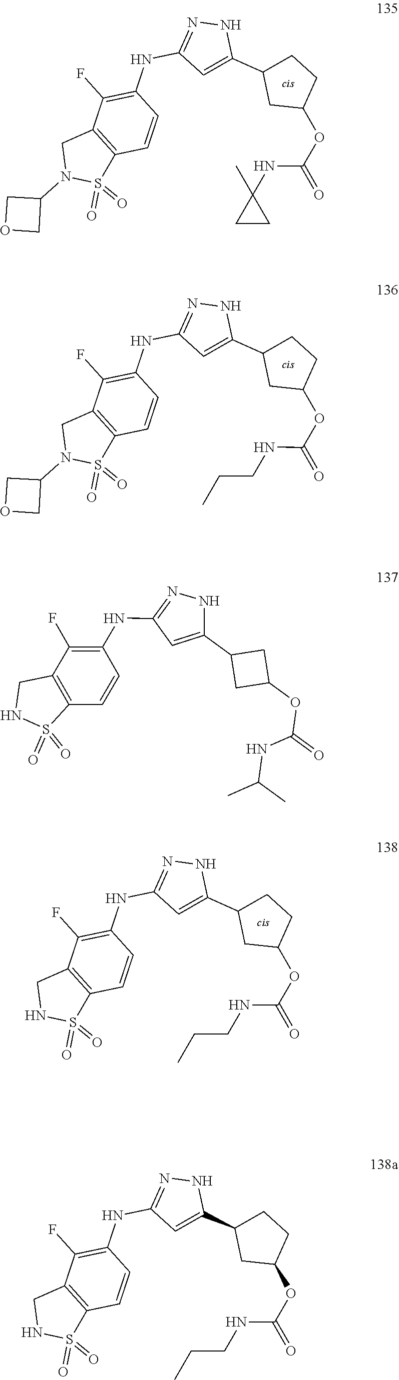 [SMILES: CC1(NC(=O)OC2CCC(c3cc(Nc4ccc5c(c4F)CN(C4COC4)S5(=O)=O)n[nH]3)C2)CC1 ; CCCNC(=O)OC1CCC(c2cc(Nc3ccc4c(c3F)CN(C3COC3)S4(=O)=O)n[nH]2)C1 ; CC(C)NC(=O)OC1CC(c2cc(Nc3ccc4c(c3F)CNS4(=O)=O)n[nH]2)C1 ; CCCNC(=O)OC1CCC(c2cc(Nc3ccc4c(c3F)CNS4(=O)=O)n[nH]2)C1 ; CCCNC(=O)O[C@@H]1CC[C@H](c2cc(Nc3ccc4c(c3F)CNS4(=O)=O)n[nH]2)C1]

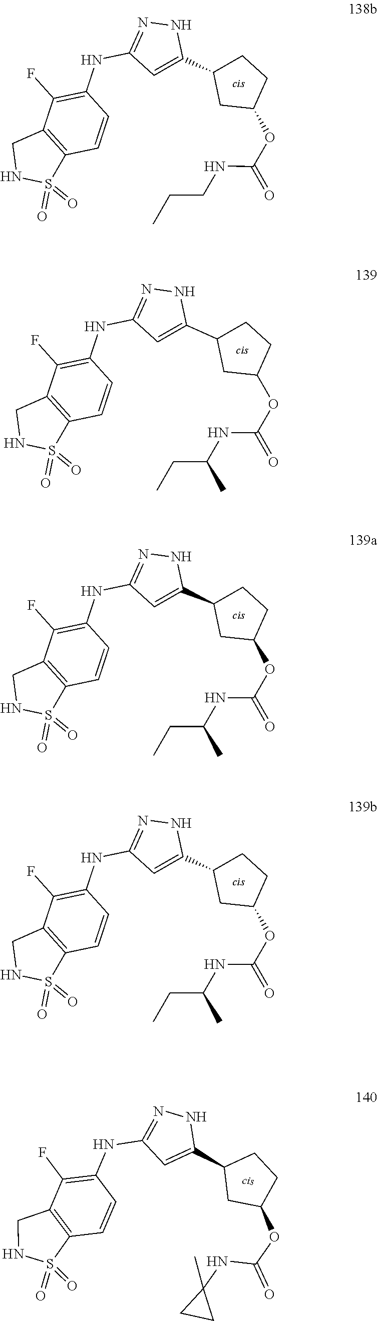 [SMILES: CCCNC(=O)O[C@H]1CC[C@@H](c2cc(Nc3ccc4c(c3F)CNS4(=O)=O)n[nH]2)C1 ; CC[C@H](C)NC(=O)OC1CCC(c2cc(Nc3ccc4c(c3F)CNS4(=O)=O)n[nH]2)C1 ; CC[C@H](C)NC(=O)O[C@@H]1CC[C@H](c2cc(Nc3ccc4c(c3F)CNS4(=O)=O)n[nH]2)C1 ; CC[C@H](C)NC(=O)O[C@H]1CC[C@@H](c2cc(Nc3ccc4c(c3F)CNS4(=O)=O)n[nH]2)C1 ; CC1(NC(=O)O[C@@H]2CC[C@H](c3cc(Nc4ccc5c(c4F)CNS5(=O)=O)n[nH]3)C2)CC1]

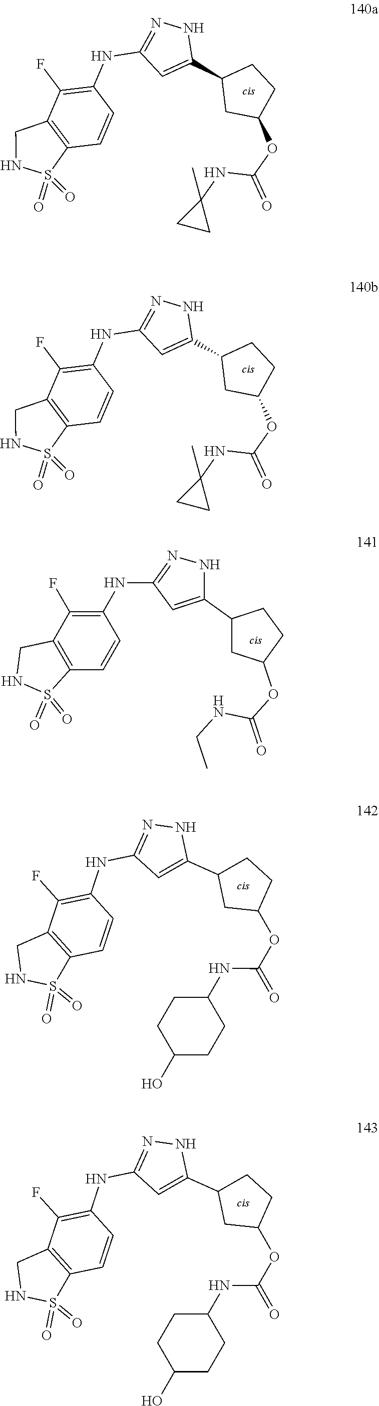 [SMILES: CC1(NC(=O)O[C@@H]2CC[C@H](c3cc(Nc4ccc5c(c4F)CNS5(=O)=O)n[nH]3)C2)CC1 ; CC1(NC(=O)O[C@H]2CC[C@@H](c3cc(Nc4ccc5c(c4F)CNS5(=O)=O)n[nH]3)C2)CC1 ; CCNC(=O)OC1CCC(c2cc(Nc3ccc4c(c3F)CNS4(=O)=O)n[nH]2)C1 ; O=C(NC1CCC(O)CC1)OC1CCC(c2cc(Nc3ccc4c(c3F)CNS4(=O)=O)n[nH]2)C1 ; O=C(NC1CCC(O)CC1)OC1CCC(c2cc(Nc3ccc4c(c3F)CNS4(=O)=O)n[nH]2)C1]

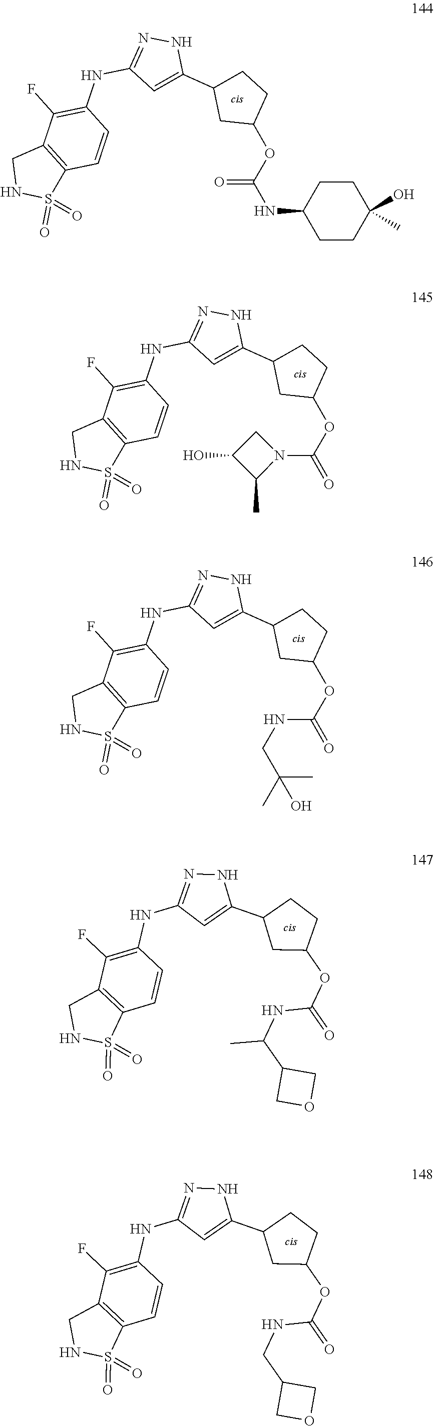 [SMILES: C[C@]1(O)CC[C@@H](NC(=O)OC2CCC(c3cc(Nc4ccc5c(c4F)CNS5(=O)=O)n[nH]3)C2)CC1 ; C[C@H]1[C@H](O)CN1C(=O)OC1CCC(c2cc(Nc3ccc4c(c3F)CNS4(=O)=O)n[nH]2)C1 ; CC(C)(O)CNC(=O)OC1CCC(c2cc(Nc3ccc4c(c3F)CNS4(=O)=O)n[nH]2)C1 ; CC(NC(=O)OC1CCC(c2cc(Nc3ccc4c(c3F)CNS4(=O)=O)n[nH]2)C1)C1COC1 ; O=C(NCC1COC1)OC1CCC(c2cc(Nc3ccc4c(c3F)CNS4(=O)=O)n[nH]2)C1]

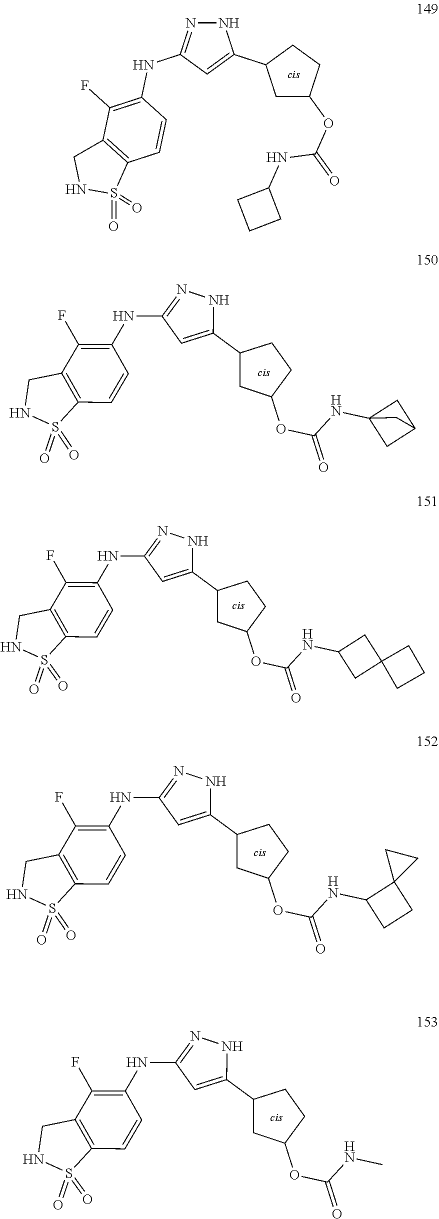 [SMILES: O=C(NC1CCC1)OC1CCC(c2cc(Nc3ccc4c(c3F)CNS4(=O)=O)n[nH]2)C1 ; O=C(NC12CC(C1)C2)OC1CCC(c2cc(Nc3ccc4c(c3F)CNS4(=O)=O)n[nH]2)C1 ; O=C(NC1CC2(CCC2)C1)OC1CCC(c2cc(Nc3ccc4c(c3F)CNS4(=O)=O)n[nH]2)C1 ; O=C(NC1CCC12CC2)OC1CCC(c2cc(Nc3ccc4c(c3F)CNS4(=O)=O)n[nH]2)C1 ; CNC(=O)OC1CCC(c2cc(Nc3ccc4c(c3F)CNS4(=O)=O)n[nH]2)C1]

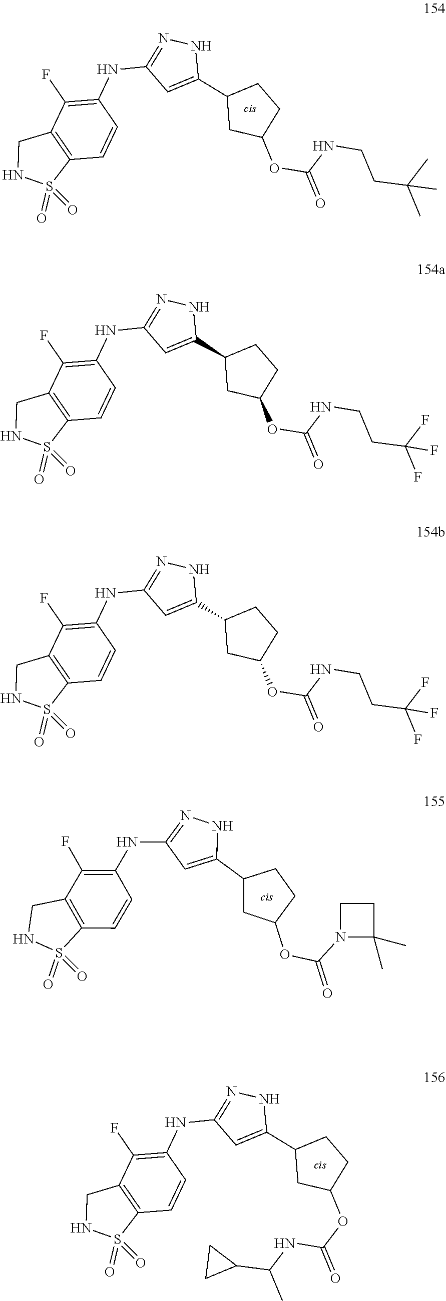 [SMILES: CC(C)(C)CCNC(=O)OC1CCC(c2cc(Nc3ccc4c(c3F)CNS4(=O)=O)n[nH]2)C1 ; O=C(NCCC(F)(F)F)O[C@@H]1CC[C@H](c2cc(Nc3ccc4c(c3F)CNS4(=O)=O)n[nH]2)C1 ; O=C(NCCC(F)(F)F)O[C@H]1CC[C@@H](c2cc(Nc3ccc4c(c3F)CNS4(=O)=O)n[nH]2)C1 ; CC1(C)CCN1C(=O)OC1CCC(c2cc(Nc3ccc4c(c3F)CNS4(=O)=O)n[nH]2)C1 ; CC(NC(=O)OC1CCC(c2cc(Nc3ccc4c(c3F)CNS4(=O)=O)n[nH]2)C1)C1CC1]

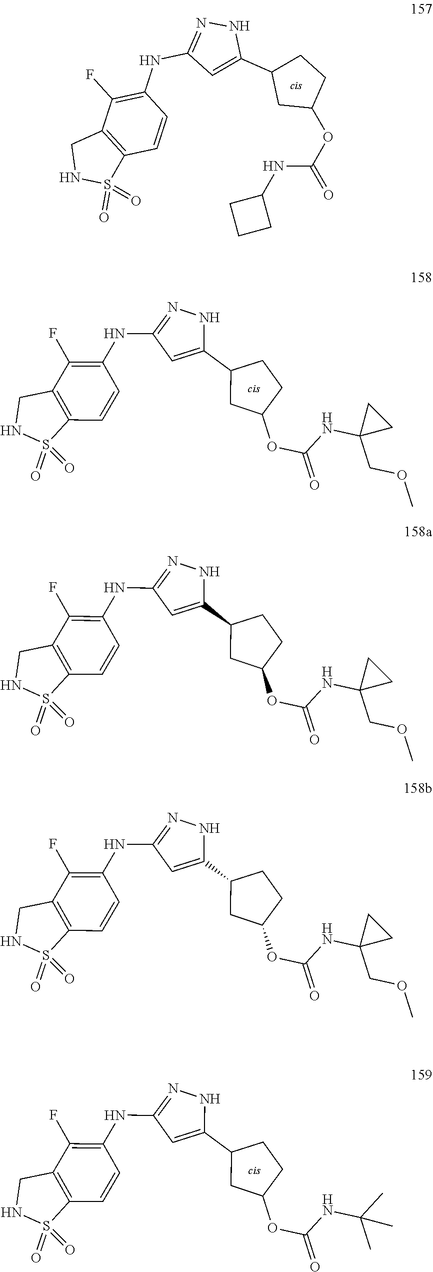 [SMILES: O=C(NC1CCC1)OC1CCC(c2cc(Nc3ccc4c(c3F)CNS4(=O)=O)n[nH]2)C1 ; COCC1(NC(=O)OC2CCC(c3cc(Nc4ccc5c(c4F)CNS5(=O)=O)n[nH]3)C2)CC1 ; COCC1(NC(=O)O[C@@H]2CC[C@H](c3cc(Nc4ccc5c(c4F)CNS5(=O)=O)n[nH]3)C2)CC1 ; COCC1(NC(=O)O[C@H]2CC[C@@H](c3cc(Nc4ccc5c(c4F)CNS5(=O)=O)n[nH]3)C2)CC1 ; CC(C)(C)NC(=O)OC1CCC(c2cc(Nc3ccc4c(c3F)CNS4(=O)=O)n[nH]2)C1]

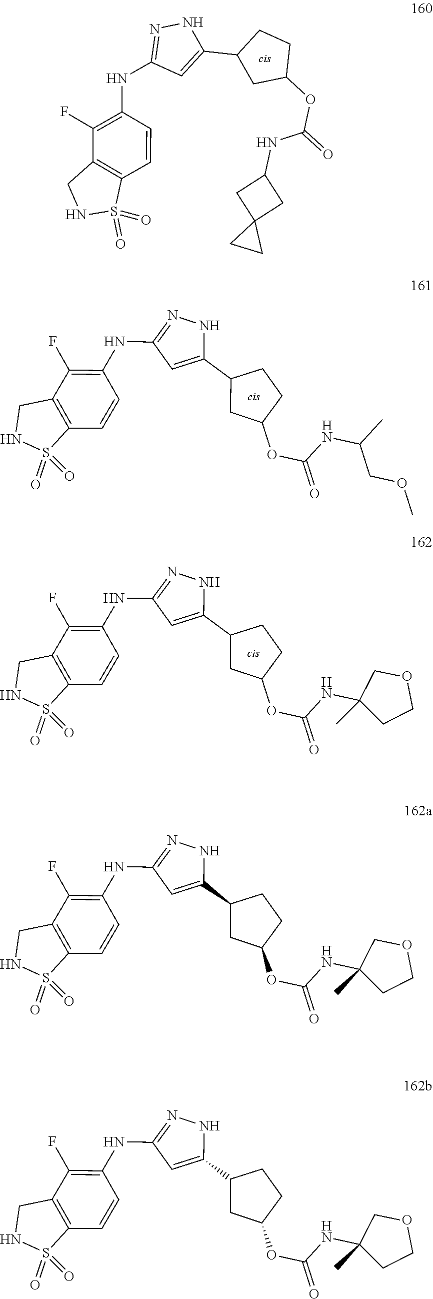

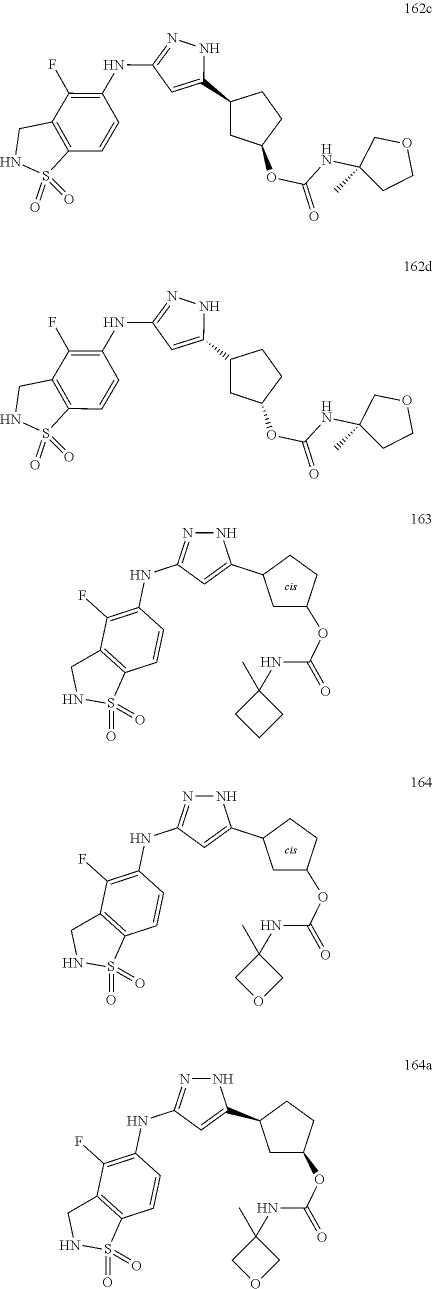

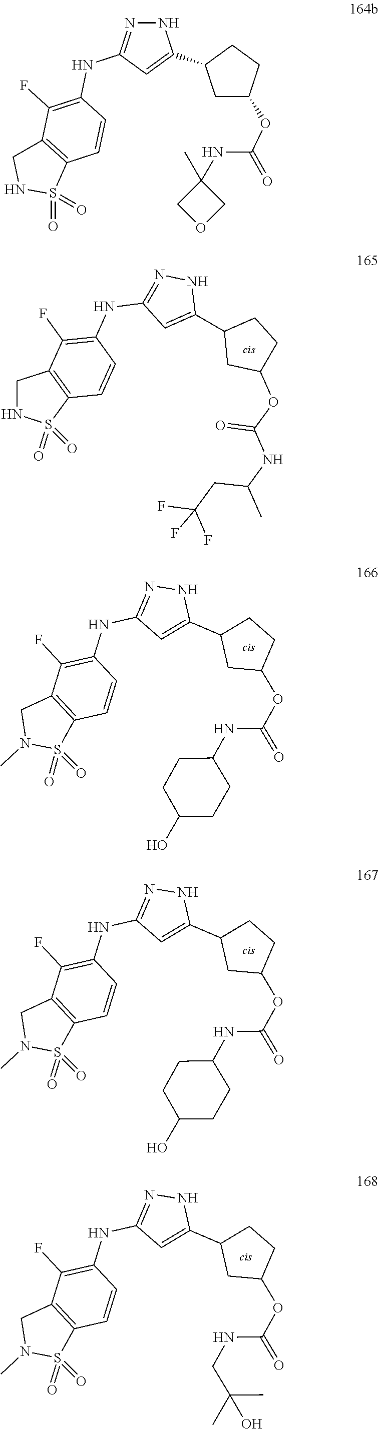

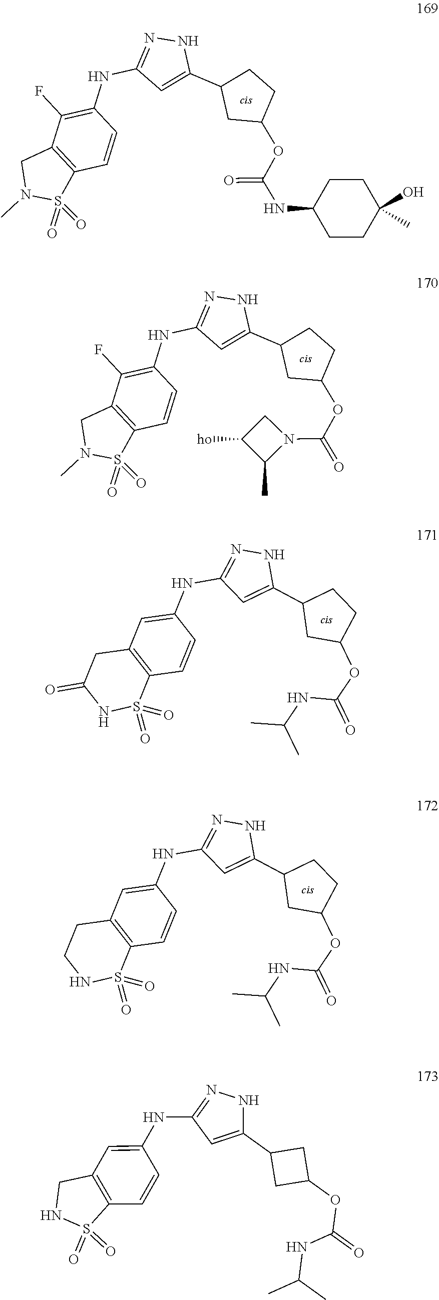

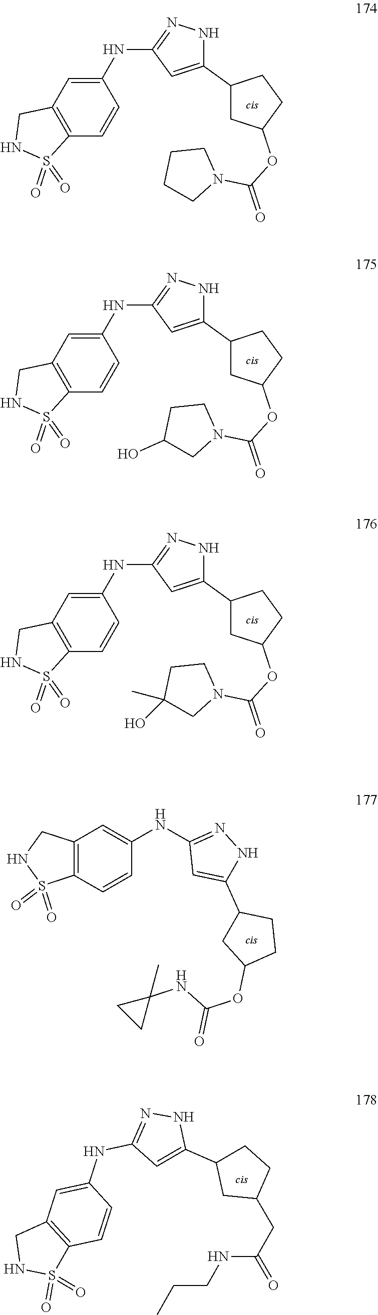 [SMILES: O=C(OC1CCC(c2cc(Nc3ccc4c(c3)CNS4(=O)=O)n[nH]2)C1)N1CCCC1 ; O=C(OC1CCC(c2cc(Nc3ccc4c(c3)CNS4(=O)=O)n[nH]2)C1)N1CCC(O)C1 ; CC1(O)CCN(C(=O)OC2CCC(c3cc(Nc4ccc5c(c4)CNS5(=O)=O)n[nH]3)C2)C1 ; CC1(NC(=O)OC2CCC(c3cc(Nc4ccc5c(c4)CNS5(=O)=O)n[nH]3)C2)CC1 ; CCCNC(=O)CC1CCC(c2cc(Nc3ccc4c(c3)CNS4(=O)=O)n[nH]2)C1]

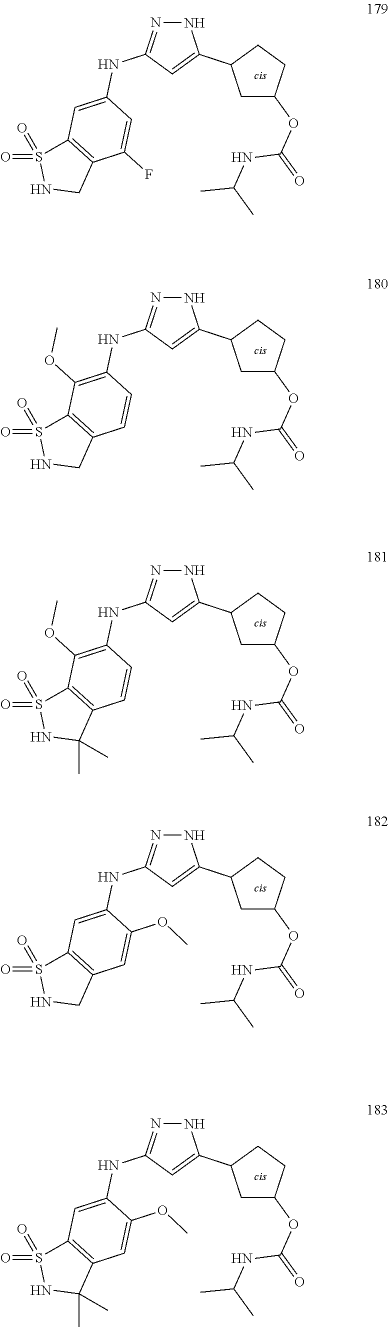 [SMILES: CC(C)NC(=O)OC1CCC(c2cc(Nc3cc(F)c4c(c3)S(=O)(=O)NC4)n[nH]2)C1 ; COc1c(Nc2cc(C3CCC(OC(=O)NC(C)C)C3)[nH]n2)ccc2c1S(=O)(=O)NC2 ; COc1c(Nc2cc(C3CCC(OC(=O)NC(C)C)C3)[nH]n2)ccc2c1S(=O)(=O)NC2(C)C ; COc1cc2c(cc1Nc1cc(C3CCC(OC(=O)NC(C)C)C3)[nH]n1)S(=O)(=O)NC2 ; COc1cc2c(cc1Nc1cc(C3CCC(OC(=O)NC(C)C)C3)[nH]n1)S(=O)(=O)NC2(C)C]

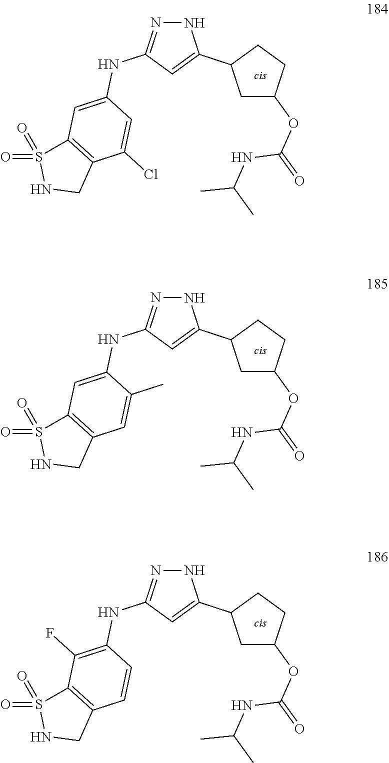 [SMILES: CC(C)NC(=O)OC1CCC(c2cc(Nc3cc(Cl)c4c(c3)S(=O)(=O)NC4)n[nH]2)C1 ; Cc1cc2c(cc1Nc1cc(C3CCC(OC(=O)NC(C)C)C3)[nH]n1)S(=O)(=O)NC2 ; CC(C)NC(=O)OC1CCC(c2cc(Nc3ccc4c(c3F)S(=O)(=O)NC4)n[nH]2)C1]

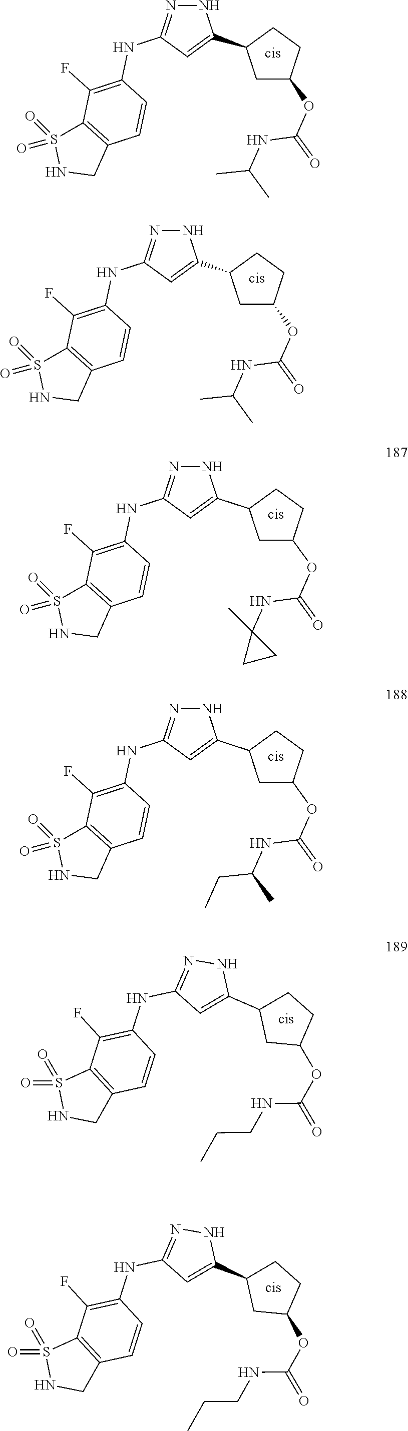 [SMILES: CC(C)NC(=O)O[C@@H]1CC[C@H](c2cc(Nc3ccc4c(c3F)S(=O)(=O)NC4)n[nH]2)C1 ; CC(C)NC(=O)O[C@H]1CC[C@@H](c2cc(Nc3ccc4c(c3F)S(=O)(=O)NC4)n[nH]2)C1 ; CC1(NC(=O)OC2CCC(c3cc(Nc4ccc5c(c4F)S(=O)(=O)NC5)n[nH]3)C2)CC1 ; CC[C@H](C)NC(=O)OC1CCC(c2cc(Nc3ccc4c(c3F)S(=O)(=O)NC4)n[nH]2)C1 ; CCCNC(=O)OC1CCC(c2cc(Nc3ccc4c(c3F)S(=O)(=O)NC4)n[nH]2)C1 ; CCCNC(=O)O[C@@H]1CC[C@H](c2cc(Nc3ccc4c(c3F)S(=O)(=O)NC4)n[nH]2)C1]

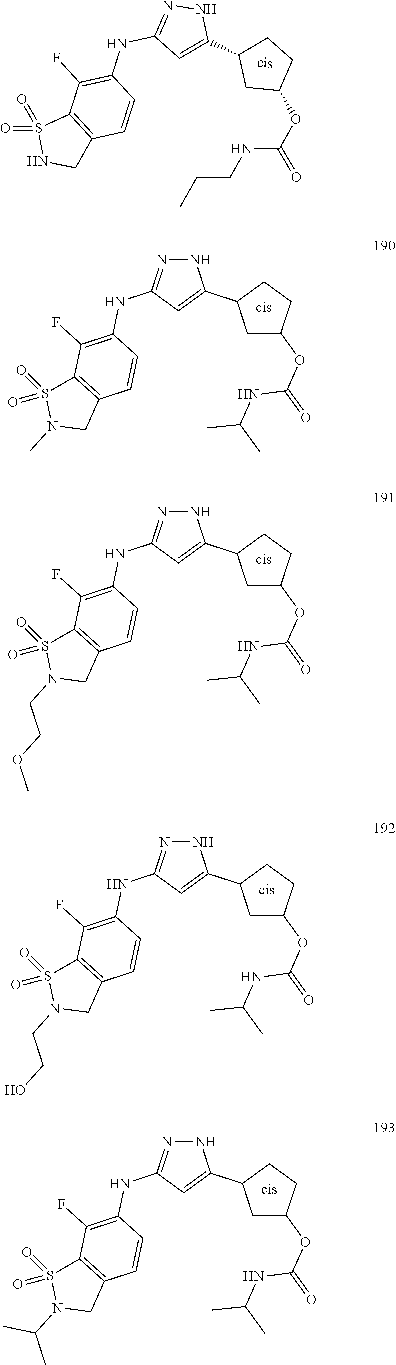 [SMILES: CCCNC(=O)O[C@H]1CC[C@@H](c2cc(Nc3ccc4c(c3F)S(=O)(=O)NC4)n[nH]2)C1 ; CC(C)NC(=O)OC1CCC(c2cc(Nc3ccc4c(c3F)S(=O)(=O)N(C)C4)n[nH]2)C1 ; COCCN1Cc2ccc(Nc3cc(C4CCC(OC(=O)NC(C)C)C4)[nH]n3)c(F)c2S1(=O)=O ; CC(C)NC(=O)OC1CCC(c2cc(Nc3ccc4c(c3F)S(=O)(=O)N(CCO)C4)n[nH]2)C1 ; CC(C)NC(=O)OC1CCC(c2cc(Nc3ccc4c(c3F)S(=O)(=O)N(C(C)C)C4)n[nH]2)C1]

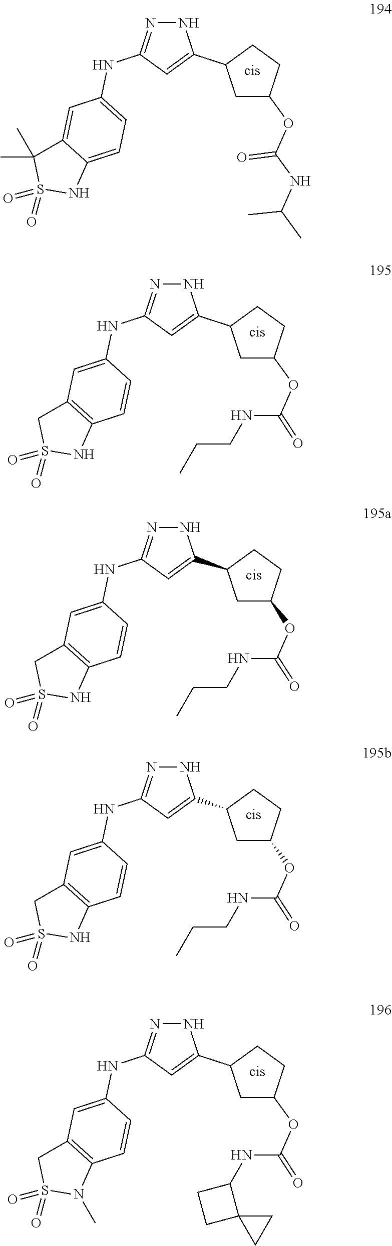 [SMILES: CC(C)NC(=O)OC1CCC(c2cc(Nc3ccc4c(c3)C(C)(C)S(=O)(=O)N4)n[nH]2)C1 ; CCCNC(=O)OC1CCC(c2cc(Nc3ccc4c(c3)CS(=O)(=O)N4)n[nH]2)C1 ; CCCNC(=O)O[C@@H]1CC[C@H](c2cc(Nc3ccc4c(c3)CS(=O)(=O)N4)n[nH]2)C1 ; CCCNC(=O)O[C@H]1CC[C@@H](c2cc(Nc3ccc4c(c3)CS(=O)(=O)N4)n[nH]2)C1 ; CN1c2ccc(Nc3cc(C4CCC(OC(=O)NC5CCC56CC6)C4)[nH]n3)cc2CS1(=O)=O]

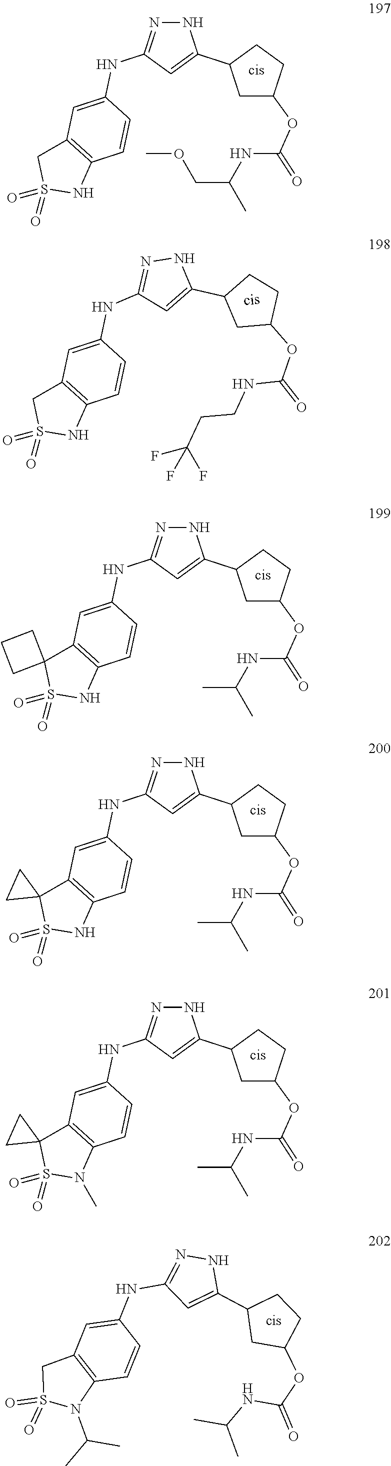 [SMILES: COCC(C)NC(=O)OC1CCC(c2cc(Nc3ccc4c(c3)CS(=O)(=O)N4)n[nH]2)C1 ; O=C(NCCC(F)(F)F)OC1CCC(c2cc(Nc3ccc4c(c3)CS(=O)(=O)N4)n[nH]2)C1 ; CC(C)NC(=O)OC1CCC(c2cc(Nc3ccc4c(c3)C3(CCC3)S(=O)(=O)N4)n[nH]2)C1 ; CC(C)NC(=O)OC1CCC(c2cc(Nc3ccc4c(c3)C3(CC3)S(=O)(=O)N4)n[nH]2)C1 ; CC(C)NC(=O)OC1CCC(c2cc(Nc3ccc4c(c3)C3(CC3)S(=O)(=O)N4C)n[nH]2)C1 ; CC(C)NC(=O)OC1CCC(c2cc(Nc3ccc4c(c3)CS(=O)(=O)N4C(C)C)n[nH]2)C1]

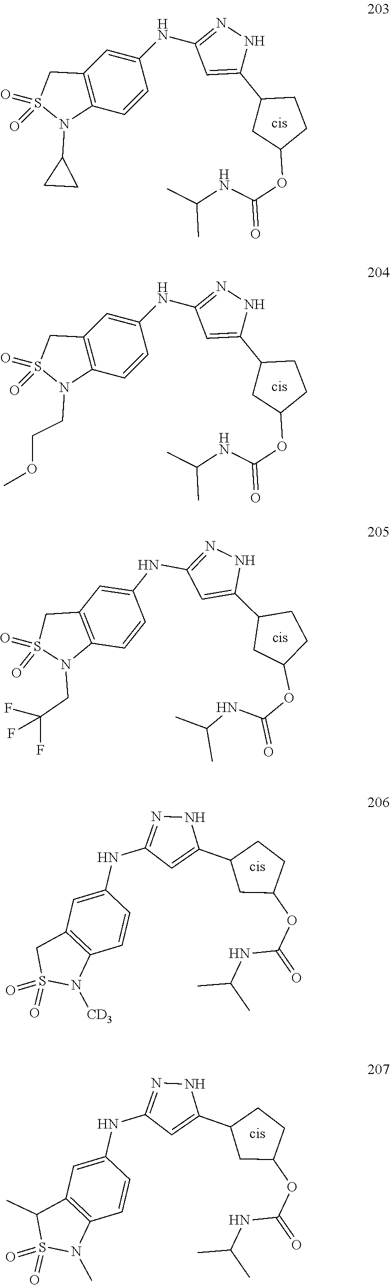

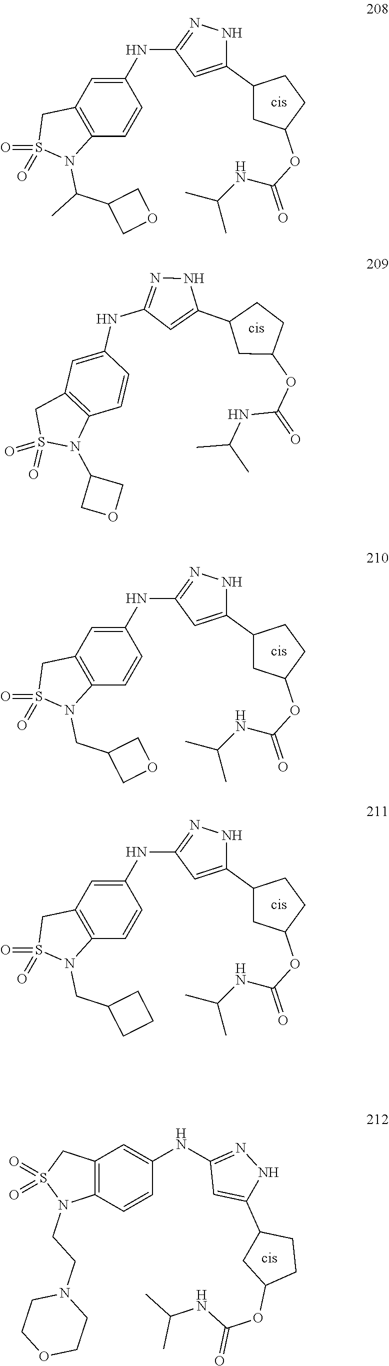 [SMILES: CC(C)NC(=O)OC1CCC(c2cc(Nc3ccc4c(c3)CS(=O)(=O)N4C(C)C3COC3)n[nH]2)C1 ; CC(C)NC(=O)OC1CCC(c2cc(Nc3ccc4c(c3)CS(=O)(=O)N4C3COC3)n[nH]2)C1 ; CC(C)NC(=O)OC1CCC(c2cc(Nc3ccc4c(c3)CS(=O)(=O)N4CC3COC3)n[nH]2)C1 ; CC(C)NC(=O)OC1CCC(c2cc(Nc3ccc4c(c3)CS(=O)(=O)N4CC3CCC3)n[nH]2)C1 ; CC(C)NC(=O)OC1CCC(c2cc(Nc3ccc4c(c3)CS(=O)(=O)N4CCN3CCOCC3)n[nH]2)C1]

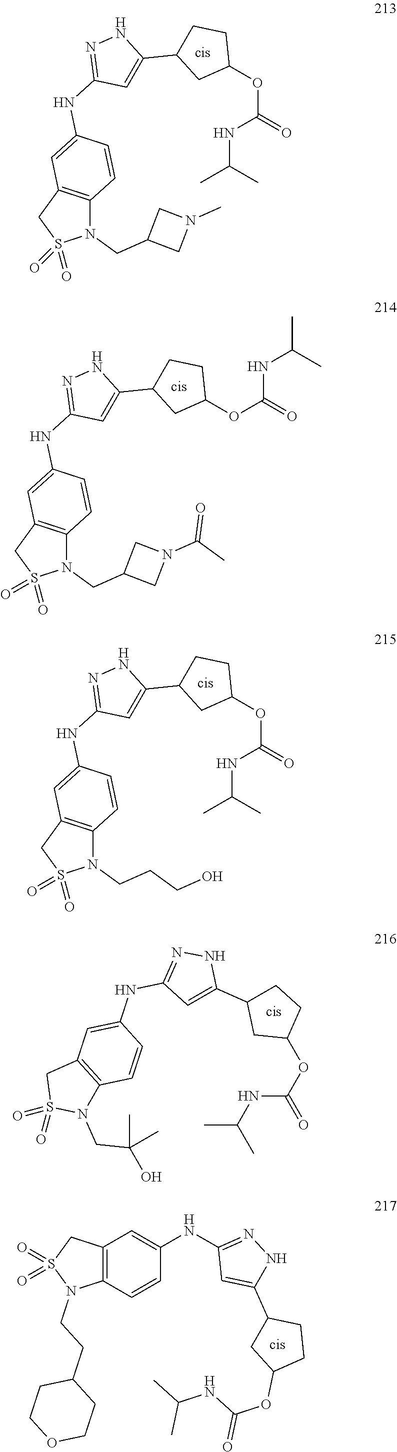 [SMILES: CC(C)NC(=O)OC1CCC(c2cc(Nc3ccc4c(c3)CS(=O)(=O)N4CC3CN(C)C3)n[nH]2)C1 ; CC(=O)N1CC(CN2c3ccc(Nc4cc(C5CCC(OC(=O)NC(C)C)C5)[nH]n4)cc3CS2(=O)=O)C1 ; CC(C)NC(=O)OC1CCC(c2cc(Nc3ccc4c(c3)CS(=O)(=O)N4CCCO)n[nH]2)C1 ; CC(C)NC(=O)OC1CCC(c2cc(Nc3ccc4c(c3)CS(=O)(=O)N4CC(C)(C)O)n[nH]2)C1 ; CC(C)NC(=O)OC1CCC(c2cc(Nc3ccc4c(c3)CS(=O)(=O)N4CCC3CCOCC3)n[nH]2)C1]

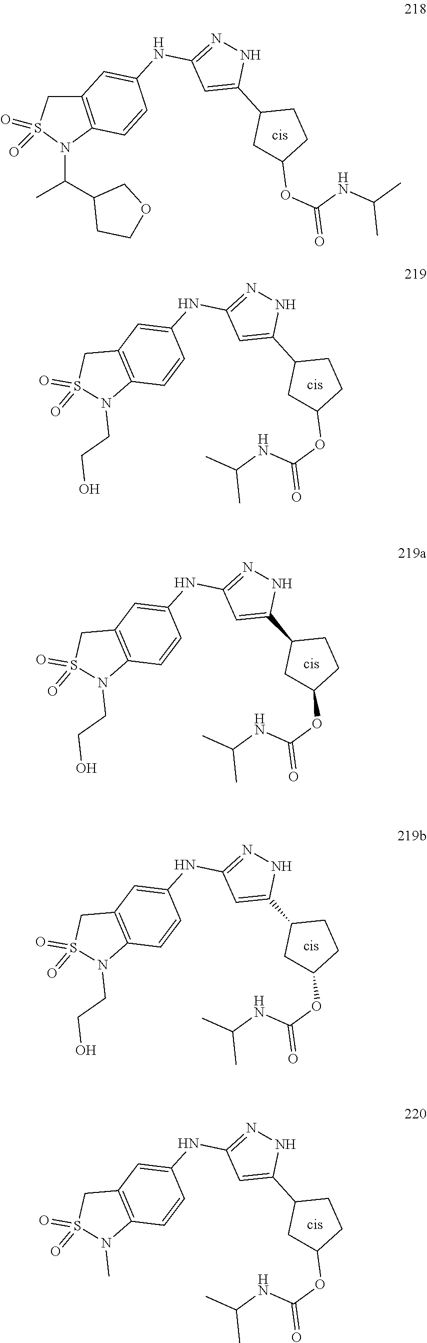 [SMILES: CC(C)NC(=O)OC1CCC(c2cc(Nc3ccc4c(c3)CS(=O)(=O)N4C(C)C3CCOC3)n[nH]2)C1 ; CC(C)NC(=O)OC1CCC(c2cc(Nc3ccc4c(c3)CS(=O)(=O)N4CCO)n[nH]2)C1 ; CC(C)NC(=O)O[C@@H]1CC[C@H](c2cc(Nc3ccc4c(c3)CS(=O)(=O)N4CCO)n[nH]2)C1 ; CC(C)NC(=O)O[C@H]1CC[C@@H](c2cc(Nc3ccc4c(c3)CS(=O)(=O)N4CCO)n[nH]2)C1 ; CC(C)NC(=O)OC1CCC(c2cc(Nc3ccc4c(c3)CS(=O)(=O)N4C)n[nH]2)C1]

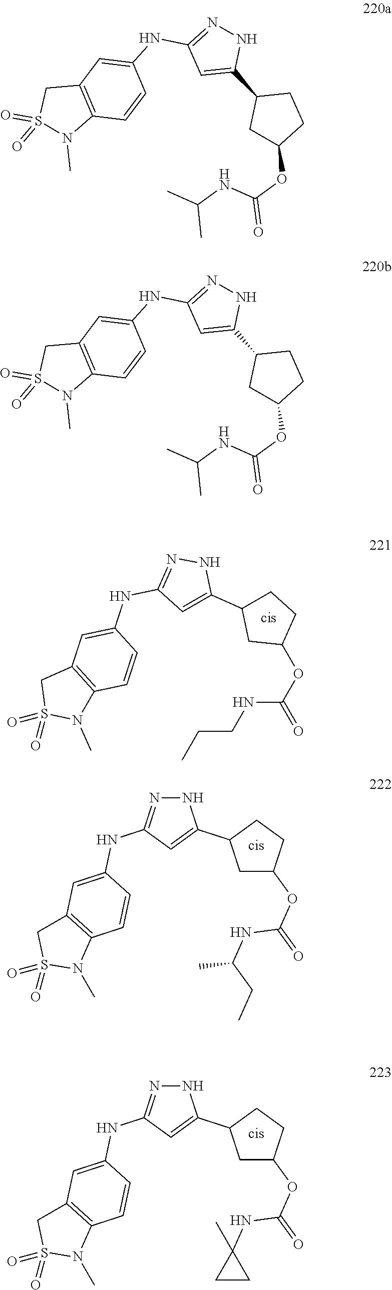

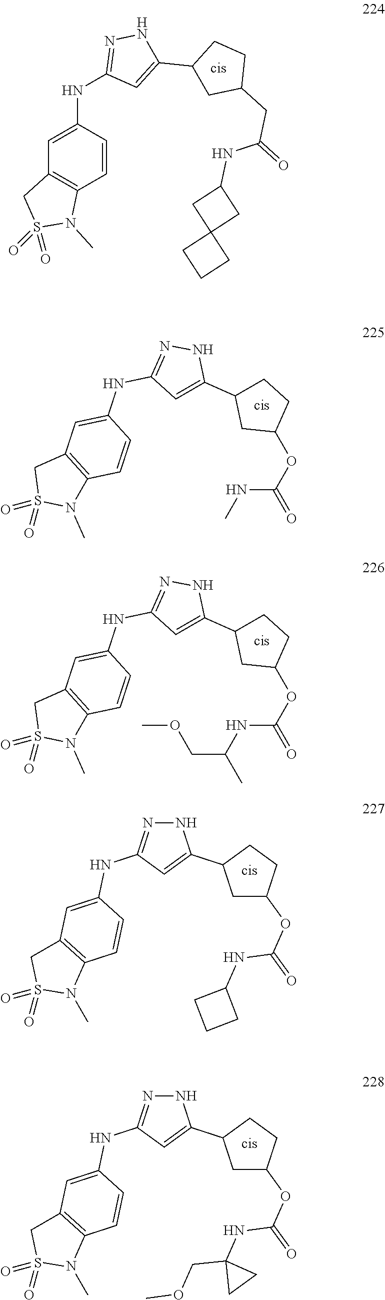 [SMILES: CN1c2ccc(Nc3cc(C4CCC(CC(=O)NC5CC6(CCC6)C5)C4)[nH]n3)cc2CS1(=O)=O ; CNC(=O)OC1CCC(c2cc(Nc3ccc4c(c3)CS(=O)(=O)N4C)n[nH]2)C1 ; COCC(C)NC(=O)OC1CCC(c2cc(Nc3ccc4c(c3)CS(=O)(=O)N4C)n[nH]2)C1 ; CN1c2ccc(Nc3cc(C4CCC(OC(=O)NC5CCC5)C4)[nH]n3)cc2CS1(=O)=O ; COCC1(NC(=O)OC2CCC(c3cc(Nc4ccc5c(c4)CS(=O)(=O)N5C)n[nH]3)C2)CC1]

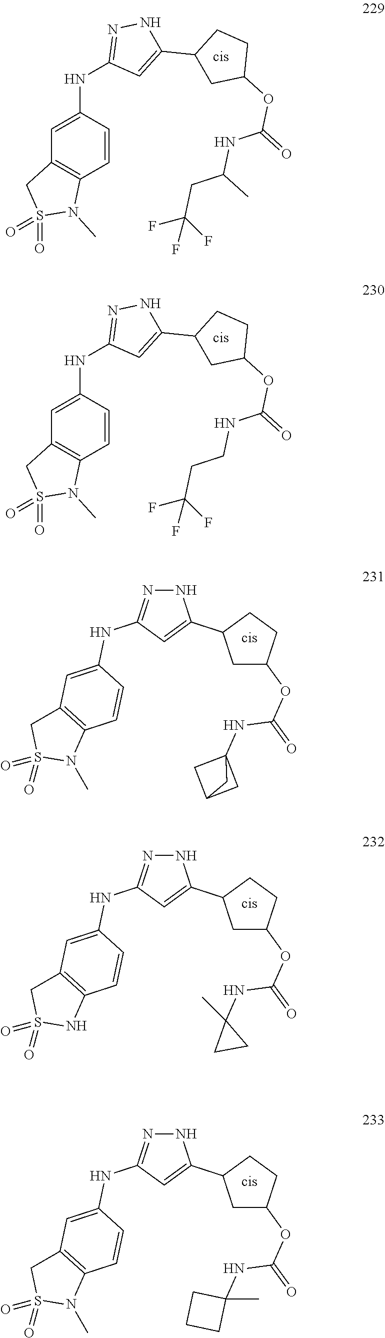 [SMILES: CC(CC(F)(F)F)NC(=O)OC1CCC(c2cc(Nc3ccc4c(c3)CS(=O)(=O)N4C)n[nH]2)C1 ; CN1c2ccc(Nc3cc(C4CCC(OC(=O)NCCC(F)(F)F)C4)[nH]n3)cc2CS1(=O)=O ; CN1c2ccc(Nc3cc(C4CCC(OC(=O)NC56CC(C5)C6)C4)[nH]n3)cc2CS1(=O)=O ; CC1(NC(=O)OC2CCC(c3cc(Nc4ccc5c(c4)CS(=O)(=O)N5)n[nH]3)C2)CC1 ; CN1c2ccc(Nc3cc(C4CCC(OC(=O)NC5(C)CCC5)C4)[nH]n3)cc2CS1(=O)=O]

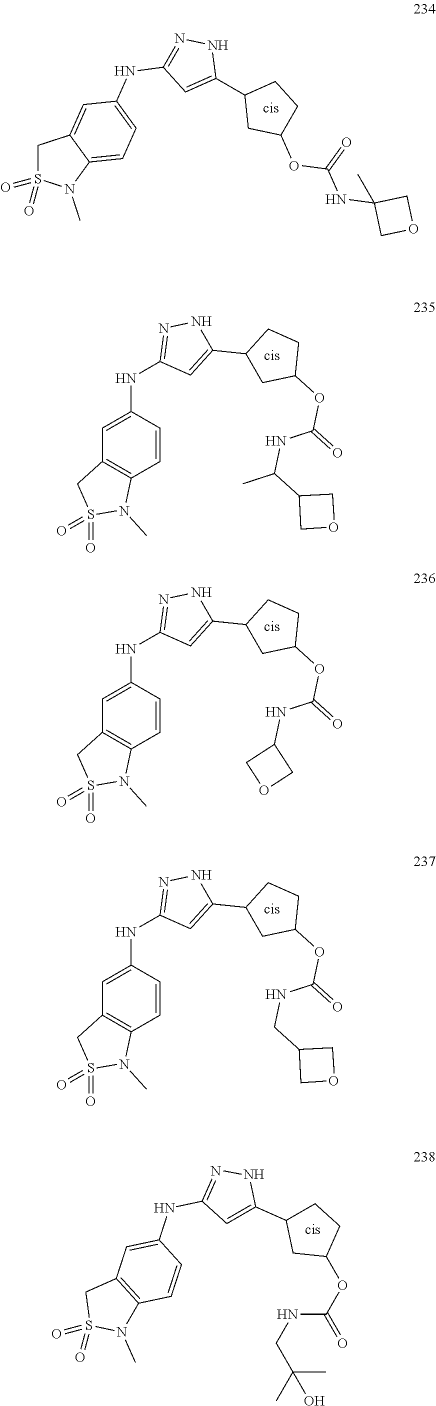 [SMILES: CN1c2ccc(Nc3cc(C4CCC(OC(=O)NC5(C)COC5)C4)[nH]n3)cc2CS1(=O)=O ; CC(NC(=O)OC1CCC(c2cc(Nc3ccc4c(c3)CS(=O)(=O)N4C)n[nH]2)C1)C1COC1 ; CN1c2ccc(Nc3cc(C4CCC(OC(=O)NC5COC5)C4)[nH]n3)cc2CS1(=O)=O ; CN1c2ccc(Nc3cc(C4CCC(OC(=O)NCC5COC5)C4)[nH]n3)cc2CS1(=O)=O ; CN1c2ccc(Nc3cc(C4CCC(OC(=O)NCC(C)(C)O)C4)[nH]n3)cc2CS1(=O)=O]

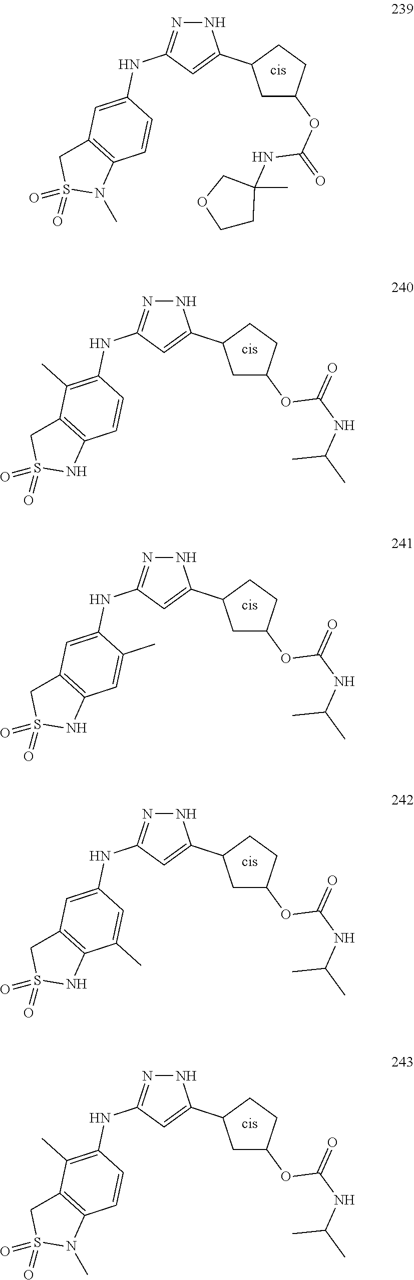 [SMILES: CN1c2ccc(Nc3cc(C4CCC(OC(=O)NC5(C)CCOC5)C4)[nH]n3)cc2CS1(=O)=O ; Cc1c(Nc2cc(C3CCC(OC(=O)NC(C)C)C3)[nH]n2)ccc2c1CS(=O)(=O)N2 ; Cc1cc2c(cc1Nc1cc(C3CCC(OC(=O)NC(C)C)C3)[nH]n1)CS(=O)(=O)N2 ; Cc1cc(Nc2cc(C3CCC(OC(=O)NC(C)C)C3)[nH]n2)cc2c1NS(=O)(=O)C2 ; Cc1c(Nc2cc(C3CCC(OC(=O)NC(C)C)C3)[nH]n2)ccc2c1CS(=O)(=O)N2C]

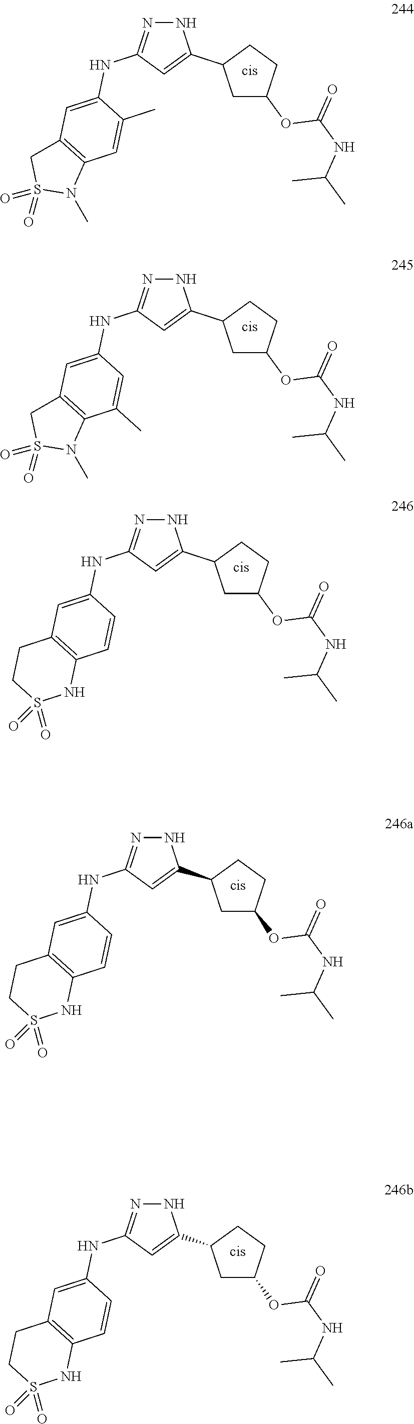 [SMILES: Cc1cc2c(cc1Nc1cc(C3CCC(OC(=O)NC(C)C)C3)[nH]n1)CS(=O)(=O)N2C ; Cc1cc(Nc2cc(C3CCC(OC(=O)NC(C)C)C3)[nH]n2)cc2c1N(C)S(=O)(=O)C2 ; CC(C)NC(=O)OC1CCC(c2cc(Nc3ccc4c(c3)CCS(=O)(=O)N4)n[nH]2)C1 ; CC(C)NC(=O)O[C@@H]1CC[C@H](c2cc(Nc3ccc4c(c3)CCS(=O)(=O)N4)n[nH]2)C1 ; CC(C)NC(=O)O[C@H]1CC[C@@H](c2cc(Nc3ccc4c(c3)CCS(=O)(=O)N4)n[nH]2)C1]

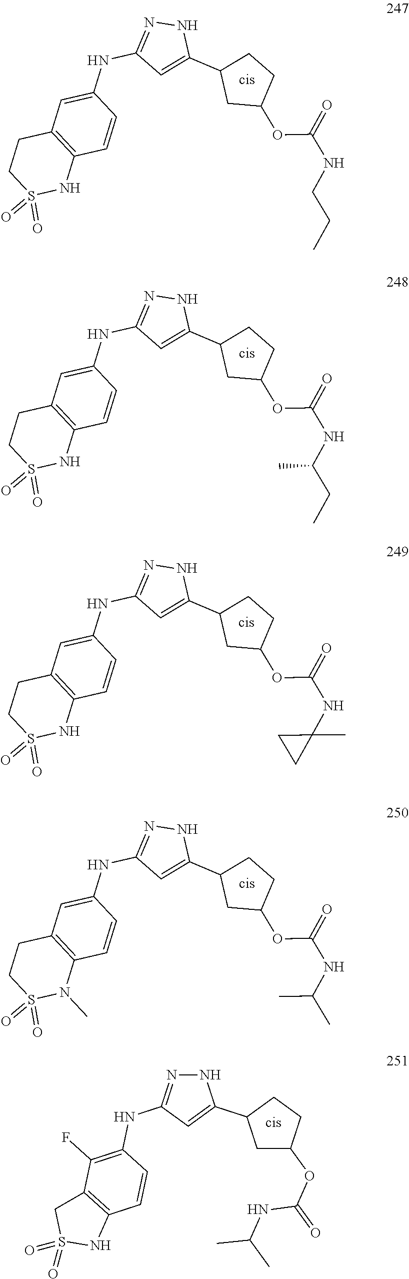 [SMILES: CCCNC(=O)OC1CCC(c2cc(Nc3ccc4c(c3)CCS(=O)(=O)N4)n[nH]2)C1 ; CC[C@H](C)NC(=O)OC1CCC(c2cc(Nc3ccc4c(c3)CCS(=O)(=O)N4)n[nH]2)C1 ; CC1(NC(=O)OC2CCC(c3cc(Nc4ccc5c(c4)CCS(=O)(=O)N5)n[nH]3)C2)CC1 ; CC(C)NC(=O)OC1CCC(c2cc(Nc3ccc4c(c3)CCS(=O)(=O)N4C)n[nH]2)C1 ; CC(C)NC(=O)OC1CCC(c2cc(Nc3ccc4c(c3F)CS(=O)(=O)N4)n[nH]2)C1]

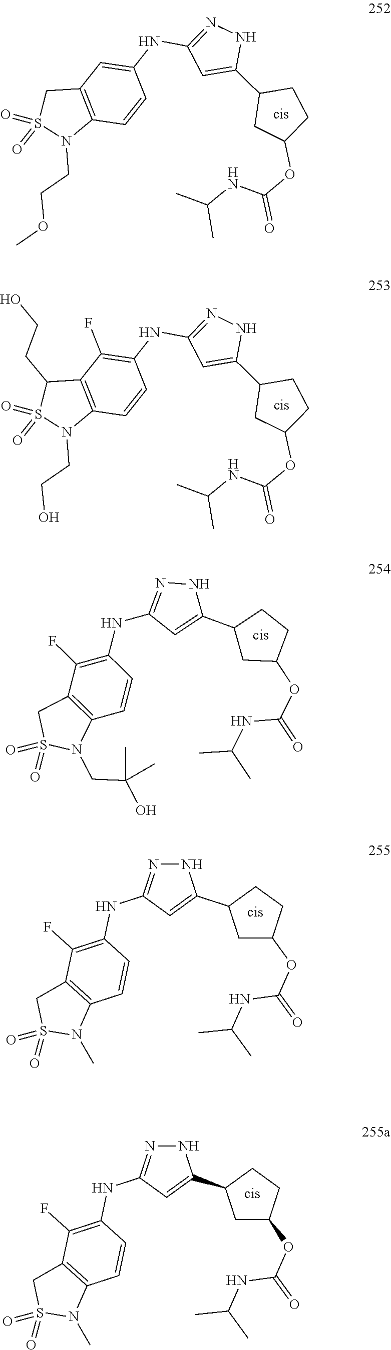 [SMILES: COCCN1c2ccc(Nc3cc(C4CCC(OC(=O)NC(C)C)C4)[nH]n3)cc2CS1(=O)=O ; CC(C)NC(=O)OC1CCC(c2cc(Nc3ccc4c(c3F)C(CCO)S(=O)(=O)N4CCO)n[nH]2)C1 ; CC(C)NC(=O)OC1CCC(c2cc(Nc3ccc4c(c3F)CS(=O)(=O)N4CC(C)(C)O)n[nH]2)C1 ; CC(C)NC(=O)OC1CCC(c2cc(Nc3ccc4c(c3F)CS(=O)(=O)N4C)n[nH]2)C1 ; CC(C)NC(=O)O[C@@H]1CC[C@H](c2cc(Nc3ccc4c(c3F)CS(=O)(=O)N4C)n[nH]2)C1]

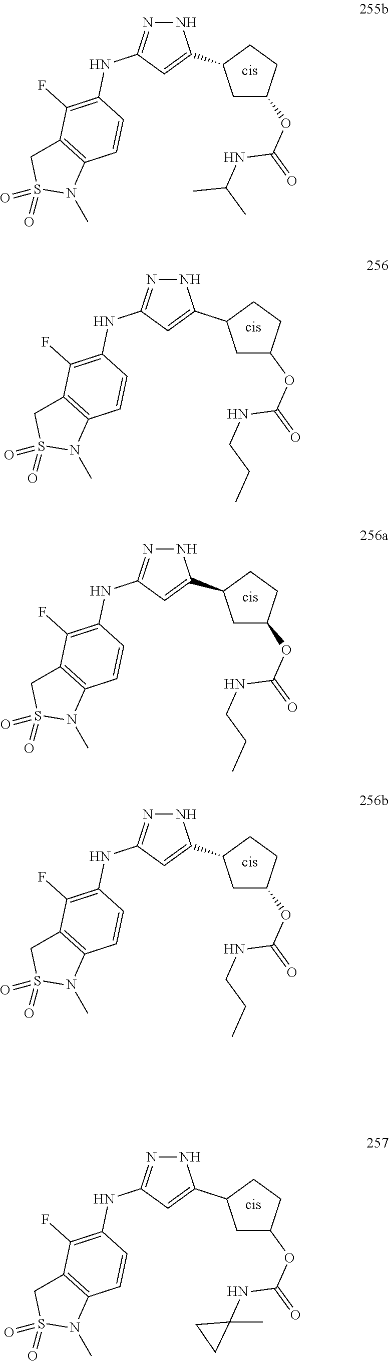 [SMILES: CC(C)NC(=O)O[C@H]1CC[C@@H](c2cc(Nc3ccc4c(c3F)CS(=O)(=O)N4C)n[nH]2)C1 ; CCCNC(=O)OC1CCC(c2cc(Nc3ccc4c(c3F)CS(=O)(=O)N4C)n[nH]2)C1 ; CCCNC(=O)O[C@@H]1CC[C@H](c2cc(Nc3ccc4c(c3F)CS(=O)(=O)N4C)n[nH]2)C1 ; CCCNC(=O)O[C@H]1CC[C@@H](c2cc(Nc3ccc4c(c3F)CS(=O)(=O)N4C)n[nH]2)C1 ; CN1c2ccc(Nc3cc(C4CCC(OC(=O)NC5(C)CC5)C4)[nH]n3)c(F)c2CS1(=O)=O]

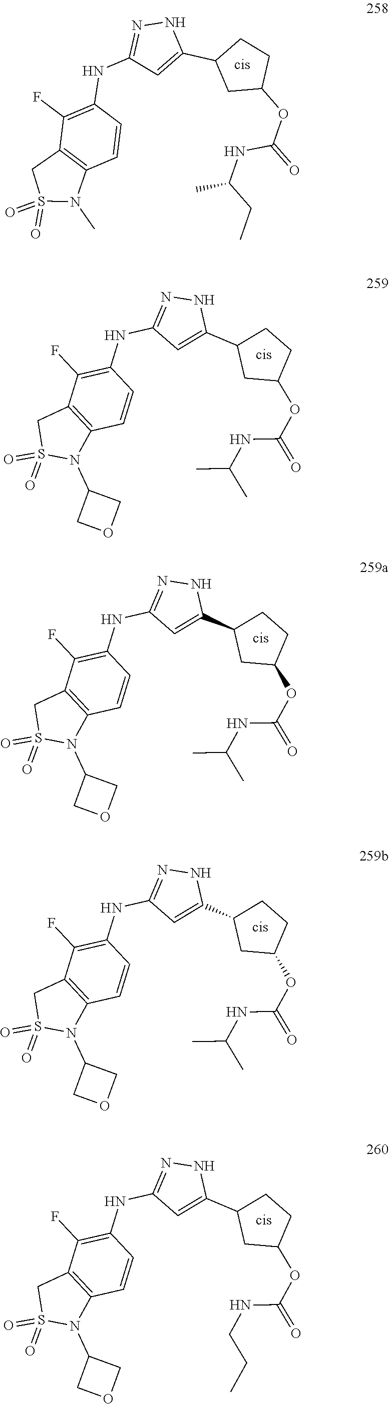 [SMILES: CC[C@H](C)NC(=O)OC1CCC(c2cc(Nc3ccc4c(c3F)CS(=O)(=O)N4C)n[nH]2)C1 ; CC(C)NC(=O)OC1CCC(c2cc(Nc3ccc4c(c3F)CS(=O)(=O)N4C3COC3)n[nH]2)C1 ; CC(C)NC(=O)O[C@@H]1CC[C@H](c2cc(Nc3ccc4c(c3F)CS(=O)(=O)N4C3COC3)n[nH]2)C1 ; CC(C)NC(=O)O[C@H]1CC[C@@H](c2cc(Nc3ccc4c(c3F)CS(=O)(=O)N4C3COC3)n[nH]2)C1 ; CCCNC(=O)OC1CCC(c2cc(Nc3ccc4c(c3F)CS(=O)(=O)N4C3COC3)n[nH]2)C1]

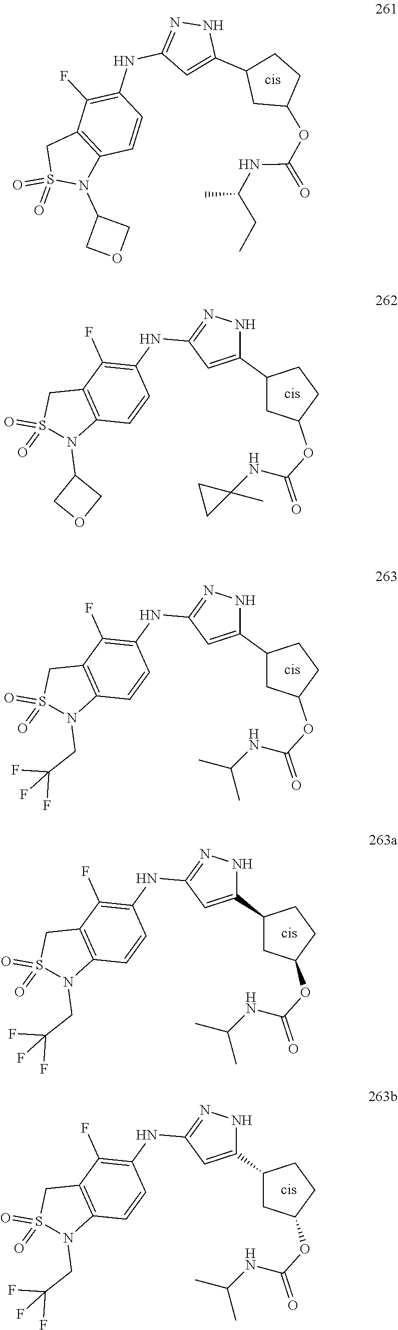 [SMILES: CC[C@H](C)NC(=O)OC1CCC(c2cc(Nc3ccc4c(c3F)CS(=O)(=O)N4C3COC3)n[nH]2)C1 ; CC1(NC(=O)OC2CCC(c3cc(Nc4ccc5c(c4F)CS(=O)(=O)N5C4COC4)n[nH]3)C2)CC1 ; CC(C)NC(=O)OC1CCC(c2cc(Nc3ccc4c(c3F)CS(=O)(=O)N4CC(F)(F)F)n[nH]2)C1 ; CC(C)NC(=O)O[C@@H]1CC[C@H](c2cc(Nc3ccc4c(c3F)CS(=O)(=O)N4CC(F)(F)F)n[nH]2)C1 ; CC(C)NC(=O)O[C@H]1CC[C@@H](c2cc(Nc3ccc4c(c3F)CS(=O)(=O)N4CC(F)(F)F)n[nH]2)C1]

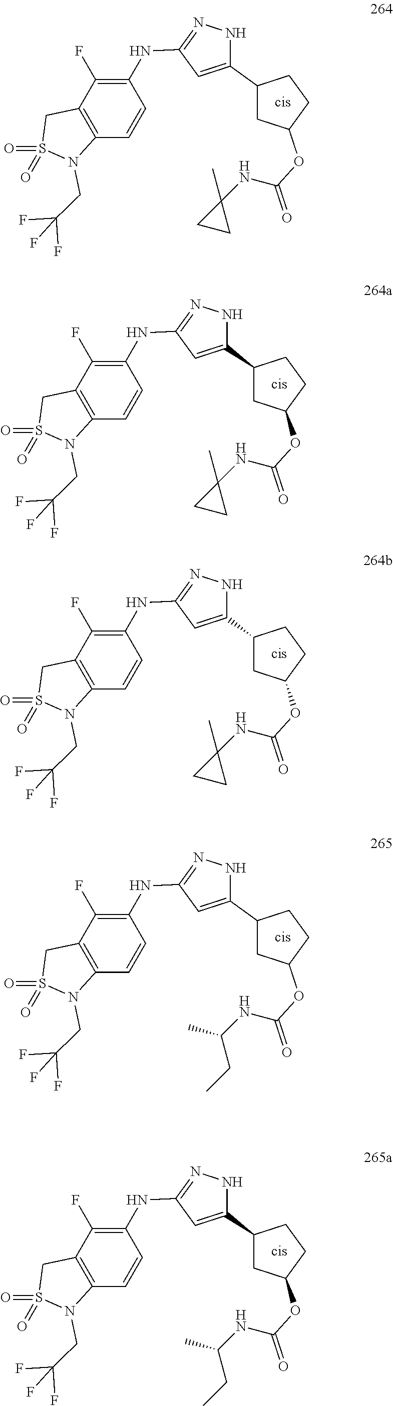 [SMILES: CC1(NC(=O)OC2CCC(c3cc(Nc4ccc5c(c4F)CS(=O)(=O)N5CC(F)(F)F)n[nH]3)C2)CC1 ; CC1(NC(=O)O[C@@H]2CC[C@H](c3cc(Nc4ccc5c(c4F)CS(=O)(=O)N5CC(F)(F)F)n[nH]3)C2)CC1 ; CC1(NC(=O)O[C@H]2CC[C@@H](c3cc(Nc4ccc5c(c4F)CS(=O)(=O)N5CC(F)(F)F)n[nH]3)C2)CC1 ; CC[C@H](C)NC(=O)OC1CCC(c2cc(Nc3ccc4c(c3F)CS(=O)(=O)N4CC(F)(F)F)n[nH]2)C1 ; CC[C@H](C)NC(=O)O[C@@H]1CC[C@H](c2cc(Nc3ccc4c(c3F)CS(=O)(=O)N4CC(F)(F)F)n[nH]2)C1]

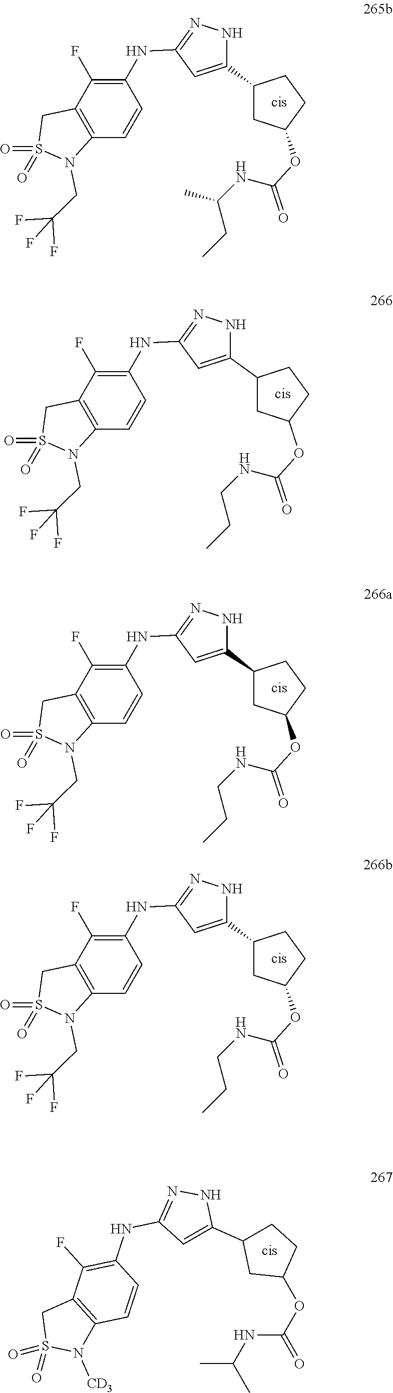

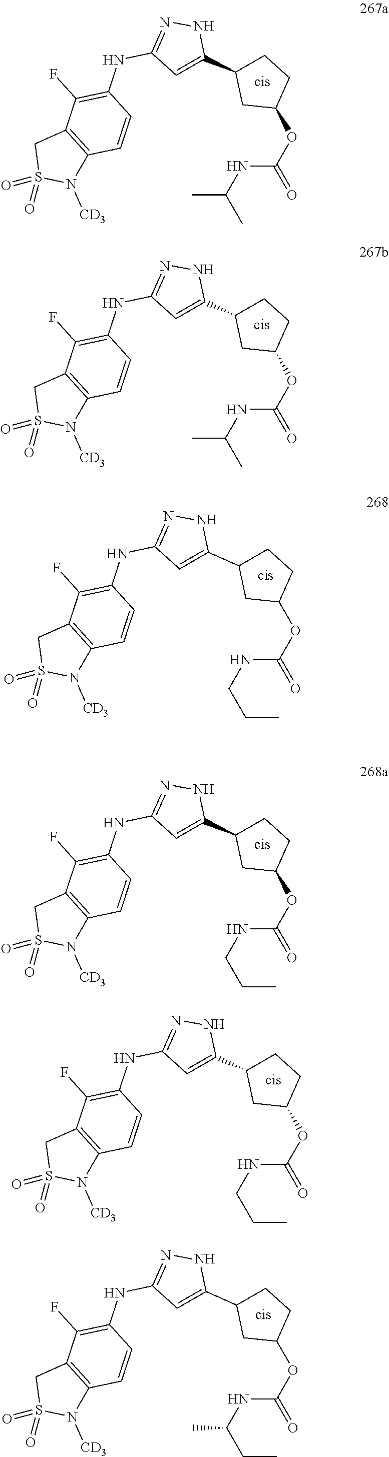

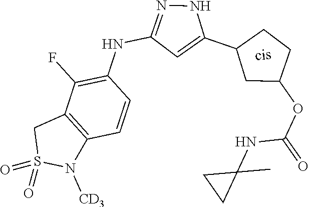 [SMILES: [2H]C([2H])([2H])N1c2ccc(Nc3cc(C4CCC(OC(=O)NC5(C)CC5)C4)[nH]n3)c(F)c2CS1(=O)=O]

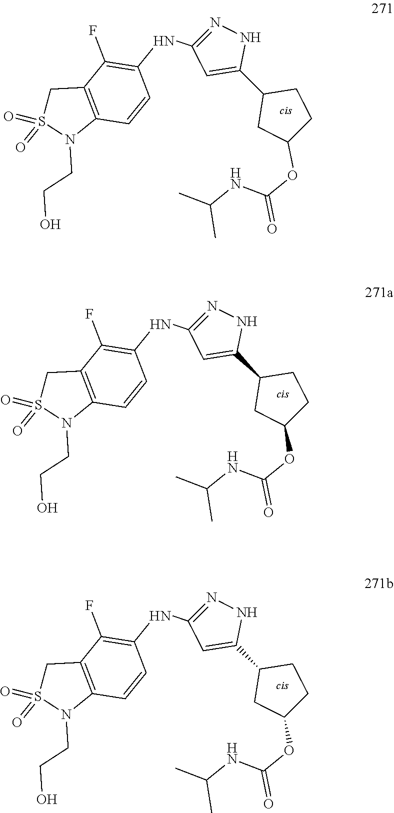 [SMILES: CC(C)NC(=O)OC1CCC(c2cc(Nc3ccc4c(c3F)CS(=O)(=O)N4CCO)n[nH]2)C1 ; CC(C)NC(=O)O[C@@H]1CC[C@H](c2cc(Nc3ccc4c(c3F)CS(=O)(=O)N4CCO)n[nH]2)C1 ; CC(C)NC(=O)O[C@H]1CC[C@@H](c2cc(Nc3ccc4c(c3F)CS(=O)(=O)N4CCO)n[nH]2)C1]

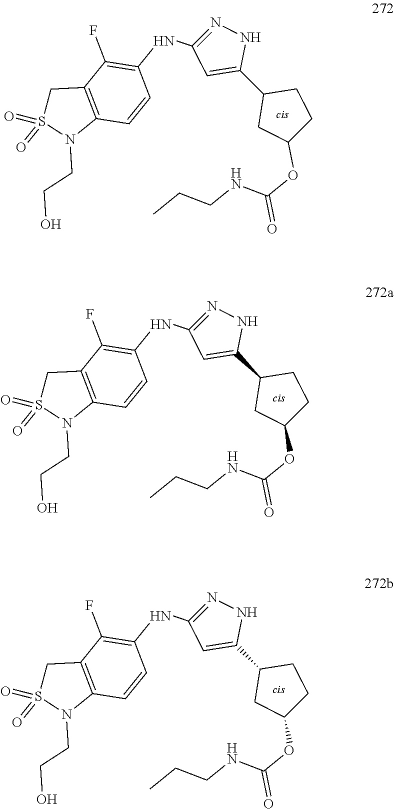 [SMILES: CCCNC(=O)OC1CCC(c2cc(Nc3ccc4c(c3F)CS(=O)(=O)N4CCO)n[nH]2)C1 ; CCCNC(=O)O[C@@H]1CC[C@H](c2cc(Nc3ccc4c(c3F)CS(=O)(=O)N4CCO)n[nH]2)C1 ; CCCNC(=O)O[C@H]1CC[C@@H](c2cc(Nc3ccc4c(c3F)CS(=O)(=O)N4CCO)n[nH]2)C1]

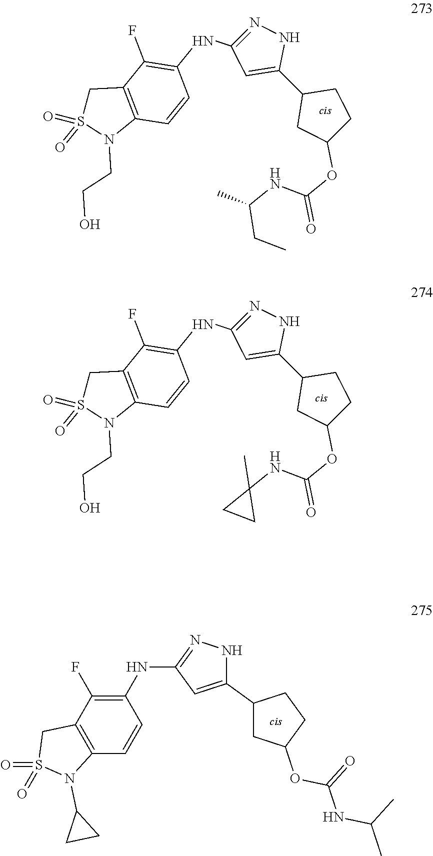 [SMILES: CC[C@H](C)NC(=O)OC1CCC(c2cc(Nc3ccc4c(c3F)CS(=O)(=O)N4CCO)n[nH]2)C1 ; CC1(NC(=O)OC2CCC(c3cc(Nc4ccc5c(c4F)CS(=O)(=O)N5CCO)n[nH]3)C2)CC1 ; CC(C)NC(=O)OC1CCC(c2cc(Nc3ccc4c(c3F)CS(=O)(=O)N4C3CC3)n[nH]2)C1]

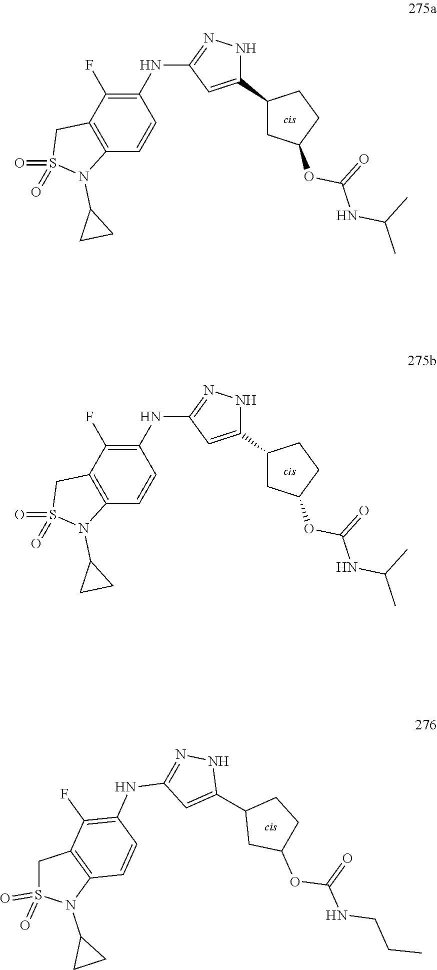 [SMILES: CC(C)NC(=O)O[C@@H]1CC[C@H](c2cc(Nc3ccc4c(c3F)CS(=O)(=O)N4C3CC3)n[nH]2)C1 ; CC(C)NC(=O)O[C@H]1CC[C@@H](c2cc(Nc3ccc4c(c3F)CS(=O)(=O)N4C3CC3)n[nH]2)C1 ; CCCNC(=O)OC1CCC(c2cc(Nc3ccc4c(c3F)CS(=O)(=O)N4C3CC3)n[nH]2)C1]

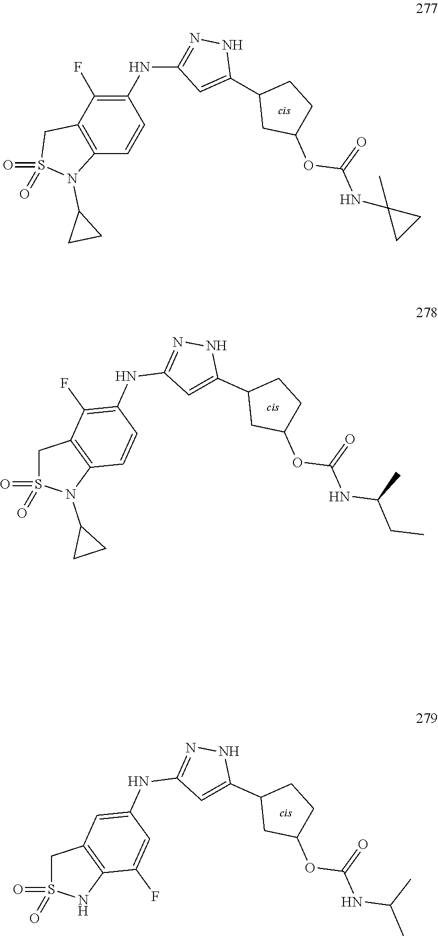 [SMILES: CC1(NC(=O)OC2CCC(c3cc(Nc4ccc5c(c4F)CS(=O)(=O)N5C4CC4)n[nH]3)C2)CC1 ; CC[C@H](C)NC(=O)OC1CCC(c2cc(Nc3ccc4c(c3F)CS(=O)(=O)N4C3CC3)n[nH]2)C1 ; CC(C)NC(=O)OC1CCC(c2cc(Nc3cc(F)c4c(c3)CS(=O)(=O)N4)n[nH]2)C1]

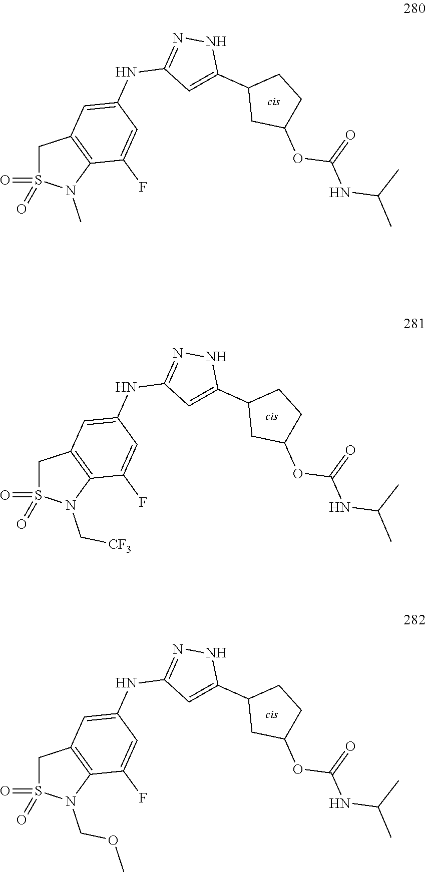 [SMILES: CC(C)NC(=O)OC1CCC(c2cc(Nc3cc(F)c4c(c3)CS(=O)(=O)N4C)n[nH]2)C1 ; CC(C)NC(=O)OC1CCC(c2cc(Nc3cc(F)c4c(c3)CS(=O)(=O)N4CC(F)(F)F)n[nH]2)C1 ; COCN1c2c(F)cc(Nc3cc(C4CCC(OC(=O)NC(C)C)C4)[nH]n3)cc2CS1(=O)=O]

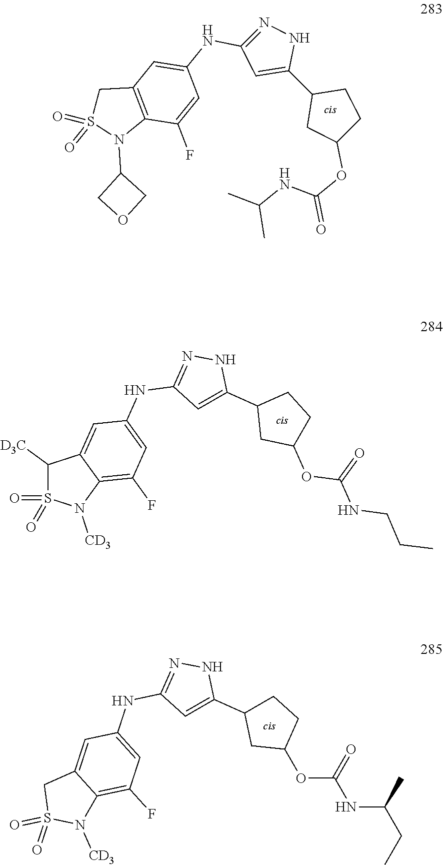 [SMILES: CC(C)NC(=O)OC1CCC(c2cc(Nc3cc(F)c4c(c3)CS(=O)(=O)N4C3COC3)n[nH]2)C1 ; [2H]C([2H])([2H])C1c2cc(Nc3cc(C4CCC(OC(=O)NCCC)C4)[nH]n3)cc(F)c2N(C([2H])([2H])[2H])S1(=O)=O ; [2H]C([2H])([2H])N1c2c(F)cc(Nc3cc(C4CCC(OC(=O)N[C@@H](C)CC)C4)[nH]n3)cc2CS1(=O)=O]

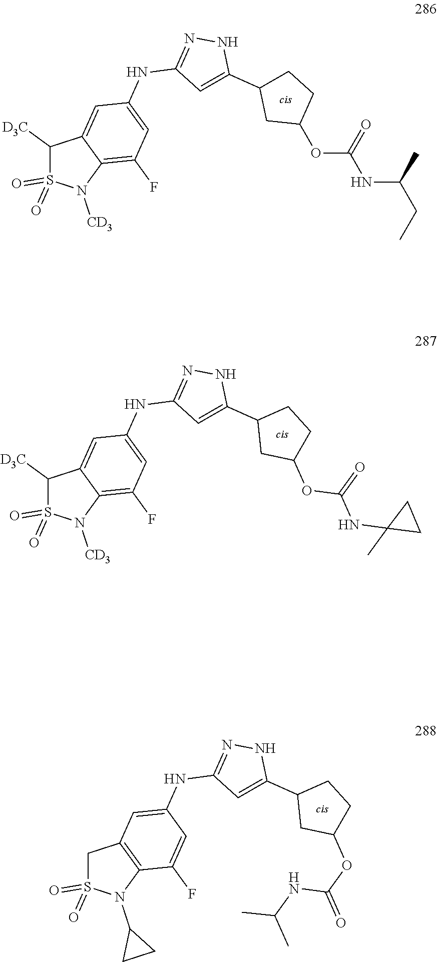 [SMILES: [2H]C([2H])([2H])C1c2cc(Nc3cc(C4CCC(OC(=O)N[C@@H](C)CC)C4)[nH]n3)cc(F)c2N(C([2H])([2H])[2H])S1(=O)=O ; [2H]C([2H])([2H])C1c2cc(Nc3cc(C4CCC(OC(=O)NC5(C)CC5)C4)[nH]n3)cc(F)c2N(C([2H])([2H])[2H])S1(=O)=O ; CC(C)NC(=O)OC1CCC(c2cc(Nc3cc(F)c4c(c3)CS(=O)(=O)N4C3CC3)n[nH]2)C1]

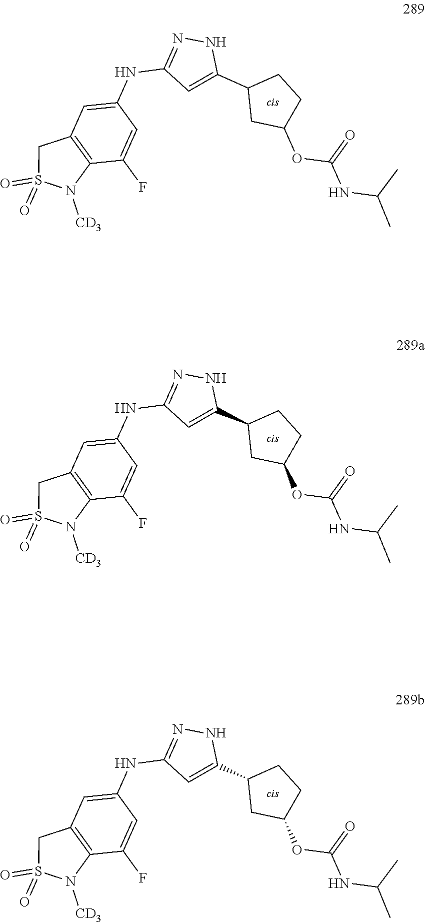 [SMILES: [2H]C([2H])([2H])N1c2c(F)cc(Nc3cc(C4CCC(OC(=O)NC(C)C)C4)[nH]n3)cc2CS1(=O)=O ; [2H]C([2H])([2H])N1c2c(F)cc(Nc3cc([C@H]4CC[C@@H](OC(=O)NC(C)C)C4)[nH]n3)cc2CS1(=O)=O ; [2H]C([2H])([2H])N1c2c(F)cc(Nc3cc([C@@H]4CC[C@H](OC(=O)NC(C)C)C4)[nH]n3)cc2CS1(=O)=O]

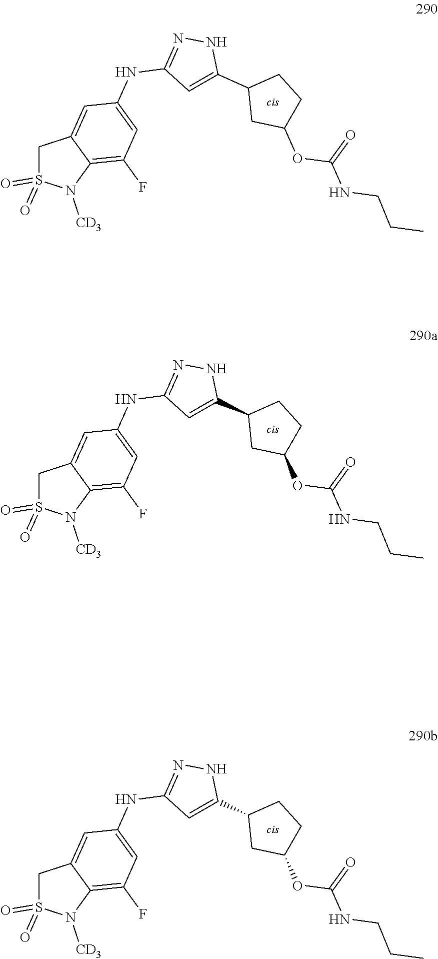 [SMILES: [2H]C([2H])([2H])N1c2c(F)cc(Nc3cc(C4CCC(OC(=O)NCCC)C4)[nH]n3)cc2CS1(=O)=O ; [2H]C([2H])([2H])N1c2c(F)cc(Nc3cc([C@H]4CC[C@@H](OC(=O)NCCC)C4)[nH]n3)cc2CS1(=O)=O ; [2H]C([2H])([2H])N1c2c(F)cc(Nc3cc([C@@H]4CC[C@H](OC(=O)NCCC)C4)[nH]n3)cc2CS1(=O)=O]

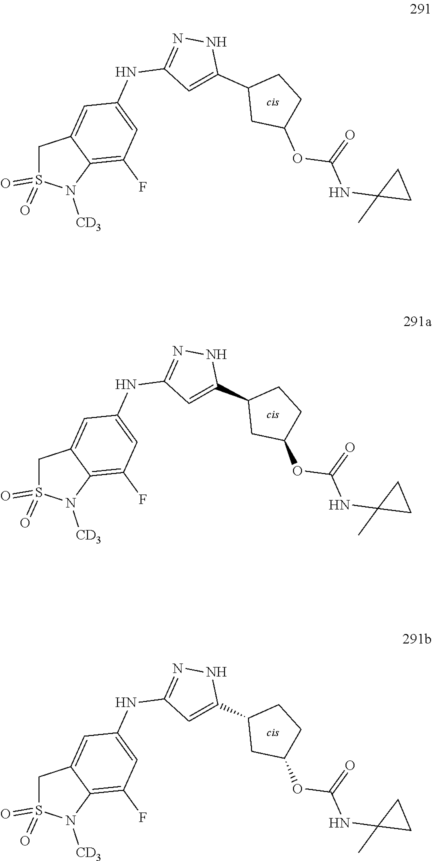 [SMILES: [2H]C([2H])([2H])N1c2c(F)cc(Nc3cc(C4CCC(OC(=O)NC5(C)CC5)C4)[nH]n3)cc2CS1(=O)=O ; [2H]C([2H])([2H])N1c2c(F)cc(Nc3cc([C@H]4CC[C@@H](OC(=O)NC5(C)CC5)C4)[nH]n3)cc2CS1(=O)=O ; [2H]C([2H])([2H])N1c2c(F)cc(Nc3cc([C@@H]4CC[C@H](OC(=O)NC5(C)CC5)C4)[nH]n3)cc2CS1(=O)=O]

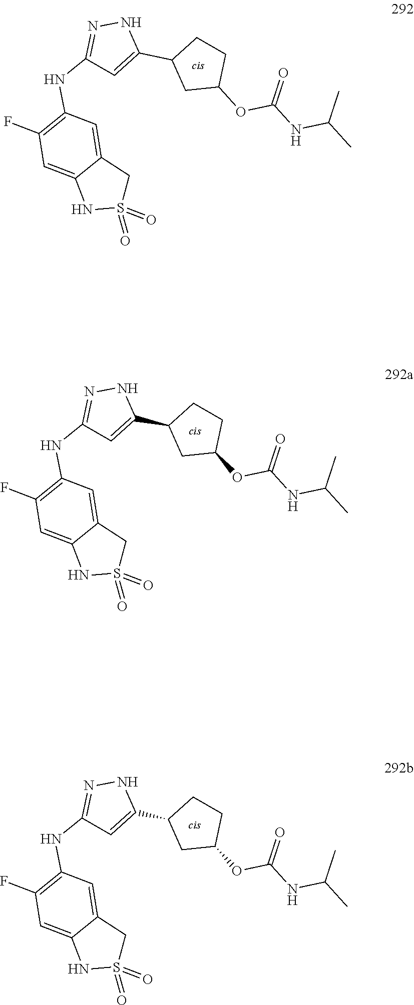 [SMILES: CC(C)NC(=O)OC1CCC(c2cc(Nc3cc4c(cc3F)NS(=O)(=O)C4)n[nH]2)C1 ; CC(C)NC(=O)O[C@@H]1CC[C@H](c2cc(Nc3cc4c(cc3F)NS(=O)(=O)C4)n[nH]2)C1 ; CC(C)NC(=O)O[C@H]1CC[C@@H](c2cc(Nc3cc4c(cc3F)NS(=O)(=O)C4)n[nH]2)C1]

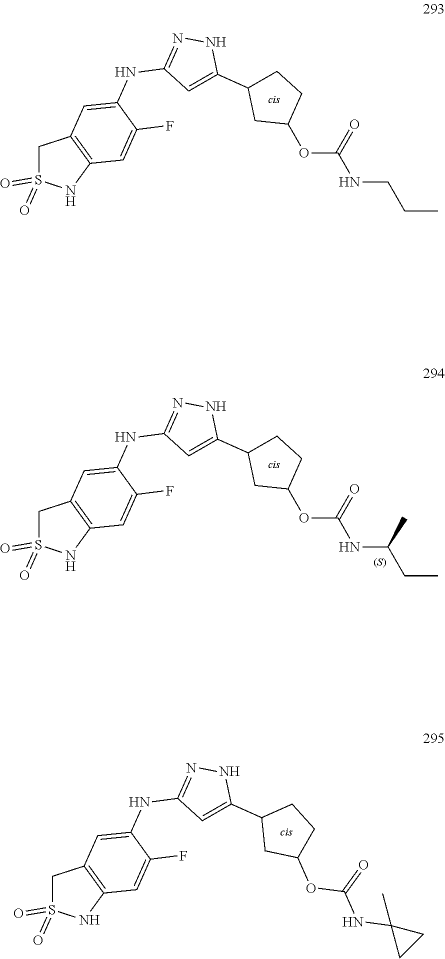

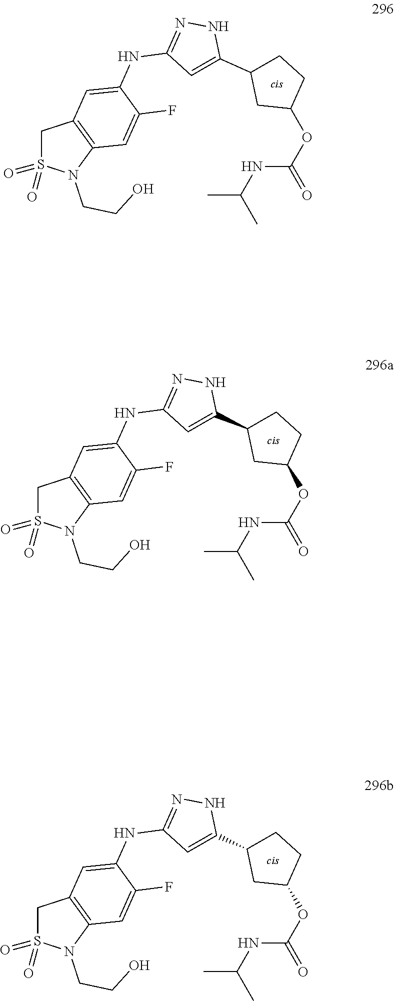 [SMILES: CC(C)NC(=O)OC1CCC(c2cc(Nc3cc4c(cc3F)N(CCO)S(=O)(=O)C4)n[nH]2)C1 ; CC(C)NC(=O)O[C@@H]1CC[C@H](c2cc(Nc3cc4c(cc3F)N(CCO)S(=O)(=O)C4)n[nH]2)C1 ; CC(C)NC(=O)O[C@H]1CC[C@@H](c2cc(Nc3cc4c(cc3F)N(CCO)S(=O)(=O)C4)n[nH]2)C1]

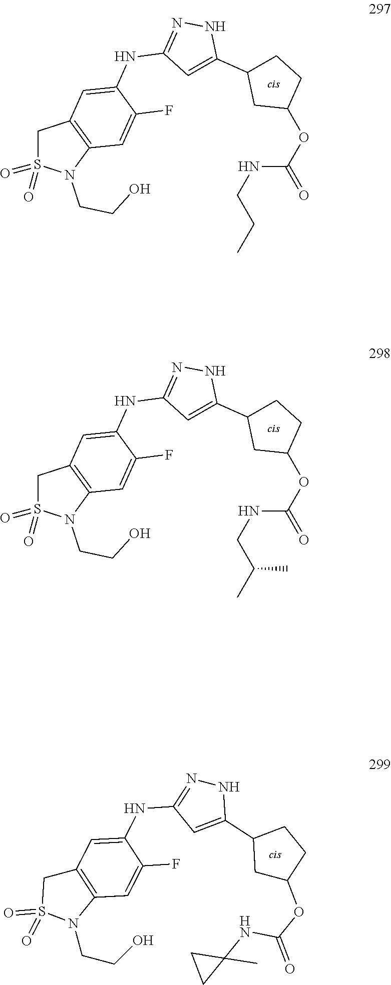

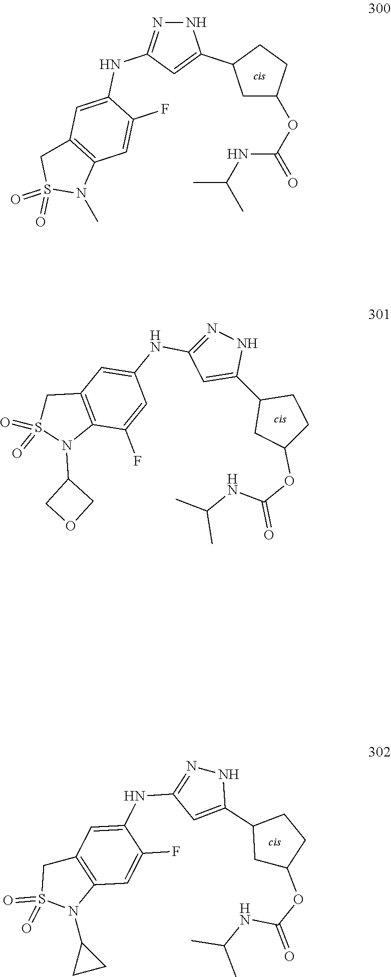 [SMILES: CC(C)NC(=O)OC1CCC(c2cc(Nc3cc4c(cc3F)N(C)S(=O)(=O)C4)n[nH]2)C1 ; CC(C)NC(=O)OC1CCC(c2cc(Nc3cc(F)c4c(c3)CS(=O)(=O)N4C3COC3)n[nH]2)C1 ; CC(C)NC(=O)OC1CCC(c2cc(Nc3cc4c(cc3F)N(C3CC3)S(=O)(=O)C4)n[nH]2)C1]

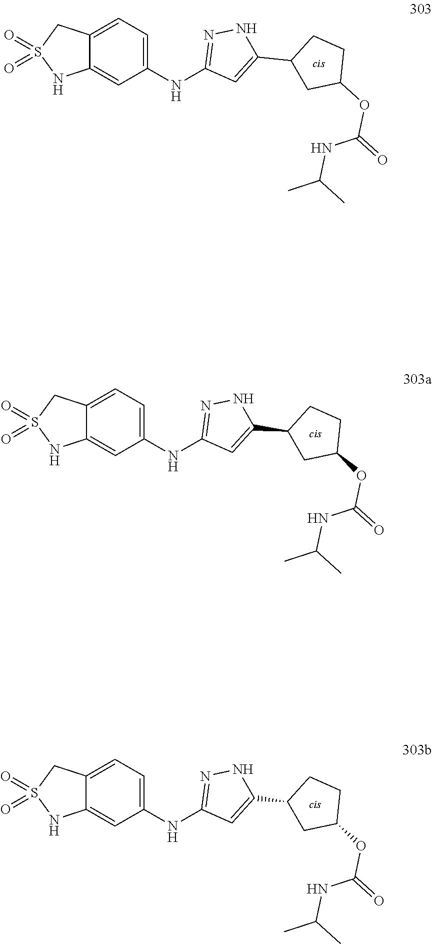 [SMILES: CC(C)NC(=O)OC1CCC(c2cc(Nc3ccc4c(c3)NS(=O)(=O)C4)n[nH]2)C1 ; CC(C)NC(=O)O[C@@H]1CC[C@H](c2cc(Nc3ccc4c(c3)NS(=O)(=O)C4)n[nH]2)C1 ; CC(C)NC(=O)O[C@H]1CC[C@@H](c2cc(Nc3ccc4c(c3)NS(=O)(=O)C4)n[nH]2)C1]

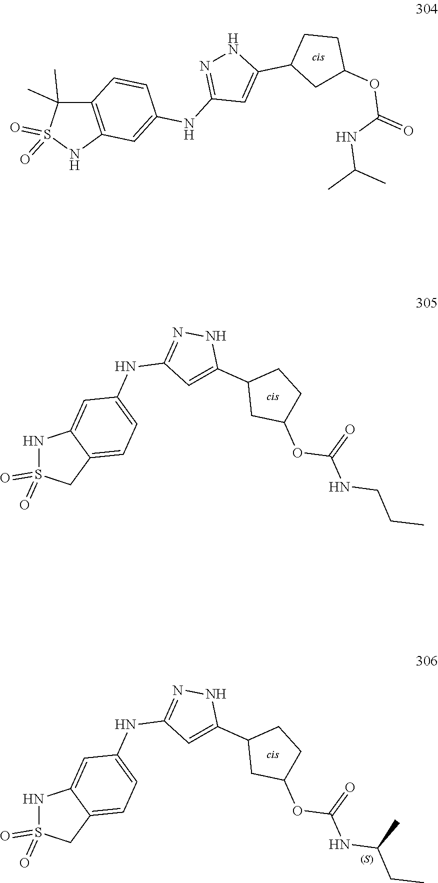

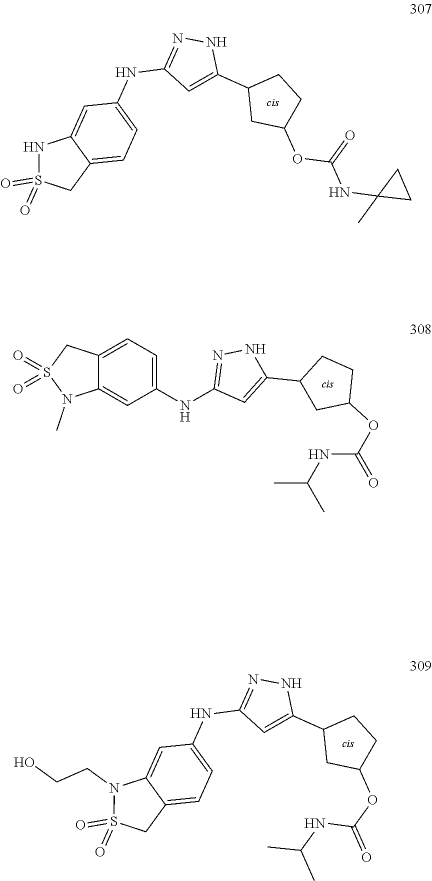 [SMILES: CC1(NC(=O)OC2CCC(c3cc(Nc4ccc5c(c4)NS(=O)(=O)C5)n[nH]3)C2)CC1 ; CC(C)NC(=O)OC1CCC(c2cc(Nc3ccc4c(c3)N(C)S(=O)(=O)C4)n[nH]2)C1 ; CC(C)NC(=O)OC1CCC(c2cc(Nc3ccc4c(c3)N(CCO)S(=O)(=O)C4)n[nH]2)C1]

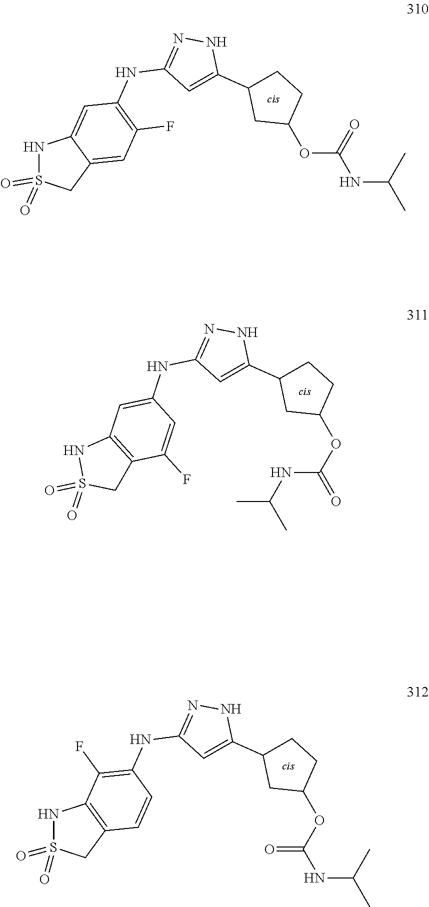 [SMILES: CC(C)NC(=O)OC1CCC(c2cc(Nc3cc4c(cc3F)CS(=O)(=O)N4)n[nH]2)C1 ; CC(C)NC(=O)OC1CCC(c2cc(Nc3cc(F)c4c(c3)NS(=O)(=O)C4)n[nH]2)C1 ; CC(C)NC(=O)OC1CCC(c2cc(Nc3ccc4c(c3F)NS(=O)(=O)C4)n[nH]2)C1]

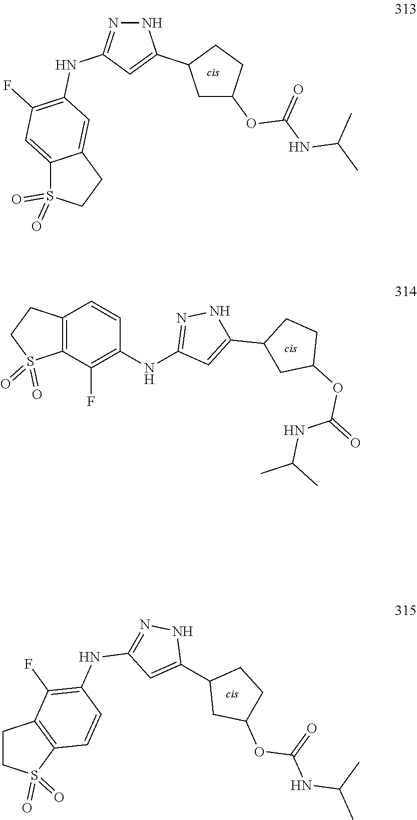 [SMILES: CC(C)NC(=O)OC1CCC(c2cc(Nc3cc4c(cc3F)S(=O)(=O)CC4)n[nH]2)C1 ; CC(C)NC(=O)OC1CCC(c2cc(Nc3ccc4c(c3F)S(=O)(=O)CC4)n[nH]2)C1 ; CC(C)NC(=O)OC1CCC(c2cc(Nc3ccc4c(c3F)CCS4(=O)=O)n[nH]2)C1]

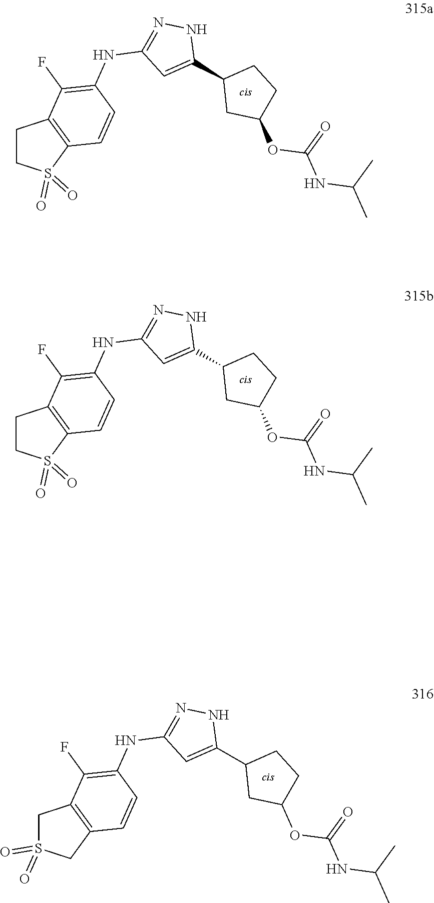 [SMILES: CC(C)NC(=O)O[C@@H]1CC[C@H](c2cc(Nc3ccc4c(c3F)CCS4(=O)=O)n[nH]2)C1 ; CC(C)NC(=O)O[C@H]1CC[C@@H](c2cc(Nc3ccc4c(c3F)CCS4(=O)=O)n[nH]2)C1 ; CC(C)NC(=O)OC1CCC(c2cc(Nc3ccc4c(c3F)CS(=O)(=O)C4)n[nH]2)C1]

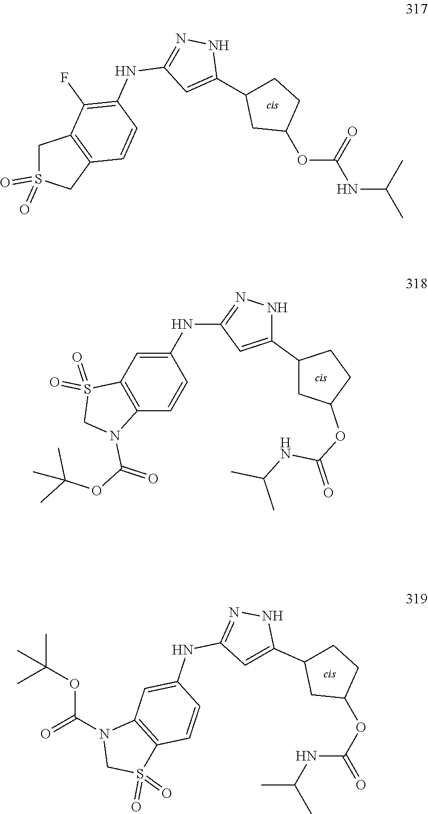

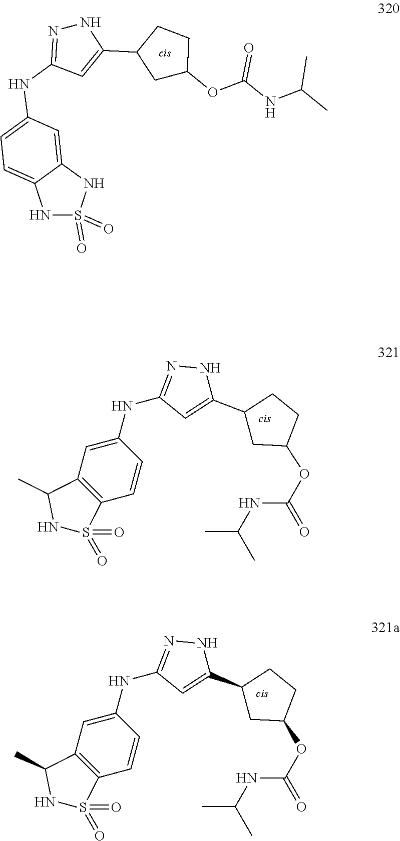

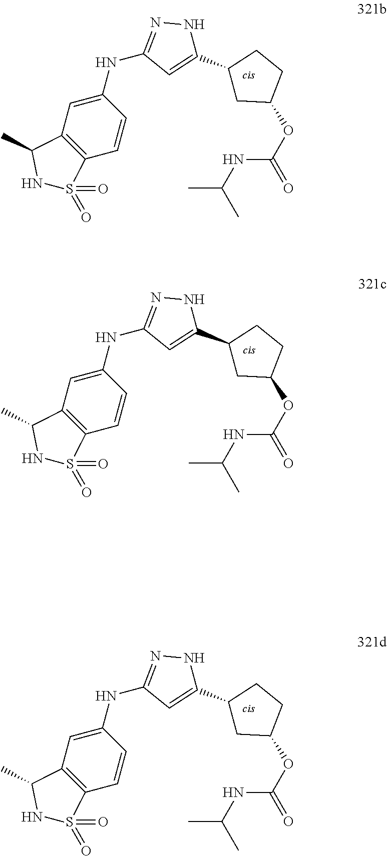 [SMILES: CC(C)NC(=O)O[C@H]1CC[C@@H](c2cc(Nc3ccc4c(c3)[C@H](C)NS4(=O)=O)n[nH]2)C1 ; CC(C)NC(=O)O[C@@H]1CC[C@H](c2cc(Nc3ccc4c(c3)[C@@H](C)NS4(=O)=O)n[nH]2)C1 ; CC(C)NC(=O)O[C@H]1CC[C@@H](c2cc(Nc3ccc4c(c3)[C@@H](C)NS4(=O)=O)n[nH]2)C1]

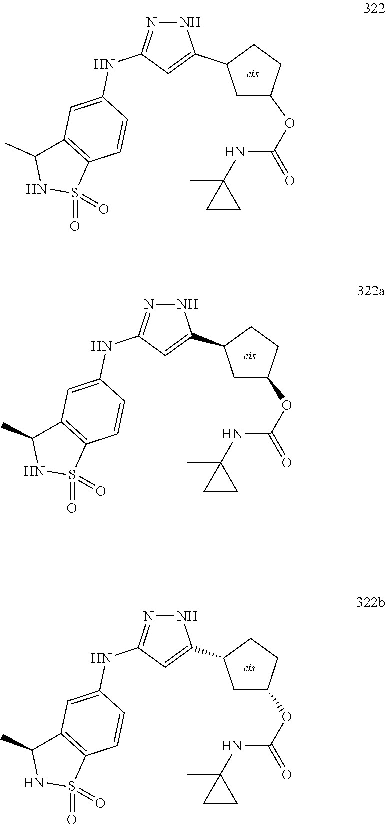 [SMILES: CC1NS(=O)(=O)c2ccc(Nc3cc(C4CCC(OC(=O)NC5(C)CC5)C4)[nH]n3)cc21 ; C[C@@H]1NS(=O)(=O)c2ccc(Nc3cc([C@H]4CC[C@@H](OC(=O)NC5(C)CC5)C4)[nH]n3)cc21 ; C[C@@H]1NS(=O)(=O)c2ccc(Nc3cc([C@@H]4CC[C@H](OC(=O)NC5(C)CC5)C4)[nH]n3)cc21]

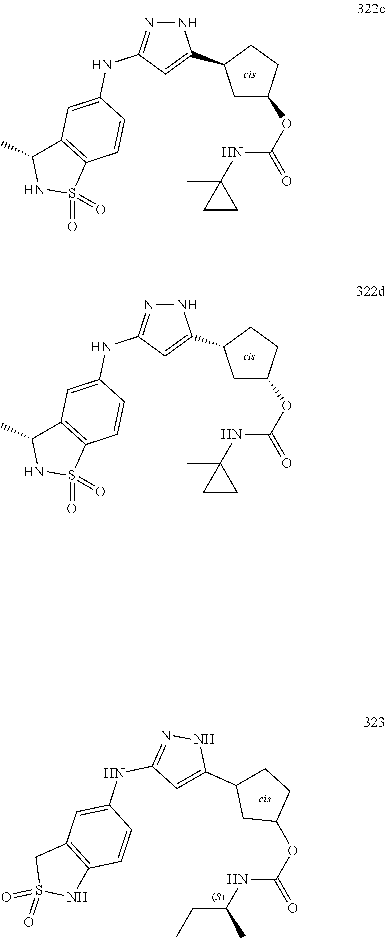

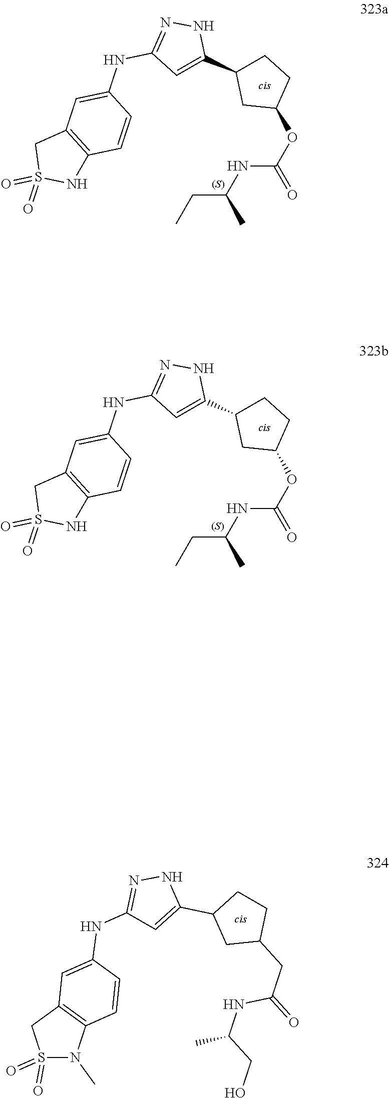

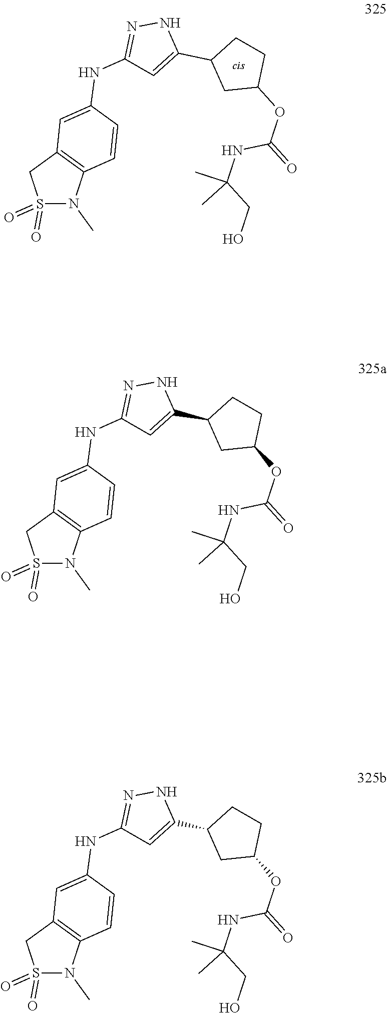 [SMILES: CN1c2ccc(Nc3cc(C4CCC(OC(=O)NC(C)(C)CO)C4)[nH]n3)cc2CS1(=O)=O ; CN1c2ccc(Nc3cc([C@H]4CC[C@@H](OC(=O)NC(C)(C)CO)C4)[nH]n3)cc2CS1(=O)=O ; CN1c2ccc(Nc3cc([C@@H]4CC[C@H](OC(=O)NC(C)(C)CO)C4)[nH]n3)cc2CS1(=O)=O]

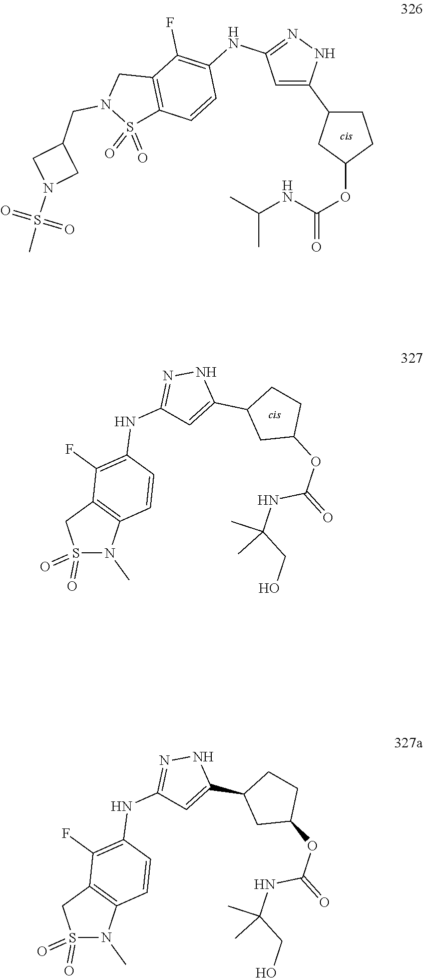 [SMILES: CC(C)NC(=O)OC1CCC(c2cc(Nc3ccc4c(c3F)CN(CC3CN(S(C)(=O)=O)C3)S4(=O)=O)n[nH]2)C1 ; CN1c2ccc(Nc3cc(C4CCC(OC(=O)NC(C)(C)CO)C4)[nH]n3)c(F)c2CS1(=O)=O ; CN1c2ccc(Nc3cc([C@H]4CC[C@@H](OC(=O)NC(C)(C)CO)C4)[nH]n3)c(F)c2CS1(=O)=O]

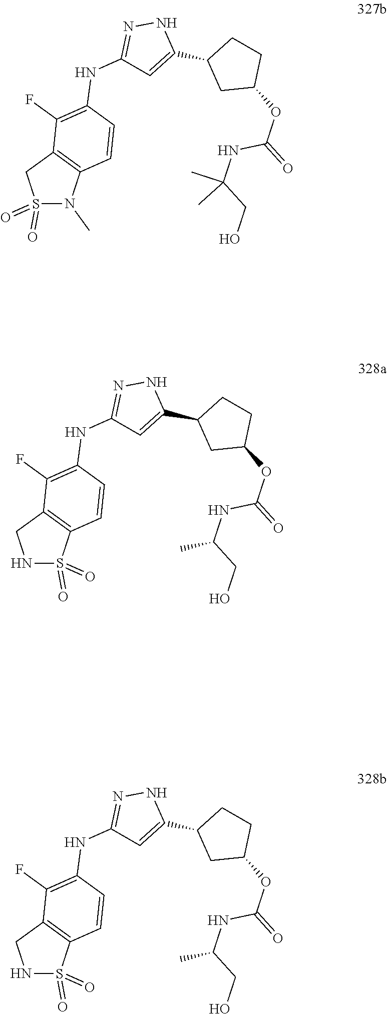 [SMILES: CN1c2ccc(Nc3cc([C@@H]4CC[C@H](OC(=O)NC(C)(C)CO)C4)[nH]n3)c(F)c2CS1(=O)=O ; C[C@@H](CO)NC(=O)O[C@@H]1CC[C@H](c2cc(Nc3ccc4c(c3F)CNS4(=O)=O)n[nH]2)C1 ; C[C@@H](CO)NC(=O)O[C@H]1CC[C@@H](c2cc(Nc3ccc4c(c3F)CNS4(=O)=O)n[nH]2)C1]

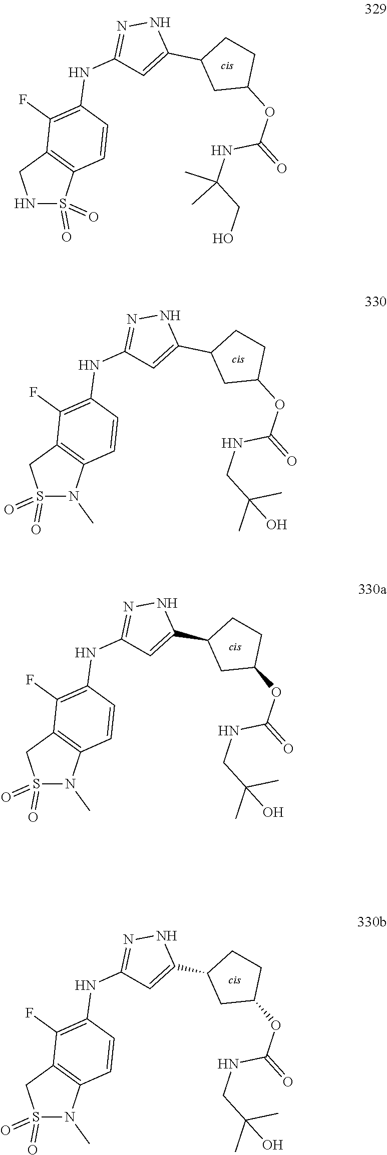 [SMILES: CC(C)(CO)NC(=O)OC1CCC(c2cc(Nc3ccc4c(c3F)CNS4(=O)=O)n[nH]2)C1 ; CN1c2ccc(Nc3cc(C4CCC(OC(=O)NCC(C)(C)O)C4)[nH]n3)c(F)c2CS1(=O)=O ; CN1c2ccc(Nc3cc([C@H]4CC[C@@H](OC(=O)NCC(C)(C)O)C4)[nH]n3)c(F)c2CS1(=O)=O ; CN1c2ccc(Nc3cc([C@@H]4CC[C@H](OC(=O)NCC(C)(C)O)C4)[nH]n3)c(F)c2CS1(=O)=O]

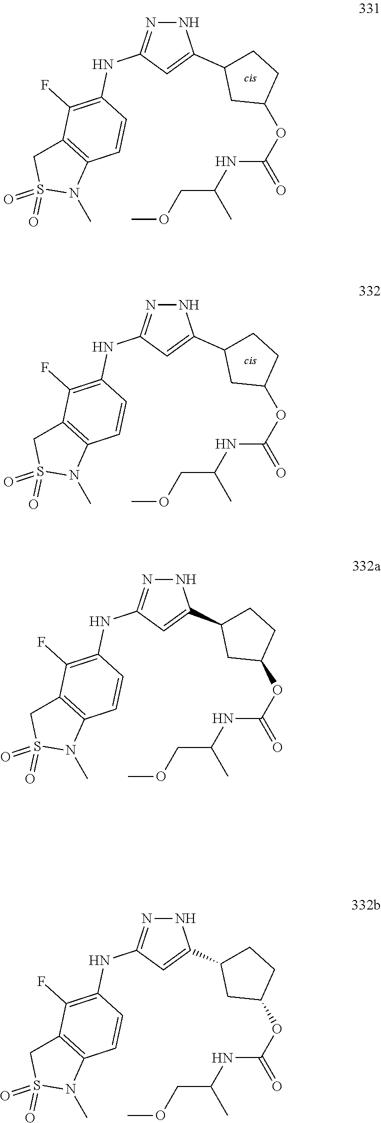 [SMILES: COCC(C)NC(=O)OC1CCC(c2cc(Nc3ccc4c(c3F)CS(=O)(=O)N4C)n[nH]2)C1 ; COCC(C)NC(=O)OC1CCC(c2cc(Nc3ccc4c(c3F)CS(=O)(=O)N4C)n[nH]2)C1 ; COCC(C)NC(=O)O[C@@H]1CC[C@H](c2cc(Nc3ccc4c(c3F)CS(=O)(=O)N4C)n[nH]2)C1 ; COCC(C)NC(=O)O[C@H]1CC[C@@H](c2cc(Nc3ccc4c(c3F)CS(=O)(=O)N4C)n[nH]2)C1]

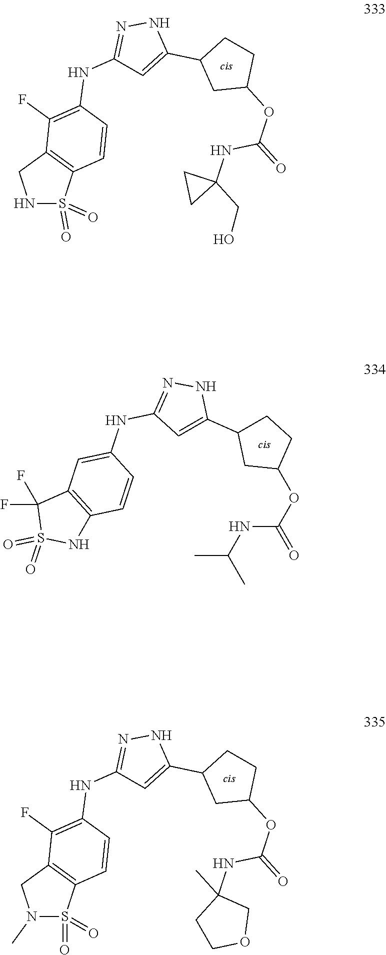 [SMILES: O=C(NC1(CO)CC1)OC1CCC(c2cc(Nc3ccc4c(c3F)CNS4(=O)=O)n[nH]2)C1 ; CC(C)NC(=O)OC1CCC(c2cc(Nc3ccc4c(c3)C(F)(F)S(=O)(=O)N4)n[nH]2)C1 ; CN1Cc2c(ccc(Nc3cc(C4CCC(OC(=O)NC5(C)CCOC5)C4)[nH]n3)c2F)S1(=O)=O]

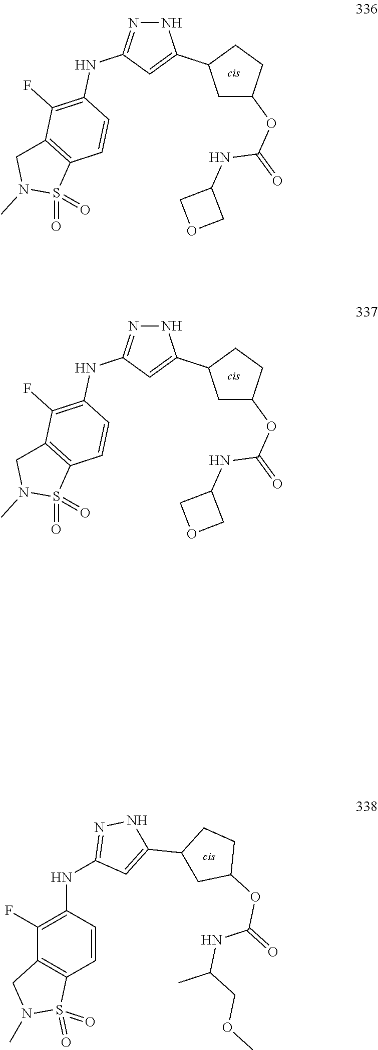 [SMILES: CN1Cc2c(ccc(Nc3cc(C4CCC(OC(=O)NC5COC5)C4)[nH]n3)c2F)S1(=O)=O ; CN1Cc2c(ccc(Nc3cc(C4CCC(OC(=O)NC5COC5)C4)[nH]n3)c2F)S1(=O)=O ; COCC(C)NC(=O)OC1CCC(c2cc(Nc3ccc4c(c3F)CN(C)S4(=O)=O)n[nH]2)C1]

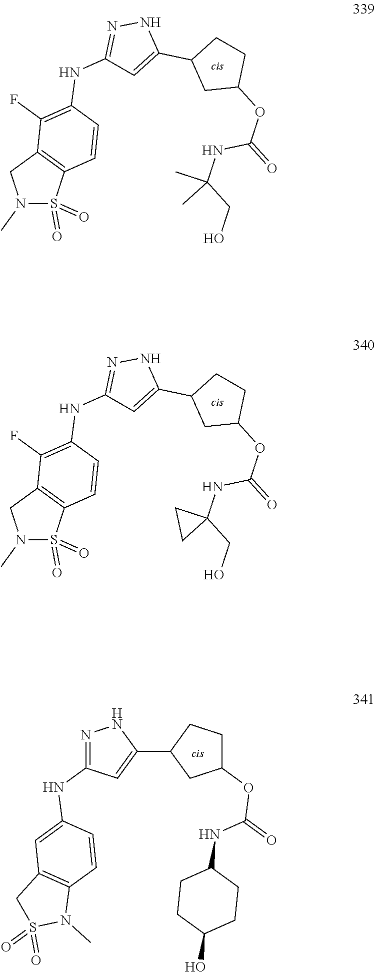

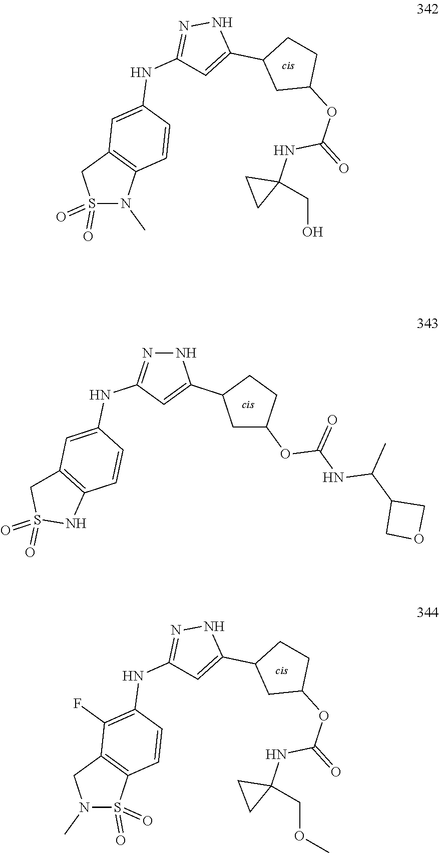 [SMILES: CN1c2ccc(Nc3cc(C4CCC(OC(=O)NC5(CO)CC5)C4)[nH]n3)cc2CS1(=O)=O ; CC(NC(=O)OC1CCC(c2cc(Nc3ccc4c(c3)CS(=O)(=O)N4)n[nH]2)C1)C1COC1 ; COCC1(NC(=O)OC2CCC(c3cc(Nc4ccc5c(c4F)CN(C)S5(=O)=O)n[nH]3)C2)CC1]

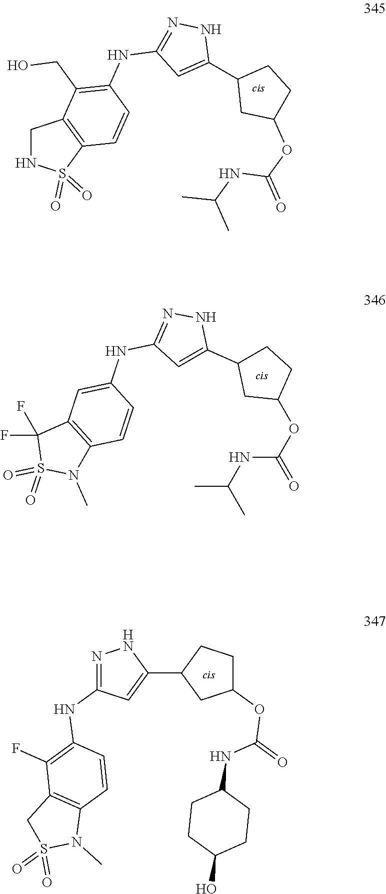

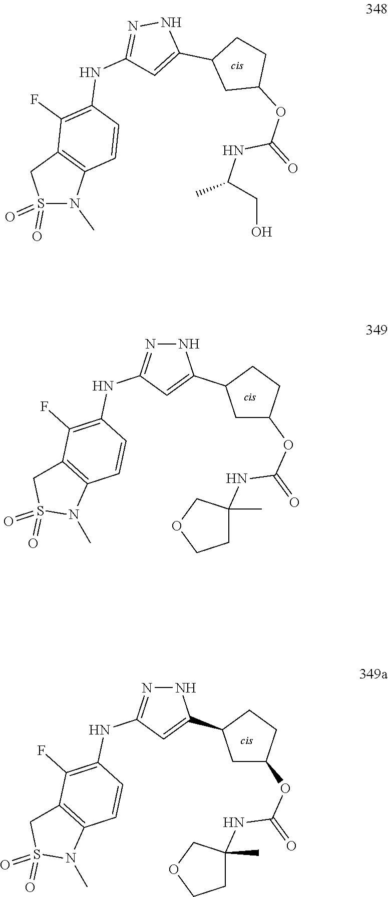 [SMILES: C[C@@H](CO)NC(=O)OC1CCC(c2cc(Nc3ccc4c(c3F)CS(=O)(=O)N4C)n[nH]2)C1 ; CN1c2ccc(Nc3cc(C4CCC(OC(=O)NC5(C)CCOC5)C4)[nH]n3)c(F)c2CS1(=O)=O ; CN1c2ccc(Nc3cc([C@H]4CC[C@@H](OC(=O)N[C@]5(C)CCOC5)C4)[nH]n3)c(F)c2CS1(=O)=O]

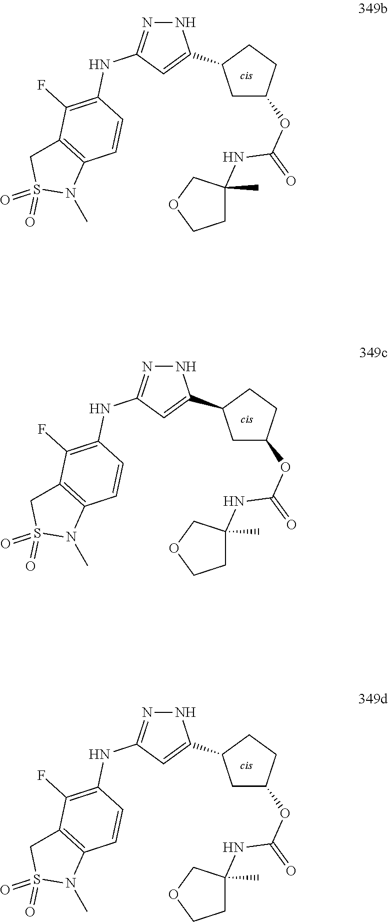 [SMILES: CN1c2ccc(Nc3cc([C@@H]4CC[C@H](OC(=O)N[C@]5(C)CCOC5)C4)[nH]n3)c(F)c2CS1(=O)=O ; CN1c2ccc(Nc3cc([C@H]4CC[C@@H](OC(=O)N[C@@]5(C)CCOC5)C4)[nH]n3)c(F)c2CS1(=O)=O ; CN1c2ccc(Nc3cc([C@@H]4CC[C@H](OC(=O)N[C@@]5(C)CCOC5)C4)[nH]n3)c(F)c2CS1(=O)=O]

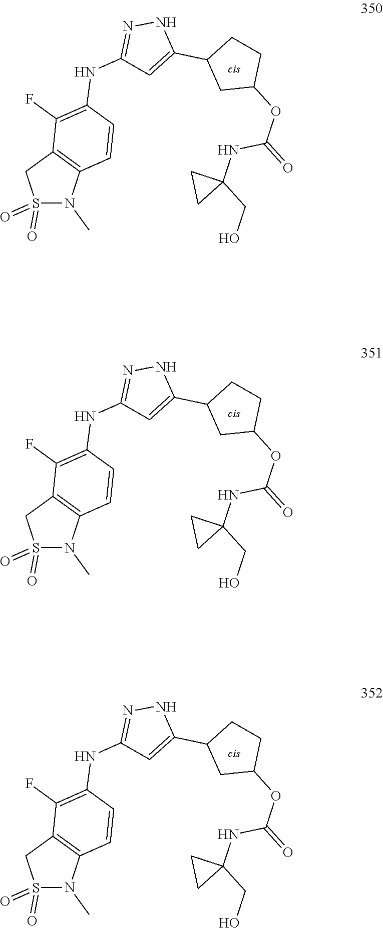 [SMILES: CN1c2ccc(Nc3cc(C4CCC(OC(=O)NC5(CO)CC5)C4)[nH]n3)c(F)c2CS1(=O)=O ; CN1c2ccc(Nc3cc(C4CCC(OC(=O)NC5(CO)CC5)C4)[nH]n3)c(F)c2CS1(=O)=O ; CN1c2ccc(Nc3cc(C4CCC(OC(=O)NC5(CO)CC5)C4)[nH]n3)c(F)c2CS1(=O)=O]

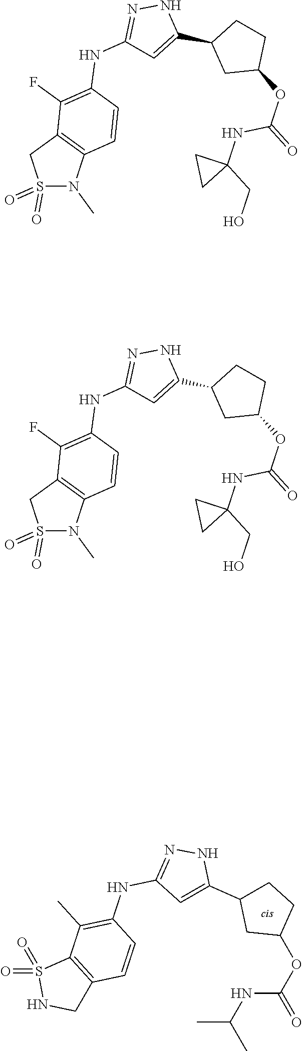 [SMILES: CN1c2ccc(Nc3cc([C@H]4CC[C@@H](OC(=O)NC5(CO)CC5)C4)[nH]n3)c(F)c2CS1(=O)=O ; CN1c2ccc(Nc3cc([C@@H]4CC[C@H](OC(=O)NC5(CO)CC5)C4)[nH]n3)c(F)c2CS1(=O)=O ; Cc1c(Nc2cc(C3CCC(OC(=O)NC(C)C)C3)[nH]n2)ccc2c1S(=O)(=O)NC2]

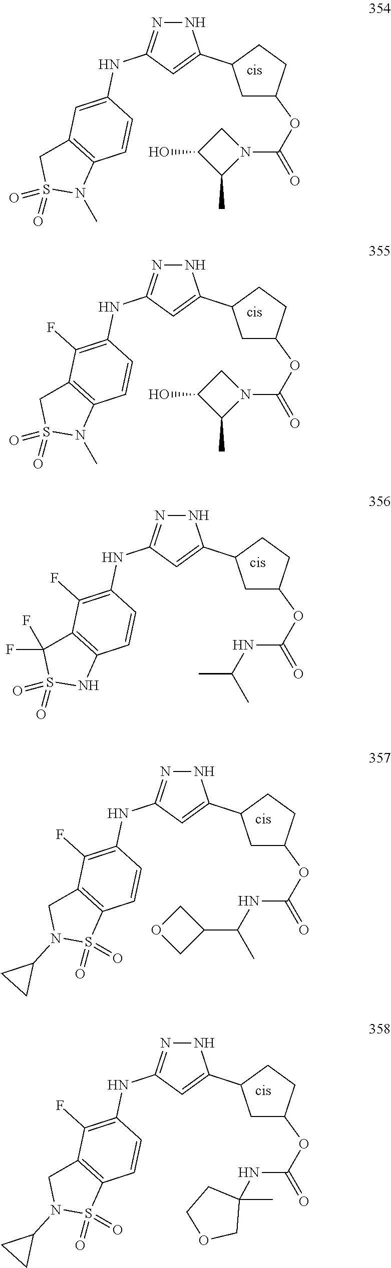 [SMILES: C[C@H]1[C@H](O)CN1C(=O)OC1CCC(c2cc(Nc3ccc4c(c3)CS(=O)(=O)N4C)n[nH]2)C1 ; C[C@H]1[C@H](O)CN1C(=O)OC1CCC(c2cc(Nc3ccc4c(c3F)CS(=O)(=O)N4C)n[nH]2)C1 ; CC(C)NC(=O)OC1CCC(c2cc(Nc3ccc4c(c3F)C(F)(F)S(=O)(=O)N4)n[nH]2)C1 ; CC(NC(=O)OC1CCC(c2cc(Nc3ccc4c(c3F)CN(C3CC3)S4(=O)=O)n[nH]2)C1)C1COC1 ; CC1(NC(=O)OC2CCC(c3cc(Nc4ccc5c(c4F)CN(C4CC4)S5(=O)=O)n[nH]3)C2)CCOC1]

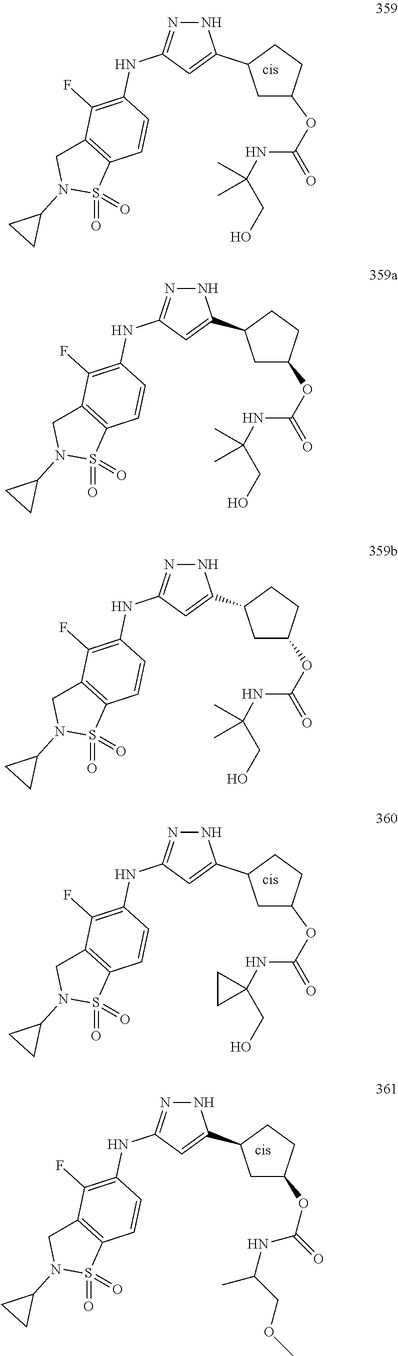 [SMILES: CC(C)(CO)NC(=O)OC1CCC(c2cc(Nc3ccc4c(c3F)CN(C3CC3)S4(=O)=O)n[nH]2)C1 ; CC(C)(CO)NC(=O)O[C@@H]1CC[C@H](c2cc(Nc3ccc4c(c3F)CN(C3CC3)S4(=O)=O)n[nH]2)C1 ; CC(C)(CO)NC(=O)O[C@H]1CC[C@@H](c2cc(Nc3ccc4c(c3F)CN(C3CC3)S4(=O)=O)n[nH]2)C1 ; O=C(NC1(CO)CC1)OC1CCC(c2cc(Nc3ccc4c(c3F)CN(C3CC3)S4(=O)=O)n[nH]2)C1 ; COCC(C)NC(=O)O[C@@H]1CC[C@H](c2cc(Nc3ccc4c(c3F)CN(C3CC3)S4(=O)=O)n[nH]2)C1]

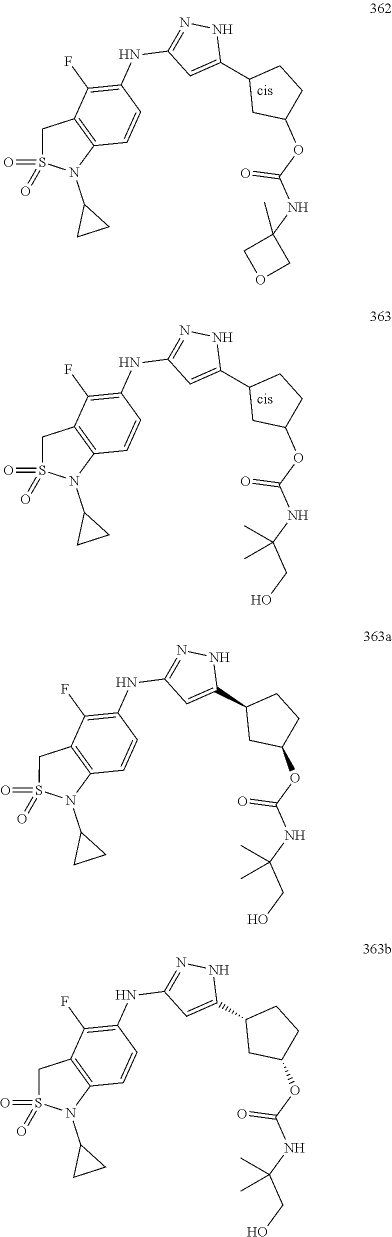 [SMILES: CC1(NC(=O)OC2CCC(c3cc(Nc4ccc5c(c4F)CS(=O)(=O)N5C4CC4)n[nH]3)C2)COC1 ; CC(C)(CO)NC(=O)OC1CCC(c2cc(Nc3ccc4c(c3F)CS(=O)(=O)N4C3CC3)n[nH]2)C1 ; CC(C)(CO)NC(=O)O[C@@H]1CC[C@H](c2cc(Nc3ccc4c(c3F)CS(=O)(=O)N4C3CC3)n[nH]2)C1 ; CC(C)(CO)NC(=O)O[C@H]1CC[C@@H](c2cc(Nc3ccc4c(c3F)CS(=O)(=O)N4C3CC3)n[nH]2)C1]

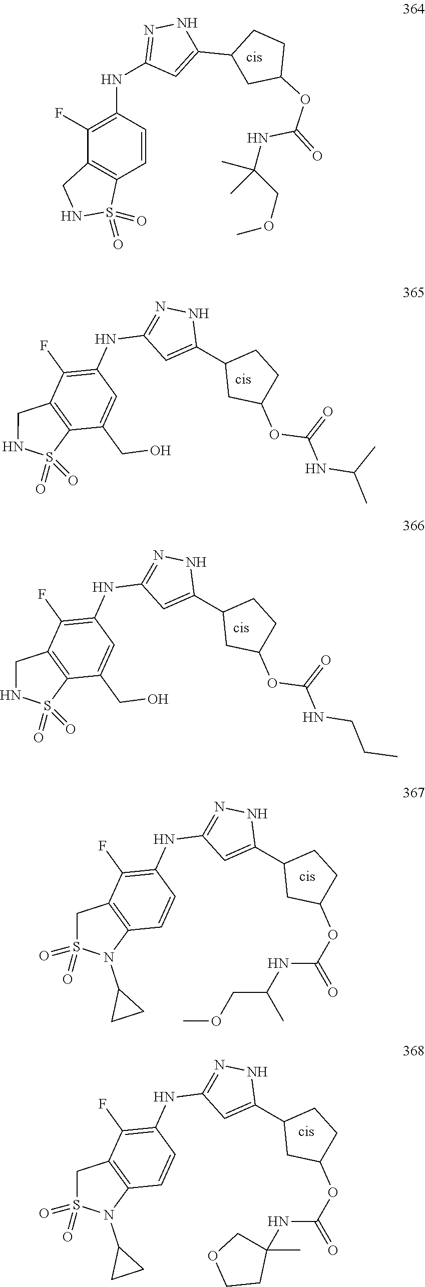

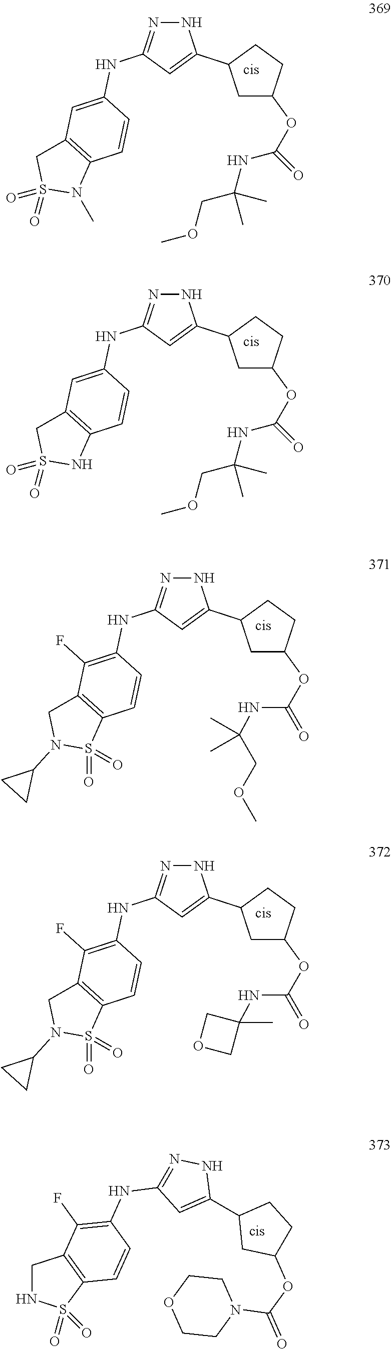 [SMILES: COCC(C)(C)NC(=O)OC1CCC(c2cc(Nc3ccc4c(c3)CS(=O)(=O)N4C)n[nH]2)C1 ; COCC(C)(C)NC(=O)OC1CCC(c2cc(Nc3ccc4c(c3)CS(=O)(=O)N4)n[nH]2)C1 ; COCC(C)(C)NC(=O)OC1CCC(c2cc(Nc3ccc4c(c3F)CN(C3CC3)S4(=O)=O)n[nH]2)C1 ; CC1(NC(=O)OC2CCC(c3cc(Nc4ccc5c(c4F)CN(C4CC4)S5(=O)=O)n[nH]3)C2)COC1 ; O=C(OC1CCC(c2cc(Nc3ccc4c(c3F)CNS4(=O)=O)n[nH]2)C1)N1CCOCC1]

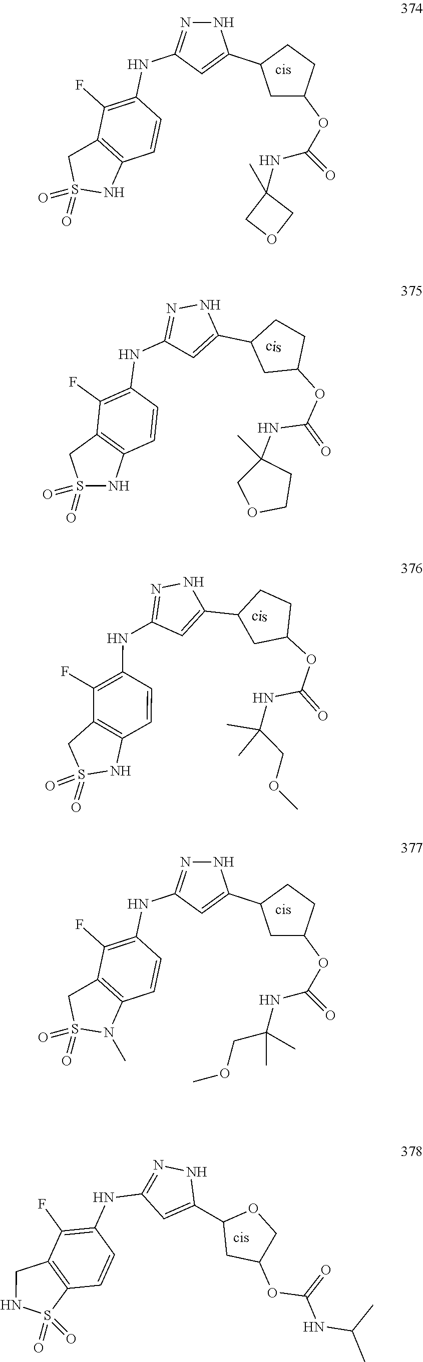 [SMILES: CC1(NC(=O)OC2CCC(c3cc(Nc4ccc5c(c4F)CS(=O)(=O)N5)n[nH]3)C2)COC1 ; CC1(NC(=O)OC2CCC(c3cc(Nc4ccc5c(c4F)CS(=O)(=O)N5)n[nH]3)C2)CCOC1 ; COCC(C)(C)NC(=O)OC1CCC(c2cc(Nc3ccc4c(c3F)CS(=O)(=O)N4)n[nH]2)C1 ; COCC(C)(C)NC(=O)OC1CCC(c2cc(Nc3ccc4c(c3F)CS(=O)(=O)N4C)n[nH]2)C1 ; CC(C)NC(=O)OC1COC(c2cc(Nc3ccc4c(c3F)CNS4(=O)=O)n[nH]2)C1]

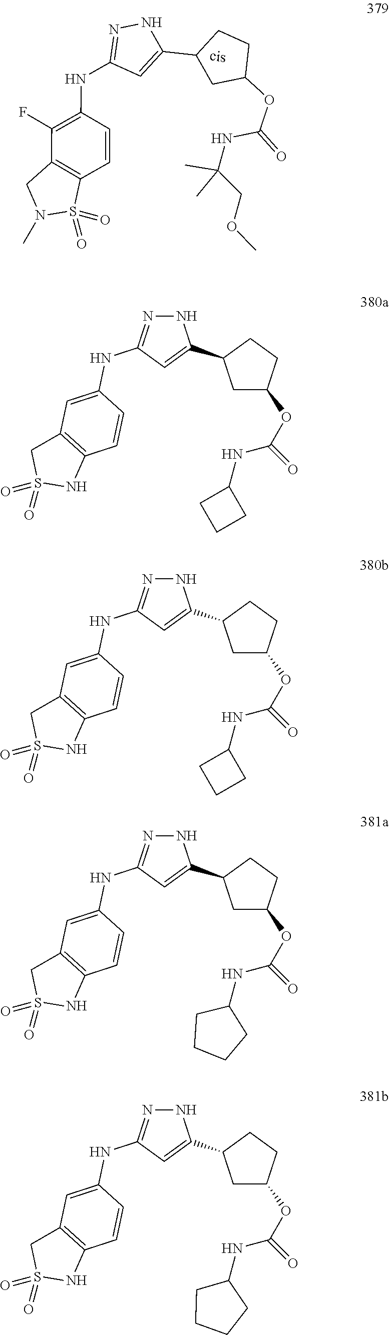 [SMILES: COCC(C)(C)NC(=O)OC1CCC(c2cc(Nc3ccc4c(c3F)CN(C)S4(=O)=O)n[nH]2)C1 ; O=C(NC1CCC1)O[C@@H]1CC[C@H](c2cc(Nc3ccc4c(c3)CS(=O)(=O)N4)n[nH]2)C1 ; O=C(NC1CCC1)O[C@H]1CC[C@@H](c2cc(Nc3ccc4c(c3)CS(=O)(=O)N4)n[nH]2)C1 ; O=C(NC1CCCC1)O[C@@H]1CC[C@H](c2cc(Nc3ccc4c(c3)CS(=O)(=O)N4)n[nH]2)C1 ; O=C(NC1CCCC1)O[C@H]1CC[C@@H](c2cc(Nc3ccc4c(c3)CS(=O)(=O)N4)n[nH]2)C1]

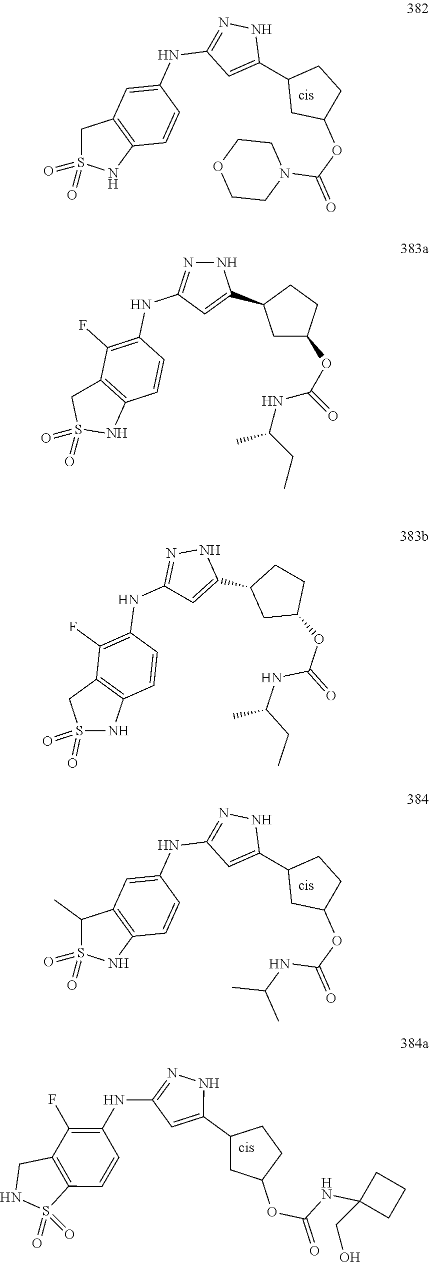 [SMILES: O=C(OC1CCC(c2cc(Nc3ccc4c(c3)CS(=O)(=O)N4)n[nH]2)C1)N1CCOCC1 ; CC[C@H](C)NC(=O)O[C@@H]1CC[C@H](c2cc(Nc3ccc4c(c3F)CS(=O)(=O)N4)n[nH]2)C1 ; CC[C@H](C)NC(=O)O[C@H]1CC[C@@H](c2cc(Nc3ccc4c(c3F)CS(=O)(=O)N4)n[nH]2)C1 ; CC(C)NC(=O)OC1CCC(c2cc(Nc3ccc4c(c3)C(C)S(=O)(=O)N4)n[nH]2)C1 ; O=C(NC1(CO)CCC1)OC1CCC(c2cc(Nc3ccc4c(c3F)CNS4(=O)=O)n[nH]2)C1]

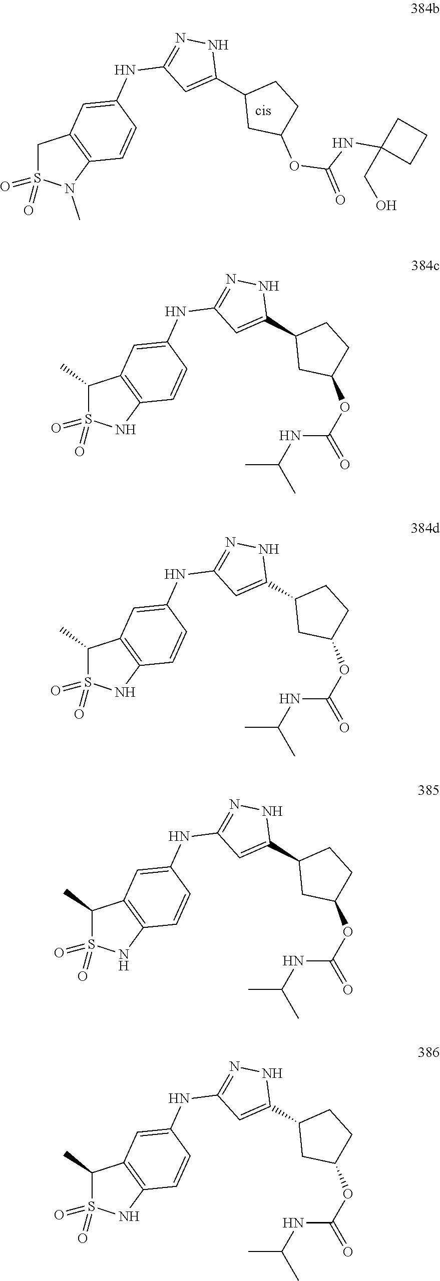 [SMILES: CN1c2ccc(Nc3cc(C4CCC(OC(=O)NC5(CO)CCC5)C4)[nH]n3)cc2CS1(=O)=O ; CC(C)NC(=O)O[C@@H]1CC[C@H](c2cc(Nc3ccc4c(c3)[C@@H](C)S(=O)(=O)N4)n[nH]2)C1 ; CC(C)NC(=O)O[C@H]1CC[C@@H](c2cc(Nc3ccc4c(c3)[C@@H](C)S(=O)(=O)N4)n[nH]2)C1 ; CC(C)NC(=O)O[C@@H]1CC[C@H](c2cc(Nc3ccc4c(c3)[C@H](C)S(=O)(=O)N4)n[nH]2)C1 ; CC(C)NC(=O)O[C@H]1CC[C@@H](c2cc(Nc3ccc4c(c3)[C@H](C)S(=O)(=O)N4)n[nH]2)C1]

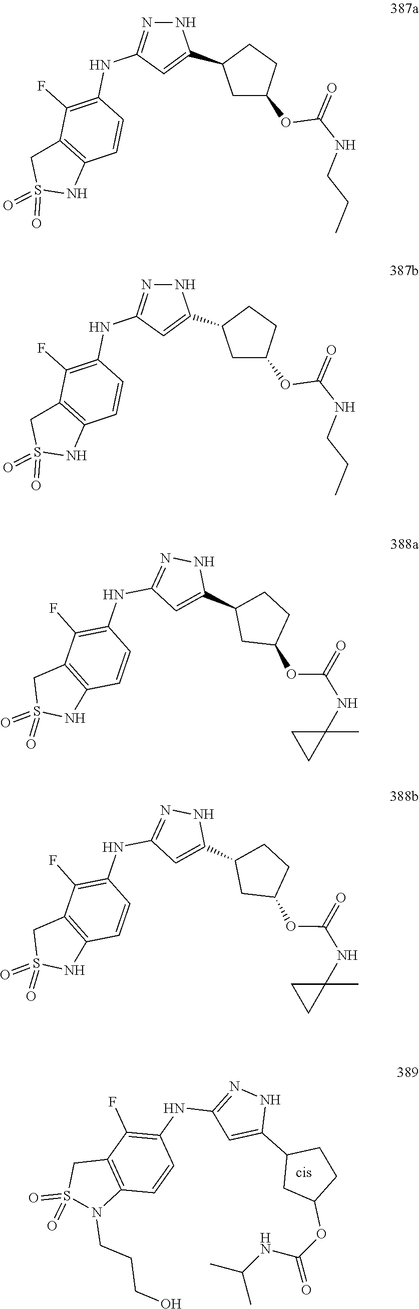 [SMILES: CCCNC(=O)O[C@@H]1CC[C@H](c2cc(Nc3ccc4c(c3F)CS(=O)(=O)N4)n[nH]2)C1 ; CCCNC(=O)O[C@H]1CC[C@@H](c2cc(Nc3ccc4c(c3F)CS(=O)(=O)N4)n[nH]2)C1 ; CC1(NC(=O)O[C@@H]2CC[C@H](c3cc(Nc4ccc5c(c4F)CS(=O)(=O)N5)n[nH]3)C2)CC1 ; CC1(NC(=O)O[C@H]2CC[C@@H](c3cc(Nc4ccc5c(c4F)CS(=O)(=O)N5)n[nH]3)C2)CC1 ; CC(C)NC(=O)OC1CCC(c2cc(Nc3ccc4c(c3F)CS(=O)(=O)N4CCCO)n[nH]2)C1]

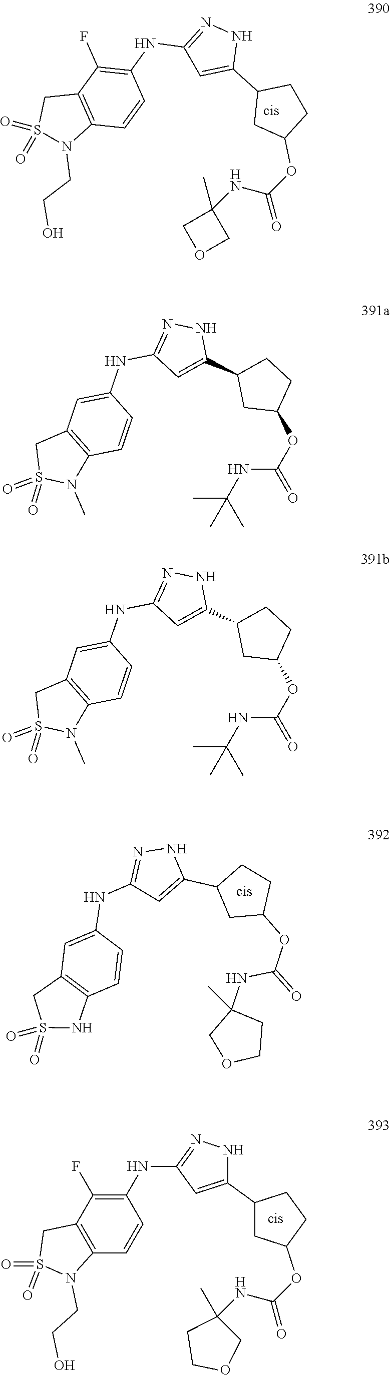 [SMILES: CC1(NC(=O)OC2CCC(c3cc(Nc4ccc5c(c4F)CS(=O)(=O)N5CCO)n[nH]3)C2)COC1 ; CN1c2ccc(Nc3cc([C@H]4CC[C@@H](OC(=O)NC(C)(C)C)C4)[nH]n3)cc2CS1(=O)=O ; CN1c2ccc(Nc3cc([C@@H]4CC[C@H](OC(=O)NC(C)(C)C)C4)[nH]n3)cc2CS1(=O)=O ; CC1(NC(=O)OC2CCC(c3cc(Nc4ccc5c(c4)CS(=O)(=O)N5)n[nH]3)C2)CCOC1 ; CC1(NC(=O)OC2CCC(c3cc(Nc4ccc5c(c4F)CS(=O)(=O)N5CCO)n[nH]3)C2)CCOC1]

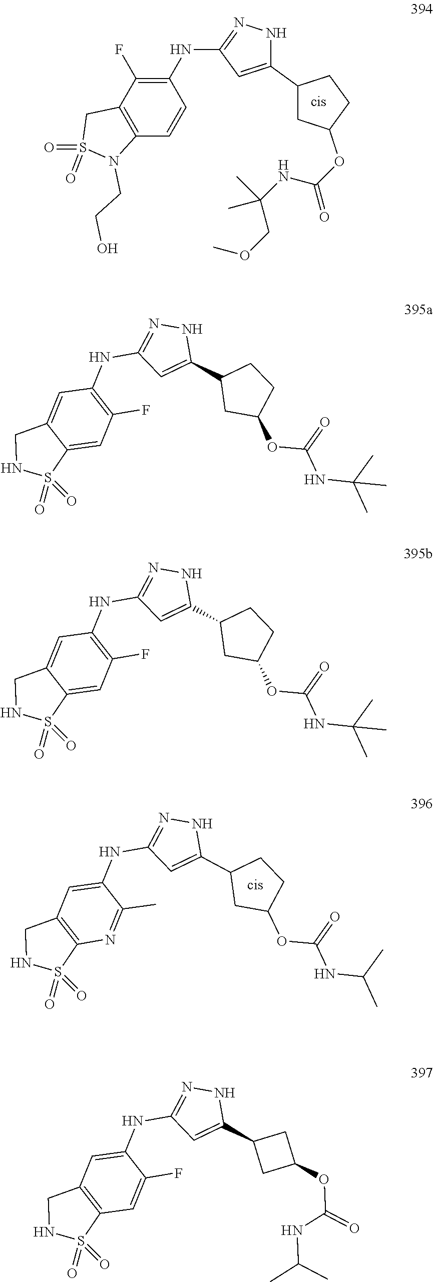

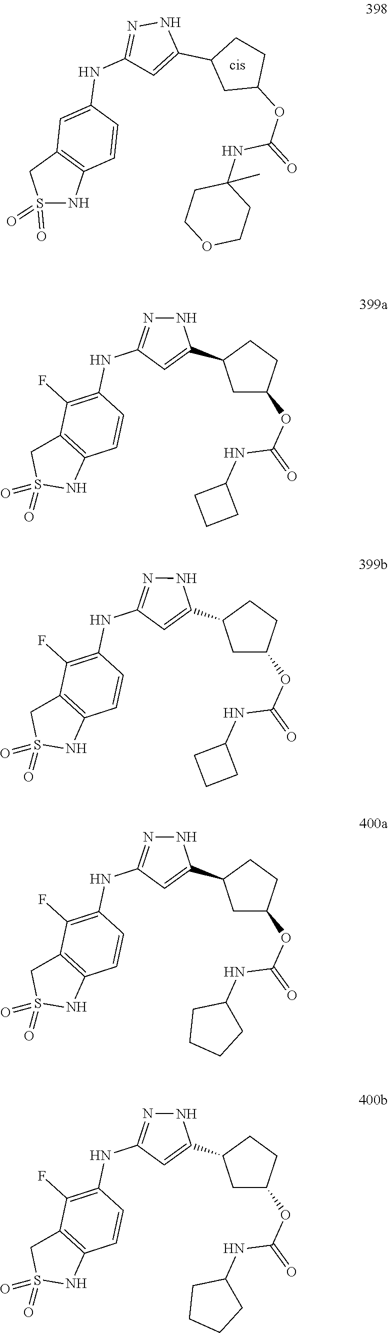 [SMILES: CC1(NC(=O)OC2CCC(c3cc(Nc4ccc5c(c4)CS(=O)(=O)N5)n[nH]3)C2)CCOCC1 ; O=C(NC1CCC1)O[C@@H]1CC[C@H](c2cc(Nc3ccc4c(c3F)CS(=O)(=O)N4)n[nH]2)C1 ; O=C(NC1CCC1)O[C@H]1CC[C@@H](c2cc(Nc3ccc4c(c3F)CS(=O)(=O)N4)n[nH]2)C1 ; O=C(NC1CCCC1)O[C@@H]1CC[C@H](c2cc(Nc3ccc4c(c3F)CS(=O)(=O)N4)n[nH]2)C1 ; O=C(NC1CCCC1)O[C@H]1CC[C@@H](c2cc(Nc3ccc4c(c3F)CS(=O)(=O)N4)n[nH]2)C1]

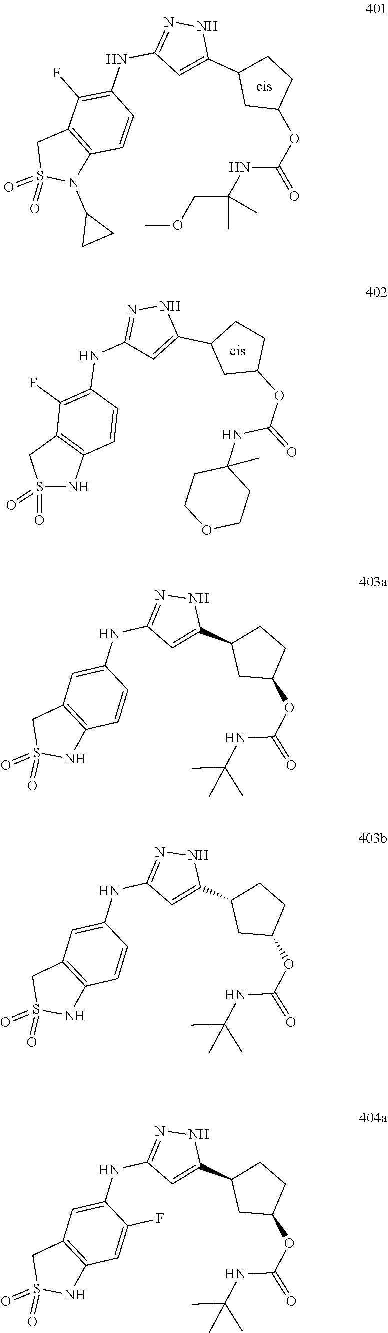 [SMILES: COCC(C)(C)NC(=O)OC1CCC(c2cc(Nc3ccc4c(c3F)CS(=O)(=O)N4C3CC3)n[nH]2)C1 ; CC1(NC(=O)OC2CCC(c3cc(Nc4ccc5c(c4F)CS(=O)(=O)N5)n[nH]3)C2)CCOCC1 ; CC(C)(C)NC(=O)O[C@@H]1CC[C@H](c2cc(Nc3ccc4c(c3)CS(=O)(=O)N4)n[nH]2)C1 ; CC(C)(C)NC(=O)O[C@H]1CC[C@@H](c2cc(Nc3ccc4c(c3)CS(=O)(=O)N4)n[nH]2)C1 ; CC(C)(C)NC(=O)O[C@@H]1CC[C@H](c2cc(Nc3cc4c(cc3F)NS(=O)(=O)C4)n[nH]2)C1]

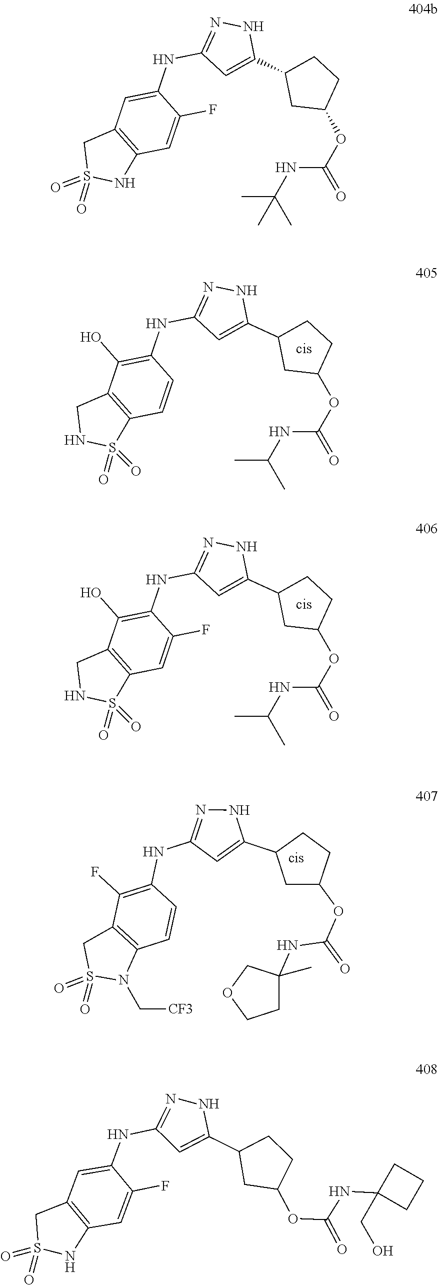 [SMILES: CC(C)(C)NC(=O)O[C@H]1CC[C@@H](c2cc(Nc3cc4c(cc3F)NS(=O)(=O)C4)n[nH]2)C1 ; CC(C)NC(=O)OC1CCC(c2cc(Nc3ccc4c(c3O)CNS4(=O)=O)n[nH]2)C1 ; CC(C)NC(=O)OC1CCC(c2cc(Nc3c(F)cc4c(c3O)CNS4(=O)=O)n[nH]2)C1 ; CC1(NC(=O)OC2CCC(c3cc(Nc4ccc5c(c4F)CS(=O)(=O)N5CC(F)(F)F)n[nH]3)C2)CCOC1 ; O=C(NC1(CO)CCC1)OC1CCC(c2cc(Nc3cc4c(cc3F)NS(=O)(=O)C4)n[nH]2)C1]

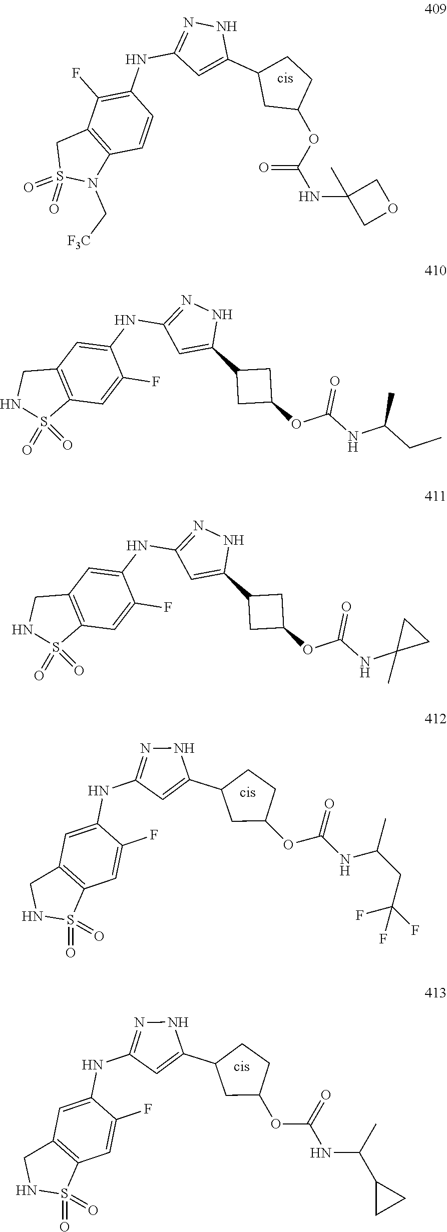

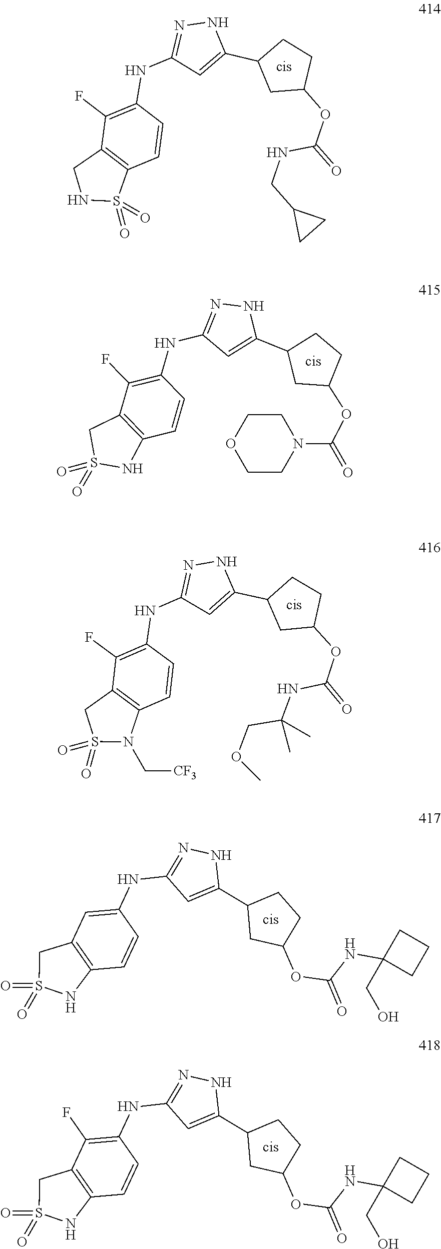 [SMILES: O=C(NCC1CC1)OC1CCC(c2cc(Nc3ccc4c(c3F)CNS4(=O)=O)n[nH]2)C1 ; O=C(OC1CCC(c2cc(Nc3ccc4c(c3F)CS(=O)(=O)N4)n[nH]2)C1)N1CCOCC1 ; COCC(C)(C)NC(=O)OC1CCC(c2cc(Nc3ccc4c(c3F)CS(=O)(=O)N4CC(F)(F)F)n[nH]2)C1 ; O=C(NC1(CO)CCC1)OC1CCC(c2cc(Nc3ccc4c(c3)CS(=O)(=O)N4)n[nH]2)C1 ; O=C(NC1(CO)CCC1)OC1CCC(c2cc(Nc3ccc4c(c3F)CS(=O)(=O)N4)n[nH]2)C1]

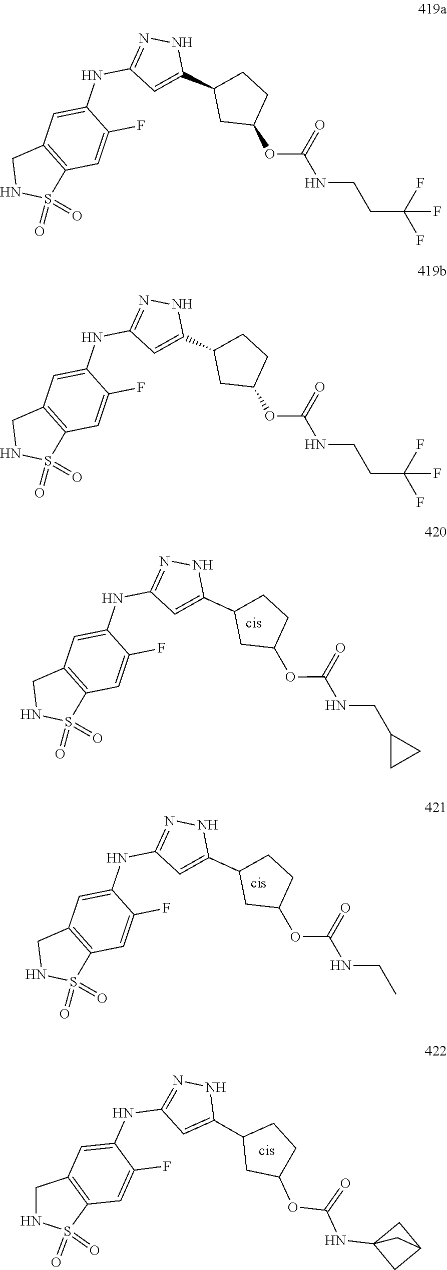 [SMILES: O=C(NCCC(F)(F)F)O[C@@H]1CC[C@H](c2cc(Nc3cc4c(cc3F)S(=O)(=O)NC4)n[nH]2)C1 ; O=C(NCCC(F)(F)F)O[C@H]1CC[C@@H](c2cc(Nc3cc4c(cc3F)S(=O)(=O)NC4)n[nH]2)C1 ; O=C(NCC1CC1)OC1CCC(c2cc(Nc3cc4c(cc3F)S(=O)(=O)NC4)n[nH]2)C1 ; CCNC(=O)OC1CCC(c2cc(Nc3cc4c(cc3F)S(=O)(=O)NC4)n[nH]2)C1 ; O=C(NC12CC(C1)C2)OC1CCC(c2cc(Nc3cc4c(cc3F)S(=O)(=O)NC4)n[nH]2)C1]

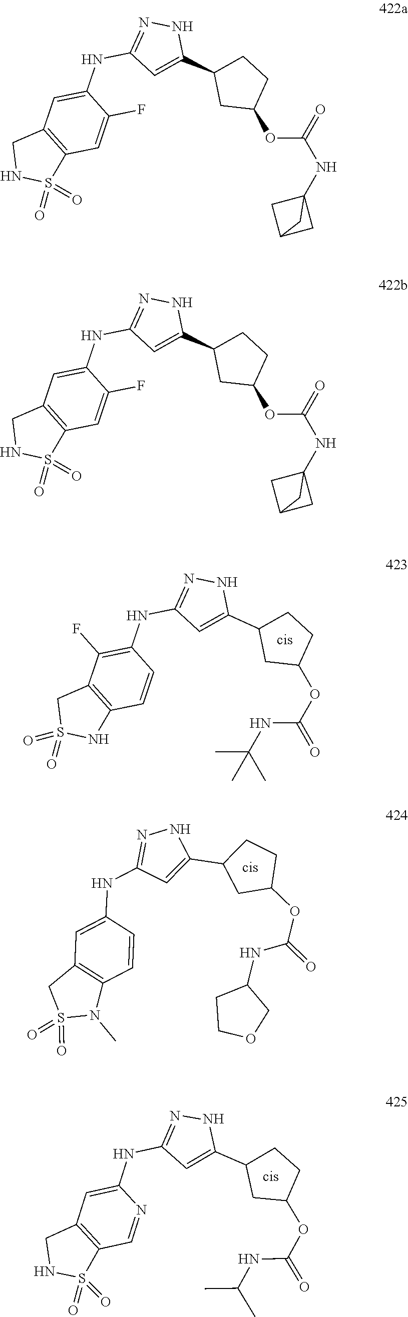 [SMILES: O=C(NC12CC(C1)C2)O[C@@H]1CC[C@H](c2cc(Nc3cc4c(cc3F)S(=O)(=O)NC4)n[nH]2)C1 ; O=C(NC12CC(C1)C2)O[C@@H]1CC[C@H](c2cc(Nc3cc4c(cc3F)S(=O)(=O)NC4)n[nH]2)C1 ; CC(C)(C)NC(=O)OC1CCC(c2cc(Nc3ccc4c(c3F)CS(=O)(=O)N4)n[nH]2)C1 ; CN1c2ccc(Nc3cc(C4CCC(OC(=O)NC5CCOC5)C4)[nH]n3)cc2CS1(=O)=O ; CC(C)NC(=O)OC1CCC(c2cc(Nc3cc4c(cn3)S(=O)(=O)NC4)n[nH]2)C1]

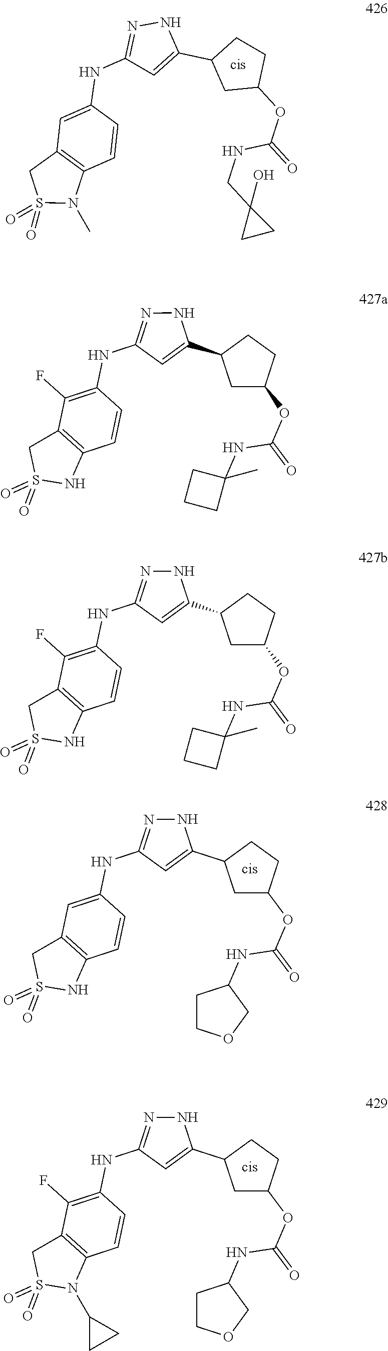 [SMILES: CN1c2ccc(Nc3cc(C4CCC(OC(=O)NCC5(O)CC5)C4)[nH]n3)cc2CS1(=O)=O ; CC1(NC(=O)O[C@@H]2CC[C@H](c3cc(Nc4ccc5c(c4F)CS(=O)(=O)N5)n[nH]3)C2)CCC1 ; CC1(NC(=O)O[C@H]2CC[C@@H](c3cc(Nc4ccc5c(c4F)CS(=O)(=O)N5)n[nH]3)C2)CCC1 ; O=C(NC1CCOC1)OC1CCC(c2cc(Nc3ccc4c(c3)CS(=O)(=O)N4)n[nH]2)C1 ; O=C(NC1CCOC1)OC1CCC(c2cc(Nc3ccc4c(c3F)CS(=O)(=O)N4C3CC3)n[nH]2)C1]

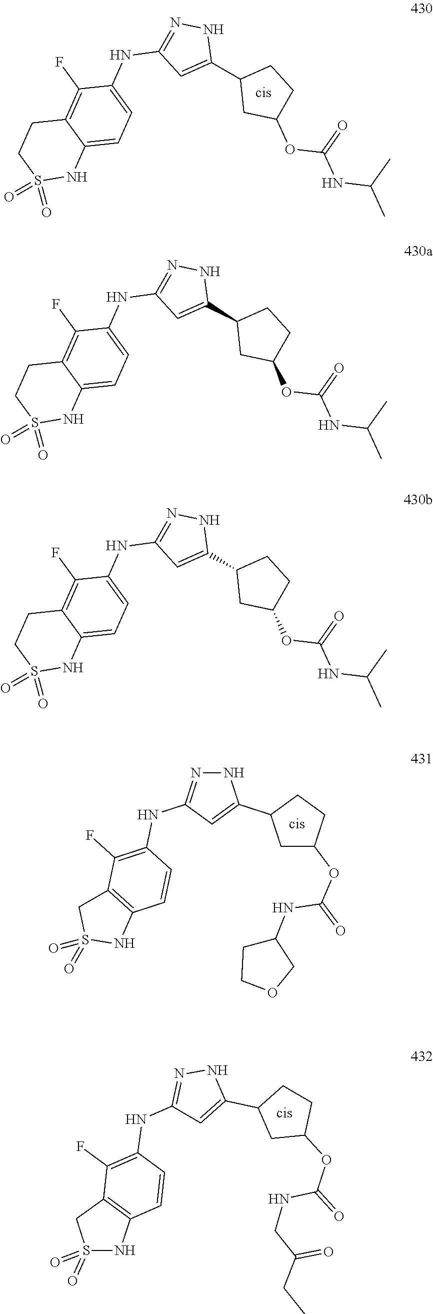 [SMILES: CC(C)NC(=O)OC1CCC(c2cc(Nc3ccc4c(c3F)CCS(=O)(=O)N4)n[nH]2)C1 ; CC(C)NC(=O)O[C@@H]1CC[C@H](c2cc(Nc3ccc4c(c3F)CCS(=O)(=O)N4)n[nH]2)C1 ; CC(C)NC(=O)O[C@H]1CC[C@@H](c2cc(Nc3ccc4c(c3F)CCS(=O)(=O)N4)n[nH]2)C1 ; O=C(NC1CCOC1)OC1CCC(c2cc(Nc3ccc4c(c3F)CS(=O)(=O)N4)n[nH]2)C1 ; CCC(=O)CNC(=O)OC1CCC(c2cc(Nc3ccc4c(c3F)CS(=O)(=O)N4)n[nH]2)C1]

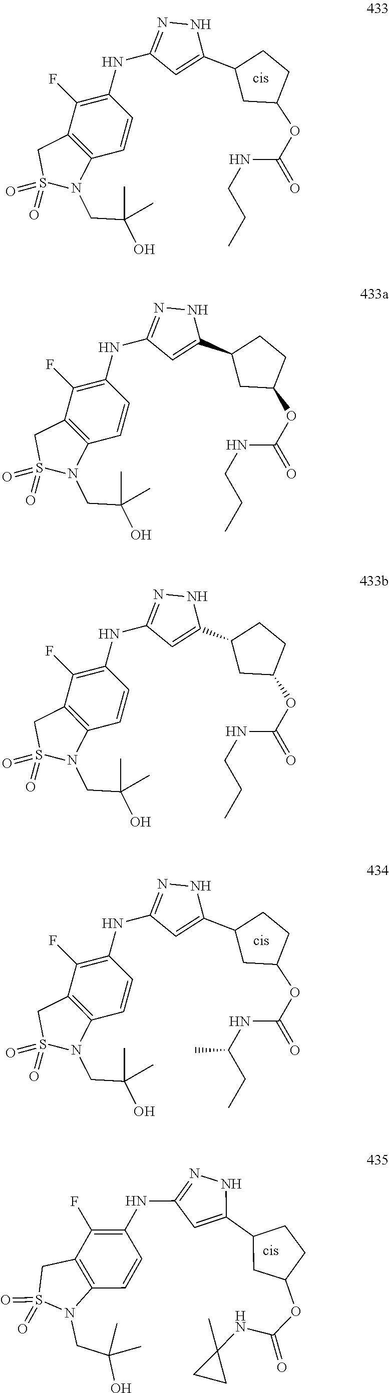 [SMILES: CCCNC(=O)OC1CCC(c2cc(Nc3ccc4c(c3F)CS(=O)(=O)N4CC(C)(C)O)n[nH]2)C1 ; CCCNC(=O)O[C@@H]1CC[C@H](c2cc(Nc3ccc4c(c3F)CS(=O)(=O)N4CC(C)(C)O)n[nH]2)C1 ; CCCNC(=O)O[C@H]1CC[C@@H](c2cc(Nc3ccc4c(c3F)CS(=O)(=O)N4CC(C)(C)O)n[nH]2)C1 ; CC[C@H](C)NC(=O)OC1CCC(c2cc(Nc3ccc4c(c3F)CS(=O)(=O)N4CC(C)(C)O)n[nH]2)C1 ; CC(C)(O)CN1c2ccc(Nc3cc(C4CCC(OC(=O)NC5(C)CC5)C4)[nH]n3)c(F)c2CS1(=O)=O]

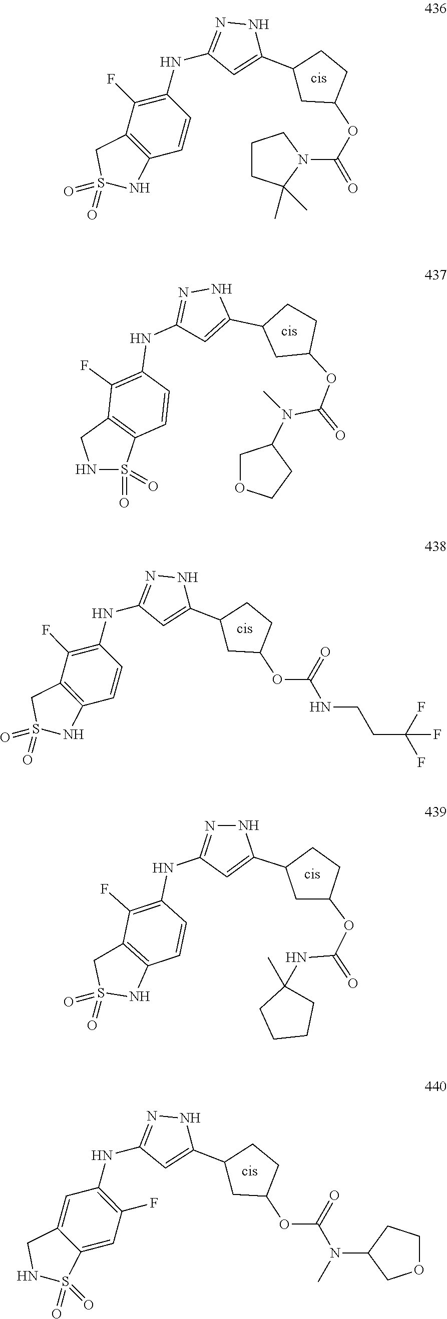 [SMILES: CC1(C)CCCN1C(=O)OC1CCC(c2cc(Nc3ccc4c(c3F)CS(=O)(=O)N4)n[nH]2)C1 ; CN(C(=O)OC1CCC(c2cc(Nc3ccc4c(c3F)CNS4(=O)=O)n[nH]2)C1)C1CCOC1 ; O=C(NCCC(F)(F)F)OC1CCC(c2cc(Nc3ccc4c(c3F)CS(=O)(=O)N4)n[nH]2)C1 ; CC1(NC(=O)OC2CCC(c3cc(Nc4ccc5c(c4F)CS(=O)(=O)N5)n[nH]3)C2)CCCC1 ; CN(C(=O)OC1CCC(c2cc(Nc3cc4c(cc3F)S(=O)(=O)NC4)n[nH]2)C1)C1CCOC1]

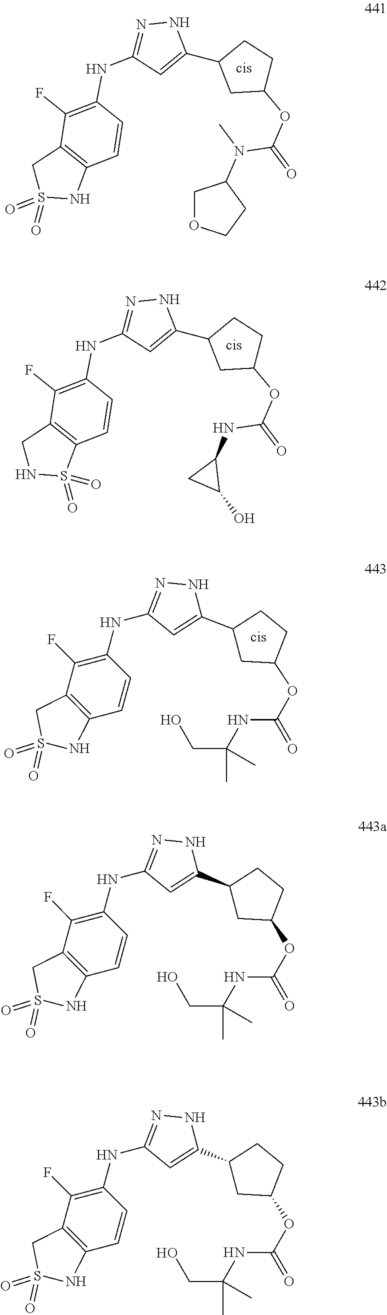 [SMILES: CN(C(=O)OC1CCC(c2cc(Nc3ccc4c(c3F)CS(=O)(=O)N4)n[nH]2)C1)C1CCOC1 ; O=C(N[C@@H]1C[C@H]1O)OC1CCC(c2cc(Nc3ccc4c(c3F)CNS4(=O)=O)n[nH]2)C1 ; CC(C)(CO)NC(=O)OC1CCC(c2cc(Nc3ccc4c(c3F)CS(=O)(=O)N4)n[nH]2)C1 ; CC(C)(CO)NC(=O)O[C@@H]1CC[C@H](c2cc(Nc3ccc4c(c3F)CS(=O)(=O)N4)n[nH]2)C1 ; CC(C)(CO)NC(=O)O[C@H]1CC[C@@H](c2cc(Nc3ccc4c(c3F)CS(=O)(=O)N4)n[nH]2)C1]

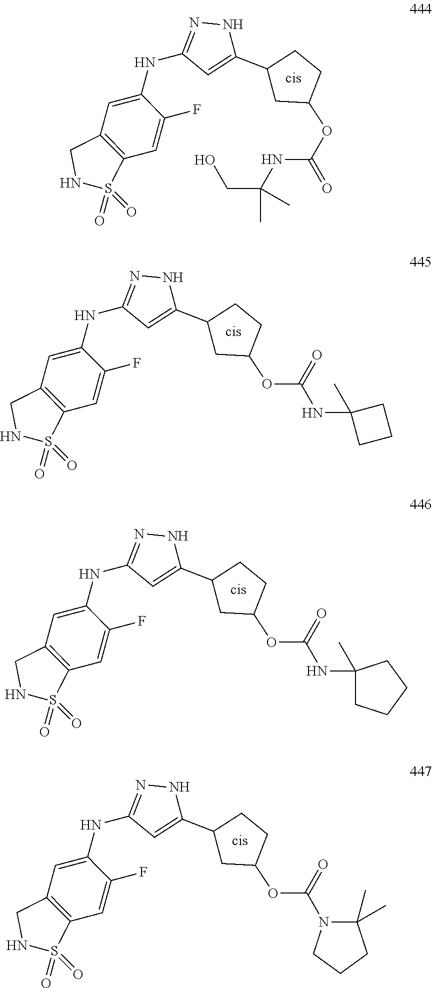 [SMILES: CC(C)(CO)NC(=O)OC1CCC(c2cc(Nc3cc4c(cc3F)S(=O)(=O)NC4)n[nH]2)C1 ; CC1(NC(=O)OC2CCC(c3cc(Nc4cc5c(cc4F)S(=O)(=O)NC5)n[nH]3)C2)CCC1 ; CC1(NC(=O)OC2CCC(c3cc(Nc4cc5c(cc4F)S(=O)(=O)NC5)n[nH]3)C2)CCCC1 ; CC1(C)CCCN1C(=O)OC1CCC(c2cc(Nc3cc4c(cc3F)S(=O)(=O)NC4)n[nH]2)C1]

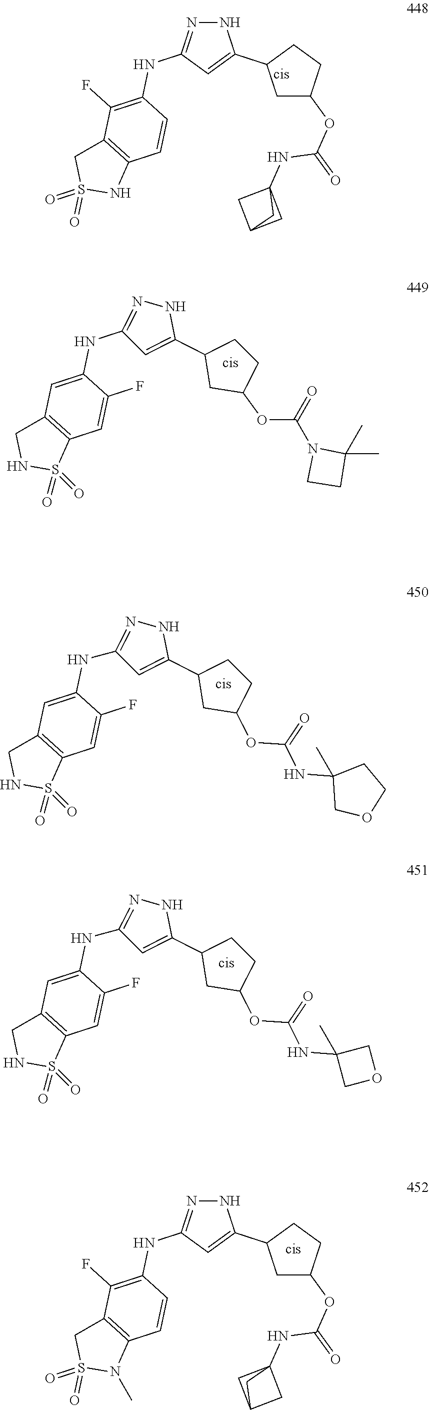 [SMILES: O=C(NC12CC(C1)C2)OC1CCC(c2cc(Nc3ccc4c(c3F)CS(=O)(=O)N4)n[nH]2)C1 ; CC1(C)CCN1C(=O)OC1CCC(c2cc(Nc3cc4c(cc3F)S(=O)(=O)NC4)n[nH]2)C1 ; CC1(NC(=O)OC2CCC(c3cc(Nc4cc5c(cc4F)S(=O)(=O)NC5)n[nH]3)C2)CCOC1 ; CC1(NC(=O)OC2CCC(c3cc(Nc4cc5c(cc4F)S(=O)(=O)NC5)n[nH]3)C2)COC1 ; CN1c2ccc(Nc3cc(C4CCC(OC(=O)NC56CC(C5)C6)C4)[nH]n3)c(F)c2CS1(=O)=O]

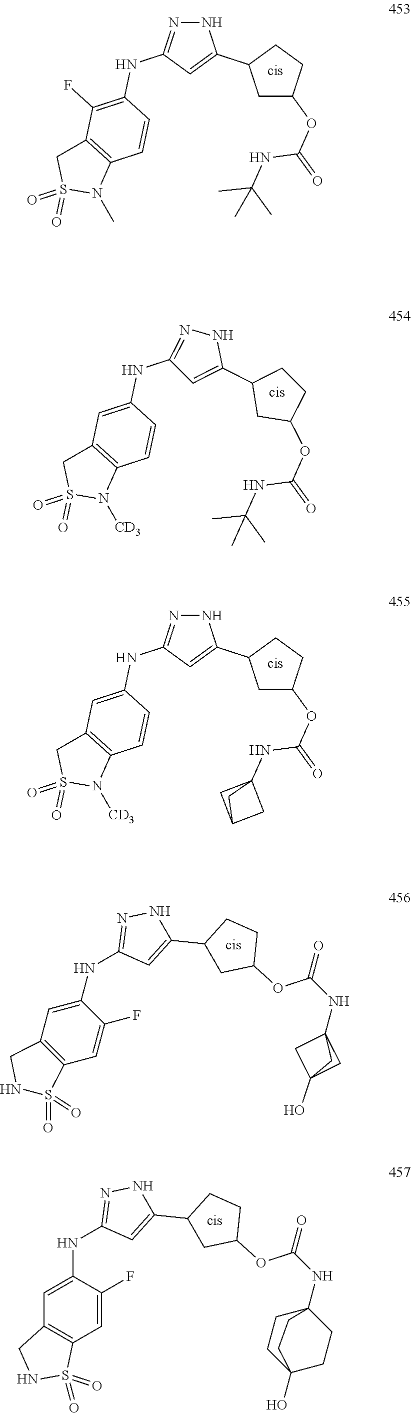 [SMILES: CN1c2ccc(Nc3cc(C4CCC(OC(=O)NC(C)(C)C)C4)[nH]n3)c(F)c2CS1(=O)=O ; [2H]C([2H])([2H])N1c2ccc(Nc3cc(C4CCC(OC(=O)NC(C)(C)C)C4)[nH]n3)cc2CS1(=O)=O ; [2H]C([2H])([2H])N1c2ccc(Nc3cc(C4CCC(OC(=O)NC56CC(C5)C6)C4)[nH]n3)cc2CS1(=O)=O ; O=C(NC12CC(O)(C1)C2)OC1CCC(c2cc(Nc3cc4c(cc3F)S(=O)(=O)NC4)n[nH]2)C1 ; O=C(NC12CCC(O)(CC1)CC2)OC1CCC(c2cc(Nc3cc4c(cc3F)S(=O)(=O)NC4)n[nH]2)C1]

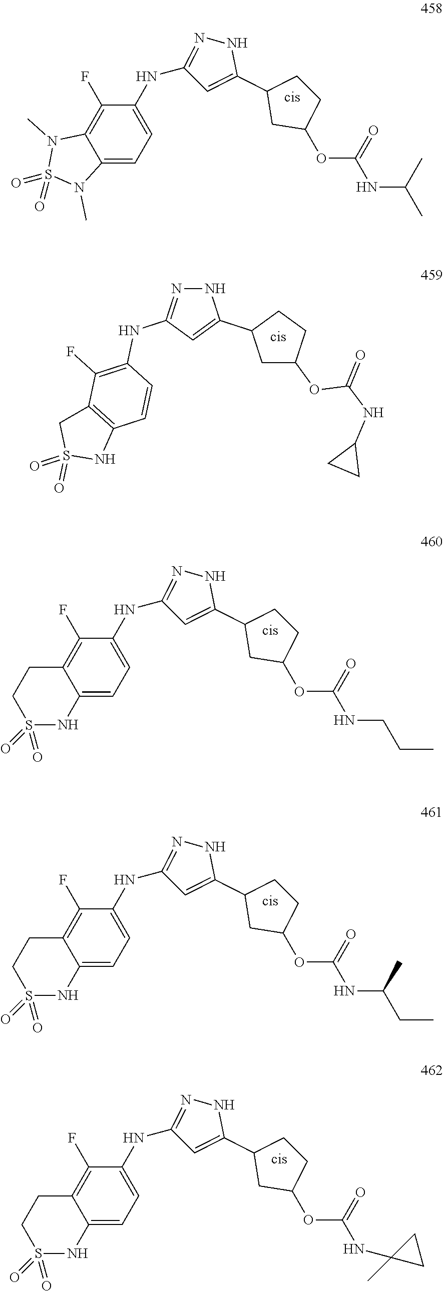 [SMILES: CC(C)NC(=O)OC1CCC(c2cc(Nc3ccc4c(c3F)N(C)S(=O)(=O)N4C)n[nH]2)C1 ; O=C(NC1CC1)OC1CCC(c2cc(Nc3ccc4c(c3F)CS(=O)(=O)N4)n[nH]2)C1 ; CCCNC(=O)OC1CCC(c2cc(Nc3ccc4c(c3F)CCS(=O)(=O)N4)n[nH]2)C1 ; CC[C@H](C)NC(=O)OC1CCC(c2cc(Nc3ccc4c(c3F)CCS(=O)(=O)N4)n[nH]2)C1 ; CC1(NC(=O)OC2CCC(c3cc(Nc4ccc5c(c4F)CCS(=O)(=O)N5)n[nH]3)C2)CC1]

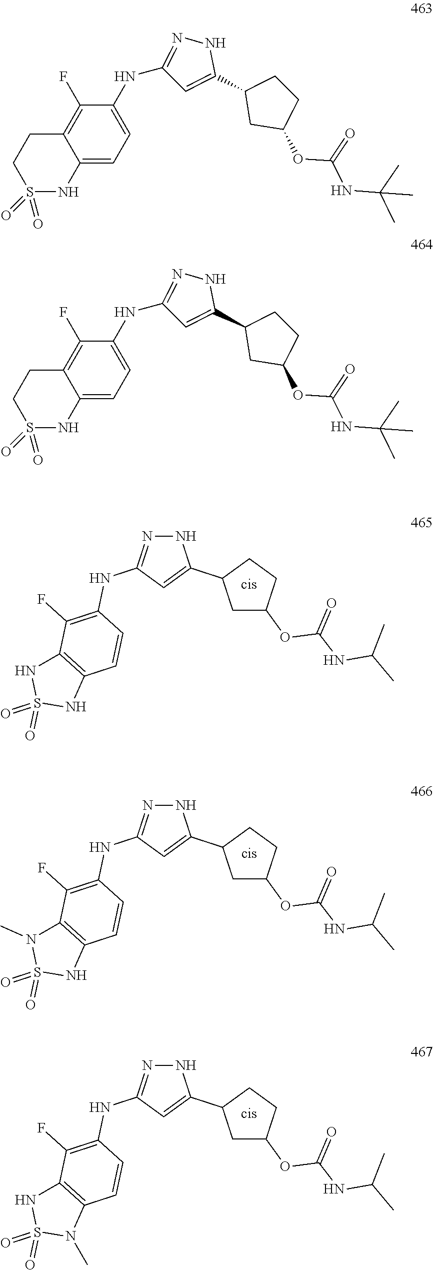

Aspect 20. A pharmaceutical composition comprising a compound of anyone of aspects 1-19, or a pharmaceutically acceptable salt thereof, or a stereoisomer, a tautomer or a prodrug thereof, and at least one pharmaceutically acceptable carrier or excipient.

Aspect 21. A method of treating cancer, comprising administering to a subject in need thereof a compound of anyone of aspects 1-19, or a pharmaceutically acceptable salt, or a stereoisomer, a tautomer or a prodrug thereof.

### DETAILED DESCRIPTION OF THE INVENTION

The following terms have the indicated meanings throughout the specification:

As used herein, including the appended Aspects, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁-6 alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

The term “aryl” used alone or in combination with other terms refers to a group selected from: 5- and 6-membered carbocyclic aromatic rings, e.g., phenyl;
bicyclic ring systems such as 7- to 12-membered bicyclic ring systems, wherein at least one ring is carbocyclic and aromatic, e.g., naphthyl and indanyl; and,
tricyclic ring systems such as 10- to 15-membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “aryl-alkyl-” refers to an alkyl group as defined above which is further substituted by an aryl group. Examples of an aryl-alkyl group include aryl-C₁₋₈alkyl, such as phenylethyl, or phenylmethyl(benzyl).

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur(S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides. The term “C-linked heteroaryl” as used herein means that the heteroaryl group is connected to the core molecule by a bond from a C-atom of the heteroaryl ring

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 1O-ring forming members with 1, 2, 3, or 4 heteroatom ring members independently selected from nitrogen (N), sulfur(S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur(S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur(S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refers to S, SO or SO₂.

“Hydrogen” and “H” are interchangeable and refer to anyone of protium (¹H), deuterium (²H) or tritium (³H). “Deuterated analog” refers to one or more protiums (¹H) of the compound are substituted with equal numbers of deuteriums (²H).

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired product of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

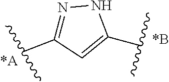 [SMILES: *c1cc(*)[nH]n1]

may undergo tautomerism to form

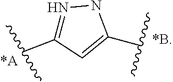 [SMILES: *c1cc(*)[nH]n1]

Wherein *A and *B refer to the position substituents connect to pyrazole.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as the contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be a solution, water solution, oil suspension concentrate, lyophilized powder or the like. Preferably, the formulation of the pharmaceutical composition is selected from a tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### ABBREVIATIONS

| | |
|---|---|
| (Boc)₂O | di-tert-butyl dicarbonate |
| Ac₂O | Acetic anhydride |
| AcOH | Acetic Acid |
| DCM | dichloromethane |
| DIEA | diisopropylethylamine |
| DMAP | 4-dimethylaminopyridine |
| DMF | Dimethylformamide |
| DMSO | Dimethyl sulfoxide |
| EA | Ethyl acetate |
| EA, EtOAc | Ethyl acetate |
| EDC | 1-(3-Dimethylaminopropyl)-3-ethylcarbodiimide |
| | hydrochloride |
| EDCI | N-Ethyl-N′-(3-dimethylaminopropyl)carbodiimide |
| | hydrochloride |
| Eq. | equivalent |
| Et₃N, TEA | triethyl amine |
| EtOH | ethanol |
| Fe | iron |
| HATU | 1-[bis(dimethylamino)methylene]-1H-1,2,3- |
| | triazolo[4,5-b]pyridinium 3-oxid |
| | hexafluorophosphate |
| HCl | Hydrochloric Acid |
| HOBT | 1-Hydroxybenzotriazole |
| MeCN | acetonitrile |
| MeOH | Methanol |
| NaBH₄ | Sodium borohydride |
| NBS | N-Bromosuccinimide |
| Pd(dppf)Cl₂ | [1,1′-Bis(diphenylphosphino)ferrocene] |
| | dichloropalladium(II) |
| Pd(OAc)₂ | Palladium(II)acetate |
| PE | petroleum ether |
| PreP-HPLC | Preparative High-Performance Liquid Chromatography |
| Prep-TCL | Preparative thin-layer chromatography |
| RT or rt | room temperature |
| t-BuONO | tert-Butyl nitrite |
| TFA | trifluoroacetic acid |
| THF | tetrahydrofuran |
| TLC | thin-layer chromatography |
| TMSCN | Trimethylsilyl cyanide |
| Tol or tol | Toluene |
| R_{f} | Retention Factor |
| Cbz | benzyloxycarbonyl |
| XantPhos | 4,5-bis(diphenylphosphino)-9,9-dimethylxanthene |
| Pd₂(dba)₃ | Tris(dibenzylideneacetone)dipalladium |
| BrettPhos | Chloro(2-dicyclohexylphosphino-3,6-dimethoxy-2′,4′,6′- |
| Pd G2 | tri-i-propyl-1,1′-biphenyl)(2′-amino-1,1′-biphenyl- |
| | 2-yl)palladium(II) |
| FA | formic acid |
| BH₃-Me₂S | borane dimethylsulfide |
| MeMgBr | Methyl magnesium bromide |
| pH | Potential Of Hydrogen |
| TBS | tert-butyl dimethylsilyl |
| t-BuOH | tert-butyl alcohol |
| LIHMDS | Lithium Hexamethyldisilazide |
| MtBE | Methyl tert-butyl ether |
| t-BuNH₂ | tert-Butylamine |
| AIBN | 2,2′-Azobis(2-methylpropionitrile) |
| mg | Milligrams |
| mL | Milliliters |
| mmol | Millimole |
| CbzCl | benzyl carbonochloridate |
| PTSA | p-Toluenesulfonamide |
| Brettphos | Methanesulfonato (2-dicyclohexylphosphino-3,6- |
| Pd G3 | dimethoxy-2′,4′,6′-tri-i-propyl-1,1′-biphenyl) |
| | (2′-amino-1,1′-biphenyl-2-yl)palladium(II) |
| MsCl | Methanesulfonyl chloride |
| CMBP | 4-cyano-3-methoxyimino-1-(N-tert-butoxycarbonyl) |
| | pyrrolidine |
| AcCl | Ethanoyl chloride |
| TfOH | Trifluoromethanesulfonic acid |
| DMAP | 4-Dimethylaminopyridine |
| NCS | N-Chlorosuccinimide |
| PhCl | chlorobenzene |
| m-CPBA | m-Chloroperbenzoic Acid |
| ACN | Acetonitrile |

### Examples

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

1H NMR spectra were recorded on a Agilent instrument operating at 400 MHz. ¹HNMR spectra were obtained using CDCl₃, CD₂Cl₂, CD₃OD, D₂O, d₆-DMSO, de-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 μm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 μm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 μm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 μm, Gemini NXC 18) at a flow rate of 20 ml/min, injection volume 2 ml, at room temperature and UV Detection at 214 nm and 254 nm.

### Example 1: cis-3-(3-((5-fluoro-1,1-dioxido-2,3-dihydrobenzo[d]isothiazol-6-yl)amino)-1H-pyrazol-5-yl)cyclopentyl isopropylcarbamate

### Step 1: methyl 1,4-dioxaspiro[4.4]nonane-7-carboxylate

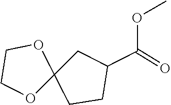

Two parallel reactions were performed. A solution of methyl 3-oxocyclopentanecarboxylate (50.0 g, 351.74 mmol) in toluene (500 mL) was treated with ethylene glycol (43.66 g, 703.47 mmol) and 4-toluenesulfonic acid (6.06 g, 35.17 mmol). The mixture was heated to reflux and stirred for 4 h under nitrogen atmosphere. Each batch was quenched with sat. aq NaHCO₃separately, then the two bathes were combined and concentrated under reduced pressure. The residue was diluted with EA (2 L) and washed with aq NaHCO₃ (500 mL). The aqueous layer was further extracted with EA (500 mL×2). The combined organic layers were washed with brine (500 mL), dried over anhydrous Na₂SO₄, then filtered and concentrated under reduced pressure. The residue was purified by silica gel column chromatography, eluting with PE/EA (1:1; R_{f}=0.43) to afford the product (34 g, 26% yield). ¹H NMR (400 MHz, Chloroform-d) δ=4.00-3.84 (m, 4H), 3.68 (s, 3H), 2.98-2.85 (m, 1H), 2.09 (d, J=8.9 Hz, 2H), 2.06-1.88 (m, 3H), 1.85-1.78 (m, 1H).

### Step 2:3-oxo-3-(1,4-dioxaspiro[4.4]nonan-7-yl)propanenitrile

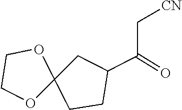 [SMILES: N#CCC(=O)C1CCC2(C1)OCCO2]

Two parallel reactions were performed. To a solution of methyl 1,4-dioxaspiro[4.4]nonane-7-carboxylate (16.0 g, 85.93 mmol) in THF (320 mL), acetonitrile (10.58 g, 257.78 mmol) and sodium hydride (10.31 g, 257.78 mmol, 60%) were added at 0° C. under nitrogen atmosphere. The resulting mixture was heated to reflux and stirred for 4 h. Two batches were combined and poured into sat. aq NH₄Cl (500 mL) at 0° C. and stirred for 30 mins. The layers separated, and the aqueous layer was extracted with EA (500 mL×3). The combined organic layer was washed with brine (200 mL), dried over anhydrous Na₂SO₄, filtered, and concentrated under vacuum. The residue was purified by silica gel chromatography, eluting with PE/EA (10:1; R_{f}=0.29) to afford the product (30.1 g, 90% yield). ¹H NMR (400 MHz, Chloroform-d) δ=3.96-3.86 (m, 4H), 3.54 (d, J=0.7 Hz, 2H), 3.25-3.10 (m, 1H), 2.11-2.00 (m, 3H), 1.98-1.78 (m, 3H).

### Step 3:1-(tert-butyl)-3-(1,4-dioxaspiro[4.4]nonan-7-yl)-1H-pyrazol-5-amine

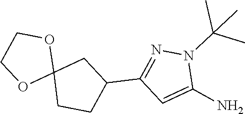 [SMILES: CC(C)(C)n1nc(C2CCC3(C2)OCCO3)cc1N]

To a solution of 3-oxo-3-(1,4-dioxaspiro[4.4]nonan-7-yl)propanenitrile (30.0 g, 153.68 mmol) and tert-butylhydrazine mono hydrochloride (57.45 g, 461.03 mmol) in ethyl alcohol (300 mL) was added triethylamine (46.65 g, 461.03 mmol) at 20° C. The resulting mixture was heated to reflux and stirred for 2 h under nitrogen atmosphere. The reaction mixture was concentrated under reduced pressure, then the residue was purified by silica gel chromatography, eluting with PE/EA (5:1; R_{f}=0.24) to afford the product (27 g, 64% yield). ¹H NMR (400 MHz, DMSO-d₆) δ=5.22 (s, 1H), 4.71 (s, 2H), 3.86-3.76 (m, 4H), 2.89 (tt, J=7.8, 9.9 Hz, 1H), 2.04 (dd, J=8.0, 13.4 Hz, 1H), 1.96-1.82 (m, 2H), 1.78-1.66 (m, 2H), 1.65-1.53 (m, 1H), 1.47 (s, 9H).

### Step 4: benzyl(1-(tert-butyl)-3-(1,4-dioxaspiro[4.4]nonan-7-yl)-1H-pyrazol-5-yl)carbamate

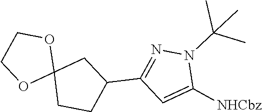 [SMILES: CC(C)(C)n1nc(C2CCC3(C2)OCCO3)cc1NC(=O)OCc1ccccc1]

To a solution of 1-(tert-butyl)-3-(1,4-dioxaspiro[4.4]nonan-7-yl)-1H-pyrazol-5-amine (18 g, 67.83 mmol) in acetone (1 L), benzyl carbonochloridate (23.14 g, 135.67 mmol) was added portion wise at 0° C. The mixture was stirred at room temperature for 2 h, then NaHCO₃ (18.24 g, 217.07 mmol) was added in portions. The mixture was further stirred at this temperature for 26 h. The reaction mixture was filtered and concentrated under reduced pressure to afford the crude product (28.5 g, >99% yield), which was used in the next step without further purification.

### Step 5: benzyl(1-(tert-butyl)-3-(3-oxocyclopentyl)-1H-pyrazol-5-yl)carbamate

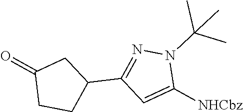 [SMILES: CC(C)(C)n1nc(C2CCC(=O)C2)cc1NC(=O)OCc1ccccc1]

A solution of benzyl(1-(tert-butyl)-3-(1,4-dioxaspiro[4.4]nonan-7-yl)-1H-pyrazol-5-yl)carbamate (27.5 g, 68.84 mmol) in acetone (3 L) and water (300 mL) was treated with 4-toluenesulfonic acid (1.54 g, 8.95 mmol). The mixture was stirred at 60° C. for 4 h under nitrogen atmosphere. The reaction mixture was concentrated under reduced pressure to remove the most of acetone. The aqueous residue was extracted with DCM (500 mL×3). The combined organic phase was washed with brine (500 mL), dried with anhydrous Na₂SO₄, then filtered and concentrated under vacuum. The residue was purified by silica gel chromatography, eluting with PE/EA (10:1) to afford the product (23 g, 87% yield). ¹H NMR (400 MHz, DMSO-d₆) δ=9.12 (br s, 1H), 7.44-7.28 (m, 5H), 6.03 (s, 1H), 5.12 (s, 2H), 3.35-3.32 (m, 1H), 2.48-2.41 (m, 1H), 2.33-2.18 (m, 4H), 1.97-1.87 (m, 1H), 1.48 (s, 9H).

### Step 6: cis-benzyl(1-(tert-butyl)-3-(3-hydroxycyclopentyl)-1H-pyrazol-5-yl)carbamate

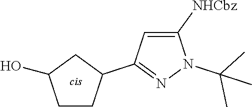 [SMILES: CC(C)(C)n1nc(C2CCC(O)C2)cc1NC(=O)OCc1ccccc1]

A solution of benzyl(1-(tert-butyl)-3-(3-oxocyclopentyl)-1H-pyrazol-5-yl)carbamate (19 g, 53.46 mmol) in THF (200 mL) was cooled to −65° C. A solution of LiBHEt₃ (1 M, 106.9 mL) was added dropwise and the resulting mixture was stirred at −65° C. for 1.5 h under nitrogen atmosphere. The reaction mixture was quenched with sat. aq. NaHCO₃ (50 mL). Water (200 mL) was added, and the mixture was extracted with EA (100 mL×3). The combined organic phase was washed with brine (200 mL), dried over anhydrous Na₂SO₄, then filtered and concentrated under vacuum. The residue was purified by prep-HPLC (column; mobile phase: [water (NH₄HCO₃)-acetone]; B %: 40%-65%, 20 min). Benzyl(1-(tert-butyl)-3-((1S,3R)-3-hydroxycyclopentyl)-1H-pyrazol-5-yl)carbamate (11 g, 57% yield) was obtained. 1H NMR (400 MHz, DMSO-d₆) δ=9.06 (br s, 1H), 7.57-7.12 (m, 5H), 5.92 (s, 1H), 5.75 (s, 1H), 5.12 (s, 2H), 4.57 (d, J=4.4 Hz, 1H), 4.22-4.06 (m, 1H), 2.89 (q, J=8.6 Hz, 1H), 2.25-2.13 (m, 1H), 1.89-1.80 (m, 1H), 1.76-1.66 (m, 2H), 1.60-1.55 (m, 1H), 1.54-1.41 (m, 9H).

### Step 7: cis-benzyl(1-(tert-butyl)-3-(3-(((4-nitrophenoxy) carbonyl)oxy) cyclopentyl)-1H-pyrazol-5-yl)carbamate

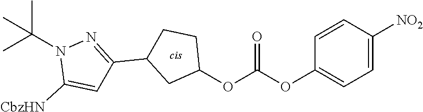 [SMILES: CC(C)(C)n1nc(C2CCC(OC(=O)Oc3ccc([N+](=O)[O-])cc3)C2)cc1NC(=O)OCc1ccccc1]

To a solution of cis-benzyl(1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 8: cis-benzyl(1-(tert-butyl)-3-(3-((isopropylcarbamoyl)oxy) cyclopentyl)-1H-pyrazol-5-yl)carbamate

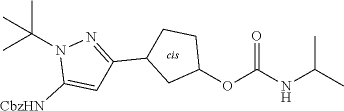 [SMILES: CC(C)NC(=O)OC1CCC(c2cc(NC(=O)OCc3ccccc3)n(C(C)(C)C)n2)C1]

To a solution of cis-benzyl(1-(tert-butyl)-3-(3-(((4-nitrophenoxy) carbonyl)oxy) cyclopentyl)-1H-pyrazol-5-yl)carbamate (11.7 g, 22.4 mmol) in THF (100 mL) was added propan-2-amine (6.6 g, 112.1 mmol). The resulting mixture was stirred at room temperature for 1 h. The resulting mixture was concentrated under reduced pressure, and the residue was purified by silica gel chromatography (PE/EA=1:1) to afford the product (7.7 g, 80% yield). LC-MS (ESI): m/z [M+H]⁺=443.3.

### Step 9: cis-3-(5-amino-1-(tert-butyl)-1H-pyrazol-3-yl)cyclopentyl isopropylcarbamate

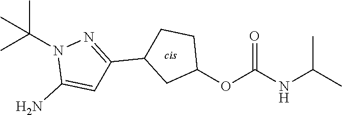 [SMILES: CC(C)NC(=O)OC1CCC(c2cc(N)n(C(C)(C)C)n2)C1]

To a solution of cis-benzyl(1-(tert-butyl)-3-(3-((isopropylcarbamoyl)oxy) cyclopentyl)-1H-pyrazol-5-yl)carbamate (7.5 g, 16.9 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (4.5 g, 84% yield). LC-MS (ESI): m/z [M+H]⁺=309.3.

### Step 10: methyl 4-bromo-2-(chlorosulfonyl)-5-fluorobenzoate

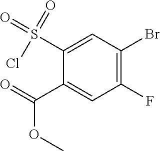 [SMILES: COC(=O)c1cc(F)c(Br)cc1S(=O)(=O)Cl]

To a solution of methyl 2-amino-4-bromo-5-fluorobenzoate (2 g, 8.1 mmol) in HCl (6M, 30 mL) and acetic acid (10 mL), a solution of sodium nitrite (0.6 g, 8.9 mmol) in water (5 ml) was added dropwise at −5° C. The resulting mixture was stirred at −5° C. for 30 mins after addition. Then a solution of sodium bisulfite (12.6 g, 121.4 mmol) in HCl (6 M, 15 mL) was added dropwise followed by CuCl₂ (0.4 g, 4.1 mmol). The resulting suspension was warmed to room temperature and stirred for 3 h. The reaction mixture was poured into ice water (20 ml) and extracted with EA (50 mL×3). The combined organic layers were dried over anhydrous sodium sulfate, filtrated, and concentrated under reduced pressure to afford the crude product (2.5 g, 94% yield, LC-MS (ESI): m/z [M+H]⁺=331.5), which was used in the next step without further purification.

### Step 11:6-bromo-5-fluorobenzo[d]isothiazol-3 (2H)-one 1,1-dioxide

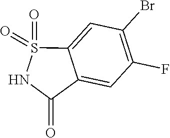 [SMILES: O=C1NS(=O)(=O)c2cc(Br)c(F)cc21]

To a stirred solution of methyl 4-bromo-2-(chlorosulfonyl)-5-fluorobenzoate (2.5 g, 7.5 mmol) in THF (30 mL) was added aqueous ammonia (25%, 4 mL) dropwise. The resulting solution was stirred at room temperature for 2 h. The resulting mixture was concentrated under vacuum, and the residue was triturated with EA and filtered to afford the product (1.9 g, 90% yield). LC-MS (ESI): m/z [M+H]⁺=280.1.

### Step 12:6-bromo-5-fluoro-2,3-dihydrobenzo[d]isothiazole 1,1-dioxide

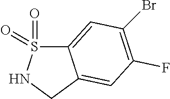 [SMILES: O=S1(=O)NCc2cc(F)c(Br)cc21]

To a stirring solution of 6-bromo-5-fluorobenzo[d]isothiazol-3 (2H)-one 1,1-dioxide (600 mg, 2.15 mmol) in THF (30 mL) was added BH₃-THF (1 M, 10.7 mL, 10.75 mmol) dropwise at 0° C. under nitrogen atmosphere. The resulting solution was heated to reflux and stirred under nitrogen atmosphere for 3 h. The solution was cooled to 0° C., quenched with MeOH (10 mL), and concentrated under reduced pressure. The residue was purified by silica gel column chromatography, eluting with PE/EA (1:1) to afford the product (450 mg, 79% yield). LC-MS (ESI): m/z [M+H]⁺=266.1.

### Step 13: cis-3-(1-(tert-butyl)-5-((5-fluoro-1,1-dioxido-2,3-dihydrobenzo[d]isothiazol-6-yl)amino)-1H-pyrazol-3-yl)cyclopentyl isopropylcarbamate

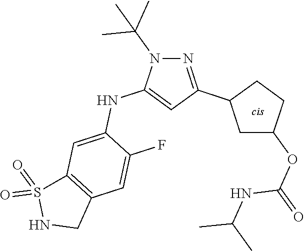 [SMILES: CC(C)NC(=O)OC1CCC(c2cc(Nc3cc4c(cc3F)CNS4(=O)=O)n(C(C)(C)C)n2)C1]

To a mixture of 6-bromo-5-fluoro-2,3-dihydrobenzo[d]isothiazole 1,1-dioxide (100 mg, 0.38 mmol) and cis-3-(5-amino-1-(tert-butyl)-1H-pyrazol-3-yl)cyclopentyl isopropylcarbamate (116.6 mg, 0.38 mmol) in andydrous 1,4-dioxane (10 mL) was added Pd₂(dba)₃ (36.7 mg, 0.04 mmol), XantPhos (44.0 mg, 0.08 mmol) and K₃PO₄ (241.6 mg, 1.1 mmol). The reaction mixture was stirred under nitrogen atmosphere at 90° C. for 16 h. The mixture was filtered, and the filtrate was concentrated under reduced pressure. The residue was purified by silica gel column chromatography, eluting with PE/EA (1:9) to afford the product (115 mg, 62% yield). LC-MS (ESI): m/z [M+H]⁺=494.3.

### Step 14: cis-3-(3-((5-fluoro-1,1-dioxido-2,3-dihydrobenzo[d]isothiazol-6-yl)amino)-1H-pyrazol-5-yl)cyclopentyl isopropylcarbamate

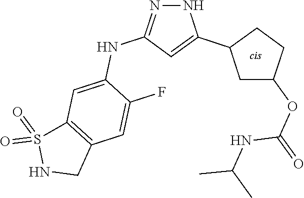 [SMILES: CC(C)NC(=O)OC1CCC(c2cc(Nc3cc4c(cc3F)CNS4(=O)=O)n[nH]2)C1]

A solution of cis-3-(1-(tert-butyl)-3-((5-fluoro-1,1-dioxido-2,3-dihydrobenzo[d]isothiazol-6-yl)amino)-1H-pyrazol-5-yl)cyclopentyl isopropylcarbamate (80 mg, 0.16 mmol) in DCM (10 mL) was treated with triflic acid (0.5 mL) dropwise. The resulting mixture was stirred at room temperature for 2 h. The reaction mixture was quenched and basified to pH=8 with sat. aq NaHCO₃ at 0° C. The layers separated and the aqueous layer was extracted with DCM (100 mL×3). The combined organic layers were washed with brine (50 mL) and dried over anhydrous Na₂SO₄. After filtration, the filtrate was concentrated under reduced pressure. The residue was purified by preparative HPLC (Waters Sunfire C18: RD-CO-058 column, eluting with 28%-48% of acetonitrile (containing 0.1% FA) in water (containing 0.1% FA)) to afford the product (32.3 mg, 46%) in a racemic form, which was further separated by Chiral Prep-HPLC to give:

Enantiomer 1 (Example 1a, 98.6% ee); Retention time: 6.47 min. ¹H NMR (500 MHz, DMSO-d₆) δ 11.91 (s, 1H), 8.66 (d, J=1.9 Hz, 1H), 8.61 (d, J=7.5 Hz, 1H), 7.72 (t, J=4.9 Hz, 1H), 7.34 (d, J=11.4 Hz, 1H), 6.95 (d, J=7.4 Hz, 1H), 5.79 (s, 1H), 5.00 (s, 1H), 4.27 (d, J=4.8 Hz, 2H), 3.58 (m, 1H), 2.48-2.41 (m, 1H), 2.02 (m, 1H), 1.95-1.83 (m, 1H), 1.79-1.43 (m, 3H), 1.03 (d, J=6.3 Hz, 6H).

LC-MS (ESI): m/z [M+H]⁺=438.2.

Enantiomer 2 (Example 1b, 95.6% ee); Retention time: 7.07 min. ¹H NMR (500 MHz, DMSO-d₆) δ 11.91 (s, 1H), 8.66 (d, J=1.9 Hz, 1H), 8.61 (d, J=7.5 Hz, 1H), 7.72 (t, J=4.9 Hz, 1H), 7.34 (d, J=11.4 Hz, 1H), 6.95 (d, J=7.4 Hz, 1H), 5.79 (s, 1H), 5.00 (s, 1H), 4.27 (d, J=4.8 Hz, 2H), 3.58 (m, 1H), 2.48-2.41 (m, 1H), 2.02 (m, 1H), 1.95-1.83 (m, 1H), 1.79-1.43 (m, 3H), 1.03 (d, J=6.3 Hz, 6H).

LC-MS (ESI): m/z [M+H]⁺=438.2.

Chiral analytical method: Column: I-Cellulose-5, 4.6 mm×250 mm, 5 μm; Mobile phase: A for Hexane and B for EtOH (0.1% 2M NH₃ MeOH); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 μm; Mobile phase: A for Hexane and B for EtOH (0.2% 2M NH₃ MeOH); Gradient: Mobile Phase A: Mobile Phase B=60: 40 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 2: cis-3-(3-((1,1-dioxido-2,3-dihydrobenzo[d]isothiazol-5-yl)amino)-1H-pyrazol-5-yl)cyclopentyl isopropylcarbamate

### Step 1:5-bromo-2,3-dihydrobenzo[d]isothiazole 1,1-dioxide

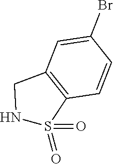 [SMILES: O=S1(=O)NCc2cc(Br)ccc21]

To a stirring solution of 5-bromobenzo[d]isothiazol-3 (2H)-one 1,1-dioxide (750 mg, 2.86 mmol) in THF (30 mL) was added BH₃-Me₂S (4 M, 2.86 mL, 11.44 mmol) dropwise at 0° C. under nitrogen atmosphere. The resulting solution was heated to reflux and stirred under nitrogen atmosphere for 3 h. The resulting mixture was cooled to 0° C., quenched with MeOH (10 mL), and concentrated under reduced pressure. The residue was purified by silica gel column chromatography, eluting with PE/EA (1:1) to afford the product (550 mg, 78%). LC-MS (ESI): m/z [M+H]⁺=248.3.

### Step 2: cis-3-(1-(tert-butyl)-5-((1,1-dioxido-2,3-dihydrobenzo[d]isothiazol-5-yl)amino)-1H-pyrazol-3-yl)cyclopentyl isopropylcarbamate

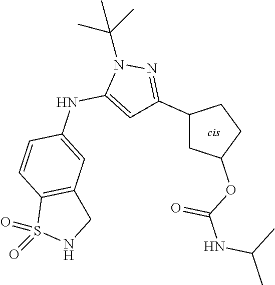 [SMILES: CC(C)NC(=O)OC1CCC(c2cc(Nc3ccc4c(c3)CNS4(=O)=O)n(C(C)(C)C)n2)C1]

To a mixture of 5-bromo-2,3-dihydrobenzo[d]isothiazole 1,1-dioxide (100 mg, 0.40 mmol) and cis-3-(5-amino-1-(tert-butyl)-1H-pyrazol-3-yl)cyclopentyl isopropylcarbamate (123.2 mg, 0.40 mmol) in andydrous 1,4-dioxane (10 mL) was added Pd₂(dba)₃ (36.7 mg, 0.04 mmol), XantPhos (46.2 mg, 0.08 mmol) and K₃PO₄ (254.4 mg, 1.2 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:9) to afford the product (50 mg, 26%). LC-MS (ESI): m/z [M+H]⁺=476.4.

### Step 3: cis-3-(3-((1,1-dioxido-2,3-dihydrobenzo[d]isothiazol-5-yl)amino)-1H-pyrazol-5-yl)cyclopentyl isopropylcarbamate

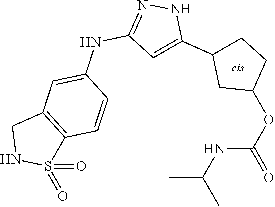 [SMILES: CC(C)NC(=O)OC1CCC(c2cc(Nc3ccc4c(c3)CNS4(=O)=O)n[nH]2)C1]

A solution of cis-3-(1-(tert-butyl)-5-((1,1-dioxido-2,3-dihydrobenzo[d]isothiazol-5-yl)amino)-1H-pyrazol-3-yl)cyclopentyl isopropylcarbamate (50 mg, 0.10 mmol) in formic acid (5 mL) was stirred at 75° C. for 16 h. The solution was concentrated under reduced pressure. The residue was purified by prep HPLC (Waters XSelect C18: RD-CO-094 column, eluting with 27%-52% of acetonitrile (containing 0.1% FA) in water (containing 0.1% FA)) to afford the product (12 mg, 27%). ¹H NMR (500 MHz, DMSO-d₆) δ 11.90 (s, 1H), 9.00 (s, 1H), 7.53 (d, J=8.5 Hz, 1H), 7.50-7.38 (m, 2H), 7.30 (d, J=8.5 Hz, 1H), 6.95 (d, J=6.9 Hz, 1H), 5.70 (s, 1H), 5.06-4.93 (m, 1H), 4.29 (d, J=4.9 Hz, 2H), 3.65-3.50 (m, 1H), 3.15-2.95 (m, 1H), 2.39-2.28 (m, 1H), 2.08-1.98 (m, 1H), 1.96-1.84 (m, 1H), 1.81-1.66 (m, 2H), 1.65-1.54 (m, 1H), 1.03 (d, J=6.0 Hz, 6H). LC-MS (ESI): m/z [M+H]⁺=420.5

### Example 3: cis-3-(3-((1,1-dioxido-2,3-dihydrobenzo[d]isothiazol-6-yl)amino)-1H-pyrazol-5-yl)cyclopentyl isopropylcarbamate

### Step 1:6-bromo-2,3-dihydrobenzo[d]isothiazole 1,1-dioxide

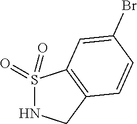 [SMILES: O=S1(=O)NCc2ccc(Br)cc21]

To a stirring solution of 6-bromobenzo[d]isothiazol-3 (2H)-one 1,1-dioxide (600 mg, 2.29 mmol) in THF (20 mL) was added BH₃-Me₂S (4 M, 2.29 mL, 9.16 mmol) dropwise at 0° C. under nitrogen atmosphere. The resulting solution was heated to reflux and stirred under nitrogen atmosphere for 3 h. The resulting mixture was cooled to 0° C., quenched with MeOH (10 mL), and concentrated under reduced pressure. The residue was purified by silica gel column chromatography, eluting with PE/EA (1:1) to afford the product (350 mg, 62%). LC-MS (ESI): m/z [M+H]⁺=248.3.

### Step 2: cis-3-(1-(tert-butyl)-5-((1,1-dioxido-2,3-dihydrobenzo[d]isothiazol-6-yl)amino)-1H-pyrazol-3-yl)cyclopentyl isopropylcarbamate

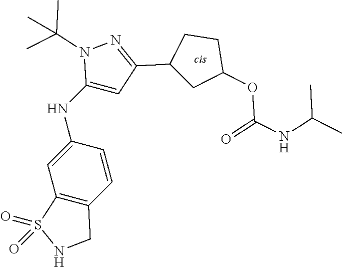 [SMILES: CC(C)NC(=O)OC1CCC(c2cc(Nc3ccc4c(c3)S(=O)(=O)NC4)n(C(C)(C)C)n2)C1]

To a mixture of 6-bromo-2,3-dihydrobenzo[d]isothiazole 1,1-dioxide (100 mg, 0.40 mmol) and cis-3-(5-amino-1-(tert-butyl)-1H-pyrazol-3-yl)cyclopentyl isopropylcarbamate (123.2 mg, 0.40 mmol) in andydrous 1,4-dioxane (10 mL) was added Pd₂(dba)₃ (36.7 mg, 0.04 mmol), XantPhos (46.2 mg, 0.08 mmol) and K₃PO₄ (254.4 mg, 1.2 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:9) to afford the product (20 mg, 10%). LC-MS (ESI): m/z [M+H]⁺=476.4.

### Step 3: cis-3-(3-((1,1-dioxido-2,3-dihydrobenzo[d]isothiazol-6-yl)amino)-1H-pyrazol-5-yl)cyclopentyl isopropylcarbamate

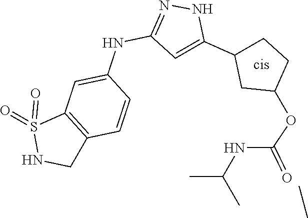

A solution of cis-3-(1-(tert-butyl)-5-((1,1-dioxido-2,3-dihydrobenzo[d]isothiazol-6-yl)amino)-1H-pyrazol-3-yl)cyclopentyl isopropylcarbamate (20 mg, 0.04 mmol) in formic acid (5 mL) was stirred at 75° C. for 16 h. The solution was concentrated under reduced pressure. The residue was purified by prep HPLC (Waters SunFire C18: RD-CO-095 column, eluting with 25%-55% of acetonitrile (containing 0.1% FA) in water (containing 0.1% FA)) to afford the product (10 mg, 57%). ¹H NMR (500 MHz, DMSO-d₆) δ 11.83 (brs, 1H), 8.84 (s, 1H), 7.97 (s, 1H), 7.62 (s, 1H), 7.38 (dd, J=8.5, 1.6 Hz, 1H), 7.30 (d, J=8.5 Hz, 1H), 6.93 (d, J=6.4 Hz, 1H), 5.63 (s, 1H), 5.06-4.93 (m, 1H), 4.30-4.20 (m, 2H), 3.52-3.40 (m 1H), 3.13-2.99 (m, 1H), 2.45 (dd, J=13.8, 7.2 Hz, 1H), 2.07-1.97 (m, 1H), 1.96-1.85 (m, 1H), 1.8-1.66 (m, 2H), 1.65-1.55 (m, 1H), 1.03 (d, J=6.3 Hz, 6H). LC-MS (ESI): m/z [M+H]⁺=420.3

### Example 4: cis-3-(5-((3,3-dimethyl-1,1-dioxido-2,3-dihydrobenzo[d]isothiazol-6-yl)amino)-1H-pyrazol-3-yl)cyclopentyl isopropylcarbamate

### Step 1:6-bromo-3-chlorobenzo[d]isothiazole 1,1-dioxide

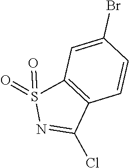 [SMILES: O=S1(=O)N=C(Cl)c2ccc(Br)cc21]

A mixture of 6-bromobenzo[d]isothiazol-3 (2H)-one 1,1-dioxide (1.5 g, 5.72 mmol) in POCl₃ (20 mL) was heated to reflux and stirred under nitrogen atmosphere for 16 h. The mixture was concentrated under reduced pressure, and the residue was slurried with PE/EA (8:1, 20 mL) and filtered to afford the crude product (1.2 g, 75%, LC-MS (ESI): m/z [M-Cl+MeOH]⁺=276.3), which was used in the next step without further purification.

### Step 2:6-bromo-3,3-dimethyl-2,3-dihydrobenzo[d]isothiazole 1,1-dioxide

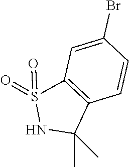

To a stirring mixture of 6-bromo-3-chlorobenzo[d]isothiazole 1,1-dioxide (1.2 g, 4.28 mmol) and LiCl (0.72 g, 17.12 mmol) in THF (30 mL) was added MeMgBr (1 M, 34.24 mL, 34.24 mmol) dropwise at −5° C. under nitrogen atmosphere. The solution was warmed to room temperature and stirred under nitrogen atmosphere for 3 h. The solution was cooled to 0° C. and quenched with sat. aq NH₄Cl (10 mL). The resulting mixture was extracted with EA (100 mL×3). The combined organic phases were washed with brine, dried over anhydrous Na₂SO₄, and concentrated under reduced pressure. The residue was purified by silica gel column chromatography, eluting with PE/EA (1:1) to afford the product (0.5 g, 43%). LC-MS (ESI): m/z [M+H]⁺=276.3.

### Step 3: cis-3-(1-(tert-butyl)-5-((3,3-dimethyl-1,1-dioxido-2,3-dihydrobenzo[d]isothiazol-6-yl)amino)-1H-pyrazol-3-yl)cyclopentyl isopropylcarbamate

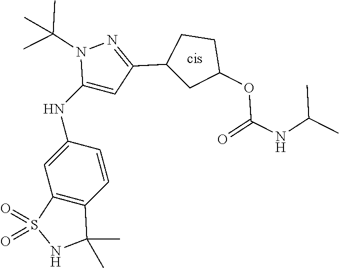 [SMILES: CC(C)NC(=O)OC1CCC(c2cc(Nc3ccc4c(c3)S(=O)(=O)NC4(C)C)n(C(C)(C)C)n2)C1]

To a mixture of 6-bromo-3,3-dimethyl-2,3-dihydrobenzo[d]isothiazole 1,1-dioxide (100 mg, 0.362 mmol) and cis-3-(5-amino-1-(tert-butyl)-1H-pyrazol-3-yl)cyclopentyl isopropylcarbamate (111.6 mg, 0.362 mmol) in andydrous 1,4-dioxane (10 mL) was added Pd₂(dba)₃ (33 mg, 0.036 mmol), XantPhos (42 mg, 0.072 mmol) and K₃PO₄ (230 mg, 1.086 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:9) to afford the product (120 mg, 66%). LC-MS (ESI): m/z [M+H]⁺=504.5.

### Step 4: cis-3-(3-((3,3-dimethyl-1,1-dioxido-2,3-dihydrobenzo[d]isothiazol-6-yl)amino)-1H-pyrazol-5-yl)cyclopentyl isopropylcarbamate

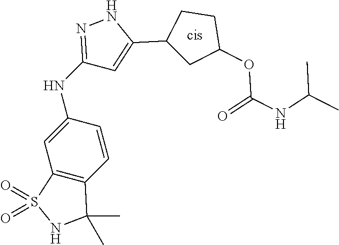 [SMILES: CC(C)NC(=O)OC1CCC(c2cc(Nc3ccc4c(c3)S(=O)(=O)NC4(C)C)n[nH]2)C1]

To a stirred solution of cis-3-(1-(tert-butyl)-5-((3,3-dimethyl-1,1-dioxido-2,3-dihydrobenzo[d]isothiazol-6-yl)amino)-1H-pyrazol-3-yl)cyclopentyl isopropylcarbamate (120 mg, 0.238 mmol) in DCM (5 mL) was added triflic acid (0.5 mL) dropwise. The solution was stirred at room temperature for 3 h. The solution was diluted with DCM (50 mL) and basified to pH=8 with sat. aq NaHCO₃ at 0° C. The layers separated and the aqueous layer was extracted with DCM (100 mL×3). The combined organic layers were dried over anhydrous Na₂SO₄, filtered, and concentrated under reduced pressure. The residue was purified by prep HPLC (Waters SunFire C18: RD-CO-058 column, eluting with 34%-49% of acetonitrile (containing 0.1% FA) in water (containing 0.1% FA) to afford the product (85 mg, 80%). ¹H NMR (500 MHz, DMSO-d₆) δ 11.83 (s, 1H), 8.83 (s, 1H), 7.90 (s, 1H), 7.80-7.6 (m, 1H), 7.52-7.25 (m, 2H), 6.96 (s, 1H), 5.62 (s, 1H), 5.15-4.90 (m, 1H), 3.70-3.50 (m, 1H), 3.19-3.93 (m, 1H), 2.47-2.40 (m, 1H), 2.19-1.83 (m, 2H), 1.82-1.57 (m, 3H), 1.55-1.36 (m, 6H), 1.03 (d, J=5.6 Hz, 6H). LC-MS (ESI): m/z [M+H]⁺=448.3.

### Example 5: cis-3-(5-((3-methyl-1,1-dioxido-2,3-dihydrobenzo[d]isothiazol-6-yl)amino)-1H-pyrazol-3-yl)cyclopentyl isopropylcarbamate

### Step 1:6-bromo-3-methylbenzo[d]isothiazole 1,1-dioxide

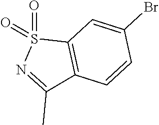

To a stirring mixture of 6-bromo-3-chlorobenzo[d]isothiazole 1,1-dioxide (1.2 g, 4.28 mmol) and LiCl (0.72 g, 17.12 mmol) in THF (30 mL) was added MeMgBr (1 M, 34.24 mL, 34.24 mmol) dropwise at −5° C. under nitrogen atmosphere. The solution was stirred at room temperature for 3 h. The solution was cooled down to 0° C. and quenched with sat. aq NH₄Cl (10 mL). The resulting mixture was extracted with EA (100 mL×3), and the combined organic phases were washed with brine and concentrated under reduced pressure. The residue was purified by silica gel column chromatography, eluting with PE/EA (1:1) to afford the product (0.3 g, 27%). LC-MS (ESI): m/z [M+H]⁺=260.3.

### Step 2:6-bromo-3-methyl-2,3-dihydrobenzo[d]isothiazole 1,1-dioxide

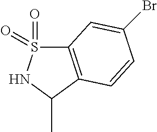

To a stirring solution of 6-bromo-3-methylbenzo[d]isothiazole 1,1-dioxide (0.3 g, 1.15 mmol) in MeOH (20 mL) was added NaBH₄ (131 mg, 3.45 mmol) at 0° C. under nitrogen atmosphere. The mixture was stirred at room temperature for 3 h. The mixture was concentrated under reduced pressure. The residue was purified by silica gel column chromatography, eluting with PE/EA (1:1) to afford the product (0.2 g, 66%). LC-MS (ESI): m/z [M+H]⁺=262.3.

### Step 3: cis-3-(1-(tert-butyl)-5-((3-methyl-1,1-dioxido-2,3-dihydrobenzo[d]isothiazol-6-yl)amino)-1H-pyrazol-3-yl)cyclopentyl isopropylcarbamate

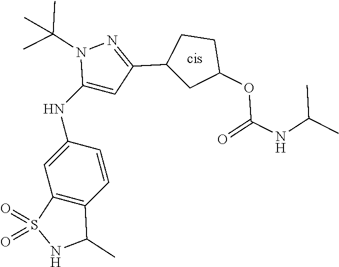 [SMILES: CC(C)NC(=O)OC1CCC(c2cc(Nc3ccc4c(c3)S(=O)(=O)NC4C)n(C(C)(C)C)n2)C1]

To a mixture of 6-bromo-3-methyl-2,3-dihydrobenzo[d]isothiazole 1,1-dioxide (200 mg, 0.763 mmol), cis-3-(5-amino-1-(tert-butyl)-1H-pyrazol-3-yl)cyclopentyl isopropylcarbamate (235 mg, 0.763 mmol) in andydrous 1,4-dioxane (20 mL) was added Pd₂(dba)₃ (70 mg, 0.076 mmol), XantPhos (88 mg, 0.153 mmol) and K₃PO₄ (485 mg, 2.289 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:9) to afford the product (120 mg, 32%). LC-MS (ESI): m/z [M+H]⁺=490.5

### Step 4: cis-3-(5-((3-methyl-1,1-dioxido-2,3-dihydrobenzo[d]isothiazol-6-yl)amino)-1H-pyrazol-3-yl)cyclopentyl isopropylcarbamate

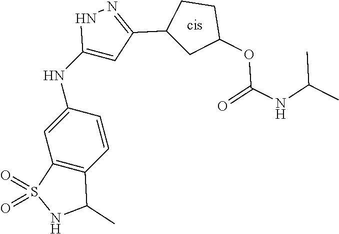 [SMILES: CC(C)NC(=O)OC1CCC(c2cc(Nc3ccc4c(c3)S(=O)(=O)NC4C)[nH]n2)C1]

To a stirred solution of cis-3-(1-(tert-butyl)-5-((3-methyl-1,1-dioxido-2,3-dihydrobenzo[d]isothiazol-6-yl)amino)-1H-pyrazol-3-yl)cyclopentyl isopropylcarbamate (120 mg, 0.245 mmol) in DCM (5 mL) was added triflic acid (0.5 mL). The solution was stirred at room temperature for 3 h. The solution was diluted with DCM (50 mL), basified to pH=8 with sat. aq NaHCO₃ and extracted with DCM (50 mL×2). The combined organic layers were dried over anhydrous Na₂SO₄, filtered, and concentrated under reduced pressure. The residue was purified by preparative HPLC (Waters SunFire C18: RD-CO-095 column, eluting with 28%-53% of acetonitrile (containing 0.1% FA) in water (containing 0.1% FA) to afford the product (90 mg, 85%) in a racemic form, which was further separated by Chiral Prep-HPLC to give:

Enantiomer 1 (Example 5a, 100% ee); Retention time: 10.46 min. ¹H NMR (500 MHz, DMSO-d₆) δ 11.83 (s, 1H), 8.86 (s, 1H), 7.95 (d, J=1.4 Hz, 1H), 7.72 (d, J=4.2 Hz, 1H), 7.39 (dd, J=8.5, 1.9 Hz, 1H), 7.34 (d, J=8.5 Hz, 1H), 6.95 (d, J=7.5 Hz, 1H), 5.62 (d, J=1.9 Hz, 1H), 5.00 (s, 1H), 4.60-4.52 (m, 1H), 3.65-3.52 (m, 1H), 3.13-3.00 (m, 1H), 2.48-2.41 (m, 1H), 2.08-1.98 (m, 1H), 1.95-1.85 (m, 1H), 1.80-1.66 (m, 2H), 1.64-1.55 (m, 1H), 1.39 (d, J=6.6 Hz, 3H), 1.03 (d, J=6.2 Hz, 6H).

LC-MS (ESI): m/z [M+H]⁺=434.3.

Enantiomer 2 (Example 5b, 95.4% ee); Retention time: 12.06 min. ¹H NMR (500 MHz, DMSO-d₆) δ 11.83 (s, 1H), 8.86 (s, 1H), 7.95 (s, 1H), 7.72 (d, J=4.3 Hz, 1H), 7.38 (d, J=8.4 Hz, 1H), 7.34 (d, J=8.5 Hz, 1H), 6.95 (d, J=7.9 Hz, 1H), 5.62 (s, 1H), 5.05-4.95 (m, 1H), 4.60-4.52 (m, 1H), 3.62-3.55 (m, 1H), 3.11-3.02 (m, 1H), 2.48-2.41 (m, 1H), 2.06-1.97 (m, 1H), 1.94-1.85 (m, 1H), 1.77-1.67 (m, 2H), 1.64-1.55 (m, 1H), 1.39 (d, J=6.6 Hz, 3H), 1.03 (d, J=6.3 Hz, 6H). LC-MS (ESI): m/z [M+H]⁺=434.4.

Enantiomer 3 (Example 5c, 100% ee); Retention time: 13.10 min. ¹H NMR (500 MHz, DMSO-d₆) δ 11.83 (s, 1H), 8.85 (s, 1H), 7.95 (s, 1H), 7.72 (d, J=4.3 Hz, 1H), 7.38 (d, J=7.8 Hz, 1H), 7.34 (d, J=8.5 Hz, 1H), 6.95 (d, J=7.3 Hz, 1H), 5.62 (s, 1H), 5. 06-4.93 (m, 1H), 4.63-4.49 (m, 1H), 3.64-3.52 (m, 1H), 3.11-3.00 (m, 1H), 2.49-2.42 (m, 1H), 2.08-1.97 (m, 1H), 1.96-1.85 (m, 1H), 1.78-1.67 (m, 2H), 1.65-1.56 (m, 1H), 1.39 (d, J=6.6 Hz, 3H), 1.03 (d, J=6.2 Hz, 6H). LC-MS (ESI): m/z [M+H]⁺=434.1.

Enantiomer 4 (Example 5d, 99.1% ee); Retention time: 15.48 min. ¹H NMR (500 MHz, DMSO-d₆) δ 11.83 (s, 1H), 8.86 (s, 1H), 7.95 (d, J=1.3 Hz, 1H), 7.72 (d, J=4.3 Hz, 1H), 7.38 (dd, J=8.5, 1.8 Hz, 1H), 7.34 (d, J=8.5 Hz, 1H), 6.95 (d, J=7.7 Hz, 1H), 5.62 (d, J=1.8 Hz, 1H), 5.08-4.95 (m, 1H), 4.62-4.51 (m, 1H), 3.65-3.52 (m, 1H), 3.13-2.99 (m, 1H), 2.48-2.40 (m, 1H), 2.07-1.98 (m, 1H), 1.95-1.85 (m, 1H), 1.80-1.67 (m, 2H), 1.65-1.55 (m, 1H), 1.39 (d, J=6.6 Hz, 3H), 1.04 (d, J=6.2 Hz, 6H). LC-MS (ESI): m/z [M+H]⁺=434.3.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 6: cis-3-(3-((3,3-dimethyl-1,1-dioxido-2,3-dihydrobenzo[d]isothiazol-5-yl)amino)-1H-pyrazol-5-yl)cyclopentyl isopropylcarbamate

### Step 1:5-bromo-3-chlorobenzo[d]isothiazole 1,1-dioxide

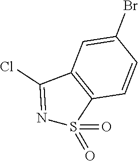 [SMILES: O=S1(=O)N=C(Cl)c2cc(Br)ccc21]

A mixture of 5-bromobenzo[d]isothiazol-3 (2H)-one 1,1-dioxide (500 mg, 1.91 mmol) in POCl₃ (15 mL) was heated to reflux and stirred under nitrogen atmosphere for 16 h. The mixture was concentrated under reduced pressure. The residue was slurried with PE/EA (8:1, 9 mL) and filtered to afford the crude product (450 mg, 84%, LC-MS (ESI): m/z [M-Cl+MeOH]⁺=276.3), which was used in the next step without further purification.

### Step 2:5-bromo-3,3-dimethyl-2,3-dihydrobenzo[d]isothiazole 1,1-dioxide

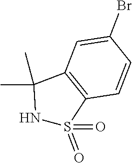

To a stirring mixture of 5-bromo-3-chlorobenzo[d]isothiazole 1,1-dioxide (450 mg, 1.61 mmol) and LiCl (270 mg, 6.44 mmol) in THF (20 mL) was added MeMgBr (1 M, 19.3 mL, 19.32 mmol) dropwise at −5° C. under nitrogen atmosphere. The resulting solution was stirred at room temperature for 16 h. The mixture was cooled to 0° C. and quenched with sat. aq NH₄Cl (10 mL). The resulting mixture was extracted with EA (100 mL×3). The combined organic phases were washed with brine, dried over anhydrous Na₂SO₄, filtered, and concentrated under reduced pressure. The residue was purified by silica gel column chromatography, eluting with PE/EA (1:1) to afford the product (0.5 g, 43%). LC-MS (ESI): m/z [M+H]⁺=276.3.

### Step 3: cis-3-(1-(tert-butyl)-5-((3,3-dimethyl-1,1-dioxido-2,3-dihydrobenzo[d]isothiazol-5-yl)amino)-1H-pyrazol-3-yl)cyclopentyl isopropylcarbamate

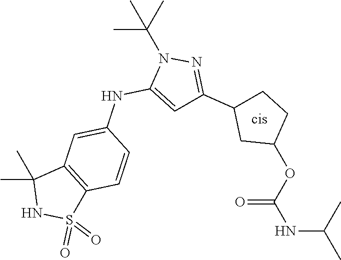 [SMILES: CC(C)NC(=O)OC1CCC(c2cc(Nc3ccc4c(c3)C(C)(C)NS4(=O)=O)n(C(C)(C)C)n2)C1]

To a mixture of 5-bromo-3,3-dimethyl-2,3-dihydrobenzo[d]isothiazole 1,1-dioxide (100 mg, 0.362 mmol) and cis-3-(5-amino-1-(tert-butyl)-1H-pyrazol-3-yl)cyclopentyl isopropylcarbamate (111.6 mg, 0.36 mmol) in andydrous 1,4-dioxane (10 mL) was added Pd₂(dba)₃ (33 mg, 0.04 mmol), XantPhos (42 mg, 0.07 mmol) and K₃PO₄ (230 mg, 1.09 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:1) to afford the product (80 mg, 44%). LC-MS (ESI): m/z [M+H]⁺=504.4.

### Step 4: cis-3-(3-((3,3-dimethyl-1,1-dioxido-2,3-dihydrobenzo[d]isothiazol-5-yl)amino)-1H-pyrazol-5-yl)cyclopentyl isopropylcarbamate

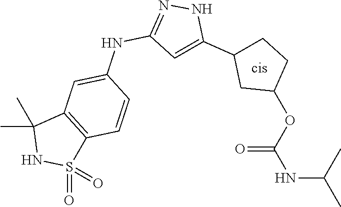 [SMILES: CC(C)NC(=O)OC1CCC(c2cc(Nc3ccc4c(c3)C(C)(C)NS4(=O)=O)n[nH]2)C1]

To a stirred solution of cis-3-(1-(tert-butyl)-5-((3,3-dimethyl-1,1-dioxido-2,3-dihydrobenzo[d]isothiazol-5-yl)amino)-1H-pyrazol-3-yl)cyclopentyl isopropylcarbamate (80 mg, 0.159 mmol) in DCM (5 mL) was added triflic acid (0.5 mL) dropwise. The solution was stirred at room temperature for 3 h. The mixture was diluted with DCM (50 mL) and basified to pH=8 with sat. aq NaHCO₃ and extracted with DCM (50 mL×2). The combined organic layers were dried over anhydrous Na₂SO₄, filtered, and concentrated under reduced pressure. The residue was purified by prep HPLC (Waters SunFire C18: RD-CO-095 column, eluting with 34%-49% of acetonitrile (containing 0.1% FA) in water (containing 0.1% FA) to afford the product (25 mg, 35%). ¹H NMR (500 MHz, DMSO-d₆) δ 11.96 (s, 1H), 9.01 (s, 1H), 7.54 (d, J=11.4 Hz, 2H), 7.48 (d, J=8.6 Hz, 1H), 7.31 (d, J=7.9 Hz, 1H), 6.94 (d, J=7.5 Hz, 1H), 5.68 (s, 1H), 5.06-4.95 (m, 1H), 3.63-3.56 (m, 1H), 3.13-2.98 (m, 1H), 2.47-2.42 (m, 1H), 2.07-1.97 (m, 1H), 1.96-1.85 (m, 1H), 1.79-1.66 (m, 2H), 1.65-1.56 (m, 1H), 1.47 (s, 6H), 1.03 (d, J=6.2 Hz, 6H). LC-MS (ESI): m/z [M+H]⁺=448.4.

### Example 7: cis-3-(3-((2,2-dioxido-1,3-dihydrobenzo[c]isothiazol-5-yl)amino)-1H-pyrazol-5-yl)cyclopentyl isopropylcarbamate and (1S,3R)-3-(3-((2,2-dioxido-1,3-dihydrobenzo[c]isothiazol-5-yl)amino)-1H-pyrazol-5-yl)cyclopentyl isopropylcarbamate

### Step 1: cis-benzyl(1-(tert-butyl)-3-(3-((tert-butyldimethylsilyl)oxy) cyclopentyl)-1H-pyrazol-5-yl)carbamate

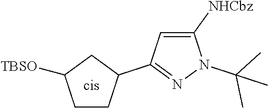 [SMILES: CC(C)(C)n1nc(C2CCC(O[Si](C)(C)C(C)(C)C)C2)cc1NC(=O)OCc1ccccc1]

To a solution of cis-benzyl(1-(tert-butyl)-3-(3-hydroxycyclopentyl)-1H-pyrazol-5-yl)carbamate (200 mg, 5.6 mmol) in DMF (20 mL) was added tert-butyldimethylsilyl chloride (109.6 mg, 7.3 mmol, 0.89 mL, 1.3 eq) and imidazole (57.1 mg, 8.39 mmol, 1.5 eq). The mixture was stirred at 25° C. for 1 hr. The mixture was extracted with ethyl acetate (50 mL×3). The combined organic phase was washed with brine (50 mL×3), dried over anhydrous Na₂SO₄ and then filtered. The filtration was concentrated in vacuum to give a residue. The product was used to next step without further purification. The title compound (200 mg, 4.24 mmol, 76% yield) was obtained. ¹H NMR (400 MHz, Chloroform-d) δ 7.35 (br s, 5H), 6.74-6.38 (m, 1H), 6.08 (br s, 1H), 5.19-5.14 (m, 2H), 4.32-4.22 (m, 1H), 2.97 (br s, 1H), 2.33-2.22 (m, 1H), 1.94 (dd, J=7.63, 3.88 Hz, 1H), 1.87-1.75 (m, 2H), 1.69-1.60 (m, 2H), 1.57-1.54 (m, 9H), 0.88-0.85 (m, 9H), 0.05-0.01 (m, 6H).

### Step 2:1-(tert-butyl)-3-(cis-3-((tert-butyldimethylsilyl)oxy) cyclopentyl)-1H-pyrazol-5-amine

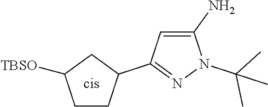 [SMILES: CC(C)(C)n1nc(C2CCC(O[Si](C)(C)C(C)(C)C)C2)cc1N]

To a solution of cis-benzyl(1-(tert-butyl)-3-(3-((tert-butyldimethylsilyl)oxy) cyclopentyl)-1H-pyrazol-5-yl)carbamate (200 mg, 4.2 mmol) in THF (50 mL) was added Pd/C (200 mg, 10% purity, wet). The mixture was stirred at 25° C. under H₂ atmosphere (15 psi) for 2 h. The reaction mixture was filtered over Celite, washed with MeOH, and the filtrate was concentrated under reduced pressure to give a residue. The title compound (100 mg, 70% yield) was obtained. ¹H NMR (400 MHz, Chloroform-d) δ 5.48 (s, 1H), 4.29 (dd, J=6.17, 4.71 Hz, 1H), 3.49 (br s, 2H), 2.95 (t, J=8.80 Hz, 1H), 2.36-2.24 (m, 1H), 2.01-1.90 (m, 1H), 1.87-1.77 (m, 2H), 1.62 (s, 6H), 0.86-0.93 (m, 14H), 0.06 (d, J=1.71 Hz, 6H)

### Step 3: cis-5-((1-(tert-butyl)-3-(3-((tert-butyldimethylsilyl)oxy) cyclopentyl)-1H-pyrazol-5-yl)amino)-1,3-dihydrobenzo[c]isothiazole 2,2-dioxide

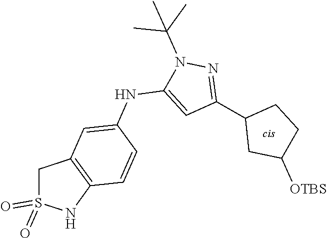 [SMILES: CC(C)(C)n1nc(C2CCC(O[Si](C)(C)C(C)(C)C)C2)cc1Nc1ccc2c(c1)CS(=O)(=O)N2]

To a mixture of 5-bromo-1,3-dihydrobenzo[c]isothiazole 2,2-dioxide (200 mg, 0.81 mmol) and 1-(tert-butyl)-3-(cis-3-((tert-butyldimethylsilyl)oxy) cyclopentyl)-1H-pyrazol-5-amine (289.8 mg, 0.81 mmol) in andydrous t-BuOH (10 mL) was added BrettPhos Pd G2 (74.4 mg, 0.08 mmol) and LiHMDS (1 M, 3.24 ml, 3.24 mmol). The reaction mixture was heated to reflux and stirred under nitrogen atmosphere for 3 h. The mixture was concentrated under reduced pressure. The residue was purified by silica gel column chromatography, eluting with PE/EA (1:9) to afford the product (200 mg, 58%). LC-MS (ESI): m/z [M+H]⁺=505.3.

### Step 4: cis-5-((1-(tert-butyl)-3-(3-hydroxycyclopentyl)-1H-pyrazol-5-yl)amino)-1,3-dihydrobenzo[c]isothiazole 2,2-dioxide

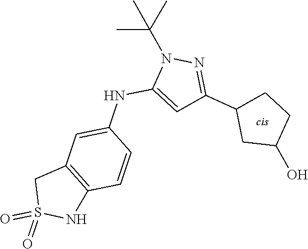 [SMILES: CC(C)(C)n1nc(C2CCC(O)C2)cc1Nc1ccc2c(c1)CS(=O)(=O)N2]

A solution of cis-5-((1-(tert-butyl)-3-(3-((tert-butyldimethylsilyl)oxy) cyclopentyl)-1H-pyrazol-5-yl)amino)-1,3-dihydrobenzo[c]isothiazole 2,2-dioxide (200 mg, 0.39 mmol) in formic acid (5 mL) was stirred at room temperature for 2 h. The solution was concentrated under reduced pressure. The residue was purified by silica gel column chromatography, eluting with PE/EA (1:2) to afford the product (135 mg, 85%). LC-MS (ESI): m/z [M+H]⁺=391.2.

### Step 5: cis-4-nitrophenyl 5-((1-(tert-butyl)-3-(3-(((4-nitrophenoxy) carbonyl)oxy) cyclopentyl)-1H-pyrazol-5-yl)amino)benzo[c]isothiazole-1 (3H)-carboxylate 2,2-dioxide

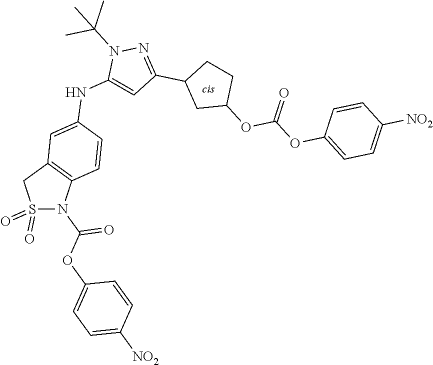 [SMILES: CC(C)(C)n1nc(C2CCC(OC(=O)Oc3ccc([N+](=O)[O-])cc3)C2)cc1Nc1ccc2c(c1)CS(=O)(=O)N2C(=O)Oc1ccc([N+](=O)[O-])cc1]

To a solution of cis-5-((1-(tert-butyl)-3-(3-hydroxycyclopentyl)-1H-pyrazol-5-yl)amino)-1,3-dihydrobenzo[c]isothiazole 2,2-dioxide (130 mg, 0.33 mmol) in DCM (20 mL) was added pyridine (52.5 mg, 0.66 mmol), DMAP (2 mg, 0.02 mmol), 4-nitrophenyl carbonochloridate (132 mg, 0.66 mmol), successively. The resulting mixture was stirred at room temperature for 16 h. The reaction mixture was concentrated under reduced pressure, and the residue was purified by silica gel chromatography, eluting with PE/EA (1:1) to afford the product (148 mg, 61%). LC-MS (ESI): m/z [M+H]⁺=721.2.

### Step 6: cis-3-(1-(tert-butyl)-5-((1-(isopropylcarbamoyl)-2,2-dioxido-1,3-dihydrobenzo[c]isothiazol-5-yl)amino)-1H-pyrazol-3-yl)cyclopentyl isopropylcarbamate

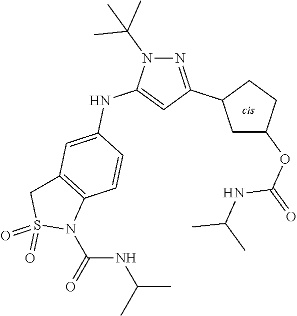 [SMILES: CC(C)NC(=O)OC1CCC(c2cc(Nc3ccc4c(c3)CS(=O)(=O)N4C(=O)NC(C)C)n(C(C)(C)C)n2)C1]

To a stirred solution of cis-4-nitrophenyl 5-((1-(tert-butyl)-3-(3-(((4-nitrophenoxy) carbonyl)oxy) cyclopentyl)-1H-pyrazol-5-yl)amino)benzo[c]isothiazole-1 (3H)-carboxylate 2,2-dioxide (148 mg, 0.27 mmol) in THF (10 mL) was added propan-2-amine (79 mg, 1.33 mmol). The resulting mixture was stirred at room temperature for 1 h. The resulting mixture was concentrated under reduced pressure, and the residue was purified by silica gel column chromatography, eluting with PE/EA (1:9) to afford the product (50 mg, 40%). LC-MS (ESI): m/z [M+H]⁺=561.3.

### Step 7: cis-3-(3-((2,2-dioxido-1,3-dihydrobenzo[c]isothiazol-5-yl)amino)-1H-pyrazol-5-yl)cyclopentyl isopropylcarbamate

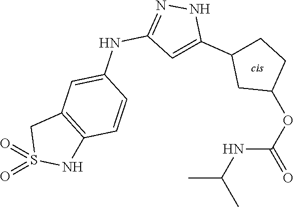 [SMILES: CC(C)NC(=O)OC1CCC(c2cc(Nc3ccc4c(c3)CS(=O)(=O)N4)n[nH]2)C1]

A solution of cis-3-(1-(tert-butyl)-5-((1-(isopropylcarbamoyl)-2,2-dioxido-1,3-dihydrobenzo[c]isothiazol-5-yl)amino)-1H-pyrazol-3-yl)cyclopentyl isopropylcarbamate (34 mg, 0.06 mmol) in DCM (5 mL) was added triflic acid (0.2 mL) dropwise. The solution was stirred at room temperature for 2 h. The solution was diluted with DCM (30 mL) and basified to pH=8 with sat. aq NaHCO₃ and extracted with DCM (30 mL×2). The combined organic layers were dried over anhydrous Na₂SO₄, filtered, and concentrated under reduced pressure. The residue was purified by prep HPLC (Waters SunFire C18: RD-CO-058 column, eluting with 34%-49% of acetonitrile (containing 0.1% FA) in water (containing 0.1% FA) to afford the product in racemic form, which was further purified with chiral PreP-HPLC to give:

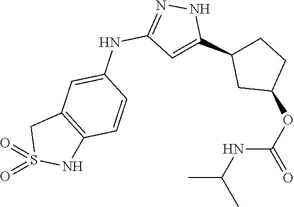 [SMILES: CC(C)NC(=O)O[C@@H]1CC[C@H](c2cc(Nc3ccc4c(c3)CS(=O)(=O)N4)n[nH]2)C1]

Enantiomer 1 ((1R,3S)-3-(3-((2,2-dioxido-1,3-dihydrobenzo[c]isothiazol-5-yl)amino)-1H-pyrazol-5-yl)cyclopentyl isopropylcarbamate, Example 7a, 100% ee); Retention time: 5.02 min. ¹H NMR (500 MHz, DMSO-d₆) δ 11.63 (s, 1H), 9.75 (s, 1H), 8.19 (s, 1H), 7.37 (s, 1H), 7.16 (d, J=7.8 Hz, 1H), 6.95 (d, J=6.8 Hz, 1H), 6.71 (d, J=8.6 Hz, 1H), 5.58 (s, 1H), 4.99 (s, 1H), 4.44 (s, 2H), 3.62-3.58 (m, 1H), 3.11-2.94 (m, 1H), 2.50-2.44 (m, 1H), 2.05-1.97 (m, 1H), 1.95-1.84 (m, 1H), 1.70-1.65 (m, 2H), 1.65-1.59 (m, 1H), 1.03 (d, J=6.2 Hz, 6H). LC-MS (ESI): m/z [M+H]⁺=420.2.

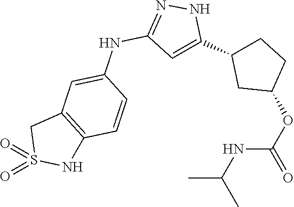 [SMILES: CC(C)NC(=O)O[C@H]1CC[C@@H](c2cc(Nc3ccc4c(c3)CS(=O)(=O)N4)n[nH]2)C1]

Enantiomer 2 ((1S,3R)-3-(3-((2,2-dioxido-1,3-dihydrobenzo[c]isothiazol-5-yl)amino)-1H-pyrazol-5-yl)cyclopentyl isopropylcarbamate, Example 7b, 100% ee); Retention time: 7.89 min. ¹H NMR (500 MHz, DMSO-d₆) δ 11.63 (s, 1H), 9.75 (s, 1H), 8.19 (s, 1H), 7.37 (s, 1H), 7.16 (d, J=7.8 Hz, 1H), 6.95 (d, J=6.8 Hz, 1H), 6.71 (d, J=8.6 Hz, 1H), 5.58 (s, 1H), 4.99 (s, 1H), 4.44 (s, 2H), 3.62-3.58 (m, 1H), 3.11-2.94 (m, 1H), 2.50-2.44 (m, 1H), 2.05-1.97 (m, 1H), 1.95-1.84 (m, 1H), 1.70-1.65 (m, 2H), 1.65-1.59 (m, 1H), 1.03 (d, J=6.2 Hz, 6H). LC-MS (ESI): m/z [M+H]⁺=420.2

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for Hexane and B for EtOH (0.2% 2M NH₃ MeOH); Gradient: Mobile Phase A: Mobile Phase B=20: 80 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 8: cis-3-(3-((5-fluoro-3,3-dimethyl-1,1-dioxido-2,3-dihydrobenzo[d]isothiazol-6-yl)amino)-1H-pyrazol-5-yl)cyclopentyl isopropylcarbamate

### Step 1:6-bromo-3-chloro-5-fluorobenzo[d]isothiazole 1,1-dioxide

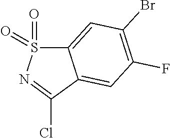 [SMILES: O=S1(=O)N=C(Cl)c2cc(F)c(Br)cc21]

A mixture of 6-bromo-5-fluorobenzo[d]isothiazol-3 (2H)-one 1,1-dioxide (1 g, 3.58 mmol) in POCl₃ (20 mL) was heated to reflux and stirred under nitrogen atmosphere for 16 h. The mixture was concentrated under reduced pressure. The residue was slurried with PE/EA (8:1, 20 mL) and filtered to afford the crude product (800 m g, 80%, LC-MS (ESI): m/z [M-Cl+MeOH]⁺=293.9), which was used in the next step without further purification.

### Step 2:6-bromo-5-fluoro-3,3-dimethyl-2,3-dihydrobenzo[d]isothiazole 1,1-dioxide

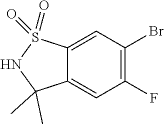

To a stirring mixture of 6-bromo-3-chloro-5-fluorobenzo[d]isothiazole 1,1-dioxide (500 mg, 1.68 mmol) in THF (30 mL) was added MeMgBr (1 M, 16.83 mL, 16.83 mmol) dropwise at −5° C. under nitrogen atmosphere. The solution was stirred at −5° C. for 3 h. The solution was cooled down to 0° C. and quenched with sat. aq. NH₄Cl (10 mL). The resulting mixture was extracted with EA (100 mL×3). The combined organic phases were washed with brine, dried over anhydrous Na₂SO₄, filtered, and concentrated under reduced pressure. The residue was purified by silica gel column chromatography, eluting with PE/EA (1:1) to afford the product (0.3 g, 63%). LC-MS (ESI): m/z [M+H]⁺=293.9.

### Step 3: cis-3-(1-(tert-butyl)-3-((5-fluoro-3,3-dimethyl-1,1-dioxido-2,3-dihydrobenzo[d]isothiazol-6-yl)amino)-1H-pyrazol-5-yl)cyclopentyl isopropylcarbamate

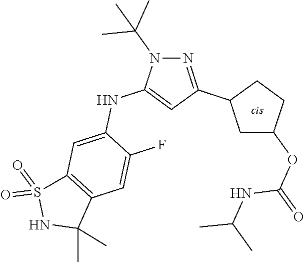 [SMILES: CC(C)NC(=O)OC1CCC(c2cc(Nc3cc4c(cc3F)C(C)(C)NS4(=O)=O)n(C(C)(C)C)n2)C1]

To a mixture of 6-bromo-5-fluoro-3,3-dimethyl-2,3-dihydrobenzo[d]isothiazole 1,1-dioxide (100 mg, 0.342 mmol) and cis-3-(5-amino-1-(tert-butyl)-1H-pyrazol-3-yl)cyclopentyl isopropylcarbamate (105.8 mg, 0.342 mmol) in andydrous 1,4-dioxane (10 mL) was added Pd₂(dba)₃ (32 mg, 0.034 mmol), XantPhos (39 mg, 0.068 mmol) and K₃PO₄ (217.5 mg, 1.026 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:9) to afford the product (120 mg, 66%). LC-MS (ESI): m/z [M+H]⁺=522.2.

### Step 4: cis-3-(3-((5-fluoro-3,3-dimethyl-1,1-dioxido-2,3-dihydrobenzo[d]isothiazol-6-yl)amino)-1H-pyrazol-5-yl)cyclopentyl isopropylcarbamate

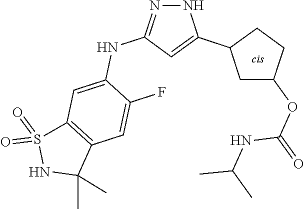 [SMILES: CC(C)NC(=O)OC1CCC(c2cc(Nc3cc4c(cc3F)C(C)(C)NS4(=O)=O)n[nH]2)C1]

To a stirred solution of cis-3-(1-(tert-butyl)-3-((5-fluoro-3,3-dimethyl-1,1-dioxido-2,3-dihydrobenzo[d]isothiazol-6-yl)amino)-1H-pyrazol-5-yl)cyclopentyl isopropylcarbamate (80 mg, 0.153 mmol) in DCM (5 mL) was added triflic acid (0.5 mL) dropwise. The solution was stirred at room temperature for 3 h. The solution was diluted with DCM (50 mL) and basified to pH=8 with sat. aq. NaHCO₃ and extracted with DCM (50 mL×2). The combined organic layers were dried over anhydrous Na₂SO₄, filtered, and concentrated under reduced pressure. The residue was purified by prep HPLC (Waters Sunfire C18: RD-CO-058 column, eluting with 40%-55% of acetonitrile (containing 0.1% FA) in water (containing 0.1% FA) to afford the product (37.6 mg, 53%). ¹H NMR (500 MHz, DMSO-d₆) δ 11.90 (s, 1H), 8.64 (s, 1H), 8.52 (d, J=7.6 Hz, 1H), 7.79 (s, 1H), 7.52 (d, J=11.6 Hz, 1H), 6.95 (d, J=7.3 Hz, 1H), 5.79 (s, 1H), 5.00 (s, 1H), 3.58 (dd, J=13.1, 6.5 Hz, 1H), 3.12-3.00 (m, 1H), 2.48-2.38 (m, 1H), 2.08-1.97 (m, 1H), 1.96-1.84 (m, 1H), 1.70 (dd, J=20.1, 10.4 Hz, 2H), 1.60 (d, J=8.2 Hz, 1H), 1.48 (s, 6H), 1.03 (d, J=6.3 Hz, 6H). LC-MS (ESI): m/z [M+H]⁺=466.2.

### Example 9: cis-3-(3-((4-fluoro-1,1-dioxido-2,3-dihydrobenzo[d]isothiazol-5-yl)amino)-1H-pyrazol-5-yl)cyclopentyl isopropylcarbamate

### Step 1: methyl 6-amino-3-bromo-2-fluorobenzoate

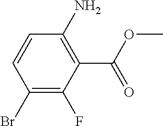

To a solution of 6-amino-3-bromo-2-fluorobenzoic acid (1.0 g, 4.27 mmol) in methanol (20 mL) was added SOCl₂ (2.5 g, 21.3 mmol) dropwise. The reaction mixture was stirred under refluxed for 16 hours. The resulting mixture was cooled to room temperature, concentrated, neutralized with sat. aq NaHCO₃ and extracted with EA. The combined organic layers were washed with brine, dried over Na₂SO₄, and concentrated under vacuum. The residue was purified by silica gel column chromatography, eluting with PE/EA (5:1) to afford the product (346 mg, 33%). LC-MS (ESI): m/z [M+H]⁺=248.0.

### Step 2: methyl 3-bromo-6-(chlorosulfonyl)-2-fluorobenzoate

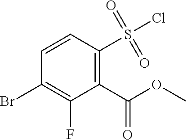 [SMILES: COC(=O)c1c(S(=O)(=O)Cl)ccc(Br)c1F]

To a suspension of methyl 6-amino-3-bromo-2-fluorobenzoate (3.0 g, 12.1 mmol) in hydrochloric acid (12 M, 60 mL), a solution of sodium nitrite (835 mg, 12.1 mmol) in 5 mL water was added dropwise at 0° C. The resulting mixture was kept below 5° C. and stirred for 30 mins after addition. To the reaction mixture CuCl₂ (813 mg, 6.05 mmol), sodium bisulfite (18.9 g, 181.5 mmol) and hydrochloric acid solution (5.5 M, 20 mL) were added sequentially. The reaction mixture was warmed to room temperature and stirred for 3 h. The resulting mixture was extracted with EA (50 mL×3). The combined organic layers were washed with brine, dried over Na₂SO₄, and filtered. The filtrated was concentrated and dried over in vacuum to afford the crude product (3.2 g), which was used in the next step without further purification.

### Step 3:5-bromo-4-fluorobenzo[d]isothiazol-3 (2H)-one 1,1-dioxide

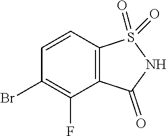 [SMILES: O=C1NS(=O)(=O)c2ccc(Br)c(F)c21]

To a solution of methyl 3-bromo-6-(chlorosulfonyl)-2-fluorobenzoate (3.2 g crude) in THF (50 mL) was added aqueous ammonia (25%, 10 mL) dropwise. The reaction mixture was stirred at room temperature for 16 h. The resulting mixture was concentrated under vacuum, and the residue was triturated with EA and filtered to afford the crude product (3.0 g, LC-MS (ESI): m/z [M+H]⁺=279.9), which was used in the next step without further purification.

### Step 4:5-bromo-4-fluoro-2,3-dihydrobenzo[d]isothiazole 1,1-dioxide

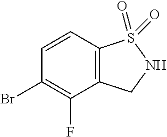 [SMILES: O=S1(=O)NCc2c1ccc(Br)c2F]

To a stirring solution of 5-bromo-4-fluorobenzo[d]isothiazol-3 (2H)-one 1,1-dioxide (210 mg, 0.75 mmol) in THF (5 mL) was added BH₃-Me₂S (2 M, 1.5 mL, 3.0 mmol) dropwise at 0° C. under nitrogen atmosphere. The solution was heated to reflux and stirred for 3 h. The resulting mixture was cooled to 0° C., quenched with MeOH (1.0 mL), and concentrated under reduced pressure. The residue was purified by silica gel column chromatography, eluting with DCM/MeOH (5:1) to afford the product (184 mg, 93%). LC-MS (ESI): m/z [M+H]⁺=265.8

### Step 5: cis-3-(1-(tert-butyl)-3-((4-fluoro-1,1-dioxido-2,3-dihydrobenzo[d]isothiazol-5-yl)amino)-1H-pyrazol-5-yl)cyclopentyl isopropylcarbamate

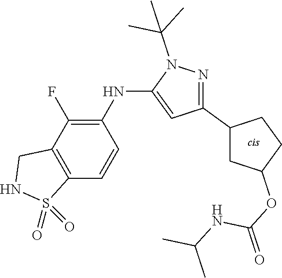 [SMILES: CC(C)NC(=O)OC1CCC(c2cc(Nc3ccc4c(c3F)CNS4(=O)=O)n(C(C)(C)C)n2)C1]

To a mixture of 5-bromo-4-fluoro-2,3-dihydrobenzo[d]isothiazole 1,1-dioxide (100 mg, 0.377 mmol) and cis-3-(5-amino-1-(tert-butyl)-1H-pyrazol-3-yl)cyclopentyl isopropylcarbamate (116 mg, 0.377 mmol) in andydrous 1,4-dioxane (10 mL) was added Pd₂(dba)₃ (17 mg, 0.02 mmol), XantPhos (22 mg, 0.04 mmol) and K₃PO₄ (240 mg, 1.13 mmol). The reaction mixture was stirred at 90° C. under nitrogen atmosphere for 16 h. The mixture was filtered, and the filtrate was concentrated under reduced pressure. The residue was purified by silica gel column chromatography, eluting with DCM/MeOH (20:1) to afford the product (26 mg, 14%). LC-MS (ESI): m/z [M+H]⁺=494.3.

### Step 6: cis-3-(3-((4-fluoro-1,1-dioxido-2,3-dihydrobenzo[d]isothiazol-5-yl)amino)-1H-pyrazol-5-yl)cyclopentyl isopropylcarbamate

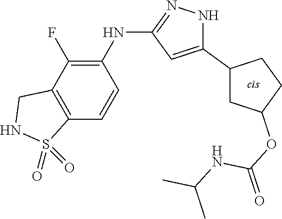 [SMILES: CC(C)NC(=O)OC1CCC(c2cc(Nc3ccc4c(c3F)CNS4(=O)=O)n[nH]2)C1]

To a solution of cis-3-(1-(tert-butyl)-3-((4-fluoro-1,1-dioxido-2,3-dihydrobenzo[d]isothiazol-5-yl)amino)-1H-pyrazol-5-yl)cyclopentyl isopropylcarbamate (26 mg, 0.053 mmol) in DCM (1.5 mL) was added trifluoromethanesulfonic acid (3 drops). The reaction mixture was stirred at room temperature for 2 h. The resulting mixture was neutralized with sat. aq Na₂CO₃ and extracted with DCM (5 mL×3). The combined organic layers were washed with brine, dried over NaSO₄, filtered, and concentrated under reduce pressure. The residue was purified by prep-HPLC (Waters SunFire C18: 19×150 mm, 5 μm, eluting with 33%-45% of acetonitrile (containing 0.1% FA) in water (containing 0.1% FA)) to afford the product in racemic form, which was further separated by Chiral Prep-HPLC to give:

Enantiomer 1 (Example 9a, 100% ee); Retention time: 6.472 min. ¹H NMR (500 MHz, DMSO-d₆) δ=11.99 (s, 1H), 8.72 (s, 1H), 8.25 (s, 1H), 7.70 (t, J=5.0, 1H), 7.48 (d, J=8.6, 1H), 6.95 (d, J=7.3, 1H), 5.83 (s, 1H), 5.00 (s, 1H), 4.41 (d, J=5.1, 2H), 3.58 (m, 1H), 3.13-2.99 (m, 1H), 2.45 (m, 1H), 2.03 (m, 1H), 1.91 (m, 1H), 1.83-1.64 (m, 2H), 1.60 (m, 1H), 1.11-0.93 (m, 6H). LC-MS (ESI): m/z [M+H]⁺=438.4.

Enantiomer 2 (Example 9b, 100% ee); Retention time: 14.841 min. ¹H NMR (500 MHz, DMSO-d₆) δ=11.98 (s, 1H), 8.72 (s, 1H), 8.26 (s, 1H), 7.70 (t, J=5.1, 1H), 7.48 (d, J=8.5, 1H), 6.95 (d, J=7.2, 1H), 5.83 (s, 1H), 5.00 (s, 1H), 4.41 (d, J=5.2, 2H), 3.60-3.55 (m, 1H), 3.10-3.03 (m, 1H), 2.47-2.43 (m, 1H), 2.07-1.98 (m, 1H), 1.98-1.85 (m, 1H), 1.81-1.64 (m, 2H), 1.60 (m, 1H), 1.03 (m, 6H). LC-MS (ESI): m/z [M+H]⁺=438.3;

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=50: 50 (v/v); HPLC Equipment: HPLC-Agilent; Column temperature: 35° C.

Chiral Prep-HPLC Condition: CHIRALPAK IE 21.2 mm×250 mm, 5 μm; Mobile phase: A for MtBE and B for DCM:MeOH=50:50 (v/v) (0.2% 2 M NH₃. MeOH); Gradient: Mobile Phase A: Mobile Phase B=50: 50 (v/v); Flow Rate: 18 mL/min, Wavelength: UV 280 nm and 300 nm, Prep-HPLC Equipment: Prep-HPLC-Gilson; Column temperature: 25° C.

### Example 10: cis-3-(3-((4-methoxy-1,1-dioxido-2,3-dihydrobenzo[d]isothiazol-5-yl)amino)-1H-pyrazol-5-yl)cyclopentyl isopropylcarbamate

### Step 1:5-bromo-4-methoxybenzo[d]isothiazol-3 (2H)-one 1,1-dioxide

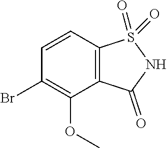

To a solution of 5-bromo-4-fluorobenzo[d]isothiazol-3 (2H)-one 1,1-dioxide (1.0 g, 3.58 mmol) in MeOH (5 mL) was added a solution of sodium methoxide in MeOH (5.4 M, 3.3 mL, 17.92 mmol). The reaction mixture was stirred at 40° C. for 1 h. The resulting mixture was quenched with water (30 mL), adjust pH=4 with 1 N HCl, and extracted with EA (50 mL×3). The combined organic layers were washed with brine, dried over Na₂SO₄, and filtered. The filtrate was concentrated under reduce pressure and dried over in vacuo to afford the crude product (740 mg, 71%, LC-MS (ESI): m/z [M+H]⁺=291.9), which was used in the next step without further purification.

### Step 2:5-bromo-4-methoxy-2,3-dihydrobenzo[d]isothiazole 1,1-dioxide

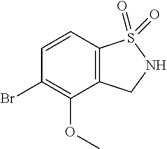

To a stirring solution of 5-bromo-4-methoxybenzo[d]isothiazol-3 (2H)-one 1,1-dioxide (280 mg, 0.92 mmol) in THF (5 mL) was added BH₃-Me₂S (2 M, 2.0 mL, 3.85 mmol) dropwise at 0° C. under nitrogen atmosphere. The solution was heated to reflux and stirred for 3 h. The resulting mixture was cooled to 0° C., quenched with MeOH (1.0 mL), and concentrated under reduced pressure. The residue was purified by silica gel column chromatography, eluting with DCM/MeOH (5:1) to afford the product (131 mg, 49%). LC-MS (ESI): m/z [M−H]—=275.8

### Step 3: cis-3-(1-(tert-butyl)-3-((4-methoxy-1,1-dioxido-2,3-dihydrobenzo[d]isothiazol-5-yl)amino)-1H-pyrazol-5-yl)cyclopentyl isopropylcarbamate

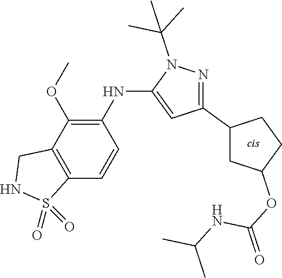 [SMILES: COc1c(Nc2cc(C3CCC(OC(=O)NC(C)C)C3)nn2C(C)(C)C)ccc2c1CNS2(=O)=O]

To a mixture of 5-bromo-4-methoxy-2,3-dihydrobenzo[d]isothiazole 1,1-dioxide (131 mg, 0.47 mmol) and cis-3-(5-amino-1-(tert-butyl)-1H-pyrazol-3-yl)cyclopentyl isopropylcarbamate (146 mg, 0.47 mmol) in andydrous 1,4-dioxane (5 mL) was added Pd₂(dba)₃ (22 mg, 0.02 mmol), XantPhos (27 mg, 0.05 mmol) and K₃PO₄ (300 mg, 1.42 mmol). The reaction mixture was stirred at 90° C. under nitrogen atmosphere for 16 h. The mixture was filtered, and the filtrate was concentrated under reduced pressure. The residue was purified by silica gel column chromatography, eluting with PE/EA (1:1) to afford the product (21 mg, 9%). LC-MS (ESI): m/z [M+H]⁺=506.4.

### Step 4: cis-3-(3-((4-methoxy-1,1-dioxido-2,3-dihydrobenzo[d]isothiazol-5-yl)amino)-1H-pyrazol-5-yl)cyclopentyl isopropylcarbamate

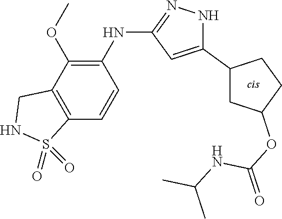 [SMILES: COc1c(Nc2cc(C3CCC(OC(=O)NC(C)C)C3)[nH]n2)ccc2c1CNS2(=O)=O]

To a solution of cis-3-(1-(tert-butyl)-3-((4-methoxy-1,1-dioxido-2,3-dihydrobenzo[d]isothiazol-5-yl)amino)-1H-pyrazol-5-yl)cyclopentyl isopropylcarbamate (21 mg, 0.04 mmol) in DCM (1.5 mL) was added triflic acid (3 drops). The reaction mixture was stirred at room temperature for 2 h. the resulting mixture was neutralized with sat. aq Na₂CO₃ and extracted with DCM (5 mL×3). The combined organic layers were washed with brine, dried over NaSO₄, filtered, and concentrated under reduce pressure. The residue was purified by prep-HPLC (Waters SunFire C18: 19×150 mm, 5 μm, eluting with 30%-50% of acetonitrile (containing 0.1% FA) in water (containing 0.1% FA)) to afford the product (4.0 mg, 21%). 1H NMR (500 MHz, DMSO-d₆) δ=11.92 (s, 1H), 8.19 (s, 2H), 7.53 (t, J=5.1, 1H), 7.37 (d, J=8.5, 1H), 6.94 (d, J=7.3, 1H), 5.88 (s, 1H), 5.00 (m, 1H), 4.40 (d, J=5.1, 2H), 3.80 (s, 3H), 3.62-3.50 (m, 1H), 3.13-2.99 (m, 1H), 2.46 (m, 1H), 2.02 (m, 1H), 1.96-1.85 (m, 1H), 1.71 (m, 2H), 1.60 (m, 1H), 1.09-0.93 (m, 6H). LC-MS (ESI): m/z [M+H]⁺=450.4.

### Example 11: cis-3-(3-((2-methyl-1,1-dioxido-2,3-dihydrobenzo[d]isothiazol-5-yl)amino)-1H-pyrazol-5-yl)cyclopentyl isopropylcarbamate

### Step 1:5-bromo-2-methyl-2,3-dihydrobenzo[d]isothiazole 1,1-dioxide

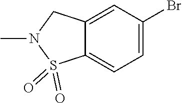

To a mixture of 5-bromo-2,3-dihydrobenzo[d]isothiazole 1,1-dioxide (200 mg, 0.81 mmol) and K₂CO₃ (223.6 mg, 1.62 mmol) in ethanol (15 mL) was added iodomethane (172.5 mg, 1.22 mmol). The reaction was stirred in a round bottom flask at 50° C. for overnight. The mixture was evaporated in vacuum, and the residue was purified with silica gel column chromatography (PE:EA=3:1 to 1:2 gradient elution) to give the product (0.18 g, 64%). LC-MS (ESI): m/z [M+H]⁺=262.1.

### Step 2: cis-3-(1-(tert-butyl)-5-((2-methyl-1,1-dioxido-2,3-dihydrobenzo[d]isothiazol-5-yl)amino)-1H-pyrazol-3-yl)cyclopentyl isopropylcarbamate

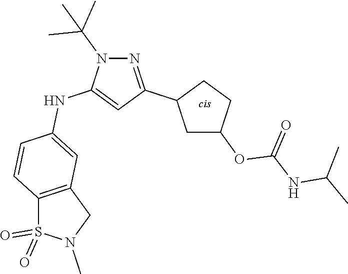 [SMILES: CC(C)NC(=O)OC1CCC(c2cc(Nc3ccc4c(c3)CN(C)S4(=O)=O)n(C(C)(C)C)n2)C1]

To a mixture of cis-3-(5-amino-1-(tert-butyl)-1H-pyrazol-3-yl)cyclopentyl isopropylcarbamate (177 mg, 0.575 mmol) and 5-bromo-2-methyl-2,3-dihydrobenzo[d]isothiazole 1,1-dioxide (180 mg, 0.69 mmol) in andydrous 1,4-dioxane (35 mL) was added Pa₂dba₃ (52.7 mg, 0.0575 mmol), Xantphos (66.5 mg, 0.115 mmol) and K₃PO₄ (365.7 mg, 1.725 mmol). The reaction mixture was stirred at 90° C. under nitrogen atmosphere for 16 h. The mixture was filtered, and the filtrate was concentrated under reduced pressure. The residue was purified by silica gel column chromatography (PE:EA=3:1~ 1:2 gradient elution) to give the product (0.18 g, 64%). LC-MS (ESI): m/z [M+H]⁺=490.5.

### Step 3: cis-3-(3-((2-methyl-1,1-dioxido-2,3-dihydrobenzo[d]isothiazol-5-yl)amino)-1H-pyrazol-5-yl)cyclopentyl isopropylcarbamate

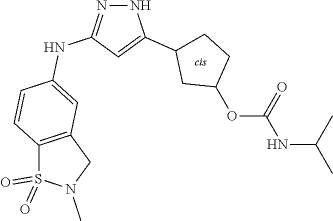 [SMILES: CC(C)NC(=O)OC1CCC(c2cc(Nc3ccc4c(c3)CN(C)S4(=O)=O)n[nH]2)C1]

To a solution of cis-3-(1-(tert-butyl)-5-((2-methyl-1,1-dioxido-2,3-dihydrobenzo[d]isothiazol-5-yl)amino)-1H-pyrazol-3-yl)cyclopentyl isopropylcarbamate (170 mg, 0.348 mmol) in DCM (8 mL) was added triflic acid (1.0 mL) at 25° C. The mixture was stirred at 25° C. for 2 h. The mixture was quenched with NH₃ in MeOH (7 N, 4.0 mL) and evaporated under reduced pressure. The residue was purified by Prep-HPLC (Sunfire C18 column, eluting with 34%-44% a gradient of acetonitrile/water containing 0.1% FA, at flow rate of 17 mL/min) to afford the product (109 mg, 72%). ¹H NMR (500 MHz, DMSO-d₆) δ 11.93 (s, 1H), 9.05 (s, 1H), 7.58 (d, J=8.6 Hz, 1H), 7.48 (d, J=9.8 Hz, 1H), 7.33 (d, J=8.5 Hz, 1H), 6.95 (d, J=7.4 Hz, 1H), 5.71 (s, 1H), 5.04-4.95 (m, 1H), 4.28 (s, 2H), 3.58 (td, J=12.8, 6.2 Hz, 1H), 3.12-3.00 (m, 1H), 2.74 (s, 3H), 2.46 (dd, J=13.8, 7.1 Hz, 1H), 2.06-1.98 (m, 1H), 1.96-1.86 (m, 1H), 1.78-1.66 (m, 2H), 1.65-1.55 (m, 1H), 1.03 (d, J=6.3 Hz, 6H). LC-MS (ESI): m/z [M+H]⁺=434.3.

### Example 12: cis-3-(3-((6-fluoro-1,1-dioxido-2,3-dihydrobenzo[d]isothiazol-5-yl)amino)-1H-pyrazol-5-yl)cyclopentyl isopropylcarbamate

### Step 1: methyl 5-bromo-2-(chlorosulfonyl)-4-fluorobenzoate

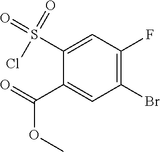 [SMILES: COC(=O)c1cc(Br)c(F)cc1S(=O)(=O)Cl]

To a suspension of methyl 2-amino-5-bromo-4-fluorobenzoate (3.0 g, 12.1 mmol) in acetic acid (20 mL) and hydrochloric acid (12 M, 54 mL), a solution of sodium nitrite (918.4 mg, 13.31 mmol) in 8 mL water was added dropwise at 0° C. The resulting mixture was kept below 5° C. and stirred for 30 mins after addition. To the reaction mixture CuCl₂ (813 mg, 6.05 mmol), sodium bisulfite (18.9 g, 181.5 mmol) and hydrochloric acid solution (6 M, 88 mL) were added sequentially. The reaction mixture was warmed to room temperature and stirred for 3 h. The resulting mixture was extracted with EA (50 mL×3). The combined organic layers were washed with brine, dried over Na₂SO₄, and filtered. The filtrated was concentrated and dried over in vacuum to afford the crude product (3.0 g), which was used in the next step without further purification.

### Step 2:5-bromo-6-fluorobenzo[d]isothiazol-3 (2H)-one 1,1-dioxide

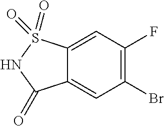 [SMILES: O=C1NS(=O)(=O)c2cc(F)c(Br)cc21]

To a mixture of methyl 5-bromo-2-(chlorosulfonyl)-4-fluorobenzoate (3 g crude) was dissolved in THF (10 mL) was added, aqueous ammonium (25%, 20 mL). The reaction mixture was stirred at room temperature for 16 h. The resulting mixture was concentrated under vacuum, and the residue was triturated with EA and filtered to afford the crude product (2.0 g). LC-MS (ESI): m/z [M−H]⁻=277.9.

### Step 3:5-bromo-6-fluoro-2,3-dihydrobenzo[d]isothiazole 1,1-dioxide

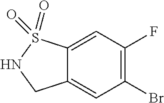 [SMILES: O=S1(=O)NCc2cc(Br)c(F)cc21]

To a stirring solution of 5-bromo-6-fluoro-2,3-dihydrobenzo[d]isothiazole 1,1-dioxide (300 mg, 1.08 mmol) in THF (10 mL) was added BH₃-Me₂S (2 M, 2.7 mL, 5.4 mmol) dropwise at 0° C. under nitrogen atmosphere. The solution was heated to reflux and stirred for 16 h. The resulting mixture was cooled to 0° C., quenched with MeOH (10 mL), and concentrated under reduced pressure. The residue was purified by silica gel column chromatography, eluting with PE/EA (1:1) to afford the product (260 mg, 91%). LC-MS (ESI): m/z [M+H]⁺=266/268.

### Step 4: cis-3-(1-(tert-butyl)-5-((6-fluoro-1,1-dioxido-2,3-dihydrobenzo[d]isothiazol-5-yl)amino)-1H-pyrazol-3-yl)cyclopentyl isopropylcarbamate

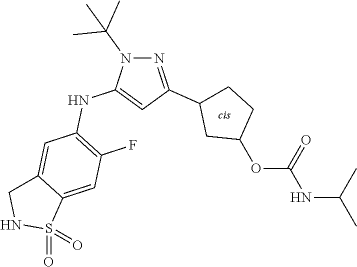 [SMILES: CC(C)NC(=O)OC1CCC(c2cc(Nc3cc4c(cc3F)S(=O)(=O)NC4)n(C(C)(C)C)n2)C1]

To A mixture of cis-3-(5-amino-1-(tert-butyl)-1H-pyrazol-3-yl)cyclopentyl isopropylcarbamate (100 mg, 0.324 mmol) and 5-bromo-6-fluoro-2,3-dihydrobenzo[d]isothiazole 1,1-dioxide (86 mg, 0.324 mmol) in andydrous 1,4-dioxane (25 mL) was added Pa₂dba₃ (30 mg, 0.0324 mmol), Xantphos (37.5 mg, 0.0648 mmol) and K₃PO₄ (206 mg, 0.972 mmol), he reaction mixture was stirred at 90° C. under nitrogen atmosphere for 16 h. The mixture was filtered, and the filtrate was concentrated under reduced pressure. The residue was purified by silica gel column chromatography (PE:EA=3:1~1:2 gradient elution) to give the product (0.1 g, 62.5%). LC-MS (ESI): m/z [M+H]⁺=494.3.

### Step 5: cis-3-(3-((6-fluoro-1,1-dioxido-2,3-dihydrobenzo[d]isothiazol-5-yl)amino)-1H-pyrazol-5-yl)cyclopentyl isopropylcarbamate

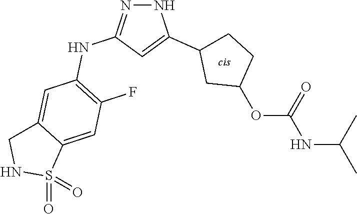 [SMILES: CC(C)NC(=O)OC1CCC(c2cc(Nc3cc4c(cc3F)S(=O)(=O)NC4)n[nH]2)C1]

To a solution of cis-3-(1-(tert-butyl)-5-((6-fluoro-1,1-dioxido-2,3-dihydrobenzo[d]isothiazol-5-yl)amino)-1H-pyrazol-3-yl)cyclopentyl isopropylcarbamate (90 mg, 0.18 mmol) in DCM (8 mL) was added triflic acid (0.5 mL) at 25° C. The mixture was stirred at 25° C. for 1 h. Reaction was monitored by LCMS. The mixture was quenched with NH₃ in MeOH (2.0 mL, 7N) and evaporated under reduced pressure. The residue was purified by Prep-HPLC (Sunfire C18 column, eluting with eluting with 31%-50% a gradient of acetonitrile/water containing 0.1% FA at flow rate of 17 mL/min) to afford the product in racemic form, which was further purified with chiral Prep-HPLC to give:

Enantiomer 1 (Example 12a, 100% ee); Retention time: 6.78 min. ¹H NMR (500 MHz, DMSO-d₆) δ 11.99 (s, 1H), 8.74 (s, 1H), 8.13 (d, J=4.7 Hz, 1H), 7.64 (d, J=10.0 Hz, 2H), 6.94 (d, J=7.3 Hz, 1H), 5.85 (s, 1H), 5.00 (s, 1H), 4.30 (s, 2H), 3.58 (dd, J=13.4, 6.7 Hz, 1H), 3.12-3.02 (m, 1H), 2.46 (dd, J=14.0, 7.2 Hz, 1H), 2.02 (dd, J=16.0, 7.6 Hz, 1H), 1.96-1.86 (m, 1H), 1.78-1.65 (m, 2H), 1.60 (d, J=13.6 Hz, 1H), 1.03 (d, J=6.3 Hz, 6H). LC-MS (ESI): m/z [M+H]⁺=438.3.

Enantiomer 2 (Example 12b, 100% ee); Retention time: 8.10 min. ¹H NMR (500 MHz, DMSO-d₆) δ 11.99 (s, 1H), 8.74 (s, 1H), 8.13 (d, J=4.7 Hz, 1H), 7.64 (d, J=10.0 Hz, 2H), 6.94 (d, J=7.3 Hz, 1H), 5.85 (s, 1H), 5.00 (s, 1H), 4.30 (s, 2H), 3.58 (dd, J=13.4, 6.7 Hz, 1H), 3.12-3.02 (m, 1H), 2.46 (dd, J=14.0, 7.2 Hz, 1H), 2.02 (dd, J=16.0, 7.6 Hz, 1H), 1.96-1.86 (m, 1H), 1.78-1.65 (m, 2H), 1.60 (d, J=13.6 Hz, 1H), 1.03 (d, J=6.3 Hz, 6H). LC-MS (ESI): m/z [M+H]⁺=438.3.

Chiral analytical method: Column: CHIRALPAK IF 4.6 mm×150 mm, 5 μm; Mobile phase: A for Hexane and B for EtOH (0.1% 2M NH₃ MeOH); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IF 20 mm×250 mm, 5 μm; Mobile phase: A for Hexane and B for EtOH (0.2% 2M NH₃ MeOH); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 13: cis-3-(3-((7-fluoro-3,3-dimethyl-1,1-dioxido-2,3-dihydrobenzo[d]isothiazol-6-yl)amino)-1H-pyrazol-5-yl)cyclopentyl isopropylcarbamate

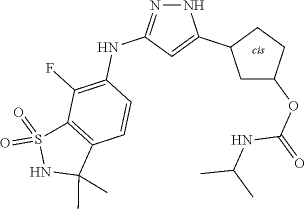 [SMILES: CC(C)NC(=O)OC1CCC(c2cc(Nc3ccc4c(c3F)S(=O)(=O)NC4(C)C)n[nH]2)C1]

The titled compound was synthesized in the procedures similar to Example 8. ¹H NMR (500 MHz, DMSO-d₆) δ=11.92 (s, 1H), 8.48 (s, 1H), 8.30 (t, J=7.5, 1H), 8.02 (s, 1H), 7.29 (d, J=8.5, 1H), 6.94 (d, J=7.5, 1H), 5.77 (s, 1H), 5.00 (m, 1H), 3.58 (m, 1H), 3.12-2.97 (m, 1H), 2.46 (m, 1H), 2.02 (m, 1H), 1.96-1.85 (m, 1H), 1.70 (m, 2H), 1.59 (m, 1H), 1.50 (s, 6H), 1.09-0.95 (m, 6H). LC-MS (ESI): m/z [M+H]⁺=466.3.

### Example 14: cis-3-(3-((6-fluoro-3,3-dimethyl-1,1-dioxido-2,3-dihydrobenzo[d]isothiazol-5-yl)amino)-1H-pyrazol-5-yl)cyclopentyl isopropylcarbamate

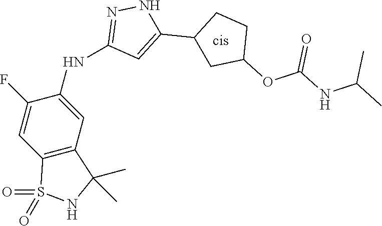 [SMILES: CC(C)NC(=O)OC1CCC(c2cc(Nc3cc4c(cc3F)S(=O)(=O)NC4(C)C)n[nH]2)C1]

The titled compound was synthesized in the procedures similar to Example 8. ¹H NMR (500 MHz, DMSO-d₆) δ 12.06 (s, 1H), 8.81 (d, J=2.2 Hz, 1H), 8.28 (d, J=5.2 Hz, 1H), 7.73 (s, 1H), 7.58 (d, J=10.1 Hz, 1H), 6.93 (d, J=7.3 Hz, 1H), 5.84 (s, 1H), 4.99 (d, J=2.9 Hz, 1H), 3.58 (qd, J=12.2, 5.3 Hz, 1H), 3.11-2.99 (m, 1H), 2.49-2.40 (m, 1H), 2.06-1.97 (m, 1H), 1.96-1.85 (m, 1H), 1.78-1.63 (m, 2H), 1.63-1.54 (m, 1H), 1.47 (s, 6H), 1.03 (d, J=6.1 Hz, 6H). LC-MS (ESI): m/z [M+H]⁺=466.3.

### Example 15: cis-3-(3-((6-methoxy-1,1-dioxido-2,3-dihydrobenzo[d]isothiazol-5-yl)amino)-1H-pyrazol-5-yl)cyclopentyl isopropylcarbamate

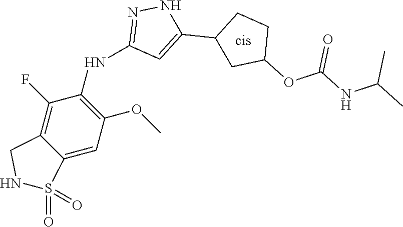 [SMILES: COc1cc2c(c(F)c1Nc1cc(C3CCC(OC(=O)NC(C)C)C3)[nH]n1)CNS2(=O)=O]

The titled compound was synthesized in the procedures similar to Example 9. ¹H NMR (500 MHz, DMSO-d₆) δ 11.93 (s, 1H), 8.02 (d, J=19.2 Hz, 2H), 7.48 (s, 1H), 7.22 (s, 1H), 6.95 (d, J=7.3 Hz, 1H), 5.90 (s, 1H), 4.99 (s, 1H), 4.26 (s, 2H), 3.94 (s, 3H), 3.58 (dd, J=13.1, 6.5 Hz, 1H), 3.06 (dd, J=16.9, 8.4 Hz, 1H), 2.49-2.40 (m, 1H), 2.06-1.97 (m, 1H), 1.95-1.85 (m, 1H), 1.79-1.64 (m, 2H), 1.64-1.53 (m, 1H), 1.03 (d, J=6.2 Hz, 6H). LC-MS (ESI): m/z [M+H]⁺=450.4.

### Example 16: cis-3-(3-((1,1-dioxido-3-oxo-2,3-dihydrobenzo[d]isothiazol-6-yl)amino)-1H-pyrazol-5-yl)cyclopentyl isopropylcarbamate

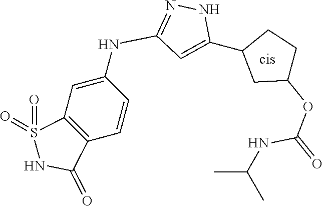 [SMILES: CC(C)NC(=O)OC1CCC(c2cc(Nc3ccc4c(c3)S(=O)(=O)NC4=O)n[nH]2)C1]

The titled compound was synthesized in the procedures similar to Example 3. ¹H NMR (500 MHz, DMSO-d₆) δ 12.11 (s, 1H), 9.72 (s, 1H), 8.12 (s, 1H), 7.77 (d, J=8.6 Hz, 1H), 7.52 (d, J=8.3 Hz, 1H), 6.95 (d, J=7.5 Hz, 1H), 5.75 (s, 1H), 5.10-4.93 (m, 1H), 3.70-3.40 (m, 1H), 3.19-2.95 (m, 1H), 2.39-2.21 (m, 2H), 2.11-1.98 (m, 1H), 1.92 (d, J=7.2 Hz, 1H), 1.72 (s, 2H), 1.61 (s, 1H), 1.03 (d, J=5.4 Hz, 6H). LC-MS (ESI): m/z [M+H]⁺=434.4.

### Example 17: cis-3-(3-((1,1-dioxido-3-oxo-2,3-dihydrobenzo[d]isothiazol-5-yl)amino)-1H-pyrazol-5-yl)cyclopentyl isopropylcarbamate

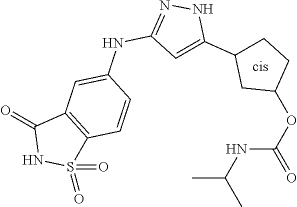 [SMILES: CC(C)NC(=O)OC1CCC(c2cc(Nc3ccc4c(c3)C(=O)NS4(=O)=O)n[nH]2)C1]

The titled compound was synthesized in the procedures similar to Example 3. ¹H NMR (500 MHz, DMSO-d₆) δ 12.05 (s, 1H), 9.45 (s, 1H), 8.02 (s, 1H), 7.86 (d, J=8.6 Hz, 1H), 7.65 (d, J=8.5 Hz, 1H), 6.95 (d, J=6.2 Hz, 1H), 5.72 (s, 1H), 5.10-4.90 (m, 1H), 3.61-3.45 (m, 3H), 3.15-3.01 (m, 1H), 2.08-1.98 (m, 1H), 1.95-1.85 (m, 1H), 1.79-1.66 (m, 2H), 1.65-1.55 (m, 1H), 1.03 (d, J=4.4 Hz, 6H).

LC-MS (ESI): m/z [M+H]⁺=434.3.

### Example 18: cis-3-(3-((1,1,1′,1′-tetraoxido-2′,3′-dihydro-3H-[2,5′-bibenzo[d]isothiazol]-5-yl)amino)-1H-pyrazol-5-yl)cyclopentyl isopropylcarbamate

### Step 1: cis-3-(1-(tert-butyl)-5-((1,1,1′,1′-tetraoxido-2′,3′-dihydro-3H-[2,5′-bibenzo[d]isothiazol]-5-yl)amino)-1H-pyrazol-3-yl)cyclopentyl isopropylcarbamate

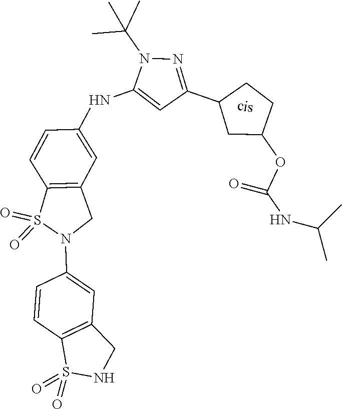 [SMILES: CC(C)NC(=O)OC1CCC(c2cc(Nc3ccc4c(c3)CN(c3ccc5c(c3)CNS5(=O)=O)S4(=O)=O)n(C(C)(C)C)n2)C1]

To a mixture of 5-bromo-2,3-dihydrobenzo[d]isothiazole 1,1-dioxide (100 mg, 0.40 mmol) and cis-3-(5-amino-1-(tert-butyl)-1H-pyrazol-3-yl)cyclopentyl isopropylcarbamate (123.2 mg, 0.40 mmol) in andydrous 1,4-dioxane (10 mL) was added Pd₂(dba)₃ (36.7 mg, 0.04 mmol), XantPhos (46.2 mg, 0.08 mmol) and K₃PO₄ (254.4 mg, 1.2 mmol). The reaction mixture was stirred at 90° C. under nitrogen atmosphere for 16 h. The mixture was filtered, and the filtrate was concentrated under reduced pressure. The residue was purified by silica gel column chromatography, eluting with DCM/MeOH (20:1) to afford the product (30 mg, 10%). LC-MS (ESI): m/z [M+H]⁺=643.2.

### Step 2: cis-3-(3-((1,1,1′,1′-tetraoxido-2′,3′-dihydro-3H-[2,5′-bibenzo[d]isothiazol]-5-yl)amino)-1H-pyrazol-5-yl)cyclopentyl isopropylcarbamate

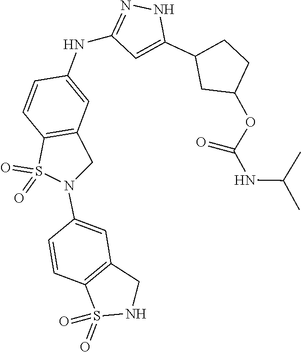 [SMILES: CC(C)NC(=O)OC1CCC(c2cc(Nc3ccc4c(c3)CN(c3ccc5c(c3)CNS5(=O)=O)S4(=O)=O)n[nH]2)C1]

A solution of cis-3-(1-(tert-butyl)-5-((1,1,1′,1′-tetraoxido-2′,3′-dihydro-3H-[2,5′-bibenzo[d]isothiazol]-5-yl)amino)-1H-pyrazol-3-yl)cyclopentyl isopropylcarbamate (30 mg, 0.05 mmol) in formic acid (5 mL) was stirred at 75° C. for 16 h. The solution was concentrated under reduced pressure. The residue was purified by prep HPLC (Waters SunFire C18: RD-CO-095 column, eluting with 25%-55% of acetonitrile (containing 0.1% FA) in water (containing 0.1% FA) to afford the product (5 mg, 19%). ¹H NMR (500 MHz, DMSO-d₆) δ 12.00 (s, 1H), 9.25 (s, 1H), 7.90 (d, J=8.6 Hz, 1H), 7.76 (d, J=8.8 Hz, 2H), 7.65 (s, 1H), 7.56 (d, J=8.6 Hz, 1H), 7.49 (s, 1H), 7.43 (d, J=8.1 Hz, 1H), 6.95 (d, J=7.2 Hz, 1H), 5.74 (s, 1H), 5.06 (s, 2H), 5.04-4.96 (m, 1H), 4.43 (s, 2H), 3.65-3.53 (m, 1H), 3.12-3.05 (m, 1H), 2.47-2.44 (m, 1H), 2.08-2.01 (m, 1H), 1.96-1.85 (m, 1H), 1.79-1.67 (m, 2H), 1.65-1.58 (m, 1H), 1.03 (d, J=5.8 Hz, 6H). LC-MS (ESI): m/z [M+H]⁺=587.4.

### Example 19: cis-1-(3-(3-((1,1-dioxido-2,3-dihydrobenzo[d]isothiazol-5-yl)amino)-1H-pyrazol-5-yl)cyclopentyl)-3-isopropylurea

### Step 1: benzyl(3-(3-(benzylamino)cyclopentyl)-1-(tert-butyl)-1H-pyrazol-5-yl)carbamate

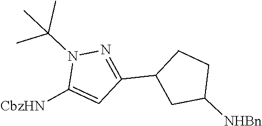

To a solution of benzyl(1-(tert-butyl)-3-(3-oxocyclopentyl)-1H-pyrazol-5-yl)carbamate (1.0 g, 2.8 mmol) in 1, 2-dichloroethane (100 mL) was added benzylamine (360 mg, 3.36 mmol), potassium acetate (1.372 g, 14 mmol) and sodium triacetoxyborohydride (1.19 g, 5.6 mmol). The resulting mixture was stirred at 50° C. for 2 h. The mixture was added water (100 mL) and the layer separated. The aqueous layer was extracted with DCM (50 mL×3). The combined organic layers were dried over Na₂SO₄, filtered and concentrated. The residue was purified with silica gel column chromatography (PE:EA=10:1~4:1 gradient elution) to give the product (1.12 g, 90%). LC-MS (ESI): m/z [M+H]⁺=447.4.

### Step 2: benzyl(3-(3-(1-benzyl-3-isopropylureido) cyclopentyl)-1-(tert-butyl)-1H-pyrazol-5-yl)carbamate

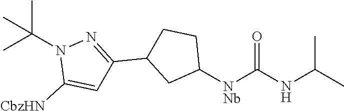

To a mixture of benzyl(3-(3-(benzylamino)cyclopentyl)-1-(tert-butyl)-1H-pyrazol-5-yl)carbamate (500 mg, 1.12 mmol) and DIPEA (434 mg, 3.36 mmol) in DCM (20 mL) was added 2-isocyanatopropane (286 mg, 3.36 mmol). The mixture was stirred at 25° C. for 16 h. The mixture was concentrated in vacuum to afford the crude product, which was further purified with silica gel column chromatography (PE:EA=2:1~1:3 gradient elution) to give the product (550 mg, 92%). LC-MS (ESI): m/z [M+H]⁺=532.5.

### Step 3:1-(3-(5-amino-1-(tert-butyl)-1H-pyrazol-3-yl)cyclopentyl)-3-isopropylurea

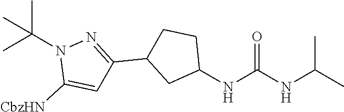 [SMILES: CC(C)NC(=O)NC1CCC(c2cc(NC(=O)OCc3ccccc3)n(C(C)(C)C)n2)C1]

To a suspension of benzyl(3-(3-(benzylamino)cyclopentyl)-1-(tert-butyl)-1H-pyrazol-5-yl)carbamate (550 mg, 1.036 mmol) and Pd/C (100 mg, 10%, wet) in THF/H₂O (20 mL/2 mL) was degassed and purged with hydrogen 3 times. The resulting mixture was stirred at room temperature under a hydrogen balloon for 15 h. The mixture was filtered through a pad of Celite and the filter cake was washed with MeOH (20 mL). The filtrate was concentrated under vacuum to obtain the product (310.0 mg, 97%). LC-MS (ESI): m/z [M+H]⁺=308.3.

### Step 4: cis-1-(3-(1-(tert-butyl)-5-((1,1-dioxido-2,3-dihydrobenzo[d]isothiazol-5-yl)amino)-1H-pyrazol-3-yl)cyclopentyl)-3-isopropylurea

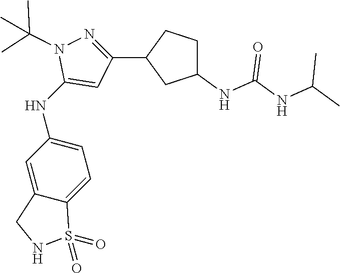 [SMILES: CC(C)NC(=O)NC1CCC(c2cc(Nc3ccc4c(c3)CNS4(=O)=O)n(C(C)(C)C)n2)C1]

To a mixture of cis-1-(3-(5-amino-1-(tert-butyl)-1H-pyrazol-3-yl)cyclopentyl)-3-isopropylurea (100 mg, 0.326 mmol), 5-bromo-2,3-dihydrobenzo[d]isothiazole 1,1-dioxide (80 mg, 0.326 mmol) in andydrous 1,4-dioxane (20 mL) was added Pa₂dba₃ (30 mg, 0.033 mmol), Xantphos (38 mg, 0.063 mmol) and K₃PO₄ (207.3 mg, 0.98 mmol). The reaction mixtrue was stirred in a round bottom flask at 90° C. under nitrogen for 16 h. The mixture was concentrated in vacuum and the residue was further purified with silica gel column chromatography (PE:EA=3:1~1:2 gradient elution) to give the product (0.1 g, 65%). LC-MS (ESI): m/z [M+H]⁺=475.3.

### Step 5: cis-1-(3-(3-((1,1-dioxido-2,3-dihydrobenzo[d]isothiazol-5-yl)amino)-1H-pyrazol-5-yl)cyclopentyl)-3-isopropylurea

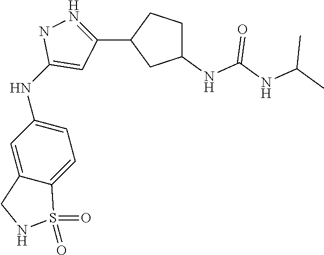

To a solution of cis-1-(3-(1-(tert-butyl)-3-((1,1-dioxido-2,3-dihydrobenzo[d]isothiazol-5-yl)amino)-1H-pyrazol-5-yl)cyclopentyl)-3-isopropylurea (100 mg, 0.21 mmol) in DCM (8 mL) was added trifluoromethanesulfonic acid (1.0 mL). The mixture was stirred at 25° C. for 30 min. The mixture was quenched with NH₃ in MeOH (7 N, 2.0 mL) and concentrated under reduced pressure. The residue was purified with Prep-HPLC (Sunfire C18 column, eluting with 34%-44% a gradient of acetonitrile/water containing 0.1% FA, at flow rate of 17 mL/min) to afford the product (20 mg, 23%). 1H NMR (500 MHz, DMSO-d₆) δ 11.90 (s, 1H), 9.00 (s, 1H), 7.53 (d, J=8.6 Hz, 1H), 7.46 (s, 2H), 7.30 (d, J=8.1 Hz, 1H), 5.80 (dd, J=23.0, 7.2 Hz, 1H), 5.69 (d, J=3.6 Hz, 1H), 5.51 (dd, J=23.0, 7.6 Hz, 1H), 4.29 (s, 2H), 4.08-3.91 (m, 1H), 3.71-3.58 (m, 1H), 3.21-2.97 (m, 1H), 2.38-2.27 (m, 1H), 2.10-1.75 (m, 2H), 1.73-1.54 (m, 1H), 1.50-1.31 (m, 2H), 1.01 (dd, J=6.5, 1.8 Hz, 6H). LC-MS (ESI): m/z [M+H]⁺=419.4.

### Example 20: cis-3-(3-((1,1-dioxido-6-(trifluoromethyl)-2,3-dihydrobenzo[d]isothiazol-5-yl)amino)-1H-pyrazol-5-yl)cyclopentyl isopropylcarbamate

### Step 1: methyl 2-amino-5-bromo-4-(trifluoromethyl)benzoate

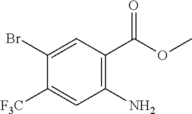 [SMILES: COC(=O)c1cc(Br)c(C(F)(F)F)cc1N]

To a solution of methyl 2-amino-5-bromo-4-(trifluoromethyl)benzoate (6 g, 27.4 mmol) in DMF (100 mL) was added NBS (4.8 g, 27.4 mmol). The resulting solution was stirred at room temperature for 16 h. The solution was poured into 200 mL water and extracted with EA (100 mL×3). The combined EA layers were dried and concentrated under vacuum. The residue was purified with silica gel column chromatography, eluting with PE/EtOAc (10: 1~2:1) to afford the product (5.1 g, 63%). LC-MS (ESI): m/z [M+H]⁺=298.1

### Step 2:5-bromo-6-(trifluoromethyl)benzo[d]isothiazol-3 (2H)-one 1,1-dioxide

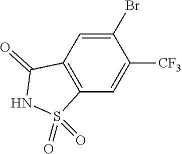 [SMILES: O=C1NS(=O)(=O)c2cc(C(F)(F)F)c(Br)cc21]

The titled compound was synthesized in the procedures similar to Example 1, step 10 to step 11.

LC-MS (ESI): m/z [M−H]⁻=327.9.

### Step 3:5-bromo-6-(trifluoromethyl)-2,3-dihydrobenzo[d]isothiazole 1,1-dioxide

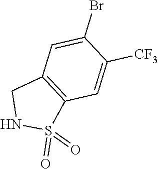 [SMILES: O=S1(=O)NCc2cc(Br)c(C(F)(F)F)cc21]

The titled compound was synthesized in the procedures similar to Example 1 step 12. LC-MS (ESI): m/z [M+H]⁺=316.3.

### Step 4: cis-3-(3-((1,1-dioxido-6-(trifluoromethyl)-2,3-dihydrobenzo[d]isothiazol-5-yl)amino)-1H-pyrazol-5-yl)cyclopentyl isopropylcarbamate

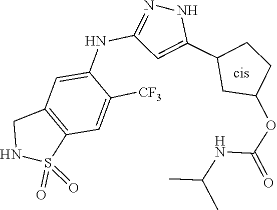 [SMILES: CC(C)NC(=O)OC1CCC(c2cc(Nc3cc4c(cc3C(F)(F)F)S(=O)(=O)NC4)n[nH]2)C1]

The titled compound was synthesized in the procedures similar to Example 1, step 13 to step 14. ¹H NMR (500 MHz, DMSO-d₆) δ 12.26 (s, 1H), 7.93 (s, 2H), 7.81-7.68 (m, 2H), 6.93 (d, J=5.0 Hz, 1H), 6.00 (s, 1H), 5.05-4.96 (m, 1H), 4.34 (d, J=5.0 Hz, 2H), 3.63-3.51 (m, 1H), 3.14-3.04 (m, 1H), 2.47-2.43 (m, 1H), 2.09-1.99 (m, 1H), 1.96-1.85 (m, 1H), 1.78-1.68 (m, 2H), 1.65-1.57 (m, 1H), 1.07-0.96 (m, 6H).

LC-MS (ESI): m/z [M+H]⁺=488.2.

### Example 21: cis-3-(3-((3,3-dimethyl-1,1-dioxido-6-(trifluoromethyl)-2,3-dihydrobenzo[d]isothiazol-5-yl)amino)-1H-pyrazol-5-yl)cyclopentyl isopropylcarbamate

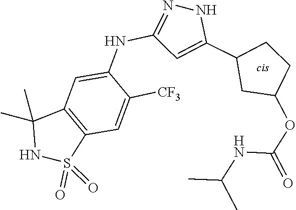 [SMILES: CC(C)NC(=O)OC1CCC(c2cc(Nc3cc4c(cc3C(F)(F)F)S(=O)(=O)NC4(C)C)n[nH]2)C1]

The titled compound was synthesized in the procedures similar to Example 8. ¹H NMR (500 MHz, DMSO-d₆) δ 12.26 (s, 1H), 8.06 (s, 1H), 8.00 (s, 1H), 7.88 (d, J=10.0 Hz, 2H), 6.93 (d, J=5.0 Hz, 1H), 6.01 (s, 1H), 5.02-4.95 (m, 1H), 3.61-3.53 (m, 1H), 3.12-3.03 (m, 1H), 2.46-2.41 (m, 1H), 2.08-1.99 (m, 1H), 1.95-1.86 (m, 1H), 1.77-1.66 (m, 2H), 1.65-1.58 (m, 1H), 1.46 (m, 6H), 1.06-0.95 (m, 6H).

LC-MS (ESI): m/z [M+H]⁺=516.2.

### Example 22: cis-3-(3-((1,1-dioxido-4-(trifluoromethyl)-2,3-dihydrobenzo[d]isothiazol-5-yl)amino)-1H-pyrazol-5-yl)cyclopentyl isopropylcarbamate

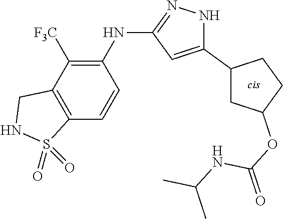 [SMILES: CC(C)NC(=O)OC1CCC(c2cc(Nc3ccc4c(c3C(F)(F)F)CNS4(=O)=O)n[nH]2)C1]

The titled compound was synthesized in the procedures similar to Example 1. ¹H NMR (500 MHz, DMSO-d₆) δ 12.35 (s, 1H), 7.95 (s, 1H), 7.84-7.78 (m, 1H), 7.76-7.65 (m, 2H), 6.64 (d, J=10 Hz, 1H), 5.98 (s, 1H), 5.00 (d, J=5.0 Hz, 1H), 4.46 (s, 1H), 3.55-3.51 (m, 1H), 3.14-3.05 (m, 1H), 2.48-2.43 (m, 1H), 2.09-1.99 (m, 1H), 1.86-1.86 (m, 1H), 1.79-1.68 (m, 2H), 1.66-1.58 (m, 1H), 1.07-0.97 (m, 6H).

LC-MS (ESI): m/z [M+H]⁺=488.2.

### Example 23: cis-3-(3-((6-methoxy-3,3-dimethyl-1,1-dioxido-2,3-dihydrobenzo[d]isothiazol-5-yl)amino)-1H-pyrazol-5-yl)cyclopentyl isopropylcarbamate

### Step 1:5-bromo-6-methoxybenzo[d]isothiazol-3 (2H)-one 1,1-dioxide

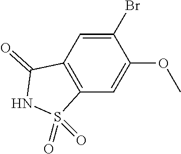

The titled compound was synthesized in the procedures similar to Example 1, step 10 to step 11.

LC-MS (ESI): m/z [M−H]⁻=290.0.

### Step 2: cis-3-(3-((6-methoxy-3,3-dimethyl-1,1-dioxido-2,3-dihydrobenzo[d]isothiazol-5-yl)amino)-1H-pyrazol-5-yl)cyclopentyl isopropylcarbamate

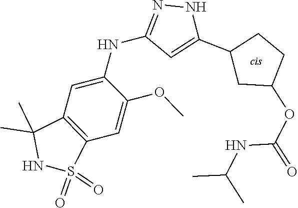 [SMILES: COc1cc2c(cc1Nc1cc(C3CCC(OC(=O)NC(C)C)C3)[nH]n1)C(C)(C)NS2(=O)=O]

The titled compound was synthesized in the procedures similar to Example 6. ¹H NMR (500 MHz, DMSO-d₆) δ 11.98 (s, 1H), 8.10 (s, 1H), 7.55 (s, 1H), 7.15 (s, 1H), 6.94 (d, J=7.4 Hz, 1H), 5.90 (s, 1H), 4.99 (s, 1H), 3.69-3.34 (m, 5H), 3.10-2.98 (m, 1H), 2.45-2.37 (m, 1H), 2.07-1.97 (m, 1H), 1.97-1.86 (m, 1H), 1.79-1.66 (m, 2H), 1.63-1.53 (m, 1H), 1.47 (s, 6H), 1.03 (d, J=5.8 Hz, 6H). LC-MS (ESI): m/z [M+H]⁺=478.2.

### Example 24: cis-3-(3-((4-methoxy-3,3-dimethyl-1,1-dioxido-2,3-dihydrobenzo[d]isothiazol-5-yl)amino)-1H-pyrazol-5-yl)cyclopentyl isopropylcarbamate

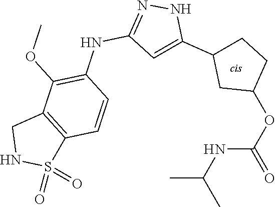 [SMILES: COc1c(Nc2cc(C3CCC(OC(=O)NC(C)C)C3)[nH]n2)ccc2c1CNS2(=O)=O]

The titled compound was synthesized in the procedures similar to Example 6. ¹H NMR (500 MHz, DMSO-d₆) δ=11.96 (s, 1H), 8.15 (s, 1H), 8.06 (s, 1H), 7.62 (s, 1H), 7.32 (d, J=8.6, 1H), 6.94 (d, J=7.6, 1H), 5.87 (s, 1H), 4.99 (s, 1H), 3.78 (s, 3H), 3.60 (m, 1H), 3.11-3.00 (m, 1H), 2.47 (m, 1H), 2.03 (m, 1H), 1.97-1.85 (m, 1H), 1.72 (m, 2H), 1.60 (m, 7H), 1.07-0.93 (m, 6H). LC-MS (ESI): m/z [M+H]⁺=478.2.

### Example 25: cis-3-(3-((6-methyl-1,1-dioxido-2,3-dihydrobenzo[d]isothiazol-5-yl)amino)-1H-pyrazol-5-yl)cyclopentyl isopropylcarbamate

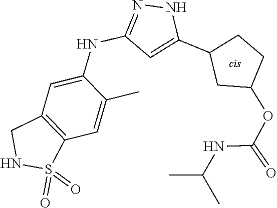 [SMILES: Cc1cc2c(cc1Nc1cc(C3CCC(OC(=O)NC(C)C)C3)[nH]n1)CNS2(=O)=O]

The titled compound was synthesized in the procedures similar to Example 1. ¹H NMR (500 MHz, DMSO-d₆) δ 11.96 (s, 1H), 7.82 (s, 1H), 7.74 (s, 1H), 7.46-7.40 (m, 2H), 6.94 (d, J=7.3 Hz, 1H), 5.88 (s, 1H), 5.00 (s, 1H), 4.25 (d, J=4.6 Hz, 2H), 3.62-3.54 (m, 1H), 3.11-3.03 (m, 1H), 2.48-2.42 (m, 1H), 2.29 (s, 3H), 2.08-1.95 (m, 1H), 1.94-1.85 (m, 1H), 1.78-1.65 (m, 2H), 1.65-1.55 (m, 1H), 1.03 (d, J=6.3 Hz, 6H). LC-MS (ESI): m/z [M+H]⁺=434.2.

### Example 26: cis-3-(3-((7-methyl-1,1-dioxido-2,3-dihydrobenzo[d]isothiazol-5-yl)amino)-1H-pyrazol-5-yl)cyclopentyl isopropylcarbamate

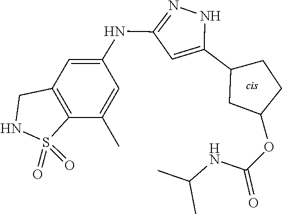 [SMILES: Cc1cc(Nc2cc(C3CCC(OC(=O)NC(C)C)C3)[nH]n2)cc2c1S(=O)(=O)NC2]

The titled compound was synthesized in the procedures similar to Example 1. ¹H NMR (500 MHz, DMSO-d₆) δ 11.82 (s, 1H), 8.82 (s, 1H), 7.37 (t, J=5 Hz, 1H), 7.19 (s, 1H), 7.03 (s, 1H), 6.87 (d, J=5 Hz, 1H), 5.62 (s, 1H), 4.97-4.89 (m, 1H), 4.17 (d, J=5 Hz, 2H), 3.56-3.47 (m, 1H), 3.03-2.93 (m, 1H), 2.41-2.35 (m, 1H), 2.31 (s, 3H), 1.99-1.91 (m, 1H), 1.88-1.78 (m, 1H), 1.71-1.59 (m, 2H), 1.57-1.48 (m, 1H), 1.01-0.90 (m, 6H). LC-MS (ESI): m/z [M+H]⁺=434.2.

### Example 27: cis-3-(3-((6-chloro-1,1-dioxido-2,3-dihydrobenzo[d]isothiazol-5-yl)amino)-1H-pyrazol-5-yl)cyclopentyl isopropylcarbamate

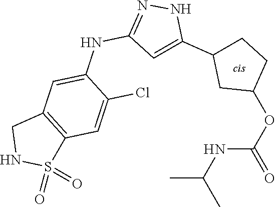 [SMILES: CC(C)NC(=O)OC1CCC(c2cc(Nc3cc4c(cc3Cl)S(=O)(=O)NC4)n[nH]2)C1]

The titled compound was synthesized in the procedures similar to Example 1. ¹H NMR (500 MHz, DMSO-d₆) δ 12.03 (s, 1H), 8.12 (s, 1H), 7.92 (s, 1H), 7.77 (s, 1H), 7.58 (t, J=5.1 Hz, 1H), 6.87 (d, J=7.7 Hz, 1H), 5.91 (s, 1H), 4.93 (s, 1H), 4.23 (d, J=5.3 Hz, 2H), 3.57-3.47 (m, 1H), 3.06-2.94 (m, 1H), 2.50-2.37 (m, 1H), 1.96-1.92 (m, 1H), 1.90-1.80 (m, 1H), 1.70-1.60 (m, 2H), 1.54-1.48 (m, 1H), 0.96 (d, J=6.3 Hz, 6H). LC-MS (ESI): m/z [M+H]⁺=454.2.

### Example 28: cis-3-(3-((1,1-dioxido-6-(trifluoromethoxy)-2,3-dihydrobenzo[d]isothiazol-5-yl)amino)-1H-pyrazol-5-yl)cyclopentyl isopropylcarbamate

### Step 1: tert-butyl(2-bromo-5-(trifluoromethoxy)phenyl)carbamate

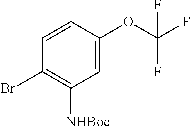 [SMILES: CC(C)(C)OC(=O)Nc1cc(OC(F)(F)F)ccc1Br]

To a solution of 2-bromo-5-(trifluoromethoxy) aniline (1.0 g, 3.9 mmol) in THF (20 mL), NaHMDS (9.77 mL, 9.77 mmol) was added at 0-5° C. Then Di-tert-butyl dicarbonate (1.02 g, 4.7 mmol) was added and the mixture was stirred at 25° C. for 12 hrs. The mixture was quenched with saturated NH₄Cl (aq.) (10 mL) then adjusted to PH=7-8 with 2N HCl. The mixture was extracted by EA (30 mL×3). The EA layer was concentrated under reduced pressure to afford the product, which was used for next step without purification. (1.01 g). LC-MS (ESI): m/z [M+H]⁺=356.4.

### Step 2: ethyl 2-((tert-butoxycarbonyl)amino)-4-(trifluoromethoxy)benzoate

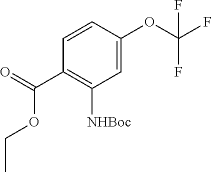 [SMILES: CCOC(=O)c1ccc(OC(F)(F)F)cc1NC(=O)OC(C)(C)C]

A mixture of tert-butyl(2-bromo-5-(trifluoromethoxy)phenyl)carbamate (1.42 g, 5.55 mmol), TEA (1.68 g, 16.64 mmol) and Pd(dppf)Cl₂ (0.4 g, 0.55 mmol) in EtOH (30 mL) was heated at under CO (gas, 4 Mpa) for 12 hrs. The mixture was concentrated under reduced pressure. The residue was purified by silica gel column chromatography, eluting with PE/EtOAc (9:1) to afford the product (0.6 g, 31.1%).

LC-MS (ESI): m/z [M+H]⁺=350.2

### Step 3: ethyl 2-amino-4-(trifluoromethoxy)benzoate

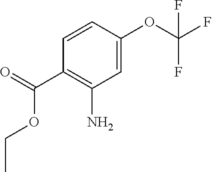 [SMILES: CCOC(=O)c1ccc(OC(F)(F)F)cc1N]

To a solution of ethyl 2-((tert-butoxycarbonyl)amino)-4-(trifluoromethoxy)benzoate (3.0 g, 8.6 mmol) in DCM (30 mL), TFA (30 mL) was added at 25° C. Then the mixture was stirred at 25° C. for 1 hr. The mixture was quenched with saturated NaHCO₃ (aq.) (20 mL). The mixture was extracted by DCM (30 mL×3). The DCM layer was concentrated under reduced pressure to afford the product, which was used for next step without purification. (2.02 g, 94.4%). LC-MS (ESI): m/z [M+H]⁺=250.1

### Step 4: ethyl 2-amino-5-bromo-4-(trifluoromethoxy)benzoate

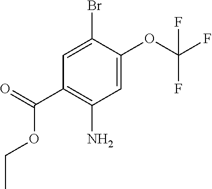 [SMILES: CCOC(=O)c1cc(Br)c(OC(F)(F)F)cc1N]

To a solution of ethyl 2-amino-4-(trifluoromethoxy)benzoate (2.02 g, 8.10 mmol) in DMF (30 mL), NBS (1.58 g, 8.91 mmol) was added at 0° C. Then the mixture was stirred at 0° C. for 1 hr. The mixture was concentrated under reduced pressure. The residue was purified by silica gel column chromatography, eluting with PE/EtOAc (10: 1~2:1) to afford the product (1.9 g, 71.7%). LC-MS (ESI): m/z [M+H]⁺=328.1

### Step 5: cis-3-(3-((1,1-dioxido-6-(trifluoromethoxy)-2,3-dihydrobenzo[d]isothiazol-5-yl)amino)-1H-pyrazol-5-yl)cyclopentyl isopropylcarbamate

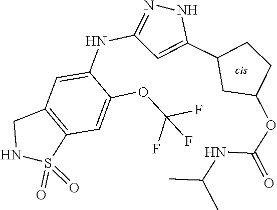 [SMILES: CC(C)NC(=O)OC1CCC(c2cc(Nc3cc4c(cc3OC(F)(F)F)S(=O)(=O)NC4)n[nH]2)C1]

The titled compound was synthesized in the procedures similar to Example 1. ¹H NMR (500 MHz, DMSO-d₆)) δ 12.07 (s, 1H), 8.74 (s, 1H), 8.15 (s, 1H), 7.76-7.67 (m, 2H), 7.09-6.78 (m, 1H), 5.92 (s, 1H), 5.01 (s, 1H), 4.33 (d, J=5.2 Hz, 2H), 3.62-3.55 (m, 1H), 3.15-3.03 (m, 1H), 2.48-2.42 (m, 1H), 2.10-2.01 (m, 1H), 1.95-1.86 (m, 1H), 1.78-1.65 (m, 2H), 1.65-1.55 (m, 1H), 1.03 (d, J=5.9 Hz, 6H). LC-MS (ESI): m/z [M+H]⁺=504.2.

### Example 29: cis-3-(3-((7-fluoro-1,1-dioxido-2,3-dihydrobenzo[d]isothiazol-5-yl)amino)-1H-pyrazol-5-yl)cyclopentyl isopropylcarbamate

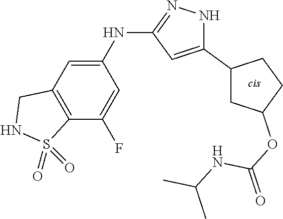 [SMILES: CC(C)NC(=O)OC1CCC(c2cc(Nc3cc(F)c4c(c3)CNS4(=O)=O)n[nH]2)C1]

The titled compound was synthesized in the procedures similar to Example 1. ¹H NMR (500 MHz, DMSO-d₆) δ 12.01 (s, 1H), 9.28 (s, 1H), 7.71 (s, 1H), 7.29 (d, J=10 Hz, 1H), 7.11 (s, 1H), 6.94 (d, J=5 Hz, 1H), 5.72 (s, 1H), 5.04-4.96 (m, 1H), 4.32 (s, 2H), 3.62-3.55 (m, 1H), 3.12-3.02 (m, 1H), 2.48-2.43 (m, 1H), 2.06-1.98 (m, 1H), 1.96-1.85 (m, 1H), 1.78-1.67 (m, 2H), 1.65-1.55 (m, 1H), 1.08-0.97 (m, 6H). LC-MS (ESI): m/z [M+H]⁺=438.2.

### Example 30: cis-3-(3-((4,6-difluoro-1,1-dioxido-2,3-dihydrobenzo[d]isothiazol-5-yl)amino)-1H-pyrazol-5-yl)cyclopentyl isopropylcarbamate

### Step 1: methyl 6-amino-3-bromo-2,4-difluorobenzoate

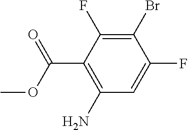

The titled compound was synthesized in the procedures similar to Example 20, step 1. LC-MS (ESI): m/z [M+H]⁺=266.1

### Step 2: cis-3-(3-((4,6-difluoro-1,1-dioxido-2,3-dihydrobenzo[d]isothiazol-5-yl)amino)-1H-pyrazol-5-yl)cyclopentyl isopropylcarbamate

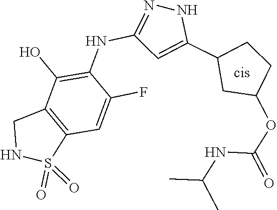 [SMILES: CC(C)NC(=O)OC1CCC(c2cc(Nc3c(F)cc4c(c3O)CNS4(=O)=O)n[nH]2)C1]

The titled compound was synthesized in the procedures similar to Example 1. ¹H NMR (500 MHz, DMSO-d₆) δ=11.81 (s, 1H), 8.13 (s, 1H), 7.93 (t, J=5.0, 1H), 7.66 (d, J=8.6, 1H), 6.94 (d, J=7.4, 1H), 5.63 (s, 1H), 4.98 (s, 1H), 4.37 (d, J=5.3, 2H), 3.57 (m, 1H), 3.07-2.95 (m, 1H), 2.47-2.39 (m, 1H), 1.99 (m, 1H), 1.95-1.84 (m, 1H), 1.69 (m, 2H), 1.57 (s, 1H), 1.03 (m, 6H). LC-MS (ESI): m/z [M+H]⁺=456.2.

### Example 31: cis-3-(3-((1,1-dioxido-2,3-dihydroisothiazolo[5,4-b]pyridin-5-yl)amino)-1H-pyrazol-5-yl)cyclopentyl isopropylcarbamate

### Step 1:2-(benzylthio)-5-bromo-3-methylpyridine

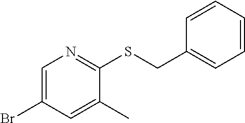 [SMILES: Cc1cc(Br)cnc1SCc1ccccc1]

A mixture of phenylmethanethiol (1.2 g. 9.69 mmol) in THF (50 mL) was added NaH (60% dispersion in mineral oil, 465 mg, 11.63 mmol) one portion, stirred for 30 minutes at room temperature, 5-bromo-2-chloro-3-methylpyridine (2.0 g, 9.69 mmol) in THF (10 mL) was added dropwise over 5 minutes, the reaction mixture was stirred for 16 hours at room temperature. The resulting mixture was quenched with saturate NH₄Cl aqueous, extracted with EtOAc (3×50 mL), the combined organic phase was washed with brine, dried over Na₂SO₄, filtered, concentrated under reduce pressure, dried over in vacuo, to afford the crude product. (2.95 g crude). LC-MS (ESI): m/z [M+H]⁺=294.1.

### Step 2:5-bromo-N-(tert-butyl)-3-methylpyridine-2-sulfonamide

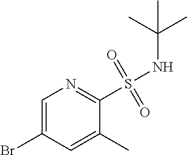 [SMILES: Cc1cc(Br)cnc1S(=O)(=O)NC(C)(C)C]

2-(benzylthio)-5-bromo-3-methylpyridine (300 mg, 1.02 mmol) was dissolved in DCM (15 mL), SO₂Cl₂ (700 mg, 5.1 mmol) was added, stirred for 30 minutes, quenched with water, the organic phase was separated, washed with brine, dried over Na₂SO₄, filtered, concentrated under reduce pressure, dried over in vacuo to give a solid. The solid was redissolved in DCM (15 mL), t-BuNH₂ (0.5 mmol) was added, stirred another 16 hours at room temperature. The resulting mixture was concentrated, purified by silica gel column chromatography, eluting with PE/EA (3:1) to afford the product (205 mg, 66%). LC-MS (ESI): m/z [M+H]⁺=307.3.

### Step 3:5-bromo-3-(bromomethyl)-N-(tert-butyl)pyridine-2-sulfonamide

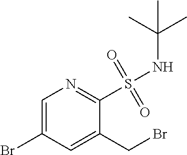 [SMILES: CC(C)(C)NS(=O)(=O)c1ncc(Br)cc1CBr]

To a solution of 5-bromo-N-(tert-butyl)-3-methylpyridine-2-sulfonamide (800 mg, 2.61 mmol) in CCl₄ (50 mL) was added NBS (930 mg, 5.23 mmol) and AIBN (43 mg, 0.26 mmol), the reaction mixture was stirred for 16 hours under refluxed. The resulting mixture was cooled to room temperature, filtered, the filtrate was concentrated, dried over in vacuo, to afford the crude product (1.2 g crude). LC-MS (ESI): m/z [M+H]⁺=385.1.

### Step 4:5-bromo-2-(tert-butyl)-2,3-dihydroisothiazolo[5,4-b]pyridine 1,1-dioxide

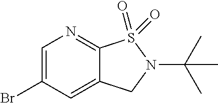 [SMILES: CC(C)(C)N1Cc2cc(Br)cnc2S1(=O)=O]

5-bromo-3-(bromomethyl)-N-(tert-butyl)pyridine-2-sulfonamide (1.2 g crude) was dissolved in THF (50 mL), NaH (60% dispersion in mineral oil, 200 mg, 5 mmol) was added one portion, the reaction was stirred for 4 hours at room temperature. The resulting mixture was quenched with water, extracted with EA (3×50 mL), the combined organic phase was washed with brine, dried over Na₂SO₄, filtered, concentrated under reduce pressure, dried over in vacuo, recrystallized from PE/EA (10:1) to afford the product. (153 mg, 19% for 2 steps). LC-MS (ESI): m/z [M+H]⁺=305.1.

### Step 5: cis-3-(1-(tert-butyl)-5-((2-(tert-butyl)-1,1-dioxido-2,3-dihydroisothiazolo[5,4-b]pyridin-5-yl)amino)-1H-pyrazol-3-yl)cyclopentyl isopropylcarbamate

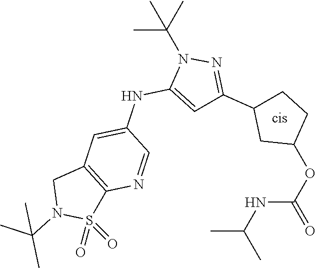 [SMILES: CC(C)NC(=O)OC1CCC(c2cc(Nc3cnc4c(c3)CN(C(C)(C)C)S4(=O)=O)n(C(C)(C)C)n2)C1]

To a mixture of 5-bromo-2-(tert-butyl)-2,3-dihydroisothiazolo[5,4-b]pyridine 1,1-dioxide (50 mg, 0.164 mmol) and cis-3-(5-amino-1-(tert-butyl)-1H-pyrazol-3-yl)cyclopentyl isopropylcarbamate (505 mg, 0.164 mmol) in andydrous 1,4-dioxane (5 mL) was added Pd₂(dba)₃ (15 mg, 0.016 mmol), XantPhos (19 mg, 0.032 mmol) and K₃PO₄ (104 mg, 0.492 mmol). The reaction mixture was stirred at 95° C. under nitrogen atmosphere for 16 h. The mixture was cooled to room temperature, filtered, and the filtrate was concentrated under reduced pressure. The residue was purified by silica gel column chromatography, eluting with PE/EA (2:1) to afford the product (63 mg, 72%). LC-MS (ESI): m/z [M+H]⁺=533.4.

### Step 6: cis-3-(3-((1,1-dioxido-2,3-dihydroisothiazolo[5,4-b]pyridin-5-yl)amino)-1H-pyrazol-5-yl)cyclopentyl isopropylcarbamate

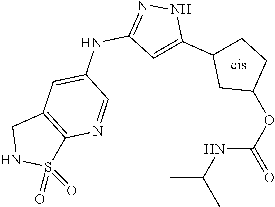 [SMILES: CC(C)NC(=O)OC1CCC(c2cc(Nc3cnc4c(c3)CNS4(=O)=O)n[nH]2)C1]

cis-3-(1-(tert-butyl)-5-((2-(tert-butyl)-1,1-dioxido-2,3-dihydroisothiazolo[5,4-b]pyridin-5-yl)amino)-1H-pyrazol-3-yl)cyclopentyl isopropylcarbamate (63 mg, 0.12 mmol) was dissolved in formic acid (5 mL). The reaction mixture was stirred for 16 hours at 80° C. The resulting mixture was cooled to room temperature, concentrated under reduce pressure, redissolved in DCM (5 mL), 2 N HCl aqueous (1 mL) was added, stirred for 2 hours at room temperature. The resulting mixture was concentrated under reduce pressure, purified by prep-HPLC (Waters X-select Prep C18 OBD 19×150 mm, 5 μm, eluting with 27%-42% of acetonitrile (containing 0.1% FA) in water (containing 0.1% FA) to afford the product (10 mg, 20%). ¹H NMR (500 MHz, DMSO-d₆) δ=12.08 (s, 1H), 9.42 (s, 1H), 8.56 (d, J=2.0, 1H), 8.07 (s, 1H), 7.73 (t, J=4.6, 1H), 7.01 (d, J=7.4, 1H), 5.80 (s, 1H), 5.06 (s, 1H), 4.40 (d, J=4.8, 2H), 3.64 (m, 6.4, 1H), 3.19-3.06 (m, 1H), 2.51 (m, 1H), 2.09 (m, 1H), 2.02-1.91 (m, 1H), 1.79 (m, 2H), 1.68 (m, 1H), 1.09 (m, 6H). LC-MS (ESI): m/z [M+H]⁺=421.2.

### Example 32: cis-3-(3-((2-methyl-1,1-dioxido-2,3-dihydroisothiazolo[5,4-b]pyridin-5-yl)amino)-1H-pyrazol-5-yl)cyclopentyl isopropylcarbamate

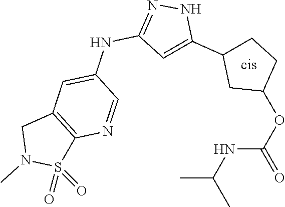 [SMILES: CC(C)NC(=O)OC1CCC(c2cc(Nc3cnc4c(c3)CN(C)S4(=O)=O)n[nH]2)C1]

The titled compound was synthesized in the procedures similar to Example 31. ¹H NMR (500 MHz, DMSO-d₆) δ=12.03 (s, 1H), 9.41 (s, 1H), 8.51 (s, 1H), 8.05 (s, 1H), 6.95 (d, J=7.5, 1H), 5.74 (s, 1H), 5.00 (s, 1H), 4.33 (s, 2H), 3.58 (m, 1H), 3.14-3.02 (m, 1H), 2.79 (s, 3H), 2.48-2.44 (m, 1H), 2.03 (m, 1H), 1.91 (m, 1H), 1.73 (m, 2H), 1.61 (m, 1H), 1.03 (m, 6H). LC-MS (ESI): m/z [M+H]⁺=435.3.

### Example 33: cis-3-(3-((1,1-dioxido-2,3-dihydroisothiazolo[4,5-b]pyridin-5-yl)amino)-1H-pyrazol-5-yl)cyclopentyl isopropylcarbamate

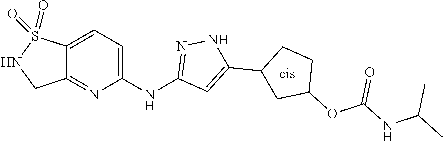 [SMILES: CC(C)NC(=O)OC1CCC(c2cc(Nc3ccc4c(n3)CNS4(=O)=O)n[nH]2)C1]

The titled compound was synthesized in the procedures similar to Example 31. ¹H NMR (500 MHz, DMSO-d₆) δ 12.08 (s, 1H), 10.03 (s, 1H), 7.95 (d, J=8.8 Hz, 1H), 7.64 (t, J=5.0 Hz, 1H), 7.24 (s, 1H), 6.94 (d, J=7.5 Hz, 1H), 6.21 (s, 1H), 5.10-4.92 (m, 1H), 4.27 (d, J=4.8 Hz, 2H), 3.68-3.52 (m, 1H), 3.13-3.02 (m, 1H), 2.48-2.42 (m, 1H), 2.07-1.97 (m, 1H), 1.96-1.86 (m, 1H), 1.80-1.69 (m, 2H), 1.66-1.56 (m, 1H), 1.03 (d, J=6.0 Hz, 6H. LC-MS (ESI): m/z [M+H]⁺=421.3.

### Example 34: cis-3-(3-((4-methyl-1,1-dioxido-2,3-dihydrobenzo[d]isothiazol-5-yl)amino)-1H-pyrazol-5-yl)cyclopentyl isopropylcarbamate

### Step 1:5-bromo-4-methyl-2,3-dihydrobenzo[d]isothiazole 1,1-dioxide

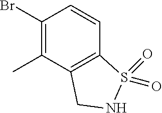

The titled compound was synthesized in the procedures similar to Example 1, step 10 to step 12.

LC-MS (ESI): m/z [M+H]⁺=262.3.

### Step 2: cis-benzyl(5-(3-(((4-nitrophenoxy) carbonyl)oxy) cyclopentyl)-1H-pyrazol-3-yl)carbamate

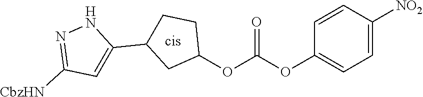 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl(1-(tert-butyl)-3-(3-(((4-nitrophenoxy) carbonyl)oxy) cyclopentyl)-1H-pyrazol-5-yl)carbamate (30 g, 57.5 mmol) was added 100 mL formic acid. The resulting mixture was stirred at 75° C. for over night. The solvent was removed under vacuum to yield 26 g of crude product, which was directly used for the next step without further purification.

### Step 3: cis-3-(3-(((benzyloxy) carbonyl)amino)-1H-pyrazol-5-yl)cyclopentyl isopropylcarbamate

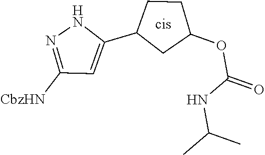 [SMILES: CC(C)NC(=O)OC1CCC(c2cc(NC(=O)OCc3ccccc3)n[nH]2)C1]

To a solution of cis-benzyl(5-(3-(((4-nitrophenoxy) carbonyl)oxy) cyclopentyl)-1H-pyrazol-3-yl)carbamate (7 g, 15 mmol) in 100 mL THF was added propan-2-amine (2.6 g, 45 mmol). The resulting mixture was stirred at room temperature for 1 h. The resulting mixture was concentrated under reduced pressure, and the residue was purified by silica gel chromatography (PE/EA=2:1) to afford the product (4.6 g, 80% yield). LC-MS (ESI): m/z [M+H]⁺=387.3.

### Step 4: cis-3-(3-amino-1H-pyrazol-5-yl)cyclopentyl isopropylcarbamate

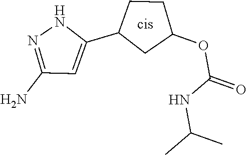 [SMILES: CC(C)NC(=O)OC1CCC(c2cc(N)n[nH]2)C1]

To a solution of cis-3-(3-(((benzyloxy) carbonyl)amino)-1H-pyrazol-5-yl)cyclopentyl isopropylcarbamate (4.6 g, 12 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (2.5 g, 82% yield).

LC-MS (ESI): m/z [M+H]⁺=253.4.

### Step 5: cis-3-(3-((4-methyl-1,1-dioxido-2,3-dihydrobenzo[d]isothiazol-5-yl)amino)-1H-pyrazol-5-yl)cyclopentyl isopropylcarbamate

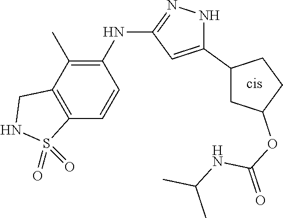 [SMILES: Cc1c(Nc2cc(C3CCC(OC(=O)NC(C)C)C3)[nH]n2)ccc2c1CNS2(=O)=O]

A mixture of 5-bromo-4-methyl-2,3-dihydrobenzo[d]isothiazole 1,1-dioxide (90 mg, 0.33 mmol), cis-3-(3-amino-1H-pyrazol-5-yl)cyclopentyl isopropylcarbamate (65.5 mg, 0.26 mmol), Brettphos Pd G3 (29.9 mg, 0.033 mmol) and K₂CO₃ (135 mg, 0.98 mmol) in t-BuOH (5 mL) was stirred at 110° C. for 16 h under a nitrogen atmosphere. LCMS showed the reaction was complete. The mixture was filtered and the filtrate was concentrated under vacuum. The residue was purified by silica gel column chromatography, eluting with PE/EA (0-100%) to afford the product (51.9 mg, 31.6%). ¹H NMR (500 MHz, DMSO-d₆) δ 11.96 (s, 1H), 7.90-7.80 (brs, 1H), 7.77 (s, 1H), 7.51 (t, J=5 Hz, 1H), 7.43 (d, J=10 Hz, 1H), 6.97-6.91 (m, 1H), 5.84 (s, 1H), 5.04-4.96 (m, 1H), 4.32-4.25 (m, 2H), 3.63-3.53 (m, 1H), 3.12-3.02 (m, 1H), 2.46-2.4 (m, 1H), 2.12 (s, 3H), 2.06-1.99 (m, 1H), 1.95-1.86 (m, 1H), 1.78-1.66 (m, 2H), 1.65-1.57 (m, 1H), 1.07-1.00 (m, 6H). LC-MS (ESI): m/z [M+H]⁺=434.2.

### Example 35: cis-3-(3-((2,4-dimethyl-1,1-dioxido-2,3-dihydrobenzo[d]isothiazol-5-yl)amino)-1H-pyrazol-5-yl)cyclopentyl isopropylcarbamate

### Step 1:5-bromo-2,4-dimethyl-2,3-dihydrobenzo[d]isothiazole 1,1-dioxide

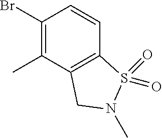

To a mixture of 5-bromo-4-methyl-2,3-dihydrobenzo[d]isothiazole 1,1-dioxide (170 mg, 0.65 mmol) and Cs₂CO₃ (255 mg, 0.78 mmol) in DMF (10 mL) was added iodomethane (102 mg, 0.71 mmol). The mixture was stirred at 50° C. for 2 h. LCMS showed the reaction was complete. The mixture was diluted with water (50 mL), extracted with EA (3×50 mL). The combined organic layers were dried over

Na₂SO₄, filtered and concentrated under vacuum. The residue was purified by silica gel column chromatography, eluting with PE/EA (3:2) to afford the product (90 mg, 50%). LC-MS (ESI): m/z [M+H]⁺=276.2.

### Step 2: cis-3-(3-((2,4-dimethyl-1,1-dioxido-2,3-dihydrobenzo[d]isothiazol-5-yl)amino)-1H-pyrazol-5-yl)cyclopentyl isopropylcarbamate

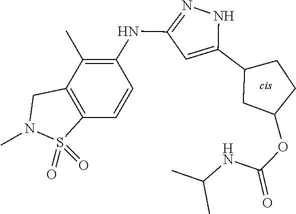 [SMILES: Cc1c(Nc2cc(C3CCC(OC(=O)NC(C)C)C3)[nH]n2)ccc2c1CN(C)S2(=O)=O]

The titled compound was synthesized in the procedures similar to Example 34, step 5. ¹H NMR (500 MHz, DMSO-d₆) δ 11.98 (s, 1H), 7.92-7.84 (brs, 1H), 7.82 (s, 1H), 7.48 (t, J=10 Hz, 1H), 6.96-6.91 (m, 1H), 5.04-4.95 (m, 1H), 4.29 (s, 2H), 3.62-3.53 (m, 1H), 3.13-3.02 (m, 1H), 2.78 (s, 3H), 2.48-2.43 (m, 1H), 2.12 (s, 3H), 2.06-1.98 (m, 1H), 1.96-1.66 (m, 1H), 1.78-1.66 (m, 2H), 1.65-1.55 (m, 1H), 1.09-0.96 (m, 6H). LC-MS (ESI): m/z [M+H]⁺=448.2.

### Example 36: cis-3-(3-((2-(2-methoxyethyl)-4-methyl-1,1-dioxido-2,3-dihydrobenzo[d]isothiazol-5-yl)amino)-1H-pyrazol-5-yl)cyclopentyl isopropylcarbamate

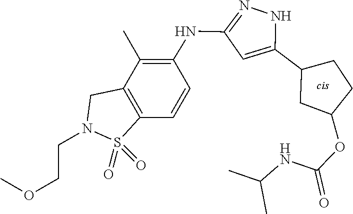 [SMILES: COCCN1Cc2c(ccc(Nc3cc(C4CCC(OC(=O)NC(C)C)C4)[nH]n3)c2C)S1(=O)=O]

The titled compound was synthesized in the procedures similar to Example 35. ¹H NMR (500 MHz, DMSO-d₆) δ 11.98 (s, 1H), 7.89 (t, J=5 Hz, 1H), 7.82 (s, 1H), 7.47 (d, J=10 Hz, 1H), 6.98-6.90 (m, 1H), 5.85 (s, 1H), 5.05-4.96 (m, 1H), 4.39 (s, 2H), 3.62 (t, J=5 Hz, 2H), 3.60-3.52 (m, 1H), 3.30 (s, 3H), 3.11-3.03 (m, 1H), 2.49-2.42 (m, 3H), 2.12 (s, 3H), 2.07-1.99 (m, 1H), 1.96-1.85 (m, 1H), 1.76-1.66 (m, 2H), 1.64-1.56 (m, 1H), 1.08-0.95 (m, 6H). LC-MS (ESI): m/z [M+H]⁺=492.2.

### Example 37: cis-3-(3-((2-(2-hydroxyethyl)-4-methyl-1,1-dioxido-2,3-dihydrobenzo[d]isothiazol-5-yl)amino)-1H-pyrazol-5-yl)cyclopentyl isopropylcarbamate

### Step 1: cis-3-(3-((2-(2-((tert-butyldimethylsilyl)oxy) ethyl)-4-methyl-1,1-dioxido-2,3-dihydrobenzo[d]isothiazol-5-yl)amino)-1H-pyrazol-5-yl)cyclopentyl isopropylcarbamate

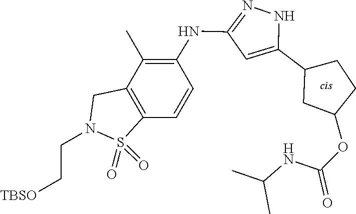 [SMILES: Cc1c(Nc2cc(C3CCC(OC(=O)NC(C)C)C3)[nH]n2)ccc2c1CN(CCO[Si](C)(C)C(C)(C)C)S2(=O)=O]

The titled compound was synthesized in the procedures similar to Example 36. LC-MS (ESI): m/z [M+H]⁺=592.4.

### Step 2: cis-3-(3-((2-(2-hydroxyethyl)-4-methyl-1,1-dioxido-2,3-dihydrobenzo[d]isothiazol-5-yl)amino-1H-pyrazol-5-yl)cyclopentyl isopropylcarbamate

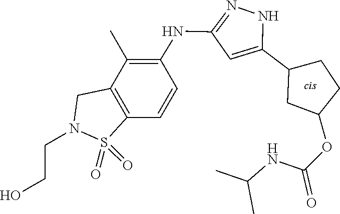 [SMILES: Cc1c(Nc2cc(C3CCC(OC(=O)NC(C)C)C3)[nH]n2)ccc2c1CN(CCO)S2(=O)=O]

To a flask charged with cis-3-(3-((2-(2-((tert-butyldimethylsilyl)oxy) ethyl)-4-methyl-1,1-dioxido-2,3-dihydrobenzo[d]isothiazol-5-yl)amino)-1H-pyrazol-5-yl)cyclopentyl isopropylcarbamate (101 mg, 0.17 mmol) was added TFA (1 mL) and DCM (3 mL). The result solution was stirred at room temperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (59 mg, 12%). ¹H NMR (500 MHz, DMSO-d₆) δ 11.98 (s, 1H), 7.89 (s, 1H), 7.82 (s, 1H), 7.47 (d, J=8.7 Hz, 1H), 6.94 (d, J=7.2 Hz, 1H), 5.85 (s, 1H), 5.00 (s, 1H), 4.40 (s, 2H), 3.69 (t, J=6.0 Hz, 2H), 3.58 (dd, J=13.1, 6.3 Hz, 1H), 3.20 (t, J=6.0 Hz, 2H), 3.14-3.01 (m, 1H), 2.49-2.43 (m, 1H), 2.13 (s, 3H), 2.03 (dd, J=15.5, 7.8 Hz, 1H), 1.91 (dt, J=16.2, 8.0 Hz, 1H), 1.80-1.66 (m, 2H), 1.65-1.52 (m, 1H), 1.02 (d, J=6.1 Hz, 6H). LC-MS (ESI): m/z [M+H]⁺=492.2.

### Example 38: cis-3-(3-((4-chloro-1,1-dioxido-2,3-dihydrobenzo[d]isothiazol-5-yl)amino)-1H-pyrazol-5-yl)cyclopentyl isopropylcarbamate

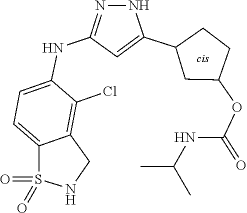 [SMILES: CC(C)NC(=O)OC1CCC(c2cc(Nc3ccc4c(c3Cl)CNS4(=O)=O)n[nH]2)C1]

The titled compound was synthesized in the procedures similar to Example 1 in racemic form, which was further separated by Chiral Prep-HPLC to give:

Enantiomer 1 (Example 38a, 100% ee); Retention time: 4.59 min. ¹H NMR (500 MHz, DMSO-d₆) δ 12.15 (brs, 1H), 8.25 (s, 1H), 8.05 (d, J=7.7 Hz, 1H), 7.74 (s, 1H), 7.61 (d, J=8.7 Hz, 1H), 6.94 (d, J=6.9 Hz, 1H), 5.96 (s, 1H), 5.06-4.93 (m, 1H), 4.30 (s, 2H), 3.65-3.50 (m, 1H), 3.23-2.98 (m, 1H), 2.48-2.40 (m, 1H), 2.10-1.99 (m, 1H), 1.98-1.86 (m, 1H), 1.80-1.66 (m, 2H), 1.65-1.55 (m, 1H), 1.03 (d, J=6.1 Hz, 6H). LC-MS (ESI): m/z [M+H]⁺=454.27.

Enantiomer 2 (Example 38b, 100% ee); Retention time: 7.29 min. ¹H NMR (500 MHz, DMSO-d₆) δ 12.10 (brs, 1H), 8.25 (s, 1H), 8.05 (d, J=7.8 Hz, 1H), 7.74 (s, 1H), 7.61 (d, J=8.7 Hz, 1H), 5.96 (s, 1H), 5.07-4.93 (m, 1H), 4.30 (s, 2H), 3.65-3.41 (m, 1H), 3.24-2.98 (m, 1H), 2.49-2.40 (m, 1H), 2.10-1.99 (m, 1H), 1.98-1.85 (m, 1H), 1.80-1.66 (m, 2H), 1.65-1.55 (m, 1H), 1.10-0.97 (m, 6H). LC-MS (ESI): m/z [M+H]⁺=454.27.

Chiral analytical method: Column: CHIRALPAK IF 4.6 mm×150 mm, 5 μm; Mobile phase: A for Hexane and B for EtOH (0.1% 2M NH₃ MeOH); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IF 20 mm×250 mm, 5 μm; Mobile phase: A for Hexane and B for EtOH (0.2% 2M NH₃ MeOH); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 39: cis-3-(3-((4-chloro-1,1-dioxido-2,3-dihydrobenzo[d]isothiazol-5-yl)amino)-1H-pyrazol-5-yl)cyclopentyl propylcarbamate

### Step 1: cis-3-(3-amino-1H-pyrazol-5-yl)cyclopentyl propylcarbamate

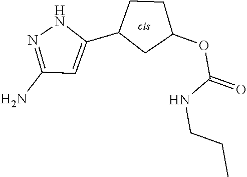 [SMILES: CCCNC(=O)OC1CCC(c2cc(N)n[nH]2)C1]

The titled compound was synthesized in the procedures similar to Example 34, step 3 to step 4.

LC-MS (ESI): m/z [M+H]⁺=253.2.

### Step 2: cis-3-(3-((4-chloro-1,1-dioxido-2,3-dihydrobenzo[d]isothiazol-5-yl)amino)-1H-pyrazol-5-yl)cyclopentyl propylcarbamate

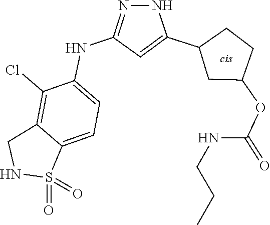 [SMILES: CCCNC(=O)OC1CCC(c2cc(Nc3ccc4c(c3Cl)CNS4(=O)=O)n[nH]2)C1]

The titled compound was synthesized in the procedures similar to Example 34, step 5 in a racemic form, which was further separated by Chiral Prep-HPLC to give:

Enantiomer 1 (Example 39a, 100% ee); Retention time: 4.32 min. ¹H NMR (500 MHz, DMSO-d₆) δ 12.12 (s, 1H), 8.24 (s, 1H), 8.07 (s, 1H), 7.73 (s, 1H), 7.61 (d, J=8.7 Hz, 1H), 7.04 (t, J=5.4 Hz, 1H), 5.96 (s, 1H), 5.08-4.95 (m, 1H), 4.30 (d, J=3.7 Hz, 2H), 3.14-3.04 (m, 1H), 2.95-2.85 (m, 2H), 2.48-2.43 (m, 1H), 2.08-2.01 (m, 1H), 1.96-1.86 (m, 1H), 1.80-1.68 (m, 2H), 1.66-1.57 (m, 1H), 1.43-1.31 (m, 2H), 0.81 (t, J=7.4 Hz, 3H). LC-MS (ESI): m/z [M+H]⁺=454.20.

Enantiomer 2 (Example 39b, 100% ee); Retention time: 5.73 min. ¹H NMR (500 MHz, DMSO-d₆) δ 12.12 (s, 1H), 8.24 (s, 1H), 8.07 (s, 1H), 7.73 (s, 1H), 7.61 (d, J=8.7 Hz, 1H), 7.04 (t, J=5.4 Hz, 1H), 5.96 (s, 1H), 5.08-4.95 (m, 1H), 4.30 (d, J=3.7 Hz, 2H), 3.14-3.04 (m, 1H), 2.95-2.85 (m, 2H), 2.48-2.43 (m, 1H), 2.08-2.01 (m, 1H), 1.96-1.86 (m, 1H), 1.80-1.68 (m, 2H), 1.66-1.57 (m, 1H), 1.43-1.31 (m, 2H), 0.81 (t, J=7.4 Hz, 3H). LC-MS (ESI): m/z [M+H]⁺=454.20.

Chiral analytical method: Column: CHIRALPAK IE 4.6 mm×250 mm, 5 μm; Mobile phase: A for MtBE (0.1% 2M NH₃ MeOH) (%) and B for MeOH/DCM (50:50) (%); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for MeOH/DCM (50:50) (%); Gradient: Mobile Phase A: Mobile Phase B=20: 80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 40: cis-3-(3-((4-chloro-1,1-dioxido-2,3-dihydrobenzo[d]isothiazol-5-yl)amino)-1H-pyrazol-5-yl)cyclopentyl(1-methylcyclopropyl)carbamate

### Step 1: cis-3-(3-(((benzyloxy) carbonyl)amino)-1H-pyrazol-5-yl)cyclopentyl(1-methylcyclopropyl)carbamate

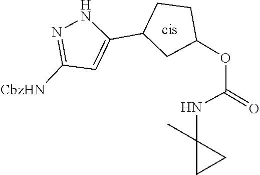 [SMILES: CC1(NC(=O)OC2CCC(c3cc(NC(=O)OCc4ccccc4)n[nH]3)C2)CC1]

To a solution of cis-benzyl(5-(3-(((4-nitrophenoxy) carbonyl)oxy) cyclopentyl)-1H-pyrazol-3-yl)carbamate (10 g, 21.5 mmol) in 100 mL THF was added 1-Methylcyclopropylamine hydrochloride (11.5 g, 107 mmol) and DIEA (27.6 g, 214 mmol). The resulting mixture was stirred at room temperature for 12 h. The resulting mixture was concentrated under reduced pressure and dissolved in EA (200 mL). The mixture was washed with water (200 mL×3), dried with Na₂SO₄, and concentrated. The residue was triturated with MTBE and filtered to afford the product (3.6 g, 42% yield). LC-MS (ESI): m/z [M+H]⁺=399.3.

### Step 2: cis-3-(3-amino-1H-pyrazol-5-yl)cyclopentyl(1-methylcyclopropyl)carbamate

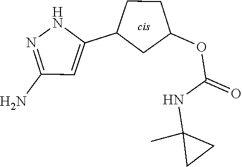 [SMILES: CC1(NC(=O)OC2CCC(c3cc(N)n[nH]3)C2)CC1]

To a solution of cis-3-(3-(((benzyloxy) carbonyl)amino)-1H-pyrazol-5-yl)cyclopentyl(1-methylcyclopropyl)carbamate (3.6 g, 9 mmol) in THF (100 mL) was added Pd/C (10%, wet, 2 g). The suspension was degassed and purged with hydrogen 3 times. The resulting mixture was stirred at room temperature under a hydrogen balloon for 3 h. The mixture was filtered, and the filter cake was washed with EA (200 mL×3). The filtrate was concentrated under reduced pressure to afford the product (2.2 g, 92% yield). LC-MS (ESI): m/z [M+H]⁺=265.3.

### Step 3: cis-3-(3-((4-chloro-1,1-dioxido-2,3-dihydrobenzo[d]isothiazol-5-yl)amino)-1H-pyrazol-5-yl)cyclopentyl(1-methylcyclopropyl)carbamate

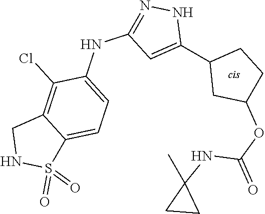 [SMILES: CC1(NC(=O)OC2CCC(c3cc(Nc4ccc5c(c4Cl)CNS5(=O)=O)n[nH]3)C2)CC1]

The titled compound was synthesized in the procedures similar to Example 34, step 5 in a racemic form, which was further separated by Chiral Prep-HPLC to get:

Enantiomer 1 (Example 40a, 100% ee); Retention time: 4.40 min. ¹H NMR (500 MHz, DMSO-d₆) δ 12.11 (brs, 1H), 8.24 (s, 1H), 8.04 (s, 1H), 7.73 (s, 1H), 7.61 (d, J=8.7 Hz, 1H), 7.34 (s, 1H), 5.95 (s, 1H), 5.10-4.90 (m, 1H), 4.30 (s, 2H), 3.18-3.01 (m, 1H), 2.57-2.53 (m, 1H), 2.08-1.97 (m, 1H), 1.96-1.85 (m, 1H), 1.78-1.64 (m, 2H), 1.63-1.52 (m, 1H), 1.23 (s, 3H), 0.65-0.53 (m, 2H), 0.0.52-0.45 (m, 2H).

LC-MS (ESI): m/z [M+H]⁺=466.30.

Enantiomer 2 (Example 40b, 100% ee); Retention time: 5.64 min. ¹H NMR (500 MHz, DMSO-d₆) δ 12.11 (brs, 1H), 8.24 (s, 1H), 8.04 (s, 1H), 7.73 (s, 1H), 7.61 (d, J=8.7 Hz, 1H), 7.34 (s, 1H), 5.95 (s, 1H), 5.10-4.90 (m, 1H), 4.30 (s, 2H), 3.18-3.01 (m, 1H), 2.57-2.53 (m, 1H), 2.08-1.97 (m, 1H), 1.96-1.85 (m, 1H), 1.78-1.64 (m, 2H), 1.63-1.52 (m, 1H), 1.23 (s, 3H), 0.65-0.53 (m, 2H), 0.0.52-0.45 (m, 2H).

LC-MS (ESI): m/z [M+H]⁺=466.30.

Chiral analytical method: Column: CHIRALPAK IE 4.6 mm×250 mm, 5 μm; Mobile phase: A for MtBE (0.1% 2M NH₃ MeOH) (%) and B for MeOH/DCM (50:50) (%); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for MeOH/DCM (50:50) (%); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 41: cis-3-(3-((4-chloro-1,1-dioxido-2,3-dihydrobenzo[d]isothiazol-5-yl)amino)-1H-pyrazol-5-yl)cyclopentyl((S)-sec-butyl)carbamate

### Step 1: cis-3-(3-amino-1H-pyrazol-5-yl)cyclopentyl((S)-sec-butyl)carbamate

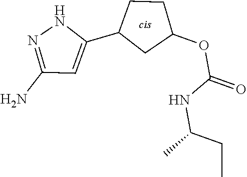 [SMILES: CC[C@H](C)NC(=O)OC1CCC(c2cc(N)n[nH]2)C1]

The titled compound was synthesized in the procedures similar to Example 34, step 3 to step 4.

LC-MS (ESI): m/z [M+H]⁺=267.3.

### Step 2: cis-3-(3-((4-chloro-1,1-dioxido-2,3-dihydrobenzo[d]isothiazol-5-yl)amino)-1H-pyrazol-5-yl)cyclopentyl((S)-sec-butyl)carbamate

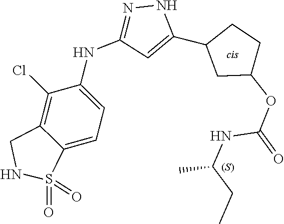

The titled compound was synthesized in the procedures similar to Example 34, step 3 to step 4. ¹H NMR (500 MHz, DMSO-d₆) δ 12.17 (brs, 1H), 8.24 (d, J=4.5 Hz, 1H), 8.04 (d, J=7.3 Hz, 1H), 7.73 (s, 1H), 7.61 (d, J=8.7 Hz, 1H), 6.88 (d, J=8.1 Hz, 1H), 5.96 (s, 1H), 5.10-4.90 (m, 1H), 4.30 (s, 2H), 3.43-3.26 (m, 1H), 3.17-2.96 (m, 1H), 2.48-2.42 (m, 1H), 2.10-1.99 (m, 1H), 1.98-1.86 (m, 1H), 1.80-1.67 (m, 2H), 1.66-1.55 (m, 1H), 1.43-1.27 (m, 2H), 1.10-0.93 (m, 3H), 0.89-0.70 (m, 3H). LC-MS (ESI): m/z [M+H]⁺=468.30.

### Example 42: cis-3-(3-((6-fluoro-1,1-dioxido-2,3-dihydrobenzo[d]isothiazol-5-yl)amino)-1H-pyrazol-5-yl)cyclopentyl propylcarbamate

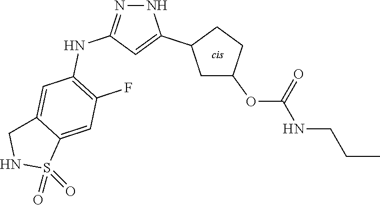 [SMILES: CCCNC(=O)OC1CCC(c2cc(Nc3cc4c(cc3F)S(=O)(=O)NC4)n[nH]2)C1]

The titled compound was synthesized in the procedures similar to Example 34 in a racemic form, which was further separated by Chiral Prep-HPLC to give:

Enantiomer 1 (Example 42a, 99.44% ee); Retention time: 6.023 min. ¹H NMR (500 MHz, DMSO-d₆) δ 11.97 (s, 1H), 8.73 (s, 1H), 8.14 (s, 1H), 7.85-7.83 (m, 2H), 7.04 (t, J=5.5 Hz, 1H), 5.85 (s, 1H), 5.01 (s, 1H), 4.30 (d, J=4.3 Hz, 2H), 3.08 (dd, J=17.1, 8.6 Hz, 1H), 2.92 (dd, J=12.9, 6.6 Hz, 2H), 2.45-2.37 (m, 1H), 2.07-1.97 (m, 1H), 1.97-1.86 (m, 1H), 1.79-1.66 (m, 2H), 1.63-1.53 (m, 1H), 1.44-1.34 (m, 2H), 0.82 (t, J=7.4 Hz, 3H). LC-MS (ESI): m/z [M+H]⁺=438.30.

Enantiomer 2 (Example 42b, 97.65% ee); Retention time: 7.2 min. ¹H NMR (500 MHz, DMSO-d₆) δ 11.97 (s, 1H), 8.73 (s, 1H), 8.14 (s, 1H), 7.85-7.83 (m, 2H), 7.04 (t, J=5.5 Hz, 1H), 5.85 (s, 1H), 5.01 (s, 1H), 4.30 (d, J=4.3 Hz, 2H), 3.08 (dd, J=17.1, 8.6 Hz, 1H), 2.92 (dd, J=12.9, 6.6 Hz, 2H), 2.45-2.37 (m, 1H), 2.07-1.97 (m, 1H), 1.97-1.86 (m, 1H), 1.79-1.66 (m, 2H), 1.63-1.53 (m, 1H), 1.44-1.34 (m, 2H), 0.82 (t, J=7.4 Hz, 3H). LC-MS (ESI): m/z [M+H]⁺=438.30.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=40: 60 (v/v); Flow Rate: 18 mL/min, Wavelength: UV 200 nm and 270 nm, Prep-HPLC Equipment: Prep-HPLC-Gilson, Back pressure: 100 bar; Column temperature: 25° C.

### Example 43: cis-3-(3-((6-fluoro-1,1-dioxido-2,3-dihydrobenzo[d]isothiazol-5-yl)amino)-1H-pyrazol-5-yl)cyclopentyl((S)-sec-butyl)carbamate

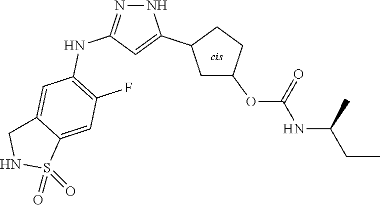 [SMILES: CC[C@H](C)NC(=O)OC1CCC(c2cc(Nc3cc4c(cc3F)S(=O)(=O)NC4)n[nH]2)C1]

The titled compound was synthesized in the procedures similar to Example 34. ¹H NMR (500 MHz, DMSO-d₆) δ 11.90 (s, 1H), 8.66 (s, 1H), 8.08 (s, 1H), 7.65-7.43 (m, 2H), 6.81 (d, J=7.7 Hz, 1H), 5.78 (s, 1H), 4.93 (s, 1H), 4.23 (d, J=5.0 Hz, 2H), 3.37-3.29 (m, 1H), 3.05-2.95 (m, 1H), 2.41-2.34 (m, 1H), 2.03-1.92 (m, 1H), 1.97-1.86 (m, 1H), 1.75-1.59 (m, 2H), 1.63-1.53 (m, 1H), 1.28 (s, 2H), 0.94 (d, J=6.1 Hz, 3H), 0.73 (q, J=6.9 Hz, 3H). LC-MS (ESI): m/z [M+H]⁺=452.30.

### Example 44: cis-3-(3-((6-fluoro-1,1-dioxido-2,3-dihydrobenzo[d]isothiazol-5-yl)amino)-1H-pyrazol-5-yl)cyclopentyl(1-methylcyclopropyl)carbamate

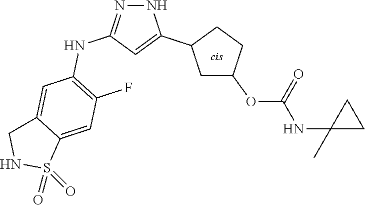 [SMILES: CC1(NC(=O)OC2CCC(c3cc(Nc4cc5c(cc4F)S(=O)(=O)NC5)n[nH]3)C2)CC1]

The titled compound was synthesized in the procedures similar to Example 34 in a racemic form, which was further separated by Chiral Prep-HPLC to give:

Enantiomer 1 (Example 44a, 100% ee); Retention time: 7.49 min. ¹H NMR (500 MHz, DMSO-d₆) δ 11.97 (s, 1H), 8.73 (s, 1H), 8.14 (s, 1H), 7.71-7.43 (m, 2H), 7.34 (s, 1H), 5.84 (s, 1H), 4.99 (s, 1H), 4.30 (d, J=4.8 Hz, 2H), 3.12-3.00 (m, 1H), 2.43-2.38 (m, 1H), 2.10-1.98 (m, 1H), 1.96-1.85 (m, 1H), 1.75-1.65 (m, 2H), 1.58-1.52 (m, 1H), 1.23 (s, 3H), 0.61-0.56 (m, 2H), 0.49-0.45 (m, 2H). LC-MS (ESI): m/z [M+H]⁺=450.2

Enantiomer 2 (Example 44b, 99.6% ee); Retention time: 9.29 min. ¹H NMR (500 MHz, DMSO-d₆) δ 11.97 (s, 1H), 8.73 (s, 1H), 8.14 (s, 1H), 7.71-7.43 (m, 2H), 7.34 (s, 1H), 5.84 (s, 1H), 4.99 (s, 1H), 4.30 (d, J=4.8 Hz, 2H), 3.12-3.00 (m, 1H), 2.43-2.38 (m, 1H), 2.10-1.98 (m, 1H), 1.96-1.85 (m, 1H), 1.75-1.65 (m, 2H), 1.58-1.52 (m, 1H), 1.23 (s, 3H), 0.61-0.56 (m, 2H), 0.49-0.45 (m, 2H). LC-MS (ESI): m/z [M+H]⁺=450.2

Chiral analytical method: Column: CHIRALPAK IE 4.6 mm×250 mm, 5 μm; Mobile phase: A for Hex and B for EtOH (0.2% 2M NH₃ MeOH); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for Hex and B for EtOH (0.2% 2M NH₃ MeOH); Gradient: Mobile Phase A: Mobile Phase B=50: 50 (v/v); Flow Rate: 18 mL/min, Wave Length: UV 270 nm and 310 nm, Prep-HPLC Equipment: Prep-HPLC-Gilson, Back pressure: 100 bar; Column temperature: RT.

### Example 45: cis-3-(3-((6-fluoro-2-methyl-1,1-dioxido-2,3-dihydrobenzo[d]isothiazol-5-yl)amino)-1H-pyrazol-5-yl)cyclopentyl isopropylcarbamate

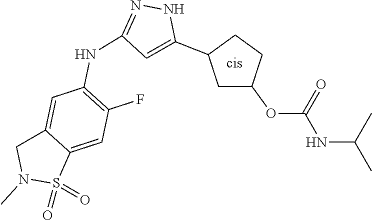 [SMILES: CC(C)NC(=O)OC1CCC(c2cc(Nc3cc4c(cc3F)S(=O)(=O)N(C)C4)n[nH]2)C1]

The titled compound was synthesized in the procedures similar to Example 35. ¹H NMR (500 MHz, DMSO-d₆) δ=11.99 (s, 1H), 8.80 (s, 1H), 8.19 (d, J=7.3, 1H), 7.72 (d, J=10.1, 1H), 6.94 (d, J=7.2, 1H), 5.85 (s, 1H), 5.00 (s, 1H), 4.30 (s, 2H), 3.58 (m, 1H), 3.11-3.00 (m, 1H), 2.76 (s, 3H), 2.48-2.40 (m, 1H), 2.06-1.97 (m, 1H), 1.97-1.83 (m, 1H), 1.70 (m, 2H), 1.59 (m, 1H), 1.03 (m, 6H). LC-MS (ESI): m/z [M+H]⁺=452.3.

### Example 46: cis-3-(3-((6-fluoro-2-(methyl-d3)-1,1-dioxido-2,3-dihydrobenzo[d]isothiazol-5-yl)amino)-1H-pyrazol-5-yl)cyclopentyl isopropylcarbamate

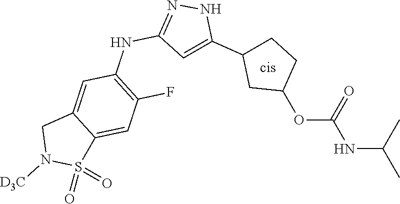 [SMILES: [2H]C([2H])([2H])N1Cc2cc(Nc3cc(C4CCC(OC(=O)NC(C)C)C4)[nH]n3)c(F)cc2S1(=O)=O]

The titled compound was synthesized in the procedures similar to Example 35. ¹H NMR (500 MHz, DMSO-d₆) δ 12.02 (s, 1H), 8.82 (s, 1H), 8.20 (d, J=10 Hz, 1H), 7.72 (d, J=10 Hz, 1H), 6.95 (d, J=5 Hz, 1H), 5.85 (s, 1H), 5.04-4.95 (m, 1H), 4.30 (s, 2H), 3.62-3.53 (m, 1H), 3.12-3.01 (m, 1H), 2.48-2.43 (m, 1H), 2.06-1.98 (m, 1H), 1.96-1.85 (m, 1H), 1.77-1.64 (m, 2H), 1.63-1.54 (m, 1H), 1.07-0.95 (m, 6H). LC-MS (ESI): m/z [M+H]⁺=455.3.

### Example 47: cis-3-(3-((6-fluoro-2-(2-hydroxyethyl)-1,1-dioxido-2,3-dihydrobenzo[d]isothiazol-5-yl)amino)-1H-pyrazol-5-yl)cyclopentyl isopropylcarbamate

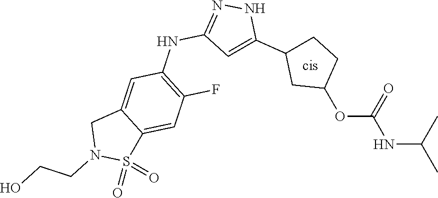 [SMILES: CC(C)NC(=O)OC1CCC(c2cc(Nc3cc4c(cc3F)S(=O)(=O)N(CCO)C4)n[nH]2)C1]

The titled compound was synthesized in the procedures similar to Example 37. ¹H NMR (500 MHz, DMSO-d₆) δ 11.99 (s, 1H), 8.79 (s, 1H), 8.18 (s, 1H), 7.71 (d, J=10.1 Hz, 1H), 6.94 (d, J=7.0 Hz, 1H), 5.85 (s, 1H), 5.00 (s, 1H), 4.88 (t, J=5.5 Hz, 1H), 4.42 (s, 2H), 3.65 (q, J=5.7 Hz, 2H), 3.58 (dd, J=12.9, 6.5 Hz, 1H), 3.18 (t, J=5.9 Hz, 2H), 3.11-2.99 (m, 1H), 2.48-2.41 (m, 1H), 2.07-1.97 (m, 1H), 1.96-1.84 (m, 1H), 1.81-1.64 (m, 2H), 1.62-1.54 (m, 1H), 1.03 (d, J=5.9 Hz, 7H). LC-MS (ESI): m/z [M+H]⁺=482.3.

### Example 48: cis-3-(3-((6-fluoro-2-(2-methoxyethyl)-1,1-dioxido-2,3-dihydrobenzo[d]isothiazol-5-yl)amino-1H-pyrazol-5-yl)cyclopentyl isopropylcarbamate

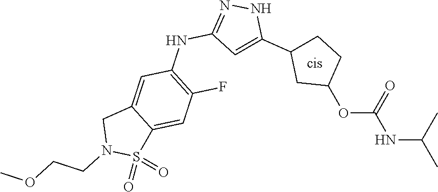 [SMILES: COCCN1Cc2cc(Nc3cc(C4CCC(OC(=O)NC(C)C)C4)[nH]n3)c(F)cc2S1(=O)=O]

The titled compound was synthesized in the procedures similar to Example 35. ¹H NMR (500 MHz, DMSO-d₆) δ 11.92 (s, 1H), 8.73 (s, 1H), 8.15-8.06 (m, 1H), 7.65 (d, J=10 Hz, 1H), 6.86 (d, J=5 Hz, 1H), 5.79 (s, 1H), 4.98-4.88 (m, 1H), 4.34 (s, 2H), 3.51 (d, J=5 Hz, 3H), 3.25 (s, 3H), 3.24-3.21 (m, 2H), 3.04-2.94 (m, 1H), 2.41-2.37 (m, 1H), 1.98-1.92 (m, 1H), 1.88-1.79 (m, 1H), 1.66-1.58 (m, 2H), 1.55-1.50 (m, 1H), 1.0-0.94 (m, 6H). LC-MS (ESI): m/z [M+H]⁺=496.2.

### Example 49: cis-3-(3-((6-fluoro-2-isopropyl-1,1-dioxido-2,3-dihydrobenzo[d]isothiazol-5-yl)amino)-1H-pyrazol-5-yl)cyclopentyl isopropylcarbamate

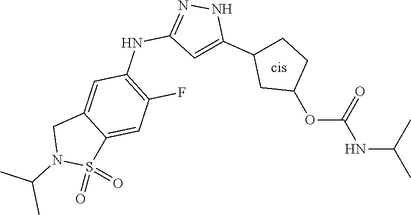 [SMILES: CC(C)NC(=O)OC1CCC(c2cc(Nc3cc4c(cc3F)S(=O)(=O)N(C(C)C)C4)n[nH]2)C1]

The titled compound was synthesized in the procedures similar to Example 35. ¹H NMR (500 MHz, DMSO-d₆) δ 11.93 (s, 1H), 8.64 (s, 1H), 8.06 (s, 1H), 7.52 (d, J=10 Hz, 1H), 6.81 (d, J=5 Hz, 1H), 5.72 (s, 1H), 4.90-4.85 (m, 1H), 4.22 (s, 2H), 3.74-3.66 (m, 1H), 3.48-3.41 (m, 1H), 2.98-2.87 (m, 1H), 2.35-2.30 (m, 1H), 1.95-1.85 (m, 1H), 1.83-1.73 (m, 1H), 1.65-1.53 (m, 2H), 1.50-1.42 (m, 1H), 1.16-1.10 (m, 6H), 0.96-0.84 (m, 6H). LC-MS (ESI): m/z [M+H]⁺=480.2.

### Example 50:3-(3-((6-fluoro-1,1-dioxido-2,3-dihydrobenzo[d]isothiazol-5-yl)amino)-1H-pyrazol-5-yl)cyclobutyl propylcarbamate

### Step 1:3-(3,3-dimethoxycyclobutyl)-3-oxopropanenitrile

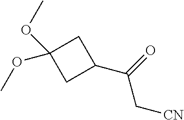

To a solution of Acetonitrile (0.52 g, 12.68 mmol) in THF (20 mL) at −70° C., n-BuLi (4.78 mL, 2.4M, 11.48 mmol) was added dropwise. The mixture was stirred at −70° C. for 30 mins. methyl 3,3-dimethoxycyclobutane-1-carboxylate (1 g, 5.74 mmol) was added and stirred at −50~−70° C. for 60 mins. The mixture was quenched with saturated NH₄Cl (aq.) (10 mL). The resulting mixture was extracted with EtOAc (30 mL×3), the combined organic phases were washed with brine and concentrated under reduced pressure. The residue was purified by silica gel column chromatography, eluting with PE/EtOAc (1:1) to afford the product (0.92 g, 87.6%). LC-MS (ESI): m/z [M+H]⁺=184.

### Step 2:1-(tert-butyl)-3-(3,3-dimethoxycyclobutyl)-1H-pyrazol-5-amine

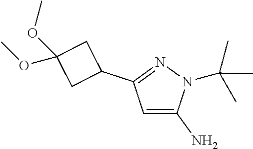 [SMILES: COC1(OC)CC(c2cc(N)n(C(C)(C)C)n2)C1]

To a mixture of 3-(3,3-dimethoxycyclobutyl)-3-oxopropanenitrile (0.9 g, 4.92 mmol) and tert-Butylhydrazine hydrochloride (0.61 g, 4.92 mmol) in EtOH (20 mL), DIEA (0.635 g, 4.92 mmol) was added. The mixture was stirred at 75° C. for 30 mins. The resulting mixture was concentrated under reduced pressure. The residue was purified by silica gel column chromatography, eluting with PE/EtOAc (1: 1~1:5) to afford the product (0.9 g, 72%). LC-MS (ESI): m/z [M+H]⁺=254.2

### Step 3: benzyl(1-(tert-butyl)-3-(3,3-dimethoxycyclobutyl)-1H-pyrazol-5-yl)carbamate

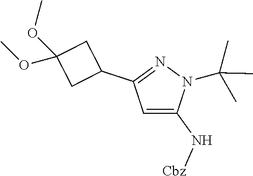 [SMILES: COC1(OC)CC(c2cc(NC(=O)OCc3ccccc3)n(C(C)(C)C)n2)C1]

To a mixture of 1-(tert-butyl)-3-(3,3-dimethoxycyclobutyl)-1H-pyrazol-5-amine (0.9 g, 3.55 mmol) and NaHCO₃ (0.45 g, 5.33 mmol) in Acetonitrile (20 mL), CbzCl (0.67 g, 3.92 mmol) was added. The mixture was stirred at 25° C. for 12 hrs. The resulting mixture was concentrated under reduced pressure. The residue was dissolved in EA (50 mL), washed by brine (30 mL×3) and concentrated to afford the crude product, which was used for next step without purification. (1.2 g, crude). LC-MS (ESI): m/z [M+H]⁺=388.2

### Step 4: benzyl(1-(tert-butyl)-3-(3-oxocyclobutyl)-1H-pyrazol-5-yl)carbamate

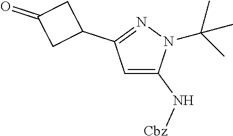 [SMILES: CC(C)(C)n1nc(C2CC(=O)C2)cc1NC(=O)OCc1ccccc1]

To a mixture of benzyl(1-(tert-butyl)-3-(3,3-dimethoxycyclobutyl)-1H-pyrazol-5-yl)carbamate (1.2 g, 4.74 mmol) in Acetonitrile (10 mL) and water (10 mL), PTSA (0.96 g, 5.57 mmol) was added. The mixture was stirred at 60° C. for 12 hrs. The mixture was quenched with saturated NaHCO₃ (aq.) to PH=7 and extracted by EA (30 mL×3). The EA layer was concentrated under reduced pressure. The residue was purified by silica gel column chromatography, eluting with PE/EtOAc (10: 1~5:1) to afford the product (0.7 g, 75%). LC-MS (ESI): m/z [M+H]⁺=342.2

### Step 5: cis-benzyl(1-(tert-butyl)-3-(3-hydroxycyclobutyl)-1H-pyrazol-5-yl)carbamate

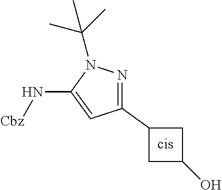 [SMILES: CC(C)(C)n1nc(C2CC(O)C2)cc1NC(=O)OCc1ccccc1]

To a mixture of benzyl(1-(tert-butyl)-3-(3-oxocyclobutyl)-1H-pyrazol-5-yl)carbamate (1.0 g, 2.93 mmol) in EtOH (20 mL), NaBH₄ (0.14 g, 3.68 mmol) was added. The mixture was stirred at 25° C. for 2 hrs. The mixture was quenched with saturated NH₄Cl (aq.) (10 mL) and extracted by DCM (30 mL×3). The DCM layer was concentrated under reduced pressure to afford the crude product, which was used for next step without purification. (0.7 g, crude). LC-MS (ESI): m/z [M+H]⁺=344.2

### Step 6: cis-benzyl(1-(tert-butyl)-3-(3-(((4-nitrophenoxy) carbonyl)oxy)cyclobutyl)-1H-pyrazol-5-yl)carbamate

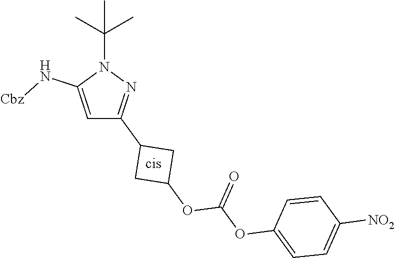 [SMILES: CC(C)(C)n1nc(C2CC(OC(=O)Oc3ccc([N+](=O)[O-])cc3)C2)cc1NC(=O)OCc1ccccc1]

To a mixture of cis-benzyl(1-(tert-butyl)-3-(3-hydroxycyclobutyl)-1H-pyrazol-5-yl)carbamate (0.7 g, 2.04 mmol) and 4-nitrophenyl carbonochloridate (0.615 g, 3.06 mmol) in THF (10 mL), pyridine (0.245 g, 4.08 mmol) was added. The mixture was stirred at 25° C. for 12 hrs. The mixture was concentrated under reduced pressure. The residue was purified by silica gel column chromatography, eluting with PE/EtOAc (5: 1~2:1) to afford the product (0.8 g, 77.6%). LC-MS (ESI): m/z [M+H]⁺=509.2

### Step 7: cis-benzyl(1-(tert-butyl)-3-(3-((propylcarbamoyl)oxy)cyclobutyl)-1H-pyrazol-5-yl)carbamate

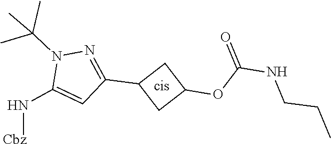 [SMILES: CCCNC(=O)OC1CC(c2cc(NC(=O)OCc3ccccc3)n(C(C)(C)C)n2)C1]

To a mixture of cis-benzyl(1-(tert-butyl)-3-(3-(((4-nitrophenoxy) carbonyl)oxy)cyclobutyl)-1H-pyrazol-5-yl)carbamate (0.7 g, 1.38 mmol) and propylamine (0.13 g, 2.07 mmol) in THF (15 mL), DIEA (0.36 g, 2.76 mmol) was added. The mixture was stirred at 25° C. for 12 hrs. The mixture was concentrated under reduced pressure. The residue was purified by silica gel column chromatography, eluting with DCM/MeOH (10:1) to afford the product (0.5 g, 59%). LC-MS (ESI): m/z [M+H]⁺=429.3

### Step 8: cis-3-(5-amino-1-(tert-butyl)-1H-pyrazol-3-yl)cyclobutyl propylcarbamate

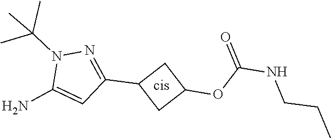 [SMILES: CCCNC(=O)OC1CC(c2cc(N)n(C(C)(C)C)n2)C1]

To a mixture of cis-benzyl(1-(tert-butyl)-3-(3-((propylcarbamoyl)oxy)cyclobutyl)-1H-pyrazol-5-yl)carbamate (0.5 g, 1.17 mmol) in MeOH (10 mL), 10% Pd/C (0.1 g) was added. The mixture was stirred at 25° C. under H₂ balloon for 2 hrs. The mixture was filtered and washed by MeOH (100 mL). The filtrate was concentrated under reduced pressure to afford the crude product (0.4 g, crude), which was used for next step without purification. LC-MS (ESI): m/z [M+H]⁺=295.3

### Step 9: cis-3-(3-((6-fluoro-1,1-dioxido-2,3-dihydrobenzo[d]isothiazol-5-yl)amino)-1H-pyrazol-5-yl)cyclobutyl propylcarbamate

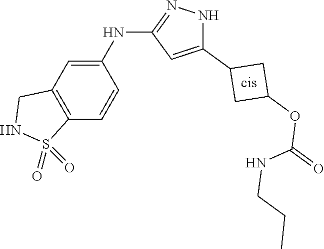 [SMILES: CCCNC(=O)OC1CC(c2cc(Nc3ccc4c(c3)CNS4(=O)=O)n[nH]2)C1]

The titled compound was synthesized in the procedures similar to Example 34, step 4 to step 5. ¹H NMR (500 MHz, DMSO-d₆) δ 12.07 (s, 1H), 8.76 (s, 1H), 8.13 (d, J=7.1 Hz, 1H), 7.67-7.61 (m, 2H), 7.14 (s, 1H), 5.88 (s, 1H), 4.81 (t, J=7.4 Hz, 1H), 4.30 (d, J=4.3 Hz, 2H), 3.12-3.03 (m, 1H), 2.91 (dd, J=13.1, 6.6 Hz, 2H), 2.72-2.63 (m, 2H), 2.11-2.04 (m, 2H), 1.39 (dd, J=14.4, 7.2 Hz, 2H), 0.83 (t, J=7.4 Hz, 3H). LC-MS (ESI): m/z [M+H]⁺=424.2.

### Example 51: cis-3-(3-((4-fluoro-3-methyl-1,1-dioxido-2,3-dihydrobenzo[d]isothiazol-5-yl)amino)-1H-pyrazol-5-yl)cyclopentyl isopropylcarbamate

### Step 1:5-bromo-3-chloro-4-fluorobenzo[d]isothiazole 1,1-dioxide

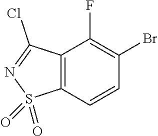 [SMILES: O=S1(=O)N=C(Cl)c2c1ccc(Br)c2F]

A mixture of 5-bromo-4-fluorobenzo[d]isothiazol-3 (2H)-one 1,1-dioxide (1 g, 3.58 mmol) in POCl₃ (15 mL) was stirred at 110° C. for 2 days. LCMS showed the reaction was complete. The mixture was concentrated under vacuum. The residue was treated with EA (30 mL) to afford product (870 mg, 81.3%), which was used in the next step directly. LC-MS (ESI): m/z [M+H]⁺=298.2.

### Step 2:5-bromo-4-fluoro-3-methylbenzo[d]isothiazole 1,1-dioxide

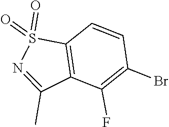

To a mixture of 5-bromo-3-chloro-4-fluorobenzo[d]isothiazole 1,1-dioxide (300 mg, 1.01 mmol) in THF (10 mL) was added Methylmagnesium Bromide (1.01 mL, IN) at 0° C. under a nitrogen atmosphere. The mixture was stirred at 0° C. for 3 h. LCMS showed the reaction was complete. The mixture was quenched by water (20 mL), extracted with EA (3×30 mL). The combined organic layers were dried over Na₂SO₄, filtered and concentrated under vacuum to afford product (250 mg, crude), which was used in the next step directly. LC-MS (ESI): m/z [M+H]⁺=278.2.

### Step 3:5-bromo-4-fluoro-3-methyl-2,3-dihydrobenzo[d]isothiazole 1,1-dioxide

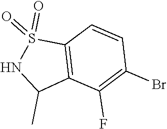

To a solution of 5-bromo-4-fluoro-3-methylbenzo[d]isothiazole 1,1-dioxide (250 mg, 0.90 mmol) in MeOH (10 mL) was added NaBH₄ (41 mg, 1.08 mmol) at 0° C. Th mixture was stirred ar rt for 2 h. LCMS showed the reaction was complete. The mixture was quenched by ice water (50 mL), extracted with EA (3×30 mL). The combined organic layers were dried over Na₂SO₄, filtered and concentrated under vacuum. The residue was purified by silica gel column chromatography, eluting with PE/EA (0-40%) to afford the product (200 mg, 79.4%). LC-MS (ESI): m/z [M+H]⁺=280.1.

### Step 4: cis-3-(3-((4-fluoro-3-methyl-1,1-dioxido-2,3-dihydrobenzo[d]isothiazol-5-yl)amino)-1H-pyrazol-5-yl)cyclopentyl isopropylcarbamate

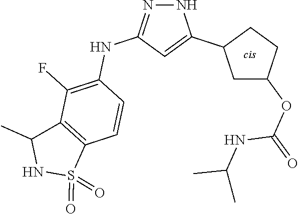 [SMILES: CC(C)NC(=O)OC1CCC(c2cc(Nc3ccc4c(c3F)C(C)NS4(=O)=O)n[nH]2)C1]

A mixture of 5-bromo-4-fluoro-3-methyl-2,3-dihydrobenzo[d]isothiazole 1,1-dioxide (200 mg, 0.72 mmol), cis-3-(3-amino-1H-pyrazol-5-yl)cyclopentyl isopropylcarbamate (144.5 mg, 0.57 mmol), Brettphos Pd G3 (66.1 mg, 0.073 mmol) and K₂CO₃ (298 mg, 2.16 mmol) in t-BuOH (10 mL) was stirred at 110° C. for 16 h under a nitrogen atmosphere. LCMS showed the reaction was complete. The mixture was filtered and the filtrate was concentrated under vacuum. The residue was purified by prep-HPLC (Waters SunFire C18: 19×150 mm, 5 μm, eluting with 30%-50% of acetonitrile (containing 0.1% FA) in water (containing 0.1% FA)) to afford the product in racemic form, which was further separated by Chiral Prep-HPLC to give:

Enantiomer 1 (Example 51a, 100% ee); Retention time: 3.83 min. ¹H NMR (500 MHz, DMSO-d₆) δ 11.99 (s, 1H), 8.69 (s, 1H), 8.24 (t, J=7.5 Hz, 1H), 7.83 (d, J=4.4 Hz, 1H), 7.45 (d, J=8.6 Hz, 1H), 6.94 (d, J=7.3 Hz, 1H), 5.83 (s, 1H), 5.00 (s, 1H), 4.84-4.71 (m, 1H), 3.65-3.58 (m, 1H), 3.13-2.97 (m, 1H), 2.55-2.46 (m, 1H), 2.03-1.98 (m, 1H), 1.96-1.85 (m, 1H), 1.71-1.65 (m, 2H), 1.58-1.48 (m, 1H), 1.49 (d, J=6.6 Hz, 3H), 1.03 (d, J=5.8 Hz, 6H). LC-MS (ESI): m/z [M+H]⁺=452.2.

Enantiomer 2 (Example 51b, 100% ee); Retention time: 4.86 min. ¹H NMR (500 MHz, DMSO-d₆) δ 11.99 (s, 1H), 8.69 (s, 1H), 8.24 (t, J=7.5 Hz, 1H), 7.83 (d, J=4.4 Hz, 1H), 7.45 (d, J=8.6 Hz, 1H), 6.94 (d, J=7.3 Hz, 1H), 5.83 (s, 1H), 5.00 (s, 1H), 4.84-4.71 (m, 1H), 3.65-3.58 (m, 1H), 3.13-2.97 (m, 1H), 2.55-2.46 (m, 1H), 2.03-1.98 (m, 1H), 1.96-1.85 (m, 1H), 1.71-1.65 (m, 2H), 1.58-1.48 (m, 1H), 1.49 (d, J=6.6 Hz, 3H), 1.03 (d, J=5.8 Hz, 6H). LC-MS (ESI): m/z [M+H]⁺=452.2.

Enantiomer 3 (Example 51c, 100% ee); Retention time: 5.56 min. ¹H NMR (500 MHz, DMSO-d₆) δ 11.99 (s, 1H), 8.69 (s, 1H), 8.24 (t, J=7.5 Hz, 1H), 7.83 (d, J=4.4 Hz, 1H), 7.45 (d, J=8.6 Hz, 1H), 6.94 (d, J=7.3 Hz, 1H), 5.83 (s, 1H), 5.00 (s, 1H), 4.84-4.71 (m, 1H), 3.65-3.58 (m, 1H), 3.13-2.97 (m, 1H), 2.55-2.46 (m, 1H), 2.03-1.98 (m, 1H), 1.96-1.85 (m, 1H), 1.71-1.65 (m, 2H), 1.58-1.48 (m, 1H), 1.49 (d, J=6.6 Hz, 3H), 1.03 (d, J=5.8 Hz, 6H). LC-MS (ESI): m/z [M+H]⁺=452.2.

Enantiomer 4 (Example 51d, 100% ee); Retention time: 8.36 min. ¹H NMR (500 MHz, DMSO-d₆) δ 11.99 (s, 1H), 8.69 (s, 1H), 8.24 (t, J=7.5 Hz, 1H), 7.83 (d, J=4.4 Hz, 1H), 7.45 (d, J=8.6 Hz, 1H), 6.94 (d, J=7.3 Hz, 1H), 5.83 (s, 1H), 5.00 (s, 1H), 4.84-4.71 (m, 1H), 3.65-3.58 (m, 1H), 3.13-2.97 (m, 1H), 2.55-2.46 (m, 1H), 2.03-1.98 (m, 1H), 1.96-1.85 (m, 1H), 1.71-1.65 (m, 2H), 1.58-1.48 (m, 1H), 1.49 (d, J=6.6 Hz, 3H), 1.03 (d, J=5.8 Hz, 6H). LC-MS (ESI): m/z [M+H]⁺=452.2.

Chiral analytical method: Column: CHIRALPAK IE 4.6 mm×250 mm, 5 μm; Mobile phase: A for MtBE (0.1% 2M NH₃ MeOH) and B for EtOH; Gradient: Mobile Phase A: Mobile Phase B=30: 70 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for EtOH (0.2% 2M NH₃ MeOH); Gradient: Mobile Phase A: Mobile Phase B=30: 70 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 52: cis-3-(3-((4-fluoro-3-methyl-1,1-dioxido-2,3-dihydrobenzo[d]isothiazol-5-yl)amino)-1H-pyrazol-5-yl)cyclopentyl(1-methylcyclopropyl)carbamate

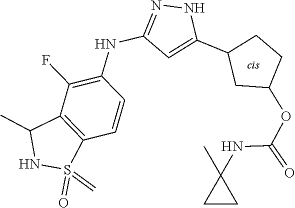 [SMILES: C=S1(=O)NC(C)c2c1ccc(Nc1cc(C3CCC(OC(=O)NC4(C)CC4)C3)[nH]n1)c2F]

The titled compound was synthesized in the procedures similar to Example 51 in a racemic form, which was further separated by Chiral Prep-HPLC to give:

Enantiomer 1 (Example 52a, 100% ee); Retention time: 4.57 min. ¹H NMR (500 MHz, DMSO-d₆) δ 11.98 (s, 1H), 8.68 (s, 1H), 8.23 (s, 1H), 7.82 (d, J=4.7 Hz, 1H), 7.45 (d, J=8.6 Hz, 1H), 7.34 (s, 1H), 5.82 (s, 1H), 4.98 (s, 1H), 4.86-4.69 (m, 1H), 3.132-2.95 (m, 1H), 2.43-2.38 (m, 1H), 2.10-1.98 (m, 1H), 1.96-1.85 (m, 1H), 1.75-1.65 (m, 2H), 1.58-1.52 (m, 1H), 1.49 (d, J=6.6 Hz, 3H), 1.23 (s, 3H), 0.64-0.52 (m, 2H), 0.48-0.40 (m, 2H). LC-MS (ESI): m/z [M+H]⁺=464.2

Enantiomer 2 (Example 52b, 100% ee); Retention time: 5.81 min. ¹H NMR (500 MHz, DMSO-d₆) δ 11.98 (s, 1H), 8.68 (s, 1H), 8.23 (s, 1H), 7.82 (d, J=4.7 Hz, 1H), 7.45 (d, J=8.6 Hz, 1H), 7.34 (s, 1H), 5.82 (s, 1H), 4.98 (s, 1H), 4.86-4.69 (m, 1H), 3.132-2.95 (m, 1H), 2.43-2.38 (m, 1H), 2.10-1.98 (m, 1H), 1.96-1.85 (m, 1H), 1.75-1.65 (m, 2H), 1.58-1.52 (m, 1H), 1.49 (d, J=6.6 Hz, 3H), 1.23 (s, 3H), 0.64-0.52 (m, 2H), 0.48-0.40 (m, 2H). LC-MS (ESI): m/z [M+H]⁺=464.2

Enantiomer 3 (Example 52c, 96.1% ee); Retention time: 6.65 min. ¹H NMR (500 MHz, DMSO-d₆) δ 11.98 (s, 1H), 8.68 (s, 1H), 8.23 (s, 1H), 7.82 (d, J=4.7 Hz, 1H), 7.45 (d, J=8.6 Hz, 1H), 7.34 (s, 1H), 5.82 (s, 1H), 4.98 (s, 1H), 4.86-4.69 (m, 1H), 3.132-2.95 (m, 1H), 2.43-2.38 (m, 1H), 2.10-1.98 (m, 1H), 1.96-1.85 (m, 1H), 1.75-1.65 (m, 2H), 1.58-1.52 (m, 1H), 1.49 (d, J=6.6 Hz, 3H), 1.23 (s, 3H), 0.64-0.52 (m, 2H), 0.48-0.40 (m, 2H). LC-MS (ESI): m/z [M+H]⁺=464.2

Enantiomer 4 (Example 52d, 99.8% ee); Retention time: 11.98 min. ¹H NMR (500 MHz, DMSO-d₆) δ 11.98 (s, 1H), 8.68 (s, 1H), 8.23 (s, 1H), 7.82 (d, J=4.7 Hz, 1H), 7.45 (d, J=8.6 Hz, 1H), 7.34 (s, 1H), 5.82 (s, 1H), 4.98 (s, 1H), 4.86-4.69 (m, 1H), 3.132-2.95 (m, 1H), 2.43-2.38 (m, 1H), 2.10-1.98 (m, 1H), 1.96-1.85 (m, 1H), 1.75-1.65 (m, 2H), 1.58-1.52 (m, 1H), 1.49 (d, J=6.6 Hz, 3H), 1.23 (s, 3H), 0.64-0.52 (m, 2H), 0.48-0.40 (m, 2H). LC-MS (ESI): m/z [M+H]⁺=464.2

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=60: 40 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 53: cis-3-(3-((4-fluoro-3-methyl-1,1-dioxido-2,3-dihydrobenzo[d]isothiazol-5-yl)amino)-1H-pyrazol-5-yl)cyclopentyl propylcarbamate

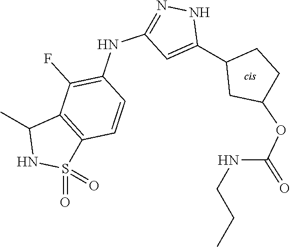 [SMILES: CCCNC(=O)OC1CCC(c2cc(Nc3ccc4c(c3F)C(C)NS4(=O)=O)n[nH]2)C1]

The titled compound was synthesized in the procedures similar to Example 51 in a racemic form, which was further separated by Chiral Prep-HPLC to give:

Enantiomer 1 (Example 53a, 100% ee); Retention time: 4.02 min. ¹H NMR (500 MHz, DMSO-d₆) δ 11.99 (s, 1H), 8.69 (s, 1H), 8.24 (t, J=8.0 Hz, 1H), 7.83 (d, J=4.5 Hz, 1H), 7.45 (d, J=8.6 Hz, 1H), 7.04 (t, J=5.5 Hz, 1H), 5.83 (d, J=1.9 Hz, 1H), 5.01 (s, 1H), 4.84-4.77 (m, 1H), 3.13-3.02 (m, 1H), 2.95-2.89 (m, 2H), 2.47-2.40 (m, 1H), 2.10-1.98 (m, 1H), 1.96-1.86 (m, 1H), 1.75-1.65 (m, 2H), 1.63-1.56 (m, 1H), 1.49 (d, J=6.7 Hz, 3H), 1.44-1.34 (m, 3H), 0.82 (t, J=7.4 Hz, 3H). LC-MS (ESI): m/z [M+H]⁺=452.2

Enantiomer 2 (Example 53b, 100% ee); Retention time: 7.19 min. ¹H NMR (500 MHz, DMSO-d₆) δ 11.99 (s, 1H), 8.69 (s, 1H), 8.24 (t, J=8.0 Hz, 1H), 7.83 (d, J=4.5 Hz, 1H), 7.45 (d, J=8.6 Hz, 1H), 7.04 (t, J=5.5 Hz, 1H), 5.83 (d, J=1.9 Hz, 1H), 5.01 (s, 1H), 4.84-4.77 (m, 1H), 3.13-3.02 (m, 1H), 2.95-2.89 (m, 2H), 2.47-2.40 (m, 1H), 2.10-1.98 (m, 1H), 1.96-1.86 (m, 1H), 1.75-1.65 (m, 2H), 1.63-1.56 (m, 1H), 1.49 (d, J=6.7 Hz, 3H), 1.44-1.34 (m, 3H), 0.82 (t, J=7.4 Hz, 3H). LC-MS (ESI): m/z [M+H]⁺=452.2.

Enantiomer 3 (Example 53c, 98.6% ee); Retention time: 8.43 min. ¹H NMR (500 MHz, DMSO-d₆) δ 11.99 (s, 1H), 8.69 (s, 1H), 8.24 (t, J=8.0 Hz, 1H), 7.83 (d, J=4.5 Hz, 1H), 7.45 (d, J=8.6 Hz, 1H), 7.04 (t, J=5.5 Hz, 1H), 5.83 (d, J=1.9 Hz, 1H), 5.01 (s, 1H), 4.84-4.77 (m, 1H), 3.13-3.02 (m, 1H), 2.95-2.89 (m, 2H), 2.47-2.40 (m, 1H), 2.10-1.98 (m, 1H), 1.96-1.86 (m, 1H), 1.75-1.65 (m, 2H), 1.63-1.56 (m, 1H), 1.49 (d, J=6.7 Hz, 3H), 1.44-1.34 (m, 3H), 0.82 (t, J=7.4 Hz, 3H). LC-MS (ESI): m/z [M+H]⁺=452.2.

Enantiomer 4 (Example 53d, 100% ee); Retention time: 9.11 min. ¹H NMR (500 MHz, DMSO-d₆) δ 11.99 (s, 1H), 8.69 (s, 1H), 8.24 (t, J=8.0 Hz, 1H), 7.83 (d, J=4.5 Hz, 1H), 7.45 (d, J=8.6 Hz, 1H), 7.04 (t, J=5.5 Hz, 1H), 5.83 (d, J=1.9 Hz, 1H), 5.01 (s, 1H), 4.84-4.77 (m, 1H), 3.13-3.02 (m, 1H), 2.95-2.89 (m, 2H), 2.47-2.40 (m, 1H), 2.10-1.98 (m, 1H), 1.96-1.86 (m, 1H), 1.75-1.65 (m, 2H), 1.63-1.56 (m, 1H), 1.49 (d, J=6.7 Hz, 3H), 1.44-1.34 (m, 3H), 0.82 (t, J=7.4 Hz, 3H). LC-MS (ESI): m/z [M+H]⁺=452.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=40: 60 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 54: cis-3-(3-((4-fluoro-3-methyl-1,1-dioxido-2,3-dihydrobenzo[d]isothiazol-5-yl)amino)-1H-pyrazol-5-yl)cyclopentyl((S)-sec-butyl)carbamate

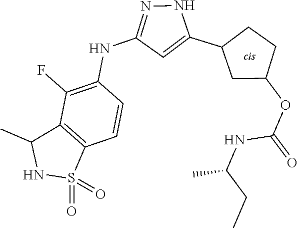 [SMILES: CC[C@H](C)NC(=O)OC1CCC(c2cc(Nc3ccc4c(c3F)C(C)NS4(=O)=O)n[nH]2)C1]

The titled compound was synthesized in the procedures similar to Example 51 in a racemic form, which was further separated by Chiral Prep-HPLC to give:

Enantiomer 1 (Example 54a, 100% ee); Retention time: 3.82 min. ¹H NMR (500 MHz, DMSO-d₆) δ 11.99 (s, 1H), 8.68 (s, 1H), 8.23 (s, 1H), 7.83 (d, J=4.6 Hz, 1H), 7.45 (d, J=8.6 Hz, 1H), 6.88 (d, J=8.2 Hz, 1H), 5.83 (s, 1H), 5.04-4.96 (m, 1H), 4.88-4.70 (m, 1H), 3.43-3.35 (m, 1H), 3.13-3.00 (m, 1H), 2.47-2.40 (m, 1H), 2.06-1.98 (m, 1H), 1.96-1.86 (m, 1H), 1.75-1.65 (m, 2H), 1.63-1.56 (m, 1H), 1.49 (d, J=6.7 Hz, 3H), 1.43-1.30 (m, 2H), 1.01 (d, J=6.5 Hz, 3H), 0.80 (t, J=7.4 Hz, 3H). LC-MS (ESI): m/z [M+H]⁺=466.2.

Enantiomer 2 (Example 54b, 99.7% ee); Retention time: 4.86 min. ¹H NMR (500 MHz, DMSO-d₆) δ 11.99 (s, 1H), 8.68 (s, 1H), 8.23 (s, 1H), 7.83 (d, J=4.6 Hz, 1H), 7.45 (d, J=8.6 Hz, 1H), 6.88 (d, J=8.2 Hz, 1H), 5.83 (s, 1H), 5.04-4.96 (m, 1H), 4.88-4.70 (m, 1H), 3.43-3.35 (m, 1H), 3.13-3.00 (m, 1H), 2.47-2.40 (m, 1H), 2.06-1.98 (m, 1H), 1.96-1.86 (m, 1H), 1.75-1.65 (m, 2H), 1.63-1.56 (m, 1H), 1.49 (d, J=6.7 Hz, 3H), 1.43-1.30 (m, 2H), 1.01 (d, J=6.5 Hz, 3H), 0.80 (t, J=7.4 Hz, 3H). LC-MS (ESI): m/z [M+H]⁺=466.2.

Enantiomer 3 (Example 54c, 96.5% ee); Retention time: 5.21 min. ¹H NMR (500 MHz, DMSO-d₆) δ 11.99 (s, 1H), 8.68 (s, 1H), 8.23 (s, 1H), 7.83 (d, J=4.6 Hz, 1H), 7.45 (d, J=8.6 Hz, 1H), 6.88 (d, J=8.2 Hz, 1H), 5.83 (s, 1H), 5.04-4.96 (m, 1H), 4.88-4.70 (m, 1H), 3.43-3.35 (m, 1H), 3.13-3.00 (m, 1H), 2.47-2.40 (m, 1H), 2.06-1.98 (m, 1H), 1.96-1.86 (m, 1H), 1.75-1.65 (m, 2H), 1.63-1.56 (m, 1H), 1.49 (d, J=6.7 Hz, 3H), 1.43-1.30 (m, 2H), 1.01 (d, J=6.5 Hz, 3H), 0.80 (t, J=7.4 Hz, 3H). LC-MS (ESI): m/z [M+H]⁺=466.2.

Enantiomer 4 (Example 54d, 100% ee); Retention time: 9.68 min. ¹H NMR (500 MHz, DMSO-d₆) δ 11.99 (s, 1H), 8.68 (s, 1H), 8.23 (s, 1H), 7.83 (d, J=4.6 Hz, 1H), 7.45 (d, J=8.6 Hz, 1H), 6.88 (d, J=8.2 Hz, 1H), 5.83 (s, 1H), 5.04-4.96 (m, 1H), 4.88-4.70 (m, 1H), 3.43-3.35 (m, 1H), 3.13-3.00 (m, 1H), 2.47-2.40 (m, 1H), 2.06-1.98 (m, 1H), 1.96-1.86 (m, 1H), 1.75-1.65 (m, 2H), 1.63-1.56 (m, 1H), 1.49 (d, J=6.7 Hz, 3H), 1.43-1.30 (m, 2H), 1.01 (d, J=6.5 Hz, 3H), 0.80 (t, J=7.4 Hz, 3H). LC-MS (ESI): m/z [M+H]⁺=466.2.

Chiral analytical method: Column: CHIRALPAK IE 4.6 mm×250 mm, 5 μm; Mobile phase: A for MtBE (0.1% 2M NH₃ MeOH) and B for EtOH; Gradient: Mobile Phase A: Mobile Phase B=30: 70 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for EtOH (0.2% 2M NH₃ MeOH); Gradient: Mobile Phase A: Mobile Phase B=30: 70 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 55: cis-3-(3-((4-fluoro-3,3-dimethyl-1,1-dioxido-2,3-dihydrobenzo[d]isothiazol-5-yl)amino)-1H-pyrazol-5-yl)cyclopentyl isopropylcarbamate

### Step 1:5-bromo-4-fluoro-3,3-dimethyl-2,3-dihydrobenzo[d]isothiazole 1,1-dioxide

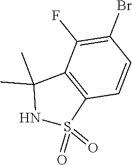

The titled compound was synthesized in the procedures similar to Example 8, step 1 to step 2.

LC-MS (ESI): m/z [M+H]⁺=294.0.

### Step 2: cis-3-(3-((4-fluoro-3,3-dimethyl-1,1-dioxido-2,3-dihydrobenzo[d]isothiazol-5-yl)amino)-1H-pyrazol-5-yl)cyclopentyl isopropylcarbamate

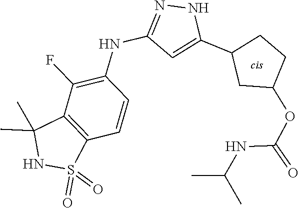 [SMILES: CC(C)NC(=O)OC1CCC(c2cc(Nc3ccc4c(c3F)C(C)(C)NS4(=O)=O)n[nH]2)C1]

The titled compound was synthesized in the procedures similar to Example 34, step 5. ¹H NMR (500 MHz, DMSO-d₆) δ=11.99 (s, 1H), 8.66 (s, 1H), 8.19 (s, 1H), 7.84 (s, 1H), 7.42 (d, J=8.6, 1H), 6.93 (s, 1H), 5.83 (s, 1H), 5.00 (s, 1H), 3.57 (m, 1H), 3.13-3.01 (m, 1H), 2.47 (m, 1H), 2.03 (m, 1H), 1.95-1.84 (m, 1H), 1.71 (m, 2H), 1.60 (m, 7H), 1.07-0.96 (m, 6H). LC-MS (ESI): m/z [M+H]⁺=466.3.

### Example 56: cis-3-(3-((4-fluoro-3,3-dimethyl-1,1-dioxido-2,3-dihydrobenzo[d]isothiazol-5-yl)amino)-1H-pyrazol-5-yl)cyclopentyl(1-methylcyclopropyl)carbamate

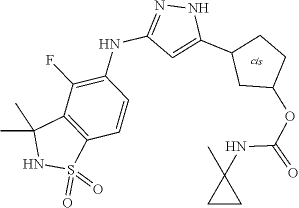 [SMILES: CC1(NC(=O)OC2CCC(c3cc(Nc4ccc5c(c4F)C(C)(C)NS5(=O)=O)n[nH]3)C2)CC1]

The titled compound was synthesized in the procedures similar to Example 55. ¹H NMR (500 MHz, DMSO-d₆) δ 11.99 (s, 1H), 8.66 (s, 1H), 8.20 (s, 1H), 7.84 (s, 1H), 7.42 (d, J=8.6 Hz, 1H), 7.34 (s, 1H), 5.82 (s, 1H), 3.13-2.99 (m, 1H), 2.50-2.45 (m, 1H), 2.03-1.98 (m, 1H), 1.96-1.85 (m, 1H), 1.71-1.65 (m, 2H), 1.58-1.48 (m, 1H), 1.60 (s, 6H), 1.23 (s, 6H), 0.59 (s, 2H), 0.47 (t, J=5.3 Hz, 2H).

LC-MS (ESI): m/z [M+H]⁺=478.2.

### Example 57: cis-3-(3-((4-fluoro-3,3-dimethyl-1,1-dioxido-2,3-dihydrobenzo[d]isothiazol-5-yl)amino)-1H-pyrazol-5-yl)cyclopentyl((S)-sec-butyl)carbamate

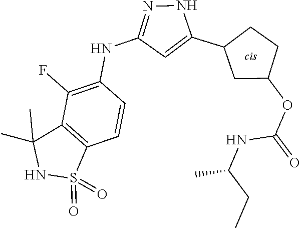 [SMILES: CC[C@H](C)NC(=O)OC1CCC(c2cc(Nc3ccc4c(c3F)C(C)(C)NS4(=O)=O)n[nH]2)C1]

The titled compound was synthesized in the procedures similar to Example 55. ¹H NMR (500 MHz, DMSO-d₆) δ 11.99 (s, 1H), 8.69 (s, 1H), 8.20 (d, J=32.0 Hz, 1H), 7.83 (d, J=4.5 Hz, 1H), 7.45 (d, J=8.6 Hz, 1H), 6.88 (d, J=7.9 Hz, 1H), 5.83 (s, 1H), 4.84-4.71 (m, 1H), 3.37 (dd, J=13.8, 7.1 Hz, 1H), 3.12-3.01 (m, 1H), 2.50-2.45 (m, 1H), 2.07-1.97 (m, 1H), 1.96-1.85 (m, 1H), 1.71-1.65 (m, 2H), 1.58-1.48 (m, 1H), 1.49 (s, 6H), 1.36 (d, J=5.0 Hz, 2H), 1.06-0.95 (m, 3H), 0.80 (q, J=7.2 Hz, 3H). LC-MS (ESI): m/z [M+H]⁺=480.2.

### Example 58: cis-3-(3-((4-fluoro-3,3-dimethyl-1,1-dioxido-2,3-dihydrobenzo[d]isothiazol-5-yl)amino)-1H-pyrazol-5-yl)cyclopentyl propylcarbamate

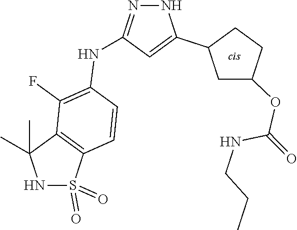 [SMILES: CCCNC(=O)OC1CCC(c2cc(Nc3ccc4c(c3F)C(C)(C)NS4(=O)=O)n[nH]2)C1]

The titled compound was synthesized in the procedures similar to Example 55. ¹H NMR (500 MHz, DMSO-d₆) δ 11.99 (s, 1H), 8.69 (s, 1H), 8.23 (s, 1H), 7.83 (d, J=4.5 Hz, 1H), 7.45 (d, J=8.6 Hz, 1H), 7.04 (t, J=5.2 Hz, 1H), 5.83 (s, 1H), 4.83-4.77 (m, 1H), 3.10-3.02 (m, 1H), 2.94-2.89 (m, 2H), 2.50-2.45 (m, 1H), 2.06-1.99 (m, 1H), 1.96-1.86 (m, 1H), 1.78-1.66 (m, 2H), 1.65-1.57 (m, 1H), 1.43 (d, J=6.6 Hz, 6H), 1.43-1.35 (m, 2H), 0.82 (t, J=7.4 Hz, 3H). LC-MS (ESI): m/z [M+H]⁺=466.2.

### Example 59: cis-3-(3-((4-fluoro-1,1-dioxido-3-oxo-2,3-dihydrobenzo[d]isothiazol-5-yl)amino)-1H-pyrazol-5-yl)cyclopentyl isopropylcarbamate

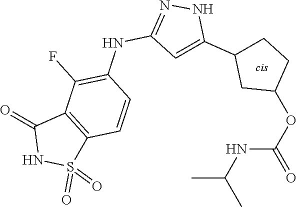 [SMILES: CC(C)NC(=O)OC1CCC(c2cc(Nc3ccc4c(c3F)C(=O)NS4(=O)=O)n[nH]2)C1]

The titled compound was synthesized in the procedures similar to Example 8. ¹H NMR (500 MHz, DMSO-d₆) δ 12.05 (s, 1H), 8.92 (s, 1H), 8.47 (s, 1H), 7.62 (d, J=8.6 Hz, 1H), 6.95 (d, J=7.8 Hz, 1H), 5.86 (s, 1H), 5.00 (s, 1H), 3.62-3.55 (m, 1H), 3.13-3.04 (m, 1H), 2.46-2.44 (m, 1H), 2.08-1.98 (m, 1H), 1.98-1.88 (m, 1H), 1.78-1.66 (m, 2H), 1.65-1.55 (m, 1H), 1.03 (d, J=6.2 Hz, 6H). LC-MS (ESI): m/z [M+H]⁺=452.3.

### Example 60: cis-3-(3-((2-ethyl-4-fluoro-1,1-dioxido-2,3-dihydrobenzo[d]isothiazol-5-yl)amino)-1H-pyrazol-5-yl)cyclopentyl isopropylcarbamate

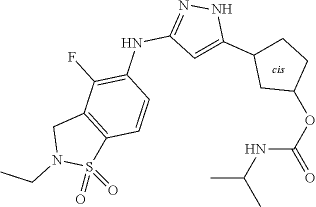 [SMILES: CCN1Cc2c(ccc(Nc3cc(C4CCC(OC(=O)NC(C)C)C4)[nH]n3)c2F)S1(=O)=O]

The titled compound was synthesized in the procedures similar to Example 35. ¹H NMR (500 MHz, DMSO-d₆) δ 12.00 (s, 1H), 8.77 (s, 1H), 8.29 (s, 1H), 7.52 (d, J=8.6 Hz, 1H), 6.94 (d, J=7.2 Hz, 1H), 5.84 (s, 1H), 5.00 (s, 1H), 4.46 (s, 2H), 3.69-3.50 (m, 1H), 3.20 (q, J=7.2 Hz, 2H), 3.12-3.00 (m, 1H), 2.50-2.46 (m, 1H), 2.06-1.99 (m, 1H), 1.96-1.86 (m, 1H), 1.78-1.66 (m, 2H), 1.65-1.57 (m, 1H), 1.26 (t, J=7.2 Hz, 3H), 1.03 (d, J=6.2 Hz, 6H). LC-MS (ESI): m/z [M+H]⁺=466.2.

### Example 61: cis-3-(3-((4-fluoro-2-(1-methylpiperidin-3-yl)-1,1-dioxido-2,3-dihydrobenzo[d]isothiazol-5-yl)amino)-1H-pyrazol-5-yl)cyclopentyl isopropylcarbamate

### Step 1:1-methylpiperidin-3-yl methanesulfonate

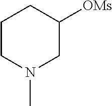

To a solution of 1-methylpiperidin-3-ol (0.35 g, 3.04 mmol) and TEA (0.47 g, 4.56 mmol) in DCM (10 mL), MsCl (0.38 g, 3.34 mmol) was added at 0° C. Then the mixture was stirred at 25° C. for 1 hr. The mixture was diluted by DCM (20 mL) and washed with brine (30 mL×3). The DCM layer was concentrated under reduced pressure to afford the product, which was used for next step without purification. (0.45 g, 77.5%). LC-MS (ESI): m/z [M+H]⁺=194.

### Step 2:5-bromo-4-fluoro-2-(1-methylpiperidin-3-yl)-2,3-dihydrobenzo[d]isothiazole 1,1-dioxide

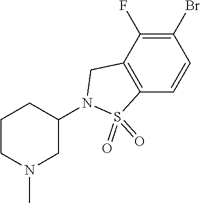 [SMILES: CN1CCCC(N2Cc3c(ccc(Br)c3F)S2(=O)=O)C1]

A mixture of 1-methylpiperidin-3-yl methanesulfonate (0.3 g, 1.55 mmol), Cs₂CO₃ (0.375 g, 1.14 mmol) and 5-bromo-4-fluoro-2,3-dihydrobenzo[d]isothiazole 1,1-dioxide (0.15 g, 0.56 mmol) in DMF (5 mL) was stirred at 80° C. for 3 hr. The mixture was quenched with water (20 mL). The mixture was extracted by DCM (20 mL×3). The DCM layer was concentrated under reduced pressure. The residue was purified by silica gel column chromatography, eluting with PE/EtOAc (10: 1~5:1) to afford the product (0.15 g, 75%). LC-MS (ESI): m/z [M+Na]⁺=385.3.

### Step 3: cis-3-(3-((4-fluoro-2-(1-methylpiperidin-3-yl)-1,1-dioxido-2,3-dihydrobenzo[d]isothiazol-5-yl)amino)-1H-pyrazol-5-yl)cyclopentyl isopropylcarbamate

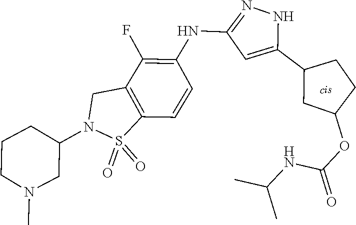 [SMILES: CC(C)NC(=O)OC1CCC(c2cc(Nc3ccc4c(c3F)CN(C3CCCN(C)C3)S4(=O)=O)n[nH]2)C1]

The titled compound was synthesized in the procedures similar to Example 34, step 3 to step 4. ¹H NMR (500 MHz, DMSO-d₆)) δ 12.00 (s, 1H), 8.77 (s, 1H), 8.29 (s, 1H), 7.54 (d, J=8.6 Hz, 1H), 6.94 (d, J=7.0 Hz, 1H), 5.83 (s, 1H), 5.00 (s, 1H), 4.57 (dd, J=61.0, 14.3 Hz, 2H), 3.63-3.52 (m, 1H), 3.27 (d, J=3.8 Hz, 1H), 3.12-2.94 (m, 3H), 2.48-2.43 (m, 1H), 2.35 (s, 3H), 2.22-2.11 (m, 1H), 2.05-1.98 (m, 1H), 1.95-1.85 (m, 2H), 1.76-1.56 (m, 7H), 1.03 (d, J=6.2 Hz, 6H).). LC-MS (ESI): m/z [M+H]⁺=535.3.

### Example 62: cis-3-(3-((4-fluoro-2-(1-methylpiperidin-4-yl)-1,1-dioxido-2,3-dihydrobenzo[d]isothiazol-5-yl)amino)-1H-pyrazol-5-yl)cyclopentyl isopropylcarbamate

### Step 1:5-bromo-4-fluoro-2-(1-methylpiperidin-4-yl)-2,3-dihydrobenzo[d]isothiazole 1,1-dioxide

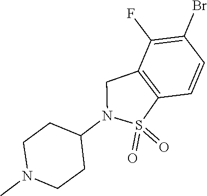 [SMILES: CN1CCC(N2Cc3c(ccc(Br)c3F)S2(=O)=O)CC1]

A mixture of 4-bromo-1-methylpiperidine (1 g, 5.62 mmol), Cs₂CO₃ (1.1 g, 3.36 mmol) and 5-bromo-4-fluoro-2,3-dihydrobenzo[d]isothiazole 1,1-dioxide (0.3 g, 1.12 mmol) in DMF (10 mL) was stirred at 50° C. for 12 hr. The mixture was quenched with water (30 mL). The mixture was extracted by DCM (20 mL×3). The DCM layer was concentrated under reduced pressure. The residue was purified by silica gel column chromatography, eluting with PE/EtOAc (10: 1~5:1) to afford the product (0.07 g, 17.1%). LC-MS (ESI): m/z [M+Na]⁺=385.3.

### Step 2: cis-3-(3-((4-fluoro-2-(1-methylpiperidin-4-yl)-1,1-dioxido-2,3-dihydrobenzo[d]isothiazol-5-yl)amino-1H-pyrazol-5-yl)cyclopentyl isopropylcarbamate

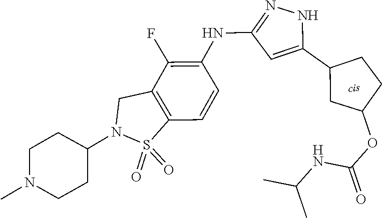 [SMILES: CC(C)NC(=O)OC1CCC(c2cc(Nc3ccc4c(c3F)CN(C3CCN(C)CC3)S4(=O)=O)n[nH]2)C1]

The titled compound was synthesized in the procedures similar to Example 34, step 3 to step 4. ¹H NMR (500 MHz, DMSO-d₆) δ 11.99 (s, 1H), 8.77 (s, 1H), 8.27 (s, 1H), 7.51 (d, J=8.6 Hz, 1H), 6.94 (d, J=7.5 Hz, 1H), 5.83 (s, 1H), 5.00 (s, 1H), 4.50 (s, 2H), 3.62-3.54 (m, 1H), 3.45-3.38 (m, 1H), 3.10-3.01 (m, 1H), 2.86 (d, J=11.5 Hz, 2H), 2.48-2.43 (m, 1H), 2.22 (s, 3H), 2.15-1.99 (m, 3H), 1.97-1.83 (m, 5H), 1.78-1.55 (m, 3H), 1.03 (d, J=6.1 Hz, 6H). LC-MS (ESI): m/z [M+H]⁺=535.3.

### Example 63: cis-3-(3-((2-(1-benzylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

### Step 1: tert-butyl 4-(5-bromo-4-fluoro-1,1-dioxidobenzo[d]isothiazol-2 (3H)-yl) piperidine-1-carboxylate

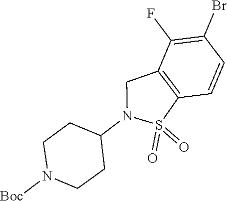 [SMILES: CC(C)(C)OC(=O)N1CCC(N2Cc3c(ccc(Br)c3F)S2(=O)=O)CC1]

A mixture of tert-butyl 4-hydroxypiperidine-1-carboxylate (1 g, 5.01 mmol), CMBP (2.7 g, 11.2 mmol) and 5-bromo-4-fluoro-2,3-dihydrobenzo[d]isothiazole 1,1-dioxide (1 g, 3.76 mmol) in toluene (20 mL) was stirred at 95° C. under N₂ for 12 hr. The mixture was concentrated under reduced pressure. The residue was purified by silica gel column chromatography, eluting with PE/EtOAc (9: 1~3:1) to afford the product (0.7 g, 41.7%). LC-MS (ESI): m/z [M+H]⁺=449.

### Step 2:5-bromo-4-fluoro-2-(piperidin-4-yl)-2,3-dihydrobenzo[d]isothiazole 1,1-dioxide

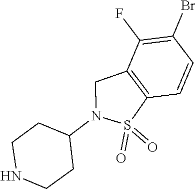 [SMILES: O=S1(=O)c2ccc(Br)c(F)c2CN1C1CCNCC1]

To a solution of tert-butyl 4-(5-bromo-4-fluoro-1,1-dioxidobenzo[d]isothiazol-2 (3H)-yl) piperidine-1-carboxylate (0.55 g, 1.22 mmol) in DCM (10 mL), TFA (5 mL) was added at 25° C. Then the mixture was stirred at 25° C. for 0.5 hr. The mixture was quenched with saturated aq. NaHCO₃ (20 mL). The mixture was extracted by DCM (30 mL×3). The DCM layer was concentrated under reduced pressure to afford the product, which was used for next step without purification. (0.42 g, 98.4%). LC-MS (ESI): m/z [M+H]⁺=349.

### Step 3:2-(1-benzylpiperidin-4-yl)-5-bromo-4-fluoro-2,3-dihydrobenzo[d]isothiazole 1,1-dioxide

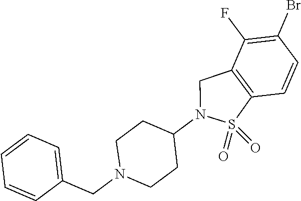 [SMILES: O=S1(=O)c2ccc(Br)c(F)c2CN1C1CCN(Cc2ccccc2)CC1]

To a mixture of 5-bromo-4-fluoro-2-(piperidin-4-yl)-2,3-dihydrobenzo[d]isothiazole 1,1-dioxide (0.18 g, 0.52 mmol) and benzaldehyde (0.11 g, 1.04 mmol) in DCM (10 mL) was added Sodium triacetoxyborohydride (0.22 g, 1.04 mmol) and 1 drop of HOAc. The mixture was stirred at 25° C. for 2 hr. The mixture was concentrated under reduced pressure. The residue was purified by silica gel column chromatography, eluting with PE/EtOAc (10: 1~5:1) to afford the product (0.1 g, 44.2%). LC-MS (ESI): m/z [M+H]⁺=439.

### Step 4: cis-3-(3-((2-(1-benzylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

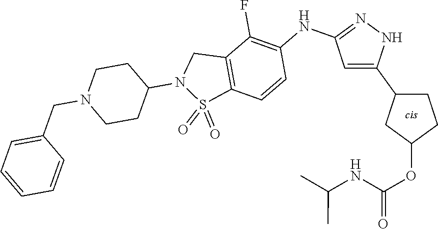 [SMILES: CC(C)NC(=O)OC1CCC(c2cc(Nc3ccc4c(c3F)CN(C3CCN(Cc5ccccc5)CC3)S4(=O)=O)n[nH]2)C1]

The titled compound was synthesized in the procedures similar to Example 34. ¹H NMR (500 MHz, DMSO-d₆) δ 11.99 (s, 1H), 8.76 (s, 1H), 8.29 (s, 1H), 7.50 (d, J=8.6 Hz, 1H), 7.37-7.29 (m, 4H), 7.25 (t, J=6.3 Hz, 1H), 6.94 (d, J=6.8 Hz, 1H), 5.83 (s, 1H), 5.00 (s, 1H), 4.51 (s, 2H), 3.62-3.53 (m, 1H), 3.50-3.41 (m, 3H), 3.11-3.02 (m, 1H), 2.86 (d, J=11.5 Hz, 2H), 2.48-2.43 (m, 1H), 2.13-1.98 (m, 3H), 1.98-1.83 (m, 5H), 1.78-1.55 (m, 3H), 1.03 (d, J=6.2 Hz, 6H). LC-MS (ESI): m/z [M+H]⁺=611.3.

### Example 64: cis-3-(3-((4-fluoro-2-(2-methoxyethyl)-1,1-dioxido-2,3-dihydrobenzo[d]isothiazol-5-yl)amino)-1H-pyrazol-5-yl)cyclopentyl isopropylcarbamate

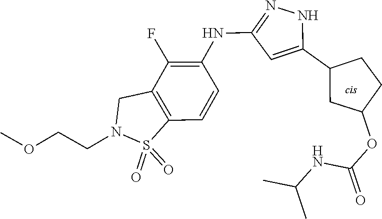 [SMILES: COCCN1Cc2c(ccc(Nc3cc(C4CCC(OC(=O)NC(C)C)C4)[nH]n3)c2F)S1(=O)=O]

The titled compound was synthesized in the procedures similar to Example 35. ¹H NMR (500 MHz, DMSO-d₆) δ 12.00 (s, 1H), 8.79 (s, 1H), 8.15-8.06 (m, 1H), 7.54 (d, J=5 Hz, 1H), 6.94 (d, J=5 Hz, 1H), 5.84 (s, 1H), 5.04-4.97 (m, 1H), 4.53 (s, 2H), 3.62 (t, J=5 Hz, 2H), 3.60-3.54 (m, 1H), 3.34-3.33 (m, 1H), 3.33-3.31 (m, 3H), 3.30 (s, 2H), 2.48-2.44 (m, 1H), 2.08-1.99 (m, 1H), 1.97-1.86 (m, 1H), 1.79-1.66 (m, 2H), 1.64-1.55 (m, 1H), 1.09-0.98 (m, 6H). LC-MS (ESI): m/z [M+H]⁺=496.2.

### Example 65: cis-3-(3-((4-fluoro-2-isopropyl-1,1-dioxido-2,3-dihydrobenzo[d]isothiazol-5-yl)amino)-1H-pyrazol-5-yl)cyclopentyl isopropylcarbamate

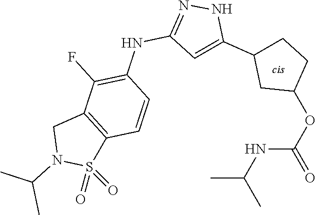 [SMILES: CC(C)NC(=O)OC1CCC(c2cc(Nc3ccc4c(c3F)CN(C(C)C)S4(=O)=O)n[nH]2)C1]

The titled compound was synthesized in the procedures similar to Example 35. ¹H NMR (500 MHz, DMSO-d₆) δ 11.99 (s, 1H), 8.76 (s, 1H), 8.36-8.24 (m, 1H), 7.52-7.47 (m, 1H), 6.97-6.91 (m, 1H), 5.83 (s, 1H), 5.05-4.96 (m, 1H), 4.47 (s, 2H), 3.90-3.81 (m, 1H), 3.63-3.53 (m, 1H), 3.12-3.01 (m, 1H), 2.45-2.40 (m, 1H), 2.07-1.98 (m, 1H), 1.96-1.86 (m, 1H), 1.78-1.65 (m, 2H), 1.64-1.55 (m, 1H), 1.34-1.26 (m, 6H), 1.08-0.99 (m, 6H). LC-MS (ESI): m/z [M+H]⁺=480.2.

### Example 66: cis-3-(3-((4-fluoro-1,1-dioxido-2-(2,2,2-trifluoroethyl)-2,3-dihydrobenzo[d]isothiazol-5-yl)amino)-1H-pyrazol-5-yl)cyclopentyl isopropylcarbamate

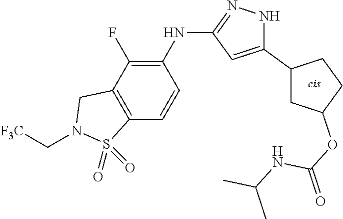 [SMILES: CC(C)NC(=O)OC1CCC(c2cc(Nc3ccc4c(c3F)CN(CC(F)(F)F)S4(=O)=O)n[nH]2)C1]

The titled compound was synthesized in the procedures similar to Example 35. ¹H NMR (500 MHz, DMSO-d₆) δ 12.03 (s, 1H), 8.87 (s, 1H), 8.38-8.26 (m, 1H), 7.62 (d, J=5 Hz, 1H), 6.94 (d, J=5 Hz, 1H), 5.85 (s, 1H), 5.06-4.96 (m, 1H), 4.73 (s, 2H), 4.23-4.09 (m, 2H), 3.66-3.52 (m, 1H), 3.14-3.00 (m, 1H), 2.48-2.44 (m, 1H), 2.09-1.99 (m, 1H), 1.97-1.86 (m, 1H), 1.80-1.65 (m, 2H), 1.64-1.55 (m, 1H), 1.11-0.95 (m, 6H). LC-MS (ESI): m/z [M+H]⁺=520.2.

### Example 67: cis-3-(3-((2-cyclobutyl-4-fluoro-1,1-dioxido-2,3-dihydrobenzo[d]isothiazol-5-yl)amino)-1H-pyrazol-5-yl)cyclopentyl isopropylcarbamate

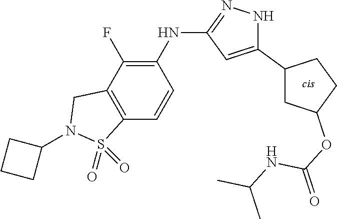 [SMILES: CC(C)NC(=O)OC1CCC(c2cc(Nc3ccc4c(c3F)CN(C3CCC3)S4(=O)=O)n[nH]2)C1]

The titled compound was synthesized in the procedures similar to Example 35. ¹H NMR (500 MHz, DMSO-d₆) δ 11.93 (s, 1H), 8.71 (s, 1H), 8.22-8.19 (m, 1H), 7.43 (d, J=10 Hz, 1H), 6.87 (d, J=5 Hz, 1H), 5.76 (s, 1H), 4.97-4.89 (m, 1H), 4.43 (s, 2H), 4.0-3.92 (m, 1H), 3.55-3.47 (m, 1H), 3.06-2.95 (m, 1H), 2.41-2.36 (m, 1H), 2.34-2.24 (m, 2H), 2.14-2.05 (m, 2H), 2.00-1.91 (m, 1H), 1.89-1.79 (m, 1H), 1.76-1.60 (m, 4H), 1.57-1.48 (m, 1H), 1.02-0.89 (m, 6H). LC-MS (ESI): m/z [M+H]⁺=492.2.

### Example 68: cis-3-(3-((2-cyclopentyl-4-fluoro-1,1-dioxido-2,3-dihydrobenzo[d]isothiazol-5-yl)amino-1H-pyrazol-5-yl)cyclopentyl isopropylcarbamate

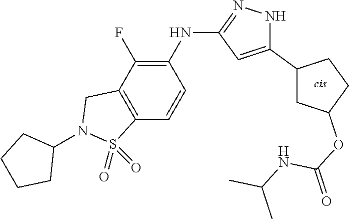 [SMILES: CC(C)NC(=O)OC1CCC(c2cc(Nc3ccc4c(c3F)CN(C3CCCC3)S4(=O)=O)n[nH]2)C1]

The titled compound was synthesized in the procedures similar to Example 35. ¹H NMR (500 MHz, DMSO-d₆) δ 11.92 (s, 1H), 8.70 (s, 1H), 8.22 (s, 1H), 7.43 (d, J=8.6 Hz, 1H), 6.87 (d, J=7.3 Hz, 1H), 5.76 (s, 1H), 4.93 (s, 1H), 4.40 (s, 2H), 3.68 (p, J=7.4 Hz, 1H), 3.58-3.48 (m, 1H), 3.03-2.94 (m, 1H), 2.40-2.33 (m, 1H), 1.99-1.93 (m, 1H), 1.92-1.81 (m, 3H), 1.80-1.75 (m, 2H), 1.70-1.64 (m, 4H), 1.58-1.47 (m, 3H), 1.01-0.91 (m, 6H). LC-MS (ESI): m/z [M+H]⁺=506.2.

### Example 69: cis-3-(3-((4-fluoro-1,1-dioxido-2-(tetrahydrofuran-3-yl)-2,3-dihydrobenzo[d]isothiazol-5-yl)amino)-1H-pyrazol-5-yl)cyclopentyl isopropylcarbamate

### Step 1:5-bromo-4-fluoro-2-(tetrahydrofuran-3-yl)-2,3-dihydrobenzo[d]isothiazole 1,1-dioxide

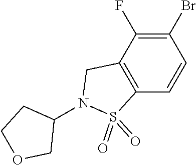 [SMILES: O=S1(=O)c2ccc(Br)c(F)c2CN1C1CCOC1]

To a solution of 5-bromo-4-fluoro-2,3-dihydrobenzo[d]isothiazole 1,1-dioxide (0.5 g, 1.88 mmol) in DMF (10 mL) was added NaH (150 mg, 3.76 mmol) in portions at 0° C. After stirring for 0.5 h, 3-iodotetrahydrofuran (560 mg, 2.82 mmol) was added. The resulting mixture was stirred for 16 h at 80° C. The mixture was quenched by addition of saturated NH₄Cl (aq.) (10 mL) and extracted with EtOAc (30 mL×2). The organic phase was washed with brine and concentrated under reduced pressure. The residue was purified by silica gel column chromatography, eluting with PE/EtOAc (2:1) to afford the product (100 mg, 16%), LC-MS (ESI): m/z [M+H]⁺=336.2.

### Step 2: cis-3-(3-((4-fluoro-1,1-dioxido-2-(tetrahydrofuran-3-yl)-2,3-dihydrobenzo[d]isothiazol-5-yl)amino)-1H-pyrazol-5-yl)cyclopentyl isopropylcarbamate

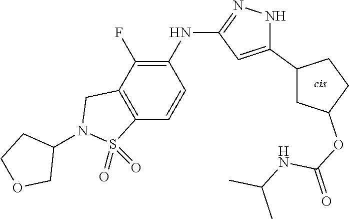 [SMILES: CC(C)NC(=O)OC1CCC(c2cc(Nc3ccc4c(c3F)CN(C3CCOC3)S4(=O)=O)n[nH]2)C1]

The titled compound was synthesized in the procedures similar to Example 34, step 5. ¹H NMR (500 MHz, DMSO-d₆) δ 12.00 (s, 1H), 8.80 (s, 1H), 8.30 (s, 1H), 7.54 (d, J=8.6 Hz, 1H), 6.95 (d, J=7.3 Hz, 1H), 5.84 (s, 1H), 5.07-4.94 (m, 1H), 4.53 (q, J=14.1 Hz, 2H), 4.25-4.15 (m, 1H), 3.97-3.90 (m, 2H), 3.80 (dd, J=9.6, 6.3 Hz, 1H), 3.68 (dd, J=15.3, 8.1 Hz, 1H), 3.62-3.54 (m, 1H), 3.11-3.02 (m, 1H), 2.48-2.43 (m, 1H), 2.29-2.14 (m, 2H), 2.08-1.97 (m, 1H), 1.95-1.86 (m, 1H), 1.78-1.66 (m, 2H), 1.64-1.55 (m, 1H), 1.09-0.97 (m, 6H). LC-MS (ESI): m/z [M+H]⁺=508.4.

### Example 70: cis-3-(3-((4-fluoro-2-(1-methylpyrrolidin-3-yl)-1,1-dioxido-2,3-dihydrobenzo[d]isothiazol-5-yl)amino-1H-pyrazol-5-yl)cyclopentyl isopropylcarbamate

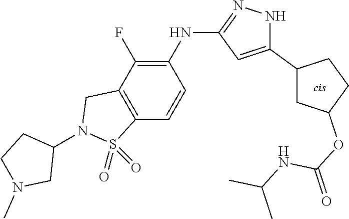 [SMILES: CC(C)NC(=O)OC1CCC(c2cc(Nc3ccc4c(c3F)CN(C3CCN(C)C3)S4(=O)=O)n[nH]2)C1]

The titled compound was synthesized in the procedures similar to Example 63. ¹H NMR (500 MHz, DMSO-d₆)) δ 11.99 (s, 1H), 8.79 (s, 1H), 8.29 (s, 1H), 7.52 (d, J=8.6 Hz, 1H), 6.94 (d, J=6.9 Hz, 1H), 5.83 (s, 1H), 5.00 (s, 1H), 4.61-4.48 (m, 2H), 4.17 (s, 1H), 3.63-3.54 (m, 1H), 3.12-3.03 (m, 1H), 2.88-2.73 (m, 2H), 2.64 (s, 1H), 2.48-2.43 (m, 1H), 2.36 (s, 1H), 2.31 (s, 3H), 2.20-2.13 (m, 1H), 2.08-1.98 (m, 2H), 1.95-1.86 (m, 1H), 1.78-1.55 (m, 3H), 1.03 (d, J=5.9 Hz, 6H). LC-MS (ESI): m/z [M+H]⁺=521.3.

### Example 71: cis-3-(3-((2-(2,3-dihydroxypropyl)-4-fluoro-1,1-dioxido-2,3-dihydrobenzo[d]isothiazol-5-yl)amino)-1H-pyrazol-5-yl)cyclopentyl isopropylcarbamate

### Step 1: cis-3-(3-((2-((2,2-dimethyl-1,3-dioxolan-4-yl)methyl)-4-fluoro-1,1-dioxido-2,3-dihydrobenzo[d]isothiazol-5-yl)amino)-1H-pyrazol-5-yl)cyclopentyl isopropylcarbamate

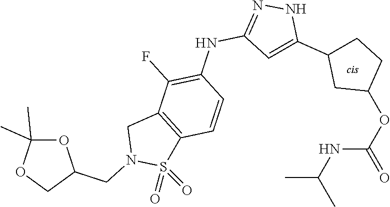 [SMILES: CC(C)NC(=O)OC1CCC(c2cc(Nc3ccc4c(c3F)CN(CC3COC(C)(C)O3)S4(=O)=O)n[nH]2)C1]

To a solution of cis-3-(3-((4-fluoro-1,1-dioxido-2,3-dihydrobenzo[d]isothiazol-5-yl)amino)-1H-pyrazol-5-yl)cyclopentyl isopropylcarbamate (50 mg, 0.11 mmol) in toluene (10 mL) was added (2,2-dimethyl-1,3-dioxolan-4-yl) methanol (29 mg,0.22 mmol) and CMBP (106 mg,0.44 mmol). The mixture was stirred for 12 h at 100° C. The solvent was removed under vacuum, and the residue was purified by silica gel column chromatography, eluting with EtOAc (100%) to afford the product (70 mg, 95%). LC-MS (ESI): m/z [M+H]⁺=552.4.

### Step 2: cis-3-(3-((2-(2,3-dihydroxypropyl)-4-fluoro-1,1-dioxido-2,3-dihydrobenzo[d]isothiazol-5-yl)amino)-1H-pyrazol-5-yl)cyclopentyl isopropylcarbamate

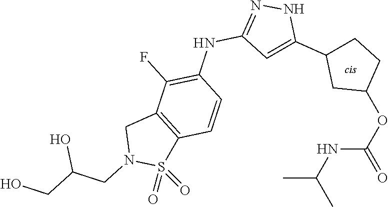 [SMILES: CC(C)NC(=O)OC1CCC(c2cc(Nc3ccc4c(c3F)CN(CC(O)CO)S4(=O)=O)n[nH]2)C1]

To a flask charged with cis-3-(3-((2-((2,2-dimethyl-1,3-dioxolan-4-yl)methyl)-4-fluoro-1,1-dioxido-2,3-dihydrobenzo[d]isothiazol-5-yl)amino)-1H-pyrazol-5-yl)cyclopentyl isopropylcarbamate (70 mg, 0.11 mmol) was added TFA (1 mL) and DCM (3 mL). The result solution was stirred at room temperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (28 mg, 50%). ¹H NMR (500 MHz, DMSO-d₆)) δ=12.00 (s, 1H), 8.77 (s, 1H), 8.28 (s, 1H), 7.54 (d, J=8.6, 1H), 6.94 (d, J=7.3, 1H), 5.83 (s, 1H), 5.00 (s, 1H), 4.61 (d, J=14.4, 1H), 4.51 (d, J=14.4, 1H), 4.42 (brs, 2H), 3.75 (s, 1H), 3.58 (s, 1H), 3.42 (s, 1H), 3.35-3.30 (m, 2H), 3.12-3.04 (m, 1H), 3.00 (s, 1H), 2.47-2.42 (m, 1H), 2.12-1.97 (m, 1H), 1.97-1.85 (m, 1H), 1.72-1.65 (m, 2H), 1.59 (m, 1H), 1.03 (m, 6H). LC-MS (ESI): m/z [M+H]⁺=512.4.

### Example 72: cis-3-(1-(2,3-dihydroxypropyl)-3-((2-(2,3-dihydroxypropyl)-4-fluoro-1,1-dioxido-2,3-dihydrobenzo[d]isothiazol-5-yl)amino)-1H-pyrazol-5-yl)cyclopentyl isopropylcarbamate

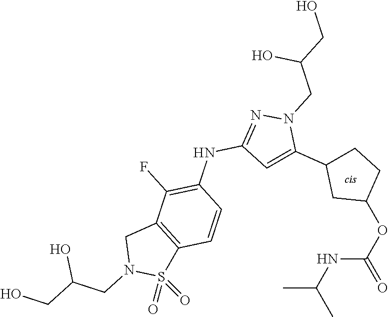 [SMILES: CC(C)NC(=O)OC1CCC(c2cc(Nc3ccc4c(c3F)CN(CC(O)CO)S4(=O)=O)nn2CC(O)CO)C1]

The titled compound was synthesized in the procedures similar to Example 71. ¹H NMR (500 MHz, DMSO-d₆) δ=8.67 (d, J=127.6, 1H), 8.27 (t, J=8.0, 0.5H), 7.54 (dd, J=14.5, 8.6, 1H), 7.10 (t, J=6.7, 0.5H), 6.98-6.87 (m, 1H), 5.94 (d, J=94.5, 1H), 5.22 (brs, 4H), 4.99 (s, 1H), 4.62 (dd, J=14.5, 8.2, 1H), 4.52 (dd, J=14.5, 9.3, 1H), 4.05 (m, 1H), 3.94-3.73 (m, 3H), 3.62-3.53 (m, 1H), 3.45-3.28 (m, 5H), 3.09 (m, 2H), 2.54 (m, 0.5H), 2.44-2.35 (m, 0.5H), 2.00 (m, 1H), 1.94-1.82 (m, 1H), 1.74 (m, 2H), 1.66-1.45 (m, 1H), 1.08-0.97 (m, 6H). LC-MS (ESI): m/z [M+H]⁺=586.5.

### Example 73: cis-3-(3-((2-(3-(dimethylamino) propyl)-4-fluoro-1,1-dioxido-2,3-dihydrobenzo[d]isothiazol-5-yl)amino)-1H-pyrazol-5-yl)cyclopentyl isopropylcarbamate

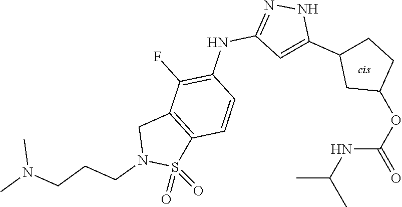 [SMILES: CC(C)NC(=O)OC1CCC(c2cc(Nc3ccc4c(c3F)CN(CCCN(C)C)S4(=O)=O)n[nH]2)C1]

The titled compound was synthesized in the procedures similar to Example 35. ¹H NMR (500 MHz, DMSO-d₆) δ 11.97 (s, 1H), 8.78 (s, 1H), 8.28 (t, J=7.6 Hz, 1H), 7.53 (d, J=8.6 Hz, 1H), 6.94 (d, J=7.3 Hz, 1H), 5.83 (s, 1H), 5.00 (s, 1H), 4.48 (s, 2H), 3.67-3.51 (m, 2H), 3.19 (t, J=7.0 Hz, 2H), 3.11-3.01 (m, 1H), 2.50-2.46 (m, 2H), 2.28 (s, 6H), 2.08-2.00 (m, 1H), 1.96-1.88 (m, 1H), 1.86-1.80 (m, 2H), 1.76-1.66 (m, 2H), 1.64-1.59 (m, 1H), 1.11-0.96 (m, 6H). LC-MS (ESI): m/z [M+H]⁺=523.2.

### Example 74: cis-3-(3-((2-(2-(dimethylamino)ethyl)-4-fluoro-1,1-dioxido-2,3-dihydrobenzo[d]isothiazol-5-yl)amino-1H-pyrazol-5-yl)cyclopentyl isopropylcarbamate

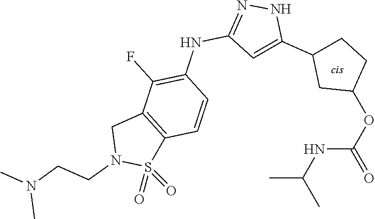 [SMILES: CC(C)NC(=O)OC1CCC(c2cc(Nc3ccc4c(c3F)CN(CCN(C)C)S4(=O)=O)n[nH]2)C1]

The titled compound was synthesized in the procedures similar to Example 35. ¹H NMR (500 MHz, DMSO-d₆) δ 12.01 (s, 1H), 8.84 (s, 1H), 8.32 (s, 1H), 7.59 (d, J=8.7 Hz, 1H), 6.93 (d, J=7.4 Hz, 1H), 5.84 (s, 1H), 5.00 (s, 1H), 4.56 (s, 2H), 3.64-3.54 (m, 1H), 3.46 (s, 2H), 3.23 (s, 1H), 3.11-3.01 (m, 2H), 2.68 (s, 6H), 2.48-2.40 (m, 1H), 2.06-1.98 (m, 1H), 1.96-1.85 (m, 1H), 1.79-1.66 (m, 2H), 1.63-1.53 (m, 1H), 1.03 (d, J=6.1 Hz, 6H). LC-MS (ESI): m/z [M+H]⁺=509.2.

### Example 75: cis-3-(3-((2-benzyl-4-fluoro-1,1-dioxido-2,3-dihydrobenzo[d]isothiazol-5-yl)amino)-1H-pyrazol-5-yl)cyclopentyl isopropylcarbamate

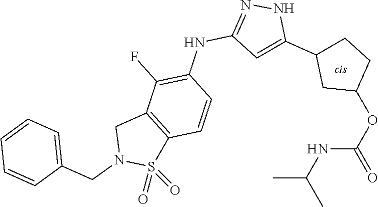 [SMILES: CC(C)NC(=O)OC1CCC(c2cc(Nc3ccc4c(c3F)CN(Cc3ccccc3)S4(=O)=O)n[nH]2)C1]

The titled compound was synthesized in the procedures similar to Example 35. ¹H NMR (500 MHz, DMSO-d₆) δ 12.00 (s, 1H), 8.78 (s, 1H), 8.32 (t, J=8.0 Hz, 1H), 7.59 (d, J=8.7 Hz, 1H), 7.47-7.38 (m, 4H), 7.36-7.32 (m, 1H), 6.94 (d, J=7.3 Hz, 1H), 5.82 (s, 1H), 5.00 (s, 1H), 4.35 (d, J=15.5 Hz, 4H), 3.62-3.52 (m, 1H), 3.15-2.94 (m, 1H), 2.48-2.40 (m, 1H), 2.06-1.99 (m, 1H), 1.96-1.85 (m, 1H), 1.79-1.66 (m, 2H), 1.63-1.53 (m, 1H), 1.08-0.97 (m, 6H). LC-MS (ESI): m/z [M+H]⁺=528.2.

### Example 76: cis-3-(3-((4-fluoro-2-(2-hydroxy-2-methylpropyl)-1,1-dioxido-2,3-dihydrobenzo[d]isothiazol-5-yl)amino)-1H-pyrazol-5-yl)cyclopentyl isopropylcarbamate

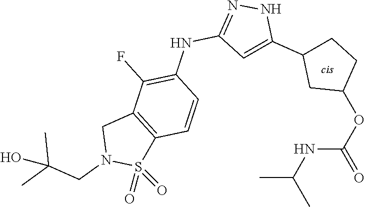 [SMILES: CC(C)NC(=O)OC1CCC(c2cc(Nc3ccc4c(c3F)CN(CC(C)(C)O)S4(=O)=O)n[nH]2)C1]

The titled compound was synthesized in the procedures similar to Example 63. ¹H NMR (500 MHz, DMSO-d₆) δ 11.99 (s, 1H), 8.77 (s, 1H), 8.36-8.24 (m, 1H), 7.58-7.50 (m, 1H), 6.98-6.89 (m, 1H), 5.83 (s, 1H), 5.04-4.94 (m, 1H), 4.67-4.55 (m, 3H), 3.63-3.51 (m, 1H), 3.11.2.99 (m, 3H), 2.43-2.40 (m, 1H), 2.08-1.96 (m, 1H), 1.95-1.84 (m, 1H), 1.78-1.66 (m, 2H), 1.64-1.53 (m, 1H), 1.23-1.12 (m, 6H), 1.08-1.94 (m, 6H). LC-MS (ESI): m/z [M+H]⁺=510.2.

### Example 77: cis-3-(3-((4-fluoro-2-(1-(methylsulfonyl) piperidin-4-yl)-1,1-dioxido-2,3-dihydrobenzo[d]isothiazol-5-yl)amino-1H-pyrazol-5-yl)cyclopentyl isopropylcarbamate

### Step 1:5-bromo-4-fluoro-2-(1-(methylsulfonyl) piperidin-4-yl)-2,3-dihydrobenzo[d]isothiazole 1,1-dioxide

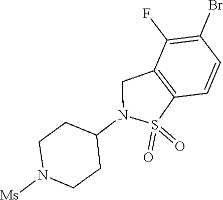 [SMILES: CS(=O)(=O)N1CCC(N2Cc3c(ccc(Br)c3F)S2(=O)=O)CC1]

To a solution of tert-butyl 4-(5-bromo-4-fluoro-1,1-dioxidobenzo[d]isothiazol-2 (3H)-yl) piperidine-1-carboxylate (0.18 g, 0.52 mmol) and DIEA (0.33 g, 2.56 mmol) in DCM (10 mL), MsCl (0.07 g, 0.61 mmol) was added at 0° C. Then the mixture was stirred at 25° C. for 1 hr. The mixture was diluted by DCM (20 mL) and washed with brine (30 mL×3). The DCM layer was concentrated under reduced pressure. The residue was purified by silica gel column chromatography, eluting with PE/EtOAc (10: 1~4:1) to afford the product (0.1 g, 45.4%). LC-MS (ESI): m/z [M+H]⁺=427.3.

### Step 2: cis-3-(3-((4-fluoro-2-(1-(methylsulfonyl) piperidin-4-yl)-1,1-dioxido-2,3-dihydrobenzo[d]isothiazol-5-yl)amino)-1H-pyrazol-5-yl)cyclopentyl isopropylcarbamate

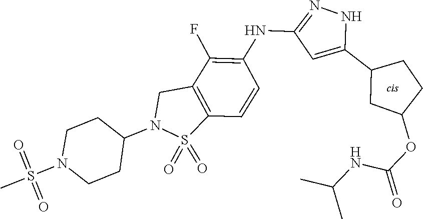 [SMILES: CC(C)NC(=O)OC1CCC(c2cc(Nc3ccc4c(c3F)CN(C3CCN(S(C)(=O)=O)CC3)S4(=O)=O)n[nH]2)C1]

The titled compound was synthesized in the procedures similar to Example 34, step 5. ¹H NMR (500 MHz, DMSO-d₆) δ 11.99 (s, 1H), 8.78 (s, 1H), 8.30 (t, J=7.8 Hz, 1H), 7.52 (d, J=8.7 Hz, 1H), 6.93 (d, J=6.4 Hz, 1H), 5.83 (s, 1H), 5.00 (s, 1H), 4.54 (s, 2H), 3.68-3.55 (m, 4H), 3.12-3.03 (m, 1H), 2.98-2.93 (m, 2H), 2.89 (s, 3H), 2.46-2.43 (m, 1H), 2.07-1.88 (m, 6H), 1.75-1.55 (m, 3H), 1.03 (d, J=6.0 Hz, 6H). LC-MS (ESI): m/z [M+H]⁺=599.3.

### Example 78: cis-3-(3-((2-(1-acetylpiperidin-4-yl)-4-fluoro-1,1-dioxido-2,3-dihydrobenzo[d]isothiazol-5-yl)amino)-1H-pyrazol-5-yl)cyclopentyl isopropylcarbamate

### Step 1:1-(4-(5-bromo-4-fluoro-1,1-dioxidobenzo[d]isothiazol-2 (3H)-yl) piperidin-1-yl)ethan-1-one

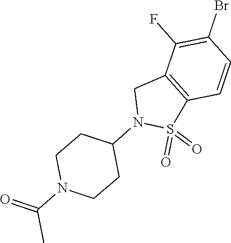 [SMILES: CC(=O)N1CCC(N2Cc3c(ccc(Br)c3F)S2(=O)=O)CC1]

To a solution of tert-butyl 4-(5-bromo-4-fluoro-1,1-dioxidobenzo[d]isothiazol-2 (3H)-yl) piperidine-1-carboxylate (0.18 g, 0.52 mmol) and DIEA (0.33 g, 2.56 mmol) in DCM (10 mL), AcCl (0.049 g, 0.62 mmol) was added at 0° C. Then the mixture was stirred at 25° C. for 1 hr. The mixture was diluted by DCM (20 mL) and washed with brine (30 mL×3). The DCM layer was concentrated under reduced pressure. The residue was purified by silica gel column chromatography, eluting with PE/EtOAc (5: 1~3:1) to afford the product (0.1 g, 49.7%). LC-MS (ESI): m/z [M+H]⁺=391.5.

### Step 2: cis-3-(3-((2-(1-acetylpiperidin-4-yl)-4-fluoro-1,1-dioxido-2,3-dihydrobenzo[d]isothiazol-5-yl)amino-1H-pyrazol-5-yl)cyclopentyl isopropylcarbamate

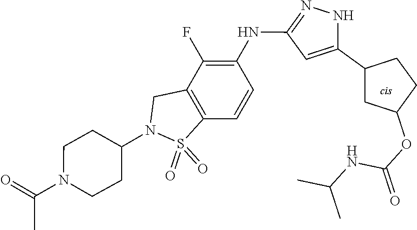 [SMILES: CC(=O)N1CCC(N2Cc3c(ccc(Nc4cc(C5CCC(OC(=O)NC(C)C)C5)[nH]n4)c3F)S2(=O)=O)CC1]

The titled compound was synthesized in the procedures similar to Example 34, step 5. ¹H NMR (500 MHz, DMSO-d₆) δ 11.99 (s, 1H), 8.76 (s, 1H), 8.29 (s, 1H), 7.51 (d, J=8.6 Hz, 1H), 6.93 (d, J=6.9 Hz, 1H), 5.83 (s, 1H), 5.00 (s, 1H), 4.51 (s, 2H), 4.35 (d, J=13.5 Hz, 1H), 3.86 (d, J=13.5 Hz, 1H), 3.74 (t, J=10.9 Hz, 1H), 3.62-3.54 (m, 1H), 3.20 (t, J=11.6 Hz, 1H), 3.10-3.02 (m, 1H), 2.75 (t, J=11.6 Hz, 1H), 2.48-2.43 (m, 1H), 2.05-1.98 (m, 4H), 1.96-1.80 (m, 4H), 1.77-1.55 (m, 4H), 1.03 (d, J=6.0 Hz, 6H). LC-MS (ESI): m/z [M+H]⁺=563.3.

### Example 79: cis-3-(3-((4-fluoro-1,1-dioxido-2-(2-oxopyrrolidin-3-yl)-2,3-dihydrobenzo[d]isothiazol-5-yl)amino)-1H-pyrazol-5-yl)cyclopentyl isopropylcarbamate

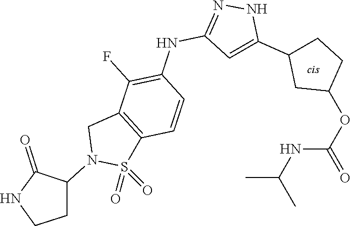 [SMILES: CC(C)NC(=O)OC1CCC(c2cc(Nc3ccc4c(c3F)CN(C3CCNC3=O)S4(=O)=O)n[nH]2)C1]

The titled compound was synthesized in the procedures similar to Example 63. ¹H NMR (500 MHz, DMSO-d₆) δ 12.00 (s, 1H), 8.80 (s, 1H), 8.31 (s, 1H), 8.13 (d, J=7.8 Hz, 1H), 7.55 (d, J=8.7 Hz, 1H), 6.94 (d, J=7.2 Hz, 1H), 5.84 (s, 1H), 5.00 (s, 1H), 4.71 (s, 1H), 4.37 (dd, J=23.3, 11.9 Hz, 2H), 3.62-3.55 (m, 1H), 3.28-3.21 (m, 2H), 3.09-3.02 (m, 1H), 2.48-2.43 (m, 1H), 2.38-2.25 (m, 2H), 2.08-1.85 (m, 2H), 1.78-1.55 (m, 3H), 1.03 (d, J=6.2 Hz, 6H). LC-MS (ESI): m/z [M+H]⁺=521.2

### Example 80: cis-3-(3-((2-(1-acetylpyrrolidin-3-yl)-4-fluoro-1,1-dioxido-2,3-dihydrobenzo[d]isothiazol-5-yl)amino)-1H-pyrazol-5-yl)cyclopentyl isopropylcarbamate

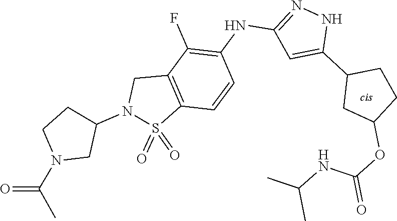 [SMILES: CC(=O)N1CCC(N2Cc3c(ccc(Nc4cc(C5CCC(OC(=O)NC(C)C)C5)[nH]n4)c3F)S2(=O)=O)C1]

The titled compound was synthesized in the procedures similar to Example 63. ¹H NMR (500 MHz, DMSO-d₆) δ 12.01 (s, 1H), 8.81 (s, 1H), 8.31 (s, 1H), 7.59-7.51 (m, 1H), 6.94 (d, J=6.8 Hz, 1H), 5.84 (s, 1H), 5.00 (s, 1H), 4.60-4.53 (m, 2H), 4.20-3.98 (m, 1H), 3.78-3.46 (m, 4H), 3.29-3.25 (m, 1H), 3.10-3.02 (m, 1H), 2.46-2.44 (m, 1H), 2.33-2.27 (m, 1H), 2.20 (q, J=6.9 Hz, 1H), 2.05-2.00 (m, 1H), 1.96 (d, J=4.9 Hz, 3H), 1.93-1.86 (m, 1H), 1.76-1.55 (m, 3H), 1.03 (d, J=6.0 Hz, 6H). LC-MS (ESI): m/z [M+H]⁺=549.3.

### Example 81: cis-3-(3-((4-fluoro-1,1-dioxido-2-(2-oxopiperidin-4-yl)-2,3-dihydrobenzo[d]isothiazol-5-yl)amino)-1H-pyrazol-5-yl)cyclopentyl isopropylcarbamate

### Step 1:4-(5-bromo-4-fluoro-1,1-dioxidobenzo[d]isothiazol-2 (3H)-yl) piperidin-2-one

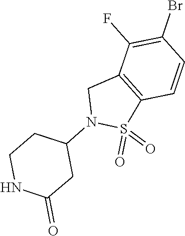 [SMILES: O=C1CC(N2Cc3c(ccc(Br)c3F)S2(=O)=O)CCN1]

A mixture of 5,6-dihydropyridin-2 (1H)-one (0.33 g, 3.4 mmol), Cs₂CO₃ (0.37 g, 1.14 mmol) and 5-bromo-4-fluoro-2,3-dihydrobenzo[d]isothiazole 1,1-dioxide (0.3 g, 1.12 mmol) in THF/water (10/10 mL) was stirred at 55° C. for 48 hr. The mixture was quenched with water (20 mL). The mixture was extracted by EA (20 mL×3). The EA layer was concentrated under reduced pressure. The residue was purified by silica gel column chromatography, eluting with PE/EtOAc (5: 1~3:1) then DCM/MeOH (10:1) to afford the product (0.077 g, 18.8%). LC-MS (ESI): m/z [M+H]⁺=363.2.

### Step 2: cis-3-(3-((4-fluoro-1,1-dioxido-2-(2-oxopiperidin-4-yl)-2,3-dihydrobenzo[d]isothiazol-5-yl)amino)-1H-pyrazol-5-yl)cyclopentyl isopropylcarbamate

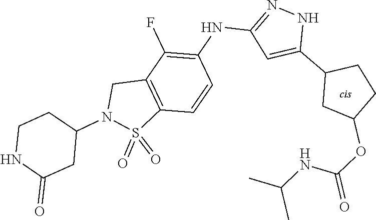 [SMILES: CC(C)NC(=O)OC1CCC(c2cc(Nc3ccc4c(c3F)CN(C3CCNC(=O)C3)S4(=O)=O)n[nH]2)C1]

The titled compound was synthesized in the procedures similar to Example 63.1H NMR (500 MHz, DMSO-d₆) δ 11.99 (s, 1H), 8.78 (s, 1H), 8.30 (t, J=8.0 Hz, 1H), 7.61 (s, 1H), 7.53 (d, J=8.6 Hz, 1H), 6.93 (d, J=6.6 Hz, 1H), 5.83 (s, 1H), 5.00 (s, 1H), 4.59 (d, J=14.2 Hz, 1H), 4.50 (d, J=14.0 Hz, 1H), 3.98-3.89 (m, 1H), 3.62-3.54 (m, 1H), 3.26-3.19 (m, 2H), 3.09-3.03 (m, 1H), 2.61 (dd, J=17.0, 9.9 Hz, 2H), 2.48-2.44 (m, 1H), 2.14-1.89 (m, 4H), 1.75-1.55 (m, 3H), 1.03 (d, J=6.0 Hz, 6H).

LC-MS (ESI): m/z [M+H]⁺=535.3.

### Example 82: cis-3-(3-((4-fluoro-1,1-dioxido-2-(6-oxopiperidin-3-yl)-2,3-dihydrobenzo[d]isothiazol-5-yl)amino)-1H-pyrazol-5-yl)cyclopentyl isopropylcarbamate

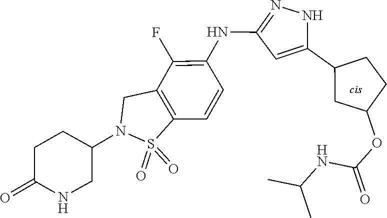 [SMILES: CC(C)NC(=O)OC1CCC(c2cc(Nc3ccc4c(c3F)CN(C3CCC(=O)NC3)S4(=O)=O)n[nH]2)C1]

The titled compound was synthesized in the procedures similar to Example 63. ¹H NMR (500 MHz, DMSO-d₆) δ 11.99 (s, 1H), 8.79 (s, 1H), 8.31 (t, J=7.9 Hz, 1H), 7.55-7.47 (m, 2H), 6.93 (d, J=6.7 Hz, 1H), 5.83 (s, 1H), 5.00 (s, 1H), 4.56 (d, J=27.0 Hz, 2H), 3.85-3.78 (m, 1H), 3.62-3.53 (m, 1H), 3.50-3.37 (m, 2H), 3.12-3.03 (m, 1H), 2.47-2.43 (m, 1H), 2.36-2.31 (m, 2H), 2.19-1.99 (m, 3H), 1.95-1.85 (m, 1H), 1.76-1.55 (m, 3H), 1.03 (d, J=5.9 Hz, 6H). LC-MS (ESI): m/z [M+H]⁺=535.3.

### Example 83: cis-3-(3-((4-fluoro-2-(1-methyl-6-oxopiperidin-3-yl)-1,1-dioxido-2,3-dihydrobenzo[d]isothiazol-5-yl)amino)-1H-pyrazol-5-yl)cyclopentyl isopropylcarbamate

### Step 1:5-(5-bromo-4-fluoro-1,1-dioxidobenzo[d]isothiazol-2 (3H)-yl) piperidin-2-one

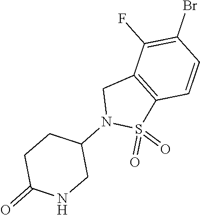 [SMILES: O=C1CCC(N2Cc3c(ccc(Br)c3F)S2(=O)=O)CN1]

To a stirring solution of 5-bromo-4-fluoro-2,3-dihydrobenzo[d]isothiazole 1,1-dioxide (250 mg, 1 mmol) in Toluene (20 mL) was added 5-hydroxypiperidin-2-one (265 mg, 2 mmol) CMBP (964 mg, 4 mmol). The solution was stirred for 90° C. for 16 h under N₂ atmosphere. The mixture was concentrated under reduced pressure. The residue was purified by silica gel column chromatography, eluting with PE/EtOAc (3:1) to afford the product (203 mg, 56%), [M+H]⁺=363.4.

### Step 2:5-(5-bromo-4-fluoro-1,1-dioxidobenzo[d]isothiazol-2 (3H)-yl)-1-methylpiperidin-2-one

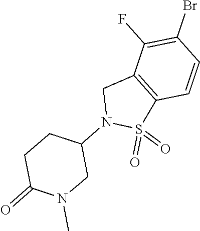 [SMILES: CN1CC(N2Cc3c(ccc(Br)c3F)S2(=O)=O)CCC1=O]

To a solution of 5-(5-bromo-4-fluoro-1,1-dioxidobenzo[d]isothiazol-2 (3H)-yl) piperidin-2-one (0.343 g, 0.95 mmol) in THF (10 mL), NaH (0.04 g, 1.0 mmol) was added at 0° C. The mixture was stirred for 0.5 hr. CH₃I (0.2 g, 1.43 mmol) was added and the mixture was stirred for 1 hr at 0° C. The mixture was quenched with saturated NH₄Cl (aq.) (10 mL). The mixture was extracted by EA (20 mL×3). The EA layer was concentrated under reduced pressure. The residue was purified by silica gel column chromatography, eluting with DCM/MeOH (20:1) to afford the product (0.19 g, 53.4%). LC-MS (ESI): m/z [M+H]⁺=377.4.

### Step 3: cis-3-(3-((4-fluoro-2-(1-methyl-6-oxopiperidin-3-yl)-1,1-dioxido-2,3-dihydrobenzo[d]isothiazol-5-yl)amino)-1H-pyrazol-5-yl)cyclopentyl isopropylcarbamate

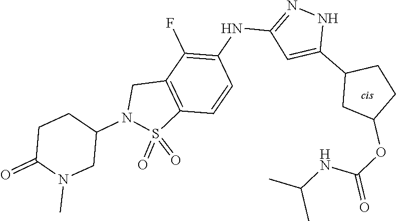 [SMILES: CC(C)NC(=O)OC1CCC(c2cc(Nc3ccc4c(c3F)CN(C3CCC(=O)N(C)C3)S4(=O)=O)n[nH]2)C1]

The titled compound was synthesized in the procedures similar to Example 34, step 5.1H NMR (500 MHz, DMSO-d₆) δ 12.00 (s, 1H), 8.81 (s, 1H), 8.31 (s, 1H), 7.54 (d, J=8.7 Hz, 1H), 6.94 (d, J=7.2 Hz, 1H), 5.84 (s, 1H), 5.00 (s, 1H), 4.57 (dd, J=40.3, 14.0 Hz, 2H), 3.97-3.89 (m, 1H), 3.66-3.51 (m, 3H), 3.12-3.02 (m, 1H), 2.83 (s, 3H), 2.46-2.35 (m, 3H), 2.85-1.85 (m, 4H), 1.78-1.55 (m, 3H), 1.03 (d, J=6.0 Hz, 6H). LC-MS (ESI): m/z [M+H]⁺=549.3.

### Example 84:3-(3-((4-fluoro-2-(3-hydroxycyclobutyl)-1,1-dioxido-2,3-dihydrobenzo[d]isothiazol-5-yl)amino)-1H-pyrazol-5-yl)cyclopentyl isopropylcarbamate

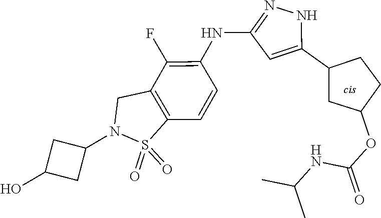 [SMILES: CC(C)NC(=O)OC1CCC(c2cc(Nc3ccc4c(c3F)CN(C3CC(O)C3)S4(=O)=O)n[nH]2)C1]

The titled compound was synthesized in the procedures similar to Example 63. ¹H NMR (500 MHz, DMSO-d₆) δ 11.99 (s, 1H), 8.78 (s, 1H), 8.29 (t, J=8.0 Hz, 1H), 7.50 (d, J=8.6 Hz, 1H), 6.94 (d, J=7.3 Hz, 1H), 5.83 (d, J=1.7 Hz, 1H), 5.18 (dd, J=17.4, 6.1 Hz, 1H), 4.99 (s, 1H), 4.46 (s, 2H), 4.32 (dd, J=11.7, 6.5 Hz, 1H), 4.11-3.97 (m, 1H), 3.68-3.52 (m, 1H), 3.16-2.97 (m, 1H), 2.70-2.57 (m, 2H), 2.50-2.44 (m, 1H), 2.15-2.10 (m, 2H), 2.06-1.94 (m, 1H), 1.95-1.85 (m, 1H), 1.77-1.65 (m, 2H), 1.65-1.55 (m, 1H), 1.03 (d, J=6.2 Hz, 6H). LC-MS (ESI): m/z [M+H]⁺=508.2.

### Example 85: cis-3-(3-((4-fluoro-2-(1-(methylsulfonyl) pyrrolidin-3-yl)-1,1-dioxido-2,3-dihydrobenzo[d]isothiazol-5-yl)amino)-1H-pyrazol-5-yl)cyclopentyl isopropylcarbamate

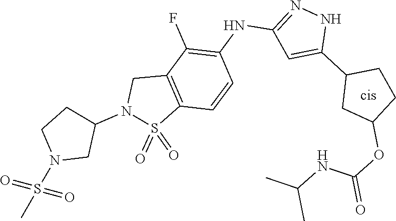 [SMILES: CC(C)NC(=O)OC1CCC(c2cc(Nc3ccc4c(c3F)CN(C3CCN(S(C)(=O)=O)C3)S4(=O)=O)n[nH]2)C1]

The titled compound was synthesized in the procedures similar to Example 35. ¹H NMR (500 MHz, DMSO-d₆) δ 12.01 (s, 1H), 8.83 (s, 1H), 8.32 (t, J=7.7 Hz, 1H), 7.56 (d, J=8.6 Hz, 1H), 6.94 (d, J=7.5 Hz, 1H), 5.84 (s, 1H), 4.99 (s, 1H), 4.59 (s, 2H), 4.18-4.11 (m, 1H), 3.62-3.54 (m, 2H), 3.52-3.42 (m, 2H), 3.35-3.31 (m, 1H), 3.10-3.03 (m, 1H), 2.96 (s, 3H), 2.47-2.42 (m, 1H), 2.28 (q, J=7.2 Hz, 2H), 2.06-1.88 (m, 2H), 1.77-1.55 (m, 3H), 1.03 (d, J=5.9 Hz, 6H). LC-MS (ESI): m/z [M+H]⁺=585.2.

### Example 86: cis-3-(3-((4-fluoro-1,1-dioxido-2-(2-oxopiperidin-3-yl)-2,3-dihydrobenzo[d]isothiazol-5-yl)amino)-1H-pyrazol-5-yl)cyclopentyl isopropylcarbamate

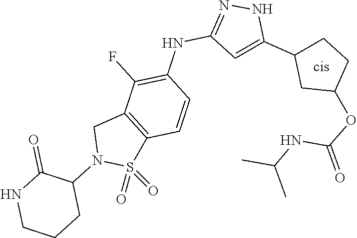 [SMILES: CC(C)NC(=O)OC1CCC(c2cc(Nc3ccc4c(c3F)CN(C3CCCNC3=O)S4(=O)=O)n[nH]2)C1]

The titled compound was synthesized in the procedures similar to Example 63. ¹H NMR (500 MHz, DMSO-d₆) δ 11.99 (s, 1H), 8.75 (s, 1H), 8.30 (s, 1H), 7.74 (s, 1H), 7.52 (d, J=8.7 Hz, 1H), 6.95 (d, J=7.5 Hz, 1H), 5.83 (s, 1H), 5.00 (s, 1H), 4.69 (d, J=14.1 Hz, 1H), 4.42 (d, J=14.0 Hz, 1H), 4.27-4.19 (m, 1H), 3.65-3.55 (m, 1H), 3.19-3.03 (m, 3H), 2.48-2.44 (m, 1H), 2.14-2.00 (m, 3H), 1.96-1.81 (m, 3H), 1.78-1.55 (m, 3H), 1.03 (d, J=5.8 Hz, 6H). LC-MS (ESI): m/z [M+H]⁺=535.3.

### Example 87: cis-3-(3-((2-(1-benzylpyrrolidin-3-yl)-4-fluoro-1,1-dioxido-2,3-dihydrobenzo[d]isothiazol-5-yl)amino)-1H-pyrazol-5-yl)cyclopentyl isopropylcarbamate

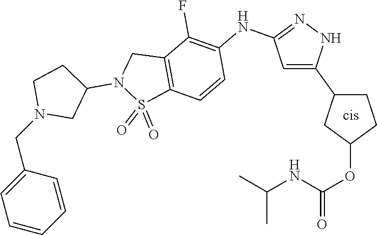 [SMILES: CC(C)NC(=O)OC1CCC(c2cc(Nc3ccc4c(c3F)CN(C3CCN(Cc5ccccc5)C3)S4(=O)=O)n[nH]2)C1]

The titled compound was synthesized in the procedures similar to Example 63. ¹H NMR (500 MHz, DMSO-d₆) δ 12.00 (s, 1H), 8.79 (s, 1H), 8.29 (s, 1H), 7.50 (d, J=8.6 Hz, 1H), 7.33 (d, J=4.3 Hz, 4H), 7.28-7.22 (m, 1H), 6.94 (d, J=6.6 Hz, 1H), 5.84 (s, 1H), 4.99 (s, 1H), 4.55 (dd, J=29.9, 14.2 Hz, 2H), 4.14 (s, 1H), 3.65-3.55 (m, 3H), 3.11-3.04 (m, 1H), 2.78-2.62 (m, 4H), 2.41-2.35 (m, 1H), 2.22-2.12 (m, 1H), 2.09-1.99 (m, 2H), 1.95-1.86 (m, 1H), 1.77-1.55 (m, 3H), 1.03 (d, J=6.0 Hz, 6H). LC-MS (ESI): m/z [M+H]⁺=597.3.

### Example 88: cis-3-(3-((4-fluoro-2-(1-methyl-2-oxopiperidin-3-yl)-1,1-dioxido-2,3-dihydrobenzo[d]isothiazol-5-yl)amino)-1H-pyrazol-5-yl)cyclopentyl isopropylcarbamate

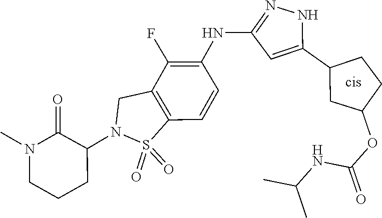 [SMILES: CC(C)NC(=O)OC1CCC(c2cc(Nc3ccc4c(c3F)CN(C3CCCN(C)C3=O)S4(=O)=O)n[nH]2)C1]

The titled compound was synthesized in the procedures similar to Example 63. ¹H NMR (500 MHz, DMSO-d₆) δ 11.99 (s, 1H), 8.75 (s, 1H), 8.30 (s, 1H), 7.51 (d, J=8.6 Hz, 1H), 6.95 (d, J=7.4 Hz, 1H), 5.83 (s, 1H), 4.99 (s, 1H), 4.66 (d, J=14.1 Hz, 1H), 4.39 (d, J=14.1 Hz, 1H), 4.29 (dd, J=11.6, 5.9 Hz, 1H), 3.61-3.53 (m, 1H), 3.29-3.24 (m, 2H), 3.10-3.02 (m, 1H), 2.83 (s, 3H), 2.39-2.33 (m, 1H), 2.20-2.00 (m, 3H), 1.98-1.85 (m, 3H), 1.78-1.55 (m, 3H), 1.03 (d, J=6.1 Hz, 6H). LC-MS (ESI): m/z [M+H]⁺=549.3.

### Example 89: cis-3-(3-((4-fluoro-2-(1-(methylsulfonyl) azetidin-3-yl)-1,1-dioxido-2,3-dihydrobenzo[d]isothiazol-5-yl)amino)-1H-pyrazol-5-yl)cyclopentyl isopropylcarbamate

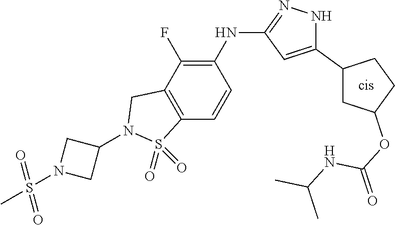 [SMILES: CC(C)NC(=O)OC1CCC(c2cc(Nc3ccc4c(c3F)CN(C3CN(S(C)(=O)=O)C3)S4(=O)=O)n[nH]2)C1]

The titled compound was synthesized in the procedures similar to Example 63. ¹H NMR (500 MHz, DMSO-d₆) δ=12.02 (s, 1H), 8.86 (s, 1H), 8.32 (s, 1H), 7.57 (d, J=8.7, 1H), 6.94 (d, J=7.5, 1H), 5.84 (s, 1H), 5.00 (s, 1H), 4.76 (s, 2H), 4.51 (m, 1H), 4.27 (m, 2H), 4.14 (m, 2H), 3.58 (m, 1H), 3.11 (s, 3H), 3.07 (m, 1H), 2.47 (m, 1H), 2.03 (m, 1H), 1.91 (m, 1H), 1.72 (m, 2H), 1.60 (m, 1H), 1.10-0.94 (m, 6H). LC-MS (ESI): m/z [M+H]⁺=571.3.

### Example 90: cis-3-(3-((2-(1-acetylazetidin-3-yl)-4-fluoro-1,1-dioxido-2,3-dihydrobenzo[d]isothiazol-5-yl)amino)-1H-pyrazol-5-yl)cyclopentyl isopropylcarbamate

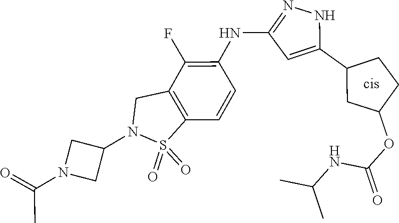 [SMILES: CC(=O)N1CC(N2Cc3c(ccc(Nc4cc(C5CCC(OC(=O)NC(C)C)C5)[nH]n4)c3F)S2(=O)=O)C1]

The titled compound was synthesized in the procedures similar to Example 63. ¹H NMR (500 MHz, DMSO-d₆) δ=12.01 (s, 1H), 8.85 (s, 1H), 8.32 (s, 1H), 7.56 (d, J=8.7, 1H), 6.94 (d, J=7.1, 1H), 5.84 (s, 1H), 5.00 (s, 1H), 4.70 (m, 2H), 4.43 (m, 2H), 4.38 (m, 1H), 4.19-4.07 (m, 2H), 3.58 (m, 1H), 3.13-2.98 (m, 1H), 2.48-2.42 (m, 1H), 2.02 (m, 1H), 1.95-1.85 (m, 1H), 1.79 (s, 3H), 1.72 (m, 2H), 1.60 (m, 1H), 1.09-0.96 (m, 6H). LC-MS (ESI): m/z [M+H]⁺=535.3.

### Example 91: cis-3-(3-((4-fluoro-2-(1-methylazetidin-3-yl)-1,1-dioxido-2,3-dihydrobenzo[d]isothiazol-5-yl)amino)-1H-pyrazol-5-yl)cyclopentyl isopropylcarbamate

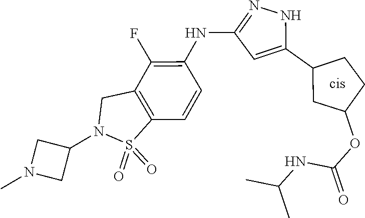 [SMILES: CC(C)NC(=O)OC1CCC(c2cc(Nc3ccc4c(c3F)CN(C3CN(C)C3)S4(=O)=O)n[nH]2)C1]

The titled compound was synthesized in the procedures similar to Example 63. ¹H NMR (500 MHz, DMSO-d₆) δ=12.00 (s, 1H), 8.81 (s, 1H), 8.29 (s, 1H), 7.53 (d, J=8.6, 1H), 6.94 (d, J=7.1, 1H), 5.84 (s, 1H), 4.99 (s, 1H), 4.61 (s, 2H), 4.11 (m, 1H), 3.64-3.54 (m, 1H), 3.50 (m, 2H), 3.34 (m, 2H), 3.13-2.97 (m, 1H), 2.46 (dm, 1H), 2.28 (s, 3H), 2.07-1.96 (m, 1H), 1.96-1.84 (m, 1H), 1.82-1.65 (m, 2H), 1.59 (m, 1H), 1.03 (m, 6H). LC-MS (ESI): m/z [M+H]⁺=507.5.

### Example 92: cis-3-(3-((2-(1-benzylazetidin-3-yl)-4-fluoro-1,1-dioxido-2,3-dihydrobenzo[d]isothiazol-5-yl)amino)-1H-pyrazol-5-yl)cyclopentyl isopropylcarbamate

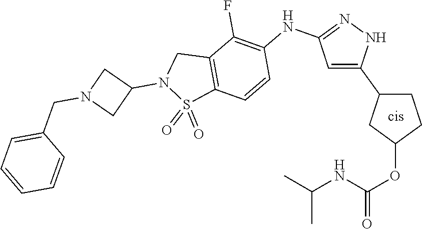 [SMILES: CC(C)NC(=O)OC1CCC(c2cc(Nc3ccc4c(c3F)CN(C3CN(Cc5ccccc5)C3)S4(=O)=O)n[nH]2)C1]

The titled compound was synthesized in the procedures similar to Example 63. ¹H NMR (500 MHz, DMSO-d₆) δ=12.00 (s, 1H), 8.81 (s, 1H), 8.30 (s, 1H), 7.53 (d, J=8.7, 1H), 7.35-7.27 (m, 4H), 7.25 (t, J=6.7, 1H), 6.94 (d, J=7.0, 1H), 5.84 (s, 1H), 5.00 (s, 1H), 4.62 (s, 2H), 4.16 (m, 1H), 3.64 (s, 2H), 3.58 (m, 1H), 3.49 (m, 2H), 3.38 (m, 2H), 3.15-2.96 (m, 1H), 2.45 (m, 1H), 2.08-1.98 (m, 1H), 1.98-1.84 (m, 1H), 1.80-1.64 (m, 2H), 1.60 (m, 1H), 1.03 (m, 6H). LC-MS (ESI): m/z [M+H]⁺=583.6.

### Example 93: cis-3-(3-((4-fluoro-2-(1-methyl-2-oxopyrrolidin-3-yl)-1,1-dioxido-2,3-dihydrobenzo[d]isothiazol-5-yl)amino)-1H-pyrazol-5-yl)cyclopentyl isopropylcarbamate

### Step 1:3-(5-bromo-4-fluoro-1,1-dioxidobenzo[d]isothiazol-2 (3H)-yl) pyrrolidin-2-one

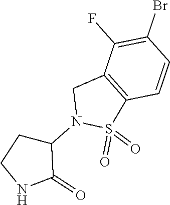 [SMILES: O=C1NCCC1N1Cc2c(ccc(Br)c2F)S1(=O)=O]

To a mixture of 5-bromo-4-fluoro-2,3-dihydrobenzo[d]isothiazole 1,1-dioxide (200 mg, 0.75 mmol) and 3-hydroxypyrrolidin-2-one (152.5 mg, 1.51 mmol) in tol (10 mL) was added Cynomethylenetributyl phosphorane (546 mg, 2.26 mmol). The mixture was stirred at 100° C. for 16 h under a nitrogen atmosphere. LCMS showed the reaction was complete. The mixture was concentrated under vacuum. The residue was purified by silica gel column chromatography, eluting with EA/PE (0-75%) to afford the product (180 mg, 68.7%). LC-MS (ESI): m/z [M+H]⁺=349.0.

### Step 2:3-(5-bromo-4-fluoro-1,1-dioxidobenzo[d]isothiazol-2 (3H)-yl)-1-methylpyrrolidin-2-one

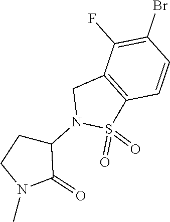 [SMILES: CN1CCC(N2Cc3c(ccc(Br)c3F)S2(=O)=O)C1=O]

To a mixture of NaH (15 mg, 0.37 mmol) in THF was added 3-(5-bromo-4-fluoro-1,1-dioxidobenzo[d]isothiazol-2 (3H)-yl) pyrrolidin-2-one (100 mg, 0.28 mmol) at 0° C. under a nitrogen atmosphere. The mixture was stirred at 0° C. for 30 mins, followed by addition of CH₃I (49 mg, 0.34 mmol). The resulting mixture was stirred at rt for 2 h. LCMS showed the reaction was complete. The mixture was quenched by ice water (50 mL), extracted with EA (3×30 mL). The combined organic layers were dried over Na₂SO₄, filtered and concentrated under vacuum. The residue was purified by silica gel column chromatography, eluting with EA/PE (0-40%) to afford the product (55 mg, 52.9%). LC-MS (ESI): m/z [M+H]⁺=363.1.

### Step 3: cis-3-(3-((4-fluoro-2-(1-methyl-2-oxopyrrolidin-3-yl)-1,1-dioxido-2,3-dihydrobenzo[d]isothiazol-5-yl)amino)-1H-pyrazol-5-yl)cyclopentyl isopropylcarbamate

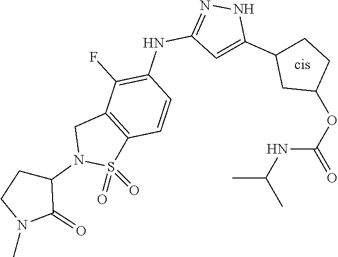 [SMILES: CC(C)NC(=O)OC1CCC(c2cc(Nc3ccc4c(c3F)CN(C3CCN(C)C3=O)S4(=O)=O)n[nH]2)C1]

A mixture of 3-(5-bromo-4-fluoro-1,1-dioxidobenzo[d]isothiazol-2 (3H)-yl)-1-methylpyrrolidin-2-one (55 mg, 0.15 mmol), cis-3-(3-amino-1H-pyrazol-5-yl)cyclopentyl isopropylcarbamate (31.8 mg, 0.13 mmol), Brettphos Pd G3 (11.5 mg, 0.012 mmol) and K₂CO₃ (53 mg, 0.378 mmol) in t-BuOH (5 mL) was stirred at 110° C. for 16 h under a nitrogen atmosphere. LCMS showed the reaction was complete. The mixture was filtered and the filtrate was concentrated under vacuum. The residue was purified by Prep-HPLC to afford the product (37.59 mg, 46.4%). ¹H NMR (500 MHz, DMSO-d₆) δ 12.00 (s, 1H), 8.80 (s, 1H), 8.35-8.28 (m, 1H), 7.55 (d, J=10 Hz, 1H), 6.94 (d, J=5 Hz, 1H), 5.83 (s, 1H), 5.04-4.96 (m, 1H), 4.69-4.63 (m, 1H), 4.47 (t, J=10 Hz, 1H), 4.34-4.28 (m, 1H), 3.62-3.52 (m, 1H), 3.39-3.33 (m, 2H), 3.11-3.01 (m, 1H), 2.80 (s, 3H), 2.48-2.43 (m, 1H), 2.38-2.29 (m, 1H), 2.28-2.19 (m, 1H), 2.08-1.98 (m, 1H), 1.96-1.85 (m, 1H), 1.77-1.66 (m, 2H), 1.64-1.55 (m, 1H), 1.07-0.97 (m, 6H). LC-MS (ESI): m/z [M+H]⁺=535.2.

### Example 94: cis-3-(3-((2-(1-benzhydrylazetidin-3-yl)-4-fluoro-1,1-dioxido-2,3-dihydrobenzo[d]isothiazol-5-yl)amino)-1H-pyrazol-5-yl)cyclopentyl isopropylcarbamate

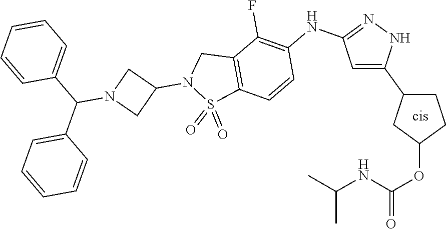 [SMILES: CC(C)NC(=O)OC1CCC(c2cc(Nc3ccc4c(c3F)CN(C3CN(C(c5ccccc5)c5ccccc5)C3)S4(=O)=O)n[nH]2)C1]

The titled compound was synthesized in the procedures similar to Example 63. ¹H NMR (500 MHz, DMSO-d₆) δ=12.01 (s, 1H), 8.81 (s, 1H), 8.29 (s, 1H), 7.52 (d, J=8.6, 1H), 7.46 (d, J=7.4, 4H), 7.30 (t, J=7.6, 4H), 7.20 (t, J=7.3, 2H), 6.94 (d, J=7.4, 1H), 5.84 (s, 1H), 5.00 (s, 1H), 4.64 (s, 2H), 4.55 (s, 1H), 4.17 (m, 1H), 3.58 (m, 1H), 3.40 (m, 2H), 3.30 (m, 2H), 3.11-2.98 (m, 1H), 2.48-2.43 (m, 1H), 2.12-1.98 (m, 1H), 1.98-1.84 (m, 1H), 1.78-1.64 (m, 2H), 1.60 (m, 1H), 1.03 (m, 6H). LC-MS (ESI): m/z [M+H]⁺=659.4.

### Example 95: cis-3-(3-((2-(cyclopropylmethyl)-4-fluoro-1,1-dioxido-2,3-dihydrobenzo[d]isothiazol-5-yl)amino)-1H-pyrazol-5-yl)cyclopentyl isopropylcarbamate

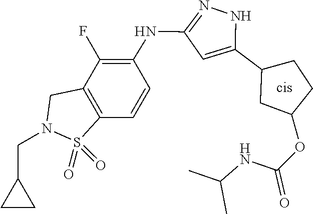 [SMILES: CC(C)NC(=O)OC1CCC(c2cc(Nc3ccc4c(c3F)CN(CC3CC3)S4(=O)=O)n[nH]2)C1]

The titled compound was synthesized in the procedures similar to Example 63. ¹H NMR (500 MHz, MeOD) δ 7.92 (t, J=7.8 Hz, 1H), 7.41 (d, J=8.6 Hz, 1H), 5.91 (s, 1H), 5.09 (s, 1H), 4.85 (s, 5H), 4.53 (s, 2H), 3.76-3.60 (m, 1H), 3.21-3.15 (m, 1H), 3.11 (d, J=7.0 Hz, 2H), 2.62-2.48 (m, 1H), 2.13 (d, J=8.1 Hz, 1H), 2.03-1.72 (m, 4H), 1.19-1.01 (m, 7H), 0.70-0.59 (m, 2H), 0.35 (q, J=5.0 Hz, 2H). LC-MS (ESI): m/z [M+H]⁺=492.2.

### Example 96: cis-3-(3-((2-(cyclobutylmethyl)-4-fluoro-1,1-dioxido-2,3-dihydrobenzo[d]isothiazol-5-yl)amino)-1H-pyrazol-5-yl)cyclopentyl isopropylcarbamate

### Step 1:5-bromo-2-(cyclobutylmethyl)-4-fluoro-2,3-dihydrobenzo[d]isothiazole 1,1-dioxide

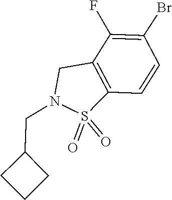 [SMILES: O=S1(=O)c2ccc(Br)c(F)c2CN1CC1CCC1]

To a stirring solution of 5-bromo-4-fluoro-2,3-dihydrobenzo[d]isothiazole 1,1-dioxide (220.5 mg, 0.75 mmol) in DMF (10 mL) was added Cs₂CO₃ (489.5 mg, 1.50 mmol), (bromomethyl)cyclobutane (820.7 mg, 1.50 mmol). The solution was stirred for 3 h at 50° C. The mixture was concentrated under reduced pressure. The residue was purified by silica gel column chromatography, eluting with PE/EtOAc (3:1) to afford the product (235 mg, 75%), [M+H]⁺=333.9.

### Step 2:3-(3-((2-(cyclobutylmethyl)-4-fluoro-1,1-dioxido-2,3-dihydrobenzo[d]isothiazol-5-yl)amino)-1H-pyrazol-5-yl)cyclopentyl isopropylcarbamate

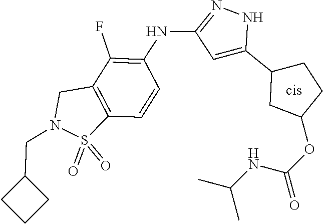 [SMILES: CC(C)NC(=O)OC1CCC(c2cc(Nc3ccc4c(c3F)CN(CC3CCC3)S4(=O)=O)n[nH]2)C1]

A mixture of 5-bromo-2-(cyclobutylmethyl)-4-fluoro-2,3-dihydrobenzo[d]isothiazole 1,1-dioxide (50 mg, 0.15 mmol), cis-3-(3-amino-1H-pyrazol-5-yl)cyclopentyl isopropylcarbamate (37.8 mg, 0.15 mmol), Brettphos Pd G3 (13.5 mg, 0.015 mmol), K₂CO₃ (62.1 mg, 0.45 mmol) in t-BuOH (10 mL) was stirred at 110° C. for 16 h under N₂ atmosphere. The mixture was concentrated under reduced pressure. The residue was purified by silica gel column chromatography. The residue was purified by prep HPLC (Waters XSelect C18: RD-CO-094 column, eluting with a gradient of acetonitrile/water containing 0.1% FA, 20%-40%) to afford the product (72.6 mg, 96.1%). ¹H NMR (500 MHz, DMSO-d₆) δ 11.70 (s, 1H), 8.04 (s, 1H), 7.91 (s, 1H), 6.88 (d, J=8.2 Hz, 1H), 6.68 (d, J=8.7 Hz, 1H), 5.68 (s, 1H), 4.99 (s, 1H), 4.72 (s, 2H), 3.44-3.34 (m, 1H), 3.08-2.94 (m, 1H), 2.50-2.44 (m, 1H), 2.06-1.94 (m, 1H), 1.95-1.85 (m, 1H), 1.77-1.65 (m, 2H), 1.65-1.55 (m, 1H), 1.40-1.30 (m, 2H), 1.06-0.94 (m, 3H), 0.84-0.78 (m, 3H). LC-MS (ESI): m/z [M+H]⁺=506.2.

### Example 97: cis-3-(3-((4-fluoro-2-(oxetan-3-ylmethyl)-1,1-dioxido-2,3-dihydrobenzo[d]isothiazol-5-yl)amino)-1H-pyrazol-5-yl)cyclopentyl isopropylcarbamate

### Step 1:5-bromo-4-fluoro-2-(oxetan-3-ylmethyl)-2,3-dihydrobenzo[d]isothiazole 1,1-dioxide

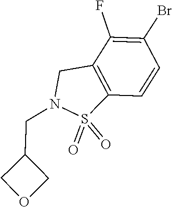 [SMILES: O=S1(=O)c2ccc(Br)c(F)c2CN1CC1COC1]

To a stirring solution of 5-bromo-4-fluoro-2,3-dihydrobenzo[d]isothiazole 1,1-dioxide (250 mg, 0.94 mmol) in Toluene (20 mL) was added oxetan-3-ylmethanol (166.03 mg, 1.89 mmol) CMBP (688.1 mg, 2.82 mmol). The solution was stirred for 90° C. for 16 h under N₂ atmosphere. The mixture was concentrated under reduced pressure. The residue was purified by silica gel column chromatography, eluting with PE/EtOAc (3:1) to afford the product (380 mg, 41.8%), [M+H]⁺=335.9.

### Step 2: cis-3-(3-((4-fluoro-2-(oxetan-3-ylmethyl)-1,1-dioxido-2,3-dihydrobenzo[d]isothiazol-5-yl)amino)-1H-pyrazol-5-yl)cyclopentyl isopropylcarbamate

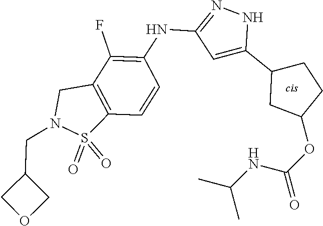 [SMILES: CC(C)NC(=O)OC1CCC(c2cc(Nc3ccc4c(c3F)CN(CC3COC3)S4(=O)=O)n[nH]2)C1]

A mixture of 5-bromo-4-fluoro-2-(oxetan-3-ylmethyl)-2,3-dihydrobenzo[d]isothiazole 1,1-dioxide (80 mg, 0.24 mmol), cis-3-(3-amino-1H-pyrazol-5-yl)cyclopentyl isopropylcarbamate (60.2 mg, 0.24 mmol), Brettphos Pd G3 (21.6 mg, 0.024 mmol), K₂CO₃ (99.3 mg, 0.72 mmol) in t-BuOH (20 mL) was stirred at 110° C. for 16 h under N₂ atmosphere. The mixture was concentrated under reduced pressure. The residue was purified by silica gel column chromatography, eluting with EA to afford the product 80 mg. The residue was purified by prep HPLC (Waters XSelect C18: RD-CO-094 column, eluting with a gradient of acetonitrile/water containing 0.1% FA, 20%-40%) to afford the product (79.1 mg). ¹H NMR (500 MHz, DMSO-d₆) δ 12.00 (s, 1H), 8.78 (s, 1H), 8.30 (s, 1H), 7.54 (d, J=8.6 Hz, 1H), 6.94 (d, J=7.4 Hz, 1H), 5.83 (s, 1H), 5.00 (s, 1H), 4.69 (dd, J=7.5, 6.3 Hz, 2H), 4.46 (s, 2H), 4.36 (t, J=6.0 Hz, 2H), 3.62-3.56 (m, 1H), 3.47 (d, J=7.5 Hz, 2H), 3.40-3.36 (m, 1H), 3.15-2.97 (m, 1H), 2.47-2.40 (m, 1H), 2.06-1.99 (m, 1H), 1.96-1.86 (m, 1H), 1.78-1.66 (m, 2H), 1.65-1.57 (m, 1H), 1.10-0.93 (m, 6H). LC-MS (ESI): m/z [M+H]⁺=508.3.

### Example 98: cis-3-(3-((2-((S)-2-amino-3,3-dimethylbutyl)-4-fluoro-1,1-dioxido-2,3-dihydrobenzo[d]isothiazol-5-yl)amino)-1H-pyrazol-5-yl)cyclopentyl isopropylcarbamate

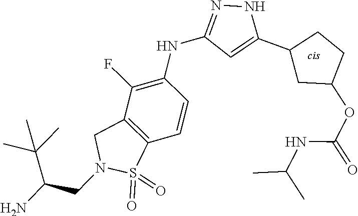 [SMILES: CC(C)NC(=O)OC1CCC(c2cc(Nc3ccc4c(c3F)CN(C[C@@H](N)C(C)(C)C)S4(=O)=O)n[nH]2)C1]

The titled compound was synthesized in the procedures similar to Example 63. ¹H NMR (500 MHz, DMSO-d₆) δ 12.00 (s, 1H), 8.79 (s, 1H), 8.29 (s, 1H), 7.56 (d, J=8.6 Hz, 1H), 6.94 (d, J=6.9 Hz, 1H), 5.83 (s, 1H), 5.00 (s, 1H), 4.61 (d, J=14.2 Hz, 1H), 4.49 (d, J=14.1 Hz, 1H), 3.60-3.58 (m, 1H), 3.26 (d, J=13.4 Hz, 1H), 3.10-3.02 (m, 1H), 2.94-2.85 (m, 1H), 2.77 (d, J=10.3 Hz, 1H), 2.50-2.46 (m, 1H), 2.06-1.94 (m, 1H), 1.95-1.85 (m, 1H), 1.77-1.65 (m, 2H), 1.65-1.55 (m, 1H), 1.03 (d, J=5.9 Hz, 6H), 0.92 (s, 9H). LC-MS (ESI): m/z [M+H]⁺=537.2.

### Example 99: cis-3-(3-((2-((R)-2-amino-3,3-dimethylbutyl)-4-fluoro-1,1-dioxido-2,3-dihydrobenzo[d]isothiazol-5-yl)amino)-1H-pyrazol-5-yl)cyclopentyl isopropylcarbamate

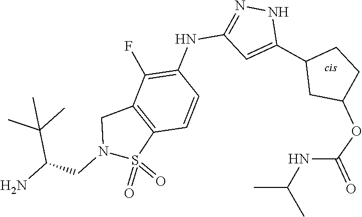 [SMILES: CC(C)NC(=O)OC1CCC(c2cc(Nc3ccc4c(c3F)CN(C[C@H](N)C(C)(C)C)S4(=O)=O)n[nH]2)C1]

The titled compound was synthesized in the procedures similar to Example 63. ¹H NMR (500 MHz, DMSO-d₆) δ 12.00 (s, 1H), 8.79 (s, 1H), 8.29 (s, 1H), 7.56 (d, J=8.6 Hz, 1H), 6.94 (d, J=6.9 Hz, 1H), 5.83 (s, 1H), 5.00 (s, 1H), 4.61 (d, J=14.2 Hz, 1H), 4.49 (d, J=14.1 Hz, 1H), 3.60-3.58 (m, 1H), 3.26 (d, J=13.4 Hz, 1H), 3.10-3.02 (m, 1H), 2.94-2.85 (m, 1H), 2.77 (d, J=10.3 Hz, 1H), 2.50-2.46 (m, 1H), 2.06-1.94 (m, 1H), 1.95-1.85 (m, 1H), 1.77-1.65 (m, 2H), 1.65-1.55 (m, 1H), 1.03 (d, J=5.9 Hz, 6H), 0.92 (s, 9H). LC-MS (ESI): m/z [M+H]⁺=537.2.

### Example 100: cis-3-(3-((4-fluoro-2-(1-methyl-2-oxopiperidin-4-yl)-1,1-dioxido-2,3-dihydrobenzo[d]isothiazol-5-yl)amino)-1H-pyrazol-5-yl)cyclopentyl isopropylcarbamate

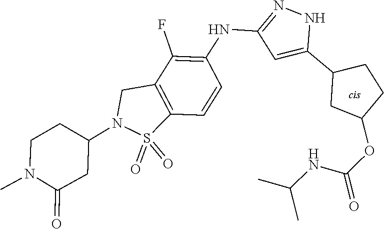 [SMILES: CC(C)NC(=O)OC1CCC(c2cc(Nc3ccc4c(c3F)CN(C3CCN(C)C(=O)C3)S4(=O)=O)n[nH]2)C1]

The titled compound was synthesized in the procedures similar to Example 63. LC-MS (ESI): m/z [M+H]⁺=549.2.

### Example 101: cis-3-(3-((4-fluoro-2-isobutyl-1,1-dioxido-2,3-dihydrobenzo[d]isothiazol-5-yl)amino)-1H-pyrazol-5-yl)cyclopentyl isopropylcarbamate

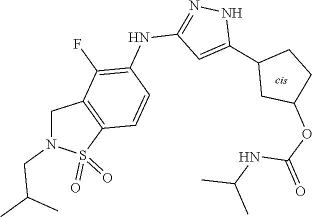 [SMILES: CC(C)CN1Cc2c(ccc(Nc3cc(C4CCC(OC(=O)NC(C)C)C4)[nH]n3)c2F)S1(=O)=O]

The titled compound was synthesized in the procedures similar to Example 35. ¹H NMR (500 MHz, DMSO-d₆) δ 11.99 (s, 1H), 8.77 (s, 1H), 8.30 (t, J=7.6 Hz, 1H), 7.54 (d, J=8.6 Hz, 1H), 6.94 (d, J=7.4 Hz, 1H), 5.83 (s, 1H), 4.99 (s, 1H), 4.47 (s, 2H), 3.58 (d, J=6.7 Hz, 1H), 3.13-3.03 (m, 1H), 2.92 (d, J=7.3 Hz, 2H), 2.44-2.38 (m, 1H), 2.10-1.95 (m, 2H), 1.93-1.84 (m, 1H), 1.75-1.65 (m, 2H), 1.59-1.52 (m, 1H), 1.03 (d, J=6.2 Hz, 6H), 0.95 (d, J=6.6 Hz, 7H). LC-MS (ESI): m/z [M+H]⁺=494.2.

### Example 102 and 103: cis-3-(3-((4-fluoro-2-(2-hydroxycyclobutyl)-1,1-dioxido-2,3-dihydrobenzo[d]isothiazol-5-yl)amino)-1H-pyrazol-5-yl)cyclopentyl isopropylcarbamate

### Step 1:2-(5-bromo-4-fluoro-1,1-dioxidobenzo[d]isothiazol-2 (3H)-yl)cyclobutan-1-one

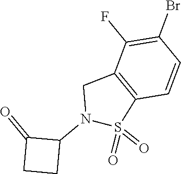 [SMILES: O=C1CCC1N1Cc2c(ccc(Br)c2F)S1(=O)=O]

A mixture of 2-bromocyclobutan-1-one (0.252 g, 1.69 mmol), K₂CO₃ (0.233 g, 1.69 mmol) and 5-bromo-4-fluoro-2,3-dihydrobenzo[d]isothiazole 1,1-dioxide (0.3 g, 1.12 mmol) in DMF (10 mL) was stirred at 50° C. for 12 hr. The mixture was quenched with water (30 mL). The mixture was extracted by DCM (20 mL×3). The DCM layer was concentrated under reduced pressure. The residue was purified by silica gel column chromatography, eluting with PE/EtOAc (5:1) to afford the product (0.3 g, 79.8%). LC-MS (ESI): m/z [M+H]⁺=334.2.

### Step 2:5-bromo-4-fluoro-2-(2-hydroxycyclobutyl)-2,3-dihydrobenzo[d]isothiazole 1,1-dioxide

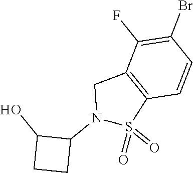 [SMILES: O=S1(=O)c2ccc(Br)c(F)c2CN1C1CCC1O]

To a mixture of 2-(5-bromo-4-fluoro-1,1-dioxidobenzo[d]isothiazol-2 (3H)-yl)cyclobutan-1-one (0.3 g, 0.9 mmol) in EtOH (20 mL), NaBH₄ (0.055 g, 1.45 mmol) was added. The mixture was stirred at 25° C. for 2 hrs. The mixture was quenched with saturated NH₄Cl (aq.) (10 mL) and extracted by DCM (30 mL×3). The DCM layer was concentrated under reduced pressure to afford the crude product, which was used for next step without purification. (0.26 g, 86.1%). LC-MS (ESI): m/z [M+H]⁺=336.2.

### Step 3: cis-3-(3-((4-fluoro-2-(2-hydroxycyclobutyl)-1,1-dioxido-2,3-dihydrobenzo[d]isothiazol-5-yl)amino)-1H-pyrazol-5-yl)cyclopentyl isopropylcarbamate

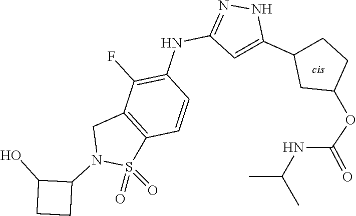 [SMILES: CC(C)NC(=O)OC1CCC(c2cc(Nc3ccc4c(c3F)CN(C3CCC3O)S4(=O)=O)n[nH]2)C1]

The titled compound was synthesized in the procedures similar to Example 34, step 5.

Diastereoisomer 1 (Example 102): ¹H NMR (500 MHz, DMSO-d₆) δ 11.98 (s, 1H), 8.76 (s, 1H), 8.28 (s, 1H), 7.51 (d, J=8.6 Hz, 1H), 6.92 (s, 1H), 5.83 (s, 1H), 5.50 (d, J=7.4 Hz, 1H), 5.00 (s, 1H), 4.60 (d, J=14.2 Hz, 1H), 4.39 (d, J=14.2 Hz, 1H), 4.23-4.11 (m, 1H), 3.61 (dt, J=13.0, 7.5 Hz, 2H), 3.15-3.04 (m, 1H), 2.48-2.43 (m, 1H), 2.14-1.99 (m, 2H), 1.95-1.85 (m, 2H), 1.78-1.65 (m, 3H), 1.65-1.52 (m, 2H), 1.03 (d, J=5.8 Hz, 6H). LC-MS (ESI): m/z [M+H]⁺=508.3.

Diastereoisomer 2 (Example 103): ¹H NMR (500 MHz, DMSO-d₆) δ 11.98 (s, 1H), 8.76 (s, 1H), 8.29 (s, 1H), 7.52 (d, J=8.6 Hz, 1H), 6.92 (s, 1H), 5.83 (s, 1H), 5.39 (d, J=5.3 Hz, 1H), 5.00 (s, 1H), 4.79 (d, J=14.5 Hz, 1H), 4.59 (d, J=14.5 Hz, 1H), 4.42 (s, 1H), 4.0-3.93 (m, 1H), 3.62-3.53 (s, 1H), 3.11-3.02 (m, 1H), 2.48-2.43 (m, 1H), 2.14-1.99 (m, 2H), 1.95-1.85 (m, 2H), 1.78-1.55 (m, 3H), 1.65-1.52 (m, 2H), 1.03 (d, J=5.8 Hz, 6H). LC-MS (ESI): m/z [M+H]⁺=508.3.

### Example 104: cis-3-(3-((4-fluoro-2-(1-(oxetan-3-yl)ethyl)-1,1-dioxido-2,3-dihydrobenzo[d]isothiazol-5-yl)amino)-1H-pyrazol-5-yl)cyclopentyl isopropylcarbamate

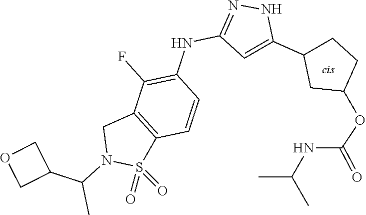 [SMILES: CC(C)NC(=O)OC1CCC(c2cc(Nc3ccc4c(c3F)CN(C(C)C3COC3)S4(=O)=O)n[nH]2)C1]

The titled compound was synthesized in the procedures similar to Example 63. ¹H NMR (500 MHz, DMSO-d₆) δ 11.99 (s, 1H), 8.76 (s, 1H), 8.29 (s, 1H), 7.52 (d, J=8.6 Hz, 1H), 6.93 (d, J=6.5 Hz, 1H), 5.83 (s, 1H), 4.99 (s, 1H), 4.66 (t, J=7.0 Hz, 1H), 4.59 (t, J=6.9 Hz, 1H), 4.49 (d, J=14.1 Hz, 1H), 4.41-4.25 (m, 4H), 4.12-4.03 (m, 1H), 3.62-3.53 (m, 1H), 3.29-3.23 (m, 1H), 3.12-3.02 (m, 1H), 2.48-2.43 (m, 1H), 2.07-1.86 (m, 2H), 1.77-1.55 (m, 3H), 1.20 (d, J=6.5 Hz, 3H), 1.03 (d, J=5.8 Hz, 6H). LC-MS (ESI): m/z [M+H]⁺=522.3.

### Example 105: cis-3-(3-((2-(azetidin-3-ylmethyl)-4-fluoro-1,1-dioxido-2,3-dihydrobenzo[d]isothiazol-5-yl)amino)-1H-pyrazol-5-yl)cyclopentyl isopropylcarbamate

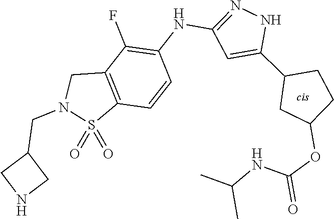 [SMILES: CC(C)NC(=O)OC1CCC(c2cc(Nc3ccc4c(c3F)CN(CC3CNC3)S4(=O)=O)n[nH]2)C1]

The titled compound was synthesized in the procedures similar to Example 63. ¹H NMR (500 MHz, DMSO-d₆) δ 12.02 (s, 1H), 8.81 (s, 1H), 8.65 (s, 1H), 8.54 (s, 1H), 8.31 (s, 1H), 7.57 (d, J=8.7 Hz, 1H), 6.93 (d, J=6.9 Hz, 1H), 5.84 (s, 1H), 5.00 (s, 1H), 4.51 (s, 2H), 3.85-3.75 (m, 2H), 3.65-3.55 (m, 1H), 3.46 (d, J=7.4 Hz, 2H), 3.25-3.15 (m, 1H), 3.11-2.98 (m, 1H), 2.48-2.40 (m, 1H), 2.10-1.98 (m, 1H), 1.96-1.85 (m, 1H), 1.84-1.72 (m, 1H), 1.75-1.65 (m, 2H), 1.58-1.52 (m, 1H), 1.03 (d, J=5.6 Hz, 6H). LC-MS (ESI): m/z [M+H]⁺=507.2.

### Example 106: cis-3-(3-((4-fluoro-2-((1-methylazetidin-3-yl)methyl)-1,1-dioxido-2,3-dihydrobenzo[d]isothiazol-5-yl)amino)-1H-pyrazol-5-yl)cyclopentyl isopropylcarbamate

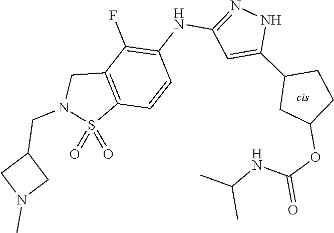 [SMILES: CC(C)NC(=O)OC1CCC(c2cc(Nc3ccc4c(c3F)CN(CC3CN(C)C3)S4(=O)=O)n[nH]2)C1]

The titled compound was synthesized in the procedures similar to Example 63. ¹H NMR (500 MHz, DMSO-d₆) δ 12.05 (s, 1H), 8.78 (s, 1H), 8.28 (t, J=7.8 Hz, 1H), 7.54 (d, J=8.6 Hz, 1H), 6.94 (d, J=7.1 Hz, 1H), 5.83 (s, 1H), 5.00 (s, 1H), 4.46 (s, 2H), 3.65-3.54 (m, 1H), 3.48 (t, J=7.6 Hz, 2H), 3.36 (d, J=7.4 Hz, 2H), 3.15 (t, J=6.7 Hz, 2H), 3.10-3.00 (m, 1H), 2.81-2.78 (m, 1H), 2.48-2.40 (m, 1H), 2.35 (s, 3H), 2.10-1.99 (m, 1H), 1.96-1.84 (m, 1H), 1.81-1.67 (m, 2H), 1.58-1.52 (m, 1H), 1.03 (d, J=5.7 Hz, 6H). LC-MS (ESI): m/z [M+H]⁺=521.2.

### Example 107: cis-3-(3-((2-((1-acetylazetidin-3-yl)methyl)-4-fluoro-1,1-dioxido-2,3-dihydrobenzo[d]isothiazol-5-yl)amino)-1H-pyrazol-5-yl)cyclopentyl isopropylcarbamate

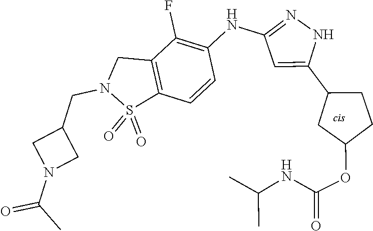 [SMILES: CC(=O)N1CC(CN2Cc3c(ccc(Nc4cc(C5CCC(OC(=O)NC(C)C)C5)[nH]n4)c3F)S2(=O)=O)C1]

The titled compound was synthesized in the procedures similar to Example 63. ¹H NMR (500 MHz, DMSO-d₆) δ 12.00 (s, 1H), 8.80 (s, 1H), 8.31 (t, J=7.7 Hz, 1H), 7.55 (d, J=8.7 Hz, 1H), 6.94 (d, J=6.5 Hz, 1H), 5.83 (s, 1H), 5.00 (s, 1H), 4.50 (s, 2H), 4.21 (t, J=8.3 Hz, 1H), 3.98-3.81 (m, 2H), 3.66-3.50 (m, 2H), 3.43-3.37 (m, 2H), 3.12-3.04 (m, 1H), 3.02-2.90 (m, 1H), 2.50-2.44 (m, 1H), 2.07-1.97 (m, 1H), 1.96-1.85 (m, 1H), 1.74 (s, 3H), 1.75-1.65 (m, 2H), 1.58-1.52 (m, 1H), 1.03 (d, J=3.6 Hz, 6H). LC-MS (ESI): m/z [M+H]⁺=549.2.

### Example 108: cis-3-(3-((2-(cyclohexylmethyl)-4-fluoro-1,1-dioxido-2,3-dihydrobenzo[d]isothiazol-5-yl)amino)-1H-pyrazol-5-yl)cyclopentyl isopropylcarbamate

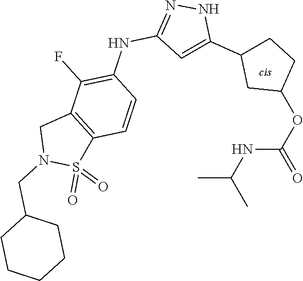 [SMILES: CC(C)NC(=O)OC1CCC(c2cc(Nc3ccc4c(c3F)CN(CC3CCCCC3)S4(=O)=O)n[nH]2)C1]

dihydrobenzo[d]isothiazol-5-yl)amino)-1H-pyrazol-5-yl)cyclopentyl isopropylcarbamate

To a mixture of 3-(3-((4-fluoro-1,1-dioxido-2,3-dihydrobenzo[d]isothiazol-5-yl)amino)-1H-pyrazol-5-yl)cyclopentyl isopropylcarbamate (60 mg, 0.14 mmol) and cyclohexylmethanol (23.4 mg, 0.01 mmol) in tol was added cynomethylenetributyl phosphorane (84 mg, 0.34 mmol). The mixture was stirred 95° C. for 16 h under a nitrogen atmosphere. The mixture was concentrated under vacuum. The residue was purified by Prep-HPLC to afford the product (6.8 mg, 9.3%). ¹H NMR (500 MHz, DMSO-d₆) δ 11.99 (s, 1H), 8.77 (s, 1H), 8.34-8.25 (m, 1H), 7.53 (d, J=10 Hz, 1H), 6.94 (d, J=10 Hz, 1H), 5.83 (s, 1H), 5.03-4.96 (m, 1H), 4.47 (s, 3H), 3.62-3.53 (m, 1H), 3.11-3.03 (m, 1H), 2.98-2.92 (m, 2H), 2.48-2.43 (m, 1H), 2.06-1.98 (m, 1H), 1.95-1.86 (m, 1H), 1.83-1.51 (m, 10H), 1.30-1.12 (m, 1H), 1.07-0.98 (m, 6H), 0.97-0.89 (m, 2H). LC-MS (ESI): m/z [M+H]⁺=534.2.

### Example 109: cis-3-(3-((4-fluoro-2-(2-hydroxycyclopentyl)-1,1-dioxido-2,3-dihydrobenzo[d]isothiazol-5-yl)amino)-1H-pyrazol-5-yl)cyclopentyl isopropylcarbamate

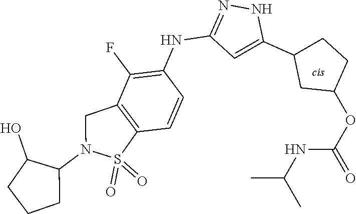 [SMILES: CC(C)NC(=O)OC1CCC(c2cc(Nc3ccc4c(c3F)CN(C3CCCC3O)S4(=O)=O)n[nH]2)C1]

The titled compound was synthesized in the procedures similar to Example 63. ¹H NMR (500 MHz, DMSO-d₆) δ=11.99 (s, 1H), 8.76 (s, 1H), 8.29 (s, 1H), 7.52 (d, J=8.6, 1H), 6.94 (d, J=7.1, 1H), 5.83 (s, 1H), 4.99 (s, 1H), 4.73 (d, J=4.1, 1H), 4.66 (d, J=14.3, 1H), 4.58 (d, J=14.3, 1H), 4.22 (m, 1H), 3.58 (m, 1H), 3.47 (m, 1H), 3.13-2.97 (m, 1H), 2.48-2.43 (m, 1H), 2.03 (m, 2H), 1.90 (m, 2H), 1.85-1.76 (m, 2H), 1.76-1.65 (m, 2H), 1.58 (m, 3H), 1.03 (m, 6H). LC-MS (ESI): m/z [M+H]⁺=522.32.

### Example 110: cis-3-(3-((4-fluoro-1,1-dioxido-2-((2-oxopyrrolidin-3-yl)methyl)-2,3-dihydrobenzo[d]isothiazol-5-yl)amino)-1H-pyrazol-5-yl)cyclopentyl isopropylcarbamate

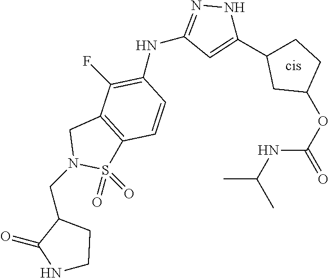 [SMILES: CC(C)NC(=O)OC1CCC(c2cc(Nc3ccc4c(c3F)CN(CC3CCNC3=O)S4(=O)=O)n[nH]2)C1]

The titled compound was synthesized in the procedures similar to Example 63. ¹H NMR (500 MHz, DMSO-d₆) δ 12.00 (s, 1H), 8.79 (s, 1H), 8.31 (t, J=10 Hz, 1H), 7.78 (s, 1H), 7.55 (d, J=10 Hz, 1H), 6.94 (d, J=5 Hz, 1H), 5.83 (s, 1H), 5.05-4.95 (m, 1H), 4.56-4.44 (m, 2H), 3.64-3.53 (m, 1H), 3.52-3.46 (m, 1H), 3.26-3.13 (m, 3H), 3.12-3.01 (m, 1H), 2.73-2.62 (m, 1H), 2.47-2.44 (m, 1H), 2.31-2.22 (m, 1H), 2.06-1.87 (m, 3H), 1.77-1.67 (m, 2H), 1.64-1.55 (m, 1H), 1.10-1.95 (m, 6H). LC-MS (ESI): m/z [M+H]⁺=535.2.

### Example 111: cis-3-(3-((4-fluoro-2-((1-methyl-2-oxopyrrolidin-3-yl)methyl)-1,1-dioxido-2,3-dihydrobenzo[d]isothiazol-5-yl)amino)-1H-pyrazol-5-yl)cyclopentyl isopropylcarbamate

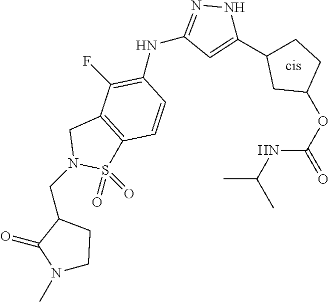 [SMILES: CC(C)NC(=O)OC1CCC(c2cc(Nc3ccc4c(c3F)CN(CC3CCN(C)C3=O)S4(=O)=O)n[nH]2)C1]

The titled compound was synthesized in the procedures similar to Example 63. ¹H NMR (500 MHz, DMSO-d₆) δ 12.00 (s, 1H), 8.79 (s, 1H), 8.34-8.25 (m, 1H), 7.55 (t, J=10 Hz, 1H), 6.94 (t, J=10 Hz, 1H), 5.83 (s, 1H), 5.04-4.96 (m, 1H), 4.52-4.46 (m, 2H), 3.62-3.47 (m, 2H), 3.30-3.26 (m, 2H), 3.23-3.16 (m, 1H), 3.11-3.02 (m, 1H), 2.80-2.72 (m, 4H), 2.48-2.43 (m, 1H), 2.25-2.17 (m, 1H), 2.07-1.99 (m, 1H), 1.96-1.84 (m, 2H), 1.77-1.65 (m, 2H), 1.64-1.55 (m, 1H), 1.08-0.96 (m, 6H). LC-MS (ESI): m/z [M+H]⁺=549.2.

### Example 112: cis-3-(3-((4-fluoro-2-(2-morpholinoethyl)-1,1-dioxido-2,3-dihydrobenzo[d]isothiazol-5-yl)amino)-1H-pyrazol-5-yl)cyclopentyl isopropylcarbamate

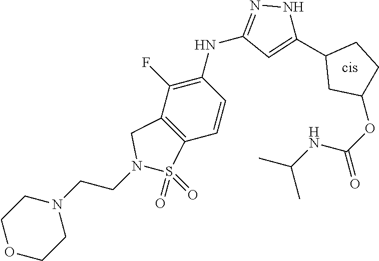 [SMILES: CC(C)NC(=O)OC1CCC(c2cc(Nc3ccc4c(c3F)CN(CCN3CCOCC3)S4(=O)=O)n[nH]2)C1]

The titled compound was synthesized in the procedures similar to Example 63. ¹H NMR (500 MHz, DMSO-d₆) δ=12.00 (s, 1H), 8.77 (s, 1H), 8.29 (t, J=7.8, 1H), 7.54 (d, J=8.6, 1H), 6.94 (d, J=7.4, 1H), 5.84 (s, 1H), 5.00 (s, 1H), 4.56 (s, 2H), 3.62-3.51 (m, 5H), 3.29 (t, J=6.6, 2H), 3.12-3.00 (m, 1H), 2.63 (t, J=6.6, 2H), 2.45 (m, 5H), 2.03 (m, 1H), 1.91 (m, 1H), 1.70 (m, 2H), 1.60 (m, 1H), 1.08-0.96 (m, 6H). LC-MS (ESI): m/z [M+H]⁺=551.36.

### Example 113: cis-3-(3-((4-fluoro-2-(3-hydroxypropyl)-1,1-dioxido-2,3-dihydrobenzo[d]isothiazol-5-yl)amino)-1H-pyrazol-5-yl)cyclopentyl isopropylcarbamate

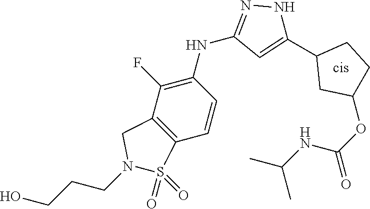 [SMILES: CC(C)NC(=O)OC1CCC(c2cc(Nc3ccc4c(c3F)CN(CCCO)S4(=O)=O)n[nH]2)C1]

The titled compound was synthesized in the procedures similar to Example 37. ¹H NMR (500 MHz, DMSO-d₆) δ 12.00 (s, 1H), 8.77 (s, 1H), 8.28 (s, 1H), 7.53 (d, J=8.6 Hz, 1H), 7.02-6.87 (m, 1H), 5.83 (s, 1H), 5.04-4.92 (m, 1H), 4.47 (s, 2H), 3.65-3.57 (m, 2H), 3.51 (t, J=6.1 Hz, 2H), 3.21 (t, J=6.1 Hz, 2H), 3.14-3.00 (m, 1H), 2.48-2.42 (m, 1H), 2.08-1.99 (m, 1H), 1.97-1.87 (m, 1H), 1.86-1.78 (m, 2H), 1.77-1.66 (m, 2H), 1.65-1.54 (m, 1H), 1.09-0.98 (m, 6H). LC-MS (ESI): m/z [M+H]⁺=496.29.

### Example 114: cis-3-(3-((4-fluoro-1,1-dioxido-2-(2-(tetrahydro-2H-pyran-4-yl)ethyl)-2,3-dihydrobenzo[d]isothiazol-5-yl)amino)-1H-pyrazol-5-yl)cyclopentyl isopropylcarbamate

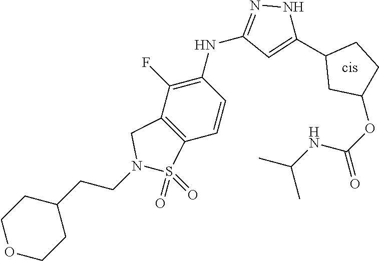 [SMILES: CC(C)NC(=O)OC1CCC(c2cc(Nc3ccc4c(c3F)CN(CCC3CCOCC3)S4(=O)=O)n[nH]2)C1]

The titled compound was synthesized in the procedures similar to Example 63. ¹H NMR (500 MHz, DMSO-d₆) δ 12.00 (s, 1H), 8.78 (s, 1H), 8.30 (t, J=8.0 Hz, 1H), 7.53 (d, J=8.7 Hz, 1H), 6.94 (d, J=7.0 Hz, 1H), 5.83 (s, 1H), 5.00 (s, 1H), 4.47 (s, 2H), 3.83 (dd, J=10.9, 3.6 Hz, 2H), 3.63-3.53 (m, 1H), 3.31-3.24 (m, 2H), 3.17 (t, J=6.8 Hz, 2H), 3.11-3.03 (m, 1H), 2.48-2.43 (m, 1H), 2.08-1.87 (m, 2H), 1.74-1.58 (m, 8H), 1.25-1.14 (m, 2H), 1.07-0.99 (m, 6H). LC-MS (ESI): m/z [M+H]⁺=550.3.

### Example 115: cis-3-(3-((4-fluoro-1,1-dioxido-2-(1-(tetrahydrofuran-3-yl)ethyl)-2,3-dihydrobenzo[d]isothiazol-5-yl)amino)-1H-pyrazol-5-yl)cyclopentyl isopropylcarbamate

### Step 1:1-(tetrahydrofuran-3-yl)ethan-1-ol

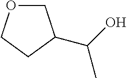

To a solution of tetrahydrofuran-3-carbaldehyde (2.0 g, 20 mmol) in THF (40 mL), MeMgBr (30 mL, 30 mmol) was added at 0° C. Then the mixture was stirred at 25° C. for 1 hr. The mixture was quenched with saturated NH₄Cl (aq.) (20 mL). The mixture was extracted by EA (30 mL×3). The EA layer was concentrated under reduced pressure. The residue was purified by silica gel column chromatography, eluting with PE/EtOAc (1:1) to afford the product (1.5 g, 64.6%). LC-MS (ESI): m/z [M+H]⁺=117.1.

### Step 2:5-bromo-4-fluoro-2-(1-(tetrahydrofuran-3-yl)ethyl)-2,3-dihydrobenzo[d]isothiazole 1,1-dioxide

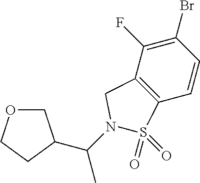 [SMILES: CC(C1CCOC1)N1Cc2c(ccc(Br)c2F)S1(=O)=O]

A mixture of 1-(tetrahydrofuran-3-yl)ethan-1-ol (0.197 g, 1.7 mmol), CMBP (0.816 g, 3.38 mmol) and 5-bromo-4-fluoro-2,3-dihydrobenzo[d]isothiazole 1,1-dioxide (0.3 g, 1.13 mmol) in toluene (10 mL) was stirred at 95° C. under N₂ for 12 hr. The mixture was concentrated under reduced pressure. The residue was purified by silica gel column chromatography, eluting with PE/EtOAc (10: 1~5:1) to afford the product (0.33 g, 80.5%). LC-MS (ESI): m/z [M+H]⁺=364.

### Step 3: cis-3-(3-((4-fluoro-1,1-dioxido-2-(1-(tetrahydrofuran-3-yl)ethyl)-2,3-dihydrobenzo[d]isothiazol-5-yl)amino)-1H-pyrazol-5-yl)cyclopentyl isopropylcarbamate

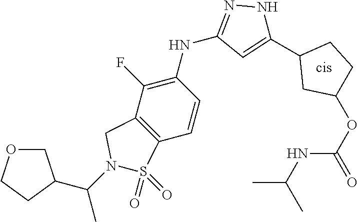 [SMILES: CC(C)NC(=O)OC1CCC(c2cc(Nc3ccc4c(c3F)CN(C(C)C3CCOC3)S4(=O)=O)n[nH]2)C1]

The titled compound was synthesized in the procedures similar to Example 34, step 5. ¹H NMR (500 MHz, DMSO-d₆) δ 11.99 (s, 1H), 8.77 (s, 1H), 8.31 (s, 1H), 7.56-7.48 (m, 1H), 6.94 (d, J=7.3 Hz, 1H), 5.83 (s, 1H), 5.00 (s, 1H), 4.57-4.45 (m, 2H), 3.89-3.72 (m, 2H), 3.67-3.51 (m, 3H), 3.47-3.43 (m, 1H), 3.11-3.02 (m, 1H), 2.48-2.43 (m, 1H), 2.11-1.80 (m, 4H), 1.76-1.58 (m, 4H), 1.26 (dd, J=34.0, 6.6 Hz, 3H), 1.03 (d, J=6.2 Hz, 6H). LC-MS (ESI): m/z [M+H]⁺=536.3.

### Example 116: cis-3-(3-((2-cyclopropyl-4-fluoro-1,1-dioxido-2,3-dihydrobenzo[d]isothiazol-5-yl)amino)-1H-pyrazol-5-yl)cyclopentyl isopropylcarbamate

### Step 1: methyl 3-bromo-6-(N-cyclopropylsulfamoyl)-2-fluorobenzoate

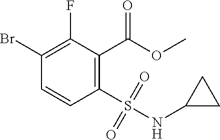 [SMILES: COC(=O)c1c(S(=O)(=O)NC2CC2)ccc(Br)c1F]

To a solution of cyclopropanamine (2.7 g, 48 mmol) and TEA (12.1 g, 120 mmol) in DCM (150 mL) was added dropwise a solution of methyl 3-bromo-6-(chlorosulfonyl)-2-fluorobenzoate (13.3 g, 40 mmol) in DCM (30 mL) at 0° C. The mixture was stirred at rt for 1 h. LCMS showed the reaction was complete. The mixture was concentrated under vacuum. The residue was purified by silica gel column chromatography, eluting with EA/PE (0-40%) to afford the product (6.6 g, 46.5%). LC-MS (ESI): m/z [M+H]⁺=352.2.

### Step 2:3-bromo-6-(N-cyclopropylsulfamoyl)-2-fluorobenzoic acid

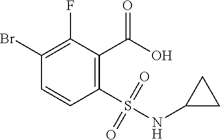 [SMILES: O=C(O)c1c(S(=O)(=O)NC2CC2)ccc(Br)c1F]

To a solution of methyl 3-bromo-6-(N-cyclopropylsulfamoyl)-2-fluorobenzoate (6.6 g, 18.8 mmol) in THF (80 mL) was added LiHMDS (28.2 mL, 28.2 mmol). The mixture was stirred at rt for 2 h. LCMS showed the reaction was complete. The mixture was adjusted to pH=6 with 1 N HCl (aq), then extracted with EA (3×100 mL). The combined organic layers were dried over Na₂SO₄, filtered and concentrated under vacuum to afford product (6.1 g, 96.2%), which was used in the next step directly.

LC-MS (ESI): m/z [M+H]⁺=338.

### Step 3:5-bromo-2-cyclopropyl-4-fluorobenzo[d]isothiazol-3 (2H)-one 1,1-dioxide

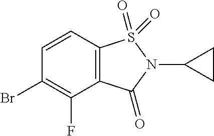 [SMILES: O=C1c2c(ccc(Br)c2F)S(=O)(=O)N1C1CC1]

A solution of 3-bromo-6-(N-cyclopropylsulfamoyl)-2-fluorobenzoic acid (6.1 g, 18.1 mmol) in SOCl₂ (70 mL) was stirred at 75° C. for 3 h. LCMS showed the reaction was complete. The mixture was concentrated under vacuum to afford product (5.5 g, 95.3%), which was used in the next step directly.

LC-MS (ESI): m/z [M+H]⁺=320.

### Step 4:5-bromo-2-cyclopropyl-4-fluoro-2,3-dihydrobenzo[d]isothiazole 1,1-dioxide

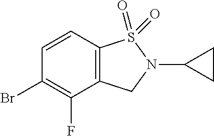 [SMILES: O=S1(=O)c2ccc(Br)c(F)c2CN1C1CC1]

A solution of 5-bromo-2-cyclopropyl-4-fluorobenzo[d]isothiazol-3 (2H)-one 1,1-dioxide (5.5 g, 17.2 mmol) in THF (70 mL) was added BH₃ (86 mL, 86.2 mmol). The mixture was stirred at 75° C. for 16 h. LCMS showed the reaction was complete. The mixture was quenched by MeOH slowly. The mixture was concentrated under vacuum. The residue was purified by silica gel column chromatography, eluting with EA/PE (0-45%) to afford the product (3.2 g, 60.8%). LC-MS (ESI): m/z [M+H]⁺=306.

### Step 5: cis-3-(1-(tert-butyl)-5-((2-cyclopropyl-4-fluoro-1,1-dioxido-2,3-dihydrobenzo[d]isothiazol-5-yl)amino)-1H-pyrazol-3-yl)cyclopentyl isopropylcarbamate

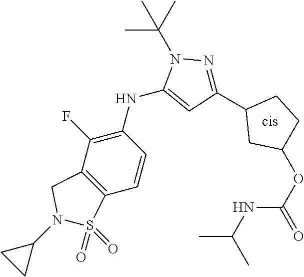 [SMILES: CC(C)NC(=O)OC1CCC(c2cc(Nc3ccc4c(c3F)CN(C3CC3)S4(=O)=O)n(C(C)(C)C)n2)C1]

A mixture of 5-bromo-2-cyclopropyl-4-fluoro-2,3-dihydrobenzo[d]isothiazole 1,1-dioxide (70 mg, 0.23 mmol), cis-3-(5-amino-1-(tert-butyl)-1H-pyrazol-3-yl)cyclopentyl isopropylcarbamate (71 mg, 0.23 mmol), Pd₂(dba)₃ (21 mg, 0.023 mmol), Xantphos (26.6 mg, 0.046 mmol) and K₃PO₄ (146 mg, 0.69 mmol) in dioxane (10 mL) was stirred at 90° C. for 16 h under a nitrogen atmosphere. LCMS showed the reaction was complete. The mixture was concentrated under vacuum. The residue was purified by silica gel column chromatography, eluting with EA/PE (0-80%) to afford the product (104 mg, 85.2%). LC-MS (ESI): m/z [M+H]⁺=534.2.

### Step 6: cis-3-(3-((2-cyclopropyl-4-fluoro-1,1-dioxido-2,3-dihydrobenzo[d]isothiazol-5-yl)amino)-1H-pyrazol-5-yl)cyclopentyl isopropylcarbamate

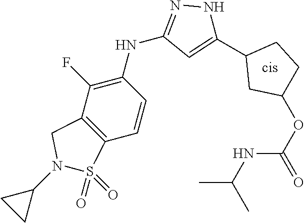 [SMILES: CC(C)NC(=O)OC1CCC(c2cc(Nc3ccc4c(c3F)CN(C3CC3)S4(=O)=O)n[nH]2)C1]

To a solution of 3-(1-(tert-butyl)-5-((2-cyclopropyl-4-fluoro-1,1-dioxido-2,3-dihydrobenzo[d]isothiazol-5-yl)amino)-1H-pyrazol-3-yl)cyclopentyl isopropylcarbamate (104 mg, 0.19 mmol) in DCM (15 mL) was added TfOH (3 mL). The mixture was stirred at rt for 2 h. LCMS showed the reaction was complete. The mixture was quenched by aq NaHCO₃ (50 mL), extracted with DCM (3×50 mL). The combined organic layers were dried over Na₂SO₄, filtered and concentrated under vacuum. The residue was purified by Prep-HPLC to afford desired product in racemic form, which was further separated by Chiral Prep-HPLC to give:

Enantiomer 1 (Example 116a, 100% ee); Retention time: 6.43 min, ¹H NMR (500 MHz, DMSO-d₆) δ 12.00 (s, 1H), 8.77 (s, 1H), 8.28 (t, J=5 Hz, 1H), 7.52 (d, J=10 Hz, 1H), 6.94 (d, J=10 Hz, 1H), 5.83 (s, 1H), 5.02-4.98 (m, 1H), 4.52 (s, 2H), 3.63-3.52 (m, 1H), 3.12-3.00 (m, 1H), 2.48-2.43 (m, 2H), 2.07-1.98 (m, 1H), 1.95-1.85 (m, 1H), 1.78-1.64 (m, 2H), 1.64-1.55 (m, 1H), 1.08-0.97 (m, 6H), 0.87-0.81 (m, 2H), 0.76-0.70 (m, 2H). LC-MS (ESI): m/z [M+H]⁺=478.2.

Enantiomer 2 (Example 116b, 100% ee); Retention time: 11.73 min, ¹H NMR (500 MHz, DMSO-d₆) δ 12.00 (s, 1H), 8.78 (s, 1H), 8.29 (t, J=5 Hz, 1H), 7.52 (d, J=10 Hz, 1H), 6.94 (d, J=10 Hz, 1H), 5.83 (s, 1H), 5.03-4.97 (m, 1H), 4.52 (s, 2H), 3.62-3.52 (m, 1H), 3.12-3.01 (m, 1H), 2.47-2.42 (m, 2H), 2.08-1.98 (m, 1H), 1.97-1.86 (m, 1H), 1.78-1.64 (m, 2H), 1.64-1.55 (m, 1H), 1.08-0.96 (m, 6H), 0.87-0.82 (m, 2H), 0.76-0.71 (m, 2H). LC-MS (ESI): m/z [M+H]⁺=478.3.

Chiral analytical method: Column: I-Cellulose-5, 4.6 mm×250 mm, 5 μm; Mobile phase: A for Hexane and B for EtOH (0.1% 2M NH₃ MeOH); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 μm; Mobile phase: A for Hexane and B for EtOH (0.2% 2M NH₃ MeOH); Gradient: Mobile Phase A: Mobile Phase B=60: 40 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 117: cis-3-(3-((2-cyclopropyl-4-fluoro-1,1-dioxido-2,3-dihydrobenzo[d]isothiazol-5-yl)amino)-1H-pyrazol-5-yl)cyclopentyl((S)-sec-butyl)carbamate

### Step 1: cis-3-(5-amino-1-(tert-butyl)-1H-pyrazol-3-yl)cyclopentan-1-ol

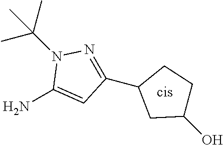 [SMILES: CC(C)(C)n1nc(C2CCC(O)C2)cc1N]

To a solution of cis-benzyl(1-(tert-butyl)-3-(3-hydroxycyclopentyl)-1H-pyrazol-5-yl)carbamate (3.6 g, 10 mmol) in THF (100 mL) was added Pd/C (10%, wet, 2 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (2.1 g, 91% yield). LC-MS (ESI): m/z [M+H]⁺=224.3.

### Step 2: cis-5-((1-(tert-butyl)-3-(3-hydroxycyclopentyl)-1H-pyrazol-5-yl)amino)-2-cyclopropyl-4-fluoro-2,3-dihydrobenzo[d]isothiazole 1,1-dioxide

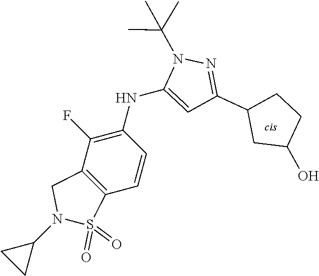 [SMILES: CC(C)(C)n1nc(C2CCC(O)C2)cc1Nc1ccc2c(c1F)CN(C1CC1)S2(=O)=O]

A mixture of 5-bromo-2-cyclopropyl-4-fluoro-2,3-dihydrobenzo[d]isothiazole 1,1-dioxide (140 mg, 0.46 mmol), cis-3-(5-amino-1-(tert-butyl)-1H-pyrazol-3-yl)cyclopentan-1-ol (102.6 mg, 0.46 mmol), Pd₂dba₃ (42 mg, 0.046 mmol), Xantphos (53.2 mg, 0.092 mmol) and K₃PO₄ (293 mg, 1.38 mmol) in dioxane (10 mL) was stirred at 90° C. for 16 h under a nitrogen atmosphere. LCMS showed the reaction was complete. The mixture was concentrated under vacuum. The residue was purified by silica gel column chromatography, eluting with EA/PE (0-80%) to afford the product (114 mg, 55.3%). LC-MS (ESI): m/z [M+H]⁺=449.2.

### Step 3: cis-3-(1-(tert-butyl)-5-((2-cyclopropyl-4-fluoro-1,1-dioxido-2,3-dihydrobenzo[d]isothiazol-5-yl)amino)-1H-pyrazol-3-yl)cyclopentyl(4-nitrophenyl) carbonate

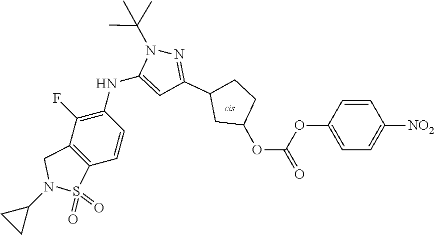 [SMILES: CC(C)(C)n1nc(C2CCC(OC(=O)Oc3ccc([N+](=O)[O-])cc3)C2)cc1Nc1ccc2c(c1F)CN(C1CC1)S2(=O)=O]

To a mixture of cis-5-((1-(tert-butyl)-3-(3-hydroxycyclopentyl)-1H-pyrazol-5-yl)amino)-2-cyclopropyl-4-fluoro-2,3-dihydrobenzo[d]isothiazole 1,1-dioxide (114 mg, 0.25 mmol), DMAP (10 mg, 0.13 mmol) and pyridine (155 mg, 1.27 mmol) in THF (15 mL) was added 4-nitrophenyl carbonochloridate (61.2 mg, 0.3 mmol). The mixture was stirred at 50° C. for 16 h. LCMS showed the reaction was complete. The mixture was concentrated under vacuum. The residue was purified by silica gel column chromatography, eluting with EA/PE (0-55%) to afford the product (80 mg, 73%). LC-MS (ESI): m/z [M+H]⁺=614.3.

### Step 3: cis-3-(3-((2-cyclopropyl-4-fluoro-1,1-dioxido-2,3-dihydrobenzo[d]isothiazol-5-yl)amino)-1H-pyrazol-5-yl)cyclopentyl(4-nitrophenyl) carbonate

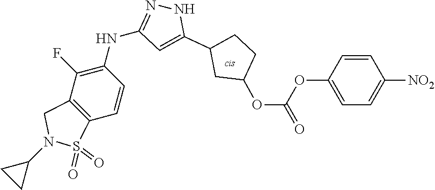 [SMILES: O=C(Oc1ccc([N+](=O)[O-])cc1)OC1CCC(c2cc(Nc3ccc4c(c3F)CN(C3CC3)S4(=O)=O)n[nH]2)C1]

A solution of cis-3-(1-(tert-butyl)-5-((2-cyclopropyl-4-fluoro-1,1-dioxido-2,3-dihydrobenzo[d]isothiazol-5-yl)amino)-1H-pyrazol-3-yl)cyclopentyl(4-nitrophenyl) carbonate (80 mg, 0.13 mmol) in FA (10 mL) was stirred at 75° C. for 1 days. LCMS showed the reaction was complete. The mixture was concentrated under vacuum to afford desired product (75 mg, crude), which was used in the next step directly. LC-MS (ESI): m/z [M+H]⁺=558.2.

### Step 4: cis-3-(3-((2-cyclopropyl-4-fluoro-1,1-dioxido-2,3-dihydrobenzo[d]isothiazol-5-yl)amino)-1H-pyrazol-5-yl)cyclopentyl((S)-sec-butyl)carbamate

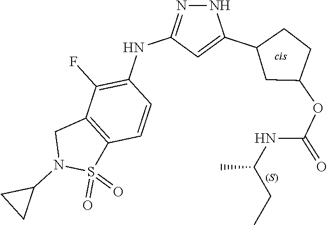

A mixture of cis-3-(3-((2-cyclopropyl-4-fluoro-1,1-dioxido-2,3-dihydrobenzo[d]isothiazol-5-yl)amino)-1H-pyrazol-5-yl)cyclopentyl(4-nitrophenyl) carbonate (25 mg, 0.045 mmol), (S)-butan-2-amine (7.3 mg, 0.067 mmol) and DIEA (17.4 mg, 0.135 mmol) in THF (10 mL) was stirred at 50° C. for 16 h. The mixture was concentrated under vacuum. The residue was purified by Prep-HPLC to afford desired product (3.53 mg, 16.5%) in a racemic form, which was further separated by Chiral Prep-HPLC to give:

Enantiomer 1 (Example 117a, 100% ee); Retention time: 5.67 min. ¹H NMR (500 MHz, DMSO-d₆) δ 12.00 (s, 1H), 8.77 (s, 1H), 8.28 (t, J=10 Hz, 1H), 7.52 (d, J=10 Hz, 1H), 6.88 (d, J=10 Hz, 1H), 5.83 (s, 1H), 5.03-4.96 (m, 1H), 4.52 (s, 2H), 3.42-3.34 (m, 1H), 3.11-3.01 (m, 1H), 2.48-2.43 (m, 2H), 2.09-1.98 (m, 1H), 1.96-1.85 (m, 1H), 1.78-1.65 (m, 2H), 1.63-1.55 (m, 1H), 1.41-1.29 (m, 2H), 1.03-0.96 (m, 3H), 0.87-0.76 (m, 5H), 0.76-0.70 (m, 2H). LC-MS (ESI): m/z [M+H]⁺=492.4.

Enantiomer 2 (example 117b, 100% ee); Retention time: 10.41 min. ¹H NMR (500 MHz, DMSO-d₆) δ 12.00 (s, 1H), 8.77 (s, 1H), 8.28 (t, J=10 Hz, 1H), 7.52 (d, J=10 Hz, 1H), 6.88 (d, J=10 Hz, 1H), 5.83 (s, 1H), 5.03-4.96 (m, 1H), 4.52 (s, 2H), 3.42-3.34 (m, 1H), 3.11-3.01 (m, 1H), 2.48-2.43 (m, 2H), 2.09-1.98 (m, 1H), 1.96-1.85 (m, 1H), 1.78-1.65 (m, 2H), 1.63-1.55 (m, 1H), 1.41-1.29 (m, 2H), 1.03-0.96 (m, 3H), 0.87-0.76 (m, 5H), 0.76-0.70 (m, 2H). LC-MS (ESI): m/z [M+H]⁺=492.4.

Chiral analytical method: Column: CHIRALPAK IE 4.6 mm×250 mm, 5 μm; Mobile phase: A for MtBE (0.1% 2M NH₃ MeOH) (%) and B for EtOH; Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for EtOH; Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 118: cis-3-(3-((2-cyclopropyl-4-fluoro-1,1-dioxido-2,3-dihydrobenzo[d]isothiazol-5-yl)amino)-1H-pyrazol-5-yl)cyclopentyl propylcarbamate

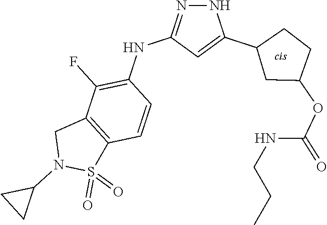 [SMILES: CCCNC(=O)OC1CCC(c2cc(Nc3ccc4c(c3F)CN(C3CC3)S4(=O)=O)n[nH]2)C1]

The titled compound was synthesized in the procedures similar to Example 117 in a racemic form, which was further separated by Chiral Prep-HPLC to give:

Enantiomer 1 (Example 118a, 100% ee); Retention time: 6.27 min. ¹H NMR (500 MHz, DMSO-d₆) δ 12.00 (s, 1H), 8.77 (s, 1H), 8.28 (t, J=10 Hz, 1H), 7.52 (d, J=10 Hz, 1H), 7.04 (t, J=10 Hz, 1H), 5.83 (s, 1H), 5.04-4.96 (m, 1H), 4.52 (s, 2H), 3.12-3.01 (m, 1H), 2.95-2.87 (m, 2H), 2.49-2.43 (m, 2H), 2.06-2.00 (m, 1H), 1.96-1.85 (m, 1H), 1.78-1.65 (m, 2H), 1.65-1.55 (m, 1H), 1.43-1.33 (m, 2H), 0.88-0.77 (m, 5H), 0.76-0.70 (m, 2H). LC-MS (ESI): m/z [M+H]⁺=478.1.

Enantiomer 2 (example 118b, 100% ee); Retention time: 10.50 min. ¹H NMR (500 MHz, DMSO-d₆) δ 12.00 (s, 1H), 8.77 (s, 1H), 8.28 (t, J=10 Hz, 1H), 7.52 (d, J=10 Hz, 1H), 7.04 (t, J=10 Hz, 1H), 5.83 (s, 1H), 5.04-4.96 (m, 1H), 4.52 (s, 2H), 3.12-3.01 (m, 1H), 2.95-2.87 (m, 2H), 2.49-2.43 (m, 2H), 2.06-2.00 (m, 1H), 1.96-1.85 (m, 1H), 1.78-1.65 (m, 2H), 1.65-1.55 (m, 1H), 1.43-1.33 (m, 2H), 0.88-0.77 (m, 5H), 0.76-0.70 (m, 2H). LC-MS (ESI): m/z [M+H]⁺=478.1.

Chiral analytical method: Column: CHIRALPAK IE 4.6 mm×250 mm, 5 μm; Mobile phase: A for MtBE (0.1% 2M NH₃ MeOH) (%) and B for EtOH; Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for EtOH; Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 119: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-methylcyclopropyl)carbamate

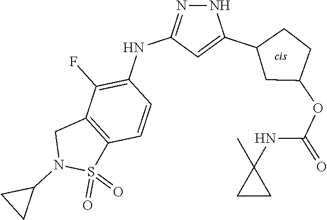 [SMILES: CC1(NC(=O)OC2CCC(c3cc(Nc4ccc5c(c4F)CN(C4CC4)S5(=O)=O)n[nH]3)C2)CC1]

The titled compound was synthesized in the procedures similar to Example 117 in a racemic form, which was further separated by Chiral Prep-HPLC to give:

Enantiomer 1 (Example 119a, 100% ee); Retention time: 6.27 min. ¹H NMR (500 MHz, DMSO-d₆) δ 11.99 (s, 1H), 8.77 (s, 1H), 8.28 (t, J=10 Hz, 1H), 7.52 (d, J=10 Hz, 1H), 7.36-7.52 (m, 1H), 5.82 (s, 1H), 5.06-4.95 (m, 1H), 4.52 (s, 2H), 3.12-3.00 (m, 1H), 2.48-2.44 (m, 2H), 2.09-1.97 (m, 1H), 1.95-1.84 (m, 1H), 1.76-1.63 (m, 2H), 1.61-1.52 (m, 1H), 1.23 (s, 3H), 0.88-0.81 (m, 2H), 0.77-0.70 (m, 2H), 0.62-0.55 (m, 2H), 0.50-0.44 (m, 2H). LC-MS (ESI): m/z [M+H]⁺=490.2.

Enantiomer 2 (example 119b, 100% ee); Retention time: 10.50 min. ¹H NMR (500 MHz, DMSO-d₆) δ 11.99 (s, 1H), 8.77 (s, 1H), 8.28 (t, J=10 Hz, 1H), 7.52 (d, J=10 Hz, 1H), 7.36-7.52 (m, 1H), 5.82 (s, 1H), 5.06-4.95 (m, 1H), 4.52 (s, 2H), 3.12-3.00 (m, 1H), 2.48-2.44 (m, 2H), 2.09-1.97 (m, 1H), 1.95-1.84 (m, 1H), 1.76-1.63 (m, 2H), 1.61-1.52 (m, 1H), 1.23 (s, 3H), 0.88-0.81 (m, 2H), 0.77-0.70 (m, 2H), 0.62-0.55 (m, 2H), 0.50-0.44 (m, 2H). LC-MS (ESI): m/z [M+H]⁺=490.2.

Chiral analytical method: Column: CHIRALPAK IE 4.6 mm×250 mm, 5 μm; Mobile phase: A for MtBE (0.1% 2M NH₃ MeOH) (%) and B for EtOH; Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for EtOH; Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 120: cis-3-(3-((2-cyclopropyl-4-fluoro-1,1-dioxido-2,3-dihydrobenzo[d]isothiazol-5-yl)amino)-1H-pyrazol-5-yl)cyclopentyl((S)-1-hydroxypropan-2-yl)carbamate

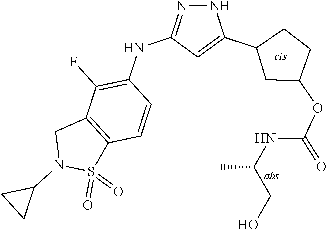

The titled compound was synthesized in the procedures similar to Example 117. ¹H NMR (500 MHz, DMSO-d₆) δ 12.00 (s, 1H), 8.77 (s, 1H), 8.28 (brs, 1H), 7.52 (d, J=10 Hz, 1H), 6.83-6.76 (m, 1H), 5.83 (s, 1H), 5.05-4.97 (m, 1H), 4.65-4.58 (m, 1H), 4.52 (s, 2H), 3.53-3.43 (m, 1H), 3.35-3.33 (m, 1H), 3.30-3.28 (m, 1H), 3.21-3.14 (m, 1H), 3.11-3.02 (m, 1H), 3.48-3.44 (m, 1H), 2.06-1.99 (m, 1H), 1.96-1.86 (m, 1H), 1.79-1.65 (m, 2H), 1.64-1.56 (m, 1H), 1.04-0.96 (m, 3H), 0.84-0.81 (m, 2H), 0.76-0.70 (m, 2H). LC-MS (ESI): m/z [M+H]⁺=494.2.

### Example 121: cis-3-(3-((4-fluoro-2-methyl-1,1-dioxido-2,3-dihydrobenzo[d]isothiazol-5-yl)amino)-1H-pyrazol-5-yl)cyclopentyl isopropylcarbamate

### Step 1:5-bromo-4-fluoro-2-methyl-2,3-dihydrobenzo[d]isothiazole 1,1-dioxide

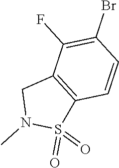

To a mixture of 5-bromo-4-fluoro-2,3-dihydrobenzo[d]isothiazole 1,1-dioxide (266 mg, 1 mmol) and K₂CO₃ (166 mg, 1.2 mmol) in DMF (10 mL) was added CH₃I (170 mg, 1.2 mmol). The mixture was stirred at room temperature for 2 h. LCMS showed the reaction was complete. The mixture was diluted with water (50 mL), extracted with EA (3×50 mL). The combined organic layers were dried over Na₂SO₄, filtered and concentrated under vacuum. The residue was purified by silica gel column chromatography, eluting with PE/EA (3:2) to afford the product (196 mg, 70%). LC-MS (ESI): m/z [M+H]⁺=280.2.

### Step 2: cis-3-(3-((4-fluoro-2-methyl-1,1-dioxido-2,3-dihydrobenzo[d]isothiazol-5-yl)amino)-1H-pyrazol-5-yl)cyclopentyl isopropylcarbamate

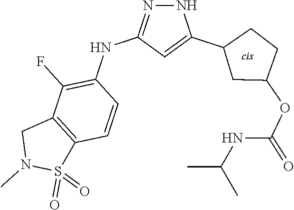 [SMILES: CC(C)NC(=O)OC1CCC(c2cc(Nc3ccc4c(c3F)CN(C)S4(=O)=O)n[nH]2)C1]

The titled compound was synthesized in the procedures similar to Example 34, step 5 in racemic form, which was further separated by Chiral Prep-HPLC to give:

Enantiomer 1 (Example 121a, 100% ee); Retention time: 13.25 min. ¹H NMR (500 MHz, DMSO-d₆) δ 12.01 (s, 1H), 8.78 (s, 1H), 8.29 (s, 1H), 7.55 (d, J=8.7 Hz, 1H), 6.95 (d, J=6.8 Hz, 1H), 5.84 (s, 1H), 5.00 (s, 1H), 4.43 (s, 2H), 3.62-3.55 (m, 1H), 3.12-3.02 (m, 1H), 2.79 (s, 3H), 2.48-2.42 (m, 1H), 2.07-1.95 (m, 1H), 1.95-1.85 (m, 1H), 1.78-1.66 (m, 2H), 1.65-1.56 (m, 1H), 1.03 (d, J=6.2 Hz, 6H). LC-MS (ESI): m/z [M+H]⁺=452.2.

Enantiomer 2 (Example 121b, 100% ee); Retention time: 14.99 min.¹H NMR (500 MHz, DMSO-d₆) δ 12.01 (s, 1H), 8.78 (s, 1H), 8.29 (s, 1H), 7.55 (d, J=8.7 Hz, 1H), 6.95 (d, J=6.8 Hz, 1H), 5.84 (s, 1H), 5.00 (s, 1H), 4.43 (s, 2H), 3.62-3.55 (m, 1H), 3.12-3.02 (m, 1H), 2.79 (s, 3H), 2.48-2.42 (m, 1H), 2.07-1.95 (m, 1H), 1.95-1.85 (m, 1H), 1.78-1.66 (m, 2H), 1.65-1.56 (m, 1H), 1.03 (d, J=6.2 Hz, 6H). LC-MS (ESI): m/z [M+H]⁺=452.2.

Chiral analytical method: Column: CHIRALPAK i-cellulose-5 4.6 mm×250 mm, 5 μm; Mobile phase: A for Hexane and B for EtOH (0.1% 2M NH₃ MeOH); Gradient: Mobile Phase A: Mobile Phase B=70: 30 (v/v); HPLC Equipment: HPLC-Agilent, Back pressure: 100 bar; Column temperature: 35° C.

Chiral Prep-HPLC Condition: CHIRALPAK i-cellulose-5 20 mm×250 mm, 5 μm; Mobile phase: A for Hexane and B for EtOH (0.2% 2M NH₃ MeOH); Gradient: Mobile Phase A: Mobile Phase B=70: 30 (v/v); Flow Rate: 18 mL/min, Wavelength: UV 200 nm and 270 nm, Prep-HPLC Equipment: Prep-HPLC-Gilson, Back pressure: 100 bar; Column temperature: 25° C.

### Example 122: cis-3-(3-((4-fluoro-2-methyl-1,1-dioxido-2,3-dihydrobenzo[d]isothiazol-5-yl)amino)-1H-pyrazol-5-yl)cyclopentyl propylcarbamate

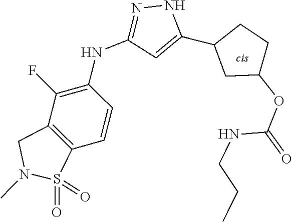 [SMILES: CCCNC(=O)OC1CCC(c2cc(Nc3ccc4c(c3F)CN(C)S4(=O)=O)n[nH]2)C1]

The titled compound was synthesized in the procedures similar to Example 121 in racemic form, which was further purified with chiral-Prep-HPLC to give:

Enantiomer 1 (Example 122a, 100% ee); Retention time: 6.266 min. ¹H NMR (500 MHz, DMSO-d₆) δ 12.00 (s, 1H), 8.77 (s, 1H), 8.28 (s, 1H), 7.54 (d, J=8.6 Hz, 1H), 7.04 (s, 1H), 5.83 (s, 1H), 5.05-4.93 (m, 1H), 4.43 (s, 2H), 3.14-3.02 (m, 1H), 2.96-2.86 (m, 2H), 2.78 (s, 3H), 2.48-2.40 (m, 1H), 2.07-1.99 (m, 1H), 1.98-1.85 (m, 1H), 1.80-1.66 (m, 2H), 1.65-1.55 (m, 1H), 1.44-1.32 (m, 2H), 0.82 (t, J=7.4 Hz, 3H). LC-MS (ESI): m/z [M+H]⁺=452.60.

Enantiomer 2 (Example 122b, 100% ee); Retention time: 11.396 min. ¹H NMR (500 MHz, DMSO-d₆) δ 12.00 (s, 1H), 8.77 (s, 1H), 8.28 (s, 1H), 7.54 (d, J=8.6 Hz, 1H), 7.04 (s, 1H), 5.83 (s, 1H), 5.05-4.93 (m, 1H), 4.43 (s, 2H), 3.14-3.02 (m, 1H), 2.96-2.86 (m, 2H), 2.78 (s, 3H), 2.48-2.40 (m, 1H), 2.07-1.99 (m, 1H), 1.98-1.85 (m, 1H), 1.80-1.66 (m, 2H), 1.65-1.55 (m, 1H), 1.44-1.32 (m, 2H), 0.82 (t, J=7.4 Hz, 3H). LC-MS (ESI): m/z [M+H]⁺=452.60.

Chiral analytical method: Column: CHIRALPAK i-cellulose-5 4.6 mm×250 mm, 5 μm; Mobile phase: A for MTBE (0.1% 2M NH₃ MeOH) and B for MeOH/DCM (1/1); Gradient: Mobile Phase A: Mobile Phase B=70: 30 (v/v); HPLC Equipment: HPLC-Agilent, Back pressure: 100 bar; Column temperature: 35° C.

Chiral Prep-HPLC Condition: CHIRALPAK i-cellulose-5 20 mm×250 mm, 5 μm; Mobile phase: A for Hexane and B for EtOH (0.2% 2M NH₃ MeOH); Gradient: Mobile Phase A: Mobile Phase B=70: 30 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 123: cis-3-(3-((4-fluoro-2-methyl-1,1-dioxido-2,3-dihydrobenzo[d]isothiazol-5-yl)amino)-1H-pyrazol-5-yl)cyclopentyl((S)-sec-butyl)carbamate

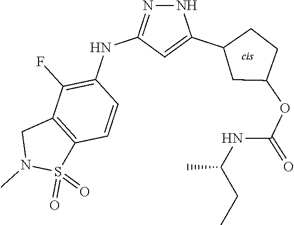 [SMILES: CC[C@H](C)NC(=O)OC1CCC(c2cc(Nc3ccc4c(c3F)CN(C)S4(=O)=O)n[nH]2)C1]

The titled compound was synthesized in the procedures similar to Example 121 in racemic form, which was further purified with chiral-Prep-HPLC to give:

Enantiomer 1 (Example 122a, 100% ee); Retention time: 5.514 min. ¹H NMR (500 MHz, DMSO-d₆) δ 11.99 (s, 1H), 8.76 (s, 1H), 8.27 (s, 1H), 7.54 (d, J=8.6 Hz, 1H), 6.87 (d, J=6.9 Hz, 1H), 5.83 (s, 1H), 4.99 (s, 1H), 4.42 (s, 2H), 3.45-3.35 (m, 1H), 3.10-3.05 (m, 1H), 2.78 (s, 3H), 2.47-2.42 (m, 1H), 2.05-1.85 (m, 2H), 1.78-1.53 (m, 3H), 1.40-1.28 (m, 2H), 1.03-0.96 (m, 3H), 0.80 (q, J=7.1 Hz, 3H). LC-MS (ESI): m/z [M+H]⁺=466.2.

Enantiomer 2 (Example 122b, 100% ee); Retention time: 11.385 min. ¹H NMR (500 MHz, DMSO-d₆) δ 11.99 (s, 1H), 8.76 (s, 1H), 8.27 (s, 1H), 7.54 (d, J=8.6 Hz, 1H), 6.87 (d, J=6.9 Hz, 1H), 5.83 (s, 1H), 4.99 (s, 1H), 4.42 (s, 2H), 3.45-3.35 (m, 1H), 3.10-3.05 (m, 1H), 2.78 (s, 3H), 2.47-2.42 (m, 1H), 2.05-1.85 (m, 2H), 1.78-1.53 (m, 3H), 1.40-1.28 (m, 2H), 1.03-0.96 (m, 3H), 0.80 (q, J=7.1 Hz, 3H). LC-MS (ESI): m/z [M+H]⁺=466.2.

Chiral analytical method: Column: CHIRALPAK i-cellulose-5 4.6 mm×250 mm, 5 μm; Mobile phase: A for MTBE (0.1% 2M NH₃ MeOH) and B for MeOH/DCM (1/1); Gradient: Mobile Phase A: Mobile Phase B=70: 30 (v/v); HPLC Equipment: HPLC-Agilent, Back pressure: 100 bar; Column temperature: 35° C.

Chiral Prep-HPLC Condition: CHIRALPAK i-cellulose-5 20 mm×250 mm, 5 μm; Mobile phase: A for Hexane and B for EtOH (0.2% 2M NH₃ MeOH); Gradient: Mobile Phase A: Mobile Phase B=70: 30 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 124: cis-3-(3-((4-fluoro-2-methyl-1,1-dioxido-2,3-dihydrobenzo[d]isothiazol-5-yl)amino)-1H-pyrazol-5-yl)cyclopentyl(1-methylcyclopropyl)carbamate

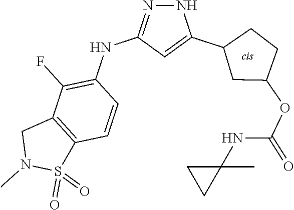 [SMILES: CN1Cc2c(ccc(Nc3cc(C4CCC(OC(=O)NC5(C)CC5)C4)[nH]n3)c2F)S1(=O)=O]

The titled compound was synthesized in the procedures similar to Example 121. ¹H NMR (500 MHz, DMSO-d₆) δ 11.99 (s, 1H), 8.76 (s, 1H), 8.27 (s, 1H), 7.54 (d, J=8.6 Hz, 1H), 7.33 (s, 1H), 5.83 (s, 1H), 4.99 (s, 1H), 4.42 (s, 2H), 3.10-3.02 (m, 1H), 2.78 (s, 3H), 2.46-2.42 (m, 1H), 2.10-1.85 (m, 2H), 1.78-1.55 (m, 3H), 1.23 (s, 3H), 0.59 (s, 2H), 0.47 (s, 2H). LC-MS (ESI): m/z [M+H]⁺=464.2.

### Example 125: cis-3-(3-((4-fluoro-2-(2-hydroxyethyl)-1,1-dioxido-2,3-dihydrobenzo[d]isothiazol-5-yl)amino)-1H-pyrazol-5-yl)cyclopentyl isopropylcarbamate

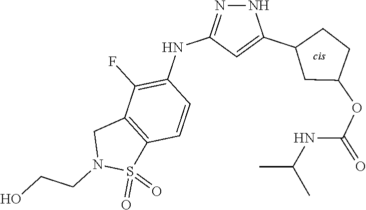 [SMILES: CC(C)NC(=O)OC1CCC(c2cc(Nc3ccc4c(c3F)CN(CCO)S4(=O)=O)n[nH]2)C1]

The titled compound was synthesized in the procedures similar to Example 37 in racemic form, which was further separated by Chiral Prep-HPLC to give:

Enantiomer 1 (Example 125a, 100% ee); Retention time: 14.34 min.¹H NMR (500 MHz, DMSO-d₆) δ 12.00 (s, 1H), 8.77 (s, 1H), 8.29 (d, J=7.7 Hz, 1H), 7.54 (d, J=8.6 Hz, 1H), 6.94 (d, J=7.4 Hz, 1H), 5.83 (s, 1H), 4.99 (s, 1H), 4.91 (t, J=5.6 Hz, 1H), 4.54 (s, 2H), 3.68 (q, J=5.7 Hz, 2H), 3.62-3.54 (m, 1H), 3.21 (t, J=5.8 Hz, 2H), 3.12-3.01 (m, 1H), 2.29-2.23 (m, 1H), 2.05-1.96 (m, 1H), 1.96-1.86 (m, 1H), 1.75-1.65 (m, 2H), 1.64-1.55 (m, 1H), 1.03 (d, J=6.1 Hz, 7H). LC-MS (ESI): m/z [M+H]⁺=482.2.

Enantiomer 2 (Example 125b, 98.8% ee); Retention time: 15.95 min. ¹H NMR (500 MHz, DMSO-d₆) δ 12.00 (s, 1H), 8.78 (s, 1H), 8.30 (t, J=7.8 Hz, 1H), 7.54 (d, J=8.6 Hz, 1H), 6.95 (d, J=7.8 Hz, 1H), 5.83 (s, 1H), 5.00 (s, 1H), 4.92 (t, J=5.5 Hz, 1H), 4.54 (s, 2H), 3.68 (q, J=5.7 Hz, 2H), 3.62-3.56 (m, 1H), 3.21 (t, J=5.8 Hz, 2H), 3.10-3.01 (m, 1H), 2.47-2.43 (m, 1H), 2.07-1.87 (m, 2H), 1.78-1.55 (m, 3H), 1.03 (d, J=6.0 Hz, 6H). LC-MS (ESI): m/z [M+H]⁺=482.2.

Chiral analytical method: Column: CHIRALPAK i-cellulose-5 4.6 mm×250 mm, 5 μm; Mobile phase: A for Hexane and B for EtOH (0.1% 2M NH₃ MeOH); Gradient: Mobile Phase A: Mobile Phase B=70: 30 (v/v); HPLC Equipment: HPLC-Agilent, Back pressure: 100 bar; Column temperature: 35° C.

Chiral Prep-HPLC Condition: CHIRALPAK i-cellulose-5 20 mm×250 mm, 5 μm; Mobile phase: A for Hexane and B for EtOH (0.2% 2M NH₃ MeOH); Gradient: Mobile Phase A: Mobile Phase B=70: 30 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 126: cis-3-(3-((4-fluoro-2-(2-hydroxyethyl)-1,1-dioxido-2,3-dihydrobenzo[d]isothiazol-5-yl)amino)-1H-pyrazol-5-yl)cyclopentyl propylcarbamate

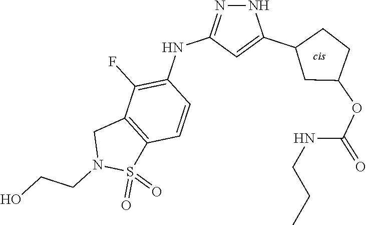 [SMILES: CCCNC(=O)OC1CCC(c2cc(Nc3ccc4c(c3F)CN(CCO)S4(=O)=O)n[nH]2)C1]

The titled compound was synthesized in the procedures similar to Example 37. ¹H NMR (500 MHz, DMSO-d₆) δ=12.00 (s, 1H), 8.76 (s, 1H), 8.28 (s, 1H), 7.54 (d, J=8.6, 1H), 7.03 (t, J=5.1, 1H), 5.84 (s, 1H), 5.00 (m, 1H), 4.54 (s, 2H), 3.68 (t, J=5.8, 2H), 3.21 (t, J=5.8, 2H), 3.11-3.02 (m, 1H), 2.92 (m, 2H), 2.47-2.41 (m, 1H), 2.03 (m, 1H), 1.98-1.83 (m, 1H), 1.81-1.65 (m, 2H), 1.65-1.54 (m, 1H), 1.39 (m, 2H), 0.82 (t, J=7.4, 3H). LC-MS (ESI): m/z [M+H]⁺=482.2.

### Example 127: cis-3-(3-((4-fluoro-2-(2-hydroxyethyl)-1,1-dioxido-2,3-dihydrobenzo[d]isothiazol-5-yl)amino)-1H-pyrazol-5-yl)cyclopentyl((S)-sec-butyl)carbamate

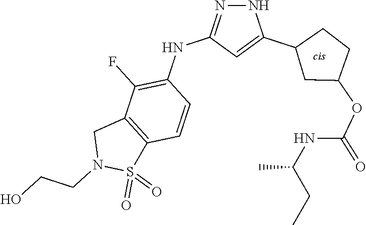 [SMILES: CC[C@H](C)NC(=O)OC1CCC(c2cc(Nc3ccc4c(c3F)CN(CCO)S4(=O)=O)n[nH]2)C1]

The titled compound was synthesized in the procedures similar to Example 37. ¹H NMR (500 MHz, DMSO-d₆) δ=11.99 (s, 1H), 8.76 (s, 1H), 8.27 (s, 1H), 7.53 (d, J=8.6, 1H), 6.87 (d, J=7.9, 1H), 5.84 (s, 1H), 4.99 (s, 1H), 4.54 (s, 2H), 3.68 (t, J=5.8, 2H), 3.39 (m, 1H), 3.21 (t, J=5.8, 2H), 3.11-3.03 (m, 1H), 2.47-2.43 (m, 1H), 2.03 (m, 1H), 1.93 (m, 1H), 1.73 (m, 2H), 1.60 (m, 1H), 1.35 (m, 2H), 1.05-0.93 (m, 3H), 0.80 (m, 3H). LC-MS (ESI): m/z [M+H]⁺=496.3.

### Example 128: cis-3-(3-((4-fluoro-2-(2-hydroxyethyl)-1,1-dioxido-2,3-dihydrobenzo[d]isothiazol-5-yl)amino)-1H-pyrazol-5-yl)cyclopentyl(1-methylcyclopropyl)carbamate

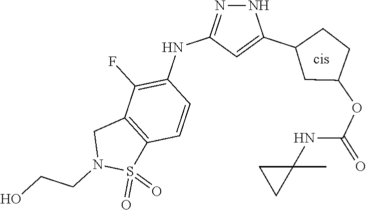 [SMILES: CC1(NC(=O)OC2CCC(c3cc(Nc4ccc5c(c4F)CN(CCO)S5(=O)=O)n[nH]3)C2)CC1]

The titled compound was synthesized in the procedures similar to Example 37 in a racemic form, which was further separated by Chiral Prep-HPLC to give:

Enantiomer 1 (Example 128a, 100% ee); Retention time: 6.156 min. ¹H NMR (500 MHz, DMSO-d₆) δ=11.99 (s, 1H), 8.77 (s, 1H), 8.29 (t, J=8.0, 1H), 7.54 (d, J=8.6, 1H), 7.34 (s, 1H), 5.82 (s, 1H), 4.99 (m, 1H), 4.91 (t, J=5.5, 1H), 4.54 (s, 2H), 3.68 (q, J=5.7, 2H), 3.21 (t, J=5.8, 2H), 3.06 (m, 1H), 2.46 (m, 1H), 2.02 (m, 1H), 1.90 (m, 1H), 1.69 (m, 2H), 1.57 (m, 1H), 1.23 (s, 3H), 0.57 (m, 2H), 0.53-0.40 (m, 2H). LC-MS (ESI): m/z [M+H]⁺=494.28.

Enantiomer 2 (Example 128b, 100% ee); Retention time: 11.817 min. ¹H NMR (500 MHz, DMSO-d₆) δ=11.99 (s, 1H), 8.77 (s, 1H), 8.29 (s, 1H), 7.54 (d, J=8.6, 1H), 7.34 (s, 1H), 5.83 (s, 1H), 4.99 (m, 1H), 4.91 (t, J=5.5, 1H), 4.54 (s, 2H), 3.68 (q, J=5.7, 2H), 3.21 (t, J=5.8, 2H), 3.05 (m, 1H), 2.47 (m, 1H), 2.02 (m, 1H), 1.88 (m, 1H), 1.69 (m, 2H), 1.57 (m, 1H), 1.23 (s, 3H), 0.59 (m, 2H), 0.53-0.43 (m, 2H). LC-MS (ESI): m/z [M+H]⁺=494.26.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=20: 80 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=20: 80 (v/v); Flow Rate: 18 mL/min, Wavelength: UV 280 nm and 300 nm, Prep-HPLC Equipment: Prep-HPLC-Gilson; Column temperature: 25° C.

### Example 129: cis-3-(3-((4-fluoro-2-(methyl-d3)-1,1-dioxido-2,3-dihydrobenzo[d]isothiazol-5-yl)amino)-1H-pyrazol-5-yl)cyclopentyl isopropylcarbamate

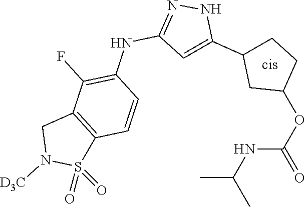 [SMILES: [2H]C([2H])([2H])N1Cc2c(ccc(Nc3cc(C4CCC(OC(=O)NC(C)C)C4)[nH]n3)c2F)S1(=O)=O]

The titled compound was synthesized in the procedures similar to Example 35 in racemic form, which was further separated by Chiral Prep-HPLC to give:

Enantiomer 1 (Example 129a, 100% ee); Retention time: 5.906 min.¹H NMR (500 MHz, DMSO-d₆) δ 12.00 (s, 1H), 8.77 (s, 1H), 8.28 (t, J=7.8 Hz, 1H), 7.54 (d, J=8.6 Hz, 1H), 6.94 (d, J=7.3 Hz, 1H), 5.83 (s, 1H), 4.99 (s, 1H), 4.42 (s, 2H), 3.64-3.47 (m, 1H), 3.12-3.01 (m, 1H), 2.48-2.43 (m, 1H), 2.07-1.85 (m, 2H), 1.77-1.56 (m, 3H), 1.10-0.96 (m, 6H). LC-MS (ESI): m/z [M+H]⁺=455.2.

Enantiomer 2 (Example 129b, 100% ee); Retention time: 1.718 min. ¹H NMR (500 MHz, DMSO-d₆) δ 12.00 (s, 1H), 8.77 (s, 1H), 8.28 (t, J=7.9 Hz, 1H), 7.54 (d, J=8.6 Hz, 1H), 6.94 (d, J=7.3 Hz, 1H), 5.83 (s, 1H), 5.00 (s, 1H), 4.42 (s, 2H), 3.62-3.53 (m, 1H), 3.11-3.03 (m, 1H), 2.48-2.43 (m, 1H), 2.07-1.85 (m, 2H), 1.77-1.56 (m, 3H), 1.10-0.96 (m, 6H). LC-MS (ESI): m/z [M+H]⁺=455.2.

Chiral analytical method: Column: CHIRALPAK IE 4.6 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 130: cis-3-(3-((4-fluoro-2-(methyl-d3)-1,1-dioxido-2,3-dihydrobenzo[d]isothiazol-5-yl)amino)-1H-pyrazol-5-yl)cyclopentyl propylcarbamate

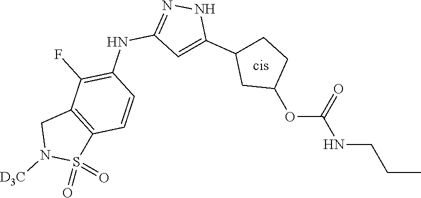 [SMILES: [2H]C([2H])([2H])N1Cc2c(ccc(Nc3cc(C4CCC(OC(=O)NCCC)C4)[nH]n3)c2F)S1(=O)=O]

The titled compound was synthesized in the procedures similar to Example 35. ¹H NMR (500 MHz, DMSO-d₆) δ 12.00 (s, 1H), 8.77 (s, 1H), 8.28 (s, 1H), 7.54 (d, J=8.6 Hz, 1H), 7.04 (s, 1H), 5.83 (s, 1H), 5.00 (s, 1H), 4.42 (s, 2H), 3.12-3.03 (m, 1H), 2.91 (dd, J=13.1, 6.6 Hz, 2H), 2.48-2.43 (m, 1H), 2.06-1.88 (m, 2H), 1.78-1.55 (m, 3H), 1.39 (dd, J=14.5, 7.2 Hz, 2H), 0.82 (t, J=7.4 Hz, 3H).

LC-MS (ESI): m/z [M+H]⁺=455.3.

### Example 131: cis-3-(3-((4-fluoro-2-(methyl-d3)-1,1-dioxido-2,3-dihydrobenzo[d]isothiazol-5-yl)amino)-1H-pyrazol-5-yl)cyclopentyl((S)-sec-butyl)carbamate

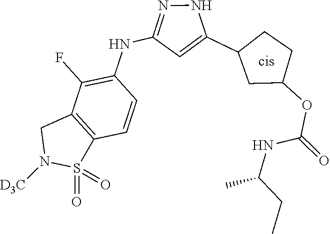 [SMILES: [2H]C([2H])([2H])N1Cc2c(ccc(Nc3cc(C4CCC(OC(=O)N[C@@H](C)CC)C4)[nH]n3)c2F)S1(=O)=O]

The titled compound was synthesized in the procedures similar to Example 35. ¹H NMR (500 MHz, DMSO-d₆) δ 12.00 (s, 1H), 8.77 (s, 1H), 8.28 (s, 1H), 7.54 (d, J=8.6 Hz, 1H), 6.88 (d, J=8.6 Hz, 1H), 5.84 (s, 1H), 4.99 (s, 1H), 4.42 (s, 2H), 3.44-3.35 (m, 1H), 3.12-3.02 (m, 1H), 2.46-2.44 (m, 1H), 2.06-1.85 (m, 2H), 1.77-1.56 (m, 3H), 1.41-1.26 (m, 2H), 1.02-0.96 (m, 3H), 0.80 (q, J=7.2 Hz, 3H). LC-MS (ESI): m/z [M+H]⁺=469.2.

### Example 132: cis-3-(3-((4-fluoro-2-(methyl-d3)-1,1-dioxido-2,3-dihydrobenzo[d]isothiazol-5-yl)amino)-1H-pyrazol-5-yl)cyclopentyl(1-methylcyclopropyl)carbamate

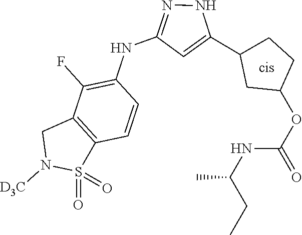 [SMILES: [2H]C([2H])([2H])N1Cc2c(ccc(Nc3cc(C4CCC(OC(=O)N[C@@H](C)CC)C4)[nH]n3)c2F)S1(=O)=O]

The titled compound was synthesized in the procedures similar to Example 35 in a racemic form, which was further separated by Chiral Prep-HPLC to give:

Enantiomer 1 (Example 132a, 100% ee); Retention time: 6.279 min. ¹H NMR (500 MHz, DMSO-d₆) δ=12.00 (s, 1H), 8.77 (s, 1H), 8.28 (t, J=8.0, 1H), 7.54 (d, J=8.6, 1H), 7.34 (s, 1H), 5.83 (s, 1H), 4.99 (m, 1H), 4.42 (s, 2H), 3.06 (m, 1H), 2.46 (m, 1H), 2.02 (m, 1H), 1.97-1.85 (m, 1H), 1.69 (m, 2H), 1.57 (m, 1H), 1.23 (s, 3H), 0.59 (m, 2H), 0.51-0.44 (m, 2H). LC-MS (ESI): m/z [M+H]⁺=467.33.

Enantiomer 2 (Example 132b, 100% ee); Retention time: 10.24 min. ¹H NMR (500 MHz, DMSO-d₆) δ=12.00 (s, 1H), 8.77 (s, 1H), 8.28 (t, J=7.8, 1H), 7.54 (d, J=8.6, 1H), 7.34 (s, 1H), 5.83 (s, 1H), 4.99 (m, 1H), 4.42 (s, 2H), 3.06 (m, 1H), 2.46 (m, 1H), 2.02 (m, 1H), 1.90 (m, 1H), 1.69 (m, 2H), 1.57 (m, 1H), 1.23 (s, 3H), 0.57 (m, 2H), 0.53-0.41 (m, 2H). LC-MS (ESI): m/z [M+H]⁺=467.34.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=20: 80 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=20: 80 (v/v); Flow Rate: 18 mL/min, Wavelength: UV 280 nm and 300 nm, Prep-HPLC Equipment: Prep-HPLC-Gilson; Column temperature: 25° C.

### Example 133: cis-3-(3-((4-fluoro-2-(oxetan-3-yl)-1,1-dioxido-2,3-dihydrobenzo[d]isothiazol-5-yl)amino)-1H-pyrazol-5-yl)cyclopentyl isopropylcarbamate

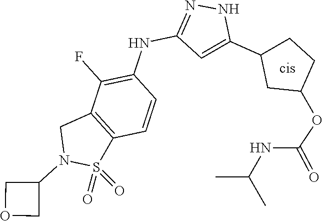 [SMILES: CC(C)NC(=O)OC1CCC(c2cc(Nc3ccc4c(c3F)CN(C3COC3)S4(=O)=O)n[nH]2)C1]

The titled compound was synthesized in the procedures similar to Example 63 in racemic form, which was further separated by Chiral Prep-HPLC to give:

Enantiomer 1 (Example 133a, 100% ee); Retention time: 5.684 min. ¹H NMR (500 MHz, DMSO-d₆) δ 12.01 (s, 1H), 8.84 (s, 1H), 8.32 (t, J=8.1 Hz, 1H), 7.56 (d, J=8.7 Hz, 1H), 6.94 (d, J=6.8 Hz, 1H), 5.84 (d, J=1.9 Hz, 1H), 5.00 (s, 1H), 4.87 (t, J=6.1 Hz, 2H), 4.76 (dq, J=8.2, 6.3 Hz, 5H), 3.62-3.53 (m, 1H), 3.12-3.03 (m, 1H), 2.48-2.43 (m, 1H), 2.06-1.87 (m, 2H), 1.78-1.55 (m, 3H), 1.03 (d, J=6.2 Hz, 6H). LC-MS (ESI): m/z [M+H]⁺=494.2.

Enantiomer 2 (Example 133b, 100% ee); Retention time: 10.123 min. ¹H NMR (500 MHz, DMSO-d₆) δ 12.01 (s, 1H), 8.84 (s, 1H), 8.32 (t, J=8.1 Hz, 1H), 7.56 (d, J=8.7 Hz, 1H), 6.94 (d, J=6.8 Hz, 1H), 5.84 (d, J=1.9 Hz, 1H), 5.00 (s, 1H), 4.87 (t, J=6.1 Hz, 2H), 4.76 (dq, J=8.2, 6.3 Hz, 5H), 3.62-3.53 (m, 1H), 3.12-3.03 (m, 1H), 2.48-2.43 (m, 1H), 2.06-1.87 (m, 2H), 1.78-1.55 (m, 3H), 1.03 (d, J=6.2 Hz, 6H). LC-MS (ESI): m/z [M+H]⁺=494.2.

Chiral analytical method: Column: CHIRALPAK IE 4.6 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 134: cis-3-(3-((4-fluoro-2-(oxetan-3-yl)-1,1-dioxido-2,3-dihydrobenzo[d]isothiazol-5-yl)amino)-1H-pyrazol-5-yl)cyclopentyl((S)-sec-butyl)carbamate

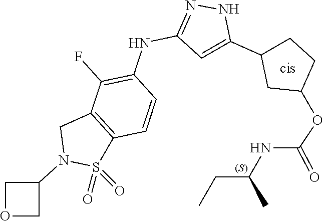

The titled compound was synthesized in the procedures similar to Example 63 in racemic form, which was further separated by Chiral Prep-HPLC to give:

Enantiomer 1 (Example 134a, 100% ee); Retention time: 5.451 min. ¹H NMR (500 MHz, DMSO-d₆) δ 12.01 (s, 1H), 8.84 (s, 1H), 8.31 (s, 1H), 7.55 (d, J=8.7 Hz, 1H), 6.88 (d, J=7.8 Hz, 1H), 5.84 (s, 1H), 4.99 (s, 1H), 4.87 (t, J=6.1 Hz, 2H), 4.81-4.72 (m, 5H), 3.43-3.35 (m, 1H), 3.10-3.02 (m, 1H), 2.47-2.45 (m, 1H), 2.10-1.85 (m, 2H), 1.78-1.55 (m, 3H), 1.40-1.28 (m 2H), 1.04-0.98 (m, 3H), 0.80 (q, J=7.2 Hz, 3H). LC-MS (ESI): m/z [M+H]⁺=508.3.

Enantiomer 2 (Example 134b, 100% ee); Retention time: 10.665 min. ¹H NMR (500 MHz, DMSO-d₆) δ 12.01 (s, 1H), 8.84 (s, 1H), 8.31 (s, 1H), 7.55 (d, J=8.7 Hz, 1H), 6.88 (d, J=7.8 Hz, 1H), 5.84 (s, 1H), 4.99 (s, 1H), 4.87 (t, J=6.1 Hz, 2H), 4.81-4.72 (m, 5H), 3.43-3.35 (m, 1H), 3.10-3.02 (m, 1H), 2.47-2.45 (m, 1H), 2.10-1.85 (m, 2H), 1.78-1.55 (m, 3H), 1.40-1.28 (m 2H), 1.04-0.98 (m, 3H), 0.80 (q, J=7.2 Hz, 3H). LC-MS (ESI): m/z [M+H]⁺=508.3.

Chiral analytical method: Column: CHIRALPAK IE 4.6 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 135: cis-3-(3-((4-fluoro-2-(oxetan-3-yl)-1,1-dioxido-2,3-dihydrobenzo[d]isothiazol-5-yl)amino)-1H-pyrazol-5-yl)cyclopentyl(1-methylcyclopropyl)carbamate

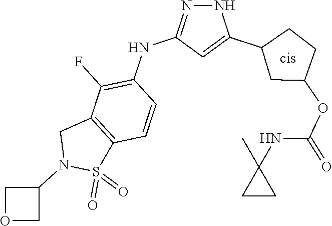 [SMILES: CC1(NC(=O)OC2CCC(c3cc(Nc4ccc5c(c4F)CN(C4COC4)S5(=O)=O)n[nH]3)C2)CC1]

The titled compound was synthesized in the procedures similar to Example 63. ¹H NMR (500 MHz, DMSO-d₆) δ 12.01 (s, 1H), 8.84 (s, 1H), 8.31 (s, 1H), 7.55 (d, J=8.7 Hz, 1H), 7.34 (s, 1H), 5.83 (s, 1H), 4.98 (s, 1H), 4.87 (t, J=6.1 Hz, 2H), 4.82-4.68 (m, 5H), 3.12-3.02 (m, 1H), 2.45-2.41 (m, 1H), 2.11-1.85 (m, 2H), 1.75-1.55 (m, 3H), 1.23 (s, 3H), 0.59 (s, 2H), 0.47 (q, J=4.6 Hz, 2H). LC-MS (ESI): m/z [M+H]⁺=506.2.

### Example 136: cis-3-(3-((4-fluoro-2-(oxetan-3-yl)-1,1-dioxido-2,3-dihydrobenzo[d]isothiazol-5-yl)amino)-1H-pyrazol-5-yl)cyclopentyl propylcarbamate

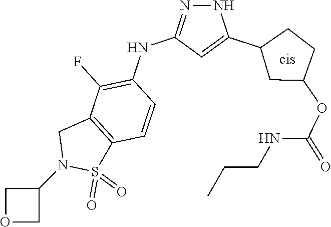 [SMILES: CCCNC(=O)OC1CCC(c2cc(Nc3ccc4c(c3F)CN(C3COC3)S4(=O)=O)n[nH]2)C1]

The titled compound was synthesized in the procedures similar to Example 63. ¹H NMR (500 MHz, DMSO-d₆)) δ 12.02 (s, 1H), 8.84 (s, 1H), 8.31 (t, J=8.0 Hz, 1H), 7.55 (d, J=8.7 Hz, 1H), 7.04 (t, J=5.4 Hz, 1H), 5.84 (s, 1H), 5.00 (d, J=4.8 Hz, 1H), 4.87 (t, J=6.1 Hz, 2H), 4.81-4.73 (m, 5H), 3.11-3.02 (m, 1H), 2.92 (dd, J=13.1, 6.6 Hz, 2H), 2.48-2.43 (m, 1H), 2.08-1.88 (m, 2H), 1.78-1.57 (m, 3H), 1.39 (dd, J=14.4, 7.2 Hz, 2H), 0.82 (t, J=7.4 Hz, 3H). LC-MS (ESI): m/z [M+H]⁺=494.2.

### Example 137:3-(3-((4-fluoro-1,1-dioxido-2,3-dihydrobenzo[d]isothiazol-5-yl)amino)-1H-pyrazol-5-yl)cyclobutyl isopropylcarbamate

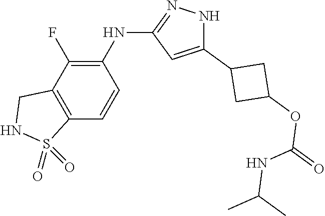 [SMILES: CC(C)NC(=O)OC1CC(c2cc(Nc3ccc4c(c3F)CNS4(=O)=O)n[nH]2)C1]

The titled compound was synthesized in the procedures similar to Example 50. ¹H NMR (500 MHz, DMSO-d₆) δ 12.07 (s, 1H), 8.74 (s, 1H), 8.25 (s, 1H), 7.69 (dd, J=10.0, 4.9 Hz, 1H), 7.48 (d, J=8.6 Hz, 1H), 7.09-7.02 (m, 1H), 5.87 (s, 1H), 4.85-4.77 (m, 1H), 4.41 (d, J=3.5 Hz, 2H), 3.64-3.56 (m, 1H), 3.12-3.05 (m, 1H), 2.72-2.64 (m, 2H), 2.12-2.05 (m, 2H), 1.04 (d, J=6.6 Hz, 6H). LC-MS (ESI): m/z [M+H]⁺=424.2.

### Example 138: cis-3-(3-((4-fluoro-1,1-dioxido-2,3-dihydrobenzo[d]isothiazol-5-yl)amino)-1H-pyrazol-5-yl)cyclopentyl propylcarbamate

### Step 1: cis-3-(3-(((benzyloxy) carbonyl)amino)-1H-pyrazol-5-yl)cyclopentyl propylcarbamate

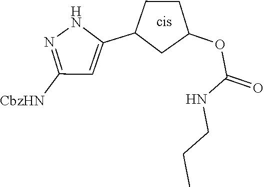 [SMILES: CCCNC(=O)OC1CCC(c2cc(NC(=O)OCc3ccccc3)n[nH]2)C1]

To a solution of cis-benzyl(5-(3-(((4-nitrophenoxy) carbonyl)oxy) cyclopentyl)-1H-pyrazol-3-yl)carbamate (7 g, 15 mmol) in 100 mL THF was added propylamine (2.6 g, 45 mmol). The resulting mixture was stirred at room temperature for 1 h. The resulting mixture was concentrated under reduced pressure, and the residue was purified by silica gel chromatography (PE/EA=2:1) to afford the product (4.6 g, 80% yield). LC-MS (ESI): m/z [M+H]⁺=387.3.

### Step 2: cis-3-(3-amino-1H-pyrazol-5-yl)cyclopentyl propylcarbamate

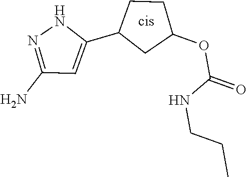 [SMILES: CCCNC(=O)OC1CCC(c2cc(N)n[nH]2)C1]

To a solution of cis-3-(3-(((benzyloxy) carbonyl)amino)-1H-pyrazol-5-yl)cyclopentyl propylcarbamate (4.6 g, 12 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with MeOH (50 mL×3). The filtrate was concentrated under reduced pressure to afford the product (2.6 g, 86% yield). LC-MS (ESI): m/z [M+H]⁺=253.4.

### Step 3: cis-3-(3-((4-fluoro-1,1-dioxido-2,3-dihydrobenzo[d]isothiazol-5-yl)amino)-1H-pyrazol-5-yl)cyclopentyl propylcarbamate

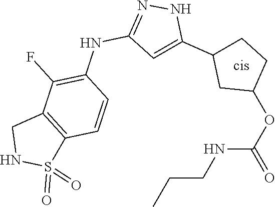 [SMILES: CCCNC(=O)OC1CCC(c2cc(Nc3ccc4c(c3F)CNS4(=O)=O)n[nH]2)C1]

A mixture of 5-bromo-4-fluoro-2,3-dihydrobenzo[d]isothiazole 1,1-dioxide (150 mg, 0.57 mmol), cis-3-(3-amino-1H-pyrazol-5-yl)cyclopentyl propylcarbamate (144.2 mg, 0.19 mmol), Brettphos Pd G3 (51.3 mg, 0.057 mmol), K₂CO₃ (235.9 mg, 1.71 mmol) in t-BuOH (20 mL) was stirred at 110° C. for 16 h under N₂ atmosphere. The mixture was concentrated under reduced pressure. The residue was purified by silica gel column chromatography, eluting with 100% EA to afford the product. The residue was purified by prep HPLC (Waters XSelect C18: RD-CO-094 column, eluting with a gradient of acetonitrile/water containing 0.1% FA, 20%-40%) to afford the product in racemic form, which was further separated by Chiral Prep-HPLC to give:

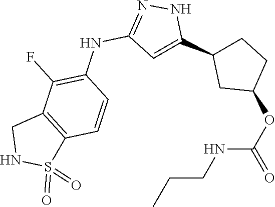 [SMILES: CCCNC(=O)O[C@@H]1CC[C@H](c2cc(Nc3ccc4c(c3F)CNS4(=O)=O)n[nH]2)C1]

Enantiomer 1 ((1R,3S)-3-(3-((4-fluoro-1,1-dioxido-2,3-dihydrobenzo[d]isothiazol-5-yl)amino)-1H-pyrazol-5-yl)cyclopentyl propylcarbamate, Example 138a, 100% ee); Retention time: 5.204 min. 1H NMR (500 MHz, DMSO-d₆)) δ 11.99 (s, 1H), 8.71 (s, 1H), 8.25 (s, 1H), 7.70 (t, J=4.9 Hz, 1H), 7.48 (d, J=8.6 Hz, 1H), 7.03 (m, 1H), 5.83 (s, 1H), 5.01 (s, 1H), 4.41 (d, J=5.2 Hz, 2H), 3.13-3.03 (m, 1H), 2.96-2.88 (m, 2H), 2.48-2.41 (m, 1H), 2.07-1.98 (m, 1H), 1.97-1.87 (m, 1H), 1.80-1.67 (m, 2H), 1.67-1.55 (m, 1H), 1.46-1.32 (m, 2H), 0.82 (t, J=7.4 Hz, 3H). LC-MS (ESI): m/z [M+H]⁺=438.2.

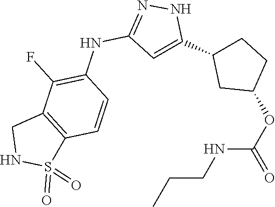 [SMILES: CCCNC(=O)O[C@H]1CC[C@@H](c2cc(Nc3ccc4c(c3F)CNS4(=O)=O)n[nH]2)C1]

Enantiomer 2 ((1S,3R)-3-(3-((4-fluoro-1,1-dioxido-2,3-dihydrobenzo[d]isothiazol-5-yl)amino)-1H-pyrazol-5-yl)cyclopentyl propylcarbamate, Example 138b, 100% ee); Retention time: 8.999 min. 1H NMR (500 MHz, DMSO-d₆)) δ 11.99 (s, 1H), 8.71 (s, 1H), 8.25 (s, 1H), 7.70 (t, J=4.9 Hz, 1H), 7.48 (d, J=8.6 Hz, 1H), 7.03 (m, 1H), 5.83 (s, 1H), 5.01 (s, 1H), 4.41 (d, J=5.2 Hz, 2H), 3.13-3.03 (m, 1H), 2.96-2.88 (m, 2H), 2.48-2.41 (m, 1H), 2.07-1.98 (m, 1H), 1.97-1.87 (m, 1H), 1.80-1.67 (m, 2H), 1.67-1.55 (m, 1H), 1.46-1.32 (m, 2H), 0.82 (t, J=7.4 Hz, 3H). LC-MS (ESI): m/z [M+H]⁺=438.2.

Chiral analytical method: Column: CHIRALPAK IE 4.6 mm×250 mm, 5 μm; Mobile phase: A for MtBE (0.1% 2M NH₃ MeOH) and B for EtOH; Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for EtOH (0.2% 2M NH₃ MeOH); Gradient: Mobile Phase A: Mobile Phase B=20: 80 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 139: cis-3-(3-((4-fluoro-1,1-dioxido-2,3-dihydrobenzo[d]isothiazol-5-yl)amino)-1H-pyrazol-5-yl)cyclopentyl((S)-sec-butyl)carbamate

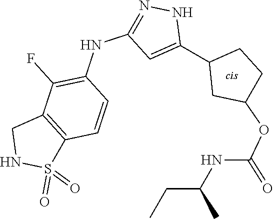 [SMILES: CC[C@H](C)NC(=O)OC1CCC(c2cc(Nc3ccc4c(c3F)CNS4(=O)=O)n[nH]2)C1]

The titled compound was synthesized in the procedures similar to Example 34 in racemic form, which was further separated by Chiral Prep-HPLC to give:

Enantiomer 1 (Example 139a, 100% ee); Retention time: 4.48 min; ¹H NMR (500 MHz, DMSO-d₆) δ 11.98 (s, 1H), 8.71 (s, 1H), 8.25 (s, 1H), 7.70 (t, J=4.8, 1H), 7.48 (d, J=8.5, 1H), 6.89 (d, J=8.2, 1H), 5.83 (s, 1H), 5.00 (s, 1H), 4.40 (d, J=4.8 Hz, 2H), 3.40 (m, 1H), 3.11-3.02 (m, 1H), 2.46 (m, 1H), 2.08-1.98 (m, 1H), 1.93 (m, 1H), 1.80-1.65 (m, 2H), 1.60 (m, 1H), 1.36 (m, 2H), 1.01 (m, 3H), 0.81 (m, 3H). LC-MS (ESI): m/z [M+H]⁺=452.2.

Enantiomer 2 (Example 139b, 100% ee); Retention time: 4.48 min; ¹H NMR (500 MHz, DMSO-d₆) δ 1.98 (s, 1H), 8.71 (s, 1H), 8.25 (s, 1H), 7.70 (t, J=4.8, 1H), 7.48 (d, J=8.5, 1H), 6.89 (d, J=8.2, 1H), 5.83 (s, 1H), 5.00 (s, 1H), 4.40 (d, J=4.8 Hz, 2H), 3.40 (m, 1H), 3.11-3.02 (m, 1H), 2.46 (m, 1H), 2.08-1.98 (m, 1H), 1.93 (m, 1H), 1.80-1.65 (m, 2H), 1.60 (m, 1H), 1.36 (m, 2H), 1.01 (m, 3H), 0.81 (m, 3H). LC-MS (ESI): m/z [M+H]⁺=452.2.

Chiral analytical method: Column: CHIRALPAK IE 4.6 mm×250 mm, 5 μm; Mobile phase: A for MtBE (0.1% 2M NH₃ MeOH) and B for EtOH; Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for EtOH (0.2% 2M NH₃ MeOH); Gradient: Mobile Phase A: Mobile Phase B=20: 80 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 140: cis-3-(3-((4-fluoro-1,1-dioxido-2,3-dihydrobenzo[d]isothiazol-5-yl)amino)-1H-pyrazol-5-yl)cyclopentyl(1-methylcyclopropyl)carbamate

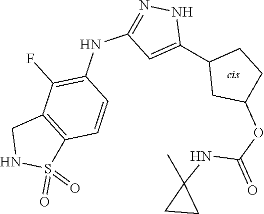 [SMILES: CC1(NC(=O)OC2CCC(c3cc(Nc4ccc5c(c4F)CNS5(=O)=O)n[nH]3)C2)CC1]

The titled compound was synthesized in the procedures similar to Example 34 in racemic form, which was further separated by Chiral Prep-HPLC to give:

Enantiomer 1 (Example 140a, 100% ee); Retention time: 4.006 min. ¹H NMR (500 MHz, DMSO-d₆) δ 11.97 (s, 1H), 8.70 (s, 1H), 8.25 (s, 1H), 7.68 (s, 1H), 7.48 (s, 1H), 7.33 (s, 1H), 5.82 (s, 1H), 4.99 (s, 1H), 4.40 (d, J=5.1 Hz, 2H), 3.10-3.04 (m, 1H), 2.47-2.44 (m, 1H), 2.10-1.85 (m, 2H), 1.75-1.53 (m, 3H), 1.23 (s, 3H), 0.59 (s, 2H), 0.47 (s, 2H). LC-MS (ESI): m/z [M+H]⁺=450.2.

Enantiomer 2 (Example 140b, 100% ee); Retention time: 5.375 min. ¹H NMR (500 MHz, DMSO-d₆) δ 11.97 (s, 1H), 8.70 (s, 1H), 8.25 (s, 1H), 7.68 (s, 1H), 7.48 (s, 1H), 7.33 (s, 1H), 5.82 (s, 1H), 4.99 (s, 1H), 4.40 (d, J=5.1 Hz, 2H), 3.10-3.04 (m, 1H), 2.47-2.44 (m, 1H), 2.10-1.85 (m, 2H), 1.75-1.53 (m, 3H), 1.23 (s, 3H), 0.59 (s, 2H), 0.47 (s, 2H). LC-MS (ESI): m/z [M+H]⁺=450.2.

Chiral analytical method: Column: CHIRALPAK IE 4.6 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 141: cis-3-(3-((4-fluoro-1,1-dioxido-2,3-dihydrobenzo[d]isothiazol-5-yl)amino)-1H-pyrazol-5-yl)cyclopentyl ethylcarbamate

### Step 1: cis-benzyl(1-(tert-butyl)-3-(3-((ethylcarbamoyl)oxy) cyclopentyl)-1H-pyrazol-5-yl)carbamate

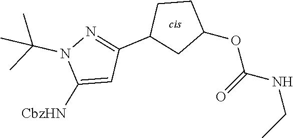 [SMILES: CCNC(=O)OC1CCC(c2cc(NC(=O)OCc3ccccc3)n(C(C)(C)C)n2)C1]

A solution of cis-benzyl(1-(tert-butyl)-3-(3-(((4-nitrophenoxy) carbonyl)oxy) cyclopentyl)-1H-pyrazol-5-yl)carbamate (1.3 g, 2.5 mmol), ethanamine (337 mg, 7.5 mmol) and DIEA (967 mg, 7.5 mmol) in THF (20 mL) was stirred for 16 h at 20° C. The solution was concentrated under reduced pressure. The residue was purified by silica gel column chromatography, eluting with PE/EtOAc (2:1) to afford the product (0.9 g, 84%), LC-MS (ESI): m/z [M+H]⁺=429.2.

### Step 2: cis-3-(5-amino-1-(tert-butyl)-1H-pyrazol-3-yl)cyclopentyl ethylcarbamate

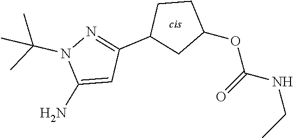 [SMILES: CCNC(=O)OC1CCC(c2cc(N)n(C(C)(C)C)n2)C1]

A mixture of cis-benzyl(1-(tert-butyl)-3-(3-((ethylcarbamoyl)oxy) cyclopentyl)-1H-pyrazol-5-yl)carbamate (0.9 g, 2.1 mmol) and Pd/C (100 mg) in THF (30 mL) was stirred for 2 h at 20° C. under H₂ atmosphere. The mixture was filtered and the filtrate was concentrated under reduced pressure to give the product (0.6 g, 95%), LC-MS (ESI): m/z [M+H]⁺=295.2.

### Step 3: cis-3-(3-((4-fluoro-1,1-dioxido-2,3-dihydrobenzo[d]isothiazol-5-yl)amino)-1H-pyrazol-5-yl)cyclopentyl ethylcarbamate

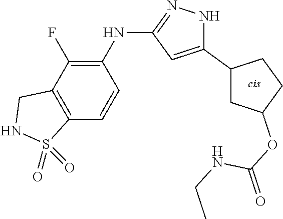 [SMILES: CCNC(=O)OC1CCC(c2cc(Nc3ccc4c(c3F)CNS4(=O)=O)n[nH]2)C1]

The titled compound was synthesized in the procedures similar to Example 8, step 5 to step 6. ¹H NMR (500 MHz, DMSO-d₆) δ 12.00 (s, 1H), 8.71 (s, 1H), 8.24 (s, 1H), 7.70 (s, 1H), 7.48 (d, J=8.6 Hz, 1H), 7.03 (s, 1H), 5.83 (s, 1H), 5.10-4.92 (m, 1H), 4.41 (s, 2H), 3.12-3.03 (m, 1H), 3.02-2.93 (m, 2H), 2.59-2.56 (m, 1H), 2.06-1.98 (m, 1H), 1.96-1.85 (m, 1H), 1.79-1.54 (m, 3H), 1.00 (t, J=7.2 Hz, 3H). LC-MS (ESI): m/z [M+H]⁺=424.20.

### Example 142 and 143: cis-3-(3-((4-fluoro-1,1-dioxido-2,3-dihydrobenzo[d]isothiazol-5-yl)amino)-1H-pyrazol-5-yl)cyclopentyl(4-hydroxycyclohexyl)carbamate

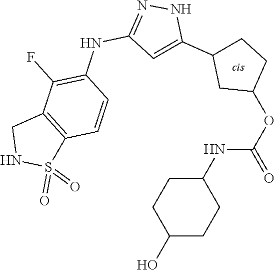 [SMILES: O=C(NC1CCC(O)CC1)OC1CCC(c2cc(Nc3ccc4c(c3F)CNS4(=O)=O)n[nH]2)C1]

The two titled compound (obtained by Prep-HPLC) was synthesized in the procedures similar to

### Example 34

Diastereoisomer 1 (Example 142): ¹H NMR (500 MHz, DMSO-d₆) δ 11.97 (s, 1H), 8.69 (s, 1H), 8.25 (t, J=5 Hz, 1H), 7.68 (t, J=5 Hz, 1H), 7.47 (d, J=10 Hz, 1H), 6.92 (d, J=10 Hz, 1H), 5.82 (s, 1H), 5.03-4.95 (m, 1H), 4.51-4.45 (m, 1H), 4.39 (d, J=5 Hz, 2H), 3.22-3.14 (m, 1H), 3.10-3.01 (m, 1H), 2.48-2.40 (m, 2H), 2.06-1.99 (m, 1H), 1.95-1.86 (m, 1H), 1.81-1.65 (m, 6H), 1.63-1.54 (m, 1H), 1.20-1.10 (m, 4H). LC-MS (ESI): m/z [M+H]⁺=494.2.

Diastereoisomer 2 (Example 143): ¹H NMR (500 MHz, DMSO-d₆) δ 11.97 (s, 1H), 8.69 (s, 1H), 8.30-8.21 (m 1H), 7.68 (t, J=5 Hz, 1H), 7.47 (d, J=10 Hz, 1H), 6.96 (d, J=10 Hz, 1H), 5.83 (s, 1H), 5.03-4.96 (m, 1H), 4.43-4.38 (m, 2H), 4.31-4.27 (m, 1H), 3.70-3.63 (m, 1H), 3.29-3.25 (m, 1H), 3.12-3.01 (m, 1H), 2.48-2.42 (m, 1H), 2.06-2.98 (m, 1H), 1.95-1.85 (m, 1H), 1.79-1.65 (m, 2H), 1.65-1.50 (m, 5H), 148-1.33 (m, 4H). LC-MS (ESI): m/z [M+H]⁺=494.2.

### Example 144: cis-3-(3-((4-fluoro-1,1-dioxido-2,3-dihydrobenzo[d]isothiazol-5-yl)amino)-1H-pyrazol-5-yl)cyclopentyl((1s,4s)-4-hydroxy-4-methylcyclohexyl)carbamate

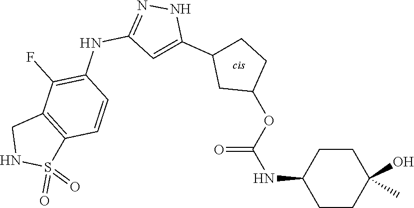 [SMILES: C[C@]1(O)CC[C@@H](NC(=O)OC2CCC(c3cc(Nc4ccc5c(c4F)CNS5(=O)=O)n[nH]3)C2)CC1]

The titled compound was synthesized in the procedures similar to Example 34. ¹H NMR (500 MHz, DMSO-d₆) δ 11.97 (s, 1H), 8.71 (s, 1H), 8.29-8.22 (m 1H), 7.69 (t, J=5 Hz, 1H), 7.47 (d, J=10 Hz, 1H), 6.94 (d, J=10 Hz, 1H), 5.83 (s, 1H), 5.02-4.95 (m, 1H), 4.42-4.36 (m, 2H), 3.95 (s, 1H), 3.22-3.11 (m, 1H), 3.10-3.01 (m, 1H), 2.48-2.36 (m, 2H), 2.05-1.97 (m, 1H), 1.94-1.83 (m, 1H), 1.78-1.64 (m, 2H), 1.63-1.36 (m, 6H), 1.32-1.20 (m, 2H), 1.07 (s, 3H). LC-MS (ESI): m/z [M+H]⁺=508.2.

### Example 145: cis-3-(3-((4-fluoro-1,1-dioxido-2,3-dihydrobenzo[d]isothiazol-5-yl)amino)-1H-pyrazol-5-yl)cyclopentyl(2S,3R)-3-hydroxy-2-methylazetidine-1-carboxylate

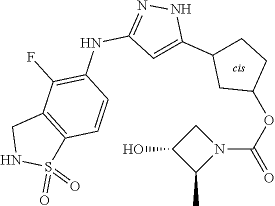 [SMILES: C[C@H]1[C@H](O)CN1C(=O)OC1CCC(c2cc(Nc3ccc4c(c3F)CNS4(=O)=O)n[nH]2)C1]

The titled compound was synthesized in the procedures similar to Example 34. ¹H NMR (500 MHz, DMSO-d₆) δ 12.00 (s, 1H), 8.71 (s, 1H), 8.26 (s, 1H), 7.68 (t, J=4.9 Hz, 1H), 7.47 (d, J=8.5 Hz, 1H), 5.82 (s, 1H), 5.61 (d, J=6.0 Hz, 1H), 5.02 (s, 1H), 4.40 (d, J=5.1 Hz, 2H), 3.97-3.84 (m, 3H), 3.47 (s, 1H), 3.14-3.05 (m, 1H), 2.48-2.43 (m, 1H), 2.06-1.84 (m, 2H), 1.78-1.61 (m, 3H), 1.29-1.21 (m, 3H). LC-MS (ESI): m/z [M+H]⁺=466.2.

### Example 146: cis-3-(3-((4-fluoro-1,1-dioxido-2,3-dihydrobenzo[d]isothiazol-5-yl)amino)-1H-pyrazol-5-yl)cyclopentyl(2-hydroxy-2-methylpropyl)carbamate

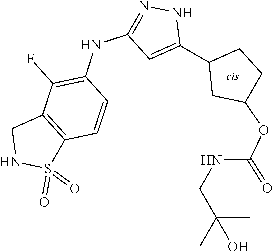 [SMILES: CC(C)(O)CNC(=O)OC1CCC(c2cc(Nc3ccc4c(c3F)CNS4(=O)=O)n[nH]2)C1]

The titled compound was synthesized in the procedures similar to Example 34. ¹H NMR (500 MHz, DMSO-d₆) δ 11.97 (s, 1H), 8.69 (s, 1H), 8.30-8.19 (m 1H), 7.68 (t, J=5 Hz, 1H), 7.47 (d, J=10 Hz, 1H), 6.84 (d, J=5 Hz, 1H), 5.84 (s, 1H), 5.05-4.97 (m, 1H), 4.40 (d, J=5 Hz, 2H), 4.33 (s, 1H), 3.13-3.02 (m, 1H), 2.97-2.83 (m, 2H), 2.48-2.43 (m, 1H), 2.06-1.99 (m, 1H), 1.96-1.85 (m, 1H), 1.80-1.67 (m, 2H), 1.66-1.57 (m, 1H), 1.03 (s, 6H). LC-MS (ESI): m/z [M+H]⁺=468.2.

### Example 147: cis-3-(3-((4-fluoro-1,1-dioxido-2,3-dihydrobenzo[d]isothiazol-5-yl)amino)-1H-pyrazol-5-yl)cyclopentyl(1-(oxetan-3-yl)ethyl)carbamate

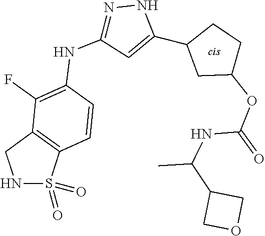 [SMILES: CC(NC(=O)OC1CCC(c2cc(Nc3ccc4c(c3F)CNS4(=O)=O)n[nH]2)C1)C1COC1]

The titled compound was synthesized in the procedures similar to Example 117. ¹H NMR (500 MHz, DMSO-d₆) δ=11.99 (s, 1H), 8.70 (d, J=10.8, 1H), 8.26 (s, 1H), 7.69 (s, 1H), 7.48 (d, J=8.6, 1H), 7.13-7.02 (m, 1H), 5.82 (s, 1H), 5.01 (s, 1H), 4.53 (m, 2H), 4.40 (d, J=4.0, 2H), 4.35 (d, J=5.9, 1H), 4.25 (t, J=6.1, 1H), 3.86-3.76 (m, 1H), 3.07 (m, 1H), 2.90 (m, 1H), 2.47-2.43 (m, 1H), 2.05 (m, 1H), 1.98-1.85 (m, 1H), 1.72 (m, 2H), 1.61 (m, 1H), 0.95 (m, 3H). LC-MS (ESI): m/z [M+H]⁺=480.3.

### Example 148: cis-3-(3-((4-fluoro-1,1-dioxido-2,3-dihydrobenzo[d]isothiazol-5-yl)amino)-1H-pyrazol-5-yl)cyclopentyl(oxetan-3-ylmethyl)carbamate

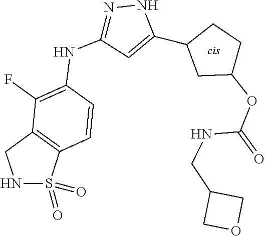 [SMILES: O=C(NCC1COC1)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.99 (s, 1H), 8.70 (s, 1H), 8.26 (s, 1H), 7.69 (s, 1H), 7.48 (d, J=8.6, 1H), 7.26 (t, J=5.6, 1H), 5.82 (s, 1H), 5.01 (s, 1H), 4.57 (m, 2H), 4.40 (s, 2H), 4.25 (t, J=5.7, 2H), 3.24 (t, J=6.3, 2H), 3.07 (m, 1H), 3.00 (m, 1H), 2.48-2.42 (m, 1H), 2.06-1.99 (m, 1H), 1.96-1.85 (m, 1H), 1.71 (m, 2H), 1.65-1.55 (m, 1H). LC-MS (ESI): m/z [M+H]⁺=466.29.

### Example 149: cis-3-(3-((4-fluoro-1,1-dioxido-2,3-dihydrobenzo[d]isothiazol-5-yl)amino)-1H-pyrazol-5-yl)cyclopentyl oxetan-3-ylcarbamate

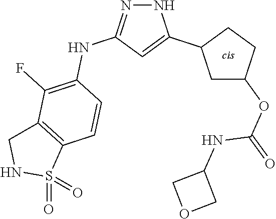 [SMILES: O=C(NC1COC1)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.99 (s, 1H), 8.71 (s, 1H), 8.26 (s, 1H), 7.90 (d, J=5.2, 1H), 7.69 (t, J=4.9, 1H), 7.48 (d, J=8.6, 1H), 5.83 (s, 1H), 5.00 (m, 1H), 4.64 (s, 3H), 4.41 (m, 4H), 3.16-2.94 (m, 1H), 2.48-2.42 (m, 1H), 2.06-1.98 (m, 1H), 1.91 (m, 1H), 1.74 (m, 2H), 1.62 (m, 1H). LC-MS (ESI): m/z [M+H]⁺=452.31.

### Example 150: cis-3-(3-((4-fluoro-1,1-dioxido-2,3-dihydrobenzo[d]isothiazol-5-yl)amino)-1H-pyrazol-5-yl)cyclopentyl bicyclo[1.1.1]pentan-1-ylcarbamate

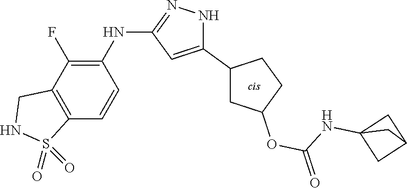 [SMILES: O=C(NC12CC(C1)C2)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.98 (s, 1H), 8.72 (s, 1H), 8.26 (t, J=7.6, 1H), 7.74 (m, 1H), 7.68 (m, 1H), 7.47 (d, J=8.6, 1H), 5.83 (s, 1H), 5.00 (s, 1H), 4.40 (d, J=3.3, 2H), 3.11-3.00 (m, 1H), 2.47-2.42 (m, 1H), 2.38-2.28 (m, 1H), 2.03 (m, 1H), 1.90 (m, 7H), 1.71 (m, 2H), 1.59 (m, 1H). LC-MS (ESI): m/z [M+H]⁺=462.3.

### Example 151: cis-3-(3-((4-fluoro-1,1-dioxido-2,3-dihydrobenzo[d]isothiazol-5-yl)amino)-1H-pyrazol-5-yl)cyclopentyl spiro[3.3]heptan-2-ylcarbamate

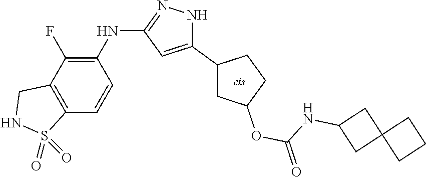 [SMILES: O=C(NC1CC2(CCC2)C1)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.98 (s, 1H), 8.71 (d, J=1.7, 1H), 8.24 (s, 1H), 7.69 (t, J=5.1, 1H), 7.47 (d, J=8.6, 1H), 7.28 (d, J=7.9, 1H), 5.82 (s, 1H), 4.98 (m, 1H), 4.40 (d, J=5.0, 2H), 3.78 (m, 1H), 3.11-2.98 (m, 1H), 2.45 (m, 1H), 2.21 (m, 2H), 2.05-1.90 (m, 3H), 1.90-1.63 (m, 9H), 1.62-1.53 (m, 1H). LC-MS (ESI): m/z [M+H]⁺=490.29.

### Example 152: cis-3-(3-((4-fluoro-1,1-dioxido-2,3-dihydrobenzo[d]isothiazol-5-yl)amino)-1H-pyrazol-5-yl)cyclopentyl spiro[2.3]hexan-4-ylcarbamate

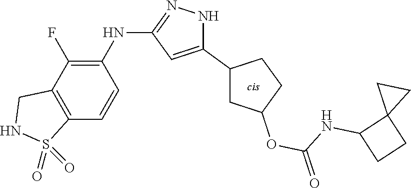 [SMILES: O=C(NC1CCC12CC2)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.98 (s, 1H), 8.71 (s, 1H), 8.25 (s, 1H), 7.69 (t, J=5.0, 1H), 7.48 (d, J=8.6, 1H), 7.23 (d, J=8.2, 1H), 5.83 (s, 1H), 4.95 (m, 1H), 4.41 (m, 2H), 4.19-4.09 (m, 1H), 3.05 (m, 1H), 2.48-2.39 (m, 1H), 2.22 (m, 1H), 1.99 (m, 3H), 1.89 (s, 1H), 1.75-1.64 (m, 3H), 1.57 (m, 1H), 0.53 (m, 1H), 0.45-0.30 (m, 2H), 0.30-0.18 (m, 1H). LC-MS (ESI): m/z [M+H]⁺=476.35.

### Example 153: cis-3-(3-((4-fluoro-1,1-dioxido-2,3-dihydrobenzo[d]isothiazol-5-yl)amino)-1H-pyrazol-5-yl)cyclopentyl methylcarbamate

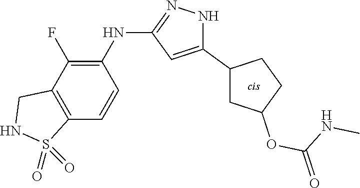 [SMILES: CNC(=O)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.98 (s, 1H), 8.71 (s, 1H), 8.26 (t, J=8.1, 1H), 7.69 (s, 1H), 7.48 (d, J=8.6, 1H), 6.91 (d, J=4.2, 1H), 5.83 (s, 1H), 5.04-4.92 (m, 1H), 4.40 (d, J=3.9, 2H), 3.07 (m, 1H), 2.55 (d, J=4.6, 3H), 2.45 (m, 1H), 2.06-1.98 (m, 1H), 1.95-1.85 (m, 1H), 1.77-1.65 (m, 2H), 1.65-1.56 (m, 1H).

LC-MS (ESI): m/z [M+H]⁺=410.31.

### Example 154: cis-3-(3-((4-fluoro-1,1-dioxido-2,3-dihydrobenzo[d]isothiazol-5-yl)amino)-1H-pyrazol-5-yl)cyclopentyl(3,3,3-trifluoropropyl)carbamate

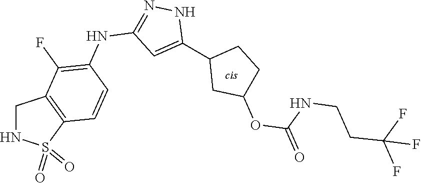 [SMILES: O=C(NCCC(F)(F)F)OC1CCC(c2cc(Nc3ccc4c(c3F)CNS4(=O)=O)n[nH]2)C1]

The titled compound was synthesized in the procedures similar to Example 117 in a racemic form, which was further separated by Chiral Prep-HPLC to give:

Enantiomer 1 (Example 154a, 100% ee); Retention time: 4.649 min. ¹H NMR (500 MHz, DMSO-d₆) δ=11.99 (s, 1H), 8.71 (s, 1H), 8.26 (s, 1H), 7.69 (s, 1H), 7.48 (d, J=8.5, 1H), 7.26 (t, J=5.6, 1H), 5.83 (s, 1H), 5.03 (s, 1H), 4.40 (d, J=4.5, 2H), 3.21 (m, 2H), 3.11-3.01 (m, 1H), 2.47 (s, 1H), 2.45-2.35 (m, 2H), 2.04 (m, 1H), 1.98-1.85 (m, 1H), 1.80-1.66 (m, 2H), 1.62 (m, 1H). LC-MS (ESI): m/z [M+H]⁺=492.22.

Enantiomer 1 (Example 154b, 100% ee); Retention time: 6.746 min. ¹H NMR (500 MHz, DMSO-d₆) δ=11.99 (s, 1H), 8.71 (s, 1H), 8.26 (s, 1H), 7.69 (s, 1H), 7.48 (d, J=8.5, 1H), 7.26 (t, J=5.6, 1H), 5.83 (s, 1H), 5.03 (s, 1H), 4.40 (d, J=4.5, 2H), 3.21 (m, 2H), 3.11-3.01 (m, 1H), 2.47 (s, 1H), 2.45-2.35 (m, 2H), 2.04 (m, 1H), 1.98-1.85 (m, 1H), 1.80-1.66 (m, 2H), 1.62 (m, 1H). LC-MS (ESI): m/z [M+H]⁺=492.22.

Chiral analytical method: Column: CHIRALPAK IE 4.6 mm×250 mm, 5 μm; Mobile phase: A for MTBE (0.1% 2M NH₃ MeOH) and B for MeOH/DCM (1/1); Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MeOH/DCM (1/1); Gradient: Mobile Phase A: Mobile Phase

B=10: 90 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 155: cis-3-(3-((4-fluoro-1,1-dioxido-2,3-dihydrobenzo[d]isothiazol-5-yl)amino)-1H-pyrazol-5-yl)cyclopentyl 2,2-dimethylazetidine-1-carboxylate

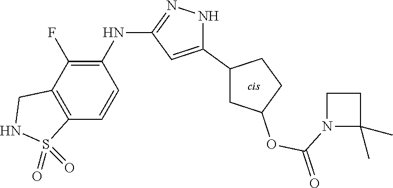 [SMILES: CC1(C)CCN1C(=O)OC1CCC(c2cc(Nc3ccc4c(c3F)CNS4(=O)=O)n[nH]2)C1]

The titled compound was synthesized in the procedures similar to Example 117. LC-MS (ESI): m/z [M+H]⁺=464.34; 1H NMR (500 MHz, DMSO-d₆) δ=12.02 (s, 1H), 8.73 (s, 1H), 8.26 (s, 1H), 7.69 (t, J=4.9, 1H), 7.47 (d, J=8.6, 1H), 5.82 (s, 1H), 5.01 (m, 1H), 4.40 (d, J=5.0, 2H), 3.72 (t, J=7.7, 1H), 3.65 (t, J=7.6, 1H), 3.13-3.03 (m, 1H), 2.47-2.39 (m, 1H), 2.08-1.98 (m, 1H), 1.98-1.91 (m, 2H), 1.91-1.82 (m, 1H), 1.74 (m, 2H), 1.65 (m, 1H), 1.42-1.27 (m, 6H).

### Example 156: cis-3-(3-((4-fluoro-1,1-dioxido-2,3-dihydrobenzo[d]isothiazol-5-yl)amino)-1H-pyrazol-5-yl)cyclopentyl(1-cyclopropylethyl)carbamate

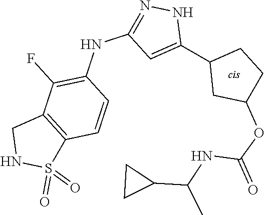 [SMILES: CC(NC(=O)OC1CCC(c2cc(Nc3ccc4c(c3F)CNS4(=O)=O)n[nH]2)C1)C1CC1]

The titled compound was synthesized in the procedures similar to Example 117. ¹H NMR (500 MHz, DMSO-d₆) δ=11.99 (s, 1H), 8.71 (s, 1H), 8.25 (s, 1H), 7.69 (t, J=5.1, 1H), 7.48 (d, J=8.6, 1H), 7.00 (d, J=8.0, 1H), 5.83 (s, 1H), 4.99 (s, 1H), 4.41 (d, J=5.1, 2H), 3.06 (m, 1H), 2.99 (m, 1H), 2.46 (m, 1H), 2.03 (m, 1H), 1.97-1.85 (m, 1H), 1.73 (m, 2H), 1.60 (m, 1H), 1.08 (m, 3H), 0.81 (m, 1H), 0.41-0.33 (m, 1H), 0.33-0.27 (m, 1H), 0.23 (m, 1H), 0.09 (m, 1H). LC-MS (ESI): m/z [M+H]⁺=464.32.

### Example 157: cis-3-(3-((4-fluoro-1,1-dioxido-2,3-dihydrobenzo[d]isothiazol-5-yl)amino)-1H-pyrazol-5-yl)cyclopentyl cyclobutylcarbamate

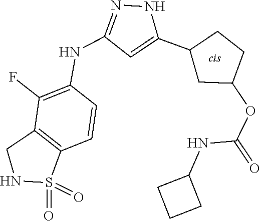 [SMILES: O=C(NC1CCC1)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.99 (s, 1H), 8.71 (s, 1H), 8.25 (s, 1H), 7.69 (t, J=5.1, 1H), 7.48 (d, J=8.6, 1H), 7.36 (d, J=7.8, 1H), 5.83 (s, 1H), 4.99 (s, 1H), 4.40 (d, J=5.1, 2H), 3.94 (m, 1H), 3.17-3.00 (m, 1H), 2.47-2.39 (m, 1H), 2.09 (m, 2H), 2.02 (m, 1H), 1.88 (m, 3H), 1.70 (m, 2H), 1.64-1.46 (m, 3H). LC-MS (ESI): m/z [M+H]⁺=450.31.

### Example 158: cis-3-(3-((4-fluoro-1,1-dioxido-2,3-dihydrobenzo[d]isothiazol-5-yl)amino)-1H-pyrazol-5-yl)cyclopentyl(1-(methoxymethyl)cyclopropyl)carbamate

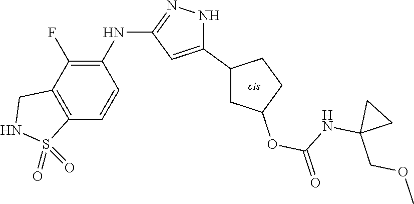 [SMILES: COCC1(NC(=O)OC2CCC(c3cc(Nc4ccc5c(c4F)CNS5(=O)=O)n[nH]3)C2)CC1]

The titled compound was synthesized in the procedures similar to Example 117 in a racemic form, which was further purified by Chiral Prep-HPLC to give:

Enantiomer 1 (Example 158a, 99.96% ee); Retention time: 4.499 min. ¹H NMR (500 MHz, DMSO-d₆) δ=11.97 (s, 1H), 8.70 (s, 1H), 8.26 (t, J=7.6, 1H), 7.69 (s, 1H), 7.47 (d, J=8.6, 1H), 7.40 (s, 1H), 5.83 (s, 1H), 4.99 (s, 1H), 4.41 (s, 2H), 3.23 (s, 3H), 3.07 (m, 1H), 2.47-2.42 (m, 1H), 2.02 (d, m, 1H), 1.90 (m, 1H), 1.70 (m, 2H), 1.58 (m, 1H), 0.62 (s, 4H). LC-MS (ESI): m/z [M+H]⁺=480.26.

Enantiomer 2 (Example 158b, 99.96% ee); Retention time: 6.026 min. ¹H NMR (500 MHz, DMSO-d₆) δ=11.97 (s, 1H), 8.70 (s, 1H), 8.26 (t, J=7.6, 1H), 7.69 (s, 1H), 7.47 (d, J=8.6, 1H), 7.40 (s, 1H), 5.83 (s, 1H), 4.99 (s, 1H), 4.41 (s, 2H), 3.23 (s, 3H), 3.07 (m, 1H), 2.47-2.42 (m, 1H), 2.02 (d, m, 1H), 1.90 (m, 1H), 1.70 (m, 2H), 1.58 (m, 1H), 0.62 (s, 4H). LC-MS (ESI): m/z [M+H]⁺=480.26.

Chiral analytical method: Column: CHIRALPAK IE 4.6 mm×250 mm, 5 μm; Mobile phase: A for MTBE (0.1% 2M NH₃ MeOH) and B for MeOH/DCM (1/1); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MeOH/DCM (1/1); Gradient: Mobile Phase A: Mobile Phase B=20: 80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 159: cis-3-(3-((4-fluoro-1,1-dioxido-2,3-dihydrobenzo[d]isothiazol-5-yl)amino)-1H-pyrazol-5-yl)cyclopentyl tert-butylcarbamate

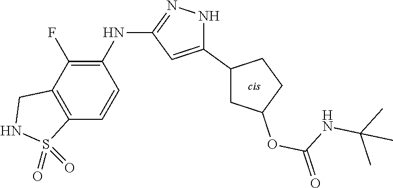 [SMILES: CC(C)(C)NC(=O)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.97 (s, 1H), 8.72 (s, 1H), 8.26 (s, 1H), 7.69 (t, J=5.1, 1H), 7.48 (d, J=8.6, 1H), 6.76 (s, 1H), 5.83 (s, 1H), 4.97 (s, 1H), 4.41 (d, J=5.2, 2H), 3.11-2.99 (m, 1H), 2.48-2.42 (m, 1H), 2.02 (m, 1H), 1.97-1.84 (m, 1H), 1.78-1.64 (m, 2H), 1.58 (m, 1H), 1.21 (s, 9H). LC-MS (ESI): m/z [M+H]⁺=452.36.

### Example 160: cis-3-(3-((4-fluoro-1,1-dioxido-2,3-dihydrobenzo[d]isothiazol-5-yl)amino)-1H-pyrazol-5-yl)cyclopentyl spiro[2.3]hexan-5-ylcarbamate

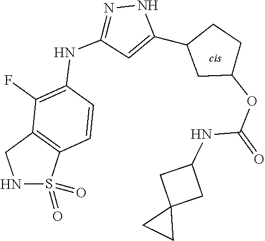 [SMILES: O=C(NC1CC2(CC2)C1)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.98 (s, 1H), 8.71 (s, 1H), 8.26 (s, 1H), 7.69 (t, J=5.1, 1H), 7.48 (d, J=8.6, 1H), 7.45 (s, 1H), 5.83 (s, 1H), 4.99 (s, 1H), 4.40 (d, J=5.2, 2H), 4.14 (m, 1H), 3.14-2.99 (m, 1H), 2.47-2.40 (m, 1H), 2.19 (m, 2H), 2.14-2.07 (m, 2H), 2.03 (m, 1H), 1.92 (m, 1H), 1.71 (m, 2H), 1.60 (m, 1H), 0.45-0.38 (m, 2H), 0.38-0.28 (m, 2H). LC-MS (ESI): m/z [M+H]⁺=476.29.

### Example 161: cis-3-(3-((4-fluoro-1,1-dioxido-2,3-dihydrobenzo[d]isothiazol-5-yl)amino)-1H-pyrazol-5-yl)cyclopentyl(1-methoxypropan-2-yl)carbamate

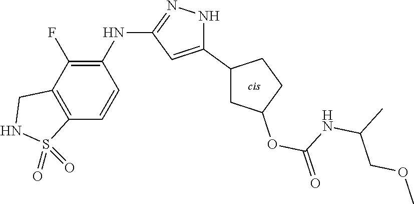 [SMILES: COCC(C)NC(=O)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.99 (s, 1H), 8.71 (s, 1H), 8.24 (s, 1H), 7.69 (s, 1H), 7.48 (d, J=8.6, 1H), 6.95 (d, J=8.1, 1H), 5.83 (s, 1H), 5.00 (s, 1H), 4.40 (s, 2H), 3.67-3.60 (m, 1H), 3.22 (m, 4H), 3.15-3.10 (m, 1H), 3.10-3.00 (m, 1H), 2.46 (m, 1H), 2.08-1.99 (m, 1H), 1.97-1.84 (m, 1H), 1.71 (m, 2H), 1.60 (m, 1H), 1.05-0.95 (m, 3H). LC-MS (ESI): m/z [M+H]⁺=468.34.

### Example 162: cis-3-(3-((4-fluoro-1,1-dioxido-2,3-dihydrobenzo[d]isothiazol-5-yl)amino)-1H-pyrazol-5-yl)cyclopentyl(3-methyltetrahydrofuran-3-yl)carbamate

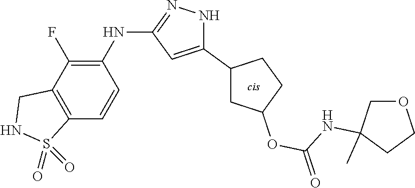 [SMILES: CC1(NC(=O)OC2CCC(c3cc(Nc4ccc5c(c4F)CNS5(=O)=O)n[nH]3)C2)CCOC1]

The titled compound was synthesized in the procedures similar to Example 117 in racemic form, which was further purified by Chiral Prep-HPLC to obtain:

Enantiomer 1 (Example 162a, 100% ee); Retention time: 4.792 min. ¹H NMR (500 MHz, DMSO-d₆) δ=11.99 (s, 1H), 8.72 (s, 1H), 8.26 (t, J=8.0, 1H), 7.69 (m, 1H), 7.48 (d, J=8.5, 1H), 7.23 (s, 1H), 5.83 (d, J=1.9, 1H), 5.00 (m, 1H), 4.41 (d, J=3.0, 2H), 3.82 (d, J=6.9, 1H), 3.73 (t, J=7.1, 2H), 3.43 (d, J=8.6, 1H), 3.12-3.01 (m, 1H), 2.46 (m, 1H), 2.16 (m, 1H), 2.06-1.99 (m, 1H), 1.98-1.85 (m, 1H), 1.79-1.65 (m, 3H), 1.60 (m, 1H), 1.32 (s, 3H). LC-MS (ESI): m/z [M+H]⁺=480.28.

Enantiomer 2 (Example 162b, 100% ee); Retention time: 8.768 min. ¹H NMR (500 MHz, DMSO-d₆) δ=11.99 (s, 1H), 8.71 (s, 1H), 8.26 (t, J=8.0, 1H), 7.69 (m, 1H), 7.48 (d, J=8.6, 1H), 7.23 (s, 1H), 5.83 (d, J=1.9, 1H), 5.00 (m, 1H), 4.41 (s, 2H), 3.82 (d, J=7.5, 1H), 3.73 (t, J=7.1, 2H), 3.43 (d, J=8.6, 1H), 3.12-3.00 (m, 1H), 2.46 (m, 1H), 2.16 (m, 1H), 2.03 (m, 1H), 1.96-1.86 (m, 1H), 1.82-1.66 (m, 3H), 1.60 (m, 1H), 1.32 (s, 3H). LC-MS (ESI): m/z [M+H]⁺=480.31.

Enantiomer 3 (Example 162c, 100% ee); Retention time: 9.447 min. ¹H NMR (500 MHz, DMSO-d₆) δ=11.99 (s, 1H), 8.71 (s, 1H), 8.26 (t, J=7.9, 1H), 7.69 (m, 1H), 7.48 (d, J=8.6, 1H), 7.23 (s, 1H), 5.83 (s, 1H), 5.00 (m, 1H), 4.41 (d, J=4.9, 2H), 3.82 (d, J=7.8, 1H), 3.73 (t, J=7.1, 2H), 3.43 (d, J=8.6, 1H), 3.11-3.02 (m, 1H), 2.49-2.45 (m, 1H), 2.16 (m, 1H), 2.03 (m, 1H), 1.93 (m, 1H), 1.79-1.65 (m, 3H), 1.60 (m, 1H), 1.32 (s, 3H). LC-MS (ESI): m/z [M+H]⁺=480.31.

Enantiomer 4 (Example 162d, 100% ee); Retention time: 11.522 min. ¹H NMR (500 MHz, DMSO-d₆) δ=11.99 (s, 1H), 8.71 (s, 1H), 8.26 (t, J=8.0, 1H), 7.69 (s, 1H), 7.48 (d, J=8.6, 1H), 7.23 (s, 1H), 5.83 (d, J=1.9, 1H), 5.00 (m, 1H), 4.41 (s, 2H), 3.82 (d, J=7.8, 1H), 3.73 (t, J=7.1, 2H), 3.43 (d, J=8.6, 1H), 3.12-3.00 (m, 1H), 2.46 (m, 1H), 2.16 (m, 1H), 2.07-1.99 (m, 1H), 1.96-1.85 (m, 1H), 1.79-1.65 (m, 3H), 1.60 (m, 1H), 1.32 (s, 3H). LC-MS (ESI): m/z [M+H]⁺=480.28.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=20: 80 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=20: 80 (v/v); Flow Rate: 18 mL/min, Wavelength: UV 280 nm and 300 nm, Prep-HPLC Equipment: Prep-HPLC-Gilson; Column temperature: 25° C.

### Example 163: cis-3-(3-((4-fluoro-1,1-dioxido-2,3-dihydrobenzo[d]isothiazol-5-yl)amino)-1H-pyrazol-5-yl)cyclopentyl(1-methylcyclobutyl)carbamate

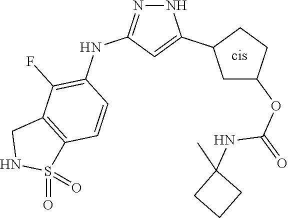 [SMILES: CC1(NC(=O)OC2CCC(c3cc(Nc4ccc5c(c4F)CNS5(=O)=O)n[nH]3)C2)CCC1]

The titled compound was synthesized in the procedures similar to Example 117. ¹H NMR (500 MHz, DMSO-d₆) δ=11.98 (s, 1H), 8.72 (s, 1H), 8.25 (s, 1H), 7.69 (t, J=5.1, 1H), 7.48 (d, J=8.6, 1H), 7.16 (s, 1H), 5.83 (s, 1H), 4.99 (s, 1H), 4.41 (d, J=5.2, 2H), 3.17-2.92 (m, 1H), 2.48-2.43 (m, 1H), 2.22 (m, 2H), 2.06-1.98 (m, 1H), 1.92 (m, 1H), 1.80 (m, 2H), 1.77-1.64 (m, 4H), 1.60 (m, 1H), 1.31 (s, 3H). LC-MS (ESI): m/z [M+H]⁺=464.36.

### Example 164: cis-3-(3-((4-fluoro-1,1-dioxido-2,3-dihydrobenzo[d]isothiazol-5-yl)amino)-1H-pyrazol-5-yl)cyclopentyl(3-methyloxetan-3-yl)carbamate

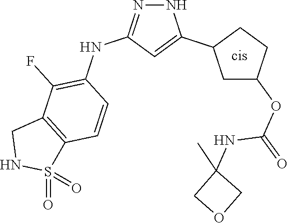 [SMILES: CC1(NC(=O)OC2CCC(c3cc(Nc4ccc5c(c4F)CNS5(=O)=O)n[nH]3)C2)COC1]

The titled compound was synthesized in the procedures similar to Example 117 in a racemic form, which was further separated by Chiral Prep-HPLC to give:

Enantiomer 1 (100% ee); Retention time: 4.851 min. ¹H NMR (500 MHz, DMSO-d₆) δ=12.00 (s, 1H), 8.72 (s, 1H), 8.27 (t, J=7.7, 1H), 7.69 (t, J=5.0, 1H), 7.62 (s, 1H), 7.48 (d, J=8.6, 1H), 5.83 (s, 1H), 5.02 (m, 1H), 4.56 (s, 2H), 4.41 (d, J=5.1, 2H), 4.24 (d, J=4.9, 2H), 3.14-3.01 (m, 1H), 2.46 (m, 1H), 2.04 (m, 1H), 1.91 (m, 1H), 1.73 (m, 2H), 1.66-1.57 (m, 1H), 1.47 (s, 3H). LC-MS (ESI): m/z [M+H]⁺=466.29.

Enantiomer 2 (100% ee); Retention time: 7.096 min. ¹H NMR (500 MHz, DMSO-d₆) δ=12.00 (s, 1H), 8.72 (s, 1H), 8.27 (t, J=8.0, 1H), 7.69 (s, 1H), 7.62 (s, 1H), 7.48 (d, J=8.6, 1H), 5.83 (d, J=1.8, 1H), 5.02 (m, 1H), 4.56 (s, 2H), 4.41 (t, J=5.8, 2H), 4.24 (d, J=4.5, 2H), 3.14-2.98 (m, 1H), 2.46 (m, 1H), 2.04 (m, 1H), 1.91 (m, 1H), 1.73 (m, 2H), 1.66-1.57 (m, 1H), 1.47 (s, 3H). LC-MS (ESI): m/z [M+H]⁺=466.28.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=20: 80 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=20: 80 (v/v); Flow Rate: 18 mL/min, Wavelength: UV 280 nm and 300 nm, Prep-HPLC Equipment: Prep-HPLC-Gilson; Column temperature: 25° C.

### Example 165: cis-3-(3-((4-fluoro-1,1-dioxido-2,3-dihydrobenzo[d]isothiazol-5-yl)amino)-1H-pyrazol-5-yl)cyclopentyl(4,4,4-trifluorobutan-2-yl)carbamate

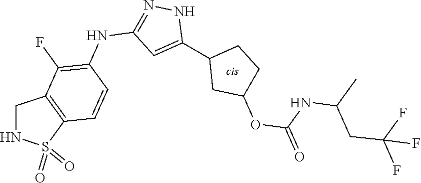 [SMILES: CC(CC(F)(F)F)NC(=O)OC1CCC(c2cc(Nc3ccc4c(c3F)CNS4(=O)=O)n[nH]2)C1]

The titled compound was synthesized in the procedures similar to Example 117. ¹H NMR (500 MHz, DMSO-d₆) δ=11.99 (s, 1H), 8.71 (s, 1H), 8.25 (s, 1H), 7.69 (d, J=5.0, 1H), 7.47 (d, J=8.5, 1H), 5.82 (s, 1H), 5.01 (s, 1H), 4.40 (d, J=5.2, 2H), 3.83 (m, 1H), 3.07 (m, 1H), 2.47-2.44 (m, 1H), 2.43-2.28 (m, 2H), 2.03 (m, 1H), 1.91 (s, 1H), 1.70 (m, 2H), 1.60 (m, 1H), 1.16-1.08 (m, 3H). LC-MS (ESI): m/z [M+H]⁺=506.28.

### Example 166 and 167: cis-3-(3-((4-fluoro-2-methyl-1,1-dioxido-2,3-dihydrobenzo[d]isothiazol-5-yl)amino)-1H-pyrazol-5-yl)cyclopentyl(4-hydroxycyclohexyl)carbamate

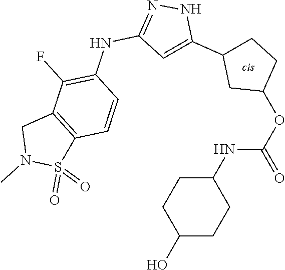 [SMILES: CN1Cc2c(ccc(Nc3cc(C4CCC(OC(=O)NC5CCC(O)CC5)C4)[nH]n3)c2F)S1(=O)=O]

The titled compound was synthesized in the procedures similar to Example 121.

Diastereoisomer 1 (Example 166): ¹H NMR (500 MHz, DMSO-d₆) δ 11.99 (s, 1H), 8.76 (s, 1H), 8.32-8.22 (m, 1H), 7.54 (t, J=10 Hz, 1H), 6.92 (t, J=5 Hz, 1H), 5.83 (s, 1H), 5.03-4.95 (m, 1H), 4.50-4.45 (m, 1H), 4.42 (s, 2H), 3.29-3.27 (m, 1H), 3.24-3.14 (m, 1H), 3.11-3.01 (m, 1H), 2.78 (s, 3H), 2.47-2.42 (m, 1H), 2.06-1.98 (m, 1H), 1.95-1.86 (m, 1H), 1.82-1.53 (m, 7H), 1.22-1.08 (m, 4H). LC-MS (ESI): m/z [M+H]⁺=508.2.

Diastereoisomer 2 (Example 167): ¹H NMR (500 MHz, DMSO-d₆) δ 11.99 (s, 1H), 8.76 (s, 1H), 8.33-8.19 (m, 1H), 7.54 (t, J=10 Hz, 1H), 6.96 (t, J=5 Hz, 1H), 5.84 (s, 1H), 5.06-4.95 (m, 1H), 4.42 (s, 2H), 4.32-4.21 (m, 1H), 3.70-3.61 (m, 1H), 3.27-3.22 (m, 1H), 3.11-3.02 (m, 1H), 2.78 (s, 3H), 2.43-2.28 (m, 1H), 2.06-1.97 (m, 1H), 1.97-1.86 (m, 1H), 1.79-1.66 (m, 2H), 1.62-1.34 (m, 9H). LC-MS (ESI): m/z [M+H]⁺=508.2.

### Example 168: cis-3-(3-((4-fluoro-2-methyl-1,1-dioxido-2,3-dihydrobenzo[d]isothiazol-5-yl)amino)-1H-pyrazol-5-yl)cyclopentyl(2-hydroxy-2-methylpropyl)carbamate

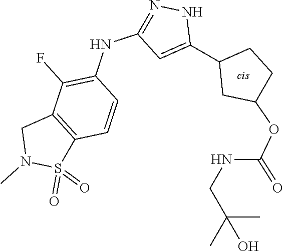 [SMILES: CN1Cc2c(ccc(Nc3cc(C4CCC(OC(=O)NCC(C)(C)O)C4)[nH]n3)c2F)S1(=O)=O]

The titled compound was synthesized in the procedures similar to Example 121. ¹H NMR (500 MHz, DMSO-d₆) δ 11.99 (s, 1H), 8.76 (s, 1H), 8.28 (t, J=5 Hz, 1H), 7.54 (d, J=10 Hz, 1H), 6.84 (t, J=5 Hz, 1H), 5.85 (s, 1H), 5.03-4.95 (m, 1H), 4.42 (s, 2H), 4.33 (s, 1H), 3.12-3.04 (m, 1H), 2.96-2.89 (m, 2H), 2.78 (s, 3H), 2.48-2.43 (m, 1H), 2.07-1.98 (m, 1H), 1.96-1.85 (m, 1H), 1.80-1.68 (m, 2H), 1.66-1.57 (m, 1H), 1.02 (s, 6H). LC-MS (ESI): m/z [M+H]⁺=482.2.

### Example 169: cis-3-(3-((4-fluoro-2-methyl-1,1-dioxido-2,3-dihydrobenzo[d]isothiazol-5-yl)amino)-1H-pyrazol-5-yl)cyclopentyl((1s,4s)-4-hydroxy-4-methylcyclohexyl)carbamate

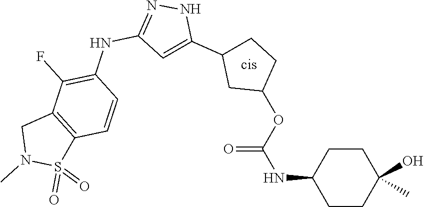

The titled compound was synthesized in the procedures similar to Example 121. ¹H NMR (500 MHz, DMSO-d₆) δ 11.99 (s, 1H), 8.77 (s, 1H), 8.35-8.21 (m 1H), 7.69 (t, J=5 Hz, 1H), 7.54 (d, J=10 Hz, 1H), 6.94 (d, J=10 Hz, 1H), 5.84 (s, 1H), 5.07-4.94 (m, 1H), 4.42 (s, 2H), 3.95 (s, 1H), 3.22-3.12 (m, 1H), 3.11-2.97 (m, 1H), 2.78 (s, 3H), 2.48-2.42 (m, 1H), 2.05-1.97 (m, 1H), 1.95-1.84 (m, 1H), 1.78-1.65 (m, 2H), 1.63-1.38 (m, 7H), 1.32-1.21 (m, 2H), 1.07 (s, 3H). LC-MS (ESI): m/z [M+H]⁺=522.2.

### Example 170: cis-3-(3-((4-fluoro-2-methyl-1,1-dioxido-2,3-dihydrobenzo[d]isothiazol-5-yl)amino)-1H-pyrazol-5-yl)cyclopentyl(2S,3R)-3-hydroxy-2-methylazetidine-1-carboxylate

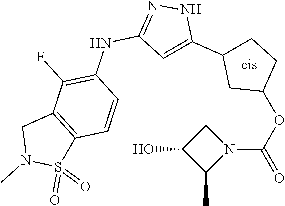 [SMILES: C[C@H]1[C@H](O)CN1C(=O)OC1CCC(c2cc(Nc3ccc4c(c3F)CN(C)S4(=O)=O)n[nH]2)C1]

The titled compound was synthesized in the procedures similar to Example 121. ¹H NMR (500 MHz, DMSO-d₆) δ 12.02 (s, 1H), 8.78 (s, 1H), 8.29 (s, 1H), 7.54 (d, J=8.6 Hz, 1H), 5.83 (s, 1H), 5.61 (d, J=6.0 Hz, 1H), 5.01 (s, 1H), 4.42 (s, 2H), 3.95-3.85 (m, 3H), 3.48 (s, 1H), 3.13-3.06 (m, 1H), 2.79 (s, 3H), 2.48-2.43 (m, 1H), 2.05-1.85 (m, 2H), 1.78-1.61 (m, 3H), 1.28-1.21 (m, 3H). LC-MS (ESI): m/z [M+H]⁺=480.2.

### Example 171: cis-3-(3-((1,1-dioxido-3-oxo-3,4-dihydro-2H-benzo[e][1,2]thiazin-6-yl)amino)-1H-pyrazol-5-yl)cyclopentyl isopropylcarbamate

### Step 1: methyl 2-(5-bromo-2-(chlorosulfonyl)phenyl) acetate

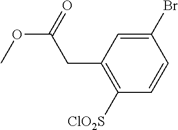 [SMILES: COC(=O)Cc1cc(Br)ccc1S(=O)(=O)Cl]

A mixture of methyl 2-(3-bromophenyl)acetate (1.5 g, 6.6 mmol) and Chlorosulfonic acid (8 mL) was stirred at rt for 12 hr. The mixture was quenched by ice water and extracted by EA (20 mL×3). The EA layer was washed by brine, dried over Na₂SO₄ and concentrated to afford the crude product (1.2 g, crude), which was used for next step without purification.

### Step 2:6-bromo-2H-benzo[e][1.2]thiazin-3 (4H)-one 1,1-dioxide

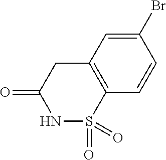 [SMILES: O=C1Cc2cc(Br)ccc2S(=O)(=O)N1]

To a solution of methyl 2-(5-bromo-2-(chlorosulfonyl)phenyl) acetate (1.2 g, crude) in THF (10 mL) was added Ammonium hydroxide (2.5 mL). The mixture was stirred at rt for 12 hr. The mixture was concentrated and triturated in EA (5 mL) for 1 hour. The mixture was filtered and the solid was dried to afford the product (0.5 g, 27.8%, over two steps). LC-MS (ESI): m/z [M+H]⁺=276.2

### Step 3: cis-3-(3-((1,1-dioxido-3-oxo-3,4-dihydro-2H-benzo[e][1.2]thiazin-6-yl)amino)-1H-pyrazol-5-yl)cyclopentyl isopropylcarbamate

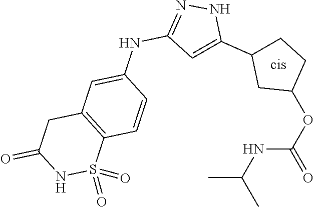 [SMILES: CC(C)NC(=O)OC1CCC(c2cc(Nc3ccc4c(c3)CC(=O)NS4(=O)=O)n[nH]2)C1]

The titled compound was synthesized in the procedures similar to Example 8, step 3 to step 4. ¹H NMR (500 MHz, DMSO-d₆) δ 12.60 (s, 1H), 11.99 (s, 1H), 9.10 (s, 1H), 7.64 (d, J=8.7 Hz, 1H), 7.42-7.32 (m, 2H), 6.98-6.91 (m, 1H), 5.73 (s, 1H), 5.00 (s, 1H), 3.91 (s, 2H), 3.62-3.53 (m, 1H), 3.10-3.01 (m, 1H), 2.48-2.40 (m, 1H), 2.07-1.98 (m, 1H), 1.95-1.87 (m, 1H), 1.80-1.65 (m, 2H), 1.64-1.56 (m, 1H), 1.09-0.96 (m, 6H). LC-MS (ESI): m/z [M+H]⁺=448.2.

### Example 172: cis-3-(3-((1,1-dioxido-3,4-dihydro-2H-benzo[e][1,2]thiazin-6-yl)amino)-1H-pyrazol-5-yl)cyclopentyl isopropylcarbamate

### Step 1:6-bromo-3,4-dihydro-2H-benzo[e][1,2]thiazine 1,1-dioxide

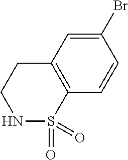 [SMILES: O=S1(=O)NCCc2cc(Br)ccc21]

To a stirring solution of 6-bromo-2H-benzo[e][1,2]thiazin-3 (4H)-one 1,1-dioxide (500 mg, 1.82 mmol) in THF (20 mL) was added BH₃-Me₂S (2M, 9 mL, 18 mmol) dropwise at 0° C. under N₂ atmosphere. The solution was stirred for 3 h at 75° C. The solution was cooled down to 0° C., quenched with MeOH (10 mL). The resulting mixture was concentrated under reduced pressure. The residue was purified by silica gel column chromatography, eluting with PE/EtOAc (1:1) to afford the product (350 mg, 73.8%). LC-MS (ESI): m/z [M+H]⁺=262.1.

### Step 2: cis-3-(3-((1,1-dioxido-3,4-dihydro-2H-benzo[e][1.2]thiazin-6-yl)amino)-1H-pyrazol-5-yl)cyclopentyl isopropylcarbamate

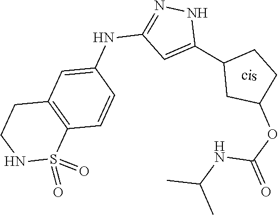 [SMILES: CC(C)NC(=O)OC1CCC(c2cc(Nc3ccc4c(c3)CCNS4(=O)=O)n[nH]2)C1]

The titled compound was synthesized in the procedures similar to Example 8, step 3 to step 4. ¹H NMR (500 MHz, DMSO-d₆) δ 11.86 (s, 1H), 8.79 (s, 1H), 7.47 (d, J=8.6 Hz, 1H), 7.25 (d, J=7.7 Hz, 2H), 7.14 (t, J=7.2 Hz, 1H), 6.95 (d, J=7.8 Hz, 1H), 5.67 (s, 1H), 4.99 (s, 1H), 3.65-3.53 (m, 1H), 3.50 (dd, J=12.4, 6.0 Hz, 2H), 3.09-3.02 (m, 1H), 2.81 (t, J=5.8 Hz, 2H), 2.45-2.42 (m, 1H), 2.05-1.98 (m, 1H), 1.95-1.85 (m, 1H), 1.78-1.65 (m, 2H), 1.63-1.55 (m, 1H), 1.03 (d, J=6.3 Hz, 6H).

LC-MS (ESI): m/z [M+H]⁺=434.2.

### Example 173:3-(3-((1,1-dioxido-2,3-dihydrobenzo[d]isothiazol-5-yl)amino)-1H-pyrazol-5-yl)cyclobutyl isopropylcarbamate

### Step 1: benzyl(1-(tert-butyl)-3-(3-((isopropylcarbamoyl)oxy)cyclobutyl)-1H-pyrazol-5-yl)carbamate

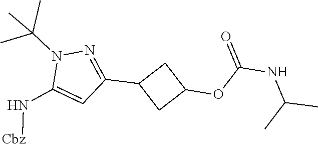 [SMILES: CC(C)NC(=O)OC1CC(c2cc(NC(=O)OCc3ccccc3)n(C(C)(C)C)n2)C1]

To a mixture of benzyl(1-(tert-butyl)-3-(3-(((4-nitrophenoxy) carbonyl)oxy)cyclobutyl)-1H-pyrazol-5-yl)carbamate (0.7 g, 1.38 mmol) and propan-2-amine (0.13 g, 2.07 mmol) in THF (15 mL), DIEA (0.36 g, 2.76 mmol) was added. The mixture was stirred at 25° C. for 12 hrs. The mixture was concentrated under reduced pressure. The residue was purified by silica gel column chromatography, eluting with DCM/MeOH (10:1) to afford the product (0.5 g, 59%). LC-MS (ESI): m/z [M+H]⁺=429.3.

### Step 2:3-(5-amino-1-(tert-butyl)-1H-pyrazol-3-yl)cyclobutyl isopropylcarbamate

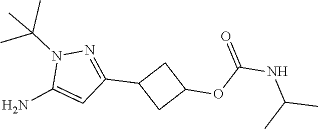 [SMILES: CC(C)NC(=O)OC1CC(c2cc(N)n(C(C)(C)C)n2)C1]

To a mixture of benzyl(1-(tert-butyl)-3-(3-((isopropylcarbamoyl)oxy)cyclobutyl)-1H-pyrazol-5-yl)carbamate (0.5 g, 1.17 mmol) in MeOH (10 mL), 10% Pd/C (0.1 g) was added. The mixture was stirred at 25° C. under H₂ balloon for 2 hrs. The mixture was filtered and washed by MeOH (100 mL). The filtrate was concentrated under reduced pressure to afford the crude product (0.4 g, crude), which was used for next step without purification. LC-MS (ESI): m/z [M+H]⁺=295.3.

### Step 3:3-(3-((1,1-dioxido-2,3-dihydrobenzo[d]isothiazol-5-yl)amino)-1H-pyrazol-5-yl)cyclobutyl isopropylcarbamate

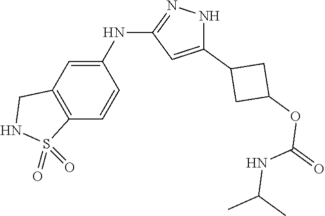 [SMILES: CC(C)NC(=O)OC1CC(c2cc(Nc3ccc4c(c3)CNS4(=O)=O)n[nH]2)C1]

The titled compound was synthesized in the procedures similar to Example 50. ¹H NMR (500 MHz, DMSO-d₆) δ 11.99 (s, 1H), 9.02 (s, 1H), 7.53 (d, J=8.6 Hz, 1H), 7.46 (t, J=4.8 Hz, 2H), 7.30 (d, J=7.9 Hz, 1H), 7.05 (d, J=7.9 Hz, 1H), 5.76 (s, 1H), 4.80 (p, J=7.7 Hz, 1H), 4.29 (d, J=4.9 Hz, 2H), 3.57 (dq, J=13.7, 6.9 Hz, 1H), 3.10-3.01 (m, 1H), 2.72-2.63 (m, 2H), 2.13-2.03 (m, 2H), 1.04 (d, J=6.6 Hz, 6H). LC-MS (ESI): m/z [M+H]⁺=406.2.

### Example 174: cis-3-(3-((1,1-dioxido-2,3-dihydrobenzo[d]isothiazol-5-yl)amino)-1H-pyrazol-5-yl)cyclopentyl pyrrolidine-1-carboxylate

### Step 1: tert-butyl 5-bromobenzo[d]isothiazole-2 (3H)-carboxylate 1,1-dioxide

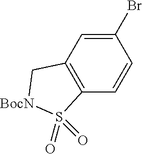 [SMILES: CC(C)(C)OC(=O)N1Cc2cc(Br)ccc2S1(=O)=O]

To a solution of 5-bromo-2,3-dihydrobenzo[d]isothiazole 1,1-dioxide (325 mg, 1.3 mmol) in THF (20 mL) was added di-tert-butyl dicarbonate (327 mg, 1.5 mmol) and DMAP (12 mg, 0.1 mmol). The resulting mixture was stirred at room temperature for 1 h. The solvent was removed under vacuum and the residue was purified by silica gel chromatography, eluting with PE/EA (2:1) to afford the product (500 mg, 95%). LC-MS (ESI): m/z [M+H]⁺=348.2.

### Step 2: cis-tert-butyl 5-((1-(tert-butyl)-3-(3-hydroxycyclopentyl)-1H-pyrazol-5-yl)amino)benzo[d]isothiazole-2 (3H)-carboxylate 1,1-dioxide

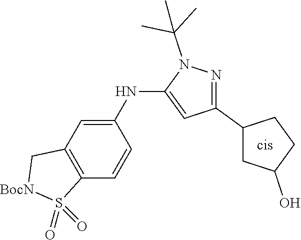 [SMILES: CC(C)(C)OC(=O)N1Cc2cc(Nc3cc(C4CCC(O)C4)nn3C(C)(C)C)ccc2S1(=O)=O]

To a mixture of tert-butyl 5-bromobenzo[d]isothiazole-2 (3H)-carboxylate 1,1-dioxide (380 mg, 1.1 mmol) and cis-3-(5-amino-1-(tert-butyl)-1H-pyrazol-3-yl)cyclopentan-1-ol (291 mg, 1.3 mmol) in andydrous t-BuOH (10 mL) was added BrettPhos Pd G3 (74.4 mg, 0.08 mmol) and K₃PO₄ (827 mg, 3.9 mmol). The reaction mixture was heated to 110° C. and stirred under nitrogen atmosphere for 3 h. The mixture was concentrated under reduced pressure. The residue was purified by silica gel column chromatography, eluting with PE/EA (1:5) to afford the product (430 mg, 88%). LC-MS (ESI): m/z [M+H]⁺=491.3.

### Step 3: cis-tert-butyl 5-((1-(tert-butyl)-3-(3-(((4-nitrophenoxy) carbonyl)oxy) cyclopentyl)-1H-pyrazol-5-yl)amino)benzo[d]isothiazole-2 (3H)-carboxylate 1,1-dioxide

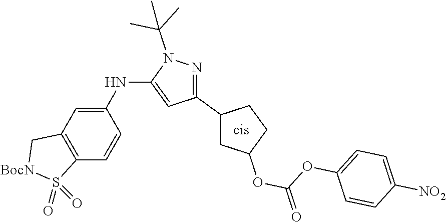 [SMILES: CC(C)(C)OC(=O)N1Cc2cc(Nc3cc(C4CCC(OC(=O)Oc5ccc([N+](=O)[O-])cc5)C4)nn3C(C)(C)C)ccc2S1(=O)=O]

To a solution of cis-tert-butyl 5-((1-(tert-butyl)-3-(3-hydroxycyclopentyl)-1H-pyrazol-5-yl)amino)benzo[d]isothiazole-2 (3H)-carboxylate 1,1-dioxide (430 mg, 0.85 mmol) in THF (20 mL) was added pyridine (332 mg, 4.2 mmol), DMAP (10 mg, 0.08 mmol), 4-nitrophenyl carbonochloridate (342 mg, 1.7 mmol), successively. The resulting mixture was stirred at 50° C. for 48 h. The reaction mixture was concentrated under reduced pressure, and the residue was purified by silica gel chromatography, eluting with PE/EA (3:1) to afford the product (456 mg, 82%). LC-MS (ESI): m/z [M+H]⁺=656.3.

### Step 4: cis-tert-butyl 5-((1-(tert-butyl)-3-(3-((pyrrolidine-1-carbonyl)oxy) cyclopentyl)-1H-pyrazol-5-yl)amino)benzo[d]isothiazole-2 (3H)-carboxylate 1,1-dioxide

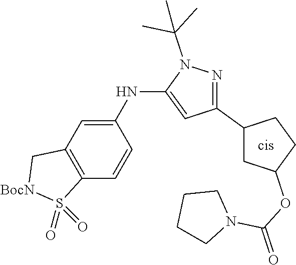 [SMILES: CC(C)(C)OC(=O)N1Cc2cc(Nc3cc(C4CCC(OC(=O)N5CCCC5)C4)nn3C(C)(C)C)ccc2S1(=O)=O]

To a stirred solution of cis-tert-butyl 5-((1-(tert-butyl)-3-(3-(((4-nitrophenoxy) carbonyl)oxy) cyclopentyl)-1H-pyrazol-5-yl)amino)benzo[d]isothiazole-2 (3H)-carboxylate 1,1-dioxide (61 mg, 0.09 mmol) in THF (10 mL) was added pyrrolidine (21 mg, 0.3 mmol). The resulting mixture was stirred at room temperature for 1 h. The resulting mixture was concentrated under reduced pressure, and the residue was purified by silica gel column chromatography, eluting with PE/EA (2:1) to afford the product (52 mg, 98%). LC-MS (ESI): m/z [M+H]⁺=588.3.

### Step 5: cis-3-(3-((1,1-dioxido-2,3-dihydrobenzo[d]isothiazol-5-yl)amino)-1H-pyrazol-5-yl)cyclopentyl pyrrolidine-1-carboxylate

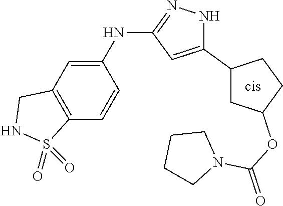 [SMILES: O=C(OC1CCC(c2cc(Nc3ccc4c(c3)CNS4(=O)=O)n[nH]2)C1)N1CCCC1]

To a solution of cis-tert-butyl 5-((1-(tert-butyl)-3-(3-((pyrrolidine-1-carbonyl)oxy) cyclopentyl)-1H-pyrazol-5-yl)amino)benzo[d]isothiazole-2 (3H)-carboxylate 1,1-dioxide (40 mg, 0.08 mmol) in DCM (1.5 mL) was added trifluoromethanesulfonic acid (3 drops). The reaction mixture was stirred at room temperature for 2 h. The resulting mixture was neutralized with sat. aq. NaHCO₃ and extracted with DCM (5 mL×3). The combined organic layers were washed with brine, dried over NaSO₄, filtered, and concentrated under reduce pressure. The residue was purified by prep-HPLC (Waters SunFire C18: 19×150 mm, 5 μm, eluting with 33%-45% of acetonitrile (containing 0.1% FA) in water (containing 0.1% FA)) to afford the product (15.0 mg, 43%). ¹H NMR (500 MHz, DMSO-d₆) δ 11.91 (s, 1H), 8.99 (s, 1H), 8.16-8.08 (m 1H), 7.52 (d, J=10 Hz, 1H), 7.49-7.42 (m, 1H), 7.30 (d, J=10 Hz, 1H), 6.93 (d, J=10 Hz, 1H), 5.70 (s, 1H), 5.06-4.99 (m, 1H), 4.28 (d, J=5 Hz, 1H), 3.25-3.16 (m, 4H), 3.14-3.06 (m, 1H), 2.45-2.38 (m, 1H), 2.07-1.98 (m, 1H), 1.95-1.86 (m, 1H), 1.81-1.65 (m, 7H). LC-MS (ESI): m/z [M+H]⁺=432.2.

### Example 175: cis-3-(3-((1,1-dioxido-2,3-dihydrobenzo[d]isothiazol-5-yl)amino)-1H-pyrazol-5-yl)cyclopentyl 3-hydroxypyrrolidine-1-carboxylate

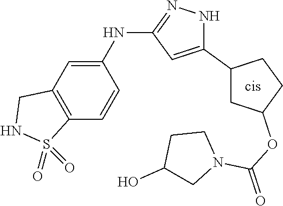 [SMILES: O=C(OC1CCC(c2cc(Nc3ccc4c(c3)CNS4(=O)=O)n[nH]2)C1)N1CCC(O)C1]

The titled compound was synthesized in the procedures similar to Example 174. ¹H NMR (500 MHz, DMSO-d₆) δ 11.93 (s, 1H), 9.00 (s, 1H), 7.55-7.50 (m, 1H), 7.48-7.43 (m, 2H), 7.30 (d, J=10 Hz, 1H), 5.71 (s, 1H), 5.06-5.00 (m, 1H), 4.93-4.89 (m, 1H), 4.28 (d, J=5 Hz, 2H), 4.24-4.19 (m, 1H), 3.30-3.26 (m, 3H), 3.17-3.05 (m, 2H), 2.48-2.38 (m, 1H), 2.06-1.98 (m, 1H), 1.96-1.65 (m, 6H). LC-MS (ESI): m/z [M+H]⁺=448.2.

### Example 176: cis-3-(3-((1,1-dioxido-2,3-dihydrobenzo[d]isothiazol-5-yl)amino)-1H-pyrazol-5-yl)cyclopentyl 3-hydroxy-3-methylpyrrolidine-1-carboxylate

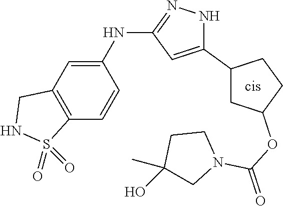 [SMILES: CC1(O)CCN(C(=O)OC2CCC(c3cc(Nc4ccc5c(c4)CNS5(=O)=O)n[nH]3)C2)C1]

The titled compound was synthesized in the procedures similar to Example 174. ¹H NMR (500 MHz, DMSO-d₆) δ 11.92 (s, 1H), 9.00 (s, 1H), 7.52 (d, J=10 Hz, 1H), 7.49-7.43 (m, 2H), 7.30 (d, J=10 Hz, 1H), 5.71 (s, 1H), 5.06-4.98 (m, 1H), 4.76-4.71 (m, 1H), 4.28 (d, J=5 Hz, 2H), 3.36-3.34 (m, 2H), 3.23-3.14 (m, 1H), 3.13-3.052 (m, 2H), 2.46-2.38 (m, 1H), 2.07-1.98 (m, 1H), 1.96-1.87 (m, 1H), 1.82-1.64 (m, 5H), 1.27-1.18 (m, 3H). LC-MS (ESI): m/z [M+H]⁺=462.2.

### Example 177: cis-3-(3-((1,1-dioxido-2,3-dihydrobenzo[d]isothiazol-5-yl)amino)-1H-pyrazol-5-yl)cyclopentyl(1-methylcyclopropyl)carbamate

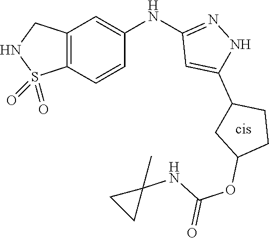 [SMILES: CC1(NC(=O)OC2CCC(c3cc(Nc4ccc5c(c4)CNS5(=O)=O)n[nH]3)C2)CC1]

The titled compound was synthesized in the procedures similar to Example 174. ¹H NMR (500 MHz, DMSO-d₆) δ 11.94 (s, 1H), 9.00 (s, 1H), 7.52 (d, J=10 Hz, 1H), 7.45 (s, 1H), 7.36-7.26 (m, 2H), 5.71 (s, 1H), 5.10-4.98 (m, 1H), 4.76-4.71 (m, 1H), 4.29 (s, 2H), 3.13-2.98 (m, 1H), 2.47-2.41 (m, 1H), 2.09-1.96 (m, 1H), 1.95-1.85 (m, 1H), 1.76-1.64 (m, 2H), 1.62-1.53 (m, 1H), 1.23 (s, 3H), 0.61-0.55 (m, 2H), 0.49-0.44 (m, 2H). LC-MS (ESI): m/z [M+H]⁺=432.2.

### Example 178: cis-3-(3-((1,1-dioxido-2,3-dihydrobenzo[d]isothiazol-5-yl)amino)-1H-pyrazol-5-yl)cyclopentyl propylcarbamate

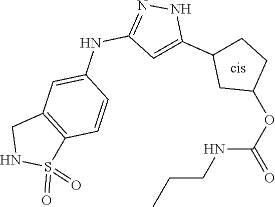 [SMILES: CCCNC(=O)OC1CCC(c2cc(Nc3ccc4c(c3)CNS4(=O)=O)n[nH]2)C1]

The titled compound was synthesized in the procedures similar to Example 174. ¹H NMR (500 MHz, DMSO-d₆) δ 11.90 (s, 1H), 9.00 (s, 1H), 7.53 (d, J=8.6 Hz, 1H), 7.50-7.43 (m, 2H), 7.30 (d, J=8.2 Hz, 1H), 7.05 (t, J=5.4 Hz, 1H), 5.70 (s, 1H), 5.00 (s, 1H), 4.29 (d, J=5.0 Hz, 2H), 3.12-3.02 (m, 1H), 2.91 (dd, J=13.2, 6.7 Hz, 2H), 2.48-2.42 (m, 1H), 2.09-1.98 (m, 1H), 1.95-1.85 (m, 1H), 1.78-1.57 (m, 3H), 1.43-1.31 (m, 2H), 0.82 (t, J=7.4 Hz, 3H). LC-MS (ESI): m/z [M+H]⁺=420.3.

### Example 179: cis-3-(3-((4-fluoro-1,1-dioxido-2,3-dihydrobenzo[d]isothiazol-6-yl)amino)-1H-pyrazol-5-yl)cyclopentyl isopropylcarbamate

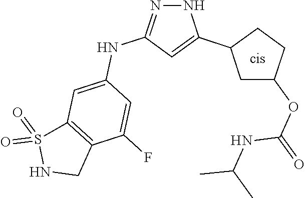 [SMILES: CC(C)NC(=O)OC1CCC(c2cc(Nc3cc(F)c4c(c3)S(=O)(=O)NC4)n[nH]2)C1]

The titled compound was synthesized in the procedures similar to Example 34. ¹H NMR (500 MHz, DMSO-d₆)) δ 11.85 (s, 1H), 9.05 (s, 1H), 7.79 (t, J=4.8 Hz, 1H), 7.60 (s, 1H), 7.32 (d, J=11.9 Hz, 1H), 6.88 (d, J=7.4 Hz, 1H), 5.57 (s, 1H), 4.93 (s, 1H), 4.23 (d, J=5.0 Hz, 2H), 3.51 (dd, J=12.6, 6.4 Hz, 1H), 3.13-2.88 (m, 1H), 2.41-2.33 (m, 1H), 2.03-1.91 (m, 1H), 1.90-1.78 (m, 1H), 1.70-1.60 (m, 2H), 1.58-1.48 (m, 1H), 0.96 (d, J=6.2 Hz, 6H). LC-MS (ESI): m/z [M+H]⁺=438.2

### Example 180: cis-3-(3-((7-methoxy-1,1-dioxido-2,3-dihydrobenzo[d]isothiazol-6-yl)amino)-1H-pyrazol-5-yl)cyclopentyl isopropylcarbamate

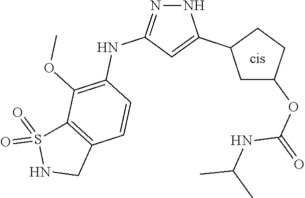 [SMILES: COc1c(Nc2cc(C3CCC(OC(=O)NC(C)C)C3)[nH]n2)ccc2c1S(=O)(=O)NC2]

The titled compound was synthesized in the procedures similar to Example 34. ¹H NMR (500 MHz, DMSO-d₆) δ=11.84 (s, 1H), 8.27 (s, 1H), 7.99 (s, 1H), 7.69 (s, 1H), 7.08 (d, J=8.4, 1H), 6.94 (d, J=7.4, 1H), 5.83 (s, 1H), 5.00 (s, 1H), 4.23 (d, J=3.2, 2H), 3.58 (m, 1H), 3.10-2.95 (m, 1H), 2.45 (m, 1H), 2.02 (m, 1H), 1.90 (m, 1H), 1.70 (m, 2H), 1.60 (m, 1H), 1.08-0.92 (m, 6H). LC-MS (ESI): m/z [M+H]⁺=450.3.

### Example 181: cis-3-(3-((7-methoxy-3,3-dimethyl-1,1-dioxido-2,3-dihydrobenzo[d]isothiazol-6-yl)amino)-1H-pyrazol-5-yl)cyclopentyl isopropylcarbamate

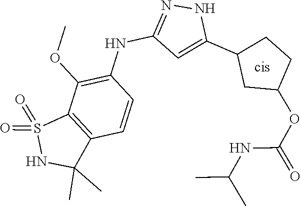 [SMILES: COc1c(Nc2cc(C3CCC(OC(=O)NC(C)C)C3)[nH]n2)ccc2c1S(=O)(=O)NC2(C)C]

The titled compound was synthesized in the procedures similar to Example 6. ¹H NMR (500 MHz, DMSO-d₆) δ=11.87 (s, 1H), 8.24 (s, 1H), 7.99 (s, 1H), 7.73 (s, 1H), 7.18 (d, J=8.5, 1H), 6.94 (d, J=7.5, 1H), 5.83 (s, 1H), 5.00 (s, 1H), 3.90 (s, 3H), 3.58 (m, 1H), 3.05 (m, 1H), 2.45 (m, 1H), 2.02 (m, 1H), 1.90 (m, 1H), 1.70 (m, 2H), 1.61 (m, 1H), 1.47 (s, 6H), 1.08-0.94 (m, 6H). LC-MS (ESI): m/z [M+H]⁺=478.6.

### Example 182: cis-3-(3-((5-methoxy-1,1-dioxido-2,3-dihydrobenzo[d]isothiazol-6-yl)amino)-1H-pyrazol-5-yl)cyclopentyl isopropylcarbamate

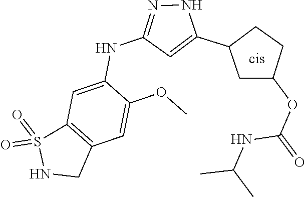 [SMILES: COc1cc2c(cc1Nc1cc(C3CCC(OC(=O)NC(C)C)C3)[nH]n1)S(=O)(=O)NC2]

The titled compound was synthesized in the procedures similar to Example 8. ¹H NMR (500 MHz, DMSO-d₆) δ 11.81 (s, 1H), 8.45 (s, 1H), 7.95 (s, 1H), 7.51 (s, 1H), 7.02 (s, 1H), 6.95 (d, J=7.2 Hz, 1H), 5.84 (s, 1H), 5.00 (s, 1H), 4.26 (s, 2H), 3.93 (s, 3H), 3.61-3.54 (m, 1H), 3.09-3.01 (m, 1H), 2.48-2.42 (m, 1H), 2.08-1.97 (m, 1H), 1.96-1.85 (m, 1H), 1.77-1.65 (m, 2H), 1.64-1.55 (m, 1H), 1.03 (d, J=6.3 Hz, 6H). LC-MS (ESI): m/z [M+H]⁺=450.2.

### Example 183: cis-3-(3-((5-methoxy-3,3-dimethyl-1,1-dioxido-2,3-dihydrobenzo[d]isothiazol-6-yl)amino)-1H-pyrazol-5-yl)cyclopentyl isopropylcarbamate

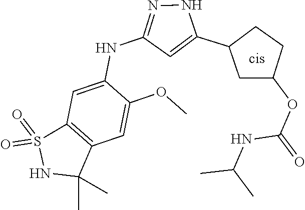 [SMILES: COc1cc2c(cc1Nc1cc(C3CCC(OC(=O)NC(C)C)C3)[nH]n1)S(=O)(=O)NC2(C)C]

The titled compound was synthesized in the procedures similar to Example 6. ¹H NMR (500 MHz, DMSO-d₆) δ 11.80 (s, 1H), 8.38 (s, 1H), 7.91 (s, 1H), 7.56 (s, 1H), 7.11 (s, 1H), 6.96 (d, J=7.1 Hz, 1H), 5.84 (s, 1H), 5.00 (s, 1H), 3.96 (s, 3H), 3.65-3.50 (m, 1H), 3.11-3.01 (m, 1H), 2.48-2.40 (m, 1H), 2.08-1.99 (m, 1H), 1.93-1.84 (m, 1H), 1.78-1.65 (m, 2H), 1.64-1.55 (m, 1H), 1.49 (s, 6H), 1.03 (d, J=6.3 Hz, 6H). LC-MS (ESI): m/z [M+H]⁺=478.2.

### Example 184: cis-3-(3-((4-chloro-1,1-dioxido-2,3-dihydrobenzo[d]isothiazol-6-yl)amino)-1H-pyrazol-5-yl)cyclopentyl isopropylcarbamate

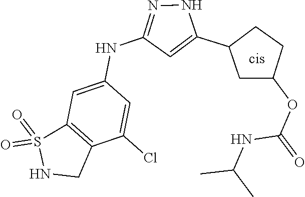 [SMILES: CC(C)NC(=O)OC1CCC(c2cc(Nc3cc(Cl)c4c(c3)S(=O)(=O)NC4)n[nH]2)C1]

The titled compound was synthesized in the procedures similar to Example 34. ¹H NMR (500 MHz, DMSO-d₆) δ 11.93 (s, 1H), 9.11 (s, 1H), 7.90 (t, J=4.8 Hz, 1H), 7.83 (s, 1H), 7.66 (s, 1H), 6.95 (d, J=7.2 Hz, 1H), 5.64 (s, 1H), 5.00 (s, 1H), 4.24 (d, J=4.9 Hz, 2H), 3.58 (td, J=11.4, 4.9 Hz, 1H), 3.13-3.00 (m, 1H), 2.48-2.42 (m, 1H), 2.02 (dd, J=15.5, 7.6 Hz, 1H), 1.90 (dq, J=12.7, 6.6 Hz, 1H), 1.72 (dd, J=20.0, 11.3 Hz, 2H), 1.60 (td, J=14.0, 5.0 Hz, 1H), 1.03 (d, J=6.3 Hz, 6H). LC-MS (ESI): m/z [M+H]⁺=454.2.

### Example 185: cis-3-(3-((5-methyl-1,1-dioxido-2,3-dihydrobenzo[d]isothiazol-6-yl)amino)-1H-pyrazol-5-yl)cyclopentyl isopropylcarbamate

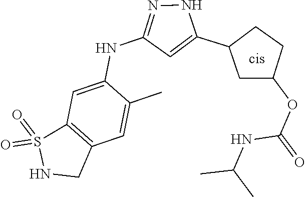 [SMILES: Cc1cc2c(cc1Nc1cc(C3CCC(OC(=O)NC(C)C)C3)[nH]n1)S(=O)(=O)NC2]

The titled compound was synthesized in the procedures similar to Example 34. ¹H NMR (500 MHz, DMSO-d₆) δ 11.87 (s, 1H), 8.32 (s, 1H), 7.66 (s, 1H), 7.55 (s, 1H), 7.21 (s, 1H), 6.93 (d, J=6.6 Hz, 1H), 5.82 (s, 1H), 5.00 (s, 1H), 4.24 (d, J=4.6 Hz, 2H), 3.62-3.53 (m, 1H), 3.13-3.04 (m, 1H), 2.48-2.43 (m, 1H), 2.32 (s, 3H), 2.05-1.87 (m, 2H), 1.78-1.55 (m, 3H), 1.03 (d, J=6.0 Hz, 6H). LC-MS (ESI): m/z [M+H]⁺=434.2.

### Example 186: cis-3-(3-((7-fluoro-1,1-dioxido-2,3-dihydrobenzo[d]isothiazol-6-yl)amino)-1H-pyrazol-5-yl)cyclopentyl isopropylcarbamate

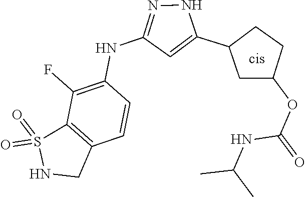 [SMILES: CC(C)NC(=O)OC1CCC(c2cc(Nc3ccc4c(c3F)S(=O)(=O)NC4)n[nH]2)C1]

The titled compound was synthesized in the procedures similar to Example 34 in a racemic form, which was further separated by Chiral Prep-HPLC to give:

Enantiomer 1 (Example 186a, 100% ee); Retention time: 3.052 min. ¹H NMR (500 MHz, DMSO-d₆) δ=11.89 (s, 1H), 8.48 (s, 1H), 8.35 (s, 1H), 7.96 (t, J=5.1, 1H), 7.18 (d, J=8.5, 1H), 6.94 (d, J=7.4, 1H), 5.77 (s, 1H), 5.00 (s, 1H), 4.31 (d, J=5.2, 2H), 3.62-3.54 (m, 1H), 3.10-3.01 (m, 1H), 2.47-2.42 (m, 1H), 2.02 (m, 1H), 1.96-1.85 (m, 1H), 1.70 (m, 2H), 1.60 (m, 1H), 1.03 (m, 6H). LC-MS (ESI): m/z [M+H]⁺=438.3.

Enantiomer 2 (Example 186b, 100% ee); Retention time: 4.368 min. ¹H NMR (500 MHz, DMSO-d₆) δ=11.89 (s, 1H), 8.47 (s, 1H), 8.35 (s, 1H), 7.96 (t, J=5.2, 1H), 7.18 (d, J=8.5, 1H), 6.94 (d, J=7.1, 1H), 5.77 (s, 1H), 5.00 (s, 1H), 4.31 (d, J=5.2, 2H), 3.58 (m, 1H), 3.10-2.98 (m, 1H), 2.48-2.40 (m, 1H), 2.02 (m, 1H), 1.97-1.84 (m, 1H), 1.70 (m, 2H), 1.59 (s, 1H), 1.03 (m, 6H). LC-MS (ESI): m/z [M+H]⁺=438.4.

Chiral analytical method: Column: CHIRAL Cellulose-SB 4.6×100 mm, 3 μm; Mobile phase: A for MtBE (0.1% DEA) and B for EtOH; Gradient: Mobile Phase A: Mobile Phase B=90: 10 (v/v); Column temperature: 25° C.

Chiral Prep-HPLC Condition: CHIRAL Cellulose-SB 2 cm×25 cm, 5 μm; Mobile phase: A for MtBE and B for EtOH; Gradient: Mobile Phase A: Mobile Phase B=90: 10 (v/v); Flow Rate: 20 mL/min, Wavelength: UV 220 nm, Prep-HPLC Equipment: Prep-HPLC-Gilson; Column temperature: 25° C.

### Example 187: cis-3-(3-((7-fluoro-1,1-dioxido-2,3-dihydrobenzo[d]isothiazol-6-yl)amino)-1H-pyrazol-5-yl)cyclopentyl(1-methylcyclopropyl)carbamate

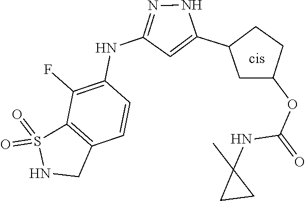 [SMILES: CC1(NC(=O)OC2CCC(c3cc(Nc4ccc5c(c4F)S(=O)(=O)NC5)n[nH]3)C2)CC1]

The titled compound was synthesized in the procedures similar to Example 34. ¹H NMR (500 MHz, DMSO-d₆) δ=11.87 (s, 1H), 8.47 (s, 1H), 8.34 (s, 1H), 7.96 (t, J=5.1, 1H), 7.34 (s, 1H), 7.18 (d, J=8.5, 1H), 5.76 (s, 1H), 4.99 (s, 1H), 4.30 (d, J=5.4, 2H), 3.05 (m, 1H), 2.45 (m, 1H), 2.01 (m, 1H), 1.96-1.85 (m, 1H), 1.69 (m, 2H), 1.57 (m, 1H), 1.24 (s, 3H), 0.59 (m, 2H), 0.47 (m, 2H). LC-MS (ESI): m/z [M+H]⁺=450.1.

### Example 188: cis-3-(3-((7-fluoro-1,1-dioxido-2,3-dihydrobenzo[d]isothiazol-6-yl)amino)-1H-pyrazol-5-yl)cyclopentyl((S)-sec-butyl)carbamate

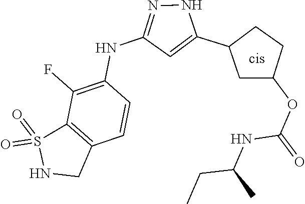 [SMILES: CC[C@H](C)NC(=O)OC1CCC(c2cc(Nc3ccc4c(c3F)S(=O)(=O)NC4)n[nH]2)C1]

The titled compound was synthesized in the procedures similar to Example 34. ¹H NMR (500 MHz, DMSO-d₆) δ=11.80 (s, 1H), 8.39 (s, 1H), 8.27 (s, 1H), 7.89 (t, J=5.2, 1H), 7.10 (d, J=8.5, 1H), 6.81 (d, J=8.1, 1H), 5.70 (s, 1H), 4.93 (s, 1H), 4.23 (d, J=5.4, 2H), 3.36-3.28 (m, 1H), 3.04-2.91 (m, 1H), 2.41-2.34 (m, 1H), 1.95 (m, 1H), 1.86 (m, 1H), 1.72-1.57 (m, 2H), 1.52 (m, 1H), 1.29 (m, 2H), 1.00-0.86 (m, 3H), 0.73 (m, 3H). LC-MS (ESI): m/z [M+H]⁺=452.2.

### Example 189: cis-3-(3-((7-fluoro-1,1-dioxido-2,3-dihydrobenzo[d]isothiazol-6-yl)amino)-1H-pyrazol-5-yl)cyclopentyl propylcarbamate

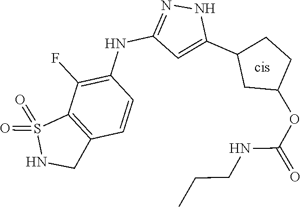 [SMILES: CCCNC(=O)OC1CCC(c2cc(Nc3ccc4c(c3F)S(=O)(=O)NC4)n[nH]2)C1]

The titled compound was synthesized in the procedures similar to Example 34 in racemic form, which was further separated by Chiral Prep-HPLC to give:

Enantiomer 1 (Example 189a, 100% ee); Retention time: 7.09 min. ¹H NMR (500 MHz, DMSO-d₆) δ 11.88 (s, 1H), 8.47 (s, 1H), 8.36 (t, J=7.5 Hz, 1H), 7.96 (s, 1H), 7.17 (d, J=8.4 Hz, 1H), 7.04 (s, 1H), 5.77 (s, 1H), 5.00 (s, 1H), 4.30 (s, 2H), 3.14-2.99 (m, 1H), 2.94-2.86 (m, 2H), 2.50-2.46 (m, 1H), 2.06-1.99 (m, 1H), 1.96-1.86 (m, 1H), 1.78-1.66 (m, 2H), 1.65-1.57 (m, 1H), 1.42-1.35 (m, 2H), 0.82 (t, J=7.2 Hz, 3H). LC-MS (ESI): m/z [M+H]⁺=438.2.

Enantiomer 2 (Example 189b, 100% ee); Retention time: 8.52 min. ¹H NMR (500 MHz, DMSO-d₆) δ 11.88 (s, 1H), 8.47 (s, 1H), 8.36 (t, J=7.5 Hz, 1H), 7.96 (s, 1H), 7.17 (d, J=8.4 Hz, 1H), 7.04 (s, 1H), 5.77 (s, 1H), 5.00 (s, 1H), 4.30 (s, 2H), 3.14-2.99 (m, 1H), 2.94-2.86 (m, 2H), 2.50-2.46 (m, 1H), 2.06-1.99 (m, 1H), 1.96-1.86 (m, 1H), 1.78-1.66 (m, 2H), 1.65-1.57 (m, 1H), 1.42-1.35 (m, 2H), 0.82 (t, J=7.2 Hz, 3H). LC-MS (ESI): m/z [M+H]⁺=438.2.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for Hexane and B for EtOH (0.2% 2M NH₃ MeOH); Gradient: Mobile Phase A: Mobile Phase B=40: 60 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 190: cis-3-(3-((7-fluoro-2-methyl-1,1-dioxido-2,3-dihydrobenzo[d]isothiazol-6-yl)amino)-1H-pyrazol-5-yl)cyclopentyl isopropylcarbamate

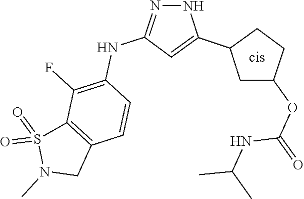 [SMILES: CC(C)NC(=O)OC1CCC(c2cc(Nc3ccc4c(c3F)S(=O)(=O)N(C)C4)n[nH]2)C1]

The titled compound was synthesized in the procedures similar to Example 121. ¹H NMR (500 MHz, DMSO-d₆) δ=11.91 (s, 1H), 8.53 (s, 1H), 8.39 (s, 1H), 7.23 (d, J=8.5, 1H), 6.94 (d, J=7.2, 1H), 5.77 (s, 1H), 5.00 (s, 1H), 4.33 (s, 2H), 3.58 (m, 1H), 3.11-3.00 (m, 1H), 2.81 (s, 3H), 2.46 (m, 1H), 2.02 (m, 1H), 1.97-1.84 (m, 1H), 1.70 (m, 2H), 1.60 (m, 1H), 1.03 (m, 6H). LC-MS (ESI): m/z [M+H]⁺=452.2.

### Example 191: cis-3-(3-((7-fluoro-2-(2-methoxyethyl)-1,1-dioxido-2,3-dihydrobenzo[d]isothiazol-6-yl)amino)-1H-pyrazol-5-yl)cyclopentyl isopropylcarbamate

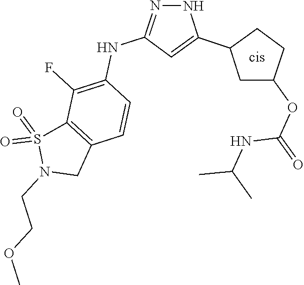 [SMILES: COCCN1Cc2ccc(Nc3cc(C4CCC(OC(=O)NC(C)C)C4)[nH]n3)c(F)c2S1(=O)=O]

The titled compound was synthesized in the procedures similar to Example 35. ¹H NMR (500 MHz, DMSO-d₆) δ 11.91 (s, 1H), 8.53 (s, 1H), 8.38 (d, J=5 Hz, 1H), 7.23 (d, J=10 Hz, 1H), 6.94 (d, J=10 Hz, 1H), 5.77 (s, 1H), 5.06-4.95 (m, 1H), 4.40 (s, 2H), 3.65-3.55 (m, 3H), 3.37-3.33 (m, 2H), 3.30 (s, 3H), 3.11-2.99 (m, 1H), 2.48-2.43 (m, 1H), 2.06-1.99 (m, 1H), 1.96-1.86 (m, 1H), 1.78-1.66 (m, 2H), 1.64-1.55 (m, 1H), 1.08-0.97 (m, 6H). LC-MS (ESI): m/z [M+H]⁺=496.2.

### Example 192: cis-3-(3-((7-fluoro-2-(2-hydroxyethyl)-1,1-dioxido-2,3-dihydrobenzo[d]isothiazol-6-yl)amino)-1H-pyrazol-5-yl)cyclopentyl isopropylcarbamate

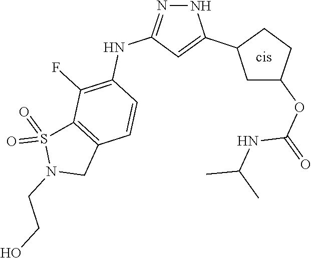 [SMILES: CC(C)NC(=O)OC1CCC(c2cc(Nc3ccc4c(c3F)S(=O)(=O)N(CCO)C4)n[nH]2)C1]

The titled compound was synthesized in the procedures similar to Example 37. ¹H NMR (500 MHz, DMSO-d₆) δ 11.92 (s, 1H), 8.53 (s, 1H), 8.42-8.32 (m, 1H), 7.23 (d, J=10 Hz, 1H), 6.94 (d, J=10 Hz, 1H), 5.77 (s, 1H), 5.04-4.96 (m, 1H), 4.46 (s, 2H), 3.66 (d, J=5 Hz, 2H), 3.62-3.55 (m, 1H), 3.23 (d, J=5 Hz, 2H), 3.11-3.00 (m, 1H), 2.53-2.52 (m, 1H), 2.48-2.43 (m, 1H), 2.06-1.98 (m, 1H), 1.96-1.86 (m, 1H), 1.79-1.65 (m, 2H), 1.64-1.55 (m, 1H), 1.08-1.97 (m, 6H). LC-MS (ESI): m/z [M+H]⁺=482.2.

### Example 193: cis-3-(3-((7-fluoro-2-isopropyl-1,1-dioxido-2,3-dihydrobenzo[d]isothiazol-6-yl)amino)-1H-pyrazol-5-yl)cyclopentyl isopropylcarbamate

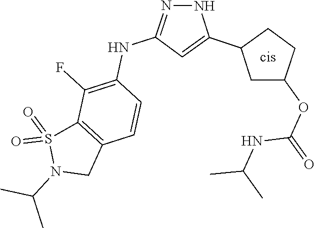 [SMILES: CC(C)NC(=O)OC1CCC(c2cc(Nc3ccc4c(c3F)S(=O)(=O)N(C(C)C)C4)n[nH]2)C1]

The titled compound was synthesized in the procedures similar to Example 63. ¹H NMR (500 MHz, DMSO-d₆) δ 11.88 (s, 1H), 8.50 (s, 1H), 8.38 (t, J=5 Hz, 1H), 7.22 (d, J=5 Hz, 1H), 6.94 (d, J=5 Hz, 1H), 5.77 (s, 1H), 5.06-4.96 (m, 1H), 4.38 (s, 2H), 4.00-3.98 (m, 1H), 3.65-3.57 (m, 1H), 3.18-3.02 (m, 1H), 2.48-2.44 (m, 1H), 2.11-1.99 (m, 1H), 1.96-1.85 (m, 1H), 1.78-1.55 (m, 3H), 1.31-1.26 (m, 6H), 1.06-1.00 (m, 6H). LC-MS (ESI): m/z [M+H]⁺=480.2.

### Example 194: cis-3-(3-((3,3-dimethyl-2,2-dioxido-1,3-dihydrobenzo[c]isothiazol-5-yl)amino)-1H-pyrazol-5-yl)cyclopentyl isopropylcarbamate

### Step 1:5-bromo-1-(4-methoxybenzyl)-3,3-dimethyl-1,3-dihydrobenzo[c]isothiazole 2,2-dioxide

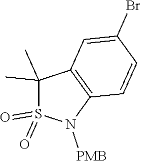

To a solution of 5-bromo-1-(4-methoxybenzyl)-1,3-dihydrobenzo[c]isothiazole 2,2-dioxide (0.25 g, 0.68 mmol) and CH₃I (213 mg, 1.5 mmol) in DMSO (10 mL), NaOH (80 mg, 2 mmol) was added at 0° C. Then the mixture was stirred at 25° C. for 1 hr. The mixture was quenched by water (10 mL) and extracted with EA (20 mL×3). The EA layer was concentrated under reduced pressure. The residue was purified by silica gel column chromatography, eluting with PE/EtOAc (10: 1~5:1) to afford the product (214 mg, 67%). LC-MS (ESI): m/z [M+Na]⁺=418.3.

### Step 2: cis-3-(3-((1-(4-methoxybenzyl)-3,3-dimethyl-2,2-dioxido-1,3-dihydrobenzo[c]isothiazol-5-yl)amino)-1H-pyrazol-5-yl)cyclopentyl isopropylcarbamate

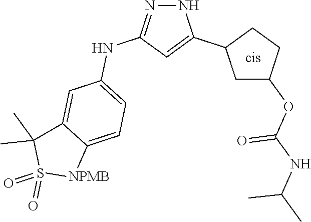

A mixture of 5-bromo-1-(4-methoxybenzyl)-3,3-dimethyl-1,3-dihydrobenzo[c]isothiazole 2,2-dioxide (50 mg, 0.13 mmol), cis-3-(5-amino-1-(tert-butyl)-1H-pyrazol-3-yl)cyclopentyl isopropylcarbamate (47.1 mg, 0.12 mmol), Brettphos Pd G3 (16.2 mg, 0.018 mmol), K₂CO₃ (74.5 mg, 0.54 mmol) in t-BuOH (10 mL) was stirred at 110° C. for 16 h under N₂ atmosphere. The mixture was concentrated under reduced pressure. The residue was purified by silica gel column chromatography to yield the product (34 mg, 33%). LC-MS (ESI): m/z [M+H]⁺=568.2.

### Step 3: cis-3-(3-((3,3-dimethyl-2,2-dioxido-1,3-dihydrobenzo[c]isothiazol-5-yl)amino)-1H-pyrazol-5-yl)cyclopentyl isopropylcarbamate

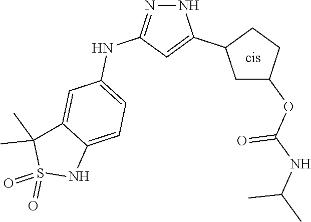 [SMILES: CC(C)NC(=O)OC1CCC(c2cc(Nc3ccc4c(c3)C(C)(C)S(=O)(=O)N4)n[nH]2)C1]

To a flask charged with cis-3-(3-((1-(4-methoxybenzyl)-3,3-dimethyl-2,2-dioxido-1,3-dihydrobenzo[c]isothiazol-5-yl)amino)-1H-pyrazol-5-yl)cyclopentyl isopropylcarbamate (34 mg, 0.06 mmol) was added TFA (1 mL) and DCM (3 mL). The result solution was stirred at room temperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (3.2 mg, 12%). ¹H NMR (500 MHz, DMSO-d₆) δ 11.69 (s, 1H), 9.55 (s, 1H), 8.23 (s, 1H), 7.38 (s, 1H), 7.15 (d, J=8.1 Hz, 1H), 6.94 (d, J=7.3 Hz, 1H), 6.77 (d, J=8.5 Hz, 1H), 5.57 (s, 1H), 4.99 (s, 1H), 3.57-3.40 (m, 1H), 3.07-2.94 (m, 1H), 2.47-2.38 (m, 1H), 2.45-2.00 (m, 1H), 1.94-1.83 (m, 1H), 1.69 (m, 2H), 1.63-1.55 (m, 1H), 1.50 (s, 6H), 1.03 (d, J=6.1 Hz, 6H). LC-MS (ESI): m/z [M+H]⁺=448.2.

### Example 195: cis-3-(3-((2,2-dioxido-1,3-dihydrobenzo[c]isothiazol-5-yl)amino)-1H-pyrazol-5-yl)cyclopentyl propylcarbamate

### Step 1:5-bromo-1-(4-methoxybenzyl)-1,3-dihydrobenzo[c]isothiazole 2,2-dioxide

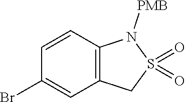

A mixture of 5-bromo-1,3-dihydrobenzo[c]isothiazole 2,2-dioxide (1 g, 3.76 mmol), 4-Methoxybenzyl bromide (1.2 g, 6 mmol) and K₂CO₃ (1.5 g, 11 mmol) in DMF (20 mL) was stirred at 20° C. for 3 h. The solution was concentrated under reduced pressure. The residue was purified by silica gel column chromatography, eluting with PE/EtOAc (3:1) to afford the product (530 mg, 41%), LC-MS (ESI): m/z [M+H]⁺=368.2.

### Step 2: cis-3-(3-((1-(4-methoxybenzyl)-2,2-dioxido-1,3-dihydrobenzo[c]isothiazol-5-yl)amino)-1H-pyrazol-5-yl)cyclopentyl propylcarbamate

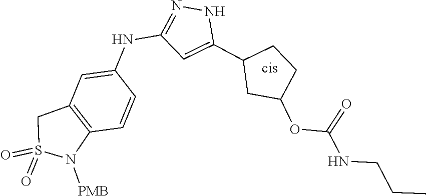

A mixture of 5-bromo-1-(4-methoxybenzyl)-1,3-dihydrobenzo[c]isothiazole 2,2-dioxide (100 mg, 0.26 mmol), cis-3-(3-amino-1H-pyrazol-5-yl)cyclopentyl propylcarbamate (65 mg, 0.26 mmol), Brettephos Pd G3 (27 mg, 0.03 mmol) and K₂CO₃ (108 mg, 0.78 mmol) in t-BuOH (15 mL) was stirred for 16 h at 110° C. in the sealed tube under N₂ atmosphere. The solution was concentrated under reduced pressure. The residue was purified by silica gel column chromatography, eluting with PE/EtOAc (1:4) to afford the crude product (80 mg, 53%), LC-MS (ESI): m/z [M+H]⁺=540.3

### Step 3: cis-3-(3-((2,2-dioxido-1,3-dihydrobenzo[c]isothiazol-5-yl)amino)-1H-pyrazol-5-yl)cyclopentyl propylcarbamate

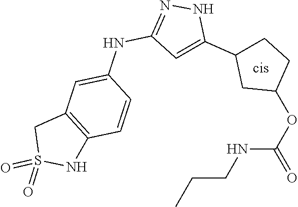 [SMILES: CCCNC(=O)OC1CCC(c2cc(Nc3ccc4c(c3)CS(=O)(=O)N4)n[nH]2)C1]

To a stirred solution of cis-3-(3-((1-(4-methoxybenzyl)-2,2-dioxido-1,3-dihydrobenzo[c]isothiazol-5-yl)amino)-1H-pyrazol-5-yl)cyclopentyl propylcarbamate (70 mg, 0.125 mmol) in DCM (5 mL) was added TFA (3 mL). The solution was stirred at 50° C. for 48 h. The solution was concentrated under reduced pressure. The residue was purified by prep HPLC (Waters SunFire C18: RD-CO-058 column, eluting with a gradient of acetonitrile/water containing 0.1% FA, 20%-55%) to afford the product in racemic form, which was further separated by Chiral Prep-HPLC to give:

Enantiomer 1 (Example 195a, 100% ee); Retention time: 4.883 min. ¹H NMR (500 MHz, DMSO-d₆) δ 11.61 (s, 1H), 9.73 (s, 1H), 8.17 (s, 1H), 7.38 (s, 1H), 7.15 (s, 1H), 7.03 (s, 1H), 6.71 (d, J=8.5 Hz, 1H), 5.57 (s, 1H), 4.99 (s, 1H), 4.43 (s, 2H), 3.08-2.95 (m, 1H), 2.94-2.85 (m, 2H), 2.48-2.42 (m, 1H), 2.04-1.85 (m, 2H), 1.78-1.55 (m, 3H), 1.42-1.34 (m, 2H), 0.82 (t, J=7.3 Hz, 3H). LC-MS (ESI): m/z [M+H]⁺=420.2.

Enantiomer 2 (Example 195b, 100% ee); Retention time: 7.152 min. ¹H NMR (500 MHz, DMSO-d₆) δ 11.61 (s, 1H), 9.73 (s, 1H), 8.17 (s, 1H), 7.38 (s, 1H), 7.15 (s, 1H), 7.03 (s, 1H), 6.71 (d, J=8.5 Hz, 1H), 5.57 (s, 1H), 4.99 (s, 1H), 4.43 (s, 2H), 3.08-2.95 (m, 1H), 2.94-2.85 (m, 2H), 2.48-2.42 (m, 1H), 2.04-1.85 (m, 2H), 1.78-1.55 (m, 3H), 1.42-1.34 (m, 2H), 0.82 (t, J=7.3 Hz, 3H). LC-MS (ESI): m/z [M+H]⁺=420.2.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=20: 80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 196: cis-3-(3-((1-methyl-2,2-dioxido-1,3-dihydrobenzo[c]isothiazol-5-yl)amino)-1H-pyrazol-5-yl)cyclopentyl spiro[2.3]hexan-4-ylcarbamate

### Step 1:5-bromo-1-methyl-1,3-dihydrobenzo[c]isothiazole 2,2-dioxide

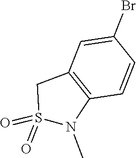

To a stirring solution of 5-bromo-1,3-dihydrobenzo[c]isothiazole 2,2-dioxide (2.5 g, 0.01 mol) in DMF (30 mL) was added K₂CO₃ (2.76 g, 0.02 mol), CH₃I (2.1 g, 0.015 mol). The solution was stirred for 3 h at RT. The solution was diluted with H₂O (50 mL), extracted with EA (50 mL×2). The organic phase was concentrated under reduced pressure. The residue was purified by silica gel column chromatography, eluting with PE/EtOAc (5:1) to afford the product (2.3 g, 94.6%), LC-MS (ESI): m/z [M+Na]⁺=283.9.

### Step 2: cis-benzyl(5-(3-hydroxycyclopentyl)-1H-pyrazol-3-yl)carbamate

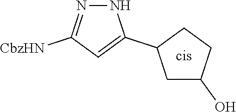 [SMILES: O=C(Nc1cc(C2CCC(O)C2)[nH]n1)OCc1ccccc1]

To a round-bottom flask charged with cis-benzyl(1-(tert-butyl)-3-(3-hydroxycyclopentyl)-1H-pyrazol-5-yl)carbamate (5 g, 13.98 mmol) was added 100 mL formic acid. The resulting mixture was stirred at 75° C. for over night. The solvent was removed under vacuum to yield crude product, which was purified by silica gel column chromatography, eluting with EA/PE (0-75%) to afford the product (3.5 g, 70%). LC-MS (ESI): m/z [M+H]⁺=302.3.

### Step 3: cis-3-(3-amino-1H-pyrazol-5-yl)cyclopentan-1-ol

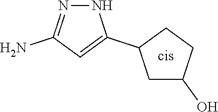 [SMILES: Nc1cc(C2CCC(O)C2)[nH]n1]

To a solution of cis-benzyl(5-(3-hydroxycyclopentyl)-1H-pyrazol-3-yl)carbamate (3.5 g, 11.6 mmol) in THF (100 mL) was added Pd/C (10%, wet, 1 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the crude product, which was directly used for the next step without further purification. LC-MS (ESI): m/z [M+H]⁺=168.3.

### Step 4: cis-5-((5-(3-hydroxycyclopentyl)-1H-pyrazol-3-yl)amino)-1-methyl-1,3-dihydrobenzo[c]isothiazole 2,2-dioxide

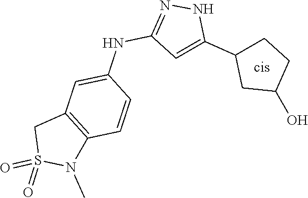 [SMILES: CN1c2ccc(Nc3cc(C4CCC(O)C4)[nH]n3)cc2CS1(=O)=O]

A mixture of 5-bromo-1-methyl-1,3-dihydrobenzo[c]isothiazole 2,2-dioxide (1 g, 3.83 mmol), cis-3-(3-amino-1H-pyrazol-5-yl)cyclopentan-1-ol (643.6 mg, 3.83 mmol), Brettphos Pd G3 (342.6 mg, 0.38 mmol), K₂CO₃ (1.59 g, 11.49 mmol) in t-BuOH (100 mL) was stirred at 110° C. for 16 h under N₂ atmosphere. The mixture was concentrated under reduced pressure. The residue was purified by silica gel column chromatography, eluting with PE/EtOAc (25%~100%) to afford the product (700 mg, 53.8%).

LC-MS (ESI): m/z [M+H]⁺=349.2

### Step 5: cis-4-nitrophenyl 3-((1-methyl-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate

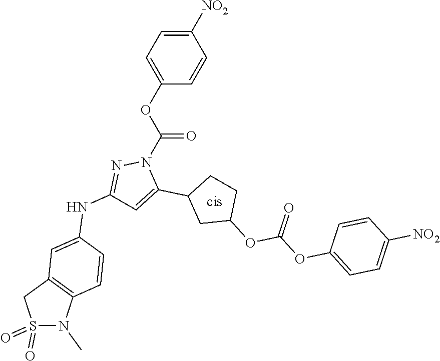 [SMILES: CN1c2ccc(Nc3cc(C4CCC(OC(=O)Oc5ccc([N+](=O)[O-])cc5)C4)n(C(=O)Oc4ccc([N+](=O)[O-])cc4)n3)cc2CS1(=O)=O]

To a stirring solution of cis-5-((5-(3-hydroxycyclopentyl)-1H-pyrazol-3-yl)amino)-1-methyl-1,3-dihydrobenzo[c]isothiazole 2,2-dioxide (1 g, 2.87 mmol) in DCM (20 mL) was added pyridine (454 mg, 5.74 mmol), DMAP (17.7 mg, 0.14 mmol) and 4-nitrophenyl carbonochloridate (865.3 mg, 4.3 mmol). The solution was stirred for 3 h at RT. The mixture was concentrated under reduced pressure. The residue was purified by silica gel column chromatography, eluting with PE/EtOAc (3:1) to afford the product (1 g, 51.5%), LC-MS (ESI): m/z [M+H]⁺=679.2.

### Step 6: cis-3-(3-((1-methyl-2,2-dioxido-1,3-dihydrobenzo[c]isothiazol-5-yl)amino)-1H-pyrazol-5-yl)cyclopentyl spiro[2.3]hexan-4-ylcarbamate

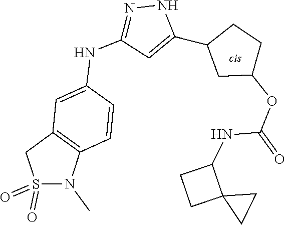 [SMILES: CN1c2ccc(Nc3cc(C4CCC(OC(=O)NC5CCC56CC6)C4)[nH]n3)cc2CS1(=O)=O]

To a stirring solution of cis-4-nitrophenyl 3-((1-methyl-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate (50 mg, 0.07 mmol) in THF (10 mL) was added DIEA (95 mg, 0.73 mmol), spiro[2.3]hexan-4-amine (33.9 mg, 0.35 mmol). The solution was stirred for 3 h at 50° C. The mixture was concentrated under reduced pressure. The residue was purified by silica gel column chromatography, eluting with PE/EtOAc (25%~100%) to afford the product (18.6 mg, 57.5%). ¹H NMR (500 MHz, DMSO-d₆) δ 11.62 (s, 1H), 8.22 (s, 1H), 7.41 (s, 1H), 7.24 (d, J=8.1 Hz, 2H), 6.78 (d, J=8.6 Hz, 1H), 5.57 (s, 1H), 5.02-4.92 (m, 1H), 4.56 (s, 2H), 4.17-4.02 (m, 1H), 3.06-2.98 (m, 1H), 2.95 (s, 3H), 2.46-2.38 (m, 1H), 2.25-2.15 (m, 1H), 2.06-1.94 (m, 3H), 1.90-1.80 (m, 1H), 1.75-1.65 (m, 3H), 1.60-1.50 (m, 1H), 0.60-0.52 (m, 1H), 0.44-0.32 (m, 2H), 0.28-0.22 (m, 1H). LC-MS (ESI): m/z [M+H]⁺=472.2.

### Example 197: cis-3-(3-((2,2-dioxido-1,3-dihydrobenzo[c]isothiazol-5-yl)amino)-1H-pyrazol-5-yl)cyclopentyl(1-methoxypropan-2-yl)carbamate

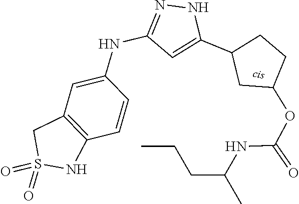 [SMILES: CCCC(C)NC(=O)OC1CCC(c2cc(Nc3ccc4c(c3)CS(=O)(=O)N4)n[nH]2)C1]

The titled compound was synthesized in the procedures similar to Example 195. ¹H NMR (500 MHz, DMSO-d₆) δ 11.63 (s, 1H), 9.74 (s, 1H), 8.21 (s, 1H), 7.41 (s, 1H), 7.26 (d, J=6.9 Hz, 1H), 6.95 (d, J=8.1 Hz, 1H), 6.78 (d, J=8.7 Hz, 1H), 5.57 (s, 1H), 4.99 (s, 1H), 4.56 (s, 2H), 3.70-3.60 (m, 1H), 3.27-3.19 (m, 4H), 3.16-3.09 (m, 1H), 3.06-2.98 (m, 1H), 2.46-2.38 (m, 1H), 2.05-1.96 (m, 1H), 1.94-1.85 (m, 1H), 1.78-1.64 (m, 2H), 1.63-1.53 (m, 1H), 1.05-0.95 (m, 3H). LC-MS (ESI): m/z [M+H]⁺=450.2.

### Example 198: cis-3-(3-((2,2-dioxido-1,3-dihydrobenzo[c]isothiazol-5-yl)amino)-1H-pyrazol-5-yl)cyclopentyl(3,3,3-trifluoropropyl)carbamate

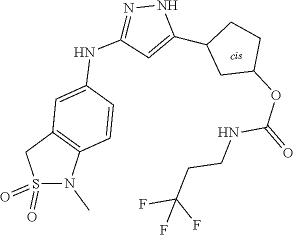 [SMILES: CN1c2ccc(Nc3cc(C4CCC(OC(=O)NCCC(F)(F)F)C4)[nH]n3)cc2CS1(=O)=O]

The titled compound was synthesized in the procedures similar to Example 195. ¹H NMR (500 MHz, DMSO-d₆) δ 11.63 (s, 1), 9.74 (s, 1H), 8.18 (s, 1H), 7.37 (s, 1H), 7.27 (t, J=5.5 Hz, 1H), 7.16 (d, J=6.9 Hz, 1H), 6.71 (d, J=8.6 Hz, 1H), 5.57 (s, 1H), 5.02-4.92 (m, 1H), 4.44 (s, 2H), 3.24-3.18 (m, 2H), 3.08-2.98 (m, 1H), 2.47-2.32 (m, 3H), 2.06-1.97 (m, 1H), 1.95-1.85 (m, 1H), 1.78-1.64 (m, 2H), 1.63-1.53 (m, 1H). LC-MS (ESI): m/z [M+H]⁺=474.2.

### Example 199: cis-3-(3-((2,2-dioxido-1H-spiro[benzo[c]isothiazole-3,l′-cyclobutan]-5-yl)amino)-1H-pyrazol-5-yl)cyclopentyl isopropylcarbamate

### Step 1:5-bromo-1-(4-methoxybenzyl)-1H-spiro[benzo[c]isothiazole-3,l′-cyclobutane] 2,2-dioxide

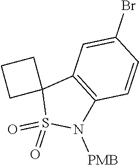

To a solution of 5-bromo-1-(4-methoxybenzyl)-1,3-dihydrobenzo[c]isothiazole 2,2-dioxide (0.25 g, 0.68 mmol) and 1,3-dibromopropane (0.183 g, 0.92 mmol) in DMF (10 mL), NaH (0.057 g, 2.37 mmol) was added at 0° C. Then the mixture was stirred at 25° C. for 1 hr. The mixture was quenched by water (10 mL) and extracted with EA (20 mL×3). The EA layer was concentrated under reduced pressure. The residue was purified by silica gel column chromatography, eluting with PE/EtOAc (9: 1~3:1) to afford the product (0.084 g, 30.3%). LC-MS (ESI): m/z [M+H]⁺=408.

### Step 2: cis-3-(3-((2,2-dioxido-1H-spiro[benzo[c]isothiazole-3,1′-cyclobutan]-5-yl)amino)-1H-pyrazol-5-yl)cyclopentyl isopropylcarbamate

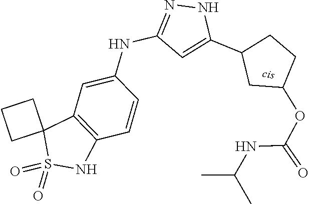 [SMILES: CC(C)NC(=O)OC1CCC(c2cc(Nc3ccc4c(c3)C3(CCC3)S(=O)(=O)N4)n[nH]2)C1]

The titled compound was synthesized in the procedures similar to Example 194. ¹H NMR (500 MHz, DMSO-d₆) δ 11.71 (s, 1H), 9.63 (s, 1H), 8.27 (s, 1H), 7.70 (s, 1H), 7.15 (d, J=8.1 Hz, 1H), 6.94 (d, J=7.5 Hz, 1H), 6.70 (d, J=8.5 Hz, 1H), 5.58 (s, 1H), 4.99 (s, 1H), 3.61-3.52 (m, 1H), 3.07-2.98 (m, 1H), 2.90-2.82 (m, 2H), 2.48-2.43 (m, 1H), 2.39-2.32 (m, 2H), 2.14-1.86 (m, 4H), 1.78-1.55 (m, 3H), 1.03 (d, J=6.3 Hz, 6H). LC-MS (ESI): m/z [M+H]⁺=460.2.

### Example 200: cis-3-(3-((2,2-dioxido-1H-spiro[benzo[c]isothiazole-3, 1′-cyclopropan]-5-yl)amino)-1H-pyrazol-5-yl)cyclopentyl isopropylcarbamate

### Step 1:5-bromo-1-(4-methoxybenzyl)-1H-spiro[benzo[c]isothiazole-3,l′-cyclopropane] 2,2-dioxide

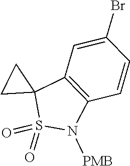

To a solution of 5-bromo-1-(4-methoxybenzyl)-1,3-dihydrobenzo[c]isothiazole 2,2-dioxide (0.25 g, 0.68 mmol) and 1,2-dibromoethane (0.160 g, 0.85 mmol) in DMF (10 mL), NaH (0.057 g, 2.37 mmol) was added at 0° C. Then the mixture was stirred at 25° C. for 12 hr. The mixture was quenched by water (10 mL) and extracted with EA (20 mL×3). The EA layer was concentrated under reduced pressure. The residue was purified by silica gel column chromatography, eluting with PE/EtOAc (9: 1~3:1) to afford the product (0.16 g, 59.9%). LC-MS (ESI): m/z [M+H]⁺=394.

### Step 2: cis-3-(3-((2,2-dioxido-1H-spiro[benzo[c]isothiazole-3,l′-cyclopropan]-5-yl)amino)-1H-pyrazol-5-yl)cyclopentyl isopropylcarbamate

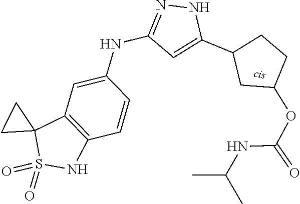 [SMILES: CC(C)NC(=O)OC1CCC(c2cc(Nc3ccc4c(c3)C3(CC3)S(=O)(=O)N4)n[nH]2)C1]

The titled compound was synthesized in the procedures similar to Example 194. ¹H NMR (500 MHz, DMSO-d₆) δ 11.65 (s, 1H), 9.95 (s, 1H), 8.16 (s, 1H), 7.12 (s, 1H), 7.01 (s, 1H), 6.94 (d, J=7.4 Hz, 1H), 6.74 (d, J=8.5 Hz, 1H), 5.55 (s, 1H), 4.98 (s, 1H), 3.61-3.53 (m, 1H), 3.04-2.97 (m, 1H), 2.46-2.41 (m, 1H), 2.02-1.85 (m, 2H), 1.74-1.53 (m, 5H), 1.47-1.42 (m, 2H), 1.03 (d, J=6.4 Hz, 6H). LC-MS (ESI): m/z [M+H]⁺=446.2.

### Example 201: cis-3-(3-((1-methyl-2,2-dioxido-1H-spiro[benzo[c]isothiazole-3,l′-cyclopropan]-5-yl)amino-1H-pyrazol-5-yl)cyclopentyl isopropylcarbamate

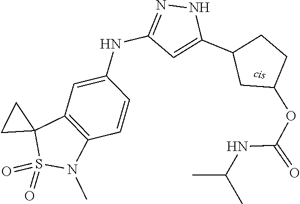 [SMILES: CC(C)NC(=O)OC1CCC(c2cc(Nc3ccc4c(c3)C3(CC3)S(=O)(=O)N4C)n[nH]2)C1]

The titled compound was synthesized in the procedures similar to Example 121. ¹H NMR (500 MHz, DMSO-d₆) δ 11.67 (s, 1H), 8.19 (s, 1H), 7.23 (d, J=7.3 Hz, 1H), 7.05 (s, 1H), 6.94 (d, J=7.1 Hz, 1H), 6.83 (d, J=8.7 Hz, 1H), 5.55 (s, 1H), 4.98 (s, 1H), 3.62-3.53 (m, 1H), 3.04 (s, 3H), 3.03-2.97 (m, 1H), 2.48-2.43 (m, 1H), 2.03-1.85 (m, 2H), 1.80 (q, J=5.2 Hz, 2H), 1.75-1.49 (m, 5H), 1.03 (d, J=6.4 Hz, 6H). LC-MS (ESI): m/z [M+H]⁺=460.2.

### Example 202: cis-3-(3-((1-isopropyl-2,2-dioxido-1,3-dihydrobenzo[c]isothiazol-5-yl)amino)-1H-pyrazol-5-yl)cyclopentyl isopropylcarbamate

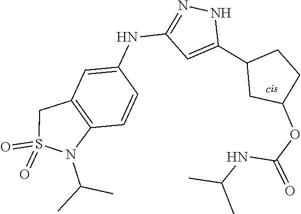 [SMILES: CC(C)NC(=O)OC1CCC(c2cc(Nc3ccc4c(c3)CS(=O)(=O)N4C(C)C)n[nH]2)C1]

The titled compound was synthesized in the procedures similar to Example 121. ¹H NMR (500 MHz, DMSO-d₆) δ 11.61 (s, 1H), 8.22 (s, 1H), 7.39 (s, 1H), 7.21 (d, J=8.6 Hz, 1H), 6.93 (dd, J=18.3, 8.2 Hz, 2H), 5.58 (s, 1H), 4.99 (s, 1H), 4.48 (s, 2H), 4.15 (dt, J=13.7, 6.9 Hz, 1H), 3.58-3.50 (m, 1H), 3.09-2.92 (m, 1H), 2.50-2.44 (m, 1H), 2.01-1.95 (m, 1H), 1.95-1.84 (m, 1H), 1.76-1.65 (m, 2H), 1.59-1.40 (m, 1H), 1.38 (d, J=6.9 Hz, 6H), 1.03 (d, J=6.4 Hz, 6H). LC-MS (ESI): m/z [M+H]⁺=462.2.

### Example 203: cis-3-(3-((1-cyclopropyl-2,2-dioxido-1,3-dihydrobenzo[c]isothiazol-5-yl)amino)-1H-pyrazol-5-yl)cyclopentyl isopropylcarbamate

### Step 1: S-(2-iodobenzyl)ethanethioate

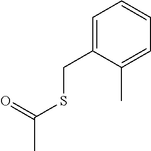

To a solution of 2-iodobenzyl bromide (30 g, 100 mmol) in DMF (110 mL) was added Potassium thioacetate (22.8 g, 200 mmol) at 0° C. The resulting solution was stirred for 2 h at rt. The reaction was then quenched by the addition of sat.aq. NH₄Cl solution. The resulting solution was extracted with 3×150 mL of EA. The resulting mixture was concentrated under vacuum and purified by combi-flash (EtOAc/PE=5%) to yield the product (27 g, 92.2%).

### Step 2: (2-iodophenyl) methanesulfonyl chloride

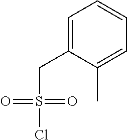

To a solution of NCS (13.8 g, 102.8 mmol) in MeCN (100 mL) and HCl (25 mL, 2M) was added the solution of S-(2-iodobenzyl)ethanethioate (7.5 g, 25.7 mmol) in MeCN (25 mL) at 0° C. The resulting solution was stirred for 30 min at 0° C. The resulting solution was extracted with 3×150 mL of EtOAc. The resulting mixture was concentrated under vacuum. The residue was the crude product (7.2 g, 90%).

### Step 3: N-cyclopropyl-1-(2-iodophenyl) methanesulfonamide

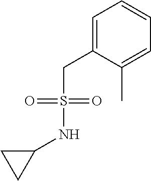

To a solution of (2-iodophenyl) methanesulfonyl chloride (7.2 g, 18.2 mmol) in THF (150 mL) was added cyclopropanamine (4.8 g, 84.7 mmol) at 0° C. The resulting solution was stirred for 1 h at rt. The resulting mixture was concentrated under vacuum. The residue was purified by combi-flash (EtOAc/PE=16%-40%) to give the product (3.3 g, 41%). LC-MS (ESI): m/z [M+H]⁺=338.2.

### Step 4:1-cyclopropyl-1,3-dihydrobenzo[c]isothiazole 2,2-dioxide

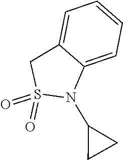 [SMILES: O=S1(=O)Cc2ccccc2N1C1CC1]

To a solution of N-cyclopropyl-1-(2-iodophenyl) methanesulfonamide (3.3 g, 9.8 mmol) in DMF (80 mL) were added CuI (262.2 mg, 1.4 mmol), sarcosine (245.6 mg, 2.8 mmol) and K₃PO₄ (4.9 g, 23 mmol). The resulting solution was stirred for 1 h at 80° C. under nitrogen atmosphere. Water (200 mL) was added, and the resulting solution was extracted with 3×50 mL of EA. The resulting mixture was concentrated under vacuum. The residue was purified by combi-flash (EA/PE=7%) to give the product (0.9 g, 64.2%). LC-MS (ESI): m/z [M+H]⁺=210.5.

### Step 5:5-bromo-1-cyclopropyl-1,3-dihydrobenzo[c]isothiazole 2,2-dioxide

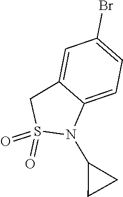 [SMILES: O=S1(=O)Cc2cc(Br)ccc2N1C1CC1]

To a stirring solution of 1-cyclopropyl-1,3-dihydrobenzo[c]isothiazole 2,2-dioxide (1 g, 4.78 mmol) in DMF (20 mL) was added NBS (781 mg, 4.39 mmol). The mixture was extracted with EtOAc (100 mL×2). The organic phase was washed with brine, dried over anhydrous sodium sulfate. The mixture was concentrated under reduced pressure. The residue was purified by silica gel column chromatography, eluting with PE/EtOAc (2:1) to afford the product (1 g, 72.9%), LC-MS (ESI): m/z [M+H]⁺=287.9.

### Step 6: cis-3-(3-((1-cyclopropyl-2,2-dioxido-1,3-dihydrobenzo[c]isothiazol-5-yl)amino)-1H-pyrazol-5-yl)cyclopentyl isopropylcarbamate

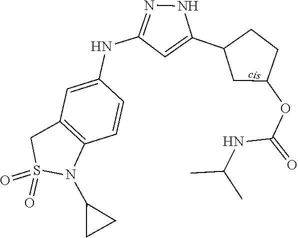 [SMILES: CC(C)NC(=O)OC1CCC(c2cc(Nc3ccc4c(c3)CS(=O)(=O)N4C3CC3)n[nH]2)C1]

The titled compound was synthesized in the procedures similar to Example 1. ¹H NMR (500 MHz, DMSO-d₆) δ 11.59 (s, 1H), 8.24 (s, 1H), 7.41 (s, 1H), 7.26 (d, J=8.5 Hz, 1H), 6.95 (d, J=8.6 Hz, 2H), 5.58 (s, 1H), 4.99 (s, 1H), 4.54 (s, 2H), 3.64-3.52 (m, 1H), 3.14-2.96 (m, 1H), 2.46-2.39 (m, 2H), 2.01-1.98 (m, 1H), 1.95-1.85 (m, 1H), 1.75-1.65 (m, 2H), 1.64-1.59 (m, 1H), 1.03 (d, J=6.3 Hz, 6H), 0.91 (dd, J=6.3, 4.2 Hz, 2H), 0.87 (d, J=2.5 Hz, 2H). LC-MS (ESI): m/z [M+H]⁺=460.2.

### Example 204: cis-3-(3-((1-(2-methoxyethyl)-2,2-dioxido-1,3-dihydrobenzo[c]isothiazol-5-yl)amino)-1H-pyrazol-5-yl)cyclopentyl isopropylcarbamate

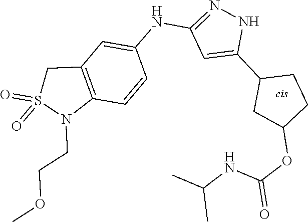 [SMILES: COCCN1c2ccc(Nc3cc(C4CCC(OC(=O)NC(C)C)C4)[nH]n3)cc2CS1(=O)=O]

The titled compound was synthesized in the procedures similar to Example 121. ¹H NMR (500 MHz, DMSO-d₆) δ 8.26 (s, 1H), 7.30 (s, 1H), 7.14 (d, J=8.6 Hz, 1H), 6.87 (d, J=7.2 Hz, 1H), 6.81 (d, J=8.7 Hz, 1H), 5.55 (s, 1H), 4.92 (s, 1H), 4.49 (s, 2H), 3.56-3.53 (m, 2H), 3.50-3.45 (m, 3H), 3.10 (s, 3H), 3.00-2.92 (m, 1H), 2.37-2.30 (m, 1H), 1.94-1.89 (m, 1H), 1.88-1.79 (m, 1H), 1.70-1.64 (m, 2H), 1.52-1.48 (m, 1H), 0.96 (d, J=6.2 Hz, 6H). LC-MS (ESI): m/z [M+H]⁺=478.2.

### Example 205: cis-3-(3-((2,2-dioxido-1-(2,2,2-trifluoroethyl)-1,3-dihydrobenzo[c]isothiazol-5-yl)amino)-1H-pyrazol-5-yl)cyclopentyl isopropylcarbamate

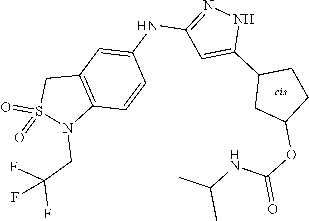 [SMILES: CC(C)NC(=O)OC1CCC(c2cc(Nc3ccc4c(c3)CS(=O)(=O)N4CC(F)(F)F)n[nH]2)C1]

The titled compound was synthesized in the procedures similar to Example 121. ¹H NMR (500 MHz, DMSO-d₆) δ 8.26 (s, 1H), 7.36 (s, 1H), 7.19 (dd, J=8.7, 1.4 Hz, 1H), 6.86 (d, J=8.7 Hz, 2H), 5.52 (s, 1H), 4.92 (s, 1H), 4.63 (s, 2H), 4.29 (q, J=9.4 Hz, 2H), 3.55-3.48 (m, 1H), 3.02-2.90 (m, 1H), 2.43 (s, 3H), 2.41-2.34 (m, 1H), 1.98-1.89 (m, 1H), 1.89-1.78 (m, 1H), 1.69-1.59 (m, 2H), 1.57-1.47 (m, 1H), 0.96 (d, J=6.3 Hz, 6H). LC-MS (ESI): m/z [M+H]⁺=502.2.

### Example 206: cis-3-(3-((1-(methyl-d3)-2,2-dioxido-1,3-dihydrobenzo[c]isothiazol-5-yl)amino)-1H-pyrazol-5-yl)cyclopentyl isopropylcarbamate

### Step 1:5-bromo-1-(methyl-d3)-1,3-dihydrobenzo[c]isothiazole 2,2-dioxide

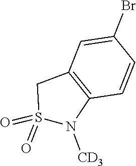 [SMILES: [2H]C([2H])([2H])N1c2ccc(Br)cc2CS1(=O)=O]

The titled compound was synthesized in the procedures similar to Example 196. LC-MS (ESI): m/z [M+Na]⁺=287.1.

### Step 2: cis-3-(3-((1-(methyl-d3)-2,2-dioxido-1,3-dihydrobenzo[c]isothiazol-5-yl)amino)-1H-pyrazol-5-yl)cyclopentyl isopropylcarbamate

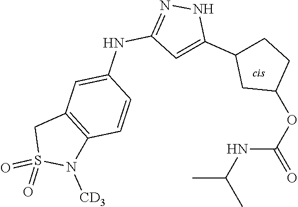 [SMILES: [2H]C([2H])([2H])N1c2ccc(Nc3cc(C4CCC(OC(=O)NC(C)C)C4)[nH]n3)cc2CS1(=O)=O]

The titled compound was synthesized in the procedures similar to Example 196. ¹H NMR (500 MHz, DMSO-d₆) δ 11.65 (s, 1H), 8.23 (s, 1H), 7.40 (s, 1H), 7.24 (s, 1H), 6.95 (d, J=7.4 Hz, 1H), 6.78 (d, J=8.6 Hz, 1H), 5.57 (s, 1H), 4.98 (s, 1H), 4.56 (s, 2H), 3.60-3.53 (m, 1H), 3.16-2.95 (m, 1H), 2.39-2.33 (m, 1H), 2.05-1.83 (m, 2H), 1.78-1.55 (m, 3H), 1.03 (d, J=6.3 Hz, 6H). LC-MS (ESI): m/z [M+H]⁺=437.2.

### Example 207: cis-3-(3-((1,3-dimethyl-2,2-dioxido-1,3-dihydrobenzo[c]isothiazol-5-yl)amino)-1H-pyrazol-5-yl)cyclopentyl isopropylcarbamate

### Step 1:5-bromo-1,3-dimethyl-1,3-dihydrobenzo[c]isothiazole 2,2-dioxide

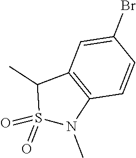

To a solution of 5-bromo-1-methyl-1,3-dihydrobenzo[c]isothiazole 2,2-dioxide (262 mg, 1 mmol) was added Cs₂CO₃ (652 mg, 2 mol), CH₃I (156 mg, 1.1 mol). The solution was stirred for 3 h at RT. The solution was diluted with H₂O (50 mL), extracted with EA (50 mL×2). The organic phase was concentrated under reduced pressure. The residue was purified by silica gel column chromatography, eluting with PE/EtOAc (5:1) to afford the product (155 mg, 56%), LC-MS (ESI): m/z [M+H]⁺=276.2.

### Step 2: cis-3-(3-((1,3-dimethyl-2,2-dioxido-1,3-dihydrobenzo[c]isothiazol-5-yl)amino)-1H-pyrazol-5-yl)cyclopentyl isopropylcarbamate

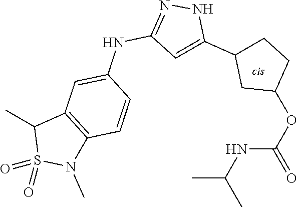 [SMILES: CC(C)NC(=O)OC1CCC(c2cc(Nc3ccc4c(c3)C(C)S(=O)(=O)N4C)n[nH]2)C1]

The titled compound was synthesized in the procedures similar to Example 34, step 5. ¹H NMR (500 MHz, DMSO-d₆) δ 11.66 (s, 1H), 8.23 (s, 1H), 7.41 (s, 1H), 7.26 (d, J=7.8 Hz, 1H), 6.94 (d, J=7.4 Hz, 1H), 6.78 (d, J=8.6 Hz, 1H), 5.57 (s, 1H), 4.98 (s, 1H), 4.55 (q, J=6.9 Hz, 1H), 3.63-3.51 (m, 1H), 3.06-2.99 (m, 1H), 2.97 (s, 3H), 2.50-2.42 (m, 1H), 2.10-1.95 (m, 1H), 1.93-1.83 (m, 1H), 1.75-1.65 (m, 2H), 1.59-1.52 (m, 1H), 1.50 (d, J=7.0 Hz, 3H), 1.03 (d, J=6.2 Hz, 6H). LC-MS (ESI): m/z [M+H]⁺=448.2.

### Example 208: cis-3-(3-((1-(1-(oxetan-3-yl)ethyl)-2,2-dioxido-1,3-dihydrobenzo[c]isothiazol-5-yl)amino)-1H-pyrazol-5-yl)cyclopentyl isopropylcarbamate

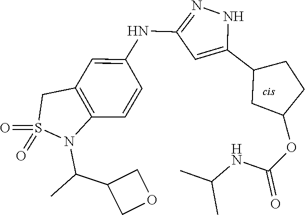 [SMILES: CC(C)NC(=O)OC1CCC(c2cc(Nc3ccc4c(c3)CS(=O)(=O)N4C(C)C3COC3)n[nH]2)C1]

The titled compound was synthesized in the procedures similar to Example 63. ¹H NMR (500 MHz, DMSO-d₆) δ 11.65 (s, 1H), 8.23 (s, 1H), 7.38 (s, 1H), 7.21 (d, J=8.3 Hz, 1H), 6.93 (d, J=8.7 Hz, 2H), 5.58 (s, 1H), 4.99 (s, 1H), 4.71-4.66 (m, 1H), 4.58 (t, J=6.9 Hz, 1H), 4.51 (s, 2H), 4.42-4.27 (m, 3H), 3.61-3.51 (m, 2H), 3.07-2.99 (m, 1H), 2.48-2.43 (m, 1H), 2.04-1.87 (m, 2H), 1.78-1.55 (m, 3H), 1.21 (d, J=6.7 Hz, 3H), 1.03 (d, J=6.3 Hz, 6H). LC-MS (ESI): m/z [M+H]⁺=504.3.

### Example 209: cis-3-(3-((1-(oxetan-3-yl)-2,2-dioxido-1,3-dihydrobenzo[c]isothiazol-5-yl)amino)-1H-pyrazol-5-yl)cyclopentyl isopropylcarbamate

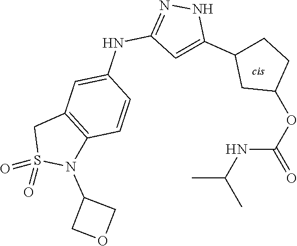 [SMILES: CC(C)NC(=O)OC1CCC(c2cc(Nc3ccc4c(c3)CS(=O)(=O)N4C3COC3)n[nH]2)C1]

The titled compound was synthesized in the procedures similar to Example 63. ¹H NMR (500 MHz, DMSO-d₆) δ 11.81 (brs, 1H), 8.32 (s, 1H), 7.44 (s, 1H), 7.22 (d, J=8.6 Hz, 1H), 6.94 (d, J=7.1 Hz, 1H), 6.76 (d, J=8.7 Hz, 1H), 5.59 (s, 1H), 5.05-4.95 (m, 3H), 4.88 (t, J=6.9 Hz, 2H), 4.85-4.77 (m, 1H), 4.65 (s, 2H), 3.65-3.50 (m, 1H), 3.10-2.95 (m, 1H), 2.48-2.40 (m, 1H), 2.05-1.96 (m, 1H), 1.96-1.85 (m, 1H), 1.80-1.64 (m, 2H), 1.63-1.52 (m, 1H), 1.03 (d, J=6.2 Hz, 6H). LC-MS (ESI): m/z [M+H]⁺=476.36.

### Example 210: cis-3-(3-((1-(oxetan-3-ylmethyl)-2,2-dioxido-1,3-dihydrobenzo[c]isothiazol-5-yl)amino)-1H-pyrazol-5-yl)cyclopentyl isopropylcarbamate

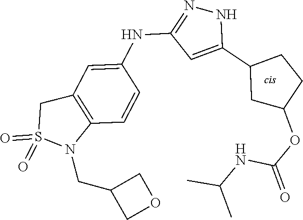 [SMILES: CC(C)NC(=O)OC1CCC(c2cc(Nc3ccc4c(c3)CS(=O)(=O)N4CC3COC3)n[nH]2)C1]

The titled compound was synthesized in the procedures similar to Example 63. ¹H NMR (500 MHz, DMSO-d₆) δ=11.65 (s, 1H), 8.22 (s, 1H), 7.39 (s, 1H), 7.23 (d, J=8.1, 1H), 6.94 (d, J=7.6, 1H), 6.83 (d, J=8.7, 1H), 5.57 (s, 1H), 4.99 (s, 1H), 4.63 (dd, J=7.7, 6.2, 2H), 4.57 (s, 2H), 4.40-4.33 (m, 2H), 3.74 (d, J=7.2, 2H), 3.57 (m, 1H), 3.42-3.34 (m, 1H), 3.08-2.96 (m, 1H), 2.48-2.41 (m, 1H), 2.00 (m, 1H), 1.95-1.83 (m, 1H), 1.69 (m, 2H), 1.65-1.52 (m, 1H), 1.03 (m, 6H). LC-MS (ESI): m/z [M+H]⁺=490.32.

### Example 211: cis-3-(3-((1-(cyclobutylmethyl)-2,2-dioxido-1,3-dihydrobenzo[c]isothiazol-5-yl)amino)-1H-pyrazol-5-yl)cyclopentyl isopropylcarbamate

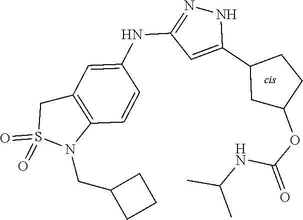 [SMILES: CC(C)NC(=O)OC1CCC(c2cc(Nc3ccc4c(c3)CS(=O)(=O)N4CC3CCC3)n[nH]2)C1]

The titled compound was synthesized in the procedures similar to Example 63. ¹H NMR (500 MHz, DMSO-d₆) δ=11.64 (s, 1H), 8.20 (s, 1H), 7.38 (s, 1H), 7.21 (d, J=8.1, 1H), 6.94 (d, J=7.2, 1H), 6.78 (d, J=8.7, 1H), 5.57 (s, 1H), 4.99 (s, 1H), 4.54 (s, 2H), 3.57 (m, 1H), 3.43 (d, J=7.0, 2H), 3.08-2.97 (m, 1H), 2.75-2.67 (m, 1H), 2.44 (m, 1H), 2.01 (m, 3H), 1.94-1.86 (m, 1H), 1.86-1.75 (m, 4H), 1.71 (m, 2H), 1.58 (m, 1H), 1.06-0.97 (m, 6H). LC-MS (ESI): m/z [M+H]⁺=488.34.

### Example 212: cis-3-(3-((1-(2-morpholinoethyl)-2,2-dioxido-1,3-dihydrobenzo[c]isothiazol-5-yl)amino)-1H-pyrazol-5-yl)cyclopentyl isopropylcarbamate

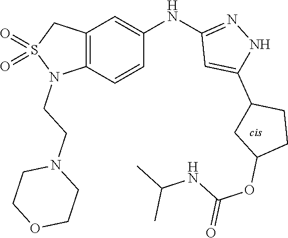 [SMILES: CC(C)NC(=O)OC1CCC(c2cc(Nc3ccc4c(c3)CS(=O)(=O)N4CCN3CCOCC3)n[nH]2)C1]

The titled compound was synthesized in the procedures similar to Example 63. ¹H NMR (500 MHz, DMSO-d₆) δ=11.75 (s, 1H), 8.22 (s, 1H), 7.39 (s, 1H), 7.23 (dd, J=8.6, 2.0, 1H), 6.94 (d, J=7.3, 1H), 6.85 (d, J=8.7, 1H), 5.57 (s, 1H), 4.99 (s, 1H), 4.55 (s, 2H), 3.63-3.48 (m, 7H), 3.09-2.96 (m, 1H), 2.56 (t, J=7.1, 2H), 2.48-2.38 (m, 5H), 2.00 (m, 1H), 1.95-1.84 (m, 1H), 1.70 (m, 2H), 1.60 (m, 1H), 1.03 (m, 6H). LC-MS (ESI): m/z [M+H]⁺=533.38.

### Example 213: cis-3-(3-((1-((1-methylazetidin-3-yl)methyl)-2,2-dioxido-1,3-dihydrobenzo[c]isothiazol-5-yl)amino)-1H-pyrazol-5-yl)cyclopentyl isopropylcarbamate

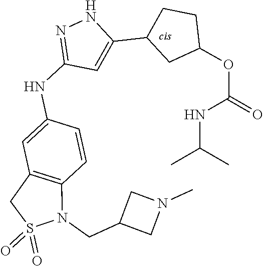 [SMILES: CC(C)NC(=O)OC1CCC(c2cc(Nc3ccc4c(c3)CS(=O)(=O)N4CC3CN(C)C3)n[nH]2)C1]

The titled compound was synthesized in the procedures similar to Example 63. ¹H NMR (500 MHz, DMSO-d₆) δ 11.86 (brs, 1H), 8.22 (d, J=2.9 Hz, 2H), 7.39 (s, 1H), 7.22 (d, J=8.6 Hz, 1H), 6.94 (d, J=7.2 Hz, 1H), 6.80 (d, J=8.7 Hz, 1H), 5.57 (s, 1H), 5.10-4.90 (m, 1H), 4.56 (s, 2H), 3.68-3.53 (m, 3H), 3.37 (s, 1H), 3.41-3.3 (m, 1H), 3.11-2.98 (m, 3H), 2.82-2.74 (m, 1H), 2.47-2.39 (m, 1H), 2.28 (s, 3H), 2.05-1.96 (m, 1H), 1.95-1.84 (m, 1H), 1.80-1.65 (m, 2H), 1.63-1.52 (m, 1H), 1.03 (d, J=6.3 Hz, 6H). LC-MS (ESI): m/z [M+H]⁺=503.33.

### Example 214: cis-3-(3-((1-((1-acetylazetidin-3-yl)methyl)-2,2-dioxido-1,3-dihydrobenzo[c]isothiazol-5-yl)amino)-1H-pyrazol-5-yl)cyclopentyl isopropylcarbamate

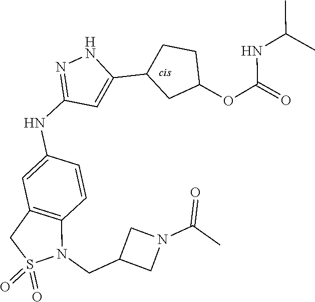 [SMILES: CC(=O)N1CC(CN2c3ccc(Nc4cc(C5CCC(OC(=O)NC(C)C)C5)[nH]n4)cc3CS2(=O)=O)C1]

The titled compound was synthesized in the procedures similar to Example 63. ¹H NMR (500 MHz, DMSO-d₆) δ 11.64 (s, 1H), 8.23 (s, 1H), 7.40 (s, 1H), 7.24 (d, J=7.2 Hz, 1H), 6.94 (d, J=7.5 Hz, 1H), 6.88 (d, J=8.7 Hz, 1H), 5.57 (s, 1H), 5.08-4.92 (m, 1H), 4.59 (s, 2H), 4.16 (t, J=8.4 Hz, 1H), 3.94-3.81 (m, 2H), 3.73-3.51 (m, 4H), 3.10-2.92 (m, 2H), 2.47-2.40 (m, 1H), 2.05-1.95 (m, 1H), 1.94-1.84 (m, 1H), 1.79-1.65 (m, 5H), 1.65-1.53 (m, 1H), 1.03 (d, J=6.4 Hz, 6H). LC-MS (ESI): m/z [M+H]⁺=531.33.

### Example 215: cis-3-(3-((1-(3-hydroxypropyl)-2,2-dioxido-1,3-dihydrobenzo[c]isothiazol-5-yl)amino-1H-pyrazol-5-yl)cyclopentyl isopropylcarbamate

### Step 1: cis-3-(3-((1-(3-((tert-butyldimethylsilyl)oxy) propyl)-2,2-dioxido-1,3-dihydrobenzo[c]isothiazol-5-yl)amino)-1H-pyrazol-5-yl)cyclopentyl isopropylcarbamate

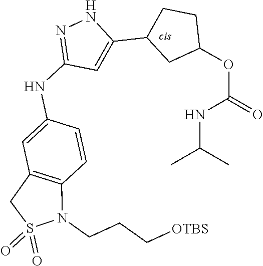 [SMILES: CC(C)NC(=O)OC1CCC(c2cc(Nc3ccc4c(c3)CS(=O)(=O)N4CCCO[Si](C)(C)C(C)(C)C)n[nH]2)C1]

The titled compound was synthesized in the procedures similar to Example 63. LC-MS (ESI): m/z [M+H]⁺=592.3.

### Step 2: cis-3-(3-((1-(3-hydroxypropyl)-2,2-dioxido-1,3-dihydrobenzo[c]isothiazol-5-yl)amino)-1H-pyrazol-5-yl)cyclopentyl isopropylcarbamate

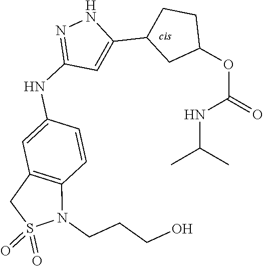 [SMILES: CC(C)NC(=O)OC1CCC(c2cc(Nc3ccc4c(c3)CS(=O)(=O)N4CCCO)n[nH]2)C1]

A solution of cis-3-(3-((1-(3-((tert-butyldimethylsilyl)oxy) propyl)-2,2-dioxido-1,3-dihydrobenzo[c]isothiazol-5-yl)amino)-1H-pyrazol-5-yl)cyclopentyl isopropylcarbamate (150 mg, 0.25 mmol) in DCM (10 mL) was added TFA (2 mL). The solution was stirred at room temperature for 2 h. The solution was concentrated under reduced pressure. The residue was dissolved in acetonitrile (10 mL), then CsF (76 mg, 0.5 mmol) was added. After stirring for 3 h at room temperature, the mixture was concentrated under reduced pressure. The residue was purified by prep HPLC (Waters SunFire C18: RD-CO-095 column, eluting with 20%-60% of acetonitrile (containing 0.1% FA) in water (containing 0.1% FA) to afford the product (10 mg, 8.4%). ¹H NMR (500 MHz, DMSO-d₆) δ 11.63 (s, 1H), 8.20 (s, 1H), 7.40 (s, 1H), 7.23 (d, J=7.1 Hz, 1H), 6.94 (d, J=7.1 Hz, 1H), 6.81 (d, J=8.7 Hz, 1H), 5.57 (s, 1H), 5.05-4.95 (m, 1H), 4.60-4.55 (m, 1H), 4.54 (s, 2H), 3.65-3.47 (m, 5H), 3.08-2.96 (m, 1H), 2.48-2.40 (m, 1H), 2.05-1.96 (m, 1H), 1.95-1.86 (m, 1H), 1.84-1.76 (m, 2H), 1.76-1.65 (m, 2H), 1.64-1.52 (m, 1H), 1.03 (d, J=6.3 Hz, 6H). LC-MS (ESI): m/z [M+H]⁺=478.37.

### Example 216: cis-3-(3-((1-(2-hydroxy-2-methylpropyl)-2,2-dioxido-1,3-dihydrobenzo[c]isothiazol-5-yl)amino)-1H-pyrazol-5-yl)cyclopentyl isopropylcarbamate

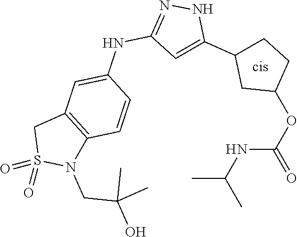 [SMILES: CC(C)NC(=O)OC1CCC(c2cc(Nc3ccc4c(c3)CS(=O)(=O)N4CC(C)(C)O)n[nH]2)C1]

The titled compound was synthesized in the procedures similar to Example 63. ¹H NMR (500 MHz, DMSO-d₆) δ 11.63 (s, 1H), 8.19 (s, 1H), 7.38 (brs, 1H), 7.17 (d, J=5 Hz, 1H), 7.02 (d, J=10 Hz, 1H), 6.97-6.91 (m, 1H), 5.57 (s, 1H), 5.02-4.95 (m, 1H), 4.62 (s, 1H), 4.55 (s, 2H), 3.62-3.53 (m, 1H), 3.28 (s, 2H), 3.07-2.97 (m, 1H), 2.47-2.40 (m, 1H), 2.03-1.96 (m, 1H), 1.94-1.84 (m, 1H), 1.78-1.65 (m, 2H), 1.63-1.53 (m, 1H), 1.20 (s, 6H), 1.07-0.97 (m, 6H). LC-MS (ESI): m/z [M+H]⁺=492.2.

### Example 217: cis-3-(3-((2,2-dioxido-1-(2-(tetrahydro-2H-pyran-4-yl)ethyl)-1,3-dihydrobenzo[c]isothiazol-5-yl)amino)-1H-pyrazol-5-yl)cyclopentyl isopropylcarbamate

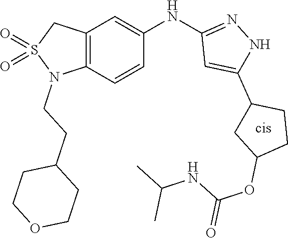 [SMILES: CC(C)NC(=O)OC1CCC(c2cc(Nc3ccc4c(c3)CS(=O)(=O)N4CCC3CCOCC3)n[nH]2)C1]

The titled compound was synthesized in the procedures similar to Example 63. ¹H NMR (500 MHz, DMSO-d₆) δ 11.63 (s, 1H), 8.21 (s, 1H), 7.40 (s, 1H), 7.23 (s, 1H), 6.94 (d, J=6.6 Hz, 1H), 6.79 (d, J=8.7 Hz, 1H), 5.57 (s, 1H), 4.99 (s, 1H), 4.54 (s, 2H), 3.83 (dd, J=11.2, 3.7 Hz, 2H), 3.62-3.53 (m, 1H), 3.50-3.45 (m, 2H), 3.29-3.23 (m, 2H), 3.08-2.98 (m, 1H), 2.48-2.43 (m, 1H), 2.03-1.86 (m, 2H), 1.74-1.55 (m, 8H), 1.25-1.14 (m, 2H), 1.03 (d, J=6.3 Hz, 6H). LC-MS (ESI): m/z [M+H]⁺=532.3.

### Example 218: cis-3-(3-((2,2-dioxido-1-(1-(tetrahydrofuran-3-yl)ethyl)-1,3-dihydrobenzo[c]isothiazol-5-yl)amino)-1H-pyrazol-5-yl)cyclopentyl isopropylcarbamate

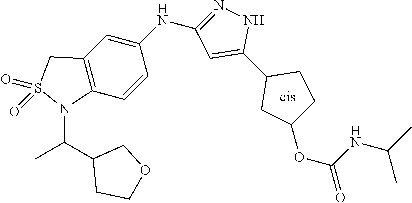 [SMILES: CC(C)NC(=O)OC1CCC(c2cc(Nc3ccc4c(c3)CS(=O)(=O)N4C(C)C3CCOC3)n[nH]2)C1]

The titled compound was synthesized in the procedures similar to Example 63. ¹H NMR (500 MHz, DMSO-d₆) δ 11.64 (s, 1H), 8.23 (s, 1H), 7.40 (s, 1H), 7.20 (s, 1H), 6.97 (dd, J=19.0, 10.0 Hz, 2H), 5.58 (s, 1H), 4.99 (s, 1H), 4.60-4.49 (m, 2H), 3.85-3.54 (m, 5H), 3.46 (dd, J=16.7, 7.9 Hz, 1H), 3.08-2.98 (m, 1H), 2.90-2.81 (m, 1H), 2.47-2.42 (m, 1H), 2.08-1.86 (m, 3H), 1.75-1.57 (m, 4H), 1.29 (dd, J=29.7, 6.7 Hz, 3H), 1.03 (d, J=6.4 Hz, 6H). LC-MS (ESI): m/z [M+H]⁺=518.3.

### Example 219: cis-3-(3-((1-(2-hydroxyethyl)-2,2-dioxido-1,3-dihydrobenzo[c]isothiazol-5-yl)amino)-1H-pyrazol-5-yl)cyclopentyl isopropylcarbamate

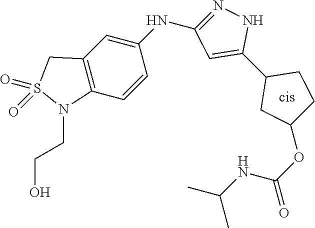 [SMILES: CC(C)NC(=O)OC1CCC(c2cc(Nc3ccc4c(c3)CS(=O)(=O)N4CCO)n[nH]2)C1]

The titled compound was synthesized in the procedures similar to Example 215 in racemic form, which was further separated by Chiral Prep-HPLC to give:

Enantiomer 1 (Example 219a, 100% ee); Retention time: 6.02 min. ¹H NMR (500 MHz, DMSO-d₆) δ 11.62 (s, 1H), 8.20 (s, 1H), 7.40 (s, 1H), 7.21 (s, 1H), 6.95 (d, J=7.6 Hz, 1H), 6.86 (d, J=8.6 Hz, 1H), 5.57 (s, 1H), 4.99 (s, 1H), 4.92 (t, J=5.6 Hz, 1H), 4.54 (s, 2H), 3.68-3.54 (m, 3H), 3.49 (t, J=6.3 Hz, 2H), 3.10-2.95 (m, 1H), 2.50-2.44 (m, 1H), 2.00-1.95 (m, 1H), 1.93-1.83 (m, 1H), 1.71-1.65 (m, 2H), 1.58-1.48 (m, 1H), 1.03 (d, J=6.4 Hz, 6H). LC-MS (ESI): m/z [M+H]⁺=464.2

Enantiomer 2 (Example 219b, 100% ee); Retention time: 8.51 min. ¹H NMR (500 MHz, DMSO-d₆) δ 11.62 (s, 1H), 8.20 (s, 1H), 7.40 (s, 1H), 7.21 (s, 1H), 6.95 (d, J=7.6 Hz, 1H), 6.86 (d, J=8.6 Hz, 1H), 5.57 (s, 1H), 4.99 (s, 1H), 4.92 (t, J=5.6 Hz, 1H), 4.54 (s, 2H), 3.68-3.54 (m, 3H), 3.49 (t, J=6.3 Hz, 2H), 3.10-2.95 (m, 1H), 2.50-2.44 (m, 1H), 2.00-1.95 (m, 1H), 1.93-1.83 (m, 1H), 1.71-1.65 (m, 2H), 1.58-1.48 (m, 1H), 1.03 (d, J=6.4 Hz, 6H). LC-MS (ESI): m/z [M+H]⁺=464.2.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for Hexane and B for EtOH (0.2% 2M NH₃ MeOH); Gradient: Mobile Phase A: Mobile Phase B=20: 80 (v/v); Flow Rate: 18 mL/min, Wave Length: UV 270 nm and 320 nm, Prep-HPLC Equipment: Prep-HPLC-Gilson, Back pressure: 100 bar; Column temperature: RT.

### Example 220: (1R, 3S)-3-(3-((1-methyl-2,2-dioxido-1,3-dihydrobenzo[c]isothiazol-5-yl)amino)-1H-pyrazol-5-yl)cyclopentyl isopropylcarbamate and (1S, 3R)-3-(3-((1-methyl-2,2-dioxido-1,3-dihydrobenzo[c]isothiazol-5-yl)amino)-1H-pyrazol-5-yl)cyclopentyl isopropylcarbamate

### Step 1:5-bromo-1-methyl-1,3-dihydrobenzo[c]isothiazole 2,2-dioxide

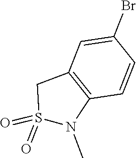

To a stirring solution of 5-bromo-1,3-dihydrobenzo[c]isothiazole 2,2-dioxide (2.5 g, 0.01 mol) in DMF (30 mL) was added K₂CO₃ (2.76 g, 0.02 mol), CH₃I (2.1 g, 0.015 mol). The solution was stirred for 3 h at RT. The solution was diluted with H₂O (50 mL), extracted with EA (50 mL×2). The organic phase was concentrated under reduced pressure. The residue was purified by silica gel column chromatography, eluting with PE/EtOAc (5:1) to afford the product (2.3 g, 94.6%), LC-MS (ESI): m/z [M+Na]⁺=283.9.

### Step 2: cis-3-(3-((1-methyl-2,2-dioxido-1,3-dihydrobenzo[c]isothiazol-5-yl)amino)-1H-pyrazol-5-yl)cyclopentyl isopropylcarbamate

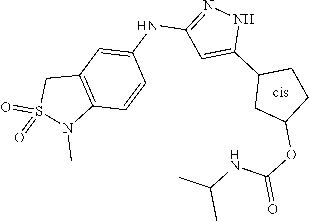 [SMILES: CC(C)NC(=O)OC1CCC(c2cc(Nc3ccc4c(c3)CS(=O)(=O)N4C)n[nH]2)C1]

A mixture of 5-bromo-1-methyl-1,3-dihydrobenzo[c]isothiazole 2,2-dioxide (150 mg, 0.57 mmol), cis-3-(3-amino-1H-pyrazol-5-yl)cyclopentyl isopropylcarbamate (144.2 mg, 0.57 mmol), Brettphos Pd G3 (51.3 mg, 0.057 mmol), K₂CO₃ (235.9 mg, 1.71 mmol) in t-BuOH (20 mL) was stirred at 110° C. for 16 h under N₂ atmosphere. The mixture was concentrated under reduced pressure. The residue was purified by silica gel column chromatography, eluting with PE/EtOAc (20%~100%) to afford the product. The residue was purified by prep HPLC (Waters XSelect C18: RD-CO-094 column, eluting with a gradient of acetonitrile/water containing 0.1% FA, 27%-52%) to afford the product in racemic form, which was further separated by Chiral Prep-HPLC to give:

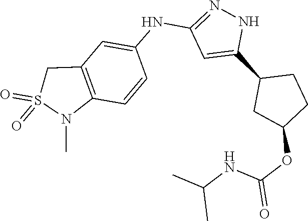 [SMILES: CC(C)NC(=O)O[C@@H]1CC[C@H](c2cc(Nc3ccc4c(c3)CS(=O)(=O)N4C)n[nH]2)C1]

Enantiomer 1 ((1R, 3S)-3-(3-((1-methyl-2,2-dioxido-1,3-dihydrobenzo[c]isothiazol-5-yl)amino)-1H-pyrazol-5-yl)cyclopentyl isopropylcarbamate, Example 220a, 100% ee); Retention time: 9.891 min. ¹H NMR (500 MHz, DMSO-d₆) δ 11.69 (s, 1H), 8.38 (s, 1H), 7.38 (s, 1H), 7.24 (d, J=8.8 Hz, 1H), 6.95 (d, J=7.5 Hz, 1H), 6.81 (d, J=8.6 Hz, 1H), 5.63 (s, 1H), 4.99 (s, 1H), 4.58 (s, 2H), 3.65-3.58 (m, 1H), 3.10-3.00 (m, 1H), 2.96 (s, 3H), 2.48-2.43 (m, 1H), 2.05-1.97 (m, 1H), 1.96-1.84 (m, 1H), 1.80-1.72 (m, 2H), 1.65-1.58 (m, 1H), 1.03 (d, J=6.0 Hz, 6H). LC-MS (ESI): m/z [M+H]⁺=434.2.

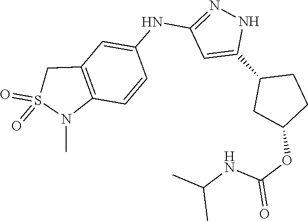 [SMILES: CC(C)NC(=O)O[C@H]1CC[C@@H](c2cc(Nc3ccc4c(c3)CS(=O)(=O)N4C)n[nH]2)C1]

Enantiomer 2 ((1S, 3R)-3-(3-((1-methyl-2,2-dioxido-1,3-dihydrobenzo[c]isothiazol-5-yl)amino)-1H-pyrazol-5-yl)cyclopentyl isopropylcarbamate, Example 220b, 100% ee); Retention time: 13.338 min. ¹H NMR (500 MHz, DMSO-d₆) δ 11.69 (s, 1H), 8.38 (s, 1H), 7.38 (s, 1H), 7.24 (d, J=8.8 Hz, 1H), 6.95 (d, J=7.5 Hz, 1H), 6.81 (d, J=8.6 Hz, 1H), 5.63 (s, 1H), 4.99 (s, 1H), 4.58 (s, 2H), 3.65-3.58 (m, 1H), 3.10-3.00 (m, 1H), 2.96 (s, 3H), 2.48-2.43 (m, 1H), 2.05-1.97 (m, 1H), 1.96-1.84 (m, 1H), 1.80-1.72 (m, 2H), 1.65-1.58 (m, 1H), 1.03 (d, J=6.0 Hz, 6H). LC-MS (ESI): m/z [M+H]⁺=434.2.

Chiral analytical method: Column: CHIRALPAK IE 4.6 mm×250 mm, 5 μm; Mobile phase: A for hexane and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for hexane and B for EtOH (0.2% 2M NH₃ MeOH); Gradient: Mobile Phase A: Mobile Phase B=20: 80 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 221: cis-3-(3-((1-methyl-2,2-dioxido-1,3-dihydrobenzo[c]isothiazol-5-yl)amino)-1H-pyrazol-5-yl)cyclopentyl propylcarbamate

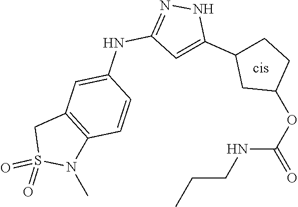 [SMILES: CCCNC(=O)OC1CCC(c2cc(Nc3ccc4c(c3)CS(=O)(=O)N4C)n[nH]2)C1]

A mixture of 5-bromo-1-methyl-1,3-dihydrobenzo[c]isothiazole 2,2-dioxide (50 mg, 0.19 mmol), cis-3-(3-amino-1H-pyrazol-5-yl)cyclopentyl propylcarbamate (47.1 mg, 0.19 mmol), Brettphos Pd G3 (16.2 mg, 0.018 mmol) and K₂CO₃ (74.5 mg, 0.57 mmol) in t-BuOH (10 mL) was stirred at 110° C. for 16 h under N₂ atmosphere. The mixture was concentrated under reduced pressure. The residue was purified by silica gel column chromatography. The residue was purified by prep HPLC (Waters XSelect C18: RD-CO-094 column, eluting with a gradient of acetonitrile/water containing 0.1% FA, 20%-40%) to afford the product (54.9 mg, 63.8%). ¹H NMR (500 MHz, DMSO-d₆) δ 11.66 (s, 1H), 8.21 (s, 1H), 7.40 (s, 1H), 7.25 (d, J=8.5 Hz, 1H), 7.04 (s, 1H), 6.78 (d, J=8.7 Hz, 1H), 5.57 (s, 1H), 4.98 (s, 1H), 4.56 (s, 2H), 3.06-2.97 (m, 2H), 2.95 (s, 3H), 2.93-2.88 (m, 2H), 2.43-2.41 (m, 1H), 2.05-1.96 (m, 1H), 1.95-1.85 (m, 1H), 1.75-1.69 (m, 2H), 1.59-1.48 (m, 1H), 1.42-1.34 (m, 2H), 0.82 (t, 3H). LC-MS (ESI): m/z [M+H]⁺=434.2.

### Example 222: cis-3-(3-((1-methyl-2,2-dioxido-1,3-dihydrobenzo[c]isothiazol-5-yl)amino)-1H-pyrazol-5-yl)cyclopentyl((S)-sec-butyl)carbamate

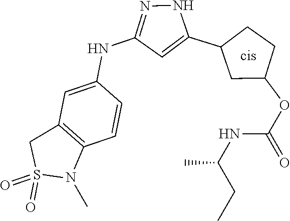 [SMILES: CC[C@H](C)NC(=O)OC1CCC(c2cc(Nc3ccc4c(c3)CS(=O)(=O)N4C)n[nH]2)C1]

The titled compound was synthesized in the procedures similar to Example 121. ¹H NMR (500 MHz, DMSO-d₆)) δ 11.62 (s, 1H), 8.20 (s, 1H), 7.41 (s, 1H), 7.26 (d, J=8.2 Hz, 1H), 6.87 (d, J=7.8 Hz, 1H), 6.78 (d, J=8.6 Hz, 1H), 5.57 (s, 1H), 4.99 (s, 1H), 4.56 (s, 2H), 3.45-3.35 (m, 1H), 3.10-3.02 (m, 1H), 2.95 (s, 3H), 2.44-2.39 (m, 1H), 2.05-1.98 (m, 1H), 1.94-1.87 (m, 1H), 1.71-1.65 (m, 2H), 1.58-1.48 (m, 1H), 1.40-1.30 (m, 2H), 1.00 (d, J=6.4 Hz, 3H), 0.80 (dd, J=13.5, 6.3 Hz, 3H). LC-MS (ESI): m/z [M+H]⁺=448.2.

### Example 223: cis-3-(3-((1-methyl-2,2-dioxido-1,3-dihydrobenzo[c]isothiazol-5-yl)amino)-1H-pyrazol-5-yl)cyclopentyl(1-methylcyclopropyl)carbamate

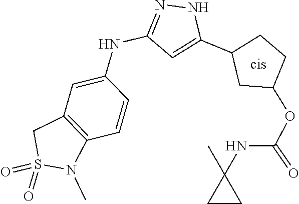 [SMILES: CN1c2ccc(Nc3cc(C4CCC(OC(=O)NC5(C)CC5)C4)[nH]n3)cc2CS1(=O)=O]

The titled compound was synthesized in the procedures similar to Example 121. ¹H NMR (500 MHz, DMSO-d₆)) δ 11.62 (s, 1H), 8.20 (s, 1H), 7.40 (s, 1H), 7.33 (s, 1H), 7.24 (s, 1H), 6.78 (d, J=8.7 Hz, 1H), 5.57 (s, 1H), 4.98 (s, 1H), 4.56 (s, 2H), 3.05-2.98 (m, 1H), 2.95 (s, 3H), 2.44-2.40 (m, 1H), 2.05-1.98 (m, 1H), 1.94-1.87 (m, 1H), 1.71-1.65 (m, 2H), 1.58-1.48 (m, 1H), 1.23 (s, 3H), 0.59 (m, 2H), 0.47 (m, 2H). LC-MS (ESI): m/z [M+H]⁺=446.2.

### Example 224: cis-3-(3-((1-methyl-2,2-dioxido-1,3-dihydrobenzo[c]isothiazol-5-yl)amino)-1H-pyrazol-5-yl)cyclopentyl spiro[3.3]heptan-2-ylcarbamate

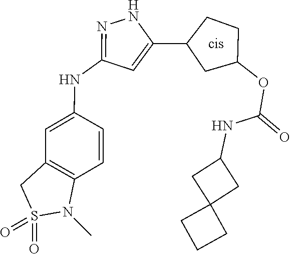 [SMILES: CN1c2ccc(Nc3cc(C4CCC(OC(=O)NC5CC6(CCC6)C5)C4)[nH]n3)cc2CS1(=O)=O]

The titled compound was synthesized in the procedures similar to Example 196. ¹H NMR (500 MHz, DMSO-d₆) δ 11.86 (s, 1H), 8.26 (s, 1H), 7.39 (s, 1H), 7.32-7.18 (m, 2H), 6.79 (d, J=8.7 Hz, 1H), 5.58 (s, 1H), 4.96 (d, J=3.0 Hz, 1H), 4.57 (s, 2H), 3.85-3.75 (m, 1H), 3.07-2.98 (m, 1H), 2.95 (s, 3H), 2.46-2.37 (m, 1H), 2.25-2.15 (m, 2H), 2.05-1.95 (m, 3H), 1.92-1.80 (m, 5H), 1.77-1.64 (m, 4H), 1.61-1.53 (m, 1H). LC-MS (ESI): m/z [M+H]⁺=486.2.

### Example 225: cis-3-(3-((1-methyl-2,2-dioxido-1,3-dihydrobenzo[c]isothiazol-5-yl)amino)-1H-pyrazol-5-yl)cyclopentyl methylcarbamate

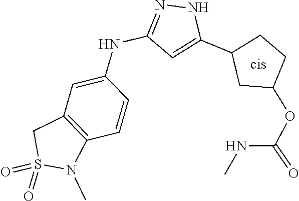 [SMILES: CNC(=O)OC1CCC(c2cc(Nc3ccc4c(c3)CS(=O)(=O)N4C)n[nH]2)C1]

The titled compound was synthesized in the procedures similar to Example 196. ¹H NMR (500 MHz, DMSO-d₆) δ 11.80 (s, 1H), 8.25 (s, 1H), 8.13 (s, 1H), 7.39 (s, 1H), 7.24 (dd, J=8.6, 2.0 Hz, 1H), 6.91 (d, J=4.4 Hz, 1H), 6.79 (d, J=8.7 Hz, 1H), 5.58 (s, 1H), 4.99 (d, J=5.4 Hz, 1H), 4.57 (s, 2H), 3.10-3.00 (m, 1H), 2.95 (s, 3H), 2.55 (d, J=4.6 Hz, 3H), 2.48-2.38 (m, 1H), 2.05-1.97 (m, 1H), 1.95-1.81 (m, 1H), 1.78-1.64 (m, 2H), 1.63-1.53 (m, 1H). LC-MS (ESI): m/z [M+H]⁺=406.2.

### Example 226: cis-3-(3-((1-methyl-2,2-dioxido-1,3-dihydrobenzo[c]isothiazol-5-yl)amino)-1H-pyrazol-5-yl)cyclopentyl(1-methoxypropan-2-yl)carbamate

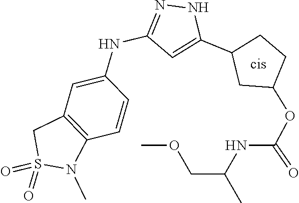 [SMILES: COCC(C)NC(=O)OC1CCC(c2cc(Nc3ccc4c(c3)CS(=O)(=O)N4C)n[nH]2)C1]

The titled compound was synthesized in the procedures similar to Example 196. ¹H NMR (500 MHz, DMSO-d₆) δ 11.63 (s, 1H), 8.21 (s, 1H), 7.41 (s, 1H), 7.26 (d, J=6.9 Hz, 1H), 6.95 (d, J=8.1 Hz, 1H), 6.78 (d, J=8.7 Hz, 1H), 5.57 (s, 1H), 4.99 (s, 1H), 4.56 (s, 2H), 3.70-3.60 (m, 1H), 3.27-3.19 (m, 4H), 3.16-3.09 (m, 1H), 3.06-2.98 (m, 1H), 2.95 (s, 3H), 2.46-2.38 (m, 1H), 2.05-1.96 (m, 1H), 1.94-1.85 (m, 1H), 1.78-1.64 (m, 2H), 1.63-1.53 (m, 1H), 1.05-0.95 (m, 3H). LC-MS (ESI): m/z [M+H]⁺=464.2.

### Example 227: cis-3-(3-((1-methyl-2,2-dioxido-1,3-dihydrobenzo[c]isothiazol-5-yl)amino)-1H-pyrazol-5-yl)cyclopentyl cyclobutylcarbamate

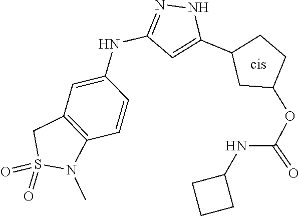 [SMILES: CN1c2ccc(Nc3cc(C4CCC(OC(=O)NC5CCC5)C4)[nH]n3)cc2CS1(=O)=O]

The titled compound was synthesized in the procedures similar to Example 196 in a racemic form was further separated by Chiral Prep-HPLC to give:

Enantiomer 1 (Example 227a, 100% ee); Retention time: 11.07 min. ¹H NMR (500 MHz, DMSO-d₆) δ 11.63 (s, 1H), 8.22 (s, 1H), 7.41 (s, 1H), 7.36 (d, J=8.1 Hz, 1H), 7.25 (s, 1H), 6.78 (d, J=8.7 Hz, 1H), 5.57 (s, 1H), 5.02-4.92 (m, 1H), 4.56 (s, 2H), 4.00-3.98 (m, 1H), 3.07-2.98 (m, 1H), 2.95 (s, 3H), 2.47-2.38 (m, 1H), 2.17-2.06 (m, 2H), 2.04-1.95 (m, 1H), 1.93-1.79 (m, 3H), 1.78-1.64 (m, 2H), 1.63-1.53 (m, 1H). LC-MS (ESI): m/z [M+H]⁺=446.2.

Enantiomer 2 (Example 227b, 98.73% ee); Retention time: 13.58 min. ¹H NMR (500 MHz, DMSO-d₆) δ 11.63 (s, 1H), 8.22 (s, 1H), 7.41 (s, 1H), 7.36 (d, J=8.1 Hz, 1H), 7.25 (s, 1H), 6.78 (d, J=8.7 Hz, 1H), 5.57 (s, 1H), 5.02-4.92 (m, 1H), 4.56 (s, 2H), 4.00-3.98 (m, 1H), 3.07-2.98 (m, 1H), 2.95 (s, 3H), 2.47-2.38 (m, 1H), 2.17-2.06 (m, 2H), 2.04-1.95 (m, 1H), 1.93-1.79 (m, 3H), 1.78-1.64 (m, 2H), 1.63-1.53 (m, 1H). LC-MS (ESI): m/z [M+H]⁺=446.2.

Chiral analytical method: Column: CHIRALPAK IE 4.6 mm×250 mm, 5 μm; Mobile phase: A for Hexane and B for EtOH (0.1% 2M NH₃ MeOH) (%); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.1% 2M NH₃ MeOH) (%); Gradient: Mobile Phase A: Mobile Phase B=20: 80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 228: cis-3-(3-((1-methyl-2,2-dioxido-1,3-dihydrobenzo[c]isothiazol-5-yl)amino)-1H-pyrazol-5-yl)cyclopentyl(1-(methoxymethyl)cyclopropyl)carbamate

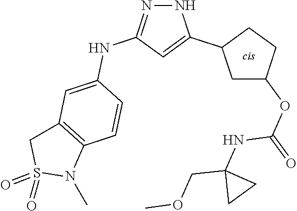 [SMILES: COCC1(NC(=O)OC2CCC(c3cc(Nc4ccc5c(c4)CS(=O)(=O)N5C)n[nH]3)C2)CC1]

The titled compound was synthesized in the procedures similar to Example 196. ¹H NMR (500 MHz, DMSO-d₆) δ 11.86 (s, 1H), 8.39 (s, 1H), 7.41 (s, 1H), 7.37 (s, 1H), 7.24 (dd, J=8.6, 2.0 Hz, 1H), 6.81 (d, J=8.6 Hz, 1H), 5.63 (s, 1H), 4.98 (s, 1H), 4.58 (s, 2H), 3.23 (s, 3H), 3.10-3.00 (m, 1H), 2.96 (s, 3H), 2.48-2.44 (m, 1H), 2.07-1.97 (m, 1H), 1.97-1.86 (m, 1H), 1.79-1.66 (m, 2H), 1.63-1.53 (m, 1H). LC-MS (ESI): m/z [M+H]⁺=476.2.

### Example 229: cis-3-(3-((1-methyl-2,2-dioxido-1,3-dihydrobenzo[c]isothiazol-5-yl)amino)-1H-pyrazol-5-yl)cyclopentyl(4,4,4-trifluorobutan-2-yl)carbamate

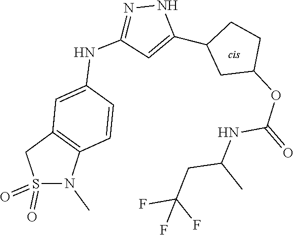 [SMILES: CC(CC(F)(F)F)NC(=O)OC1CCC(c2cc(Nc3ccc4c(c3)CS(=O)(=O)N4C)n[nH]2)C1]

The titled compound was synthesized in the procedures similar to Example 196. ¹H NMR (500 MHz, DMSO-d₆) δ 11.63 (s, 1H), 8.18 (s, 1H), 7.37 (s, 1H), 7.27 (t, J=5.5 Hz, 1H), 7.16 (d, J=6.9 Hz, 1H), 6.71 (d, J=8.6 Hz, 1H), 5.57 (s, 1H), 5.02-4.92 (m, 1H), 4.44 (s, 2H), 3.24-3.18 (m, 2H), 3.08-2.98 (m, 1H), 2.95 (s, 3H), 47-2.32 (m, 3H), 2.06-1.97 (m, 1H), 1.95-1.85 (m, 1H), 1.78-1.64 (m, 2H), 1.63-1.53 (m, 1H). LC-MS (ESI): m/z [M+H]⁺=502.2.

### Example 230: cis-3-(3-((1-methyl-2,2-dioxido-1,3-dihydrobenzo[c]isothiazol-5-yl)amino)-1H-pyrazol-5-yl)cyclopentyl(3,3,3-trifluoropropyl)carbamate

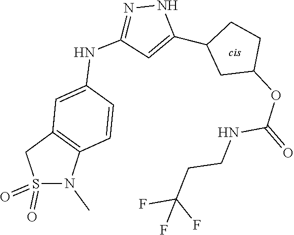 [SMILES: CN1c2ccc(Nc3cc(C4CCC(OC(=O)NCCC(F)(F)F)C4)[nH]n3)cc2CS1(=O)=O]

The titled compound was synthesized in the procedures similar to Example 196. ¹H NMR (500 MHz, DMSO-d₆) δ 11.90 (s, 1H), 8.34 (s, 1H), 7.38 (s, 1H), 7.30-7.18 (m, 2H), 6.80 (d, J=8.7 Hz, 1H), 5.62 (s, 1H), 5.02-4.92 (m, 1H), 4.57 (s, 3H), 3.25-3.15 (m, 2H), 3.12-2.99 (m, 1H), 2.95 (s, 3H), 2.46-2.32 (m, 3H), 2.07-1.97 (m, 1H), 1.97-1.86 (m, 1H), 1.79-1.66 (m, 2H), 1.63-1.53 (m, 1H). LC-MS (ESI): m/z [M+H]⁺=488.2.

### Example 231: cis-3-(3-((1-methyl-2,2-dioxido-1,3-dihydrobenzo[c]isothiazol-5-yl)amino)-1H-pyrazol-5-yl)cyclopentyl bicyclo[1.1.1]pentan-1-ylcarbamate

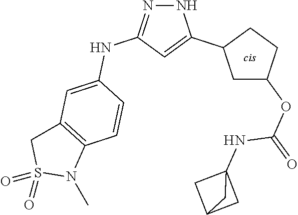 [SMILES: CN1c2ccc(Nc3cc(C4CCC(OC(=O)NC56CC(C5)C6)C4)[nH]n3)cc2CS1(=O)=O]

The titled compound was synthesized in the procedures similar to Example 196. ¹H NMR (500 MHz, DMSO-d₆) δ 11.63 (s, 1H), 8.22 (s, 1H), 7.76 (s, 1H), 7.42 (s, 1H), 7.26 (d, J=8.3 Hz, 1H), 6.78 (d, J=8.7 Hz, 1H), 5.57 (s, 1H), 4.98 (s, 1H), 4.56 (s, 2H), 3.07-2.98 (m, 1H), 2.95 (s, 3H), 2.89 (s, 1H), 2.73 (s, 1H), 2.46-2.40 (m, 1H), 2.38-2.30 (m, 1H), 2.05-1.98 (m, 1H), 1.90 (s, 7H), 1.78-1.66 (m, 2H), 1.60-1.52 (m, 1H). LC-MS (ESI): m/z [M+H]⁺=458.2.

### Example 232: cis-3-(3-((2,2-dioxido-1,3-dihydrobenzo[c]isothiazol-5-yl)amino)-1H-pyrazol-5-yl)cyclopentyl(1-methylcyclopropyl)carbamate

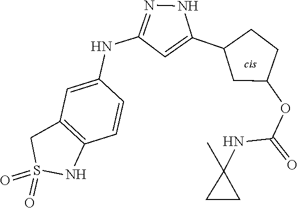 [SMILES: CC1(NC(=O)OC2CCC(c3cc(Nc4ccc5c(c4)CS(=O)(=O)N5)n[nH]3)C2)CC1]

The titled compound was synthesized in the procedures similar to Example 195. ¹H NMR (500 MHz, DMSO-d₆)) δ 11.61 (s, 1H), 9.73 (s, 1H), 8.17 (s, 1H), 7.35 (d, J=13.9 Hz, 2H), 7.15 (d, J=7.3 Hz, 1H), 6.70 (d, J=8.5 Hz, 1H), 5.57 (s, 1H), 4.97 (s, 1H), 4.43 (s, 2H), 3.05-2.95 (m, 1H), 2.55-2.45 (m, 1H), 2.05-1.98 (m, 1H), 1.94-1.87 (m, 1H), 1.71-1.65 (m, 2H), 1.58-1.48 (m, 1H), 1.23 (s, 3H), 0.59 (m, 2H), 0.47 (t, J=5.3 Hz, 2H). LC-MS (ESI): m/z [M+H]⁺=432.2.

### Example 233: cis-3-(3-((1-methyl-2,2-dioxido-1,3-dihydrobenzo[c]isothiazol-5-yl)amino)-1H-pyrazol-5-yl)cyclopentyl(1-methylcyclobutyl)carbamate

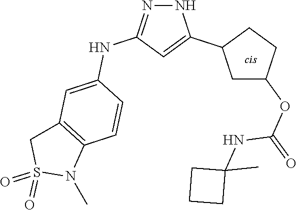 [SMILES: CN1c2ccc(Nc3cc(C4CCC(OC(=O)NC5(C)CCC5)C4)[nH]n3)cc2CS1(=O)=O]

The titled compound was synthesized in the procedures similar to Example 196. ¹H NMR (500 MHz, DMSO-d₆) δ 11.62 (s, 1H), 8.22 (s, 1H), 7.41 (s, 1H), 7.26 (d, J=7.1 Hz, 1H), 7.17 (s, 1H), 6.78 (d, J=8.6 Hz, 1H), 5.57 (s, 1H), 5.02-4.92 (m, 1H), 4.56 (s, 2H), 3.06-2.99 (m, 1H), 2.95 (s, 3H), 2.46-2.39 (m, 1H), 2.28-2.16 (m, 2H), 2.05-1.96 (m, 1H), 1.92-1.88 (m, 1H), 1.85-1.77 (m, 2H), 1.74-1.66 (m, 4H), 1.62-1.56 (m, 1H), 1.31 (s, 3H). LC-MS (ESI): m/z [M+H]⁺=460.2.

### Example 234: cis-3-(3-((1-methyl-2,2-dioxido-1,3-dihydrobenzo[c]isothiazol-5-yl)amino)-1H-pyrazol-5-yl)cyclopentyl(3-methyloxetan-3-yl)carbamate

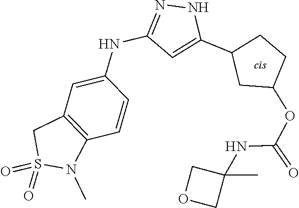 [SMILES: CN1c2ccc(Nc3cc(C4CCC(OC(=O)NC5(C)COC5)C4)[nH]n3)cc2CS1(=O)=O]

The titled compound was synthesized in the procedures similar to Example 196. ¹H NMR (500 MHz, DMSO-d₆) δ 11.71 (s, 1H), 8.22 (s, 1H), 7.63 (s, 1H), 7.40 (s, 1H), 7.25 (d, J=8.6 Hz, 1H), 6.78 (d, J=8.7 Hz, 1H), 5.58 (s, 1H), 5.01 (s, 1H), 4.56 (s, 4H), 4.24 (d, J=4.3 Hz, 2H), 3.11-2.98 (m, 1H), 2.95 (s, 3H), 2.48-2.44 (m, 1H), 2.07-1.97 (m, 1H), 1.97-1.86 (m, 1H), 1.79-1.66 (m, 2H), 1.63-1.53 (m, 1H), 1.47 (s, 3H). LC-MS (ESI): m/z [M+H]⁺=462.2.

### Example 235: cis-3-(3-((1-methyl-2,2-dioxido-1,3-dihydrobenzo[c]isothiazol-5-yl)amino)-1H-pyrazol-5-yl)cyclopentyl(1-(oxetan-3-yl)ethyl)carbamate

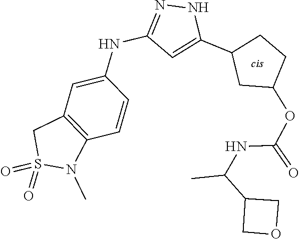 [SMILES: CC(NC(=O)OC1CCC(c2cc(Nc3ccc4c(c3)CS(=O)(=O)N4C)n[nH]2)C1)C1COC1]

The titled compound was synthesized in the procedures similar to Example 196. ¹H NMR (500 MHz, DMSO-d₆) δ 11.63 (s, 1H), 8.20 (d, J=6.0 Hz, 1H), 7.40 (s, 1H), 7.25 (d, J=8.1 Hz, 1H), 7.08 (d, J=8.4 Hz, 1H), 6.78 (d, J=8.7 Hz, 1H), 5.57 (d, J=2.6 Hz, 1H), 4.99 (s, 1H), 4.56 (s, 2H), 4.55-4.48 (m, 2H), 4.34 (t, J=5.5 Hz, 1H), 4.24 (t, J=6.1 Hz, 1H), 3.86-3.76 (m, 1H), 3.12-2.98 (m, 1H), 2.95 (s, 3H), 2.92-2.86 (m, 1H), 2.46-2.41 (m, 1H), 2.07-1.97 (m, 1H), 1.97-1.86 (m, 1H), 1.79-1.66 (m, 2H), 1.63-1.53 (m, 1H), 0.98-0.90 (m, 3H). LC-MS (ESI): m/z [M+H]⁺=476.2.

### Example 236: cis-3-(3-((1-methyl-2,2-dioxido-1,3-dihydrobenzo[c]isothiazol-5-yl)amino)-1H-pyrazol-5-yl)cyclopentyl oxetan-3-ylcarbamate

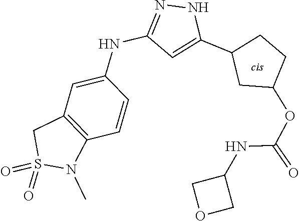 [SMILES: CN1c2ccc(Nc3cc(C4CCC(OC(=O)NC5COC5)C4)[nH]n3)cc2CS1(=O)=O]

The titled compound was synthesized in the procedures similar to Example 196. ¹H NMR (500 MHz, DMSO-d₆) δ 11.64 (s, 1H), 8.22 (s, 1H), 7.90 (d, J=4.9 Hz, 1H), 7.41 (s, 1H), 7.26 (d, J=7.2 Hz, 1H), 6.78 (d, J=8.6 Hz, 1H), 5.58 (s, 1H), 5.05-4.94 (m, 1H), 4.69-4.60 (m, 3H), 4.56 (s, 2H), 4.44-4.36 (m, 2H), 3.12-2.99 (m, 1H), 2.95 (s, 3H), 2.46-2.38 (m, 1H), 2.07-1.97 (m, 1H), 1.97-1.86 (m, 1H), 1.79-1.66 (m, 2H), 1.63-1.53 (m, 1H). LC-MS (ESI): m/z [M+H]⁺=448.2.

### Example 237: cis-3-(3-((1-methyl-2,2-dioxido-1,3-dihydrobenzo[c]isothiazol-5-yl)amino)-1H-pyrazol-5-yl)cyclopentyl(oxetan-3-ylmethyl)carbamate

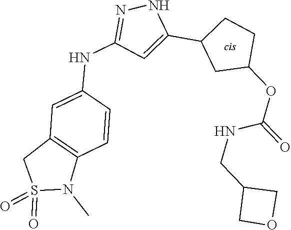 [SMILES: CN1c2ccc(Nc3cc(C4CCC(OC(=O)NCC5COC5)C4)[nH]n3)cc2CS1(=O)=O]

The titled compound was synthesized in the procedures similar to Example 196. ¹H NMR (500 MHz, DMSO-d₆) δ 11.63 (s, 1H), 8.21 (s, 1H), 7.41 (s, 1H), 7.25 (d, J=5.5 Hz, 2H), 6.78 (d, J=8.7 Hz, 1H), 5.57 (s, 1H), 5.00 (s, 1H), 4.60-4.55 (m, 4H), 4.25 (t, J=5.9 Hz, 2H), 3.25-3.22 (m, 1H), 3.05-2.98 (m, 2H), 2.95 (s, 3H), 2.48-2.44 (m, 1H), 2.04-1.97 (m, 1H), 1.95-1.84 (m, 1H), 1.79-1.66 (m, 2H), 1.63-1.53 (m, 1H). LC-MS (ESI): m/z [M+H]⁺=462.2.

### Example 238: cis-3-(3-((1-methyl-2,2-dioxido-1,3-dihydrobenzo[c]isothiazol-5-yl)amino)-1H-pyrazol-5-yl)cyclopentyl(2-hydroxy-2-methylpropyl)carbamate

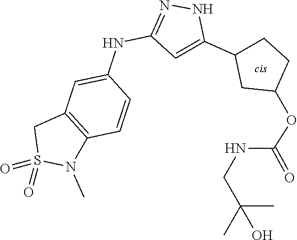 [SMILES: CN1c2ccc(Nc3cc(C4CCC(OC(=O)NCC(C)(C)O)C4)[nH]n3)cc2CS1(=O)=O]

The titled compound was synthesized in the procedures similar to Example 196. ¹H NMR (500 MHz, DMSO-d₆) δ 11.63 (s, 1H), 8.21 (s, 1H), 7.40 (s, 1H), 7.25 (d, J=7.1 Hz, 1H), 6.85 (d, J=6.0 Hz, 1H), 6.78 (d, J=8.6 Hz, 1H), 5.58 (s, 1H), 5.00 (s, 1H), 4.56 (s, 2H), 4.33 (s, 1H), 3.11-2.99 (m, 1H), 2.95 (s, 3H), 2.92 (d, J=6.2 Hz, 2H), 2.48-2.44 (m, 1H), 2.07-1.97 (m, 1H), 1.97-1.86 (m, 1H), 1.79-1.66 (m, 2H), 1.63-1.53 (m, 1H), 1.02 (s, 6H). LC-MS (ESI): m/z [M+H]⁺=464.2.

### Example 239: cis-3-(3-((1-methyl-2,2-dioxido-1,3-dihydrobenzo[c]isothiazol-5-yl)amino)-1H-pyrazol-5-yl)cyclopentyl(3-methyltetrahydrofuran-3-yl)carbamate

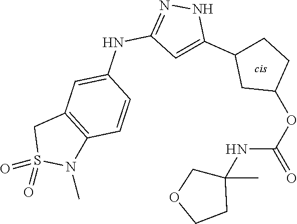 [SMILES: CN1c2ccc(Nc3cc(C4CCC(OC(=O)NC5(C)CCOC5)C4)[nH]n3)cc2CS1(=O)=O]

The titled compound was synthesized in the procedures similar to Example 196. ¹H NMR (500 MHz, DMSO-d₆) δ 11.64 (s, 1H), 8.22 (s, 1H), 7.40 (s, 1H), 7.24 (s, 2H), 6.78 (d, J=8.7 Hz, 1H), 5.57 (s, 1H), 5.02-4.92 (m, 1H), 4.56 (s, 2H), 3.81 (s, 1H), 3.78-3.68 (m, 2H), 3.43 (d, J=8.6 Hz, 1H), 3.10-2.99 (m, 1H), 2.95 (s, 3H), 2.46-2.38 (m, 1H), 2.20-2.10 (s, 1H), 2.06-1.98 (m, 1H), 1.94-1.86 (m, 1H), 1.78-1.68 (m, 3H), 1.64-1.54 (m, 1H), 1.32 (s, 3H). LC-MS (ESI): m/z [M+H]⁺=476.2.

### Example 240: cis-3-(3-((4-methyl-2,2-dioxido-1,3-dihydrobenzo[c]isothiazol-5-yl)amino)-1H-pyrazol-5-yl)cyclopentyl isopropylcarbamate

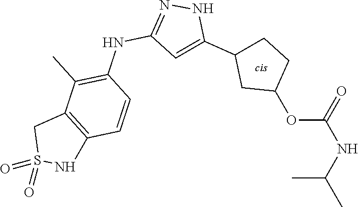 [SMILES: Cc1c(Nc2cc(C3CCC(OC(=O)NC(C)C)C3)[nH]n2)ccc2c1CS(=O)(=O)N2]

The titled compound was synthesized in the procedures similar to Example 203. ¹H NMR (500 MHz, DMSO-d₆) δ 11.60 (s, 1H), 9.93 (s, 1H), 7.45 (s, 1H), 7.09 (s, 1H), 6.93 (d, J=6.9 Hz, 1H), 6.57 (d, J=8.5 Hz, 1H), 5.58 (s, 1H), 4.98 (s, 1H), 4.45 (s, 2H), 3.66-3.51 (m, 1H), 3.16-2.93 (m, 1H), 2.45-2.35 (m, 1H), 2.09 (s, 3H), 2.02-1.95 (m, 1H), 1.93-1.84 (m, 1H), 1.69 (s, 3H), 1.57 (s, 1H), 1.02 (d, J=6.2 Hz, 6H). LC-MS (ESI): m/z [M+H]⁺=434.2

### Example 241:3-(3-((6-methyl-2,2-dioxido-1,3-dihydrobenzo[c]isothiazol-5-yl)amino)-1H-pyrazol-5-yl)cyclopentyl isopropylcarbamate

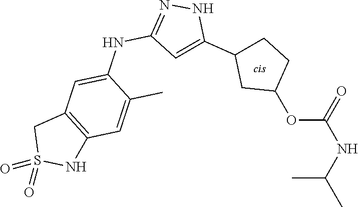 [SMILES: Cc1cc2c(cc1Nc1cc(C3CCC(OC(=O)NC(C)C)C3)[nH]n1)CS(=O)(=O)N2]

The titled compound was synthesized in the procedures similar to Example 203. ¹H NMR (500 MHz, DMSO-d₆) δ 11.68 (s, 1H), 9.77 (s, 1H), 7.62 (s, 1H), 7.06 (s, 1H), 6.93 (d, J=7.4 Hz, 1H), 6.62 (s, 1H), 5.66 (s, 1H), 4.99 (s, 1H), 4.39 (s, 2H), 3.66-3.53 (m, 1H), 3.11-2.93 (m, 1H), 2.45 (dd, J=13.9, 7.1 Hz, 1H), 2.19 (s, 3H), 2.00 (d, J=8.1 Hz, 1H), 1.93-1.82 (m, 1H), 1.70 (t, J=7.7 Hz, 2H), 1.58 (s, 1H), 1.13-0.88 (m, 6H). LC-MS (ESI): m/z [M+H]⁺=434.2.

### Example 242: cis-3-(3-((7-methyl-2,2-dioxido-1,3-dihydrobenzo[c]isothiazol-5-yl)amino)-1H-pyrazol-5-yl)cyclopentyl isopropylcarbamate

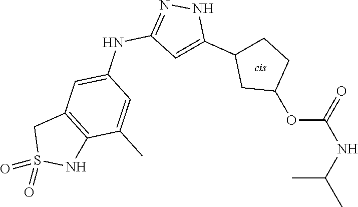 [SMILES: Cc1cc(Nc2cc(C3CCC(OC(=O)NC(C)C)C3)[nH]n2)cc2c1NS(=O)(=O)C2]

The titled compound was synthesized in the procedures similar to Example 203. ¹H NMR (500 MHz, DMSO-d₆) δ 11.67 (s, 1H), 9.42 (s, 1H), 8.13 (d, J=5.6 Hz, 1H), 7.19 (s, 1H), 7.02 (s, 1H), 6.95 (d, J=7.2 Hz, 1H), 5.56 (s, 1H), 4.99 (s, 1H), 4.43 (s, 2H), 3.57 (d, J=6.3 Hz, 1H), 3.12-2.89 (m, 1H), 2.47-2.40 (m, 1H), 2.10 (s, 3H), 2.00 (d, J=8.0 Hz, 1H), 1.94-1.84 (m, 1H), 1.71 (d, J=8.0 Hz, 2H), 1.58 (s, 1H), 1.03 (d, J=6.4 Hz, 6H). LC-MS (ESI): m/z [M+H]⁺=434.2.

### Example 243: cis-3-(3-((1,4-dimethyl-2,2-dioxido-1,3-dihydrobenzo[c]isothiazol-5-yl)amino)-1H-pyrazol-5-yl)cyclopentyl isopropylcarbamate

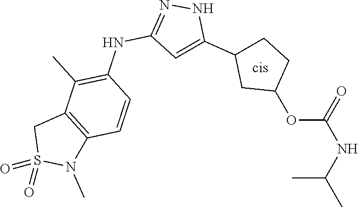 [SMILES: Cc1c(Nc2cc(C3CCC(OC(=O)NC(C)C)C3)[nH]n2)ccc2c1CS(=O)(=O)N2C]

The titled compound was synthesized in the procedures similar to Example 203. ¹H NMR (500 MHz, DMSO-d₆) δ 11.65 (s, 1H), 7.55 (s, 1H), 7.14 (s, 1H), 6.93 (d, J=7.2 Hz, 1H), 6.65 (d, J=8.6 Hz, 1H), 5.60 (s, 1H), 4.98 (s, 1H), 4.58 (s, 2H), 3.57 (dd, J=13.1, 6.5 Hz, 1H), 3.06-2.98 (m, 1H), 2.95 (s, 3H), 2.43 (dd, J=13.8, 7.2 Hz, 1H), 2.11 (s, 3H), 2.04-1.95 (m, 1H), 1.93-1.82 (m, 1H), 1.69 (s, 2H), 1.58 (s, 1H), 1.02 (d, J=6.1 Hz, 6H). LC-MS (ESI): m/z [M+H]⁺=448.2.

### Example 244: cis-3-(3-((1,6-dimethyl-2,2-dioxido-1,3-dihydrobenzo[c]isothiazol-5-yl)amino)-1H-pyrazol-5-yl)cyclopentyl isopropylcarbamate

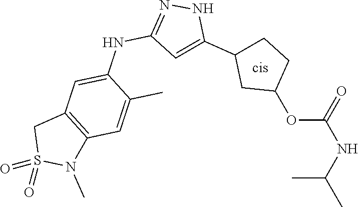 [SMILES: Cc1cc2c(cc1Nc1cc(C3CCC(OC(=O)NC(C)C)C3)[nH]n1)CS(=O)(=O)N2C]

The titled compound was synthesized in the procedures similar to Example 203. ¹H NMR (500 MHz, DMSO-d₆) δ 11.70 (s, 1H), 8.34 (s, 1H), 7.25 (s, 1H), 7.08 (s, 1H), 6.95 (d, J=7.3 Hz, 1H), 5.60 (s, 1H), 4.99 (s, 1H), 4.50 (s, 2H), 3.58 (dd, J=13.1, 6.4 Hz, 1H), 3.09-3.00 (m, 1H), 2.96 (s, 3H), 2.44 (dd, J=13.8, 7.1 Hz, 1H), 2.24 (s, 3H), 2.00 (dd, J=15.7, 7.6 Hz, 1H), 1.94-1.85 (m, 1H), 1.70 (t, J=12.2 Hz, 2H), 1.58 (s, 1H), 1.03 (d, J=6.3 Hz, 6H). LC-MS (ESI): m/z [M+H]⁺=448.2.

### Example 245: cis-3-(3-((1,7-dimethyl-2,2-dioxido-1,3-dihydrobenzo[c]isothiazol-5-yl)amino)-1H-pyrazol-5-yl)cyclopentyl isopropylcarbamate

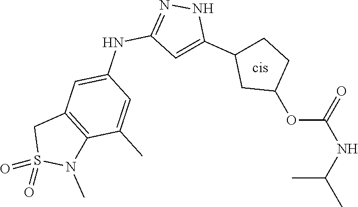 [SMILES: Cc1cc(Nc2cc(C3CCC(OC(=O)NC(C)C)C3)[nH]n2)cc2c1N(C)S(=O)(=O)C2]

The titled compound was synthesized in the procedures similar to Example 203. ¹H NMR (500 MHz, DMSO-d₆) δ 11.65 (s, 1H), 7.68 (s, 1H), 7.10 (s, 1H), 6.94 (d, J=7.4 Hz, 1H), 6.72 (s, 1H), 5.66 (s, 1H), 4.99 (s, 1H), 4.51 (s, 2H), 3.57 (dd, J=13.2, 6.4 Hz, 1H), 3.02 (s, 1H), 2.95 (s, 3H), 2.47-2.39 (m, 1H), 2.23 (s, 3H), 1.99 (s, 1H), 1.93-1.85 (m, 1H), 1.71 (d, J=7.8 Hz, 2H), 1.58 (s, 1H), 1.08-0.97 (m, 6H). LC-MS (ESI): m/z [M+H]⁺=448.2.

### Example 246: cis-3-(3-((2,2-dioxido-3,4-dihydro-1H-benzo[c][1,2]thiazin-6-yl)amino)-1H-pyrazol-5-yl)cyclopentyl isopropylcarbamate

### Step 1: 1-(4-methoxybenzyl)-1H-benzo[c][1,2]thiazine 2,2-dioxide

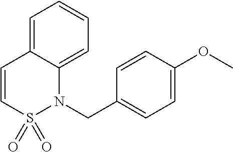 [SMILES: COc1ccc(CN2c3ccccc3C=CS2(=O)=O)cc1]

To a solution of N-(2-formylphenyl) methanesulfonamide (10 g, 50 mmol) in 100 mL MeCN was added 1-(chloromethyl)-4-methoxybenzene (16 g, 100 mmol) and Cs₂CO₃ (33 g, 100 mmol). The resulting solution was stirred at 50° C. under nitrogen for 48 h. The mixture was filtered, and the filtrate concentrated under vacuum. The residue was purified with silica gel column chromatography, eluting with EA/PE (20%) to afford the product (16.2 g, 54%). LC-MS (ESI): m/z [M+H]⁺=302.3.

### Step 2:1-(4-methoxybenzyl)-3,4-dihydro-1H-benzo[c][1,2]thiazine 2,2-dioxide

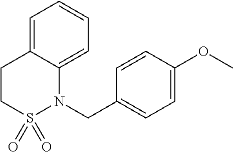 [SMILES: COc1ccc(CN2c3ccccc3CCS2(=O)=O)cc1]

To a solution of 1-(4-methoxybenzyl)-1H-benzo[c][1,2]thiazine 2,2-dioxide (3.4 g, 11.2 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (3.2 g, 94% yield). LC-MS (ESI): m/z [M+H]⁺=304.3.

### Step 3: 3,4-dihydro-1H-benzo[c][1,2]thiazine 2,2-dioxide

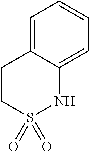 [SMILES: O=S1(=O)CCc2ccccc2N1]

To a solution of 1-(4-methoxybenzyl)-3,4-dihydro-1H-benzo[c][1,2]thiazine 2,2-dioxide (3.2 g, 10.5 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (3.2 g, 94% yield). LC-MS (ESI): m/z [M+H]⁺=184.3.

### Step 4: 6-bromo-3,4-dihydro-1H-benzo[c][1 2]thiazine 2,2-dioxide

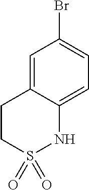 [SMILES: O=S1(=O)CCc2cc(Br)ccc2N1]

To a solution of 3,4-dihydro-1H-benzo[c][1,2]thiazine 2,2-dioxide (1.7 g, 9.2 mmol) in DMF (100 mL) was added NBS (1.16 g, 6.5 mmol). The mixture was stirred for 12 h at room temperature. Water (200 mL) was added and extracted with EA (3×100 mL). The organic layers were collected and concentrated under vacuum. The residue was purified by silica gel column chromatography, eluting with EA/PE (50%) to afford the product (1.1 g, 46%). LC-MS (ESI): m/z [M+H]⁺=262.5.

### Step 5: 6-bromo-1-(4-methoxybenzyl)-3,4-dihydro-1H-benzo [c][1 2]thiazine 2,2-dioxide

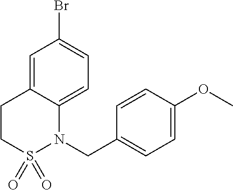 [SMILES: COc1ccc(CN2c3ccc(Br)cc3CCS2(=O)=O)cc1]

To a solution of 6-bromo-3,4-dihydro-1H-benzo[c][1,2]thiazine 2,2-dioxide (600 mg, 2.3 mmol) in DMF (20 mL) was added NaH (138 mg, 5.75 mmol) at 0° C. The mixture was stirred for 30 min at room temperature, and 1-(bromomethyl)-4-methoxybenzene (555 mg, 2.76 mmol) was added dropwise. Water (50 mL) was added and extracted with EA (3×30 mL). The organic layers were collected and concentrated under vacuum. The residue was purified by silica gel column chromatography, eluting with EA/PE (20%) to afford the product (666 mg, 76%). LC-MS (ESI): m/z [M+H]⁺=382.5.

### Step 6: cis-3-(3-((1-(4-methoxybenzyl)-2,2-dioxido-3,4-dihydro-1H-benzo[c][1,2]thiazin-6-yl)amino)-1H-pyrazol-5-yl)cyclopentyl isopropylcarbamate

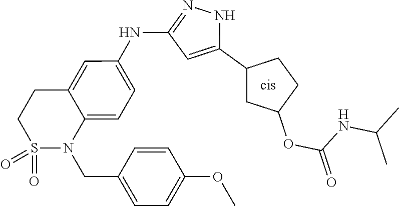 [SMILES: COc1ccc(CN2c3ccc(Nc4cc(C5CCC(OC(=O)NC(C)C)C5)[nH]n4)cc3CCS2(=O)=O)cc1]

The titled compound was synthesized in the procedures similar to Example 34, step 5. LC-MS (ESI): m/z [M+H]⁺=554.4.

### Step 7: cis-3-(3-((2,2-dioxido-3,4-dihydro-1H-benzo[c][1.2]thiazin-6-yl)amino)-1H-pyrazol-5-yl)cyclopentyl isopropylcarbamate

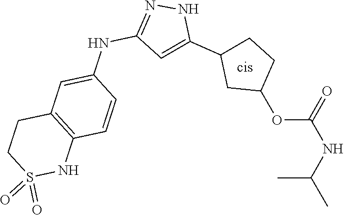 [SMILES: CC(C)NC(=O)OC1CCC(c2cc(Nc3ccc4c(c3)CCS(=O)(=O)N4)n[nH]2)C1]

To a flask charged with cis-3-(3-((1-(4-methoxybenzyl)-2,2-dioxido-3,4-dihydro-1H-benzo[c][1,2]thiazin-6-yl)amino)-1H-pyrazol-5-yl)cyclopentyl isopropylcarbamate (123 mg, 0.22 mmol) was added TFA (1 mL) and DCM (3 mL). The result solution was stirred at room temperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product in racemic form, which was further separated with chiral Prep-HPLC to give:

Enantiomer 1 (Example 246a, 100% ee); Retention time: 4.352 min. ¹H NMR (500 MHz, DMSO-d₆) δ 11.62 (s, 1H), 9.41 (s, 1H), 8.11 (s, 1H), 7.21 (s, 1H), 7.09 (d, J=7.7 Hz, 1H), 6.94 (d, J=7.4 Hz, 1H), 6.61 (d, J=8.7 Hz, 1H), 5.56 (s, 1H), 4.98 (s, 1H), 3.63-3.47 (m, 1H), 3.24 (dd, J=13.0, 5.2 Hz, 4H), 3.05-2.94 (m, 1H), 2.47-2.40 (m, 1H), 1.99 (dd, J=15.2, 7.3 Hz, 1H), 1.94-1.83 (m, 1H), 1.78-1.64 (m, 2H), 1.64-1.53 (m, 1H), 1.03 (d, J=6.3 Hz, 6H). LC-MS (ESI): m/z [M+H]⁺=434.4.

Enantiomer 2 (Example 246b, 100% ee); Retention time: 5.961 min. ¹H NMR (500 MHz, DMSO-d₆) δ 11.62 (s, 1H), 9.41 (s, 1H), 8.11 (s, 1H), 7.21 (s, 1H), 7.09 (d, J=7.7 Hz, 1H), 6.94 (d, J=7.4 Hz, 1H), 6.61 (d, J=8.7 Hz, 1H), 5.56 (s, 1H), 4.98 (s, 1H), 3.63-3.47 (m, 1H), 3.24 (dd, J=13.0, 5.2 Hz, 4H), 3.05-2.94 (m, 1H), 2.47-2.40 (m, 1H), 1.99 (dd, J=15.2, 7.3 Hz, 1H), 1.94-1.83 (m, 1H), 1.78-1.64 (m, 2H), 1.64-1.53 (m, 1H), 1.03 (d, J=6.3 Hz, 6H). LC-MS (ESI): m/z [M+H]⁺=434.4.

Chiral analytical method: Column: CHIRALPAK IE 4.6×250 mm 5 μm; Mobile phase: A for MtBE (0.1% 2M NH₃ MeOH) and B for MeOH: DCM=50:50 (v/v); Gradient: Mobile Phase A: Mobile Phase B=50: 50 (v/v); HPLC Equipment: HPLC-Agilent; Column temperature: 35° C.

Chiral Prep-HPLC Condition: CHIRALPAK IE 21.2 mm×250 mm, 5 μm; Mobile phase: A for MtBE and B for DCM:MeOH=50:50 (v/v) (0.2% 2 M NH₃. MeOH); Gradient: Mobile Phase A: Mobile Phase B=50: 50 (v/v); Flow Rate: 18 mL/min, Wavelength: UV 280 nm and 300 nm, Prep-HPLC Equipment: Prep-HPLC-Gilson; Column temperature: 25° C.

### Example 247: cis-3-(3-((2,2-dioxido-3,4-dihydro-1H-benzo[c][1,2]thiazin-6-yl)amino)-1H-pyrazol-5-yl)cyclopentyl propylcarbamate

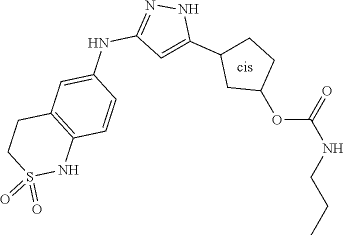 [SMILES: CCCNC(=O)OC1CCC(c2cc(Nc3ccc4c(c3)CCS(=O)(=O)N4)n[nH]2)C1]

The titled compound was synthesized in the procedures similar to Example 246. ¹H NMR (500 MHz, DMSO-d₆) δ 11.62 (s, 1H), 9.41 (s, 1H), 8.12 (d, J=10.3 Hz, 1H), 7.21 (s, 1H), 7.09 (d, J=7.6 Hz, 1H), 7.04 (t, J=5.6 Hz, 1H), 6.61 (d, J=8.7 Hz, 1H), 5.57 (s, 1H), 4.99 (s, 1H), 3.28-3.19 (m, 5H), 3.07-2.96 (m, 1H), 2.91 (dd, J=13.1, 6.6 Hz, 2H), 2.43 (dd, J=13.7, 7.1 Hz, 2H), 2.05-1.95 (m, 1H), 1.88 (dd, J=17.4, 10.2 Hz, 1H), 1.69 (dd, J=16.4, 8.7 Hz, 2H), 1.62-1.53 (m, 1H), 1.45-1.32 (m, 2H), 0.82 (t, J=7.4 Hz, 3H). LC-MS (ESI): m/z [M+H]⁺=434.4.

### Example 248: cis-3-(3-((2,2-dioxido-3,4-dihydro-1H-benzo[c][1,2]thiazin-6-yl)amino)-1H-pyrazol-5-yl)cyclopentyl((S)-sec-butyl)carbamate

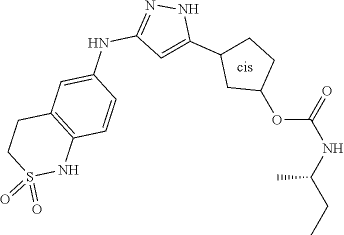 [SMILES: CC[C@H](C)NC(=O)OC1CCC(c2cc(Nc3ccc4c(c3)CCS(=O)(=O)N4)n[nH]2)C1]

The titled compound was synthesized in the procedures similar to Example 246. ¹H NMR (500 MHz, DMSO-d₆) δ 11.62 (s, 1H), 9.41 (s, 1H), 8.11 (s, 1H), 7.22 (s, 1H), 7.09 (d, J=7.8 Hz, 1H), 6.88 (d, J=8.4 Hz, 1H), 6.61 (d, J=8.7 Hz, 1H), 5.57 (s, 1H), 4.98 (s, 1H), 3.37 (dd, J=14.3, 6.8 Hz, 1H), 3.24 (dd, J=13.0, 5.2 Hz, 4H), 3.07-2.93 (m, 1H), 2.48-2.40 (m, 2H), 2.00 (dd, J=15.9, 7.7 Hz, 1H), 1.94-1.84 (m, 1H), 1.70 (dt, J=15.7, 7.7 Hz, 2H), 1.63-1.53 (m, 1H), 1.44-1.26 (m, 2H), 1.00 (d, J=6.4 Hz, 3H), 0.81 (dt, J=11.9, 5.9 Hz, 3H). LC-MS (ESI): m/z [M+H]⁺=448.2.

### Example 249: cis-3-(3-((2,2-dioxido-3,4-dihydro-1H-benzo[c][1,2]thiazin-6-yl)amino)-1H-pyrazol-5-yl)cyclopentyl(1-methylcyclopropyl)carbamate

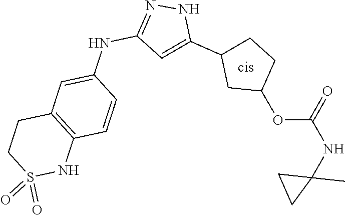 [SMILES: CC1(NC(=O)OC2CCC(c3cc(Nc4ccc5c(c4)CCS(=O)(=O)N5)n[nH]3)C2)CC1]

The titled compound was synthesized in the procedures similar to Example 246 in a racemic form, which was separated by Chiral Prep-HPLC to give:

Enantiomer 1 (Example 249a, 100% ee); Retention time: 5.453 min. ¹H NMR (500 MHz, DMSO-d₆) δ 11.61 (s, 1H), 9.40 (s, 1H), 8.11 (s, 1H), 7.34 (s, 1H), 7.22 (s, 1H), 7.09 (dd, J=6.2, 2.7 Hz, 1H), 6.61 (d, J=8.7 Hz, 1H), 5.56 (s, 1H), 4.98 (s, 1H), 3.24 (dd, J=13.3, 5.2 Hz, 4H), 3.06-2.96 (m, 1H), 2.46-2.39 (m, 1H), 1.98 (dd, J=14.2, 7.3 Hz, 1H), 1.89 (d, J=9.5 Hz, 1H), 1.67 (dd, J=11.2, 5.2 Hz, 2H), 1.55 (dd, J=17.0, 3.6 Hz, 1H), 1.23 (s, 3H), 0.59 (s, 2H), 0.47 (q, J=4.6 Hz, 2H). LC-MS (ESI): m/z [M+H]⁺=446.2.

Enantiomer 2 (Example 249b, 100% ee); Retention time: 8.619 min. ¹H NMR (500 MHz, DMSO-d₆) δ 11.61 (s, 1H), 9.40 (s, 1H), 8.11 (s, 1H), 7.34 (s, 1H), 7.22 (s, 1H), 7.09 (dd, J=6.2, 2.7 Hz, 1H), 6.61 (d, J=8.7 Hz, 1H), 5.56 (s, 1H), 4.98 (s, 1H), 3.24 (dd, J=13.3, 5.2 Hz, 4H), 3.06-2.96 (m, 1H), 2.46-2.39 (m, 1H), 1.98 (dd, J=14.2, 7.3 Hz, 1H), 1.89 (d, J=9.5 Hz, 1H), 1.67 (dd, J=11.2, 5.2 Hz, 2H), 1.55 (dd, J=17.0, 3.6 Hz, 1H), 1.23 (s, 3H), 0.59 (s, 2H), 0.47 (q, J=4.6 Hz, 2H). LC-MS (ESI): m/z [M+H]⁺=446.2.

Chiral analytical method: Column: CHIRALPAK IE 4.6×250 mm 5 μm; Mobile phase: A for Hexane and B for EtOH (0.1% 2M NH₃ MeOH); Gradient: Mobile Phase A: Mobile Phase B=20: 80 (v/v); HPLC Equipment: HPLC-Agilent; Column temperature: 35° C.

Chiral Prep-HPLC Condition: CHIRALPAK IE 21.2 mm×250 mm, 5 μm; Mobile phase: A for Hexane and B for EtOH (0.2% 2M NH₃ MeOH); Gradient: Mobile Phase A: Mobile Phase B=50: 50 (v/v); Flow Rate: 18 mL/min, Wavelength: UV 280 nm and 300 nm, Prep-HPLC Equipment: Prep-HPLC-Gilson; Column temperature: 25° C.

### Example 250: cis-3-(3-((1-methyl-2,2-dioxido-3,4-dihydro-1H-benzo[c][1,2]thiazin-6-yl)amino)-1H-pyrazol-5-yl)cyclopentyl isopropylcarbamate

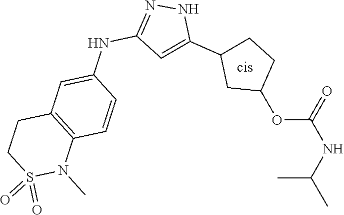 [SMILES: CC(C)NC(=O)OC1CCC(c2cc(Nc3ccc4c(c3)CCS(=O)(=O)N4C)n[nH]2)C1]

The titled compound was synthesized in the procedures similar to Example 259. ¹H NMR (500 MHz, DMSO-d₆) δ 11.71 (s, 1H), 8.23 (d, J=17.8 Hz, 1H), 7.22 (s, 1H), 7.17 (d, J=8.8 Hz, 1H), 6.95 (d, J=8.8 Hz, 2H), 5.59 (s, 1H), 4.99 (s, 1H), 3.57 (dd, J=13.1, 6.7 Hz, 1H), 3.41 (t, J=6.9 Hz, 2H), 3.26-3.19 (m, 2H), 3.11 (s, 3H), 3.02 (dt, J=17.2, 8.4 Hz, 1H), 2.48-2.41 (m, 1H), 2.06-1.96 (m, 1H), 1.95-1.83 (m, 1H), 1.79-1.65 (m, 2H), 1.63-1.52 (m, 1H), 1.03 (d, J=6.4 Hz, 6H). LC-MS (ESI): m/z [M+H]⁺=448.3.

### Example 251: cis-3-(3-((4-fluoro-2,2-dioxido-1,3-dihydrobenzo[c]isothiazol-5-yl)amino)-1H-pyrazol-5-yl)cyclopentyl isopropylcarbamate

### Step 1: S-(2-chloro-6-fluorobenzyl)ethanethioate

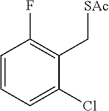

To the solution of 1-chloro-2-(chloromethyl)-3-fluorobenzene (100.0 g, 561.8 mmol, 1 eq) in DMF (1.0 L), was added Potassium thioacetate (128.3 g, 1123.6 mmol, 2.0 eq) at 0° C. Then the reaction was warmed to room temperature and stirred for 1 h. Diluted with water (1 L), extracted with EtOAc (600 mL×3), washed with brine (600 mL×5), dried over Na₂SO₄, concentrated to give product (120.0 g, yield 97.8%). ¹H NMR (300 MHz, DMSO-d₆) δ 7.42-7.33 (m, 2H), 7.27-7.21 (m, 1H), 4.26 (s, 2H), 2.36 (s, 3H).

### Step 2: (2-chloro-6-fluorophenyl)methanesulfonyl chloride

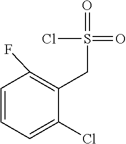 [SMILES: O=S(=O)(Cl)Cc1c(F)cccc1Cl]

To the solution of NCS (244.3 g, 1829.2 mmol, 4.0 eq) in 2N HCl (150 mL) and MeCN (500 mL), a solution of S-(2-chloro-6-fluorobenzyl)ethanethioate (100.0 g, 457.2 mmol, 1.0 eq) in MeCN (500 mL) was added dropwise below 20° C. The mixture was stirred at room temperature for 2 h. Concentrated and diluted with water (200 mL), extracted with EtOAc (300 mL×3), washed with brine (300 mL×2), dried over Na₂SO₄ and concentrated to give product (130.0 g, crude).

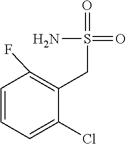 [SMILES: NS(=O)(=O)Cc1c(F)cccc1Cl]

### Step 3: (2-chloro-6-fluorophenyl) methanesulfonamide

Dissolved the (2-chloro-6-fluorophenyl) methanesulfonyl chloride (130.0 g, 534.8 mmol, 1.0 eq) in THF (1.3 L), the mixture was added dropwise to Ammonium hydroxide (28%) (240 mL) at 0° C., then the mixture was stirred at room temperature for 2 h. Then diluted with water (600 mL), extracted with EtOAc (500 mL×3), dried over Na₂SO₄, concentrated to get residue. The residue was triturated with MeCN (200 mL), filtered and the cake was dried under vacuum to give product (72.0 g, yield 68.9% over 2 steps). ¹H NMR (300 MHz, DMSO-d₆) δ 7.48-7.37 (m, 2H), 7.30-7.25 (m, 1H), 7.19 (brs, 2H), 4.48 (s, 2H). LC-MS (ESI): m/z [M+NH₄]⁺=241.0.

### Step 4:4-fluoro-1,3-dihydrobenzo[c]isothiazole 2,2-dioxide

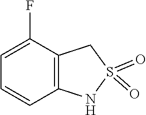 [SMILES: O=S1(=O)Cc2c(F)cccc2N1]

Under N₂ atmosphere, to the solution of (2-chloro-6-fluorophenyl) methanesulfonamide (80.0 g, 357.7 mmol, 1.0 eq) in DMF (800 mL), was added CuI (20.4 g, 107.3 mmol, 0.3 eq), K₃PO₄ (379.6 g, 1788.5 mmol, 4.0 eq) and Sarcosine (159.3 g, 1788 mmol, 5.0 eq). The mixture was stirred at 100° C. for 8h. Cooled and diluted with water (800 mL), adjusted pH to 2 with HCl (2N), extracted with EtOAc (800 mL×5), washed with brine (800 mL×5), dried over Na₂SO₄, concentrated to get a residue. The residue was triturated with DCM (300 mL), filtered and the cake was dried under vacuum to give product (52.0 g, yield 77.7%). ¹H NMR (400 MHz, CDCl₃) δ 10.88 (brs, 1H), 7.34-7.27 (m, 1H), 6.83 (t, J=8.7 Hz, 1H), 6.67 (d, J=8.1 Hz, 1H), 4.63 (s, 2H). LC-MS (ESI): m/z [M−H]⁻=186.0.

### Step 5:5-bromo-4-fluoro-1,3-dihydrobenzo[c]isothiazole 2,2-dioxide

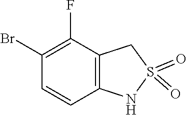 [SMILES: O=S1(=O)Cc2c(ccc(Br)c2F)N1]

Under N2 atmosphere, to a solution of 4-fluoro-1,3-dihydrobenzo[c]isothiazole 2,2-dioxide (40.0 g, 213.7 mmol, 1.0 eq) in AcOH (200 mL), was added dropwise a solution of Br₂ (37.1 g, 235.0 mmol, 1.1 eq) in AcOH (200 mL). Then the mixture was stirred at 15° C. for 3 h. Quenched with 10% Na₂S₂O₃ (400 mL), and ice water (400 mL) was added, the mixture was filtered and the cake was washed with ice water (200 mL×2), the cake was triturated with MeCN (100 mL), and DCM (100 mL), filtered and the cake was dried under vacuum to give 5-bromo-4-fluoro-1,3-dihydrobenzo[c]isothiazole 2,2-dioxide (29.0 g, yield 51.0%). ¹H NMR (400 MHz, DMSO-d₆): δ 11.08 (brs, 1H), 7.58 (t, J=8.0 Hz, 1H), 6.65 (d, J=8.8 Hz, 1H), 4.74 (s, 2H). LC-MS (ESI): m/z [M+H]⁺=265.9.

### Step 6:5-bromo-4-fluoro-1-(4-methoxybenzyl)-1,3-dihydrobenzo[c]isothiazole 2,2-dioxide

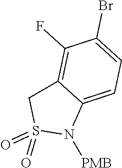

To a solution of 5-bromo-4-fluoro-1,3-dihydrobenzo[c]isothiazole 2,2-dioxide (500 mg, 1.89 mmol) in DMF (10 mL) was added K₂CO₃ (522 mg, 3.78 mmol) and 4-methoxybenzylchloride (444 mg, 2.8 mmol) at 0° C. The result mixture was stirred at room temperature for 2 h. The solution was poured into 20 mL water and extracted with EA. EA layers were combined, dried and concentrated under vacuum. The residue was applied to a silica gel column chromatography, eluting with PE/EtOAc (4:1) to afford the product (512 mg, 70%), LC-MS (ESI): m/z [M+Na]⁺=408.2.

### Step 7: cis-3-(3-((4-fluoro-1-(4-methoxybenzyl)-2,2-dioxido-1,3-dihydrobenzo[c]isothiazol-5-yl)amino)-1H-pyrazol-5-yl)cyclopentyl isopropylcarbamate

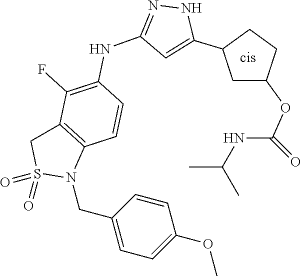 [SMILES: COc1ccc(CN2c3ccc(Nc4cc(C5CCC(OC(=O)NC(C)C)C5)[nH]n4)c(F)c3CS2(=O)=O)cc1]

A mixture of 5-bromo-4-fluoro-1-(4-methoxybenzyl)-1,3-dihydrobenzo[c]isothiazole 2,2-dioxide (177 mg, 0.46 mmol), cis-3-(3-amino-1H-pyrazol-5-yl)cyclopentyl isopropylcarbamate (116 mg, 0.46 mmol), Brettphos Pd G3 (41.4 mg, 0.046 mmol), K₂CO₃ (207 mg, 1.5 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product (70 mg, 27.5%). LC-MS (ESI): m/z [M+H]⁺=558.2.

### Step 8: cis-3-(3-((4-fluoro-2,2-dioxido-1,3-dihydrobenzo[c]isothiazol-5-yl)amino)-1H-pyrazol-5-yl)cyclopentyl isopropylcarbamate

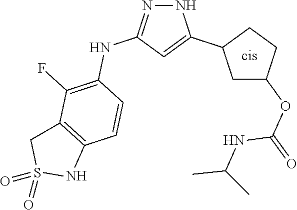 [SMILES: CC(C)NC(=O)OC1CCC(c2cc(Nc3ccc4c(c3F)CS(=O)(=O)N4)n[nH]2)C1]

To a stirred solution of cis-3-(3-((4-fluoro-1-(4-methoxybenzyl)-2,2-dioxido-1,3-dihydrobenzo[c]isothiazol-5-yl)amino)-1H-pyrazol-5-yl)cyclopentyl isopropylcarbamate (70 mg, 0.125 mmol) in DCM (5 mL) was added TFA (3 mL). The solution was stirred at room temperature for 12 h. The solution was concentrated under reduced pressure. The residue was purified by prep HPLC (Waters SunFire C18: RD-CO-058 column, eluting with a gradient of acetonitrile/water containing 0.1% FA, 20%-55%) to afford the product in racemic form, which was further separated by Chiral Prep-HPLC to give:

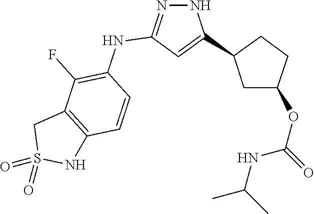 [SMILES: CC(C)NC(=O)O[C@@H]1CC[C@H](c2cc(Nc3ccc4c(c3F)CS(=O)(=O)N4)n[nH]2)C1]

Enantiomer 1 (Example 251a, (1R,3S)-3-(3-((4-fluoro-2,2-dioxido-1,3-dihydrobenzo[c]isothiazol-5-yl)amino)-1H-pyrazol-5-yl)cyclopentyl isopropylcarbamate, 100% ee); Retention time: 5.219 min. ¹H NMR (500 MHz, DMSO-d₆) δ 11.67 (s, 1H), 10.20 (s, 1H), 7.94 (s, 1H), 7.86 (s, 1H), 6.94 (d, J=7.3 Hz, 1H), 6.59 (d, J=8.6 Hz, 1H), 5.67 (s, 1H), 4.99 (s, 1H), 4.59 (s, 2H), 3.62-3.52 (m, 1H), 3.12-2.92 (m, 1H), 2.46-2.37 (m, 1H), 2.04-1.94 (m, 1H), 1.94-1.83 (m, 1H), 1.76-1.64 (m, 2H), 1.62-1.53 (m, 1H), 1.03 (d, J=6.4 Hz, 6H). LC-MS (ESI): m/z [M+H]⁺=438.2.

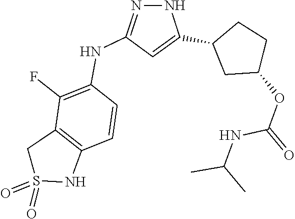 [SMILES: CC(C)NC(=O)O[C@H]1CC[C@@H](c2cc(Nc3ccc4c(c3F)CS(=O)(=O)N4)n[nH]2)C1]

Enantiomer 2 (Example 251b, (1S,3R)-3-(3-((4-fluoro-2,2-dioxido-1,3-dihydrobenzo[c]isothiazol-5-yl)amino)-1H-pyrazol-5-yl)cyclopentyl isopropylcarbamate, 100% ee); Retention time: 5.377 min. ¹H NMR (500 MHz, DMSO-d₆) δ 11.67 (s, 1H), 10.20 (s, 1H), 7.94 (s, 1H), 7.86 (s, 1H), 6.94 (d, J=7.3 Hz, 1H), 6.59 (d, J=8.6 Hz, 1H), 5.67 (s, 1H), 4.99 (s, 1H), 4.59 (s, 2H), 3.62-3.52 (m, 1H), 3.12-2.92 (m, 1H), 2.46-2.37 (m, 1H), 2.04-1.94 (m, 1H), 1.94-1.83 (m, 1H), 1.76-1.64 (m, 2H), 1.62-1.53 (m, 1H), 1.03 (d, J=6.4 Hz, 6H). LC-MS (ESI): m/z [M+H]⁺=438.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=60: 40 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 252: cis-3-(3-((4-fluoro-1-(2-methoxyethyl)-2,2-dioxido-1,3-dihydrobenzo[c]isothiazol-5-yl)cyclopentyl isopropylcarbamate

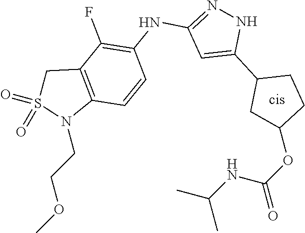 [SMILES: COCCN1c2ccc(Nc3cc(C4CCC(OC(=O)NC(C)C)C4)[nH]n3)c(F)c2CS1(=O)=O]

The titled compound was synthesized in the procedures similar to Example 35. ¹H NMR (500 MHz, DMSO-d₆) ¹H NMR (500 MHz, DMSO-d₆) δ 11.69 (s, 1H), 8.00 (t, J=8.2 Hz, 1H), 7.89 (s, 1H), 6.94 (d, J=7.5 Hz, 1H), 6.76 (d, J=8.8 Hz, 1H), 5.67 (s, 1H), 4.69 (s, 2H), 3.66-3.60 (m, 2H), 3.59-3.47 (m, 3H), 3.10 (s, 3H), 3.09-2.95 (m, 1H), 2.46-2.38 (m, 2H), 2.05-1.96 (m, 1H), 1.96-1.85 (m, 1H), 1.75-1.65 (m, 2H), 1.58-1.52 (m, 1H), 1.03 (d, J=6.2 Hz, 6H). LC-MS (ESI): m/z [M+H]⁺=496.2.

### Example 253: cis-3-(3-((4-fluoro-1,3-bis(2-hydroxyethyl)-2,2-dioxido-1,3-dihydrobenzo[c]isothiazol-5-yl)amino)-1H-pyrazol-5-yl)cyclopentyl isopropylcarbamate

### Step 1:5-bromo-1,3-bis(2-((tert-butyldimethylsilyl)oxy) ethyl)-4-fluoro-1,3-dihydrobenzo[c]isothiazole 2,2-dioxide

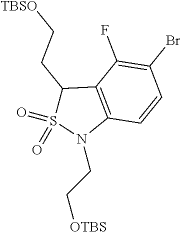 [SMILES: CC(C)(C)[Si](C)(C)OCCC1c2c(ccc(Br)c2F)N(CCO[Si](C)(C)C(C)(C)C)S1(=O)=O]

To a solution of 5-bromo-4-fluoro-1,3-dihydrobenzo[c]isothiazole 2,2-dioxide (500 mg, 1.89 mmol) in 20 mL DMF was added (2-bromoethoxy) (tert-butyl)dimethylsilane (1.2 g, 5 mmol) and Cs₂CO₃ (1.6 g, 5 mmol). The result mixture was stirred at room temperature for 5h. Water (20 mL) was added, and the resulting mixture was extracted with EA (3×50 mL). The organic layers were collected, dried and concentrated under vacuum. The residue was purified by silica gel column chromatography, eluting with EA/PE (10%) to afford the product (505 mg, 46%). LC-MS (ESI): m/z [M+H]⁺=582.3.

### Step 2: cis-3-(3-((1,3-bis(2-((tert-butyldimethylsilyl)oxy) ethyl)-4-fluoro-2,2-dioxido-1,3-dihydrobenzo[c]isothiazol-5-yl)amino)-1H-pyrazol-5-yl)cyclopentyl isopropylcarbamate

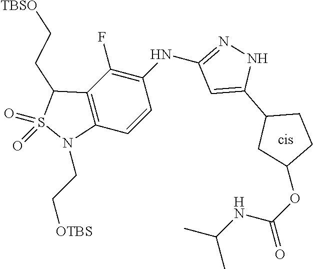 [SMILES: CC(C)NC(=O)OC1CCC(c2cc(Nc3ccc4c(c3F)C(CCO[Si](C)(C)C(C)(C)C)S(=O)(=O)N4CCO[Si](C)(C)C(C)(C)C)n[nH]2)C1]

The titled compound was synthesized in the procedures similar to Example 34. LC-MS (ESI): m/z [M+H]⁺=754.4.

### Step 3: cis-3-(3-((4-fluoro-1,3-bis(2-hydroxyethyl)-2,2-dioxido-1,3-dihydrobenzo[c]isothiazol-5-yl)amino-1H-pyrazol-5-yl)cyclopentyl isopropylcarbamate

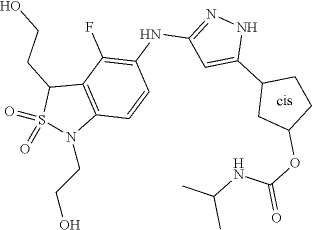 [SMILES: CC(C)NC(=O)OC1CCC(c2cc(Nc3ccc4c(c3F)C(CCO)S(=O)(=O)N4CCO)n[nH]2)C1]

To a round bottom flask charged with cis-3-(3-((1,3-bis(2-((tert-butyldimethylsilyl)oxy) ethyl)-4-fluoro-2,2-dioxido-1,3-dihydrobenzo[c]isothiazol-5-yl)amino)-1H-pyrazol-5-yl)cyclopentyl isopropylcarbamate (25 mg, 0.033 mmol) was added TFA (1 mL) and DCM (3 mL). The resulting mixture was stirred at room temperature for 1 h and concentrated under vacuum. The residue was purified by silica gel column chromatography, eluting with EA/PE (10% to 100%) to afford the product (12 mg, 69%). ¹H NMR (500 MHz, DMSO-d₆) δ 11.72 (s, 1H), 7.97 (s, 1H), 7.90 (s, 1H), 6.94 (d, J=6.9 Hz, 1H), 6.78 (d, J=8.8 Hz, 1H), 5.68 (s, 1H), 4.98 (s, 1H), 4.94 (t, J=5.4 Hz, 1H), 4.86 (t, J=5.2 Hz, 1H), 4.74-4.66 (m, 1H), 3.70-3.50 (m, 7H), 3.09-2.97 (m, 1H), 2.50-2.44 (m, 1H), 2.22-2.10 (m, 1H), 2.01-1.95 (m, 2H), 1.95-1.84 (m, 1H), 1.75-1.65 (m, 2H), 1.58-1.52 (m, 1H), 1.03 (d, J=6.2 Hz, 6H).

LC-MS (ESI): m/z [M+H]⁺=526.2.

### Example 254: cis-3-(3-((4-fluoro-1-(2-hydroxy-2-methylpropyl)-2,2-dioxido-1,3-dihydrobenzo[c]isothiazol-5-yl)amino)-1H-pyrazol-5-yl)cyclopentyl isopropylcarbamate

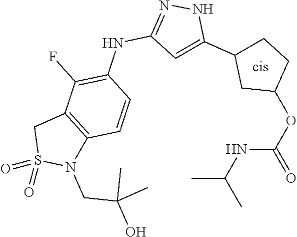 [SMILES: CC(C)NC(=O)OC1CCC(c2cc(Nc3ccc4c(c3F)CS(=O)(=O)N4CC(C)(C)O)n[nH]2)C1]

The titled compound was synthesized in the procedures similar to Example 63 in a racemic form, which was further separated by Chiral Prep-HPLC to give:

Enantiomer 1 (100% ee); Retention time: 3.962 min. ¹H NMR (500 MHz, DMSO-d₆) δ=11.69 (s, 1H), 7.97 (t, J=8.9, 1H), 7.88 (s, 1H), 6.92 (m, 2H), 5.67 (s, 1H), 4.99 (m, 1H), 4.70 (s, 2H), 4.66 (s, 1H), 3.57 (m, 1H), 3.32 (s, 2H), 3.02 (m, 1H), 2.44 (m, 1H), 2.00 (m, 1H), 1.90 (m, 1H), 1.70 (m, 2H), 1.57 (m, 1H), 1.20 (s, 6H), 1.03 (m, 6H). LC-MS (ESI): m/z [M+H]⁺=510.32.

Enantiomer 2 (100% ee); Retention time: 4.172 min. ¹H NMR (500 MHz, DMSO-d₆) δ=11.69 (s, 1H), 7.97 (s, 1H), 7.88 (s, 1H), 6.92 (m, 2H), 5.67 (s, 1H), 4.99 (m, 1H), 4.71 (s, 2H), 4.66 (s, 1H), 3.65-3.52 (m, 1H), 3.31-3.28 (m, 2H), 3.06-2.92 (m, 1H), 2.44 (m, 1H), 2.04-1.95 (m, 1H), 1.94-1.83 (m, 1H), 1.69 (m, 2H), 1.57 (m, 1H), 1.20 (s, 6H), 1.03 (m, 6H). LC-MS (ESI): m/z [M+H]⁺=510.32;

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=40: 60 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=65: 35 (v/v); Flow Rate: 18 mL/min, Wavelength: UV 270 nm and 330 nm, Prep-HPLC Equipment: Prep-HPLC-Gilson; Column temperature: 25° C.

### Example 255: cis-3-(3-((4-fluoro-1-methyl-2,2-dioxido-1,3-dihydrobenzo[c]isothiazol-5-yl)amino)-1H-pyrazol-5-yl)cyclopentyl isopropylcarbamate

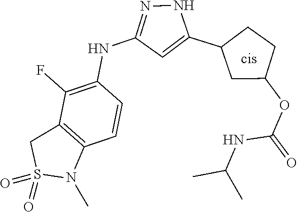 [SMILES: CC(C)NC(=O)OC1CCC(c2cc(Nc3ccc4c(c3F)CS(=O)(=O)N4C)n[nH]2)C1]

The titled compound was synthesized in the procedures similar to Example 203 in racemic form, which was further separated by Chiral Prep-HPLC to give:

Enantiomer 1 (Example 255a, 100% ee); Retention time: 4.68 min ¹H NMR (500 MHz, DMSO-d₆) δ 11.70 (s, 1H), 8.04 (s, 1H), 7.92 (s, 1H), 6.94 (d, J=7.0 Hz, 1H), 6.68 (d, J=8.7 Hz, 1H), 5.68 (s, 1H), 4.99 (s, 1H), 4.73 (s, 2H), 3.57 (d, J=6.7 Hz, 1H), 3.00 (d, J=13.9 Hz, 4H), 2.46-2.38 (m, 1H), 2.06-1.96 (m, 1H), 1.95-1.85 (m, 1H), 1.70 (s, 2H), 1.57 (d, J=9.9 Hz, 1H), 1.03 (d, J=6.1 Hz, 6H).

LC-MS (ESI): m/z [M+H]⁺=452.2.

Enantiomer 2 (Example 255b, 100% ee); Retention time: 6.311 min. ¹H NMR (500 MHz, DMSO-d₆) δ 11.70 (s, 1H), 8.04 (s, 1H), 7.92 (s, 1H), 6.94 (d, J=7.0 Hz, 1H), 6.68 (d, J=8.7 Hz, 1H), 5.68 (s, 1H), 4.99 (s, 1H), 4.73 (s, 2H), 3.57 (d, J=6.7 Hz, 1H), 3.00 (d, J=13.9 Hz, 4H), 2.46-2.38 (m, 1H), 2.06-1.96 (m, 1H), 1.95-1.85 (m, 1H), 1.70 (s, 2H), 1.57 (d, J=9.9 Hz, 1H), 1.03 (d, J=6.1 Hz, 6H). LC-MS (ESI): m/z [M+H]⁺=452.2.

Chiral analytical method: Column: I-Cellulose-5, 4.6 mm×250 mm, 5 μm; Mobile phase: A for MtBE (0.1% 2M NH₃ MeOH) and B for DCM:MeOH=50:50 (v/v); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 μm; Mobile phase: A for MtBE and B for DCM:MeOH=50:50 (v/v) (0.2% 2M NH₃ MeOH); Gradient: Mobile Phase A: Mobile Phase A: Mobile Phase B=50: 50 (v/v); Flow Rate: 20 mL/min, Wavelength: UV 200 nm and 270 nm, Prep-HPLC Equipment: Prep-HPLC-Gilson, Back pressure: 100 bar; Column temperature: 25° C.

### Example 256: cis-3-(3-((4-fluoro-1-methyl-2,2-dioxido-1,3-dihydrobenzo[c]isothiazol-5-yl)amino)-1H-pyrazol-5-yl)cyclopentyl propylcarbamate

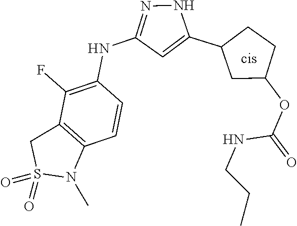 [SMILES: CCCNC(=O)OC1CCC(c2cc(Nc3ccc4c(c3F)CS(=O)(=O)N4C)n[nH]2)C1]

The titled compound was synthesized in the procedures similar to Example 203 in a racemic form, which was further separated by Chiral Prep-HPLC to give:

Enantiomer 1 (Example 256a, 100% ee); Retention time: 4.856 min. ¹H NMR (500 MHz, DMSO-d₆) δ=11.70 (s, 1H), 8.04 (s, 1H), 7.91 (s, 1H), 7.04 (t, J=5.5, 1H), 6.68 (d, J=8.7, 1H), 5.68 (s, 1H), 4.99 (s, 1H), 4.72 (s, 2H), 3.03 (m, 1H), 2.99 (s, 3H), 2.91 (m, 2H), 2.44 (m, 1H), 2.06-1.95 (m, 1H), 1.95-1.83 (m, 1H), 1.69 (m, 2H), 1.58 (m, 1H), 1.43-1.31 (m, 2H), 0.82 (t, J=7.4, 3H). LC-MS (ESI): m/z [M+H]⁺=452.32.

Enantiomer 2 (Example 256b, 100% ee); Retention time: 6.359 min. ¹H NMR (500 MHz, DMSO-d₆) δ=11.70 (s, 1H), 8.04 (s, 1H), 7.91 (s, 1H), 7.04 (s, 1H), 6.68 (d, J=5.6, 1H), 5.73-5.60 (m, 1H), 4.99 (s, 1H), 4.78-4.64 (m, 2H), 3.06-2.96 (m, 4H), 2.92 (s, 2H), 2.45 (m, 1H), 2.00 (m, 1H), 1.89 (m, 1H), 1.71 (m, 2H), 1.59 (m, 1H), 1.39 (m, 2H), 0.82 (m, 3H). LC-MS (ESI): m/z [M+H]⁺=452.33.

Chiral analytical method: Column: CHIRALPAK IE 4.6×250 mm 5 μm; Mobile phase: A for MtBE (0.1% 2M NH₃ MeOH) and B for MeOH: DCM=50:50 (v/v); Gradient: Mobile Phase A: Mobile Phase B=50: 50 (v/v); HPLC Equipment: HPLC-Agilent; Column temperature: 35° C.

Chiral Prep-HPLC Condition: CHIRALPAK IE 21.2 mm×250 mm, 5 μm; Mobile phase: A for MtBE and B for DCM:MeOH=50:50 (v/v) (0.2% 2 M NH₃. MeOH); Gradient: Mobile Phase A: Mobile Phase B=50: 50 (v/v); Flow Rate: 18 mL/min, Wavelength: UV 280 nm and 300 nm, Prep-HPLC Equipment: Prep-HPLC-Gilson; Column temperature: 25° C.

### Example 257: cis-3-(3-((4-fluoro-1-methyl-2,2-dioxido-1,3-dihydrobenzo[c]isothiazol-5-yl)amino)-1H-pyrazol-5-yl)cyclopentyl(1-methylcyclopropyl)carbamate

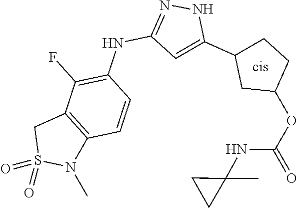 [SMILES: CN1c2ccc(Nc3cc(C4CCC(OC(=O)NC5(C)CC5)C4)[nH]n3)c(F)c2CS1(=O)=O]

The titled compound was synthesized in the procedures similar to Example 203. ¹H NMR (500 MHz, DMSO-d₆) δ 11.70 (s, 1H), 8.01 (s, 1H), 7.91 (s, 1H), 7.34 (s, 1H), 6.68 (d, J=8.7 Hz, 1H), 5.67 (s, 1H), 4.98 (s, 1H), 4.72 (s, 2H), 3.00 (s, 1H), 2.99 (s, 3H), 2.47-2.39 (m, 1H), 1.99 (d, J=9.2 Hz, 1H), 1.90 (d, J=20.2 Hz, 1H), 1.68 (s, 2H), 1.55 (s, 1H), 1.23 (s, 3H), 0.59 (s, 2H), 0.48-0.44 (m, 2H).

LC-MS (ESI): m/z [M+H]⁺=464.2.

### Example 258: cis-3-(3-((4-fluoro-1-methyl-2,2-dioxido-1,3-dihydrobenzo[c]isothiazol-5-yl)amino)-1H-pyrazol-5-yl)cyclopentyl((S)-sec-butyl)carbamate

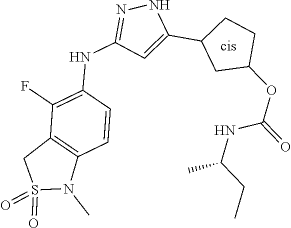 [SMILES: CC[C@H](C)NC(=O)OC1CCC(c2cc(Nc3ccc4c(c3F)CS(=O)(=O)N4C)n[nH]2)C1]

The titled compound was synthesized in the procedures similar to Example 203. ¹H NMR (500 MHz, DMSO-d₆) δ 11.70 (s, 1H), 8.04 (s, 1H), 7.91 (s, 1H), 6.88 (d, J=8.1 Hz, 1H), 6.68 (d, J=8.8 Hz, 1H), 5.68 (s, 1H), 4.99 (s, 1H), 4.72 (s, 2H), 3.38 (s, 1H), 3.04 (s, 1H), 2.99 (s, 3H), 2.47 (s, 1H), 1.99 (s, 1H), 1.90 (s, 1H), 1.69 (d, J=9.3 Hz, 2H), 1.58 (s, 1H), 1.35 (s, 2H), 1.00 (d, J=6.2 Hz, 3H), 0.80 (dd, J=13.0, 6.6 Hz, 3H). LC-MS (ESI): m/z [M+H]⁺=466.2

### Example 259: cis-3-(3-((4-fluoro-1-(oxetan-3-yl)-2,2-dioxido-1,3-dihydrobenzo[c]isothiazol-5-yl)amino)-1H-pyrazol-5-yl)cyclopentyl isopropylcarbamate

### Step 1:5-bromo-4-fluoro-1-(oxetan-3-yl)-1,3-dihydrobenzo[c]isothiazole 2,2-dioxide

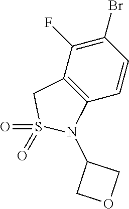 [SMILES: O=S1(=O)Cc2c(ccc(Br)c2F)N1C1COC1]

To a stirring solution of 5-bromo-4-fluoro-1,3-dihydrobenzo[c]isothiazole 2,2-dioxide (750 mg, 2.83 mmol) in toluene (20 mL) was added oxetan-3-ol (418.8 mg, 5.66 mmol) CMBP (2.07 g, 8.49 mmol). The solution was stirred for 90° C. for 16 h under N₂ atmosphere. The mixture was concentrated under reduced pressure. The residue was purified by silica gel column chromatography, eluting with PE/EtOAc (3:1) to afford the product (380 mg, 41.8%), LC-MS (ESI): m/z [M+H]⁺=321.9.

### Step 2:3-(3-((4-fluoro-1-(oxetan-3-yl)-2,2-dioxido-1,3-dihydrobenzo[c]isothiazol-5-yl)amino)-1H-pyrazol-5-yl)cyclopentyl isopropylcarbamate

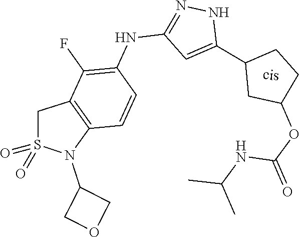 [SMILES: CC(C)NC(=O)OC1CCC(c2cc(Nc3ccc4c(c3F)CS(=O)(=O)N4C3COC3)n[nH]2)C1]

A mixture of 5-bromo-4-fluoro-1-(oxetan-3-yl)-1,3-dihydrobenzo[c]isothiazole 2,2-dioxide (150 mg, 0.46 mmol), cis-3-(3-amino-1H-pyrazol-5-yl)cyclopentyl isopropylcarbamate (118.2 mg, 0.46 mmol), Brettphos Pd G3 (41.4 mg, 0.046 mmol), K₂CO₃ (206 mg, 1.5 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product. The residue was purified by prep HPLC (Waters XSelect C18: RD-CO-094 column, eluting with a gradient of acetonitrile/water containing 0.1% FA, 20%-40%) to afford the product (28.5 mg, 37.2%) in racemic form, which was further separated by Chiral Prep-HPLC to give:

Enantiomer 1 (Example 259a, 100% ee); Retention time: 6.05 min. ¹H NMR (500 MHz, DMSO-d₆) δ 11.91 (s, 1H), 8.15 (s, 1H), 7.96 (s, 1H), 6.94 (d, J=7.5 Hz, 1H), 6.68 (d, J=8.7 Hz, 1H), 5.71 (s, 1H), 5.02-4.96 (m, 3H), 4.93-4.87 (m, 3H), 4.82 (s, 2H), 3.64-3.52 (m, 1H), 3.06-2.89 (m, 1H), 2.47-2.40 (m, 1H), 2.06-1.98 (m, 1H), 1.96-1.86 (m, 1H), 1.75-1.65 (m, 2H), 1.63-1.56 (m, 1H), 1.08-0.95 (m, 6H). LC-MS (ESI): m/z [M+H]⁺=494.2.

Enantiomer 2 (Example 259b, 95.4% ee); Retention time: 7.57 min. ¹H NMR (500 MHz, DMSO-d₆) δ 11.91 (s, 1H), 8.15 (s, 1H), 7.96 (s, 1H), 6.94 (d, J=7.5 Hz, 1H), 6.68 (d, J=8.7 Hz, 1H), 5.71 (s, 1H), 5.02-4.96 (m, 3H), 4.93-4.87 (m, 3H), 4.82 (s, 2H), 3.64-3.52 (m, 1H), 3.06-2.89 (m, 1H), 2.47-2.40 (m, 1H), 2.06-1.98 (m, 1H), 1.96-1.86 (m, 1H), 1.75-1.65 (m, 2H), 1.63-1.56 (m, 1H), 1.08-0.95 (m, 6H). LC-MS (ESI): m/z [M+H]⁺=494.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=60: 40 (v/v); HPLC Equipment: HPLC-Agilent, Back pressure: 100 bar; Column temperature: RT.

### Example 260: cis-3-(3-((4-fluoro-1-(oxetan-3-yl)-2,2-dioxido-1,3-dihydrobenzo[c]isothiazol-5-yl)amino)-1H-pyrazol-5-yl)cyclopentyl propylcarbamate

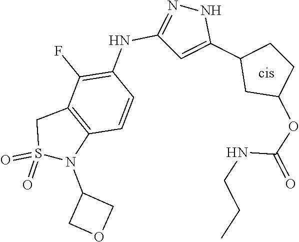 [SMILES: CCCNC(=O)OC1CCC(c2cc(Nc3ccc4c(c3F)CS(=O)(=O)N4C3COC3)n[nH]2)C1]

The titled compound was synthesized in the procedures similar to Example 259 in racemic form, which was further separated by Chiral Prep-HPLC to give:

Enantiomer 1 (Example 260a, 100% ee); Retention time: 5.37 min. ¹H NMR (500 MHz, DMSO-d₆) δ 11.78 (s, 1H), 8.02 (s, 2H), 7.04 (t, J=5.4 Hz, 1H), 6.66 (d, J=8.8 Hz, 1H), 5.70 (s, 1H), 5.02-4.96 (m, 2H), 4.92-4.86 (m, 3H), 4.81 (s, 2H), 3.10-2.98 (m, 1H), 2.91 (dd, J=13.1, 6.6 Hz, 2H), 2.48-2.37 (m, 1H), 2.05-1.97 (m, 1H), 1.95-1.81 (m, 1H), 1.78-1.64 (m, 2H), 1.63-1.53 (m, 1H), 1.45-1.30 (m, 2H), 0.81 (t, J=7.4 Hz, 3H). LC-MS (ESI): m/z [M+H]⁺=494.2.

Enantiomer 2 (Example 260b, 100% ee); Retention time: 6.10 min. ¹H NMR (500 MHz, DMSO-d₆) δ 11.78 (s, 1H), 8.02 (s, 2H), 7.04 (t, J=5.4 Hz, 1H), 6.66 (d, J=8.8 Hz, 1H), 5.70 (s, 1H), 5.02-4.96 (m, 2H), 4.92-4.86 (m, 3H), 4.81 (s, 2H), 3.10-2.98 (m, 1H), 2.91 (dd, J=13.1, 6.6 Hz, 2H), 2.48-2.37 (m, 1H), 2.05-1.97 (m, 1H), 1.95-1.81 (m, 1H), 1.78-1.64 (m, 2H), 1.63-1.53 (m, 1H), 1.45-1.30 (m, 2H), 0.81 (t, J=7.4 Hz, 3H). LC-MS (ESI): m/z [M+H]⁺=494.2.

Chiral analytical method: Column: CHIRALPAK IE 4.6 mm×250 mm, 5 μm; Mobile phase: A for MtBE (0.1% 2M NH₃ MeOH) (%) and B for MeOH/DCM (50:50) (%); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for MeOH/DCM (50:50) (%); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 261: cis-3-(3-((4-fluoro-1-(oxetan-3-yl)-2,2-dioxido-1,3-dihydrobenzo[c]isothiazol-5-yl)amino)-1H-pyrazol-5-yl)cyclopentyl((S)-sec-butyl)carbamate

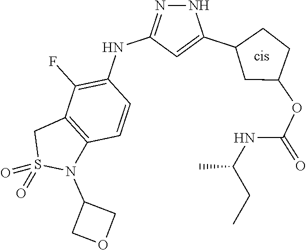 [SMILES: CC[C@H](C)NC(=O)OC1CCC(c2cc(Nc3ccc4c(c3F)CS(=O)(=O)N4C3COC3)n[nH]2)C1]

The titled compound was synthesized in the procedures similar to Example 259 in a racemic form, which was further separated by Chiral Prep-HPLC to give:

Enantiomer 1 (Example 261a, 100% ee); Retention time: 5.08 min. ¹H NMR (500 MHz, DMSO-d₆) δ 11.82 (s, 1H), 8.03 (d, J=32.1 Hz, 2H), 6.88 (d, J=8.1 Hz, 1H), 6.67 (d, J=8.8 Hz, 1H), 5.70 (s, 1H), 5.02-4.96 (m, 3H), 4.92-4.86 (m, 3H), 4.82 (s, 2H), 3.45-3.30 (m, 1H), 3.08-2.98 (m, 1H), 2.46-2.37 (m, 1H), 2.08-1.97 (m, 1H), 1.95-1.81 (m, 1H), 1.81-1.64 (m, 2H), 1.63-1.53 (m, 1H), 1.39-1.26 (m, 2H), 1.04-0.96 (M, 3H), 0.86-0.76 (m, 3H). LC-MS (ESI): m/z [M+H]⁺=508.2.

Enantiomer 2 (Example 261b, 100% ee); Retention time: 6.11 min. ¹H NMR (500 MHz, DMSO-d₆) δ 11.82 (s, 1H), 8.03 (d, J=32.1 Hz, 2H), 6.88 (d, J=8.1 Hz, 1H), 6.67 (d, J=8.8 Hz, 1H), 5.70 (s, 1H), 5.02-4.96 (m, 3H), 4.92-4.86 (m, 3H), 4.82 (s, 2H), 3.45-3.30 (m, 1H), 3.08-2.98 (m, 1H), 2.46-2.37 (m, 1H), 2.08-1.97 (m, 1H), 1.95-1.81 (m, 1H), 1.81-1.64 (m, 2H), 1.63-1.53 (m, 1H), 1.39-1.26 (m, 2H), 1.04-0.96 (M, 3H), 0.86-0.76 (m, 3H). LC-MS (ESI): m/z [M+H]⁺=508.2.

Chiral analytical method: Column: CHIRALPAK IE 4.6 mm×250 mm, 5 μm; Mobile phase: A for MtBE (0.1% 2M NH₃ MeOH) (%) and B for MeOH/DCM (50:50) (%); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE (0.2% 2M NH₃ MeOH) (%) and B for MeOH/DCM (50:50) (%); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 262: cis-3-(3-((4-fluoro-1-(oxetan-3-yl)-2,2-dioxido-1,3-dihydrobenzo[c]isothiazol-5-yl)amino)-1H-pyrazol-5-yl)cyclopentyl(1-methylcyclopropyl)carbamate

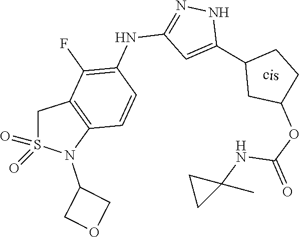 [SMILES: CC1(NC(=O)OC2CCC(c3cc(Nc4ccc5c(c4F)CS(=O)(=O)N5C4COC4)n[nH]3)C2)CC1]

The titled compound was synthesized in the procedures similar to Example 259. ¹H NMR (500 MHz, DMSO-d₆) δ 11.75 (s, 1H), 8.02 (s, 2H), 7.33 (s, 1H), 6.66 (d, J=8.8 Hz, 1H), 5.69 (s, 1H), 5.02-4.97 (m, 3H), 4.92-4.85 (m, 3H), 4.81 (s, 2H), 3.44-3.28 (m, 2H), 3.08-2.96 (m, 1H), 2.48-2.38 (m, 1H), 2.05-1.97 (m, 1H), 1.95-1.81 (m, 1H), 1.78-1.64 (m, 2H), 1.63-1.53 (m, 1H), 1.23 (s, 3H), 0.61-0.57 (m, 2H), 0.49-0.45 (m, 2H). LC-MS (ESI): m/z [M+H]⁺=506.2.

### Example 263: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl isopropylcarbamate

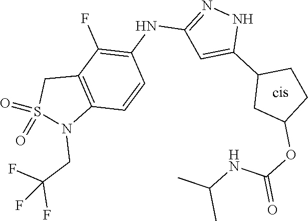 [SMILES: CC(C)NC(=O)OC1CCC(c2cc(Nc3ccc4c(c3F)CS(=O)(=O)N4CC(F)(F)F)n[nH]2)C1]

The titled compound was synthesized in the procedures similar to Example 259 in a racemic form, which was further separated by Chiral Prep-HPLC to give:

Enantiomer 1 (Example 263a, 100% ee); Retention time: 5.595 min. ¹H NMR (500 MHz, DMSO-d₆) δ 11.74 (s, 1H), 8.05 (d, J=20.4 Hz, 2H), 6.94 (d, J=7.2 Hz, 1H), 6.83 (d, J=8.8 Hz, 1H), 5.70 (s, 1H), 4.99 (s, 1H), 4.86 (s, 2H), 4.43 (q, J=9.2 Hz, 2H), 3.57 (d, J=6.2 Hz, 1H), 3.02 (s, 1H), 2.46-2.41 (m, 1H), 2.00 (d, J=8.5 Hz, 1H), 1.90 (d, J=6.0 Hz, 1H), 1.70 (t, J=10.4 Hz, 2H), 1.58 (s, 1H), 1.03 (d, J=6.2 Hz, 6H). LC-MS (ESI): m/z [M+H]⁺=520.2.

Enantiomer 2 (Example 263b, 100% ee); Retention time: 7.133 min. ¹H NMR (500 MHz, DMSO-d₆) δ 11.74 (s, 1H), 8.05 (d, J=20.4 Hz, 2H), 6.94 (d, J=7.2 Hz, 1H), 6.83 (d, J=8.8 Hz, 1H), 5.70 (s, 1H), 4.99 (s, 1H), 4.86 (s, 2H), 4.43 (q, J=9.2 Hz, 2H), 3.57 (d, J=6.2 Hz, 1H), 3.02 (s, 1H), 2.46-2.41 (m, 1H), 2.00 (d, J=8.5 Hz, 1H), 1.90 (d, J=6.0 Hz, 1H), 1.70 (t, J=10.4 Hz, 2H), 1.58 (s, 1H), 1.03 (d, J=6.2 Hz, 6H). LC-MS (ESI): m/z [M+H]⁺=520.2.

Chiral analytical method: Column: I-Cellulose-5, 4.6 mm×250 mm, 5 μm; Mobile phase: A for Hexane and B for EtOH (0.2% 2M NH₃ MeOH); Gradient: Mobile Phase A: Mobile Phase B=30: 70 (v/v); HPLC Equipment: HPLC-Agilent, Back pressure: 100 bar; Column temperature: 35° C.

Chiral Prep-HPLC Condition: Column: IE, 21.2 mm×250 mm, 5 μm; Mobile phase: A for Hexane and B for EtOH (0.2% 2M NH₃ MeOH); Gradient: Mobile Phase A: Mobile Phase B=30: 70 (v/v), Flow Rate: 1.0 mL/min, Wavelength: UV 200 nm and 270 nm, Prep-HPLC Equipment: Prep-HPLC-Gilson, Back pressure: 100 bar; Column temperature: RT.

### Example 264: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl(1-methylcyclopropyl)carbamate

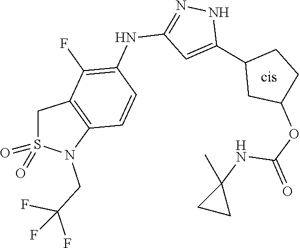 [SMILES: CC1(NC(=O)OC2CCC(c3cc(Nc4ccc5c(c4F)CS(=O)(=O)N5CC(F)(F)F)n[nH]3)C2)CC1]

The titled compound was synthesized in the procedures similar to Example 259 in a racemic form, which was further separated by Chiral Prep-HPLC to give:

Enantiomer 1 (Example 264a, 100% ee); Retention time: 5.238 min. ¹H NMR (500 MHz, DMSO-d₆) δ 11.83 (s, 1H), 8.15-7.96 (m, 2H), 7.33 (s, 1H), 6.82 (s, 1H), 5.69 (s, 1H), 4.94 (d, J=33.8 Hz, 1H), 4.91 (s, 2H), 4.42 (s, 2H), 3.02 (s, 1H), 2.47 (d, J=27.8 Hz, 8H), 1.96 (d, J=31.5 Hz, 1H), 1.88 (s, 1H), 1.68 (s, 2H), 1.55 (s, 1H), 1.23 (s, 3H), 0.55 (d, J=37.0 Hz, 2H), 0.47 (s, 2H). LC-MS (ESI): m/z [M+H]⁺=532.3.

Enantiomer 2 (Example 264b, 98.84% ee); Retention time: 6.388 min. ¹H NMR (500 MHz, DMSO-d₆) δ 11.83 (s, 1H), 8.15-7.96 (m, 2H), 7.33 (s, 1H), 6.82 (s, 1H), 5.69 (s, 1H), 4.94 (d, J=33.8 Hz, 1H), 4.91 (s, 2H), 4.42 (s, 2H), 3.02 (s, 1H), 2.47 (d, J=27.8 Hz, 8H), 1.96 (d, J=31.5 Hz, 1H), 1.88 (s, 1H), 1.68 (s, 2H), 1.55 (s, 1H), 1.23 (s, 3H), 0.55 (d, J=37.0 Hz, 2H), 0.47 (s, 2H). LC-MS (ESI): m/z [M+H]⁺=532.3.

Chiral analytical method: Column: I-Cellulose-5, 4.6 mm×250 mm, 5 μm; Mobile phase: A for Hexane and B for EtOH (0.2% 2M NH₃ MeOH); Gradient: Mobile Phase A: Mobile Phase B=30: 70 (v/v); HPLC Equipment: HPLC-Agilent, Back pressure: 100 bar; Column temperature: 35° C.

Chiral Prep-HPLC Condition: Column: IE, 21.2 mm×250 mm, 5 μm; Mobile phase: A for Hexane and B for EtOH (0.2% 2M NH₃ MeOH); Gradient: Mobile Phase A: Mobile Phase B=30: 70 (v/v), Flow Rate: 1.0 mL/min, Wavelength: UV 200 nm and 270 nm, Prep-HPLC Equipment: Prep-HPLC-Gilson, Back pressure: 100 bar; Column temperature: RT.

### Example 265: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl((S)-sec-butyl)carbamate

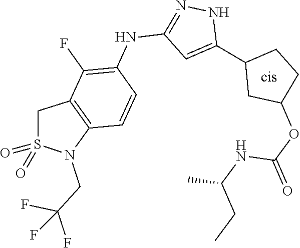 [SMILES: CC[C@H](C)NC(=O)OC1CCC(c2cc(Nc3ccc4c(c3F)CS(=O)(=O)N4CC(F)(F)F)n[nH]2)C1]

The titled compound was synthesized in the procedures similar to Example 259 in a racemic form, which was further separated by Chiral Prep-HPLC to give:

Enantiomer 1 (Example 265a, 100% ee); Retention time: 5.215 min. ¹H NMR (500 MHz, DMSO-d₆) δ 11.77 (s, 1H), 8.04 (d, J=9.1 Hz, 1H), 6.86 (dd, J=21.2, 8.5 Hz, 1H), 5.70 (s, 1H), 4.99 (s, 1H), 4.86 (s, 1H), 4.43 (q, J=9.3 Hz, 1H), 3.38 (dt, J=14.1, 6.9 Hz, 1H), 3.04 (dd, J=16.8, 8.4 Hz, 1H), 2.45 (dd, J=13.5, 6.9 Hz, OH), 2.01 (dd, J=15.7, 7.2 Hz, 1H), 1.95-1.84 (m, 1H), 1.79-1.64 (m, 1H), 1.64-1.51 (m, 1H), 1.43-1.27 (m, 1H), 1.09-0.92 (m, 2H), 0.81 (dd, J=13.7, 6.7 Hz, 2H). LC-MS (ESI): m/z [M+H]⁺=534.3.

Enantiomer 2 (Example 265b, 100% ee); Retention time: 7.121 min. ¹H NMR (500 MHz, DMSO-d₆) δ 11.77 (s, 1H), 8.04 (d, J=9.1 Hz, 1H), 6.86 (dd, J=21.2, 8.5 Hz, 1H), 5.70 (s, 1H), 4.99 (s, 1H), 4.86 (s, 1H), 4.43 (q, J=9.3 Hz, 1H), 3.38 (dt, J=14.1, 6.9 Hz, 1H), 3.04 (dd, J=16.8, 8.4 Hz, 1H), 2.45 (dd, J=13.5, 6.9 Hz, OH), 2.01 (dd, J=15.7, 7.2 Hz, 1H), 1.95-1.84 (m, 1H), 1.79-1.64 (m, 1H), 1.64-1.51 (m, 1H), 1.43-1.27 (m, 1H), 1.09-0.92 (m, 2H), 0.81 (dd, J=13.7, 6.7 Hz, 2H). LC-MS (ESI): m/z [M+H]⁺=534.3.

Chiral analytical method: Column: I-Cellulose-5, 4.6 mm×250 mm, 5 μm; Mobile phase: A for Hexane and B for EtOH (0.2% 2M NH₃ MeOH); Gradient: Mobile Phase A: Mobile Phase B=30: 70 (v/v); HPLC Equipment: HPLC-Agilent, Back pressure: 100 bar; Column temperature: 35° C.

Chiral Prep-HPLC Condition: Column: IE, 21.2 mm×250 mm, 5 μm; Mobile phase: A for Hexane and B for EtOH (0.2% 2M NH₃ MeOH); Gradient: Mobile Phase A: Mobile Phase B=30: 70 (v/v), Flow Rate: 1.0 mL/min, Wavelength: UV 200 nm and 270 nm, Prep-HPLC Equipment: Prep-HPLC-Gilson, Back pressure: 100 bar; Column temperature: RT.

### Example 266: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl propylcarbamate

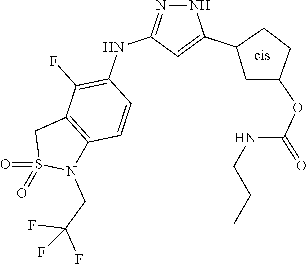 [SMILES: CCCNC(=O)OC1CCC(c2cc(Nc3ccc4c(c3F)CS(=O)(=O)N4CC(F)(F)F)n[nH]2)C1]

The titled compound was synthesized in the procedures similar to Example 259 in racemic form, which was further separated by Chiral Prep-HPLC to give:

Enantiomer 1 (Example 266a, 100% ee); Retention time: 4.825 min. ¹H NMR (500 MHz, DMSO-d₆) δ 11.74 (s, 1H), 8.05 (d, J=18.3 Hz, 2H), 7.04 (t, J=5.5 Hz, 1H), 6.83 (d, J=8.8 Hz, 1H), 5.70 (s, 1H), 5.08-4.98 (m, 1H), 4.86 (s, 2H), 4.43 (q, J=9.4 Hz, 2H), 3.11-2.97 (m, 1H), 2.91 (dd, J=13.1, 6.6 Hz, 2H), 2.44 (dd, J=14.0, 7.2 Hz, 1H), 2.04-1.95 (m, 1H), 1.94-1.84 (m, 1H), 1.69 (dt, J=18.4, 8.8 Hz, 3H), 1.61-1.55 (m, 1H), 1.38 (dq, J=14.4, 7.2 Hz, 2H), 0.82 (t, J=7.4 Hz, 3H). LC-MS (ESI): m/z [M+H]⁺=520.2.

Enantiomer 2 (Example 266b, 98.18% ee); Retention time: 5.487 min. ¹H NMR (500 MHz, DMSO-d₆) δ 11.74 (s, 1H), 8.05 (d, J=18.3 Hz, 2H), 7.04 (t, J=5.5 Hz, 1H), 6.83 (d, J=8.8 Hz, 1H), 5.70 (s, 1H), 5.08-4.98 (m, 1H), 4.86 (s, 2H), 4.43 (q, J=9.4 Hz, 2H), 3.11-2.97 (m, 1H), 2.91 (dd, J=13.1, 6.6 Hz, 2H), 2.44 (dd, J=14.0, 7.2 Hz, 1H), 2.04-1.95 (m, 1H), 1.94-1.84 (m, 1H), 1.69 (dt, J=18.4, 8.8 Hz, 3H), 1.61-1.55 (m, 1H), 1.38 (dq, J=14.4, 7.2 Hz, 2H), 0.82 (t, J=7.4 Hz, 3H). LC-MS (ESI): m/z [M+H]⁺=520.2.

Chiral analytical method: Column: I-Cellulose-5, 4.6 mm×250 mm, 5 μm; Mobile phase: A for Hexane and B for EtOH (0.2% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: Column: IE, 21.2 mm×250 mm, 5 μm; Mobile phase: A for Hexane and B for EtOH (0.2% 2M NH₃ MeOH); Gradient: Mobile Phase A: Mobile Phase B=20: 80 (v/v), Flow Rate: 1.0 mL/min, Wavelength: UV 200 nm and 270 nm, Prep-HPLC Equipment: Prep-HPLC-Gilson, Back pressure: 100 bar; Column temperature: RT.

### Example 267: cis-3-(3-((4-fluoro-1-(methyl-d3)-2,2-dioxido-1,3-dihydrobenzo[c]isothiazol-5-yl)amino)-1H-pyrazol-5-yl)cyclopentyl isopropylcarbamate

### Step 1:5-bromo-4-fluoro-1-(methyl-d3)-1,3-dihydrobenzo[c]isothiazole 2,2-dioxide

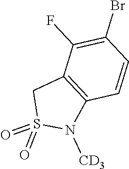 [SMILES: [2H]C([2H])([2H])N1c2ccc(Br)c(F)c2CS1(=O)=O]

To a stirring solution of 5-bromo-4-fluoro-1,3-dihydrobenzo[c]isothiazole 2,2-dioxide (750 mg, 2.83 mmol) in DMF (20 mL) was added K₂CO₃ (781 mg, 5.66 mmol), CD₃I (820.7 mg, 5.66 mmol). The solution was stirred for 3 h at RT. The mixture was concentrated under reduced pressure. The residue was purified by silica gel column chromatography, eluting with PE/EtOAc (4:1) to afford the product (700 mg, 87.3%), LC-MS (ESI): m/z [M+H]⁺=282.9.

### Step 2: cis-3-(3-((4-fluoro-1-(methyl-d3)-2,2-dioxido-1,3-dihydrobenzo[c]isothiazol-5-yl)amino)-1H-pyrazol-5-yl)cyclopentyl isopropylcarbamate

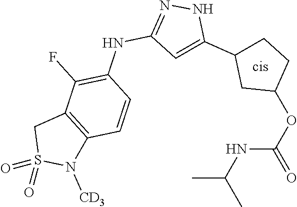 [SMILES: [2H]C([2H])([2H])N1c2ccc(Nc3cc(C4CCC(OC(=O)NC(C)C)C4)[nH]n3)c(F)c2CS1(=O)=O]

A mixture of 5-bromo-4-fluoro-1-(methyl-d3)-1,3-dihydrobenzo[c]isothiazole 2,2-dioxide (150 mg, 0.53 mmol), cis-3-(3-amino-1H-pyrazol-5-yl)cyclopentyl isopropylcarbamate (134.6 mg, 0.53 mmol), Brettphos Pd G3 (47.7 mg, 0.053 mmol), K₂CO₃ (219.4 mg, 1.59 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product. The residue was purified by prep HPLC (Waters XSelect C18: RD-CO-094 column, eluting with a gradient of acetonitrile/water containing 0.1% FA, 20%-40%) to afford the product in racemic form, which was further separated by Chiral Prep-HPLC to give:

Enantiomer 1 (Example 267a, 100% ee); Retention time: 4.70 min. ¹H NMR (500 MHz, DMSO-d₆) δ 11.70 (s, 1H), 8.04 (s, 1H), 7.92 (s, 1H), 6.94 (d, J=7.4 Hz, 1H), 6.68 (d, J=8.7 Hz, 1H), 5.68 (s, 1H), 4.99 (s, 1H), 4.72 (s, 2H), 3.65-3.51 (m, 1H), 3.10-2.96 (m, 1H), 2.50-2.44 (m, 1H), 2.03-1.96 (m, 1H), 1.95-1.85 (m, 1H), 1.78-1.65 (m, 2H), 1.64-1.55 (m, 1H), 1.03 (d, J=6.3 Hz, 6H).

LC-MS (ESI): m/z [M+H]⁺=455.2.

Enantiomer 2 (Example 267b, 100% ee); Retention time: 6.36 min. ¹H NMR (500 MHz, DMSO-d₆) δ 11.70 (s, 1H), 8.04 (s, 1H), 7.92 (s, 1H), 6.94 (d, J=7.4 Hz, 1H), 6.68 (d, J=8.7 Hz, 1H), 5.68 (s, 1H), 4.99 (s, 1H), 4.72 (s, 2H), 3.65-3.51 (m, 1H), 3.10-2.96 (m, 1H), 2.50-2.44 (m, 1H), 2.03-1.96 (m, 1H), 1.95-1.85 (m, 1H), 1.78-1.65 (m, 2H), 1.64-1.55 (m, 1H), 1.03 (d, J=6.3 Hz, 6H).

LC-MS (ESI): m/z [M+H]⁺=455.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=50: 50 (v/v); Flow Rate: 18 mL/min, Wave Length: UV 270 nm and 320 nm, Prep-HPLC Equipment: Prep-HPLC-Gilson, Back pressure: 100 bar; Column temperature: 25° C.

### Example 268: cis-3-(3-((4-fluoro-1-(methyl-d3)-2,2-dioxido-1,3-dihydrobenzo[c]isothiazol-5-yl)amino)-1H-pyrazol-5-yl)cyclopentyl propylcarbamate

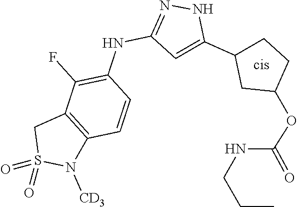 [SMILES: [2H]C([2H])([2H])N1c2ccc(Nc3cc(C4CCC(OC(=O)NCCC)C4)[nH]n3)c(F)c2CS1(=O)=O]

The titled compound was synthesized in the procedures similar to Example 267 in racemic form, which was further separated by Chiral Prep-HPLC to give:

Enantiomer 1 (Example 268a, 100% ee); Retention time: 4.63 min. ¹H NMR (500 MHz, DMSO-d₆) δ 11.70 (s, 1H), 8.04 (t, J=8.9 Hz, 1H), 7.91 (s, 1H), 7.04 (s, 1H), 6.68 (d, J=8.7 Hz, 1H), 5.68 (d, J=1.9 Hz, 1H), 5.02-4.92 (m, 1H), 4.72 (s, 2H), 3.12-2.96 (m, 1H), 2.95-2.88 (m, 2H), 2.45-2.37 (m, 1H), 2.07-1.97 (m, 1H), 1.97-1.86 (m, 1H), 1.79-1.66 (m, 2H), 1.63-1.53 (m, 1H), 1.42-1.34 (m, 2H), 0.82 (t, J=7.4 Hz, 3H). LC-MS (ESI): m/z [M+H]⁺=455.2.

Enantiomer 2 (Example 268b, 95.4% ee); Retention time: 6.00 min. ¹H NMR (500 MHz, DMSO-d₆) δ 11.70 (s, 1H), 8.04 (t, J=8.9 Hz, 1H), 7.91 (s, 1H), 7.04 (s, 1H), 6.68 (d, J=8.7 Hz, 1H), 5.68 (d, J=1.9 Hz, 1H), 5.02-4.92 (m, 1H), 4.72 (s, 2H), 3.12-2.96 (m, 1H), 2.95-2.88 (m, 2H), 2.45-2.37 (m, 1H), 2.07-1.97 (m, 1H), 1.97-1.86 (m, 1H), 1.79-1.66 (m, 2H), 1.63-1.53 (m, 1H), 1.42-1.34 (m, 2H), 0.82 (t, J=7.4 Hz, 3H). LC-MS (ESI): m/z [M+H]⁺=455.2.

Chiral analytical method: Column: CHIRALPAK IE 4.6 mm×250 mm, 5 μm; Mobile phase: A for MtBE (0.1% 2M NH₃ MeOH) and B for DCM: MeOH; Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM: MeOH (0.2% 2M NH₃ MeOH); Gradient: Mobile Phase A: Mobile Phase B=50: 50 (v/v); HPLC Equipment: HPLC-Agilent, Back pressure: 100 bar; Column temperature: RT.

### Example 269: cis-3-(3-((4-fluoro-1-(methyl-d3)-2,2-dioxido-1,3-dihydrobenzo[c]isothiazol-5-yl)amino)-1H-pyrazol-5-yl)cyclopentyl((S)-sec-butyl)carbamate

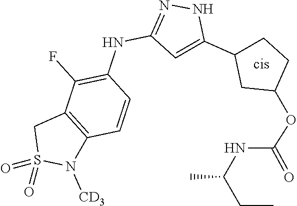 [SMILES: [2H]C([2H])([2H])N1c2ccc(Nc3cc(C4CCC(OC(=O)N[C@@H](C)CC)C4)[nH]n3)c(F)c2CS1(=O)=O]

The titled compound was synthesized in the procedures similar to Example 267. ¹H NMR (500 MHz, DMSO-d₆) δ 11.70 (s, 1H), 8.04 (s, 1H), 7.91 (s, 1H), 6.88 (d, J=8.2 Hz, 1H), 6.68 (d, J=8.7 Hz, 1H), 5.68 (s, 1H), 4.99 (s, 1H), 4.72 (s, 2H), 3.44-3.34 (m, 1H), 3.08-2.94 (m, 1H), 2.50-2.44 (m, 1H), 2.06-1.94 (m, 1H), 1.95-1.85 (m, 1H), 1.77-1.65 (m, 2H), 1.65-1.55 (m, 1H), 1.40-1.30 (m, 2H), 1.06-0.94 (m, 3H), 0.84-0.78 (m, 3H). LC-MS (ESI): m/z [M+H]⁺=469.2.

### Example 270: cis-3-(3-((4-fluoro-1-(methyl-d3)-2,2-dioxido-1,3-dihydrobenzo[c]isothiazol-5-yl)amino)-1H-pyrazol-5-yl)cyclopentyl(1-methylcyclopropyl)carbamate

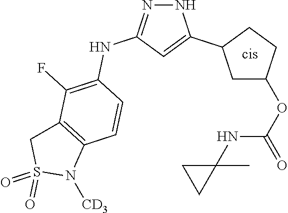 [SMILES: [2H]C([2H])([2H])N1c2ccc(Nc3cc(C4CCC(OC(=O)NC5(C)CC5)C4)[nH]n3)c(F)c2CS1(=O)=O]

The titled compound was synthesized in the procedures similar to Example 267. ¹H NMR (500 MHz, DMSO-d₆) δ 11.69 (s, 1H), 8.02 (s, 1H), 7.91 (s, 1H), 7.34 (s, 1H), 6.68 (d, J=8.7 Hz, 1H), 5.67 (s, 1H), 4.98 (s, 1H), 4.72 (s, 2H), 3.10-2.95 (m, 1H), 2.50-2.45 (m, 1H), 2.06-1.94 (m, 1H), 1.95-1.85 (m, 1H), 1.77-1.65 (m, 2H), 1.65-1.55 (m, 1H), 1.23 (s, 3H), 0.61-0.55 (m, 2H), 0.48-0.45 (m, 2H). LC-MS (ESI): m/z [M+H]⁺=467.2.

### Example 271: cis-3-(3-((4-fluoro-1-(2-hydroxyethyl)-2,2-dioxido-1,3-dihydrobenzo[c]isothiazol-5-yl)amino-1H-pyrazol-5-yl)cyclopentyl isopropylcarbamate

### Step 1:5-bromo-1-(2-((tert-butyldimethylsilyl)oxy) ethyl)-4-fluoro-1,3-dihydrobenzo[c]isothiazole 2,2-dioxide

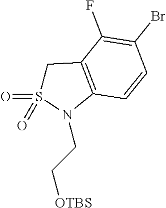 [SMILES: CC(C)(C)[Si](C)(C)OCCN1c2ccc(Br)c(F)c2CS1(=O)=O]

To a stirring solution of 5-bromo-4-fluoro-1,3-dihydrobenzo[c]isothiazole 2,2-dioxide (750 mg, 2.83 mmol) in DMF (20 mL) was added K₂CO₃ (781 mg, 5.66 mmol) (2-bromoethoxy) (tert-butyl)dimethylsilane (1.34 g, 5.66 mmol). The solution was stirred for 3 h at 50° C. The mixture was concentrated under reduced pressure. The residue was purified by silica gel column chromatography, eluting with PE/EtOAc (4:1) to afford the product (655 mg, 75%), LC-MS (ESI): m/z [M+Na]⁺=446.3.

### Step 2: cis-3-(3-((1-(2-((tert-butyldimethylsilyl)oxy) ethyl)-4-fluoro-2,2-dioxido-1,3-dihydrobenzo[c]isothiazol-5-yl)amino)-1H-pyrazol-5-yl)cyclopentyl isopropylcarbamate

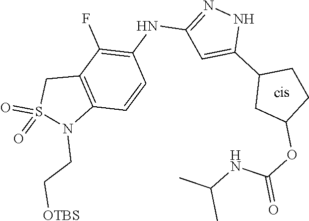 [SMILES: CC(C)NC(=O)OC1CCC(c2cc(Nc3ccc4c(c3F)CS(=O)(=O)N4CCO[Si](C)(C)C(C)(C)C)n[nH]2)C1]

A mixture of 5-bromo-1-(2-((tert-butyldimethylsilyl)oxy) ethyl)-4-fluoro-1,3-dihydrobenzo[c]isothiazole 2,2-dioxide (150 mg, 0.35 mmol), cis-3-(3-amino-1H-pyrazol-5-yl)cyclopentyl isopropylcarbamate (118.2 mg, 0.46 mmol), Brettphos Pd G3 (41.4 mg, 0.046 mmol), K₂CO₃ (145 mg, 1.05 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product 250 mg.

### Step 3: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl isopropylcarbamate

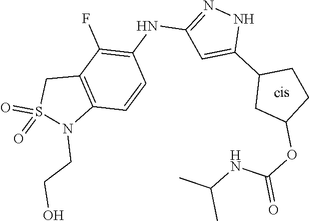 [SMILES: CC(C)NC(=O)OC1CCC(c2cc(Nc3ccc4c(c3F)CS(=O)(=O)N4CCO)n[nH]2)C1]

To a stirring solution of 3-(3-((1-(2-((tert-butyldimethylsilyl)oxy) ethyl)-4-fluoro-2,2-dioxido-1,3-dihydrobenzo[c]isothiazol-5-yl)amino)-1H-pyrazol-5-yl)cyclopentyl isopropylcarbamate (250 mg, 0.42 mmol) in DCM (20 mL) was added TFA (5 mL), The solution was stirred for 12 h at RT. The mixture was concentrated under reduced pressure. The residue was purified by silica gel column chromatography, eluting with EA/PE (10% to 100%) to afford the product. The residue was purified by prep HPLC (Waters XSelect C18: RD-CO-094 column, eluting with a gradient of acetonitrile/water containing 0.1% FA, 20%-40%) to afford the product (41.3 mg, 38.9%) in racemic form, which was further separated by Chiral Prep-HPLC to give:

Enantiomer 1 (Example 271a, 100% ee); Retention time: 6.03 min. ¹H NMR (500 MHz, DMSO-d₆) δ 11.69 (s, 1H), 8.00 (t, J=8.2 Hz, 1H), 7.89 (s, 1H), 6.94 (d, J=7.5 Hz, 1H), 6.76 (d, J=8.8 Hz, 1H), 5.67 (s, 1H), 5.03-4.93 (m, 2H), 4.69 (s, 2H), 3.66-3.60 (m, 2H), 3.59-3.47 (m, 3H), 3.09-2.95 (m, 1H), 2.46-2.38 (m, 2H), 2.05-1.96 (m, 1H), 1.96-1.85 (m, 1H), 1.75-1.65 (m, 2H), 1.58-1.52 (m, 1H), 1.03 (d, J=6.2 Hz, 6H). LC-MS (ESI): m/z [M+H]⁺=482.2.

Enantiomer 2 (Example 271b, 100% ee); Retention time: 8.25 min. ¹H NMR (500 MHz, DMSO-d₆) δ 11.69 (s, 1H), 8.00 (t, J=8.2 Hz, 1H), 7.89 (s, 1H), 6.94 (d, J=7.5 Hz, 1H), 6.76 (d, J=8.8 Hz, 1H), 5.67 (s, 1H), 5.03-4.93 (m, 2H), 4.69 (s, 2H), 3.66-3.60 (m, 2H), 3.59-3.47 (m, 3H), 3.09-2.95 (m, 1H), 2.46-2.38 (m, 2H), 2.05-1.96 (m, 1H), 1.96-1.85 (m, 1H), 1.75-1.65 (m, 2H), 1.58-1.52 (m, 1H), 1.03 (d, J=6.2 Hz, 6H). LC-MS (ESI): m/z [M+H]⁺=482.2.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=10: 90 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for Hexane and B for EtOH (0.2% 2M NH₃ MeOH); Gradient: Mobile Phase A: Mobile Phase B=10: 90 (v/v); Flow Rate: 18 mL/min, Wave Length: UV 270 nm and 310 nm, Prep-HPLC Equipment: Prep-HPLC-Gilson, Back pressure: 100 bar; Column temperature: RT.

### Example 272: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl propylcarbamate

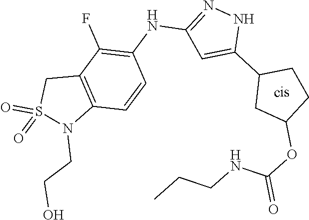 [SMILES: CCCNC(=O)OC1CCC(c2cc(Nc3ccc4c(c3F)CS(=O)(=O)N4CCO)n[nH]2)C1]

The titled compound was synthesized in the procedures similar to Example 271 in racemic form, which was further separated by Chiral Prep-HPLC to give:

Enantiomer 1 (Example 272a, 100% ee); Retention time: 4.55 min. ¹H NMR (500 MHz, DMSO-d₆) δ 11.69 (s, 1H), 8.00 (t, J=8.4 Hz, 1H), 7.89 (s, 1H), 7.04 (t, J=5.3 Hz, 1H), 6.76 (d, J=8.8 Hz, 1H), 5.67 (s, 1H), 5.12-4.88 (m, 2H), 4.69 (s, 2H), 3.66-3.60 (m, 2H), 3.53 (t, J=6.2 Hz, 2H), 3.09-2.99 (m, 1H), 2.91 (dd, J=13.2, 6.6 Hz, 2H), 2.45-2.37 (m, 1H), 2.07-1.97 (m, 1H), 1.97-1.86 (m, 1H), 1.79-1.66 (m, 2H), 1.63-1.53 (m, 1H), 1.42-1.34 (m, 2H), 0.82 (t, J=7.4 Hz, 3H). LC-MS (ESI): m/z [M+H]⁺=482.2.

Enantiomer 2 (Example 272b, 99.4% ee); Retention time: 5.29 min. ¹H NMR (500 MHz, DMSO-d₆) δ 11.69 (s, 1H), 8.00 (t, J=8.4 Hz, 1H), 7.89 (s, 1H), 7.04 (t, J=5.3 Hz, 1H), 6.76 (d, J=8.8 Hz, 1H), 5.67 (s, 1H), 5.12-4.88 (m, 2H), 4.69 (s, 2H), 3.66-3.60 (m, 2H), 3.53 (t, J=6.2 Hz, 2H), 3.09-2.99 (m, 1H), 2.91 (dd, J=13.2, 6.6 Hz, 2H), 2.45-2.37 (m, 1H), 2.07-1.97 (m, 1H), 1.97-1.86 (m, 1H), 1.79-1.66 (m, 2H), 1.63-1.53 (m, 1H), 1.42-1.34 (m, 2H), 0.82 (t, J=7.4 Hz, 3H). LC-MS (ESI): m/z [M+H]⁺=482.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=50: 50 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 273: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl((S)-sec-butyl)carbamate

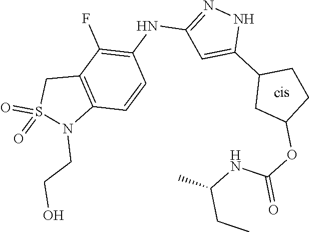 [SMILES: CC[C@H](C)NC(=O)OC1CCC(c2cc(Nc3ccc4c(c3F)CS(=O)(=O)N4CCO)n[nH]2)C1]

The titled compound was synthesized in the procedures similar to Example 271. ¹H NMR (500 MHz, DMSO-d₆) δ 11.72 (s, 1H), 7.87 (s, 2H), 6.87 (d, J=6.9 Hz, 1H), 6.70 (d, J=8.8 Hz, 1H), 5.61 (s, 1H), 4.92 (s, 1H), 4.63 (s, 2H), 3.58-3.55 (m, 2H), 3.49-3.44 (m, 3H), 3.02-2.90 (m, 1H), 2.39-2.32 (m, 1H), 1.98-1.90 (m, 1H), 1.88-1.77 (m, 1H), 1.70-1.62 (m, 2H), 1.55-1.49 (m, 1H), 1.39-1.26 (m, 2H), 1.04-0.96 (m, 3H), 0.86-0.76 (m, 3H). LC-MS (ESI): m/z [M+H]⁺=496.2.

### Example 274: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl(1-methylcyclopropyl)carbamate

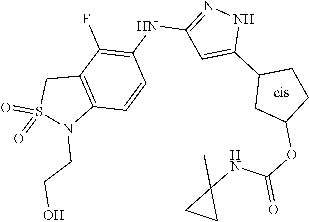 [SMILES: CC1(NC(=O)OC2CCC(c3cc(Nc4ccc5c(c4F)CS(=O)(=O)N5CCO)n[nH]3)C2)CC1]

The titled compound was synthesized in the procedures similar to Example 271. ¹H NMR (500 MHz, DMSO-d₆) δ 11.69 (s, 1H), 7.98 (s, 1H), 7.89 (s, 1H), 6.76 (d, J=8.8 Hz, 1H), 5.66 (s, 1H), 4.95 (t, J=5.5 Hz, 2H), 4.69 (s, 2H), 3.66-3.60 (m, 2H), 3.56-3.50 (m, 2H), 3.10-2.94 (m, 1H), 2.50-2.44 (m, 1H), 2.07-1.97 (m, 1H), 1.96-1.85 (m, 1H), 1.75-1.65 (m, 2H), 1.58-1.52 (m, 1H), 1.23 (s, 3H), 0.62-0.56 (m, 2H), 0.50-0.44 (m, 2H). LC-MS (ESI): m/z [M+H]⁺=494.2.

### Example 275: cis-3-(3-((1-cyclopropyl-4-fluoro-2,2-dioxido-1,3-dihydrobenzo[c]isothiazol-5-yl)amino)-1H-pyrazol-5-yl)cyclopentyl isopropylcarbamate

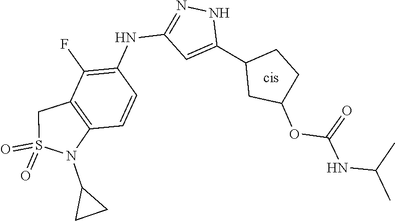 [SMILES: CC(C)NC(=O)OC1CCC(c2cc(Nc3ccc4c(c3F)CS(=O)(=O)N4C3CC3)n[nH]2)C1]

The titled compound was synthesized in the procedures similar to Example 203 in a racemic form, which was further separated by Chiral Prep-HPLC to give:

Enantiomer 1 (Example 275a, 100% ee); Retention time: 7.188 min. ¹H NMR (500 MHz, DMSO-d₆) δ=11.71 (s, 1H), 8.04 (s, 1H), 7.92 (s, 1H), 6.94 (d, J=7.4, 1H), 6.83 (d, J=8.7, 1H), 5.68 (s, 1H), 4.98 (s, 1H), 4.70 (s, 2H), 3.57 (m, 1H), 3.11-2.95 (m, 1H), 2.47-2.41 (m, 2H), 2.04-1.95 (m, 1H), 1.95-1.82 (m, 1H), 1.69 (m, 2H), 1.58 (m, 1H), 1.03 (m, 6H), 0.95-0.90 (m, 2H), 0.90-0.82 (m, 2H). LC-MS (ESI): m/z [M+H]⁺=478.32.

Enantiomer 2 (Example 275b,100% ee); Retention time: 9.131 min. ¹H NMR (500 MHz, DMSO-d₆) δ=11.71 (s, 1H), 8.05 (t, J=8.8, 1H), 7.93 (s, 1H), 6.94 (d, J=7.5, 1H), 6.83 (d, J=8.7, 1H), 5.68 (s, 1H), 4.99 (s, 1H), 4.70 (s, 2H), 3.57 (m, 1H), 3.11-2.93 (m, 1H), 2.48-2.40 (m, 2H), 2.05-1.96 (m, 1H), 1.96-1.83 (m, 1H), 1.69 (m, 2H), 1.58 (m, 1H), 1.03 (m, 6H), 0.98-0.91 (m, 2H), 0.91-0.84 (m, 2H). LC-MS (ESI): m/z [M+H]⁺=478.32.

Chiral analytical method: Column: CHIRALPAK IE 4.6×250 mm 5 μm; Mobile phase: A for Hex and B for EtOH (0.1% 2M NH₃ MeOH) (%); Gradient: Mobile Phase A: Mobile Phase B=10: 90 (v/v); HPLC Equipment: HPLC-Agilent; Column temperature: 35° C.

Chiral Prep-HPLC Condition: CHIRALPAK IE 21.2 mm×250 mm, 5 μm; Mobile phase: A for Hex and B for EtOH (0.1% 2M NH₃ MeOH); Gradient: Mobile Phase A: Mobile Phase B=10: 90 (v/v); Flow Rate: 18 mL/min, Wavelength: UV 210 nm and 270 nm, Prep-HPLC Equipment: Prep-HPLC-Gilson; Column temperature: 25° C.

### Example 276: cis-3-(3-((1-cyclopropyl-4-fluoro-2,2-dioxido-1,3-dihydrobenzo[c]isothiazol-5-yl)amino)-1H-pyrazol-5-yl)cyclopentyl propylcarbamate

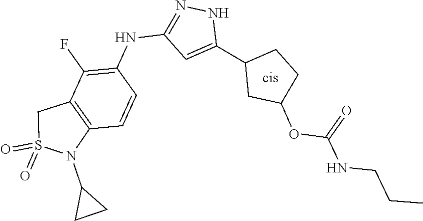 [SMILES: CCCNC(=O)OC1CCC(c2cc(Nc3ccc4c(c3F)CS(=O)(=O)N4C3CC3)n[nH]2)C1]

The titled compound was synthesized in the procedures similar to Example 203. ¹H NMR (500 MHz, DMSO-d₆) δ=11.71 (s, 1H), 8.05 (t, J=8.3, 1H), 7.92 (s, 1H), 7.04 (t, J=5.3, 1H), 6.83 (d, J=8.7, 1H), 5.68 (s, 1H), 4.99 (s, 1H), 4.70 (s, 2H), 3.09-2.96 (m, 1H), 2.91 (m, 2H), 2.48-2.40 (m, 2H), 2.00 (m, 1H), 1.96-1.81 (m, 1H), 1.79-1.63 (m, 2H), 1.60 (m, 1H), 1.38 (m, 2H), 0.96-0.91 (m, 2H), 0.91-0.85 (m, 2H), 0.82 (t, J=7.4, 3H). LC-MS (ESI): m/z [M+H]⁺=478.37.

### Example 277: cis-3-(3-((1-cyclopropyl-4-fluoro-2,2-dioxido-1,3-dihydrobenzo[c]isothiazol-5-yl)amino)-1H-pyrazol-5-yl)cyclopentyl(1-methylcyclopropyl)carbamate

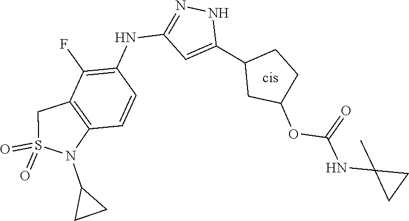 [SMILES: CC1(NC(=O)OC2CCC(c3cc(Nc4ccc5c(c4F)CS(=O)(=O)N5C4CC4)n[nH]3)C2)CC1]

The titled compound was synthesized in the procedures similar to Example 203. ¹H NMR (500 MHz, DMSO-d₆) δ=11.71 (s, 1H), 8.05 (t, J=8.7, 1H), 7.92 (s, 1H), 7.34 (s, 1H), 6.83 (d, J=8.7, 1H), 5.67 (s, 1H), 4.98 (s, 1H), 4.69 (s, 2H), 3.01 (m, 1H), 2.48-2.40 (m, 2H), 2.00 (m, 1H), 1.88 (m, 1H), 1.68 (m, 2H), 1.55 (m, 1H), 1.23 (s, 3H), 0.97-0.91 (m, 2H), 0.91-0.84 (m, 2H), 0.59 (m, 2H), 0.47 (m, 2H). LC-MS (ESI): m/z [M+H]⁺=490.37.

### Example 278: cis-3-(3-((1-cyclopropyl-4-fluoro-2,2-dioxido-1,3-dihydrobenzo[c]isothiazol-5-yl)amino)-1H-pyrazol-5-yl)cyclopentyl((S)-sec-butyl)carbamate

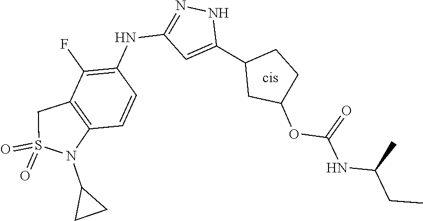 [SMILES: CC[C@H](C)NC(=O)OC1CCC(c2cc(Nc3ccc4c(c3F)CS(=O)(=O)N4C3CC3)n[nH]2)C1]

The titled compound was synthesized in the procedures similar to Example 203. ¹H NMR (500 MHz, DMSO-d₆) δ=11.71 (s, 1H), 8.05 (t, J=8.5, 1H), 7.92 (s, 1H), 6.88 (d, J=8.1, 1H), 6.83 (d, J=8.7, 1H), 5.68 (s, 1H), 4.98 (s, 1H), 4.70 (s, 2H), 3.37 (m, 1H), 3.03 (m, 1H), 2.47-2.41 (m, 2H), 2.01 (m, 1H), 1.90 (m, 1H), 1.70 (m, 2H), 1.58 (m, 1H), 1.36 (m, 2H), 1.00 (m, 3H), 0.94 (m, 2H), 0.88 (m, 2H), 0.80 (m, 3H). LC-MS (ESI): m/z [M+H]⁺=492.34.

### Example 279: cis-3-(3-((7-fluoro-2,2-dioxido-1,3-dihydrobenzo[c]isothiazol-5-yl)amino)-1H-pyrazol-5-yl)cyclopentyl isopropylcarbamate

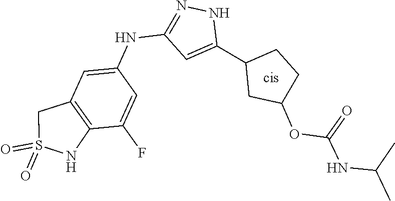 [SMILES: CC(C)NC(=O)OC1CCC(c2cc(Nc3cc(F)c4c(c3)CS(=O)(=O)N4)n[nH]2)C1]

The titled compound was synthesized in the procedures similar to Example 203. ¹H NMR (500 MHz, DMSO-d₆)) δ 11.78 (s, 1H), 9.96 (s, 1H), 8.52 (s, 1H), 7.31 (d, J=13.1 Hz, 1H), 7.07 (s, 1H), 6.95 (d, J=7.2 Hz, 1H), 5.60 (s, 1H), 5.00 (s, 1H), 4.53 (s, 2H), 3.62-3.55 (m, 1H), 3.10-2.98 (m, 1H), 2.46-2.39 (m, 1H), 2.03-1.92 (m, 1H), 1.97-1.86 (m, 1H), 1.75-1.59 (m, 2H), 1.63-1.53 (m, 1H), 1.03 (d, J=6.3 Hz, 6H). LC-MS (ESI): m/z [M+H]⁺=438.4.

### Example 280: cis-3-(3-((7-fluoro-1-methyl-2,2-dioxido-1,3-dihydrobenzo[c]isothiazol-5-yl)amino)-1H-pyrazol-5-yl)cyclopentyl isopropylcarbamate

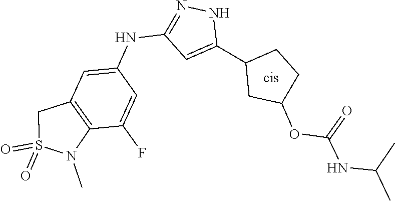 [SMILES: CC(C)NC(=O)OC1CCC(c2cc(Nc3cc(F)c4c(c3)CS(=O)(=O)N4C)n[nH]2)C1]

The titled compound was synthesized in the procedures similar to Example 259. ¹H NMR (500 MHz, DMSO-d₆) δ 11.81 (s, 1H), 8.64 (s, 1H), 7.35 (d, J=14.5 Hz, 1H), 7.09 (s, 1H), 6.96 (d, J=7.6 Hz, 1H), 5.60 (s, 1H), 4.99 (s, 1H), 4.66 (s, 2H), 3.62-3.55 (m, 1H), 3.11-3.00 (m, 4H), 2.46-2.37 (m, 1H), 2.03-1.92 (m, 1H), 1.97-1.86 (m, 1H), 1.75-1.59 (m, 2H), 1.63-1.53 (m, 1H), 1.03 (d, J=6.2 Hz, 6H). LC-MS (ESI): m/z [M+H]⁺=438.4.

### Example 281: cis-3-(3-((7-fluoro-2,2-dioxido-1-(2,2,2-trifluoroethyl)-1,3-dihydrobenzo[c]isothiazol-5-yl)amino)-1H-pyrazol-5-yl)cyclopentyl isopropylcarbamate

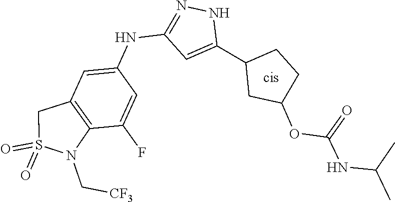 [SMILES: CC(C)NC(=O)OC1CCC(c2cc(Nc3cc(F)c4c(c3)CS(=O)(=O)N4CC(F)(F)F)n[nH]2)C1]

The titled compound was synthesized in the procedures similar to Example 259. ¹H NMR (500 MHz, DMSO-d₆) δ 11.82 (s, 1H), 8.72 (s, 1H), 7.39 (d, J=14.2 Hz, 1H), 7.10 (s, 1H), 6.94 (d, J=7.2 Hz, 1H), 5.61 (s, 1H), 4.99 (s, 1H), 4.73 (s, 2H), 4.30 (q, J=9.2 Hz, 2H), 3.64-3.52 (m, 1H), 3.09-3.00 (m, 1H), 2.46-2.38 (m, 1H), 2.03-1.92 (m, 1H), 1.97-1.86 (m, 1H), 1.75-1.59 (m, 2H), 1.63-1.53 (m, 1H), 1.03 (d, J=6.1 Hz, 6H). LC-MS (ESI): m/z [M+H]⁺=520.4.

### Example 282: cis-3-(3-((7-fluoro-1-(methoxymethyl)-2,2-dioxido-1,3-dihydrobenzo[c]isothiazol-5-yl)amino)-1H-pyrazol-5-yl)cyclopentyl isopropylcarbamate

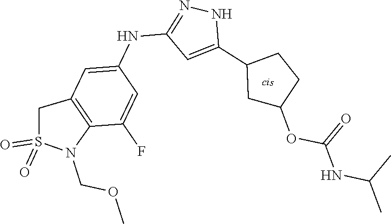 [SMILES: COCN1c2c(F)cc(Nc3cc(C4CCC(OC(=O)NC(C)C)C4)[nH]n3)cc2CS1(=O)=O]

The titled compound was synthesized in the procedures similar to Example 259. ¹H NMR (500 MHz, DMSO-d₆) δ 11.82 (s, 1H), 8.63 (s, 1H), 7.36 (d, J=13.9 Hz, 1H), 7.10 (s, 1H), 6.94 (d, J=7.4 Hz, 1H), 5.61 (s, 1H), 4.99 (s, 1H), 4.56 (s, 2H), 3.66 (t, J=5.7 Hz, 2H), 3.58 (dd, J=13.2, 6.4 Hz, 1H), 3.46 (t, J=5.7 Hz, 2H), 3.18 (s, 3H), 3.04 (dd, J=15.1, 7.1 Hz, 1H), 2.45 (dd, J=14.0, 7.2 Hz, 1H), 2.01 (dd, J=15.8, 7.6 Hz, 1H), 1.93-1.85 (m, 1H), 1.70 (t, J=12.2 Hz, 2H), 1.59 (s, 1H), 1.03 (d, J=6.3 Hz, 6H). LC-MS (ESI): m/z [M+H]⁺=482.4.

### Example 283: cis-3-(3-((7-fluoro-1-(oxetan-3-yl)-2,2-dioxido-1,3-dihydrobenzo[c]isothiazol-5-yl)amino)-1H-pyrazol-5-yl)cyclopentyl isopropylcarbamate

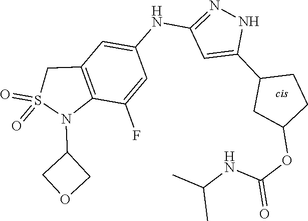 [SMILES: CC(C)NC(=O)OC1CCC(c2cc(Nc3cc(F)c4c(c3)CS(=O)(=O)N4C3COC3)n[nH]2)C1]

The titled compound was synthesized in the procedures similar to Example 63. ¹H NMR (500 MHz, DMSO-d₆) δ 11.83 (s, 1H), 8.76 (s, 1H), 7.35 (d, J=14.0 Hz, 1H), 7.13 (s, 1H), 6.94 (d, J=6.7 Hz, 1H), 5.62 (s, 1H), 5.05-4.95 (m, 1H), 4.86-4.78 (m, 3H), 4.72-4.64 (m, 4H), 3.65-3.52 (m, 1H), 3.10-2.98 (m, 1H), 2.48-2.42 (m, 1H), 2.05-1.96 (m, 1H), 1.96-1.84 (m, 1H), 1.78-1.66 (m, 2H), 1.65-1.54 (m, 1H), 1.03 (d, J=6.0 Hz, 6H). LC-MS (ESI): m/z [M+H]⁺=494.30.

### Example 284: cis-3-(3-((7-fluoro-1,3-bis(methyl-d3)-2,2-dioxido-1,3-dihydrobenzo[c]isothiazol-5-yl)amino)-1H-pyrazol-5-yl)cyclopentyl propylcarbamate

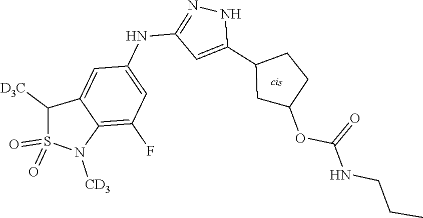 [SMILES: [2H]C([2H])([2H])C1c2cc(Nc3cc(C4CCC(OC(=O)NCCC)C4)[nH]n3)cc(F)c2N(C([2H])([2H])[2H])S1(=O)=O]

The titled compound was synthesized in the procedures similar to Example 253. ¹H NMR (500 MHz, DMSO-d₆) δ 11.81 (s, 1H), 8.61 (s, 1H), 7.35 (d, J=14.5 Hz, 1H), 7.05 (dd, J=16.1, 10.6 Hz, 2H), 5.60 (s, 1H), 4.99 (d, J=3.4 Hz, 1H), 4.65 (s, 1H), 3.36-3.25 (m, 1H), 3.10-2.99 (m, 1H), 3.09-2.95 (m, 1H), 2.50-2.44 (m, 1H), 2.06-1.99 (m, 1H), 1.95-1.85 (m, 1H), 1.78-1.65 (m, 2H), 1.64-1.55 (m, 1H), 1.47-1.29 (m, 2H), 0.82 (t, J=7.4 Hz, 3H). LC-MS (ESI): m/z [M+H]⁺=472.2.

### Example 285: cis-3-(3-((7-fluoro-1-(methyl-d3)-2,2-dioxido-1,3-dihydrobenzo[c]isothiazol-5-yl)amino)-1H-pyrazol-5-yl)cyclopentyl((S)-sec-butyl)carbamate

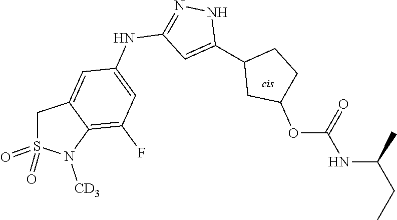 [SMILES: [2H]C([2H])([2H])N1c2c(F)cc(Nc3cc(C4CCC(OC(=O)N[C@@H](C)CC)C4)[nH]n3)cc2CS1(=O)=O]

The titled compound was synthesized in the procedures similar to Example 35. ¹H NMR (500 MHz, DMSO-d₆) δ 11.76 (s, 1H), 8.61 (s, 1H), 7.36 (d, J=14.6 Hz, 1H), 7.09 (s, 1H), 6.88 (d, J=8.2 Hz, 1H), 5.60 (s, 1H), 4.98 (s, 1H), 4.65 (s, 2H), 3.42-3.36 (m, 1H), 3.12-2.96 (m, 1H), 2.43-2.39 (m, 1H), 2.04-1.97 (m, 1H), 1.95-1.85 (m, 1H), 1.78-1.65 (m, 2H), 1.64-1.55 (m, 1H), 1.40-1.28 (m, 2H), 1.00 (d, J=6.4 Hz, 3H), 0.80 (t, J=6.3 Hz, 3H). LC-MS (ESI): m/z [M+H]⁺=469.2.

### Example 286: cis-3-(3-((7-fluoro-1,3-bis(methyl-d3)-2,2-dioxido-1,3-dihydrobenzo[c]isothiazol-5-yl)amino)-1H-pyrazol-5-yl)cyclopentyl((S)-sec-butyl)carbamate

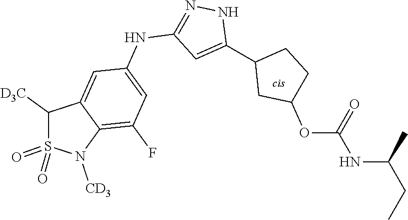 [SMILES: [2H]C([2H])([2H])C1c2cc(Nc3cc(C4CCC(OC(=O)N[C@@H](C)CC)C4)[nH]n3)cc(F)c2N(C([2H])([2H])[2H])S1(=O)=O]

The titled compound was synthesized in the procedures similar to Example 253. ¹H NMR (500 MHz, DMSO-d₆) δ 11.76 (s, 1H), 8.61 (s, 1H), 7.36 (d, J=14.6 Hz, 1H), 7.09 (s, 1H), 6.88 (d, J=8.2 Hz, 1H), 5.60 (s, 1H), 4.98 (s, 1H), 4.65 (s, 2H), 3.42-3.36 (m, 1H), 3.12-2.96 (m, 1H), 2.43-2.39 (m, 1H), 2.04-1.97 (m, 1H), 1.95-1.85 (m, 1H), 1.78-1.65 (m, 2H), 1.64-1.55 (m, 1H), 1.40-1.28 (m, 2H), 1.00 (d, J=6.4 Hz, 3H), 0.80 (t, J=6.3 Hz, 3H). LC-MS (ESI): m/z [M+H]⁺=486.2.

### Example 287: cis-3-(3-((7-fluoro-1,3-bis(methyl-d3)-2,2-dioxido-1,3-dihydrobenzo[c]isothiazol-5-yl)amino)-1H-pyrazol-5-yl)cyclopentyl(1-methylcyclopropyl)carbamate

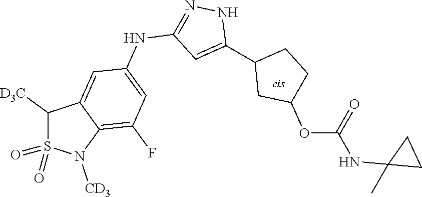 [SMILES: [2H]C([2H])([2H])C1c2cc(Nc3cc(C4CCC(OC(=O)NC5(C)CC5)C4)[nH]n3)cc(F)c2N(C([2H])([2H])[2H])S1(=O)=O]

The titled compound was synthesized in the procedures similar to Example 253. ¹H NMR (500 MHz, DMSO-d₆) δ 11.76 (s, 1H), 8.60 (s, 1H), 7.35 (d, J=10.7 Hz, 2H), 7.08 (s, 1H), 5.59 (s, 1H), 4.98 (s, 1H), 4.65 (s, 1H), 3.10-2.98 (m, 1H), 2.50-2.44 (m, 1H), 2.10-1.95 (m, 2H), 1.93-1.84 (m, 1H), 1.75-1.65 (m, 2H), 1.59-1.52 (m, 1H), 1.23 (s, 3H), 0.59 (s, 2H), 0.47 (d, J=6.0 Hz, 2H). LC-MS (ESI): m/z [M+H]⁺=484.3.

### Example 288: cis-3-(3-((1-cyclopropyl-7-fluoro-2,2-dioxido-1,3-dihydrobenzo[c]isothiazol-5-yl)amino)-1H-pyrazol-5-yl)cyclopentyl isopropylcarbamate

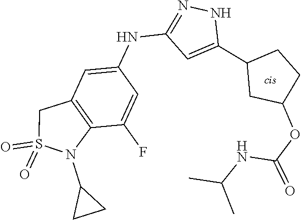 [SMILES: CC(C)NC(=O)OC1CCC(c2cc(Nc3cc(F)c4c(c3)CS(=O)(=O)N4C3CC3)n[nH]2)C1]

The titled compound was synthesized in the procedures similar to Example 259. ¹H NMR (500 MHz, DMSO-d₆) δ 11.77 (s, 1H), 8.61 (s, 1H), 7.35 (d, J=14.5 Hz, 1H), 7.08 (s, 1H), 6.94 (d, J=7.1 Hz, 1H), 5.60 (s, 1H), 4.98 (s, 1H), 4.64 (s, 2H), 3.62-3.53 (m, 1H), 3.08-2.98 (m, 1H), 2.66-2.61 (m, 1H), 2.48-2.43 (m, 1H), 2.05-1.85 (m, 2H), 1.78-1.54 (m, 3H), 1.03 (d, J=6.4 Hz, 6H), 0.94 (s, 2H), 0.86-0.80 (m, 2H). LC-MS (ESI): m/z [M+H]⁺=478.2.

### Example 289: cis-3-(3-((7-fluoro-1-(methyl-d3)-2,2-dioxido-1,3-dihydrobenzo[c]isothiazol-5-yl)amino-1H-pyrazol-5-yl)cyclopentyl isopropylcarbamate

### Step 1:5-bromo-7-fluoro-1,3-dihydrobenzo[c]isothiazole 2,2-dioxide

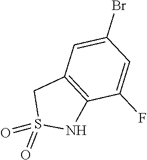 [SMILES: O=S1(=O)Cc2cc(Br)cc(F)c2N1]

The titled compound was synthesized in the procedures similar to Example 203, step 1 to step 4.

LC-MS (ESI): m/z [M+H]⁺=266.4.

### Step 2:5-bromo-7-fluoro-1-(methyl-d3)-1,3-dihydrobenzo[c]isothiazole 2,2-dioxide

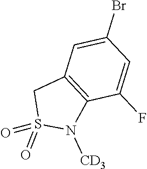 [SMILES: [2H]C([2H])([2H])N1c2c(F)cc(Br)cc2CS1(=O)=O]

To a stirring solution of 5-bromo-7-fluoro-1,3-dihydrobenzo[c]isothiazole 2,2-dioxide (750 mg, 2.83 mmol) in DMF (20 mL) was added K₂CO₃ (781 mg, 5.66 mmol), CD3I (820.7 mg, 5.66 mmol). The solution was stirred for 3 h at RT. The mixture was concentrated under reduced pressure. The residue was purified by silica gel column chromatography, eluting with PE/EtOAc (3:1) to afford the product (700 mg, 87.3%), [M+H]⁺=282.9.

### Step 3: cis-3-(3-((7-fluoro-1-(methyl-d3)-2,2-dioxido-1,3-dihydrobenzo[c]isothiazol-5-yl)amino)-1H-pyrazol-5-yl)cyclopentyl isopropylcarbamate

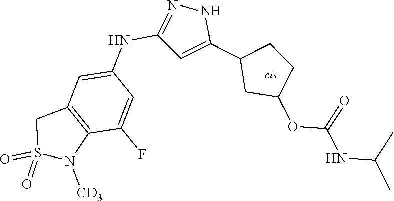 [SMILES: [2H]C([2H])([2H])N1c2c(F)cc(Nc3cc(C4CCC(OC(=O)NC(C)C)C4)[nH]n3)cc2CS1(=O)=O]

A mixture of 5-bromo-7-fluoro-1-(methyl-d3)-1,3-dihydrobenzo[c]isothiazole 2,2-dioxide (150 mg, 0.57 mmol), cis-3-(3-amino-1H-pyrazol-5-yl)cyclopentyl isopropylcarbamate (144.2 mg, 0.57 mmol), Brettphos Pd G3 (51.3 mg, 0.057 mmol), K₂CO₃ (235.9 mg, 1.71 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product 110 mg in racemic form, which was further separated by Chiral Prep-HPLC to give:

Enantiomer 1 (Example 289a, 100% ee); Retention time: 4.14 min. ¹H NMR (500 MHz, DMSO-d₆) δ 11.76 (s, 1H), 8.61 (s, 1H), 7.36 (d, J=14.4 Hz, 1H), 7.09 (s, 1H), 6.94 (d, J=7.3 Hz, 1H), 5.60 (s, 1H), 4.98 (s, 1H), 4.65 (s, 2H), 3.62-3.56 (m, 1H), 3.09-2.95 (m, 1H), 2.50-2.44 (m, 1H), 2.06-1.99 (m, 1H), 1.96-1.86 (m, 1H), 1.78-1.66 (m, 2H), 1.65-1.57 (m, 1H), 1.04 (t, J=7.8 Hz, 6H). LC-MS (ESI): m/z [M+H]⁺=455.2.

Enantiomer 2 (Example 289b, 100% ee); Retention time: 5.12 min. ¹H NMR (500 MHz, DMSO-d₆) δ 11.76 (s, 1H), 8.61 (s, 1H), 7.36 (d, J=14.4 Hz, 1H), 7.09 (s, 1H), 6.94 (d, J=7.3 Hz, 1H), 5.60 (s, 1H), 4.98 (s, 1H), 4.65 (s, 2H), 3.62-3.56 (m, 1H), 3.09-2.95 (m, 1H), 2.50-2.44 (m, 1H), 2.06-1.99 (m, 1H), 1.96-1.86 (m, 1H), 1.78-1.66 (m, 2H), 1.65-1.57 (m, 1H), 1.04 (t, J=7.8 Hz, 6H). LC-MS (ESI): m/z [M+H]⁺=455.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=50: 50 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 290: cis-3-(3-((7-fluoro-1-(methyl-d3)-2,2-dioxido-1,3-dihydrobenzo[c]isothiazol-5-yl)amino)-1H-pyrazol-5-yl)cyclopentyl propylcarbamate

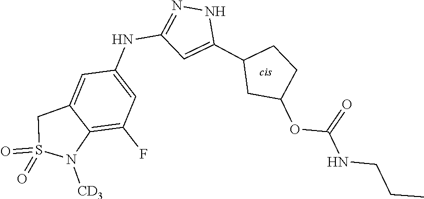 [SMILES: [2H]C([2H])([2H])N1c2c(F)cc(Nc3cc(C4CCC(OC(=O)NCCC)C4)[nH]n3)cc2CS1(=O)=O]

The titled compound was synthesized in the procedures similar to Example 259 in racemic form, which was further purified with chiral-Prep HPLC to give:

Enantiomer 1 (Example 289a, 100% ee); Retention time: 5.349 min. ¹H NMR (500 MHz, DMSO-d₆) δ 11.81 (s, 1H), 8.61 (s, 1H), 7.35 (d, J=14.5 Hz, 1H), 7.05 (dd, J=16.1, 10.6 Hz, 2H), 5.60 (s, 1H), 4.99 (d, J=3.4 Hz, 1H), 4.65 (s, 2H), 3.36-3.25 (m, 1H), 3.10-2.99 (m, 1H), 3.09-2.95 (m, 1H), 2.50-2.44 (m, 1H), 2.06-1.99 (m, 1H), 1.95-1.85 (m, 1H), 1.78-1.65 (m, 2H), 1.64-1.55 (m, 1H), 1.47-1.29 (m, 2H), 0.82 (t, J=7.4 Hz, 3H). LC-MS (ESI): m/z [M+H]⁺=455.2.

Enantiomer 2 (Example 289a, 100% ee); Retention time: 7.027 min. ¹H NMR (500 MHz, DMSO-d₆) δ 11.81 (s, 1H), 8.61 (s, 1H), 7.35 (d, J=14.5 Hz, 1H), 7.05 (dd, J=16.1, 10.6 Hz, 2H), 5.60 (s, 1H), 4.99 (d, J=3.4 Hz, 1H), 4.65 (s, 2H), 3.36-3.25 (m, 1H), 3.10-2.99 (m, 1H), 3.09-2.95 (m, 1H), 2.50-2.44 (m, 1H), 2.06-1.99 (m, 1H), 1.95-1.85 (m, 1H), 1.78-1.65 (m, 2H), 1.64-1.55 (m, 1H), 1.47-1.29 (m, 2H), 0.82 (t, J=7.4 Hz, 3H). LC-MS (ESI): m/z [M+H]⁺=455.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=70: 30 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=50: 50 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 291: cis-3-(3-((7-fluoro-1-(methyl-d3)-2,2-dioxido-1,3-dihydrobenzo[c]isothiazol-5-yl)amino)-1H-pyrazol-5-yl)cyclopentyl(1-methylcyclopropyl)carbamate

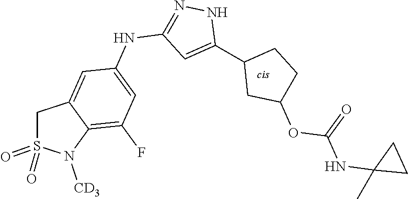 [SMILES: [2H]C([2H])([2H])N1c2c(F)cc(Nc3cc(C4CCC(OC(=O)NC5(C)CC5)C4)[nH]n3)cc2CS1(=O)=O]

The titled compound was synthesized in the procedures similar to Example 259 in racemic form, which was further purified with chiral Prep-HPLC to give:

Enantiomer 1 (Example 291a, 100% ee); Retention time: 5.797 min. ¹H NMR (500 MHz, DMSO-d₆) δ 11.76 (s, 1H), 8.60 (s, 1H), 7.35 (d, J=10.7 Hz, 2H), 7.08 (s, 1H), 5.59 (s, 1H), 4.98 (s, 1H), 4.65 (s, 2H), 3.10-2.98 (m, 1H), 2.50-2.44 (m, 1H), 2.10-1.95 (m, 2H), 1.93-1.84 (m, 1H), 1.75-1.65 (m, 2H), 1.59-1.52 (m, 1H), 1.23 (s, 3H), 0.59 (s, 2H), 0.47 (d, J=6.0 Hz, 2H). LC-MS (ESI): m/z [M+H]⁺=467.2. Enantiomer 2 (Example 291b, 100% ee); Retention time: 7.719 min. ¹H NMR (500 MHz, DMSO-d₆) δ 11.76 (s, 1H), 8.60 (s, 1H), 7.35 (d, J=10.7 Hz, 2H), 7.08 (s, 1H), 5.59 (s, 1H), 4.98 (s, 1H), 4.65 (s, 2H), 3.10-2.98 (m, 1H), 2.50-2.44 (m, 1H), 2.10-1.95 (m, 2H), 1.93-1.84 (m, 1H), 1.75-1.65 (m, 2H), 1.59-1.52 (m, 1H), 1.23 (s, 3H), 0.59 (s, 2H), 0.47 (d, J=6.0 Hz, 2H). LC-MS (ESI): m/z [M+H]⁺=467.2.

Chiral analytical method: Column: CHIRALPAK IE 4.6 mm×250 mm, 5 μm; Mobile phase: A for Hex and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for hexane and B for EtOH (0.2% 2M NH₃ MeOH); Gradient: Mobile Phase A: Mobile Phase B=50: 50 (v/v); Flow Rate: 18 mL/min, Wave Length: UV 280 nm and 300 nm, Prep-HPLC Equipment: Prep-HPLC-Gilson, Back pressure: 100 bar; Column temperature: RT.

### Example 292: cis-3-(3-((6-fluoro-2,2-dioxido-1,3-dihydrobenzo[c]isothiazol-5-yl)amino)-1H-pyrazol-5-yl)cyclopentyl isopropylcarbamate

### Step 1: S-(2-chloro-4-fluorobenzyl)ethanethioate

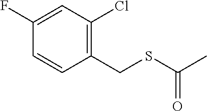

A mixture of 1-(bromomethyl)-2-chloro-4-fluorobenzene (25 g, 112.1 mmol) and potassium thioacetate (19.2 g, 168.2 mmol) in DMF (150 mL) was stirred for 16 h at 20° C. The resulting mixture was concentrated under reduced pressure. The residue was purified by silica gel column chromatography, eluting with PE/EtOAc (10:1) to afford the product (23 g, 93%), LC-MS (ESI): m/z [M+H]⁺=219.2.

### Step 2: (2-chloro-4-fluorophenyl) methanesulfonyl chloride

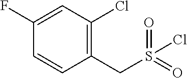 [SMILES: O=S(=O)(Cl)Cc1ccc(F)cc1Cl]

To a solution of HCl (2M, 30 mL) in acetonitrile (150 mL) was added NCS (56.2 g, 420 mmol) in portions under 10° C. Then a solution of S-(2-chloro-4-fluorobenzyl)ethanethioate (23 g, 105 mmol) in acetonitrile (30 mL) was added dropwise slowly to the reaction mixture under 20° C. The resulting mixture was stirred for 0.5 h at 20° C. The mixture was extracted with EtOAc (100 mL×2). The organic phase was washed with brine, dried over anhydrous sodium sulfate, concentrated under reduced pressure to afford the crude product (30 g) directly in the next step, LC-MS (ESI): m/z [M+H]⁺=243.2.

### Step 3: (2-chloro-4-fluorophenyl) methanesulfonamide

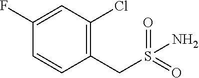 [SMILES: NS(=O)(=O)Cc1ccc(F)cc1Cl]

To a stirred solution of (2-chloro-4-fluorophenyl) methanesulfonyl chloride (30 g, 0.12 mol) in THF (150 mL) was added NH₃·H₂O (25%, 20 mL). The mixture was stirred for 16 h at 20° C. The mixture was concentrated under reduced pressure. The residue was purified by silica gel column chromatography, eluting with PE/EtOAc (1:1) to afford the product (9.8 g, 42% in two steps), LC-MS (ESI): m/z [M+H]⁺=224.2.

### Step 4:6-fluoro-1,3-dihydrobenzo[c]isothiazole 2,2-dioxide

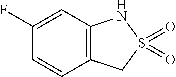 [SMILES: O=S1(=O)Cc2ccc(F)cc2N1]

A mixture of (2-chloro-4-fluorophenyl) methanesulfonamide (9.8 g, 43.7 mmol), Pd₂(dba)₃ (2 g, 2.2 mmol), 564483-19-8 (1.85 g, 4.37 mmol) and K₂CO₃ (12 g, 87.4 mmol) in THF (150 mL) was stirred at 80° C. for 16 h in the sealed tube under N₂ atmosphere. The mixture was cooled down to room temperature, then saturated NH₄Cl (aq) (15 mL) was added. The residue was concentrated under reduced pressure and purified by silica gel column chromatography, eluting with PE/EtOAc (1:1) to afford the product (5.6 g, 68%), LC-MS (ESI): m/z [M+H]⁺=188.2.

### Step 5:5-bromo-6-fluoro-1,3-dihydrobenzo[c]isothiazole 2,2-dioxide

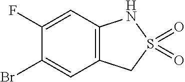 [SMILES: O=S1(=O)Cc2cc(Br)c(F)cc2N1]

To a stirred solution of 6-fluoro-1,3-dihydrobenzo[c]isothiazole 2,2-dioxide (5.6 g, 29.7 mmol) in AcOH (50 mL) was added Br₂ (3.8 g, 23.7 mmol) in AcOH (10 mL) dropwise at 0° C. The solution was stirred for 2 h at 20° C. The solution was concentrated under reduced pressure. The residue was purified by silica gel column chromatography, eluting with PE/EtOAc (1:1) to afford the product (4.5 g, 57%), LC-MS (ESI): m/z [M+H]⁺=266.3.

### Step 6:5-bromo-6-fluoro-1-(4-methoxybenzyl)-1,3-dihydrobenzo[c]isothiazole 2,2-dioxide

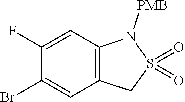

A mixture of 5-bromo-6-fluoro-1,3-dihydrobenzo[c]isothiazole 2,2-dioxide (1 g, 3.76 mmol), 4-Methoxybenzyl bromide (1.2 g, 6 mmol) and K₂CO₃ (1.5 g, 11 mmol) in DMF (20 mL) was stirred at 20° C. for 3 h. The solution was concentrated under reduced pressure. The residue was purified by silica gel column chromatography, eluting with PE/EtOAc (3:1) to afford the product (0.5 g, 34%), LC-MS (ESI): m/z [M+H]⁺=386.2.

### Step 7: cis-3-(3-((6-fluoro-1-(4-methoxybenzyl)-2,2-dioxido-1,3-dihydrobenzo[c]isothiazol-5-yl)amino)-1H-pyrazol-5-yl)cyclopentyl isopropylcarbamate

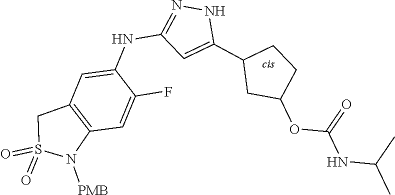

A mixture of 5-bromo-6-fluoro-1-(4-methoxybenzyl)-1,3-dihydrobenzo[c]isothiazole 2,2-dioxide (100 mg, 0.26 mmol), cis-3-(3-amino-1H-pyrazol-5-yl)cyclopentyl isopropylcarbamate (65 mg, 0.26 mmol), Brettephos Pd G3 (27 mg, 0.03 mmol) and K₂CO₃ (108 mg, 0.78 mmol) in t-BuOH (15 mL) was stirred for 16 h at 110° C. in the sealed tube under N₂ atmosphere. The solution was concentrated under reduced pressure. The residue was purified by silica gel column chromatography, eluting with EA/PE (10% to 100%) to afford the crude product (80 mg, 53%), LC-MS (ESI): m/z [M+H]⁺=558.3.

### Step 8: cis-3-(3-((6-fluoro-2,2-dioxido-1,3-dihydrobenzo[c]isothiazol-5-yl)amino)-1H-pyrazol-5-yl)cyclopentyl isopropylcarbamate

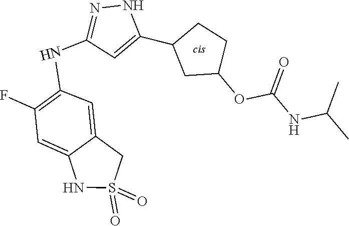 [SMILES: CC(C)NC(=O)OC1CCC(c2cc(Nc3cc4c(cc3F)NS(=O)(=O)C4)n[nH]2)C1]

To a stirred solution of cis-3-(3-((6-fluoro-1-(4-methoxybenzyl)-2,2-dioxido-1,3-dihydrobenzo[c]isothiazol-5-yl)amino)-1H-pyrazol-5-yl)cyclopentyl isopropylcarbamate (70 mg, 0.125 mmol) in DCM (5 mL) was added TFA (3 mL). The solution was stirred at 50° C. for 48 h. The solution was concentrated under reduced pressure. The residue was purified by prep HPLC (Waters SunFire C18: RD-CO-058 column, eluting with a gradient of acetonitrile/water containing 0.1% FA, 20%-55%) to afford the product (30 mg, 55%) in racemic form, which was further separated by Chiral Prep-HPLC to give:

Enantiomer 1 (Example 292a, 100% ee); Retention time: 5.94 min. ¹H NMR (500 MHz, DMSO-d₆) δ 11.97 (s, 1H), 8.73 (s, 1H), 8.15 (d, J=7.0 Hz, 1H), 7.77-7.54 (m, 2H), 6.94 (d, J=7.1 Hz, 1H), 5.85 (s, 1H), 5.08-4.93 (m, 1H), 4.30 (d, J=4.9 Hz, 2H), 3.65-3.52 (m, 1H), 3.13-3.01 (m, 1H), 2.48-2.42 (m, 1H), 2.06-1.98 (m, 1H), 1.96-1.86 (m, 1H), 1.78-1.66 (m, 2H), 1.65-1.53 (m, 1H), 1.03 (d, J=6.3 Hz, 6H). LC-MS (ESI): m/z [M+H]⁺=438.2.

Enantiomer 2 (Example 292b, 100% ee); Retention time: 7.28 min. ¹H NMR (500 MHz, DMSO-d₆) δ 11.97 (s, 1H), 8.73 (s, 1H), 8.15 (d, J=7.2 Hz, 1H), 7.76-7.54 (m, 2H), 6.94 (d, J=7.3 Hz, 1H), 5.84 (s, 1H), 5.11-4.93 (m, 1H), 4.30 (d, J=4.9 Hz, 2H), 3.68-3.51 (m, 1H), 3.15-2.97 (m, 1H), 2.46-2.42 (m, 1H), 2.08-1.96 (m, 1H), 1.95-1.85 (m, 1H), 1.79-1.66 (m, 2H), 1.65-1.53 (m, 1H), 1.03 (d, J=6.1 Hz, 6H). LC-MS (ESI): m/z [M+H]⁺=438.2.

Chiral analytical method: Column: CHIRALPAK IF 4.6 mm×150 mm, 5 μm; Mobile phase: A for Hexane and B for EtOH (0.1% 2M NH₃ MeOH); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IF 20 mm×250 mm, 5 μm; Mobile phase: A for Hexane and B for EtOH (0.2% 2M NH₃ MeOH); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 293: cis-3-(3-((6-fluoro-2,2-dioxido-1,3-dihydrobenzo[c]isothiazol-5-yl)amino)-1H-pyrazol-5-yl)cyclopentyl propylcarbamate

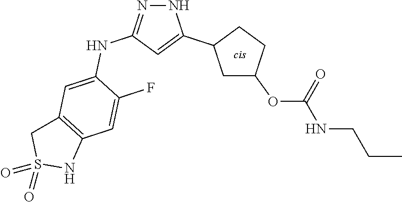 [SMILES: CCCNC(=O)OC1CCC(c2cc(Nc3cc4c(cc3F)NS(=O)(=O)C4)n[nH]2)C1]

The titled compound was synthesized in the procedures similar to Example 292. ¹H NMR (500 MHz, DMSO-d₆) δ 11.76 (brs, 1H), 10.06 (s, 1H), 8.07-7.73 (m, 2H), 7.04 (t, J=5.3 Hz, 1H), 6.72 (d, J=11.5 Hz, 1H), 5.69 (s, 1H), 5.08-4.85 (m, 1H), 4.46 (s, 2H), 3.11-2.97 (m, 1H), 2.97-2.80 (m, 2H), 2.48-2.40 (m, 1H), 2.06-1.95 (m, 1H), 1.95-1.83 (m, 1H), 1.80-1.64 (m, 2H), 1.62-1.52 (m, 1H), 1.45-1.30 (m, 2H), 0.82 (t, J=7.4 Hz, 3H). LC-MS (ESI): m/z [M+H]⁺=438.30.

### Example 294: cis-3-(3-((6-fluoro-2,2-dioxido-1,3-dihydrobenzo[c]isothiazol-5-yl)amino)-1H-pyrazol-5-yl)cyclopentyl((S)-sec-butyl)carbamate

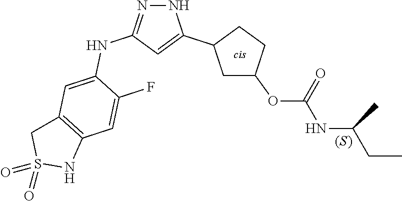

The titled compound was synthesized in the procedures similar to Example 292. ¹H NMR (500 MHz, DMSO-d₆) δ 11.73 (brs, 1H), 10.04 (s, 1H), 8.01 (d, J=6.9 Hz, 1H), 7.88 (s, 1H), 6.88 (d, J=7.9 Hz, 1H), 6.72 (d, J=11.4 Hz, 1H), 5.69 (s, 1H), 5.10-4.91 (m, 1H), 4.46 (s, 2H), 3.44-3.34 (m, 1H), 3.09-2.98 (m, 1H), 2.48-2.41 (m, 1H), 2.06-1.96 (m, 1H), 1.95-1.84 (m, 1H), 1.80-1.67 (m, 2H), 1.64-1.52 (m, 1H), 1.43-1.27 (m, 2H), 1.00 (d, J=5.3 Hz, 3H), 0.89-0.75 (m, 3H). LC-MS (ESI): m/z [M+H]⁺=452.30.

### Example 295: cis-3-(3-((6-fluoro-2,2-dioxido-1,3-dihydrobenzo[c]isothiazol-5-yl)amino)-1H-pyrazol-5-yl)cyclopentyl(1-methylcyclopropyl)carbamate

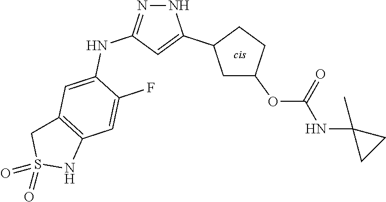 [SMILES: CC1(NC(=O)OC2CCC(c3cc(Nc4cc5c(cc4F)NS(=O)(=O)C5)n[nH]3)C2)CC1]

The titled compound was synthesized in the procedures similar to Example 292. ¹H NMR (500 MHz, DMSO-d₆) δ 11.67 (s, 1H), 10.03 (s, 1H), 8.04 (s, 1H), 7.86 (s, 1H), 7.34 (s, 1H), 6.71 (d, J=11.5 Hz, 1H), 5.68 (s, 1H), 5.10-4.89 (m, 1H), 4.46 (s, 2H), 3.10-2.95 (m, 1H), 2.48-2.40 (m, 1H), 2.05-1.96 (m, 1H), 1.95-1.83 (m, 1H), 1.79-1.61 (m, 2H), 1.60-1.47 (m, 1H), 1.23 (s, 3H), 0.65-0.55 (m, 2H), 0.52-0.42 (m, 2H). LC-MS (ESI): m/z [M+H]⁺=450.36.

### Example 296: cis-3-(3-((6-fluoro-1-(2-hydroxyethyl)-2,2-dioxido-1,3-dihydrobenzo[c]isothiazol-5-yl)amino)-1H-pyrazol-5-yl)cyclopentyl isopropylcarbamate

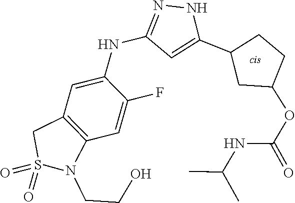 [SMILES: CC(C)NC(=O)OC1CCC(c2cc(Nc3cc4c(cc3F)N(CCO)S(=O)(=O)C4)n[nH]2)C1]

The titled compound was synthesized in the procedures similar to Example 271 in racemic form, which was further purified by Chiral-HPLC to yield:

Enantiomer 1 (Example 296a, 100% ee); Retention time: 5.991 min. ¹H NMR (500 MHz, DMSO-d₆) δ 11.67 (s, 1H), 8.07 (s, 1H), 7.87 (s, 1H), 6.99 (d, J=10 Hz, 1H), 6.94 (d, J=10 Hz, 1H), 5.68 (s, 1H), 5.03-4.91 (m, 2H), 4.57 (s, 2H), 3.66-3.54 (m, 3H), 3.54-3.49 (m, 2H), 3.08-2.97 (m, 1H), 2.47-2.40 (m, 1H), 2.06-1.95 (m, 1H), 1.94-1.85 (m, 1H), 1.77-1.64 (m, 2H), 1.62-1.53 (m, 1H), 1.09-0.97 (m, 6H). LC-MS (ESI): m/z [M+H]⁺=482.2.

Enantiomer 2 (Example 296b, 100% ee); Retention time: 7.053 min. ¹H NMR (500 MHz, DMSO-d₆) δ 11.67 (s, 1H), 8.08 (s, 1H), 7.87 (s, 1H), 6.99 (d, J=10 Hz, 1H), 6.94 (d, J=10 Hz, 1H), 5.68 (s, 1H), 5.02-4.91 (m, 2H), 4.56 (m, 2H), 3.66-3.55 (m, 3H), 3.54-3.48 (m, 2H), 3.07-2.96 (m, 1H), 2.47-2.43 (m, 1H), 2.04-1.95 (m, 1H), 1.94-1.83 (m, 1H), 1.76-1.62 (m, 2H), 1.61-1.50 (m, 1H), 1.07-0.96 (m, 6H). LC-MS (ESI): m/z [M+H]⁺=482.2.

Chiral analytical method: Column: CHIRALPAK IF 4.6 mm×150 mm, 5 μm; Mobile phase: A for Hexane and B for EtOH (0.1% 2M NH₃ MeOH); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IF 20 mm×250 mm, 5 μm; Mobile phase: A for Hexane and B for EtOH (0.2% 2M NH₃ MeOH); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 297: cis-3-(3-((6-fluoro-1-(2-hydroxyethyl)-2,2-dioxido-1,3-dihydrobenzo[c]isothiazol-5-yl)amino)-1H-pyrazol-5-yl)cyclopentyl propylcarbamate

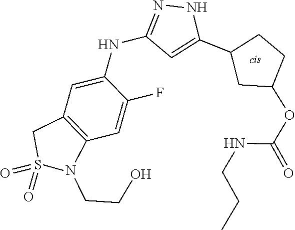 [SMILES: CCCNC(=O)OC1CCC(c2cc(Nc3cc4c(cc3F)N(CCO)S(=O)(=O)C4)n[nH]2)C1]

The titled compound was synthesized in the procedures similar to Example 217. ¹H NMR (500 MHz, DMSO-d₆) δ 11.67 (s, 1H), 8.07 (s, 1H), 7.87 (s 1H), 7.07-6.96 (m, 2H), 5.68 (s, 1H), 5.02-4.90 (m, 2H), 4.56 (s, 2H), 3.66-3.58 (m, 2H), 3.55-3.48 (m, 2H), 3.07-2.97 (m, 1H), 2.95-2.85 (m, 2H), 2.45-2.36 (m, 1H), 2.04-1.95 (m, 1H), 1.93-1.84 (m, 1H), 1.76-1.54 (m, 3H), 1.43-1.33 (m, 2H), 0.81 (d, J=5 Hz, 3H). LC-MS (ESI): m/z [M+H]⁺=482.2.

### Example 298: cis-3-(3-((6-fluoro-1-(2-hydroxyethyl)-2,2-dioxido-1,3-dihydrobenzo[c]isothiazol-5-yl)amino)-1H-pyrazol-5-yl)cyclopentyl isobutylcarbamate

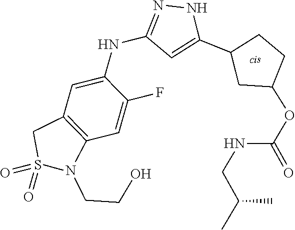

The titled compound was synthesized in the procedures similar to Example 271. ¹H NMR (500 MHz, DMSO-d₆) δ 11.67 (s, 1H), 8.07 (d, J=5 Hz, 1H), 7.87 (s, 1H), 6.99 (d, J=10 Hz, 1H), 6.88 (d, J=10 Hz, 1H), 5.68 (s, 1H), 5.05-4.91 (m, 2H), 4.56 (s, 2H), 3.67-3.59 (m, 2H), 3.57-3.48 (m, 2H), 3.42-3.34 (m, 1H), 3.07-2.97 (m, 1H), 2.47-2.41 (m, 1H), 2.05-1.96 (m, 1H), 1.95-1.85 (m, 1H), 1.78-1.63 (m, 2H), 1.62-1.53 (m, 1H), 1.41-1.29 (m, 2H), 1.03-0.96 (m, 3H), 0.84-0.76 (m, 3H). LC-MS (ESI): m/z [M+H]⁺=496.2.

### Example 299: cis-3-(3-((6-fluoro-1-(2-hydroxyethyl)-2,2-dioxido-1,3-dihydrobenzo[c]isothiazol-5-yl)amino)-1H-pyrazol-5-yl)cyclopentyl(1-methylcyclopropyl)carbamate

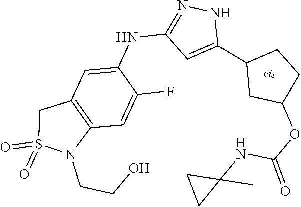 [SMILES: CC1(NC(=O)OC2CCC(c3cc(Nc4cc5c(cc4F)N(CCO)S(=O)(=O)C5)n[nH]3)C2)CC1]

The titled compound was synthesized in the procedures similar to Example 271. ¹H NMR (500 MHz, DMSO-d₆) δ 11.66 (s, 1H), 8.10-8.00 (m, 1H), 7.87 (s, 1H), 7.34 (s, 1H), 6.99 (d, J=10 Hz, 1H), 5.67 (s, 1H), 5.04-4.91 (m, 2H), 4.56 (s, 2H), 3.67-3.59 (m, 2H), 3.56-3.48 (m, 2H), 3.09-2.97 (m, 1H), 2.45-2.40 (m, 1H), 2.05-1.94 (m, 1H), 1.93-1.83 (m, 1H), 1.78-1.62 (m, 2H), 1.60-1.50 (m, 1H), 1.23 (s, 3H), 0.62-0.55 (m, 2H), 0.50-0.43 (m, 2H). LC-MS (ESI): m/z [M+H]⁺=494.2.

### Example 300: cis-3-(3-((6-fluoro-1-methyl-2,2-dioxido-1,3-dihydrobenzo[c]isothiazol-5-yl)amino)-1H-pyrazol-5-yl)cyclopentyl isopropylcarbamate

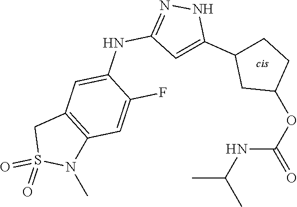 [SMILES: CC(C)NC(=O)OC1CCC(c2cc(Nc3cc4c(cc3F)N(C)S(=O)(=O)C4)n[nH]2)C1]

The titled compound was synthesized in the procedures similar to Example 35. ¹H NMR (500 MHz, DMSO-d₆) δ 11.68 (s, 1H), 8.09 (s, 1H), 7.88 (s, 1H), 6.94 (d, J=11.8 Hz, 2H), 5.68 (s, 1H), 4.99 (s, 1H), 4.59 (s, 2H), 3.65-3.50 (m, 1H), 3.08-3.00 (m, 1H), 2.97 (s, 3H), 2.46-2.40 (m, 1H), 2.05-1.98 (m, 1H), 1.95-1.84 (m, 1H), 1.70-1.68 (m, 2H), 1.57-1.45 (m, 1H), 1.03 (d, J=6.4 Hz, 6H). LC-MS (ESI): m/z [M+H]⁺=452.2.

### Example 301: cis-3-(3-((6-fluoro-1-(oxetan-3-yl)-2,2-dioxido-1,3-dihydrobenzo[c]isothiazol-5-yl)amino)-1H-pyrazol-5-yl)cyclopentyl isopropylcarbamate

### Step 1:5-bromo-6-fluoro-1-(oxetan-3-yl)-1,3-dihydrobenzo[c]isothiazole 2,2-dioxide

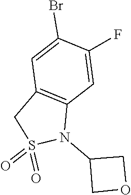 [SMILES: O=S1(=O)Cc2cc(Br)c(F)cc2N1C1COC1]

A solution of 5-bromo-6-fluoro-1,3-dihydrobenzo[c]isothiazole 2,2-dioxide (0.3 g, 1.13 mmol), oxetan-3-ol (167 mg, 2.26 mmol) and CMBP (817 mg, 3.39 mmol) in toluene (15 mL) was stirred for 16 h at 95° C. under N₂ atmosphere. The solution was concentrated under reduced pressure. The residue was purified by silica gel column chromatography, eluting with PE/EtOAc (2:1) to afford the product (150 mg, 41%), LC-MS (ESI): m/z [M+H]⁺=322.2.

### Step 2: cis-3-(3-((6-fluoro-1-(oxetan-3-yl)-2,2-dioxido-1,3-dihydrobenzo[c]isothiazol-5-yl)amino)-1H-pyrazol-5-yl)cyclopentyl isopropylcarbamate

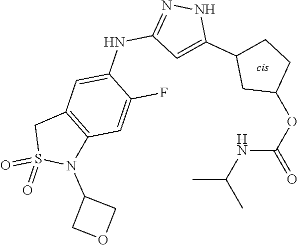 [SMILES: CC(C)NC(=O)OC1CCC(c2cc(Nc3cc4c(cc3F)N(C3COC3)S(=O)(=O)C4)n[nH]2)C1]

The titled compound was synthesized in the procedures similar to Example 34. ¹H NMR (500 MHz, DMSO-d₆) δ 11.74 (s, 1H), 8.2-8.08 (m, 1H), 8.02 (d, J=1.8 Hz, 1H), 6.94 (d, J=7.7 Hz, 1H), 6.88 (d, J=11.9 Hz, 1H), 5.70 (s, 1H), 5.02-4.95 (m, 3H), 4.91 (t, J=6.7 Hz, 2H), 4.89-4.82 (m, 1H), 4.68 (s, 2H), 3.65-3.50 (m, 1H), 3.09-2.99 (m, 1H), 2.48-2.41 (m, 1H), 2.06-1.96 (m, 1H), 1.96-1.85 (m, 1H), 1.77-1.65 (m, 2H), 1.64-1.52 (m, 1H), 1.03 (d, J=6.4 Hz, 6H). LC-MS (ESI): m/z [M+H]⁺=494.30.

### Example 302: cis-3-(3-((1-cyclopropyl-6-fluoro-2,2-dioxido-1,3-dihydrobenzo[c]isothiazol-5-yl)amino)-1H-pyrazol-5-yl)cyclopentyl isopropylcarbamate

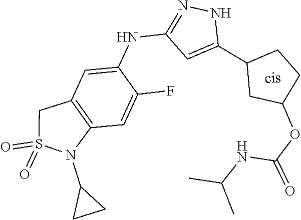 [SMILES: CC(C)NC(=O)OC1CCC(c2cc(Nc3cc4c(cc3F)N(C3CC3)S(=O)(=O)C4)n[nH]2)C1]

The titled compound was synthesized in the procedures similar to Example 203. ¹H NMR (500 MHz, DMSO-d₆) δ=11.68 (s, 1H), 8.11 (d, J=7.4, 1H), 7.93 (s, 1H), 6.97 (m, 2H), 5.69 (s, 1H), 4.99 (s, 1H), 4.57 (s, 2H), 3.58 (m, 1H), 3.12-2.93 (m, 1H), 2.48-2.40 (m, 2H), 2.06-1.96 (m, 1H), 1.96-1.84 (m, 1H), 1.78-1.62 (m, 2H), 1.57 (m, 1H), 1.05-1.01 (m, 6H), 0.97-0.90 (m, 2H), 0.90-0.85 (m, 2H). LC-MS (ESI): m/z [M+H]⁺=478.3.

### Example 303: cis-3-(3-((2,2-dioxido-1,3-dihydrobenzo[c]isothiazol-6-yl)amino)-1H-pyrazol-5-yl)cyclopentyl isopropylcarbamate

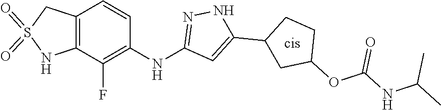 [SMILES: CC(C)NC(=O)OC1CCC(c2cc(Nc3ccc4c(c3F)NS(=O)(=O)C4)n[nH]2)C1]

The titled compound was synthesized in the procedures similar to Example 195 in racemic form, which was further separated by Chiral Prep-HPLC to give:

Enantiomer 1 (Example 303a, 100% ee); Retention time: 11.19 min. ¹H NMR (500 MHz, DMSO-d₆) δ 11.72 (s, 1H), 10.24 (s, 1H), 8.38 (s, 1H), 7.01 (d, J=8.3 Hz, 2H), 6.94 (d, J=7.4 Hz, 1H), 6.76 (d, J=6.7 Hz, 1H), 5.59 (s, 1H), 5.10-4.90 (m, 1H), 4.31 (s, 2H), 3.63-3.48 (m, 1H), 3.09-2.95 (m, 1H), 2.48-2.40 (m, 1H), 2.06-1.96 (m, 1H), 1.96-1.84 (m, 1H), 1.80-1.64 (m, 2H), 1.63-1.51 (m, 1H), 1.03 (d, J=6.3 Hz, 6H). LC-MS (ESI): m/z [M+H]⁺=420.2.

Enantiomer 2 (Example 303b, 94.18% ee); Retention time: 13.72 min. ¹H NMR (500 MHz, DMSO-d₆) δ 11.72 (s, 1H), 10.23 (s, 1H), 8.40 (s, 1H), 7.02 (d, J=8.3 Hz, 2H), 6.93 (s, 1H), 6.78 (d, J=5.6 Hz, 1H), 5.59 (s, 1H), 5.08-4.86 (m, 1H), 4.33 (s, 2H), 3.63-3.47 (m, 1H), 3.1-2.92 (m, 1H), 2.48-2.40 (m, 1H), 2.08-1.97 (m, 1H), 1.95-1.85 (m, 1H), 1.80-1.65 (m, 2H), 1.63-1.50 (m, 1H), 1.03 (d, J=5.3 Hz, 6H). LC-MS (ESI): m/z [M+H]⁺=420.3.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=20: 80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 304: cis-3-(3-((3,3-dimethyl-2,2-dioxido-1,3-dihydrobenzo[c]isothiazol-6-yl)amino)-1H-pyrazol-5-yl)cyclopentyl isopropylcarbamate

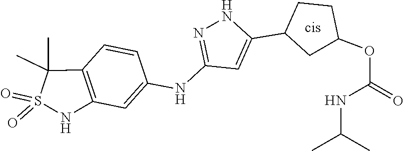 [SMILES: CC(C)NC(=O)OC1CCC(c2cc(Nc3ccc4c(c3)NS(=O)(=O)C4(C)C)n[nH]2)C1]

The titled compound was synthesized in the procedures similar to Example 194. ¹H NMR (500 MHz, DMSO-d₆) δ 11.72 (s, 1H), 10.15 (s, 1H), 8.38 (s, 1H), 7.77-7.65 (m, 1H), 7.06 (d, J=10 Hz, 1H), 7.04-6.91 (m, 1H), 6.84-6.75 (m, 1H), 5.59 (s, 1H), 5.04-4.95 (m, 1H), 3.64-3.51 (m, 1H), 3.08-2.97 (m, 1H), 2.45-2.41 (m, 1H), 2.04-1.97 (m, 1H), 1.95-1.85 (m, 1H), 1.77-1.65 (m, 2H), 1.63-1.54 (m, 1H), 1.48 (s, 6H), 1.09-0.98 (m, 6H). LC-MS (ESI): m/z [M+H]⁺=448.2.

### Example 305: cis-3-(3-((2,2-dioxido-1,3-dihydrobenzo[c]isothiazol-6-yl)amino)-1H-pyrazol-5-yl)cyclopentyl propylcarbamate

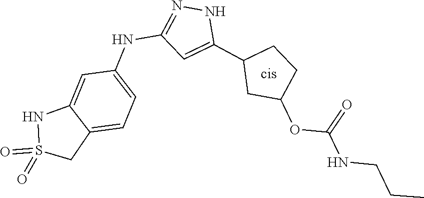 [SMILES: CCCNC(=O)OC1CCC(c2cc(Nc3ccc4c(c3)NS(=O)(=O)C4)n[nH]2)C1]

The titled compound was synthesized in the procedures similar to Example 195. ¹H NMR (500 MHz, DMSO-d₆) δ 11.72 (s, 1H), 10.22 (s, 1H), 8.38 (s, 1H), 7.88-6.98 (m, 3H), 6.77 (d, J=5 Hz, 1H), 5.60 (s, 1H), 5.03-4.95 (m, 1H), 4.33 (s, 2H), 3.09-2.98 (m, 1H), 2.95-2.85 (m, 2H), 2.46-2.40 (m, 1H), 2.06-1.96 (m, 1H), 1.95-1.85 (m, 1H), 1.78-1.65 (m, 2H), 1.63-1.55 (m, 1H), 1.43-1.32 (m, 2H), 0.81 (t, J=5 Hz, 3H). LC-MS (ESI): m/z [M+H]⁺=420.2.

### Example 306: cis-3-(3-((2,2-dioxido-1,3-dihydrobenzo[c]isothiazol-6-yl)amino)-1H-pyrazol-5-yl)cyclopentyl((S)-sec-butyl)carbamate

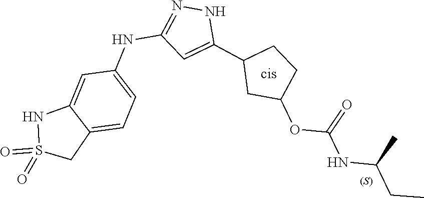

The titled compound was synthesized in the procedures similar to Example 195. ¹H NMR (500 MHz, DMSO-d₆) δ 11.77 (s, 1H), 10.22 (s, 1H), 8.38 (s, 1H), 7.04-6.99 (m, 2H), 6.87 (d, J=10 Hz, 1H), 6.80-6.75 (m, 1H), 5.60 (s, 1H), 5.03-4.95 (m, 1H), 4.33 (s, 2H), 3.42-3.34 (m, 1H), 3.08-2.98 (m, 1H), 2.47-2.42 (m, 1H), 2.06-1.96 (m, 1H), 1.95-1.85 (m, 1H), 1.77-1.66 (m, 2H), 1.64-1.55 (m, 1H), 1.41-1.28 (m, 2H), 1.04-0.96 (m, 3H), 0.85-0.75 (m, 3H). LC-MS (ESI): m/z [M+H]⁺=434.2.

### Example 307: cis-3-(3-((2,2-dioxido-1,3-dihydrobenzo[c]isothiazol-6-yl)amino)-1H-pyrazol-5-yl)cyclopentyl(1-methylcyclopropyl)carbamate

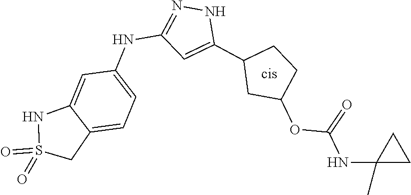 [SMILES: CC1(NC(=O)OC2CCC(c3cc(Nc4ccc5c(c4)NS(=O)(=O)C5)n[nH]3)C2)CC1]

The titled compound was synthesized in the procedures similar to Example 195. ¹H NMR (500 MHz, DMSO-d₆) δ 11.72 (s, 1H), 10.22 (s, 1H), 8.38 (s, 1H), 7.33 (s, 1H), 7.11-6.97 (m, 2H), 6.77 (d, J=10 Hz, 1H), 5.59 (s, 1H), 5.03-4.93 (m, 1H), 4.33 (s, 2H), 3.09-2.97 (m, 1H), 2.46-2.42 (m, 1H), 2.06-1.95 (m, 1H), 1.94-1.83 (m, 1H), 1.74-1.63 (m, 2H), 1.61-1.51 (m, 1H), 1.23 (s, 3H), 0.63-0.56 (m, 2H), 0.50-0.43 (m, 2H). LC-MS (ESI): m/z [M+H]⁺=432.2.

### Example 308: cis-3-(3-((1-methyl-2,2-dioxido-1,3-dihydrobenzo[c]isothiazol-6-yl)amino)-1H-pyrazol-5-yl)cyclopentyl isopropylcarbamate

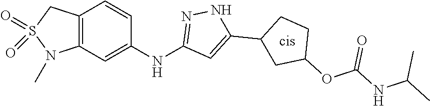 [SMILES: CC(C)NC(=O)OC1CCC(c2cc(Nc3ccc4c(c3)N(C)S(=O)(=O)C4)n[nH]2)C1]

The titled compound was synthesized in the procedures similar to Example 35. ¹H NMR (500 MHz, DMSO-d₆) δ 11.81 (s, 1H), 8.51 (s, 1H), 7.08 (d, J=8.2 Hz, 1H), 7.02 (s, 1H), 6.94 (d, J=7.2 Hz, 1H), 6.86 (d, J=7.9 Hz, 1H), 5.63 (s, 1H), 5.08-4.90 (m, 1H), 4.47 (s, 2H), 3.65-3.52 (m, 1H), 3.08-2.99 (m, 1H), 3.08-2.99 (m, 1H), 2.96 (s, 3H), 2.45-2.40 (m, 1H), 2.08-1.97 (m, 1H), 1.95-1.84 (m, 1H), 1.80-1.67 (m, 2H), 1.64-1.55 (m, 1H), 1.03 (d, J=6.3 Hz, 6H). LC-MS (ESI): m/z [M+H]⁺=434.3.

### Example 309: cis-3-(3-((1-(2-hydroxyethyl)-2,2-dioxido-1,3-dihydrobenzo[c]isothiazol-6-yl)amino)-1H-pyrazol-5-yl)cyclopentyl isopropylcarbamate

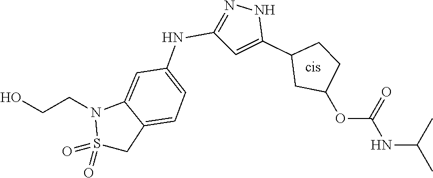 [SMILES: CC(C)NC(=O)OC1CCC(c2cc(Nc3ccc4c(c3)N(CCO)S(=O)(=O)C4)n[nH]2)C1]

The titled compound was synthesized in the procedures similar to Example 271. ¹H NMR (500 MHz, DMSO-d₆) δ 11.83 (s, 1H), 8.47 (s, 1H), 7.06 (d, J=8.0 Hz, 2H), 6.94 (d, J=7.3 Hz, 1H), 6.87 (d, J=8.0 Hz, 1H), 5.62 (s, 1H), 4.98 (s, 1H), 4.45 (s, 2H), 3.65 (t, J=6.5 Hz, 2H), 3.61-3.54 (m, 1H), 3.50 (t, J=6.5 Hz, 3H), 3.07-2.96 (m, 1H), 2.50-2.44 (m, 2H), 2.03-1.98 (m, 1H), 1.96-1.85 (m, 1H), 1.71-1.65 (m, 2H), 1.58-1.48 (m, 1H), 1.03 (d, J=6.1 Hz, 6H). LC-MS (ESI): m/z [M+H]⁺=464.2.

### Example 310: cis-3-(3-((5-fluoro-2,2-dioxido-1,3-dihydrobenzo[c]isothiazol-6-yl)amino)-1H-pyrazol-5-yl)cyclopentyl isopropylcarbamate

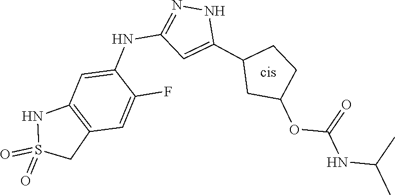 [SMILES: CC(C)NC(=O)OC1CCC(c2cc(Nc3cc4c(cc3F)CS(=O)(=O)N4)n[nH]2)C1]

The titled compound was synthesized in the procedures similar to Example 259. ¹H NMR (500 MHz, DMSO-d₆) δ 11.81 (s, 1H), 10.20 (s, 1H), 8.16 (s, 1H), 7.73 (s, 1H), 7.08 (d, J=11.3 Hz, 1H), 6.92 (s, 1H), 5.75 (s, 1H), 5.00 (s, 1H), 4.37 (s, 2H), 3.62-3.51 (m, 1H), 3.09-3.02 (m, 1H), 2.48-2.42 (m, 1H), 2.05-1.85 (m, 2H), 1.78-1.55 (m, 3H), 1.03 (d, J=6.3 Hz, 6H). LC-MS (ESI): m/z [M+H]⁺=438.2.

### Example 311: cis-3-(3-((4-fluoro-2,2-dioxido-1,3-dihydrobenzo[c]isothiazol-6-yl)amino)-1H-pyrazol-5-yl)cyclopentyl isopropylcarbamate

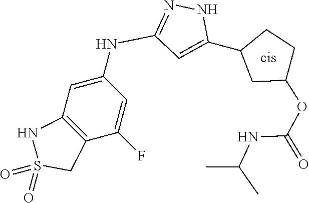 [SMILES: CC(C)NC(=O)OC1CCC(c2cc(Nc3cc(F)c4c(c3)NS(=O)(=O)C4)n[nH]2)C1]

The titled compound was synthesized in the procedures similar to Example 203. ¹H NMR (500 MHz, DMSO-d₆) δ 11.85 (s, 1H), 9.05 (s, 1H), 7.79 (t, J=4.8 Hz, 1H), 7.60 (s, 1H), 7.32 (d, J=11.9 Hz, 1H), 6.88 (d, J=7.4 Hz, 1H), 5.57 (s, 1H), 4.93 (s, 1H), 4.45 (s, 2H), 3.51 (dd, J=12.6, 6.4 Hz, 1H), 3.13-2.88 (m, 1H), 2.41-2.33 (m, 1H), 2.03-1.91 (m, 1H), 1.90-1.78 (m, 1H), 1.70-1.60 (m, 2H), 1.58-1.48 (m, 1H), 0.96 (d, J=6.2 Hz, 6H). LC-MS (ESI): m/z [M+H]⁺=438.2.

### Example 312: cis-3-(3-((7-fluoro-2,2-dioxido-1,3-dihydrobenzo[c]isothiazol-6-yl)amino)-1H-pyrazol-5-yl)cyclopentyl isopropylcarbamate

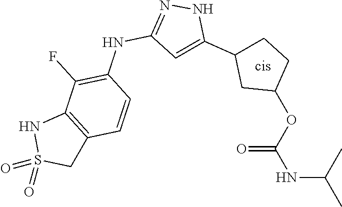 [SMILES: CC(C)NC(=O)OC1CCC(c2cc(Nc3ccc4c(c3F)NS(=O)(=O)C4)n[nH]2)C1]

The titled compound was synthesized in the procedures similar to Example 203. ¹H NMR (500 MHz, DMSO-d₆) δ 11.82 (s, 1H), 10.64 (s, 1H), 8.13 (d, J=7.9 Hz, 1H), 7.60 (s, 1H), 6.93 (d, J=8.3 Hz, 2H), 5.74 (s, 1H), 4.99 (s, 1H), 4.47 (s, 2H), 3.58 (dd, J=13.2, 6.5 Hz, 1H), 3.13-2.96 (m, 1H), 2.45 (dd, J=13.8, 7.1 Hz, 1H), 2.01 (dd, J=15.8, 7.4 Hz, 1H), 1.95-1.85 (m, 1H), 1.70 (dd, J=19.2, 10.0 Hz, 2H), 1.60 (d, J=13.5 Hz, 1H), 1.03 (d, J=6.3 Hz, 6H). LC-MS (ESI): m/z [M+H]⁺=438.2.

### Example 313: cis-3-(3-((6-fluoro-1,1-dioxido-2,3-dihydrobenzo[b]thiophen-5-yl)amino)-1H-pyrazol-5-yl)cyclopentyl isopropylcarbamate

### Step 1:(4-bromo-3-fluorophenyl) (2,2-diethoxyethyl) sulfane

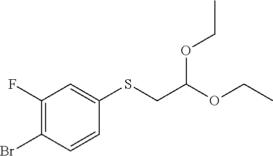 [SMILES: CCOC(CSc1ccc(Br)c(F)c1)OCC]

To a solution of 4-bromo-3-fluorobenzenethiol (2.0 g, 9.7 mmol) and 2-bromo-1,1-diethoxyethane (2.87 g, 14.56 mmol) in DMF (20 mL) was added K₂CO₃ (2.0 g, 14.56 mmol), the reaction mixture was stirred for 7 hours at 135° C. under nitrogen atmosphere. The resulting mixture was cooled to room temperature, diluted with water (100 mL), extracted with EtOAc (3×100 mL), the combined organic phase was washed with water, then brine, dried over Na₂SO₄, filtered, concentrated under reduce pressure, dried over in vacuo, to afford the crude product. (3.0 g crude). ¹H NMR (500 MHz, DMSO-d₆) δ=7.59 (dd, J=8.3, 7.7, 1H), 7.42 (dd, J=9.9, 2.2, 1H), 7.14 (dd, J=8.4, 1.9, 1H), 4.63 (t, J=5.4, 1H), 3.60 (m, 2H), 3.49 (m, 2H), 3.20 (d, J=5.5, 2H), 1.09 (t, J=7.0, 6H).

### Step 2:5-bromo-6-fluorobenzo[b]thiophene & 5-bromo-4-fluorobenzo[b]thiophene

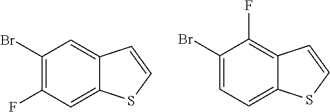 [SMILES: Fc1cc2sccc2cc1Br ; Fc1c(Br)ccc2sccc12]

To a solution of (4-bromo-3-fluorophenyl) (2,2-diethoxyethyl) sulfane (2.0 g, 6.21 mmol) in PhCl (60 mL) was added polyphosphoric acid (3.6 g), the reaction mixture was stirred for 16 hours at 130° C. The resulting mixture was cooled to room temperature, diluted with water (100 mL), extracted with EtOAc (3×100 mL), the combined organic phase was washed with saturated Na₂CO₃ aqueous, then brine, dried over Na₂SO₄, filtered, concentrated under reduce pressure, purified by silica gel column chromatography, eluting with PE to afford the product (1.2 g crude, 5-bromo-4-fluorobenzo[b]thiophene: 5-bromo-6-fluorobenzo[b]thiophene=1:4).

### Step 3:5-bromo-6-fluorobenzo[b]thiophene 1,1-dioxide

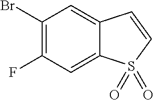 [SMILES: O=S1(=O)C=Cc2cc(Br)c(F)cc21]

A mix of 5-bromo-4-fluorobenzo[b]thiophene and 5-bromo-6-fluorobenzo[b]thiophene (956 mg crude, 4.156 mmol, 1:4) was dissolved in DCM (60 mL), m-CPBA (2.86 g, 12.47 mmol) was added one portion, the reaction mixture was stirred for 16 hours at room temperature. The resulting mixture was washed with saturated Na₂CO₃ aqueous, the brine, dried over Na₂SO₄, filtered, concentrated under reduce pressure, purified by silica gel column chromatography, eluting with PE/EA (4:1) to afford the product (420 mg, 30% for 2 steps). LC-MS (ESI): m/z [M+H]⁺=263.1.

### Step 4:5-bromo-6-fluoro-2,3-dihydrobenzo[b]thiophene 1,1-dioxide

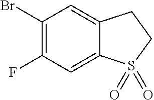 [SMILES: O=S1(=O)CCc2cc(Br)c(F)cc21]

To a solution of 5-bromo-6-fluorobenzo[b]thiophene 1,1-dioxide (420 mg, 1.7 mmol) in EtOH (10 mL) was added NaBH₄ (129 mg, 3.4 mmol), the reaction mixture was stirred for 6 hours at room temperature. The resulting mixture was concentrated, purified by prep-TLC (PE/EA=4:1) to afford the product (251 mg, 56%). LC-MS (ESI): m/z [M+H]⁺=265.0.

### Step 5: cis-3-(3-((6-fluoro-1,1-dioxido-2,3-dihydrobenzo[b]thiophen-5-yl)amino)-1H-pyrazol-5-yl)cyclopentyl isopropylcarbamate

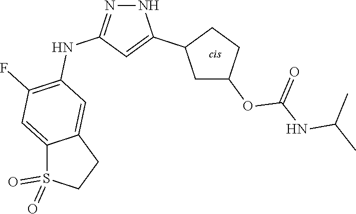 [SMILES: CC(C)NC(=O)OC1CCC(c2cc(Nc3cc4c(cc3F)S(=O)(=O)CC4)n[nH]2)C1]

The titled compound was synthesized in the procedures similar to Example 34, step 5. ¹H NMR (500 MHz, DMSO-d₆) δ 11.99 (s, 1H), 8.74 (s, 1H), 8.14 (s, 1H), 7.54 (d, J=10.1 Hz, 1H), 6.95 (d, J=7.3 Hz, 1H), 5.85 (s, 1H), 5.00 (s, 1H), 3.64-3.51 (m, 3H), 3.25 (t, J=6.7 Hz, 2H), 3.11-3.01 (m, 1H), 2.45 (d, J=7.3 Hz, OH), 2.09-1.98 (m, 1H), 1.96-1.84 (m, 1H), 1.78-1.65 (m, 2H), 1.63-1.53 (m, 1H), 1.03 (d, J=6.1 Hz, 6H). LC-MS (ESI): m/z [M+H]⁺=437.2.

### Example 314: cis-3-(3-((7-fluoro-1,1-dioxido-2,3-dihydrobenzo[b]thiophen-6-yl)amino)-1H-pyrazol-5-yl)cyclopentyl isopropylcarbamate

### Step 1: S-(3-bromo-2-fluorophenyl)ethanethioate

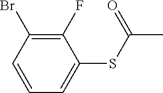

A mixture of 1-bromo-2-fluoro-3-iodobenzene (6 g, 20 mmol), Potassium thioacetate (4.57 g, 40 mmol), o-Phenanthroline (7.2 g, 40 mmol) and CuI (0.6 g, 10% wt), in PhMe (100 mL) was stirred for 3 h at 100° C. under N₂ atmosphere. The resulting mixture was concentrated under reduced pressure. The residue was purified by silica gel column chromatography, eluting with PE/EtOAc (5:1) to afford the product (3 g, 60.5%). LC-MS (ESI): m/z [M+H]⁺=249.5.

### Step 2:3-bromo-2-fluorobenzenethiol

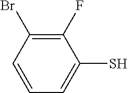

A mixture of S-(3-bromo-2-fluorophenyl)ethanethioate (3 g, 12 mmol) and KOH (0.81 g, 14.4 mmol) in THF/MeOH (1:1, 50 mL) was stirred for 16 h at 20° C. The mixture acided to pH=6 with saturated HCl (aq. 10%) and extracted with EA (100 mL×2). The organic phase was concentrated under reduced pressure. The mixture was concentrated under reduced pressure. The residue was purified by silica gel column chromatography, eluting with PE/EtOAc (1:1) to afford the product (1 g, 40.2%), LC-MS (ESI): m/z [M+H]⁺=207.4.

### Step 3: (3-bromo-2-fluorophenyl) (2,2-diethoxyethyl) sulfane

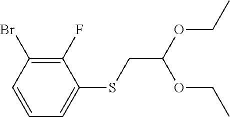 [SMILES: CCOC(CSc1cccc(Br)c1F)OCC]

A solution of 3-bromo-2-fluorobenzenethiol (1 g, 4.8 mmol), 2-bromo-1,1-diethoxyethane (1.4 g, 7.2 mmol) and K₂CO₃ (2 g, 14.4 mmol) in DMF (20 mL) was stirred at 135° C. for 16 h. The solution was concentrated under reduced pressure. The residue was purified by silica gel column chromatography, eluting with PE/EtOAc (4:1) to afford the product (1.3 g, 84%), [M+H]⁺=323.2.

### Step 4:6-bromo-7-fluorobenzo[b]thiophene

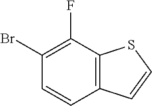 [SMILES: Fc1c(Br)ccc2ccsc12]

A solution of (3-bromo-2-fluorophenyl) (2,2-diethoxyethyl) sulfane (1.3 g, 4 mmol) and polyphosphoric acid (10 mL) in PhCl₃ (50 mL) was stirred at 130° C. for 16 h. The solution was concentrated under reduced pressure. The residue was purified by silica gel column chromatography, eluting with PE/EtOAc (3:1) to afford the product (0.5 g, 52.5%), LC-MS (ESI): m/z [M+H]⁺=231.4.

### Step 5:6-bromo-7-fluorobenzo[b]thiophene 1,1-dioxide

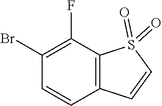 [SMILES: O=S1(=O)C=Cc2ccc(Br)c(F)c21]

A solution of 6-bromo-7-fluorobenzo[b]thiophene (0.5 g, 2.1 mmol) and m-CPBA (1.1 g, 6.3 mmol) in DCM (20 mL) was stirred at 20° C. for 16 h. The solution was concentrated under reduced pressure. The residue was purified by silica gel column chromatography, eluting with PE/EtOAc (3:1) to afford the product (0.5 g, 90% 1.9 mmol), LC-MS (ESI): m/z [M+H]⁺=263.3.

### Step 6:6-bromo-7-fluoro-2,3-dihydrobenzo[b]thiophene 1,1-dioxide

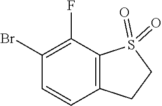 [SMILES: O=S1(=O)CCc2ccc(Br)c(F)c21]

A solution of 6-bromo-7-fluorobenzo[b]thiophene 1,1-dioxide (0.5 g, 1.9 mmol) and NaBH₄ (145 g, 3.8 mol) in EtOH (10 mL) was stirred at 20° C. for 16 h. The solution was concentrated under reduced pressure. The residue was purified by silica gel column chromatography, eluting with PE/EtOAc (3:1) to afford the product (0.4 g, 79%), LC-MS (ESI): m/z [M+H]⁺=265.4.

### Step 7: cis-3-(3-((7-fluoro-1,1-dioxido-2,3-dihydrobenzo[b]thiophen-6-yl)amino)-1H-pyrazol-5-yl)cyclopentyl isopropylcarbamate

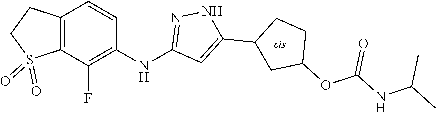 [SMILES: CC(C)NC(=O)OC1CCC(c2cc(Nc3ccc4c(c3F)S(=O)(=O)CC4)n[nH]2)C1]

A mixture of 6-bromo-7-fluoro-2,3-dihydrobenzo[b]thiophene 1,1-dioxide (100 mg, 0.37 mmol), cis-3-(3-amino-1H-pyrazol-5-yl)cyclopentyl isopropylcarbamate (93 mg, 0.37 mmol), Brettephos Pd G3 (36 mg, 0.04 mmol) and K₂CO₃ (153 mg, 1.11 mmol) in t-BuOH (15 mL) was stirred for 16 h at 110° C. in the sealed tube under N₂ atmosphere. The solution was concentrated under reduced pressure. The residue was purified by silica gel column chromatography, eluting with PE/EtOAc (1:4) to afford the crude product (0.1 g). Then the crude product was further purified by prep HPLC (Waters XSelect C18: RD-CO-094 column, eluting with a gradient of acetonitrile/water containing 0.1% FA, 20%-55%) to afford the product (80 mg, 48.6%). ¹H NMR (500 MHz, DMSO-d₆) δ 11.87 (s, 1H), 8.43 (s, 1H), 8.40-8.27 (m, 1H), 7.16 (d, J=8.5 Hz, 1H), 6.94 (d, J=7.4 Hz, 1H), 5.75 (s, 1H), 5.10-4.90 (m, 1H), 3.70-3.63 (m, 2H), 3.62-3.53 (m, 1H), 3.25 (t, J=6.9 Hz, 2H), 3.09-2.99 (m, 1H), 2.48-2.42 (m, 1H), 2.07-1.97 (m, 1H), 1.96-1.83 (m, 1H), 1.78-1.67 (m, 2H), 1.65-1.53 (m, 1H), 1.03 (d, J=6.3 Hz, 6H). LC-MS (ESI): m/z [M+H]⁺=437.32.

### Example 315: cis-3-(3-((4-fluoro-1,1-dioxido-2,3-dihydrobenzo[b]thiophen-5-yl)amino)-1H-pyrazol-5-yl)cyclopentyl isopropylcarbamate

### Step 1:5-bromo-4-fluorobenzo[b]thiophene

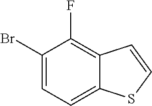 [SMILES: Fc1c(Br)ccc2sccc12]

To a solution of (4-bromo-3-fluorophenyl) (2,2-diethoxyethyl) sulfane (2.0 g, 6.21 mmol) in PhCl (60 mL) was added polyphosphoric acid (3.6 g), the reaction mixture was stirred for 16 hours at 130° C. The resulting mixture was cooled to room temperature, diluted with water (100 mL), extracted with EtOAc (3*100 mL), the combined organic phase was washed with saturated Na₂CO₃ aqueous, then brine, dried over Na₂SO₄, filtered, concentrated under reduce pressure, purified by silica gel column chromatography, eluting with PE to afford the product (1.2 g crude, 5-bromo-4-fluorobenzo[b]thiophene: 5-bromo-6-fluorobenzo[b]thiophene=1:4).

### Step 2:5-bromo-4-fluorobenzo[b]thiophene 1,1-dioxide

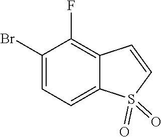 [SMILES: O=S1(=O)C=Cc2c1ccc(Br)c2F]

To a solution of 5-bromo-4-fluorobenzo[b]thiophene and 5-bromo-6-fluorobenzo[b]thiophene (956 mg crude, 4.156 mmol, isomers 1:4) was dissolved in DCM (60 mL), m-CPBA (2.86 g, 12.47 mmol) was added one portion, the reaction mixture was stirred for 16 hours at room temperature. The resulting mixture was washed with saturated Na₂CO₃ aqueous, the brine, dried over Na₂SO₄, filtered, concentrated under reduce pressure, purified by silica gel column chromatography, eluting with PE/EA (3:1) to afford the product (110 mg, 7% for 2 steps). LC-MS (ESI): m/z [M+H]⁺=263.1;

### Step 3:5-bromo-4-fluoro-2,3-dihydrobenzo[b]thiophene 1,1-dioxide

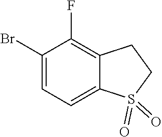 [SMILES: O=S1(=O)CCc2c1ccc(Br)c2F]

To a solution of 5-bromo-4-fluorobenzo[b]thiophene 1,1-dioxide (110 mg, 0.42 mmol) in EtOH (5 mL) was added NaBH₄ (48 mg, 1.26 mmol), the reaction mixture was stirred for 6 hours at room temperature. The resulting mixture was concentrated, purified by prep-TLC (PE/EA=4:1) to afford the product (100 mg, 91%). ¹H NMR (500 MHz, DMSO-d₆) δ=7.90 (dd, J=8.1, 6.1, 1H), 7.59 (d, J=8.2, 1H), 3.71-3.65 (m, 2H), 3.40 (t, J=6.8, 2H). LC-MS (ESI): m/z [M+H]⁺=265.0.

### Step 4: cis-3-(3-((4-fluoro-1,1-dioxido-2,3-dihydrobenzo[b]thiophen-5-yl)amino)-1H-pyrazol-5-yl)cyclopentyl isopropylcarbamate

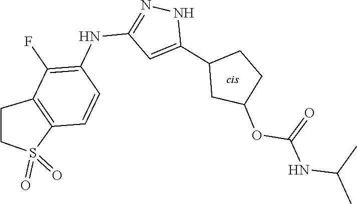 [SMILES: CC(C)NC(=O)OC1CCC(c2cc(Nc3ccc4c(c3F)CCS4(=O)=O)n[nH]2)C1]

To a solution of 5-bromo-4-fluoro-2,3-dihydrobenzo[b]thiophene 1,1-dioxide (100 mg, 0.38 mmol) and cis-3-(3-amino-1H-pyrazol-5-yl)cyclopentyl isopropylcarbamate (100 mg, 0.38 mmol) in t-BuOH (10 mL) was added BrettPhos Pd G3 (34 mg, 0.038 mmol) and K₂CO₃ (160 mg, 1.16 mmol), the reaction mixture was stirred for 16 hours at 110° C. under nitrogen atmosphere. The resulting mixture was cooled to room temperature, filtered, the filtrate was concentrated under reduce pressure, purified by silica gel column chromatography, eluting with PE/EA (1:4) to afford the product (150 mg, 90%) in a racemic form, which was further separated by Chiral Prep-HPLC to give:

Enantiomer 1 (Example 315a, 100% ee); Retention time: 5.27 min. ¹H NMR (500 MHz, DMSO-d₆) δ=11.98 (s, 1H), 8.70 (s, 1H), 8.23 (t, J=8.1, 1H), 7.41 (d, J=8.6, 1H), 6.94 (d, J=7.4, 1H), 5.83 (d, J=2.0, 1H), 5.00 (s, 1H), 3.56 (t, J=6.9, 3H), 3.33 (m, 2H), 3.12-2.98 (m, 1H), 2.48-2.41 (m, 1H), 2.07-1.98 (m, 1H), 1.98-1.86 (m, 1H), 1.78-1.65 (m, 2H), 1.59 (m, 1H), 1.08-0.95 (m, 6H). LC-MS (ESI): m/z [M+H]⁺=437.35.

Enantiomer 2 (Example 315b, 100% ee); Retention time: 9.341 min. ¹H NMR (500 MHz, DMSO-d₆) δ=11.98 (s, 1H), 8.70 (s, 1H), 8.23 (s, 1H), 7.53-7.33 (m, 1H), 6.95 (s, 1H), 5.84 (m, 1H), 5.00 (s, 1H), 3.65-3.45 (m, 3H), 3.30-3.22 (m, 2H), 3.08 (m, 1H), 2.45-2.38 (m, 1H), 2.03 (m, 1H), 1.91 (m, 1H), 1.73 (m, 2H), 1.61 (m, 1H), 1.04 (m, 6H). LC-MS (ESI): m/z [M+H]⁺=437.35.

Chiral analytical method: Column: CHIRALPAK IE 4.6×250 mm 5 μm; Mobile phase: A for MtBE (0.1% 2M NH₃ MeOH) and B for MeOH: DCM=50:50 (v/v); Gradient: Mobile Phase A: Mobile Phase B=10: 90 (v/v); HPLC Equipment: HPLC-Agilent; Column temperature: 35° C.

Chiral Prep-HPLC Condition: IE 21.2 mm×250 mm, 5 μm; Mobile phase: A for MtBE and B for DCM: MeOH (0.2% 2M NH₃. MeOH); Gradient: Mobile Phase A: Mobile Phase B=50: 50 (v/v); Flow Rate: 18 mL/min, Wavelength: UV 280 nm and 300 nm, Prep-HPLC Equipment: Prep-HPLC-Gilson; Column temperature: 25° C.

### Example 316: cis-(3-((2,2-dioxido-1,3-dihydrobenzo[c]thiophen-5-yl)amino)-1H-pyrazol-5-yl)cyclopentyl isopropylcarbamate

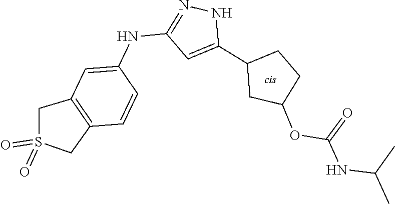 [SMILES: CC(C)NC(=O)OC1CCC(c2cc(Nc3ccc4c(c3)CS(=O)(=O)C4)n[nH]2)C1]

¹H NMR (500 MHz, DMSO-d₆) δ 11.76 (s, 1H), 8.47 (s, 1H), 7.39 (s, 1H), 7.23-7.19 (m, 1H), 7.13 (d, J=8.4 Hz, 1H), 6.95 (d, J=7.2 Hz, 1H), 5.63 (s, 1H), 4.98 (s, 1H), 4.40 (s, 2H), 4.32 (s, 2H), 3.63-3.51 (m, 1H), 3.11-2.98 (m, 1H), 2.48-2.38 (m, 1H), 2.05-1.97 (m, 1H), 1.95-1.81 (m, 1H), 1.78-1.64 (m, 2H), 1.63-1.53 (m, 1H), 1.03 (d, J=6.4 Hz, 6H). LC-MS (ESI): m/z [M+H]⁺=419.3.

### Example 317: cis-3-(3-((4-fluoro-2,2-dioxido-1,3-dihydrobenzo[c]thiophen-5-yl)amino)-1H-pyrazol-5-yl)cyclopentyl isopropylcarbamate

### Step 1:4-bromo-3-fluorophthalic acid

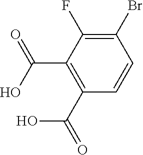 [SMILES: O=C(O)c1ccc(Br)c(F)c1C(=O)O]

To a solution of 4-bromo-3-fluorobenzoic acid (10 g, 45.8 mmol) in THF (100 mL) were added LDA (51 mL, 100.9 mmol) at −78° C. under nitrogen atmosphere for 1 h. The dry CO₂ (10 g) was added in it at −78° C. for 1 h. The reaction was quenched with NH₄Cl solution (80 mL). The resulting solution was extracted with 3×150 mL of DCM. The combined organic phases were concentrated under reduced pressure. The residue was the crude product (10 g, 83%). LCMS (ESI) m/z [M−H]⁻=261/263.

### Step 2: (4-bromo-3-fluoro-1,2-phenylene) dimethanol

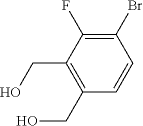 [SMILES: OCc1ccc(Br)c(F)c1CO]

To a solution of 4-bromo-3-fluorophthalic acid (10 g, 38.1 mmol) in THF (200 mL) was added BH₃-THF (110 mL, 114.3 mmol) at 0° C. The resulting solution was stirred for overnight at 50° C. The reaction was then quenched with MeOH (150 mL) at 0° C. The resulting mixture was concentrated under vacuum. The residue was purified by combi-flash (DCM/MeOH=80%: 20%) to give the product (8.2 g, 94.2%) as colorless oil. LCMS (ESI) m/z [M−H]⁻=233/235.

### Step 3:1-bromo-3,4-bis(chloromethyl)-2-fluorobenzene

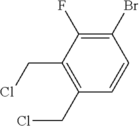 [SMILES: Fc1c(Br)ccc(CCl)c1CCl]

To a solution of (4-bromo-3-fluoro-1,2-phenylene) dimethanol (4 g, 17.1 mmol) in SOCl₂ (40 mL) at 0° C. The resulting solution was stirred for 3 h at 80° C. The resulting mixture was concentrated under vacuum. The reaction was then quenched with ice-water (50 mL). The mixture was acidified to PH>8 with NaOH. The resulting solution was extracted with 3×100 mL of DCM. The resulting mixture was concentrated under vacuum. The residue was the crude product (4.4 g, 95.4%).

### Step 4:5-bromo-4-fluoro-1,3-dihydrobenzo[c]thiophene

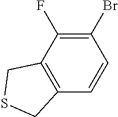 [SMILES: Fc1c(Br)ccc2c1CSC2]

To a solution of 1-bromo-3,4-bis(chloromethyl)-2-fluorobenzene (4.4 g, 16.2 mmol) in EtOH (40 mL) was added Na₂S 9H₂O (6.06 g, 25.3 mmol) and H₂O (10 mL). The resulting solution was stirred at 80° C. for overnight. The resulting mixture was concentrated under vacuum. The resulting solution was extracted with 3×80 mL of DCM. The organic layers was washed with brine. The residue was purified by combi-flash (EtOAc/PE=2%: 98%) to give the product (600 mg, 15.8%).

### Step 5:5-bromo-4-fluoro-1,3-dihydrobenzo[c]thiophene 2,2-dioxide

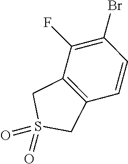 [SMILES: O=S1(=O)Cc2ccc(Br)c(F)c2C1]

To a solution of 5-bromo-4-fluoro-1,3-dihydrobenzo[c]thiophene (600 mg, 2.6 mmol) in THF: H₂O=4:1 (80 mL) were added NaIO₄ (1.67 g, 7.7 mmol), RuCl₃ (58.5 mg, 0.26 mmol) at 0° C. The resulting solution was stirred for 2 h at rt. The resulting solution was extracted with 3×50 mL of EtOAc. The resulting mixture was concentrated under vacuum. The residue was purified by combi-flash (EtOAc/PE=7: 93%) to give the product (427 mg, 62.6%).

### Step 6: cis-3-(3-((4-fluoro-2,2-dioxido-1,3-dihydrobenzo[c]thiophen-5-yl)amino)-1H-pyrazol-5-yl)cyclopentyl isopropylcarbamate

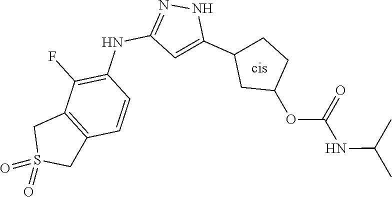 [SMILES: CC(C)NC(=O)OC1CCC(c2cc(Nc3ccc4c(c3F)CS(=O)(=O)C4)n[nH]2)C1]

The titled compound was synthesized in the procedures similar to Example 34. ¹H NMR (500 MHz, DMSO-d₆) δ=11.81 (s, 1H), 8.20 (s, 1H), 8.08 (t, J=8.5, 1H), 7.05 (d, J=8.5, 1H), 6.94 (d, J=7.5, 1H), 5.75 (s, 1H), 4.99 (s, 1H), 4.51 (s, 2H), 4.42 (s, 2H), 3.58 (m, 1H), 3.11-2.93 (m, 1H), 2.45 (m, 1H), 2.06-1.96 (m, 1H), 1.96-1.83 (m, 1H), 1.70 (m, 2H), 1.58 (m, 1H), 1.03 (m, 6H). LC-MS (ESI): m/z [M+H]⁺=437.35.

### Example 318: cis-tert-butyl 6-((5-(3-((isopropylcarbamoyl)oxy) cyclopentyl)-1H-pyrazol-3-yl)amino)benzo[d]thiazole-3 (2H)-carboxylate 1,1-dioxide

### Step 1:6-bromo-2,3-dihydrobenzo[d]thiazole

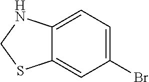

Into a 500-mL round-bottom flask were added 6-bromobenzo[d]thiazole (6.00 g, 28.17 mmol), Ammonia borane (1.31 g, 41.86 mmol), Tris(pentafluorophenyl) borate (1.44 g, 2.81 mmol) and Toluene (100 mL) at room temperature. The resulting mixture was stirred 8 h at 80° C. under nitrogen atmosphere. The resulting mixture was concentrated under reduced pressure. The residue was purified by silica gel column chromatography, eluted with PE/EA (100: 0~80:20) to afford 6-bromo-2,3-dihydrobenzo[d]thiazole (3.50 g, 57.79%). LC-MS (ESI): m/z [M+H]⁺=215.90.

### Step 2: tert-butyl 6-bromobenzo[d]thiazole-3 (2H)-carboxylate

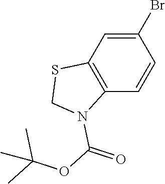 [SMILES: CC(C)(C)OC(=O)N1CSc2cc(Br)ccc21]

Into a 250-mL round-bottom flask were added 6-bromo-2,3-dihydrobenzo[d]thiazole (3.50 g, 16.28 mmol), Boc₂O (6.90 g, 32.56 mmol), TEA (4.93 g, 48.84 mmol), DMAP (397.7 mg, 3.26 mmol), DCM (70 mL) at room temperature. The resulting mixture was stirred 2 h at room temperature. The resulting mixture was extracted with DCM (2× 200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (100: 0~95:5) to afford tert-butyl 6-bromobenzo[d]thiazole-3 (2H)-carboxylate (3.10 g, 60.45%). LC-MS (ESI): m/z [M+Na]⁺=338.5.

### Step 3: tert-butyl 6-bromobenzo[d]thiazole-3 (2H)-carboxylate 1,1-dioxide

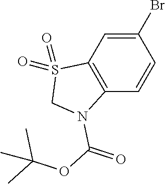 [SMILES: CC(C)(C)OC(=O)N1CS(=O)(=O)c2cc(Br)ccc21]

Into a 250-mL round-bottom flask were added tert-butyl 6-bromobenzo[d]thiazole-3 (2H)-carboxylate (3.00 g, 9.52 mmol), MCPBA (3.29 g, 19.04 mmol) and AcOH (20 mL) at room temperature. The resulting mixture was stirred 2 d at 30° C. under nitrogen atmosphere. The resulting mixture was extracted with EtOAc (2×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (100: 0~70:30) to afford tert-butyl 6-bromobenzo[d]thiazole-3 (2H)-carboxylate 1,1-dioxide (1.32 g, 39.94%). LC-MS (ESI): m/z [M+Na]⁺=370.4.

### Step 4: cis-tert-butyl 6-((5-(3-((isopropylcarbamoyl)oxy) cyclopentyl)-1H-pyrazol-3-yl)amino)benzo[d]thiazole-3 (2H)-carboxylate 1,1-dioxide

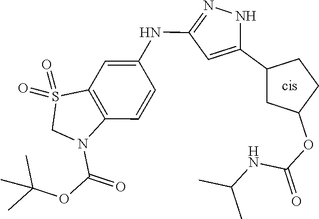 [SMILES: CC(C)NC(=O)OC1CCC(c2cc(Nc3ccc4c(c3)S(=O)(=O)CN4C(=O)OC(C)(C)C)n[nH]2)C1]

The titled compound was synthesized in the procedures similar to Example 34. ¹H NMR (500 MHz, DMSO-d₆) δ=11.81 (s, 1H), 8.83 (s, 1H), 8.04 (s, 1H), 7.87 (s, 1H), 7.42 (d, J=9.1, 1H), 6.95 (d, J=7.1, 1H), 5.60 (s, 1H), 5.03 (s, 2H), 5.00 (m, 1H), 3.58 (m, 1H), 3.11-3.01 (m, 1H), 2.43 (m, 1H), 2.02 (m, 1H), 1.96-1.83 (m, 1H), 1.72 (m, 2H), 1.59 (m, 1H), 1.52 (s, 9H), 1.04 (m, 6H). LC-MS (ESI): m/z [M+H]⁺=520.3.

### Example 319: cis-tert-butyl 5-((5-(3-((isopropylcarbamoyl)oxy) cyclopentyl)-1H-pyrazol-3-yl)amino)benzo[d]thiazole-3 (2H)-carboxylate 1,1-dioxide

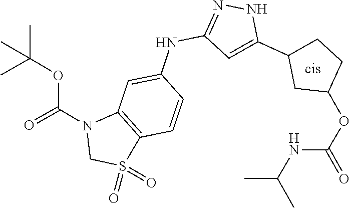 [SMILES: CC(C)NC(=O)OC1CCC(c2cc(Nc3ccc4c(c3)N(C(=O)OC(C)(C)C)CS4(=O)=O)n[nH]2)C1]

The titled compound was synthesized in the procedures similar to Example 318. ¹H NMR (500 MHz, DMSO-d₆) δ=12.01 (s, 1H), 9.11 (s, 1H), 8.10 (s, 1H), 7.52 (d, J=8.7, 1H), 7.15 (s, 1H), 6.94 (d, J=6.3, 1H), 5.69 (s, 1H), 4.99 (s, 1H), 4.95 (s, 2H), 3.57 (m, 1H), 3.05 (m, 1H), 2.44 (m, 1H), 2.02 (m, 1H), 1.90 (m, 1H), 1.71 (m, 2H), 1.60 (m, 1H), 1.52 (s, 9H), 1.03 (m, 6H). LC-MS (ESI): m/z [M+H]⁺=520.36.

### Example 320: cis-3-(3-((2,2-dioxido-1,3-dihydrobenzo[c][1,2,5]thiadiazol-5-yl)amino)-1H-pyrazol-5-yl)cyclopentyl isopropylcarbamate

### Step 1:5-bromo-1,3-dihydrobenzo[c][1,2,5]thiadiazole 2,2-dioxide

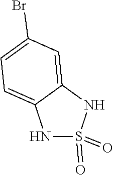 [SMILES: O=S1(=O)Nc2ccc(Br)cc2N1]

A solution of 4-bromobenzene-1,2-diamine (1 g, 5.3 mmol) and Sulfamide (0.8 g, 8.3 mmol) in diglyme (30 mL) was stirred at 160° C. for 16 h. The solution was concentrated under reduced pressure. The residue was purified by silica gel column chromatography, eluting with DCM/MeOH (10:1) to afford the product (0.5 g, 38%), LC-MS (ESI): m/z [M+H]⁺=249.5.

### Step 2:5-bromo-1,3-bis(4-methoxybenzyl)-1,3-dihydrobenzo[c][1,2,5]thiadiazole 2,2-dioxide

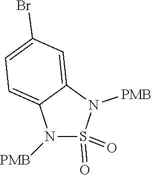

A mixture of 5-bromo-1,3-dihydrobenzo[c][1,2,5]thiadiazole 2,2-dioxide (0.2 g, 0.8 mmol), 4-Methoxybenzyl bromide (0.8 g, 4 mmol) and K₂CO₃ (0.55 g, 4 mmol) in DMF (10 mL) was stirred at 20° C. for 3 h. The solution was concentrated under reduced pressure. The residue was purified by silica gel column chromatography, eluting with PE/EtOAc (4:1) to afford the product (0.2 g, 50%, 0.4 mmol), LC-MS (ESI): m/z [M+H]⁺=489.5.

### Step 3: cis-3-(3-((1,3-bis(4-methoxybenzyl)-2,2-dioxido-1,3-dihydrobenzo[c][1.2.5]thiadiazol-5-yl)amino)-1H-pyrazol-5-yl)cyclopentyl isopropylcarbamate

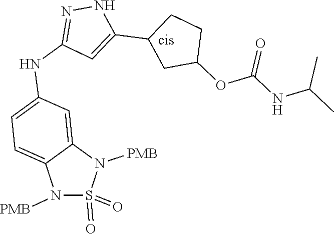

A mixture of 5-bromo-1,3-bis(4-methoxybenzyl)-1,3-dihydrobenzo[c][1,2,5]thiadiazole 2,2-dioxide (0.2 g, 0.4 mmol), cis-3-(3-amino-1H-pyrazol-5-yl)cyclopentyl isopropylcarbamate (0.1 g, 0.4 mmol), Brettephos Pd G3 (36 mg, 0.04 mmol) and K₂CO₃ (165 mg, 1.2 mmol) in t-BuOH (15 mL) was stirred for 16 h at 110° C. in the sealed tube under N₂ atmosphere. The solution was concentrated under reduced pressure. The residue was purified by silica gel column chromatography, eluting with PE/EtOAc (1:4) to afford the crude product (90 mg, 34%), LC-MS (ESI): m/z [M+H]⁺=661.4.

### Step 4: cis-3-(3-((2,2-dioxido-1,3-dihydrobenzo[c][1.2.5]thiadiazol-5-yl)amino)-1H-pyrazol-5-yl)cyclopentyl isopropylcarbamate

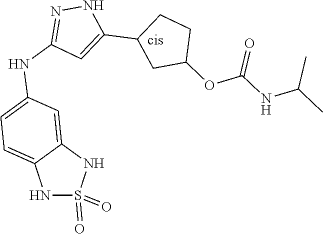 [SMILES: CC(C)NC(=O)OC1CCC(c2cc(Nc3ccc4c(c3)NS(=O)(=O)N4)n[nH]2)C1]

To a stirred solution of cis-3-(3-((1,3-bis(4-methoxybenzyl)-2,2-dioxido-1,3-dihydrobenzo[c][1,2,5]thiadiazol-5-yl)amino)-1H-pyrazol-5-yl)cyclopentyl isopropylcarbamate (60 mg, 0.09 mmol) in DCM (5 mL) was added triflic acid (2 mL). The solution was stirred at room temperature for 3 h. The solution was diluted with DCM (10 mL) and quenched with MeOH. The solution was concentrated under reduced pressure. The residue was purified by prep HPLC (Waters SunFire C18: RD-CO-095 column, eluting with 20%-60% of acetonitrile (containing 0.1% FA) in water (containing 0.1% FA) to afford the product (10 mg, 26%). ¹H NMR (500 MHz, DMSO-d₆) δ 10.80 (s, 1H), 10.44 (s, 1H), 6.95-6.82 (m, 2H), 6.75-6.67 (m, 2H), 5.68 (s, 1H), 5.07-4.92 (m, 1H), 3.62-3.42 (m, 1H), 3.15-2.95 (m, 1H), 2.07-1.96 (m, 1H), 1.95-1.85 (m, 2H), 1.80-1.55 (m, 4H), 1.05-0.96 (m, 6H). LC-MS (ESI): m/z [M+H]⁺=421.38.

### Example 321: cis-3-(3-((3-methyl-1,1-dioxido-2,3-dihydrobenzo[d]isothiazol-5-yl)amino)-1H-pyrazol-5-yl)cyclopentyl isopropylcarbamate

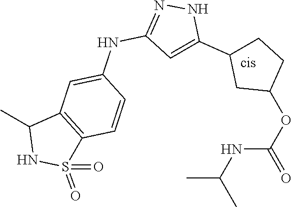 [SMILES: CC(C)NC(=O)OC1CCC(c2cc(Nc3ccc4c(c3)C(C)NS4(=O)=O)n[nH]2)C1]

The titled compound was synthesized in the procedures similar to Example 51 in racemic form, which was further separated by Chiral Prep-HPLC to give:

Enantiomer 1 (Example 321a, 100% ee); Retention time: 11.76 min. ¹H NMR (500 MHz, DMSO-d₆) δ 11.92 (s, 1H), 9.01 (s, 1H), 7.57 (d, J=4.6 Hz, 1H), 7.53-7.46 (m, 2H), 7.32 (d, J=8.5 Hz, 1H), 6.94 (d, J=7.1 Hz, 1H), 5.69 (s, 1H), 5.08-4.92 (m, 1H), 4.65-4.53 (m, 1H), 3.66-3.48 (m, 1H), 3.13-2.99 (m, 1H), 2.48-2.42 (m, 1H), 2.07-1.97 (m, 1H), 1.96-1.85 (m, 1H), 1.79-1.66 (m, 2H), 1.65-1.55 (m, 1H), 1.39 (d, J=6.7 Hz, 3H), 1.03 (d, J=6.2 Hz, 6H) . . . . LC-MS (ESI): m/z [M+H]⁺=434.30.

Enantiomer 2 (Example 321b, 93.76% ee); Retention time: 13.20 min. ¹H NMR (500 MHz, DMSO-d₆) δ 11.92 (s, 1H), 9.01 (s, 1H), 7.57 (d, J=4.6 Hz, 1H), 7.55-7.45 (m, 2H), 7.32 (d, J=8.4 Hz, 1H), 6.94 (d, J=7.0 Hz, 1H), 5.69 (s, 1H), 5.08-4.95 (m, 1H), 4.64-4.54 (m, 1H), 3.63-3.52 (m, 1H), 3.13-2.97 (m, 1H), 2.48-2.41 (m, 1H), 2.07-1.96 (m, 1H), 1.95-1.85 (m, 1H), 1.79-1.67 (m 2H), 1.66-1.55 (m, 1H), 1.39 (d, J=6.7 Hz, 3H), 1.09-0.97 (m, 6H). LC-MS (ESI): m/z [M+H]⁺=434.30.

Enantiomer 3 (Example 321c, 100% ee); Retention time: 6.99 min. ¹H NMR (500 MHz, DMSO-d₆) δ 11.92 (s, 1H), 9.01 (s, 1H), 7.57 (d, J=4.6 Hz, 1H), 7.54-7.44 (m, 2H), 7.32 (d, J=8.3 Hz, 1H), 6.94 (d, J=7.5 Hz, 1H), 5.69 (s, 1H), 5.08-4.93 (m, 1H), 4.67-4.54 (m, 1H), 3.65-3.50 (m, 1H), 3.15-2.97 (m, 1H), 2.48-2.41 (m, 1H), 2.08-1.96 (m, 1H), 1.96-1.84 (m, 1H), 1.80-1.66 (m, 2H), 1.65-1.55 (m, 1H), 1.39 (d, J=6.7 Hz, 3H), 1.09-0.95 (m, 6H). LC-MS (ESI): m/z [M+H]⁺=434.30.

Enantiomer 4 (Example 321d, 93.92% ee); Retention time: 8.19 min. ¹H NMR (500 MHz, DMSO-d₆) δ 1 11.92 (s, 1H), 9.01 (s, 1H), 7.57 (d, J=4.6 Hz, 1H), 7.55-7.45 (m, 2H), 7.32 (d, J=8.1 Hz, 1H), 6.94 (d, J=7.3 Hz, 1H), 5.69 (s, 1H), 5.07-4.93 (m, 1H), 4.66-4.54 (m, 1H), 3.65-3.50 (m, 1H), 3.15-2.97 (m, 1H), 2.48-2.42 (m, 1H), 2.08-1.97 (m, 1H), 1.97-1.85 (m, 1H), 1.79-1.66 (m, 2H), 1.65-1.54 (m, 1H), 1.39 (d, J=6.7 Hz, 3H), 1.03 (d, J=6.0 Hz, 6H). LC-MS (ESI): m/z [M+H]⁺=434.30.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 322: cis-3-(3-((3-methyl-1,1-dioxido-2,3-dihydrobenzo[d]isothiazol-5-yl)amino)-1H-pyrazol-5-yl)cyclopentyl(1-methylcyclopropyl)carbamate

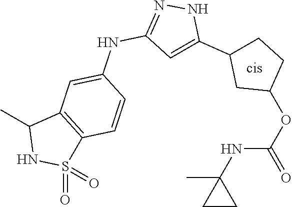 [SMILES: CC1NS(=O)(=O)c2ccc(Nc3cc(C4CCC(OC(=O)NC5(C)CC5)C4)[nH]n3)cc21]

A solution of cis-3-(5-((3-methyl-1,1-dioxido-2,3-dihydrobenzo[d]isothiazol-5-yl)amino)-1H-pyrazol-3-yl)cyclopentyl(4-nitrophenyl) carbonate (200 mg, 0.38 mmol), 1-methylcyclopropan-1-amine hydrochloride (82 mg, 0.76 mmol) and DIEA (294 mg, 2.28 mmol) in THF (10 mL) was stirred for 48 h at 50° C. The solution was concentrated under reduced pressure. The residue was purified by prep HPLC (Waters SunFire C18: RD-CO-095 column, eluting with 10%-60% of acetonitrile (containing 0.1% FA) in water (containing 0.1% FA) to afford the product (55 mg) in a racemic form, which was further separated by Chiral Prep-HPLC to give:

Enantiomer 1 (Example 322a, 100% ee); Retention time: 10.79 min. ¹H NMR (500 MHz, DMSO-d₆) δ 11.85 (s, 1H), 8.94 (s, 1H), 7.50 (d, J=3.8 Hz, 1H), 7.43 (d, J=8.6 Hz, 2H), 7.32-7.15 (m, 2H), 5.62 (s, 1H), 5.07-4.82 (m, 1H), 4.55-4.48 (m, 1H), 3.05-2.92 (m, 1H), 2.39-2.33 (m, 1H), 2.05-1.91 (m, 1H), 1.88-1.78 (m, 1H), 1.75-1.57 (m, 2H), 1.54-1.46 (m, 1H), 1.32 (d, J=6.7 Hz, 3H), 1.16 (s, 3H), 0.58-0.47 (m, 2H), 0.42-0.36 (m, 2H). LC-MS (ESI): m/z [M+H]⁺=446.40.

Enantiomer 2 (Example 322b, 94.94% ee); Retention time: 12.41 min. ¹H NMR (500 MHz, DMSO-d₆) δ 11.99 (brs, 1H), 9.07 (s, 1H), 7.63 (s, 1H), 7.56 (d, J=8.7 Hz, 2H), 7.45-7.29 (m, 2H), 5.75 (s, 1H), 5.15-4.98 (m, 1H), 4.70-4.60 (m, 1H), 3.18-3.06 (m, 1H), 2.53-2.48 (m, 1H), 2.15-2.06 (m, 1H), 2.01-1.91 (m, 1H), 1.85-1.70 (m, 2H), 1.68-1.60 (m, 1H), 1.46 (d, J=6.6 Hz, 3H), 1.30 (s, 3H), 0.70-0.62 (m, 2H), 0.56-0.47 (m, 2H). LC-MS (ESI): m/z [M+H]⁺=446.40.

Enantiomer 3 (Example 322c, 100% ee); Retention time: 8.33 min. ¹H NMR (500 MHz, DMSO-d₆) δ 11.92 (s, 1H), 9.01 (s, 1H), 7.63-7.53 (m, 1H), 7.52-7.42 (m, 2H), 7.39-7.19 (m, 2H), 5.69 (s, 1H), 5.10-4.90 (m, 1H), 4.65-4.50 (m, 1H), 3.12-2.97 (m, 1H), 2.30-2.20 (m, 1H), 2.06-1.95 (m, 1H), 1.94-1.85 (m, 1H), 1.75-1.52 (m, 3H), 1.40 (d, J=6.7 Hz, 3H), 1.24 (s, 3H), 0.63-0.53 (m, 2H), 0.50-0.41 (m, 2H). LC-MS (ESI): m/z [M+H]⁺=446.30.

Enantiomer 4 (Example 322d, 98.34% ee); Retention time: 10.23 min. ¹H NMR (500 MHz, DMSO-d₆) δ 11.92 (brs, 1H), 9.01 (s, 1H), 7.57 (d, J=3.9 Hz, 1H), 7.54-7.43 (m, 2H), 7.40-7.20 (m, 2H), 5.69 (s, 1H), 5.10-4.90 (m, 1H), 4.69-4.51 (m, 1H), 3.13-2.99 (m, 1H), 2.47-2.42 (m, 1H), 2.08-1.95 (m, 1H), 1.94-1.84 (m, 1H), 1.78-1.52 (m, 3H), 1.39 (d, J=6.7 Hz, 3H), 1.24 (s, 3H), 0.64-0.54 (m, 2H), 0.50-0.42 (m, 2H). LC-MS (ESI): m/z [M+H]⁺=446.3.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=30: 70 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=30: 70 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 323: cis-3-(3-((2,2-dioxido-1,3-dihydrobenzo[c]isothiazol-5-yl)amino)-1H-pyrazol-5-yl)cyclopentyl((S)-sec-butyl)carbamate

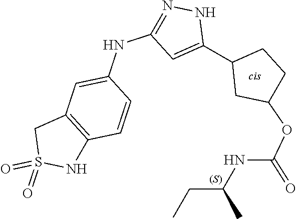

The titled compound was synthesized in the procedures similar to Example 195 in racemic form, which was further separated by Chiral Prep-HPLC to give:

Enantiomer 1 (Example 323a, 100% ee); Retention time: 4.75 min. ¹H NMR (500 MHz, DMSO-d₆) δ 11.61 (s, 1H), 9.73 (s, 1H), 8.17 (s, 1H), 7.38 (s, 1H), 7.16 (d, J=7.4 Hz, 1H), 6.87 (d, J=8.1 Hz, 1H), 6.71 (d, J=8.5 Hz, 1H), 5.57 (s, 1H), 4.99 (s, 1H), 4.43 (s, 2H), 3.41-3.36 (m, 1H), 3.08-2.96 (m, 1H), 2.47-2.42 (m, 1H), 2.03-1.85 (m, 2H), 1.75-1.53 (m, 3H), 1.41-1.31 (m, 2H), 1.01 (d, J=6.4 Hz, 3H), 0.80 (t, J=7.4 Hz, 3H). LC-MS (ESI): m/z [M+H]⁺=434.2.

Enantiomer 2 (Example 323b, 100% ee); Retention time: 7.946 min. ¹H NMR (500 MHz, DMSO-d₆) δ 11.61 (s, 1H), 9.73 (s, 1H), 8.18 (s, 1H), 7.38 (s, 1H), 7.15 (s, 1H), 6.87 (d, J=7.7 Hz, 1H), 6.71 (d, J=8.7 Hz, 1H), 5.57 (s, 1H), 4.98 (s, 1H), 4.43 (s, 2H), 3.41-3.36 (m, 1H), 3.08-2.96 (m, 1H), 2.47-2.42 (m, 1H), 2.03-1.85 (m, 2H), 1.75-1.53 (m, 3H), 1.41-1.31 (m, 2H), 1.01 (d, J=6.4 Hz, 3H), 0.80 (t, J=7.4 Hz, 3H). LC-MS (ESI): m/z [M+H]⁺=434.2.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.2% 2M NH₃ MeOH); Gradient: Mobile Phase A: Mobile Phase B=20: 80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 324: cis-3-(3-((1-methyl-2,2-dioxido-1,3-dihydrobenzo[c]isothiazol-5-yl)amino)-1H-pyrazol-5-yl)cyclopentyl((S)-1-hydroxypropan-2-yl)carbamate

### Step 1: cis-3-(3-((1-methyl-2,2-dioxido-1,3-dihydrobenzo[c]isothiazol-5-yl)amino)-1H-pyrazol-5-yl)cyclopentyl((S)-1-hydroxypropan-2-yl)carbamate

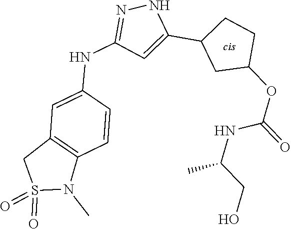 [SMILES: C[C@@H](CO)NC(=O)OC1CCC(c2cc(Nc3ccc4c(c3)CS(=O)(=O)N4C)n[nH]2)C1]

A mixture of cis-4-nitrophenyl 3-((1-methyl-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate (50 mg, 0.074 mmol), DIEA (95.5 mg, 0.74 mmol) and(S)-2-aminopropan-1-ol (27.75 mg, 0.37 mmol) in THF (5 mL) was stirred at 50° C. for 12 h. The mixture was concentrated under reduced pressure. The residue was purified by silica gel column chromatography, eluting with EA to afford the product 50 mg. The residue 50 mg was purified by prep HPLC (Waters XSelect C18: RD-CO-094 column, eluting with a gradient of acetonitrile/water containing 0.1% FA, 20%-40%) to afford the product (20.0 mg, 60.2%). ¹H NMR (500 MHz, DMSO-d₆) δ 11.63 (s, 1H), 8.21 (s, 1H), 7.41 (s, 1H), 7.25 (d, J=7.2 Hz, 1H), 6.79 (t, J=8.3 Hz, 2H), 5.58 (s, 1H), 4.99 (s, 1H), 4.62 (dd, J=11.4, 5.7 Hz, 1H), 4.56 (s, 2H), 3.57-3.42 (m, 1H), 3.23-3.14 (m, 1H), 3.09-2.98 (m, 1H), 2.48-2.41 (m, 1H), 2.48-2.41 (m, 1H), 2.06-1.97 (m, 1H), 1.96-1.82 (m, 1H), 1.80-1.66 (m, 2H), 1.64-1.52 (m, 1H), 1.06-0.94 (m, 3H). LC-MS (ESI): m/z [M+H]⁺=450.2.

### Example 325: cis-3-(3-((1-methyl-2,2-dioxido-1,3-dihydrobenzo[c]isothiazol-5-yl)amino)-1H-pyrazol-5-yl)cyclopentyl(1-hydroxy-2-methylpropan-2-yl)carbamate

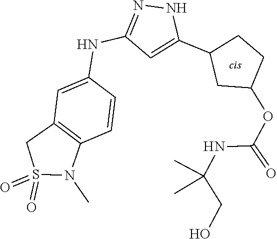 [SMILES: CN1c2ccc(Nc3cc(C4CCC(OC(=O)NC(C)(C)CO)C4)[nH]n3)cc2CS1(=O)=O]

The titled compound was synthesized in the procedures similar to Example 324 in a racemic form, which was further separated by Chiral Prep-HPLC to give:

Enantiomer 1 (Example 325a, 100% ee); Retention time: 5.772 min. ¹H NMR (500 MHz, DMSO-d₆) δ 11.62 (s, 1H), 8.22 (s, 1H), 7.41 (s, 1H), 7.25 (d, J=7.4 Hz, 1H), 6.78 (d, J=8.7 Hz, 1H), 6.47 (s, 1H), 5.58 (s, 1H), 4.96 (s, 1H), 4.70 (t, J=5.9 Hz, 1H), 4.56 (s, 2H), 3.05-2.97 (m, 1H), 2.95 (s, 3H), 2.49-2.38 (m, 1H), 2.06-1.96 (m, 1H), 1.96-1.85 (m, 1H), 1.81-1.66 (m, 3H), 1.65-1.53 (m, 1H), 1.14 (s, 6H). LC-MS (ESI): m/z [M+H]⁺=464.2.

Enantiomer 2 (Example 325b, 100% ee); Retention time: 7.165 min. ¹H NMR (500 MHz, DMSO-d₆) δ 11.62 (s, 1H), 8.22 (s, 1H), 7.41 (s, 1H), 7.25 (d, J=7.4 Hz, 1H), 6.78 (d, J=8.7 Hz, 1H), 6.47 (s, 1H), 5.58 (s, 1H), 4.96 (s, 1H), 4.70 (t, J=5.9 Hz, 1H), 4.56 (s, 2H), 3.05-2.97 (m, 1H), 2.95 (s, 3H), 2.49-2.38 (m, 1H), 2.06-1.96 (m, 1H), 1.96-1.85 (m, 1H), 1.81-1.66 (m, 3H), 1.65-1.53 (m, 1H), 1.14 (s, 6H). LC-MS (ESI): m/z [M+H]⁺=464.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=50: 50 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 326: cis-3-(3-((4-fluoro-2-((1-(methylsulfonyl) azetidin-3-yl)methyl)-1,1-dioxido-2,3-dihydrobenzo[d]isothiazol-5-yl)amino)-1H-pyrazol-5-yl)cyclopentyl isopropylcarbamate

### Step 1:5-bromo-4-fluoro-2-((1-(methylsulfonyl) azetidin-3-yl)methyl)-2,3-dihydrobenzo[d]isothiazole 1,1-dioxide

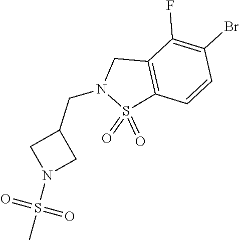 [SMILES: CS(=O)(=O)N1CC(CN2Cc3c(ccc(Br)c3F)S2(=O)=O)C1]

To a stirring solution of 5-bromo-4-fluoro-2,3-dihydrobenzo[d]isothiazole 1,1-dioxide (200 mg, 0.71 mmol) in toluene (10 mL) and (1-(methylsulfonyl) azetidin-3-yl) methanol (234.3 mg, 1.42 mmol) was added 2-(Tributylphosphoranylidene) acetonitrile (513.3 mg, 2.13 mmol) dropwise. The solution was stirred at 90° C. for 16 h under N₂ atmosphere. The mixture was concentrated under reduced pressure. The residue was purified by silica gel column chromatography, eluting with PE/EtOAc (2:1) to afford the product (150 mg, 49.3%), LC-MS (ESI): m/z [M+H]⁺=412.9.

### Step 2: cis-3-(3-((4-fluoro-2-((1-(methylsulfonyl) azetidin-3-yl)methyl)-1,1-dioxido-2,3-dihydrobenzo[d]isothiazol-5-yl)amino-1H-pyrazol-5-yl)cyclopentyl isopropylcarbamate

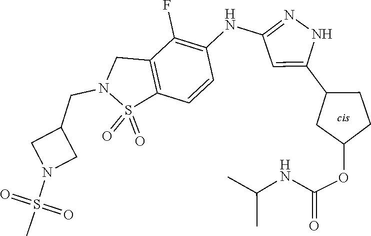 [SMILES: CC(C)NC(=O)OC1CCC(c2cc(Nc3ccc4c(c3F)CN(CC3CN(S(C)(=O)=O)C3)S4(=O)=O)n[nH]2)C1]

A mixture of 5-bromo-4-fluoro-2-((1-(methylsulfonyl) azetidin-3-yl)methyl)-2,3-dihydrobenzo[d]isothiazole 1,1-dioxide (100 mg, 0.24 mmol), cis-3-(3-amino-1H-pyrazol-5-yl)cyclopentyl isopropylcarbamate (59.1 mg, 0.24 mmol), Brettphos G3 Pd (21.6 mg, 0.024 mmol) and K₂CO₃ (99.36 mg, 0.72 mmol) in t-BuOH (20 mL) was stirred at 110° C. for 16 h under N₂ atmosphere. The mixture was concentrated under reduced pressure. The residue was purified by silica gel column chromatography, eluting with EA to afford the product 150 mg. The residue 50 mg was purified by prep HPLC (Waters XSelect C18: RD-CO-094 column, eluting with a gradient of acetonitrile/water containing 0.1% FA, 20%-40%) to afford the product (35.7 mg, 37.2%). ¹H NMR (500 MHz, DMSO-d₆) δ 12.01 (s, 1H), 8.80 (s, 1H), 8.30 (s, 1H), 7.56 (d, J=8.7 Hz, 1H), 6.94 (d, J=7.3 Hz, 1H), 5.83 (s, 1H), 5.00 (s, 1H), 4.52 (s, 2H), 3.98 (t, J=8.2 Hz, 2H), 3.74-3.66 (m, 2H), 3.62-3.52 (m, 1H), 3.41 (d, J=7.4 Hz, 2H), 3.11-3.05 (m, 1H), 2.99-2.91 (m, 1H), 2.47-2.39 (m, 1H), 2.48-2.41 (m, 1H), 2.06-1.97 (m, 1H), 1.96-1.82 (m, 1H), 1.80-1.66 (m, 2H), 1.64-1.52 (m, 1H), 1.11-0.95 (m, 6H). LC-MS (ESI): m/z [M+H]⁺=585.2.

### Example 327: cis-3-(3-((4-fluoro-1-methyl-2,2-dioxido-1,3-dihydrobenzo[c]isothiazol-5-yl)amino)-1H-pyrazol-5-yl)cyclopentyl(1-hydroxy-2-methylpropan-2-yl)carbamate

### Step 1: cis-4-nitrophenyl 3-((4-fluoro-1-methyl-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate

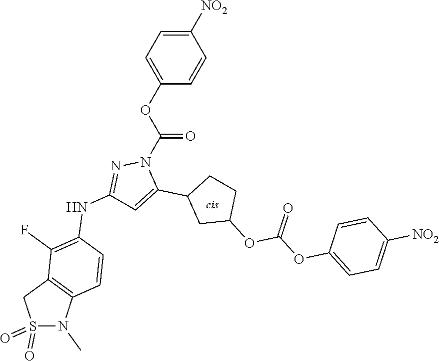 [SMILES: CN1c2ccc(Nc3cc(C4CCC(OC(=O)Oc5ccc([N+](=O)[O-])cc5)C4)n(C(=O)Oc4ccc([N+](=O)[O-])cc4)n3)c(F)c2CS1(=O)=O]

The titled compound was synthesized in the procedures similar to Example 196, step 1-4. LC-MS (ESI): m/z [M+H]⁺=697.2.

### Step 2: cis-3-(3-((4-fluoro-1-methyl-2,2-dioxido-1,3-dihydrobenzo[c]isothiazol-5-yl)amino)-1H-pyrazol-5-yl)cyclopentyl(1-hydroxy-2-methylpropan-2-yl)carbamate

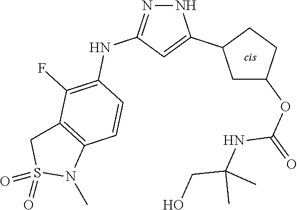 [SMILES: CN1c2ccc(Nc3cc(C4CCC(OC(=O)NC(C)(C)CO)C4)[nH]n3)c(F)c2CS1(=O)=O]

A mixture of cis-4-nitrophenyl 3-((4-fluoro-1-methyl-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate (50 mg, 0.074 mmol), DIEA (95.5 mg, 0.74 mmol) and 2-amino-2-methylpropan-1-ol (33 mg, 0.37 mmol) in THF (5 mL) was stirred at 50° C. for 12 h. The mixture was concentrated under reduced pressure. The residue was purified by silica gel column chromatography, eluting with EA to afford the product in a racemic form, which was further separated with Chiral Prep-HPLC to give:

Enantiomer 1 (Example 327a, 100% ee); Retention time: 5.479 min. ¹H NMR (500 MHz, DMSO-d₆) δ 11.69 (s, 1H), 8.04 (t, J=8.8 Hz, 1H), 7.91 (s, 1H), 6.68 (d, J=8.8 Hz, 1H), 6.45 (s, 1H), 5.68 (s, 1H), 4.96 (s, 1H), 4.72 (s, 2H), 4.69 (t, J=5.9 Hz, 1H), 3.33 (s, 2H), 3.05-2.98 (m, 4H), 2.47-2.42 (m, 1H), 2.05-1.83 (m, 2H), 1.75-1.53 (m, 3H), 1.14 (s, 6H). LC-MS (ESI): m/z [M+H]⁺=482.3.

Enantiomer 2 (Example 327b, 100% ee); Retention time: 7.382 min. ¹H NMR (500 MHz, DMSO-d₆) δ 11.69 (s, 1H), 8.04 (s, 1H), 7.92 (s, 1H), 6.68 (d, J=8.8 Hz, 1H), 6.46 (s, 1H), 5.68 (s, 1H), 4.97 (s, 1H), 4.72 (s, 2H), 4.69 (t, J=5.9 Hz, 1H), 3.33 (s, 2H), 3.05-2.96 (m, 4H), 2.47-2.42 (m, 1H), 2.03-1.84 (m, 2H), 1.75-1.53 (m, 3H), 1.14 (s, 6H). LC-MS (ESI): m/z [M+H]⁺=482.3.

Chiral analytical method: Column: CHIRALPAK IE 4.6 mm×250 mm, 5 μm; Mobile phase: A for MTBE (0.1% 2M NH₃ MeOH) and B for MEOH/DCM (50/50); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MEOH/DCM (50/50); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 328: cis-3-(3-((4-fluoro-1,1-dioxido-2,3-dihydrobenzo[d]isothiazol-5-yl)amino)-1H-pyrazol-5-yl)cyclopentyl((S)-1-hydroxypropan-2-yl)carbamate

### Step 1:5-bromo-4-fluoro-2-(4-methoxybenzyl)-2,3-dihydrobenzo[d]isothiazole 1,1-dioxide

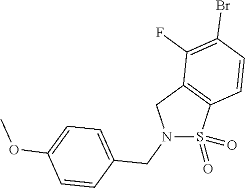 [SMILES: COc1ccc(CN2Cc3c(ccc(Br)c3F)S2(=O)=O)cc1]

To a solution of 5-bromo-4-fluoro-2,3-dihydrobenzo[d]isothiazole 1,1-dioxide (13 g, 50 mmol) in 100 mL DMF was added K₂CO₃ (13.8 g, 100 mL) and 1-(chloromethyl)-4-methoxybenzene (8.6 g, 55 mmol). The resulting mixture was stirred at room temperature for overnight. The mixture was poured into water and filtrated. The filtrate was washed by water and dried to obtain the product (15.3 g, 85% yield).

LC-MS (ESI): m/z [M+H]⁺=385.9.

### Step 2: cis-3-(5-amino-1-(tert-butyl)-1H-pyrazol-3-yl)cyclopentan-1-ol

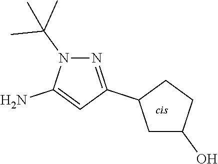 [SMILES: CC(C)(C)n1nc(C2CCC(O)C2)cc1N]

To a solution of cis-benzyl(1-(tert-butyl)-3-(3-hydroxycyclopentyl)-1H-pyrazol-5-yl)carbamate (7.1 g, 20 mmol) in MeOH (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (200 mL×3). The filtrate was concentrated under reduced pressure to afford the product (4.2 g, 94% yield). LC-MS (ESI): m/z [M+H]⁺=224.2.

### Step 3: cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide

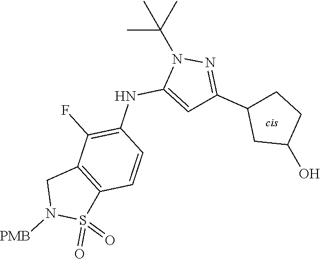

To a solution of 5-bromo-4-fluoro-2-(4-methoxybenzyl)-2,3-dihydrobenzo[d]isothiazole 1,1-dioxide (8.0 g, 20.7 mmol) and cis-3-(5-amino-1-(tert-butyl)-1H-pyrazol-3-yl)cyclopentan-1-ol (4.62 g, 20.7 mmol) in t-BuOH (80 mmol) was added BrettPhos Pd G3 (940 mg, 1.035 mmol) and K₂CO₃ (8.6 g, 62.1 mmol), the reaction mixture was stirred for 16 hours at 100° C. under nitrogen. The mixture was cooled to room temperature, filtered, and the filtrate was concentrated under reduced pressure. The residue was purified by silica gel column chromatography, eluting with PE/EA (1:2) to afford the product (9.33 g, 85%). LC-MS (ESI): m/z [M+H]⁺=529.1.

### Step 4: cis-3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl(4-nitrophenyl) carbonate

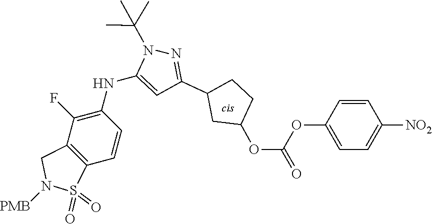

To a solution of cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (9.33 g, 17.67 mmol), pyridine (4.2 g. 53.01 mmol) and DMAP (215 mg, 1.167 mmol) in dichloromethane (100 mL) was added 4-nitrophenyl carbonochloridate (4.3 g, 21.2 mmol), the reaction mixture was stirred for 2 hours at room temperature. The resulting mixture was concentrated under reduce pressure, the residue was purified by silica gel column chromatography, eluting with PE/EA (3:1) to afford the product (10.0 g, 82%). LC-MS (ESI): m/z [M+H]⁺=694.6.

### Step 5: cis-3-(3-((4-fluoro-1,1-dioxido-2,3-dihydrobenzo[d]isothiazol-5-yl)amino)-1H-pyrazol-5-yl)cyclopentyl(4-nitrophenyl) carbonate

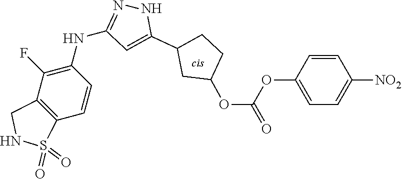 [SMILES: O=C(Oc1ccc([N+](=O)[O-])cc1)OC1CCC(c2cc(Nc3ccc4c(c3F)CNS4(=O)=O)n[nH]2)C1]

cis-3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl(4-nitrophenyl) carbonate (5.0 g, 7.2 mmol) was dissolved in formic acid (50 mL), The reaction mixture was stirred for 16 hours at 90° C. The resulting mixture was cooled to room temperature, concentrated under reduce pressure, dried over in vacuum, to afford crude product without further purification. (4.93 g crude). LC-MS (ESI): m/z [M+H]⁺=518.3.

### Step 6: cis-3-(3-((4-fluoro-1,1-dioxido-2,3-dihydrobenzo[d]isothiazol-5-yl)amino)-1H-pyrazol-5-yl)cyclopentyl((S)-1-hydroxypropan-2-yl)carbamate

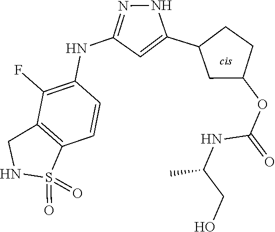 [SMILES: C[C@@H](CO)NC(=O)OC1CCC(c2cc(Nc3ccc4c(c3F)CNS4(=O)=O)n[nH]2)C1]

A mixture of cis-3-(3-((4-fluoro-1,1-dioxido-2,3-dihydrobenzo[d]isothiazol-5-yl)amino)-1H-pyrazol-5-yl)cyclopentyl(4-nitrophenyl) carbonate (50 mg, 0.97 mmol), DIEA (258 mg, 2 mmol) and (S)-2-aminopropan-1-ol (150 mg, 2 mmol) in THF (5 mL) was stirred at 50° C. for 12 h. The mixture was concentrated under reduced pressure. The residue was purified by silica gel column chromatography, eluting with PE and EA to afford the product in a racemic form, which was further separated by Chiral Prep-HPLC to give:

Enantiomer 1 (Example 328a, 100% ee); Retention time: 6.977 min. ¹H NMR (500 MHz, DMSO-d₆) δ=11.98 (s, 1H), 8.71 (s, 1H), 8.26 (t, J=7.7, 1H), 7.69 (s, 1H), 7.48 (d, J=8.6, 1H), 6.79 (d, J=7.9, 1H), 5.83 (s, 1H), 5.00 (m, 1H), 4.62 (t, J=5.3, 1H), 4.41 (d, J=2.9, 2H), 3.51-3.45 (m, 1H), 3.22-3.14 (m, 1H), 3.07 (m, 1H), 2.46 (m, 2H), 2.06-1.97 (m, 1H), 1.97-1.85 (m, 1H), 1.71 (m, 2H), 1.60 (m, 1H), 1.00 (d, J=6.6, 3H). LC-MS (ESI): m/z [M+H]⁺=454.31.

Enantiomer 2 (Example 328b, 100% ee); Retention time: 4.261 min. ¹H NMR (500 MHz, DMSO-d₆) δ=11.98 (s, 1H), 8.71 (s, 1H), 8.26 (s, 1H), 7.69 (s, 1H), 7.48 (d, J=8.5, 1H), 6.80 (d, J=7.8, 1H), 5.83 (s, 1H), 5.00 (m, 1H), 4.62 (t, J=5.4, 1H), 4.41 (d, J=4.6, 2H), 3.54-3.44 (m, 1H), 3.21-3.13 (m, 1H), 3.13-2.99 (m, 1H), 2.49-2.42 (m, 2H), 2.06-1.98 (m, 1H), 1.91 (m, 1H), 1.71 (m, 2H), 1.60 (m, 1H), 1.00 (d, J=6.4, 3H). LC-MS (ESI): m/z [M+H]⁺=454.29.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=20: 80 (v/v); HPLC Equipment: HPLC-Agilent; Column temperature: 35° C.

Prep-HPLC Condition: Sunfire Prep C18 OBD™ 5 μm, 19*150 mm 5 μm, eluting with 23%-35% of water (containing 0.1% FA) in acetonitrile (containing 0.1% FA); Flow Rate: 17 mL/min, Wavelength: UV 214 nm and 254 nm, Prep-HPLC Equipment: Prep-HPLC-Gilson; Column temperature: 25° C.

### Example 329: cis-3-(3-((4-fluoro-1,1-dioxido-2,3-dihydrobenzo[d]isothiazol-5-yl)amino)-1H-pyrazol-5-yl)cyclopentyl(1-hydroxy-2-methylpropan-2-yl)carbamate

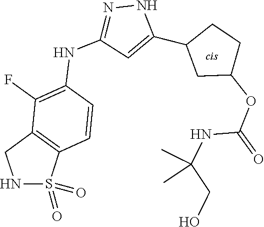 [SMILES: CC(C)(CO)NC(=O)OC1CCC(c2cc(Nc3ccc4c(c3F)CNS4(=O)=O)n[nH]2)C1]

The titled compound was synthesized in the procedures similar to Example 328. ¹H NMR (500 MHz, DMSO-d₆) δ=11.98 (s, 1H), 8.72 (s, 1H), 8.27 (t, J=7.7, 1H), 7.69 (s, 1H), 7.48 (d, J=8.6, 1H), 6.47 (s, 1H), 5.83 (s, 1H), 4.97 (m, 1H), 4.70 (t, J=5.9, 1H), 4.41 (d, J=3.4, 2H), 3.33 (s, 2H), 3.10-3.00 (m, 1H), 2.48-2.44 (m, 1H), 2.05-1.97 (m, 1H), 1.94-1.84 (m, 1H), 1.76-1.64 (m, 2H), 1.59 (m, 1H), 1.14 (s, 6H). LC-MS (ESI): m/z [M+H]⁺=468.30.

### Example 330: cis-3-(3-((4-fluoro-1-methyl-2,2-dioxido-1,3-dihydrobenzo[c]isothiazol-5-yl)amino)-1H-pyrazol-5-yl)cyclopentyl(2-hydroxy-2-methylpropyl)carbamate

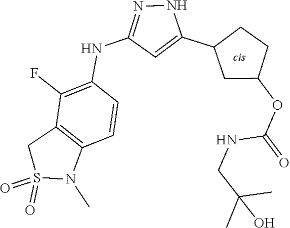 [SMILES: CN1c2ccc(Nc3cc(C4CCC(OC(=O)NCC(C)(C)O)C4)[nH]n3)c(F)c2CS1(=O)=O]

The titled compound was synthesized in the procedures similar to Example 327 in a racemic form, which was separated by Chiral Prep-HPLC to give:

Enantiomer 1 (Example 330a, 100% ee); Retention time: 16.878 min. ¹H NMR (500 MHz, DMSO-d₆) δ 11.71 (s, 1H), 8.03 (s, 1H), 7.91 (s, 1H), 6.85 (t, J=5.9 Hz, 1H), 6.68 (d, J=8.7 Hz, 1H), 5.69 (s, 1H), 5.00 (s, 1H), 4.72 (s, 2H), 4.33 (s, 1H), 3.07-2.97 (m, 4H), 2.92 (d, J=6.2 Hz, 2H), 2.48-2.42 (m, 1H), 2.04-1.85 (m, 2H), 1.78-1.55 (m, 3H), 1.02 (s, 6H). LC-MS (ESI): m/z [M+H]⁺=482.3.

Enantiomer 1 (Example 330b, 97% ee); Retention time: 19.09 min. ¹H NMR (500 MHz, DMSO-d₆) δ 11.71 (s, 1H), 8.03 (s, 1H), 7.91 (s, 1H), 6.85 (t, J=5.9 Hz, 1H), 6.68 (d, J=8.7 Hz, 1H), 5.69 (s, 1H), 5.00 (s, 1H), 4.72 (s, 2H), 4.33 (s, 1H), 3.07-2.97 (m, 4H), 2.92 (d, J=6.2 Hz, 2H), 2.48-2.42 (m, 1H), 2.04-1.85 (m, 2H), 1.78-1.55 (m, 3H), 1.02 (s, 6H). LC-MS (ESI): m/z [M+H]⁺=482.3.

Chiral analytical method: Column: CHIRALPAK IE, 4.6*250 mm 5 μm; Mobile phase: A for MTBE and B for MeOH: DCM=70:30 (0.1% 2M NH₃ MeOH); Gradient: Mobile Phase A: Mobile Phase B=50: 50 (v/v); HPLC Equipment: HPLC-Agilent,Back pressure: 100 bar; Column temperature: 35° C.

Chiral Prep-HPLC Condition: CHIRALPAK IE, 20*250 mm 5 μm, 5 μm; Mobile phase: A for MTBE and B for MeOH: DCM=70:30 (0.2% 2M NH₃ MeOH); Gradient: Mobile Phase A: Mobile Phase B=50: 50 (v/v); Flow Rate: 18 mL/min, Wave Length: UV 270 nm and 310 nm, Prep-HPLC Equipment: Prep-HPLC-Gilson,Back pressure: 100 bar; Column temperature: 25° C.

### Example 331: cis-3-(3-((4-fluoro-1-methyl-2,2-dioxido-1,3-dihydrobenzo[c]isothiazol-5-yl)amino)-1H-pyrazol-5-yl)cyclopentyl(1-methoxypropan-2-yl)carbamate

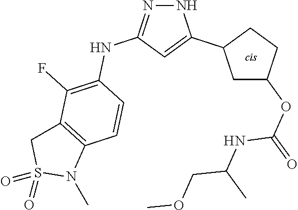 [SMILES: COCC(C)NC(=O)OC1CCC(c2cc(Nc3ccc4c(c3F)CS(=O)(=O)N4C)n[nH]2)C1]

The titled compound was synthesized in the procedures similar to Example 3. ¹H NMR (500 MHz, DMSO-d₆) δ 11.70 (s, 1H), 8.05 (s, 1H), 7.91 (s, 1H), 6.96 (s, 1H), 6.68 (d, J=8.7 Hz, 1H), 5.68 (s, 1H), 5.00 (s, 1H), 4.73 (s, 2H), 3.65 (s, 1H), 3.3-3.22 (m, 4H), 3.2-3.11 (m, 1H), 3.08-2.99 (m, 4H), 2.48-2.41 (m, 1H), 2.08-1.85 (m, 2H), 1.78-1.55 (m, 3H), 1.01 (s, 3H). LC-MS (ESI): m/z [M+H]⁺=482.3.

### Example 332: cis-3-(3-((4-fluoro-1-methyl-2,2-dioxido-1,3-dihydrobenzo[c]isothiazol-5-yl)amino)-1H-pyrazol-5-yl)cyclopentyl(3-methyloxetan-3-yl)carbamate

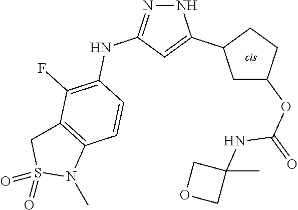 [SMILES: CN1c2ccc(Nc3cc(C4CCC(OC(=O)NC5(C)COC5)C4)[nH]n3)c(F)c2CS1(=O)=O]

The titled compound was synthesized in the procedures similar to Example 327 in a racemic form, which was further separated by Chiral Prep-HPLC to give:

Enantiomer 1 (Example 332a, 98% ee); Retention time: 6.537 min. ¹H NMR (500 MHz, DMSO-d₆) δ 11.72 (s, 1H), 8.04 (t, J=8.2 Hz, 1H), 7.93 (s, 1H), 7.62 (s, 1H), 6.68 (d, J=8.7 Hz, 1H), 5.68 (s, 1H), 5.01 (s, 1H), 4.73 (s, 2H), 4.56 (s, 2H), 4.24 (d, J=4.7 Hz, 2H), 3.04 (dd, J=17.0, 8.3 Hz, 1H), 2.99 (s, 3H), 2.48-2.41 (m, 1H), 2.06-1.96 (m, 1H), 1.90 (s, 1H), 1.71 (dt, J=19.3, 8.1 Hz, 2H), 1.65-1.56 (m, 1H), 1.47 (s, 3H). LC-MS (ESI): m/z [M+H]⁺=480.2.

Enantiomer 2 (Example 332b, 96% ee); Retention time: 7.937 min. ¹H NMR (500 MHz, DMSO-d₆) δ 11.72 (s, 1H), 8.04 (t, J=8.2 Hz, 1H), 7.93 (s, 1H), 7.62 (s, 1H), 6.68 (d, J=8.7 Hz, 1H), 5.68 (s, 1H), 5.01 (s, 1H), 4.73 (s, 2H), 4.56 (s, 2H), 4.24 (d, J=4.7 Hz, 2H), 3.04 (dd, J=17.0, 8.3 Hz, 1H), 2.99 (s, 3H), 2.48-2.41 (m, 1H), 2.06-1.96 (m, 1H), 1.90 (s, 1H), 1.71 (dt, J=19.3, 8.1 Hz, 2H), 1.65-1.56 (m, 1H), 1.47 (s, 3H). LC-MS (ESI): m/z [M+H]⁺=480.2.

Chiral analytical method: Column: CHIRALPAK IE, 4.6*250 mm 5 μm; Mobile phase: A for MTBE and B for MeOH: DCM=50:50 (0.1% 2M NH₃ MeOH); Gradient: Mobile Phase A: Mobile Phase B=50: 50 (v/v); HPLC Equipment: HPLC-Agilent,Back pressure: 100 bar; Column temperature: 35° C.

Chiral Prep-HPLC Condition: CHIRALPAK IE, 20*250 mm 5 μm, 5 μm; Mobile phase: A for MTBE and B for MeOH: DCM=50:50 (0.2% 2M NH₃ MeOH); Gradient: Mobile Phase A: Mobile Phase B=50: 50 (v/v); Flow Rate: 18 mL/min, Wave Length: UV 270 nm and 310 nm, Prep-HPLC Equipment: Prep-HPLC-Gilson,Back pressure: 100 bar; Column temperature: 25° C.

### Example 333: cis-3-(3-((4-fluoro-1,1-dioxido-2,3-dihydrobenzo[d]isothiazol-5-yl)amino)-1H-pyrazol-5-yl)cyclopentyl(1-(hydroxymethyl)cyclopropyl)carbamate

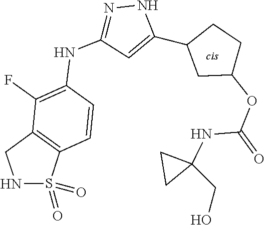 [SMILES: O=C(NC1(CO)CC1)OC1CCC(c2cc(Nc3ccc4c(c3F)CNS4(=O)=O)n[nH]2)C1]

The titled compound was synthesized in the procedures similar to Example 328. ¹H NMR (500 MHz, DMSO-d₆) δ=11.97 (s, 1H), 8.70 (s, 1H), 8.26 (m, 1H), 7.69 (t, J=5.1, 1H), 7.48 (d, J=8.6, 1H), 7.32 (s, 1H), 5.83 (s, 1H), 4.99 (m, 1H), 4.60 (t, J=5.9, 1H), 4.40 (d, J=5.2, 2H), 3.39 (m, 2H), 3.06 (m, 1H), 2.48-2.41 (m, 2H), 2.02 (m, 1H), 1.89 (m, 1H), 1.70 (m, 2H), 1.58 (m, 1H), 0.63 (m, 2H), 0.54 (m, 2H). LC-MS (ESI): m/z [M+H]⁺=466.31.

### Example 334: cis-3-(3-((3,3-difluoro-2,2-dioxido-1,3-dihydrobenzo[c]isothiazol-5-yl)amino)-1H-pyrazol-5-yl)cyclopentyl isopropylcarbamate

### Step 1:5-bromo-3,3-difluoro-1-(4-methoxybenzyl)-1,3-dihydrobenzo[c]isothiazole 2,2-dioxide

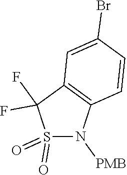

To a mixture of 5-bromo-1-(4-methoxybenzyl)-1,3-dihydrobenzo[c]isothiazole 2,2-dioxide (100 mg, 0.27 mmol) and N-fluoro-N-(phenylsulfonyl)benzenesulfonamide (180 mg, 0.57 mmol) in THF (10 mL) was added LiHMDS (0.65 mL, 0.65 mmol) at −50° C. under a nitrogen atmosphere. The mixture was stirred for 5 h at −50° C. The mixture was quenched by ice water (50 mL), extracted with EA (3×50 mL). The combined organic layers were dried over Na₂SO₄, filtered and concentrated under vacuum. The residue was purified by silica gel column chromatography, eluting with EA/PE (0-60%) to afford the product (41 mg, 37.3%). LC-MS (ESI): m/z [M+H]⁺=404.2.

### Step 2: cis-3-(3-((3,3-difluoro-1-(4-methoxybenzyl)-2,2-dioxido-1,3-dihydrobenzo[c]isothiazol-5-yl)amino)-1H-pyrazol-5-yl)cyclopentyl isopropylcarbamate

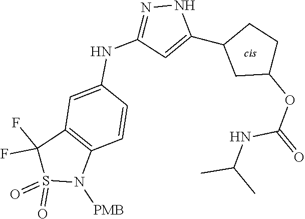

To a mixture of 5-bromo-3,3-difluoro-1-(4-methoxybenzyl)-1,3-dihydrobenzo[c]isothiazole 2,2-dioxide (41 mg, 0.11 mmol), cis-3-(3-amino-1H-pyrazol-5-yl)cyclopentyl isopropylcarbamate (20.5 mg, 0.08 mmol), Brettphos Pd G3 (9.1 mg, 0.01 mmol) and K₂CO₃ (41.4 mg, 0.3 mmol) in t-BuOH (5 mL) was stirred at 110° C. for 16 h under a nitrogen atmosphere. The mixture was concentrated under vacuum. The residue was purified by silica gel column chromatography, eluting with EA/PE (0-100%) to afford the product (25 mg, 42.8%). LC-MS (ESI): m/z [M+H]⁺=576.2.

### Step 3: cis-3-(3-((3,3-difluoro-2,2-dioxido-1,3-dihydrobenzo[c]isothiazol-5-yl)amino)-1H-pyrazol-5-yl)cyclopentyl isopropylcarbamate

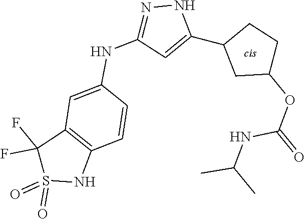 [SMILES: CC(C)NC(=O)OC1CCC(c2cc(Nc3ccc4c(c3)C(F)(F)S(=O)(=O)N4)n[nH]2)C1]

To a solution of cis-3-(3-((3,3-difluoro-1-(4-methoxybenzyl)-2,2-dioxido-1,3-dihydrobenzo[c]isothiazol-5-yl)amino)-1H-pyrazol-5-yl)cyclopentyl isopropylcarbamate (25 mg, 0.043 mmol) in THF (10 mL) and H₂O (2 mL) was added Pd/C (5 mg). The mixture was stirred at rt for 16 h under a hydrogen atmosphere. The mixture was filtered and the filtrate was concentrated under vacuum. The residue was purified by Prep-HPLC to afford the product (10.64 mg, 53.8%). ¹H NMR (500 MHz, DMSO-d₆) δ 11.81 (s, 1H), 10.78 (s, 1H), 8.69 (s, 1H), 7.97 (s, 1H), 7.42 (d, J=10 Hz, 1H), 7.00-6.93 (m, 2H), 5.59 (s, 1H), 5.19-4.97 (m, 1H), 3.65-3.50 (m, 1H), 3.12-2.98 (m, 1H), 2.47-2.41 (m, 1H), 2.05-1.96 (m, 1H), 1.95-1.84 (m, 1H), 1.79-1.65 (m, 2H), 1.63-1.54 (m, 1H), 1.03 (d, J=10 Hz, 6H). LC-MS (ESI): m/z [M+H]⁺=455.2.

### Example 335: cis-3-(3-((4-fluoro-2-methyl-1,1-dioxido-2,3-dihydrobenzo[d]isothiazol-5-yl)amino)-1H-pyrazol-5-yl)cyclopentyl(3-methyltetrahydrofuran-3-yl)carbamate

### Step 1: cis-4-nitrophenyl 3-((2-methyl-4-fluoro-1,1-dioxido-2,3-dihydrobenzo[d]isothiazol-5-yl)amino)-5-(3-(((4-nitrophenoxy) carbonyl)oxy) cyclopentyl)-1H-pyrazole-1-carboxylate

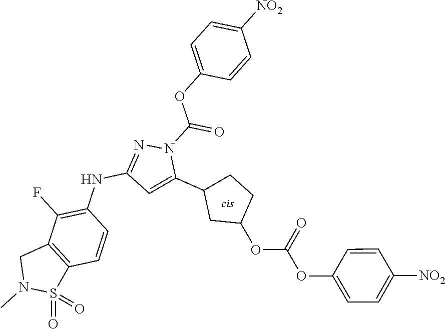 [SMILES: CN1Cc2c(ccc(Nc3cc(C4CCC(OC(=O)Oc5ccc([N+](=O)[O-])cc5)C4)n(C(=O)Oc4ccc([N+](=O)[O-])cc4)n3)c2F)S1(=O)=O]

The titled compound was synthesized in the procedures similar to example 196. LC-MS (ESI): m/z [M+H]⁺=697.2.

### Step 2: cis-3-(3-((4-fluoro-2-methyl-1,1-dioxido-2,3-dihydrobenzo[d]isothiazol-5-yl)amino)-1H-pyrazol-5-yl)cyclopentyl(3-methyltetrahydrofuran-3-yl)carbamate

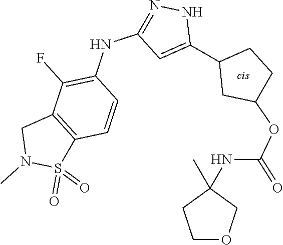 [SMILES: CN1Cc2c(ccc(Nc3cc(C4CCC(OC(=O)NC5(C)CCOC5)C4)[nH]n3)c2F)S1(=O)=O]

To a solution of cis-4-nitrophenyl 3-((2-methyl-4-fluoro-1,1-dioxido-2,3-dihydrobenzo[d]isothiazol-5-yl)amino)-5-(3-(((4-nitrophenoxy) carbonyl)oxy) cyclopentyl)-1H-pyrazole-1-carboxylate (42 mg, 0.06 mmol) in THF (10 mL) was added 3-methyltetrahydrofuran-3-amine (12 mg, 0.12 mmol) and DIEA (31 mg, 0.24 mmol). The result mixture was stirred at 60° C. for 12 h. The solution was concentrated and the residue was purified by silica gel column chromatography, eluting with EA/PE (0-100%) to afford the product (10 mg, 34%). ¹H NMR (500 MHz, DMSO-d₆) δ 12.01 (s, 1H), 8.77 (s, 1H), 8.28 (s, 1H), 7.54 (d, J=8.6 Hz, 1H), 7.23 (s, 1H), 5.84 (s, 1H), 5.00 (s, 1H), 4.43 (s, 2H), 3.86-3.78 (m, 1H), 3.73 (t, J=7.1 Hz, 2H), 3.43 (d, J=8.6 Hz, 1H), 3.12-3.00 (m, 1H), 2.78 (s, 3H), 2.54 (s, 1H), 2.21-2.12 (m, 1H), 2.08-1.97 (m, 1H), 1.92 (dd, J=17.8, 10.6 Hz, 1H), 1.79-1.68 (m, 3H), 1.65-1.54 (m, 1H), 1.32 (s, 3H). LC-MS (ESI): m/z [M+H]⁺=494.2.

### Example 336: cis-3-(3-((4-fluoro-2-methyl-1,1-dioxido-2,3-dihydrobenzo[d]isothiazol-5-yl)amino)-1H-pyrazol-5-yl)cyclopentyl oxetan-3-ylcarbamate

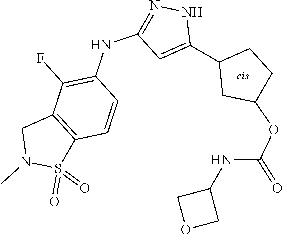 [SMILES: CN1Cc2c(ccc(Nc3cc(C4CCC(OC(=O)NC5COC5)C4)[nH]n3)c2F)S1(=O)=O]

The titled compound was synthesized in the procedures similar to Example 335. ¹H NMR (500 MHz, DMSO-d₆) δ 12.01 (s, 1H), 8.78 (s, 1H), 8.28 (t, J=7.6 Hz, 1H), 7.89 (d, J=5.6 Hz, 1H), 7.54 (d, J=8.6 Hz, 1H), 5.84 (s, 1H), 5.01 (d, J=4.9 Hz, 1H), 4.64 (s, 3H), 4.43 (s, 4H), 3.13-2.97 (m, 1H), 2.78 (s, 3H), 2.45-2.42 (m, 1H), 2.10-1.96 (m, 1H), 1.91 (s, 1H), 1.79-1.67 (m, 2H), 1.62 (s, 1H).

LC-MS (ESI): m/z [M+H]⁺=466.2.

### Example 337: cis-3-(3-((4-fluoro-2-methyl-1,1-dioxido-2,3-dihydrobenzo[d]isothiazol-5-yl)amino)-1H-pyrazol-5-yl)cyclopentyl(1-(oxetan-3-yl)ethyl)carbamate

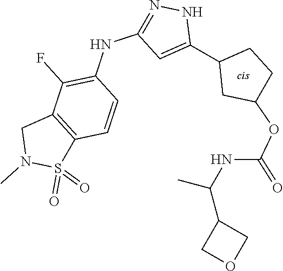 [SMILES: CC(NC(=O)OC1CCC(c2cc(Nc3ccc4c(c3F)CN(C)S4(=O)=O)n[nH]2)C1)C1COC1]

The titled compound was synthesized in the procedures similar to Example 335. ¹H NMR (500 MHz, DMSO-d₆) δ 12.01 (s, 1H), 8.76 (d, J=10.5 Hz, 1H), 8.28 (s, 1H), 7.54 (d, J=8.6 Hz, 1H), 7.18-7.05 (m, 1H), 5.83 (d, J=2.9 Hz, 1H), 5.01 (s, 1H), 4.53 (dt, J=7.8, 6.4 Hz, 2H), 4.43 (s, 2H), 4.34 (t, J=5.3 Hz, 1H), 4.25 (t, J=6.2 Hz, 1H), 3.81 (dd, J=14.8, 7.6 Hz, 1H), 3.08 (d, J=8.0 Hz, 1H), 2.90 (dd, J=13.6, 6.3 Hz, 1H), 2.78 (s, 3H), 2.49-2.40 (m, 2H), 2.05 (t, J=10.5 Hz, 1H), 1.99-1.85 (m, 1H), 1.73 (dd, J=14.5, 8.7 Hz, 2H), 1.61 (dd, J=12.7, 5.7 Hz, 1H), 0.95 (dd, J=6.4, 3.8 Hz, 3H). LC-MS (ESI): m/z [M+H]⁺=494.2.

### Example 338: cis-3-(3-((4-fluoro-2-methyl-1,1-dioxido-2,3-dihydrobenzo[d]isothiazol-5-yl)amino)-1H-pyrazol-5-yl)cyclopentyl(1-methoxypropan-2-yl)carbamate

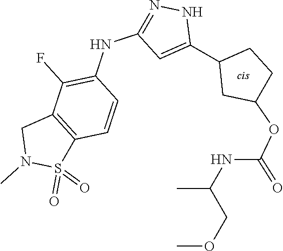 [SMILES: COCC(C)NC(=O)OC1CCC(c2cc(Nc3ccc4c(c3F)CN(C)S4(=O)=O)n[nH]2)C1]

The titled compound was synthesized in the procedures similar to Example 335. ¹H NMR (500 MHz, DMSO-d₆) δ 12.01 (s, 1H), 8.77 (s, 1H), 8.28 (s, 1H), 7.54 (d, J=8.6 Hz, 1H), 6.95 (d, J=8.0 Hz, 1H), 5.84 (s, 1H), 5.01 (s, 1H), 4.43 (s, 2H), 3.70-3.59 (m, 1H), 3.28-3.22 (m, 1H), 3.16-3.10 (m, 1H), 3.09-3.02 (m, 1H), 2.78 (s, 3H), 2.48-2.42 (m, 1H), 2.07-1.99 (m, 1H), 1.97-1.84 (m, 1H), 1.71 (dd, J=19.3, 10.0 Hz, 2H), 1.60 (dd, J=16.3, 8.0 Hz, 1H), 1.06-0.93 (m, 3H). LC-MS (ESI): m/z [M+H]⁺=482.2.

### Example 339: cis-3-(3-((4-fluoro-2-methyl-1,1-dioxido-2,3-dihydrobenzo[d]isothiazol-5-yl)amino)-1H-pyrazol-5-yl)cyclopentyl(1-(hydroxymethyl)cyclopropyl)carbamate

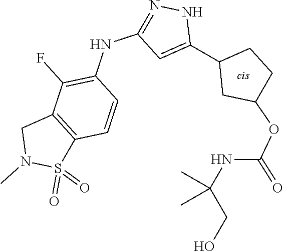 [SMILES: CN1Cc2c(ccc(Nc3cc(C4CCC(OC(=O)NC(C)(C)CO)C4)[nH]n3)c2F)S1(=O)=O]

The titled compound was synthesized in the procedures similar to Example 335. ¹H NMR (500 MHz, DMSO-d₆) δ 12.00 (s, 1H), 8.77 (s, 1H), 8.29 (t, J=7.6 Hz, 1H), 7.54 (d, J=8.6 Hz, 1H), 6.47 (s, 1H), 5.84 (s, 1H), 4.97 (s, 1H), 4.70 (t, J=5.9 Hz, 1H), 4.43 (s, 2H), 3.33 (s, 2H), 3.11-3.00 (m, 1H), 2.78 (s, 3H), 2.48-2.42 (m, 1H), 2.07-1.97 (m, 1H), 1.95-1.83 (m, 1H), 1.78-1.65 (m, 2H), 1.63-1.53 (m, 1H), 1.14 (s, 6H). LC-MS (ESI): m/z [M+H]⁺=482.2.

### Example 340: cis-3-(3-((4-fluoro-2-methyl-1,1-dioxido-2,3-dihydrobenzo[d]isothiazol-5-yl)amino)-1H-pyrazol-5-yl)cyclopentyl(1-hydroxy-2-methylpropan-2-yl)carbamate

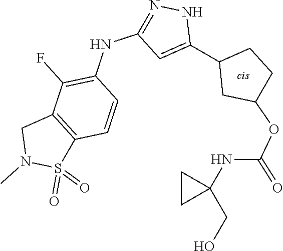 [SMILES: CN1Cc2c(ccc(Nc3cc(C4CCC(OC(=O)NC5(CO)CC5)C4)[nH]n3)c2F)S1(=O)=O]

The titled compound was synthesized in the procedures similar to Example 335. ¹H NMR (500 MHz, DMSO-d₆) δ 11.99 (s, 1H), 8.77 (s, 1H), 8.28 (t, J=8.0 Hz, 1H), 7.54 (d, J=8.6 Hz, 1H), 7.32 (s, 1H), 5.83 (s, 1H), 4.99 (s, 1H), 4.60 (t, J=5.7 Hz, 1H), 4.43 (s, 2H), 3.39 (d, J=5.5 Hz, 2H), 3.13-2.98 (m, 1H), 2.78 (s, 3H), 2.46 (dd, J=14.5, 7.6 Hz, 1H), 2.02 (dd, J=17.1, 9.3 Hz, 1H), 1.96-1.82 (m, 1H), 1.69 (dt, J=15.4, 7.1 Hz, 2H), 1.63-1.53 (m, 1H), 0.63 (t, J=5.5 Hz, 2H), 0.54 (s, 2H). LC-MS (ESI): m/z [M+H]⁺=480.2.

### Example 341: cis-3-(3-((1-methyl-2,2-dioxido-1,3-dihydrobenzo[c]isothiazol-5-yl)amino)-1H-pyrazol-5-yl)cyclopentyl((1s,4s)-4-hydroxycyclohexyl)carbamate

### Step 1: cis-3-(3-((1-methyl-2,2-dioxido-1,3-dihydrobenzo[c]isothiazol-5-yl)amino)-1H-pyrazol-5-yl)cyclopentyl((1s, 4s)-4-hydroxycyclohexyl)carbamate

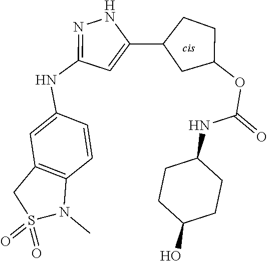 [SMILES: CN1c2ccc(Nc3cc(C4CCC(OC(=O)N[C@H]5CC[C@@H](O)CC5)C4)[nH]n3)cc2CS1(=O)=O]

The titled compound was synthesized in the procedures similar to Example 324. ¹H NMR (500 MHz, DMSO-d₆) δ 11.62 (s, 1H), 8.21 (s, 1H), 7.40 (s, 1H), 7.28-7.20 (m, 1H), 6.98 (d, J=7.6 Hz, 1H), 6.78 (d, J=8.6 Hz, 1H), 5.58 (s, 1H), 4.98 (s, 1H), 4.56 (s, 2H), 4.29 (s, 1H), 3.67 (s, 1H), 3.08-2.99 (m, 1H), 2.95 (s, 3H), 2.45-2.39 (m, 1H), 2.05-1.95 (m, 1H), 1.95-1.80 (m, 1H), 1.80-1.65 (m, 2H), 1.63-1.50 (m, 5H), 1.48-1.34 (m, 4H). LC-MS (ESI): m/z [M+H]⁺=490.2.

### Example 342: cis-3-(3-((1-methyl-2,2-dioxido-1,3-dihydrobenzo[c]isothiazol-5-yl)amino)-1H-pyrazol-5-yl)cyclopentyl(1-(hydroxymethyl)cyclopropyl)carbamate

### Step 1: cis-3-(3-((1-methyl-2,2-dioxido-1,3-dihydrobenzo[c]isothiazol-5-yl)amino)-1H-pyrazol-5-yl)cyclopentyl(1-(hydroxymethyl)cyclopropyl)carbamate

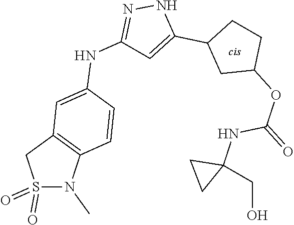 [SMILES: CN1c2ccc(Nc3cc(C4CCC(OC(=O)NC5(CO)CC5)C4)[nH]n3)cc2CS1(=O)=O]

The titled compound was synthesized in the procedures similar to Example 324. ¹H NMR (500 MHz, DMSO-d₆) δ 11.66 (s, 1H), 8.20 (s, 1H), 7.40 (s, 1H), 7.33 (s, 1H), 7.24 (d, J=8.6 Hz, 1H), 6.78 (d, J=8.6 Hz, 1H), 5.57 (s, 1H), 4.98 (s, 1H), 4.58 (d, J=18.3 Hz, 3H), 3.38 (s, 2H), 3.07-2.98 (m, 1H), 2.49-2.38 (m, 1H), 2.06-1.96 (m, 1H), 1.96-1.85 (m, 1H), 1.81-1.66 (m, 3H), 1.65-1.53 (m, 1H), 0.64 (d, J=6.6 Hz, 2H), 0.54 (s, 2H). LC-MS (ESI): m/z [M+H]⁺=462.2.

### Example 343: cis-3-(3-((2,2-dioxido-1,3-dihydrobenzo[c]isothiazol-5-yl)amino)-1H-pyrazol-5-yl)cyclopentyl(1-(oxetan-3-yl)ethyl)carbamate

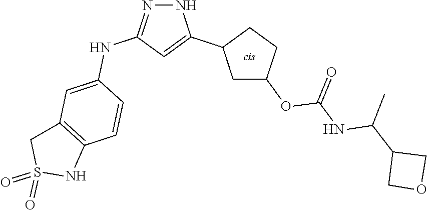 [SMILES: CC(NC(=O)OC1CCC(c2cc(Nc3ccc4c(c3)CS(=O)(=O)N4)n[nH]2)C1)C1COC1]

### Step 1: cis-4-nitrophenyl 3-((1-(4-methoxybenzyl)-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate

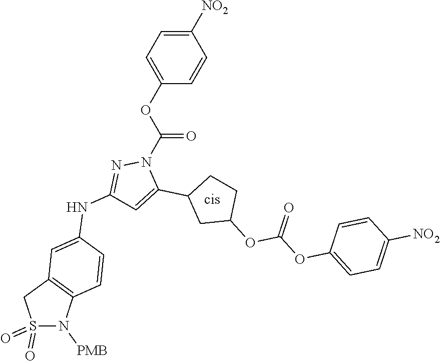

The titled compound was synthesized in the procedures similar to Example 196, step 1-4. LC-MS (ESI): m/z [M+H]⁺=785.2.

### Step 2: cis-3-(3-((1-(4-methoxybenzyl)-2,2-dioxido-1,3-dihydrobenzo[c]isothiazol-5-yl)amino)-1H-pyrazol-5-yl)cyclopentyl(1-(oxetan-3-yl)ethyl)carbamate

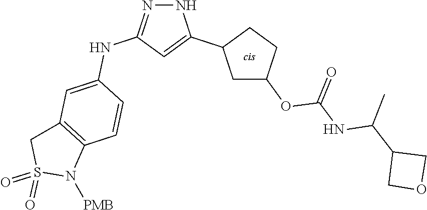

cis-4-nitrophenyl 3-((1-(4-methoxybenzyl)-2,2-dioxido-1,3-dihydrobenzo[c]isothiazol-5-yl)amino)-5-(3-(((4-nitrophenoxy) carbonyl)oxy) cyclopentyl)-1H-pyrazole-1-carboxylate (50 mg, 0.074 mmol), DIEA (95.5 mg, 0.74 mmol), 1-(oxetan-3-yl)ethan-1-amine (37.4 mg, 0.37 mmol) in THF (5 mL) was stirred at 50° C. for 12 h. The mixture was concentrated under reduced pressure. The residue was purified by silica gel column chromatography, eluting with EA to afford the product 50 mg. LC-MS (ESI): m/z [M+H]⁺=582.2.

### Step 3: cis-3-(3-((2,2-dioxido-1,3-dihydrobenzo[c]isothiazol-5-yl)amino)-1H-pyrazol-5-yl)cyclopentyl(1-(oxetan-3-yl)ethyl)carbamate

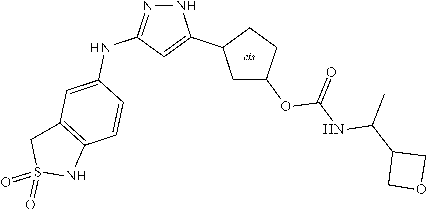 [SMILES: CC(NC(=O)OC1CCC(c2cc(Nc3ccc4c(c3)CS(=O)(=O)N4)n[nH]2)C1)C1COC1]

To a stirring solution of cis-3-(3-((1-(4-methoxybenzyl)-2,2-dioxido-1,3-dihydrobenzo[c]isothiazol-5-yl)amino)-1H-pyrazol-5-yl)cyclopentyl(1-(oxetan-3-yl)ethyl)carbamate (50 mg, 0.086 mmol) in DCM (10 mL) was added TFA (3 mL), The solution was stirred for 12 h at RT. The mixture was concentrated under reduced pressure. The residue was purified by silica gel column chromatography, eluting with EA to afford the product 40 mg. The residue 50 mg was purified by prep HPLC (Waters XSelect C18: RD-CO-094 column, eluting with a gradient of acetonitrile/water containing 0.1% FA, 20%-40%) to afford the product (7.8 mg, 25.2%). ¹H NMR (500 MHz, DMSO-d₆) δ 11.62 (s, 1H), 9.74 (s, 1H), 8.18 (s, 1H), 7.37 (s, 1H), 7.23 (s, 1H), 7.15 (s, 1H), 6.70 (d, J=8.5 Hz, 1H), 5.57 (s, 1H), 4.99 (s, 1H), 4.43 (s, 2H), 3.81 (s, 1H), 3.73 (t, J=7.1 Hz, 2H), 3.43 (d, J=8.6 Hz, 1H), 3.08-2.97 (m, 1H), 2.46-2.40 (m, 1H), 2.20-2.10 (m, 1H), 2.05-1.95 (m, 1H), 1.95-1.85 (m, 1H), 1.80-1.65 (m, 3H), 1.62-1.53 (m, 1H), 1.32 (s, 3H). LC-MS (ESI): m/z [M+H]⁺=462.2.

### Example 344: cis-3-(3-((4-fluoro-2-methyl-1,1-dioxido-2,3-dihydrobenzo[d]isothiazol-5-yl)amino)-1H-pyrazol-5-yl)cyclopentyl(1-(methoxymethyl)cyclopropyl)carbamate

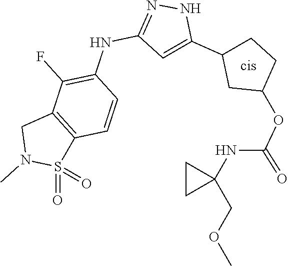 [SMILES: COCC1(NC(=O)OC2CCC(c3cc(Nc4ccc5c(c4F)CN(C)S5(=O)=O)n[nH]3)C2)CC1]

The titled compound was synthesized in the procedures similar to Example 335. ¹H NMR (500 MHz, DMSO-d₆) δ 12.00 (s, 1H), 8.77 (s, 1H), 8.28 (t, J=7.7 Hz, 1H), 7.54 (d, J=8.6 Hz, 1H), 7.40 (s, 1H), 5.84 (s, 1H), 4.99 (s, 1H), 4.43 (s, 2H), 3.32 (s, 2H), 3.23 (s, 3H), 3.05 (dd, J=16.1, 8.0 Hz, 1H), 2.79 (s, 3H), 2.46 (dd, J=14.0, 7.1 Hz, 1H), 2.11-1.97 (m, 1H), 1.94-1.83 (m, 1H), 1.69 (dd, J=15.8, 8.2 Hz, 2H), 1.60-1.55 (m, 1H), 0.62 (s, 4H). LC-MS (ESI): m/z [M+H]⁺=494.2.

### Example 345: cis-3-(3-((4-(hydroxymethyl)-1,1-dioxido-2,3-dihydrobenzo[d]isothiazol-5-yl)amino)-1H-pyrazol-5-yl)cyclopentyl isopropylcarbamate

### Step 1: methyl 5-bromo-3-oxo-2,3-dihydrobenzo[d]isothiazole-4-carboxylate 1,1-dioxide

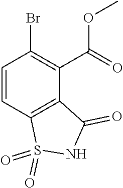 [SMILES: COC(=O)c1c(Br)ccc2c1C(=O)NS2(=O)=O]

The titled compound was synthesized in the procedures similar to Example 1, step 10 to step 11.

LC-MS (ESI): m/z [M+H]⁺=319.9.

### Step 2:5-bromo-4-(hydroxymethyl)-2,3-dihydrobenzo[d]isothiazole 1,1-dioxide

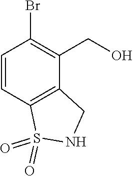 [SMILES: O=S1(=O)NCc2c1ccc(Br)c2CO]

To a solution of methyl 5-bromo-3-oxo-2,3-dihydrobenzo[d]isothiazole-4-carboxylate 1,1-dioxide (1 g, 3.1 mmol) was added BH₃ (15.6 mL, 15.6 mmol). The mixture was stirred at 80° C. for 16 h under a nitrogen atmosphere. The mixture was quenched by MeOH slowly, concentrated under vacuum. The residue was purified by silica gel column chromatography, eluting with EA/PE (0-50%) to afford the product (400 mg, 45.9%). LC-MS (ESI): m/z [M+H]⁺=278.

### Step 3:5-bromo-4-(((tert-butyldimethylsilyl)oxy) methyl)-2,3-dihydrobenzo[d]isothiazole 1,1-dioxide

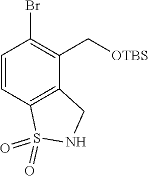 [SMILES: CC(C)(C)[Si](C)(C)OCc1c(Br)ccc2c1CNS2(=O)=O]

To a solution of 5-bromo-4-(hydroxymethyl)-2,3-dihydrobenzo[d]isothiazole 1,1-dioxide (100 mg, 0.36 mmol) in DMF (5 mL) was added imidazole (25 mg, 0.36 mmol) and tert-butylchlorodimethylsilane (109 mg, 0.72 mmol). The mixture was stirred at rt for 16 h. The mixture was quenched by water (50 mL), extracted with EA (3×30 mL). The combined organic layers were dried over Na₂SO₄, filtered and concentrated under vacuum. The residue was purified by silica gel column chromatography, eluting with EA/PE (0-40%) to afford the product (120 mg, 85.1%). LC-MS (ESI): m/z [M+H]⁺=392.

### Step 4: cis-3-(3-((4-(((tert-butyldimethylsilyl)oxy) methyl)-1,1-dioxido-2,3-dihydrobenzo[d]isothiazol-5-yl)amino)-1H-pyrazol-5-yl)cyclopentyl isopropylcarbamate

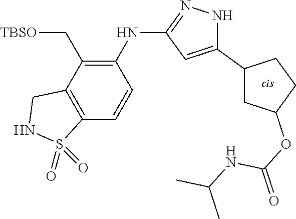 [SMILES: CC(C)NC(=O)OC1CCC(c2cc(Nc3ccc4c(c3CO[Si](C)(C)C(C)(C)C)CNS4(=O)=O)n[nH]2)C1]

To a solution of 5-bromo-4-(((tert-butyldimethylsilyl)oxy) methyl)-2,3-dihydrobenzo[d]isothiazole 1,1-dioxide (100 mg, 0.26 mmol), cis-3-(3-amino-1H-pyrazol-5-yl)cyclopentyl isopropylcarbamate (46 mg, 0.18 mmol), Brettphos Pd G3 (23.6 mg, 0.026 mmol) and K₂CO₃ (107.6 mg, 0.78 mmol) in t-BuOH (5 mL) was stirred at 110° C. for 16 h under a nitrogen atmosphere. The mixture was concentrated under vacuum. The residue was purified by silica gel column chromatography, eluting with EA/PE (0-60%) to afford the product (110 mg, 76.4%). LC-MS (ESI): m/z [M+H]⁺=564.

### Step 5: cis-3-(3-((4-(hydroxymethyl)-1,1-dioxido-2,3-dihydrobenzo[d]isothiazol-5-yl)amino)-1H-pyrazol-5-yl)cyclopentyl isopropylcarbamate

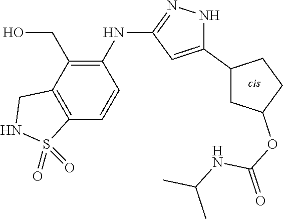 [SMILES: CC(C)NC(=O)OC1CCC(c2cc(Nc3ccc4c(c3CO)CNS4(=O)=O)n[nH]2)C1]

To a solution of cis-3-(3-((4-(((tert-butyldimethylsilyl)oxy) methyl)-1,1-dioxido-2,3-dihydrobenzo[d]isothiazol-5-yl)amino)-1H-pyrazol-5-yl)cyclopentyl isopropylcarbamate (110 mg, 0.19 mmol) in THF (10 mL) was added TBAF (0.23 mL, 0.23 mmol). The mixture was stirred at rt for 4 h. The mixture was concentrated under vacuum. The residue was purified by silica gel column chromatography, eluting with MeOH/DCM (0-10%) to afford the product (48.39 mg, 87.8%). ¹H NMR (500 MHz, DMSO-d₆) δ 11.99 (s, 1H), 8.03 (s, 1H), 7.92-7.80 (m, 1H), 7.59-7.46 (m, 2H), 6.95 (d, J=10 Hz, 1H), 5.84 (s, 1H), 5.41 (t, J=5 Hz, 1H), 5.04-4.94 (m, 1H), 4.54 (d, J=5.5 Hz, 2H), 4.38 (d, J=4.5 Hz, 2H), 3.62-3.50 (m, 1H), 3.13-3.00 (m, 1H), 2.47-2.42 (m, 1H), 2.06-1.98 (m, 1H), 1.96-1.84 (m, 1H), 1.78-1.66 (m, 2H), 1.65-1.55 (m, 1H), 1.07-0.95 (m, 6H). LC-MS (ESI): m/z [M+H]⁺=450.

### Example 346: cis-3-(3-((3,3-difluoro-1-methyl-2,2-dioxido-1,3-dihydrobenzo[c]isothiazol-5-yl)amino)-1H-pyrazol-5-yl)cyclopentyl isopropylcarbamate

### Step 1:5-bromo-3,3-difluoro-1-methyl-1,3-dihydrobenzo[c]isothiazole 2,2-dioxide

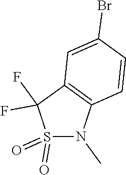

To a mixture of 5-bromo-1-methyl-1,3-dihydrobenzo[c]isothiazole 2,2-dioxide (100 mg, 0.27 mmol) and N-fluoro-N-(phenylsulfonyl)benzenesulfonamide (149 mg, 0.57 mmol) in THF (10 mL) was added LiHMDS (0.65 mL, 0.65 mmol) at −50° C. under a nitrogen atmosphere. The mixture was stirred for 5 h at −50° C. The mixture was quenched by ice water (50 mL), extracted with EA (3×50 mL). The combined organic layers were dried over Na₂SO₄, filtered and concentrated under vacuum. The residue was purified by silica gel column chromatography, eluting with EA/PE (0-60%) to afford the product (95 mg, 56%). LC-MS (ESI): m/z [M+H]⁺=297.9.

### Step 2: cis-3-(3-((3,3-difluoro-1-methyl-2,2-dioxido-1,3-dihydrobenzo[c]isothiazol-5-yl)amino)-1H-pyrazol-5-yl)cyclopentyl isopropylcarbamate

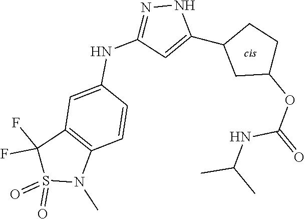 [SMILES: CC(C)NC(=O)OC1CCC(c2cc(Nc3ccc4c(c3)C(F)(F)S(=O)(=O)N4C)n[nH]2)C1]

The titled compound was synthesized in the procedures similar to Example 334. ¹H NMR (500 MHz, DMSO-d₆) δ 11.81 (s, 1H), 8.74 (s, 1H), 8.04 (s, 1H), 7.52 (d, J=10 Hz, 1H), 7.15 (d, J=10 Hz, 1H), 6.97-6.92 (m, 1H), 5.60 (s, 1H), 5.03-4.95 (m, 1H), 3.64-3.52 (m, 1H), 3.32 (s, 3H), 3.10-2.98 (m, 1H), 2.47-2.41 (m, 1H), 2.05-1.97 (m, 1H), 1.95-1.84 (m, 1H), 1.78-1.66 (m, 2H), 1.63-1.54 (m, 1H), 1.08-0.96 (m, 6H). LC-MS (ESI): m/z [M+H]⁺=470.2.

### Example 347: cis-3-(3-((4-fluoro-1-methyl-2,2-dioxido-1,3-dihydrobenzo[c]isothiazol-5-yl)amino)-1H-pyrazol-5-yl)cyclopentyl((1s,4s)-4-hydroxycyclohexyl)carbamate

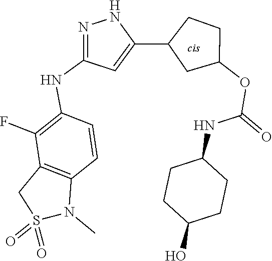 [SMILES: CN1c2ccc(Nc3cc(C4CCC(OC(=O)N[C@H]5CC[C@@H](O)CC5)C4)[nH]n3)c(F)c2CS1(=O)=O]

The titled compound was synthesized in the procedures similar to Example 327. ¹H NMR (500 MHz, DMSO-d₆) δ 11.69 (s, 1H), 8.04 (t, J=8.3 Hz, 1H), 7.92 (s, 1H), 6.98 (d, J=7.6 Hz, 1H), 6.68 (d, J=8.7 Hz, 1H), 5.68 (s, 1H), 4.99 (s, 1H), 4.72 (s, 2H), 4.29 (s, 1H), 3.66 (s, 1H), 3.29-3.24 (m, 1H), 3.05-2.95 (m, 4H), 2.46-2.42 (m, 1H), 2.05-1.85 (m, 2H), 1,75-1.66 (m, 2H), 1,63-1.56 (m, 5H), 1.48-1.35 (m, 4H). LC-MS (ESI): m/z [M+H]⁺=508.3.

### Example 348: cis-3-(3-((4-fluoro-1-methyl-2,2-dioxido-1,3-dihydrobenzo[c]isothiazol-5-yl)amino)-1H-pyrazol-5-yl)cyclopentyl((S)-1-hydroxypropan-2-yl)carbamate

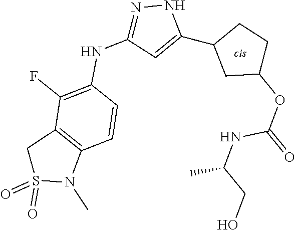 [SMILES: C[C@@H](CO)NC(=O)OC1CCC(c2cc(Nc3ccc4c(c3F)CS(=O)(=O)N4C)n[nH]2)C1]

The titled compound was synthesized in the procedures similar to Example 327. ¹H NMR (500 MHz, DMSO-d₆) δ 11.71 (s, 1H), 8.03 (s, 1H), 7.91 (s, 1H), 6.80 (s, 1H), 6.68 (d, J=8.7 Hz, 1H), 5.68 (s, 1H), 4.99 (s, 1H), 4.72 (s, 2H), 4.61 (t, J=5.6 Hz, 1H), 3.51-3.45 (m, 1H), 3.38-3.33 (m, 1H), 3.23-3.15 (m, 1H), 3.07-2.97 (m, 4H), 2.48-2.43 (m, 1H), 2.03-1.85 (m, 2H), 1.76-1.55 (m, 3H), 1.04-0.96 (m, 3H). LC-MS (ESI): m/z [M+H]⁺=468.3.

### Example 349: cis-3-(3-((4-fluoro-1-methyl-2,2-dioxido-1,3-dihydrobenzo[c]isothiazol-5-yl)amino)-1H-pyrazol-5-yl)cyclopentyl(3-methyltetrahydrofuran-3-yl)carbamate

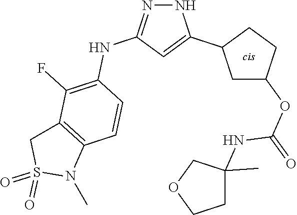 [SMILES: CN1c2ccc(Nc3cc(C4CCC(OC(=O)NC5(C)CCOC5)C4)[nH]n3)c(F)c2CS1(=O)=O]

The titled compound was synthesized in the procedures similar to Example 327 in a racemic form, which was further separated by Chiral Pre-HPLC to give:

Enantiomer 1 (Example 349a, 100% ee); Retention time: 5.812 min. ¹H NMR (500 MHz, DMSO-d₆) δ 11.70 (s, 1H), 8.04 (s, 1H), 7.92 (s, 1H), 7.23 (s, 1H), 6.68 (d, J=8.8 Hz, 1H), 5.68 (s, 1H), 4.99 (s, 1H), 4.72 (s, 2H), 3.84-3.78 (m, 1H), 3.76-3.72 (m, 2H), 3.43 (d, J=8.6 Hz, 1H), 3.07-2.95 (m, 4H), 2.48-2.42 (m, 1H), 2.20-2.10 (m, 1H), 2.04-1.86 (m, 2H), 1.77-1.65 (m, 3H), 1.63-1.53 (m, 1H), 1.32 (s, 3H). LC-MS (ESI): m/z [M+H]⁺=494.3.

Enantiomer 2 (Example 349b, 100% ee); Retention time: 7.755 min. ¹H NMR (500 MHz, DMSO-d₆) δ 11.70 (s, 1H), 8.04 (s, 1H), 7.92 (s, 1H), 7.23 (s, 1H), 6.68 (d, J=8.8 Hz, 1H), 5.68 (s, 1H), 4.99 (s, 1H), 4.72 (s, 2H), 3.84-3.78 (m, 1H), 3.76-3.72 (m, 2H), 3.43 (d, J=8.6 Hz, 1H), 3.07-2.95 (m, 4H), 2.48-2.42 (m, 1H), 2.20-2.10 (m, 1H), 2.04-1.86 (m, 2H), 1.77-1.65 (m, 3H), 1.63-1.53 (m, 1H), 1.32 (s, 3H). LC-MS (ESI): m/z [M+H]⁺=494.3.

Enantiomer 3 (Example 349c, 96.15% ee); Retention time: 8.943 min. ¹H NMR (500 MHz, DMSO-d₆) δ 11.70 (s, 1H), 8.04 (s, 1H), 7.92 (s, 1H), 7.23 (s, 1H), 6.68 (d, J=8.8 Hz, 1H), 5.68 (s, 1H), 4.99 (s, 1H), 4.72 (s, 2H), 3.84-3.78 (m, 1H), 3.76-3.72 (m, 2H), 3.43 (d, J=8.6 Hz, 1H), 3.07-2.95 (m, 4H), 2.48-2.42 (m, 1H), 2.20-2.10 (m, 1H), 2.04-1.86 (m, 2H), 1.77-1.65 (m, 3H), 1.63-1.53 (m, 1H), 1.32 (s, 3H). LC-MS (ESI): m/z [M+H]⁺=494.3.

Enantiomer 4 (Example 349d, 99.86% ee); Retention time: 12.233 min. ¹H NMR (500 MHz, DMSO-d₆) δ 11.70 (s, 1H), 8.04 (s, 1H), 7.92 (s, 1H), 7.23 (s, 1H), 6.68 (d, J=8.8 Hz, 1H), 5.68 (s, 1H), 4.99 (s, 1H), 4.72 (s, 2H), 3.84-3.78 (m, 1H), 3.76-3.72 (m, 2H), 3.43 (d, J=8.6 Hz, 1H), 3.07-2.95 (m, 4H), 2.48-2.42 (m, 1H), 2.20-2.10 (m, 1H), 2.04-1.86 (m, 2H), 1.77-1.65 (m, 3H), 1.63-1.53 (m, 1H), 1.32 (s, 3H). LC-MS (ESI): m/z [M+H]⁺=494.3.

Chiral analytical method: Column: CHIRALPAK IE 4.6 mm×250 mm, 5 μm; Mobile phase: A for MTBE (0.1% 2M NH₃ MeOH) and B for MEOH/DCM (50/50); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MEOH/DCM (50/50); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 350: cis-3-(3-((4-fluoro-1-methyl-2,2-dioxido-1,3-dihydrobenzo[c]isothiazol-5-yl)amino)-1H-pyrazol-5-yl)cyclopentyl(1-(hydroxymethyl)cyclopropyl)carbamate

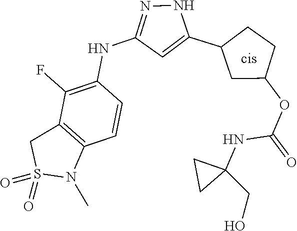 [SMILES: CN1c2ccc(Nc3cc(C4CCC(OC(=O)NC5(CO)CC5)C4)[nH]n3)c(F)c2CS1(=O)=O]

The titled compound was synthesized in the procedures similar to Example 327. ¹H NMR (500 MHz, DMSO-d₆) δ 11.69 (s, 1H), 8.02 (s, 1H), 7.91 (s, 1H), 7.32 (s, 1H), 6.68 (d, J=8.7 Hz, 1H), 5.68 (s, 1H), 4.98 (s, 1H), 4.72 (s, 2H), 4.60 (t, J=5.7 Hz, 1H), 3.38 (d, J=5.4 Hz, 2H), 3.08-2.96 (m, 4H), 2.48-2.43 (m, 1H), 2.05-1.82 (m, 2H), 1.76-1.54 (m, 3H), 0.63 (d, J=6.5 Hz, 2H), 0.54 (s, 2H). LC-MS (ESI): m/z [M+H]⁺=480.3.

### Example 351: cis-3-(3-((4-fluoro-2-methyl-1,1-dioxido-2,3-dihydrobenzo[d]isothiazol-5-yl)amino)-1H-pyrazol-5-yl)cyclopentyl((S)-1-hydroxypropan-2-yl)carbamate

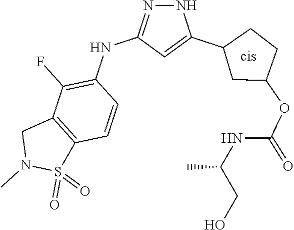 [SMILES: C[C@@H](CO)NC(=O)OC1CCC(c2cc(Nc3ccc4c(c3F)CN(C)S4(=O)=O)n[nH]2)C1]

The titled compound was synthesized in the procedures similar to Example 335. ¹H NMR (500 MHz, DMSO-d₆) δ 12.00 (s, 1H), 8.77 (s, 1H), 8.29 (t, J=8.0 Hz, 1H), 7.54 (d, J=8.6 Hz, 1H), 6.80 (s, 1H), 5.84 (s, 1H), 5.00 (s, 1H), 4.62 (t, J=5.5 Hz, 1H), 4.43 (s, 2H), 3.59-3.43 (m, 1H), 3.24-3.13 (m, 1H), 3.12-3.02 (m, 1H), 2.78 (s, 3H), 2.41 (dd, J=24.9, 17.6 Hz, 4H), 2.05-2.00 (m, 1H), 1.97-1.83 (m, 1H), 1.74-1.68 (m, 2H), 1.62-1.57 (m, 1H), 1.00 (d, J=6.4 Hz, 3H). LC-MS (ESI): m/z [M+H]⁺=468.2.

### Example 352: cis-3-(3-((4-fluoro-2-methyl-1,1-dioxido-2,3-dihydrobenzo[d]isothiazol-5-yl)amino)-1H-pyrazol-5-yl)cyclopentyl(3-methyloxetan-3-yl)carbamate

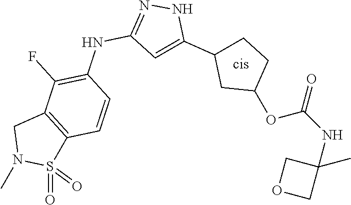

The titled compound was synthesized in the procedures similar to Example 328 in a racemic form, which was further separated by Chiral Prep-HPLC to give:

Enantiomer 1 (Example 352a, 100% ee); Retention time: 9.67 min. ¹H NMR (500 MHz, DMSO-d₆) δ 12.02 (s, 1H), 8.78 (s, 1H), 8.29 (t, J=5 Hz, 1H), 7.68-7.58 (m, 1H), 7.54 (d, J=5 Hz, 1H), 5.84 (s, 1H), 5.06-4.97 (m, 1H), 4.61-4.50 (m, 2H), 4.43 (s, 2H), 4.28-4.19 (m, 2H), 3.14-3.03 (m, 1H), 2.79 (s, 3H), 2.49-2.45 (m, 1H), 2.08-2.00 (m, 1H), 1.97-1.86 (m, 1H), 1.79-1.66 (m, 2H), 1.66-1.58 (m, 1H), 1.47 (s, 3H). LC-MS (ESI): m/z [M+H]⁺=480.2.

Enantiomer 2 (Example 352b, 100% ee); Retention time: 16.52 min. ¹H NMR (500 MHz, DMSO-d₆) δ 12.02 (s, 1H), 8.78 (s, 1H), 8.29 (t, J=5 Hz, 1H), 7.68-7.58 (m, 1H), 7.54 (d, J=5 Hz, 1H), 5.84 (s, 1H), 5.06-4.97 (m, 1H), 4.61-4.50 (m, 2H), 4.43 (s, 2H), 4.28-4.19 (m, 2H), 3.14-3.03 (m, 1H), 2.79 (s, 3H), 2.49-2.45 (m, 1H), 2.08-2.00 (m, 1H), 1.97-1.86 (m, 1H), 1.79-1.66 (m, 2H), 1.66-1.58 (m, 1H), 1.47 (s, 3H). LC-MS (ESI): m/z [M+H]⁺=480.2.

Chiral analytical method: Column: CHIRALPAK IE, 4.6*250 mm 5 μm; Mobile phase: A for MTBE (0.1% 2 M NH₃. MeOH) and B for MeOH: DCM=50:50; Gradient: Mobile Phase A: Mobile Phase B=10:90 (v/v); HPLC Equipment: HPLC-Agilent, Back pressure: 100 bar; Column temperature: 35° C.

Chiral Prep-HPLC Condition: CHIRALPAK IE, 4.6*250 mm 5 μm; Mobile phase: A for MTBE (0.2% 2 M NH₃. MeOH) and B for MeOH: DCM=50: 50; Gradient: Mobile Phase A: Mobile Phase B=10:90 (v/v); Flow Rate: 18 mL/min, Wave Length: UV 254 nm and 280 nm, Prep-HPLC Equipment: Prep-HPLC-Gilson, Back pressure: 100 bar; Column temperature: 25° C.

### Example 353: cis-3-(3-((7-methyl-1,1-dioxido-2,3-dihydrobenzo[d]isothiazol-6-yl)amino)-1H-pyrazol-5-yl)cyclopentyl isopropylcarbamate

### Step 1:6-bromo-7-methyl-2,3-dihydrobenzo[d]isothiazole 1,1-dioxide

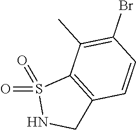

The titled compound was synthesized in the procedures similar to Example 1. LC-MS (ESI): m/z [M+H]⁺=261.9.

### Step 2: cis-3-(3-((7-methyl-1,1-dioxido-2,3-dihydrobenzo[d]isothiazol-6-yl)amino)-1H-pyrazol-5-yl)cyclopentyl isopropylcarbamate

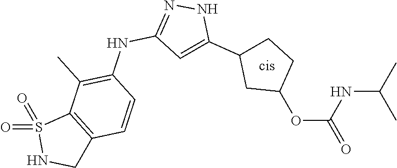 [SMILES: Cc1c(Nc2cc(C3CCC(OC(=O)NC(C)C)C3)[nH]n2)ccc2c1S(=O)(=O)NC2]

The titled compound was synthesized in the procedures similar to Example 34. ¹H NMR (500 MHz, DMSO-d₆) δ=11.86 (s, 1H), 7.94 (s, 1H), 7.66 (t, J=5.0, 1H), 7.60 (s, 1H), 7.16 (d, J=8.5, 1H), 6.93 (d, J=7.4, 1H), 5.76 (s, 1H), 5.00 (m, 1H), 4.21 (d, J=5.0, 2H), 3.57 (m, 1H), 3.04 (m, 1H), 2.45 (m, 1H), 2.38 (s, 3H), 2.02 (m, 1H), 1.97-1.85 (m, 1H), 1.69 (m, 2H), 1.61 (m, 1H), 1.10-0.95 (m, 6H).

LC-MS (ESI): m/z [M+H]⁺=434.34.

### Example 354: cis-3-(3-((1-methyl-2,2-dioxido-1,3-dihydrobenzo[c]isothiazol-5-yl)amino)-1H-pyrazol-5-yl)cyclopentyl(2S,3R)-3-hydroxy-2-methylazetidine-1-carboxylate

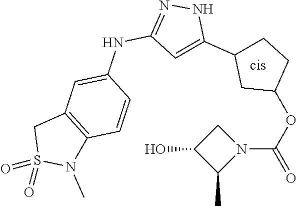 [SMILES: C[C@H]1[C@H](O)CN1C(=O)OC1CCC(c2cc(Nc3ccc4c(c3)CS(=O)(=O)N4C)n[nH]2)C1]

The titled compound was synthesized in the procedures similar to Example 324. ¹H NMR (500 MHz, DMSO-d₆) δ 11.68 (s, 1H), 8.23 (d, J=7.7 Hz, 1H), 7.40 (s, 1H), 7.25 (d, J=8.5 Hz, 1H), 6.78 (d, J=8.6 Hz, 1H), 5.63 (s, 1H), 5.57 (d, J=3.4 Hz, 1H), 5.00 (d, J=2.6 Hz, 1H), 4.56 (s, 2H), 3.95-3.85 (m, 3H), 3.51-3.46 (m, 1H), 3.10-3.00 (m, 1H), 2.95 (s, 3H), 2.46-2.37 (m, 1H), 2.08-1.97 (m, 1H), 1.92-1.81 (m, 1H), 1.79-1.67 (m, 2H), 1.62-1.53 (m, 1H), 1.34-1.14 (m, 3H). LC-MS (ESI): m/z [M+H]⁺=462.2.

### Example 355: cis-3-(3-((4-fluoro-1-methyl-2,2-dioxido-1,3-dihydrobenzo[c]isothiazol-5-yl)amino)-1H-pyrazol-5-yl)cyclopentyl(2S,3R)-3-hydroxy-2-methylazetidine-1-carboxylate

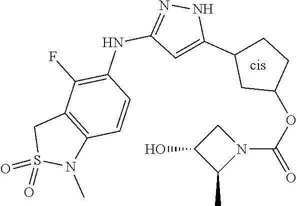 [SMILES: C[C@H]1[C@H](O)CN1C(=O)OC1CCC(c2cc(Nc3ccc4c(c3F)CS(=O)(=O)N4C)n[nH]2)C1]

The titled compound was synthesized in the procedures similar to Example 327. ¹H NMR (500 MHz, DMSO-d₆) δ 11.73 (s, 1H), 8.03 (s, 1H), 7.93 (s, 1H), 6.68 (d, J=8.7 Hz, 1H), 5.67 (d, J=2.8 Hz, 1H), 5.62 (d, J=5.8 Hz, 1H), 5.00 (s, 1H), 4.72 (s, 2H), 3.95-3.85 (m, 3H), 3.55-3.45 (m, 1H), 3.09-3.02 (m, 1H), 2.99 (s, 3H), 2.46-2.39 (m, 1H), 2.03-1.83 (m, 2H), 1.78-1.59 (m, 3H), 1.28-1.22 (m, 3H). LC-MS (ESI): m/z [M+H]⁺=480.3.

### Example 356: cis-3-(3-((3,3,4-trifluoro-2,2-dioxido-1,3-dihydrobenzo[c]isothiazol-5-yl)amino)-1H-pyrazol-5-yl)cyclopentyl isopropylcarbamate

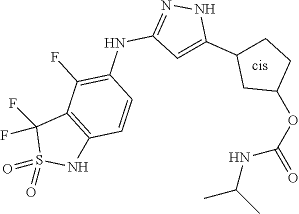 [SMILES: CC(C)NC(=O)OC1CCC(c2cc(Nc3ccc4c(c3F)C(F)(F)S(=O)(=O)N4)n[nH]2)C1]

The titled compound was synthesized in the procedures similar to Example 334. ¹H NMR (500 MHz, DMSO-d₆) δ 11.80 (s, 1H), 8.34-8.17 (m, 2H), 6.93 (d, J=5 Hz, 1H), 6.76 (d, J=5 Hz, 1H), 5.69 (s, 1H), 5.04-4.94 (m, 1H), 3.64-3.51 (m, 1H), 3.10-2.97 (m, 1H), 2.54-2.51 (m, 1H), 2.47-2.41 (m, 1H), 2.05-1.96 (m, 1H), 1.95-1.83 (m, 1H), 1.78-1.63 (m, 2H), 1.62-1.52 (m, 1H), 1.08-0.97 (m, 6H). LC-MS (ESI): m/z [M+H]⁺=474.1.

### Example 357: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-(oxetan-3-yl)ethyl)carbamate

### Step 1: cis-4-nitrophenyl 3-((2-cyclopropyl-4-fluoro-1,1-dioxido-2,3-dihydrobenzo[d]isothiazol-5-yl)amino)-5-(3-(((4-nitrophenoxy) carbonyl)oxy) cyclopentyl)-1H-pyrazole-1-carboxylate

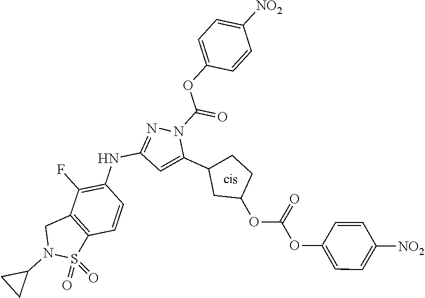 [SMILES: O=C(Oc1ccc([N+](=O)[O-])cc1)OC1CCC(c2cc(Nc3ccc4c(c3F)CN(C3CC3)S4(=O)=O)nn2C(=O)Oc2ccc([N+](=O)[O-])cc2)C1]

The titled compound was synthesized in the procedures similar to example 196. LC-MS (ESI): m/z [M+H]⁺=723.25.

### Step 2: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-(oxetan-3-yl)ethyl)carbamate

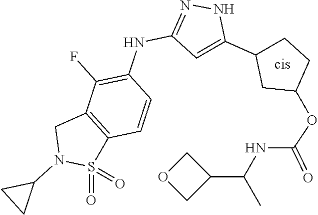 [SMILES: CC(NC(=O)OC1CCC(c2cc(Nc3ccc4c(c3F)CN(C3CC3)S4(=O)=O)n[nH]2)C1)C1COC1]

The titled compound was synthesized in the procedures similar to example 196. ¹H NMR (500 MHz, DMSO-d₆) δ 12.01 (s, 1H), 8.76 (d, J=9.9 Hz, 1H), 8.28 (s, 1H), 7.52 (d, J=8.6 Hz, 1H), 7.07 (d, J=8.2 Hz, 1H), 5.83 (s, 1H), 5.08-4.95 (m, 1H), 4.62-4.47 (m, 4H), 4.34 (t, J=6.0 Hz, 1H), 4.24 (t, J=6.1 Hz, 1H), 3.90-3.75 (m, 1H), 3.15-3.01 (m, 1H), 2.95-2.85 (m, 1H), 2.48-2.44 (m, 2H), 2.08-2.00 (m, 1H), 1.97-1.87 (m, 1H), 1.80-1.67 (m, 2H), 1.66-1.57 (m, 1H), 1.00-0.91 (m, 3H), 0.88-0.82 (m, 2H), 0.78-0.70 (m, 2H). LC-MS (ESI): m/z [M+H]⁺=520.29.

### Example 358: cis-3-(3-((2-cyclopropyl-4-fluoro-1,1-dioxido-2,3-dihydrobenzo[d]isothiazol-5-yl)amino)-1H-pyrazol-5-yl)cyclopentyl(3-methyltetrahydrofuran-3-yl)carbamate

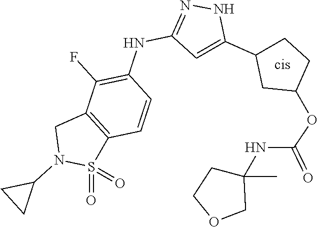 [SMILES: CC1(NC(=O)OC2CCC(c3cc(Nc4ccc5c(c4F)CN(C4CC4)S5(=O)=O)n[nH]3)C2)CCOC1]

The titled compound was synthesized in the procedures similar to example 357. ¹H NMR (500 MHz, DMSO-d₆) δ 12.01 (s, 1H), 8.77 (s, 1H), 8.28 (s, 1H), 7.53 (dd, J=8.8, 4.1 Hz, 1H), 7.23 (s, 1H), 5.83 (s, 1H), 5.10-4.92 (m, 1H), 4.60-4.47 (m, 2H), 3.81 (s, 1H), 3.72 (d, J=4.7 Hz, 2H), 3.47-3.39 (m, 1H), 3.12-3.00 (m, 1H), 2.57-2.53 (m, 1H), 2.47-2.42 (m, 1H), 2.20-2.12 (m, 1H), 2.01-1.98 (m, 1H), 1.97-1.86 (m, 1H), 1.80-1.66 (m, 3H), 1.65-1.53 (m, 1H), 1.38-1.25 (m, 3H), 0.89-0.80 (m, 2H), 0.79-0.68 (m, 2H). LC-MS (ESI): m/z [M+H]⁺=520.29.

### Example 359: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-hydroxy-2-methylpropan-2-yl)carbamate

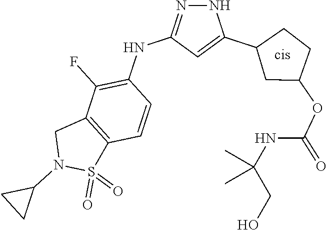 [SMILES: CC(C)(CO)NC(=O)OC1CCC(c2cc(Nc3ccc4c(c3F)CN(C3CC3)S4(=O)=O)n[nH]2)C1]

The titled compound was synthesized in the procedures similar to Example 357 in a racemic form, which was further separated by Chiral Prep-HPLC to give:

Enantiomer 1 (Example 359a, 100% ee); Retention time: 5.715 min. ¹H NMR (500 MHz, DMSO-d₆) δ 12.00 (s, 1H), 8.78 (s, 1H), 8.29 (t, J=5 Hz, 1H), 7.52 (d, J=10 Hz, 1H), 6.47 (s, 1H), 5.83 (s, 1H), 5.02-4.96 (m, 1H), 4.71-4.68 (m, 1H), 4.52 (s, 2H), 3.31-3.27 (m, 1H), 3.08-3.01 (m, 1H), 2.48-2.43 (m, 3H), 2.06-1.97 (m, 1H), 1.95-1.85 (m, 1H), 1.77-1.65 (m, 2H), 1.63-1.54 (m, 1H), 1.14 (m, 6H), 0.87-0.82 (m, 2H), 0.76-0.71 (m, 2H). LC-MS (ESI): m/z [M+H]⁺=508.2.

Enantiomer 2 (Example 359b, 100% ee); Retention time: 12.16 min. ¹H NMR (500 MHz, DMSO-d₆) δ 12.00 (s, 1H), 8.78 (s, 1H), 8.29 (t, J=5 Hz, 1H), 7.52 (d, J=10 Hz, 1H), 6.47 (s, 1H), 5.83 (s, 1H), 5.02-4.96 (m, 1H), 4.71-4.68 (m, 1H), 4.52 (s, 2H), 3.31-3.27 (m, 1H), 3.08-3.01 (m, 1H), 2.48-2.43 (m, 3H), 2.06-1.97 (m, 1H), 1.95-1.85 (m, 1H), 1.77-1.65 (m, 2H), 1.63-1.54 (m, 1H), 1.14 (m, 6H), 0.87-0.82 (m, 2H), 0.76-0.71 (m, 2H). LC-MS (ESI): m/z [M+H]⁺=508.2.

Chiral analytical method: Column: CHIRALPAK IE, 4.6*250 mm 5 μm; Mobile phase: A for MTBE (0.1% 2 M NH₃. MeOH) and B for MeOH: DCM=50:50; Gradient: Mobile Phase A: Mobile Phase B=40:60 (v/v); HPLC Equipment: HPLC-Agilent, Back pressure: 100 bar; Column temperature: 35° C.

Chiral Prep-HPLC Condition: CHIRALPAK IE, 20*250 mm 5 μm; Mobile phase: A for MTBE (0.2% 2 M NH₃. MeOH) and B for MeOH: DCM=50:50; Gradient: Mobile Phase A: Mobile Phase B=40:60 (v/v); Flow Rate: 18 mL/min, Wave Length: UV 254 nm and 280 nm, Prep-HPLC Equipment: Prep-HPLC-Gilson, Back pressure: 100 bar; Column temperature: 25° C.

### Example 360: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-(hydroxymethyl)cyclopropyl)carbamate

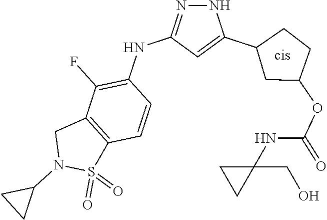 [SMILES: O=C(NC1(CO)CC1)OC1CCC(c2cc(Nc3ccc4c(c3F)CN(C3CC3)S4(=O)=O)n[nH]2)C1]

The titled compound was synthesized in the procedures similar to Example 357. ¹H NMR (500 MHz, DMSO-d₆) δ 11.99 (s, 1H), 8.77 (s, 1H), 8.28 (t, J=5 Hz, 1H), 7.52 (d, J=10 Hz, 1H), 7.32 (s, 1H), 5.83 (s, 1H), 5.02-4.94 (m, 1H), 4.70 (d, J=5 Hz, 1H), 4.52 (s, 2H), 3.34-3.32 (m, 2H), 3.12-3.02 (m, 1H), 2.49-2.42 (m, 2H), 2.06-1.97 (m, 1H), 1.95-1.82 (m, 1H), 1.78-1.63 (m, 2H), 1.62-1.54 (m, 1H), 0.88-0.81 (m, 2H), 0.76-0.70 (m, 2H), 0.66-0.60 (m, 2H), 0.57-0.51 (m, 2H). LC-MS (ESI): m/z [M+H]⁺=506.2.

### Example 361: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-methoxypropan-2-yl)carbamate

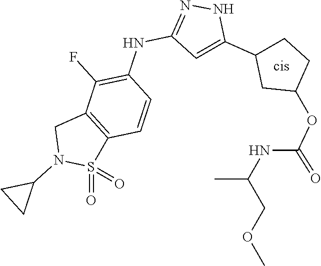 [SMILES: COCC(C)NC(=O)OC1CCC(c2cc(Nc3ccc4c(c3F)CN(C3CC3)S4(=O)=O)n[nH]2)C1]

The titled compound was synthesized in the procedures similar to Example 357. ¹H NMR (500 MHz, DMSO-d₆) δ 12.00 (s, 1H), 8.77 (s, 1H), 8.28 (t, J=5 Hz, 1H), 7.52 (d, J=10 Hz, 1H), 6.94 (d, J=5 Hz, 1H), 5.83 (s, 1H), 5.02-4.95 (m, 1H), 4.52 (s, 2H), 3.70-3.55 (m, 1H), 3.28-3.19 (m, 4H), 3.16-3.02 (m, 2H), 3.48-3.45 (m, 2H), 2.09-1.98 (m, 1H), 1.97-1.85 (m, 1H), 1.81-1.65 (m, 2H), 1.64-1.55 (m, 1H), 1.09-0.94 (m, 3H), 0.90-0.81 (m, 2H), 0.80-0.71 (m, 2H). LC-MS (ESI): m/z [M+H]⁺=508.2.

### Example 362: cis-3-(3-((1-cyclopropyl-4-fluoro-2,2-dioxido-1,3-dihydrobenzo[c]isothiazol-5-yl)amino)-1H-pyrazol-5-yl)cyclopentyl(3-methyloxetan-3-yl)carbamate

### Step 1:5-bromo-1-cyclopropyl-4-fluoro-1,3-dihydrobenzo[c]isothiazole 2,2-dioxide

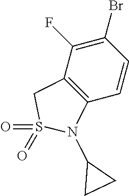 [SMILES: O=S1(=O)Cc2c(ccc(Br)c2F)N1C1CC1]

The titled compound was synthesized in the procedures similar to Example 251. LC-MS (ESI): m/z [M+H]⁺=305.9.

### Step 2: cis-3-(3-amino-1H-pyrazol-5-yl)cyclopentyl(3-methyloxetan-3-yl)carbamate

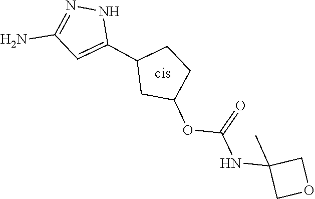 [SMILES: CC1(NC(=O)OC2CCC(c3cc(N)n[nH]3)C2)COC1]

The titled compound was synthesized in the procedures similar to Example 34, step 3-4. LC-MS (ESI): m/z [M+H]⁺=281.1.

### Step 3: cis-3-(3-((1-cyclopropyl-4-fluoro-2,2-dioxido-1,3-dihydrobenzo[c]isothiazol-5-yl)amino)-1H-pyrazol-5-yl)cyclopentyl(3-methyloxetan-3-yl)carbamate

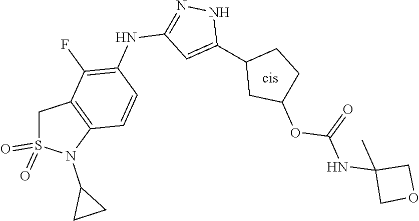 [SMILES: CC1(NC(=O)OC2CCC(c3cc(Nc4ccc5c(c4F)CS(=O)(=O)N5C4CC4)n[nH]3)C2)COC1]

The titled compound was synthesized in the procedures similar to Example 34. ¹H NMR (500 MHz, DMSO-d₆) δ=11.74 (s, 1H), 8.04 (s, 1H), 7.94 (s, 1H), 7.62 (s, 1H), 6.83 (d, J=8.7, 1H), 5.69 (s, 1H), 5.01 (m, 1H), 4.70 (s, 2H), 4.56 (s, 2H), 4.24 (d, J=4.9, 2H), 3.09-2.93 (m, 1H), 2.48-2.43 (m, 2H), 2.01 (m, 1H), 1.90 (m, 1H), 1.72 (m, 2H), 1.61 (m, 1H), 1.47 (s, 3H), 0.99-0.90 (m, 2H), 0.90-0.83 (m, 2H). LC-MS (ESI): m/z [M+H]⁺=506.29.

### Example 363: cis-3-(3-((1-cyclopropyl-4-fluoro-2,2-dioxido-1,3-dihydrobenzo[c]isothiazol-5-yl)amino)-1H-pyrazol-5-yl)cyclopentyl(1-hydroxy-2-methylpropan-2-yl)carbamate

### Step 1: cis-3-(3-amino-1H-pyrazol-5-yl)cyclopentyl(1-hydroxy-2-methylpropan-2-yl)carbamate

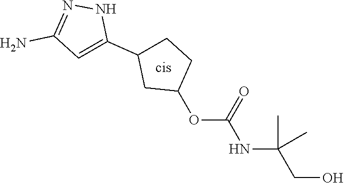 [SMILES: CC(C)(CO)NC(=O)OC1CCC(c2cc(N)n[nH]2)C1]

The titled compound was synthesized in the procedures similar to Example 34, step 3-4. LC-MS (ESI): m/z [M+H]⁺=283.2.

### Step 2: cis-3-(3-((1-cyclopropyl-4-fluoro-2,2-dioxido-1,3-dihydrobenzo[c]isothiazol-5-yl)amino)-1H-pyrazol-5-yl)cyclopentyl(1-hydroxy-2-methylpropan-2-yl)carbamate

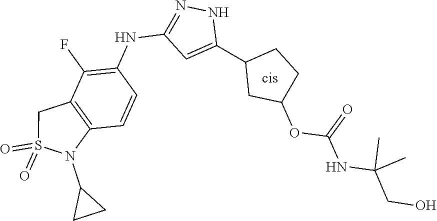 [SMILES: CC(C)(CO)NC(=O)OC1CCC(c2cc(Nc3ccc4c(c3F)CS(=O)(=O)N4C3CC3)n[nH]2)C1]

The titled compound was synthesized in the procedures similar to Example 362 in a racemic form, which was further separated by Chiral Prep-HPLC to give:

Enantiomer 1 (Example 363a, 100% ee); Retention time: 4.382 min. ¹H NMR (500 MHz, DMSO-d₆) δ=11.71 (s, 1H), 8.05 (t, J=8.7, 1H), 7.93 (s, 1H), 6.83 (d, J=8.7, 1H), 6.47 (s, 1H), 5.69 (s, 1H), 4.96 (m, 1H), 4.73-4.65 (m, 3H), 3.32-3.30 (m, 2H), 3.02 (m, 1H), 2.49-2.41 (m, 2H), 2.00 (m, 1H), 1.94-1.82 (m, 1H), 1.76-1.63 (m, 2H), 1.58 (m, 1H), 1.14 (s, 6H), 0.96-0.91 (m, 2H), 0.91-0.85 (m, 2H). LC-MS (ESI): m/z [M+H]⁺=508.35.

Enantiomer 2 (Example 363b, 100% ee); Retention time: 5.595 min. ¹H NMR (500 MHz, DMSO-d₆) δ=11.71 (s, 1H), 8.05 (s, 1H), 7.93 (s, 1H), 6.83 (d, J=8.7, 1H), 6.47 (s, 1H), 5.69 (s, 1H), 4.96 (m, 1H), 4.77-4.62 (m, 3H), 3.32 (s, 2H), 3.07-2.96 (m, 1H), 2.49-2.40 (m, 2H), 2.05-1.94 (m, 1H), 1.94-1.78 (m, 1H), 1.77-1.63 (m, 2H), 1.58 (m, 1H), 1.13 (s, 6H), 0.97-0.91 (m, 2H), 0.91-0.85 (m, 2H). LC-MS (ESI): m/z [M+H]⁺=508.30.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=40: 60 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=40: 60 (v/v); Flow Rate: 18 mL/min, Wavelength: UV 270 nm and 310 nm, Prep-HPLC Equipment: Prep-HPLC-Gilson; Column temperature: 25° C.

### Example 364: cis-3-(3-((4-fluoro-1,1-dioxido-2,3-dihydrobenzo[d]isothiazol-5-yl)amino)-1H-pyrazol-5-yl)cyclopentyl(1-methoxy-2-methylpropan-2-yl)carbamate

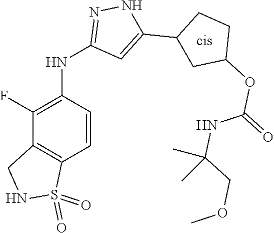 [SMILES: COCC(C)(C)NC(=O)OC1CCC(c2cc(Nc3ccc4c(c3F)CNS4(=O)=O)n[nH]2)C1]

The titled compound was synthesized in the procedures similar to Example 328. ¹H NMR (500 MHz, DMSO-d₆) δ=11.98 (s, 1H), 8.72 (s, 1H), 8.27 (t, J=7.9, 1H), 7.69 (s, 1H), 7.48 (d, J=8.6, 1H), 6.63 (s, 1H), 5.83 (s, 1H), 4.98 (m, 1H), 4.41 (s, 2H), 3.31 (s, 1H), 3.24 (s, 3H), 3.10-3.01 (m, 1H), 2.48-2.41 (m, 1H), 2.02 (m, 1H), 1.90 (m, 1H), 1.77-1.66 (m, 2H), 1.58 (m, 1H), 1.16 (s, 6H). LC-MS (ESI): m/z [M+H]⁺=482.32.

### Example 365: cis-3-(3-((4-fluoro-7-(hydroxymethyl)-1,1-dioxido-2,3-dihydrobenzo[d]isothiazol-5-yl)amino)-1H-pyrazol-5-yl)cyclopentyl isopropylcarbamat

### Step 1:5-bromo-4-fluoro-7-(hydroxymethyl)-2,3-dihydrobenzo[d]isothiazole 1,1-dioxide

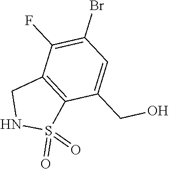 [SMILES: O=S1(=O)NCc2c(F)c(Br)cc(CO)c21]

The titled compound was synthesized in the procedures similar to Example 345. LC-MS (ESI): m/z [M+H]⁺=295.9.

### Step 2: cis-3-(3-((4-fluoro-7-(hydroxymethyl)-1,1-dioxido-2,3-dihydrobenzo[d]isothiazol-5-yl)amino-1H-pyrazol-5-yl)cyclopentyl isopropylcarbamate

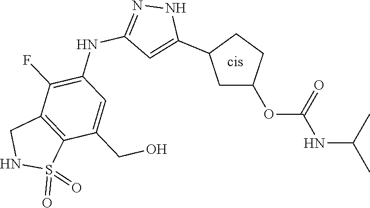 [SMILES: CC(C)NC(=O)OC1CCC(c2cc(Nc3cc(CO)c4c(c3F)CNS4(=O)=O)n[nH]2)C1]

The titled compound was synthesized in the procedures similar to Example 345. ¹H NMR (500 MHz, DMSO-d₆) δ 12.03 (s, 1H), 8.09-7.93 (m, 2H), 7.20 (s, 1H), 6.94 (d, J=5 Hz, 1H), 5.92 (s, 1H), 5.41 (t, J=5 Hz, 1H), 5.05-4.95 (m, 1H), 4.53 (d, J=5 Hz, 2H), 4.38-4.31 (m, 2H), 3.63-3.52 (m, 1H), 3.12-3.01 (m, 1H), 2.47-2.42 (m, 1H), 2.06-1.97 (m, 1H), 1.95-1.84 (m, 1H), 1.79-1.66 (m, 2H), 1.65-1.56 (m, 1H), 1.09-0.96 (m, 6H). LC-MS (ESI): m/z [M+H]⁺=468.2.

### Example 366: cis-3-(3-((4-fluoro-7-(hydroxymethyl)-1,1-dioxido-2,3-dihydrobenzo[d]isothiazol-5-yl)amino)-1H-pyrazol-5-yl)cyclopentyl propylcarbamate

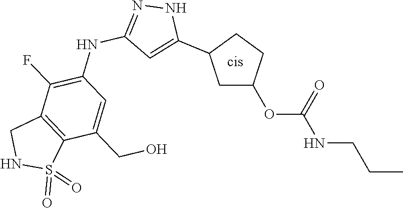 [SMILES: CCCNC(=O)OC1CCC(c2cc(Nc3cc(CO)c4c(c3F)CNS4(=O)=O)n[nH]2)C1]

The titled compound was synthesized in the procedures similar to Example 365. ¹H NMR (500 MHz, DMSO-d₆) δ 12.02 (s, 1H), 8.05-7.94 (m, 2H), 7.19 (s, 1H), 7.04 (t, J=5 Hz, 1H), 5.92 (s, 1H), 5.41 (t, J=5 Hz, 1H), 5.04-4.96 (m, 1H), 4.53 (d, J=5 Hz, 2H), 4.35 (d, J=5 Hz, 2H), 3.12-3.02 (m, 1H), 2.95-2.88 (m, 2H), 2.47-2.41 (m, 1H), 2.07-1.98 (m, 1H), 1.95-1.84 (m, 1H), 1.79-1.66 (m, 2H), 1.65-1.56 (m, 1H), 1.43-1.33 (m, 2H), 0.81 (t, J=5 Hz, 1H). LC-MS (ESI): m/z [M+H]⁺=468.2.

### Example 367: cis-3-(3-((1-cyclopropyl-4-fluoro-2,2-dioxido-1,3-dihydrobenzo[c]isothiazol-5-yl)amino)-1H-pyrazol-5-yl)cyclopentyl(1-methoxypropan-2-yl)carbamate

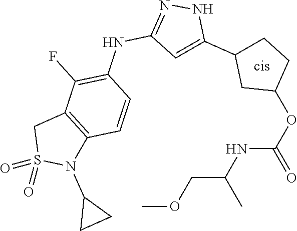 [SMILES: COCC(C)NC(=O)OC1CCC(c2cc(Nc3ccc4c(c3F)CS(=O)(=O)N4C3CC3)n[nH]2)C1]

The titled compound was synthesized in the procedures similar to Example 362. ¹H NMR (500 MHz, DMSO-d₆) δ 11.72 (s, 1H), 8.03 (s, 1H), 7.92 (s, 1H), 6.95 (d, J=7.7 Hz, 1H), 6.83 (d, J=8.7 Hz, 1H), 5.68 (s, 1H), 4.99 (s, 1H), 4.70 (s, 2H), 3.70-3.61 (m, 1H), 3.24-3.20 (m, 4H), 3.14-3.10 (m, 1H), 3.08-2.96 (m, 1H), 2.47-2.40 (m, 1H), 2.05-1.97 (m, 1H), 1.95-1.84 (m, 1H), 1.81-1.66 (m, 3H), 1.65-1.53 (m, 1H), 1.04-0.98 (m, 3H), 0.96-0.91 (m, 2H), 0.91-0.81 (m, 2H). LC-MS (ESI): m/z [M+H]⁺=508.2.

### Example 368: cis-3-(3-((1-cyclopropyl-4-fluoro-2,2-dioxido-1,3-dihydrobenzo[c]isothiazol-5-yl)amino)-1H-pyrazol-5-yl)cyclopentyl(3-methyltetrahydrofuran-3-yl)carbamate

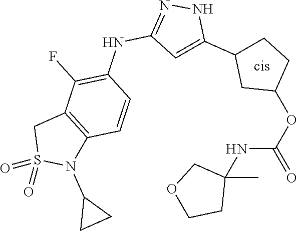 [SMILES: CC1(NC(=O)OC2CCC(c3cc(Nc4ccc5c(c4F)CS(=O)(=O)N5C4CC4)n[nH]3)C2)CCOC1]

The titled compound was synthesized in the procedures similar to Example 362. ¹H NMR (500 MHz, DMSO-d₆) δ 11.73 (s, 1H), 8.02 (s, 1H), 7.93 (s, 1H), 7.23 (s, 1H), 6.83 (d, J=8.7 Hz, 1H), 5.68 (s, 1H), 4.99 (s, 1H), 4.70 (s, 2H), 3.89-3.80 (m, 1H), 3.78-3.69 (m, 2H), 3.43 (d, J=8.6 Hz, 2H), 3.09-2.97 (m, 1H), 2.49-2.38 (m, 1H), 2.21-2.11 (m, 1H), 2.06-1.97 (m, 1H), 1.97-1.85 (m, 1H), 1.80-1.64 (m, 3H), 1.64-1.52 (m, 1H), 1.32 (s, 3H), 0.97-0.91 (m, 2H), 0.90-0.85 (m, 2H). LC-MS (ESI): m/z [M+H]⁺=520.2.

### Example 369: cis-3-(3-((1-methyl-2,2-dioxido-1,3-dihydrobenzo[c]isothiazol-5-yl)amino)-1H-pyrazol-5-yl)cyclopentyl(1-methoxy-2-methylpropan-2-yl)carbamate

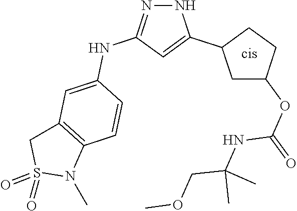 [SMILES: COCC(C)(C)NC(=O)OC1CCC(c2cc(Nc3ccc4c(c3)CS(=O)(=O)N4C)n[nH]2)C1]

The titled compound was synthesized in the procedures similar to Example 324. ¹H NMR (500 MHz, DMSO-d₆) δ 11.62 (s, 1H), 8.22 (s, 1H), 7.42 (s, 1H), 7.26 (d, J=8.0 Hz, 1H), 6.78 (d, J=8.7 Hz, 1H), 6.64 (s, 1H), 5.57 (s, 1H), 4.96 (s, 1H), 4.56 (s, 2H), 3.24 (s, 3H), 3.01 (dd, J=17.2, 8.8 Hz, 1H), 2.95 (s, 3H), 2.48-2.39 (m, 1H), 2.06-1.97 (m, 1H), 1.97-1.85 (m, 1H), 1.80-1.64 (m, 3H), 1.64-1.52 (m, 1H), 1.16 (s, 6H). LC-MS (ESI): m/z [M+H]⁺=478.2.

### Example 370: cis-3-(3-((2,2-dioxido-1,3-dihydrobenzo[c]isothiazol-5-yl)amino)-1H-pyrazol-5-yl)cyclopentyl(1-methoxy-2-methylpropan-2-yl)carbamate

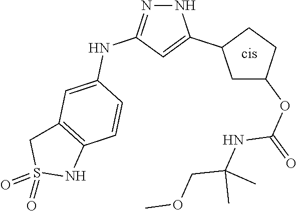 [SMILES: COCC(C)(C)NC(=O)OC1CCC(c2cc(Nc3ccc4c(c3)CS(=O)(=O)N4)n[nH]2)C1]

The titled compound was synthesized in the procedures similar to Example 343. ¹H NMR (500 MHz, DMSO-d₆) δ 11.77 (s, 1H), 9.75 (s, 1H), 8.20 (s, 1H), 7.37 (s, 1H), 7.15 (d, J=8.4 Hz, 1H), 6.71 (d, J=8.6 Hz, 1H), 6.64 (s, 1H), 5.58 (s, 1H), 4.96 (s, 1H), 4.44 (s, 2H), 3.24 (s, 3H), 3.06-2.96 (m, 1H), 2.46-2.40 (m, 1H), 2.05-1.95 (m, 1H), 1.95-1.85 (m, 1H), 1.80-1.65 (m, 3H), 1.62-1.53 (m, 1H), 1.16 (s, 6H). LC-MS (ESI): m/z [M+H]⁺=464.2.

### Example 371: cis-3-(3-((2-cyclopropyl-4-fluoro-1,1-dioxido-2,3-dihydrobenzo[d]isothiazol-5-yl)amino)-1H-pyrazol-5-yl)cyclopentyl(1-methoxy-2-methylpropan-2-yl)carbamate

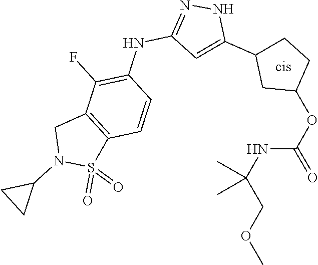 [SMILES: COCC(C)(C)NC(=O)OC1CCC(c2cc(Nc3ccc4c(c3F)CN(C3CC3)S4(=O)=O)n[nH]2)C1]

The titled compound was synthesized in the procedures similar to Example 357. ¹H NMR (500 MHz, DMSO-d₆) δ 12.00 (s, 1H), 8.78 (s, 1H), 8.29 (t, J=5 Hz, 1H), 7.52 (d, J=10 Hz, 1H), 6.63 (s, 1H), 5.83 (s, 1H), 5.02-4.96 (m, 1H), 4.52 (s, 2H), 3.31-3.28 (m, 2H), 3.24 (s, 3H), 3.10-3.01 (m, 1H), 2.48-2.43 (m, 2H), 2.06-1.98 (m, 1H), 1.95-1.85 (m, 1H), 1.77-1.65 (m, 2H), 1.63-1.54 (m, 1H), 1.16 (m, 6H), 0.87-0.82 (m, 2H), 0.76-0.71 (m, 2H). LC-MS (ESI): m/z [M+H]⁺=522.2.

### Example 372: cis-3-(3-((2-cyclopropyl-4-fluoro-1,1-dioxido-2,3-dihydrobenzo[d]isothiazol-5-yl)amino)-1H-pyrazol-5-yl)cyclopentyl(3-methyloxetan-3-yl)carbamate

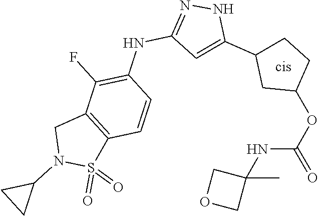 [SMILES: CC1(NC(=O)OC2CCC(c3cc(Nc4ccc5c(c4F)CN(C4CC4)S5(=O)=O)n[nH]3)C2)COC1]

The titled compound was synthesized in the procedures similar to Example 357. ¹H NMR (500 MHz, DMSO-d₆) δ 12.02 (s, 1H), 8.79 (s, 1H), 8.29 (t, J=8.0 Hz, 1H), 7.62 (s, 1H), 7.52 (d, J=8.6 Hz, 1H), 5.84 (s, 1H), 5.10-4.95 (m, 1H), 4.56 (s, 2H), 4.52 (s, 2H), 4.24 (d, J=4.1 Hz, 2H), 3.12-3.01 (m, 1H), 2.54-2.51 (m, 1H), 2.49-2.45 (m 1H), 2.09-2.00 (m, 1H), 1.98-1.85 (m, 1H), 1.81-1.67 (m, 2H), 1.68-1.58 (m, 1H), 1.47 (s, 3H), 0.89-0.80 (m, 2H), 0.80-0.68 (m, 2H). LC-MS (ESI): m/z [M+H]⁺=506.29.

### Example 373: cis-3-(3-((4-fluoro-1,1-dioxido-2,3-dihydrobenzo[d]isothiazol-5-yl)amino)-1H-pyrazol-5-yl)cyclopentyl morpholine-4-carboxylate

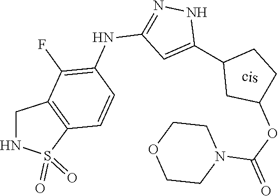 [SMILES: O=C(OC1CCC(c2cc(Nc3ccc4c(c3F)CNS4(=O)=O)n[nH]2)C1)N1CCOCC1]

The titled compound was synthesized in the procedures similar to Example 328. ¹H NMR (500 MHz, DMSO-d₆) δ=12.00 (s, 1H), 8.72 (d, J=1.8, 1H), 8.27 (s, 1H), 7.69 (t, J=5.1, 1H), 7.48 (d, J=8.6, 1H), 5.82 (s, 1H), 5.05 (m, 1H), 4.41 (d, J=5.2, 2H), 3.50 (s, 4H), 3.31-3.25 (m, 4H), 3.12 (m, 1H), 2.47-2.39 (m, 1H), 2.03 (m, 1H), 1.92 (m, 1H), 1.82-1.67 (m, 3H). LC-MS (ESI): m/z [M+H]⁺=466.31.

### Example 374: cis-3-(3-((4-fluoro-2,2-dioxido-1,3-dihydrobenzo[c]isothiazol-5-yl)amino)-1H-pyrazol-5-yl)cyclopentyl(3-methyloxetan-3-yl)carbamate

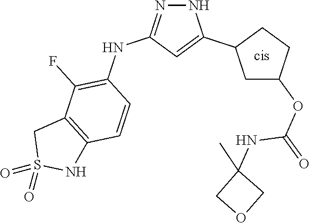 [SMILES: CC1(NC(=O)OC2CCC(c3cc(Nc4ccc5c(c4F)CS(=O)(=O)N5)n[nH]3)C2)COC1]

The titled compound was synthesized in the procedures similar to Example 251. ¹H NMR (500 MHz, DMSO-d₆) δ 11.81 (s, 1H), 10.20 (s, 1H), 7.95-7.85 (m, 2H), 7.62 (s, 1H), 6.59 (d, J=8.6 Hz, 1H), 5.68 (d, J=3.1 Hz, 1H), 5.00 (s, 1H), 4.65-4.45 (m, 4H), 4.24 (s, 2H), 3.08-2.89 (m, 1H), 2.47-2.41 (m, 1H), 2.08-1.97 (m, 1H), 1.92-1.81 (m, 1H), 1.79-1.67 (m, 2H), 1.62-1.53 (m, 1H), 1.47 (s, 3H).

C-MS (ESI): m/z [M+H]⁺=466.2.

### Example 375: cis-3-(3-((4-fluoro-2,2-dioxido-1,3-dihydrobenzo[c]isothiazol-5-yl)amino)-1H-pyrazol-5-yl)cyclopentyl(3-methyltetrahydrofuran-3-yl)carbamate

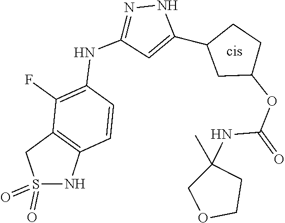 [SMILES: CC1(NC(=O)OC2CCC(c3cc(Nc4ccc5c(c4F)CS(=O)(=O)N5)n[nH]3)C2)CCOC1]

The titled compound was synthesized in the procedures similar to Example 251. ¹H NMR (500 MHz, DMSO-d₆) δ 11.73 (s, 1H), 10.20 (s, 1H), 8.02 (s, 1H), 7.93 (s, 1H), 7.23 (s, 1H), 6.83 (d, J=8.7 Hz, 1H), 5.68 (s, 1H), 4.99 (s, 1H), 4.70 (s, 2H), 3.89-3.80 (m, 1H), 3.78-3.69 (m, 2H), 3.43 (d, J=8.6 Hz, 2H), 3.09-2.97 (m, 1H), 2.49-2.38 (m, 1H), 2.21-2.11 (m, 1H), 2.06-1.97 (m, 1H), 1.97-1.85 (m, 1H), 1.80-1.64 (m, 3H), 1.64-1.52 (m, 1H), 1.32 (s, 3H). LC-MS (ESI): m/z [M+H]⁺=480.2.

### Example 376: cis-3-(3-((4-fluoro-2,2-dioxido-1,3-dihydrobenzo[c]isothiazol-5-yl)amino)-1H-pyrazol-5-yl)cyclopentyl(1-methoxy-2-methylpropan-2-yl)carbamate

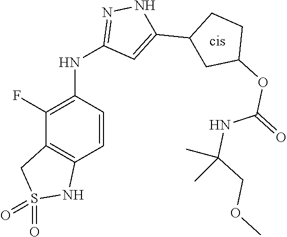 [SMILES: COCC(C)(C)NC(=O)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.66 (s, 1H), 7.90 (s, 1H), 7.82 (s, 1H), 6.63 (s, 1H), 6.55 (t, J=13.0 Hz, 1H), 5.66 (s, 1H), 4.96 (s, 1H), 4.56 (s, 2H), 3.24 (s, 3H), 3.10-3.00 (m, 1H), 2.46-2.37 (m, 1H), 2.04-1.94 (m, 1H), 1.94-1.83 (m, 1H), 1.76-1.64 (m, 2H), 1.62-1.53 (m, 1H), 1.16 (s, 6H). LC-MS (ESI): m/z [M+H]⁺=482.2.

### Example 377: cis-3-(3-((4-fluoro-1-methyl-2,2-dioxido-1,3-dihydrobenzo[c]isothiazol-5-yl)amino)-1H-pyrazol-5-yl)cyclopentyl(1-methoxy-2-methylpropan-2-yl)carbamate

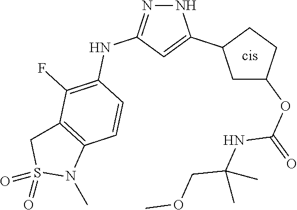 [SMILES: COCC(C)(C)NC(=O)OC1CCC(c2cc(Nc3ccc4c(c3F)CS(=O)(=O)N4C)n[nH]2)C1]

The titled compound was synthesized in the procedures similar to Example 327. ¹H NMR (500 MHz, DMSO-d₆) δ 11.69 (s, 1H), 8.04 (d, J=8.0 Hz, 1H), 7.92 (s, 1H), 6.73-6.59 (m, 2H), 5.68 (s, 1H), 4.96 (s, 1H), 4.72 (s, 2H), 3.31-3.29 (m, 2H), 3.24 (s, 3H), 3.04-2.97 (m, 4H), 2.45-2.42 (m, 1H), 2.04-1.82 (m, 2H), 1.75-1.52 (m, 3H), 1.16 (s, 6H). LC-MS (ESI): m/z [M+H]⁺=496.4.

### Example 378:5-(1-(tert-butyl)-3-((4-fluoro-2-(4-methoxybenzyl)-1,1-dioxido-2,3-dihydrobenzo[d]isothiazol-5-yl)amino)-1H-pyrazol-5-yl)tetrahydrofuran-3-yl isopropylcarbamate

### Step 1: methyl 4,4-dimethoxytetrahydrofuran-2-carboxylate

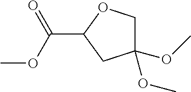

To a solution of ethyl 4-oxotetrahydrofuran-2-carboxylate (20 g, 126.6 mmol) in MeOH (250 mL) were added Trimethoxymethane (20.1 g, 189.9 mmol), H₂SO₄ (0.5 mL) at 70° C. for overnight. The reaction was concentrated under reduced pressure. The resulting solution was quenched with EA. The combined organic phases was washed with NaHCO₃ solution and concentrated under reduced pressure. The crude product (20 g, 83%) as colorless oil. LCMS (ESI) m/z [M+H]⁺=191.

### Step 2:3-(4,4-dimethoxytetrahydrofuran-2-yl)-3-oxopropanenitrile

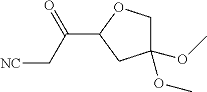

To a solution of acetonitrile (8.04 g, 196 mmol) in dry THF (400 mL) was added n-BuLi (2.5 M) (78.4 mL, 196 mmol), at −78° C. under nitrogen atmosphere for 1 h. The methyl 4,4-dimethoxytetrahydrofuran-2-carboxylate was added in it at −78° C. for 1 h. The reaction was quenched with NH₄Cl solution. The resulting mixture was extracted with EA. The organic layers was concentrated under vacuum. The residue was purified by combi-flash (DCM/EA=54%: 46%) to give the product (13 g, 62%) as light brown oil. LCMS (ESI) m/z [M−H]⁻=198.

### Step 3:1-(tert-butyl)-3-(4,4-dimethoxytetrahydrofuran-2-yl)-1H-pyrazol-5-amine

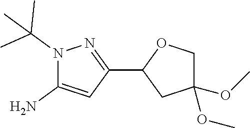 [SMILES: COC1(OC)COC(c2cc(N)n(C(C)(C)C)n2)C1]

To a solution of 3-(4,4-dimethoxytetrahydrofuran-2-yl)-3-oxopropanenitrile (13 g, 65.3 mmol) in MeOH (200 mL) was added tert-butylhydrazine (8.9 g, 71.8 mmol) and TsOH (130 mg) at reflux for 3 h. The residue was purified by combi-flash (CH₃CN: H₂O=78%: 22%) to give the product (1.148 g, 6.7%) as light brown oil. ¹H NMR (500 MHz, DMSO-d₆) δ 5.43-5.35 (m, 1H), 4.82 (s, 2H), 4.65 (dtd, J=9.4, 6.7, 2.5 Hz, 1H), 3.78 (td, J=9.2, 3.5 Hz, 1H), 3.63 (ddd, J=10.6, 9.2, 5.0 Hz, 1H), 3.49-3.40 (m, 3H), 3.17 (d, J=2.6 Hz, 3H), 2.33-2.25 (m, 1H), 2.13-2.01 (m, 1H), 1.50 (s, 14H), 1.26-1.04 (m, 9H). LCMS (ESI) m/z [M+H]⁺=270.

### Step 4:5-((1-(tert-butyl)-5-(4,4-dimethoxytetrahydrofuran-2-yl)-1H-pyrazol-3-yl)amino)-4-fluoro-2-(4-methoxybenzyl)-2,3-dihydrobenzo[d]isothiazole 1,1-dioxide

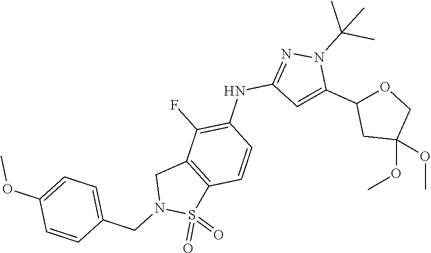

To a mixture of 1-(tert-butyl)-3-(4,4-dimethoxytetrahydrofuran-2-yl)-1H-pyrazol-5-amine (500 mg, 1.86 mmol) and 5-bromo-4-fluoro-2-(4-methoxybenzyl)-2,3-dihydrobenzo[d]isothiazole 1,1-dioxide (715.6 mg, 1.86 mmol) in t-BuOH (20 mL) was added Brettphos-Pd-G3 (62.72 mg, 0.372 mmol), and K₂CO₃ (514.1 mg, 3.72 mmol). The reaction mixture was stirred at 110° C. under N₂ atmosphere for 16 h. The mixture was filtered, and the filtrate was concentrated under reduced pressure. The residue was purified by silica gel column chromatography, eluting with PE/EA (2:1) to afford the product (700 mg, 65.6%). LC-MS (ESI): m/z [M+H]⁺=575.2

### Step 5:5-(1-(tert-butyl)-3-((4-fluoro-2-(4-methoxybenzyl)-1,1-dioxido-2,3-dihydrobenzo[d]isothiazol-5-yl)amino)-1H-pyrazol-5-yl) dihydrofuran-3 (2H)-one

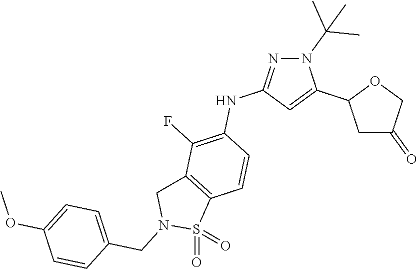 [SMILES: COc1ccc(CN2Cc3c(ccc(Nc4cc(C5CC(=O)CO5)n(C(C)(C)C)n4)c3F)S2(=O)=O)cc1]

A solution of 5-((1-(tert-butyl)-5-(4,4-dimethoxytetrahydrofuran-2-yl)-1H-pyrazol-3-yl)amino)-4-fluoro-2-(4-methoxybenzyl)-2,3-dihydrobenzo[d]isothiazole 1,1-dioxide (700 mg, 1.22 mmol) in THF:H₂O (1:1) (15 mL) was added TsOH (419.9 mg, 2.44 mmol), The reaction mixture was stirred at 60° C. under N₂ atmosphere for 16 h. The mixture was concentrated under reduced pressure. The residue was purified by silica gel column chromatography, eluting with PE/EA (2:1) to afford the product (600 mg, 93.1%). LC-MS (ESI): m/z [M+H]⁺=529.2.

### Step 6:5-((1-(tert-butyl)-5-(4-hydroxytetrahydrofuran-2-yl)-1H-pyrazol-3-yl)amino)-4-fluoro-2-(4-methoxybenzyl)-2,3-dihydrobenzo[d]isothiazole 1,1-dioxide

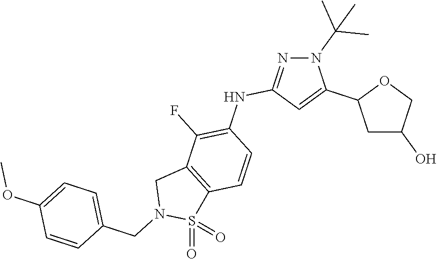 [SMILES: COc1ccc(CN2Cc3c(ccc(Nc4cc(C5CC(O)CO5)n(C(C)(C)C)n4)c3F)S2(=O)=O)cc1]

To a solution of 5-(1-(tert-butyl)-3-((4-fluoro-2-(4-methoxybenzyl)-1,1-dioxido-2,3-dihydrobenzo[d]isothiazol-5-yl)amino)-1H-pyrazol-5-yl) dihydrofuran-3 (2H)-one (600 mg, 1.13 mmol) of MeOH (30 mL) at 0° C. was slowly added NaBH₄ (85.9 mg, 2.27 mmol) and The reaction mixture was stirred at rt under N₂ atmosphere for 2 h. After completion of the reaction, the reaction mixture was dissolved in EtOAc (50 mL). The organic layer was washed with saturated NaCl solution (30 mL), dried over anhydrous MgSO₄, and filtered. The filtrate was concentrated in vacuo and purified by silica gel column chromatography, eluting with PE/EA (1:2) to afford the product to give product (550 mg, 91.3%).

LC-MS (ESI): m/z [M+H]⁺=531.2.

### Step 7:5-(1-(tert-butyl)-3-((4-fluoro-2-(4-methoxybenzyl)-1,1-dioxido-2,3-dihydrobenzo[d]isothiazol-5-yl)amino)-1H-pyrazol-5-yl)tetrahydrofuran-3-yl isopropylcarbamate

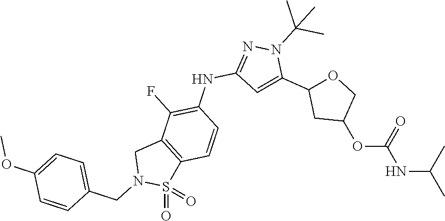 [SMILES: COc1ccc(CN2Cc3c(ccc(Nc4cc(C5CC(OC(=O)NC(C)C)CO5)n(C(C)(C)C)n4)c3F)S2(=O)=O)cc1]

To a solution of 5-((1-(tert-butyl)-5-(4-hydroxytetrahydrofuran-2-yl)-1H-pyrazol-3-yl)amino)-4-fluoro-2-(4-methoxybenzyl)-2,3-dihydrobenzo[d]isothiazole 1,1-dioxide (130 mg, 0.245 mmol) in THF (5 mL) and the solution was cooled to 0° C. and NaH (19.6 mg, 0.49 mmol) was slowly added. After 30 minutes at 0° C., 2-isocyanatopropane (31.3 mg, 0.367 mmol) was added and the mixture was stirred at RT for 1 h. The reaction mixture was dissolved in EtOAc (20 mL). The organic layer was washed with saturated NH₄Cl solution (30 mL), dried over anhydrous MgSO₄, and filtered. The filtrate was concentrated in vacuo to give product (130 mg, 86.2%). LC-MS (ESI): m/z [M+H]⁺=616.3.

### Step 8:5-(3-((4-fluoro-1,1-dioxido-2,3-dihydrobenzo[d]isothiazol-5-yl)amino)-1H-pyrazol-5-yl)tetrahydrofuran-3-yl isopropylcarbamate

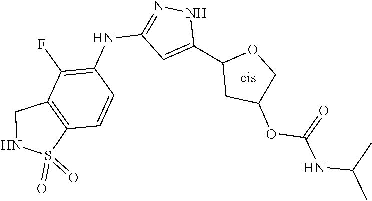 [SMILES: CC(C)NC(=O)OC1COC(c2cc(Nc3ccc4c(c3F)CNS4(=O)=O)n[nH]2)C1]

The titled compound was synthesized in the procedures similar to Example 1. ¹H NMR (500 MHz, DMSO-d₆) δ 12.22 (s, 1H), 8.78 (s, 1H), 8.26 (s, 1H), 7.71 (s, 1H), 7.49 (d, J=8.6 Hz, 1H), 7.10 (d, J=7.6 Hz, 1H), 5.99 (s, 1H), 5.17 (d, J=3.1 Hz, 1H), 4.85 (s, 1H), 4.41 (s, 2H), 3.86 (s, 2H), 3.58 (dd, J=13.3, 6.7 Hz, 1H), 2.77-2.64 (m, 1H), 1.91 (s, 1H), 1.04 (dd, J=6.0, 3.0 Hz, 6H). LC-MS (ESI): m/z [M+H]⁺=440.1.

### Example 379: cis-3-(3-((4-fluoro-2-methyl-1,1-dioxido-2,3-dihydrobenzo[d]isothiazol-5-yl)amino)-1H-pyrazol-5-yl)cyclopentyl(1-methoxy-2-methylpropan-2-yl)carbamate

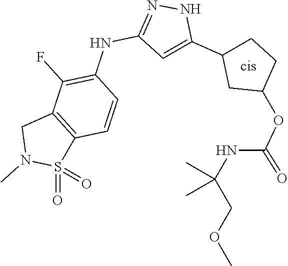 [SMILES: COCC(C)(C)NC(=O)OC1CCC(c2cc(Nc3ccc4c(c3F)CN(C)S4(=O)=O)n[nH]2)C1]

The titled compound was synthesized in the procedures similar to Example 335. ¹H NMR (500 MHz, DMSO-d₆) δ 12.01 (s, 1H), 8.80 (s, 1H), 8.29 (t, J=8.0 Hz, 1H), 7.55 (d, J=8.6 Hz, 1H), 6.66 (s, 1H), 5.84 (s, 1H), 4.97 (s, 1H), 4.43 (s, 2H), 3.30 (s, 2H), 3.24 (s, 3H), 3.11-3.00 (m, 1H), 2.78 (s, 3H), 2.48-2.43 (m, 1H), 2.02 (dd, J=14.4, 6.6 Hz, 1H), 1.89 (qd, J=12.8, 8.1 Hz, 1H), 1.77-1.64 (m, 2H), 1.58 (dt, J=19.1, 9.6 Hz, 1H), 1.16 (s, 6H). LC-MS (ESI): m/z [M+H]⁺=496.2.

### Example 380: cis-3-(3-((2,2-dioxido-1,3-dihydrobenzo[c]isothiazol-5-yl)amino)-1H-pyrazol-5-yl)cyclopentyl cyclobutylcarbamate

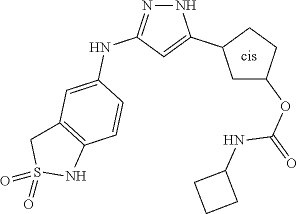 [SMILES: O=C(NC1CCC1)OC1CCC(c2cc(Nc3ccc4c(c3)CS(=O)(=O)N4)n[nH]2)C1]

The titled compound was synthesized in the procedures similar to Example 343 in a racemic form, which was further separated by Chiral Prep-HPLC to give:

Enantiomer 1 (Example 380a, 100% ee); Retention time: 5.749 min. ¹H NMR (500 MHz, DMSO-d₆) δ=11.63 (s, 1H), 9.75 (s, 1H), 8.21 (s, 1H), 7.38 (d, J=7.6, 2H), 7.17 (s, 1H), 6.71 (d, J=8.6, 1H), 5.57 (s, 1H), 4.98 (m, 1H), 4.44 (s, 2H), 3.94 (m, 1H), 3.09-2.97 (m, 1H), 2.44 (m, 1H), 2.09 (m, 2H), 2.00 (m, 1H), 1.94-1.80 (m, 3H), 1.68 (m, 2H), 1.54 (m, 3H). LC-MS (ESI): m/z [M+H]⁺=432.39.

Enantiomer 2 (Example 380b, 100% ee); Retention time: 8.266 min. ¹H NMR (500 MHz, DMSO-d₆) δ=11.63 (s, 1H), 9.75 (s, 1H), 8.21 (s, 1H), 7.38 (d, J=7.5, 2H), 7.16 (d, J=6.7, 1H), 6.71 (d, J=8.6, 1H), 5.57 (s, 1H), 4.98 (m, 1H), 4.44 (s, 2H), 3.94 (m, 1H), 3.13-2.95 (m, 1H), 2.47-2.39 (m, 1H), 2.09 (m, 2H), 2.03-1.95 (m, 1H), 1.94-1.80 (m, 3H), 1.68 (m, 2H), 1.62-1.48 (m, 3H). LC-MS (ESI): m/z [M+H]⁺=432.36;

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=60: 40 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=60: 40 (v/v); Flow Rate: 18 mL/min, Wavelength: UV 270 nm and 320 nm, Prep-HPLC Equipment: Prep-HPLC-Gilson; Column temperature: 25° C.

### Example 381: cis-3-(3-((2,2-dioxido-1,3-dihydrobenzo[c]isothiazol-5-yl)amino)-1H-pyrazol-5-yl)cyclopentyl cyclopentylcarbamate

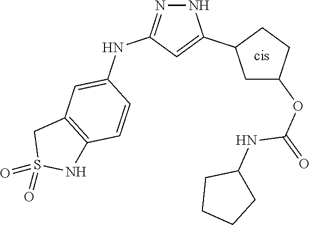 [SMILES: O=C(NC1CCCC1)OC1CCC(c2cc(Nc3ccc4c(c3)CS(=O)(=O)N4)n[nH]2)C1]

The titled compound was synthesized in the procedures similar to Example 343 in a racemic form, which was further separated by Chiral Prep-HPLC to give:

Enantiomer 1 (Example 381a, 100% ee); Retention time: 5.876 min. ¹H NMR (500 MHz, DMSO-d₆) δ=11.62 (s, 1H), 9.75 (s, 1H), 8.21 (s, 1H), 7.38 (s, 1H), 7.15 (s, 1H), 7.07 (d, J=7.2, 1H), 6.71 (d, J=8.6, 1H), 5.57 (s, 1H), 4.99 (m, 1H), 4.44 (s, 2H), 3.76 (m, 1H), 3.13-2.94 (m, 1H), 2.48-2.40 (m, 1H), 2.00 (m, 1H), 1.89 (m, 1H), 1.79-1.68 (m, 4H), 1.59 (m, 3H), 1.45 (m, 2H), 1.36 (m, 2H).

LC-MS (ESI): m/z [M+H]⁺=446.34.

Enantiomer 2 (Example 381b, 100% ee); Retention time: 9.083 min. ¹H NMR (500 MHz, DMSO-d₆) δ=11.62 (s, 1H), 9.75 (s, 1H), 8.20 (s, 1H), 7.38 (s, 1H), 7.16 (d, J=8.2, 1H), 7.07 (d, J=7.2, 1H), 6.70 (d, J=8.6, 1H), 5.57 (s, 1H), 4.98 (m, 1H), 4.44 (s, 2H), 3.76 (mm, 1H), 3.08-2.92 (m, 1H), 2.45 (m, 1H), 2.00 (m, 1H), 1.90 (m, 1H), 1.69 (m, 4H), 1.59 (m, 3H), 1.45 (m, 2H), 1.36 (m, 2H).

LC-MS (ESI): m/z [M+H]⁺=446.38.

Chiral analytical method: Column: CHIRALPAK IE 4.6*250 mm 5 μm; Mobile phase: A for MtBE (0.1% 2M NH₃ MeOH) and B for MeOH: DCM=50:50 (v/v); Gradient: Mobile Phase A: Mobile Phase B=60: 40 (v/v); HPLC Equipment: HPLC-Agilent; Column temperature: 35° C.

Chiral Prep-HPLC Condition: CHIRALPAK IE 20*250 mm 5 μm; Mobile phase: A for MtBE and B for MeOH: DCM=50:50 (0.1% 2M NH₃ MeOH); Gradient: Mobile Phase A: Mobile Phase B=60: 40 (v/v); Flow Rate: 18 mL/min, Wavelength: UV 270 nm and 330 nm, Prep-HPLC Equipment: Prep-HPLC-Gilson; Column temperature: 25° C.

### Example 382: cis-3-(3-((2,2-dioxido-1,3-dihydrobenzo[c]isothiazol-5-yl)amino)-1H-pyrazol-5-yl)cyclopentyl morpholine-4-carboxylate

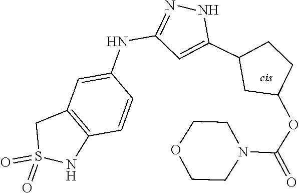 [SMILES: O=C(OC1CCC(c2cc(Nc3ccc4c(c3)CS(=O)(=O)N4)n[nH]2)C1)N1CCOCC1]

The titled compound was synthesized in the procedures similar to Example 343. ¹H NMR (500 MHz, DMSO-d₆) δ 11.75 (s, 1H), 9.76 (s, 1H), 8.22 (s, 1H), 7.36 (s, 1H), 7.15 (d, J=8.3 Hz, 1H), 6.71 (d, J=8.6 Hz, 1H), 5.58 (s, 1H), 5.10-4.90 (m, 1H), 4.44 (s, 2H), 3.50 (s, 4H), 3.30-3.26 (m, 4H), 3.12-3.02 (m, 1H), 2.46-2.37 (m, 1H), 2.04-1.94 (m, 1H), 1.94-1.83 (m, 1H), 1.76-1.64 (m, 2H), 1.62-1.53 (m, 1H). LC-MS (ESI): m/z [M+H]⁺=448.2.

### Example 383: cis-3-(3-((4-fluoro-2,2-dioxido-1,3-dihydrobenzo[c]isothiazol-5-yl)amino)-1H-pyrazol-5-yl)cyclopentyl((S)-sec-butyl)carbamate

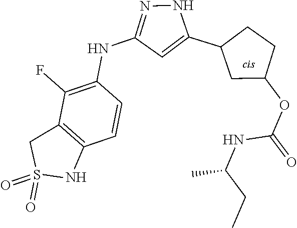 [SMILES: CC[C@H](C)NC(=O)OC1CCC(c2cc(Nc3ccc4c(c3F)CS(=O)(=O)N4)n[nH]2)C1]

The titled compound was synthesized in the procedures similar to Example 251 in a racemic form, which was further separated by Chiral Prep-HPLC to give:

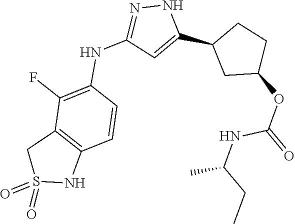 [SMILES: CC[C@H](C)NC(=O)O[C@@H]1CC[C@H](c2cc(Nc3ccc4c(c3F)CS(=O)(=O)N4)n[nH]2)C1]

Enantiomer 1 (Example 383a, (1R,3S)-3-(3-((4-fluoro-2,2-dioxido-1,3-dihydrobenzo[c]isothiazol-5-yl)amino)-1H-pyrazol-5-yl)cyclopentyl((S)-sec-butyl)carbamate, 100% ee); Retention time: 5.245 min. ¹H NMR (500 MHz, DMSO-d₆) δ 11.66 (s, 1H), 10.21 (s, 1H), 7.92 (s, 1H), 7.81 (s, 1H), 6.88 (d, J=7.9 Hz, 1H), 5.66 (s, 1H), 4.98 (s, 1H), 4.56 (s, 2H), 3.47-3.35 (m, 1H), 3.11-2.91 (m, 1H), 2.47-2.39 (m, 1H), 2.06-1.95 (m, 1H), 1.94-1.83 (m, 1H), 1.76-1.64 (m, 2H), 1.62-1.53 (m, 1H), 1.41-1.30 (m, 2H), 1.00 (d, J=6.5 Hz, 3H), 0.80 (t, J=7.4 Hz, 3H). LC-MS (ESI): m/z [M+H]⁺=452.2.

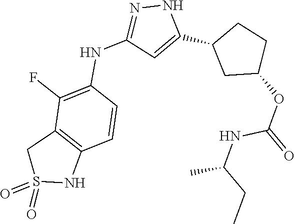 [SMILES: CC[C@H](C)NC(=O)O[C@H]1CC[C@@H](c2cc(Nc3ccc4c(c3F)CS(=O)(=O)N4)n[nH]2)C1]

Enantiomer 2 (Example 383b, (1S,3R)-3-(3-((4-fluoro-2,2-dioxido-1,3-dihydrobenzo[c]isothiazol-5-yl)amino)-1H-pyrazol-5-yl)cyclopentyl((S)-sec-butyl)carbamate, 100% ee); Retention time: 9.373 min. ¹H NMR (500 MHz, DMSO-d₆) δ 11.66 (s, 1H), 10.21 (s, 1H), 7.92 (s, 1H), 7.81 (s, 1H), 6.88 (d, J=7.9 Hz, 1H), 5.66 (s, 1H), 4.98 (s, 1H), 4.56 (s, 2H), 3.47-3.35 (m, 1H), 3.11-2.91 (m, 1H), 2.47-2.39 (m, 1H), 2.06-1.95 (m, 1H), 1.94-1.83 (m, 1H), 1.76-1.64 (m, 2H), 1.62-1.53 (m, 1H), 1.41-1.30 (m, 2H), 1.00 (d, J=6.5 Hz, 3H), 0.80 (t, J=7.4 Hz, 3H). LC-MS (ESI): m/z [M+H]⁺=452.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=60: 40 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 384: cis-3-(3-((3-methyl-2,2-dioxido-1,3-dihydrobenzo[c]isothiazol-5-yl)amino)-1H-pyrazol-5-yl)cyclopentyl isopropylcarbamate

### Step 1:5-bromo-1-(4-methoxybenzyl)-3-methyl-1,3-dihydrobenzo[c]isothiazole 2,2-dioxide

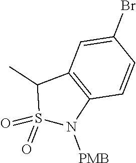

To a solution of 5-bromo-1-(4-methoxybenzyl)-1,3-dihydrobenzo[c]isothiazole 2,2-dioxide (367 mg, 1 mmol) in DMF was added Cs₂CO₃ (489 mg, 2 mmol) and iodomethane (170 mg, 1.2 mmol). The resulting solution was stirred at room temperature for 12 h. The solvent was concentrated, and the residue was purified by silica gel column chromatography, eluting with EA/PE (0-20%) to afford the product (84 mg, 22%).

### Step 2: cis-3-(3-((1-(4-methoxybenzyl)-3-methyl-2,2-dioxido-1,3-dihydrobenzo[c]isothiazol-5-yl)amino-1H-pyrazol-5-yl)cyclopentyl isopropylcarbamate

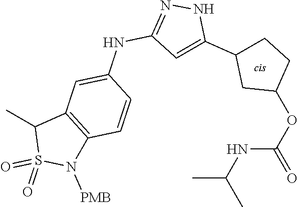

The titled compound was synthesized in the procedures similar to Example 34. LC-MS (ESI): m/z [M+H]⁺=554.2.

### Step 3: cis-3-(3-((3-methyl-2,2-dioxido-1,3-dihydrobenzo[c]isothiazol-5-yl)amino)-1H-pyrazol-5-yl)cyclopentyl isopropylcarbamate

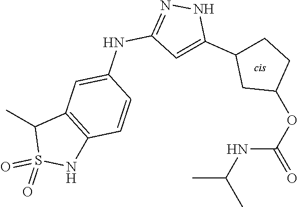 [SMILES: CC(C)NC(=O)OC1CCC(c2cc(Nc3ccc4c(c3)C(C)S(=O)(=O)N4)n[nH]2)C1]

The titled compound was synthesized in the procedures similar to Example 34 in a racemic form, which was separated by Chiral Prep-HPLC to give:

Enantiomer 1 (Example 384a, 100% ee); Retention time: 5.925 min. ¹H NMR (500 MHz, DMSO-d₆) δ 11.68 (s, 1H), 9.68 (s, 1H), 8.22 (s, 1H), 7.16 (d, J=7.1 Hz, 1H), 6.94 (d, J=7.7 Hz, 1H), 6.73 (d, J=8.6 Hz, 1H), 5.57 (s, 1H), 4.98 (s, 1H), 4.39 (q, J=6.9 Hz, 1H), 3.68-3.46 (m, 3H), 2.48-2.43 (m, 1H), 2.00 (dd, J=16.2, 8.4 Hz, 1H), 1.94-1.82 (m, 1H), 1.69 (dd, J=14.0, 7.3 Hz, 2H), 1.63-1.53 (m, 1H), 1.45 (d, J=7.0 Hz, 3H), 1.03 (d, J=6.3 Hz, 6H). LC-MS (ESI): m/z [M+H]⁺=434.2.

Enantiomer 2 (Example 384b, 97.1% ee); Retention time: 6.486 min. ¹H NMR (500 MHz, DMSO-d₆) δ 11.68 (s, 1H), 9.68 (s, 1H), 8.22 (s, 1H), 7.16 (d, J=7.1 Hz, 1H), 6.94 (d, J=7.7 Hz, 1H), 6.73 (d, J=8.6 Hz, 1H), 5.57 (s, 1H), 4.98 (s, 1H), 4.39 (q, J=6.9 Hz, 1H), 3.68-3.46 (m, 3H), 2.48-2.43 (m, 1H), 2.00 (dd, J=16.2, 8.4 Hz, 1H), 1.94-1.82 (m, 1H), 1.69 (dd, J=14.0, 7.3 Hz, 2H), 1.63-1.53 (m, 1H), 1.45 (d, J=7.0 Hz, 3H), 1.03 (d, J=6.3 Hz, 6H). LC-MS (ESI): m/z [M+H]⁺=434.2.

Enantiomer 3 (Example 384c, 100% ee); Retention time: 8.091 min. ¹H NMR (500 MHz, DMSO-d₆) δ 11.68 (s, 1H), 9.68 (s, 1H), 8.22 (s, 1H), 7.16 (d, J=7.1 Hz, 1H), 6.94 (d, J=7.7 Hz, 1H), 6.73 (d, J=8.6 Hz, 1H), 5.57 (s, 1H), 4.98 (s, 1H), 4.39 (q, J=6.9 Hz, 1H), 3.68-3.46 (m, 3H), 2.48-2.43 (m, 1H), 2.00 (dd, J=16.2, 8.4 Hz, 1H), 1.94-1.82 (m, 1H), 1.69 (dd, J=14.0, 7.3 Hz, 2H), 1.63-1.53 (m, 1H), 1.45 (d, J=7.0 Hz, 3H), 1.03 (d, J=6.3 Hz, 6H).LC-MS (ESI): m/z [M+H]⁺=434.2.

Enantiomer 4 (Example 384d, 100% ee); Retention time: 12.776 min. ¹H NMR (500 MHz, DMSO-d₆) δ 11.68 (s, 1H), 9.68 (s, 1H), 8.22 (s, 1H), 7.16 (d, J=7.1 Hz, 1H), 6.94 (d, J=7.7 Hz, 1H), 6.73 (d, J=8.6 Hz, 1H), 5.57 (s, 1H), 4.98 (s, 1H), 4.39 (q, J=6.9 Hz, 1H), 3.68-3.46 (m, 3H), 2.48-2.43 (m, 1H), 2.00 (dd, J=16.2, 8.4 Hz, 1H), 1.94-1.82 (m, 1H), 1.69 (dd, J=14.0, 7.3 Hz, 2H), 1.63-1.53 (m, 1H), 1.45 (d, J=7.0 Hz, 3H), 1.03 (d, J=6.3 Hz, 6H).LC-MS (ESI): m/z [M+H]⁺=434.2.

Chiral analytical method: Column: I-Cellulose-5, 4.6 mm×250 mm, 5 μm; Mobile phase: A for Hexane and B for EtOH (0.1% 2M NH₃ MeOH); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: Column: I-Cellulose-5, 21.2 mm×250 mm, 5 μm; Mobile phase: A for Hexane and B for EtOH (0.2% 2M NH₃ MeOH); Gradient: Mobile Phase A: Mobile Phase B=60: 40 (v/v); Flow Rate: 20 mL/min, Wave Length: UV 200 nm and 270 nm, Prep-HPLC Equipment: Prep-HPLC-Gilson, Back pressure: 100 bar; Column temperature: 25° C.

### Example 385: cis-3-(3-((4-fluoro-1,1-dioxido-2,3-dihydrobenzo[d]isothiazol-5-yl)amino)-1H-pyrazol-5-yl)cyclopentyl(1-(hydroxymethyl)cyclobutyl)carbamate

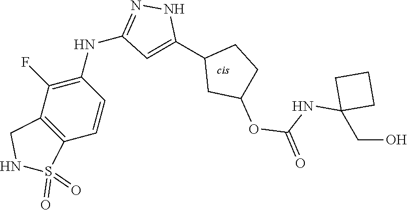 [SMILES: O=C(NC1(CO)CCC1)OC1CCC(c2cc(Nc3ccc4c(c3F)CNS4(=O)=O)n[nH]2)C1]

The titled compound was synthesized in the procedures similar to example 34. ¹H NMR (500 MHz, DMSO-d₆) δ 11.98 (s, 1H), 8.71 (s, 1H), 8.26 (s, 1H), 7.69 (t, J=5.0 Hz, 1H), 7.48 (d, J=8.6 Hz, 1H), 6.96 (s, 1H), 5.84 (s, 1H), 5.10-4.90 (m, 1H), 4.70 (t, J=5.8 Hz, 1H), 4.41 (d, J=5.1 Hz, 2H), 3.45 (d, J=4.9 Hz, 2H), 3.10-3.01 (m, 1H), 2.48-2.41 (m, 1H), 2.15-2.08 (m, 1H), 2.05-1.86 (m, 5H), 1.80-1.68 (m, 3H), 1.67-1.56 (m, 2H). LC-MS (ESI): m/z [M+H]⁺=480.33.

### Example 386: cis-3-(3-((1-methyl-2,2-dioxido-1,3-dihydrobenzo[c]isothiazol-5-yl)amino)-1H-pyrazol-5-yl)cyclopentyl(1-(hydroxymethyl)cyclobutyl)carbamate

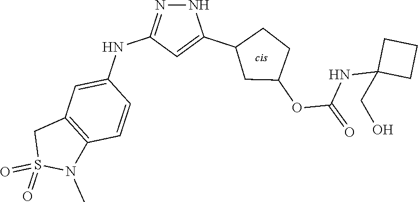 [SMILES: CN1c2ccc(Nc3cc(C4CCC(OC(=O)NC5(CO)CCC5)C4)[nH]n3)cc2CS1(=O)=O]

The titled compound was synthesized in the procedures similar to example 34. ¹H NMR (500 MHz, DMSO-d₆) δ 11.64 (s, 1H), 8.22 (s, 1H), 7.40 (s, 1H), 7.25 (d, J=8.2 Hz, 1H), 6.96 (s, 1H), 6.78 (d, J=8.7 Hz, 1H), 5.58 (s, 1H), 5.06-4.93 (m, 1H), 4.70 (t, J=5.8 Hz, 1H), 4.56 (s, 2H), 3.45 (s, 2H), 3.06-2.99 (m, 1H), 2.95 (s, 3H), 2.48-2.41 (m, 1H), 2.18-2.05 (m, 2H), 2.02-1.93 (m, 3H), 1.92-1.83 (m, 1H), 1.78-1.57 (m, 5H). LC-MS (ESI): m/z [M+H]⁺=476.36.

### Example 387: cis-3-(3-((4-fluoro-2,2-dioxido-1,3-dihydrobenzo[c]isothiazol-5-yl)amino)-1H-pyrazol-5-yl)cyclopentyl propylcarbamate

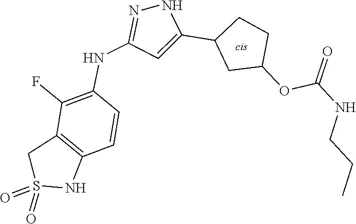 [SMILES: CCCNC(=O)OC1CCC(c2cc(Nc3ccc4c(c3F)CS(=O)(=O)N4)n[nH]2)C1]

The titled compound was synthesized in the procedures similar to Example 251 in a racemic form, which was further separated by Chiral Prep-HPLC to give:

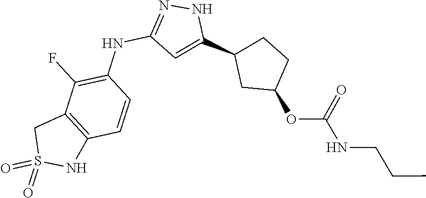 [SMILES: CCCNC(=O)O[C@@H]1CC[C@H](c2cc(Nc3ccc4c(c3F)CS(=O)(=O)N4)n[nH]2)C1]

Enantiomer 1 (Example 387a, (1R,3S)-3-(3-((4-fluoro-2,2-dioxido-1,3-dihydrobenzo[c]isothiazol-5-yl)amino)-1H-pyrazol-5-yl)cyclopentyl propylcarbamate, 100% ee); Retention time: 4.56 min. ¹H NMR (500 MHz, DMSO-d₆) δ 11.69 (s, 1H), 10.21 (s, 1H), 7.95 (s, 1H), 7.87 (s, 1H), 7.05 (t, J=5.6 Hz, 1H), 6.59 (d, J=8.6 Hz, 1H), 5.67 (s, 1H), 4.99 (s, 1H), 4.59 (s, 2H), 3.08-2.98 (m, 1H), 2.91 (dd, J=13.1, 6.6 Hz, 2H), 2.48-2.42 (m, 1H), 2.05-1.85 (m, 2H), 1.76-1.55 (m, 3H), 1.38 (dd, J=14.4, 7.2 Hz, 2H), 0.81 (t, J=7.4 Hz, 3H). LC-MS (ESI): m/z [M+H]⁺=438.3.

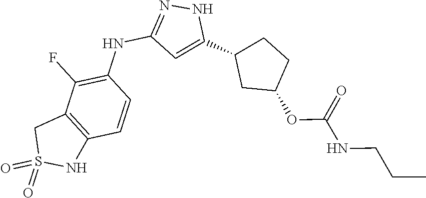 [SMILES: CCCNC(=O)O[C@H]1CC[C@@H](c2cc(Nc3ccc4c(c3F)CS(=O)(=O)N4)n[nH]2)C1]

Enantiomer 2 (Example 387b, (1S,3R)-3-(3-((4-fluoro-2,2-dioxido-1,3-dihydrobenzo[c]isothiazol-5-yl)amino)-1H-pyrazol-5-yl)cyclopentyl propylcarbamate, 100% ee); Retention time: 6.163 min. ¹H NMR (500 MHz, DMSO-d₆) δ 11.69 (s, 1H), 10.21 (s, 1H), 7.95 (s, 1H), 7.87 (s, 1H), 7.05 (t, J=5.6 Hz, 1H), 6.59 (d, J=8.6 Hz, 1H), 5.67 (s, 1H), 4.99 (s, 1H), 4.59 (s, 2H), 3.08-2.98 (m, 1H), 2.91 (dd, J=13.1, 6.6 Hz, 2H), 2.48-2.42 (m, 1H), 2.05-1.85 (m, 2H), 1.76-1.55 (m, 3H), 1.38 (dd, J=14.4, 7.2 Hz, 2H), 0.81 (t, J=7.4 Hz, 3H). LC-MS (ESI): m/z [M+H]⁺=438.3.

Chiral analytical method: Column: CHIRALPAK IE 4.6 mm×250 mm, 5 μm; Mobile phase: A for MTBE (0.1% 2M NH₃ MeOH) and B for MEOH/DCM (50/50); Gradient: Mobile Phase A: Mobile Phase B=40: 60 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MEOH/DCM (50/50); Gradient: Mobile Phase A: Mobile Phase B=40: 60 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 388: cis-3-(3-((4-fluoro-2,2-dioxido-1,3-dihydrobenzo[c]isothiazol-5-yl)amino)-1H-pyrazol-5-yl)cyclopentyl(1-methylcyclopropyl)carbamate

### Step 1: S-(2-chloro-6-fluorobenzyl)ethanethioate

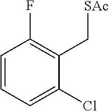

To the solution of 1-chloro-2-(chloromethyl)-3-fluorobenzene (100.0 g, 561.8 mmol, leq) in DMF (1.0 L), was added Potassium thioacetate (128.3 g, 1123.6 mmol, 2.0 eq) at 0° C. Then the reaction was warmed to room temperature and stirred for 1 h. Diluted with water (1 L), extracted with EtOAc (600 mL×3), washed with brine (600 mL×5), dried over Na₂SO₄, concentrated to give product (120.0 g, yield 97.8%). ¹H NMR (300 MHz, DMSO-d₆) δ 7.42-7.33 (m, 2H), 7.27-7.21 (m, 1H), 4.26 (s, 2H), 2.36 (s, 3H).

### Step 2: (2-chloro-6-fluorophenyl) methanesulfonyl chloride

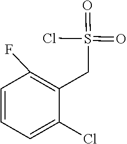 [SMILES: O=S(=O)(Cl)Cc1c(F)cccc1Cl]

To the solution of NCS (244.3 g, 1829.2 mmol, 4.0 eq) in 2N HCl (150 mL) and MeCN (500 mL), a solution of S-(2-chloro-6-fluorobenzyl)ethanethioate (100.0 g, 457.2 mmol, 1.0 eq) in MeCN (500 mL) was added dropwise below 20° C. The mixture was stirred at room temperature for 2 h. Concentrated and diluted with water (200 mL), extracted with EtOAc (300 mL×3), washed with brine (300 mL×2), dried over Na₂SO₄ and concentrated to give product (130.0 g, crude).

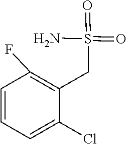 [SMILES: NS(=O)(=O)Cc1c(F)cccc1Cl]

### Step 3: (2-chloro-6-fluorophenyl) methanesulfonamide

Dissolved the (2-chloro-6-fluorophenyl) methanesulfonyl chloride (130.0 g, 534.8 mmol, 1.0 eq) in THF (1.3 L), the mixture was added dropwise to Ammonium hydroxide (28%) (240 mL) at 0° C., then the mixture was stirred at room temperature for 2 h. Then diluted with water (600 mL), extracted with EtOAc (500 mL×3), dried over Na₂SO₄, concentrated to get residue. The residue was triturated with MeCN (200 mL), filtered and the cake was dried under vacuum to give product (72.0 g, yield 68.9% over 2 steps). ¹H NMR (300 MHz, DMSO-d₆) δ 7.48-7.37 (m, 2H), 7.30-7.25 (m, 1H), 7.19 (brs, 2H), 4.48 (s, 2H). LC-MS (ESI): m/z [M+NH_{4]}⁺=241.0.

### Step 4:4-fluoro-1,3-dihydrobenzo[c]isothiazole 2,2-dioxide

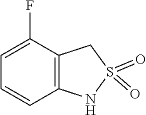 [SMILES: O=S1(=O)Cc2c(F)cccc2N1]

Under N2 atmosphere, to the solution of (2-chloro-6-fluorophenyl) methanesulfonamide (80.0 g, 357.7 mmol, 1.0 eq) in DMF (800 mL), was added CuI (20.4 g, 107.3 mmol, 0.3 eq), K₃PO₄ (379.6 g, 1788.5 mmol, 4.0 eq) and Sarcosine (159.3 g, 1788 mmol, 5.0 eq). The mixture was stirred at 100° C. for 8h. Cooled and diluted with water (800 mL), adjusted pH to 2 with HCl (2N), extracted with EtOAc (800 mL×5), washed with brine (800 mL×5), dried over Na₂SO₄, concentrated to get a residue. The residue was triturated with DCM (300 mL), filtered and the cake was dried under vacuum to give product (52.0 g, yield 77.7%). ¹H NMR (400 MHz, CDCl₃) δ 10.88 (brs, 1H), 7.34-7.27 (m, 1H), 6.83 (t, J=8.7 Hz, 1H), 6.67 (d, J=8.1 Hz, 1H), 4.63 (s, 2H). LC-MS (ESI): m/z [M−H]—=186.0.

### Step 5:5-bromo-4-fluoro-1,3-dihydrobenzo[c]isothiazole 2,2-dioxide

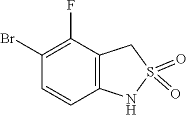 [SMILES: O=S1(=O)Cc2c(ccc(Br)c2F)N1]

Under N2 atmosphere, to a solution of 4-fluoro-1,3-dihydrobenzo[c]isothiazole 2,2-dioxide (40.0 g, 213.7 mmol, 1.0 eq) in AcOH (200 mL), was added dropwise a solution of Br₂ (37.1 g, 235.0 mmol, 1.1 eq) in AcOH (200 mL). Then the mixture was stirred at 15° C. for 3 h. Quenched with 10% Na₂S₂O₃ (400 mL), and ice water (400 mL) was added, the mixture was filtered and the cake was washed with ice water (200 mL×2), the cake was triturated with MeCN (100 mL), and DCM (100 mL), filtered and the cake was dried under vacuum to give 5-bromo-4-fluoro-1,3-dihydrobenzo[c]isothiazole 2,2-dioxide (29.0 g, yield 51.0%). ¹H NMR (400 MHz, DMSO-d₆): δ 11.08 (brs, 1H), 7.58 (t, J=8.0 Hz, 1H), 6.65 (d, J=8.8 Hz, 1H), 4.74 (s, 2H). LC-MS (ESI): m/z [M+H]⁺=265.9.

### Step 6:5-bromo-4-fluoro-1-(4-methoxybenzyl)-1,3-dihydrobenzo[c]isothiazole 2,2-dioxide

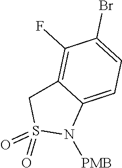

To a solution of 5-bromo-4-fluoro-1,3-dihydrobenzo[c]isothiazole 2,2-dioxide (500 mg, 1.89 mmol) in DMF (10 mL) was added K₂CO₃ (522 mg, 3.78 mmol) and 4-methoxybenzylchloride (444 mg, 2.8 mmol) at 0° C. The result mixture was stirred at room temperature for 2 h. The solution was poured into 20 mL water and extracted with EA. EA layers were combined, dried and concentrated under vacuum. The residue was applied to a silica gel column chromatography, eluting with PE/EtOAc (4:1) to afford the product (512 mg, 70%), LC-MS (ESI): m/z [M+Na]⁺=408.2.

### Step 7: cis-benzyl(1-(tert-butyl)-3-(3-(((4-nitrophenoxy) carbonyl)oxy) cyclopentyl)-1H-pyrazol-5-yl)carbamate

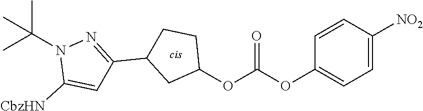 [SMILES: CC(C)(C)n1nc(C2CCC(OC(=O)Oc3ccc([N+](=O)[O-])cc3)C2)cc1NC(=O)OCc1ccccc1]

To a solution of cis-benzyl(1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 8: cis-benzyl(5-(3-(((4-nitrophenoxy) carbonyl)oxy) cyclopentyl)-1H-pyrazol-3-yl)carbamate

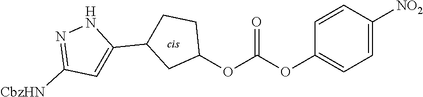 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl(1-(tert-butyl)-3-(3-(((4-nitrophenoxy) carbonyl)oxy) cyclopentyl)-1H-pyrazol-5-yl)carbamate (30 g, 57.5 mmol) was added 100 mL formic acid. The resulting mixture was stirred at 75° C. for over night. The solvent was removed under vacuum to yield 26 g of crude product, which was directly used for the next step without further purification. LC-MS (ESI): m/z [M+H]⁺=467.2.

### Step 9: cis-3-(3-(((benzyloxy) carbonyl)amino)-1H-pyrazol-5-yl)cyclopentyl(1-methylcyclopropyl)carbamate

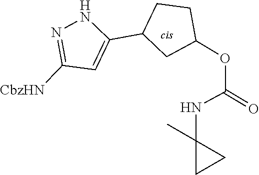 [SMILES: CC1(NC(=O)OC2CCC(c3cc(NC(=O)OCc4ccccc4)n[nH]3)C2)CC1]

To a solution of cis-benzyl(5-(3-(((4-nitrophenoxy) carbonyl)oxy) cyclopentyl)-1H-pyrazol-3-yl)carbamate (10 g, 21.5 mmol) in 100 mL THF was added 1-methylcyclopropylamine hydrochloride (11.5 g, 107 mmol) and DIEA (27.6 g, 214 mmol). The resulting mixture was stirred at room temperature for 12 h. The resulting mixture was concentrated under reduced pressure and dissolved in EA (200 mL). The mixture was washed with water (200 mL×3), dried with Na₂SO₄, and concentrated. The residue was triturated with MTBE and filtered to afford the product (3.6 g, 42% yield). LC-MS (ESI): m/z [M+H]⁺=399.3.

### Step 10: cis-3-(3-amino-1H-pyrazol-5-yl)cyclopentyl(1-methylcyclopropyl)carbamate

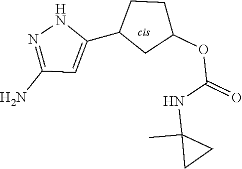 [SMILES: CC1(NC(=O)OC2CCC(c3cc(N)n[nH]3)C2)CC1]

To a solution of cis-3-(3-(((benzyloxy) carbonyl)amino)-1H-pyrazol-5-yl)cyclopentyl(1-methylcyclopropyl)carbamate (3.6 g, 9 mmol) in THF (100 mL) was added Pd/C (10%, wet, 2 g). The suspension was degassed and purged with hydrogen 3 times. The resulting mixture was stirred at room temperature under a hydrogen balloon for 3 h. The mixture was filtered, and the filter cake was washed with EA (200 mL×3). The filtrate was concentrated under reduced pressure to afford the product (2.2 g, 92% yield). LC-MS (ESI): m/z [M+H]⁺=265.3.

### Step 11: cis-3-(3-((4-fluoro-1-(4-methoxybenzyl)-2,2-dioxido-1,3-dihydrobenzo[c]isothiazol-5-yl)amino)-1H-pyrazol-5-yl)cyclopentyl(1-methylcyclopropyl)carbamate

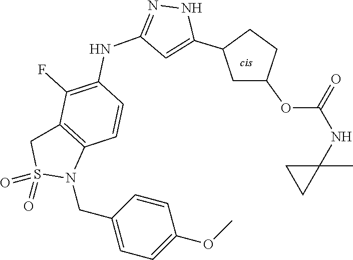 [SMILES: COc1ccc(CN2c3ccc(Nc4cc(C5CCC(OC(=O)NC6(C)CC6)C5)[nH]n4)c(F)c3CS2(=O)=O)cc1]

A mixture of 5-bromo-4-fluoro-1-(4-methoxybenzyl)-1,3-dihydrobenzo[c]isothiazole 2,2-dioxide (190 mg, 0.49 mmol), cis-3-(3-amino-1H-pyrazol-5-yl)cyclopentyl(1-methylcyclopropyl)carbamate (130 mg, 0.49 mmol), Brettphos Pd G3 (45 mg, 0.05 mmol) and K₂CO₃ (207 mg, 1.5 mmol) in t-BuOH (20 mL) was stirred at 110° C. for 16 h under nitrogen atmosphere. The mixture was concentrated under vacuum. The residue was purified by silica gel column chromatography, eluting with EA/PE (2:1~10:1) to afford the product (100 mg, 35.8%). LC-MS (ESI): m/z [M+H]⁺=570.3.

### Step 12: cis-3-(3-((4-fluoro-2,2-dioxido-1,3-dihydrobenzo[c]isothiazol-5-yl)amino)-1H-pyrazol-5-yl)cyclopentyl(1-methylcyclopropyl)carbamate

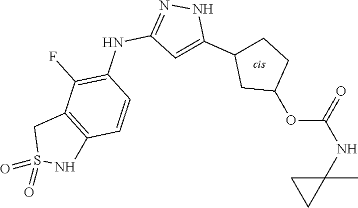 [SMILES: CC1(NC(=O)OC2CCC(c3cc(Nc4ccc5c(c4F)CS(=O)(=O)N5)n[nH]3)C2)CC1]

cis-3-(3-((4-fluoro-1-(4-methoxybenzyl)-2,2-dioxido-1,3-dihydrobenzo[c]isothiazol-5-yl)amino)-1H-pyrazol-5-yl)cyclopentyl(1-methylcyclopropyl)carbamate (200 mg, 0.36 mmol) was dissolved in DCM/TFA (1:1, 20 mL). The resulting solution was stirred at room temperature for 12 h. The solvent was concentrated and the residue was purified by silica gel column chromatography, eluting with EA/PE (0-100%) and further purified by Prep-HPLC to afford the product in a racemic form, which was further separated by Chiral Prep-HPLC to give:

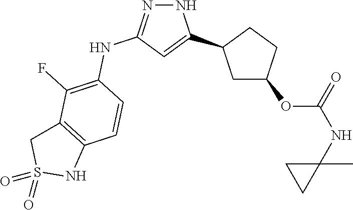 [SMILES: CC1(NC(=O)O[C@@H]2CC[C@H](c3cc(Nc4ccc5c(c4F)CS(=O)(=O)N5)n[nH]3)C2)CC1]

Enantiomer 1 (Example 388a, (1R,3S)-3-(3-((4-fluoro-2,2-dioxido-1,3-dihydrobenzo[c]isothiazol-5-yl)amino)-1H-pyrazol-5-yl)cyclopentyl(1-methylcyclopropyl)carbamate, 100% ee); Retention time: 5.133 min. ¹H NMR (500 MHz, DMSO-d₆) δ 11.67 (s, 1H), 10.20 (s, 1H), 7.94 (s, 1H), 7.85 (s, 1H), 7.34 (s, 1H), 6.58 (d, J=8.6 Hz, 1H), 5.66 (s, 1H), 4.98 (s, 1H), 4.58 (s, 2H), 3.08-2.95 (m, 1H), 2.50-2.42 (m, 1H), 2.05-1.95 (m, 1H), 1.94-1.85 (m, 1H), 1.73-1.64 (m, 2H), 1.59-1.51 (m, 1H), 1.23 (s, 3H), 0.61-0.57 (m, 2H), 0.48-0.46 (m, 2H). LC-MS (ESI): m/z [M+H]⁺=450.3.

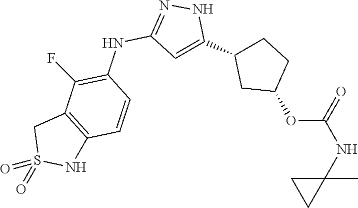 [SMILES: CC1(NC(=O)O[C@H]2CC[C@@H](c3cc(Nc4ccc5c(c4F)CS(=O)(=O)N5)n[nH]3)C2)CC1]

Enantiomer 2 (Example 388b, (1S,3R)-3-(3-((4-fluoro-2,2-dioxido-1,3-dihydrobenzo[c]isothiazol-5-yl)amino)-1H-pyrazol-5-yl)cyclopentyl(1-methylcyclopropyl)carbamate, 100% ee); Retention time: 7.045 min. ¹H NMR (500 MHz, DMSO-d₆) δ 11.67 (s, 1H), 10.20 (s, 1H), 7.94 (s, 1H), 7.85 (s, 1H), 7.34 (s, 1H), 6.58 (d, J=8.6 Hz, 1H), 5.66 (s, 1H), 4.98 (s, 1H), 4.58 (s, 2H), 3.08-2.95 (m, 1H), 2.50-2.42 (m, 1H), 2.05-1.95 (m, 1H), 1.94-1.85 (m, 1H), 1.73-1.64 (m, 2H), 1.59-1.51 (m, 1H), 1.23 (s, 3H), 0.61-0.57 (m, 2H), 0.48-0.46 (m, 2H). LC-MS (ESI): m/z [M+H]⁺=450.3.

Chiral analytical method: Column: CHIRALPAK IE 4.6 mm×250 mm, 5 μm; Mobile phase: A for MTBE (0.1% 2M NH₃ MeOH) and B for MEOH/DCM (50/50); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MEOH/DCM (50/50); Gradient: Mobile Phase A: Mobile Phase B=60: 40 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 389: cis-3-(3-((4-fluoro-1-(3-hydroxypropyl)-2,2-dioxido-1,3-dihydrobenzo[c]isothiazol-5-yl)amino)-1H-pyrazol-5-yl)cyclopentyl isopropylcarbamate

### Step 1:5-bromo-4-fluoro-1-(3-hydroxypropyl)-1,3-dihydrobenzo[c]isothiazole 2,2-dioxide

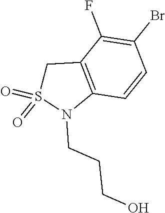 [SMILES: O=S1(=O)Cc2c(ccc(Br)c2F)N1CCCO]

To a solution of 5-bromo-4-fluoro-1,3-dihydrobenzo[c]isothiazole 2,2-dioxide (50 mg, 0.19 mmol) in ACN (10 mL) was added K₂CO₃ (52.8 mg, 0.8 mmol) and 3-bromopropan-1-ol (39 mg, 0.28 mmol). The mixture was stirred at 60° C. for 16 h. The mixture was concentrated under vacuum. The residue was purified by silica gel column chromatography, eluting with EA/PE (0-55%) to afford the product (60 mg, 98.2%). LC-MS (ESI): m/z [M+H]⁺=324.2.

### Step 2: cis-3-(3-((4-fluoro-1-(3-hydroxypropyl)-2,2-dioxido-1,3-dihydrobenzo[c]isothiazol-5-yl)amino)-1H-pyrazol-5-yl)cyclopentyl isopropylcarbamate

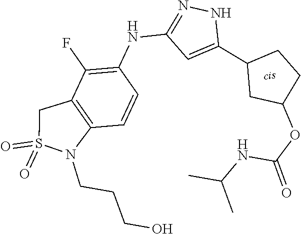 [SMILES: CC(C)NC(=O)OC1CCC(c2cc(Nc3ccc4c(c3F)CS(=O)(=O)N4CCCO)n[nH]2)C1]

A mixture of 5-bromo-4-fluoro-1-(3-hydroxypropyl)-1,3-dihydrobenzo[c]isothiazole 2,2-dioxide (60 mg, 0.18 mmol), cis-3-(3-amino-1H-pyrazol-5-yl)cyclopentyl isopropylcarbamate (37.5 mg, 0.15 mmol), Brettphos Pd G3 (16.3 mg, 0.018 mmol) and K₂CO₃ (74.5 mg, 0.54 mmol) in t-BuOH (5 mL) was stirred at 110° C. for 16 h under a nitrogen atmosphere. The mixture was concentrated under vacuum. The residue was purified by silica gel column chromatography, eluting with EA/PE (0-100%) to afford the product (65 mg, crude). The crude product was purified by Prep-HPLC to afford the product (21.68 mg, 22.8%). ¹H NMR (500 MHz, DMSO-d₆) δ 11.70 (s, 1H), 8.06-7.95 (m, 1H), 7.89 (s, 1H), 6.94 (d, J=10 Hz, 1H), 6.70 (d, J=10 Hz, 1H), 5.67 (s, 1H), 5.04-4.94 (m, 1H), 4.70 (s, 2H), 4.59 (t, J=5 Hz, 1H), 3.69-3.47 (m, 5H), 3.08-2.96 (m, 1H), 2.47-2.40 (m, 1H), 2.05-1.95 (m, 1H), 1.94-1.85 (m, 1H), 1.84-1.77 (m, 2H), 1.76-1.53 (m, 3H), 1.07-0.97 (m, 6H). LC-MS (ESI): m/z [M+H]⁺=496.2.

### Example 390: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl(3-methyloxetan-3-yl)carbamate

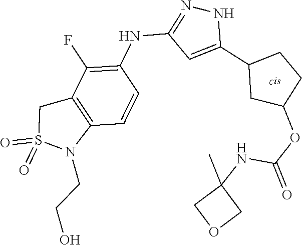 [SMILES: CC1(NC(=O)OC2CCC(c3cc(Nc4ccc5c(c4F)CS(=O)(=O)N5CCO)n[nH]3)C2)COC1]

The titled compound was synthesized in the procedures similar to Example 389. ¹H NMR (500 MHz, DMSO-d₆) δ 11.71 (s, 1H), 8.05-7.92 (m, 1H), 7.90 (s, 1H), 7.67-7.58 (m, 1H), 6.76 (d, J=10 Hz, 1H), 5.68 (s, 1H), 5.05-4.97 (m, 1H), 4.95 (t, J=5 Hz, 1H), 4.69 (s, 2H), 4.61-4.51 (m, 2H), 4.29-4.18 (m, 2H), 3.66-2.59 (m, 2H), 3.56-3.51 (m, 2H), 3.08-2.98 (m, 1H), 2.48-2.42 (m, 1H), 2.05-1.96 (m, 1H), 1.95-1.84 (m, 1H), 1.78-1.65 (m, 2H), 1.64-1.56 (m, 1H), 1.47 (s, 3H). LC-MS (ESI): m/z [M+H]⁺=510.2.

### Example 391: cis-3-(3-((1-methyl-2,2-dioxido-1,3-dihydrobenzo[c]isothiazol-5-yl)amino)-1H-pyrazol-5-yl)cyclopentyl tert-butylcarbamate

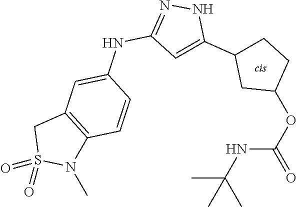 [SMILES: CN1c2ccc(Nc3cc(C4CCC(OC(=O)NC(C)(C)C)C4)[nH]n3)cc2CS1(=O)=O]

The titled compound was synthesized in the procedures similar to Example 324 in a racemic form, which was further separated by Chiral Prep-HPLC to give:

Enantiomer 1 (Example 391a, 100% ee); Retention time: 4.493 min. ¹H NMR (500 MHz, DMSO-d₆) δ 11.62 (s, 1H), 8.22 (s, 1H), 7.44-7.39 (m, 1H), 7.29-7.22 (m, 1H), 6.82-6.71 (m, 2H), 5.60-5.53 (m, 1H), 5.01-4.93 (m, 1H), 4.56 (s, 2H), 3.06-2.97 (m, 1H), 2.95 (s, 3H), 2.46-2.39 (m, 1H), 2.05-1.95 (m, 1H), 1.93-1.83 (m, 1H), 1.77-1.65 (m, 2H), 1.62-1.51 (m, 1H), 1.21 (s, 9H). LC-MS (ESI): m/z [M+H]⁺=448.2.

Enantiomer 2 (Example 391b, 100% ee); Retention time: 4.688 min. ¹H NMR (500 MHz, DMSO-d₆) δ 11.62 (s, 1H), 8.22 (s, 1H), 7.44-7.39 (m, 1H), 7.29-7.22 (m, 1H), 6.82-6.71 (m, 2H), 5.60-5.53 (m, 1H), 5.01-4.93 (m, 1H), 4.56 (s, 2H), 3.06-2.97 (m, 1H), 2.95 (s, 3H), 2.46-2.39 (m, 1H), 2.05-1.95 (m, 1H), 1.93-1.83 (m, 1H), 1.77-1.65 (m, 2H), 1.62-1.51 (m, 1H), 1.21 (s, 9H). LC-MS (ESI): m/z [M+H]⁺=448.2.

Chiral analytical method: Column: CHIRALPAK IE, 4.6*250 mm 5 μm; Mobile phase: A for MTBE (0.1% 2 M NH₃. MeOH) and B for MeOH: DCM=50:50; Gradient: Mobile Phase A: Mobile Phase B=20:80 (v/v); HPLC Equipment: HPLC-Agilent,Back pressure: 100 bar; Column temperature: 35° C.

Chiral Prep-HPLC Condition: CHIRALPAK IE, 4.6*250 mm 5 μm; Mobile phase: A for MTBE (0.2% 2 M NH₃. MeOH) and B for MeOH: DCM=50:50; Gradient: Mobile Phase A: Mobile Phase B=20:80 (v/v); Flow Rate: 18 mL/min, Wave Length: UV 254 nm and 280 nm, Prep-HPLC Equipment: Prep-HPLC-Gilson,Back pressure: 100 bar; Column temperature: 25° C.

### Example 392: cis-3-(3-((2,2-dioxido-1,3-dihydrobenzo[c]isothiazol-5-yl)amino)-1H-pyrazol-5-yl)cyclopentyl(3-methyltetrahydrofuran-3-yl)carbamate

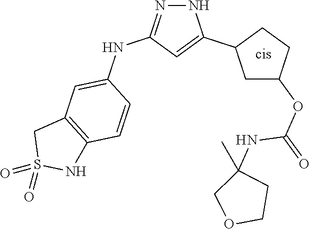 [SMILES: CC1(NC(=O)OC2CCC(c3cc(Nc4ccc5c(c4)CS(=O)(=O)N5)n[nH]3)C2)CCOC1]

The titled compound was synthesized in the procedures similar to Example 343. ¹H NMR (500 MHz, DMSO-d₆) δ 11.69 (brs, 1H), 9.77 (s, 1H), 8.23 (s, 1H), 7.36 (s, 1H), 7.23 (s, 1H), 7.14 (s, 1H), 6.71 (d, J=8.2 Hz, 1H), 5.59 (s, 1H), 5.05-4.95 (m, 1H), 4.44 (s, 2H), 3.81 (s, 1H), 3.75-3.70 (m, 2H), 3.43 (d, J=8.4 Hz, 1H), 3.08-2.99 (m, 1H), 2.47-2.41 (m, 1H), 2.23-2.10 (m, 1H), 2.05-1.97 (m, 1H), 1.94-1.85 (m, 1H), 1.78-1.66 (m, 3H), 1.64-1.53 (m, 1H), 1.32 (s, 3H). LC-MS (ESI): m/z [M+H]⁺=462.31.

### Example 393: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl(3-methyltetrahydrofuran-3-yl)carbamate

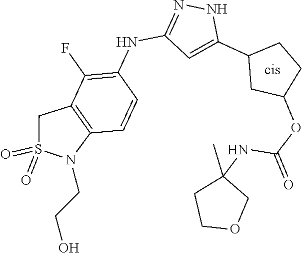 [SMILES: CC1(NC(=O)OC2CCC(c3cc(Nc4ccc5c(c4F)CS(=O)(=O)N5CCO)n[nH]3)C2)CCOC1]

The titled compound was synthesized in the procedures similar to Example 390. ¹H NMR (500 MHz, DMSO-d₆) δ 11.70 (s, 1H), 8.04-7.96 (m, 1H), 7.89 (s, 1H), 7.25-7.19 (m, 1H), 7.76 (d, J=10 Hz, 1H), 5.65 (s, 1H), 5.03-4.92 (m, 2H), 4.69 (s, 2H), 3.85-3.78 (m, 1H), 3.76-3.69 (m, 2H), 3.66-3.58 (m, 2H), 3.56-3.50 (m, 2H), 3.46-3.41 (m, 1H), 3.06-2.97 (m, 1H), 2.46-2.41 (m, 1H), 2.21-2.12 (m, 1H), 2.05-1.96 (m, 1H), 1.95-1.84 (m, 1H), 1.79-1.64 (m, 3H), 1.63-1.53 (m, 1H), 1.31 (s, 3H). LC-MS (ESI): m/z [M+H]⁺=524.2.

### Example 394: cis-3-(3-((4-fluoro-1-(2-hydroxyethyl)-2,2-dioxido-1,3-dihydrobenzo[c]isothiazol-5-yl)amino)-1H-pyrazol-5-yl)cyclopentyl(1-methoxy-2-methylpropan-2-yl)carbamate

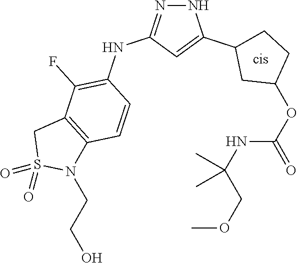 [SMILES: COCC(C)(C)NC(=O)OC1CCC(c2cc(Nc3ccc4c(c3F)CS(=O)(=O)N4CCO)n[nH]2)C1]

The titled compound was synthesized in the procedures similar to Example 391. ¹H NMR (500 MHz, DMSO-d₆) δ 11.69 (s, 1H), 8.05-7.95 (m, 1H), 7.89 (s, 1H), 6.76 (d, J=10 Hz, 1H), 6.68-6.58 (m, 1H), 5.67 (s, 1H), 5.00-4.92 (m, 2H), 4.69 (s, 2H), 3.66-3.59 (m, 2H), 3.57-3.50 (m, 2H), 3.31-3.29 (m, 2H), 3.24 (s, 3H), 3.05-2.96 (m, 1H), 2.48-2.41 (m, 1H), 2.04-1.95 (m, 1H), 1.93-1.82 (m, 1H), 1.76-1.64 (m, 2H), 1.61-1.52 (m, 1H), 1.16 (s, 6H). LC-MS (ESI): m/z [M+H]⁺=526.2.

### Example 395: cis-3-(3-((6-fluoro-1,1-dioxido-2,3-dihydrobenzo[d]isothiazol-5-yl)amino)-1H-pyrazol-5-yl)cyclopentyl tert-butylcarbamate

### Step 1: methyl 5-bromo-2-(chlorosulfonyl)-4-fluorobenzoate

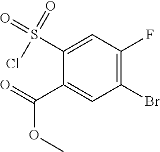 [SMILES: COC(=O)c1cc(Br)c(F)cc1S(=O)(=O)Cl]

To a suspension of methyl 2-amino-5-bromo-4-fluorobenzoate (3.2 g, 12.1 mmol) in acetic acid (20 mL) and hydrochloric acid (12 M, 54 mL), a solution of sodium nitrite (918.4 mg, 13.31 mmol) in 8 mL water was added dropwise at 0° C. The resulting mixture was kept below 5° C. and stirred for 30 mins after addition. To the reaction mixture CuCl₂ (813 mg, 6.05 mmol), sodium bisulfite (18.9 g, 181.5 mmol) and hydrochloric acid solution (6 M, 88 mL) were added sequentially. The reaction mixture was warmed to room temperature and stirred for 3 h. The resulting mixture was extracted with EA (50 mL×3). The combined organic layers were washed with brine, dried over Na₂SO₄, and filtered. The filtrated was concentrated and dried over in vacuum to afford the crude product (3.0 g), which was used in the next step without further purification.

### Step 2:5-bromo-6-fluorobenzo[d]isothiazol-3 (2H)-one 1,1-dioxide

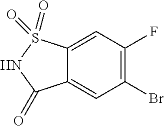 [SMILES: O=C1NS(=O)(=O)c2cc(F)c(Br)cc21]

To a mixture of methyl 5-bromo-2-(chlorosulfonyl)-4-fluorobenzoate (3 g crude) was dissolved in THF (10 mL) was added, aqueous ammonium (25%, 20 mL). The reaction mixture was stirred at room temperature for 16 h. The resulting mixture was concentrated under vacuum, and the residue was triturated with EA and filtered to afford the crude product (2.0 g). LC-MS (ESI): m/z [M−H]=277.9.

### Step 3:5-bromo-6-fluoro-2,3-dihydrobenzo[d]isothiazole 1,1-dioxide

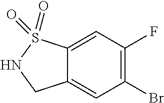 [SMILES: O=S1(=O)NCc2cc(Br)c(F)cc21]

To a stirring solution of 5-bromo-6-fluoro-2,3-dihydrobenzo[d]isothiazole 1,1-dioxide (300 mg, 1.08 mmol) in THF (10 mL) was added BH₃-Me₂S (2 M, 2.7 mL, 5.4 mmol) dropwise at 0° C. under nitrogen atmosphere. The solution was heated to reflux and stirred for 16 h. The resulting mixture was cooled to 0° C., quenched with MeOH (10 mL), and concentrated under reduced pressure. The residue was purified by silica gel column chromatography, eluting with PE/EA (1:1) to afford the product (260 mg, 91%). LC-MS (ESI): m/z [M+H]⁺=266/268.

### Step 4: cis-benzyl(1-(tert-butyl)-3-(3-(((4-nitrophenoxy) carbonyl)oxy) cyclopentyl)-1H-pyrazol-5-yl)carbamate

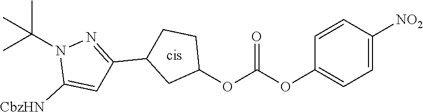 [SMILES: CC(C)(C)n1nc(C2CCC(OC(=O)Oc3ccc([N+](=O)[O-])cc3)C2)cc1NC(=O)OCc1ccccc1]

To a solution of cis-benzyl(1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 5: cis-benzyl(5-(3-(((4-nitrophenoxy) carbonyl)oxy) cyclopentyl)-1H-pyrazol-3-yl)carbamate

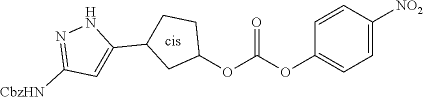 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl(1-(tert-butyl)-3-(3-(((4-nitrophenoxy) carbonyl)oxy) cyclopentyl)-1H-pyrazol-5-yl)carbamate (30 g, 57.5 mmol) was added 100 mL formic acid. The resulting mixture was stirred at 75° C. for over night. The solvent was removed under vacuum to yield 26 g of crude product, which was directly used for the next step without further purification. LC-MS (ESI): m/z [M+H]⁺=467.2.

### Step 6: cis-3-(3-(((benzyloxy) carbonyl)amino)-1H-pyrazol-5-yl)cyclopentyl tert-butylcarbamate

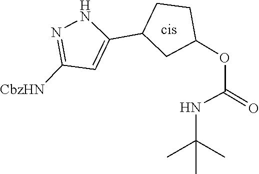 [SMILES: CC(C)(C)NC(=O)OC1CCC(c2cc(NC(=O)OCc3ccccc3)n[nH]2)C1]

To a solution of cis-benzyl(5-(3-(((4-nitrophenoxy) carbonyl)oxy) cyclopentyl)-1H-pyrazol-3-yl)carbamate (10 g, 21.5 mmol) in 100 mL THF was added tert-Butylamine (15.7 g, 215 mmol). The resulting mixture was stirred at room temperature for 12 h. The resulting mixture was concentrated under reduced pressure, and the residue was purified by silica gel chromatography (PE/EA=2:1) to afford the product (5.8 g, 67% yield). LC-MS (ESI): m/z [M+H]⁺=401.3.

### Step 7: cis-3-(3-amino-1H-pyrazol-5-yl)cyclopentyl tert-butylcarbamate

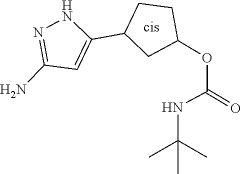 [SMILES: CC(C)(C)NC(=O)OC1CCC(c2cc(N)n[nH]2)C1]

To a solution of cis-3-(3-(((benzyloxy) carbonyl)amino)-1H-pyrazol-5-yl)cyclopentyl tert-butylcarbamate (5.8 g, 14 mmol) in THF (100 mL) was added Pd/C (10%, wet, 3 g). The suspension was degassed and purged with hydrogen 3 times. The resulting mixture was stirred at room temperature under a hydrogen balloon for 5 h. The mixture was filtered, and the filter cake was washed with EA (200 mL×3). The filtrate was concentrated under reduced pressure to afford the product (3.5 g, 95% yield). LC-MS (ESI): m/z [M+H]⁺=267.3.

### Step 8: cis-3-(3-((6-fluoro-1,1-dioxido-2,3-dihydrobenzo[d]isothiazol-5-yl)amino)-1H-pyrazol-5-yl)cyclopentyl tert-butylcarbamate

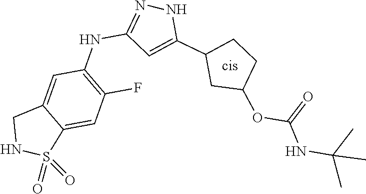 [SMILES: CC(C)(C)NC(=O)OC1CCC(c2cc(Nc3cc4c(cc3F)S(=O)(=O)NC4)n[nH]2)C1]

A mixture of 5-bromo-6-fluoro-2,3-dihydrobenzo[d]isothiazole 1,1-dioxide (140 mg, 0.53 mmol), cis-3-(3-amino-1H-pyrazol-5-yl)cyclopentyl tert-butylcarbamate (114.8 mg, 0.43 mmol), Brettphos Pd G3 (48.4 mg, 0.053 mmol), K₂CO₃ (219 mg, 1.59 mmol) in t-BuOH (10 mL) was stirred at 110° C. for 16 h under N₂ atmosphere. The mixture was concentrated under reduced pressure. The residue was purified by silica gel column chromatography, eluting with PE/EA (2:1~1:9) to afford the crude product. The residue 50 mg was purified by Prep-HPLC (Waters XSelect C18: RD-CO-094 column, eluting with a gradient of acetonitrile/water containing 0.1% FA, 20%-40%) to afford the product in a racemic form, which was further separated by Chiral Prep-HPLC to give:

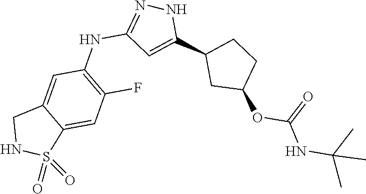 [SMILES: CC(C)(C)NC(=O)O[C@@H]1CC[C@H](c2cc(Nc3cc4c(cc3F)S(=O)(=O)NC4)n[nH]2)C1]

Enantiomer 1 (Example 395a, (1R,3S)-3-(3-((6-fluoro-1,1-dioxido-2,3-dihydrobenzo[d]isothiazol-5-yl)amino)-1H-pyrazol-5-yl)cyclopentyl tert-butylcarbamate, 100% ee); Retention time: 9.985 min. ¹H NMR (500 MHz, DMSO-d₆) δ 11.98 (s, 1H), 8.74 (d, J=2.5 Hz, 1H), 8.15 (d, J=7.4 Hz, 1H), 7.65-7.63 (m, 2H), 6.76 (s, 1H), 5.84 (d, J=2.1 Hz, 1H), 5.02-4.94 (m, 1H), 4.30 (d, J=4.8 Hz, 2H), 3.11-3.00 (m, 1H), 2.46-2.38 (m, 1H), 2.08-1.97 (m, 1H), 1.95-1.85 (m, 1H), 1.78-1.64 (m, 2H), 1.62-1.52 (m, 1H), 1.21 (s, 9H). LC-MS (ESI): m/z [M+H]⁺=452.2.

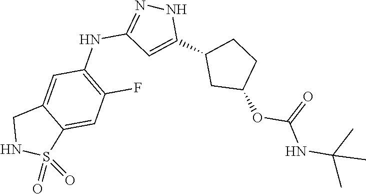 [SMILES: CC(C)(C)NC(=O)O[C@H]1CC[C@@H](c2cc(Nc3cc4c(cc3F)S(=O)(=O)NC4)n[nH]2)C1]

Enantiomer 2 (Example 395b, (1S,3R)-3-(3-((6-fluoro-1,1-dioxido-2,3-dihydrobenzo[d]isothiazol-5-yl)amino)-1H-pyrazol-5-yl)cyclopentyl tert-butylcarbamate, 100% ee); Retention time: 12.117 min. ¹H NMR (500 MHz, DMSO-d₆) δ 11.98 (s, 1H), 8.74 (d, J=2.5 Hz, 1H), 8.15 (d, J=7.4 Hz, 1H), 7.65-7.63 (m, 2H), 6.76 (s, 1H), 5.84 (d, J=2.1 Hz, 1H), 5.02-4.94 (m, 1H), 4.30 (d, J=4.8 Hz, 2H), 3.11-3.00 (m, 1H), 2.46-2.38 (m, 1H), 2.08-1.97 (m, 1H), 1.95-1.85 (m, 1H), 1.78-1.64 (m, 2H), 1.62-1.52 (m, 1H), 1.21 (s, 9H). LC-MS (ESI): m/z [M+H]⁺=452.2.

Chiral analytical method: Column: CHIRALPAK IE, 4.6*250 mm 5 μm; Mobile phase: A for Hex and B for EtOH (0.1% 2 M NH₃. MeOH); Gradient: Mobile Phase A: Mobile Phase B=60:40 (v/v); HPLC Equipment: HPLC-Agilent, Back pressure: 100 bar; Column temperature: 35° C.

Chiral Prep-HPLC Condition: CHIRALPAK IE, 20*250 mm 5 μm; Mobile phase: A for Hex and B for EtOH (0.2% 2 M NH₃. MeOH); Gradient: Mobile Phase A: Mobile Phase B=60:40 (v/v); Flow Rate: 18 mL/min, Wave Length: UV 254 nm and 280 nm, Prep-HPLC Equipment: Prep-HPLC-Gilson, Back pressure: 100 bar; Column temperature: 25° C.

### Example 396: cis-3-(3-((6-methyl-1,1-dioxido-2,3-dihydroisothiazolo[5,4-b]pyridin-5-yl)amino)-1H-pyrazol-5-yl)cyclopentyl isopropylcarbamate

### Step 1: methyl 2-(benzylthio)-5-bromo-6-methylnicotinate

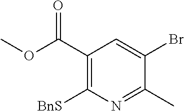

To a solution of methyl 5-bromo-2-chloro-6-methylnicotinate (2 g, 7.6 mmol) in DMF (110 mL) was added cesium carbonate (4.9 g, 15.2 mmol) and benzyl mercaptan (1.2 g, 9.9 mmol). The resulting solution was stirred for 2 h at rt. The resulting solution was extracted with 3×50 mL of EtOAc. The resulting mixture was concentrated under vacuum. The residue was purified by combi-flash PE to give the product (1.8 g, 66.7%). LCMS (ESI) m/z [M+H]⁺=352.

### Step 2: methyl 5-bromo-2-(chlorosulfonyl)-6-methylnicotinate

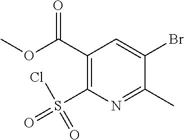

To a solution of NCS (2.7 g, 20 mmol) in MeCN (20 mL) and HCl (6 mL, 2M) was added the solution of methyl 2-(benzylthio)-5-bromo-6-methylnicotinate (1.8 g, 5 mmol) in MeCN (10 mL) at 0° C. The resulting solution was stirred for 30 min at 0° C. The resulting solution was extracted with 3×50 mL of EtOAc. The resulting mixture was concentrated under vacuum. The residue was the crude product (1.6 g, 100%).

### Step 3: methyl 5-bromo-2-(N-(2,4-dimethoxybenzyl) sulfamoyl)-6-methylnicotinate

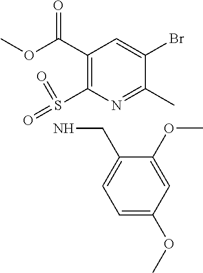

To a solution of methyl 5-bromo-2-(chlorosulfonyl)-6-methylnicotinate (1.6 g, 4.9 mmol) in DCM (25 mL) was added (2,4-dimethoxyphenyl) methanamine (1.7 g, 9.8 mmol) at 0° C. The resulting solution was stirred for 30 min at rt. The resulting mixture was concentrated under vacuum. The residue was purified by combi-flash (EtOAc/PE=20%) to give the product (1.4 g, 63.6%). LCMS (ESI) m/z [M+H]⁺=459.

### Step 4:5-bromo-N-(2,4-dimethoxybenzyl)-3-(hydroxymethyl)-6-methylpyridine-2-sulfonamide

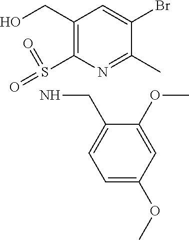

To a solution of methyl 5-bromo-2-(N-(2,4-dimethoxybenzyl) sulfamoyl)-6-methylnicotinate (1.3 g, 2.8 mmol) in THF (20 mL) was added LiBH₄ (62 mg, 5.6 mmol) at 0° C. The resulting solution was stirred for 2 h at rt. The reaction was then quenched by the addition of MeOH. The resulting mixture was concentrated under vacuum. The residue was purified by combi-flash (EtOAc/PE=20%-40%) to give the product (935 mg, 77.9%). LCMS (ESI) m/z [M+H]⁺=431.

### Step 5:5-bromo-2-(2,4-dimethoxybenzyl)-6-methyl-2,3-dihydroisothiazolo[5,4-b]pyridine 1,1-dioxide

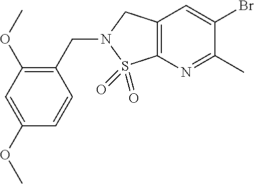 [SMILES: COc1ccc(CN2Cc3cc(Br)c(C)nc3S2(=O)=O)c(OC)c1]

To a solution of 5-bromo-N-(2,4-dimethoxybenzyl)-3-(hydroxymethyl)-6-methylpyridine-2-sulfonamide (886 mg, 2.1 mmol) in THF (15 mL) were added PPh₃ (1.1 g, 4.2 mmol). Then the solution of DIAD (832 mg, 4.2 mmol) in THF (2 mL) was added to the reaction at 0° C. The resulting solution was stirred for 1 h at rt under nitrogen atmosphere. The resulting solution was extracted with 3×30 mL of EtOAc. The resulting mixture was concentrated under vacuum. The residue was purified by combi-flash (EtOAc/PE=27%) to give the product (650 mg, 75%). LCMS (ESI) m/z [M+H]⁺=413.

### Step 6:5-bromo-6-methyl-2,3-dihydroisothiazolo[5,4-b]pyridine 1,1-dioxide

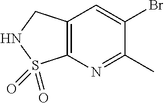

To a solution of 5-bromo-2-(2,4-dimethoxybenzyl)-6-methyl-2,3-dihydroisothiazolo[5,4-b] pyridine 1,1-dioxide (650 mg, 1.6 mmol) in DCM (9 mL) was added TFA (3 mL). The resulting solution was stirred for 1 h at rt. The resulting mixture was concentrated under vacuum. The residue was purified by combi-flash (EtOAc/PE=40%-100%) to give the product (4 g, 38.1%). ¹H NMR (400 MHz, DMSO-d₆) δ 8.36 (s, 1H), 8.10 (t, J=4.6 Hz, 1H), 4.41-4.36 (m, 2H), 2.69 (s, 3H). LCMS (ESI) m/z [M+H]⁺=262.9.

### Step 7: cis-3-(3-((6-methyl-1,1-dioxido-2,3-dihydroisothiazolo[5,4-b]pyridin-5-yl)amino)-1H-pyrazol-5-yl)cyclopentyl isopropylcarbamate

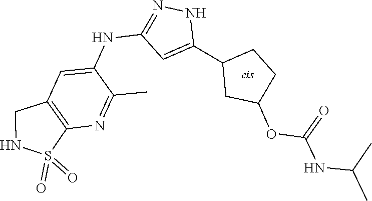 [SMILES: Cc1nc2c(cc1Nc1cc(C3CCC(OC(=O)NC(C)C)C3)[nH]n1)CNS2(=O)=O]

The titled compound was synthesized in the procedures similar to Example 34. ¹H NMR (500 MHz, DMSO-d₆) δ 12.04 (s, 1H), 8.27 (s, 1H), 8.13 (s, 1H), 7.61 (t, J=4.6 Hz, 1H), 6.94 (d, J=7.3 Hz, 1H), 5.91 (s, 1H), 5.00 (s, 1H), 4.29 (d, J=4.3 Hz, 2H), 3.65-3.53 (m, 1H), 3.15-3.06 (m, 1H), 2.54 (s, 3H), 2.48-2.43 (m, 1H), 2.11-1.86 (m, 2H), 1.79-1.55 (m, 3H), 1.13-0.95 (m, 6H). LC-MS (ESI): m/z [M+H]⁺=435.3.

### Example 397: (1s,3s)-3-(3-((6-fluoro-1,1-dioxido-2,3-dihydrobenzo[d]isothiazol-5-yl)amino)-1H-pyrazol-5-yl)cyclobutyl isopropylcarbamate

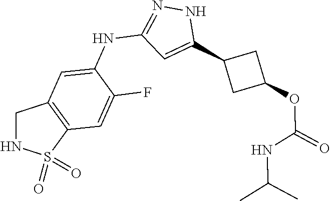 [SMILES: CC(C)NC(=O)O[C@H]1C[C@@H](c2cc(Nc3cc4c(cc3F)S(=O)(=O)NC4)n[nH]2)C1]

The titled compound was synthesized in the procedures similar to Example 50. ¹H NMR (500 MHz, DMSO-d₆) 12.06 (s, 1H), 8.76 (s, 1H), 8.13 (d, J=7.3 Hz, 1H), 7.69-7.60 (m, 2H), 7.06 (d, J=7.5 Hz, 1H), 5.88 (s, 1H), 4.86-4.78 (m, 1H), 4.30 (d, J=4.8 Hz, 2H), 3.65-3.53 (m, 1H), 3.12-3.05 (m, 1H), 2.71-2.64 (m, 2H), 2.12-2.03 (m, 2H), 1.04 (d, J=6.6 Hz, 6H). LC-MS (ESI): m/z [M+H]⁺=424.3.

### Example 398: cis-3-(3-((2,2-dioxido-1,3-dihydrobenzo[c]isothiazol-5-yl)amino)-1H-pyrazol-5-yl)cyclopentyl(4-methyltetrahydro-2H-pyran-4-yl)carbamate

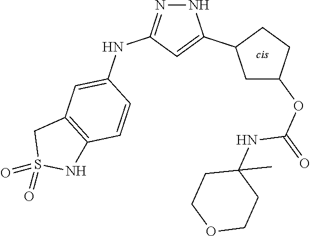 [SMILES: CC1(NC(=O)OC2CCC(c3cc(Nc4ccc5c(c4)CS(=O)(=O)N5)n[nH]3)C2)CCOCC1]

The titled compound was synthesized in the procedures similar to example 343. ¹H NMR (500 MHz, DMSO-d₆) δ 11.69 (s, 1H), 9.76 (s, 1H), 8.21 (s, 1H), 7.36 (s, 1H), 7.15 (d, J=8.3 Hz, 1H), 6.85 (s, 1H), 6.71 (d, J=8.6 Hz, 1H), 5.59 (s, 1H), 5.05-4.90 (m, 1H), 4.44 (s, 2H), 3.58-3.50 (m, 4H), 3.07-2.94 (m, 1H), 2.48-2.43 (m, 1H), 2.05-1.98 (m, 1H), 1.97-1.86 (m, 3H), 1.77-1.67 (m, 2H), 1.65-1.55 (m, 1H), 1.48-1.39 (m, 2H), 1.23 (s, 3H). LC-MS (ESI): m/z [M+H]⁺=476.31.

### Example 399: cis-3-(3-((4-fluoro-2,2-dioxido-1,3-dihydrobenzo[c]isothiazol-5-yl)amino)-1H-pyrazol-5-yl)cyclopentyl cyclobutylcarbamate

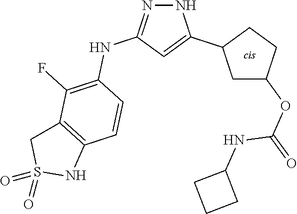 [SMILES: O=C(NC1CCC1)OC1CCC(c2cc(Nc3ccc4c(c3F)CS(=O)(=O)N4)n[nH]2)C1]

The titled compound was synthesized in the procedures similar to Example 251 in a racemic form, which was further separated by Chiral Prep-HPLC to give:

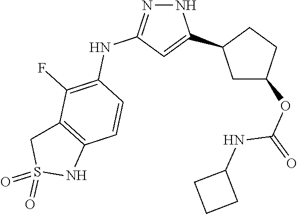 [SMILES: O=C(NC1CCC1)O[C@@H]1CC[C@H](c2cc(Nc3ccc4c(c3F)CS(=O)(=O)N4)n[nH]2)C1]

Enantiomer 1 (Example 399a, (1R,3S)-3-(3-((4-fluoro-2,2-dioxido-1,3-dihydrobenzo[c]isothiazol-5-yl)amino)-1H-pyrazol-5-yl)cyclopentyl cyclobutylcarbamate, 100% ee); Retention time: 4.38 min. ¹H NMR (500 MHz, DMSO-d₆) δ 11.67 (s, 1H), 10.24 (s, 1H), 7.92 (s, 1H), 7.83 (s, 1H), 7.36 (d, J=8.0 Hz, 1H), 6.57 (d, J=8.6 Hz, 1H), 5.66 (s, 1H), 4.97 (s, 1H), 4.56 (s, 2H), 3.98-3.90 (m, 1H), 3.11-2.96 (m, 1H), 2.45-2.38 (m, 1H), 2.15-2.05 (m, 2H), 2.03-1.95 (m, 1H), 1.94-1.79 (m, 3H), 1.75-1.65 (m, 2H), 1.62-1.46 (m, 3H). LC-MS (ESI): m/z [M+H]⁺=450.2.

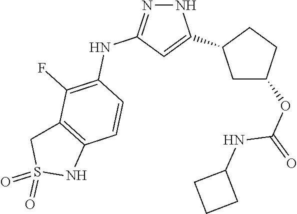 [SMILES: O=C(NC1CCC1)O[C@H]1CC[C@@H](c2cc(Nc3ccc4c(c3F)CS(=O)(=O)N4)n[nH]2)C1]

Enantiomer 2 (Example 399b, (1S,3R)-3-(3-((4-fluoro-2,2-dioxido-1,3-dihydrobenzo[c]isothiazol-5-yl)amino)-1H-pyrazol-5-yl)cyclopentyl cyclobutylcarbamate, 100% ee); Retention time: 5.48 min. ¹H NMR (500 MHz, DMSO-d₆) δ 11.67 (s, 1H), 10.24 (s, 1H), 7.92 (s, 1H), 7.83 (s, 1H), 7.36 (d, J=8.0 Hz, 1H), 6.57 (d, J=8.6 Hz, 1H), 5.66 (s, 1H), 4.97 (s, 1H), 4.56 (s, 2H), 3.98-3.90 (m, 1H), 3.11-2.96 (m, 1H), 2.45-2.38 (m, 1H), 2.15-2.05 (m, 2H), 2.03-1.95 (m, 1H), 1.94-1.79 (m, 3H), 1.75-1.65 (m, 2H), 1.62-1.46 (m, 3H). LC-MS (ESI): m/z [M+H]⁺=450.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=20: 80 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 400: cis-3-(3-((4-fluoro-2,2-dioxido-1,3-dihydrobenzo[c]isothiazol-5-yl)amino)-1H-pyrazol-5-yl)cyclopentyl cyclopentylcarbamate

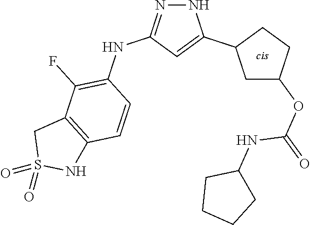 [SMILES: O=C(NC1CCCC1)OC1CCC(c2cc(Nc3ccc4c(c3F)CS(=O)(=O)N4)n[nH]2)C1]

The titled compound was synthesized in the procedures similar to Example 251 in a racemic form, which was further separated by Chiral Prep-HPLC to give:

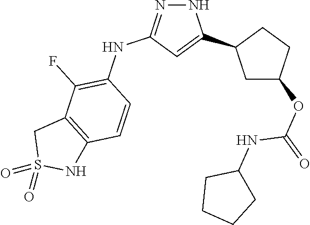 [SMILES: O=C(NC1CCCC1)O[C@@H]1CC[C@H](c2cc(Nc3ccc4c(c3F)CS(=O)(=O)N4)n[nH]2)C1]

Enantiomer 1 (Example 400a, (1R,3S)-3-(3-((4-fluoro-2,2-dioxido-1,3-dihydrobenzo[c]isothiazol-5-yl)amino)-1H-pyrazol-5-yl)cyclopentyl cyclopentylcarbamate, 100% ee); Retention time: 4.42 min. ¹H NMR (500 MHz, DMSO-d₆) δ 11.67 (s, 1H), 10.20 (s, 1H), 7.93 (s, 1H), 7.83 (s, 1H), 7.05 (d, J=6.7 Hz, 1H), 5.66 (s, 1H), 4.99 (s, 1H), 4.57 (s, 2H), 3.80-3.70 (m, 1H), 3.08-2.96 (m, 1H), 2.46-2.38 (m, 1H), 2.05-1.95 (m, 1H), 1.95-1.85 (m, 1H), 1.80-1.65 (m, 4H), 1.59-1.50 (m, 3H), 1.50-1.40 (m, 2H), 1.40-1.30 (m, 2H). LC-MS (ESI): m/z [M+H]⁺=464.2.

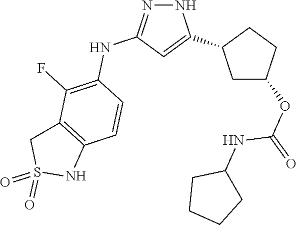 [SMILES: O=C(NC1CCCC1)O[C@H]1CC[C@@H](c2cc(Nc3ccc4c(c3F)CS(=O)(=O)N4)n[nH]2)C1]

Enantiomer 2 (Example 400b, (1S,3R)-3-(3-((4-fluoro-2,2-dioxido-1,3-dihydrobenzo[c]isothiazol-5-yl)amino)-1H-pyrazol-5-yl)cyclopentyl cyclopentylcarbamate, 100% ee); Retention time: 5.78 min. ¹H NMR (500 MHz, DMSO-d₆) δ 11.67 (s, 1H), 10.20 (s, 1H), 7.93 (s, 1H), 7.83 (s, 1H), 7.05 (d, J=6.7 Hz, 1H), 5.66 (s, 1H), 4.99 (s, 1H), 4.57 (s, 2H), 3.80-3.70 (m, 1H), 3.08-2.96 (m, 1H), 2.46-2.38 (m, 1H), 2.05-1.95 (m, 1H), 1.95-1.85 (m, 1H), 1.80-1.65 (m, 4H), 1.59-1.50 (m, 3H), 1.50-1.40 (m, 2H), 1.40-1.30 (m, 2H). LC-MS (ESI): m/z [M+H]⁺=464.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=20: 80 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=20: 80 (v/v); Flow Rate: 18 mL/min, Wave Length: UV 270 nm and 300 nm, Prep-HPLC Equipment: Prep-HPLC-Gilson, Back pressure: 100 bar; Column temperature: RT.

### Example 401: cis-3-(3-((1-cyclopropyl-4-fluoro-2,2-dioxido-1,3-dihydrobenzo[c]isothiazol-5-yl)amino)-1H-pyrazol-5-yl)cyclopentyl(1-methoxy-2-methylpropan-2-yl)carbamate

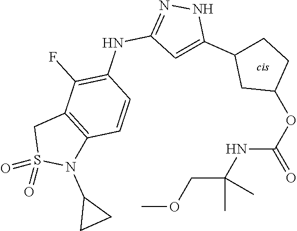 [SMILES: COCC(C)(C)NC(=O)OC1CCC(c2cc(Nc3ccc4c(c3F)CS(=O)(=O)N4C3CC3)n[nH]2)C1]

The titled compound was synthesized in the procedures similar to Example 362. ¹H NMR (500 MHz, DMSO-d₆) δ 11.74 (s, 1H), 8.02 (s, 1H), 7.93 (s, 1H), 6.83 (d, J=8.7 Hz, 1H), 6.63 (s, 1H), 5.68 (s, 1H), 4.96 (s, 1H), 4.72 (d, J=22.4 Hz, 2H), 3.31 (d, J=4.8 Hz, 4H), 3.07-2.94 (m, 1H), 2.45-2.38 (m, 1H), 2.06-1.95 (m, 1H), 1.94-1.83 (m, 1H), 1.78-1.64 (m, 2H), 1.62-1.53 (m, 1H), 1.16 (s, 6H), 0.98-0.90 (m, 2H), 0.90-0.79 (m, 2H). LC-MS (ESI): m/z [M+H]⁺=522.2

### Example 402: cis-3-(3-((4-fluoro-2,2-dioxido-1,3-dihydrobenzo[c]isothiazol-5-yl)amino)-1H-pyrazol-5-yl)cyclopentyl(4-methyltetrahydro-2H-pyran-4-yl)carbamate

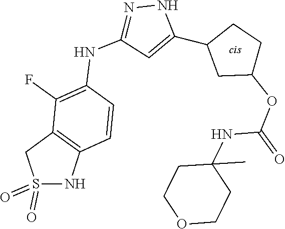 [SMILES: CC1(NC(=O)OC2CCC(c3cc(Nc4ccc5c(c4F)CS(=O)(=O)N5)n[nH]3)C2)CCOCC1]

The titled compound was synthesized in the procedures similar to Example 251. ¹H NMR (500 MHz, DMSO-d₆) δ 11.65 (s, 1H), 7.87 (s, 1H), 7.78 (s, 1H), 6.84 (s, 1H), 6.55 (d, J=8.5 Hz, 1H), 5.65 (s, 1H), 4.97 (s, 1H), 4.52 (s, 2H), 3.58-3.44 (m, 4H), 3.09-2.90 (m, 1H), 2.45-2.38 (m, 1H), 2.04-1.83 (m, 4H), 1.80-1.65 (m, 2H), 1.59 (s, 1H), 1.47-1.39 (m, 2H), 1.23 (s, 3H). LC-MS (ESI): m/z [M+H]⁺=494.2.

### Example 403: cis-3-(3-((2,2-dioxido-1,3-dihydrobenzo[c]isothiazol-5-yl)amino)-1H-pyrazol-5-yl)cyclopentyl tert-butylcarbamate

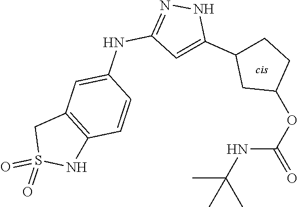 [SMILES: CC(C)(C)NC(=O)OC1CCC(c2cc(Nc3ccc4c(c3)CS(=O)(=O)N4)n[nH]2)C1]

The titled compound was synthesized in the procedures similar to Example 343 in a racemic form, which was further separated by Chiral Prep-HPLC to give:

Enantiomer 1 (Example 403a, 100% ee); Retention time: 4.508 min. ¹H NMR (500 MHz, DMSO-d₆) δ 11.61 (s, 1H), 9.74 (s, 1H), 8.19 (s, 1H), 7.38 (s, 1H), 7.16 (d, J=10 Hz, 1H), 6.81-6.68 (m, 2H), 5.57 (s, 1H), 5.01-4.91 (m, 1H), 4.43 (s, 2H), 3.06-2.97 (m, 1H), 2.46-2.40 (m, 1H), 2.05-1.95 (m, 1H), 1.94-1.83 (m, 1H), 1.76-1.64 (m, 2H), 1.62-1.51 (m, 1H), 1.21 (s, 9H). LC-MS (ESI): m/z [M+H]⁺=434.2.

Enantiomer 2 (Example 403b, 100% ee); Retention time: 5.975 min. ¹H NMR (500 MHz, DMSO-d₆) δ 11.61 (s, 1H), 9.74 (s, 1H), 8.19 (s, 1H), 7.38 (s, 1H), 7.16 (d, J=10 Hz, 1H), 6.81-6.67 (m, 2H), 5.57 (s, 1H), 5.01-4.90 (m, 1H), 4.43 (s, 2H), 3.06-2.97 (m, 1H), 2.46-2.40 (m, 1H), 2.05-1.96 (m, 1H), 1.94-1.84 (m, 1H), 1.76-1.63 (m, 2H), 1.62-1.51 (m, 1H), 1.21 (s, 9H). LC-MS (ESI): m/z [M+H]⁺=434.2.

Chiral analytical method: Column: CHIRALPAK IE, 4.6*250 mm 5 μm; Mobile phase: A for Hex and B for EtOH (0.1% 2 M NH₃. MeOH); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: CHIRALPAK IE, 4.6*250 mm 5 μm; Mobile phase: A for Hex and B for EtOH (0.2% 2 M NH₃. MeOH); Gradient: Mobile Phase A: Mobile Phase B=20:80 (v/v); Flow Rate: 18 mL/min, Wave Length: UV 254 nm and 280 nm, Prep-HPLC Equipment: Prep-HPLC-Gilson, Back pressure: 100 bar; Column temperature: 25° C.

### Example 404: cis-3-(3-((6-fluoro-2,2-dioxido-1,3-dihydrobenzo[c]isothiazol-5-yl)amino)-1H-pyrazol-5-yl)cyclopentyl tert-butylcarbamate

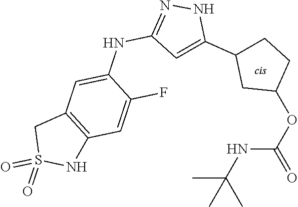 [SMILES: CC(C)(C)NC(=O)OC1CCC(c2cc(Nc3cc4c(cc3F)NS(=O)(=O)C4)n[nH]2)C1]

The titled compound was synthesized in the procedures similar to Example 343 in a racemic form, which was further separated by Chiral Prep-HPLC to give:

Enantiomer 1 (Example 404a, 100% ee); Retention time: 5.098 min. ¹H NMR (500 MHz, DMSO-d₆) δ 11.65 (s, 1H), 10.08 (s, 1H), 8.10-7.98 (m, 1H), 7.90-7.83 (m, 1H), 6.82-6.66 (m, 2H), 5.68 (s, 1H), 5.01-4.92 (m, 1H), 4.45 (s, 2H), 3.06-2.95 (m, 1H), 2.46-2.41 (m, 1H), 2.04-1.95 (m, 1H), 1.93-1.83 (m, 1H), 1.77-1.63 (m, 2H), 1.61-1.51 (m, 1H), 1.21 (s, 9H). LC-MS (ESI): m/z [M+H]⁺=452.2.

Enantiomer 2 (Example 404b, 100% ee); Retention time: 7.241 min. ¹H NMR (500 MHz, DMSO-d₆) δ 11.65 (s, 1H), 10.16 (s, 1H), 8.10-7.98 (m, 1H), 7.90-7.83 (m, 1H), 6.82-6.66 (m, 2H), 5.68 (s, 1H), 5.01-4.92 (m, 1H), 4.45 (s, 2H), 3.06-2.95 (m, 1H), 2.46-2.41 (m, 1H), 2.04-1.95 (m, 1H), 1.93-1.83 (m, 1H), 1.77-1.63 (m, 2H), 1.61-1.52 (m, 1H), 1.21 (s, 9H). LC-MS (ESI): m/z [M+H]⁺=452.2.

Chiral analytical method: Column: Amylose 4.6*250 mm; Mobile phase: A for Hex and B for (EtOH) (0.1% FA); Gradient: Mobile Phase A: Mobile Phase B=20:80 (v/v); HPLC Equipment: HPLC-Agilent, Back pressure: 100 bar; Column temperature: 35° C.

Chiral Prep-HPLC Condition: Amylose 4.6*250 mm; Mobile phase: A for Hex and B for (EtOH) (0.1% FA); Gradient: Mobile Phase A: Mobile Phase B=20:80 (v/v); Flow Rate: 18 mL/min, Wave Length: UV 254 nm and 280 nm, Prep-HPLC Equipment: Prep-HPLC-Gilson, Back pressure: 100 bar; Column temperature: 25° C.

### Example 405: cis-3-(3-((4-hydroxy-1,1-dioxido-2,3-dihydrobenzo[d]isothiazol-5-yl)amino)-1H-pyrazol-5-yl)cyclopentyl isopropylcarbamate

### Step 1:4-(benzyloxy)-5-bromo-2-(4-methoxybenzyl)-2,3-dihydrobenzo[d]isothiazole 1,1-dioxide

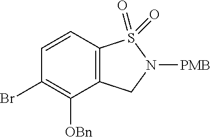

To a solution of phenylmethanol (170 mg, 1.56 mmol) in THF (3 mL) was added NaH (60% dispersion in mineral oil, 80 mg, 1.95 mmol), stirred for 1 hour at room temperature, this solution was added to a solution of 5-bromo-4-fluoro-2-(4-methoxybenzyl)-2,3-dihydrobenzo[d]isothiazole 1,1-dioxide (300 mg, 0.78 mmol) in THF (7 mL), stirred another 3 hours at 40° C. The resulting mixture was cooled to room temperature, quenched with saturated NH₄Cl aqueous, extracted with EtOAc, the combined organic phase was washed with brine, dried over Na₂SO₄, filtered, the filtrate was concentrated under reduce pressure, purified by silica gel column chromatography, eluting with PE/EA (3:1) to afford the product (9.33 g, 85%). LC-MS (ESI): m/z [M+Na]⁺=496.2.

### Step 2: cis-3-(3-((4-(benzyloxy)-2-(4-methoxybenzyl)-1,1-dioxido-2,3-dihydrobenzo[d]isothiazol-5-yl)amino)-1H-pyrazol-5-yl)cyclopentyl isopropylcarbamate

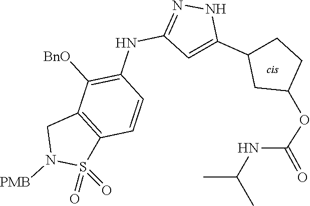

To a solution of 4-(benzyloxy)-5-bromo-2-(4-methoxybenzyl)-2,3-dihydrobenzo[d]isothiazole 1,1-dioxide (125 mg, 0.264 mmol) and cis-3-(3-amino-1H-pyrazol-5-yl)cyclopentyl isopropylcarbamate (67 mg, 0.264 mmol) in t-BuOH (3 mmol) was added BrettPhos Pd G3 (24 mg, 0.026 mmol) and K₂CO₃ (110 mg, 0.792 mmol), the reaction mixture was stirred for 3 hours at 105° C. under nitrogen. The mixture was cooled to room temperature, filtered, and the filtrate was concentrated under reduced pressure. The residue was purified by silica gel column chromatography, eluting with PE/EA (1:4) to afford the product (74 mg, 43%). LC-MS (ESI): m/z [M+H]⁺=646.3.

### Step 3: cis-3-(3-((4-hydroxy-2-(4-methoxybenzyl)-1,1-dioxido-2,3-dihydrobenzo[d]isothiazol-5-yl)amino-1H-pyrazol-5-yl)cyclopentyl isopropylcarbamate

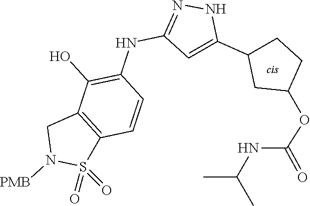

To a solution of cis-3-(3-((4-(benzyloxy)-2-(4-methoxybenzyl)-1,1-dioxido-2,3-dihydrobenzo[d]isothiazol-5-yl)amino)-1H-pyrazol-5-yl)cyclopentyl isopropylcarbamate (74 mg, 0.115 mmol) in THF/H₂O (10 mL/1 mL) was added Pd/C (10% on carbon, 10 mg), the reaction mixture was stirred for 1 hour under 1 atm hydrogen at room temperature. The resulting mixture was filtered, the filtrate was concentrated under reduce pressure, dried over in vacuum, to afford the crude product without further purification (59 mg crude). LC-MS (ESI): m/z [M+H]⁺=556.2.

### Step 4: cis-3-(3-((4-hydroxy-1,1-dioxido-2,3-dihydrobenzo[d]isothiazol-5-yl)amino)-1H-pyrazol-5-yl)cyclopentyl isopropylcarbamate

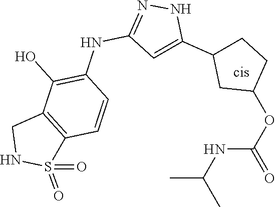 [SMILES: CC(C)NC(=O)OC1CCC(c2cc(Nc3ccc4c(c3O)CNS4(=O)=O)n[nH]2)C1]

To a solution of cis-3-(3-((4-hydroxy-2-(4-methoxybenzyl)-1,1-dioxido-2,3-dihydrobenzo[d]isothiazol-5-yl)amino)-1H-pyrazol-5-yl)cyclopentyl isopropylcarbamate (59 mg, 0.106 mmol) in DCM (5 mL) was added TFA (1.5 mL), the reaction mixture was stirred for 4 hours at room temperature. The resulting mixture was concentrated under reduce pressure, the residue was purified by prep-HPLC (Waters X-Select Prep C18 OBD column, eluting with 27%-42% of water (containing 0.1% FA) in acetonitrile) to afford the product (8.0 mg, 22%). ¹H NMR (500 MHz, DMSO-d₆) δ=11.87 (s, 1H), 10.00 (s, 1H), 7.99 (d, J=8.4, 1H), 7.91 (s, 1H), 7.44 (t, J=5.1, 1H), 7.14 (d, J=8.4, 1H), 6.95 (d, J=7.2, 1H), 5.84 (s, 1H), 5.00 (m, 1H), 4.26 (d, J=5.2, 2H), 3.58 (m, 1H), 3.18-2.97 (m, 1H), 2.45 (m, 1H), 2.07-1.97 (m, 1H), 1.97-1.84 (m, 1H), 1.72 (m, 2H), 1.60 (m, 1H), 1.03 (m, 6H). LC-MS (ESI): m/z [M+H]⁺=436.34.

### Example 406: cis-3-(3-((6-fluoro-4-hydroxy-1,1-dioxido-2,3-dihydrobenzo[d]isothiazol-5-yl)amino)-1H-pyrazol-5-yl)cyclopentyl isopropylcarbamate

### Step 1:4-(benzyloxy)-5-bromo-6-fluoro-2,3-dihydrobenzo[d]isothiazole 1,1-dioxide

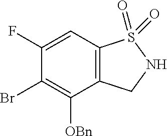 [SMILES: O=S1(=O)NCc2c1cc(F)c(Br)c2OCc1ccccc1]

To a solution of phenylmethanol (244 mg, 2.26 mmol) in THF (10 mL) was added NaH (60% dispersion in mineral oil, 115 mg, 2.83 mmol), stirred for 1 hour at room temperature, this solution was added to a solution of 5-bromo-4,6-difluoro-2,3-dihydrobenzo[d]isothiazole 1,1-dioxide (320 mg, 1.13 mmol) in THF (5 mL), stirred another 3 hours at 40° C. The resulting mixture was cooled to room temperature, quenched with saturated NH₄Cl aqueous, extracted with EtOAc, the combined organic phase was washed with brine, dried over Na₂SO₄, filtered, the filtrate was concentrated under reduce pressure, purified by silica gel column chromatography, eluting with PE/EA (1:1) to afford the product (150 mg, 36%). ¹H NMR (500 MHz, DMSO-d₆) δ=8.14 (t, J=4.9, 1H), 7.84 (d, J=6.4, 1H), 7.55-7.51 (m, 2H), 7.46-7.39 (m, 3H), 5.21 (s, 2H), 4.43-4.38 (m, 2H). LC-MS (ESI): m/z [M+H]⁺=372.1.

### Step 2: cis-3-(3-((4-(benzyloxy)-6-fluoro-1,1-dioxido-2,3-dihydrobenzo[d]isothiazol-5-yl)amino)-1H-pyrazol-5-yl)cyclopentyl isopropylcarbamate

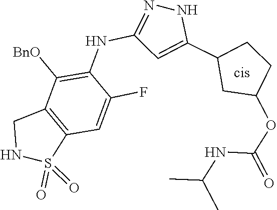 [SMILES: CC(C)NC(=O)OC1CCC(c2cc(Nc3c(F)cc4c(c3OCc3ccccc3)CNS4(=O)=O)n[nH]2)C1]

To a solution of 4-(benzyloxy)-5-bromo-6-fluoro-2,3-dihydrobenzo[d]isothiazole 1,1-dioxide (125 mg, 0.402 mmol) and cis-3-(3-amino-1H-pyrazol-5-yl)cyclopentyl isopropylcarbamate (100 mg, 0.402 mmol) in t-BuOH (5 mmol) was added BrettPhos Pd G3 (36 mg, 0.04 mmol) and K₂CO₃ (170 mg, 1.206 mmol), the reaction mixture was stirred for 3 hours at 105° C. under nitrogen. The mixture was cooled to room temperature, filtered, and the filtrate was concentrated under reduced pressure. The residue was purified by silica gel column chromatography, eluting with PE/EA (0%-100%) to afford the product (33 mg, 15%). LC-MS (ESI): m/z [M+H]⁺=544.1.

### Step 3: cis-3-(3-((6-fluoro-4-hydroxy-1,1-dioxido-2,3-dihydrobenzo[d]isothiazol-5-yl)amino)-1H-pyrazol-5-yl)cyclopentyl isopropylcarbamate

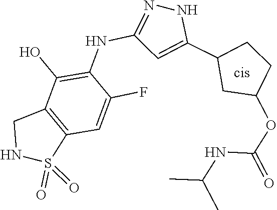 [SMILES: CC(C)NC(=O)OC1CCC(c2cc(Nc3c(F)cc4c(c3O)CNS4(=O)=O)n[nH]2)C1]

To a solution of cis-3-(3-((4-(benzyloxy)-6-fluoro-1,1-dioxido-2,3-dihydrobenzo[d]isothiazol-5-yl)amino)-1H-pyrazol-5-yl)cyclopentyl isopropylcarbamate (33 mg, 0.061 mmol) in THF/H₂O (5 mL/0.5 mL) was added Pd/C (10% on carbon, 10 mg), the reaction mixture was stirred for 3 hour under 1 atm hydrogen at room temperature. The resulting mixture was filtered, the filtrate was concentrated under reduce pressure, the residue was purified by prep-HPLC (SunFire Prep C18 column, eluting with 25%-40%-70% of water (containing 0.1% FA) in acetonitrile) to afford the product (3.5 mg, 13%). ¹H NMR (500 MHz, DMSO) δ=12.06 (s, 1H), 11.89 (s, 1H), 8.25 (s, 1H), 7.69 (t, J=4.9, 1H), 7.23 (d, J=8.4, 1H), 6.93 (d, J=7.6, 1H), 5.74 (s, 1H), 4.99 (s, 1H), 4.21 (d, J=4.8, 2H), 3.65-3.51 (m, 1H), 3.13-2.95 (m, 1H), 2.47-2.42 (m, 1H), 2.06-1.96 (m, 1H), 1.95-1.81 (m, 1H), 1.70 (m, 2H), 1.58 (m, 1H), 1.03 (m, 6H). LC-MS (ESI): m/z [M+H]⁺=454.29.

### Example 407: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl(3-methyltetrahydrofuran-3-yl)carbamate

### Step 1:5-bromo-4-fluoro-1-(2.2,2-trifluoroethyl)-1,3-dihydrobenzo[c]isothiazole 2,2-dioxide

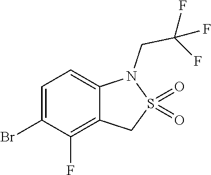 [SMILES: O=S1(=O)Cc2c(ccc(Br)c2F)N1CC(F)(F)F]

To a solution of 5-bromo-4-fluoro-1,3-dihydrobenzo[c]isothiazole 2,2-dioxide (1 g, 3.8 mmol) in DMF (40 mL) was added K₂CO₃ (781 mg, 5.7 mmol) and 2,2,2-Trifluoroethyl trifluoromethanesulfonate (881.6 mg, 3.8 mmol). The resulting solution was stirred at room temperature for 1 h. The solvent was concentrated, and the residue was purified by silica gel column chromatography, eluting with PE: EA=4:1 to afford the product (949 mg, 72% yield). LC-MS (ESI): m/z [M+Na]⁺=369.9.

### Step 2: cis-3-(3-amino-1H-pyrazol-5-yl)cyclopentyl(3-methyltetrahydrofuran-3-yl)carbamate

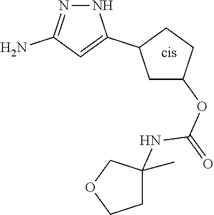 [SMILES: CC1(NC(=O)OC2CCC(c3cc(N)n[nH]3)C2)CCOC1]

The titled compound was synthesized in the procedures similar to example 34. LC-MS (ESI): m/z [M+H]⁺=295.2.

### Step 3: cis-3-(3-((4-fluoro-2,2-dioxido-1-(2.2,2-trifluoroethyl)-1,3-dihydrobenzo[c]isothiazol-5-yl)amino)-1H-pyrazol-5-yl)cyclopentyl(3-methyltetrahydrofuran-3-yl)carbamate

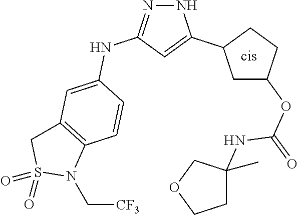 [SMILES: CC1(NC(=O)OC2CCC(c3cc(Nc4ccc5c(c4)CS(=O)(=O)N5CC(F)(F)F)n[nH]3)C2)CCOC1]

A mixture of 5-bromo-4-fluoro-1-(2,2,2-trifluoroethyl)-1,3-dihydrobenzo[c]isothiazole 2,2-dioxide (50 mg, 0.14 mmol), cis-3-(3-amino-1H-pyrazol-5-yl)cyclopentyl(3-methyltetrahydrofuran-3-yl)carbamate (42.2 mg, 0.14 mmol), Brettphos Pd G3 (12.6 mg, 0.014 mmol), K₂CO₃ (57.9 mg, 0.42 mmol) in t-BuOH (10 mL) was stirred at 110° C. for 16 h under N₂ atmosphere. The mixture was concentrated under reduced pressure. The residue was purified by silica gel column chromatography, eluting with EA to afford the product, which was further purified by prep HPLC (Waters XSelect C18: RD-CO-094 column, eluting with a gradient of acetonitrile/water containing 0.1% FA, 20%-40%) to afford the product (14.0 mg, 17.4%). ¹H NMR (500 MHz, DMSO-d₆) δ 11.77 (s, 1H), 8.03 (s, 2H), 7.23 (s, 1H), 6.83 (d, J=8.8 Hz, 1H), 5.70 (s, 1H), 4.99 (s, 1H), 4.86 (s, 2H), 4.43 (q, J=9.3 Hz, 2H), 3.82 (d, J=7.5 Hz, 1H), 3.79-3.65 (m, 2H), 3.43 (d, J=8.6 Hz, 1H), 3.09-2.89 (m, 1H), 2.47-2.40 (m, 1H), 2.20-2.10 (m, 1H), 2.05-1.98 (m, 1H), 1.96-1.84 (m, 1H), 1.82-1.65 (m, 3H), 1.65-1.55 (m, 1H), 1.32 (s, 3H). LC-MS (ESI): m/z [M+H]⁺=562.2.

### Example 408: cis-3-(3-((6-fluoro-2,2-dioxido-1,3-dihydrobenzo[c]isothiazol-5-yl)amino)-1H-pyrazol-5-yl)cyclopentyl(1-(hydroxymethyl)cyclobutyl)carbamate

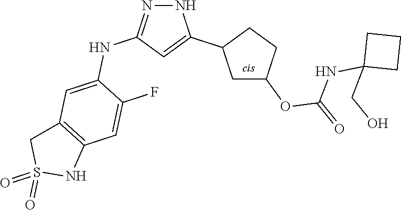 [SMILES: O=C(NC1(CO)CCC1)OC1CCC(c2cc(Nc3cc4c(cc3F)NS(=O)(=O)C4)n[nH]2)C1]

The titled compound was synthesized in the procedures similar to example 34. ¹H NMR (500 MHz, DMSO-d₆) δ 11.64 (s, 1H), 8.00 (s, 1H), 7.78 (s, 1H), 6.96 (s, 1H), 6.66 (d, J=11.5 Hz, 1H), 5.67 (s, 1H), 5.05-4.90 (m, 1H), 4.69 (t, J=5.8 Hz, 1H), 4.39 (s, 2H), 3.44 (d, J=5.3 Hz, 2H), 3.06-2.96 (m, 1H), 2.46-2.40 (m, 1H), 2.18-2.05 (m, 2H), 2.03-1.93 (m, 4H), 1.92-1.82 (m, 1H), 1.79-1.53 (m, 6H).

LC-MS (ESI): m/z [M+H]⁺=480.10.

### Example 409: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl(3-methyloxetan-3-yl)carbamate

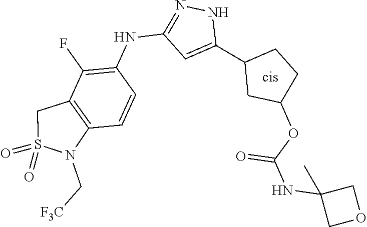 [SMILES: CC1(NC(=O)OC2CCC(c3cc(Nc4ccc5c(c4F)CS(=O)(=O)N5CC(F)(F)F)n[nH]3)C2)COC1]

The titled compound was synthesized in the procedures similar to Example 407.

¹H NMR (500 MHz, DMSO-d₆) δ 11.76 (s, 1H), 8.05 (s, 2H), 7.62 (s, 1H), 6.83 (d, J=8.7 Hz, 1H), 5.70 (s, 1H), 5.01 (s, 1H), 4.86 (s, 2H), 4.56 (s, 2H), 4.43 (dd, J=18.7, 9.2 Hz, 2H), 4.23 (s, 2H), 3.08-2.98 (m, 1H), 2.42 (s, 1H), 2.01 (d, J=8.9 Hz, 1H), 1.90 (s, 1H), 1.79-1.66 (m, 2H), 1.61 (s, 1H), 1.47 (s, 3H). LC-MS (ESI): m/z [M+H]⁺=548.2

### Example 410: (1s,3R)-3-(3-((6-fluoro-1,1-dioxido-2,3-dihydrobenzo[d]isothiazol-5-yl)amino)-1H-pyrazol-5-yl)cyclobutyl((S)-sec-butyl)carbamate

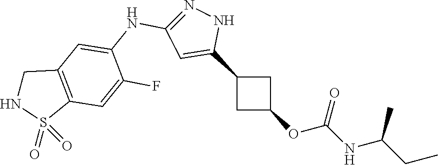 [SMILES: CC[C@H](C)NC(=O)O[C@H]1C[C@@H](c2cc(Nc3cc4c(cc3F)S(=O)(=O)NC4)n[nH]2)C1]

The titled compound was synthesized in the procedures similar to Example 50. ¹H NMR (500 MHz, DMSO-d₆) δ 12.07 (s, 1H), 8.76 (s, 1H), 8.13 (d, J=7.3 Hz, 1H), 7.70-7.59 (m, 2H), 7.00 (d, J=8.2 Hz, 1H), 5.88 (s, 1H), 4.89-4.76 (m, 1H), 4.30 (d, J=4.3 Hz, 2H), 3.41-3.36 (m, 1H), 3.13-3.03 (m, 1H), 2.72-2.62 (m, 2H), 2.13-2.04 (m, 2H), 1.41-1.32 (m, 2H), 1.01 (d, J=6.6 Hz, 3H), 0.81 (t, J=7.4 Hz, 3H). LC-MS (ESI): m/z [M+H]⁺=438.3.

### Example 411: (1s,3s)-3-(3-((6-fluoro-1,1-dioxido-2,3-dihydrobenzo[d]isothiazol-5-yl)amino)-1H-pyrazol-5-yl)cyclobutyl(1-methylcyclopropyl)carbamate

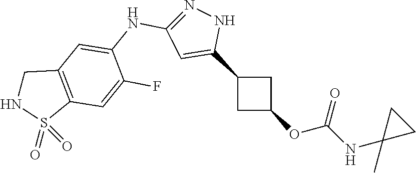 [SMILES: CC1(NC(=O)O[C@H]2C[C@@H](c3cc(Nc4cc5c(cc4F)S(=O)(=O)NC5)n[nH]3)C2)CC1]

The titled compound was synthesized in the procedures similar to Example 50. ¹H NMR (500 MHz, DMSO-d₆) δ 12.06 (s, 1H), 8.75 (s, 1H), 8.13 (s, 1H), 7.69-7.60 (m, 2H), 7.45 (s, 1H), 5.88 (s, 1H), 4.86-4.73 (m, 1H), 4.30 (d, J=4.9 Hz, 2H), 3.12-3.03 (m, 1H), 2.75-2.62 (m, 2H), 2.12-2.02 (m, 2H), 1.24 (s, 3H), 0.60 (s, 2H), 0.49 (s, 2H). LC-MS (ESI): m/z [M+H]⁺=436.3.

### Example 412: cis-3-(3-((6-fluoro-1,1-dioxido-2,3-dihydrobenzo[d]isothiazol-5-yl)amino)-1H-pyrazol-5-yl)cyclopentyl(4,4,4-trifluorobutan-2-yl)carbamate

### Step 1: cis-4-nitrophenyl 3-((6-fluoro-1,1-dioxido-2,3-dihydrobenzo[d]isothiazol-5-yl)amino)-5-(3-(((4-nitrophenoxy) carbonyl)oxy) cyclopentyl)-1H-pyrazole-1-carboxylate

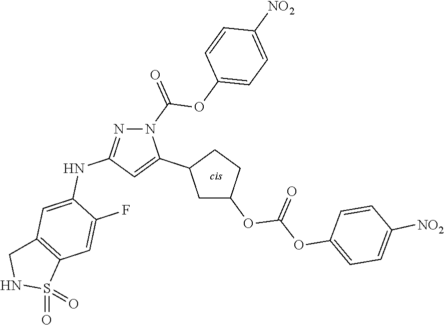 [SMILES: O=C(Oc1ccc([N+](=O)[O-])cc1)OC1CCC(c2cc(Nc3cc4c(cc3F)S(=O)(=O)NC4)nn2C(=O)Oc2ccc([N+](=O)[O-])cc2)C1]

The titled compound was synthesized in the procedures similar to Example 196. LC-MS (ESI): m/z [M+H]⁺=683.2.

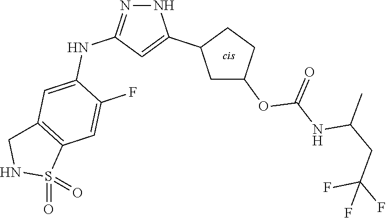 [SMILES: CC(CC(F)(F)F)NC(=O)OC1CCC(c2cc(Nc3cc4c(cc3F)S(=O)(=O)NC4)n[nH]2)C1]

The titled compound was synthesized in the procedures similar to Example 196. ¹H NMR (500 MHz, DMSO-d₆) δ 11.98 (s, 1H), 8.73 (s, 1H), 8.15 (d, J=7.3 Hz, 1H), 7.69-7.60 (m, 2H), 7.20 (s, 1H), 5.84 (s, 1H), 5.01 (s, 1H), 4.30 (d, J=4.9 Hz, 2H), 3.83 (s, 1H), 3.10-3.03 (m, 1H), 2.45-2.28 (m, 3H), 2.05-1.88 (m, 2H), 1.79-1.59 (m, 3H), 1.16-1.10 (m, 3H). LC-MS (ESI): m/z [M+H]⁺=506.3

### Example 413: cis-3-(3-((6-fluoro-1,1-dioxido-2,3-dihydrobenzo[d]isothiazol-5-yl)amino)-1H-pyrazol-5-yl)cyclopentyl(1-cyclopropylethyl)carbamate

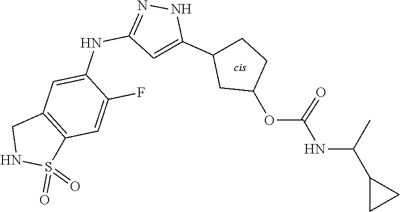 [SMILES: CC(NC(=O)OC1CCC(c2cc(Nc3cc4c(cc3F)S(=O)(=O)NC4)n[nH]2)C1)C1CC1]

The titled compound was synthesized in the procedures similar to Example 412. ¹H NMR (500 MHz, DMSO-d₆) δ 11.98 (s, 1H), 8.73 (s, 1H), 8.14 (s, 1H), 7.72-7.58 (m, 2H), 7.00 (d, J=8.0 Hz, 1H), 5.85 (s, 1H), 4.99 (s, 1H), 4.30 (d, J=4.8 Hz, 2H), 3.11-2.94 (m, 2H), 2.47-2.42 (m, 1H), 2.06-1.85 (m, 2H), 1.78-1.54 (m, 3H), 1.08 (d, J=6.2 Hz, 3H), 0.80 (s, 1H), 0.42-0.19 (m, 3H), 0.13-0.05 (m, 1H). LC-MS (ESI): m/z [M+H]⁺=464.3.

### Example 414: cis-3-(3-((4-fluoro-1,1-dioxido-2,3-dihydrobenzo[d]isothiazol-5-yl)amino)-1H-pyrazol-5-yl)cyclopentyl(cyclopropylmethyl)carbamate

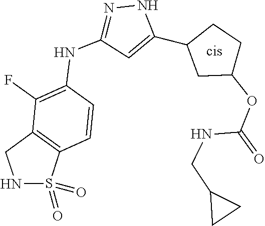 [SMILES: O=C(NCC1CC1)OC1CCC(c2cc(Nc3ccc4c(c3F)CNS4(=O)=O)n[nH]2)C1]

The titled compound was synthesized in the procedures similar to Example 328. ¹H NMR (500 MHz, DMSO-d₆) δ 11.98 (s, 1H), 8.71 (s, 1H), 8.26 (t, J=7.7, 1H), 7.69 (s, 1H), 7.48 (d, J=8.6, 1H), 7.13 (t, J=5.5, 1H), 5.83 (s, 1H), 5.01 (m, 1H), 4.41 (d, J=4.6, 2H), 3.12-3.02 (m, 1H), 2.85 (m, 2H), 2.48-2.43 (m, 1H), 2.09-1.99 (m, 1H), 1.98-1.84 (m, 1H), 1.81-1.66 (m, 2H), 1.61 (m, 1H), 0.88 (m, 1H), 0.42-0.30 (m, 2H), 0.13 (m, 2H). LC-MS (ESI): m/z [M+H]⁺=450.33.

### Example 415: cis-3-(3-((4-fluoro-2,2-dioxido-1,3-dihydrobenzo[c]isothiazol-5-yl)amino)-1H-pyrazol-5-yl)cyclopentyl morpholine-4-carboxylate

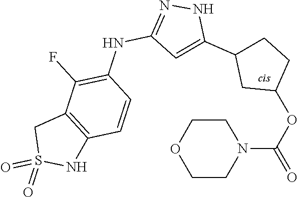 [SMILES: O=C(OC1CCC(c2cc(Nc3ccc4c(c3F)CS(=O)(=O)N4)n[nH]2)C1)N1CCOCC1]

The titled compound was synthesized in the procedures similar to Example 251. ¹H NMR (500 MHz, DMSO-d₆) δ 11.69 (s, 1H), 10.23 (s, 1H), 7.92 (s, 1H), 7.84 (s, 1H), 6.57 (d, J=8.6 Hz, 1H), 5.65 (s, 1H), 5.10-5.00 (dd, J=8.6, 4.6 Hz, 1H), 4.57 (s, 2H), 3.50 (s, 4H), 3.30-3.24 (m, 3H), 3.15-3.00 (m, 1H), 2.46-2.35 (m, 1H), 2.06-1.95 (m, 1H), 1.94-1.83 (m, 1H), 1.78-1.64 (m, 2H), 1.62-1.53 (m, 1H),LC-MS (ESI): m/z [M+H]⁺=466.2.

### Example 416: cis-3-(3-((4-fluoro-2,2-dioxido-1-(2,2,2-trifluoroethyl)-1,3-dihydrobenzo[c]isothiazol-5-yl)amino)-1H-pyrazol-5-yl)cyclopentyl(1-methoxy-2-methylpropan-2-yl)carbamate

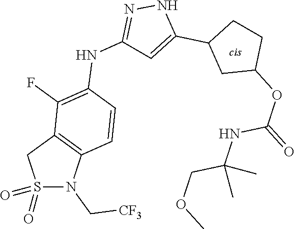 [SMILES: COCC(C)(C)NC(=O)OC1CCC(c2cc(Nc3ccc4c(c3F)CS(=O)(=O)N4CC(F)(F)F)n[nH]2)C1]

The titled compound was synthesized in the procedures similar to Example 409. ¹H NMR (500 MHz, DMSO-d₆) δ 11.74 (s, 1H), 8.06 (d, J=19.1 Hz, 2H), 6.83 (d, J=8.8 Hz, 1H), 6.63 (s, 1H), 5.70 (s, 1H), 4.97 (s, 1H), 4.86 (s, 2H), 4.43 (q, J=9.3 Hz, 2H), 3.24 (s, 3H), 3.09-2.93 (m, 1H), 2.46-2.35 (m, 1H), 2.06-1.95 (m, 1H), 1.94-1.83 (m, 1H), 1.78-1.64 (m, 2H), 1.62-1.53 (m, 1H), 1.16 (s, 6H).

LC-MS (ESI): m/z [M+H]⁺=564.2.

### Example 417: cis-3-(3-((2,2-dioxido-1,3-dihydrobenzo[c]isothiazol-5-yl)amino)-1H-pyrazol-5-yl)cyclopentyl(1-(hydroxymethyl)cyclobutyl)carbamate

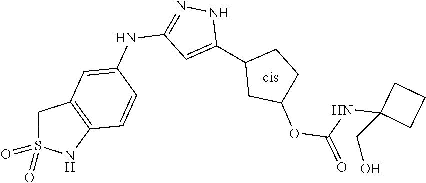 [SMILES: O=C(NC1(CO)CCC1)OC1CCC(c2cc(Nc3ccc4c(c3)CS(=O)(=O)N4)n[nH]2)C1]

The titled compound was synthesized in the procedures similar to Example 343. ¹H NMR (500 MHz, DMSO-d₆) δ 11.62 (s, 1H), 9.74 (s, 1H), 8.19 (s, 1H), 7.37 (s, 1H), 7.15 (d, J=8.0 Hz, 1H), 6.96 (s, 1H), 6.71 (d, J=8.6 Hz, 1H), 5.58 (s, 1H), 5.05-4.90 (m, 1H), 4.70 (t, J=5.8 Hz, 1H), 4.44 (s, 2H), 3.44 (d, J=5.4 Hz, 2H), 3.06-2.96 (m, 1H), 2.48-2.40 (m, 1H), 2.16-2.05 (m, 2H), 2.04-1.93 (m, 3H), 1.91-1.83 (m, 1H), 1.78-1.56 (m, 5H). LC-MS (ESI): m/z [M+H]⁺=462.35.

### Example 418: cis-3-(3-((4-fluoro-2,2-dioxido-1,3-dihydrobenzo[c]isothiazol-5-yl)amino)-1H-pyrazol-5-yl)cyclopentyl(1-(hydroxymethyl)cyclobutyl)carbamate

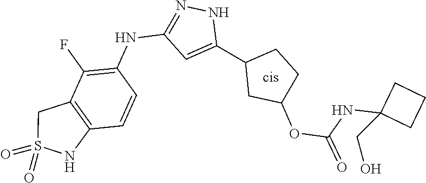 [SMILES: O=C(NC1(CO)CCC1)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.65 (s, 1H), 10.29 (s, 1H), 7.88 (s, 1H), 7.79 (s, 1H), 6.96 (s, 1H), 6.55 (d, J=8.6 Hz, 1H), 5.66 (s, 1H), 4.97 (s, 1H), 4.69 (s, 1H), 4.53 (s, 2H), 3.44 (d, J=5.4 Hz, 2H), 3.06-2.96 (m, 1H), 2.48-2.40 (m, 1H), 2.16-2.05 (m, 2H), 2.04-1.93 (m, 3H), 1.91-1.83 (m, 1H), 1.78-1.56 (m, 5H).

LC-MS (ESI): m/z [M+H]⁺=480.2.

### Example 419: cis-3-(3-((6-fluoro-1,1-dioxido-2,3-dihydrobenzo[d]isothiazol-5-yl)amino)-1H-pyrazol-5-yl)cyclopentyl(3,3,3-trifluoropropyl)carbamate

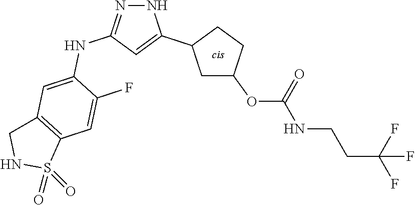 [SMILES: O=C(NCCC(F)(F)F)OC1CCC(c2cc(Nc3cc4c(cc3F)S(=O)(=O)NC4)n[nH]2)C1]

The titled compound was synthesized in the procedures similar to Example 412 in a racemic form, which was further separated by Chiral Prep-HPLC to give:

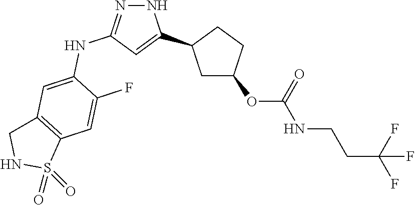 [SMILES: O=C(NCCC(F)(F)F)O[C@@H]1CC[C@H](c2cc(Nc3cc4c(cc3F)S(=O)(=O)NC4)n[nH]2)C1]

Enantiomer 1 (Example 419a, (1R,3S)-3-(3-((6-fluoro-1,1-dioxido-2,3-dihydrobenzo[d]isothiazol-5-yl)amino)-1H-pyrazol-5-yl)cyclopentyl(3,3,3-trifluoropropyl)carbamate, 100% ee); Retention time: 5.803 min. ¹H NMR (500 MHz, DMSO-d₆) δ 11.98 (s, 1H), 8.73 (s, 1H), 8.16 (s, 1H), 7.72-7.59 (m, 2H), 7.26 (d, J=5.5 Hz, 1H), 5.84 (s, 1H), 5.02 (s, 1H), 4.30 (d, J=4.9 Hz, 2H), 3.21 (dd, J=12.9, 6.6 Hz, 2H), 3.12-3.04 (m, 1H), 2.47-2.35 (m, 3H), 2.06-1.86 (m, 2H), 1.77-1.57 (m, 3H). LC-MS (ESI): m/z [M+H]⁺=492.2.

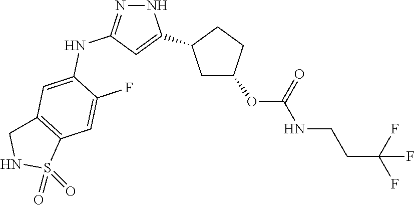 [SMILES: O=C(NCCC(F)(F)F)O[C@H]1CC[C@@H](c2cc(Nc3cc4c(cc3F)S(=O)(=O)NC4)n[nH]2)C1]

Enantiomer 2 (Example 419b, (1S,3R)-3-(3-((6-fluoro-1,1-dioxido-2,3-dihydrobenzo[d]isothiazol-5-yl)amino)-1H-pyrazol-5-yl)cyclopentyl(3,3,3-trifluoropropyl)carbamate, 100% ee); Retention time: 6.884 min. ¹H NMR (500 MHz, DMSO-d₆) δ 11.98 (s, 1H), 8.73 (s, 1H), 8.16 (s, 1H), 7.72-7.59 (m, 2H), 7.26 (d, J=5.5 Hz, 1H), 5.84 (s, 1H), 5.02 (s, 1H), 4.30 (d, J=4.9 Hz, 2H), 3.21 (dd, J=12.9, 6.6 Hz, 2H), 3.12-3.04 (m, 1H), 2.47-2.35 (m, 3H), 2.06-1.86 (m, 2H), 1.77-1.57 (m, 3H). LC-MS (ESI): m/z [M+H]⁺=492.2.

Chiral analytical method: Column: CHIRALPAK IE 4.6 mm×250 mm, 5 μm; Mobile phase: A for Hexane and B for ETOH (0.1% 2M NH₃ MeOH); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for ETOH (0.1% 2M NH₃ MeOH); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 420: cis-3-(3-((6-fluoro-1,1-dioxido-2,3-dihydrobenzo[d]isothiazol-5-yl)amino)-1H-pyrazol-5-yl)cyclopentyl(cyclopropylmethyl)carbamate

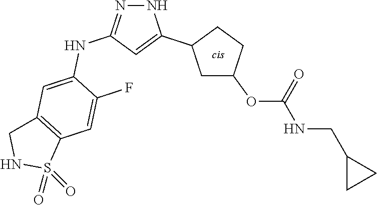 [SMILES: O=C(NCC1CC1)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 11.98 (s, 1H), 8.73 (s, 1H), 8.15 (d, J=7.3 Hz, 1H), 7.70-7.60 (m, 2H), 7.14 (d, J=5.5 Hz, 1H), 5.85 (s, 1H), 5.01 (s, 1H), 4.30 (d, J=4.8 Hz, 2H), 3.12-3.05 (m, 1H), 2.85 (t, J=6.2 Hz, 2H), 2.47-2.44 (m, 1H), 2.08-1.86 (m, 2H), 1.78-1.56 (m, 3H), 0.88 (s, 1H), 0.37 (d, J=7.0 Hz, 2H), 0.13 (d, J=4.5 Hz, 2H). LC-MS (ESI): m/z [M+H]⁺=450.3.

### Example 421: cis-3-(3-((6-fluoro-1,1-dioxido-2,3-dihydrobenzo[d]isothiazol-5-yl)amino)-1H-pyrazol-5-yl)cyclopentyl ethylcarbamate

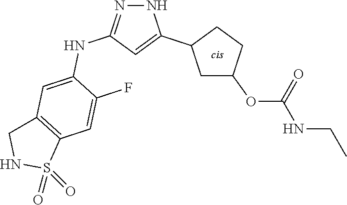 [SMILES: CCNC(=O)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 11.98 (s, 1H), 8.73 (s, 1H), 8.15 (d, J=6.7 Hz, 1H), 7.68-7.59 (m, 2H), 7.02 (s, 1H), 5.85 (s, 1H), 5.01 (s, 1H), 4.30 (d, J=4.9 Hz, 2H), 3.12-3.03 (m, 1H), 3.02-2.95 (m, 2H), 2.48-2.42 (m, 1H), 2.06-1.85 (m, 2H), 1.78-1.55 (m, 3H), 0.99 (t, J=7.2 Hz, 3H). LC-MS (ESI): m/z [M+H]⁺=424.3.

### Example 422: cis-3-(3-((6-fluoro-1,1-dioxido-2,3-dihydrobenzo[d]isothiazol-5-yl)amino)-1H-pyrazol-5-yl)cyclopentyl bicyclo[1.1.1]pentan-1-ylcarbamate

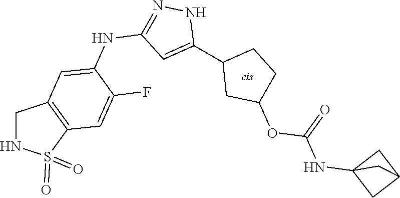 [SMILES: O=C(NC12CC(C1)C2)OC1CCC(c2cc(Nc3cc4c(cc3F)S(=O)(=O)NC4)n[nH]2)C1]

The titled compound was synthesized in the procedures similar to Example 412 in a racemic form, which was further separated by Chiral Prep-HPLC to give:

Enantiomer 1 (Example 422a, 100% ee); Retention time: 7.718 min. ¹H NMR (500 MHz, DMSO-d₆)) δ 11.97 (s, 1H), 8.74 (s, 1H), 8.15 (d, J=7.3 Hz, 1H), 7.76 (s, 1H), 7.68-7.60 (m, 2H), 5.85 (s, 1H), 5.00 (s, 1H), 4.30 (d, J=4.4 Hz, 2H), 3.13-3.01 (m, 1H), 2.48-2.43 (m, 1H), 2.09-1.98 (m, 1H), 1.95-1.82 (m, 7H), 1.79-1.52 (m, 3H). LC-MS (ESI): m/z [M+H]⁺=464.3.

Enantiomer 2 (Example 422b, 97.8% ee); Retention time: 9.266 min. ¹H NMR (500 MHz, DMSO-d₆)) δ 11.97 (s, 1H), 8.74 (s, 1H), 8.15 (d, J=7.3 Hz, 1H), 7.76 (s, 1H), 7.68-7.60 (m, 2H), 5.85 (s, 1H), 5.00 (s, 1H), 4.30 (d, J=4.4 Hz, 2H), 3.13-3.01 (m, 1H), 2.48-2.43 (m, 1H), 2.09-1.98 (m, 1H), 1.95-1.82 (m, 7H), 1.79-1.52 (m, 3H). LC-MS (ESI): m/z [M+H]⁺=464.3.

Chiral analytical method: Column: CHIRALPAK IE 4.6 mm×250 mm, 5 μm; Mobile phase: A for Hexane and B for ETOH (0.1% 2M NH₃ MeOH); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for ETOH (0.1% 2M NH₃ MeOH); Gradient: Mobile Phase A: Mobile Phase B=50: 50 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 423: cis-3-(3-((4-fluoro-2,2-dioxido-1,3-dihydrobenzo[c]isothiazol-5-yl)amino)-1H-pyrazol-5-yl)cyclopentyl tert-butylcarbamate

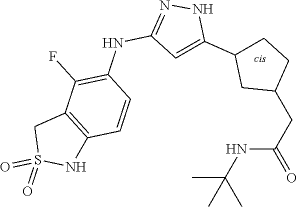 [SMILES: CC(C)(C)NC(=O)CC1CCC(c2cc(Nc3ccc4c(c3F)CS(=O)(=O)N4)n[nH]2)C1]

The titled compound was synthesized in the procedures similar to Example 245. ¹H NMR (500 MHz, DMSO-d₆) δ 11.68 (s, 1H), 10.28 (s, 1H), 7.91 (t, J=5 Hz, 1H), 7.85 (s, 1H), 6.76 (s, 1H), 6.58 (d, J=10 Hz, 1H), 5.67 (s, 1H), 5.01-4.93 (m, 1H), 4.58 (s, 2H), 3.05-2.96 (m, 1H), 2.48-2.40 (m, 1H), 2.04-1.95 (m, 1H), 1.93-1.83 (m, 1H), 1.75-1.63 (m, 2H), 1.61-1.52 (m, 1H), 1.20 (s, 9H). LC-MS (ESI): m/z [M+H]⁺=452.2.

### Example 424: cis-3-(3-((1-methyl-2,2-dioxido-1,3-dihydrobenzo[c]isothiazol-5-yl)amino)-1H-pyrazol-5-yl)cyclopentyl(tetrahydrofuran-3-yl)carbamate

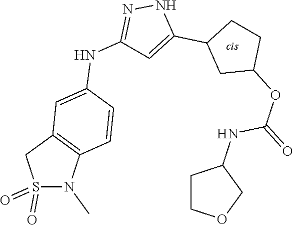 [SMILES: CN1c2ccc(Nc3cc(C4CCC(OC(=O)NC5CCOC5)C4)[nH]n3)cc2CS1(=O)=O]

The titled compound was synthesized in the procedures similar to Example 324. ¹H NMR (500 MHz, DMSO-d₆) δ 11.63 (s, 1H), 8.22 (s, 1H), 7.41 (s, 1H), 7.35 (d, J=6.1 Hz, 1H), 7.26 (d, J=7.5 Hz, 1H), 6.78 (d, J=8.7 Hz, 1H), 5.57 (s, 1H), 5.01 (s, 1H), 4.56 (s, 2H), 4.02 (s, 1H), 3.78-3.69 (m, 2H), 3.69-3.60 (m, 1H), 3.47-3.40 (m, 1H), 3.09-2.99 (m, 1H), 2.95 (s, 3H), 2.44-2.35 (m, 1H), 2.05-1.95 (m, 2H), 1.95-1.85 (m, 1H), 1.80-1.65 (m, 3H), 1.65-1.55 (m, 1H). LC-MS (ESI): m/z [M+H]⁺=462.2.

### Example 425: cis-3-(3-((1,1-dioxido-2,3-dihydroisothiazolo[5,4-c]pyridin-5-yl)amino)-1H-pyrazol-5-yl)cyclopentyl isopropylcarbamate

### Step 1:5-bromo-2,3-dihydroisothiazolo[5,4-c]pyridine 1,1-dioxide

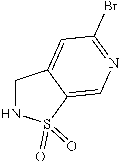 [SMILES: O=S1(=O)NCc2cc(Br)ncc21]

The titled compound was synthesized in the procedures similar to Example 1. LC-MS (ESI): m/z [M+H]⁺=248.9.

### Step 1: cis-3-(3-((1,1-dioxido-2,3-dihydroisothiazolo[5,4-c]pyridin-5-yl)amino)-1H-pyrazol-5-yl)cyclopentyl isopropylcarbamate

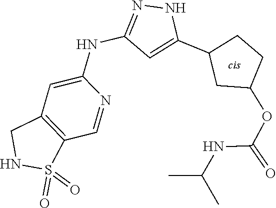 [SMILES: CC(C)NC(=O)OC1CCC(c2cc(Nc3cc4c(cn3)S(=O)(=O)NC4)n[nH]2)C1]

A mixture of 5-bromo-2,3-dihydroisothiazolo[5,4-c]pyridine 1,1-dioxide (50 mg, 0.20 mmol), cis-3-(3-amino-1H-pyrazol-5-yl)cyclopentyl isopropylcarbamate (50.4 mg, 0.20 mmol), Pd₂(dba)₃ (18.2 mg, 0.02 mmol), Xantphos (21.8 mg, 0.04 mmol), K₃PO₄ (127.2 mg, 0.60 mmol) in dioxane (10 mL) was stirred at 90° C. for 16 h under N₂ atmosphere. The mixture was concentrated under reduced pressure. The residue was purified by silica gel column chromatography, eluting with EA to afford the product 20 mg. The residue 50 mg was purified by prep HPLC (Waters XSelect C18: RD-CO-094 column, eluting with a gradient of acetonitrile/water containing 0.1% FA, 20%-40%) to afford the product (2.5 mg, 2.9%). 1H NMR (500 MHz, DMSO-d₆) δ 8.89 (s, 1H), 7.98 (s, 1H), 7.93 (s, 1H), 6.93 (d, J=7.8 Hz, 1H), 6.84 (s, 2H), 5.32 (s, 1H), 4.99 (s, 1H), 4.51 (s, 2H), 3.58 (d, J=7.1 Hz, 1H), 2.95 (d, J=9.0 Hz, 1H), 2.44-2.35 (m, 1H), 2.00-1.92 (m, 1H), 1.92-1.84 (m, 1H), 1.82-1.68 (m, 3H), 1.03 (d, J=6.2 Hz, 6H). LC-MS (ESI): m/z [M+H]⁺=421.2.

### Example 426: cis-3-(3-((1-methyl-2,2-dioxido-1,3-dihydrobenzo[c]isothiazol-5-yl)amino)-1H-pyrazol-5-yl)cyclopentyl((1-hydroxycyclopropyl)methyl)carbamate

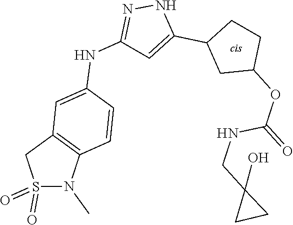 [SMILES: CN1c2ccc(Nc3cc(C4CCC(OC(=O)NCC5(O)CC5)C4)[nH]n3)cc2CS1(=O)=O]

The titled compound was synthesized in the procedures similar to Example 324. ¹H NMR (500 MHz, DMSO-d₆) δ 11.63 (s, 1H), 8.21 (s, 1H), 7.42 (s, 1H), 7.26 (d, J=7.0 Hz, 1H), 7.00 (t, J=5.8 Hz, 1H), 6.78 (d, J=8.6 Hz, 1H), 5.58 (s, 1H), 5.25 (s, 1H), 5.00 (s, 1H), 4.56 (s, 2H), 3.12 (d, J=5.9 Hz, 2H), 3.08-3.00 (m, 1H), 2.95 (s, 3H), 2.46-2.35 (m, 1H), 2.06-1.95 (m, 1H), 1.94-1.83 (m, 1H), 1.78-1.64 (m, 2H), 1.62-1.53 (m, 1H), 0.53-0.39 (m, 4H). LC-MS (ESI): m/z [M+H]⁺=462.2.

### Example 427: cis-3-(3-((4-fluoro-2,2-dioxido-1,3-dihydrobenzo[c]isothiazol-5-yl)amino)-1H-pyrazol-5-yl)cyclopentyl(1-methylcyclobutyl)carbamate

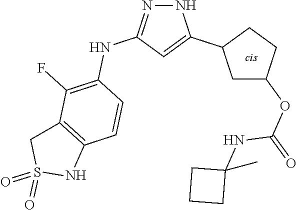 [SMILES: CC1(NC(=O)OC2CCC(c3cc(Nc4ccc5c(c4F)CS(=O)(=O)N5)n[nH]3)C2)CCC1]

The titled compound was synthesized in the procedures similar to Example 251 in a racemic form, which was separated by Chiral Prep-HPLC to give:

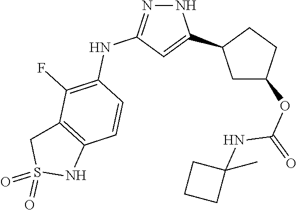 [SMILES: CC1(NC(=O)O[C@@H]2CC[C@H](c3cc(Nc4ccc5c(c4F)CS(=O)(=O)N5)n[nH]3)C2)CCC1]

Enantiomer 1 (Example 427a, (1R,3S)-3-(3-((4-fluoro-2,2-dioxido-1,3-dihydrobenzo[c]isothiazol-5-yl)amino)-1H-pyrazol-5-yl)cyclopentyl(1-methylcyclobutyl)carbamate, 100% ee); Retention time: 4.364 min. ¹H NMR (500 MHz, DMSO-d₆) δ 11.67 (s, 1H), 10.20 (s, 1H), 7.94 (t, J=9.1 Hz, 1H), 7.86 (s, 1H), 7.16 (s, 1H), 6.58 (d, J=8.7 Hz, 1H), 5.67 (s, 1H), 4.97 (s, 1H), 4.58 (s, 2H), 3.06-2.97 (m, 1H), 2.48-2.42 (m, 1H), 2.26-2.15 (m, 2H), 2.04-1.85 (m, 2H), 1.83-1.64 (m, 6H), 1.63-1.53 (m, 1H), 1.31 (s, 3H). LC-MS (ESI): m/z [M+H]⁺=464.3.

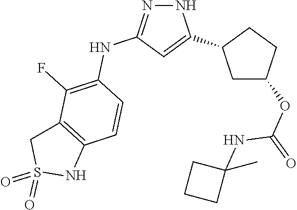 [SMILES: CC1(NC(=O)O[C@H]2CC[C@@H](c3cc(Nc4ccc5c(c4F)CS(=O)(=O)N5)n[nH]3)C2)CCC1]

Enantiomer 2 (Example 427b, (1S,3R)-3-(3-((4-fluoro-2,2-dioxido-1,3-dihydrobenzo[c]isothiazol-5-yl)amino)-1H-pyrazol-5-yl)cyclopentyl(1-methylcyclobutyl)carbamate, 100% ee); Retention time: 6.886 min. ¹H NMR (500 MHz, DMSO-d₆) δ 11.67 (s, 1H), 10.20 (s, 1H), 7.94 (t, J=9.1 Hz, 1H), 7.86 (s, 1H), 7.16 (s, 1H), 6.58 (d, J=8.7 Hz, 1H), 5.67 (s, 1H), 4.97 (s, 1H), 4.58 (s, 2H), 3.06-2.97 (m, 1H), 2.48-2.42 (m, 1H), 2.26-2.15 (m, 2H), 2.04-1.85 (m, 2H), 1.83-1.64 (m, 6H), 1.63-1.53 (m, 1H), 1.31 (s, 3H). LC-MS (ESI): m/z [M+H]⁺=464.3.

Chiral analytical method: Column: CHIRALPAK IE 4.6 mm×250 mm, 5 μm; Mobile phase: A for MTBE (0.1% 2M NH₃ MeOH) and B for MEOH/DCM (50/50); Gradient: Mobile Phase A: Mobile Phase B=60: 40 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE (0.1% 2M NH₃ MeOH) and B for MEOH/DCM (50/50); Gradient: Mobile Phase A: Mobile Phase B=60: 40 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 428: cis-3-(3-((2,2-dioxido-1,3-dihydrobenzo[c]isothiazol-5-yl)amino)-1H-pyrazol-5-yl)cyclopentyl(tetrahydrofuran-3-yl)carbamate

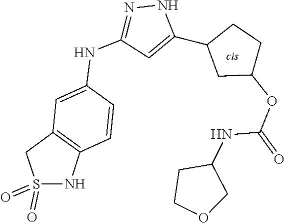 [SMILES: O=C(NC1CCOC1)OC1CCC(c2cc(Nc3ccc4c(c3)CS(=O)(=O)N4)n[nH]2)C1]

The titled compound was synthesized in the procedures similar to Example 343. ¹H NMR (500 MHz, DMSO-d₆) δ 11.62 (s, 1H), 9.74 (s, 1H), 8.19 (s, 1H), 7.44-7.29 (m, 2H), 7.15 (s, 1H), 6.71 (d, J=8.6 Hz, 1H), 5.57 (s, 1H), 5.01 (s, 1H), 4.44 (s, 2H), 4.02 (s, 1H), 3.81-3.67 (m, 2H), 3.68-3.60 (m, 1H), 3.46-3.40 (m, 1H), 3.02 (s, 1H), 2.44-2.35 (m, 1H), 2.08-1.96 (m, 2H), 1.94-1.84 (m, 1H), 1.80-1.66 (m, 3H), 1.64-1.54 (m, 1H). LC-MS (ESI): m/z [M+H]⁺=448.2.

### Example 429: cis-3-(3-((1-cyclopropyl-4-fluoro-2,2-dioxido-1,3-dihydrobenzo[c]isothiazol-5-yl)amino)-1H-pyrazol-5-yl)cyclopentyl(tetrahydrofuran-3-yl)carbamate

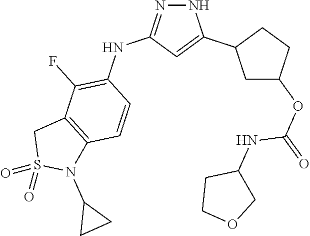 [SMILES: O=C(NC1CCOC1)OC1CCC(c2cc(Nc3ccc4c(c3F)CS(=O)(=O)N4C3CC3)n[nH]2)C1]

The titled compound was synthesized in the procedures similar to Example 363. ¹H NMR (500 MHz, DMSO-d₆) δ 11.74 (s, 1H), 8.02 (s, 1H), 7.93 (s, 1H), 7.34 (d, J=5.9 Hz, 1H), 6.83 (d, J=8.7 Hz, 1H), 5.68 (s, 1H), 5.01 (s, 1H), 4.70 (s, 2H), 4.02 (s, 1H), 3.77-3.69 (m, 2H), 3.68-3.60 (m, 1H), 3.47-3.40 (m, 1H), 3.11-2.97 (m, 1H), 2.47-2.41 (m, 1H), 2.08-1.96 (m, 2H), 1.94-1.84 (m, 1H), 1.80-1.66 (m, 3H), 1.64-1.54 (m, 1H), 0.98-0.91 (m, 2H), 0.90-0.83 (m, 2H). LC-MS (ESI): m/z [M+H]⁺=506.2.

### Example 430: cis-3-(3-((5-fluoro-2,2-dioxido-3,4-dihydro-1H-benzo[c][1,2]thiazin-6-yl)amino)-1H-pyrazol-5-yl)cyclopentyl isopropylcarbamate

### Step 1:1,4-dibromo-2-fluoro-3-(2-methoxyvinyl)benzene

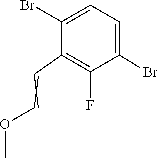

To a solution of (methoxymethyl)triphenylphosphonium chloride (20.7 g, 60.4 mmol) in THF (150 mL) was added KHMDS (71 mL, 71 mmol, 1M) at 0° C. The resulting solution was stirred for 30 min at 0° C. Then the solution of 3,6-dibromo-2-fluorobenzaldehyde (10 g, 35.5 mmol) in THF (20 mL) was added to the reaction at 0° C. The resulting solution was stirred for 2 h at rt under nitrogen atmosphere. The reaction was quenched by the addition of sat.aq. NH₄Cl solution. The resulting solution was extracted with 3×150 mL of EtOAc. The resulting mixture was concentrated under vacuum. The residue was purified by combi-flash PE to give the product (7.7 g, 70%) as yellow oil. LCMS (ESI) m/z [M+H]⁺=311.

### Step 2:2-(3,6-dibromo-2-fluorophenyl) acetaldehyde

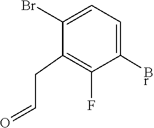 [SMILES: O=CCc1c(Br)ccc(Br)c1F]

The solution of 1,4-dibromo-2-fluoro-3-(2-methoxyvinyl)benzene (7.7 g, 24.8 mmol) in HCl-dioxane (70 mL) was stirred for 1 h at rt. The resulting mixture was concentrated under vacuum. The residue was purified by combi-flash (EtOAc/PE=4%) to give the product (5.6 g, 76.7%) as yellow oil.

LCMS (ESI) m/z [M+H]⁺=297.

### Step 3:2-(3,6-dibromo-2-fluorophenyl)ethan-1-ol

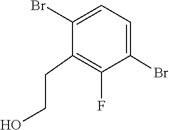 [SMILES: OCCc1c(Br)ccc(Br)c1F]

To a solution of 2-(3,6-dibromo-2-fluorophenyl) acetaldehyde (5.6 g, 19 mmol) in THF (80 mL) was added NaBH₄ (1.4 g, 38 mmol) at 0° C. The resulting solution was stirred for 2 h at rt. The reaction was then quenched by the addition of MeOH. The resulting mixture was concentrated under vacuum. The residue was purified by combi-flash (EtOAc/PE=12%-40%) to give the product (3.1 g, 54.4% %) as colorless oil. LCMS (ESI) m/z [M+H]⁺=299.

### Step 4:1,4-dibromo-2-(2-bromoethyl)-3-fluorobenzene

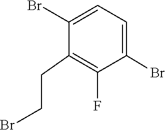 [SMILES: Fc1c(Br)ccc(Br)c1CCBr]

To a solution of 2-(3,6-dibromo-2-fluorophenyl)ethan-1-ol (3.1, 10.4 mmol) in DCM (40 mL) was added PPh₃ (3.5 g, 13.5 mmol). Then the solution of CBr₄ (5.8 g, 17.6 mmol) in DCM (10 mL) was added to the reaction at 0° C. The resulting solution was stirred for 3 h at rt under nitrogen atmosphere. The resulting solution was extracted with 3×30 mL of DCM. The resulting mixture was concentrated under vacuum. The residue was purified by combi-flash PE to give the product (3.7 g, 97.4%) as colorless oil. LCMS (ESI) m/z [M+H]⁺=361.

### Step 5: S-(3,6-dibromo-2-fluorophenethyl)ethanethioate

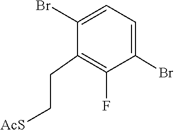

To a solution of 1,4-dibromo-2-(2-bromoethyl)-3-fluorobenzene (3.7 g, 10.2 mmol) in DMF (35 mL) was added potassium ethanethioate (2.3 g, 20.4 mmol) at 0° C. The resulting solution was stirred for 2 h at rt. The resulting solution was extracted with 3×50 mL of EtOAc. The resulting mixture was concentrated under vacuum. The residue was the crude product (3 g, 83.3%). LCMS (ESI) m/z [M+H]⁺=357.

### Step 6:2-(3,6-dibromo-2-fluorophenyl)ethane-1-sulfonyl chloride

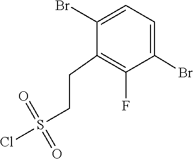 [SMILES: O=S(=O)(Cl)CCc1c(Br)ccc(Br)c1F]

To a solution of NCS (4.5 g, 33.6 mmol) in MeCN (30 mL) and HCl (8 mL, 2M) was added the solution of S-(3,6-dibromo-2-fluorophenethyl)ethanethioate (3 g, 8.4 mmol) in MeCN (10 mL) at 0° C. The resulting solution was stirred for 30 min at 0° C. The resulting solution was extracted with 3×50 mL of EtOAc. The resulting mixture was concentrated under vacuum. The residue was the crude product (3.2 g, 100%). LCMS (ESI) m/z [M+H]⁺=381.

### Step 7:2-(3,6-dibromo-2-fluorophenyl)ethane-1-sulfonamide

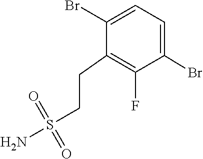 [SMILES: NS(=O)(=O)CCc1c(Br)ccc(Br)c1F]

To a solution of 2-(3,6-dibromo-2-fluorophenyl)ethane-1-sulfonyl chloride (3.2 g, 8.4 mmol) in THF (40 mL) was added NH₃·H₂O (10 mL) at 0° C. The resulting solution was stirred for 1 h at rt. The resulting mixture was concentrated under vacuum. The residue was purified by combi-flash (MeOH/DCM=31%) to give the product (1.5 g, 46.9%). LCMS (ESI) m/z [M+H]⁺=362.

### Step 8:6-bromo-5-fluoro-3,4-dihydro-1H-benzo[c] [1.2]thiazine 2,2-dioxide

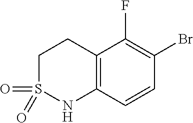 [SMILES: O=S1(=O)CCc2c(ccc(Br)c2F)N1]

To a solution of 2-(3,6-dibromo-2-fluorophenyl)ethane-1-sulfonamide (1.5 g, 4.2 mmol) in DMF (15 mL) were added CuI (223 mg, 2.5 mmol), methylglycine (27 mg, 1.3 mmol), K₃PO₄ (4.5 g, 21 mmol). The resulting solution was stirred for 1 h at 80° C. under nitrogen atmosphere. The resulting solution was extracted with 3×50 mL of EtOAc. The resulting mixture was concentrated under vacuum. The residue was purified by combi-flash (MeOH/DCM=0%-11%) to give the product (315 mg, 24.2%).

LC-MS (ESI): m/z [M+H]⁺=279.9.

### Step 9: 6-bromo-5-fluoro-1-(4-methoxybenzyl)-3,4-dihydro-1H-benzo [c][1 2]thiazine 2,2-dioxide

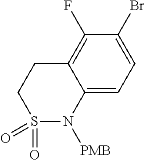

To a stirring mixture of 6-bromo-5-fluoro-3,4-dihydro-1H-benzo[c][1,2]thiazine 2,2-dioxide (280 mg, 1 mmol) and K₂CO₃ (276 mg, 2 mmol) in DMF (5 mL) was added PMBBr (211 mg, 1.05 mmol) dropwise at 0° C. The solution was warmed to room temperature and stirred under nitrogen atmosphere for 1 h. The mixture was concentrated under reduced pressure, and the residue was purified by silica gel column chromatography, eluting with PE/EA (10:1) to afford the product (320 mg, 80%).

LC-MS (ESI): m/z [M+Na]⁺=422.1.

### Step 10: cis-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl isopropylcarbamate

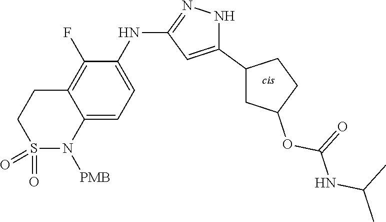

To a mixture of 6-bromo-5-fluoro-1-(4-methoxybenzyl)-3,4-dihydro-1H-benzo[c][1,2]thiazine 2,2-dioxide (320 mg, 0.8 mmol) and cis-3-(3-amino-1H-pyrazol-5-yl)cyclopentyl isopropylcarbamate (201 mg, 0.8 mmol) in t-BuOH (3 mL) was added BrettPhos Pd G3 (72 mg, 0.08 mmol) and K₂CO₃ (331 mg, 2.4 mmol). The reaction mixture was stirred at 110° C. under nitrogen atmosphere for 4 h. The mixture was concentrated under reduced pressure. The residue was purified by silica gel column chromatography, eluting with PE/EA (1:9) to afford the product (398 mg, 87%). LC-MS (ESI): m/z [M+H]⁺=572.3.

### Step 11: cis-3-(3-((5-fluoro-2,2-dioxido-3,4-dihydro-1H-benzo [c][1 2]thiazin-6-yl)amino)-1H-pyrazol-5-yl)cyclopentyl isopropylcarbamate

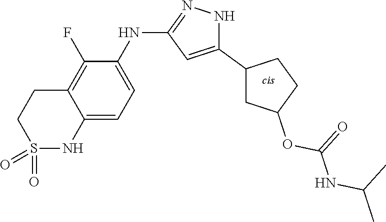 [SMILES: CC(C)NC(=O)OC1CCC(c2cc(Nc3ccc4c(c3F)CCS(=O)(=O)N4)n[nH]2)C1]

A mixture of cis-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl isopropylcarbamate (398 mg, 0.69 mmol and TFA (2 mL) in DCM (2 mL) was stirred at RT for 12 h. The mixture was concentrated under reduced pressure. The residue was purified by silica gel column chromatography, eluting with DCM/MeOH (20:1) to afford the product (300 mg, 95%) in a racemic form, which was further separated by Chiral Prep-HPLC to give:

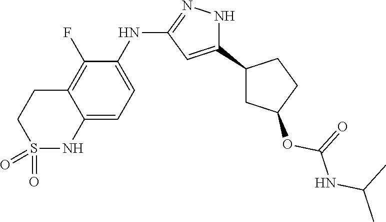 [SMILES: CC(C)NC(=O)O[C@@H]1CC[C@H](c2cc(Nc3ccc4c(c3F)CCS(=O)(=O)N4)n[nH]2)C1]

Enantiomer 1 (Example 430a, (1R,3S)-3-(3-((5-fluoro-2,2-dioxido-3,4-dihydro-1H-benzo[c][1,2]thiazin-6-yl)amino)-1H-pyrazol-5-yl)cyclopentyl isopropylcarbamate, 100% ee); Retention time: 1.659 min. ¹H NMR (500 MHz, DMSO-d₆) δ 11.69 (s, 1H), 9.77 (s, 1H), 7.84-7.81 (m, 2H), 6.94 (d, J=7.3, 1H), 6.50 (d, J=8.9, 1H), 5.68 (s, 1H), 4.99 (s, 1H), 3.61-3.55 (m, 1H), 3.36-3.23 (m, 4H), 3.06-2.98 (m, 1H), 2.48-2.41 (m, 1H), 2.03-1.55 (m, 5H), 1.03-1.02 (m, 6H). LC-MS (ESI): m/z [M+H]⁺=452.3.

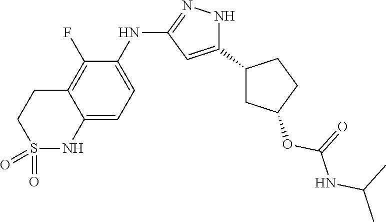 [SMILES: CC(C)NC(=O)O[C@H]1CC[C@@H](c2cc(Nc3ccc4c(c3F)CCS(=O)(=O)N4)n[nH]2)C1]

Enantiomer 2 (Example 430b, (1S,3R)-3-(3-((5-fluoro-2,2-dioxido-3,4-dihydro-1H-benzo[c][1,2]thiazin-6-yl)amino)-1H-pyrazol-5-yl)cyclopentyl isopropylcarbamate, 100% ee); Retention time: 1.664 min. ¹H NMR (500 MHz, DMSO-d₆) δ 11.67 (s, 1H), 9.76 (s, 1H), 7.88-7.81 (m, 2H), 6.94 (d, J=7.3, 1H), 6.50 (d, J=8.9, 1H), 5.67 (s, 1H), 4.99 (s, 1H), 3.60-3.55 (m, 1H), 3.37-3.25 (m, 4H), 3.03-2.98 (m, 1H), 2.48-2.41 (m, 1H), 2.02-1.55 (m, 5H), 1.03-1.02 (m, 6H). LC-MS (ESI): m/z [M+H]⁺=452.3.

Chiral analytical method: Column: CHIRALPAK IE 4.6 mm×250 mm, 5 μm; Mobile phase: A for MTBE and B for DCM:MeOH=1:1 (0.1% 2M NH₃ MeOH); Gradient: Mobile Phase A: Mobile Phase B=50: 50 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MTBE and B for DCM:MeOH=1:1 (0.2% 2M NH₃ MeOH); Gradient: Mobile Phase A: Mobile Phase B=50: 50 (v/v); Flow Rate: 18 mL/min, Wavelength: UV 200 nm and 270 nm, Prep-HPLC Equipment: Prep-HPLC-Gilson, Back pressure: 100 bar; Column temperature: 25° C.

### Example 431: cis-3-(3-((4-fluoro-2,2-dioxido-1,3-dihydrobenzo[c]isothiazol-5-yl)amino)-1H-pyrazol-5-yl)cyclopentyl(tetrahydrofuran-3-yl)carbamate

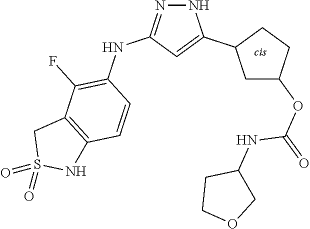 [SMILES: O=C(NC1CCOC1)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.65 (s, 1H), 8.19 (s, OH), 7.86 (s, 1H), 7.78 (s, 1H), 7.34 (d, J=5.5 Hz, 1H), 6.55 (d, J=8.6 Hz, 1H), 5.65 (s, 1H), 5.00 (s, 1H), 4.53 (s, 2H), 4.02 (s, 1H), 3.79-3.68 (m, 2H), 3.67-3.60 (m, 1H), 3.45-3.41 (m, 1H), 3.08-2.98 (m, 1H), 2.44-2.35 (m, 1H), 2.08-1.96 (m, 2H), 1.94-1.84 (m, 1H), 1.80-1.66 (m, 3H), 1.64-1.54 (m, 1H). LC-MS (ESI): m/z [M+H]⁺=466.2.

### Example 432: cis-3-(3-((4-fluoro-2,2-dioxido-1,3-dihydrobenzo[c]isothiazol-5-yl)amino)-1H-pyrazol-5-yl)cyclopentyl(2-oxobutyl)carbamate

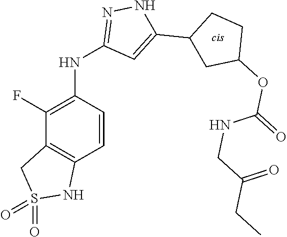 [SMILES: CCC(=O)CNC(=O)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.67 (s, 1H), 10.26 (s, 1H), 7.90 (s, 1H), 7.81 (s, 1H), 7.27 (t, J=5.8 Hz, 1H), 6.55 (d, J=12.8 Hz, 1H), 5.65 (s, 1H), 4.99 (s, 1H), 4.55 (s, 2H), 3.80 (d, J=5.8 Hz, 2H), 3.11-2.95 (m, 1H), 2.48-2.28 (m, 3H), 2.06-1.97 (m, 1H), 1.97-1.85 (m, 1H), 1.81-1.66 (m, 2H), 1.66-1.55 (m, 1H), 0.92 (t, J=7.3 Hz, 3H). LC-MS (ESI): m/z [M+H]⁺=466.2.

### Example 433: cis-3-(3-((4-fluoro-1-(2-hydroxy-2-methylpropyl)-2,2-dioxido-1,3-dihydrobenzo[c]isothiazol-5-yl)amino)-1H-pyrazol-5-yl)cyclopentyl propylcarbamate

### Step 1:5-bromo-4-fluoro-1-(2-hydroxy-2-methylpropyl)-1,3-dihydrobenzo[c]isothiazole 2,2-dioxide

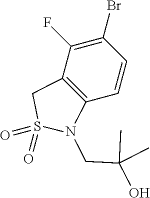 [SMILES: CC(C)(O)CN1c2ccc(Br)c(F)c2CS1(=O)=O]

A mixture of 2-methylpropane-1,2-diol (450 mg, 5.01 mmol), CMBP (2.7 g, 11.2 mmol) and 5-bromo-4-fluoro-2,3-dihydrobenzo[d]isothiazole 1,1-dioxide (1 g, 3.76 mmol) in toluene (20 mL) was stirred at 95° C. under N₂ for 12 hr. The mixture was concentrated under reduced pressure. The residue was purified by silica gel column chromatography, eluting with PE/EtOAc (2: 1~1:1) to afford the product (320 mg, 25.2%). LC-MS (ESI): m/z [M+H]⁺=338.0.

### Step 2: cis-3-(3-((4-fluoro-1-(2-hydroxy-2-methylpropyl)-2,2-dioxido-1,3-dihydrobenzo[c]isothiazol-5-yl)amino)-1H-pyrazol-5-yl)cyclopentyl propylcarbamate

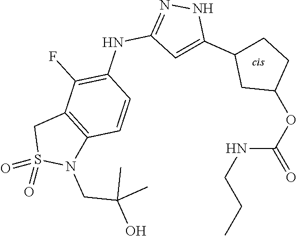 [SMILES: CCCNC(=O)OC1CCC(c2cc(Nc3ccc4c(c3F)CS(=O)(=O)N4CC(C)(C)O)n[nH]2)C1]

A mixture of 5-bromo-4-fluoro-1-(2-hydroxy-2-methylpropyl)-1,3-dihydrobenzo[c]isothiazole 2,2-dioxide (150 mg, 0.44 mmol), 3-(3-amino-1H-pyrazol-5-yl)cyclopentyl propylcarbamate (111.8 mg, 0.44 mmol), Brettphos G3 Pd (39.8 mg, 0.044 mmol), K₂CO₃ (182.2 mg, 1.32 mmol) in t-BuOH (20 mL) was stirred at 110° C. for 16 h under N₂ atmosphere. The mixture was concentrated under reduced pressure. The residue was purified by silica gel column chromatography, eluting with EA to afford the product in a racemic form, which was further separated by Chiral Prep-HPLC to give:

Enantiomer 1 (Example 433a, 100% ee); Retention time: 9.71 min. ¹H NMR (500 MHz, DMSO-d₆) δ 11.69 (s, 1H), 7.97 (t, J=8.7 Hz, 1H), 7.88 (s, 1H), 7.04 (t, J=5.4 Hz, 1H), 6.91 (d, J=8.9 Hz, 1H), 5.67 (s, 1H), 4.99 (s, 1H), 4.70 (s, 2H), 4.66 (s, 1H), 3.08-2.98 (m, 1H), 2.95-2.93 (m, 2H), 2.48-2.39 (m, 1H), 2.06-1.97 (m, 1H), 1.95-1.85 (m, 1H), 1.79-1.64 (m, 2H), 1.62-1.54 (m, 1H), 1.47-1.29 (m, 2H), 1.19 (s, 6H), 0.82 (t, J=7.4 Hz, 3H). LC-MS (ESI): m/z [M+H]⁺=510.2.

Enantiomer 2 (Example 433b, 100% ee); Retention time: 11.62 min. ¹H NMR (500 MHz, DMSO-d₆) δ 11.69 (s, 1H), 7.97 (t, J=8.7 Hz, 1H), 7.88 (s, 1H), 7.04 (t, J=5.4 Hz, 1H), 6.91 (d, J=8.9 Hz, 1H), 5.67 (s, 1H), 4.99 (s, 1H), 4.70 (s, 2H), 4.66 (s, 1H), 3.08-2.98 (m, 1H), 2.95-2.93 (m, 2H), 2.48-2.39 (m, 1H), 2.06-1.97 (m, 1H), 1.95-1.85 (m, 1H), 1.79-1.64 (m, 2H), 1.62-1.54 (m, 1H), 1.47-1.29 (m, 2H), 1.19 (s, 6H), 0.82 (t, J=7.4 Hz, 3H). LC-MS (ESI): m/z [M+H]⁺=510.2.

Chiral analytical method: Column: CHIRALPAK IE 4.6 mm×250 mm, 5 μm; Mobile phase: A for MtBE (0.1% 2M NH₃ MeOH) and B for DCM:MeOH=50: 50 (v/v); Gradient: Mobile Phase A: Mobile Phase B=80: 20 (v/v); HPLC Equipment: HPLC-Agilent, Back pressure: 100 bar; Column temperature: 35° C.

Chiral Prep-HPLC Condition: CHIRALPAK IE 21.2 mm×250 mm, 5 μm; Mobile phase: A for MtBE and B for DCM:MeOH=50: 50 (v/v) (0.2% 2M NH₃ MeOH); Gradient: Mobile Phase A: Mobile Phase B=80: 20 (v/v); Flow Rate: 18 mL/min, Wave Length: UV 270 nm and 320 nm, Prep-HPLC Equipment: Prep-HPLC-Gilson, Back pressure: 100 bar; Column temperature: 25° C.

### Example 434: cis-3-(3-((4-fluoro-1-(2-hydroxy-2-methylpropyl)-2,2-dioxido-1,3-dihydrobenzo[c]isothiazol-5-yl)amino)-1H-pyrazol-5-yl)cyclopentyl((S)-sec-butyl)carbamate

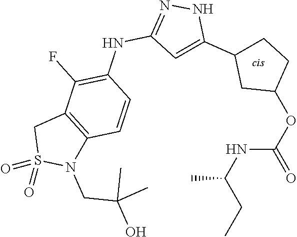 [SMILES: CC[C@H](C)NC(=O)OC1CCC(c2cc(Nc3ccc4c(c3F)CS(=O)(=O)N4CC(C)(C)O)n[nH]2)C1]

The titled compound was synthesized in the procedures similar to Example 63. ¹H NMR (500 MHz, DMSO-d₆) δ=11.70 (s, 1H), 7.94 (s, 1H), 7.88 (s, 1H), 6.91 (d, J=8.9, 1H), 6.88 (d, J=8.2, 1H), 5.67 (s, 1H), 4.98 (m, 1H), 4.71 (s, 2H), 4.66 (s, 1H), 3.38 (m, 1H), 3.31-3.26 (m, 2H), 3.02 (m, 1H), 2.44 (m, 1H), 1.99 (m, 1H), 1.91 (m, 1H), 1.70 (m, 2H), 1.58 (m, 1H), 1.35 (m, 2H), 1.20 (s, 6H), 1.00 (m, 3H), 0.80 (m, 3H). LC-MS (ESI): m/z [M+H]⁺=524.36.

### Example 435: cis-3-(3-((4-fluoro-1-(2-hydroxy-2-methylpropyl)-2,2-dioxido-1,3-dihydrobenzo[c]isothiazol-5-yl)amino)-1H-pyrazol-5-yl)cyclopentyl(1-methylcyclopropyl)carbamate

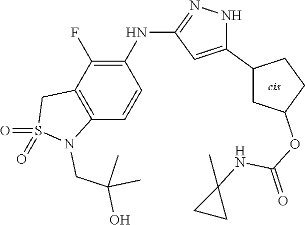 [SMILES: CC(C)(O)CN1c2ccc(Nc3cc(C4CCC(OC(=O)NC5(C)CC5)C4)[nH]n3)c(F)c2CS1(=O)=O]

The titled compound was synthesized in the procedures similar to Example 63. ¹H NMR (500 MHz, DMSO-d₆) δ=11.72 (s, 1H), 7.94 (s, 1H), 7.88 (s, 1H), 7.34 (s, 1H), 6.91 (d, J=8.8, 1H), 5.66 (s, 1H), 4.98 (m, 1H), 4.70 (s, 2H), 4.66 (s, 1H), 3.32-3.28 (m, 2H), 3.01 (m, 1H), 2.45 (m, 1H), 1.99 (m, 1H), 1.90 (m, 1H), 1.68 (m, 2H), 1.55 (m, 1H), 1.23 (s, 3H), 1.20 (s, 6H), 0.59 (m, 2H), 0.47 (m, 2H).

LC-MS (ESI): m/z [M+H]⁺=522.32.

### Example 436: cis-3-(3-((4-fluoro-2,2-dioxido-1,3-dihydrobenzo[c]isothiazol-5-yl)amino)-1H-pyrazol-5-yl)cyclopentyl 2,2-dimethylpyrrolidine-1-carboxylate

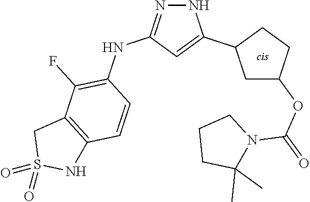 [SMILES: CC1(C)CCCN1C(=O)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.75 (s, 1H), 10.20 (s, 1H), 7.98-7.77 (m, 2H), 6.58 (d, J=8.6 Hz, 1H), 5.66 (s, 1H), 5.02 (d, J=32.9 Hz, 1H), 4.58 (s, 2H), 3.15-2.90 (m, 1H), 2.45-2.35 (m, 1H), 2.08-1.96 (m, 1H), 1.95-1.87 (m, 1H), 1.82-1.61 (m, 7H), 1.35-1.25 (m, 6H). LC-MS (ESI): m/z [M+H]⁺=478.2.

### Example 437: cis-3-(3-((4-fluoro-1,1-dioxido-2,3-dihydrobenzo[d]isothiazol-5-yl)amino)-1H-pyrazol-5-yl)cyclopentyl methyl(tetrahydrofuran-3-yl)carbamate

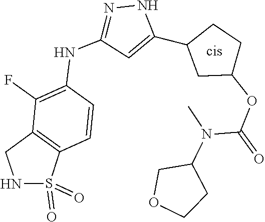 [SMILES: CN(C(=O)OC1CCC(c2cc(Nc3ccc4c(c3F)CNS4(=O)=O)n[nH]2)C1)C1CCOC1]

The titled compound was synthesized in the procedures similar to Example 328. ¹H NMR (500 MHz, DMSO-d₆) δ=12.01 (s, 1H), 8.72 (s, 1H), 8.27 (t, J=7.7, 1H), 7.69 (m, 1H), 7.48 (d, J=8.6, 1H), 5.83 (s, 1H), 5.04 (m, 1H), 4.70 (brs, 1H), 4.40 (d, J=4.5, 2H), 3.84 (m, 1H), 3.60 (d, J=5.0, 2H), 3.53 (m, 1H), 3.19-3.05 (m, 1H), 2.71 (s, 3H), 2.44 (m, 1H), 2.14-1.99 (m, 2H), 1.97-1.86 (m, 1H), 1.78 (m, 4H). LC-MS (ESI): m/z [M+H]⁺=480.30.

### Example 438: cis-3-(3-((4-fluoro-2,2-dioxido-1,3-dihydrobenzo[c]isothiazol-5-yl)amino)-1H-pyrazol-5-yl)cyclopentyl(3,3,3-trifluoropropyl)carbamate

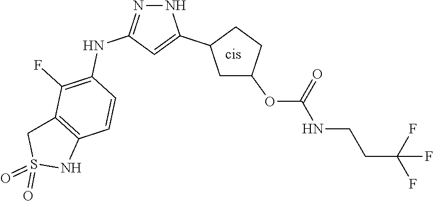 [SMILES: O=C(NCCC(F)(F)F)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.69 (s, 1H), 10.20 (s, 1H), 7.89 (d, J=40.1 Hz, 2H), 7.26 (t, J=5.7 Hz, 1H), 6.59 (d, J=8.7 Hz, 1H), 5.66 (s, 1H), 5.01 (s, 1H), 4.58 (s, 2H), 3.21 (dd, J=12.9, 6.7 Hz, 2H), 3.07-2.98 (m, 1H), 2.48-2.35 (m, 3H), 2.04-1.87 (m, 2H), 1.75-1.56 (m, 3H). LC-MS (ESI): m/z [M+H]⁺=492.3.

### Example 439: cis-3-(3-((4-fluoro-2,2-dioxido-1,3-dihydrobenzo[c]isothiazol-5-yl)amino)-1H-pyrazol-5-yl)cyclopentyl(1-methylcyclopentyl)carbamate

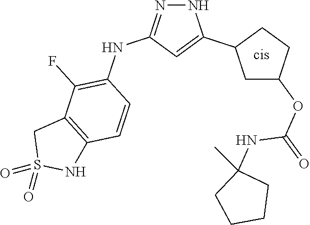 [SMILES: CC1(NC(=O)OC2CCC(c3cc(Nc4ccc5c(c4F)CS(=O)(=O)N5)n[nH]3)C2)CCCC1]

The titled compound was synthesized in the procedures similar to Example 251. ¹H NMR (500 MHz, DMSO-d₆) δ 11.61 (s, 1H), 8.26 (s, 1H), 7.58 (s, 1H), 6.84 (s, 1H), 6.55-6.41 (m, 2H), 5.60 (s, 1H), 4.95 (s, 1H), 4.42-4.35 (m, 2H), 3.05-2.95 (m, 1H), 2.43-2.40 (m, 1H), 2.03-1.84 (m, 5H), 1.75-1.65 (m, 2H), 1.64-1.48 (m, 4H), 1.45-1.35 (m, 2H), 1.26 (s, 3H). LC-MS (ESI): m/z [M+H]⁺=478.3.

### Example 440: cis-3-(3-((6-fluoro-1,1-dioxido-2,3-dihydrobenzo[d]isothiazol-5-yl)amino)-1H-pyrazol-5-yl)cyclopentyl methyl(tetrahydrofuran-3-yl)carbamate

### Step 1: cis-3-(3-amino-1H-pyrazol-5-yl)cyclopentyl methyl(tetrahydrofuran-3-yl)carbamate

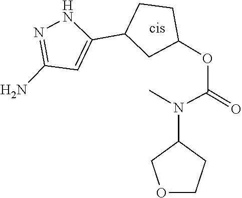 [SMILES: CN(C(=O)OC1CCC(c2cc(N)n[nH]2)C1)C1CCOC1]

The titled compound was synthesized in the procedures similar to Example 34. LC-MS (ESI): m/z [M+H]⁺=295.2.

### Step 2: cis-3-(3-((6-fluoro-1,1-dioxido-2,3-dihydrobenzo[d]isothiazol-5-yl)amino)-1H-pyrazol-5-yl)cyclopentyl methyl(tetrahydrofuran-3-yl)carbamate

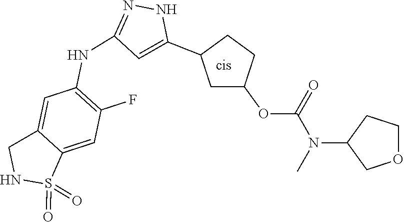 [SMILES: CN(C(=O)OC1CCC(c2cc(Nc3cc4c(cc3F)S(=O)(=O)NC4)n[nH]2)C1)C1CCOC1]

The titled compound was synthesized in the procedures similar to Example 34. ¹H NMR (500 MHz, DMSO-d₆) δ 12.00 (s, 1H), 8.75 (d, J=2.2 Hz, 1H), 8.16 (d, J=7.3 Hz, 1H), 7.65 (s, 2H), 5.85 (s, 1H), 5.04 (d, J=2.5 Hz, 1H), 4.71 (s, 1H), 4.30 (s, 2H), 3.85 (d, J=5.0 Hz, 1H), 3.60 (d, J=4.9 Hz, 2H), 3.53 (d, J=5.9 Hz, 1H), 3.11 (dd, J=16.2, 8.1 Hz, 1H), 2.71 (s, 3H), 2.47-2.39 (m, 1H), 2.13-2.00 (m, 2H), 1.91 (tt, J=16.5, 6.4 Hz, 1H), 1.83-1.68 (m, 4H). LC-MS (ESI): m/z [M+H]⁺=480.2.

### Example 441: cis-3-(3-((4-fluoro-2,2-dioxido-1,3-dihydrobenzo[c]isothiazol-5-yl)amino)-1H-pyrazol-5-yl)cyclopentyl methyl(tetrahydrofuran-3-yl)carbamate

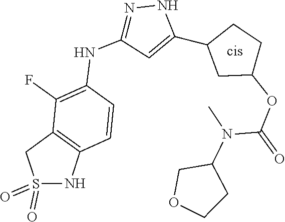 [SMILES: CN(C(=O)OC1CCC(c2cc(Nc3ccc4c(c3F)CS(=O)(=O)N4)n[nH]2)C1)C1CCOC1]

The titled compound was synthesized in the procedures similar to Example 34. ¹H NMR (500 MHz, DMSO-d₆) δ 11.71 (s, 1H), 10.21 (s, 1H), 7.91 (s, 1H), 7.86 (s, 1H), 6.59 (d, J=8.6 Hz, 1H), 5.67 (s, 1H), 5.03 (s, 1H), 4.68 (d, J=48.3 Hz, 1H), 4.58 (s, 2H), 3.85 (d, J=5.1 Hz, 1H), 3.60 (d, J=5.0 Hz, 2H), 3.53 (d, J=6.9 Hz, 1H), 3.06 (dd, J=16.1, 8.1 Hz, 1H), 2.71 (s, 3H), 2.45-2.34 (m, 1H), 2.10-1.96 (m, 2H), 1.95-1.85 (m, 1H), 1.83-1.65 (m, 4H). LC-MS (ESI): m/z [M+H]⁺=480.2.

### Example 442: cis-3-(3-((4-fluoro-1,1-dioxido-2,3-dihydrobenzo[d]isothiazol-5-yl)amino)-1H-pyrazol-5-yl)cyclopentyl((1R,2R)-2-hydroxycyclopropyl)carbamate

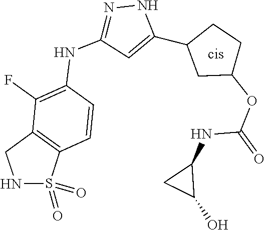 [SMILES: O=C(N[C@@H]1C[C@H]1O)OC1CCC(c2cc(Nc3ccc4c(c3F)CNS4(=O)=O)n[nH]2)C1]

The titled compound was synthesized in the procedures similar to Example 328. ¹H NMR (500 MHz, DMSO-d₆) δ=11.98 (s, 1H), 8.71 (s, 1H), 8.23 (s, 1H), 7.69 (t, J=4.9, 1H), 7.48 (d, J=8.6, 1H), 7.11 (s, 1H), 5.91-5.78 (m, 1H), 5.38 (s, 1H), 5.00 (m, 1H), 4.41 (d, J=4.8, 2H), 3.13 (m, 1H), 3.10-3.02 (m, 1H), 2.48-2.42 (m, 1H), 2.41-2.36 (m, 1H), 2.05-1.98 (m, 1H), 1.91 (m, 1H), 1.70 (m, 2H), 1.59 (m, 1H), 0.74-0.66 (m, 1H), 0.57 (m, 1H). LC-MS (ESI): m/z [M+H]⁺=452.26.

### Example 443: cis-3-(3-((4-fluoro-2,2-dioxido-1,3-dihydrobenzo[c]isothiazol-5-yl)amino)-1H-pyrazol-5-yl)cyclopentyl(1-hydroxy-2-methylpropan-2-yl)carbamate

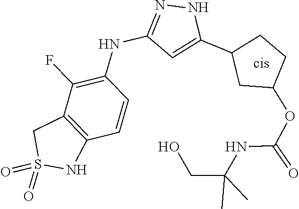 [SMILES: CC(C)(CO)NC(=O)OC1CCC(c2cc(Nc3ccc4c(c3F)CS(=O)(=O)N4)n[nH]2)C1]

The titled compound was synthesized in the procedures similar to example 251. ¹H NMR (500 MHz, DMSO-d₆) δ 11.68 (s, 1H), 10.18 (s, 1H), 7.92 (s, 1H), 7.84 (s, 1H), 6.58 (d, J=8.6 Hz, 1H), 6.47 (s, 1H), 5.67 (s, 1H), 5.08-4.90 (m, 1H), 4.70 (t, J=5.8 Hz, 1H), 4.58 (s, 2H), 3.29 (s, 2H), 3.04-2.97 (m, 1H), 2.47-2.39 (m, 1H), 2.05-1.95 (m, 1H), 1.94-1.82 (m, 1H), 1.77-1.64 (m, 2H), 1.62-1.50 (m, 1H), 1.14 (s, 6H) LC-MS (ESI): m/z [M+H]⁺=468.30.

### Example 444: cis-3-(3-((6-fluoro-1,1-dioxido-2,3-dihydrobenzo[d]isothiazol-5-yl)amino)-1H-pyrazol-5-yl)cyclopentyl(1-hydroxy-2-methylpropan-2-yl)carbamate

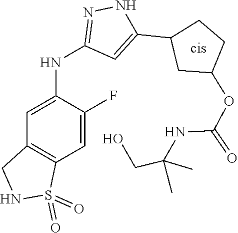 [SMILES: CC(C)(CO)NC(=O)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 12.01 (brs, 1H), 8.74 (d, J=2.2 Hz, 1H), 8.13 (d, J=7.1 Hz, 1H), 7.64 (d, J=10.1 Hz, 2H), 6.47 (s, 1H), 5.85 (s, 1H), 5.05-4.92 (m, 1H), 4.30 (s, 2H), 3.32 (s, 2H), 3.14-2.97 (m, 1H), 2.49-2.43 (m, 1H), 2.06-1.97 (m, 1H), 1.95-1.84 (m, 1H), 1.79-1.66 (m, 2H), 1.65-1.52 (m, 1H), 1.14 (s, 6H). LC-MS (ESI): m/z [M+H]⁺=468.29.

### Example 445: cis-3-(3-((6-fluoro-1,1-dioxido-2,3-dihydrobenzo[d]isothiazol-5-yl)amino)-1H-pyrazol-5-yl)cyclopentyl(1-methylcyclobutyl)carbamate

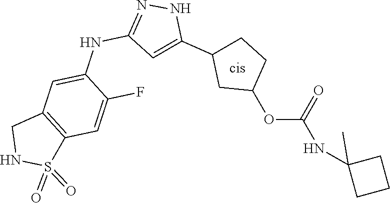 [SMILES: CC1(NC(=O)OC2CCC(c3cc(Nc4cc5c(cc4F)S(=O)(=O)NC5)n[nH]3)C2)CCC1]

The titled compound was synthesized in the procedures similar to Example 412. ¹H NMR (500 MHz, DMSO-d₆) δ 11.97 (s, 1H), 8.74 (s, 1H), 8.15 (d, J=7.0 Hz, 1H), 7.71-7.57 (m, 2H), 7.16 (s, 1H), 5.85 (s, 1H), 4.99 (s, 1H), 4.30 (d, J=4.8 Hz, 2H), 3.11-3.00 (m, 1H), 2.46-2.43 (m, 1H), 2.30-2.15 (m, 2H), 2.08-1.84 (m, 2H), 1.84-1.54 (m, 7H), 1.31 (s, 3H). LC-MS (ESI): m/z [M+H]⁺=464.3.

### Example 446: cis-3-(3-((6-fluoro-1,1-dioxido-2,3-dihydrobenzo[d]isothiazol-5-yl)amino)-1H-pyrazol-5-yl)cyclopentyl(1-methylcyclopentyl)carbamate

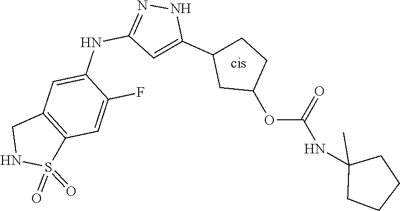 [SMILES: CC1(NC(=O)OC2CCC(c3cc(Nc4cc5c(cc4F)S(=O)(=O)NC5)n[nH]3)C2)CCCC1]

The titled compound was synthesized in the procedures similar to Example 412. ¹H NMR (500 MHz, DMSO-d₆) δ 11.97 (s, 1H), 8.74 (s, 1H), 8.13 (s, 1H), 7.71-7.59 (m, 2H), 6.84 (s, 1H), 5.85 (s, 1H), 4.98 (s, 1H), 4.30 (d, J=5.0 Hz, 2H), 3.10-3.02 (m, 1H), 2.49-2.42 (m, 1H), 2.05-1.85 (m, 4H), 1.78-1.66 (m, 2H), 1.64-1.51 (m, 5H), 1.48-1.39 (m, 2H), 1.26 (s, 3H). LC-MS (ESI): m/z [M+H]⁺=478.3.

### Example 447: cis-3-(3-((6-fluoro-1,1-dioxido-2,3-dihydrobenzo[d]isothiazol-5-yl)amino)-1H-pyrazol-5-yl)cyclopentyl 2,2-dimethylpyrrolidine-1-carboxylate

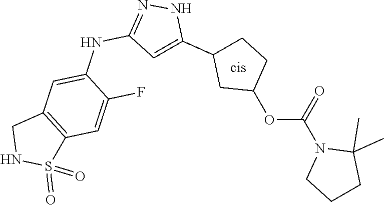 [SMILES: CC1(C)CCCN1C(=O)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 11.99 (s, 1H), 8.75 (s, 1H), 8.15 (s, 1H), 7.68-7.60 (m, 2H), 5.84 (s, 1H), 5.03 (d, J=34.3 Hz, 1H), 4.30 (d, J=4.7 Hz, 2H), 3.13-3.04 (m, 1H), 2.48-2.35 (m, 3H), 2.09-1.98 (m, 1H), 1.93-1.85 (m, 1H), 1.81-1.62 (m, 7H), 1.33-1.24 (m, 6H). LC-MS (ESI): m/z [M+H]⁺=478.3.

### Example 448: cis-3-(3-((4-fluoro-2,2-dioxido-1,3-dihydrobenzo[c]isothiazol-5-yl)amino)-1H-pyrazol-5-yl)cyclopentyl bicyclo[1.1.1]pentan-1-ylcarbamate

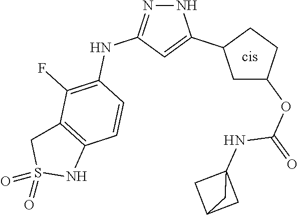 [SMILES: O=C(NC12CC(C1)C2)OC1CCC(c2cc(Nc3ccc4c(c3F)CS(=O)(=O)N4)n[nH]2)C1]

The titled compound was synthesized in the procedures similar to Example 251. ¹H NMR (500 MHz, DMSO-d₆) δ 11.68 (s, 1H), 10.20 (s, 1H), 7.94 (s, 1H), 7.85 (s, 1H), 7.75 (s, 1H), 6.58 (d, J=8.7 Hz, 1H), 5.66 (s, 1H), 4.98 (s, 1H), 4.58 (s, 2H), 3.07-2.98 (m, 1H), 2.48-2.42 (m, 1H), 2.05-1.96 (m, 1H), 1.94-1.83 (m, 7H), 1.78-1.53 (m, 3H). LC-MS (ESI): m/z [M+H]⁺=462.3.

### Example 449: cis-3-(3-((6-fluoro-1,1-dioxido-2,3-dihydrobenzo[d]isothiazol-5-yl)amino)-1H-pyrazol-5-yl)cyclopentyl 2,2-dimethylazetidine-1-carboxylate

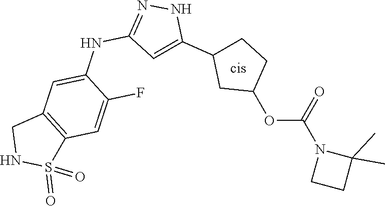 [SMILES: CC1(C)CCN1C(=O)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 12.01 (s, 1H), 8.75 (s, 1H), 8.15 (d, J=6.4 Hz, 1H), 7.71-7.59 (m, 2H), 5.84 (s, 1H), 5.01 (d, J=20.1 Hz, 1H), 4.30 (d, J=4.9 Hz, 2H), 3.69 (dt, J=38.7, 7.6 Hz, 2H), 3.18-3.05 (m, 1H), 2.48-2.41 (m, 1H), 2.09-1.83 (m, 4H), 1.79-1.60 (m, 3H), 1.41-1.29 (m, 6H). LC-MS (ESI): m/z [M+H]⁺=464.3.

### Example 450: cis-3-(3-((6-fluoro-1,1-dioxido-2,3-dihydrobenzo[d]isothiazol-5-yl)amino)-1H-pyrazol-5-yl)cyclopentyl(3-methyltetrahydrofuran-3-yl)carbamate

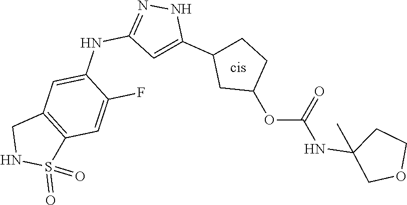 [SMILES: CC1(NC(=O)OC2CCC(c3cc(Nc4cc5c(cc4F)S(=O)(=O)NC5)n[nH]3)C2)CCOC1]

The titled compound was synthesized in the procedures similar to Example 412. ¹H NMR (500 MHz, DMSO-d₆) δ=11.99 (s, 1H), 8.74 (s, 1H), 8.15 (s, 1H), 7.77-7.52 (m, 2H), 7.23 (s, 1H), 5.85 (s, 1H), 5.00 (m, 1H), 4.30 (d, J=4.4, 2H), 3.82 (d, J=7.9, 1H), 3.73 (t, J=7.1, 2H), 3.44 (d, J=8.6, 1H), 3.11-3.02 (m, 1H), 2.49-2.44 (m, 1H), 2.16 (m, 1H), 2.06-1.99 (m, 1H), 1.97-1.84 (m, 1H), 1.79-1.65 (m, 3H), 1.60 (m, 1H), 1.32 (s, 3H). LC-MS (ESI): m/z [M+H]⁺=480.30.

### Example 451: cis-3-(3-((6-fluoro-1,1-dioxido-2,3-dihydrobenzo[d]isothiazol-5-yl)amino)-1H-pyrazol-5-yl)cyclopentyl(3-methyloxetan-3-yl)carbamate

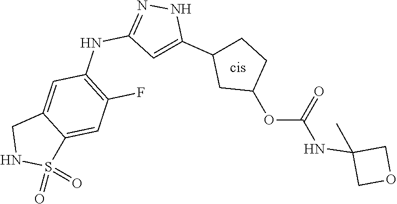 [SMILES: CC1(NC(=O)OC2CCC(c3cc(Nc4cc5c(cc4F)S(=O)(=O)NC5)n[nH]3)C2)COC1]

The titled compound was synthesized in the procedures similar to Example 412. ¹H NMR (500 MHz, DMSO-d₆) δ=12.02 (s, 1H), 8.75 (s, 1H), 8.13 (m, 1H), 7.72-7.53 (m, 3H), 5.86 (s, 1H), 5.02 (m, 1H), 4.56 (s, 2H), 4.30 (d, J=4.8, 2H), 4.24 (d, J=4.2, 2H), 3.12-3.02 (m, 1H), 2.47 (m, 1H), 2.03 (m, 1H), 1.92 (m, 1H), 1.74 (m, 2H), 1.62 (m, 1H), 1.47 (s, 3H). LC-MS (ESI): m/z [M+H]⁺=466.28.

### Example 452: cis-3-(3-((4-fluoro-1-methyl-2,2-dioxido-1,3-dihydrobenzo[c]isothiazol-5-yl)amino)-1H-pyrazol-5-yl)cyclopentyl bicyclo[1.1.1]pentan-1-ylcarbamate

### Step 1: cis-3-(3-(((benzyloxy) carbonyl)amino)-1H-pyrazol-5-yl)cyclopentyl bicyclo[1.1.1]pentan-1-ylcarbamate

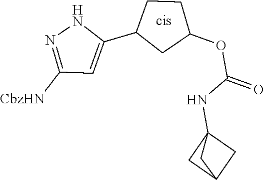 [SMILES: O=C(Nc1cc(C2CCC(OC(=O)NC34CC(C3)C4)C2)[nH]n1)OCc1ccccc1]

To a solution of cis-benzyl(5-(3-(((4-nitrophenoxy) carbonyl)oxy) cyclopentyl)-1H-pyrazol-3-yl)carbamate (7 g, 15 mmol) in 100 mL THF was added bicyclo[1.1.1]pentan-3-amine hydrochloride (2.5 g, 30 mmol) and DIEA (7.7 g, 60 mmol). The resulting mixture was stirred at room temperature for 1 h. The resulting mixture was concentrated under reduced pressure, and the residue was purified by silica gel chromatography (PE/EA=2:1) to afford the product (4.6 g, 75% yield). LC-MS (ESI): m/z [M+H]⁺=411.3.

### Step 2: cis-3-(3-amino-1H-pyrazol-5-yl)cyclopentyl bicyclo[1.1.1]pentan-1-ylcarbamate

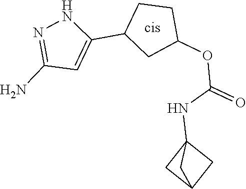 [SMILES: Nc1cc(C2CCC(OC(=O)NC34CC(C3)C4)C2)[nH]n1]

To a solution of cis-3-(3-(((benzyloxy) carbonyl)amino)-1H-pyrazol-5-yl)cyclopentyl bicyclo[1.1.1]pentan-1-ylcarbamate (4.6 g, 13.5 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (3.2 g, 85% yield). LC-MS (ESI): m/z [M+H]⁺=277.2.

### Step 3: cis-3-(3-((4-fluoro-1-methyl-2,2-dioxido-1,3-dihydrobenzo[c]isothiazol-5-yl)amino)-1H-pyrazol-5-yl)cyclopentyl bicyclo[1.1.1]pentan-1-ylcarbamate

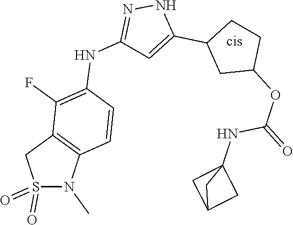 [SMILES: CN1c2ccc(Nc3cc(C4CCC(OC(=O)NC56CC(C5)C6)C4)[nH]n3)c(F)c2CS1(=O)=O]

The titled compound was synthesized in the procedures similar to example 203. ¹H NMR (500 MHz, DMSO-d₆) δ 11.63 (s, 1H), 8.22 (s, 1H), 7.76 (s, 1H), 7.41 (s, 1H), 7.25 (s, 1H), 6.78 (d, J=8.6 Hz, 1H), 5.57 (s, 1H), 5.10-4.90 (s, 1H), 4.56 (s, 2H), 3.09-2.96 (m, 1H), 2.47-2.40 (m, 1H), 2.38-2.30 (m, 1H), 2.06-1.96 (m, 1H), 1.94-1.85 (m, 7H), 1.78-1.53 (m, 3H). LC-MS (ESI): m/z [M+H]⁺=476.35.

### Example 453: cis-3-(3-((4-fluoro-1-methyl-2,2-dioxido-1,3-dihydrobenzo[c]isothiazol-5-yl)amino)-1H-pyrazol-5-yl)cyclopentyl tert-butylcarbamate

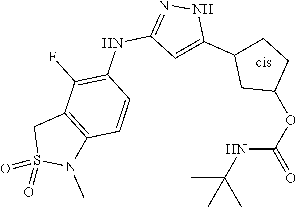 [SMILES: CN1c2ccc(Nc3cc(C4CCC(OC(=O)NC(C)(C)C)C4)[nH]n3)c(F)c2CS1(=O)=O]

The titled compound was synthesized in the procedures similar to Example 327. ¹H NMR (500 MHz, DMSO-d₆) δ 11.70 (s, 1H), 8.08-7.96 (m, 1H), 7.92 (s, 1H), 6.80-6.72 (m, 1H), 6.68 (d, J=5.0 Hz, 1H), 6.68 (s, 1H), 5.02-4.92 (m, 1H), 4.72 (s, 2H), 3.06-2.95 (m, 4H), 2.48-2.41 (m, 1H), 2.05-1.95 (m, 1H), 1.94-1.82 (m, 1H), 1.77-1.63 (m, 2H), 1.62-1.50 (m, 1H), 1.20 (m, 9H). LC-MS (ESI): m/z [M+H]⁺=466.1.

### Example 454: cis-3-(3-((1-(methyl-d3)-2,2-dioxido-1,3-dihydrobenzo[c]isothiazol-5-yl)amino)-1H-pyrazol-5-yl)cyclopentyl tert-butylcarbamate

### Step 1:5-bromo-1-(methyl-d3)-1,3-dihydrobenzo[c]isothiazole 2,2-dioxide

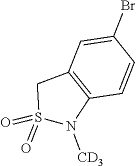 [SMILES: [2H]C([2H])([2H])N1c2ccc(Br)cc2CS1(=O)=O]

The titled compound was synthesized in the procedures similar to Example 196. LC-MS (ESI): m/z [M+Na]⁺=287.1.

### Step 2: cis-3-(3-((1-(methyl-d3)-2,2-dioxido-1,3-dihydrobenzo[c]isothiazol-5-yl)amino)-1H-pyrazol-5-yl)cyclopentyl tert-butylcarbamate

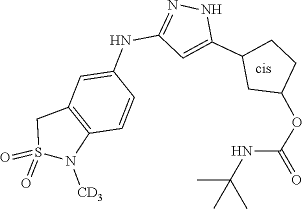 [SMILES: [2H]C([2H])([2H])N1c2ccc(Nc3cc(C4CCC(OC(=O)NC(C)(C)C)C4)[nH]n3)cc2CS1(=O)=O]

The titled compound was synthesized in the procedures similar to Example 394. ¹H NMR (500 MHz, DMSO-d₆) δ 11.73 (s, 1H), 8.22 (s, 1H), 7.40 (s, 1H), 7.25 (d, J=10.0 Hz, 1H), 6.98 (d, J=10.0 Hz, 2H), 5.57 (s, 1H), 5.01-4.93 (m, 1H), 4.56 (s, 2H), 3.07-2.96 (m, 1H), 2.48-2.40 (m, 1H), 2.05-1.95 (m, 1H), 1.93-1.84 (m, 1H), 1.77-1.65 (m, 2H), 1.62-1.53 (m, 1H), 1.21 (m, 9H). LC-MS (ESI): m/z [M+H]⁺=451.2.

### Example 455: cis-3-(3-((1-(methyl-d3)-2,2-dioxido-1,3-dihydrobenzo[c]isothiazol-5-yl)amino)-1H-pyrazol-5-yl)cyclopentyl bicyclo[1.1.1]pentan-1-ylcarbamate

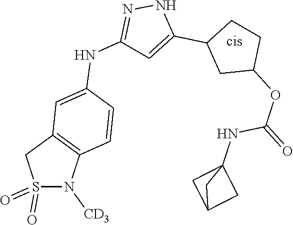 [SMILES: [2H]C([2H])([2H])N1c2ccc(Nc3cc(C4CCC(OC(=O)NC56CC(C5)C6)C4)[nH]n3)cc2CS1(=O)=O]

The titled compound was synthesized in the procedures similar to example 452. ¹H NMR (500 MHz, DMSO-d₆) δ 11.71 (s, 1H), 8.01 (s, 1H), 7.92 (s, 1H), 7.75 (s, 1H), 6.68 (d, J=8.7 Hz, 1H), 5.68 (s, 1H), 5.10-4.90 (m, 1H), 4.72 (s, 2H), 3.10-3.02 (m, 1H), 2.99 (s, 3H), 2.48-2.41 (m, 1H), 2.39-2.98 (m, 1H), 2.08-1.96 (m, 1H), 1.95-1.82 (m, 7H), 1.78-1.52 (m, 3H) . . . . LC-MS (ESI): m/z [M+H]⁺=461.31.

### Example 456: cis-3-(3-((6-fluoro-1,1-dioxido-2,3-dihydrobenzo[d]isothiazol-5-yl)amino)-1H-pyrazol-5-yl)cyclopentyl(3-hydroxybicyclo[1.1.1]pentan-1-yl)carbamate

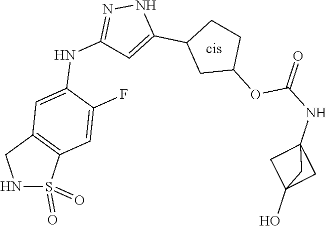 [SMILES: O=C(NC12CC(O)(C1)C2)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 11.97 (s, 1H), 8.74 (s, 1H), 8.14 (s, 1H), 7.66-7.56 (m, 2H), 6.13 (s, 1H), 5.84 (s, 1H), 4.99 (s, 1H), 4.30 (d, J=5.0 Hz, 2H), 3.11-3.01 (m, 1H), 2.47-2.40 (m, 1H), 2.06-1.97 (m, 1H), 1.93 (s, 6H), 1.89-1.82 (m, 1H), 1.79-1.64 (m, 2H), 1.62-1.54 (m, 1H). LC-MS (ESI): m/z [M+H]⁺=478.2.

### Example 457: cis-3-(3-((6-fluoro-1,1-dioxido-2,3-dihydrobenzo[d]isothiazol-5-yl)amino)-1H-pyrazol-5-yl)cyclopentyl(4-hydroxybicyclo[2.2.2]octan-1-yl)carbamate

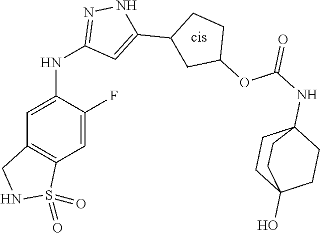 [SMILES: O=C(NC12CCC(O)(CC1)CC2)OC1CCC(c2cc(Nc3cc4c(cc3F)S(=O)(=O)NC4)n[nH]2)C1]

The titled compound was synthesized in the procedures similar to Example 412. ¹H NMR (500 MHz, DMSO-d₆) δ 11.96 (s, 1H), 8.73 (s, 1H), 8.15 (d, J=7.0 Hz, 1H), 7.72-7.49 (m, 2H), 6.65 (s, 1H), 5.83 (s, 1H), 4.94 (s, 1H), 4.30 (d, J=4.7 Hz, 2H), 4.24 (s, 1H), 3.13-2.96 (m, 1H), 2.48-2.39 (m, 1H), 2.06-1.97 (m, 1H), 1.95-1.85 (m, 1H), 1.84-1.75 (m, 6H), 1.73-1.64 (m, 2H), 1.59-1.49 (m, 7H). LC-MS (ESI): m/z [M+H]⁺=520.2.

### Example 458: cis-3-(3-((4-fluoro-1,3-dimethyl-2,2-dioxido-1,3-dihydrobenzo[c][1,2,5]thiadiazol-5-yl)amino)-1H-pyrazol-5-yl)cyclopentyl isopropylcarbamate

### Step 1:4-bromo-3-fluorobenzene-1,2-diamine

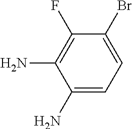

To a mixture of 4-bromo-3-fluoro-2-nitroaniline (7.3 g, 31.2 mmol) in MeOH (30 mL) were added (5.2 g, 93.6 mmol) and HCl (12N, 10 mL). The mixture was stirred at rt for 16 h. The mixture was concentrated under reduced pressure. The mixture was quenched by NaHCO₃ (100 mL), extracted with EA (3×100 mL). The combined organic layers were dried over Na₂SO₄, filtered and concentrated under vacuum. The residue was purified by silica gel column chromatography, eluting with EA/PE (0-30%) to afford (3 g, 46.9%). LC-MS (ESI): m/z [M+H]⁺=205.

### Step 2:5-bromo-4-fluoro-1,3-dihydrobenzo[c][1.2.5]thiadiazole 2,2-dioxide

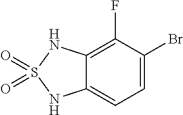 [SMILES: O=S1(=O)Nc2ccc(Br)c(F)c2N1]

To a mixture of 4-bromo-3-fluorobenzene-1,2-diamine (410 mg, 2 mmol) in pyridine (10 mL) was added Sulfamide (538 mg, 5.6 mmol). The mixture was stirred at 120° C. for 16 h. The mixture was concentrated under reduced pressure. The residue was purified by silica gel column chromatography, eluting with EA/PE (0-40%) to afford (320 mg, 59.8%). LC-MS (ESI): m/z [M+H]⁺=267.

### Step 3:5-bromo-4-fluoro-1,3-dimethyl-1,3-dihydrobenzo[c][1,2,5]thiadiazole 2,2-dioxide

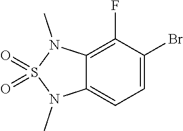

To a mixture of 5-bromo-4-fluoro-1,3-dihydrobenzo[c][1,2,5]thiadiazole 2,2-dioxide (200 mg, 0.76 mmol) and K₂CO₃ (208 mg, 1.51 mmol) in DMF was added iodomethane (214 mg, 1.51 mmol). The mixture was stirred at 50° C. for 5 h. The mixture was quenched by water (50 mL), extracted with EA (3×50 mL). The combined organic layers were dried over Na₂SO₄, filtered and concentrated under vacuum. The residue was purified by silica gel column chromatography, eluting with EA/PE (0-45%) to afford the product (134 mg, 60%). LC-MS (ESI): m/z [M+Na]⁺=317.

### Step 4: cis-3-(3-((4-fluoro-1,3-dimethyl-2,2-dioxido-1,3-dihydrobenzo[c][1.2.5]thiadiazol-5-yl)amino)-1H-pyrazol-5-yl)cyclopentyl isopropylcarbamate

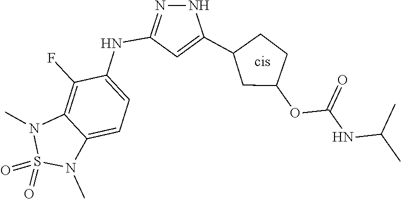 [SMILES: CC(C)NC(=O)OC1CCC(c2cc(Nc3ccc4c(c3F)N(C)S(=O)(=O)N4C)n[nH]2)C1]

A mixture of 5-bromo-4-fluoro-1,3-dimethyl-1,3-dihydrobenzo[c][1,2,5]thiadiazole 2,2-dioxide (41 mg, 0.14 mmol), cis-3-(3-amino-1H-pyrazol-5-yl)cyclopentyl isopropylcarbamate (25 mg, 0.097 mmol), Brettphos Pd G3 (25 mg, 0.097 mmol) and K₂CO₃ (58 mg, 0.42 mmol) in t-BuOH (5 mL) was stirred at 110° C. for 16 h under a nitrogen atmosphere. The mixture was concentrated under vacuum. The residue was purified by silica gel column chromatography, eluting with EA/PE (0-100%) to afford the product (11.7 mg, 26%). ¹H NMR (500 MHz, DMSO-d₆) δ 11.71 (s, 1H), 7.89 (s, 1H), 7.74-7.60 (m, 1H), 6.94 (d, J=10 Hz, 1H), 6.73 (d, J=10 Hz, 1H), 5.69 (s, 1H), 5.03-4.94 (m, 1H), 3.62-3.51 (m, 1H), 3.34 (s, 3H), 3.16 (s, 3H), 3.08-2.97 (m, 1H), 2.47-2.40 (m, 1H), 2.06-1.95 (m, 1H), 1.94-1.83 (m, 1H), 1.78-1.63 (m, 2H), 1.62-1.53 (m, 1H), 1.11-0.96 (m, 6H). LC-MS (ESI): m/z [M+H]⁺=467.2.

### Example 459: cis-3-(3-((4-fluoro-2,2-dioxido-1,3-dihydrobenzo[c]isothiazol-5-yl)amino)-1H-pyrazol-5-yl)cyclopentyl cyclopropylcarbamate

### Step 1: cis-3-(3-(((benzyloxy) carbonyl)amino)-1H-pyrazol-5-yl)cyclopentyl cyclopropylcarbamate

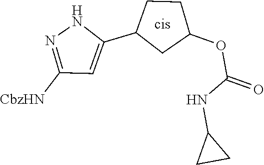 [SMILES: O=C(Nc1cc(C2CCC(OC(=O)NC3CC3)C2)[nH]n1)OCc1ccccc1]

To a solution of cis-benzyl(5-(3-(((4-nitrophenoxy) carbonyl)oxy) cyclopentyl)-1H-pyrazol-3-yl)carbamate (7 g, 15 mmol) in 100 mL THF was added cyclopropylamine (2.6 g, 45 mmol). The resulting mixture was stirred at room temperature for 1 h. The resulting mixture was concentrated under reduced pressure, and the residue was purified by silica gel chromatography (PE/EA=2:1) to afford the product (4.6 g, 80% yield). LC-MS (ESI): m/z [M+H]⁺=385.3.

### Step 2: cis-3-(3-amino-1H-pyrazol-5-yl)cyclopentyl cyclopropylcarbamate

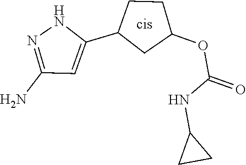 [SMILES: Nc1cc(C2CCC(OC(=O)NC3CC3)C2)[nH]n1]

To a solution of cis-3-(3-(((benzyloxy) carbonyl)amino)-1H-pyrazol-5-yl)cyclopentyl cyclopropylcarbamate (4.6 g, 12 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (2.5 g, 82% yield). LC-MS (ESI): m/z [M+H]⁺=251.4.

### Step 3: cis-3-(3-((4-fluoro-2,2-dioxido-1,3-dihydrobenzo[c]isothiazol-5-yl)amino)-1H-pyrazol-5-yl)cyclopentyl cyclopropylcarbamate

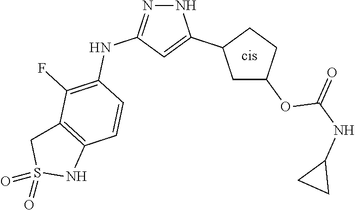 [SMILES: O=C(NC1CC1)OC1CCC(c2cc(Nc3ccc4c(c3F)CS(=O)(=O)N4)n[nH]2)C1]

The titled compound was synthesized in the procedures similar to Example 203. ¹H NMR (500 MHz, DMSO-d₆) δ 11.66 (s, 1H), 7.90 (s, 1H), 7.80 (s, 1H), 7.23 (s, 1H), 6.56 (d, J=8.7 Hz, 1H), 5.65 (s, 1H), 5.00 (s, 1H), 4.54 (s, 2H), 3.02 (s, 1H), 2.45 (dd, J=13.3, 7.4 Hz, 2H), 2.00 (d, J=9.5 Hz, 1H), 1.90 (t, J=14.7 Hz, 1H), 1.70 (s, 3H), 1.58 (s, 1H), 0.54 (d, J=5.2 Hz, 3H), 0.37 (s, 3H). LC-MS (ESI): m/z [M+H]⁺=436.1

### Example 460: cis-3-(3-((5-fluoro-2,2-dioxido-3,4-dihydro-1H-benzo[c][1,2]thiazin-6-yl)amino)-1H-pyrazol-5-yl)cyclopentyl propylcarbamate

### Step 1: cis-benzyl(1-(tert-butyl)-3-(3-(((4-nitrophenoxy) carbonyl)oxy) cyclopentyl)-1H-pyrazol-5-yl)carbamate

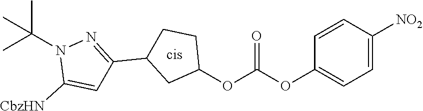 [SMILES: CC(C)(C)n1nc(C2CCC(OC(=O)Oc3ccc([N+](=O)[O-])cc3)C2)cc1NC(=O)OCc1ccccc1]

To a solution of cis-benzyl(1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 2: cis-benzyl(5-(3-(((4-nitrophenoxy) carbonyl)oxy) cyclopentyl)-1H-pyrazol-3-yl)carbamate

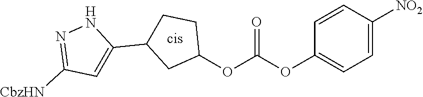 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl(1-(tert-butyl)-3-(3-(((4-nitrophenoxy) carbonyl)oxy) cyclopentyl)-1H-pyrazol-5-yl)carbamate (30 g, 57.5 mmol) was added 100 mL formic acid. The resulting mixture was stirred at 75° C. for over night. The solvent was removed under vacuum to yield 26 g of crude product, which was directly used for the next step without further purification. LC-MS (ESI): m/z [M+H]⁺=467.2.

### Step 3: cis-3-(3-(((benzyloxy) carbonyl)amino)-1H-pyrazol-5-yl)cyclopentyl propylcarbamate

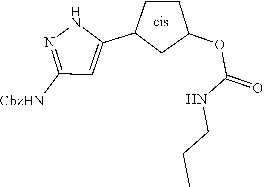 [SMILES: CCCNC(=O)OC1CCC(c2cc(NC(=O)OCc3ccccc3)n[nH]2)C1]

To a solution of cis-benzyl(5-(3-(((4-nitrophenoxy) carbonyl)oxy) cyclopentyl)-1H-pyrazol-3-yl)carbamate (7 g, 15 mmol) in 100 mL THF was added propylamine (2.6 g, 45 mmol). The resulting mixture was stirred at room temperature for 1 h. The resulting mixture was concentrated under reduced pressure, and the residue was purified by silica gel chromatography (PE/EA=2:1) to afford the product (4.6 g, 80% yield). LC-MS (ESI): m/z [M+H]⁺=387.3.

### Step 4: cis-3-(3-amino-1H-pyrazol-5-yl)cyclopentyl propylcarbamate

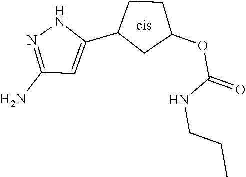 [SMILES: CCCNC(=O)OC1CCC(c2cc(N)n[nH]2)C1]

To a solution of cis-3-(3-(((benzyloxy) carbonyl)amino)-1H-pyrazol-5-yl)cyclopentyl propylcarbamate (4.6 g, 12 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with MeOH (50 mL×3). The filtrate was concentrated under reduced pressure to afford the product (2.6 g, 86% yield). LC-MS (ESI): m/z [M+H]⁺=253.4.

### Step 5: cis-3-(3-((5-fluoro-1-(4-methoxybenzyl)-2,2-dioxido-3,4-dihydro-1H-benzo[c][1.2]thiazin-6-yl)amino)-1H-pyrazol-5-yl)cyclopentyl propylcarbamate

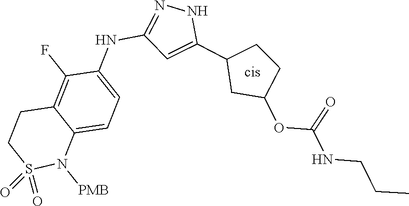

A mixture of 6-bromo-5-fluoro-1-(4-methoxybenzyl)-3,4-dihydro-1H-benzo[c][1,2]thiazine 2,2-dioxide (200 mg, 0.5 mmol), cis-3-(3-amino-1H-pyrazol-5-yl)cyclopentyl propylcarbamate (126 mg, 0.5 mmol), Brettphos Pd G3 (45.2 mg, 0.05 mmol), K₂CO₃ (207 mg, 1.5 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product (89 mg, 31% yield). LC-MS (ESI): m/z [M+H]⁺=572.2.

### Step 6: cis-3-(3-((5-fluoro-1-(4-methoxybenzyl)-2,2-dioxido-3,4-dihydro-1H-benzo [c][ 1 2]thiazin-6-yl)amino)-1H-pyrazol-5-yl)cyclopentyl propylcarbamate

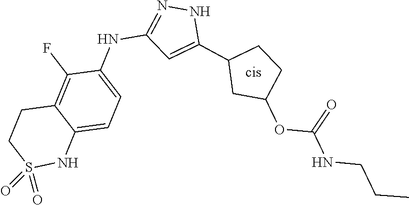 [SMILES: CCCNC(=O)OC1CCC(c2cc(Nc3ccc4c(c3F)CCS(=O)(=O)N4)n[nH]2)C1]

To a flask charged with cis-3-(3-((5-fluoro-1-methyl-2,2-dioxido-3,4-dihydro-1H-benzo[c][1,2]thiazin-6-yl)amino)-1H-pyrazol-5-yl)cyclopentyl propylcarbamate (89 mg, 0.16 mmol) was added TFA (1 mL) and DCM (3 mL). The result solution was stirred at room temperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (17 mg, 23.6% yield). ¹H NMR (500 MHz, DMSO-d₆) δ 11.68 (s, 1H), 9.76 (s, 1H), 7.87 (s, 1H), 7.80 (s, 1H), 7.04 (t, J=5.4 Hz, 1H), 6.51 (t, J=9.0 Hz, 1H), 5.68 (s, 1H), 4.99 (s, 1H), 3.25 (t, J=6.3 Hz, 4H), 3.09-2.96 (m, 1H), 2.91 (dd, J=13.3, 6.6 Hz, 2H), 2.47-2.40 (m, 1H), 2.04-1.96 (m, 1H), 1.95-1.83 (m, 1H), 1.78-1.62 (m, 2H), 1.57 (dd, J=15.8, 6.8 Hz, 1H), 1.43-1.31 (m, 2H), 0.82 (t, J=7.4 Hz, 3H). LC-MS (ESI): m/z [M+H]⁺=452.1

### Example 461: cis-3-(3-((5-fluoro-2,2-dioxido-3,4-dihydro-1H-benzo[c][1,2]thiazin-6-yl)amino)-1H-pyrazol-5-yl)cyclopentyl((S)-sec-butyl)carbamate

### Step 1: cis-benzyl(1-(tert-butyl)-3-(3-(((4-nitrophenoxy) carbonyl)oxy) cyclopentyl)-1H-pyrazol-5-yl)carbamate

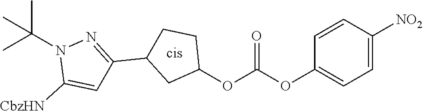 [SMILES: CC(C)(C)n1nc(C2CCC(OC(=O)Oc3ccc([N+](=O)[O-])cc3)C2)cc1NC(=O)OCc1ccccc1]

To a solution of cis-benzyl(1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 2: cis-benzyl(5-(3-(((4-nitrophenoxy) carbonyl)oxy) cyclopentyl)-1H-pyrazol-3-yl)carbamate

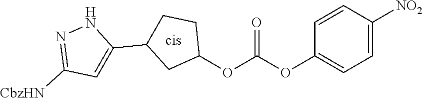 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl(1-(tert-butyl)-3-(3-(((4-nitrophenoxy) carbonyl)oxy) cyclopentyl)-1H-pyrazol-5-yl)carbamate (30 g, 57.5 mmol) was added 100 mL formic acid. The resulting mixture was stirred at 75° C. overnight. The solvent was removed under vacuum to yield 26 g of crude product, which was directly used for the next step without further purification. LC-MS (ESI): m/z [M+H]⁺=467.2.

### Step 3: cis-3-(3-(((benzyloxy) carbonyl)amino)-1H-pyrazol-5-yl)cyclopentyl((S)-sec-butyl)carbamate

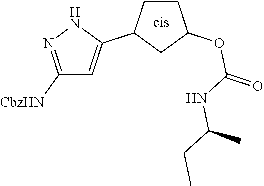 [SMILES: CC[C@H](C)NC(=O)OC1CCC(c2cc(NC(=O)OCc3ccccc3)n[nH]2)C1]

To a solution of cis-benzyl(5-(3-(((4-nitrophenoxy) carbonyl)oxy) cyclopentyl)-1H-pyrazol-3-yl)carbamate (7 g, 15 mmol) in 100 mL THF was added(S)-Butan-2-amine hydrochloride (4.9 g, 45 mmol) and DIEA (9.7 g, 75 mmol). The resulting mixture was stirred at room temperature for 1 h. The resulting mixture was concentrated under reduced pressure, and the residue was purified by silica gel chromatography (PE/EA=2:1) to afford the product (5.2 g, 87% yield). LC-MS (ESI): m/z [M+H]⁺=400.1.

### Step 4: cis-3-(3-amino-1H-pyrazol-5-yl)cyclopentyl((S)-sec-butyl)carbamate

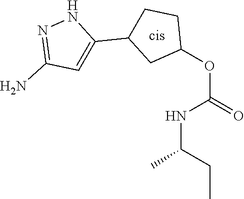 [SMILES: CC[C@H](C)NC(=O)OC1CCC(c2cc(N)n[nH]2)C1]

To a solution of cis-3-(3-(((benzyloxy) carbonyl)amino)-1H-pyrazol-5-yl)cyclopentyl ((S)-sec-butyl)carbamate (4.8 g, 12 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with MeOH (50 mL×3). The filtrate was concentrated under reduced pressure to afford the product (2.6 g, 91% yield). LC-MS (ESI): m/z [M+H]⁺=267.3.

### Step 5: cis-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl((S)-sec-butyl)carbamate

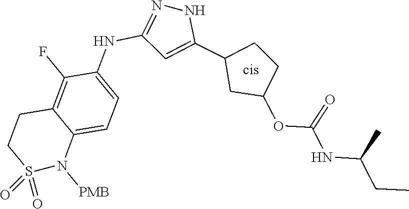

A mixture of 6-bromo-5-fluoro-1-(4-methoxybenzyl)-3,4-dihydro-1H-benzo[c][1,2]thiazine 2,2-dioxide (200 mg, 0.5 mmol), cis-3-(3-amino-1H-pyrazol-5-yl)cyclopentyl ((S)-sec-butyl)carbamate (133 mg, 0.5 mmol), Brettphos Pd G3 (45.2 mg, 0.05 mmol), K₂CO₃ (207 mg, 1.5 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product (114 mg, 39% yield). LC-MS (ESI): m/z [M+H]⁺=586.2.

### Step 6: cis-3-(3-((5-fluoro-2,2-dioxido-3,4-dihydro-1H-benzo[c][1.2]thiazin-6-yl)amino)-1H-pyrazol-5-yl)cyclopentyl((S)-sec-butyl)carbamate

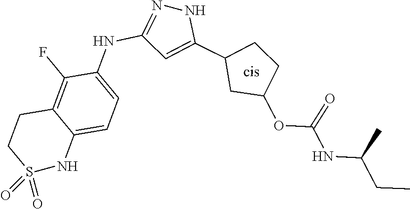 [SMILES: CC[C@H](C)NC(=O)OC1CCC(c2cc(Nc3ccc4c(c3F)CCS(=O)(=O)N4)n[nH]2)C1]

To a flask charged with cis-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl ((S)-sec-butyl)carbamate (114 mg, 0.2 mmol) was added TFA (1 mL) and DCM (3 mL). The result solution was stirred at room temeperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (19.9 mg, 21.5% yield). ¹H NMR (500 MHz, DMSO-d₆) δ 11.68 (s, 1H), 9.76 (s, 1H), 7.85 (s, 1H), 7.80 (s, 1H), 6.88 (d, J=7.9 Hz, 1H), 6.50 (d, J=8.8 Hz, 1H), 5.68 (s, 1H), 4.99 (s, 1H), 3.25 (t, J=6.4 Hz, 4H), 3.10-2.94 (m, 1H), 2.44 (dd, J=13.2, 6.9 Hz, 2H), 2.06-1.95 (m, 1H), 1.91 (d, J=21.7 Hz, 1H), 1.69 (dd, J=20.6, 10.8 Hz, 2H), 1.58 (s, 1H), 1.35 (d, J=5.5 Hz, 2H), 1.00 (d, J=6.4 Hz, 3H), 0.80 (dd, J=13.5, 6.4 Hz, 3H). LC-MS (ESI): m/z [M+H]⁺=466.2.

### Example 462: cis-3-(3-((5-fluoro-2,2-dioxido-3,4-dihydro-1H-benzo[c][1,2]thiazin-6-yl)amino)-1H-pyrazol-5-yl)cyclopentyl(1-methylcyclopropyl)carbamate

### Step 1: cis-benzyl(1-(tert-butyl)-3-(3-(((4-nitrophenoxy) carbonyl)oxy) cyclopentyl)-1H-pyrazol-5-yl)carbamate

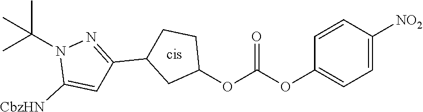 [SMILES: CC(C)(C)n1nc(C2CCC(OC(=O)Oc3ccc([N+](=O)[O-])cc3)C2)cc1NC(=O)OCc1ccccc1]

To a solution of cis-benzyl(1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 2: cis-benzyl(5-(3-(((4-nitrophenoxy) carbonyl)oxy) cyclopentyl)-1H-pyrazol-3-yl)carbamate

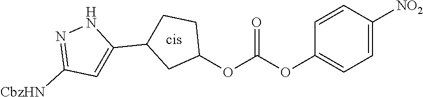 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl(1-(tert-butyl)-3-(3-(((4-nitrophenoxy) carbonyl)oxy) cyclopentyl)-1H-pyrazol-5-yl)carbamate (30 g, 57.5 mmol) was added 100 mL formic acid. The resulting mixture was stirred at 75° C. for over night. The solvent was removed under vacuum to yield 26 g of crude product, which was directly used for the next step without further purification. LC-MS (ESI): m/z [M+H]⁺=467.2.

### Step 3: cis-3-(3-(((benzyloxy) carbonyl)amino)-1H-pyrazol-5-yl)cyclopentyl(1-methylcyclopropyl)carbamate

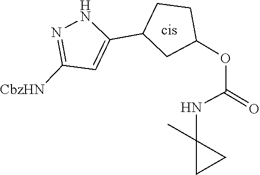 [SMILES: CC1(NC(=O)OC2CCC(c3cc(NC(=O)OCc4ccccc4)n[nH]3)C2)CC1]

To a solution of cis-benzyl(5-(3-(((4-nitrophenoxy) carbonyl)oxy) cyclopentyl)-1H-pyrazol-3-yl)carbamate (10 g, 21.5 mmol) in 100 mL THF was added 1-Methylcyclopropylamine hydrochloride (11.5 g, 107 mmol) and DIEA (27.6 g, 214 mmol). The resulting mixture was stirred at room temperature for 12 h. The resulting mixture was concentrated under reduced pressure and dissolved in EA (200 mL). The mixture was washed with water (200 mL×3), dried with Na₂SO₄, and concentrated. The residue was triturated with MTBE and filtered to afford the product (3.6 g, 42% yield). LC-MS (ESI): m/z [M+H]⁺=399.3.

### Step 4: cis-3-(3-amino-1H-pyrazol-5-yl)cyclopentyl(1-methylcyclopropyl)carbamate

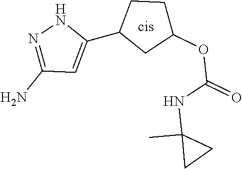 [SMILES: CC1(NC(=O)OC2CCC(c3cc(N)n[nH]3)C2)CC1]

To a solution of cis-3-(3-(((benzyloxy) carbonyl)amino)-1H-pyrazol-5-yl)cyclopentyl(1-methylcyclopropyl)carbamate (3.6 g, 9 mmol) in THF (100 mL) was added Pd/C (10%, wet, 2 g). The suspension was degassed and purged with hydrogen 3 times. The resulting mixture was stirred at room temperature under a hydrogen balloon for 3 h. The mixture was filtered, and the filter cake was washed with EA (200 mL×3). The filtrate was concentrated under reduced pressure to afford the product (2.2 g, 92% yield). LC-MS (ESI): m/z [M+H]⁺=265.3.

### Step 5: cis-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl(1-methylcyclopropyl)carbamate

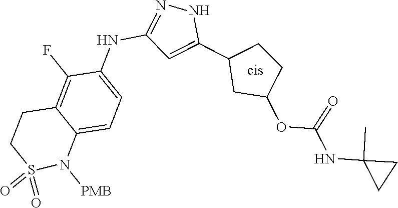

A mixture of 6-bromo-5-fluoro-1-(4-methoxybenzyl)-3,4-dihydro-1H-benzo[c][1,2]thiazine 2,2-dioxide (200 mg, 0.5 mmol), cis-3-(3-amino-1H-pyrazol-5-yl)cyclopentyl(1-methylcyclopropyl)carbamate (132 mg, 0.5 mmol), Brettphos Pd G3 (45.2 mg, 0.05 mmol), K₂CO₃ (207 mg, 1.5 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product (84.5 mg, 29% yield). LC-MS (ESI): m/z [M+H]⁺=584.2.

### Step 6: cis-3-(3-((5-fluoro-2,2-dioxido-3,4-dihydro-1H-benzo[c][1.2]thiazin-6-yl)amino)-1H-pyrazol-5-yl)cyclopentyl(1-methylcyclopropyl)carbamate

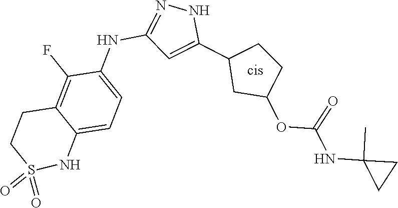 [SMILES: CC1(NC(=O)OC2CCC(c3cc(Nc4ccc5c(c4F)CCS(=O)(=O)N5)n[nH]3)C2)CC1]

To a flask charged with cis-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl(1-methylcyclopropyl)carbamate (84.5 mg, 0.19 mmol) was added TFA (1 mL) and DCM (3 mL). The result solution was stirred at room temeperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (27 mg, 31.2% yield). ¹H NMR (500 MHz, DMSO-d₆) δ 11.71 (s, 1H), 9.77 (s, 1H), 7.82 (s, 2H), 7.34 (s, 1H), 6.50 (d, J=8.8 Hz, 1H), 5.67 (s, 1H), 4.98 (s, 1H), 3.29-3.19 (m, 4H), 3.02 (d, J=7.7 Hz, 1H), 2.46-2.40 (m, 1H), 1.99 (d, J=8.3 Hz, 1H), 1.90 (d, J=19.8 Hz, 1H), 1.68 (s, 2H), 1.55 (s, 1H), 1.23 (s, 3H), 0.57 (d, J=20.6 Hz, 2H), 0.47 (t, J=5.4 Hz, 2H). LC-MS (ESI): m/z [M+H]⁺=464.2.

### Example 463: (1S,3R)-3-(3-((5-fluoro-2,2-dioxido-3,4-dihydro-1H-benzo[c][1,2]thiazin-6-yl)amino)-1H-pyrazol-5-yl)cyclopentyl tert-butylcarbamate

### Step 1: benzyl(1-(tert-butyl)-3-((1R,3S)-3-(((4-nitrophenoxy) carbonyl)oxy) cyclopentyl)-1H-pyrazol-5-yl)carbamate

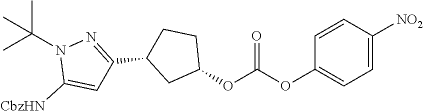 [SMILES: CC(C)(C)n1nc([C@@H]2CC[C@H](OC(=O)Oc3ccc([N+](=O)[O-])cc3)C2)cc1NC(=O)OCc1ccccc1]

To a solution of benzyl benzyl(1-(tert-butyl)-3-((1R,3S)-3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (12.4 g, 85% yield). LC-MS (ESI): m/z [M+H]⁺=523.2.

### Step 2: benzyl(5-((1R,3S)-3-(((4-nitrophenoxy) carbonyl)oxy) cyclopentyl)-1H-pyrazol-3-yl)carbamate

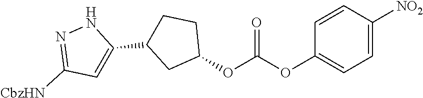

To a round-bottom flask charged with benzyl(1-(tert-butyl)-3-((1R,3S)-3-(((4-nitrophenoxy) carbonyl)oxy) cyclopentyl)-1H-pyrazol-5-yl)carbamate (12.4 g, 23.8 mmol) was added 120 mL formic acid. The resulting mixture was stirred at 75° C. for over night. The solvent was removed under vacuum to yield 11 g of crude product, which was directly used for the next step without further purification. LC-MS (ESI): m/z [M+H]⁺=467.2.

### Step 3: (1S,3R)-3-(3-(((benzyloxy) carbonyl)amino)-1H-pyrazol-5-yl)cyclopentyl tert-butylcarbamate

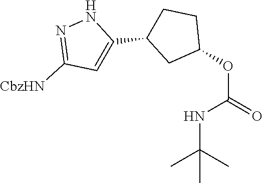 [SMILES: CC(C)(C)NC(=O)O[C@H]1CC[C@@H](c2cc(NC(=O)OCc3ccccc3)n[nH]2)C1]

To a solution of benzyl(5-((1R,3S)-3-(((4-nitrophenoxy) carbonyl)oxy) cyclopentyl)-1H-pyrazol-3-yl)carbamate (10 g, crude) in 100 mL THF was added tert-butylamine (15.7 g, 215 mmol). The resulting mixture was stirred at room temperature for 12 h. The resulting mixture was concentrated under reduced pressure, and the residue was purified by silica gel chromatography (PE/EA=2:1) to afford the product (4.6 g, 53% yield). LC-MS (ESI): m/z [M+H]⁺=401.3.

### Step 4: (1S,3R)-3-(3-amino-1H-pyrazol-5-yl)cyclopentyl tert-butylcarbamate

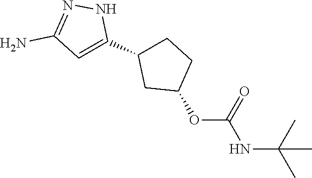 [SMILES: CC(C)(C)NC(=O)O[C@H]1CC[C@@H](c2cc(N)n[nH]2)C1]

To a solution of (1S,3R)-3-(3-(((benzyloxy) carbonyl)amino)-1H-pyrazol-5-yl)cyclopentyl tert-butylcarbamate (4.6 g, 12.6 mmol) in THF (100 mL) was added Pd/C (10%, wet, 2 g). The suspension was degassed and purged with hydrogen 3 times. The resulting mixture was stirred at room temperature under a hydrogen balloon for 5 h. The mixture was filtered, and the filter cake was washed with EA (200 mL×3). The filtrate was concentrated under reduced pressure to afford the product (3.3 g, 98% yield).

LC-MS (ESI): m/z [M+H]⁺=267.3.

### Step 5: (1S,3R)-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl tert-butylcarbamate

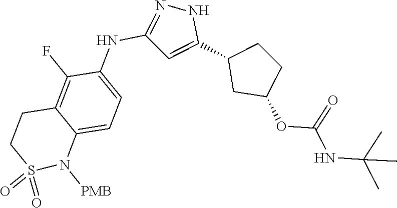

A mixture of 6-bromo-5-fluoro-1-(4-methoxybenzyl)-3,4-dihydro-1H-benzo[c][1,2]thiazine 2,2-dioxide (100 mg, 0.25 mmol), (1S,3R)-3-(3-amino-1H-pyrazol-5-yl)cyclopentyl tert-butylcarbamate (67 mg, 0.25 mmol), Brettphos Pd G3 (22.6 mg, 0.03 mmol), K₂CO₃ (103.5 mg, 0.75 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product (41.5 mg, 28% yield). LC-MS (ESI): m/z [M+H]⁺=586.2.

### Step 6: (1S,3R)-3-(3-((5-fluoro-2,2-dioxido-3,4-dihydro-1H-benzo[c][1,2]thiazin-6-yl)amino)-1H-pyrazol-5-yl)cyclopentyl tert-butylcarbamate

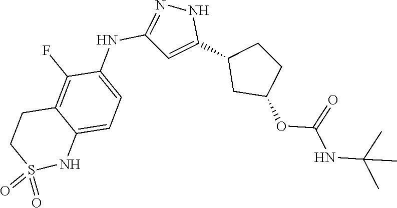 [SMILES: CC(C)(C)NC(=O)O[C@H]1CC[C@@H](c2cc(Nc3ccc4c(c3F)CCS(=O)(=O)N4)n[nH]2)C1]

To a flask charged with (1S,3R)-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl tert-butylcarbamate (41.5 mg, 0.07 mmol) was added TFA (1 mL) and DCM (3 mL). The result solution was stirred at room temeperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (13 mg, 39.9% yield). ¹H NMR (500 MHz, DMSO-d₆) δ 11.70 (s, 1H), 9.77 (s, 1H), 7.83 (s, 2H), 6.75 (s, 1H), 6.50 (d, J=8.8 Hz, 1H), 5.68 (s, 1H), 4.97 (s, 1H), 3.25 (t, J=6.3 Hz, 4H), 3.06-2.93 (m, 1H), 2.48-2.40 (m, 1H), 2.05-1.93 (m, 1H), 1.93-1.83 (m, 1H), 1.77-1.64 (m, 2H), 1.57 (s, 1H), 1.20 (s, 9H). LC-MS (ESI): m/z [M+H]⁺=466.2.

### Example 464: (1R,3S)-3-(3-((5-fluoro-2,2-dioxido-3,4-dihydro-1H-benzo[c][1,2]thiazin-6-yl)amino)-1H-pyrazol-5-yl)cyclopentyl tert-butylcarbamate

### Step 1: benzyl(1-(tert-butyl)-3-((1S,3R)-3-(((4-nitrophenoxy) carbonyl)oxy) cyclopentyl)-1H-pyrazol-5-yl)carbamate

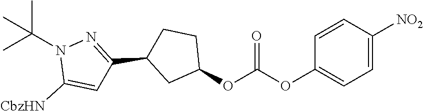 [SMILES: CC(C)(C)n1nc([C@H]2CC[C@@H](OC(=O)Oc3ccc([N+](=O)[O-])cc3)C2)cc1NC(=O)OCc1ccccc1]

To a solution of benzyl benzyl(1-(tert-butyl)-3-((1S,3R)-3-hydroxycyclopentyl)-1H-pyrazol-5-yl)carbamate (10 g, 28.01 mmol) in DCM (100 mL) was added pyridine (11.1 g, 140 mmol), DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.5 g, 78.9% yield). LC-MS (ESI): m/z [M+H]⁺=523.2.

### Step 2: benzyl(5-((1S,3R)-3-(((4-nitrophenoxy) carbonyl)oxy) cyclopentyl)-1H-pyrazol-3-yl)carbamate

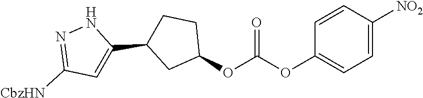 [SMILES: O=C(Nc1cc([C@H]2CC[C@@H](OC(=O)Oc3ccc([N+](=O)[O-])cc3)C2)[nH]n1)OCc1ccccc1]

To a round-bottom flask charged with benzyl(1-(tert-butyl)-3-((1S,3R)-3-(((4-nitrophenoxy) carbonyl)oxy) cyclopentyl)-1H-pyrazol-5-yl)carbamate (20 g, 38 mmol) was added 100 mL formic acid. The resulting mixture was stirred at 75° C. for over night. The solvent was removed under vacuum to yield 19 g of crude product, which was directly used for the next step without further purification. LC-MS (ESI): m/z [M+H]⁺=467.2.

### Step 3: (1R,3S)-3-(3-(((benzyloxy) carbonyl)amino)-1H-pyrazol-5-yl)cyclopentyl tert-butylcarbamate

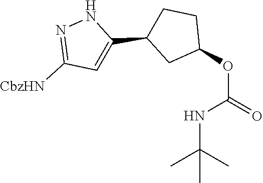 [SMILES: CC(C)(C)NC(=O)O[C@@H]1CC[C@H](c2cc(NC(=O)OCc3ccccc3)n[nH]2)C1]

To a solution of benzyl(5-((1S,3R)-3-(((4-nitrophenoxy) carbonyl)oxy) cyclopentyl)-1H-pyrazol-3-yl)carbamate (5.1 g, 11 mmol) in 100 mL THF was added tert-butylamine (3.9 g, 55 mmol). The resulting mixture was stirred at room temperature for 12 h. The resulting mixture was concentrated under reduced pressure, and the residue was purified by silica gel chromatography (PE/EA=2:1) to afford the product (2.5 g, 56% yield). LC-MS (ESI): m/z [M+H]⁺=401.3.

### Step 4: (1R,3S)-3-(3-amino-1H-pyrazol-5-yl)cyclopentyl tert-butylcarbamate

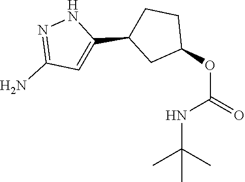 [SMILES: CC(C)(C)NC(=O)O[C@@H]1CC[C@H](c2cc(N)n[nH]2)C1]

To a solution of (1R,3S)-3-(3-(((benzyloxy) carbonyl)amino)-1H-pyrazol-5-yl)cyclopentyl tert-butylcarbamate (2.5 g, 6.1 mmol) in THF (100 mL) was added Pd/C (10%, wet, 1 g). The suspension was degassed and purged with hydrogen 3 times. The resulting mixture was stirred at room temperature under a hydrogen balloon for 5 h. The mixture was filtered, and the filter cake was washed with EA (200 mL×3). The filtrate was concentrated under reduced pressure to afford the product (1.6 g, 95% yield). LC-MS (ESI): m/z [M+H]⁺=267.3.

### Step 5: (1R,3S)-3-(3-((5-fluoro-1-(4-methoxybenzyl)-2,2-dioxido-3,4-dihydro-1H-benzo[c][1.2]thiazin-6-yl)amino)-1H-pyrazol-5-yl)cyclopentyl tert-butylcarbamate

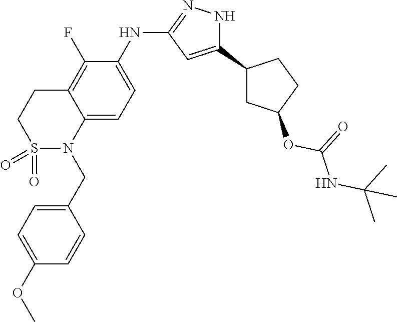 [SMILES: COc1ccc(CN2c3ccc(Nc4cc([C@H]5CC[C@@H](OC(=O)NC(C)(C)C)C5)[nH]n4)c(F)c3CCS2(=O)=O)cc1]

A mixture of 6-bromo-5-fluoro-1-(4-methoxybenzyl)-3,4-dihydro-1H-benzo[c][1,2]thiazine 2,2-dioxide (60 mg, 0.15 mmol), (1R,3S)-3-(3-amino-1H-pyrazol-5-yl)cyclopentyl tert-butylcarbamate (40 mg, 0.15 mmol), Brettphos Pd G3 (18 mg, 0.02 mmol), K₂CO₃ (62 mg, 0.45 mmol) in t-BuOH (20 mL) was stirred at 110° C. for 16 h under N₂ atmosphere. The mixture was concentrated under reduced pressure. The residue was purified by silica gel column chromatography, eluting with EA/PE (10% to 100%) to afford the product (25 mg, 28.4% yield). LC-MS (ESI): m/z [M+H]⁺=586.2.

### Step 6: (1R,3S)-3-(3-((5-fluoro-2,2-dioxido-3,4-dihydro-1H-benzo[c][1.2]thiazin-6-yl)amino)-1H-pyrazol-5-yl)cyclopentyl tert-butylcarbamate

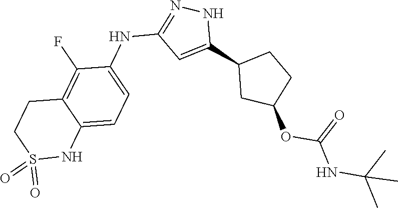 [SMILES: CC(C)(C)NC(=O)O[C@@H]1CC[C@H](c2cc(Nc3ccc4c(c3F)CCS(=O)(=O)N4)n[nH]2)C1]

To a flask charged with (1R,3S)-3-(3-((5-fluoro-1-(4-methoxybenzyl)-2,2-dioxido-3,4-dihydro-1H-benzo[c][1,2]thiazin-6-yl)amino)-1H-pyrazol-5-yl)cyclopentyl tert-butylcarbamate (25 mg, 0.04 mmol) was added TFA (1 mL) and DCM (3 mL). The result solution was stirred at room temeperature for 2 h. The solvent was removed under vacuum and the residue was purified by prep HPLC (Waters XSelect C18: RD-CO-094 column, eluting with a gradient of acetonitrile/water containing 0.1% FA, 20%-40%) to afford the product (9 mg, 48.3% yield). ¹H NMR (500 MHz, DMSO-d₆) δ 11.70 (s, 1H), 9.77 (s, 1H), 7.83 (s, 2H), 6.75 (s, 1H), 6.50 (d, J=8.8 Hz, 1H), 5.68 (s, 1H), 4.97 (s, 1H), 3.25 (t, J=6.3 Hz, 4H), 3.06-2.93 (m, 1H), 2.48-2.40 (m, 1H), 2.05-1.93 (m, 1H), 1.93-1.83 (m, 1H), 1.77-1.64 (m, 2H), 1.57 (s, 1H), 1.20 (s, 9H). LC-MS (ESI): m/z [M+H]⁺=466.2.

### Example 465: cis-3-(3-((4-fluoro-2,2-dioxido-1,3-dihydrobenzo[c][1,2,5]thiadiazol-5-yl)amino)-1H-pyrazol-5-yl)cyclopentyl isopropylcarbamate

### Step 1:4-bromo-3-fluorobenzene-1,2-diamine

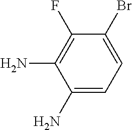

To a mixture of 4-bromo-3-fluoro-2-nitroaniline (7.3 g, 31.2 mmol) in MeOH (30 mL) were added (5.2 g, 93.6 mmol) and HCl (12N, 10 mL). The mixture was stirred at rt for 16 h. The mixture was concentrated under reduced pressure. The mixture was quenched by NaHCO₃ (100 mL), extracted with EA (3×100 mL). The combined organic layers were dried over Na₂SO₄, filtered and concentrated under vacuum. The residue was purified by silica gel column chromatography, eluting with EA/PE (0-30%) to afford (3 g, 46.9%). LC-MS (ESI): m/z [M+H]⁺=205.

### Step 2:5-bromo-4-fluoro-1,3-dihydrobenzo[c][1,2,5]thiadiazole 2,2-dioxide

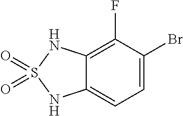 [SMILES: O=S1(=O)Nc2ccc(Br)c(F)c2N1]

To a mixture of 4-bromo-3-fluorobenzene-1,2-diamine (410 mg, 2 mmol) in pyridine (10 mL) was added Sulfamide (538 mg, 5.6 mmol). The mixture was stirred at 120° C. for 16 h. The mixture was concentrated under reduced pressure. The residue was purified by silica gel column chromatography, eluting with EA/PE (0-40%) to afford (320 mg, 59.8%). LC-MS (ESI): m/z [M+H]⁺=267.

### Step 3:5-bromo-4-fluoro-1,3-bis(4-methoxybenzyl)-1,3-dihydrobenzo[c][1.2.5]thiadiazole 2,2-dioxide

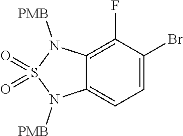

To a mixture of 5-bromo-4-fluoro-1,3-dihydrobenzo[c][1,2,5]thiadiazole 2,2-dioxide (200 mg, 0.76 mmol) and K₂CO₃ (208 mg, 1.51 mmol) in DMF was added 1-(chloromethyl)-4-methoxybenzene (236.5 mg, 1.51 mmol). The mixture was stirred at 50° C. for 5 h. The mixture was quenched by water (50 mL), extracted with EA (3×50 mL). The combined organic layers were dried over Na₂SO₄, filtered and concentrated under vacuum. The residue was purified by silica gel column chromatography, eluting with EA/PE (0-45%) to afford the product (230 mg, 60%). LC-MS (ESI): m/z [M+Na]⁺=529.

### Step 4: cis-3-(3-((4-fluoro-1,3-bis(4-methoxybenzyl)-2,2-dioxido-1,3-dihydrobenzo[c][1.2.5]thiadiazol-5-yl)amino)-1H-pyrazol-5-yl)cyclopentyl isopropylcarbamate

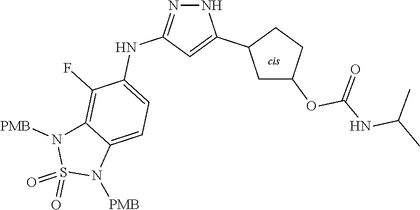

A mixture of 5-bromo-4-fluoro-1,3-bis(4-methoxybenzyl)-1,3-dihydrobenzo[c][1,2,5]thiadiazole 2,2-dioxide (70 mg, 0.14 mmol), cis-3-(3-amino-1H-pyrazol-5-yl)cyclopentyl isopropylcarbamate (25 mg, 0.097 mmol), Brettphos Pd G3 (25 mg, 0.097 mmol) and K₂CO₃ (58 mg, 0.42 mmol) in t-BuOH (5 mL) was stirred at 110° C. for 16 h under a nitrogen atmosphere. The mixture was concentrated under vacuum. The residue was purified by silica gel column chromatography, eluting with EA/PE (0-100%) to afford the product (50 mg, 53.3%). LC-MS (ESI): m/z [M+H]⁺=679.

### Step 5: cis-3-(3-((4-fluoro-2,2-dioxido-1,3-dihydrobenzo[c][1.2.5]thiadiazol-5-yl)amino)-1H-pyrazol-5-yl)cyclopentyl isopropylcarbamate

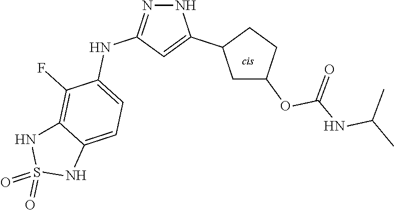 [SMILES: CC(C)NC(=O)OC1CCC(c2cc(Nc3ccc4c(c3F)NS(=O)(=O)N4)n[nH]2)C1]

A mixture of cis-3-(3-((4-fluoro-1,3-bis(4-methoxybenzyl)-2,2-dioxido-1,3-dihydrobenzo[c][1,2,5]thiadiazol-5-yl)amino)-1H-pyrazol-5-yl)cyclopentyl isopropylcarbamate (50 mg, 0.074 mmol) in TFA (5 mL) was stirred at rt for 16 h. The mixture was concentrated under vacuum. The residue was purified by Prep-HPLC to the product (10.26 mg, 31.7%). ¹H NMR (500 MHz, DMSO-d₆) δ 11.66 (s, 1H), 11.16 (s, 1H), 10.66 (s, 1H), 7.76 (s, 1H), 7.55-7.37 (m, 1H), 6.99-6.86 (m, 1H), 6.53 (d, J=10 Hz, 1H), 5.65 (s, 1H), 5.05-4.92 (m, 1H), 3.63-3.51 (m, 1H), 3.08-2.95 (m, 1H), 2.46-2.41 (m, 1H), 2.04-1.95 (m, 1H), 1.94-1.83 (m, 1H), 1.77-1.63 (m, 2H), 1.62-1.53 (m, 1H), 1.07-0.97 (m, 6H). LC-MS (ESI): m/z [M+H]⁺=439.

### Example 466: cis-3-(3-((4-fluoro-3-methyl-2,2-dioxido-1,3-dihydrobenzo[c][1,2,5]thiadiazol-5-yl)amino)-1H-pyrazol-5-yl)cyclopentyl isopropylcarbamate

### Step 1:4-bromo-3-fluoro-N-(4-methoxybenzyl)-2-nitroaniline

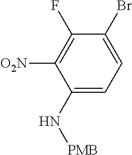

To a mixture of 4-bromo-3-fluoro-2-nitroaniline (4.68 g, 20 mmol), K₂CO₃ (6.6 g, 48 mmol) and KI (3.32 g, 20 mmol) in ACN (100 mL) was added 1-(chloromethyl)-4-methoxybenzene (3.76 g, 24 mmol). The mixture was stirred at 70° C. for 6 h. The mixture was filtered and the filtrate was concentrated under vacuum. The residue was purified by silica gel column chromatography, eluting with EA/PE (0-35%) to afford the product (6.1 g, 86%). LC-MS (ESI): m/z [M+Na]⁺=377.

### Step 2:4-bromo-3-fluoro-N1-(4-methoxybenzyl)benzene-1,2-diamine

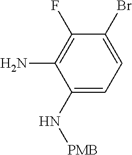

To a solution of 4-bromo-3-fluoro-N-(4-methoxybenzyl)-2-nitroaniline (1 g, 2.82 mmol) in MeOH (18 mL) and H₂O (2 mL) were added NH₄Cl (1.51 g, 28.2 mmol) and Fe powder (788 mg, 14.1 mmol). The mixture was stirred at 80° C. for 6 h. The mixture was filtered and the filtrate was concentrated under vacuum. The residue was purified by silica gel column chromatography, eluting with EA/PE (0-40%) to afford the product (460 mg, 50.2%). LC-MS (ESI): m/z [M+Na]⁺=347.

### Step 3:5-bromo-4-fluoro-1-(4-methoxybenzyl)-1,3-dihydrobenzo[c][1 2.5]thiadiazole 2,2-dioxide

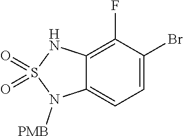

To a mixture of 4-bromo-3-fluoro-N1-(4-methoxybenzyl)benzene-1,2-diamine (460 mg, 1.4 mmol) in pyridine (10 mL) was added Sulfamide (376 mg, 3.9 mmol). The mixture was stirred at 120° C. for 16 h. The mixture was concentrated under reduced pressure. The residue was purified by silica gel column chromatography, eluting with EA/PE (0-30%) to afford (228 mg, 42%). LC-MS (ESI): m/z [M+H]⁺=387.

### Step 4:5-bromo-4-fluoro-1-(4-methoxybenzyl)-3-methyl-1,3-dihydrobenzo[c] [1.2.5]thiadiazole 2,2-dioxide

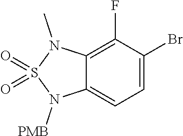

To a mixture of 5-bromo-4-fluoro-1-(4-methoxybenzyl)-1,3-dihydrobenzo[c][1,2,5]thiadiazole 2,2-dioxide (228 mg, 0.63 mmol) and K₂CO₃ (80 mg, 0.63 mmol) in DMF was added iodomethane (90 mg, 0.63 mmol). The mixture was stirred at 50° C. for 5 h. The mixture was quenched by water (50 mL), extracted with EA (3×50 mL). The combined organic layers were dried over Na₂SO₄, filtered and concentrated under vacuum. The residue was purified by silica gel column chromatography, eluting with EA/PE (0-45%) to afford the product (162 mg, 61%). LC-MS (ESI): m/z [M+Na]⁺=423.

### Step 5: cis-3-(3-((4-fluoro-1-(4-methoxybenzyl)-3-methyl-2,2-dioxido-1,3-dihydrobenzo[c][1.2.5]thiadiazol-5-yl)amino)-1H-pyrazol-5-yl)cyclopentyl isopropylcarbamate

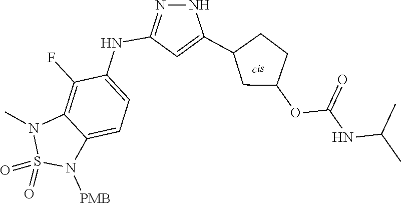

The titled compound was synthesized in the procedures similar to Example 465. LC-MS (ESI): m/z [M+H]⁺=573.2.

### Step 6: cis-3-(3-((4-fluoro-3-methyl-2,2-dioxido-1,3-dihydrobenzo[c][1,2,5]thiadiazol-5-yl)amino)-1H-pyrazol-5-yl)cyclopentyl isopropylcarbamate

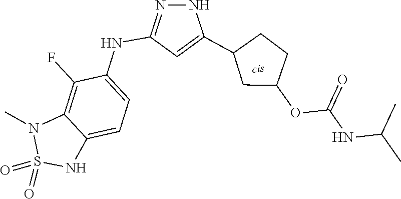 [SMILES: CC(C)NC(=O)OC1CCC(c2cc(Nc3ccc4c(c3F)N(C)S(=O)(=O)N4)n[nH]2)C1]

The titled compound was synthesized in the procedures similar to Example 465. ¹H NMR (500 MHz, DMSO-d₆) δ 11.71 (s, 1H), 11.04 (s, 1H), 8.13 (s, 1H), 7.60-7.43 (m, 1H), 6.94 (d, J=10 Hz, 1H), 6.58 (d, J=10 Hz, 1H), 5.68 (s, 1H), 5.04-4.93 (m, 1H), 3.63-3.51 (m, 1H), 3.27 (s, 3H), 3.08-2.96 (m, 1H), 2.47-2.40 (m, 1H), 2.05-1.95 (m, 1H), 1.94-1.83 (m, 1H), 1.79-1.64 (m, 2H), 1.62-1.52 (m, 1H), 1.08-0.97 (m, 6H). LC-MS (ESI): m/z [M+H]⁺=453.2.

### Example 467: cis-3-(3-((4-fluoro-1-methyl-2,2-dioxido-1,3-dihydrobenzo[c][1,2,5]thiadiazol-5-yl)amino)-1H-pyrazol-5-yl)cyclopentyl isopropylcarbamate

### Step 1:4-bromo-3-fluoro-N-methyl-2-nitroaniline

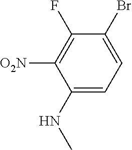

To a mixture of 4-bromo-3-fluoro-2-nitroaniline (4.68 g, 20 mmol) in DMF (100 mL) was added NaH (800 mg, 20 mmol, 60%) at 0° C. and stirred at this temperature for 1h. Iodomethane (2.8 g, 20 mmol) was added. The mixture was stirred at room temperature for 6 h. The mixture was quenched by aq. NH₄Cl and extracted with EA (3×200 mL). The EA layers were combined, washed with brine, dried with Na₂SO₄ and concentrated under vacuum. The residue was purified by silica gel column chromatography, eluting with EA/PE (0-35%) to afford the product (3.7 g, 75%). LC-MS (ESI): m/z [M+H]⁺=249.

### Step 2:4-bromo-3-fluoro-N1-methylbenzene-1,2-diamine

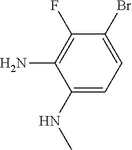

To a solution of 4-bromo-3-fluoro-N-methyl-2-nitroaniline (3.7 g, 15 mmol) in EtOH (100 mL) and H₂O (20 mL) were added NH₄Cl (4 g, 75 mmol) and Fe powder (4.2 g, 75 mmol). The mixture was stirred at 80° C. for 6 h. The mixture was filtered and the filtrate was concentrated under vacuum. The residue was purified by silica gel column chromatography, eluting with EA/PE (0-40%) to afford the product (1.6 g, 49%). LC-MS (ESI): m/z [M+H]⁺=219.

### Step 3:5-bromo-4-fluoro-1-methyl-1,3-dihydrobenzo[c][1,2,5]thiadiazole 2,2-dioxide

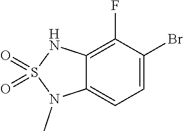

To a mixture of 4-bromo-3-fluoro-N1-methylbenzene-1,2-diamine (1.6 g, 7.5 mmol) in pyridine (100 mL) was added Sulfamide (2 g, 21 mmol). The mixture was stirred at 120° C. for 16 h. The mixture was concentrated under reduced pressure. The residue was purified by silica gel column chromatography, eluting with EA/PE (0-30%) to afford (1 g, 48%). LC-MS (ESI): m/z [M+H]⁺=281.

### Step 4:5-bromo-4-fluoro-3-(4-methoxybenzyl)-1-methyl-1,3-dihydrobenzo[c][1 2.5]thiadiazole 2,2-dioxide

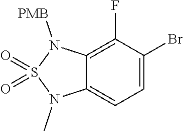

To a mixture of 5-bromo-4-fluoro-1-methyl-1,3-dihydrobenzo[c][1,2,5]thiadiazole 2,2-dioxide (1 g, 3.6 mmol) and K₂CO₃ (208 mg, 3.6 mmol) in DMF was added 1-(chloromethyl)-4-methoxybenzene (565 mg, 3.6 mmol). The mixture was stirred at 50° C. for 5 h. The mixture was quenched by water (50 mL), extracted with EA (3×50 mL). The combined organic layers were combined, dried over Na₂SO₄, filtered and concentrated under vacuum. The residue was purified by silica gel column chromatography, eluting with EA/PE (0-45%) to afford the product (1 g, 71%). LC-MS (ESI): m/z [M+Na]⁺=423.

### Step 5: cis-3-(3-((4-fluoro-3-(4-methoxybenzyl)-1-methyl-2,2-dioxido-1,3-dihydrobenzo[c][1.2.5]thiadiazol-5-yl)amino)-1H-pyrazol-5-yl)cyclopentyl isopropylcarbamate

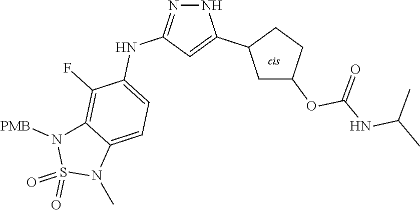

The titled compound was synthesized in the procedures similar to Example 466. LC-MS (ESI): m/z [M+H]⁺=573.2.

### Step 6: cis-3-(3-((4-fluoro-1-methyl-2,2-dioxido-1,3-dihydrobenzo[c][1,2,5]thiadiazol-5-yl)amino)-1H-pyrazol-5-yl)cyclopentyl isopropylcarbamate

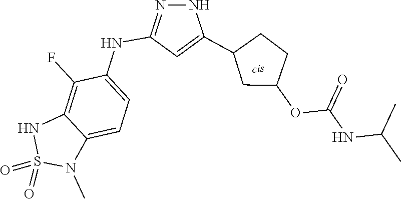 [SMILES: CC(C)NC(=O)OC1CCC(c2cc(Nc3ccc4c(c3F)NS(=O)(=O)N4C)n[nH]2)C1]

The titled compound was synthesized in the procedures similar to Example 466. ¹H NMR (500 MHz, DMSO-d₆) δ 11.88-11.40 (m, 2H), 7.78 (s, 1H), 7.62-7.41 (m, 1H), 6.94 (d, J=10 Hz, 1H), 6.62 (d, J=10 Hz, 1H), 5.65 (s, 1H), 5.04-4.92 (m, 1H), 3.61-3.53 (m, 1H), 3.08 (s, 3H), 3.05-2.97 (m, 1H), 2.47-2.39 (m, 1H), 2.03-1.95 (m, 1H), 1.94-1.82 (m, 1H), 1.77-1.64 (m, 2H), 1.62-1.52 (m, 1H), 1.07-0.97 (m, 6H). LC-MS (ESI): m/z [M+H]⁺=453.2.

### CDK2/Cyclin E1 and CDK1/Cyclin B1 Biochemical Assays

Compounds disclosed herein were tested for inhibition of CDK2/Cyclin E1 or CDK1/Cyclin B1 kinase in an assay based on the time-resolved fluorescence-resonance energy transfer (TR-FRET) methodology. The assay was carried out in 384-well low volume black plates in a reaction mixture containing CDK2/Cyclin E1 or CDK1/Cyclin B1, 0.060 mM ATP for CDK2/Cyclin E1 or 0.015 mM ATP for CDK1/Cyclin B1, 0.15 μM Rb (Ser780)-biotin substrate and 0-10 μM compound in buffer containing 50 mM HEPES pH7.0, 0.02% NaN₃, 0.01% BSA, 0.1 mM Orthovanadate, 50 mM MgCl₂, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK2/Cyclin E1 or CDK1/Cyclin B1 kinase activity in presence of increasing concentrations of compounds was calculated based on the ratio of fluorescence at 665 nm to that at 620 nm. The IC50 for each compound was derived from fitting the data to the four-parameter logistic equation by Dotmatics. The IC50 data as measured for the Examples are shown in Table 1.

TABLE 1

| Example | CDK2/Cyclin E1 IC₅₀ (nM) | CDK1/Cyclin B1 IC₅₀ (nM) |
|---|---|---|
| 1a | 22.0 | 860.0 |
| 1b | 18.0 | 259.0 |
| 2 | 0.9 | 36.1 |
| 3 | 1.2 | 29.0 |
| 4 | 8.6 | 184.0 |
| 5a | 2.2 | 49.0 |
| 5b | 1.0 | 14.0 |
| 5c | 4.2 | 95.0 |
| 5d | 2.9 | 45.0 |
| 6 | 6.5 | 108.0 |
| 7a | 0.9 | 28.0 |
| 7b | 0.9 | 17 |
| 8 | 121.0 | 1541.0 |
| 9a | 4.5 | 375.0 |
| 9b | 2.5 | 94.0 |
| 10 | 102.0 | 3018.0 |
| 11 | 4.1 | 140.0 |
| 12a | 10.0 | 921.0 |
| 12b | 9.3 | 359.0 |
| 13 | 21.0 | 563.0 |
| 14 | 30.0 | 1088.0 |
| 15 | 23.0 | 1066.0 |
| 16 | 18.0 | 385.1 |
| 17 | 11.8 | 470.5 |
| 18 | 21.5 | 422.0 |
| 19 | 305.0 | 1072.0 |
| 20 | 931 | >10000 |
| 21 | >10000 | >10000 |
| 22 | 202.0 | >10000 |
| 23 | 244.0 | 1175.0 |
| 24 | 887.0 | 9963.0 |
| 25 | 11.0 | 271.0 |
| 26 | 2.0 | 60.0 |
| 27 | 33.0 | 958.0 |
| 28 | 1255.0 | >10000 |
| 29 | 2.2 | 113.0 |
| 30 | 7.2 | 544.0 |
| 31 | 7.0 | 166.0 |
| 32 | 21.0 | 424.0 |
| 33 | 1.8 | 38.0 |
| 34 | 1.9 | 81.0 |
| 35 | 8.9 | 296.0 |
| 36 | 18.0 | 329.0 |
| 37 | 7.2 | 313.0 |
| 38a | 38.0 | 1496.0 |
| 38b | 13.0 | 279.0 |
| 39 | 15.0 | 374.0 |
| 39a | 49 | 1365 |
| 39b | 26 | 390 |
| 40 | 2.3 | 154.0 |
| 40a | 5.4 | 370 |
| 40b | 3.1 | 181 |
| 41 | 2.3 | 512.0 |
| 42a | 14.0 | 891.0 |
| 42b | 5.5 | 128.0 |
| 43 | 1.1 | 130.0 |
| 44a | 1.3 | 138.0 |
| 44b | 2.5 | 91.0 |
| 45 | 181.0 | 6013.0 |
| 46 | 230.0 | 4423.0 |
| 47 | 158.0 | 7379.0 |
| 48 | 243.0 | >10000 |
| 49 | 758.0 | >10000 |
| 50 | 5.6 | 140.0 |
| 51a | 2.5 | 194.0 |
| 51b | 3.3 | 82.0 |
| 51c | 2.1 | 106.0 |
| 51d | 8.7 | 447.0 |
| 52a | 3.5 | 274.0 |
| 52b | 2.7 | 153.0 |
| 52c | 1.9 | 154.0 |
| 52d | 1.0 | 56.0 |
| 53a | 6.3 | 367.0 |
| 53b | 3.8 | 92.0 |
| 53c | 15.0 | 686.0 |
| 53d | 7.1 | 170.0 |
| 54a | 1.1 | 309.0 |
| 54b | 2.7 | 564.0 |
| 54c | 0.3 | 40.0 |
| 54d | 3.2 | 428.0 |
| 55 | 71.0 | 1890.0 |
| 56 | 25.0 | 472.0 |
| 57 | 48.0 | 1081.0 |
| 58 | 93.0 | 694.0 |
| 59 | 5.8 | 296.0 |
| 60 | 9.2 | 497.0 |
| 61 | 10.0 | 836.0 |
| 62 | 9.5 | 758.0 |
| 63 | 23.0 | 1114.0 |
| 64 | 4.6 | 214.0 |
| 65 | 6.9 | 299.0 |
| 66 | 7.3 | 412.0 |
| 67 | 14.0 | 472.0 |
| 68 | 15.0 | 1180.0 |
| 69 | 9.5 | 647.0 |
| 70 | 11.0 | 503.0 |
| 71 | 5.2 | 440.0 |
| 72 | 5911.0 | >10000 |
| 73 | 7.9 | 583.0 |
| 74 | 16.0 | 962.0 |
| 75 | 16.0 | 1077.0 |
| 76 | 10.0 | 416.0 |
| 77 | 10.0 | 621.0 |
| 78 | 8.9 | 556.0 |
| 79 | 8.3 | 638.0 |
| 80 | 10.0 | 767.0 |
| 81 | 7.2 | 537.0 |
| 82 | 10.0 | 381.0 |
| 83 | 15.0 | 565.0 |
| 84 | 8.6 | 390.0 |
| 85 | 4.6 | 470.0 |
| 86 | 7.8 | 735.0 |
| 87 | 29.0 | 1091.0 |
| 88 | 10.0 | 1010.0 |
| 89 | 34.0 | 879.0 |
| 90 | 20.0 | 762.0 |
| 91 | 24.0 | 945.0 |
| 92 | 36.0 | 1547.0 |
| 93 | 6.3 | 531.0 |
| 94 | 332.0 | >10000 |
| 95 | 15.0 | 972.0 |
| 96 | 36.0 | 2017.0 |
| 97 | 5.2 | 449.0 |
| 98 | 20.0 | 1911.0 |
| 99 | 12.0 | 529.0 |
| 100 | 5.2 | 236.0 |
| 101 | 10.0 | 1025.0 |
| 102 | 5.1 | 406.0 |
| 103 | 3.8 | 350.0 |
| 104 | 5.7 | 244.0 |
| 105 | 8.4 | 689.0 |
| 106 | 6.0 | 631.0 |
| 107 | 9.6 | 532.0 |
| 108 | 37.0 | >10000 |
| 109 | 11.0 | 1190.0 |
| 110 | 2.7 | 225.0 |
| 111 | 2.1 | 225.0 |
| 112 | 6.3 | 458.0 |
| 113 | 3.6 | 288.0 |
| 114 | 9.6 | 596.0 |
| 115 | 6.0 | 407.0 |
| 116a | 5.4 | 518.0 |
| 116b | 5.4 | 189.0 |
| 117 | 9.6 | 431.0 |
| 117a | 2.5 | 326 |
| 117b | 1.8 | 91 |
| 118 | 3.1 | 345.0 |
| 118a | 14 | 395 |
| 118b | 8 | 168 |
| 119 | 2.7 | 146.0 |
| 119a | 2.5 | 216 |
| 119b | 1.9 | 114 |
| 120 | 32.0 | 773.0 |
| 121a | 5.8 | 425.0 |
| 121b | 3.3 | 130.0 |
| 122a | 12.0 | 455.0 |
| 122b | 8.8 | 124.0 |
| 123a | 5.2 | 613.0 |
| 123b | 2.6 | 219.0 |
| 124 | 1.6 | 120.0 |
| 125a | 12.0 | 1220.0 |
| 125b | 4.9 | 341.0 |
| 126 | 8.3 | 261.0 |
| 127 | 1.9 | 176.0 |
| 128 | 1.8 | 98.0 |
| 128a | 2.7 | 192 |
| 128b | 2.5 | 96 |
| 129a | 4.7 | 652.0 |
| 129b | 2.6 | 185.0 |
| 130 | 5.7 | 241.0 |
| 131 | 1.4 | 152.0 |
| 132 | 1.6 | 104.0 |
| 132a | 2.2 | 206 |
| 132b | 1.9 | 65 |
| 133a | 24.0 | 1083.0 |
| 133b | 8.6 | 456.0 |
| 134a | 3.8 | 488.0 |
| 134b | 1.3 | 120.0 |
| 135 | 4.3 | 320.0 |
| 136 | 22.0 | 457.0 |
| 137 | 35.0 | 375.0 |
| 138a | 2.6 | 237.0 |
| 138b | 1.5 | 60.0 |
| 139a | 1.2 | 223.0 |
| 139b | 0.9 | 52.0 |
| 140a | 1.1 | 97.0 |
| 140b | 1.4 | 38.0 |
| 141 | 8.0 | 157.0 |
| 142 | 1.2 | 46.0 |
| 143 | 4.9 | 242.0 |
| 144 | 3.9 | 302.0 |
| 145 | 14.0 | 267.0 |
| 146 | 8.1 | 323.0 |
| 147 | 9.1 | 606 |
| 148 | 44 | 523 |
| 149 | 42 | 666 |
| 150 | 0.3 | 24.0 |
| 151 | 80.0 | 668.0 |
| 152 | 3.6 | 324.0 |
| 153 | 91.0 | 1120.0 |
| 154 | 1.7 | 333.0 |
| 154a | 2.3 | 187 |
| 154b | 3.5 | 69 |
| 155 | 1.3 | 13.0 |
| 156 | 1.7 | 76.0 |
| 157 | 15.0 | 240.0 |
| 158 | 3.1 | 123.0 |
| 158a | 5.3 | 799 |
| 158b | 3.3 | 248 |
| 159 | 1.0 | 30.0 |
| 160 | 105.0 | 579.0 |
| 161 | 6.5 | 332.0 |
| 162 | 2.4 | 163.0 |
| 162a | 3.4 | 456 |
| 162b | 1.1 | 186 |
| 162c | 3.1 | 408 |
| 162d | 3.7 | 158 |
| 163 | 0.8 | 18.0 |
| 164 | 5.0 | 294.0 |
| 164a | 4.3 | 364 |
| 164b | 8.4 | 189 |
| 165 | 1.3 | 49.0 |
| 166 | 2.9 | 74.0 |
| 167 | 9.2 | 514.0 |
| 168 | 12.0 | 545.0 |
| 169 | 7.4 | 471.0 |
| 170 | 23.0 | 407.0 |
| 171 | 10.0 | 183.0 |
| 172 | 0.7 | 14.0 |
| 173 | 6.7 | 73.0 |
| 174 | 6.5 | 136.0 |
| 175 | 10.0 | 359.0 |
| 176 | 8.0 | 230.0 |
| 177 | 1.1 | 27.0 |
| 178 | 1.5 | 41.0 |
| 179 | 0.3 | 5.5 |
| 180 | 2.9 | 102.0 |
| 181 | 58.0 | 1251.0 |
| 182 | 279.0 | 5581.0 |
| 183 | 1395.0 | 7567.0 |
| 184 | 1.5 | 4.1 |
| 185 | 239.0 | 1425.0 |
| 186a | 4.8 | 255.0 |
| 186b | 1.7 | 44.0 |
| 187 | 0.7 | 14.0 |
| 188 | 0.8 | 29.0 |
| 189a | 10.0 | 350.0 |
| 189b | 2.7 | 41.0 |
| 190 | 8.1 | 328.0 |
| 191 | 9.3 | 191.0 |
| 192 | 6.0 | 116.0 |
| 193 | 16.0 | 362.0 |
| 194 | 3.7 | 86.0 |
| 195a | 12.0 | 73.0 |
| 195b | 17.0 | 67.0 |
| 196 | 4.3 | 47.0 |
| 197 | 12.0 | 119.0 |
| 198 | 18.0 | 75.0 |
| 199 | 5.5 | 84.0 |
| 200 | 5.5 | 75.0 |
| 201 | 11.0 | 130.0 |
| 202 | 6.2 | 98.0 |
| 203 | 5.4 | 41.0 |
| 204 | 1.8 | 51.0 |
| 205 | 7.5 | 41.0 |
| 206 | 6.4 | 63.0 |
| 207 | 6.6 | 35.0 |
| 208 | 16.0 | 262.0 |
| 209 | 5.4 | 37.0 |
| 210 | 2.1 | 40.0 |
| 211 | 8.4 | 197.0 |
| 212 | 3.5 | 60.0 |
| 213 | 2.7 | 30.0 |
| 214 | 1.7 | 44.0 |
| 215 | 3.1 | 30.0 |
| 216 | 7.8 | 54.0 |
| 217 | 8.8 | 126.0 |
| 218 | 112.0 | 999.0 |
| 219a | 5.4 | 46.0 |
| 219b | 3.6 | 28.0 |
| 220a | 2.6 | 56.0 |
| 220b | 2.6 | 35.0 |
| 221 | 8.6 | 49.0 |
| 222 | 5.0 | 48.0 |
| 223 | 5.5 | 35.0 |
| 224 | 29.0 | 2579.0 |
| 225 | 87.0 | 789.0 |
| 226 | 2.0 | 77.0 |
| 227 | 5.0 | 249.0 |
| 227a | 3.5 | 156 |
| 227b | 1.3 | 45 |
| 228 | 9.4 | 123.0 |
| 229 | 6.4 | 30.0 |
| 230 | 9.8 | 78.0 |
| 231 | 1.5 | 13.0 |
| 232 | 12.0 | 27.0 |
| 233 | 1.3 | 10.0 |
| 234 | 4.2 | 67.0 |
| 235 | 12.0 | 232.0 |
| 236 | 47.0 | 320.0 |
| 237 | 46.0 | 435.0 |
| 238 | 16.0 | 153.0 |
| 239 | 9.6 | 158.0 |
| 240 | 3.0 | 28.0 |
| 241 | 128.0 | 4471.0 |
| 242 | 5.1 | 70.0 |
| 243 | 37.0 | 978.0 |
| 244 | 1056.0 | 7265.0 |
| 245 | 44.0 | 941.0 |
| 246a | 3.4 | 201.0 |
| 246b | 2.3 | 68.0 |
| 247 | 9.7 | 79.0 |
| 248 | 2.6 | 49.0 |
| 249 | 2.1 | 29.0 |
| 249a | 1.1 | 31 |
| 249b | 0.6 | 12 |
| 250 | 2.1 | 30.0 |
| 251 | 0.4 | 25.0 |
| 251a | 1.1 | 74 |
| 251b | 1 | 31 |
| 252 | 3.8 | 79.0 |
| 253 | 4.3 | 96.0 |
| 254 | 10.0 | 249.0 |
| 255a | 2.9 | 133.0 |
| 255b | 5.2 | 149.0 |
| 256a | 5.3 | 334.0 |
| 256b | 4.0 | 130.0 |
| 257 | 1.5 | 33.0 |
| 258 | 1.5 | 37.0 |
| 259a | 5.4 | 364.0 |
| 259b | 3.0 | 157.0 |
| 260 | 3.2 | 110.0 |
| 260a | 5.9 | 270 |
| 260b | 3.4 | 103 |
| 261 | 1.3 | 49.0 |
| 26la | 1.3 | 119 |
| 261b | 0.7 | 23 |
| 262 | 1.3 | 33.0 |
| 263a | 2.1 | 70.0 |
| 263b | 5.1 | 91.0 |
| 264a | 1.3 | 65.0 |
| 264b | 1.1 | 22.0 |
| 265a | 1.6 | 41.0 |
| 265b | 2.4 | 26.0 |
| 266a | 3.3 | 96 |
| 266b | 3.5 | 59 |
| 267a | 7.8 | 142.0 |
| 267b | 6.4 | 73.0 |
| 268a | 14.0 | 285.0 |
| 268b | 4.0 | 75.0 |
| 269 | 5.6 | 61.0 |
| 270 | 1.7 | 37.0 |
| 27la | 3.2 | 172.0 |
| 271b | 1.8 | 49.0 |
| 272a | 15.5 | 146.0 |
| 272b | 3.0 | 59.0 |
| 273 | 2.5 | 43.0 |
| 274 | 1.0 | 22.0 |
| 275a | 8.4 | 398.0 |
| 275b | 3.1 | 160.0 |
| 276 | 5.0 | 111.0 |
| 277 | 1.7 | 36.0 |
| 278 | 1.3 | 56.0 |
| 279 | 0.7 | 43.0 |
| 280 | 10.0 | 149.0 |
| 281 | 8.1 | 131.0 |
| 282 | 7.7 | 141.0 |
| 283 | 3.0 | 56.0 |
| 284 | 8.3 | 61.0 |
| 285 | 12.0 | 196.0 |
| 286 | 11.0 | 174.0 |
| 287 | 7.7 | 77.0 |
| 288 | 5.5 | 152.0 |
| 289a | 10.0 | 196.0 |
| 289b | 12.0 | 116.0 |
| 290a | 16.0 | 356.0 |
| 290b | 7.5 | 94.0 |
| 29la | 2.0 | 76.0 |
| 291b | 1.3 | 48.0 |
| 292a | 13.0 | 1410.0 |
| 292b | 5.2 | 457.0 |
| 293 | 64.0 | 1120.0 |
| 294 | 15.0 | 359.0 |
| 295 | 23.0 | 426.0 |
| 296a | 15.0 | 800.0 |
| 296b | 8.4 | 497.0 |
| 297 | 38.0 | 607.0 |
| 298 | 4.7 | 166.0 |
| 299 | 9.0 | 239.0 |
| 300 | 36.0 | 513.0 |
| 301 | 12.0 | 404.0 |
| 302 | 21.0 | 686.0 |
| 303a | 2.6 | 48.0 |
| 303b | 2.0 | 28.0 |
| 304 | 29.0 | 251.0 |
| 305 | 6.5 | 58.0 |
| 306 | 2.8 | 27.0 |
| 307 | 2.5 | 18.0 |
| 308 | 2.2 | 26.0 |
| 309 | 1.4 | 6.1 |
| 310 | 81.0 | 2197.0 |
| 311 | 6.0 | 158.0 |
| 312 | 4.3 | 147.0 |
| 313 | 49.0 | 1139.0 |
| 314 | 1.6 | 44.0 |
| 315a | 16.0 | 591.0 |
| 315b | 4.8 | 207.0 |
| 316 | 1.1 | 12.0 |
| 317 | 0.7 | 18.0 |
| 318 | 257.0 | 1754.0 |
| 319 | 85.0 | 1746.0 |
| 320 | 134.0 | 1759.0 |
| 321a | 2.9 | 95.0 |
| 321b | 0.6 | 15.0 |
| 321c | 1.3 | 26.0 |
| 321d | 0.6 | 5.4 |
| 322a | 1.1 | 17.0 |
| 322b | 0.6 | 3.2 |
| 322c | 1.3 | 29.0 |
| 322d | 0.7 | 5.6 |
| 323a | 6.7 | 30.0 |
| 323b | 6.2 | 17.0 |
| 324 | 3.3 | 83 |
| 325a | 1.6 | 23 |
| 325b | 2.3 | 71 |
| 326 | 6.6 | 290 |
| 327a | 1.5 | 143 |
| 327b | 1.7 | 56 |
| 328a | 10 | 315 |
| 328b | 31 | 1972 |
| 329 | 2.7 | 175 |
| 331 | 3.9 | 229 |
| 332a | 1.9 | 367 |
| 332b | 3.5 | 160 |
| 333 | 10 | 402 |
| 334 | 84 | 899 |
| 335 | 5.1 | 351 |
| 336 | 97 | 1471 |
| 337 | 42 | 1732 |
| 338 | 13 | 544 |
| 339 | 3.1 | 187 |
| 340 | 13 | 499 |
| 341 | 4.7 | 77 |
| 342 | 4.9 | 61 |
| 343 | 3.2 | 43 |
| 344 | 6.4 | 626 |
| 345 | 64 | 1726 |
| 346 | 31 | 482 |
| 347 | 9.3 | 411 |
| 348 | 22 | 635 |
| 349a | 1.8 | 131 |
| 349b | 1.2 | 88 |
| 349c | 1.9 | 78 |
| 349d | 1.4 | 152 |
| 350 | 20 | 321 |
| 351 | 40 | 658 |
| 352a | 14 | 365 |
| 352b | 7 | 651 |
| 353 | 2.4 | 93 |
| 354 | 11 | 85 |
| 355 | 19 | 361 |
| 356 | 164 | 1580 |
| 357 | 23 | 817 |
| 358 | 2.6 | 221 |
| 360 | 12 | 358 |
| 361 | 8.4 | 349 |
| 362 | 3.8 | 133 |
| 364 | 1.6 | 102 |
| 365 | >10000 | >10000 |
| 366 | >10000 | >10000 |
| 367 | 1.7 | 117 |
| 368 | 1.2 | 95 |
| 369 | 0.7 | 13 |
| 370 | 1.1 | 9.7 |
| 371 | 3.5 | 265 |
| 372 | 6.8 | 395 |
| 373 | 45 | 1377 |
| 374 | 5.7 | 118 |
| 375 | 4.6 | 95 |
| 376 | 2.1 | 31 |
| 377 | 1.1 | 45 |
| 378 | 9.3 | 496 |
| 379 | 4.5 | 230 |
| 380a | 1.6 | 70 |
| 380b | 0.9 | 23 |
| 381a | 1.2 | 45 |
| 381b | 0.9 | 19 |
| 382 | 9.7 | 214 |
| 383a | 1.7 | 45 |
| 383b | 1.4 | 15 |
| 384a | 0.8 | 22 |
| 384b | 3.9 | 191 |
| 384c | 1.2 | 14 |
| 384d | 1.8 | 57 |
| 385 | 1.9 | 138 |
| 386 | 1 | 28 |
| 387a | 3 | 137 |
| 387b | 2 | 37 |
| 388a | 1.2 | 36 |
| 388b | 1.3 | 21 |
| 389 | 1.3 | 74 |
| 390 | 2.5 | 236 |
| 391a | 0.7 | 17 |
| 391b | 0.9 | 7.8 |
| 392 | 1.1 | 10 |
| 393 | 1.9 | 119 |
| 394 | 1.1 | 45 |
| 395a | 1.6 | 191 |
| 395b | 1.3 | 114 |
| 396 | 9.7 | 760 |
| 397 | 103 | 2016 |
| 398 | 4.9 | 53 |
| 399a | 6.2 | 264 |
| 399b | 3.8 | 90 |
| 400a | 6.9 | 154 |
| 400b | 3.4 | 64 |
| 401 | 2.1 | 105 |
| 402 | 5.1 | 101 |
| 403a | 1 | 10 |
| 403b | 1.1 | 7.3 |
| 404a | 18 | 717 |
| 404b | 14 | 458 |
| 405 | 10 | 644 |
| 406 | 601 | >10000 |
| 407 | 1.6 | 129 |
| 408 | 35 | 1100 |
| 409 | 5.8 | 210 |
| 410 | 27 | 884 |
| 411 | 9.4 | 386 |
| 412 | 1.8 | 147 |
| 413 | 3.5 | 412 |
| 414 | 7.3 | 232 |
| 415 | 26 | 700 |
| 416 | 2.1 | 67 |
| 417 | 3 | 18 |
| 418 | 2 | 32 |
| 419a | 2 | 298 |
| 419b | 1.8 | 78 |

### CDK2/Cyclin E1 and CDK1/Cyclin B1 Biochemical Assays

Compounds disclosed herein were tested for inhibition of CDK2/Cyclin E1 or CDK1/Cyclin B1 kinase in an assay based on the time-resolved fluorescence-resonance energy transfer (TR-FRET) methodology. The assay was carried out in 384-well low volume black plates in a reaction mixture containing CDK2/Cyclin E1 or CDK1/Cyclin B1, 1 mM ATP for CDK2/Cyclin E1 and CDK1/Cyclin B1, 0.15 μM Rb (Ser780)-biotin substrate and 0-10 μM compound in buffer containing 50 mM HEPES pH7.0, 0.02% NaN₃, 0.01% BSA, 0.1 mM Orthovanadate, 50 mM MgCl₂, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 90 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK2/Cyclin E1 or CDK1/Cyclin B1 kinase activity in presence of increasing concentrations of compounds was calculated based on the ratio of fluorescence at 665 nm to that at 620 nm. The IC50 for each compound was derived from fitting the data to the four-parameter logistic equation by Dotmatics. The IC50 data as measured for the Examples are shown in Table 2.

TABLE 2

| Example | CDK2/Cyclin E1 IC₅₀ (nM) | CDK1/Cyclin B1IC₅₀ (nM) |
|---|---|---|
| 254a | 12 | 3742 |
| 254b | 8.9 | 1375 |
| 330a | 84 | >10000 |
| 330b | 24 | 4093 |
| 359a | 14 | 8022 |
| 359b | 7.1 | 2894 |
| 363a | 2.2 | 2420 |
| 363b | 1.5 | 863 |
| 420 | 24 | 5508 |
| 421 | 135 | >10000 |
| 422a | 2.3 | 3246 |
| 422b | 1 | 980 |
| 423 | 2.7 | 252 |
| 424 | 84 | >10000 |
| 425 | 1954 | >10000 |
| 426 | 51 | 4869 |
| 427a | 1.1 | 145 |
| 427b | 0.8 | 68 |
| 428 | 21 | 4852 |
| 429 | 133 | >10000 |
| 430a | 3.6 | 1774 |
| 430b | 2.2 | 784 |
| 431 | 42 | >10000 |
| 432 | 23 | 1316 |
| 433a | 18 | 3095 |
| 433b | 18 | 1448 |
| 434 | 1.7 | 747 |
| 435 | 1.3 | 423 |
| 436 | 1.5 | 122 |
| 437 | 249 | >10000 |
| 438 | 4.7 | 583 |
| 439 | 2.7 | 215 |
| 440 | 189 | >10000 |
| 441 | 47 | 5058 |
| 442 | 55 | 2986 |
| 443 | 1.6 | 582 |
| 444 | 16 | 7870 |
| 445 | 1.8 | 1053 |
| 446 | 1.7 | 1401 |
| 447 | 4.8 | 1277 |
| 448 | 1.1 | 65 |
| 449 | 4.1 | 1191 |
| 450 | 7.6 | 6178 |
| 451 | 9.9 | 7055 |
| 452 | 0.5 | 487 |
| 453 | 0.9 | 446 |
| 454 | 0.5 | 114 |
| 455 | 0.9 | 97 |
| 456 | 0.5 | 550 |
| 457 | 1.8 | 1114 |
| 458 | 12 | 3497 |
| 459 | 6 | 1973 |
| 460 | 3.1 | 593 |
| 461 | 0.3 | 271 |
| 462 | 0.7 | 349 |
| 463 | 0.4 | 140 |
| 464 | 0.8 | 325 |
| 465 | 18 | 4890 |
| 466 | 63 | >10000 |
| 467 | 530 | >10000 |

1. A compound of Formula (I): 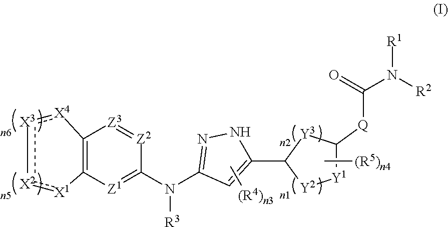
or a N-oxide thereof, or a pharmaceutically acceptable salt thereof, or a stereoisomer thereof, or a tautomer, or a deuterated analog thereof, or a prodrug thereof, wherein:
n1 is 0, 1, or 2;
n2 is 1, 2 or 3;
n3 is 0, 1, or 2;
n4 is 0, 1, 2, 3 or 4;
n5 and n6 are each independently 0 or 1, provided that n5 and n6 are not 0 at the same time;
each of [symbol] is independently a single bond or double bond, provided that two double bonds are not connected directly;
X¹, X², X³ and X⁴ are each independently selected from N, C, S(═O)₂ or S(═O), wherein each Nis independently substituted with 0 or 1 R^{Xa} groups as allowed by valence, and each C is independently substituted with one or two R^{Ab} groups as allowed by valence;
R^{Xa} is each independently selected from hydrogen, —C₁-C₈alkyl, —C₂-C₈alkenyl, —C₂-C₈alkynyl, —C₃-C₈cycloalkyl, 3- to 12-membered heterocyclyl, —C₆-C₁₂aryl or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{Xaa};
R^{Xaa} is each independently selected from hydrogen, deuterium, halogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl, 5- to 12-membered heteroaryl, oxo (═O), —OR^{Xab}, —CN, —SO₂R^{Xab}, —SO₂NR^{Xab}R^{Xac}, —C(O)R^{Xab}, —CO₂R^{Xab}, —C(O)NR^{Xab}R^{Xac}, —NR^{Xab}R^{Xac}, —NR^{Xab}COR^{Xac}, —NR^{Xab}CO₂R^{Xac} or —NR^{Xab}SO₂R^{Xac}, wherein each of said —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{Xad}, —SO₂R^{Xad}, —SO₂NR^{Xad}R^{Xac}, —C(O)R^{Xad}, —CO₂R^{Xad}, —C(O)NR^{Xad}R^{Xas}, —NR^{Xad}R^{Xac}, —NR^{Xad}COR^{Xae}, —NR^{Xad}CO₂R^{Xae} or —NR^{Xad}SO₂R^{Xae};
R^{Xab}, R^{Xac}, R^{Xad} and R^{Xae} are each independently selected from hydrogen, deuterium, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with halogen, —C₁-C₈alkyl, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —CN, —OH, or —C₁-C₈alkoxyl;
R^{Xb} is each independently selected from hydrogen, halogen, —C₁-C₈alkyl, C₃-C₈cycloalkyl, 3- to 8-membered heterocyclyl, C₆-C₁₂aryl, 5- to 12-membered heteroaryl, oxo (═O), —NR^{Xba}R^{Xbb}, —OR^{Xba}, —SR^{Xba}, —SO₂R^{Xba}, —SO₂NR^{Xba}R^{Xbb}, —C(O)R^{Xba}, —CO₂R^{Xba}, —C(O)NR^{Xba}R^{Xbb}, —NR^{Xba}COR^{Xbb}, —NR^{Xba}CO₂R^{Xbb} or —NR^{Xba}SO₂R^{Xbb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Xbe}, or
two RAD together with the atom(s) to which they are attached, form a 3- to 12-membered ring, said ring comprising 0-3 heteroatoms independently selected from nitrogen, oxygen or optionally oxidized sulfur as ring member(s); said ring is optionally substituted with at least one substituent R^{Xbc}, R^{Xba} and R^{Xbb} are each independently selected from hydrogen, deuterium, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C3-C₈cycloalkyl, 3- to 8-membered heterocyclyl, C₆-C₁₂aryl, or 5- to 12-membered heteroaryl is optionally substituted with at least one substituent R^{Xbd}, R^{Xbc} and R^{Xbd} are each independently selected from hydrogen, deuterium, halogen, hydroxy, -C1-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Xbe}R^{Xbf}, —OR^{Xbe}, —SR^{Xbe}, —SO₂R^{Xbe}, —SO₂NR^{Xbe}R^{Xbf}, —C(O)R^{Xbe}, —CO₂R^{Xbe}, —C(O)NR^{Xbe}R^{Xbf}, —NR^{Xbe}COR^{Xbf}, —NR^{Xbe}COO₂R^{Xbf} or —NR^{Xbe}SO₂R^{Xbf} or —CN;
R^{Xbe} and R^{Xbf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
Y¹, Y² and Y³ are each independently selected from O, C or N, wherein C and N are independently substituted with 1 or 2 R⁵ groups or hydrogen atoms as allowed by valence;
Q is selected from O or NR^{Q};
R¹, R², R³ and R° are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{1a}; or
R¹ and R² together with the atom to which they are attached, form a 3- to 12-membered ring, said ring comprising 0-3 additional heteroatoms independently selected from nitrogen, oxygen or optionally oxidized sulfur as ring member(s); said ring is optionally substituted with at least one substituent R^{1b}b;
R^{1a} and R^{1b} are each independently selected from hydrogen, halogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl, 5- to 12-membered heteroaryl, oxo (═O), —OR^{1c}, —CN, —SO₂R^{1c}, —SO₂NR^{1c}R^{1d}, —C(O)R^{1c}, —CO₂R^{1c}, —C(O)NR^{1c}R^{1d}, —NR^{1c}R^{1d}, —NR^{1c}COR^{1d}, —NR^{1c}COR₂^{1d} or —NR^{1c}SO₂R^{1d}, wherein each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{1e}, —SO₂R^{1e}, —SO₂NR^{1e}R^{1f}, —C(O)R^{1e}, —CO₂R^{1f}, —C(O)NR^{1e}R^{1f}, —NR^{1e}R^{1f}, —NR^{1e}COR^{1f}, —NR^{1e}CO₂R^{1f} or —NR^{1e}SO₂R^{1f};
R^{1e}, R^{1d}, R^{1e} and R^{1f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁴ and R⁵ are each independently selected from hydrogen, halogen, —C₁-C₈alkyl, —C₂-C₈alkenyl, —C₂-C₈alkynyl, —C₁-C₈alkoxy, or —C₃-C₈cycloalkyl;
z¹, Z² and Z³ are each independently selected from —CR^{Z}, or N;
R², at each occurrence, is independently selected from hydrogen, halogen, —C₁-C₈alkyl, C₃-C₈cycloalkyl, 3- to 8-membered heterocyclyl, C₆-C₁₂aryl, 5- to 12-membered heteroaryl, —NR^{Za}R^{Zb}, —OR^{Za}, —SR^{Za}, —SO₂R^{Za}, —SO₂NR^{Za}R^{2b}, C(O)R^{Za}, —CO₂R^{Za}, —C(O)NR^{Za}R^{2b}, —NR^{Za}COR^{Zb}, —NR^{Za}CO₂R^{Zb} or —NR^{Za}SO₂R^{Zb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc};
R^{Za} and R^{2b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Zd},
R^{Zc} and R^{Zd} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Ze}R^{Zf}, —OR^{Ze}, —SR^{Ze}, —SO₂R^{Ze}, —SO₂NR^{Ze}R^{Zf}, —C(O)R^{Ze}, —CO₂R^{Ze}, —C(O)NR^{Ze}R^{Zf}, —NR^{Ze}COR^{Zf}, —NR^{Ze}CO₂R^{Zf} or —NR^{Ze}SO₂R^{Zf} or —CN;
R^{ze} and R^{Zf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl.

2. The compound of claim 1, wherein the compound is selected from (IIa), (IIb), (IIc), (IId), (IIe), (III), (IIg), (IIh), (Ii), (II), (Illa), (IIb), (IIc), (IIId), (Ille), (IIIf), (IIIg), (IIIh), (IVa), (IVb), (IVc), (IVd), (IVe), (IVf), (IVg), (IVh), (IVi), (IVi), (IVk), (IVl), (IVm), (IVn), (IVo), (IVp), (IVg), (IVr), (IVs), (IVt), (IVu), (IVv), (IVw), (IVx), (Va), (VIa), (Vb), (Vc), (VIb) and (VIc), (Vd), (VId), (Ve), (Vf), (VIe) and (VIf):

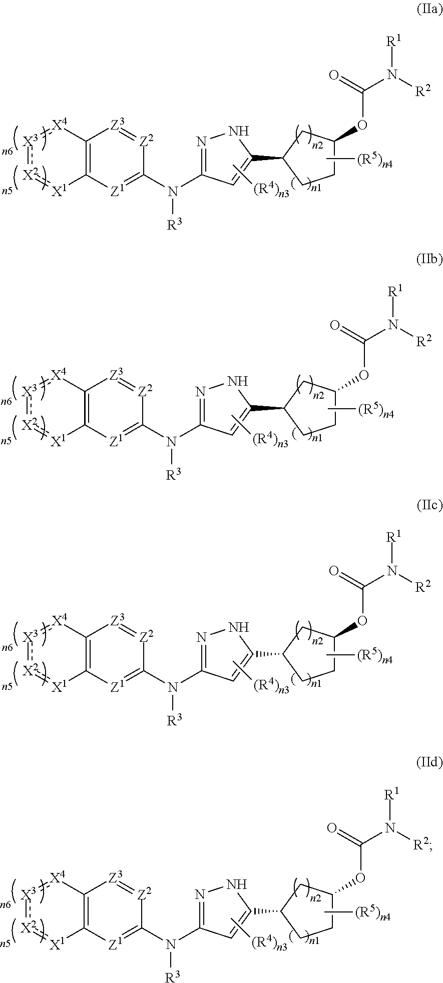

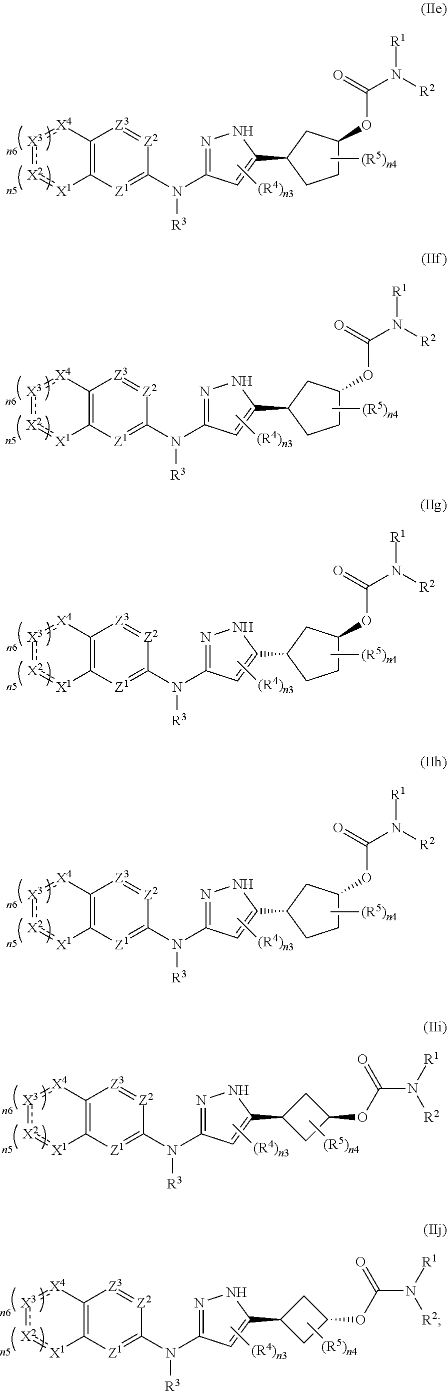

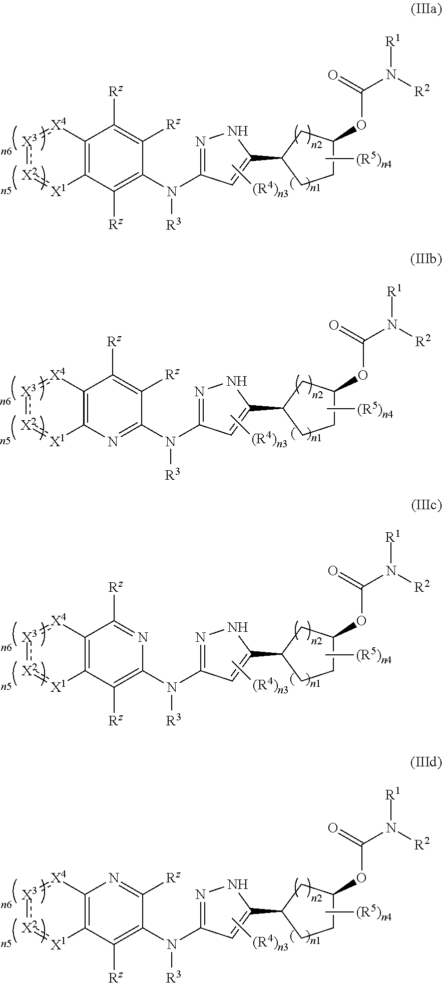

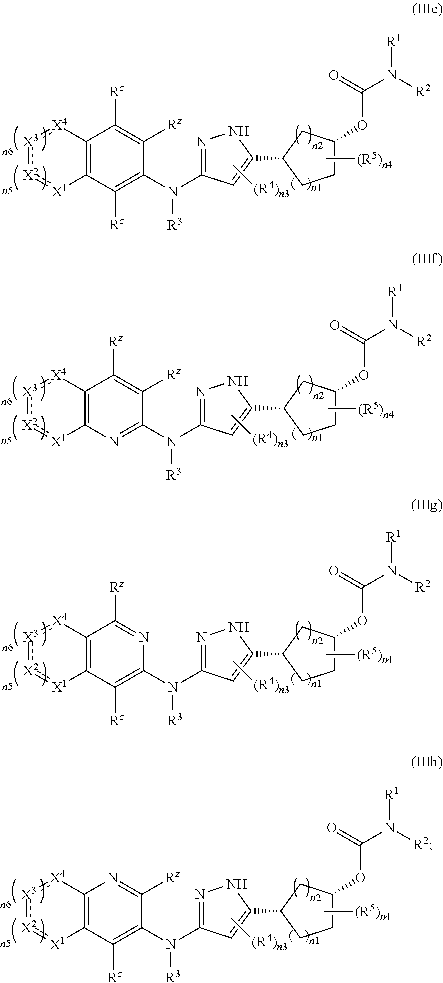

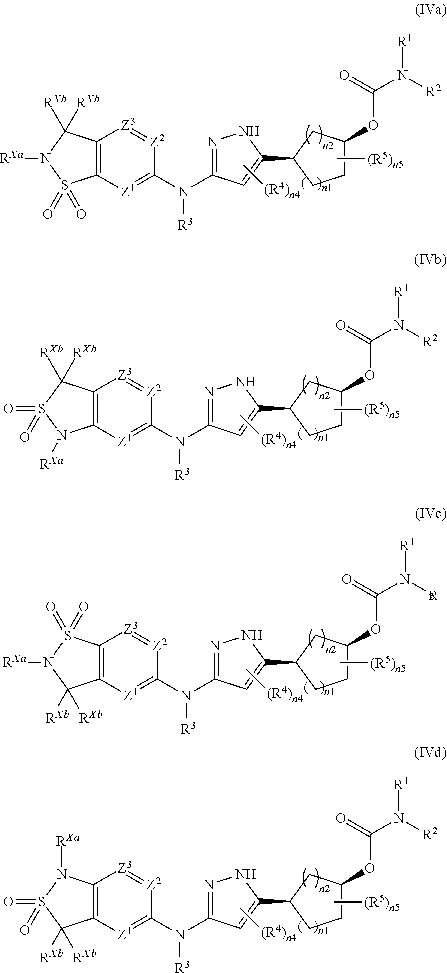

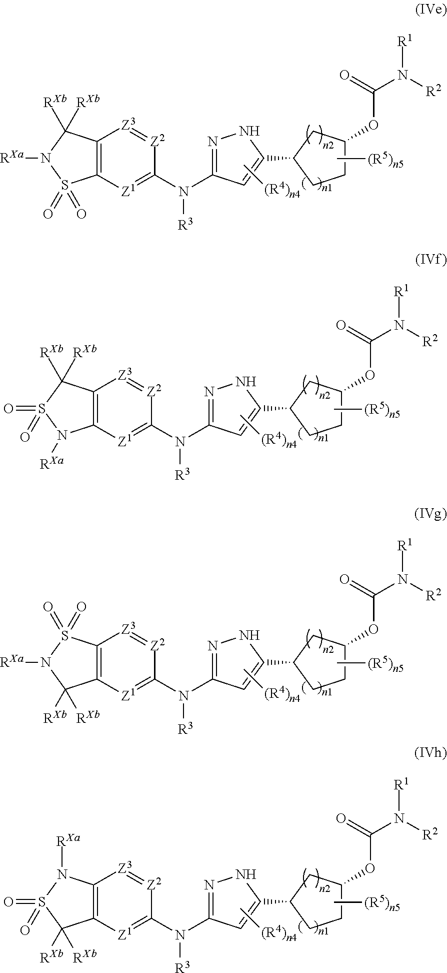

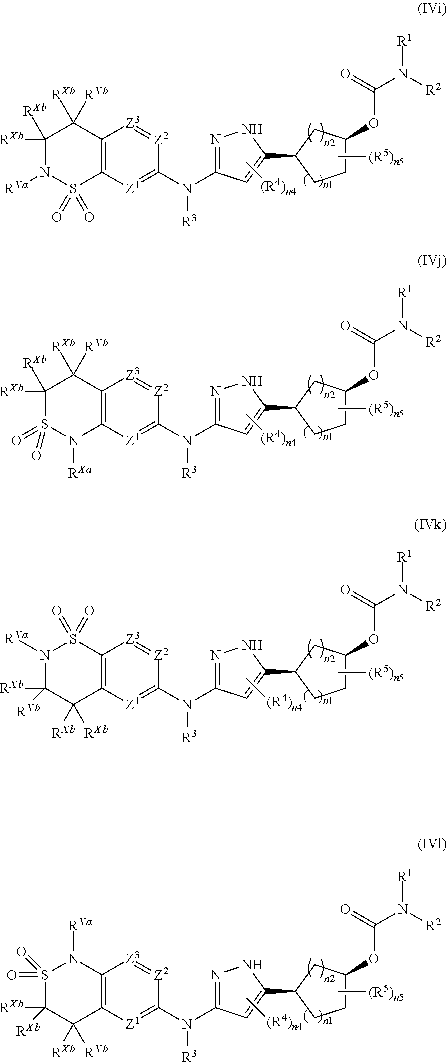

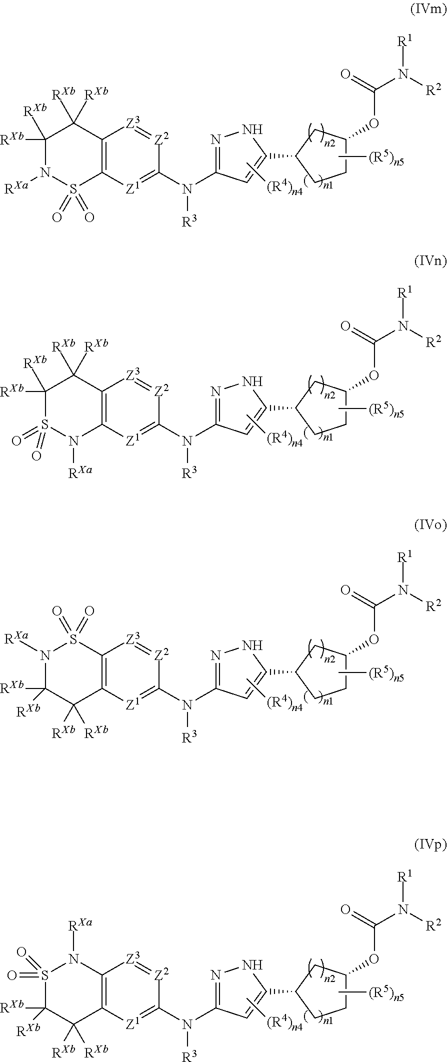

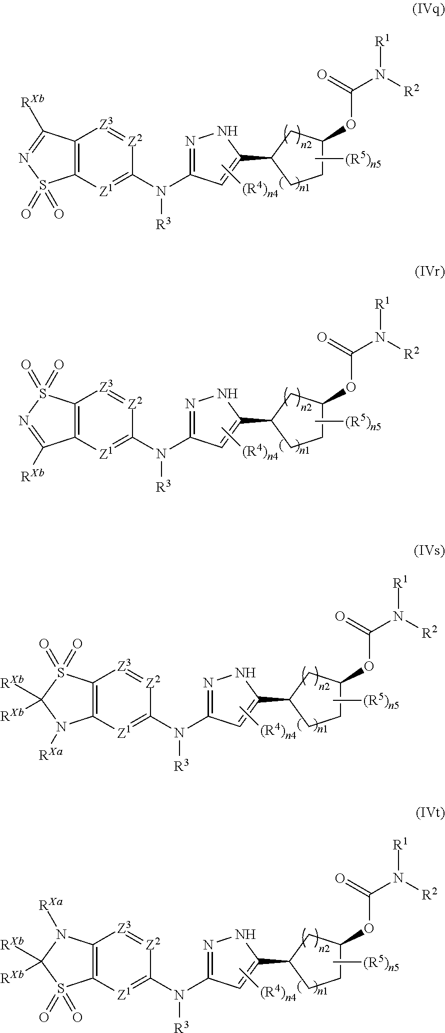

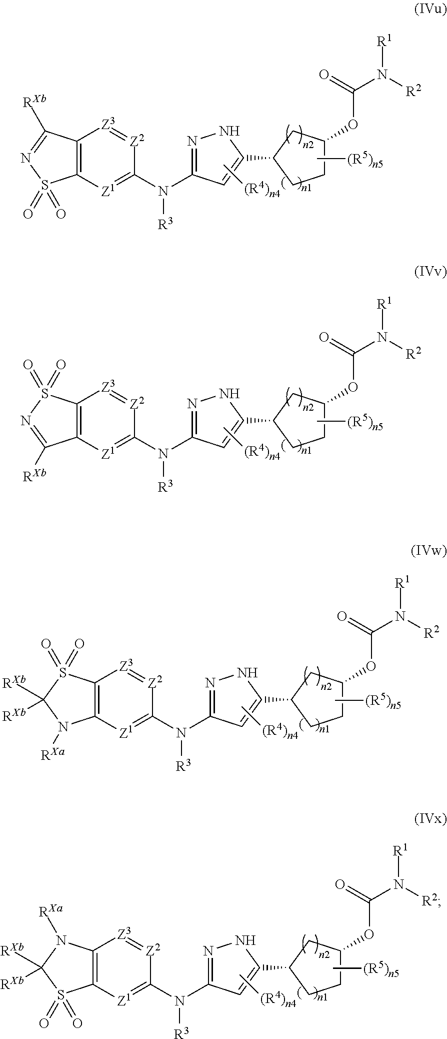

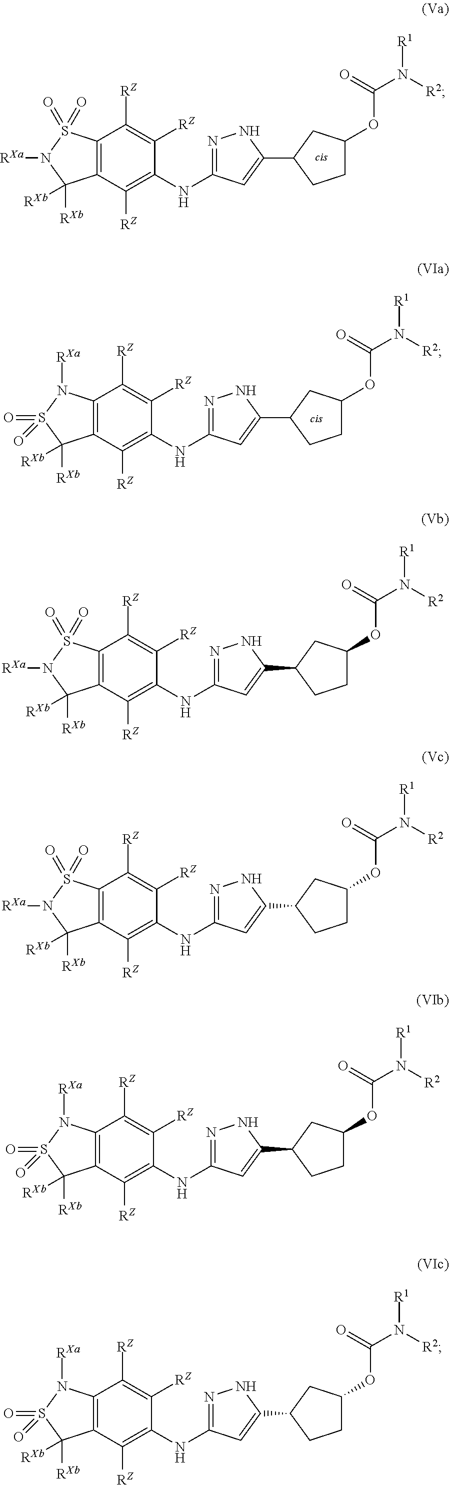

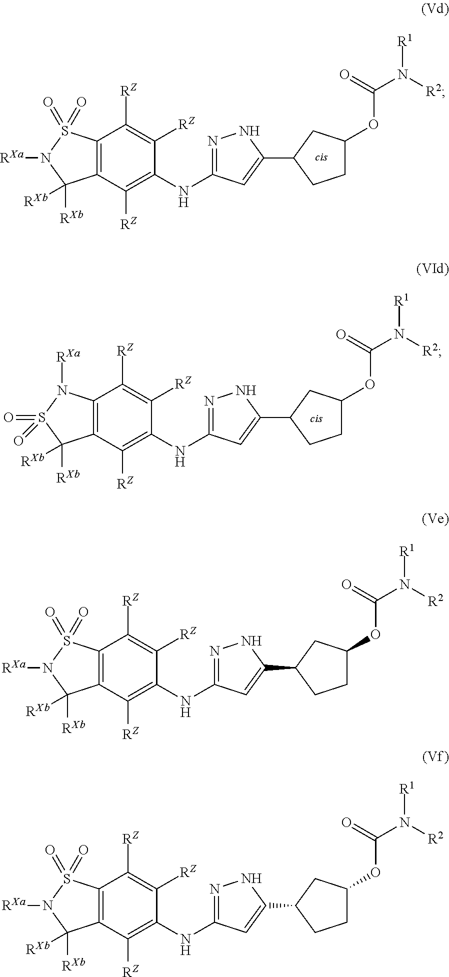

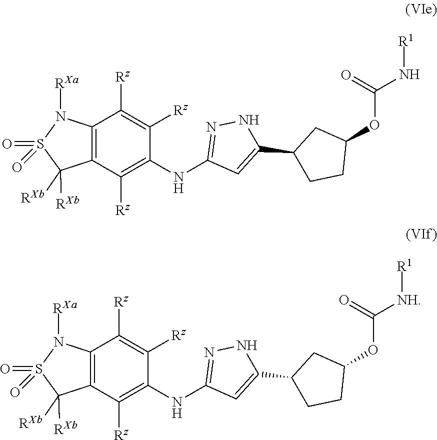

3. (canceled)

4. (canceled)

5. The compound of claim 1, wherein R¹, R², R³ and R^{Q} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, cyclononyl, cyclodecyl, 3- to 8-membered heterocyclyl, phenyl or 5- to 12-membered heteroaryl, wherein each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, cyclononyl, cyclodecyl, 3- to 8-membered heterocyclyl, phenyl or 5- to 12-membered heteroaryl is optionally substituted with at least one substituent R^{1a}; or
R¹ and R² together with the atom(s) to which they are attached, form a 4-5- or 6-membered ring;
said ring is optionally substituted with at least one substituent R^{1b};
R^{1a} and R^{1b} are each independently selected from hydrogen, —F, —Cl, —Br, —I, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{1c}, —CN, —SO₂R^{1c}, —SO₂NR^{1c}R^{1d}, —C(O)R^{1c}, —CO₂R^{1c}, —C(O)NR^{1c}CR^{1d}, —NR^{1c}OR^{1d}, —NR^{1c}COR^{1d}, —NR^{1c}CO₂R^{1d} or —NR^{1c}SO₂R^{1d}, wherein each of said methyl, ethyl, propyl, butyl, pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{1e}, —SO₂R^{1e}, —SO₂NR^{1e}R^{1f}, —C(O)R^{1e}, —COR^{1f}, —C(O)NR^{1e}R^{1f};
NR^{1e}R^{1f}, —NR^{1e}COR^{1f}, —NR^{1e}CO₂R^{1f} or —NR^{1e}SO₂R^{1f}, R^{1e}, R^{1d}, R^{1e} and R^{1f} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl-(n-propyl or isopropyl), butyl-(n-butyl, sec-butyl, iso butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy.

6. The compound of claim 1, wherein R¹ is independently selected from hydrogen, methyl, ethyl, propyl, butyl, pentyl, hexyl, cyclopropyl, cyclobutyl, cyclopentyl, cyclohexyl, azetidinyl, oxetanyl, oxanyl, bicyclo[1.1.1]pentanyl, spiro[3.3]heptanyl, spiro[2.3]hexanyl or tetrahydrofuranyl, wherein each of said methyl, ethyl, propyl, butyl, pentyl, hexyl, cyclopropyl, cyclobutyl, cyclopentyl, cyclohexyl, azetidinyl, oxetanyl, oxanyl, bicyclo[1.1.1]pentanyl, spiro[3.3]heptanyl, spiro[2.3]hexanyl or tetrahydrofuranyl is optionally substituted with at least one substituent R^{1a};
R^{1a} is independently selected from hydrogen, —F, —CI, —Br, —I, oxo (═O), methyl, ethyl, propyl, cyclopropyl, cyclobutyl, cyclopentyl, cyclohexyl, azetidinyl, oxetanyl, bicyclo[1.1.1]pentanyl, spiro[3.3]heptanyl, spiro[2.3]hexanyl, tetrahydrofuranyl or —OR^{1c};
wherein each of said methyl, ethyl, propyl, cyclopropyl, cyclobutyl, cyclopentyl, cyclohexyl is optionally substituted with OR^{1c};
R^{1c} is independently selected from hydrogen, methyl, ethyl, propyl, butyl, cyclopropyl, cyclobutyl; and R² is hydrogen; or
R¹ and R² together with the nitrogen atom to which they are attached, form a 4- or 5-membered ring; said ring is optionally substituted with at least one substituent R^{1b};
R^{1b} is independently selected from hydrogen, —F, —Cl, methyl, ethyl, propyl, butyl or —OH.

7. (canceled)

8. The compound of claim 1, wherein the 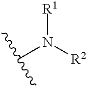
moiety is selected from

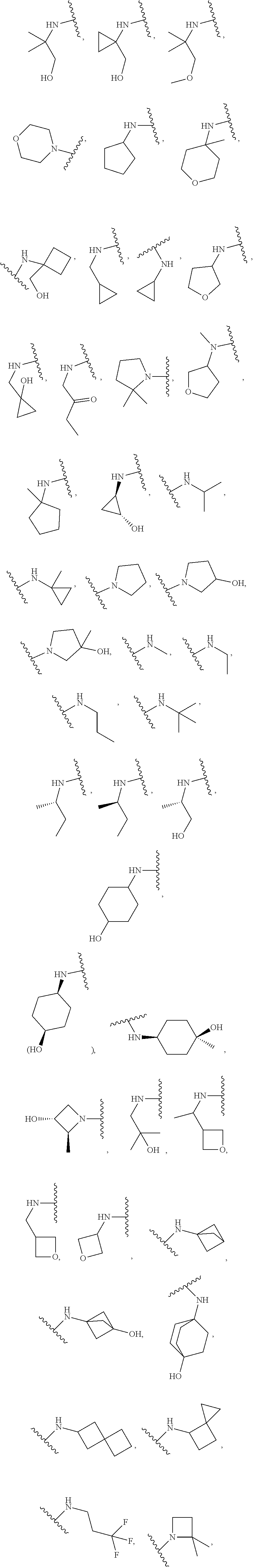

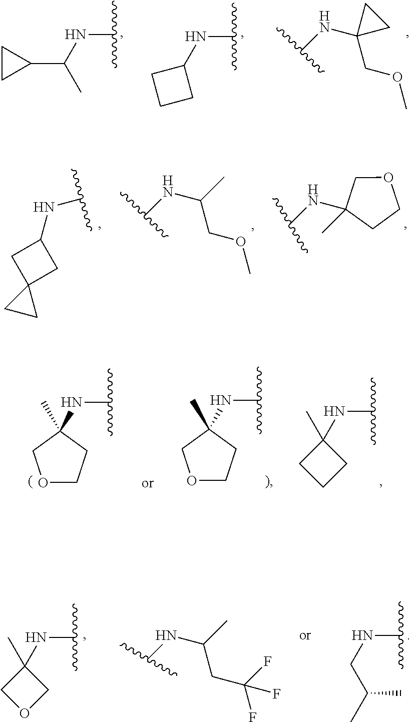

9. The compound of claim 1, wherein R⁴ and R⁵ are each independently selected from hydrogen, —F, —Cl, —Br, —I, methyl, ethyl, propyl, butyl, pentyl, hexyl, heptyl, octyl, —C₂-C₂alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl.

10. The compound of claim 1, wherein the 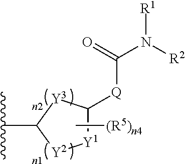
moiety is selected from

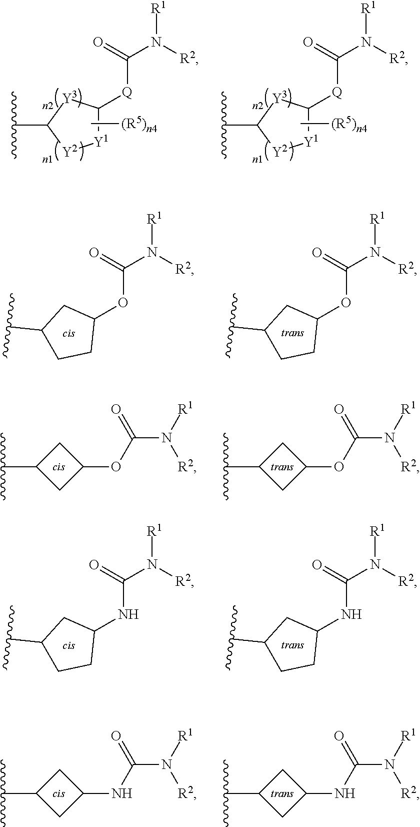

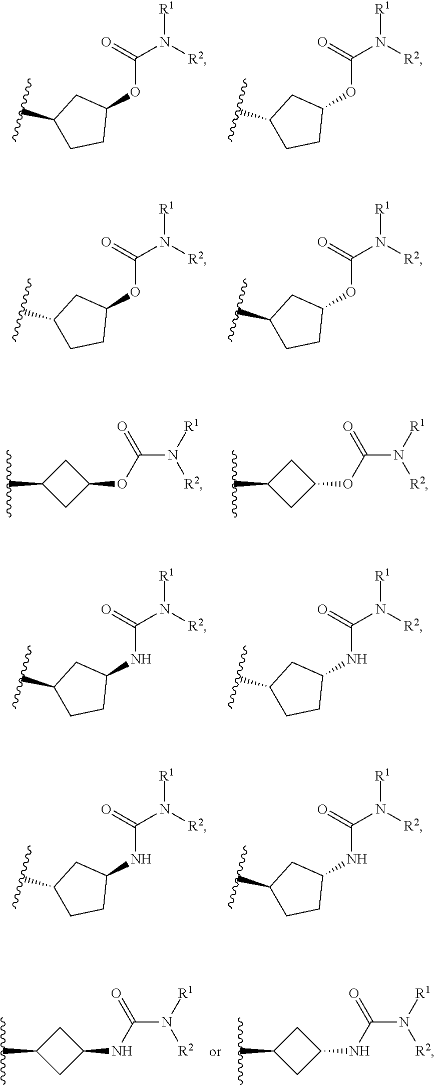

11. The compound of claim 1, wherein at most two of Z¹, Z² and Z³ are N.

12. The compound of claim 1, wherein
R^{Z}, at each occurrence, is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NR^{Za}R^{Zb}, —OR^{Za}, —SR^{Za}, or —CN;
R^{Za} and R^{2b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

13. The compound of claim 1, wherein the 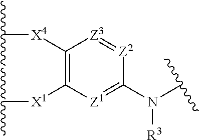
moiety is selected from

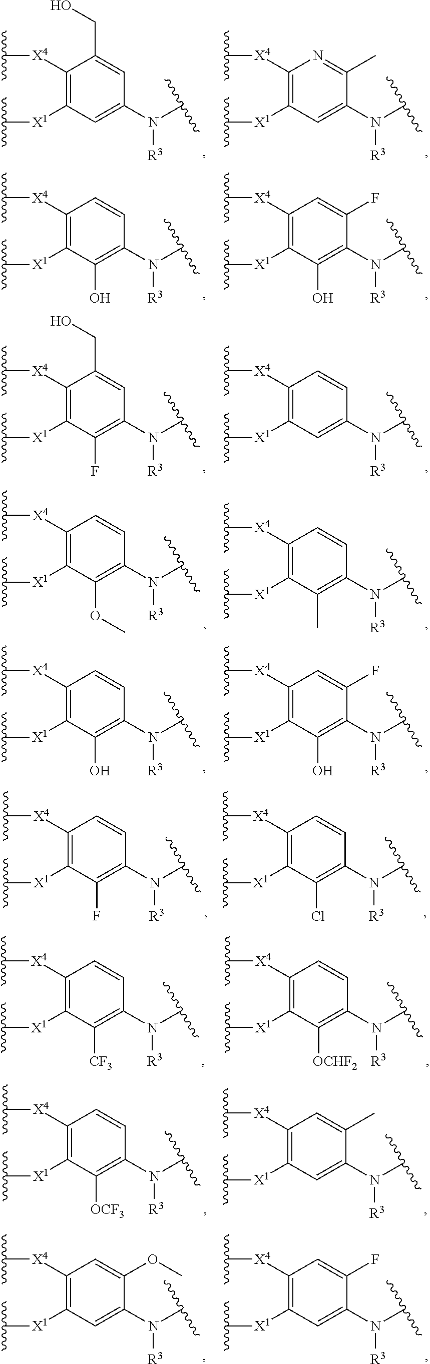

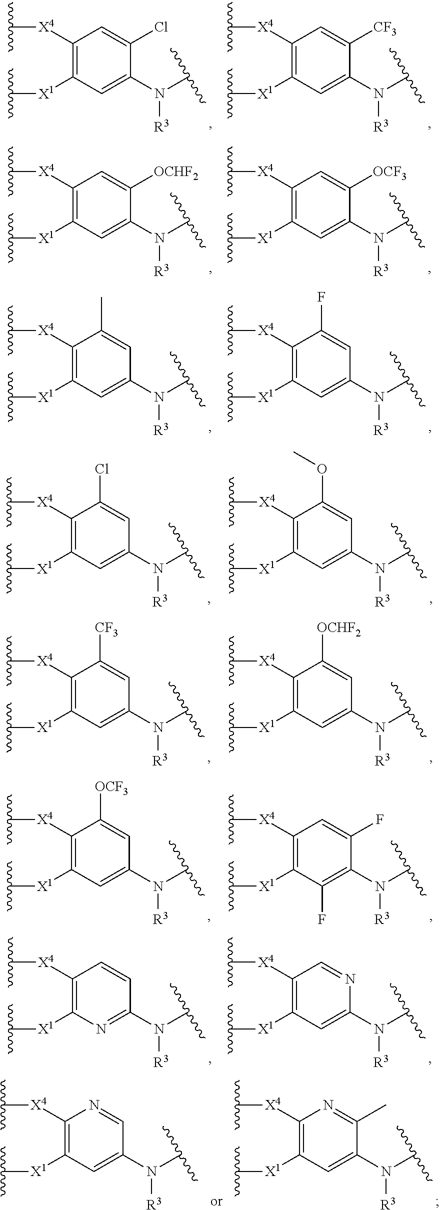

14. The compound of claim 1, wherein at most one of X¹, X², X³ and X⁴ is selected from N or NR^{Xa}, at most one of X¹, X², X³ and X⁴ is selected from S(═O)₂ or S(═O), provided that X¹, X², X³ and X⁴ are allowed by valence;
R^{Xa} is each independently selected from hydrogen, methyl, ethyl, propyl, butyl, pentyl, hexyl heptyl, octyl, cyclopropyl, cyclobutyl, cyclopentyl, cyclohexyl, cycloheptyl, cyclooctyl or 3- to 8-membered heterocyclyl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one substituent R^{Xaa};
R^{Xaa} is independently selected from hydrogen, —F, —Cl, —Br, —I, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{Xab}, —CN, —C(O)R^{Xab} or —NR^{Xab}R^{Xac}, wherein each of said methyl, ethyl propyl, butyl, pentyl, hexyl, heptyl, octyl, —C₂-C₈alkenyl, —C₂-C₈alkynyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{Xad}, SO₂R^{Xad}, C(O)R^{Xad} or —NR^{Xad}R^{Xae};
R^{Xab}, R^{Xac}, R^{Xad} and R^{Xae} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, hepthoxy, or octoxy;
R^{Xb} is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, -CHEF, —CHF₂, —CF₃, -CH₂CH₂F, —CH₂CHF₂, —CH₂CF₃, oxo (═O), —NR^{Xba}R^{Xbb}, —OR^{Xba}, SR^{Xba}, SO₂R^{Xba}, SO₂NR^{Xba}R^{Xbb}, C(O)R^{Xba}, —CO₂R^{Xba}, —C(O)NR^{Xba}R^{Xbb}, —NR^{Xba}COR^{Xbb}—NR^{Xba}COR^{Xbb} NR^{Xba}SO₂R^{Xbb} or —CN; or
two R^{Xb} together with the atom(s) to which they are attached, form a 3-, 4-, 5- or 6-membered ring;
said ring is optionally substituted with at least one substituent R^{Xbc};
R^{Xba} and R^{Xbb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{Xbe} is each independently selected from —F, —Cl, —Br, —I, hydroxy, methyl, ethyl, propyl, butyl, methoxy, ethoxy, propoxy, butoxy, cyclopropyl, cyclobutyl, cyclopentyl, cyclohexyl, oxo (═O) or —CN.

15. The compound of claim 1, wherein
R^{Xa} is each independently selected from hydrogen, methyl, ethyl, propyl, butyl, pentyl, cyclopropyl, cyclobutyl, cyclopentyl, cyclohexyl, piperidinyl, tetrahydrofuranyl, azetidinyl or pyrrolidinyl, wherein each of said methyl, ethyl, propyl, butyl, pentyl, cyclopropyl, cyclobutyl, cyclopentyl, cyclohexyl, piperidinyl, tetrahydrofuranyl, azetidinyl, oxetanyl or pyrrolidinyl is optionally substituted with at least one substituent R^{Xaa};
R^{Xaa} is independently selected from hydrogen, —F, —Cl, —Br, methyl, ethyl, propyl, butyl, cyclopropyl, cyclobutyl, cyclopentyl, cyclohexyl, oxo (═O), —OH, methoxy, ethoxy, propoxy, butoxy, pentoxy, hexoxy, —CN, benzyl, —NMe₂, —NH₂, —SO₂Me, —COCH₃, 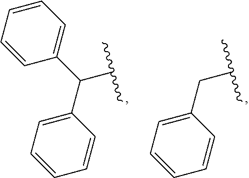 [SMILES: *C(c1ccccc1)c1ccccc1 ; *Cc1ccccc1]
oxetanyl, azetidinyl or 1-methylazetidinyl; wherein each of said methyl, ethyl, propyl, butyl, cyclopropyl, cyclobutyl, cyclopentyl, cyclohexyl, oxetanyl, or azetidinyl is optionally substituted with F, —C, methyl, ethyl, propyl, butyl, pentyl, hexyl, heptyl, —OR^{Xad}, —SO₂R^{Xad}, —C(O)R^{Xad};
R^{Xad} is selected from hydrogen, methyl, and ethyl;
R^{Xb} is independently selected from hydrogen, —F, —Cl, —Br, —I, methyl, -CD₃, ethyl, propyl, butyl, pentyl, hexyl, heptyl, octyl, cyclopropyl, cyclobutyl, cyclopentyl, cyclohexyl, cycloheptyl cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NH₂, —OH, methoxy, ethoxy, propoxy, butoxy, pentoxy, hexoxy, hepthoxy, octoxy or —CN; or
two R^{Xb} together with the atom(s) to which they are attached, form a 3-4-5- or 6-membered ring.

16. (canceled)

17. (canceled)

18. The compound of claim 1, wherein the 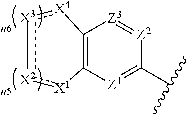
moiety is selected from

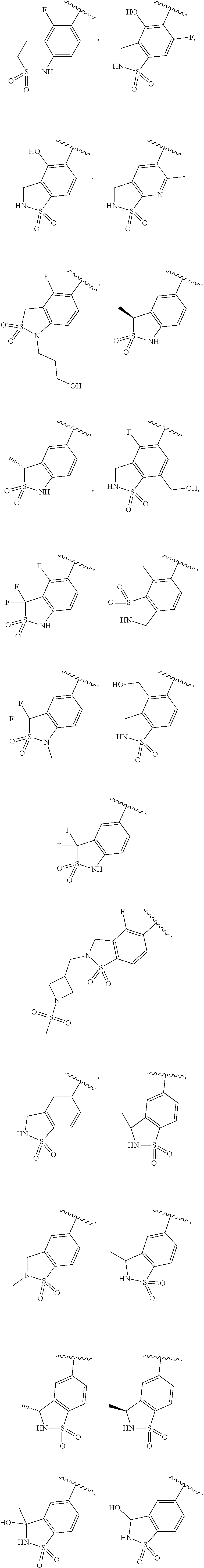

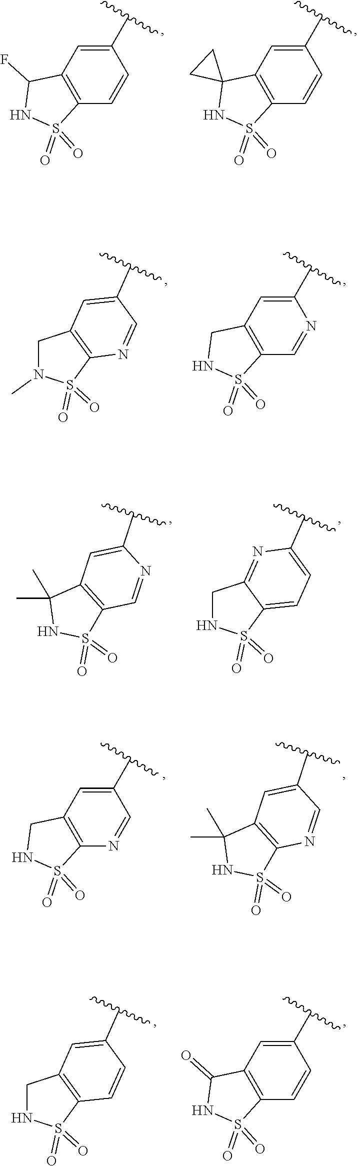

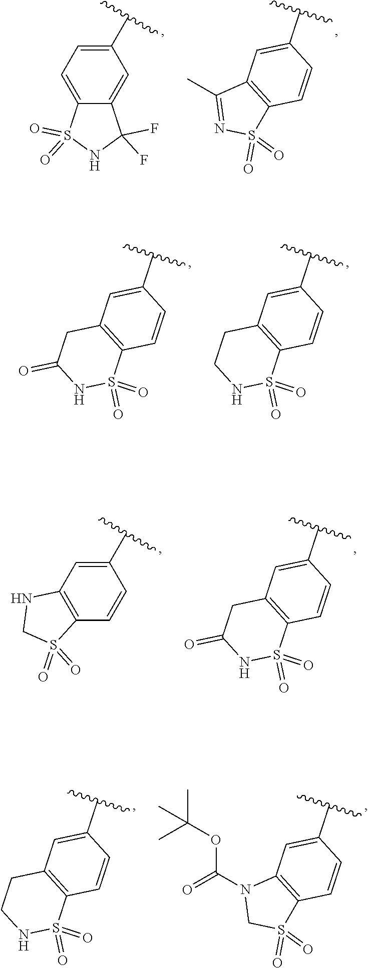

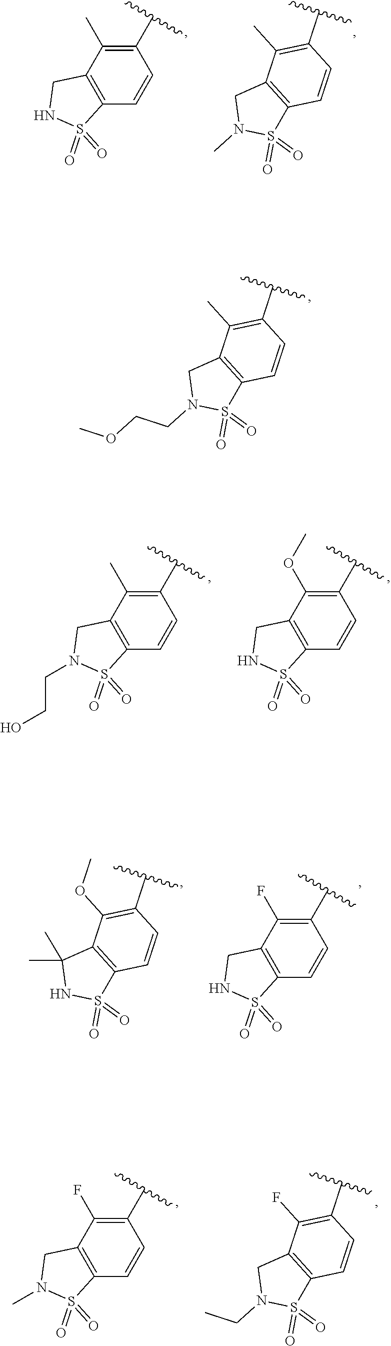

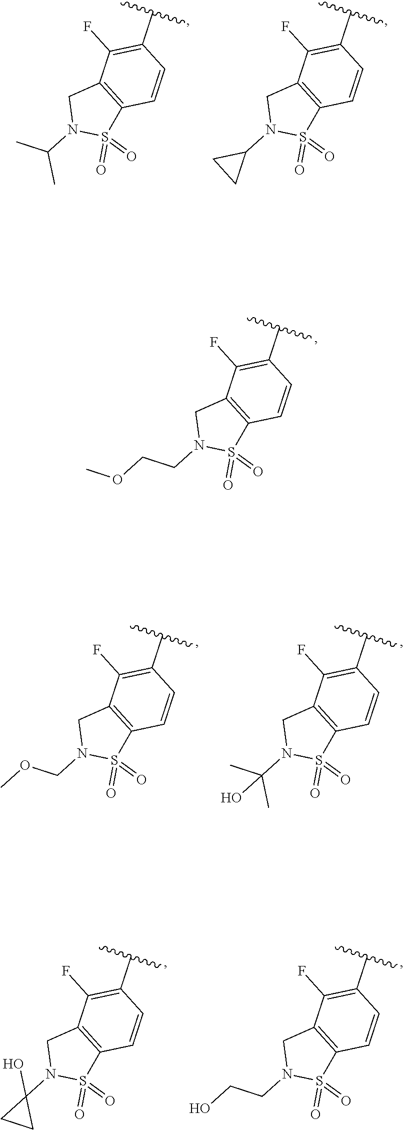

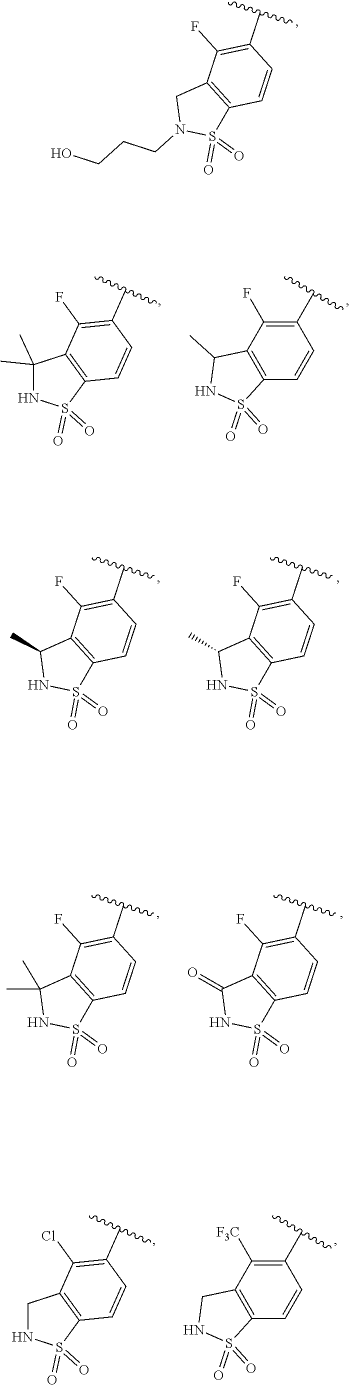

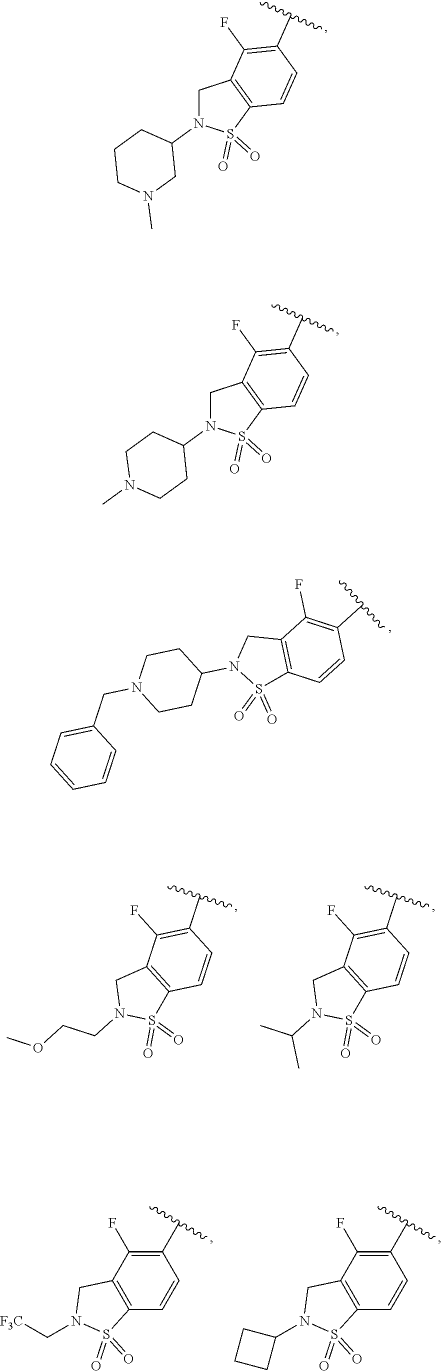

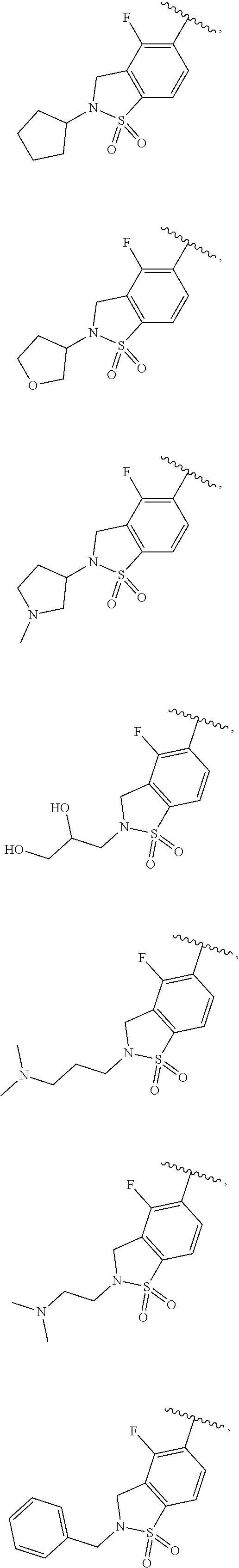

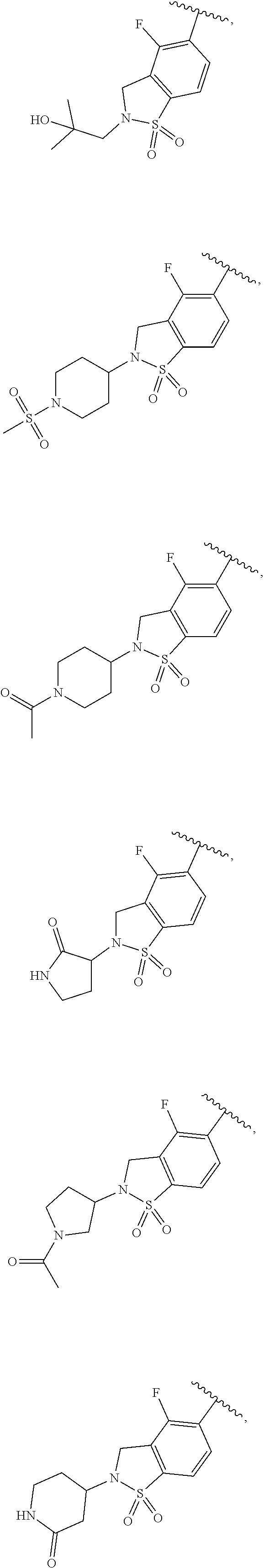 [SMILES: *c1ccc2c(c1F)CN(CC(C)(C)O)S2(=O)=O ; *c1ccc2c(c1F)CN(C1CCN(S(C)(=O)=O)CC1)S2(=O)=O ; *c1ccc2c(c1F)CN(C1CCN(C(C)=O)CC1)S2(=O)=O ; *c1ccc2c(c1F)CN(C1CCNC1=O)S2(=O)=O ; *c1ccc2c(c1F)CN(C1CCN(C(C)=O)C1)S2(=O)=O ; *c1ccc2c(c1F)CN(C1CCNC(=O)C1)S2(=O)=O]

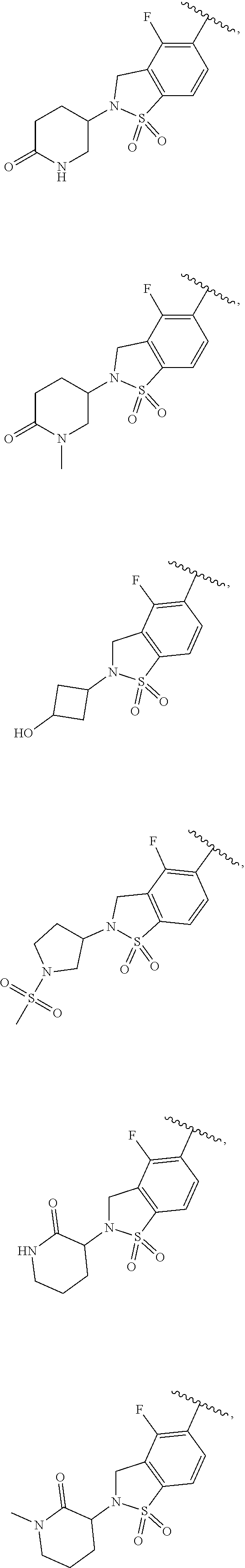 [SMILES: *c1ccc2c(c1F)CN(C1CCC(=O)NC1)S2(=O)=O ; *c1ccc2c(c1F)CN(C1CCC(=O)N(C)C1)S2(=O)=O ; *c1ccc2c(c1F)CN(C1CC(O)C1)S2(=O)=O ; *c1ccc2c(c1F)CN(C1CCN(S(C)(=O)=O)C1)S2(=O)=O ; *c1ccc2c(c1F)CN(C1CCCNC1=O)S2(=O)=O ; *c1ccc2c(c1F)CN(C1CCCN(C)C1=O)S2(=O)=O]

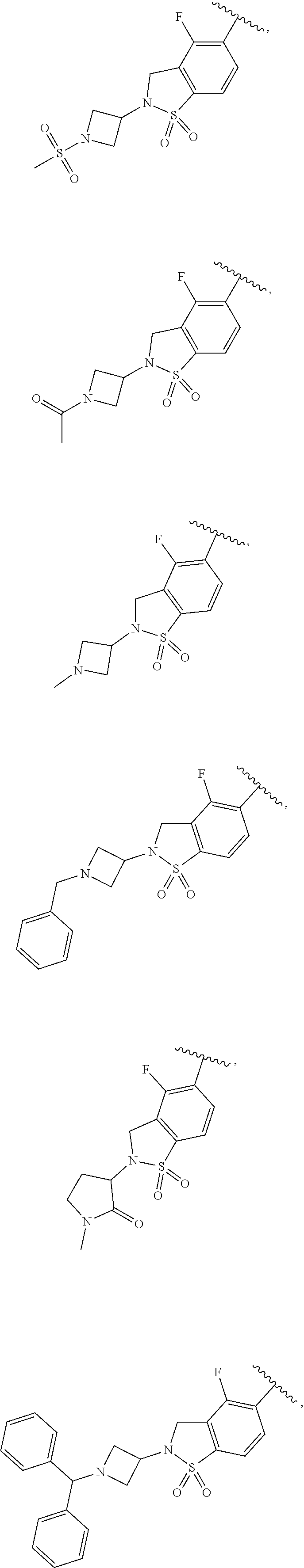 [SMILES: *c1ccc2c(c1F)CN(C1CN(S(C)(=O)=O)C1)S2(=O)=O ; *c1ccc2c(c1F)CN(C1CN(C(C)=O)C1)S2(=O)=O ; *c1ccc2c(c1F)CN(C1CN(C)C1)S2(=O)=O ; *c1ccc2c(c1F)CN(C1CN(Cc3ccccc3)C1)S2(=O)=O ; *c1ccc2c(c1F)CN(C1CCN(C)C1=O)S2(=O)=O ; *c1ccc2c(c1F)CN(C1CN(C(c3ccccc3)c3ccccc3)C1)S2(=O)=O]

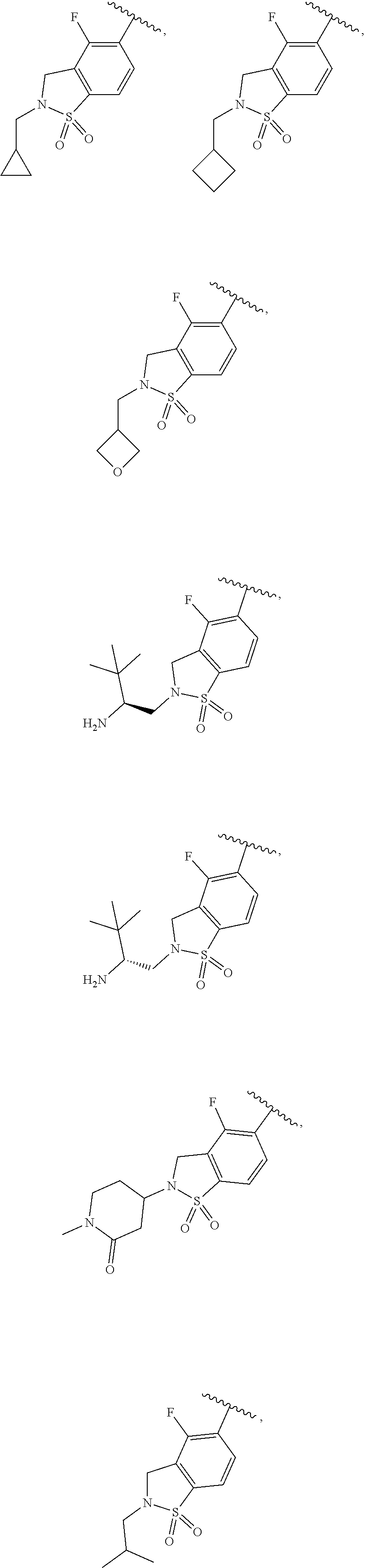

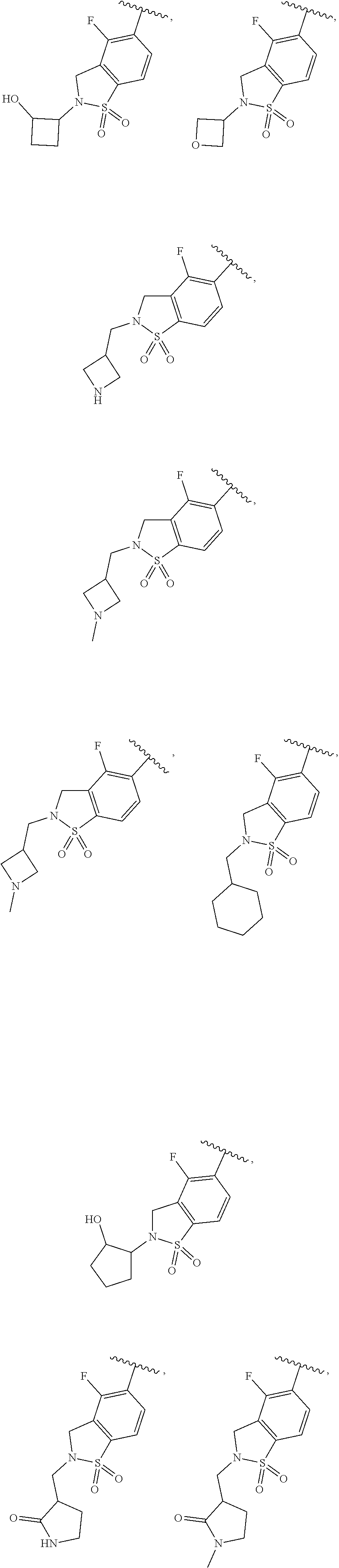

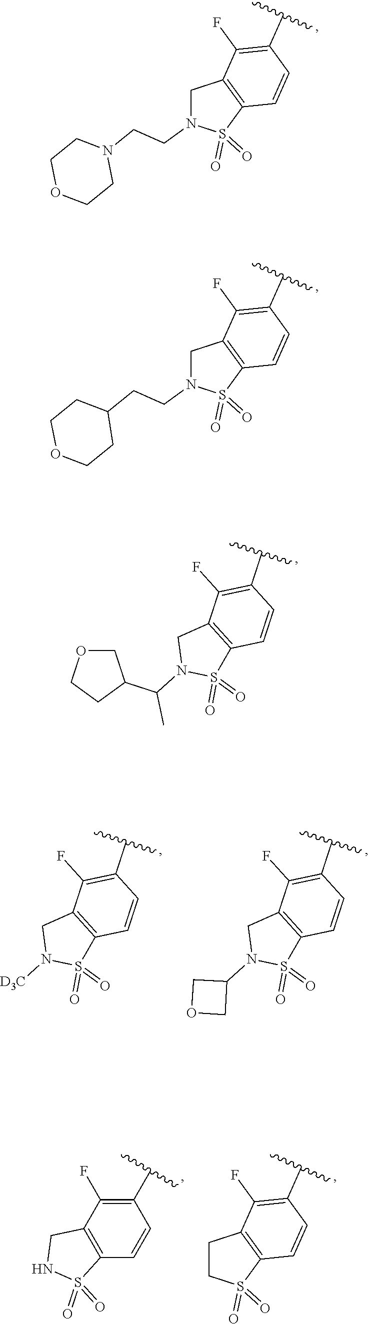

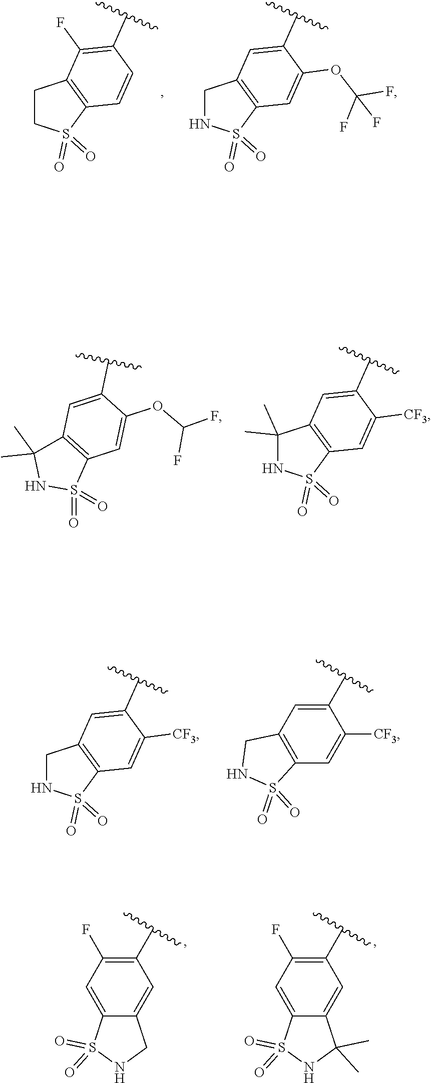

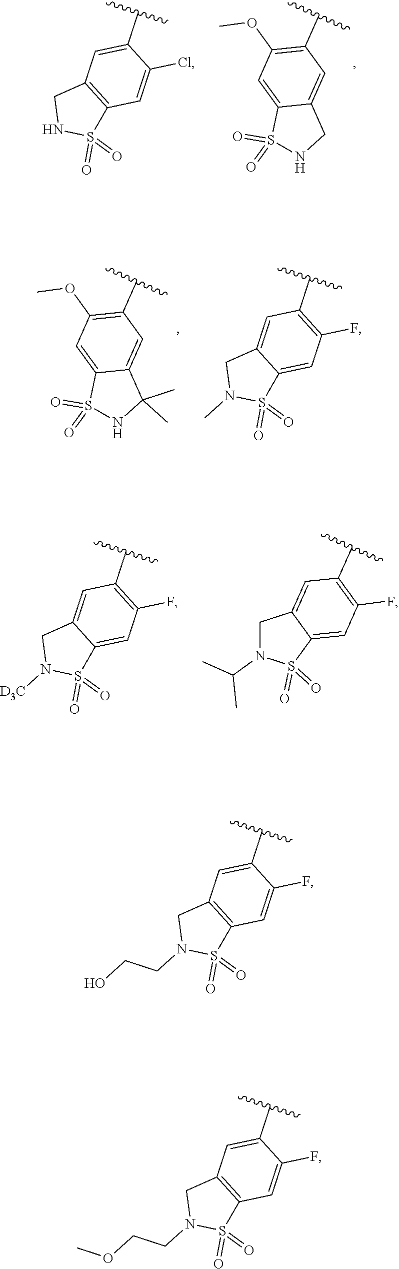

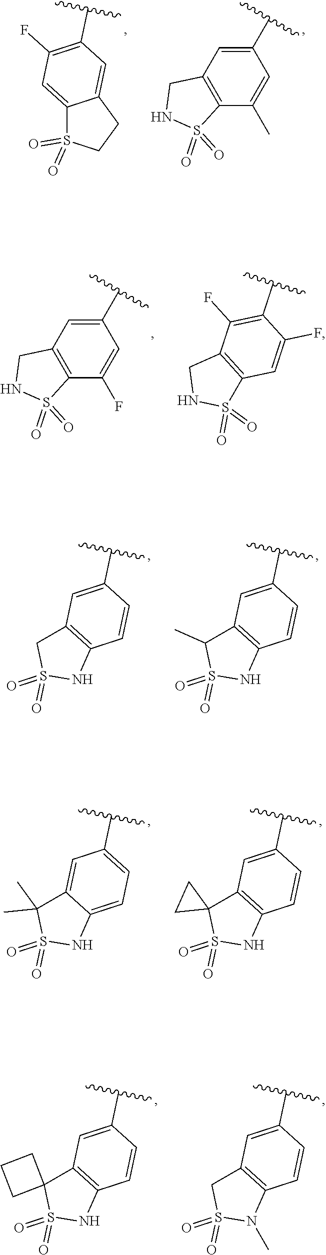

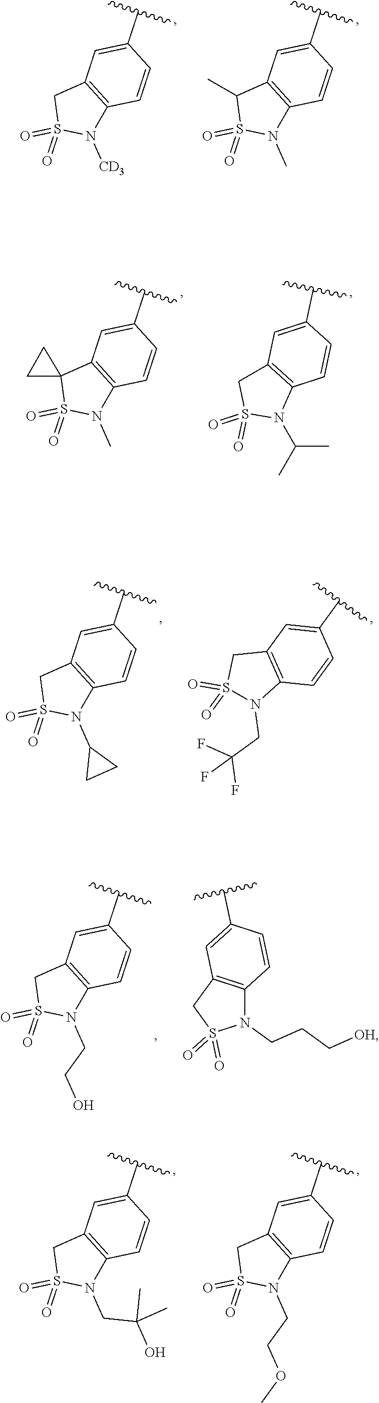

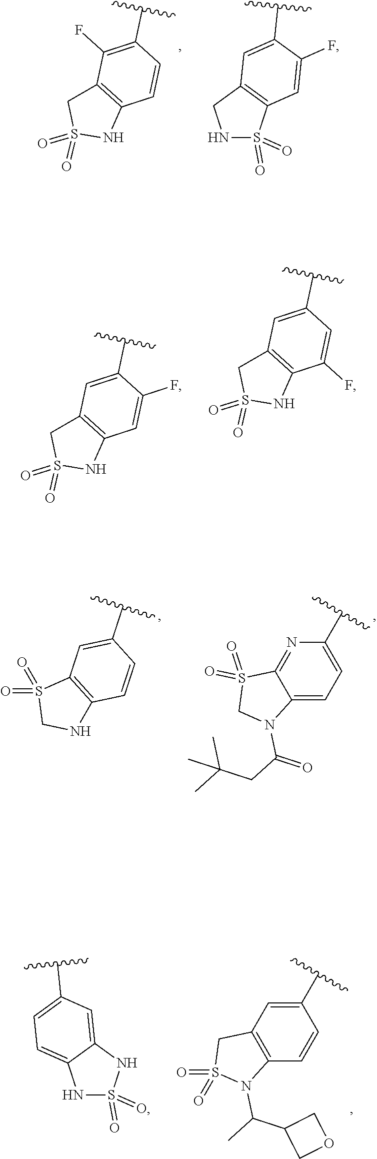

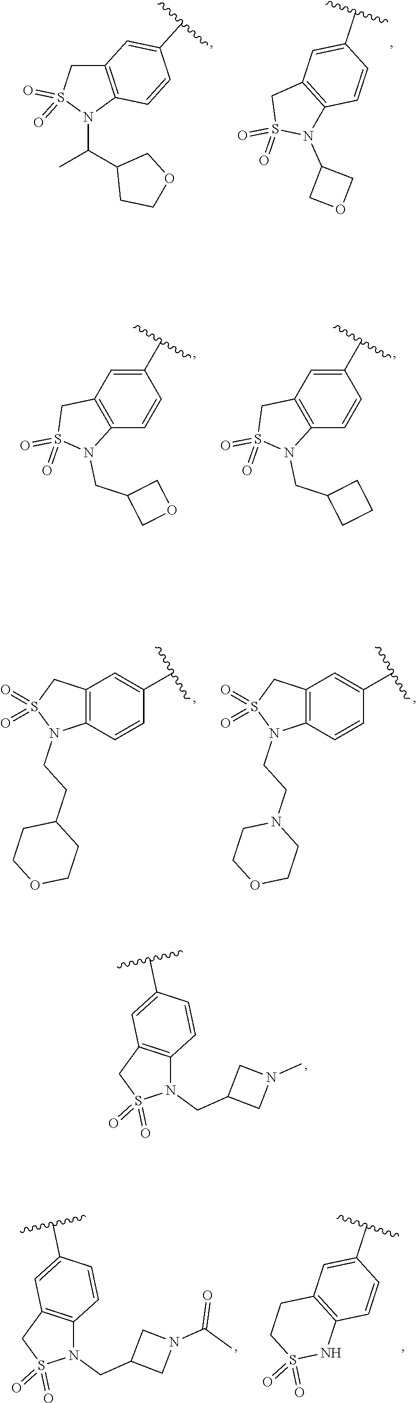

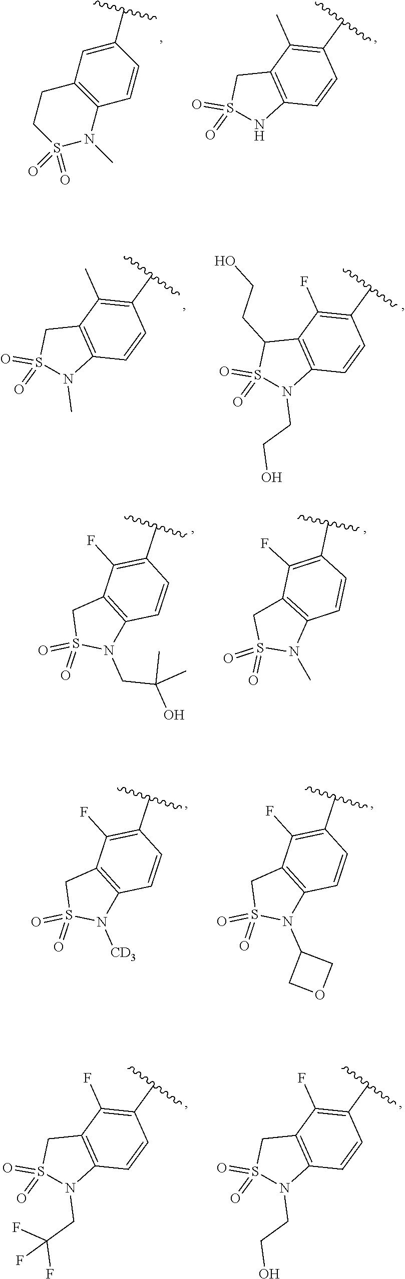

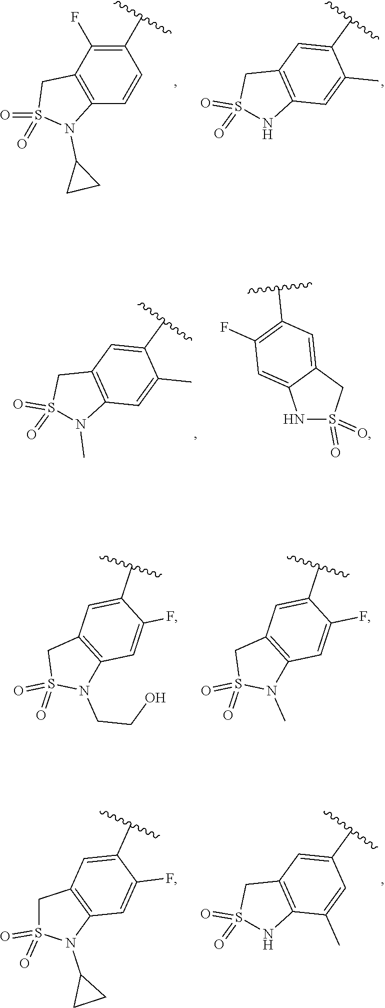

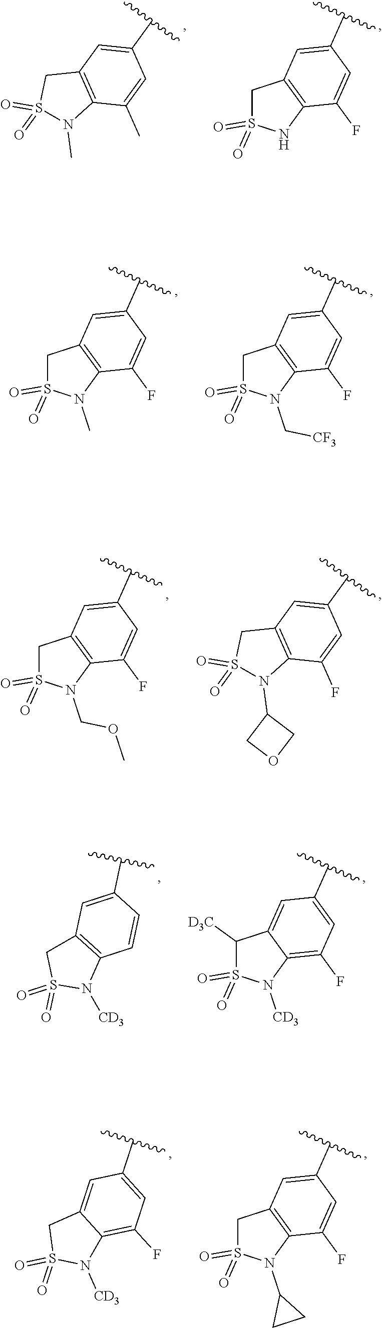

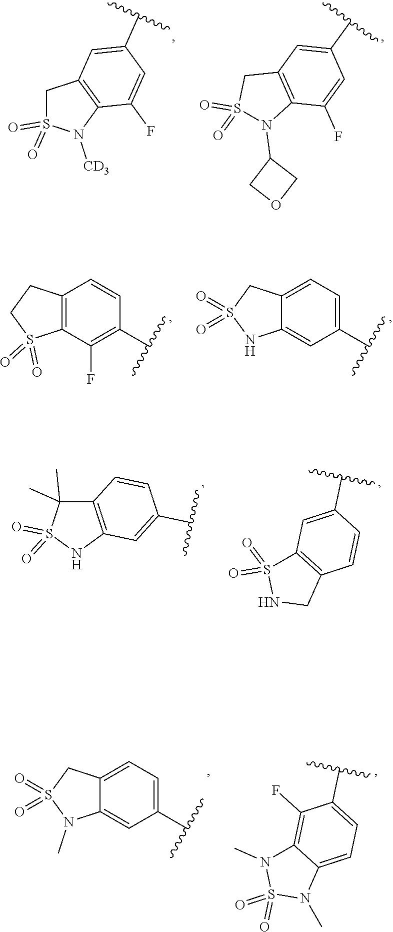

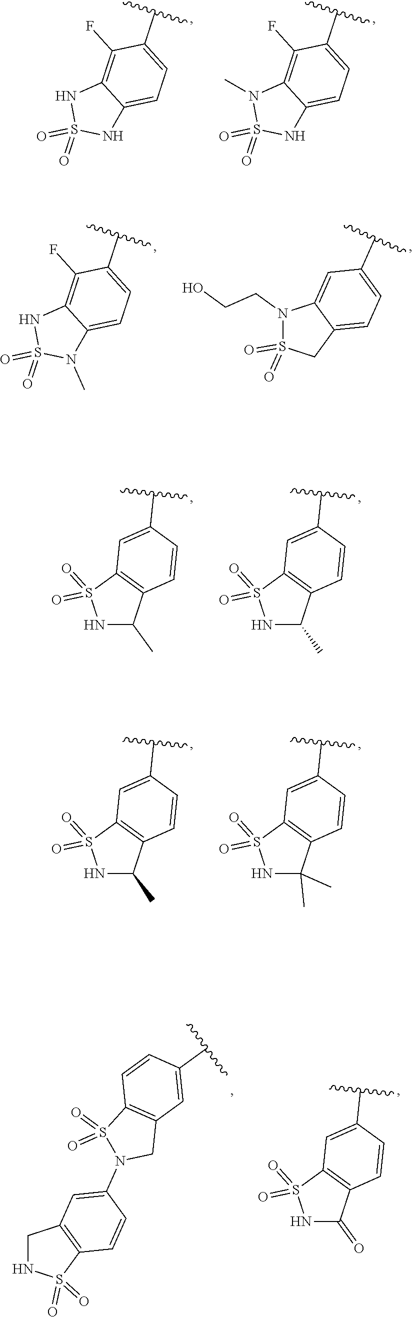

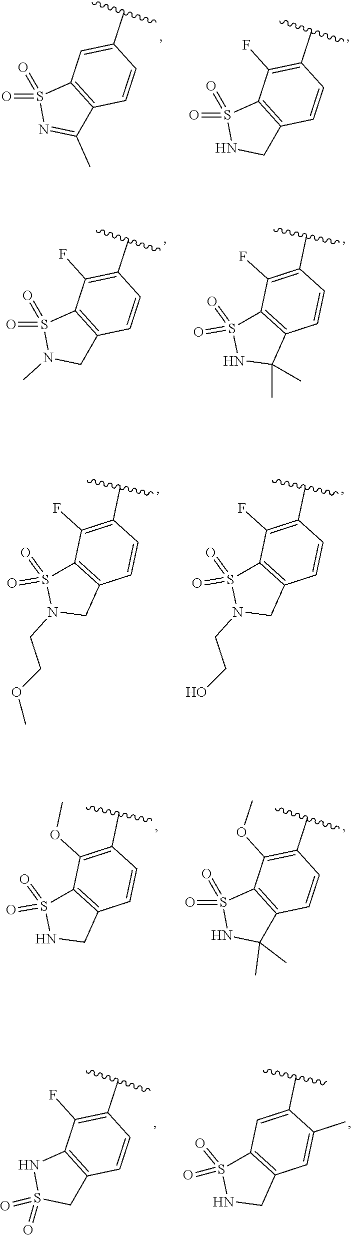

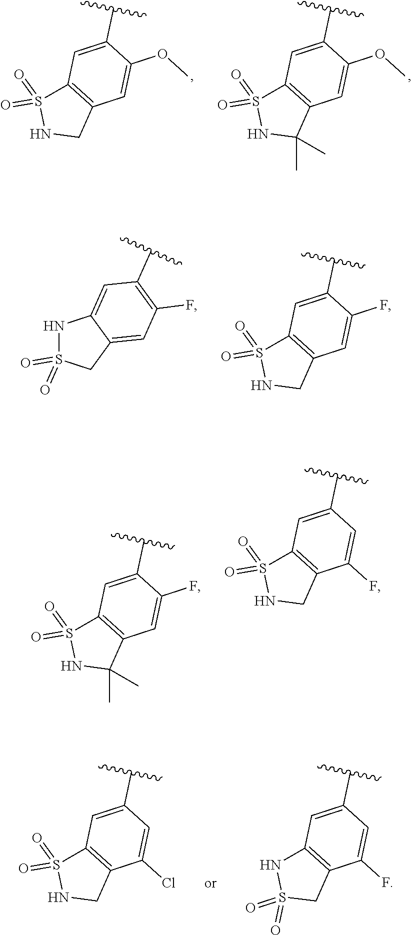

19. A compound, wherein the compound is selected from 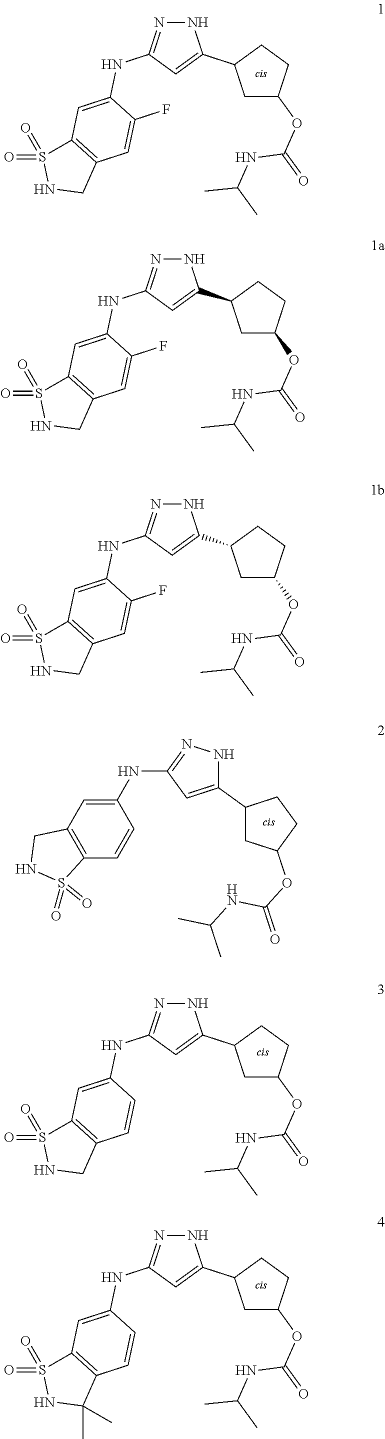 [SMILES: CC(C)NC(=O)OC1CCC(c2cc(Nc3cc4c(cc3F)CNS4(=O)=O)n[nH]2)C1 ; CC(C)NC(=O)O[C@@H]1CC[C@H](c2cc(Nc3cc4c(cc3F)CNS4(=O)=O)n[nH]2)C1 ; CC(C)NC(=O)O[C@H]1CC[C@@H](c2cc(Nc3cc4c(cc3F)CNS4(=O)=O)n[nH]2)C1 ; CC(C)NC(=O)OC1CCC(c2cc(Nc3ccc4c(c3)CNS4(=O)=O)n[nH]2)C1 ; CC(C)NC(=O)OC1CCC(c2cc(Nc3ccc4c(c3)S(=O)(=O)NC4)n[nH]2)C1 ; CC(C)NC(=O)OC1CCC(c2cc(Nc3ccc4c(c3)S(=O)(=O)NC4(C)C)n[nH]2)C1] 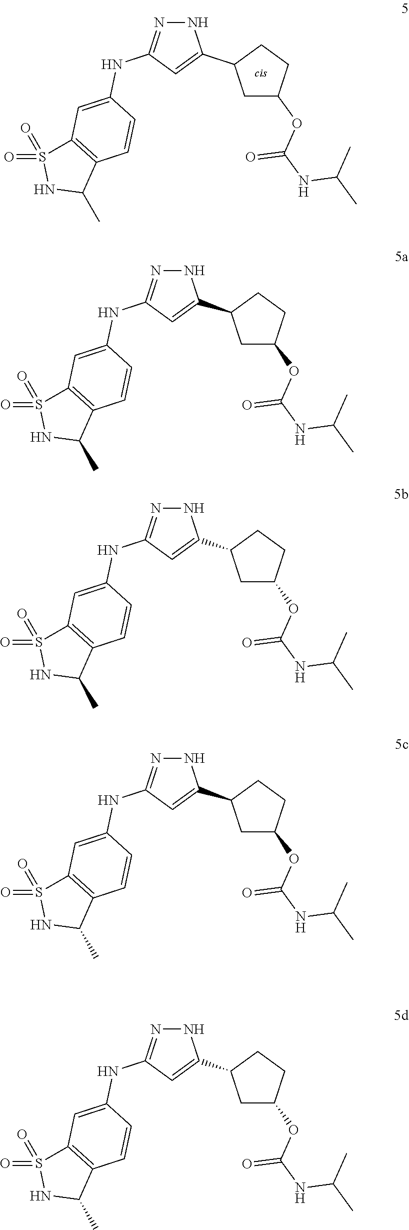 [SMILES: CC(C)NC(=O)OC1CCC(c2cc(Nc3ccc4c(c3)S(=O)(=O)NC4C)n[nH]2)C1 ; CC(C)NC(=O)O[C@@H]1CC[C@H](c2cc(Nc3ccc4c(c3)S(=O)(=O)N[C@@H]4C)n[nH]2)C1 ; CC(C)NC(=O)O[C@H]1CC[C@@H](c2cc(Nc3ccc4c(c3)S(=O)(=O)N[C@@H]4C)n[nH]2)C1 ; CC(C)NC(=O)O[C@@H]1CC[C@H](c2cc(Nc3ccc4c(c3)S(=O)(=O)N[C@H]4C)n[nH]2)C1 ; CC(C)NC(=O)O[C@H]1CC[C@@H](c2cc(Nc3ccc4c(c3)S(=O)(=O)N[C@H]4C)n[nH]2)C1] 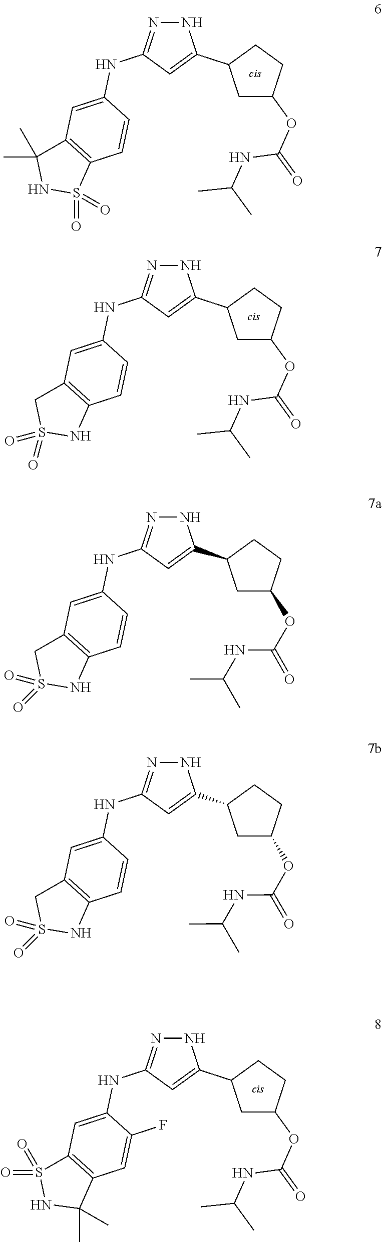 [SMILES: CC(C)NC(=O)OC1CCC(c2cc(Nc3ccc4c(c3)C(C)(C)NS4(=O)=O)n[nH]2)C1 ; CC(C)NC(=O)OC1CCC(c2cc(Nc3ccc4c(c3)CS(=O)(=O)N4)n[nH]2)C1 ; CC(C)NC(=O)O[C@@H]1CC[C@H](c2cc(Nc3ccc4c(c3)CS(=O)(=O)N4)n[nH]2)C1 ; CC(C)NC(=O)O[C@H]1CC[C@@H](c2cc(Nc3ccc4c(c3)CS(=O)(=O)N4)n[nH]2)C1 ; CC(C)NC(=O)OC1CCC(c2cc(Nc3cc4c(cc3F)C(C)(C)NS4(=O)=O)n[nH]2)C1] 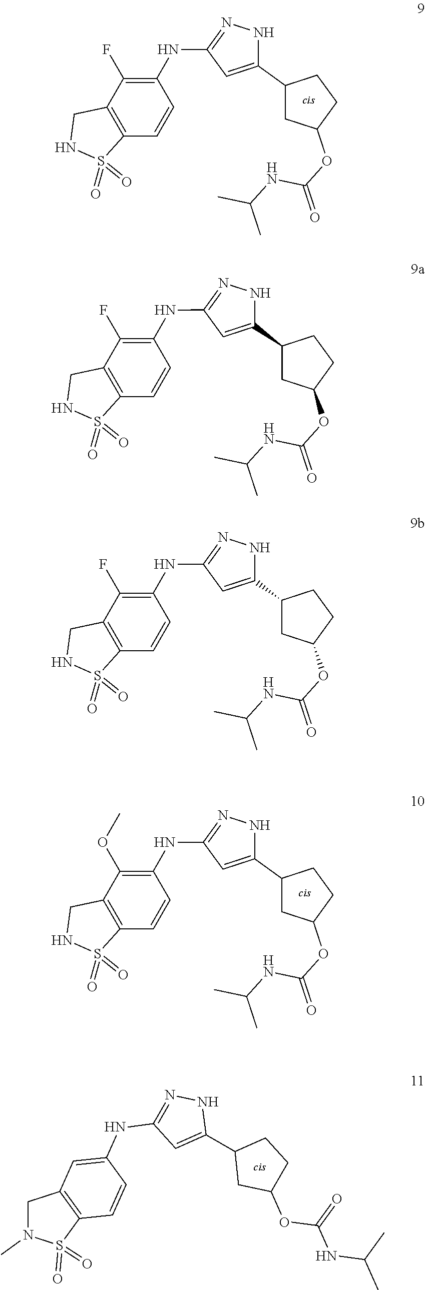 [SMILES: CC(C)NC(=O)OC1CCC(c2cc(Nc3ccc4c(c3F)CNS4(=O)=O)n[nH]2)C1 ; CC(C)NC(=O)O[C@@H]1CC[C@H](c2cc(Nc3ccc4c(c3F)CNS4(=O)=O)n[nH]2)C1 ; CC(C)NC(=O)O[C@H]1CC[C@@H](c2cc(Nc3ccc4c(c3F)CNS4(=O)=O)n[nH]2)C1 ; COc1c(Nc2cc(C3CCC(OC(=O)NC(C)C)C3)[nH]n2)ccc2c1CNS2(=O)=O ; CC(C)NC(=O)OC1CCC(c2cc(Nc3ccc4c(c3)CN(C)S4(=O)=O)n[nH]2)C1] 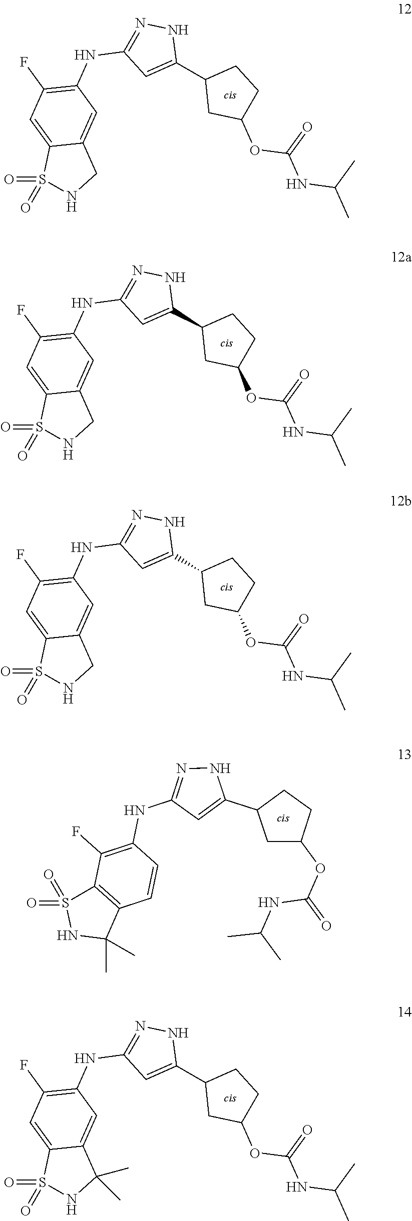 [SMILES: CC(C)NC(=O)OC1CCC(c2cc(Nc3cc4c(cc3F)S(=O)(=O)NC4)n[nH]2)C1 ; CC(C)NC(=O)O[C@@H]1CC[C@H](c2cc(Nc3cc4c(cc3F)S(=O)(=O)NC4)n[nH]2)C1 ; CC(C)NC(=O)O[C@H]1CC[C@@H](c2cc(Nc3cc4c(cc3F)S(=O)(=O)NC4)n[nH]2)C1 ; CC(C)NC(=O)OC1CCC(c2cc(Nc3ccc4c(c3F)S(=O)(=O)NC4(C)C)n[nH]2)C1 ; CC(C)NC(=O)OC1CCC(c2cc(Nc3cc4c(cc3F)S(=O)(=O)NC4(C)C)n[nH]2)C1] 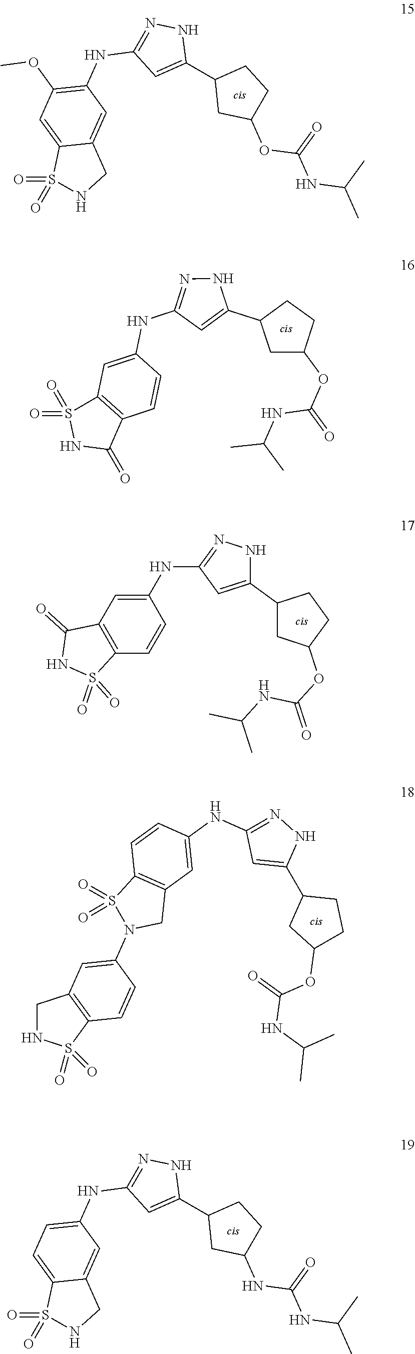 [SMILES: COc1cc2c(cc1Nc1cc(C3CCC(OC(=O)NC(C)C)C3)[nH]n1)CNS2(=O)=O ; CC(C)NC(=O)OC1CCC(c2cc(Nc3ccc4c(c3)S(=O)(=O)NC4=O)n[nH]2)C1 ; CC(C)NC(=O)OC1CCC(c2cc(Nc3ccc4c(c3)C(=O)NS4(=O)=O)n[nH]2)C1 ; CC(C)NC(=O)OC1CCC(c2cc(Nc3ccc4c(c3)CN(c3ccc5c(c3)CNS5(=O)=O)S4(=O)=O)n[nH]2)C1 ; CC(C)NC(=O)NC1CCC(c2cc(Nc3ccc4c(c3)CNS4(=O)=O)n[nH]2)C1] 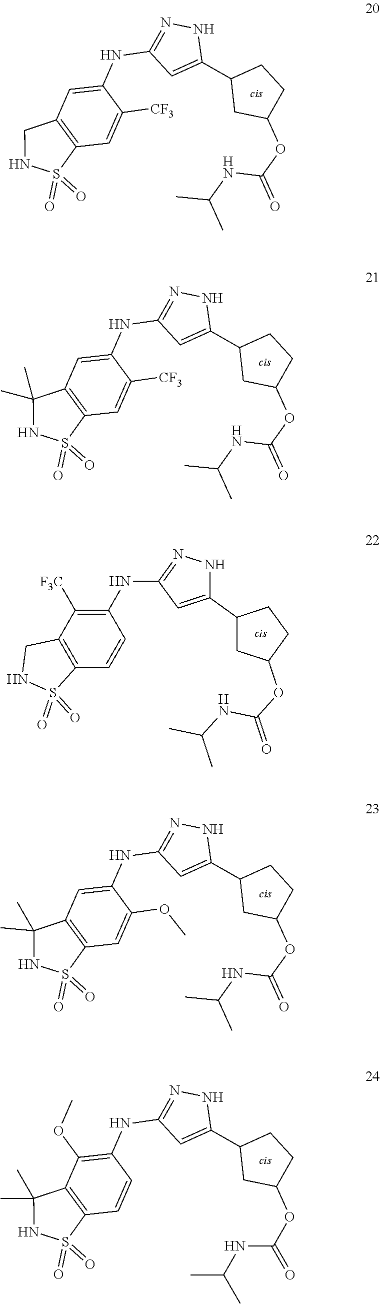 [SMILES: CC(C)NC(=O)OC1CCC(c2cc(Nc3cc4c(cc3C(F)(F)F)S(=O)(=O)NC4)n[nH]2)C1 ; CC(C)NC(=O)OC1CCC(c2cc(Nc3cc4c(cc3C(F)(F)F)S(=O)(=O)NC4(C)C)n[nH]2)C1 ; CC(C)NC(=O)OC1CCC(c2cc(Nc3ccc4c(c3C(F)(F)F)CNS4(=O)=O)n[nH]2)C1 ; COc1cc2c(cc1Nc1cc(C3CCC(OC(=O)NC(C)C)C3)[nH]n1)C(C)(C)NS2(=O)=O ; COc1c(Nc2cc(C3CCC(OC(=O)NC(C)C)C3)[nH]n2)ccc2c1C(C)(C)NS2(=O)=O] 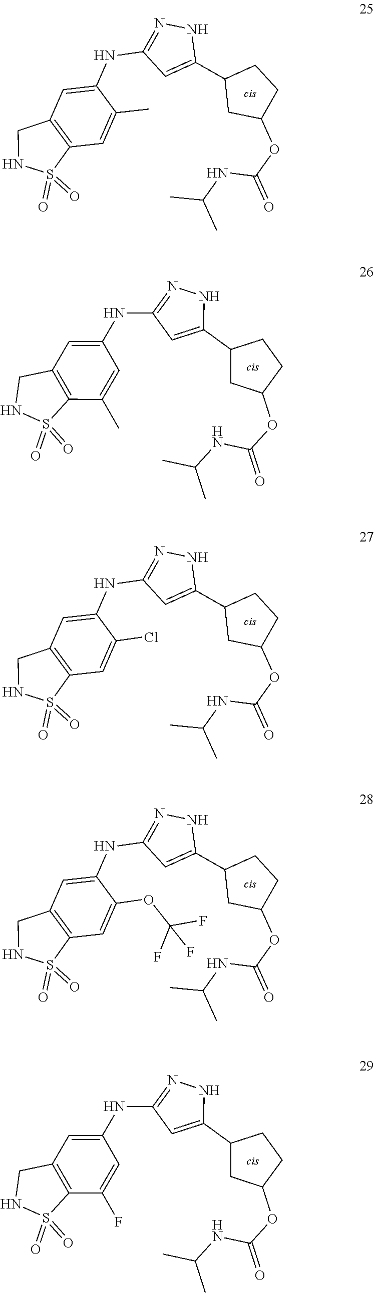 [SMILES: Cc1cc2c(cc1Nc1cc(C3CCC(OC(=O)NC(C)C)C3)[nH]n1)CNS2(=O)=O ; Cc1cc(Nc2cc(C3CCC(OC(=O)NC(C)C)C3)[nH]n2)cc2c1S(=O)(=O)NC2 ; CC(C)NC(=O)OC1CCC(c2cc(Nc3cc4c(cc3Cl)S(=O)(=O)NC4)n[nH]2)C1 ; CC(C)NC(=O)OC1CCC(c2cc(Nc3cc4c(cc3OC(F)(F)F)S(=O)(=O)NC4)n[nH]2)C1 ; CC(C)NC(=O)OC1CCC(c2cc(Nc3cc(F)c4c(c3)CNS4(=O)=O)n[nH]2)C1] 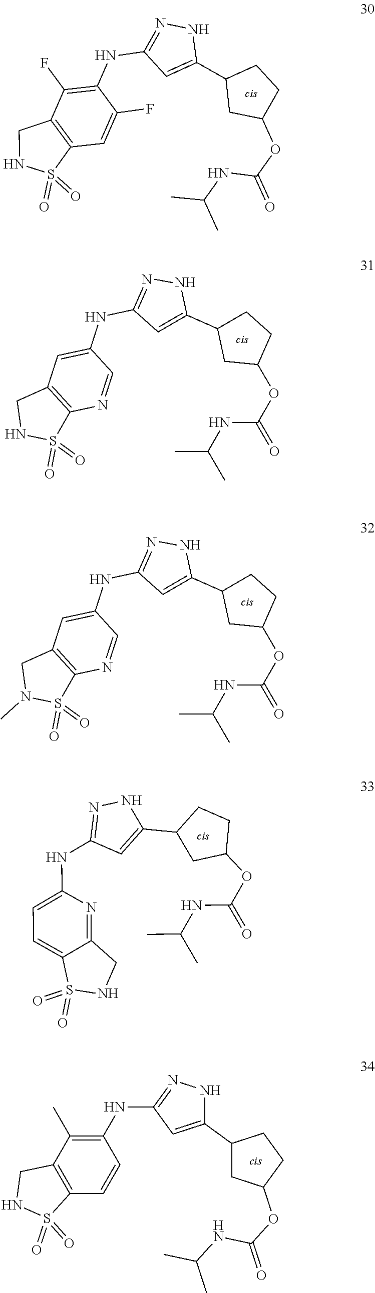 [SMILES: CC(C)NC(=O)OC1CCC(c2cc(Nc3c(F)cc4c(c3F)CNS4(=O)=O)n[nH]2)C1 ; CC(C)NC(=O)OC1CCC(c2cc(Nc3cnc4c(c3)CNS4(=O)=O)n[nH]2)C1 ; CC(C)NC(=O)OC1CCC(c2cc(Nc3cnc4c(c3)CN(C)S4(=O)=O)n[nH]2)C1 ; CC(C)NC(=O)OC1CCC(c2cc(Nc3ccc4c(n3)CNS4(=O)=O)n[nH]2)C1 ; Cc1c(Nc2cc(C3CCC(OC(=O)NC(C)C)C3)[nH]n2)ccc2c1CNS2(=O)=O] 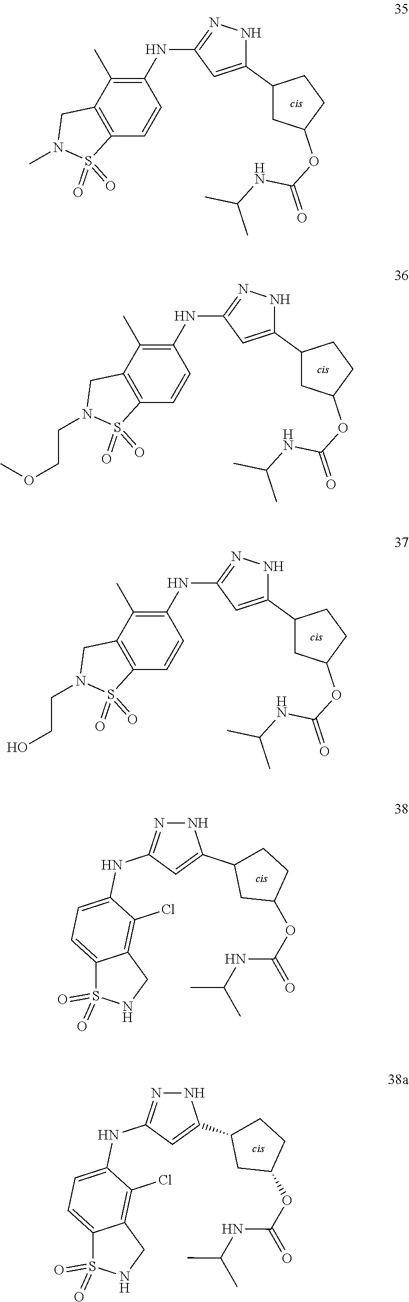 [SMILES: Cc1c(Nc2cc(C3CCC(OC(=O)NC(C)C)C3)[nH]n2)ccc2c1CN(C)S2(=O)=O ; COCCN1Cc2c(ccc(Nc3cc(C4CCC(OC(=O)NC(C)C)C4)[nH]n3)c2C)S1(=O)=O ; Cc1c(Nc2cc(C3CCC(OC(=O)NC(C)C)C3)[nH]n2)ccc2c1CN(CCO)S2(=O)=O ; CC(C)NC(=O)OC1CCC(c2cc(Nc3ccc4c(c3Cl)CNS4(=O)=O)n[nH]2)C1 ; CC(C)NC(=O)O[C@H]1CC[C@@H](c2cc(Nc3ccc4c(c3Cl)CNS4(=O)=O)n[nH]2)C1] 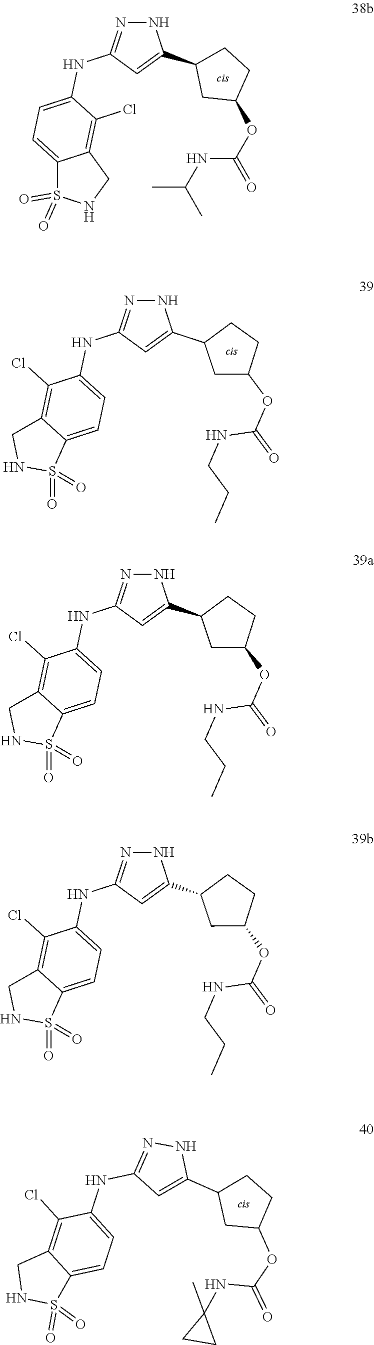 [SMILES: CC(C)NC(=O)O[C@@H]1CC[C@H](c2cc(Nc3ccc4c(c3Cl)CNS4(=O)=O)n[nH]2)C1 ; CCCNC(=O)OC1CCC(c2cc(Nc3ccc4c(c3Cl)CNS4(=O)=O)n[nH]2)C1 ; CCCNC(=O)O[C@@H]1CC[C@H](c2cc(Nc3ccc4c(c3Cl)CNS4(=O)=O)n[nH]2)C1 ; CCCNC(=O)O[C@H]1CC[C@@H](c2cc(Nc3ccc4c(c3Cl)CNS4(=O)=O)n[nH]2)C1 ; CC1(NC(=O)OC2CCC(c3cc(Nc4ccc5c(c4Cl)CNS5(=O)=O)n[nH]3)C2)CC1] 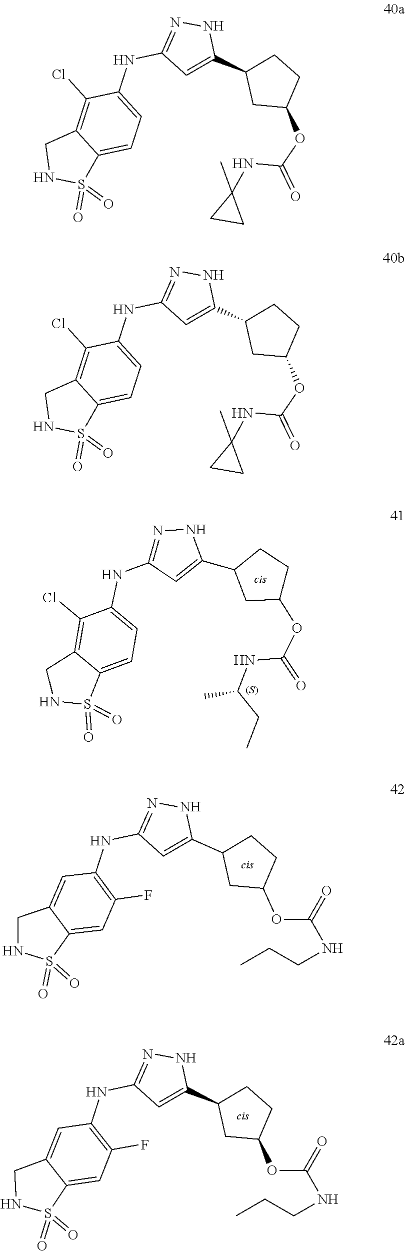 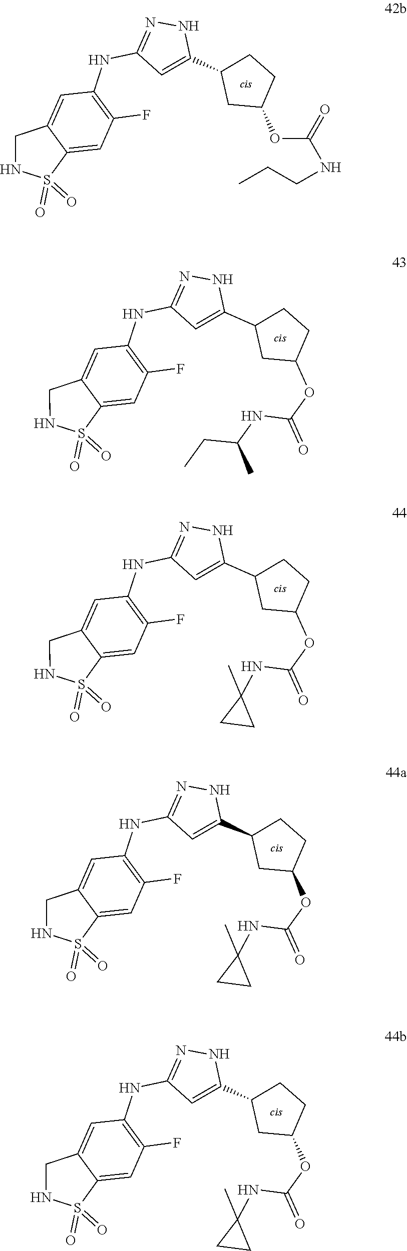 [SMILES: CCCNC(=O)O[C@H]1CC[C@@H](c2cc(Nc3cc4c(cc3F)S(=O)(=O)NC4)n[nH]2)C1 ; CC[C@H](C)NC(=O)OC1CCC(c2cc(Nc3cc4c(cc3F)S(=O)(=O)NC4)n[nH]2)C1 ; CC1(NC(=O)OC2CCC(c3cc(Nc4cc5c(cc4F)S(=O)(=O)NC5)n[nH]3)C2)CC1 ; CC1(NC(=O)O[C@@H]2CC[C@H](c3cc(Nc4cc5c(cc4F)S(=O)(=O)NC5)n[nH]3)C2)CC1 ; CC1(NC(=O)O[C@H]2CC[C@@H](c3cc(Nc4cc5c(cc4F)S(=O)(=O)NC5)n[nH]3)C2)CC1] 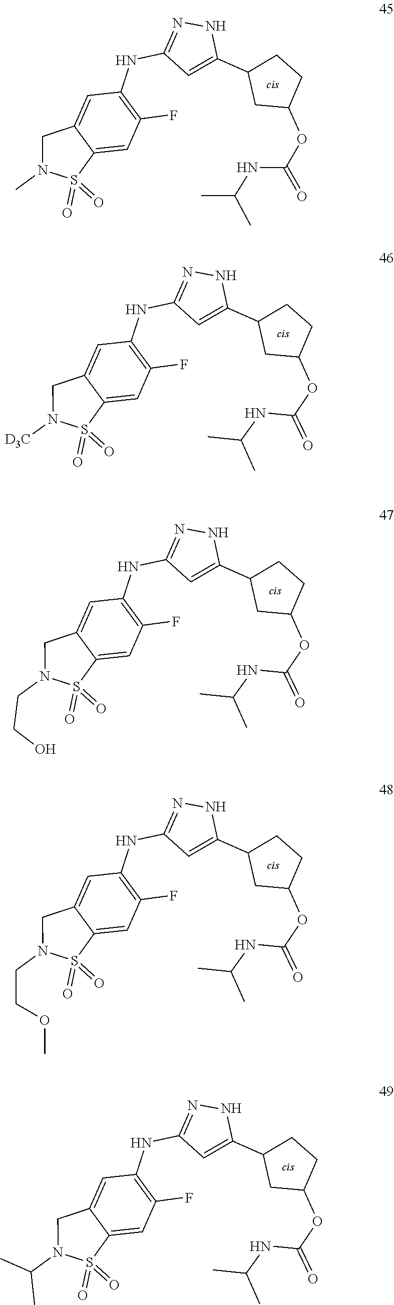 [SMILES: CC(C)NC(=O)OC1CCC(c2cc(Nc3cc4c(cc3F)S(=O)(=O)N(C)C4)n[nH]2)C1 ; [2H]C([2H])([2H])N1Cc2cc(Nc3cc(C4CCC(OC(=O)NC(C)C)C4)[nH]n3)c(F)cc2S1(=O)=O ; CC(C)NC(=O)OC1CCC(c2cc(Nc3cc4c(cc3F)S(=O)(=O)N(CCO)C4)n[nH]2)C1 ; COCCN1Cc2cc(Nc3cc(C4CCC(OC(=O)NC(C)C)C4)[nH]n3)c(F)cc2S1(=O)=O ; CC(C)NC(=O)OC1CCC(c2cc(Nc3cc4c(cc3F)S(=O)(=O)N(C(C)C)C4)n[nH]2)C1] 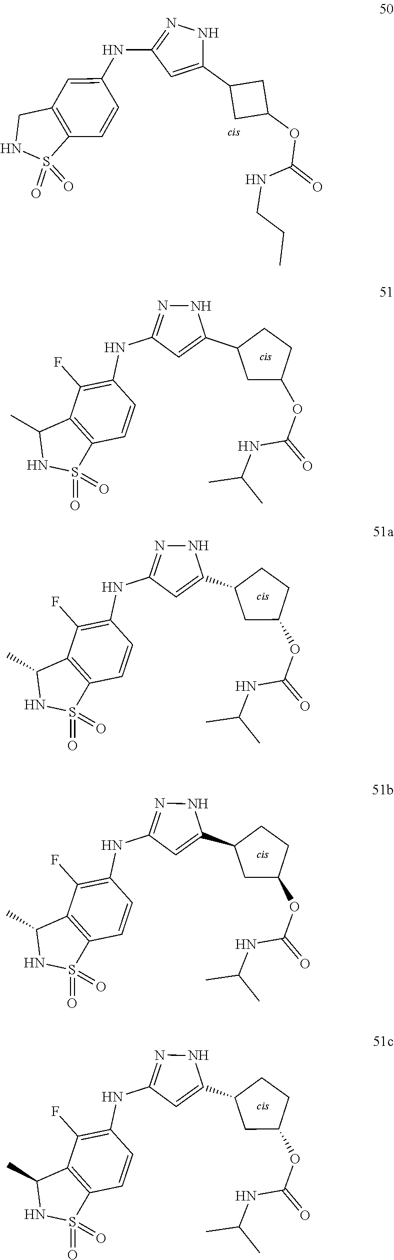 [SMILES: CCCNC(=O)OC1CC(c2cc(Nc3ccc4c(c3)CNS4(=O)=O)n[nH]2)C1 ; CC(C)NC(=O)OC1CCC(c2cc(Nc3ccc4c(c3F)C(C)NS4(=O)=O)n[nH]2)C1 ; CC(C)NC(=O)O[C@H]1CC[C@@H](c2cc(Nc3ccc4c(c3F)[C@@H](C)NS4(=O)=O)n[nH]2)C1 ; CC(C)NC(=O)O[C@@H]1CC[C@H](c2cc(Nc3ccc4c(c3F)[C@@H](C)NS4(=O)=O)n[nH]2)C1 ; CC(C)NC(=O)O[C@H]1CC[C@@H](c2cc(Nc3ccc4c(c3F)[C@H](C)NS4(=O)=O)n[nH]2)C1] 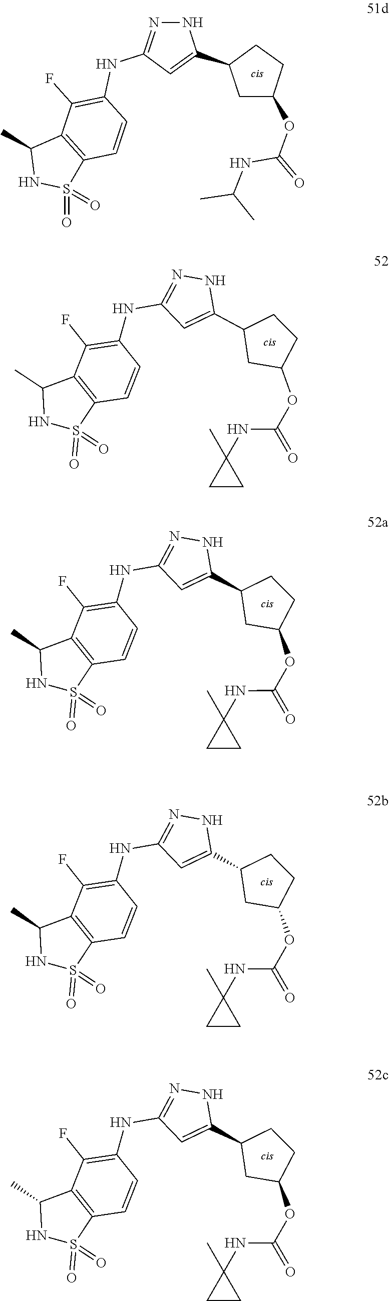 [SMILES: CC(C)NC(=O)O[C@@H]1CC[C@H](c2cc(Nc3ccc4c(c3F)[C@H](C)NS4(=O)=O)n[nH]2)C1 ; CC1NS(=O)(=O)c2ccc(Nc3cc(C4CCC(OC(=O)NC5(C)CC5)C4)[nH]n3)c(F)c21 ; C[C@@H]1NS(=O)(=O)c2ccc(Nc3cc([C@H]4CC[C@@H](OC(=O)NC5(C)CC5)C4)[nH]n3)c(F)c21 ; C[C@@H]1NS(=O)(=O)c2ccc(Nc3cc([C@@H]4CC[C@H](OC(=O)NC5(C)CC5)C4)[nH]n3)c(F)c21 ; C[C@H]1NS(=O)(=O)c2ccc(Nc3cc([C@H]4CC[C@@H](OC(=O)NC5(C)CC5)C4)[nH]n3)c(F)c21] 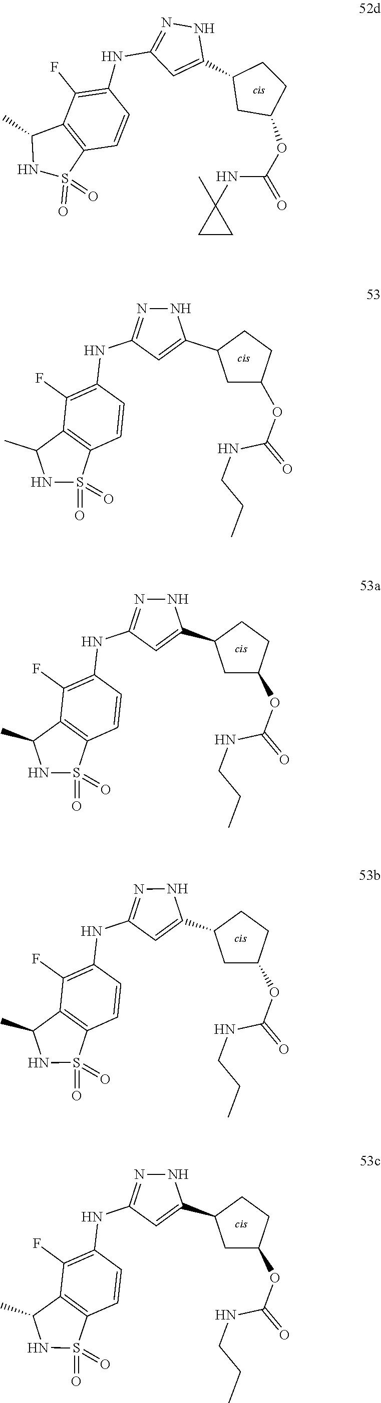 [SMILES: C[C@H]1NS(=O)(=O)c2ccc(Nc3cc([C@@H]4CC[C@H](OC(=O)NC5(C)CC5)C4)[nH]n3)c(F)c21 ; CCCNC(=O)OC1CCC(c2cc(Nc3ccc4c(c3F)C(C)NS4(=O)=O)n[nH]2)C1 ; CCCNC(=O)O[C@@H]1CC[C@H](c2cc(Nc3ccc4c(c3F)[C@H](C)NS4(=O)=O)n[nH]2)C1 ; CCCNC(=O)O[C@H]1CC[C@@H](c2cc(Nc3ccc4c(c3F)[C@H](C)NS4(=O)=O)n[nH]2)C1 ; CCCNC(=O)O[C@@H]1CC[C@H](c2cc(Nc3ccc4c(c3F)[C@@H](C)NS4(=O)=O)n[nH]2)C1] 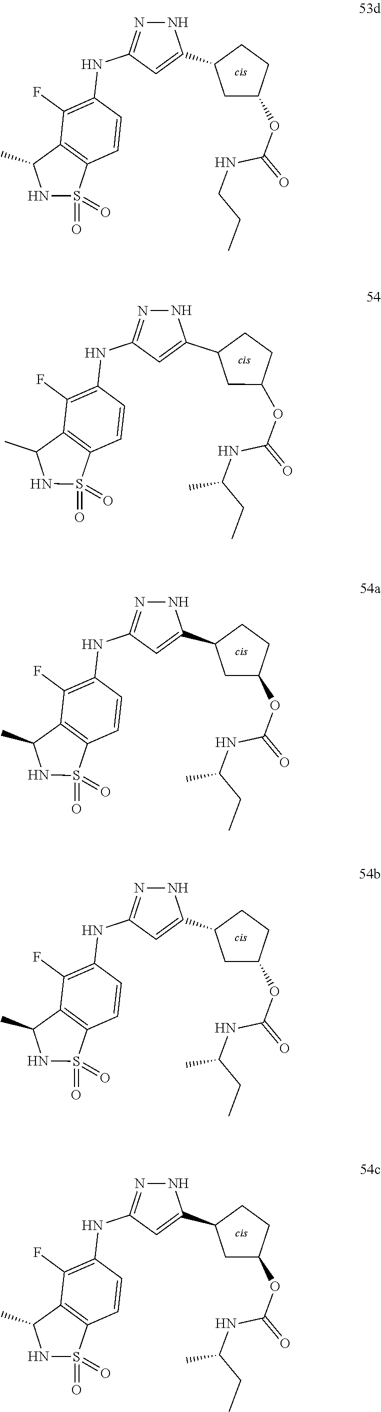 [SMILES: CCCNC(=O)O[C@H]1CC[C@@H](c2cc(Nc3ccc4c(c3F)[C@@H](C)NS4(=O)=O)n[nH]2)C1 ; CC[C@H](C)NC(=O)OC1CCC(c2cc(Nc3ccc4c(c3F)C(C)NS4(=O)=O)n[nH]2)C1 ; CC[C@H](C)NC(=O)O[C@@H]1CC[C@H](c2cc(Nc3ccc4c(c3F)[C@H](C)NS4(=O)=O)n[nH]2)C1 ; CC[C@H](C)NC(=O)O[C@H]1CC[C@@H](c2cc(Nc3ccc4c(c3F)[C@H](C)NS4(=O)=O)n[nH]2)C1 ; CC[C@H](C)NC(=O)O[C@@H]1CC[C@H](c2cc(Nc3ccc4c(c3F)[C@@H](C)NS4(=O)=O)n[nH]2)C1] 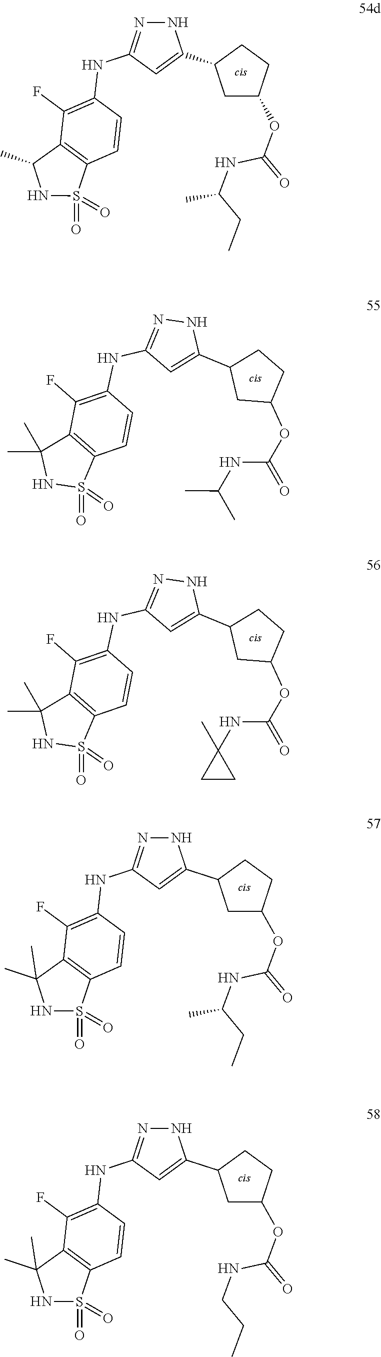 [SMILES: CC[C@H](C)NC(=O)O[C@H]1CC[C@@H](c2cc(Nc3ccc4c(c3F)[C@@H](C)NS4(=O)=O)n[nH]2)C1 ; CC(C)NC(=O)OC1CCC(c2cc(Nc3ccc4c(c3F)C(C)(C)NS4(=O)=O)n[nH]2)C1 ; CC1(NC(=O)OC2CCC(c3cc(Nc4ccc5c(c4F)C(C)(C)NS5(=O)=O)n[nH]3)C2)CC1 ; CC[C@H](C)NC(=O)OC1CCC(c2cc(Nc3ccc4c(c3F)C(C)(C)NS4(=O)=O)n[nH]2)C1 ; CCCNC(=O)OC1CCC(c2cc(Nc3ccc4c(c3F)C(C)(C)NS4(=O)=O)n[nH]2)C1] 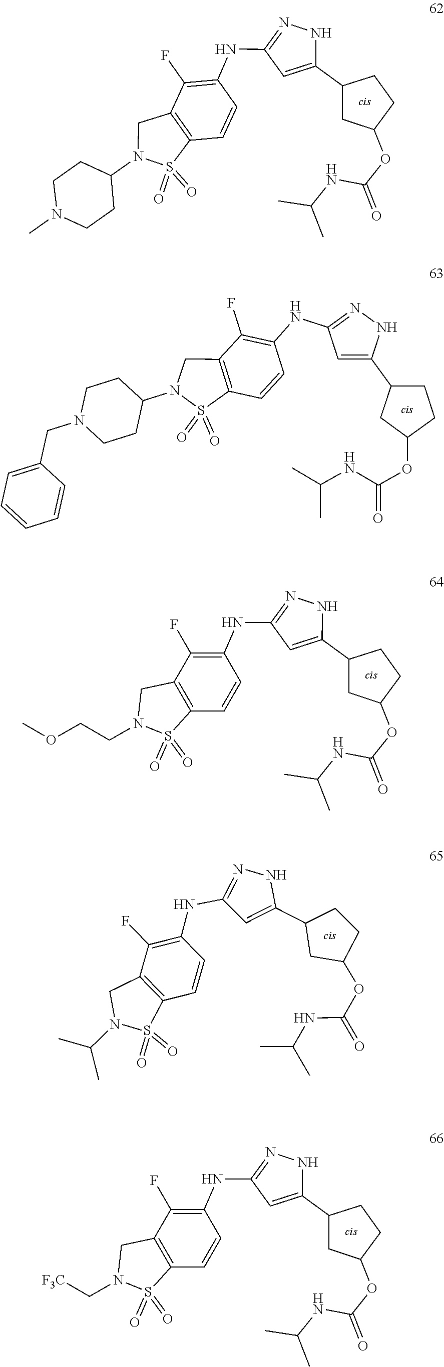 [SMILES: CC(C)NC(=O)OC1CCC(c2cc(Nc3ccc4c(c3F)CN(C3CCN(C)CC3)S4(=O)=O)n[nH]2)C1 ; CC(C)NC(=O)OC1CCC(c2cc(Nc3ccc4c(c3F)CN(C3CCN(Cc5ccccc5)CC3)S4(=O)=O)n[nH]2)C1 ; COCCN1Cc2c(ccc(Nc3cc(C4CCC(OC(=O)NC(C)C)C4)[nH]n3)c2F)S1(=O)=O ; CC(C)NC(=O)OC1CCC(c2cc(Nc3ccc4c(c3F)CN(C(C)C)S4(=O)=O)n[nH]2)C1 ; CC(C)NC(=O)OC1CCC(c2cc(Nc3ccc4c(c3F)CN(CC(F)(F)F)S4(=O)=O)n[nH]2)C1] 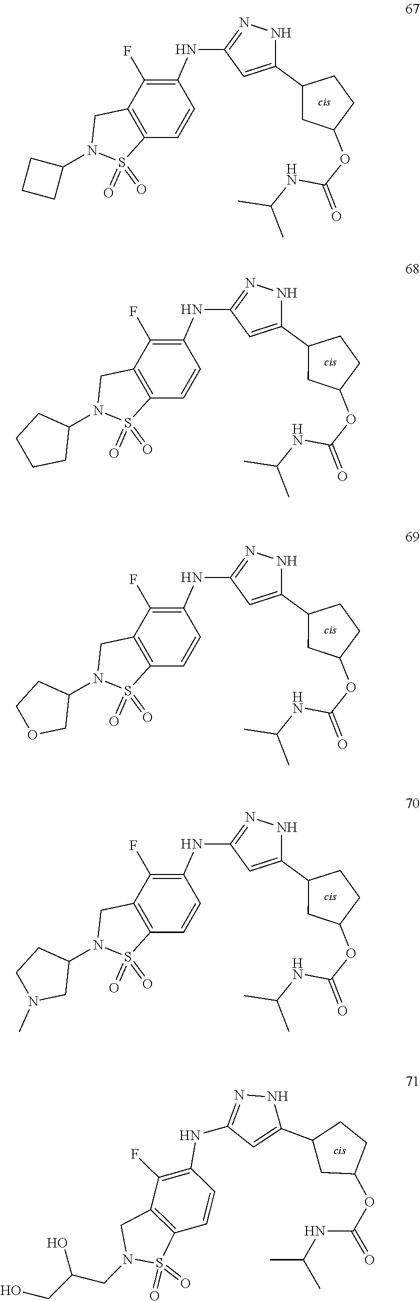 [SMILES: CC(C)NC(=O)OC1CCC(c2cc(Nc3ccc4c(c3F)CN(C3CCC3)S4(=O)=O)n[nH]2)C1 ; CC(C)NC(=O)OC1CCC(c2cc(Nc3ccc4c(c3F)CN(C3CCCC3)S4(=O)=O)n[nH]2)C1 ; CC(C)NC(=O)OC1CCC(c2cc(Nc3ccc4c(c3F)CN(C3CCOC3)S4(=O)=O)n[nH]2)C1 ; CC(C)NC(=O)OC1CCC(c2cc(Nc3ccc4c(c3F)CN(C3CCN(C)C3)S4(=O)=O)n[nH]2)C1 ; CC(C)NC(=O)OC1CCC(c2cc(Nc3ccc4c(c3F)CN(CC(O)CO)S4(=O)=O)n[nH]2)C1] 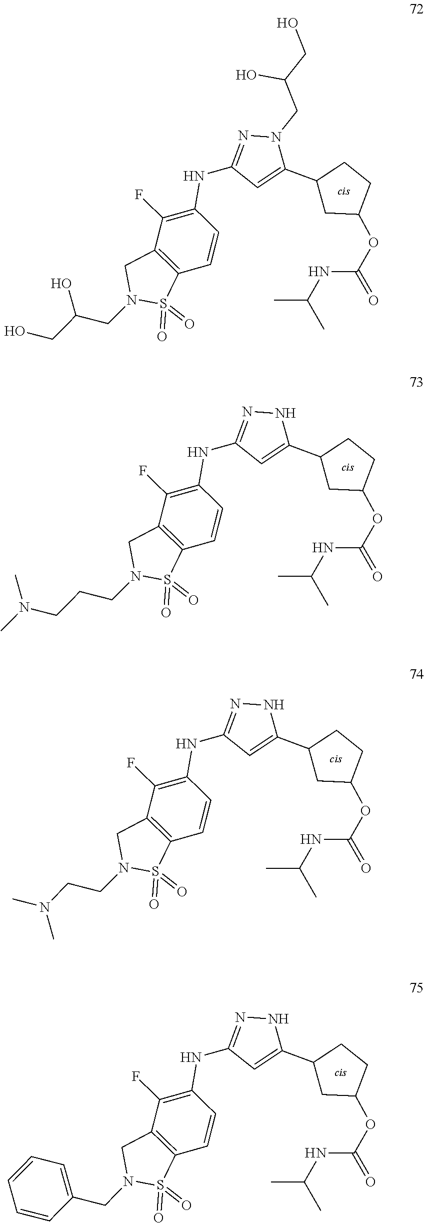 [SMILES: CC(C)NC(=O)OC1CCC(c2cc(Nc3ccc4c(c3F)CN(CC(O)CO)S4(=O)=O)nn2CC(O)CO)C1 ; CC(C)NC(=O)OC1CCC(c2cc(Nc3ccc4c(c3F)CN(CCCN(C)C)S4(=O)=O)n[nH]2)C1 ; CC(C)NC(=O)OC1CCC(c2cc(Nc3ccc4c(c3F)CN(CCN(C)C)S4(=O)=O)n[nH]2)C1 ; CC(C)NC(=O)OC1CCC(c2cc(Nc3ccc4c(c3F)CN(Cc3ccccc3)S4(=O)=O)n[nH]2)C1] 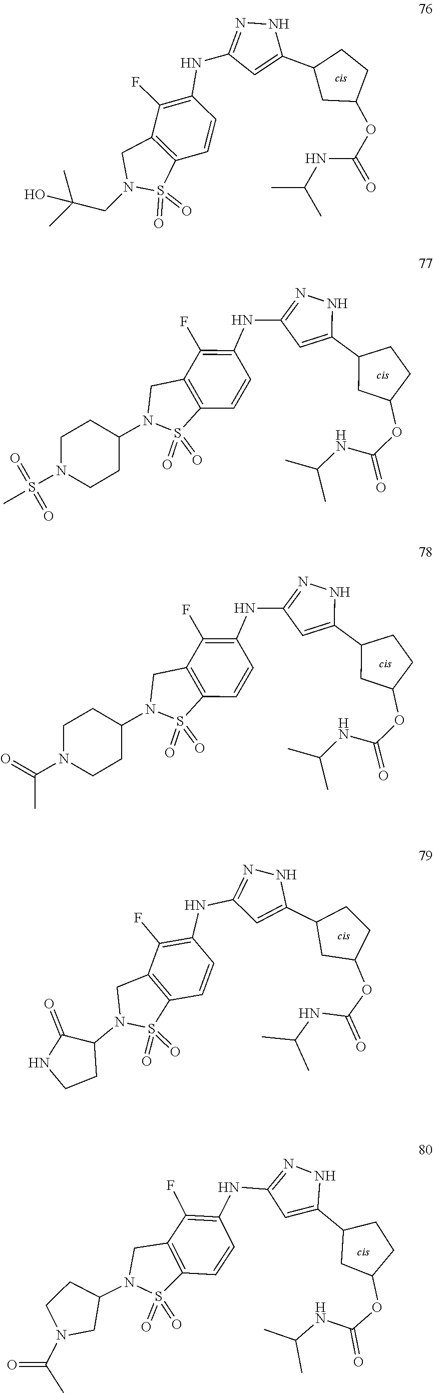 [SMILES: CC(C)NC(=O)OC1CCC(c2cc(Nc3ccc4c(c3F)CN(CC(C)(C)O)S4(=O)=O)n[nH]2)C1 ; CC(C)NC(=O)OC1CCC(c2cc(Nc3ccc4c(c3F)CN(C3CCN(S(C)(=O)=O)CC3)S4(=O)=O)n[nH]2)C1 ; CC(=O)N1CCC(N2Cc3c(ccc(Nc4cc(C5CCC(OC(=O)NC(C)C)C5)[nH]n4)c3F)S2(=O)=O)CC1 ; CC(C)NC(=O)OC1CCC(c2cc(Nc3ccc4c(c3F)CN(C3CCNC3=O)S4(=O)=O)n[nH]2)C1 ; CC(=O)N1CCC(N2Cc3c(ccc(Nc4cc(C5CCC(OC(=O)NC(C)C)C5)[nH]n4)c3F)S2(=O)=O)C1] 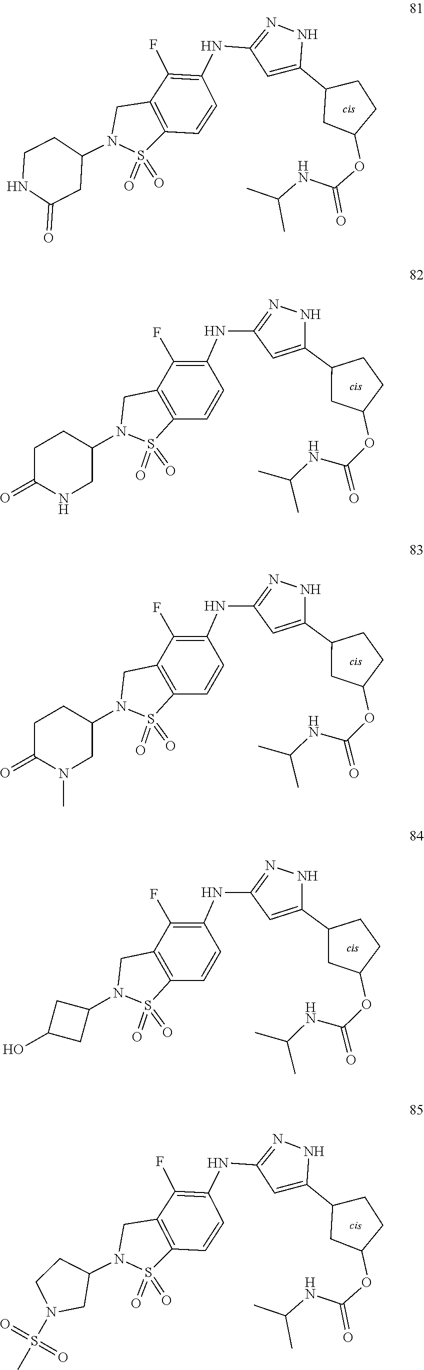 [SMILES: CC(C)NC(=O)OC1CCC(c2cc(Nc3ccc4c(c3F)CN(C3CCNC(=O)C3)S4(=O)=O)n[nH]2)C1 ; CC(C)NC(=O)OC1CCC(c2cc(Nc3ccc4c(c3F)CN(C3CCC(=O)NC3)S4(=O)=O)n[nH]2)C1 ; CC(C)NC(=O)OC1CCC(c2cc(Nc3ccc4c(c3F)CN(C3CCC(=O)N(C)C3)S4(=O)=O)n[nH]2)C1 ; CC(C)NC(=O)OC1CCC(c2cc(Nc3ccc4c(c3F)CN(C3CC(O)C3)S4(=O)=O)n[nH]2)C1 ; CC(C)NC(=O)OC1CCC(c2cc(Nc3ccc4c(c3F)CN(C3CCN(S(C)(=O)=O)C3)S4(=O)=O)n[nH]2)C1] 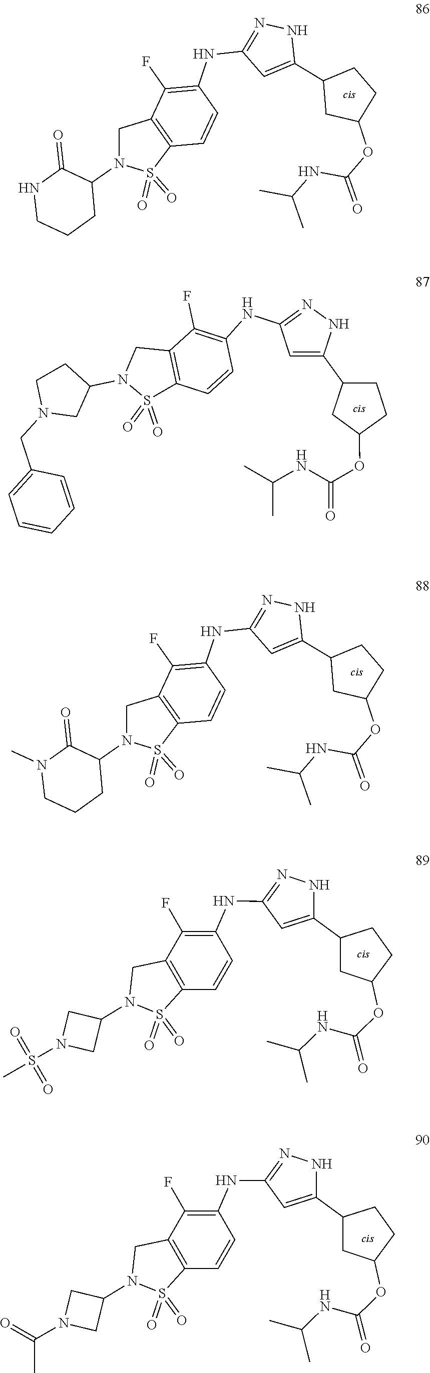 [SMILES: CC(C)NC(=O)OC1CCC(c2cc(Nc3ccc4c(c3F)CN(C3CCCNC3=O)S4(=O)=O)n[nH]2)C1 ; CC(C)NC(=O)OC1CCC(c2cc(Nc3ccc4c(c3F)CN(C3CCN(Cc5ccccc5)C3)S4(=O)=O)n[nH]2)C1 ; CC(C)NC(=O)OC1CCC(c2cc(Nc3ccc4c(c3F)CN(C3CCCN(C)C3=O)S4(=O)=O)n[nH]2)C1 ; CC(C)NC(=O)OC1CCC(c2cc(Nc3ccc4c(c3F)CN(C3CN(S(C)(=O)=O)C3)S4(=O)=O)n[nH]2)C1 ; CC(=O)N1CC(N2Cc3c(ccc(Nc4cc(C5CCC(OC(=O)NC(C)C)C5)[nH]n4)c3F)S2(=O)=O)C1] 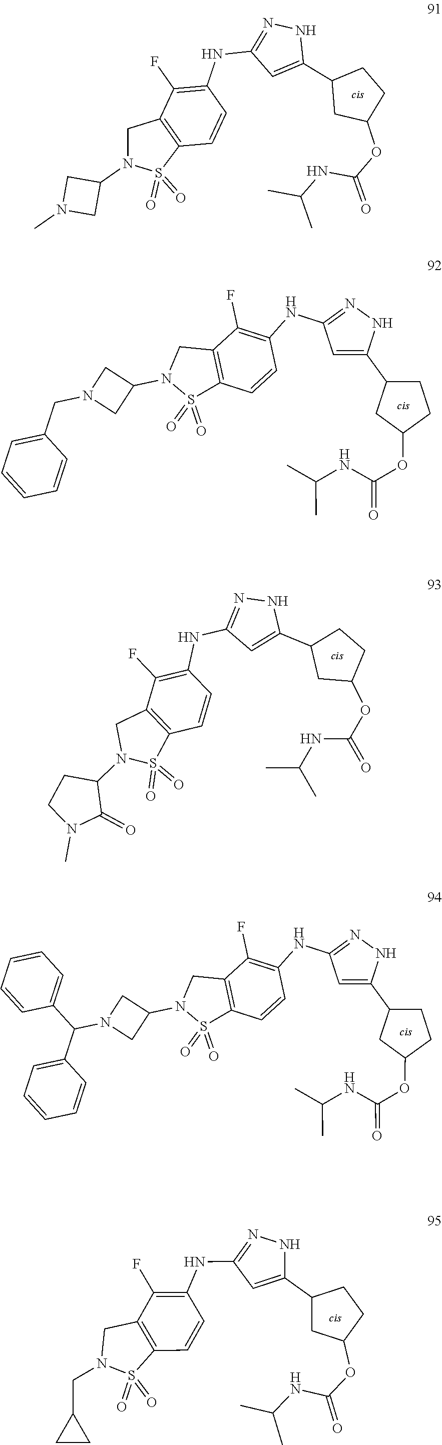 [SMILES: CC(C)NC(=O)OC1CCC(c2cc(Nc3ccc4c(c3F)CN(C3CN(C)C3)S4(=O)=O)n[nH]2)C1 ; CC(C)NC(=O)OC1CCC(c2cc(Nc3ccc4c(c3F)CN(C3CN(Cc5ccccc5)C3)S4(=O)=O)n[nH]2)C1 ; CC(C)NC(=O)OC1CCC(c2cc(Nc3ccc4c(c3F)CN(C3CCN(C)C3=O)S4(=O)=O)n[nH]2)C1 ; CC(C)NC(=O)OC1CCC(c2cc(Nc3ccc4c(c3F)CN(C3CN(C(c5ccccc5)c5ccccc5)C3)S4(=O)=O)n[nH]2)C1 ; CC(C)NC(=O)OC1CCC(c2cc(Nc3ccc4c(c3F)CN(CC3CC3)S4(=O)=O)n[nH]2)C1] 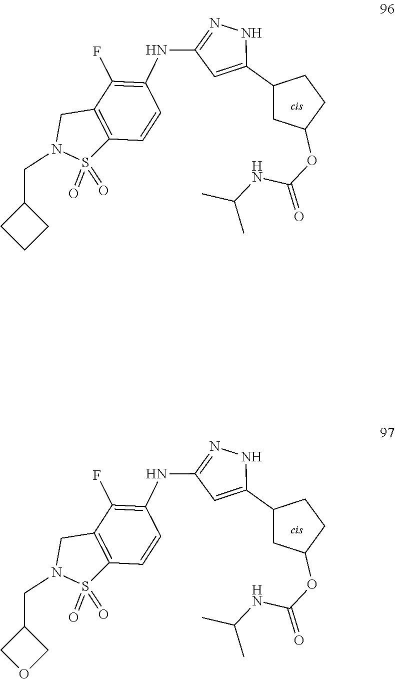 [SMILES: CC(C)NC(=O)OC1CCC(c2cc(Nc3ccc4c(c3F)CN(CC3CCC3)S4(=O)=O)n[nH]2)C1 ; CC(C)NC(=O)OC1CCC(c2cc(Nc3ccc4c(c3F)CN(CC3COC3)S4(=O)=O)n[nH]2)C1] 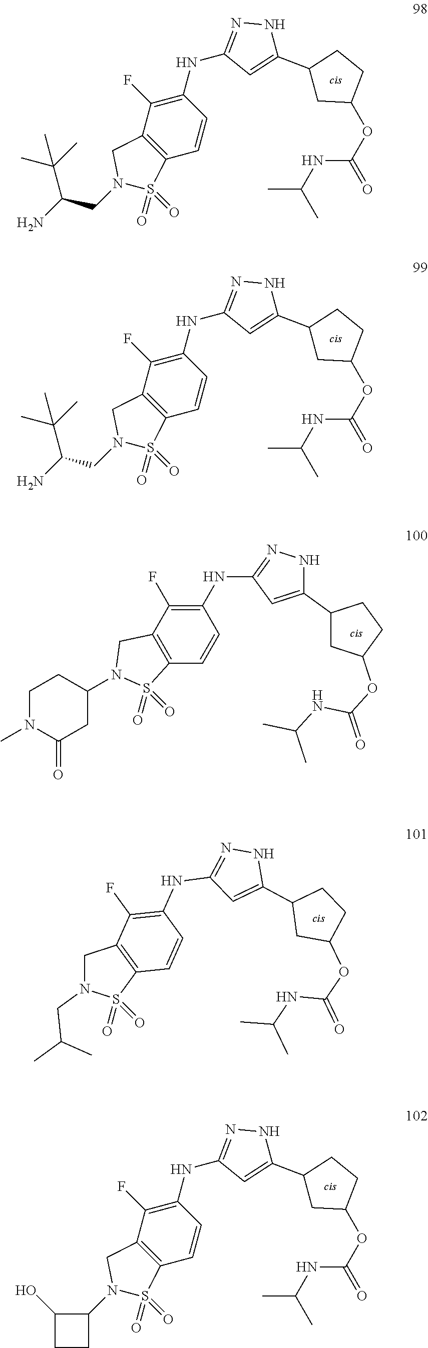 [SMILES: CC(C)NC(=O)OC1CCC(c2cc(Nc3ccc4c(c3F)CN(C[C@@H](N)C(C)(C)C)S4(=O)=O)n[nH]2)C1 ; CC(C)NC(=O)OC1CCC(c2cc(Nc3ccc4c(c3F)CN(C[C@H](N)C(C)(C)C)S4(=O)=O)n[nH]2)C1 ; CC(C)NC(=O)OC1CCC(c2cc(Nc3ccc4c(c3F)CN(C3CCN(C)C(=O)C3)S4(=O)=O)n[nH]2)C1 ; CC(C)CN1Cc2c(ccc(Nc3cc(C4CCC(OC(=O)NC(C)C)C4)[nH]n3)c2F)S1(=O)=O ; CC(C)NC(=O)OC1CCC(c2cc(Nc3ccc4c(c3F)CN(C3CCC3O)S4(=O)=O)n[nH]2)C1] 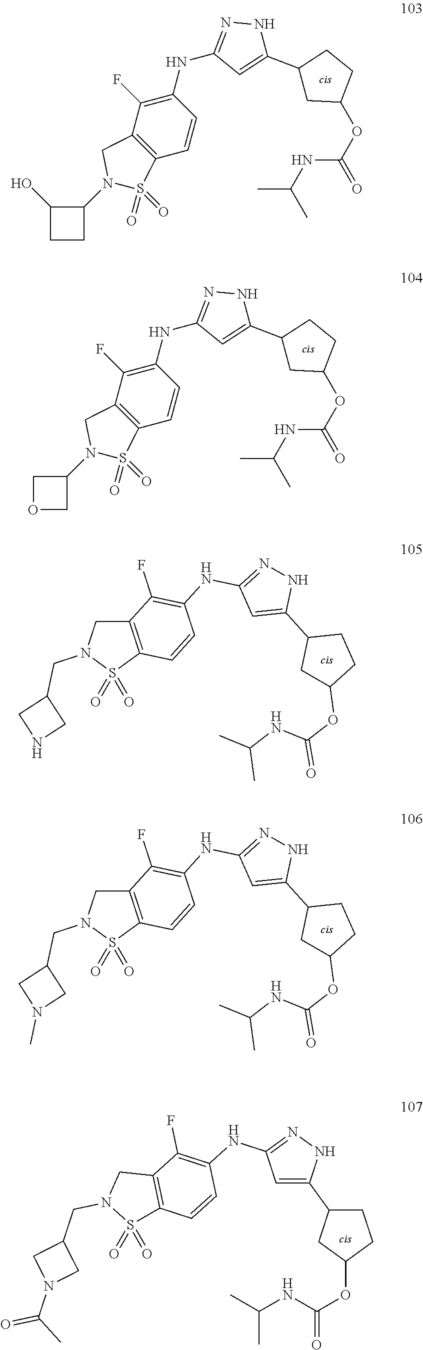 [SMILES: CC(C)NC(=O)OC1CCC(c2cc(Nc3ccc4c(c3F)CN(C3CCC3O)S4(=O)=O)n[nH]2)C1 ; CC(C)NC(=O)OC1CCC(c2cc(Nc3ccc4c(c3F)CN(C3COC3)S4(=O)=O)n[nH]2)C1 ; CC(C)NC(=O)OC1CCC(c2cc(Nc3ccc4c(c3F)CN(CC3CNC3)S4(=O)=O)n[nH]2)C1 ; CC(C)NC(=O)OC1CCC(c2cc(Nc3ccc4c(c3F)CN(CC3CN(C)C3)S4(=O)=O)n[nH]2)C1 ; CC(=O)N1CC(CN2Cc3c(ccc(Nc4cc(C5CCC(OC(=O)NC(C)C)C5)[nH]n4)c3F)S2(=O)=O)C1] 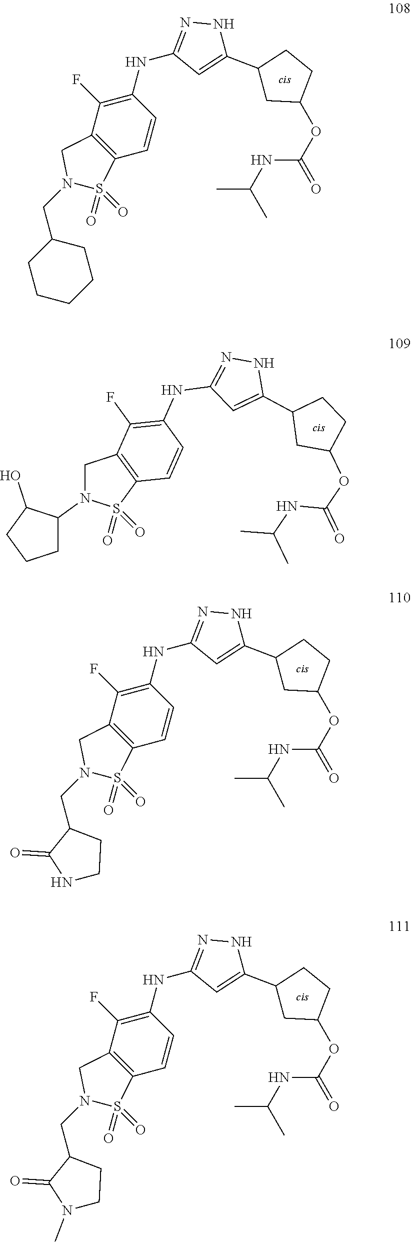 [SMILES: CC(C)NC(=O)OC1CCC(c2cc(Nc3ccc4c(c3F)CN(CC3CCCCC3)S4(=O)=O)n[nH]2)C1 ; CC(C)NC(=O)OC1CCC(c2cc(Nc3ccc4c(c3F)CN(C3CCCC3O)S4(=O)=O)n[nH]2)C1 ; CC(C)NC(=O)OC1CCC(c2cc(Nc3ccc4c(c3F)CN(CC3CCNC3=O)S4(=O)=O)n[nH]2)C1 ; CC(C)NC(=O)OC1CCC(c2cc(Nc3ccc4c(c3F)CN(CC3CCN(C)C3=O)S4(=O)=O)n[nH]2)C1] 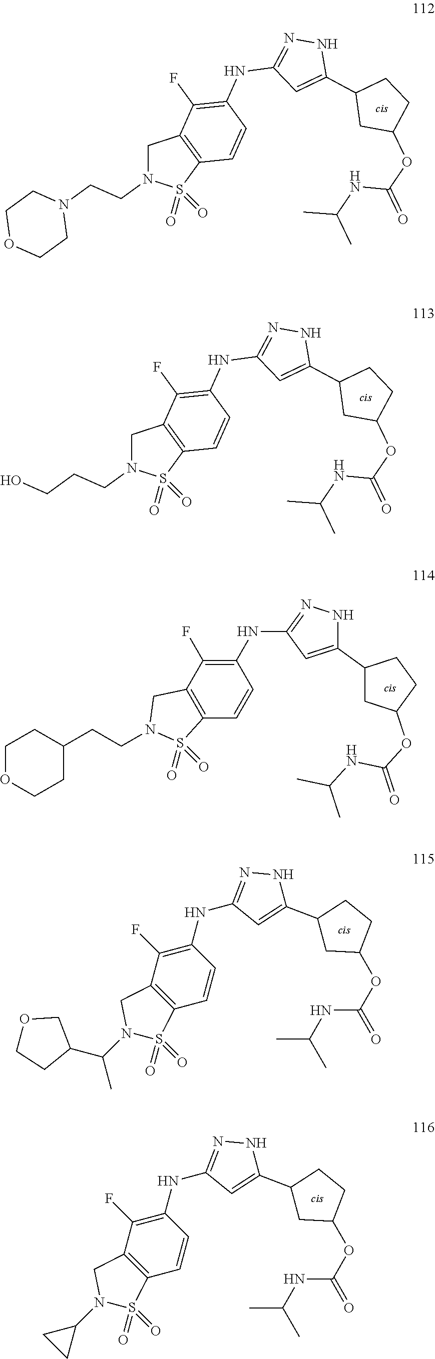 [SMILES: CC(C)NC(=O)OC1CCC(c2cc(Nc3ccc4c(c3F)CN(CCN3CCOCC3)S4(=O)=O)n[nH]2)C1 ; CC(C)NC(=O)OC1CCC(c2cc(Nc3ccc4c(c3F)CN(CCCO)S4(=O)=O)n[nH]2)C1 ; CC(C)NC(=O)OC1CCC(c2cc(Nc3ccc4c(c3F)CN(CCC3CCOCC3)S4(=O)=O)n[nH]2)C1 ; CC(C)NC(=O)OC1CCC(c2cc(Nc3ccc4c(c3F)CN(C(C)C3CCOC3)S4(=O)=O)n[nH]2)C1 ; CC(C)NC(=O)OC1CCC(c2cc(Nc3ccc4c(c3F)CN(C3CC3)S4(=O)=O)n[nH]2)C1] 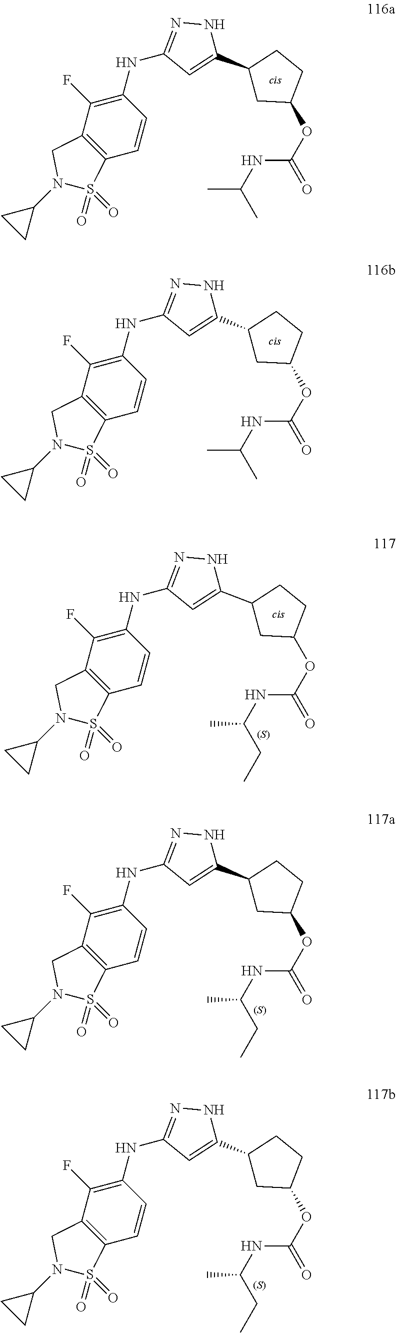 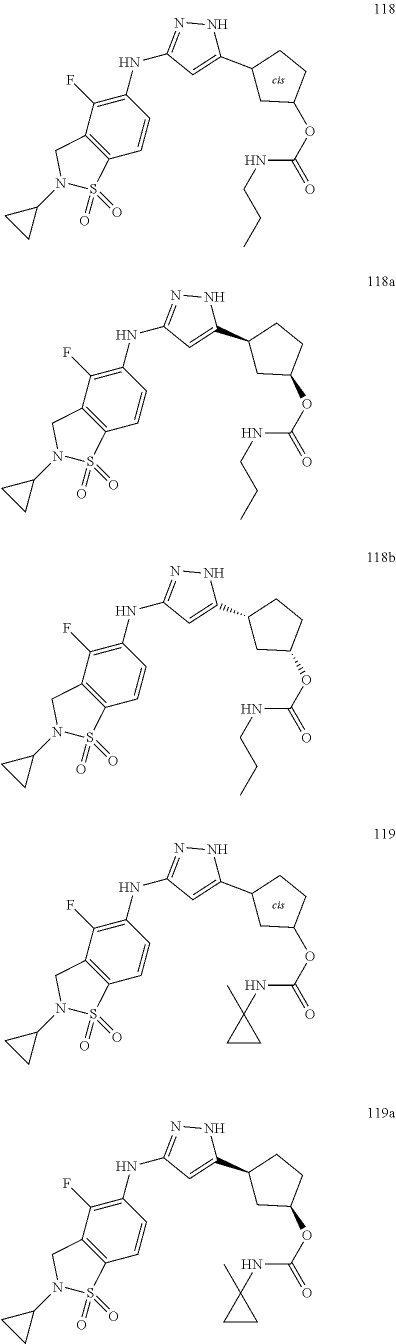 [SMILES: CCCNC(=O)OC1CCC(c2cc(Nc3ccc4c(c3F)CN(C3CC3)S4(=O)=O)n[nH]2)C1 ; CCCNC(=O)O[C@@H]1CC[C@H](c2cc(Nc3ccc4c(c3F)CN(C3CC3)S4(=O)=O)n[nH]2)C1 ; CCCNC(=O)O[C@H]1CC[C@@H](c2cc(Nc3ccc4c(c3F)CN(C3CC3)S4(=O)=O)n[nH]2)C1 ; CC1(NC(=O)OC2CCC(c3cc(Nc4ccc5c(c4F)CN(C4CC4)S5(=O)=O)n[nH]3)C2)CC1 ; CC1(NC(=O)O[C@@H]2CC[C@H](c3cc(Nc4ccc5c(c4F)CN(C4CC4)S5(=O)=O)n[nH]3)C2)CC1] 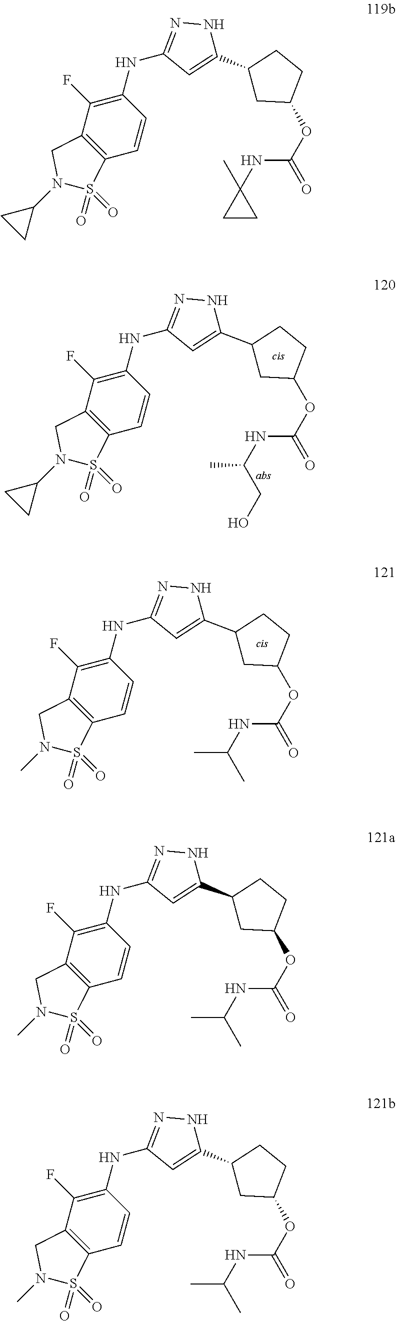 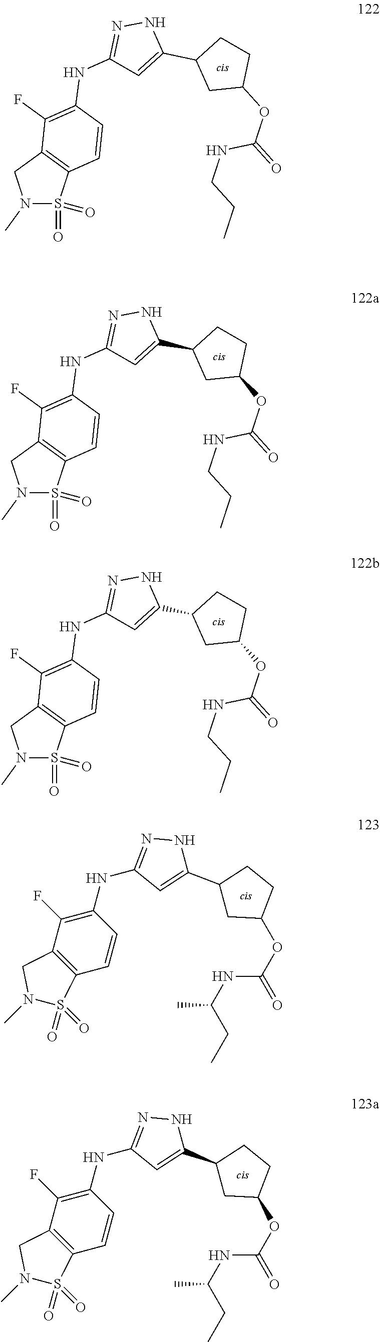 [SMILES: CCCNC(=O)OC1CCC(c2cc(Nc3ccc4c(c3F)CN(C)S4(=O)=O)n[nH]2)C1 ; CCCNC(=O)O[C@@H]1CC[C@H](c2cc(Nc3ccc4c(c3F)CN(C)S4(=O)=O)n[nH]2)C1 ; CCCNC(=O)O[C@H]1CC[C@@H](c2cc(Nc3ccc4c(c3F)CN(C)S4(=O)=O)n[nH]2)C1 ; CC[C@H](C)NC(=O)OC1CCC(c2cc(Nc3ccc4c(c3F)CN(C)S4(=O)=O)n[nH]2)C1 ; CC[C@H](C)NC(=O)O[C@@H]1CC[C@H](c2cc(Nc3ccc4c(c3F)CN(C)S4(=O)=O)n[nH]2)C1] 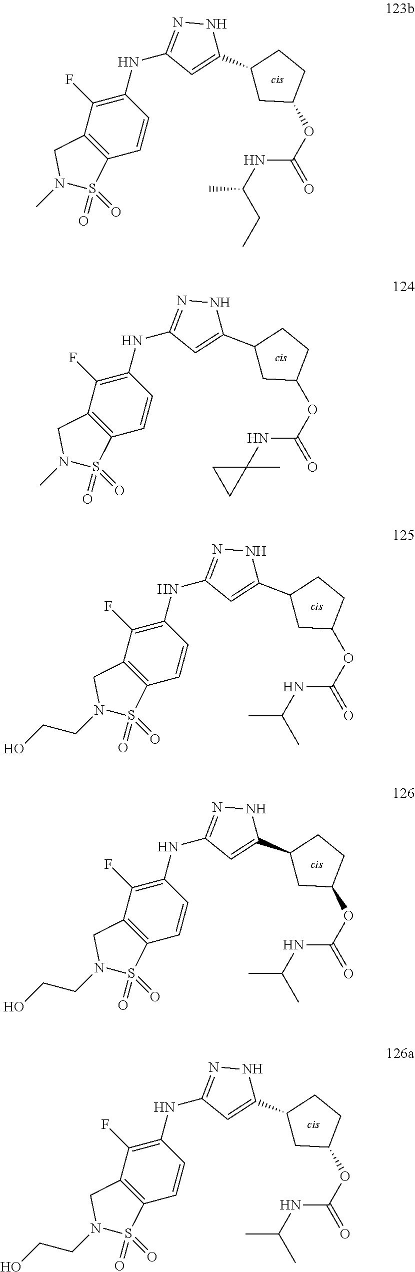 [SMILES: CC[C@H](C)NC(=O)O[C@H]1CC[C@@H](c2cc(Nc3ccc4c(c3F)CN(C)S4(=O)=O)n[nH]2)C1 ; CN1Cc2c(ccc(Nc3cc(C4CCC(OC(=O)NC5(C)CC5)C4)[nH]n3)c2F)S1(=O)=O ; CC(C)NC(=O)OC1CCC(c2cc(Nc3ccc4c(c3F)CN(CCO)S4(=O)=O)n[nH]2)C1 ; CC(C)NC(=O)O[C@@H]1CC[C@H](c2cc(Nc3ccc4c(c3F)CN(CCO)S4(=O)=O)n[nH]2)C1 ; CC(C)NC(=O)O[C@H]1CC[C@@H](c2cc(Nc3ccc4c(c3F)CN(CCO)S4(=O)=O)n[nH]2)C1] 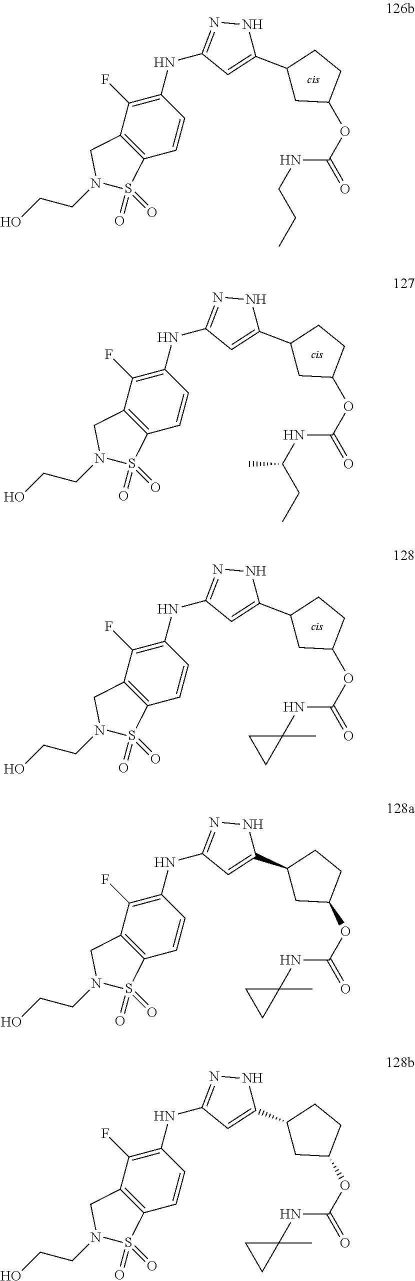 [SMILES: CCCNC(=O)OC1CCC(c2cc(Nc3ccc4c(c3F)CN(CCO)S4(=O)=O)n[nH]2)C1 ; CC[C@H](C)NC(=O)OC1CCC(c2cc(Nc3ccc4c(c3F)CN(CCO)S4(=O)=O)n[nH]2)C1 ; CC1(NC(=O)OC2CCC(c3cc(Nc4ccc5c(c4F)CN(CCO)S5(=O)=O)n[nH]3)C2)CC1 ; CC1(NC(=O)O[C@@H]2CC[C@H](c3cc(Nc4ccc5c(c4F)CN(CCO)S5(=O)=O)n[nH]3)C2)CC1 ; CC1(NC(=O)O[C@H]2CC[C@@H](c3cc(Nc4ccc5c(c4F)CN(CCO)S5(=O)=O)n[nH]3)C2)CC1] 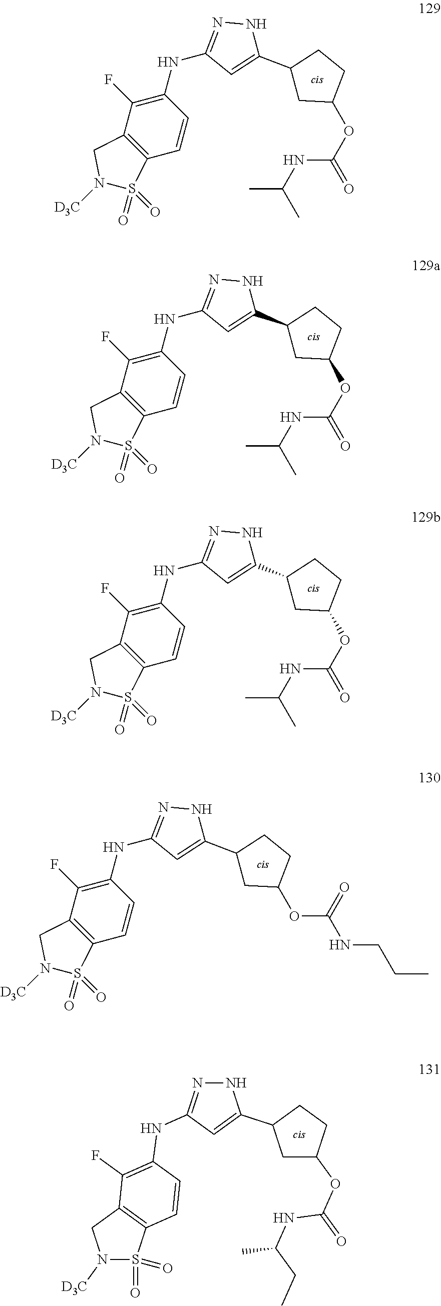 [SMILES: [2H]C([2H])([2H])N1Cc2c(ccc(Nc3cc(C4CCC(OC(=O)NC(C)C)C4)[nH]n3)c2F)S1(=O)=O ; [2H]C([2H])([2H])N1Cc2c(ccc(Nc3cc([C@H]4CC[C@@H](OC(=O)NC(C)C)C4)[nH]n3)c2F)S1(=O)=O ; [2H]C([2H])([2H])N1Cc2c(ccc(Nc3cc([C@@H]4CC[C@H](OC(=O)NC(C)C)C4)[nH]n3)c2F)S1(=O)=O ; [2H]C([2H])([2H])N1Cc2c(ccc(Nc3cc(C4CCC(OC(=O)NCCC)C4)[nH]n3)c2F)S1(=O)=O ; [2H]C([2H])([2H])N1Cc2c(ccc(Nc3cc(C4CCC(OC(=O)N[C@@H](C)CC)C4)[nH]n3)c2F)S1(=O)=O] 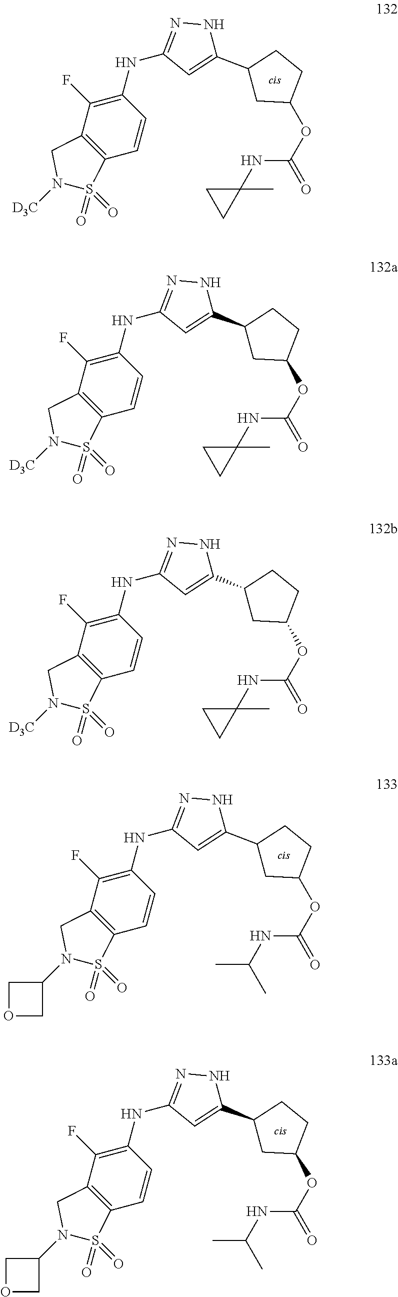 [SMILES: [2H]C([2H])([2H])N1Cc2c(ccc(Nc3cc(C4CCC(OC(=O)NC5(C)CC5)C4)[nH]n3)c2F)S1(=O)=O ; [2H]C([2H])([2H])N1Cc2c(ccc(Nc3cc([C@H]4CC[C@@H](OC(=O)NC5(C)CC5)C4)[nH]n3)c2F)S1(=O)=O ; [2H]C([2H])([2H])N1Cc2c(ccc(Nc3cc([C@@H]4CC[C@H](OC(=O)NC5(C)CC5)C4)[nH]n3)c2F)S1(=O)=O ; CC(C)NC(=O)OC1CCC(c2cc(Nc3ccc4c(c3F)CN(C3COC3)S4(=O)=O)n[nH]2)C1 ; CC(C)NC(=O)O[C@@H]1CC[C@H](c2cc(Nc3ccc4c(c3F)CN(C3COC3)S4(=O)=O)n[nH]2)C1] 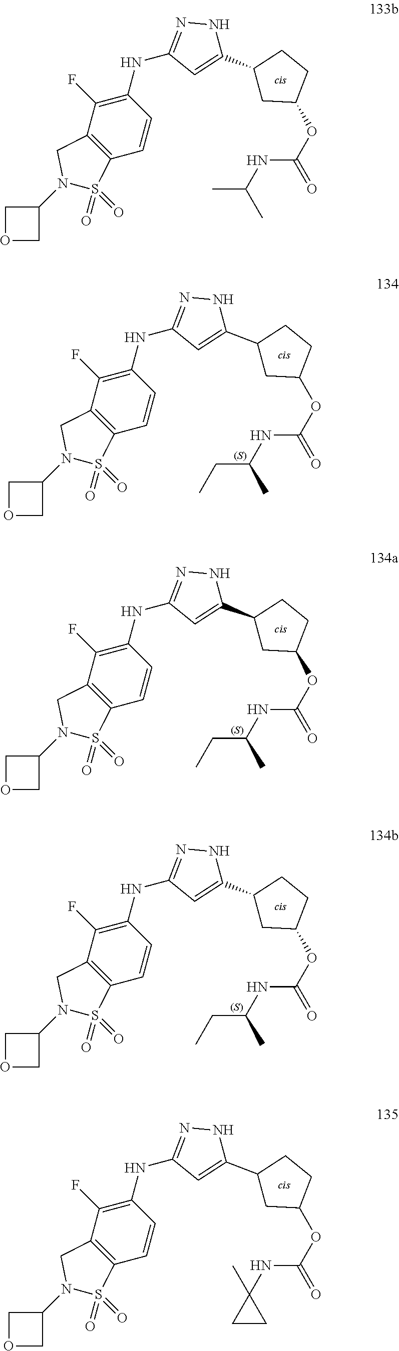 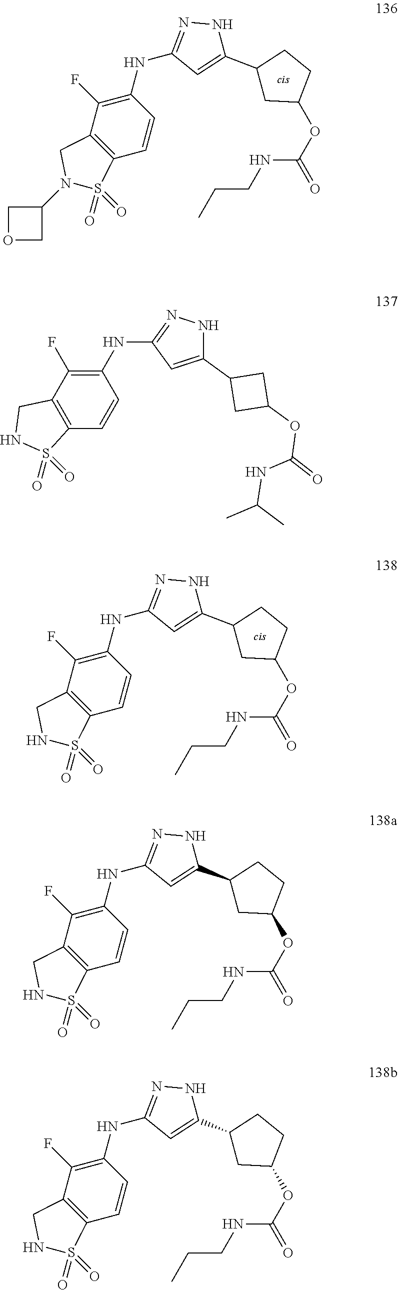 [SMILES: CCCNC(=O)OC1CCC(c2cc(Nc3ccc4c(c3F)CN(C3COC3)S4(=O)=O)n[nH]2)C1 ; CC(C)NC(=O)OC1CC(c2cc(Nc3ccc4c(c3F)CNS4(=O)=O)n[nH]2)C1 ; CCCNC(=O)OC1CCC(c2cc(Nc3ccc4c(c3F)CNS4(=O)=O)n[nH]2)C1 ; CCCNC(=O)O[C@@H]1CC[C@H](c2cc(Nc3ccc4c(c3F)CNS4(=O)=O)n[nH]2)C1 ; CCCNC(=O)O[C@H]1CC[C@@H](c2cc(Nc3ccc4c(c3F)CNS4(=O)=O)n[nH]2)C1] 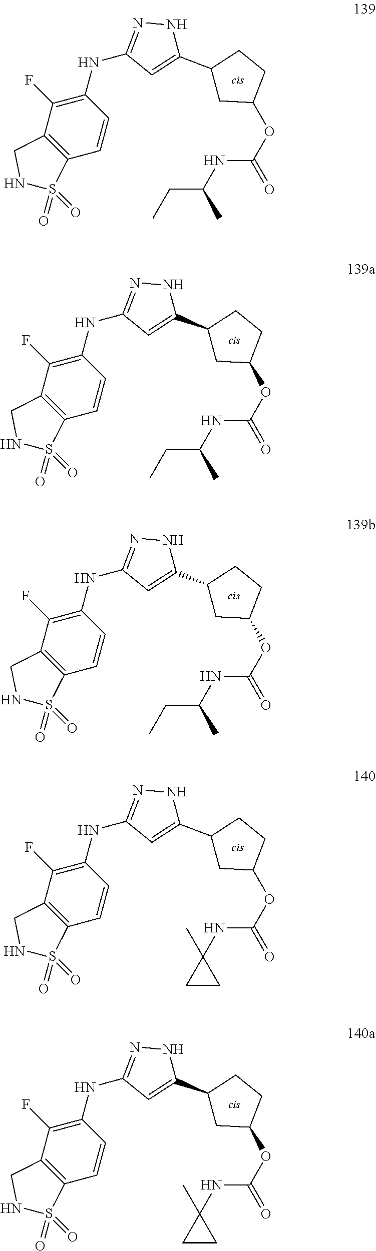 [SMILES: CC[C@H](C)NC(=O)OC1CCC(c2cc(Nc3ccc4c(c3F)CNS4(=O)=O)n[nH]2)C1 ; CC[C@H](C)NC(=O)O[C@@H]1CC[C@H](c2cc(Nc3ccc4c(c3F)CNS4(=O)=O)n[nH]2)C1 ; CC[C@H](C)NC(=O)O[C@H]1CC[C@@H](c2cc(Nc3ccc4c(c3F)CNS4(=O)=O)n[nH]2)C1 ; CC1(NC(=O)OC2CCC(c3cc(Nc4ccc5c(c4F)CNS5(=O)=O)n[nH]3)C2)CC1 ; CC1(NC(=O)O[C@@H]2CC[C@H](c3cc(Nc4ccc5c(c4F)CNS5(=O)=O)n[nH]3)C2)CC1] 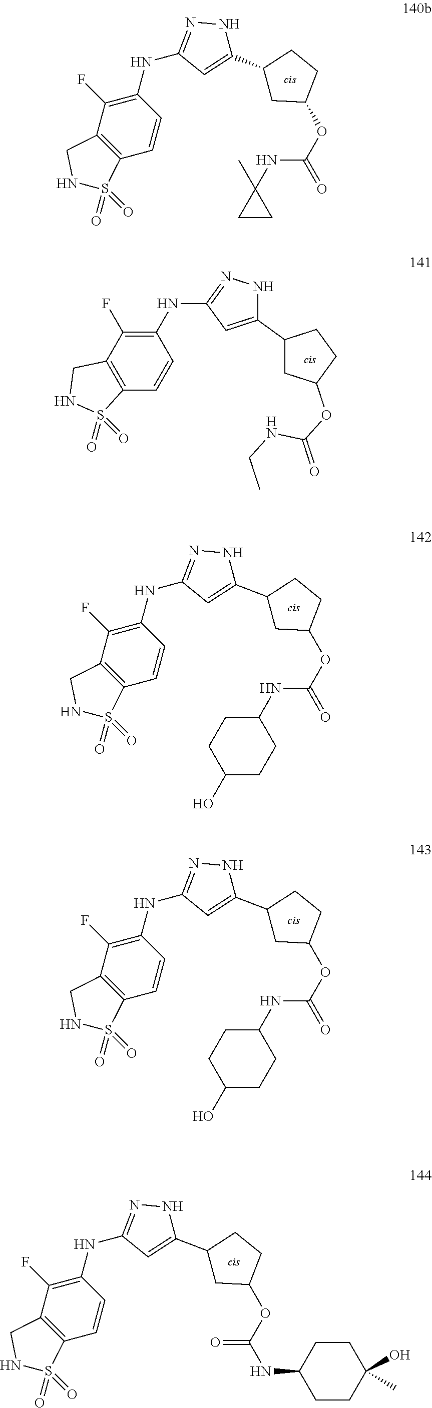 [SMILES: CC1(NC(=O)O[C@H]2CC[C@@H](c3cc(Nc4ccc5c(c4F)CNS5(=O)=O)n[nH]3)C2)CC1 ; CCNC(=O)OC1CCC(c2cc(Nc3ccc4c(c3F)CNS4(=O)=O)n[nH]2)C1 ; O=C(NC1CCC(O)CC1)OC1CCC(c2cc(Nc3ccc4c(c3F)CNS4(=O)=O)n[nH]2)C1 ; O=C(NC1CCC(O)CC1)OC1CCC(c2cc(Nc3ccc4c(c3F)CNS4(=O)=O)n[nH]2)C1 ; C[C@]1(O)CC[C@@H](NC(=O)OC2CCC(c3cc(Nc4ccc5c(c4F)CNS5(=O)=O)n[nH]3)C2)CC1] 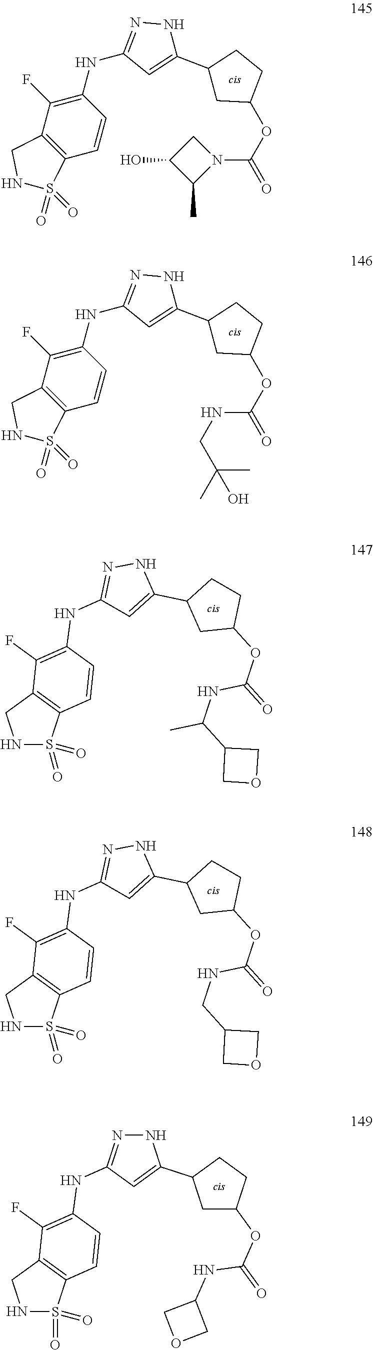 [SMILES: C[C@H]1[C@H](O)CN1C(=O)OC1CCC(c2cc(Nc3ccc4c(c3F)CNS4(=O)=O)n[nH]2)C1 ; CC(C)(O)CNC(=O)OC1CCC(c2cc(Nc3ccc4c(c3F)CNS4(=O)=O)n[nH]2)C1 ; CC(NC(=O)OC1CCC(c2cc(Nc3ccc4c(c3F)CNS4(=O)=O)n[nH]2)C1)C1COC1 ; O=C(NCC1COC1)OC1CCC(c2cc(Nc3ccc4c(c3F)CNS4(=O)=O)n[nH]2)C1 ; O=C(NC1COC1)OC1CCC(c2cc(Nc3ccc4c(c3F)CNS4(=O)=O)n[nH]2)C1] 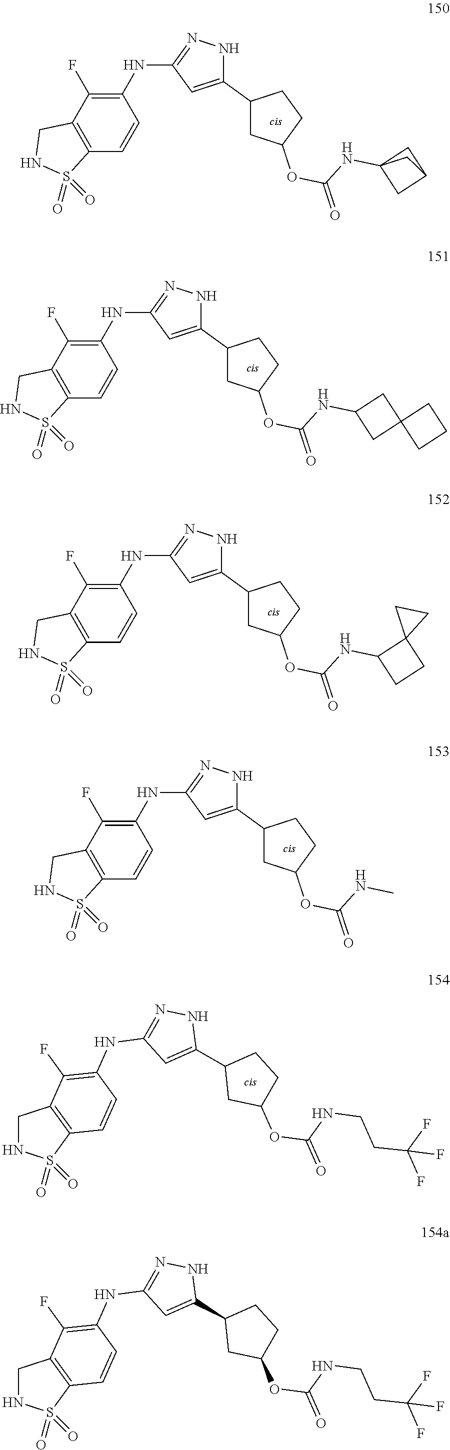 [SMILES: O=C(NC12CC(C1)C2)OC1CCC(c2cc(Nc3ccc4c(c3F)CNS4(=O)=O)n[nH]2)C1 ; O=C(NC1CC2(CCC2)C1)OC1CCC(c2cc(Nc3ccc4c(c3F)CNS4(=O)=O)n[nH]2)C1 ; O=C(NC1CCC12CC2)OC1CCC(c2cc(Nc3ccc4c(c3F)CNS4(=O)=O)n[nH]2)C1 ; CNC(=O)OC1CCC(c2cc(Nc3ccc4c(c3F)CNS4(=O)=O)n[nH]2)C1 ; O=C(NCCC(F)(F)F)OC1CCC(c2cc(Nc3ccc4c(c3F)CNS4(=O)=O)n[nH]2)C1 ; O=C(NCCC(F)(F)F)O[C@@H]1CC[C@H](c2cc(Nc3ccc4c(c3F)CNS4(=O)=O)n[nH]2)C1] 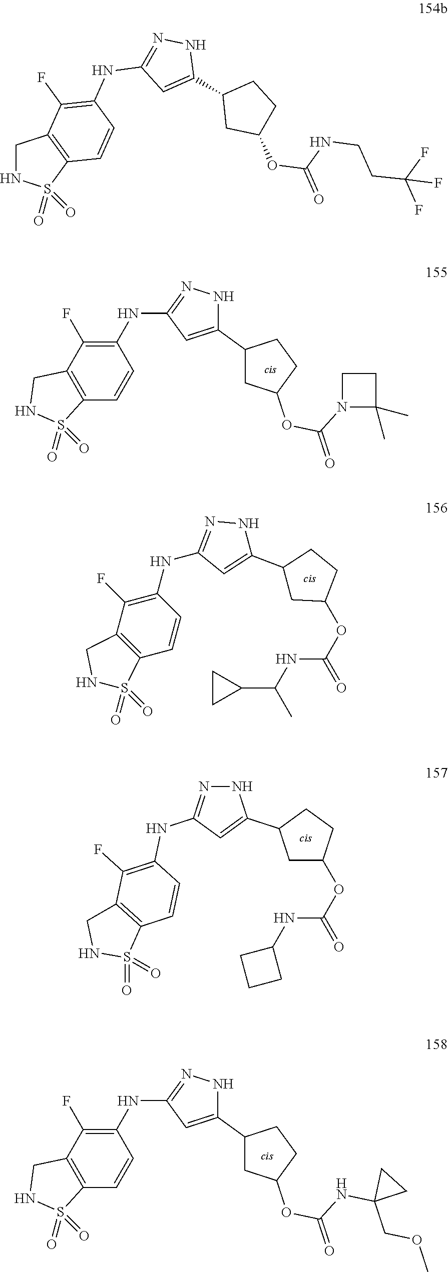 [SMILES: O=C(NCCC(F)(F)F)O[C@H]1CC[C@@H](c2cc(Nc3ccc4c(c3F)CNS4(=O)=O)n[nH]2)C1 ; CC1(C)CCN1C(=O)OC1CCC(c2cc(Nc3ccc4c(c3F)CNS4(=O)=O)n[nH]2)C1 ; CC(NC(=O)OC1CCC(c2cc(Nc3ccc4c(c3F)CNS4(=O)=O)n[nH]2)C1)C1CC1 ; O=C(NC1CCC1)OC1CCC(c2cc(Nc3ccc4c(c3F)CNS4(=O)=O)n[nH]2)C1 ; COCC1(NC(=O)OC2CCC(c3cc(Nc4ccc5c(c4F)CNS5(=O)=O)n[nH]3)C2)CC1] 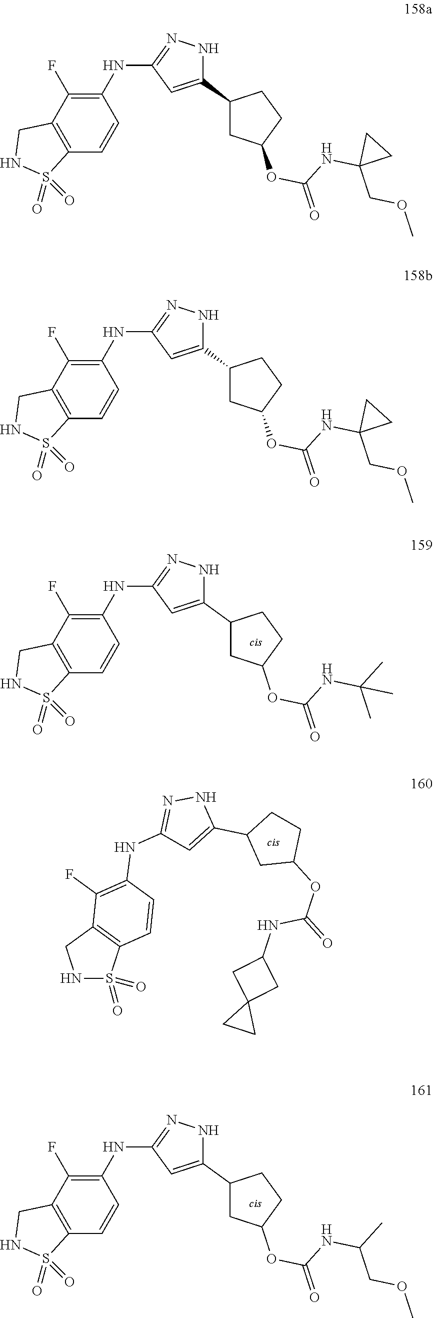 [SMILES: COCC1(NC(=O)O[C@@H]2CC[C@H](c3cc(Nc4ccc5c(c4F)CNS5(=O)=O)n[nH]3)C2)CC1 ; COCC1(NC(=O)O[C@H]2CC[C@@H](c3cc(Nc4ccc5c(c4F)CNS5(=O)=O)n[nH]3)C2)CC1 ; CC(C)(C)NC(=O)OC1CCC(c2cc(Nc3ccc4c(c3F)CNS4(=O)=O)n[nH]2)C1 ; O=C(NC1CC2(CC2)C1)OC1CCC(c2cc(Nc3ccc4c(c3F)CNS4(=O)=O)n[nH]2)C1 ; COCC(C)NC(=O)OC1CCC(c2cc(Nc3ccc4c(c3F)CNS4(=O)=O)n[nH]2)C1] 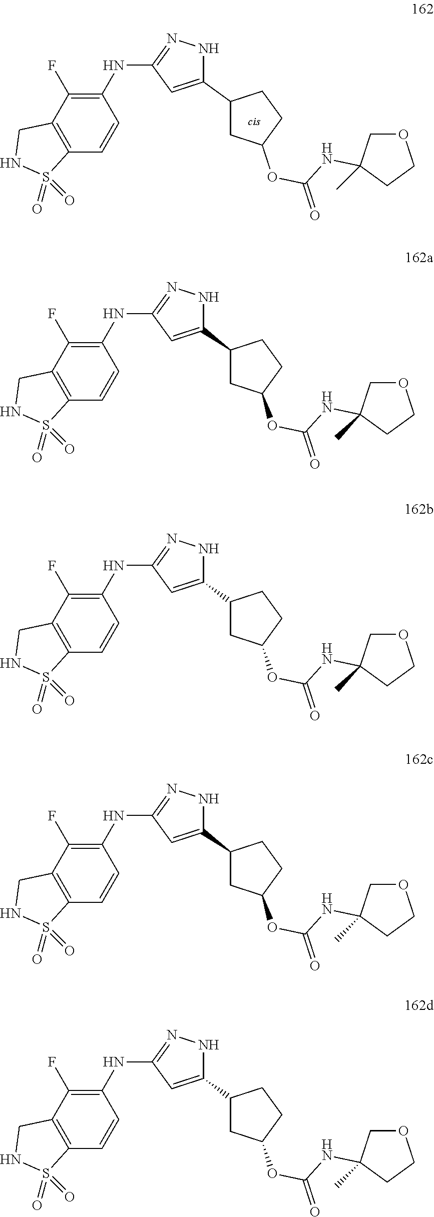 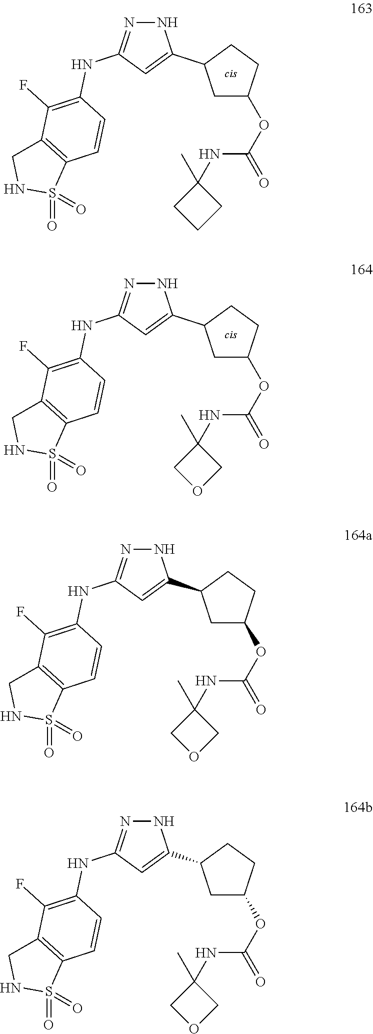 [SMILES: CC1(NC(=O)OC2CCC(c3cc(Nc4ccc5c(c4F)CNS5(=O)=O)n[nH]3)C2)CCC1 ; CC1(NC(=O)OC2CCC(c3cc(Nc4ccc5c(c4F)CNS5(=O)=O)n[nH]3)C2)COC1 ; CC1(NC(=O)O[C@@H]2CC[C@H](c3cc(Nc4ccc5c(c4F)CNS5(=O)=O)n[nH]3)C2)COC1 ; CC1(NC(=O)O[C@H]2CC[C@@H](c3cc(Nc4ccc5c(c4F)CNS5(=O)=O)n[nH]3)C2)COC1] 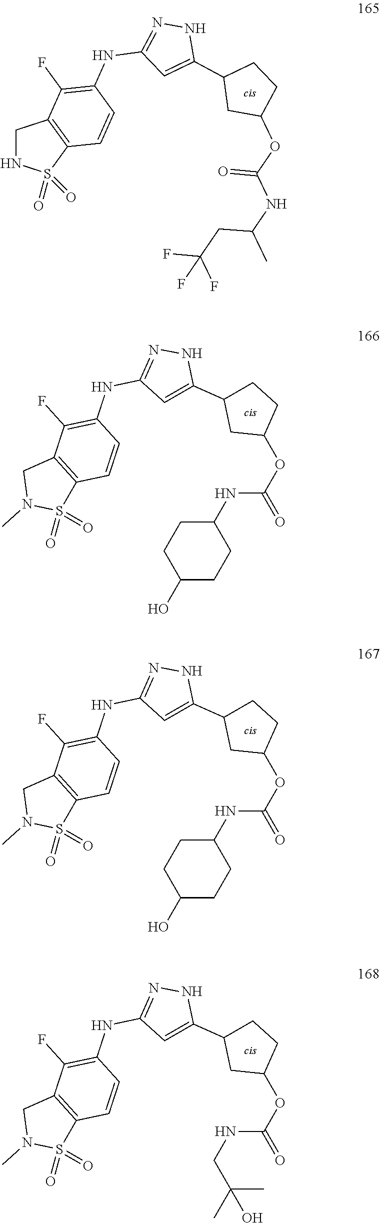 [SMILES: CC(CC(F)(F)F)NC(=O)OC1CCC(c2cc(Nc3ccc4c(c3F)CNS4(=O)=O)n[nH]2)C1 ; CN1Cc2c(ccc(Nc3cc(C4CCC(OC(=O)NC5CCC(O)CC5)C4)[nH]n3)c2F)S1(=O)=O ; CN1Cc2c(ccc(Nc3cc(C4CCC(OC(=O)NC5CCC(O)CC5)C4)[nH]n3)c2F)S1(=O)=O ; CN1Cc2c(ccc(Nc3cc(C4CCC(OC(=O)NCC(C)(C)O)C4)[nH]n3)c2F)S1(=O)=O] 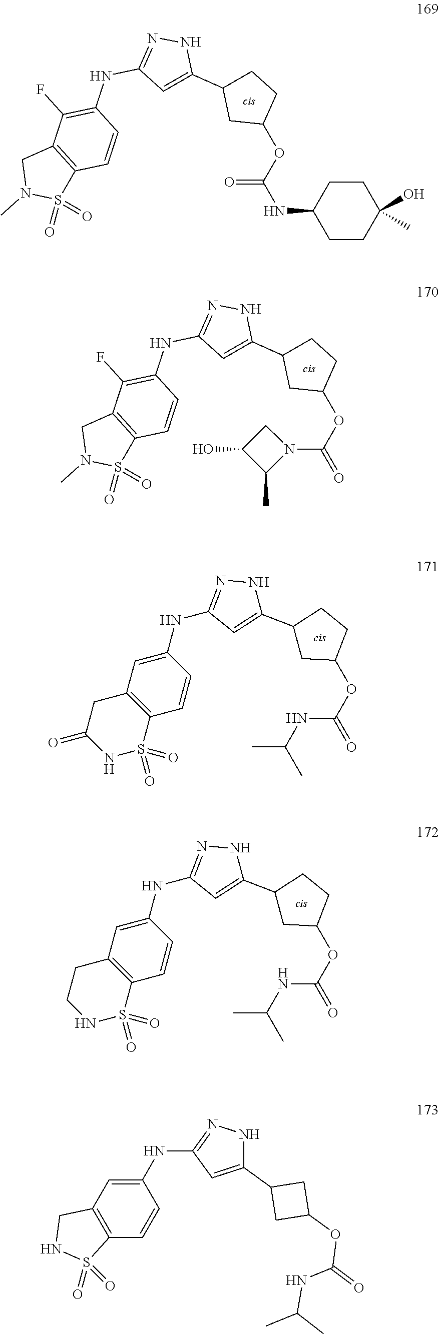 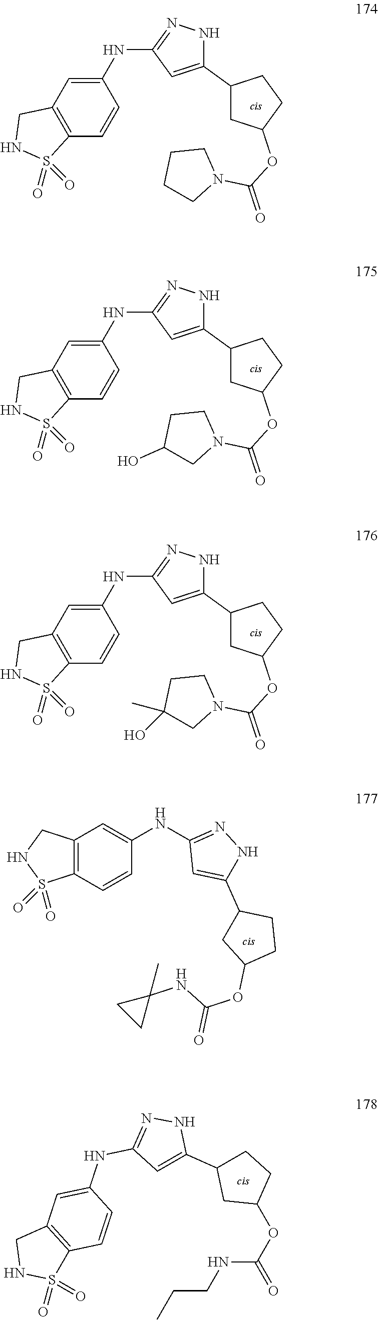 [SMILES: O=C(OC1CCC(c2cc(Nc3ccc4c(c3)CNS4(=O)=O)n[nH]2)C1)N1CCCC1 ; O=C(OC1CCC(c2cc(Nc3ccc4c(c3)CNS4(=O)=O)n[nH]2)C1)N1CCC(O)C1 ; CC1(O)CCN(C(=O)OC2CCC(c3cc(Nc4ccc5c(c4)CNS5(=O)=O)n[nH]3)C2)C1 ; CC1(NC(=O)OC2CCC(c3cc(Nc4ccc5c(c4)CNS5(=O)=O)n[nH]3)C2)CC1 ; CCCNC(=O)OC1CCC(c2cc(Nc3ccc4c(c3)CNS4(=O)=O)n[nH]2)C1] 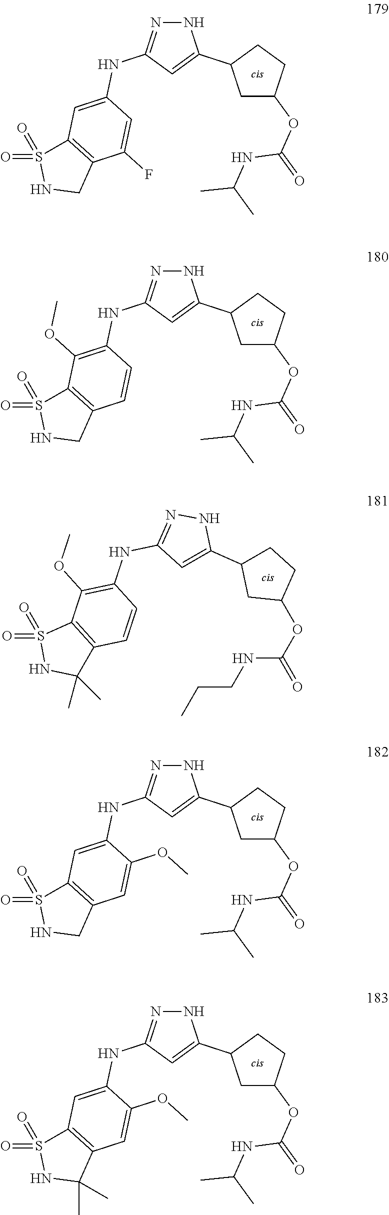 [SMILES: CC(C)NC(=O)OC1CCC(c2cc(Nc3cc(F)c4c(c3)S(=O)(=O)NC4)n[nH]2)C1 ; COc1c(Nc2cc(C3CCC(OC(=O)NC(C)C)C3)[nH]n2)ccc2c1S(=O)(=O)NC2 ; CCCNC(=O)OC1CCC(c2cc(Nc3ccc4c(c3OC)S(=O)(=O)NC4(C)C)n[nH]2)C1 ; COc1cc2c(cc1Nc1cc(C3CCC(OC(=O)NC(C)C)C3)[nH]n1)S(=O)(=O)NC2 ; COc1cc2c(cc1Nc1cc(C3CCC(OC(=O)NC(C)C)C3)[nH]n1)S(=O)(=O)NC2(C)C] 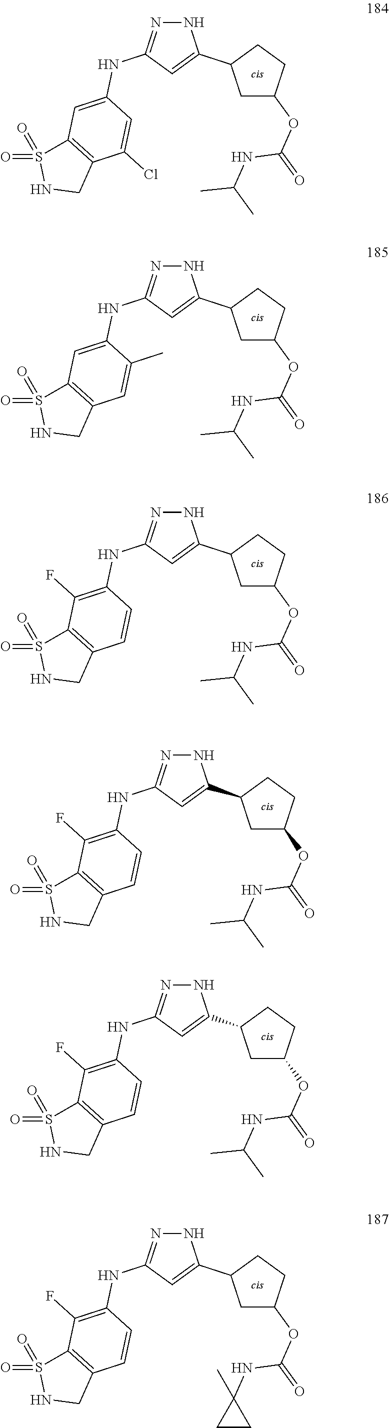 [SMILES: CC(C)NC(=O)OC1CCC(c2cc(Nc3cc(Cl)c4c(c3)S(=O)(=O)NC4)n[nH]2)C1 ; Cc1cc2c(cc1Nc1cc(C3CCC(OC(=O)NC(C)C)C3)[nH]n1)S(=O)(=O)NC2 ; CC(C)NC(=O)OC1CCC(c2cc(Nc3ccc4c(c3F)S(=O)(=O)NC4)n[nH]2)C1 ; CC(C)NC(=O)O[C@@H]1CC[C@H](c2cc(Nc3ccc4c(c3F)S(=O)(=O)NC4)n[nH]2)C1 ; CC(C)NC(=O)O[C@H]1CC[C@@H](c2cc(Nc3ccc4c(c3F)S(=O)(=O)NC4)n[nH]2)C1 ; CC1(NC(=O)OC2CCC(c3cc(Nc4ccc5c(c4F)S(=O)(=O)NC5)n[nH]3)C2)CC1] 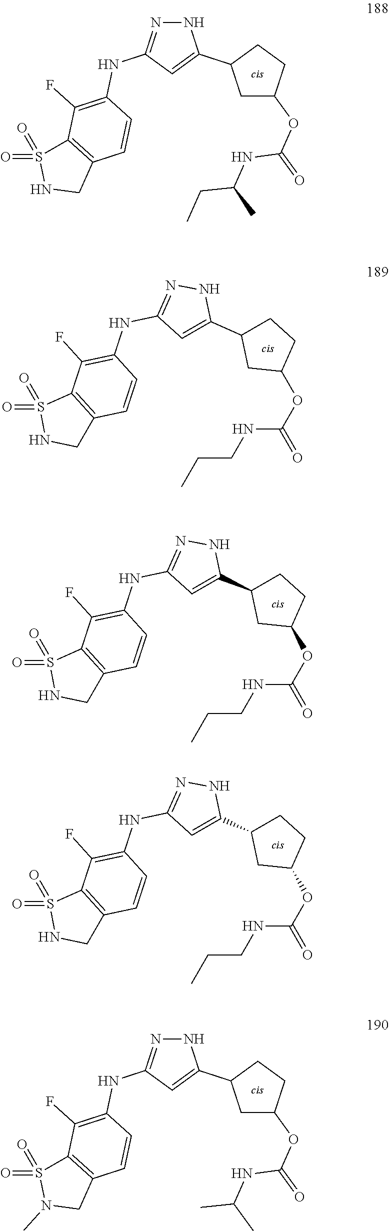 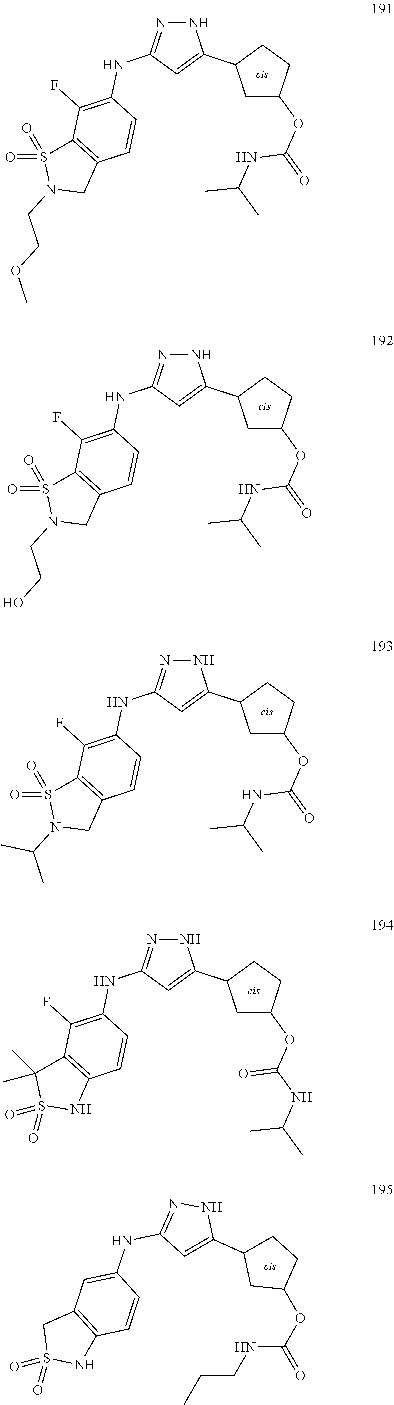 [SMILES: COCCN1Cc2ccc(Nc3cc(C4CCC(OC(=O)NC(C)C)C4)[nH]n3)c(F)c2S1(=O)=O ; CC(C)NC(=O)OC1CCC(c2cc(Nc3ccc4c(c3F)S(=O)(=O)N(CCO)C4)n[nH]2)C1 ; CC(C)NC(=O)OC1CCC(c2cc(Nc3ccc4c(c3F)S(=O)(=O)N(C(C)C)C4)n[nH]2)C1 ; CC(C)NC(=O)OC1CCC(c2cc(Nc3ccc4c(c3F)C(C)(C)S(=O)(=O)N4)n[nH]2)C1 ; CCCNC(=O)OC1CCC(c2cc(Nc3ccc4c(c3)CS(=O)(=O)N4)n[nH]2)C1] 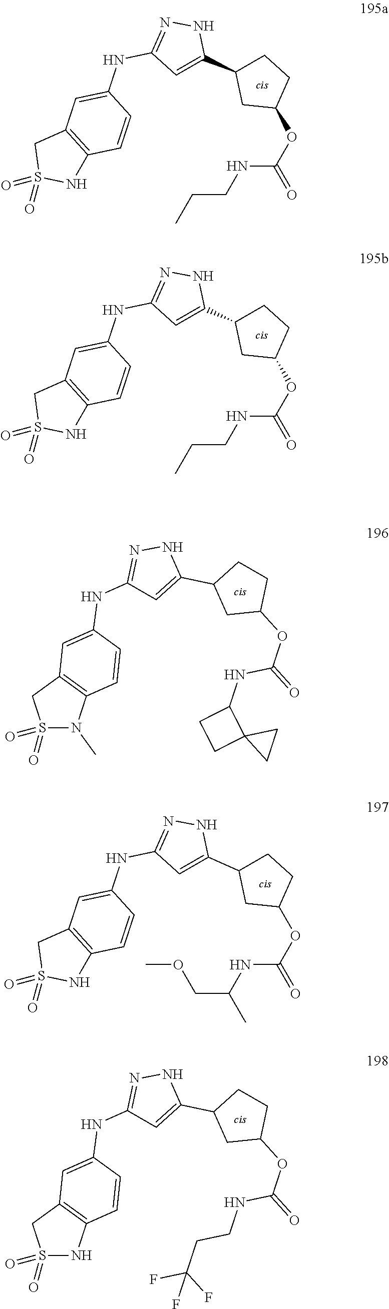 [SMILES: CCCNC(=O)O[C@@H]1CC[C@H](c2cc(Nc3ccc4c(c3)CS(=O)(=O)N4)n[nH]2)C1 ; CCCNC(=O)O[C@H]1CC[C@@H](c2cc(Nc3ccc4c(c3)CS(=O)(=O)N4)n[nH]2)C1 ; CN1c2ccc(Nc3cc(C4CCC(OC(=O)NC5CCC56CC6)C4)[nH]n3)cc2CS1(=O)=O ; COCC(C)NC(=O)OC1CCC(c2cc(Nc3ccc4c(c3)CS(=O)(=O)N4)n[nH]2)C1 ; O=C(NCCC(F)(F)F)OC1CCC(c2cc(Nc3ccc4c(c3)CS(=O)(=O)N4)n[nH]2)C1] 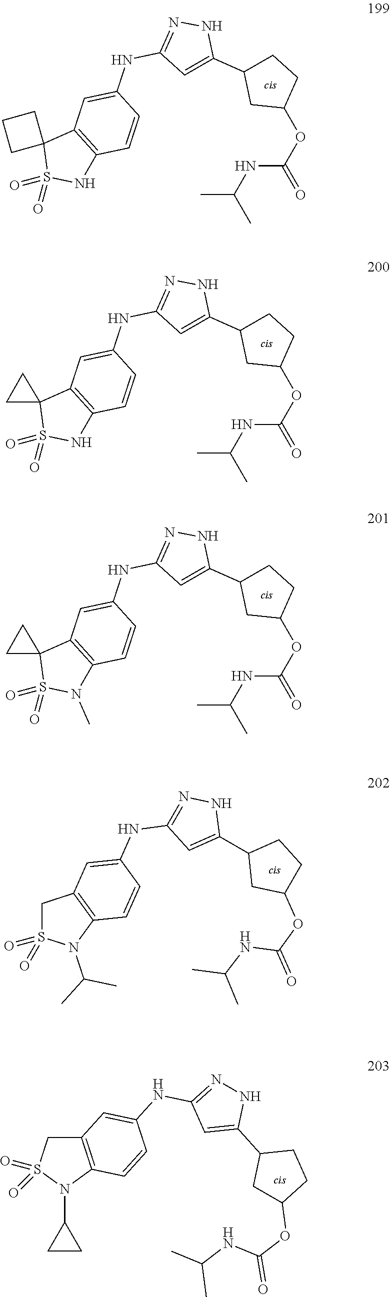 [SMILES: CC(C)NC(=O)OC1CCC(c2cc(Nc3ccc4c(c3)C3(CCC3)S(=O)(=O)N4)n[nH]2)C1 ; CC(C)NC(=O)OC1CCC(c2cc(Nc3ccc4c(c3)C3(CC3)S(=O)(=O)N4)n[nH]2)C1 ; CC(C)NC(=O)OC1CCC(c2cc(Nc3ccc4c(c3)C3(CC3)S(=O)(=O)N4C)n[nH]2)C1 ; CC(C)NC(=O)OC1CCC(c2cc(Nc3ccc4c(c3)CS(=O)(=O)N4C(C)C)n[nH]2)C1 ; CC(C)NC(=O)OC1CCC(c2cc(Nc3ccc4c(c3)CS(=O)(=O)N4C3CC3)n[nH]2)C1] 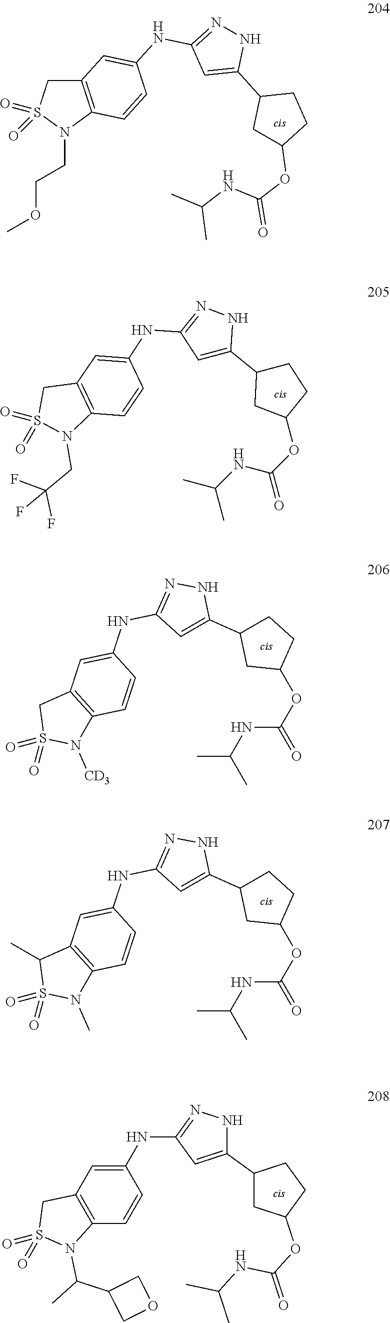 [SMILES: COCCN1c2ccc(Nc3cc(C4CCC(OC(=O)NC(C)C)C4)[nH]n3)cc2CS1(=O)=O ; CC(C)NC(=O)OC1CCC(c2cc(Nc3ccc4c(c3)CS(=O)(=O)N4CC(F)(F)F)n[nH]2)C1 ; [2H]C([2H])([2H])N1c2ccc(Nc3cc(C4CCC(OC(=O)NC(C)C)C4)[nH]n3)cc2CS1(=O)=O ; CC(C)NC(=O)OC1CCC(c2cc(Nc3ccc4c(c3)C(C)S(=O)(=O)N4C)n[nH]2)C1 ; CC(C)NC(=O)OC1CCC(c2cc(Nc3ccc4c(c3)CS(=O)(=O)N4C(C)C3COC3)n[nH]2)C1] 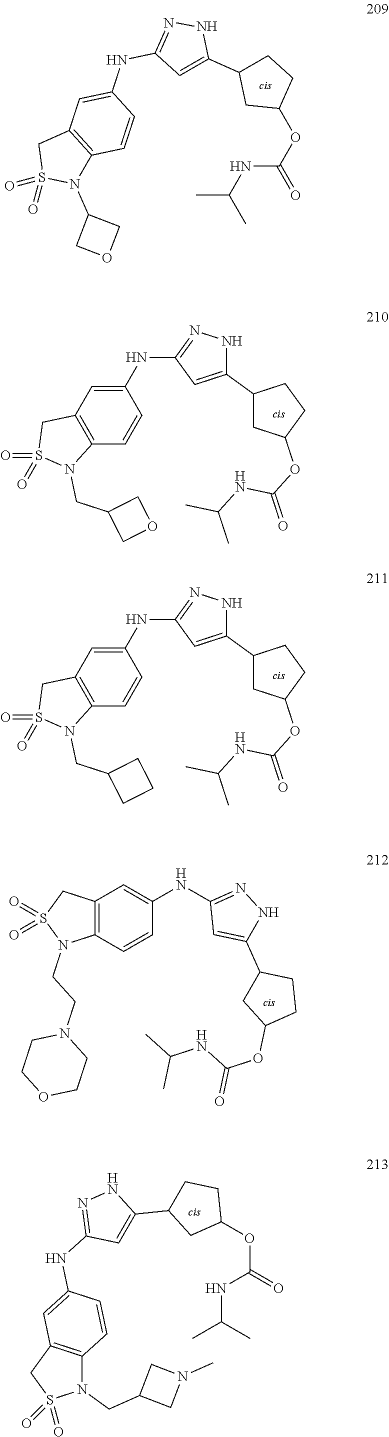 [SMILES: CC(C)NC(=O)OC1CCC(c2cc(Nc3ccc4c(c3)CS(=O)(=O)N4C3COC3)n[nH]2)C1 ; CC(C)NC(=O)OC1CCC(c2cc(Nc3ccc4c(c3)CS(=O)(=O)N4CC3COC3)n[nH]2)C1 ; CC(C)NC(=O)OC1CCC(c2cc(Nc3ccc4c(c3)CS(=O)(=O)N4CC3CCC3)n[nH]2)C1 ; CC(C)NC(=O)OC1CCC(c2cc(Nc3ccc4c(c3)CS(=O)(=O)N4CCN3CCOCC3)n[nH]2)C1 ; CC(C)NC(=O)OC1CCC(c2cc(Nc3ccc4c(c3)CS(=O)(=O)N4CC3CN(C)C3)n[nH]2)C1] 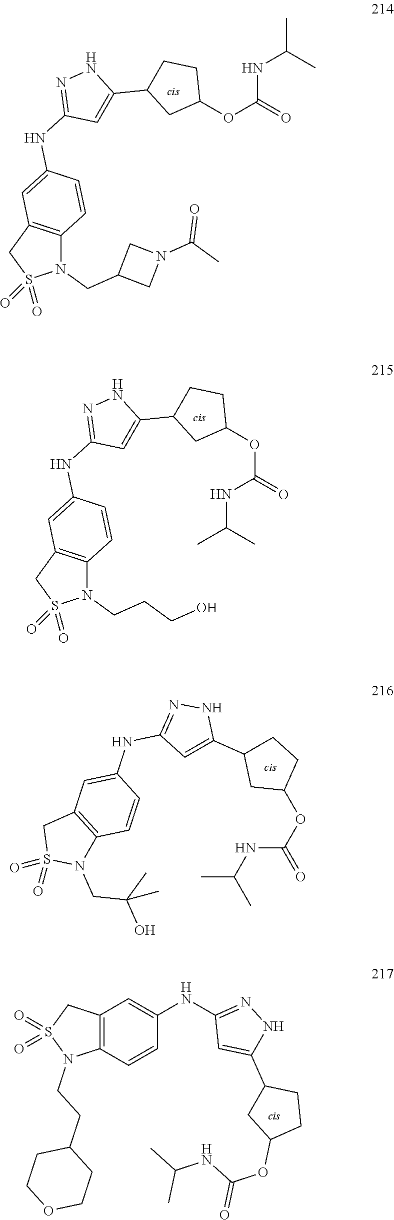 [SMILES: CC(=O)N1CC(CN2c3ccc(Nc4cc(C5CCC(OC(=O)NC(C)C)C5)[nH]n4)cc3CS2(=O)=O)C1 ; CC(C)NC(=O)OC1CCC(c2cc(Nc3ccc4c(c3)CS(=O)(=O)N4CCCO)n[nH]2)C1 ; CC(C)NC(=O)OC1CCC(c2cc(Nc3ccc4c(c3)CS(=O)(=O)N4CC(C)(C)O)n[nH]2)C1 ; CC(C)NC(=O)OC1CCC(c2cc(Nc3ccc4c(c3)CS(=O)(=O)N4CCC3CCOCC3)n[nH]2)C1] 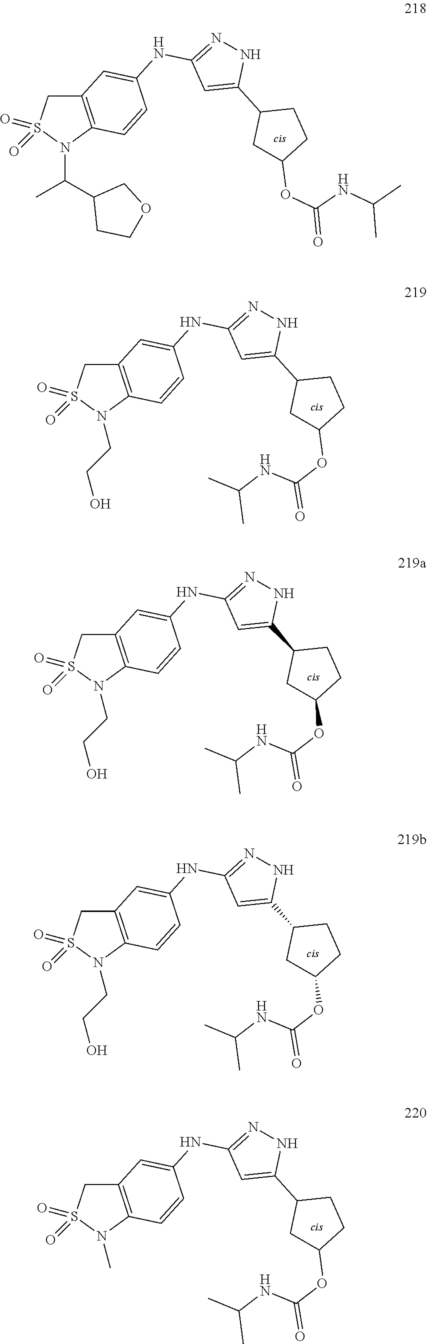 [SMILES: CC(C)NC(=O)OC1CCC(c2cc(Nc3ccc4c(c3)CS(=O)(=O)N4C(C)C3CCOC3)n[nH]2)C1 ; CC(C)NC(=O)OC1CCC(c2cc(Nc3ccc4c(c3)CS(=O)(=O)N4CCO)n[nH]2)C1 ; CC(C)NC(=O)O[C@@H]1CC[C@H](c2cc(Nc3ccc4c(c3)CS(=O)(=O)N4CCO)n[nH]2)C1 ; CC(C)NC(=O)O[C@H]1CC[C@@H](c2cc(Nc3ccc4c(c3)CS(=O)(=O)N4CCO)n[nH]2)C1 ; CC(C)NC(=O)OC1CCC(c2cc(Nc3ccc4c(c3)CS(=O)(=O)N4C)n[nH]2)C1] 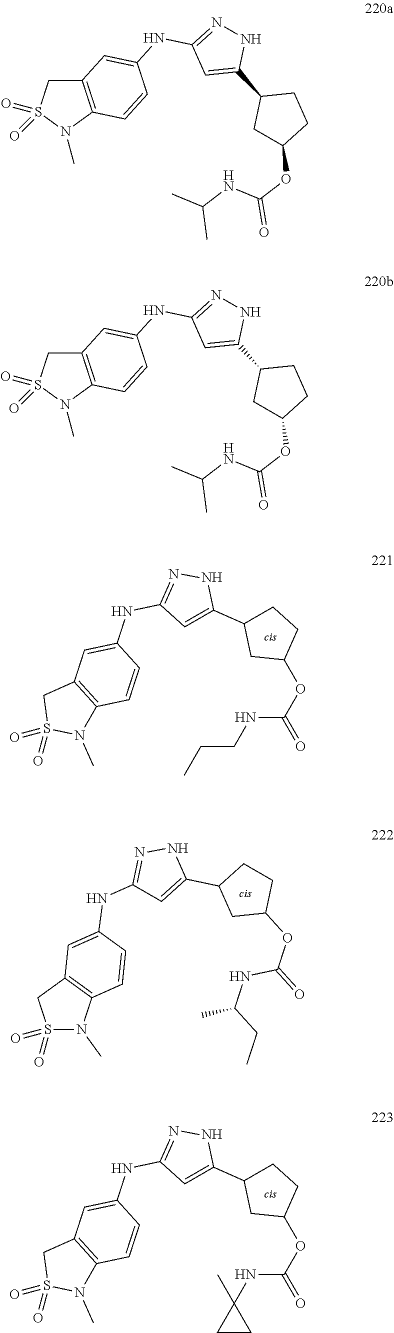 [SMILES: CC(C)NC(=O)O[C@@H]1CC[C@H](c2cc(Nc3ccc4c(c3)CS(=O)(=O)N4C)n[nH]2)C1 ; CC(C)NC(=O)O[C@H]1CC[C@@H](c2cc(Nc3ccc4c(c3)CS(=O)(=O)N4C)n[nH]2)C1 ; CCCNC(=O)OC1CCC(c2cc(Nc3ccc4c(c3)CS(=O)(=O)N4C)n[nH]2)C1 ; CC[C@H](C)NC(=O)OC1CCC(c2cc(Nc3ccc4c(c3)CS(=O)(=O)N4C)n[nH]2)C1 ; CN1c2ccc(Nc3cc(C4CCC(OC(=O)NC5(C)CC5)C4)[nH]n3)cc2CS1(=O)=O] 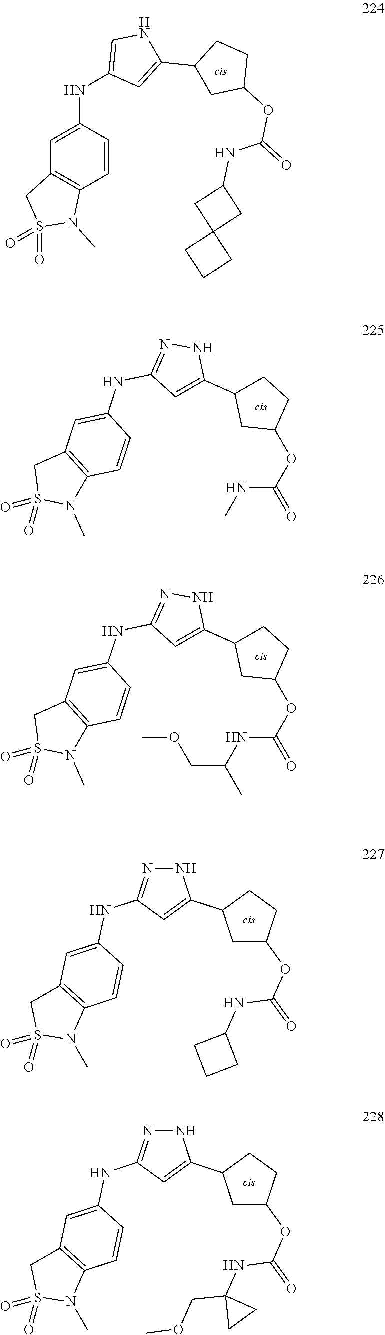 [SMILES: CN1c2ccc(Nc3c[nH]c(C4CCC(OC(=O)NC5CC6(CCC6)C5)C4)c3)cc2CS1(=O)=O ; CNC(=O)OC1CCC(c2cc(Nc3ccc4c(c3)CS(=O)(=O)N4C)n[nH]2)C1 ; COCC(C)NC(=O)OC1CCC(c2cc(Nc3ccc4c(c3)CS(=O)(=O)N4C)n[nH]2)C1 ; CN1c2ccc(Nc3cc(C4CCC(OC(=O)NC5CCC5)C4)[nH]n3)cc2CS1(=O)=O ; COCC1(NC(=O)OC2CCC(c3cc(Nc4ccc5c(c4)CS(=O)(=O)N5C)n[nH]3)C2)CC1] 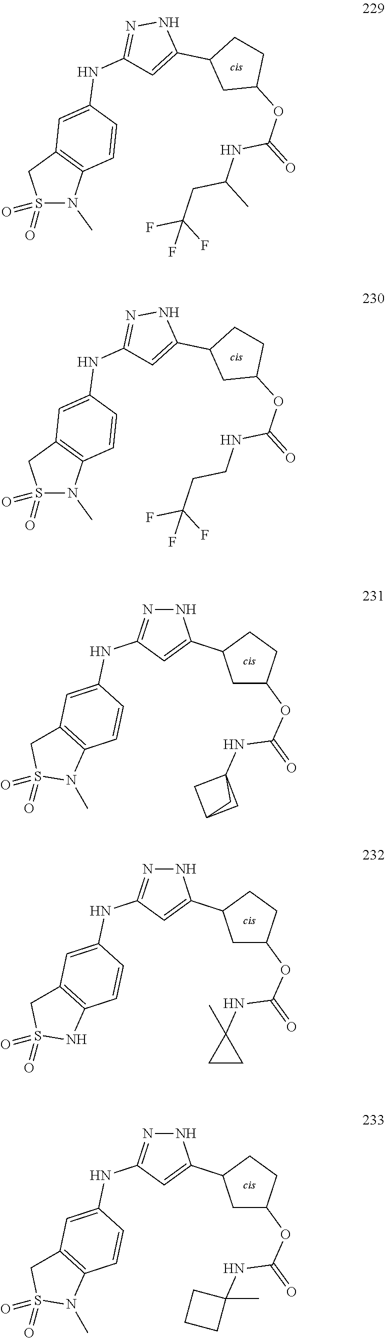 [SMILES: CC(CC(F)(F)F)NC(=O)OC1CCC(c2cc(Nc3ccc4c(c3)CS(=O)(=O)N4C)n[nH]2)C1 ; CN1c2ccc(Nc3cc(C4CCC(OC(=O)NCCC(F)(F)F)C4)[nH]n3)cc2CS1(=O)=O ; CN1c2ccc(Nc3cc(C4CCC(OC(=O)NC56CC(C5)C6)C4)[nH]n3)cc2CS1(=O)=O ; CC1(NC(=O)OC2CCC(c3cc(Nc4ccc5c(c4)CS(=O)(=O)N5)n[nH]3)C2)CC1 ; CN1c2ccc(Nc3cc(C4CCC(OC(=O)NC5(C)CCC5)C4)[nH]n3)cc2CS1(=O)=O] 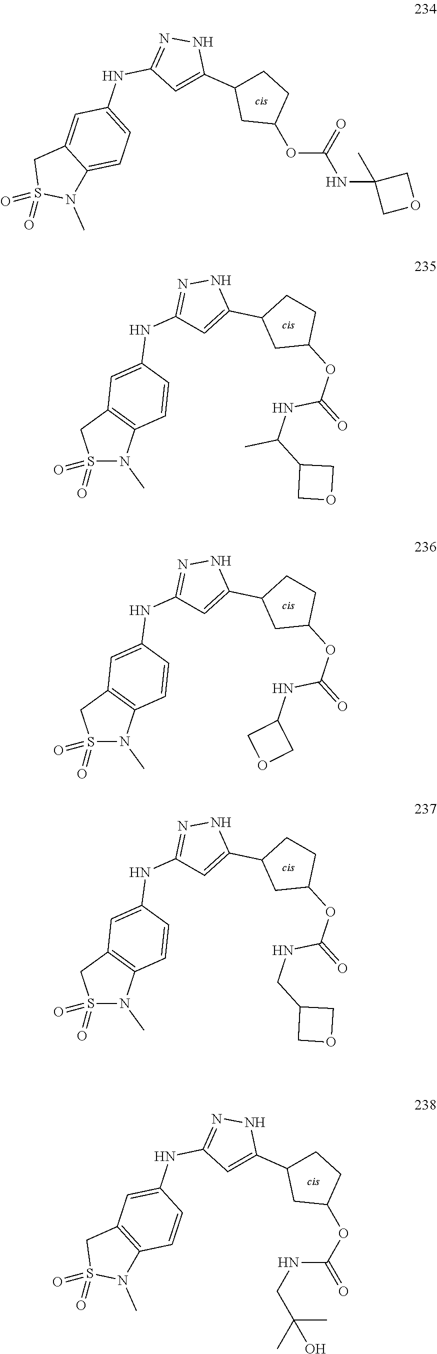 [SMILES: CN1c2ccc(Nc3cc(C4CCC(OC(=O)NC5(C)COC5)C4)[nH]n3)cc2CS1(=O)=O ; CC(NC(=O)OC1CCC(c2cc(Nc3ccc4c(c3)CS(=O)(=O)N4C)n[nH]2)C1)C1COC1 ; CN1c2ccc(Nc3cc(C4CCC(OC(=O)NC5COC5)C4)[nH]n3)cc2CS1(=O)=O ; CN1c2ccc(Nc3cc(C4CCC(OC(=O)NCC5COC5)C4)[nH]n3)cc2CS1(=O)=O ; CN1c2ccc(Nc3cc(C4CCC(OC(=O)NCC(C)(C)O)C4)[nH]n3)cc2CS1(=O)=O] 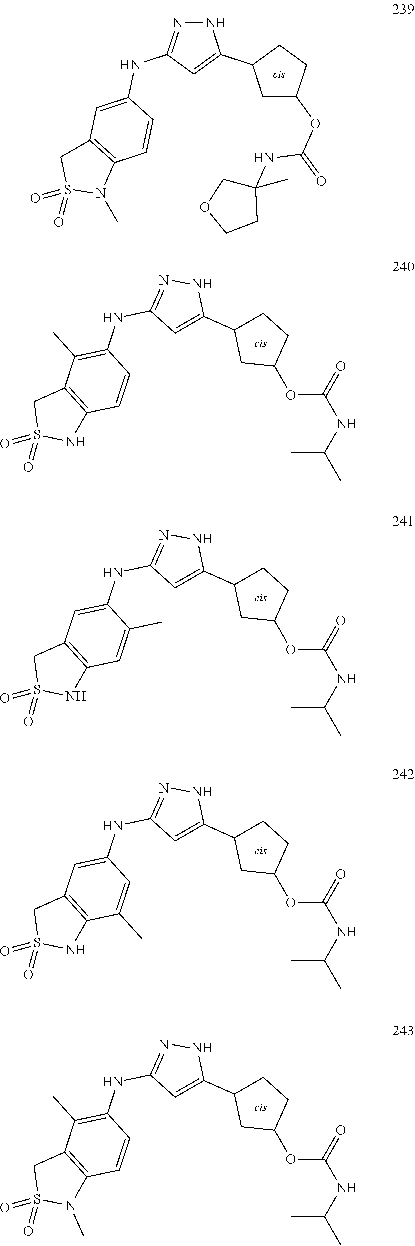 [SMILES: CN1c2ccc(Nc3cc(C4CCC(OC(=O)NC5(C)CCOC5)C4)[nH]n3)cc2CS1(=O)=O ; Cc1c(Nc2cc(C3CCC(OC(=O)NC(C)C)C3)[nH]n2)ccc2c1CS(=O)(=O)N2 ; Cc1cc2c(cc1Nc1cc(C3CCC(OC(=O)NC(C)C)C3)[nH]n1)CS(=O)(=O)N2 ; Cc1cc(Nc2cc(C3CCC(OC(=O)NC(C)C)C3)[nH]n2)cc2c1NS(=O)(=O)C2 ; Cc1c(Nc2cc(C3CCC(OC(=O)NC(C)C)C3)[nH]n2)ccc2c1CS(=O)(=O)N2C] 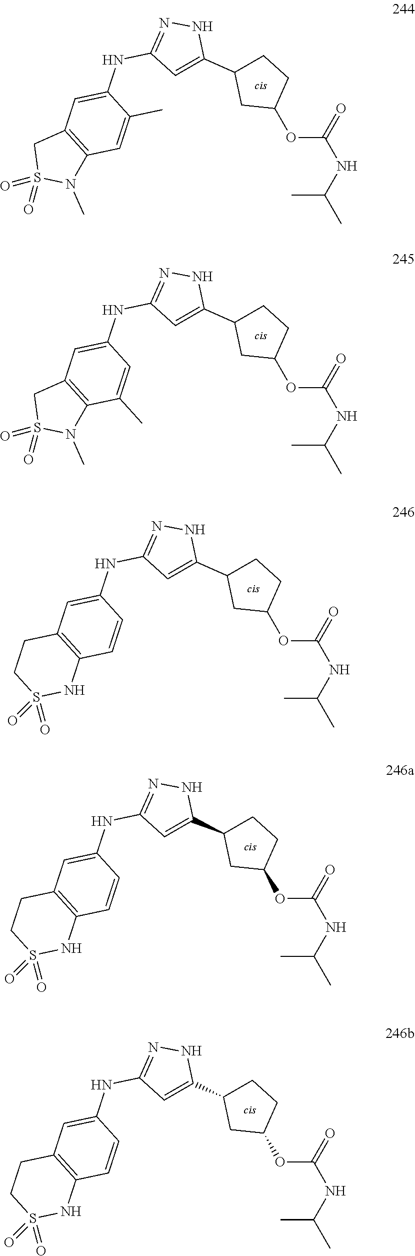 [SMILES: Cc1cc2c(cc1Nc1cc(C3CCC(OC(=O)NC(C)C)C3)[nH]n1)CS(=O)(=O)N2C ; Cc1cc(Nc2cc(C3CCC(OC(=O)NC(C)C)C3)[nH]n2)cc2c1N(C)S(=O)(=O)C2 ; CC(C)NC(=O)OC1CCC(c2cc(Nc3ccc4c(c3)CCS(=O)(=O)N4)n[nH]2)C1 ; CC(C)NC(=O)O[C@@H]1CC[C@H](c2cc(Nc3ccc4c(c3)CCS(=O)(=O)N4)n[nH]2)C1 ; CC(C)NC(=O)O[C@H]1CC[C@@H](c2cc(Nc3ccc4c(c3)CCS(=O)(=O)N4)n[nH]2)C1] 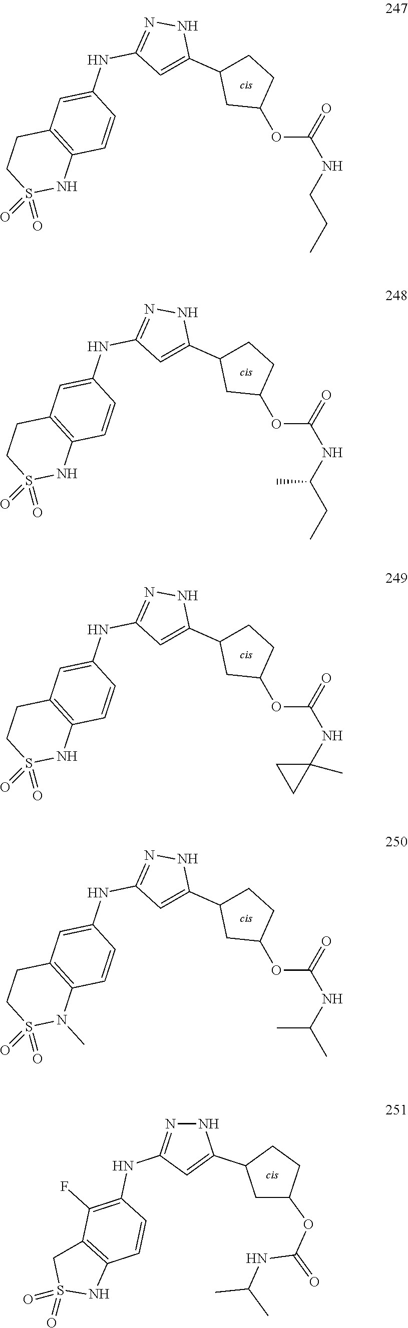 [SMILES: CCCNC(=O)OC1CCC(c2cc(Nc3ccc4c(c3)CCS(=O)(=O)N4)n[nH]2)C1 ; CC[C@H](C)NC(=O)OC1CCC(c2cc(Nc3ccc4c(c3)CCS(=O)(=O)N4)n[nH]2)C1 ; CC1(NC(=O)OC2CCC(c3cc(Nc4ccc5c(c4)CCS(=O)(=O)N5)n[nH]3)C2)CC1 ; CC(C)NC(=O)OC1CCC(c2cc(Nc3ccc4c(c3)CCS(=O)(=O)N4C)n[nH]2)C1 ; CC(C)NC(=O)OC1CCC(c2cc(Nc3ccc4c(c3F)CS(=O)(=O)N4)n[nH]2)C1] 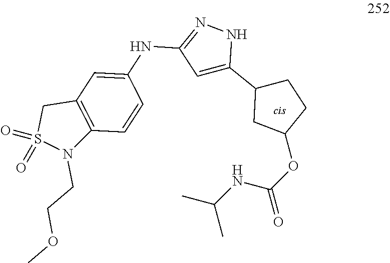 [SMILES: COCCN1c2ccc(Nc3cc(C4CCC(OC(=O)NC(C)C)C4)[nH]n3)cc2CS1(=O)=O] 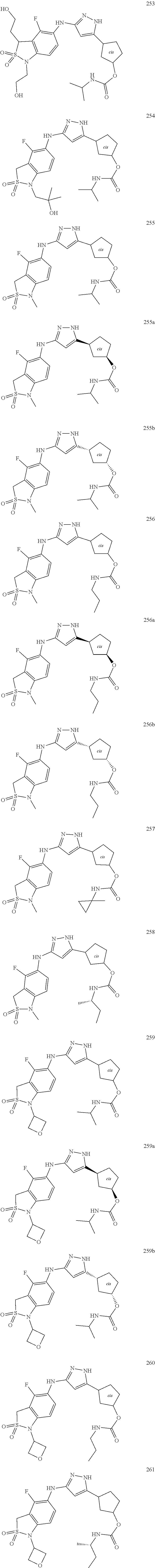 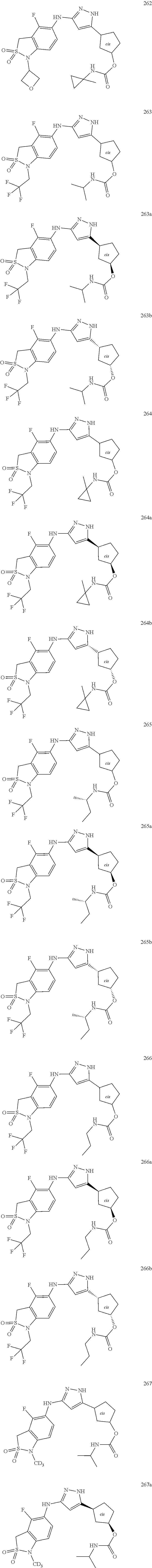 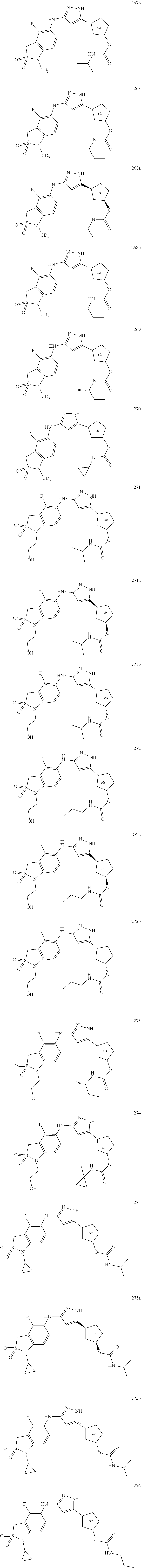 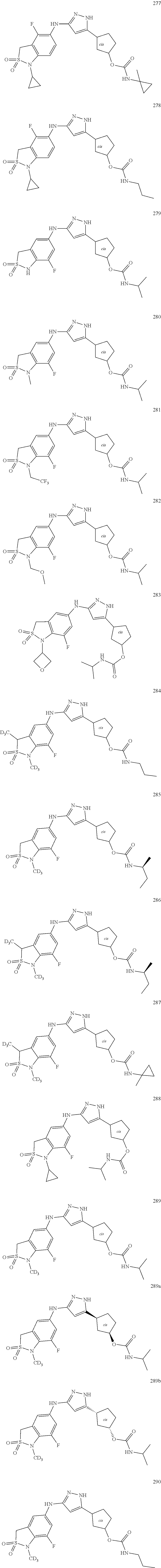 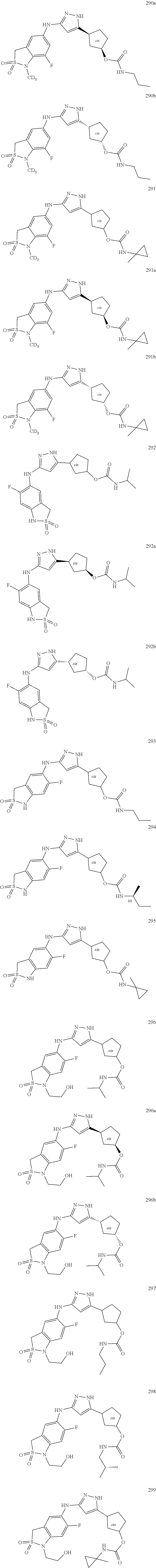 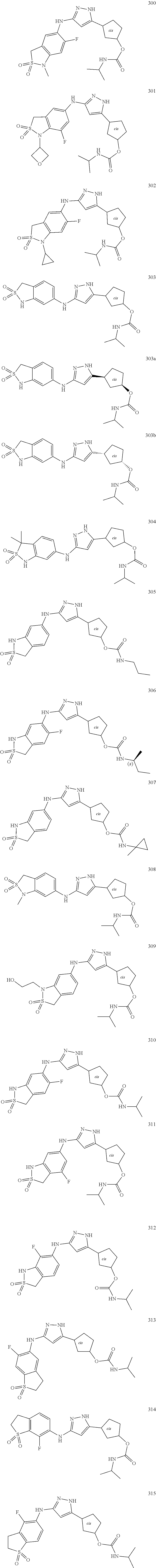 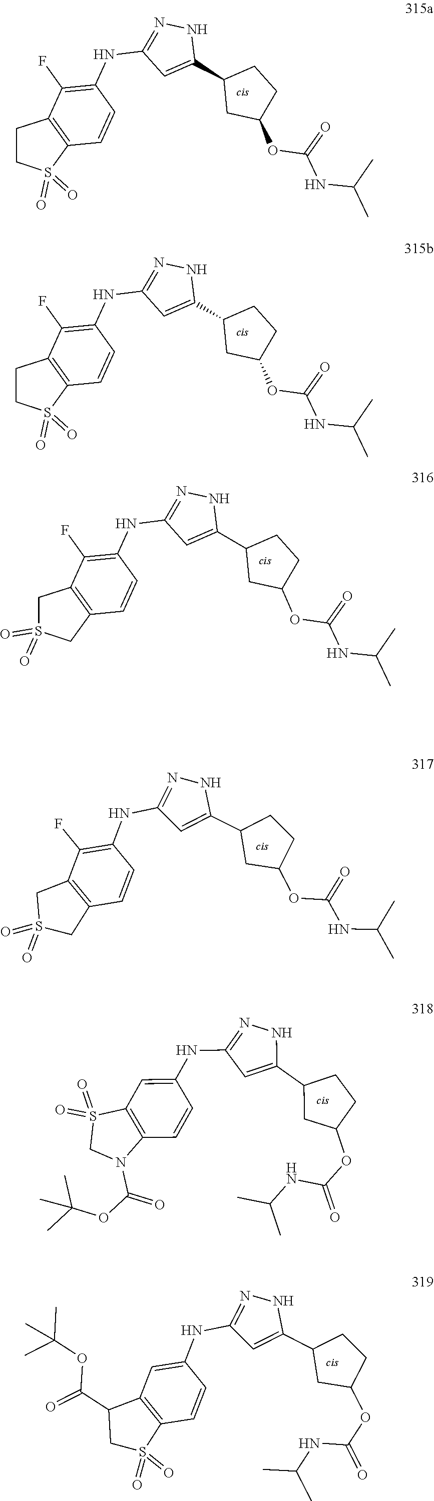 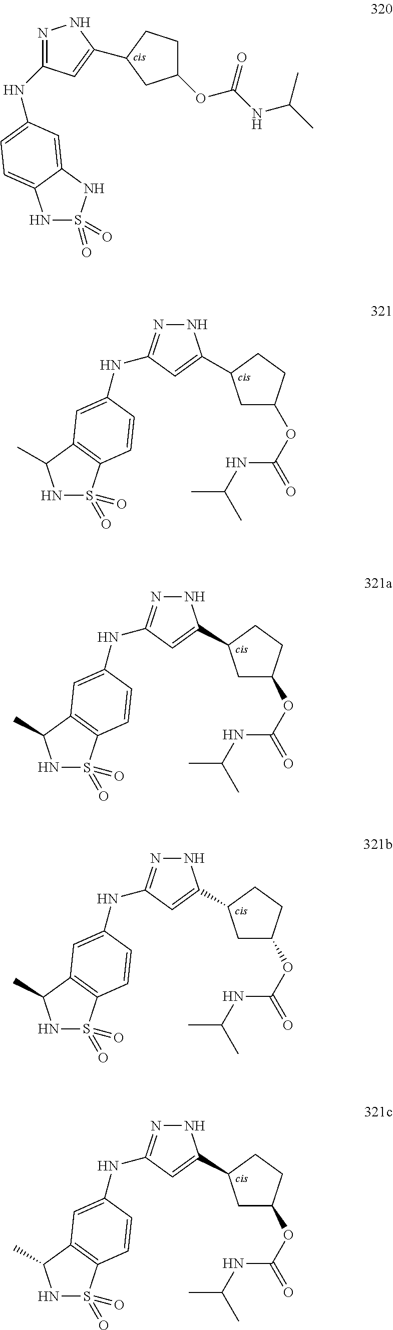 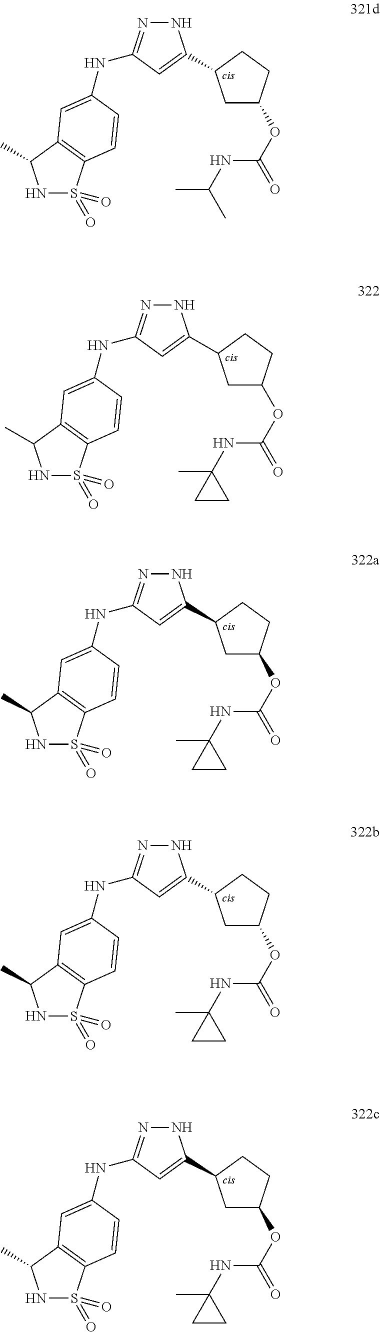 [SMILES: CC(C)NC(=O)O[C@H]1CC[C@@H](c2cc(Nc3ccc4c(c3)[C@@H](C)NS4(=O)=O)n[nH]2)C1 ; CC1NS(=O)(=O)c2ccc(Nc3cc(C4CCC(OC(=O)NC5(C)CC5)C4)[nH]n3)cc21 ; C[C@@H]1NS(=O)(=O)c2ccc(Nc3cc([C@H]4CC[C@@H](OC(=O)NC5(C)CC5)C4)[nH]n3)cc21 ; C[C@@H]1NS(=O)(=O)c2ccc(Nc3cc([C@@H]4CC[C@H](OC(=O)NC5(C)CC5)C4)[nH]n3)cc21 ; C[C@H]1NS(=O)(=O)c2ccc(Nc3cc([C@H]4CC[C@@H](OC(=O)NC5(C)CC5)C4)[nH]n3)cc21] 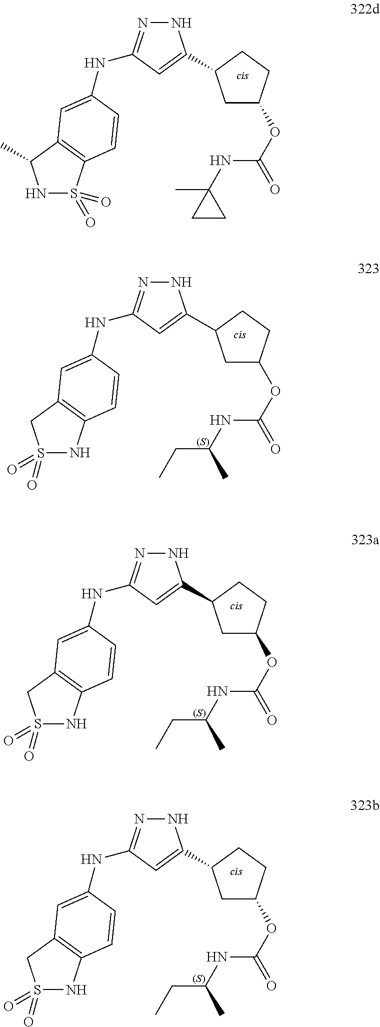 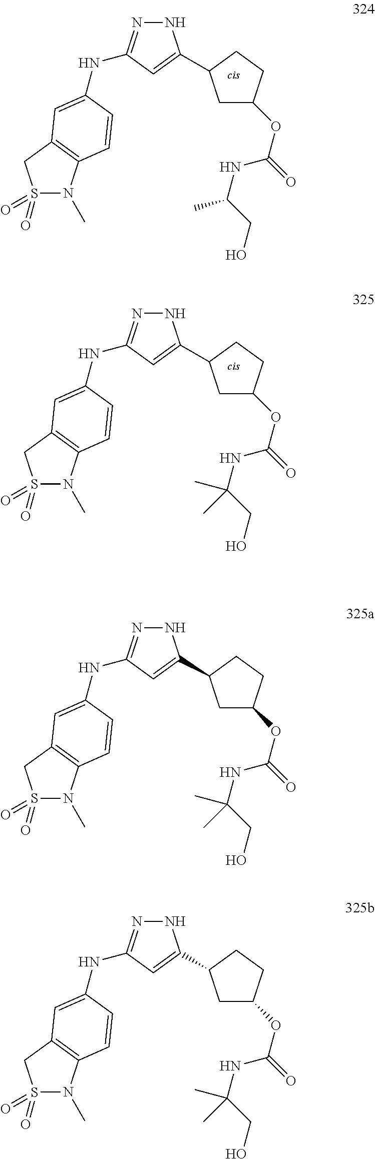 [SMILES: C[C@@H](CO)NC(=O)OC1CCC(c2cc(Nc3ccc4c(c3)CS(=O)(=O)N4C)n[nH]2)C1 ; CN1c2ccc(Nc3cc(C4CCC(OC(=O)NC(C)(C)CO)C4)[nH]n3)cc2CS1(=O)=O ; CN1c2ccc(Nc3cc([C@H]4CC[C@@H](OC(=O)NC(C)(C)CO)C4)[nH]n3)cc2CS1(=O)=O ; CN1c2ccc(Nc3cc([C@@H]4CC[C@H](OC(=O)NC(C)(C)CO)C4)[nH]n3)cc2CS1(=O)=O] 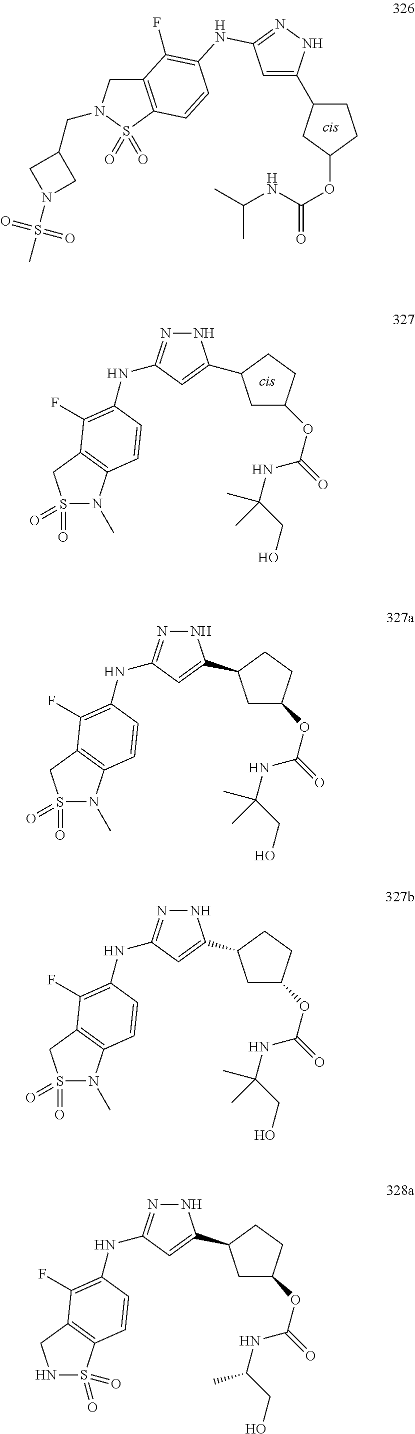 [SMILES: CC(C)NC(=O)OC1CCC(c2cc(Nc3ccc4c(c3F)CN(CC3CN(S(C)(=O)=O)C3)S4(=O)=O)n[nH]2)C1 ; CN1c2ccc(Nc3cc(C4CCC(OC(=O)NC(C)(C)CO)C4)[nH]n3)c(F)c2CS1(=O)=O ; CN1c2ccc(Nc3cc([C@H]4CC[C@@H](OC(=O)NC(C)(C)CO)C4)[nH]n3)c(F)c2CS1(=O)=O ; CN1c2ccc(Nc3cc([C@@H]4CC[C@H](OC(=O)NC(C)(C)CO)C4)[nH]n3)c(F)c2CS1(=O)=O ; C[C@@H](CO)NC(=O)O[C@@H]1CC[C@H](c2cc(Nc3ccc4c(c3F)CNS4(=O)=O)n[nH]2)C1] 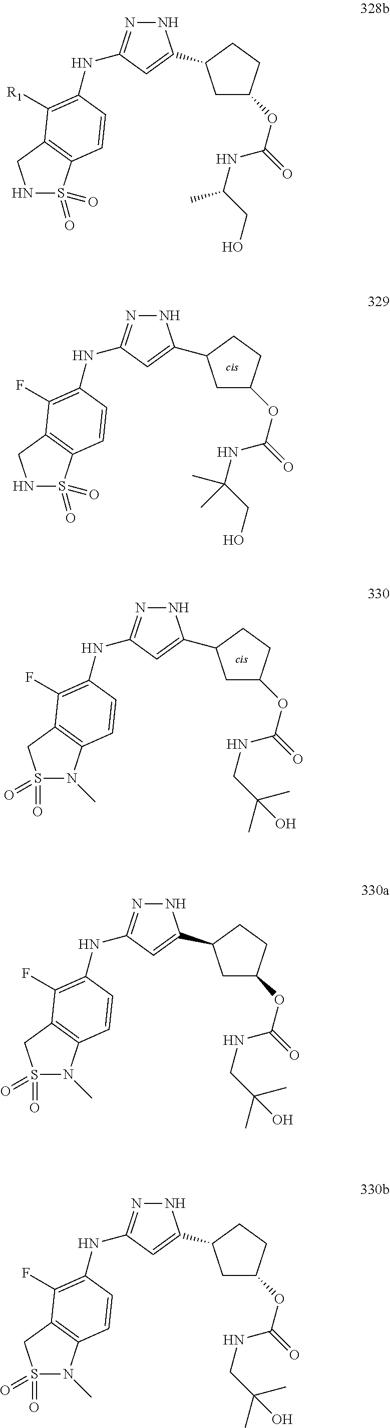 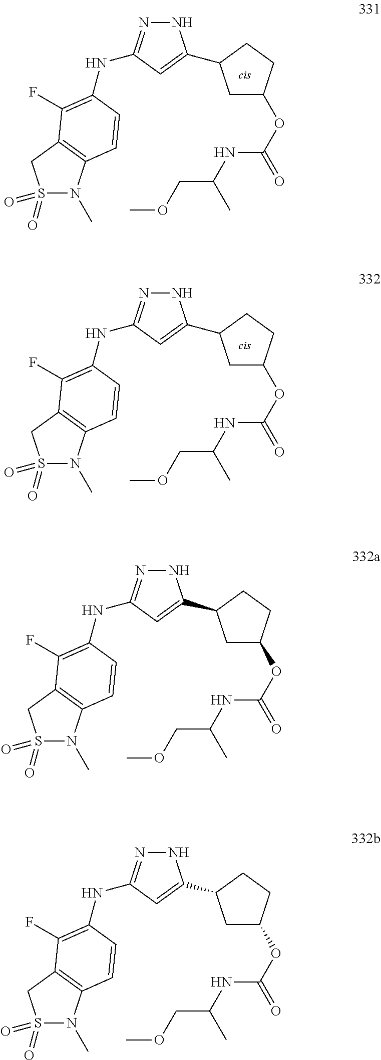 [SMILES: COCC(C)NC(=O)OC1CCC(c2cc(Nc3ccc4c(c3F)CS(=O)(=O)N4C)n[nH]2)C1 ; COCC(C)NC(=O)OC1CCC(c2cc(Nc3ccc4c(c3F)CS(=O)(=O)N4C)n[nH]2)C1 ; COCC(C)NC(=O)O[C@@H]1CC[C@H](c2cc(Nc3ccc4c(c3F)CS(=O)(=O)N4C)n[nH]2)C1 ; COCC(C)NC(=O)O[C@H]1CC[C@@H](c2cc(Nc3ccc4c(c3F)CS(=O)(=O)N4C)n[nH]2)C1] 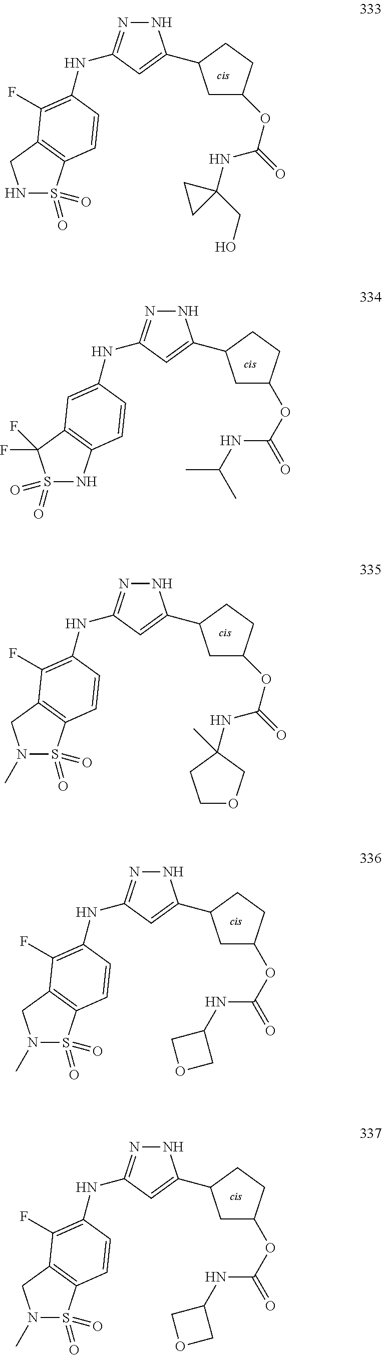 [SMILES: O=C(NC1(CO)CC1)OC1CCC(c2cc(Nc3ccc4c(c3F)CNS4(=O)=O)n[nH]2)C1 ; CC(C)NC(=O)OC1CCC(c2cc(Nc3ccc4c(c3)C(F)(F)S(=O)(=O)N4)n[nH]2)C1 ; CN1Cc2c(ccc(Nc3cc(C4CCC(OC(=O)NC5(C)CCOC5)C4)[nH]n3)c2F)S1(=O)=O ; CN1Cc2c(ccc(Nc3cc(C4CCC(OC(=O)NC5COC5)C4)[nH]n3)c2F)S1(=O)=O ; CN1Cc2c(ccc(Nc3cc(C4CCC(OC(=O)NC5COC5)C4)[nH]n3)c2F)S1(=O)=O] 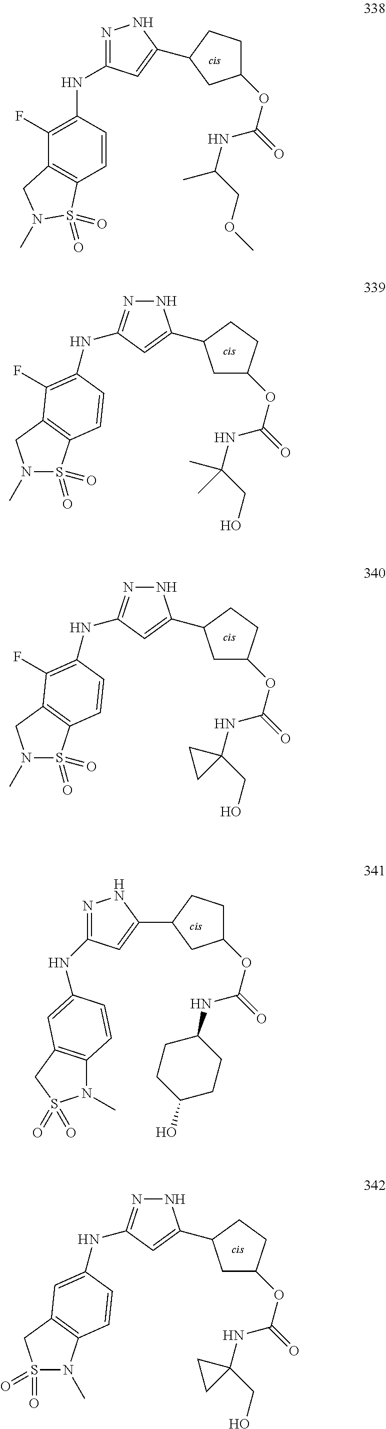 [SMILES: COCC(C)NC(=O)OC1CCC(c2cc(Nc3ccc4c(c3F)CN(C)S4(=O)=O)n[nH]2)C1 ; CN1Cc2c(ccc(Nc3cc(C4CCC(OC(=O)NC(C)(C)CO)C4)[nH]n3)c2F)S1(=O)=O ; CN1Cc2c(ccc(Nc3cc(C4CCC(OC(=O)NC5(CO)CC5)C4)[nH]n3)c2F)S1(=O)=O ; CN1c2ccc(Nc3cc(C4CCC(OC(=O)N[C@H]5CC[C@H](O)CC5)C4)[nH]n3)cc2CS1(=O)=O ; CN1c2ccc(Nc3cc(C4CCC(OC(=O)NC5(CO)CC5)C4)[nH]n3)cc2CS1(=O)=O] 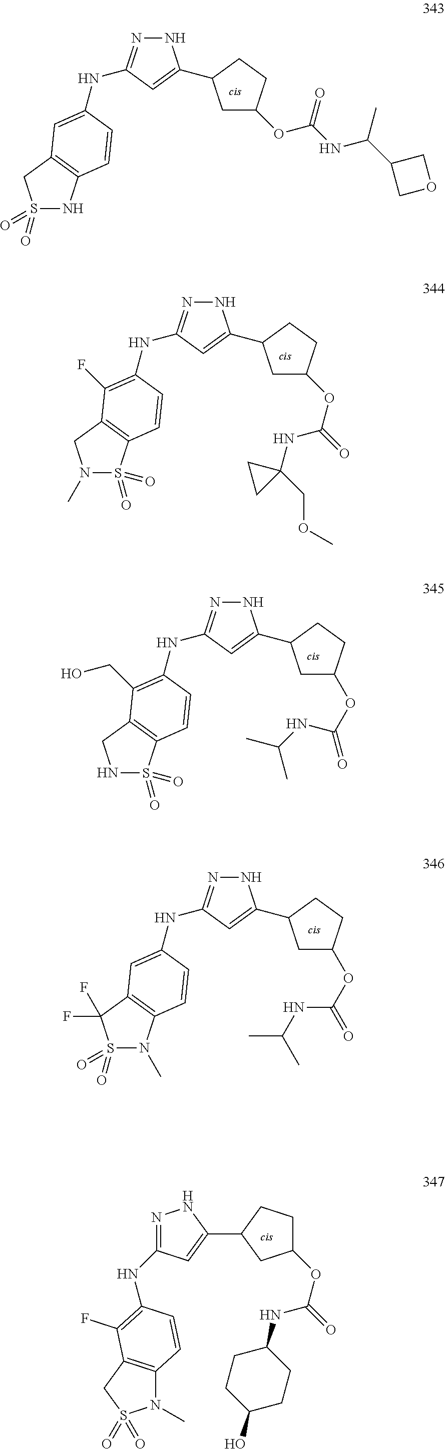 [SMILES: CC(NC(=O)OC1CCC(c2cc(Nc3ccc4c(c3)CS(=O)(=O)N4)n[nH]2)C1)C1COC1 ; COCC1(NC(=O)OC2CCC(c3cc(Nc4ccc5c(c4F)CN(C)S5(=O)=O)n[nH]3)C2)CC1 ; CC(C)NC(=O)OC1CCC(c2cc(Nc3ccc4c(c3CO)CNS4(=O)=O)n[nH]2)C1 ; CC(C)NC(=O)OC1CCC(c2cc(Nc3ccc4c(c3)C(F)(F)S(=O)(=O)N4C)n[nH]2)C1 ; CN1c2ccc(Nc3cc(C4CCC(OC(=O)N[C@H]5CC[C@@H](O)CC5)C4)[nH]n3)c(F)c2CS1(=O)=O] 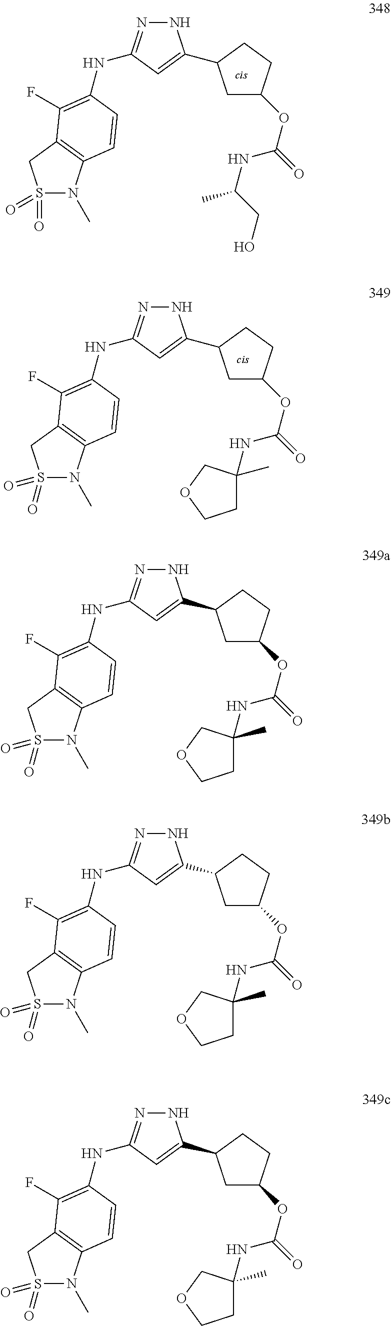 [SMILES: C[C@@H](CO)NC(=O)OC1CCC(c2cc(Nc3ccc4c(c3F)CS(=O)(=O)N4C)n[nH]2)C1 ; CN1c2ccc(Nc3cc(C4CCC(OC(=O)NC5(C)CCOC5)C4)[nH]n3)c(F)c2CS1(=O)=O ; CN1c2ccc(Nc3cc([C@H]4CC[C@@H](OC(=O)N[C@]5(C)CCOC5)C4)[nH]n3)c(F)c2CS1(=O)=O ; CN1c2ccc(Nc3cc([C@@H]4CC[C@H](OC(=O)N[C@]5(C)CCOC5)C4)[nH]n3)c(F)c2CS1(=O)=O ; CN1c2ccc(Nc3cc([C@H]4CC[C@@H](OC(=O)N[C@@]5(C)CCOC5)C4)[nH]n3)c(F)c2CS1(=O)=O] 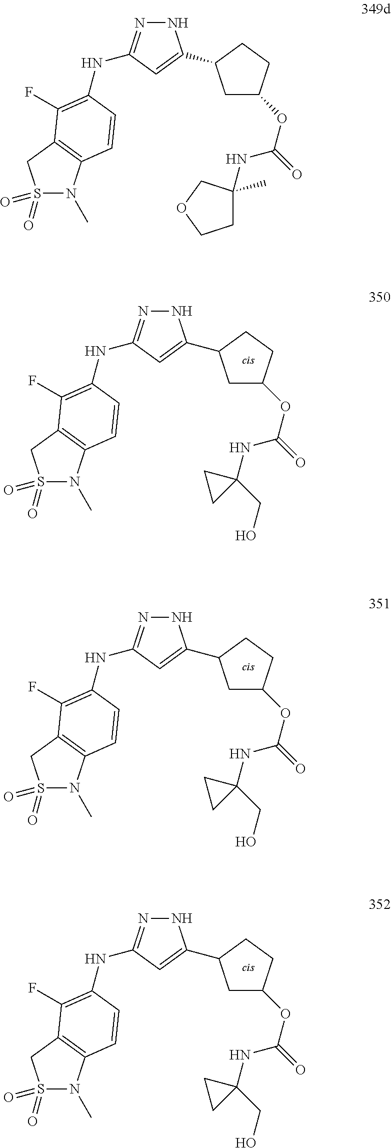 [SMILES: CN1c2ccc(Nc3cc([C@@H]4CC[C@H](OC(=O)N[C@@]5(C)CCOC5)C4)[nH]n3)c(F)c2CS1(=O)=O ; CN1c2ccc(Nc3cc(C4CCC(OC(=O)NC5(CO)CC5)C4)[nH]n3)c(F)c2CS1(=O)=O ; CN1c2ccc(Nc3cc(C4CCC(OC(=O)NC5(CO)CC5)C4)[nH]n3)c(F)c2CS1(=O)=O ; CN1c2ccc(Nc3cc(C4CCC(OC(=O)NC5(CO)CC5)C4)[nH]n3)c(F)c2CS1(=O)=O] 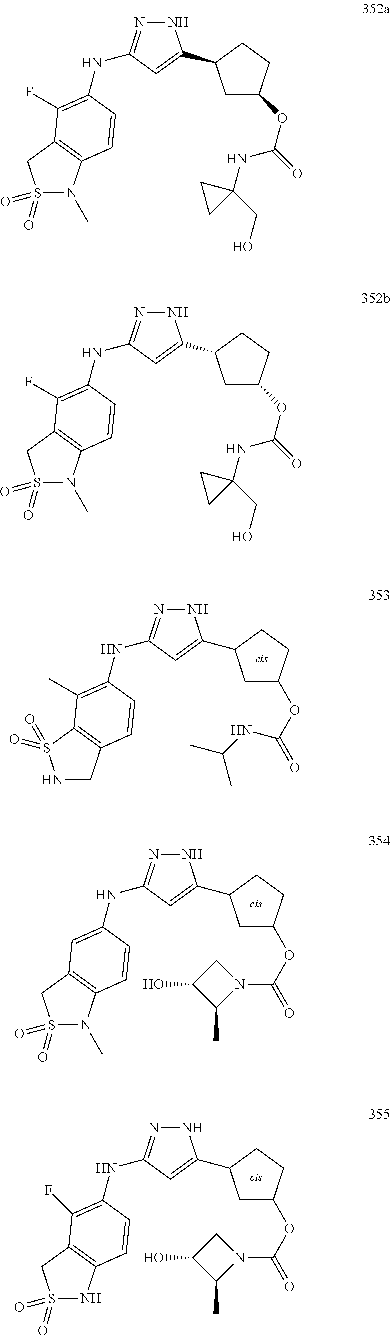 [SMILES: CN1c2ccc(Nc3cc([C@H]4CC[C@@H](OC(=O)NC5(CO)CC5)C4)[nH]n3)c(F)c2CS1(=O)=O ; CN1c2ccc(Nc3cc([C@@H]4CC[C@H](OC(=O)NC5(CO)CC5)C4)[nH]n3)c(F)c2CS1(=O)=O ; Cc1c(Nc2cc(C3CCC(OC(=O)NC(C)C)C3)[nH]n2)ccc2c1S(=O)(=O)NC2 ; C[C@H]1[C@H](O)CN1C(=O)OC1CCC(c2cc(Nc3ccc4c(c3)CS(=O)(=O)N4C)n[nH]2)C1 ; C[C@H]1[C@H](O)CN1C(=O)OC1CCC(c2cc(Nc3ccc4c(c3F)CS(=O)(=O)N4)n[nH]2)C1] 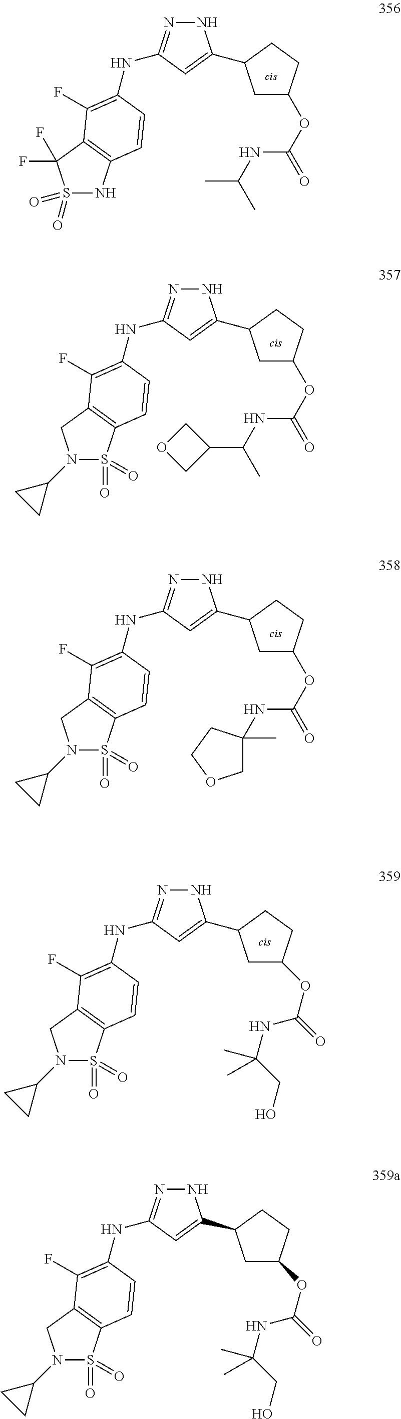 [SMILES: CC(C)NC(=O)OC1CCC(c2cc(Nc3ccc4c(c3F)C(F)(F)S(=O)(=O)N4)n[nH]2)C1 ; CC(NC(=O)OC1CCC(c2cc(Nc3ccc4c(c3F)CN(C3CC3)S4(=O)=O)n[nH]2)C1)C1COC1 ; CC1(NC(=O)OC2CCC(c3cc(Nc4ccc5c(c4F)CN(C4CC4)S5(=O)=O)n[nH]3)C2)CCOC1 ; CC(C)(CO)NC(=O)OC1CCC(c2cc(Nc3ccc4c(c3F)CN(C3CC3)S4(=O)=O)n[nH]2)C1 ; CC(C)(CO)NC(=O)O[C@@H]1CC[C@H](c2cc(Nc3ccc4c(c3F)CN(C3CC3)S4(=O)=O)n[nH]2)C1] 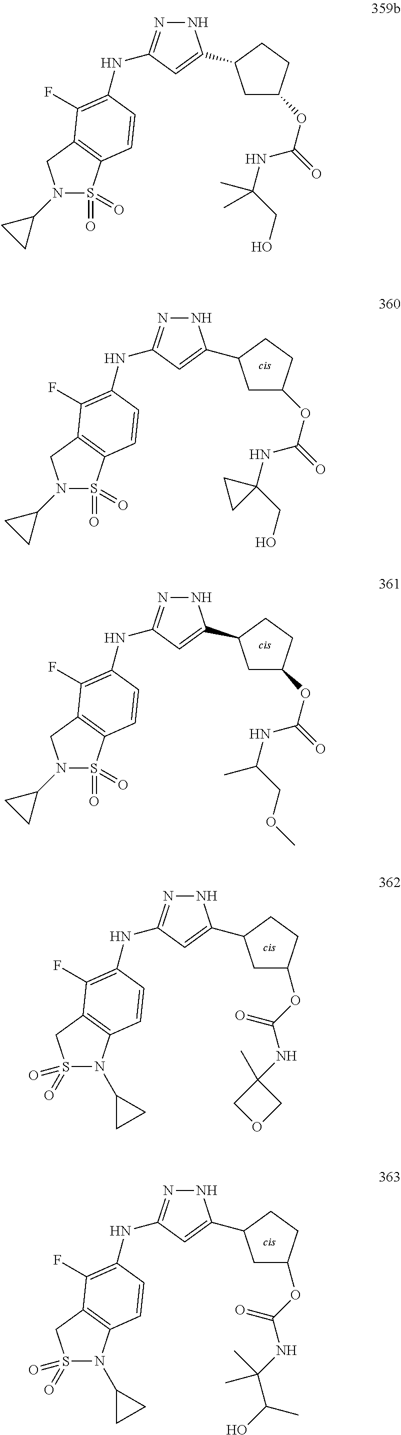 [SMILES: CC(C)(CO)NC(=O)O[C@H]1CC[C@@H](c2cc(Nc3ccc4c(c3F)CN(C3CC3)S4(=O)=O)n[nH]2)C1 ; O=C(NC1(CO)CC1)OC1CCC(c2cc(Nc3ccc4c(c3F)CN(C3CC3)S4(=O)=O)n[nH]2)C1 ; COCC(C)NC(=O)O[C@@H]1CC[C@H](c2cc(Nc3ccc4c(c3F)CN(C3CC3)S4(=O)=O)n[nH]2)C1 ; CC1(NC(=O)OC2CCC(c3cc(Nc4ccc5c(c4F)CS(=O)(=O)N5C4CC4)n[nH]3)C2)COC1 ; CC(O)C(C)(C)NC(=O)OC1CCC(c2cc(Nc3ccc4c(c3F)CS(=O)(=O)N4C3CC3)n[nH]2)C1] 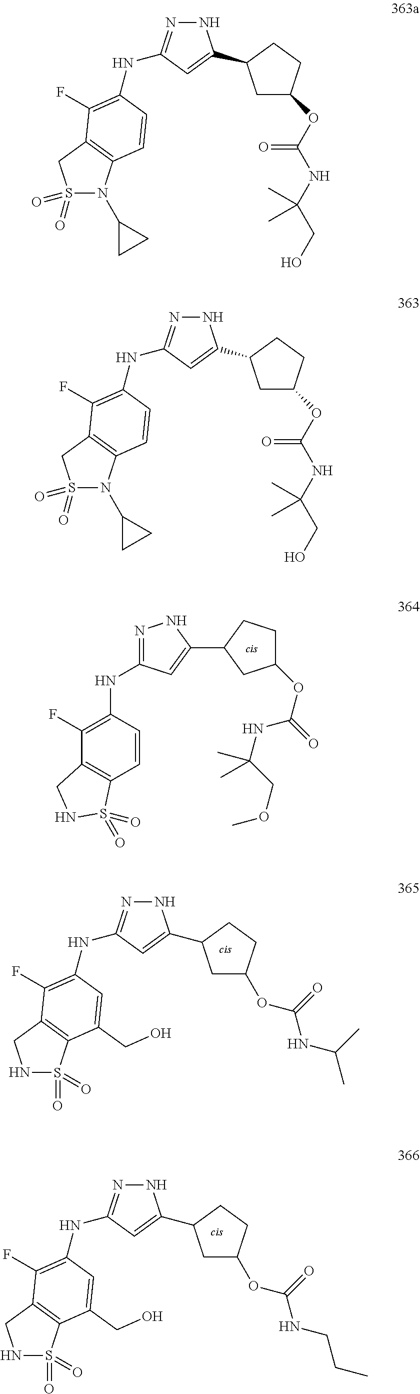 [SMILES: CC(C)(CO)NC(=O)O[C@@H]1CC[C@H](c2cc(Nc3ccc4c(c3F)CS(=O)(=O)N4C3CC3)n[nH]2)C1 ; CC(C)(CO)NC(=O)O[C@H]1CC[C@@H](c2cc(Nc3ccc4c(c3F)CS(=O)(=O)N4C3CC3)n[nH]2)C1 ; COCC(C)(C)NC(=O)OC1CCC(c2cc(Nc3ccc4c(c3F)CNS4(=O)=O)n[nH]2)C1 ; CC(C)NC(=O)OC1CCC(c2cc(Nc3cc(CO)c4c(c3F)CNS4(=O)=O)n[nH]2)C1 ; CCCNC(=O)OC1CCC(c2cc(Nc3cc(CO)c4c(c3F)CNS4(=O)=O)n[nH]2)C1] 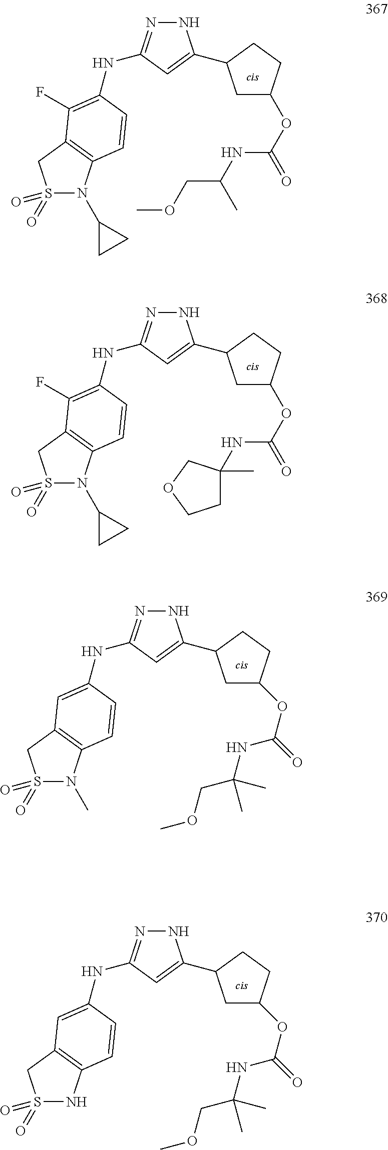 [SMILES: COCC(C)NC(=O)OC1CCC(c2cc(Nc3ccc4c(c3F)CS(=O)(=O)N4C3CC3)n[nH]2)C1 ; CC1(NC(=O)OC2CCC(c3cc(Nc4ccc5c(c4F)CS(=O)(=O)N5C4CC4)n[nH]3)C2)CCOC1 ; COCC(C)(C)NC(=O)OC1CCC(c2cc(Nc3ccc4c(c3)CS(=O)(=O)N4C)n[nH]2)C1 ; COCC(C)(C)NC(=O)OC1CCC(c2cc(Nc3ccc4c(c3)CS(=O)(=O)N4)n[nH]2)C1] 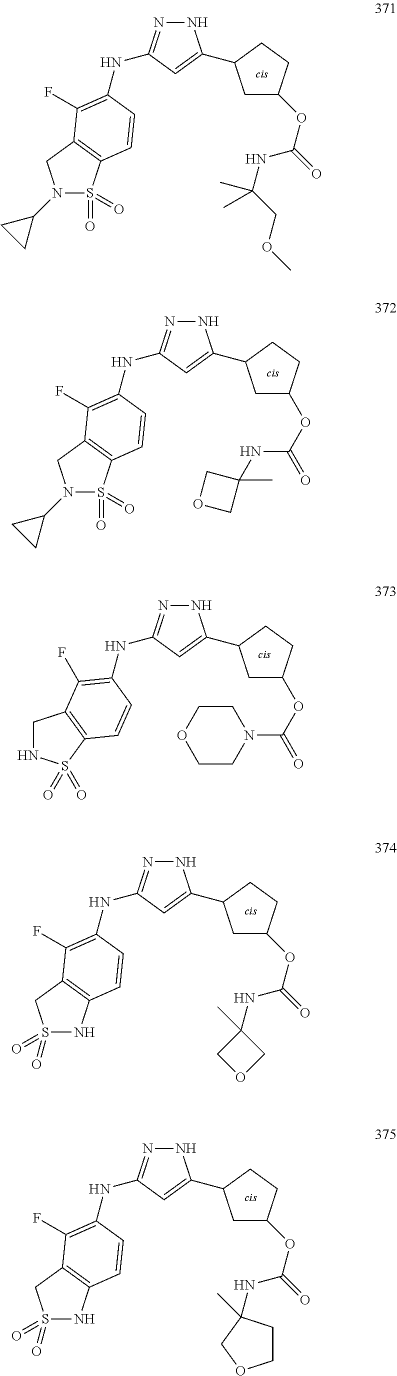 [SMILES: COCC(C)(C)NC(=O)OC1CCC(c2cc(Nc3ccc4c(c3F)CN(C3CC3)S4(=O)=O)n[nH]2)C1 ; CC1(NC(=O)OC2CCC(c3cc(Nc4ccc5c(c4F)CN(C4CC4)S5(=O)=O)n[nH]3)C2)COC1 ; O=C(OC1CCC(c2cc(Nc3ccc4c(c3F)CNS4(=O)=O)n[nH]2)C1)N1CCOCC1 ; CC1(NC(=O)OC2CCC(c3cc(Nc4ccc5c(c4F)CS(=O)(=O)N5)n[nH]3)C2)COC1 ; CC1(NC(=O)OC2CCC(c3cc(Nc4ccc5c(c4F)CS(=O)(=O)N5)n[nH]3)C2)CCOC1] 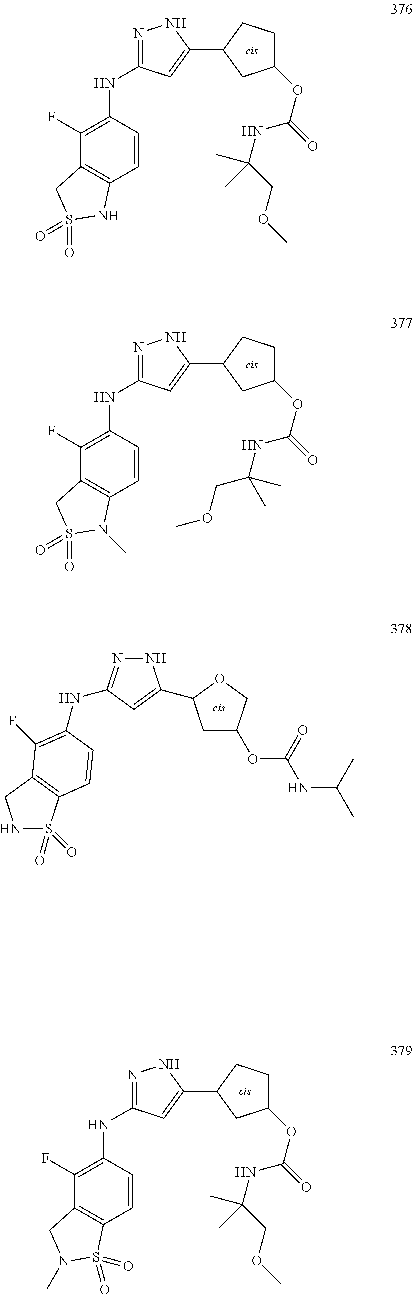 [SMILES: COCC(C)(C)NC(=O)OC1CCC(c2cc(Nc3ccc4c(c3F)CS(=O)(=O)N4)n[nH]2)C1 ; COCC(C)(C)NC(=O)OC1CCC(c2cc(Nc3ccc4c(c3F)CS(=O)(=O)N4C)n[nH]2)C1 ; CC(C)NC(=O)OC1COC(c2cc(Nc3ccc4c(c3F)CNS4(=O)=O)n[nH]2)C1 ; COCC(C)(C)NC(=O)OC1CCC(c2cc(Nc3ccc4c(c3F)CN(C)S4(=O)=O)n[nH]2)C1] 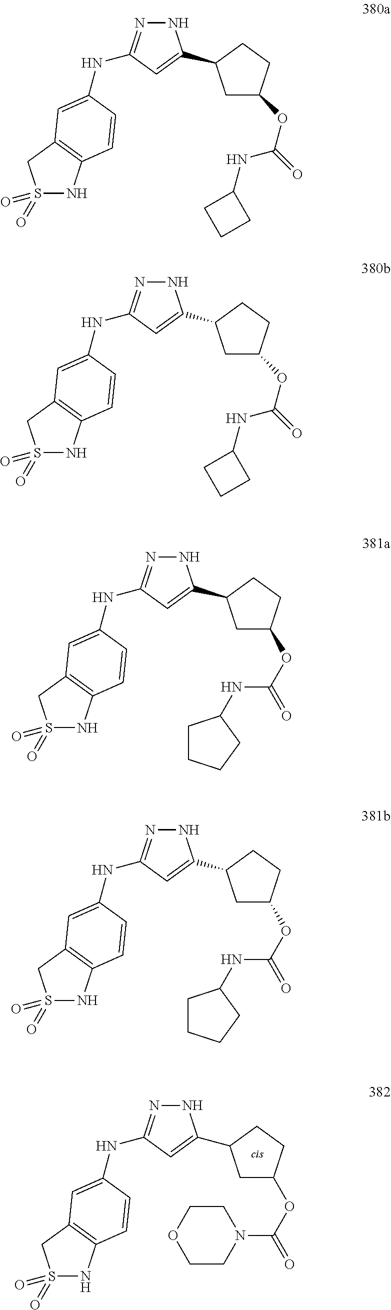 [SMILES: O=C(NC1CCC1)O[C@@H]1CC[C@H](c2cc(Nc3ccc4c(c3)CS(=O)(=O)N4)n[nH]2)C1 ; O=C(NC1CCC1)O[C@H]1CC[C@@H](c2cc(Nc3ccc4c(c3)CS(=O)(=O)N4)n[nH]2)C1 ; O=C(NC1CCCC1)O[C@@H]1CC[C@H](c2cc(Nc3ccc4c(c3)CS(=O)(=O)N4)n[nH]2)C1 ; O=C(NC1CCCC1)O[C@H]1CC[C@@H](c2cc(Nc3ccc4c(c3)CS(=O)(=O)N4)n[nH]2)C1 ; O=C(OC1CCC(c2cc(Nc3ccc4c(c3)CS(=O)(=O)N4)n[nH]2)C1)N1CCOCC1] 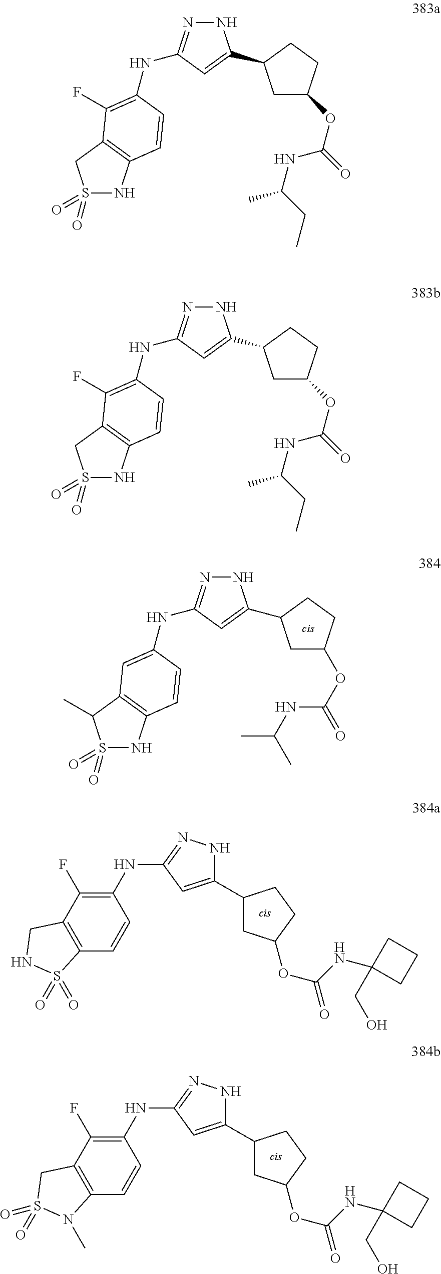 [SMILES: CC[C@H](C)NC(=O)O[C@@H]1CC[C@H](c2cc(Nc3ccc4c(c3F)CS(=O)(=O)N4)n[nH]2)C1 ; CC[C@H](C)NC(=O)O[C@H]1CC[C@@H](c2cc(Nc3ccc4c(c3F)CS(=O)(=O)N4)n[nH]2)C1 ; CC(C)NC(=O)OC1CCC(c2cc(Nc3ccc4c(c3)C(C)S(=O)(=O)N4)n[nH]2)C1 ; O=C(NC1(CO)CCC1)OC1CCC(c2cc(Nc3ccc4c(c3F)CNS4(=O)=O)n[nH]2)C1 ; CN1c2ccc(Nc3cc(C4CCC(OC(=O)NC5(CO)CCC5)C4)[nH]n3)c(F)c2CS1(=O)=O] 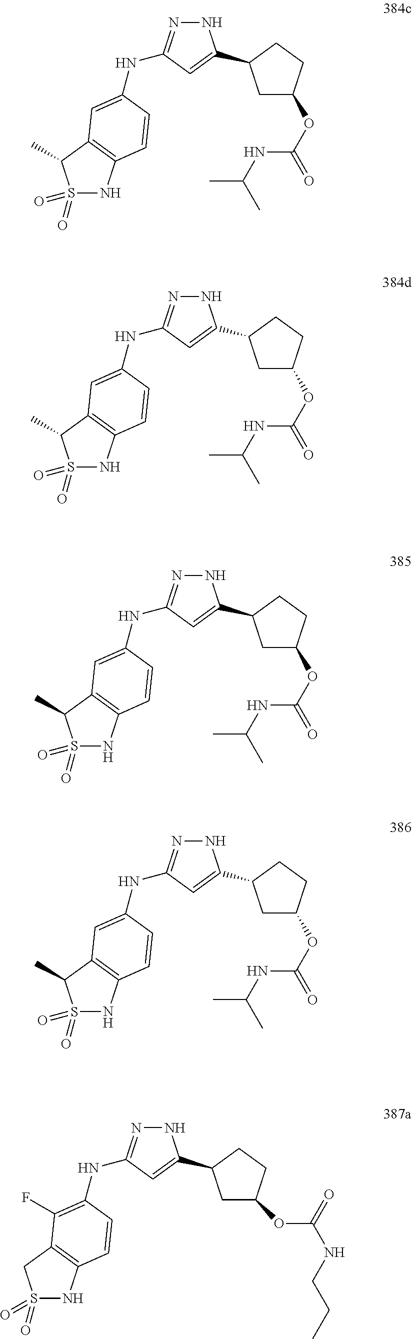 [SMILES: CC(C)NC(=O)O[C@@H]1CC[C@H](c2cc(Nc3ccc4c(c3)[C@@H](C)S(=O)(=O)N4)n[nH]2)C1 ; CC(C)NC(=O)O[C@H]1CC[C@@H](c2cc(Nc3ccc4c(c3)[C@@H](C)S(=O)(=O)N4)n[nH]2)C1 ; CC(C)NC(=O)O[C@@H]1CC[C@H](c2cc(Nc3ccc4c(c3)[C@H](C)S(=O)(=O)N4)n[nH]2)C1 ; CC(C)NC(=O)O[C@H]1CC[C@@H](c2cc(Nc3ccc4c(c3)[C@H](C)S(=O)(=O)N4)n[nH]2)C1 ; CCCNC(=O)O[C@@H]1CC[C@H](c2cc(Nc3ccc4c(c3F)CS(=O)(=O)N4)n[nH]2)C1] 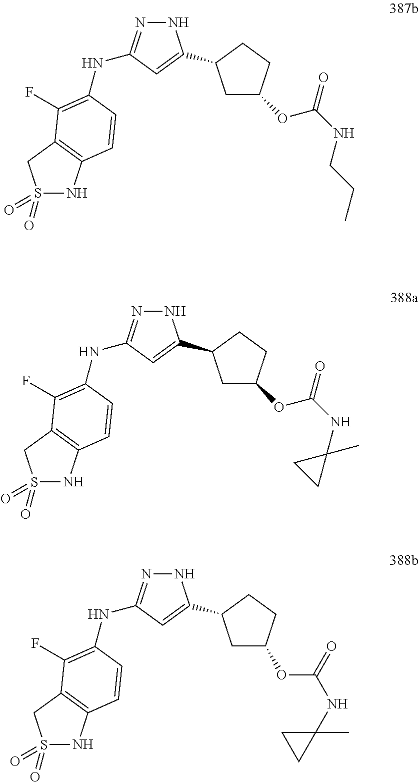 [SMILES: CCCNC(=O)O[C@H]1CC[C@@H](c2cc(Nc3ccc4c(c3F)CS(=O)(=O)N4)n[nH]2)C1 ; CC1(NC(=O)O[C@@H]2CC[C@H](c3cc(Nc4ccc5c(c4F)CS(=O)(=O)N5)n[nH]3)C2)CC1 ; CC1(NC(=O)O[C@H]2CC[C@@H](c3cc(Nc4ccc5c(c4F)CS(=O)(=O)N5)n[nH]3)C2)CC1] 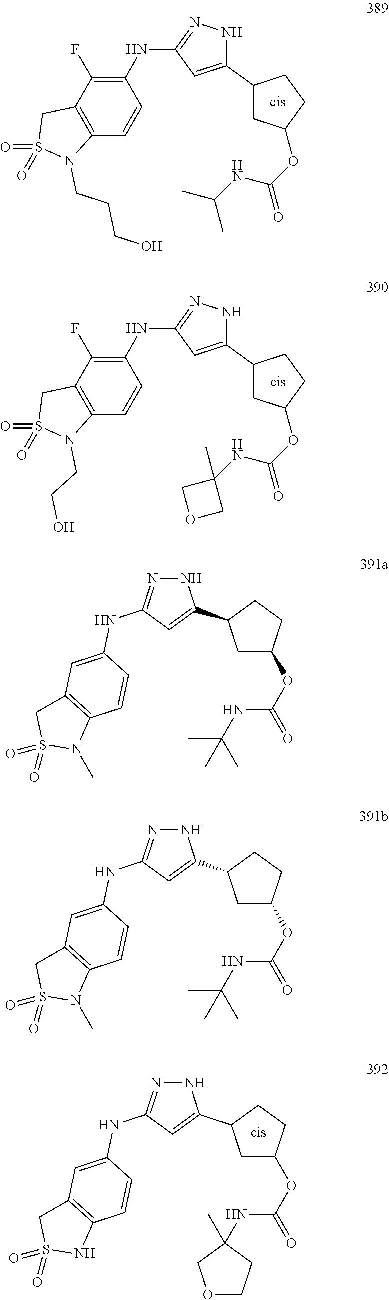 [SMILES: CC(C)NC(=O)OC1CCC(c2cc(Nc3ccc4c(c3F)CS(=O)(=O)N4CCCO)n[nH]2)C1 ; CC1(NC(=O)OC2CCC(c3cc(Nc4ccc5c(c4F)CS(=O)(=O)N5CCO)n[nH]3)C2)COC1 ; CN1c2ccc(Nc3cc([C@H]4CC[C@@H](OC(=O)NC(C)(C)C)C4)[nH]n3)cc2CS1(=O)=O ; CN1c2ccc(Nc3cc([C@@H]4CC[C@H](OC(=O)NC(C)(C)C)C4)[nH]n3)cc2CS1(=O)=O ; CC1(NC(=O)OC2CCC(c3cc(Nc4ccc5c(c4)CS(=O)(=O)N5)n[nH]3)C2)CCOC1] 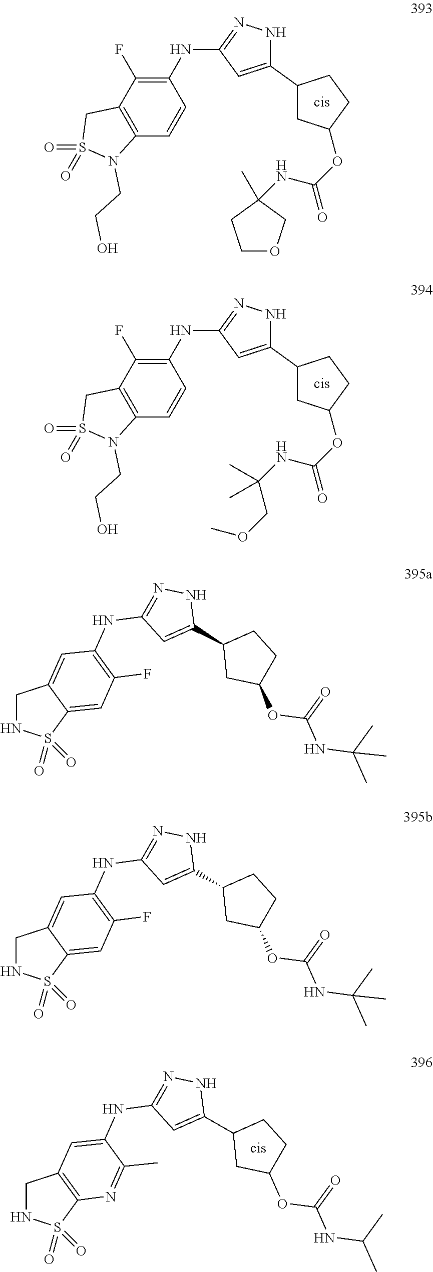 [SMILES: CC1(NC(=O)OC2CCC(c3cc(Nc4ccc5c(c4F)CS(=O)(=O)N5CCO)n[nH]3)C2)CCOC1 ; COCC(C)(C)NC(=O)OC1CCC(c2cc(Nc3ccc4c(c3F)CS(=O)(=O)N4CCO)n[nH]2)C1 ; CC(C)(C)NC(=O)O[C@@H]1CC[C@H](c2cc(Nc3cc4c(cc3F)S(=O)(=O)NC4)n[nH]2)C1 ; CC(C)(C)NC(=O)O[C@H]1CC[C@@H](c2cc(Nc3cc4c(cc3F)S(=O)(=O)NC4)n[nH]2)C1 ; Cc1nc2c(cc1Nc1cc(C3CCC(OC(=O)NC(C)C)C3)[nH]n1)CNS2(=O)=O] 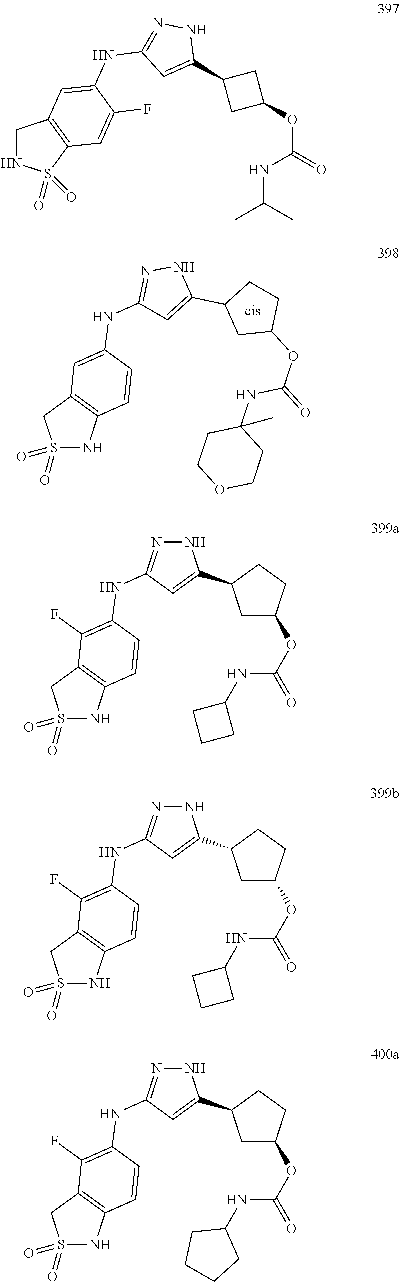 [SMILES: CC(C)NC(=O)O[C@H]1C[C@@H](c2cc(Nc3cc4c(cc3F)S(=O)(=O)NC4)n[nH]2)C1 ; CC1(NC(=O)OC2CCC(c3cc(Nc4ccc5c(c4)CS(=O)(=O)N5)n[nH]3)C2)CCOCC1 ; O=C(NC1CCC1)O[C@@H]1CC[C@H](c2cc(Nc3ccc4c(c3F)CS(=O)(=O)N4)n[nH]2)C1 ; O=C(NC1CCC1)O[C@H]1CC[C@@H](c2cc(Nc3ccc4c(c3F)CS(=O)(=O)N4)n[nH]2)C1 ; O=C(NC1CCCC1)O[C@@H]1CC[C@H](c2cc(Nc3ccc4c(c3F)CS(=O)(=O)N4)n[nH]2)C1] 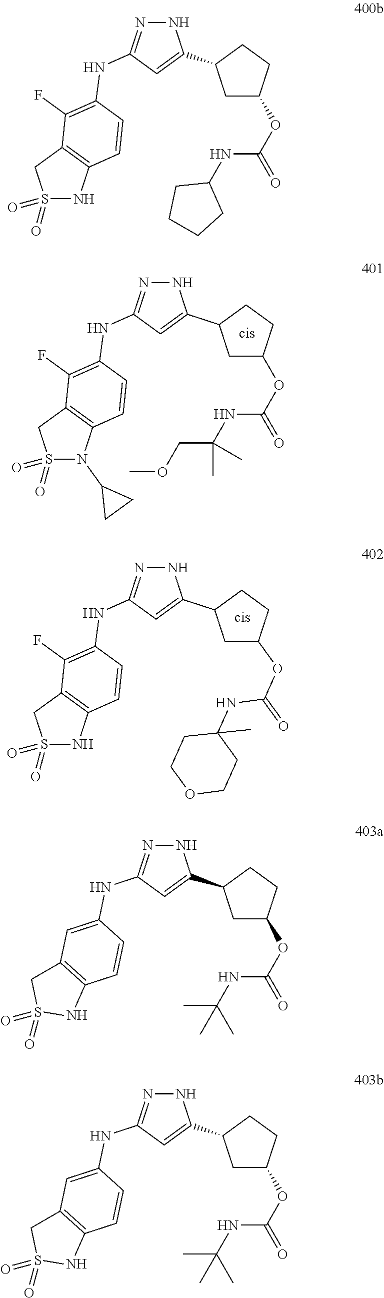 [SMILES: O=C(NC1CCCC1)O[C@H]1CC[C@@H](c2cc(Nc3ccc4c(c3F)CS(=O)(=O)N4)n[nH]2)C1 ; COCC(C)(C)NC(=O)OC1CCC(c2cc(Nc3ccc4c(c3F)CS(=O)(=O)N4C3CC3)n[nH]2)C1 ; CC1(NC(=O)OC2CCC(c3cc(Nc4ccc5c(c4F)CS(=O)(=O)N5)n[nH]3)C2)CCOCC1 ; CC(C)(C)NC(=O)O[C@@H]1CC[C@H](c2cc(Nc3ccc4c(c3)CS(=O)(=O)N4)n[nH]2)C1 ; CC(C)(C)NC(=O)O[C@H]1CC[C@@H](c2cc(Nc3ccc4c(c3)CS(=O)(=O)N4)n[nH]2)C1] 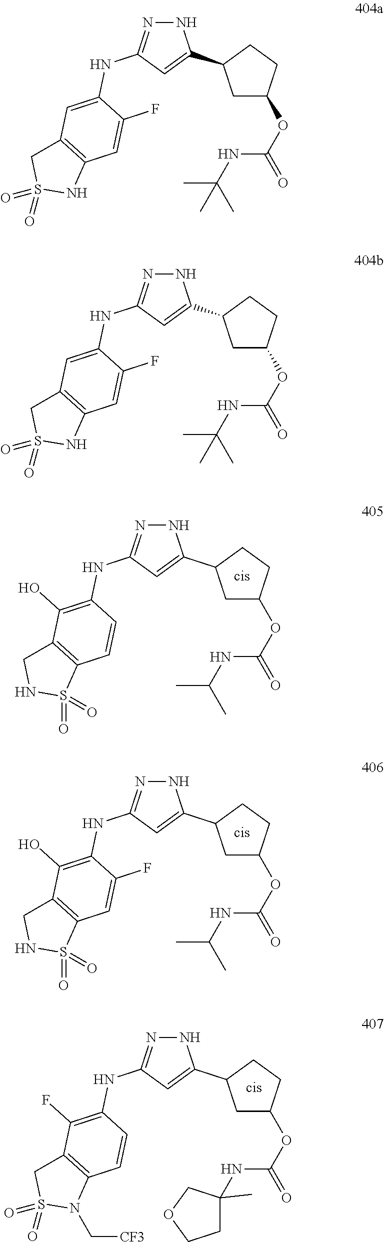 [SMILES: CC(C)(C)NC(=O)O[C@@H]1CC[C@H](c2cc(Nc3cc4c(cc3F)NS(=O)(=O)C4)n[nH]2)C1 ; CC(C)(C)NC(=O)O[C@H]1CC[C@@H](c2cc(Nc3cc4c(cc3F)NS(=O)(=O)C4)n[nH]2)C1 ; CC(C)NC(=O)OC1CCC(c2cc(Nc3ccc4c(c3O)CNS4(=O)=O)n[nH]2)C1 ; CC(C)NC(=O)OC1CCC(c2cc(Nc3c(F)cc4c(c3O)CNS4(=O)=O)n[nH]2)C1 ; CC1(NC(=O)OC2CCC(c3cc(Nc4ccc5c(c4F)CS(=O)(=O)N5CC(F)(F)F)n[nH]3)C2)CCOC1] 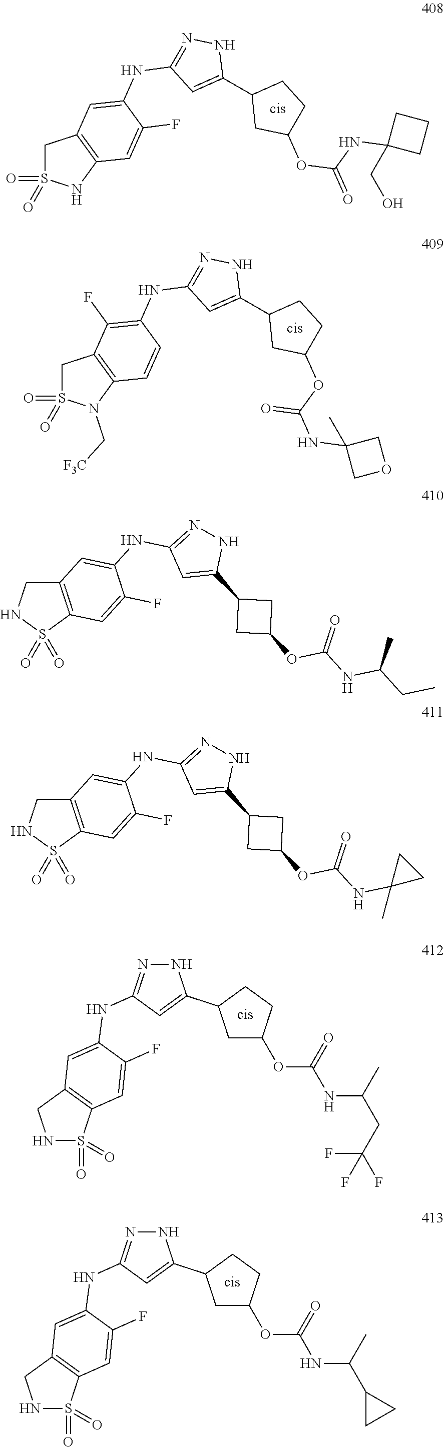 [SMILES: O=C(NC1(CO)CCC1)OC1CCC(c2cc(Nc3cc4c(cc3F)NS(=O)(=O)C4)n[nH]2)C1 ; CC1(NC(=O)OC2CCC(c3cc(Nc4ccc5c(c4F)CS(=O)(=O)N5CC(F)(F)F)n[nH]3)C2)COC1 ; CC[C@H](C)NC(=O)O[C@H]1C[C@@H](c2cc(Nc3cc4c(cc3F)S(=O)(=O)NC4)n[nH]2)C1 ; CC1(NC(=O)O[C@H]2C[C@@H](c3cc(Nc4cc5c(cc4F)S(=O)(=O)NC5)n[nH]3)C2)CC1 ; CC(CC(F)(F)F)NC(=O)OC1CCC(c2cc(Nc3cc4c(cc3F)S(=O)(=O)NC4)n[nH]2)C1 ; CC(NC(=O)OC1CCC(c2cc(Nc3cc4c(cc3F)S(=O)(=O)NC4)n[nH]2)C1)C1CC1] 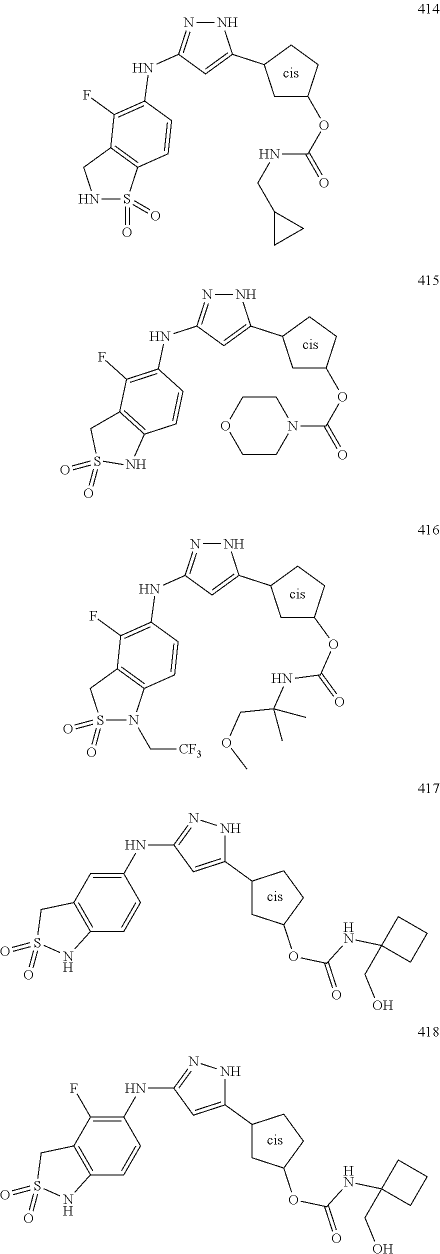 [SMILES: O=C(NCC1CC1)OC1CCC(c2cc(Nc3ccc4c(c3F)CNS4(=O)=O)n[nH]2)C1 ; O=C(OC1CCC(c2cc(Nc3ccc4c(c3F)CS(=O)(=O)N4)n[nH]2)C1)N1CCOCC1 ; COCC(C)(C)NC(=O)OC1CCC(c2cc(Nc3ccc4c(c3F)CS(=O)(=O)N4CC(F)(F)F)n[nH]2)C1 ; O=C(NC1(CO)CCC1)OC1CCC(c2cc(Nc3ccc4c(c3)CS(=O)(=O)N4)n[nH]2)C1 ; O=C(NC1(CO)CCC1)OC1CCC(c2cc(Nc3ccc4c(c3F)CS(=O)(=O)N4)n[nH]2)C1] 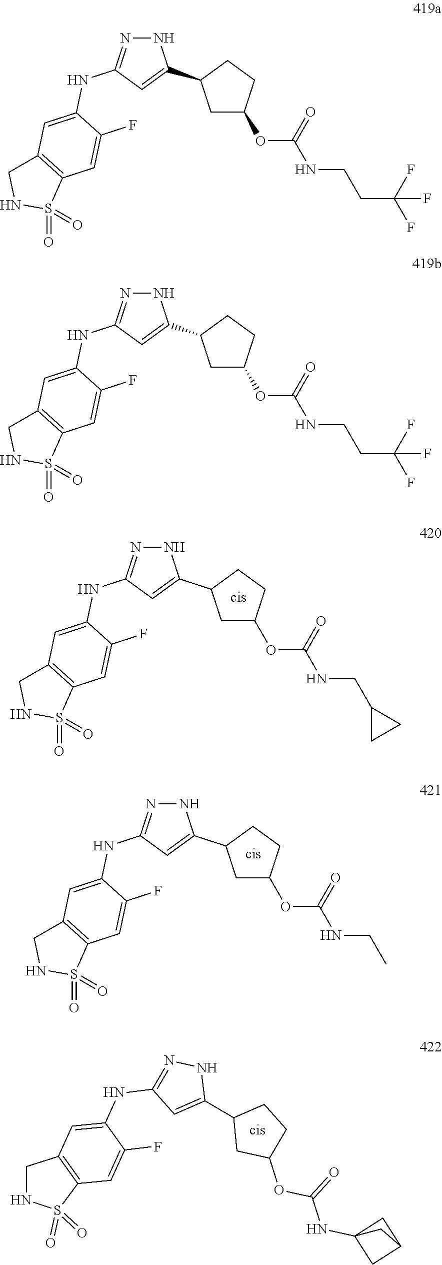 [SMILES: O=C(NCCC(F)(F)F)O[C@@H]1CC[C@H](c2cc(Nc3cc4c(cc3F)S(=O)(=O)NC4)n[nH]2)C1 ; O=C(NCCC(F)(F)F)O[C@H]1CC[C@@H](c2cc(Nc3cc4c(cc3F)S(=O)(=O)NC4)n[nH]2)C1 ; O=C(NCC1CC1)OC1CCC(c2cc(Nc3cc4c(cc3F)S(=O)(=O)NC4)n[nH]2)C1 ; CCNC(=O)OC1CCC(c2cc(Nc3cc4c(cc3F)S(=O)(=O)NC4)n[nH]2)C1 ; O=C(NC12CC(C1)C2)OC1CCC(c2cc(Nc3cc4c(cc3F)S(=O)(=O)NC4)n[nH]2)C1] 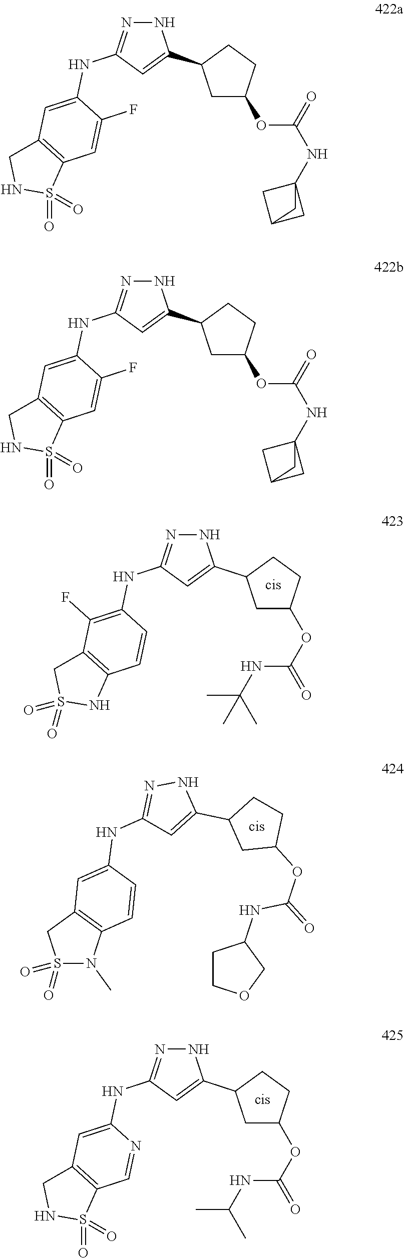 [SMILES: O=C(NC12CC(C1)C2)O[C@@H]1CC[C@H](c2cc(Nc3cc4c(cc3F)S(=O)(=O)NC4)n[nH]2)C1 ; O=C(NC12CC(C1)C2)O[C@@H]1CC[C@H](c2cc(Nc3cc4c(cc3F)S(=O)(=O)NC4)n[nH]2)C1 ; CC(C)(C)NC(=O)OC1CCC(c2cc(Nc3ccc4c(c3F)CS(=O)(=O)N4)n[nH]2)C1 ; CN1c2ccc(Nc3cc(C4CCC(OC(=O)NC5CCOC5)C4)[nH]n3)cc2CS1(=O)=O ; CC(C)NC(=O)OC1CCC(c2cc(Nc3cc4c(cn3)S(=O)(=O)NC4)n[nH]2)C1] 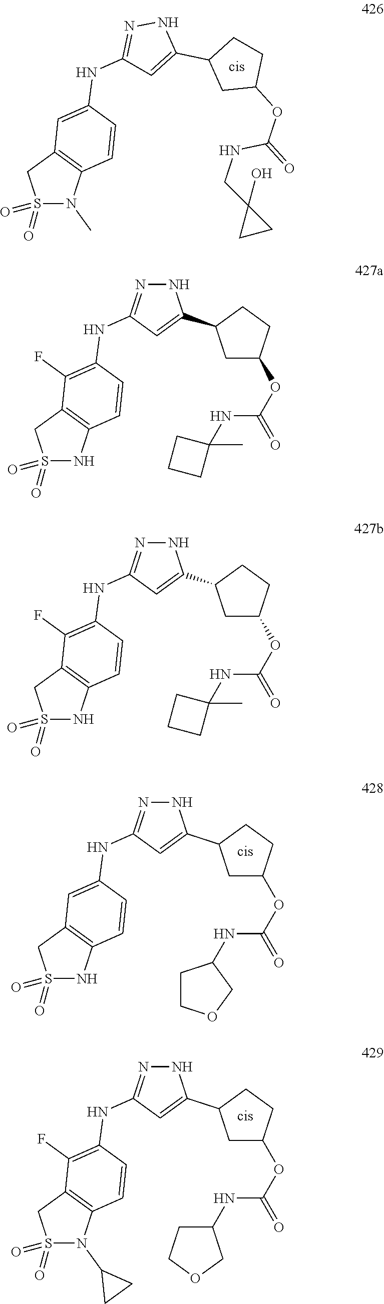 [SMILES: CN1c2ccc(Nc3cc(C4CCC(OC(=O)NCC5(O)CC5)C4)[nH]n3)cc2CS1(=O)=O ; CC1(NC(=O)O[C@@H]2CC[C@H](c3cc(Nc4ccc5c(c4F)CS(=O)(=O)N5)n[nH]3)C2)CCC1 ; CC1(NC(=O)O[C@H]2CC[C@@H](c3cc(Nc4ccc5c(c4F)CS(=O)(=O)N5)n[nH]3)C2)CCC1 ; O=C(NC1CCOC1)OC1CCC(c2cc(Nc3ccc4c(c3)CS(=O)(=O)N4)n[nH]2)C1 ; O=C(NC1CCOC1)OC1CCC(c2cc(Nc3ccc4c(c3F)CS(=O)(=O)N4C3CC3)n[nH]2)C1] 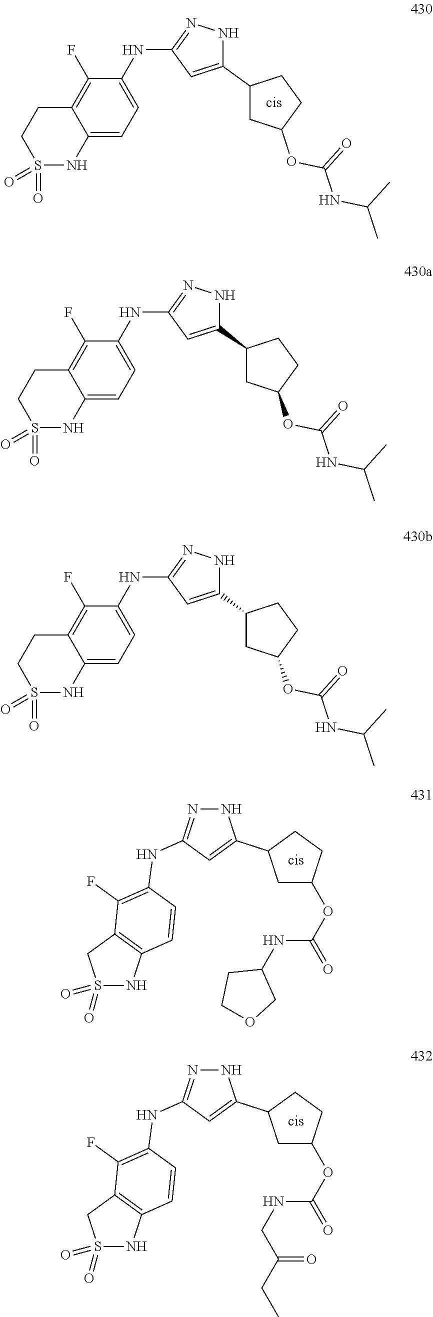 [SMILES: CC(C)NC(=O)OC1CCC(c2cc(Nc3ccc4c(c3F)CCS(=O)(=O)N4)n[nH]2)C1 ; CC(C)NC(=O)O[C@@H]1CC[C@H](c2cc(Nc3ccc4c(c3F)CCS(=O)(=O)N4)n[nH]2)C1 ; CC(C)NC(=O)O[C@H]1CC[C@@H](c2cc(Nc3ccc4c(c3F)CCS(=O)(=O)N4)n[nH]2)C1 ; O=C(NC1CCOC1)OC1CCC(c2cc(Nc3ccc4c(c3F)CS(=O)(=O)N4)n[nH]2)C1 ; CCC(=O)CNC(=O)OC1CCC(c2cc(Nc3ccc4c(c3F)CS(=O)(=O)N4)n[nH]2)C1] 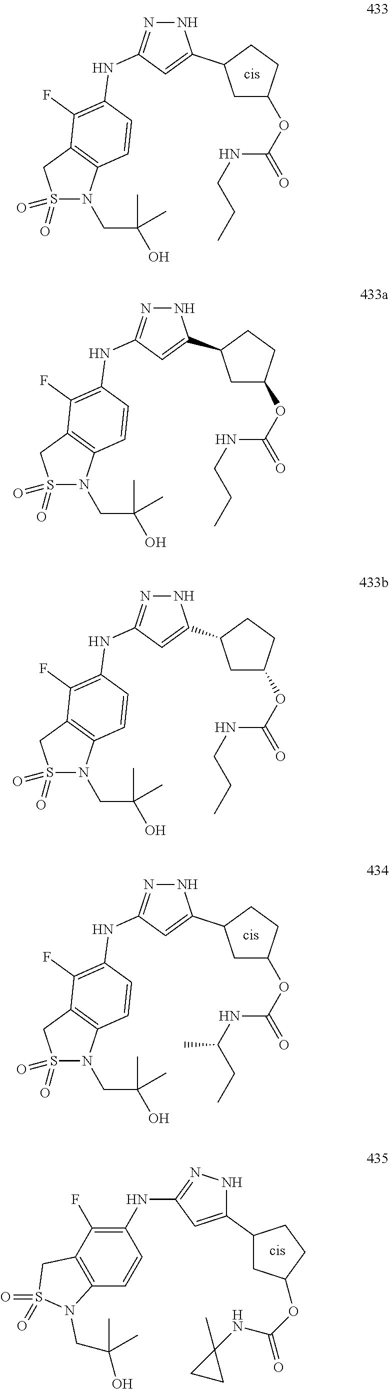 [SMILES: CCCNC(=O)OC1CCC(c2cc(Nc3ccc4c(c3F)CS(=O)(=O)N4CC(C)(C)O)n[nH]2)C1 ; CCCNC(=O)O[C@@H]1CC[C@H](c2cc(Nc3ccc4c(c3F)CS(=O)(=O)N4CC(C)(C)O)n[nH]2)C1 ; CCCNC(=O)O[C@H]1CC[C@@H](c2cc(Nc3ccc4c(c3F)CS(=O)(=O)N4CC(C)(C)O)n[nH]2)C1 ; CC[C@H](C)NC(=O)OC1CCC(c2cc(Nc3ccc4c(c3F)CS(=O)(=O)N4CC(C)(C)O)n[nH]2)C1 ; CC(C)(O)CN1c2ccc(Nc3cc(C4CCC(OC(=O)NC5(C)CC5)C4)[nH]n3)c(F)c2CS1(=O)=O] 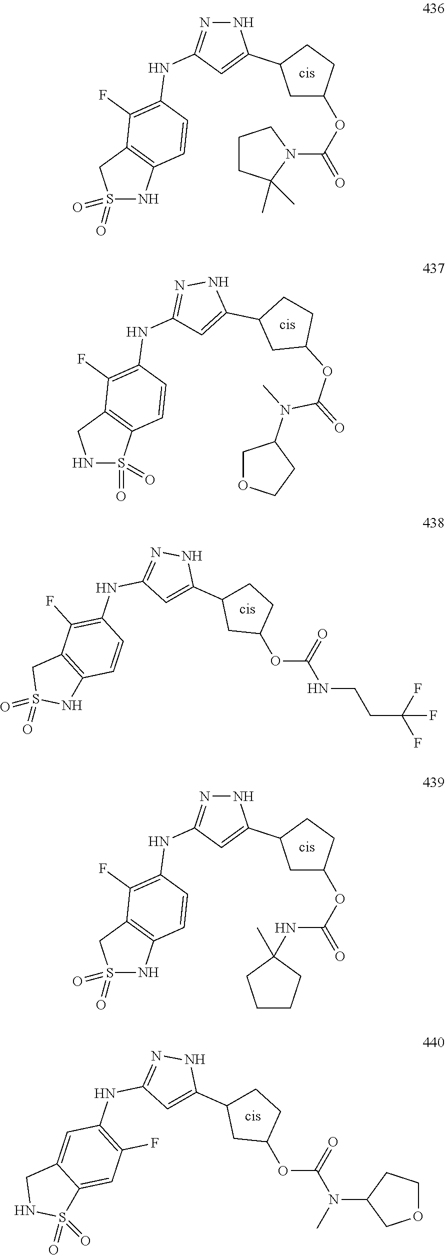 [SMILES: CC1(C)CCCN1C(=O)OC1CCC(c2cc(Nc3ccc4c(c3F)CS(=O)(=O)N4)n[nH]2)C1 ; CN(C(=O)OC1CCC(c2cc(Nc3ccc4c(c3F)CNS4(=O)=O)n[nH]2)C1)C1CCOC1 ; O=C(NCCC(F)(F)F)OC1CCC(c2cc(Nc3ccc4c(c3F)CS(=O)(=O)N4)n[nH]2)C1 ; CC1(NC(=O)OC2CCC(c3cc(Nc4ccc5c(c4F)CS(=O)(=O)N5)n[nH]3)C2)CCCC1 ; CN(C(=O)OC1CCC(c2cc(Nc3cc4c(cc3F)S(=O)(=O)NC4)n[nH]2)C1)C1CCOC1] 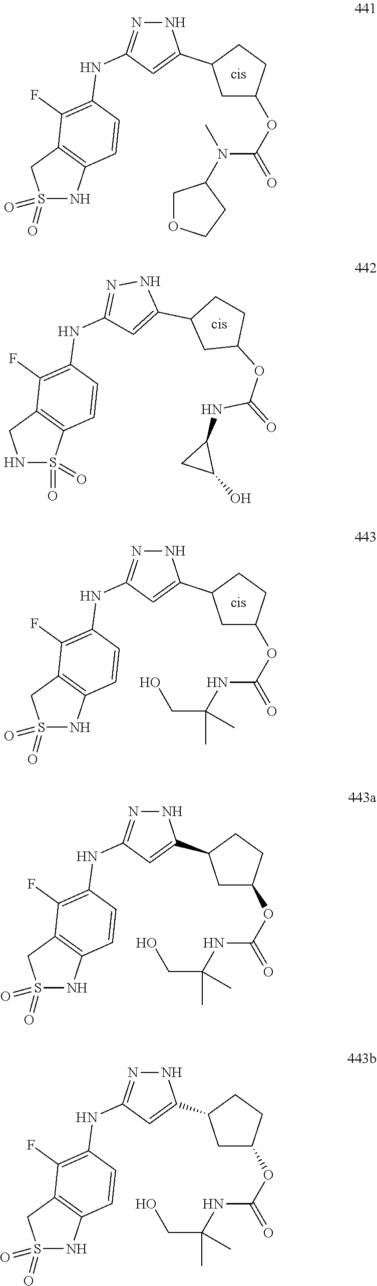 [SMILES: CN(C(=O)OC1CCC(c2cc(Nc3ccc4c(c3F)CS(=O)(=O)N4)n[nH]2)C1)C1CCOC1 ; O=C(N[C@@H]1C[C@H]1O)OC1CCC(c2cc(Nc3ccc4c(c3F)CNS4(=O)=O)n[nH]2)C1 ; CC(C)(CO)NC(=O)OC1CCC(c2cc(Nc3ccc4c(c3F)CS(=O)(=O)N4)n[nH]2)C1 ; CC(C)(CO)NC(=O)O[C@@H]1CC[C@H](c2cc(Nc3ccc4c(c3F)CS(=O)(=O)N4)n[nH]2)C1 ; CC(C)(CO)NC(=O)O[C@H]1CC[C@@H](c2cc(Nc3ccc4c(c3F)CS(=O)(=O)N4)n[nH]2)C1] 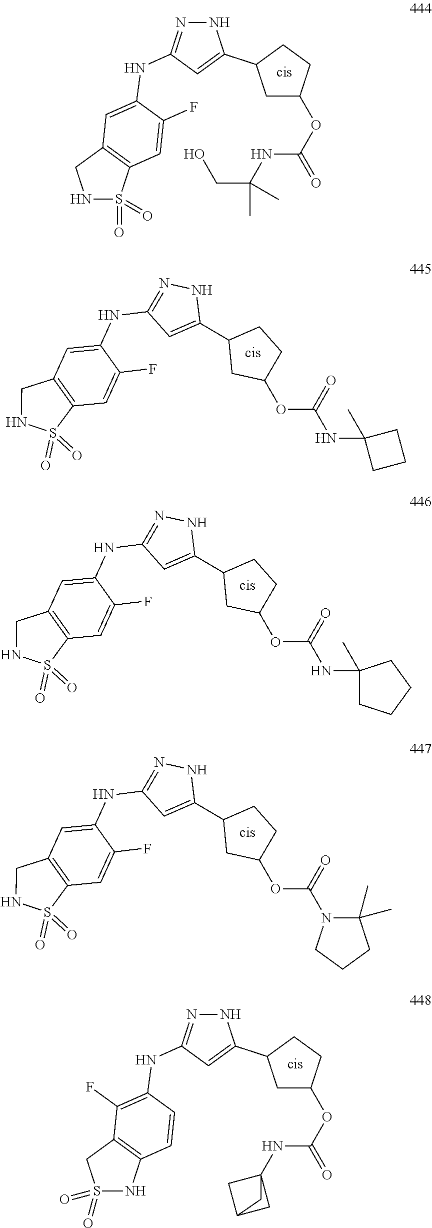 [SMILES: CC(C)(CO)NC(=O)OC1CCC(c2cc(Nc3cc4c(cc3F)S(=O)(=O)NC4)n[nH]2)C1 ; CC1(NC(=O)OC2CCC(c3cc(Nc4cc5c(cc4F)S(=O)(=O)NC5)n[nH]3)C2)CCC1 ; CC1(NC(=O)OC2CCC(c3cc(Nc4cc5c(cc4F)S(=O)(=O)NC5)n[nH]3)C2)CCCC1 ; CC1(C)CCCN1C(=O)OC1CCC(c2cc(Nc3cc4c(cc3F)S(=O)(=O)NC4)n[nH]2)C1 ; O=C(NC12CC(C1)C2)OC1CCC(c2cc(Nc3ccc4c(c3F)CS(=O)(=O)N4)n[nH]2)C1] 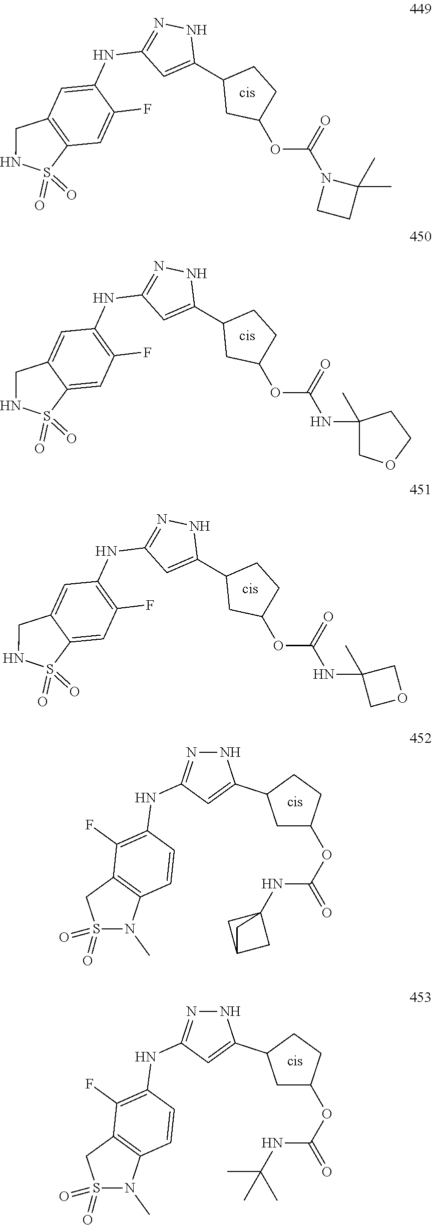 [SMILES: CC1(C)CCN1C(=O)OC1CCC(c2cc(Nc3cc4c(cc3F)S(=O)(=O)NC4)n[nH]2)C1 ; CC1(NC(=O)OC2CCC(c3cc(Nc4cc5c(cc4F)S(=O)(=O)NC5)n[nH]3)C2)CCOC1 ; CC1(NC(=O)OC2CCC(c3cc(Nc4cc5c(cc4F)S(=O)(=O)NC5)n[nH]3)C2)COC1 ; CN1c2ccc(Nc3cc(C4CCC(OC(=O)NC56CC(C5)C6)C4)[nH]n3)c(F)c2CS1(=O)=O ; CN1c2ccc(Nc3cc(C4CCC(OC(=O)NC(C)(C)C)C4)[nH]n3)c(F)c2CS1(=O)=O] 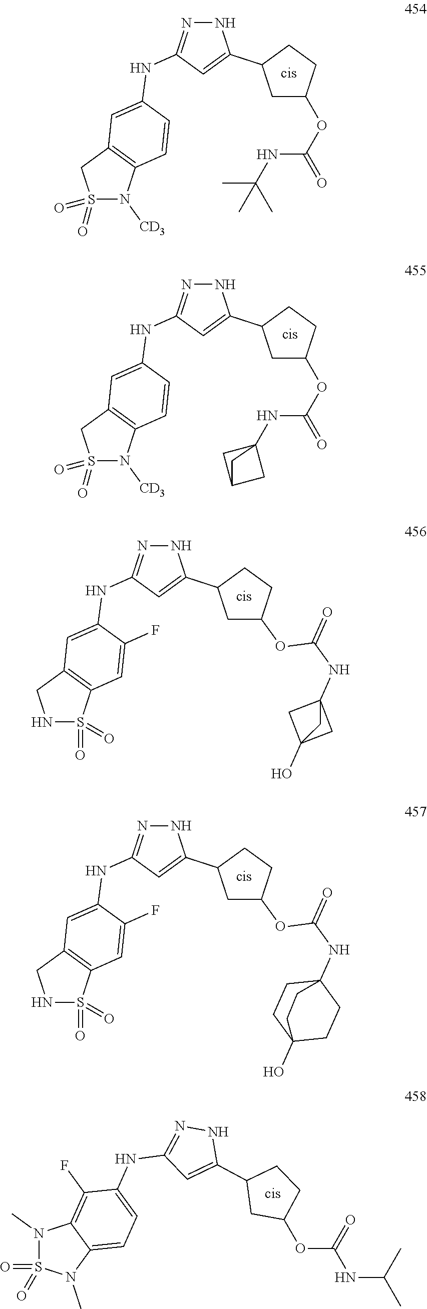 [SMILES: [2H]C([2H])([2H])N1c2ccc(Nc3cc(C4CCC(OC(=O)NC(C)(C)C)C4)[nH]n3)cc2CS1(=O)=O ; [2H]C([2H])([2H])N1c2ccc(Nc3cc(C4CCC(OC(=O)NC56CC(C5)C6)C4)[nH]n3)cc2CS1(=O)=O ; O=C(NC12CC(O)(C1)C2)OC1CCC(c2cc(Nc3cc4c(cc3F)S(=O)(=O)NC4)n[nH]2)C1 ; O=C(NC12CCC(O)(CC1)CC2)OC1CCC(c2cc(Nc3cc4c(cc3F)S(=O)(=O)NC4)n[nH]2)C1 ; CC(C)NC(=O)OC1CCC(c2cc(Nc3ccc4c(c3F)N(C)S(=O)(=O)N4C)n[nH]2)C1] 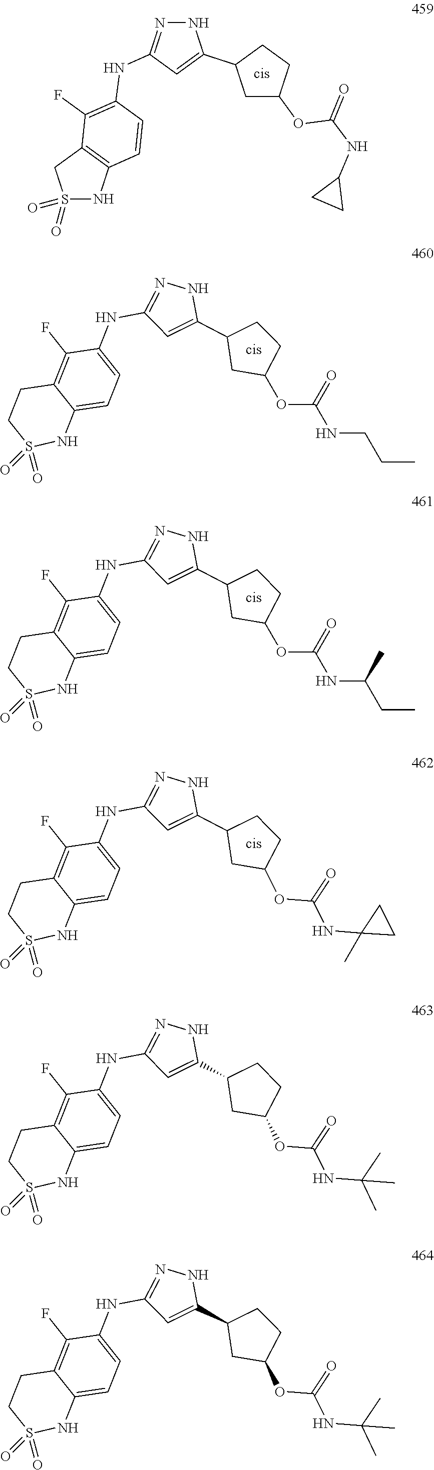 [SMILES: O=C(NC1CC1)OC1CCC(c2cc(Nc3ccc4c(c3F)CS(=O)(=O)N4)n[nH]2)C1 ; CCCNC(=O)OC1CCC(c2cc(Nc3ccc4c(c3F)CCS(=O)(=O)N4)n[nH]2)C1 ; CC[C@H](C)NC(=O)OC1CCC(c2cc(Nc3ccc4c(c3F)CCS(=O)(=O)N4)n[nH]2)C1 ; CC1(NC(=O)OC2CCC(c3cc(Nc4ccc5c(c4F)CCS(=O)(=O)N5)n[nH]3)C2)CC1 ; CC(C)(C)NC(=O)O[C@H]1CC[C@@H](c2cc(Nc3ccc4c(c3F)CCS(=O)(=O)N4)n[nH]2)C1 ; CC(C)(C)NC(=O)O[C@@H]1CC[C@H](c2cc(Nc3ccc4c(c3F)CCS(=O)(=O)N4)n[nH]2)C1] 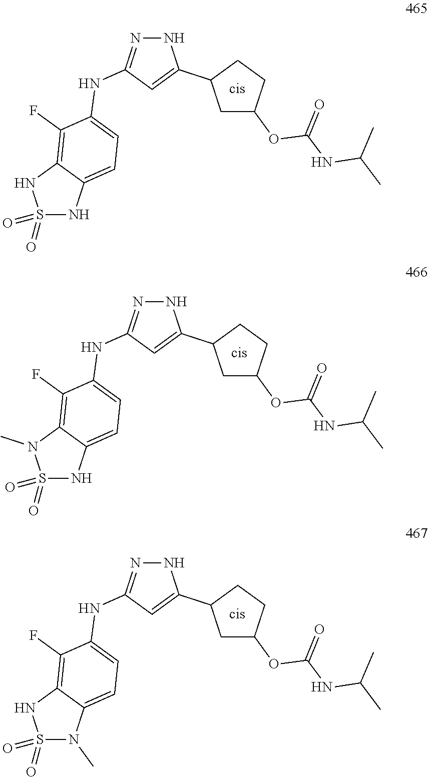
or a N-oxide thereof, or a pharmaceutically acceptable salt thereof, or a stereoisomer thereof, or a tautomer thereof, or a deuterated analog thereof, or a prodrug thereof.

20. A pharmaceutical composition comprising a compound of claim 1, or a pharmaceutically acceptable salt thereof, or a stereoisomer, a tautomer or a prodrug thereof, and at least one pharmaceutically acceptable carrier or excipient.

21. A method of treating cancer, comprising administering to a subject in need thereof a compound of claim 1, or a pharmaceutically acceptable salt, or a stereoisomer, a tautomer or a prodrug thereof.

22. The compound of claim 1, wherein R³ and R^{Q} are each independently selected from hydrogen, methyl, ethyl, propyl, butyl;
R¹ and R² are each independently selected from hydrogen, methyl, ethyl, propyl, n-butyl,

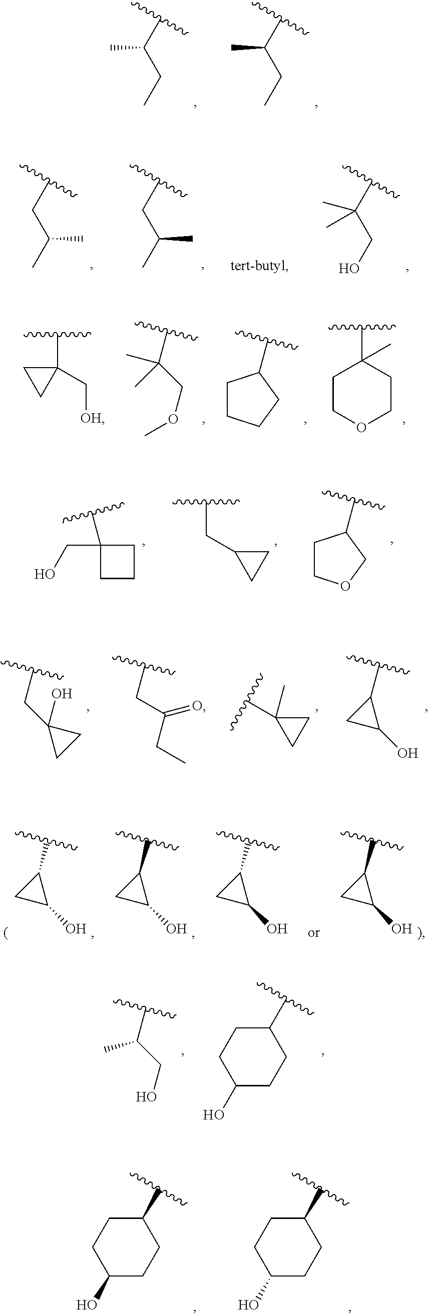

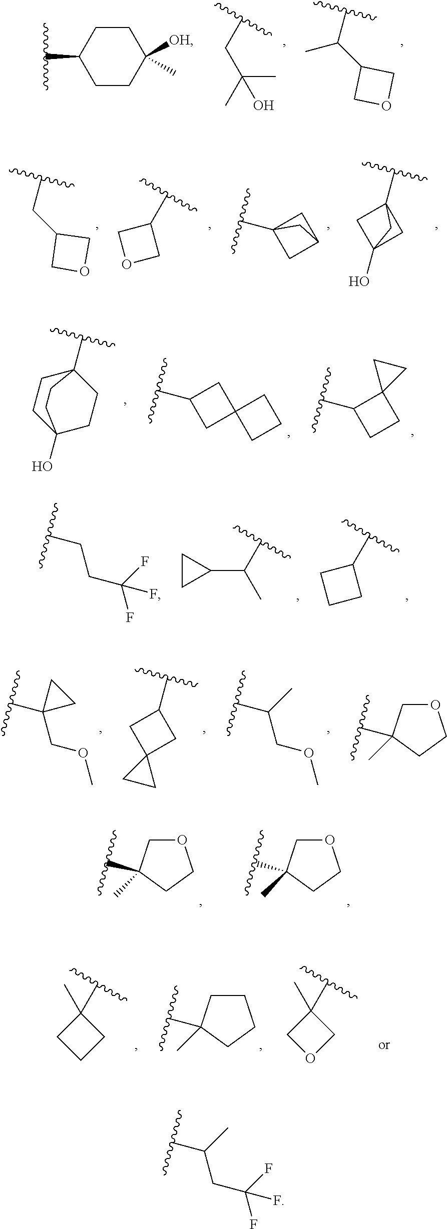

23. The compound of claim 1, wherein, R¹ is independently selected from methyl, ethyl, propyl, n-butyl, 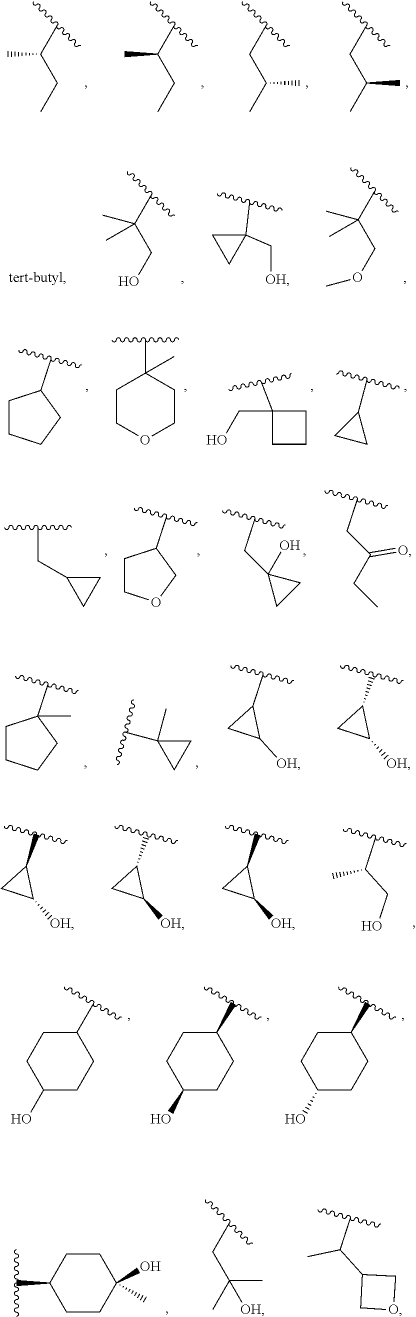 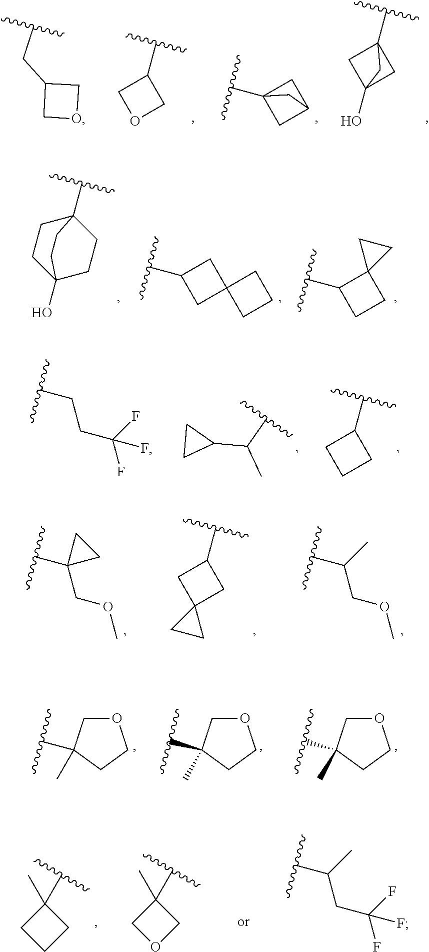
and R² is hydrogen.

24. The compound of claim 1, wherein Z¹ is N and Z² and Z³ are —CR^{Z}; or Z² is N and Z¹ and Z³ are —CR^{Z}; or Z³ is N and Z¹ and Z² are —CR^{Z}.

25. The compound of claim 1, wherein R^{Z}, at each occurrence, is independently selected from hydrogen, —F, —Cl, methyl, ethyl, propyl, butyl, cyclopropyl, cyclobutyl, cyclopentyl, cyclohexyl, —CH₂F, —CHF₂, —CF₃, —CH₂CH₂F, —CH₂OH, —CH₂CHF₂, —CH₂CF₃, —NH₂, —OH, —OCHF₂, methoxy, ethoxy, propoxy, or butoxy.

26. The compound of claim 1, R^{Xa} is each independently selected from hydrogen, methyl, ethyl, propyl, butyl, cyclopropyl, cyclobutyl, cyclopentyl, —C₂H₄OH, —C₂H₄OMe, —C₃H₆OH, —C₃H₆OMe, —CH₂OMe, —C(CH₃)₂OH, 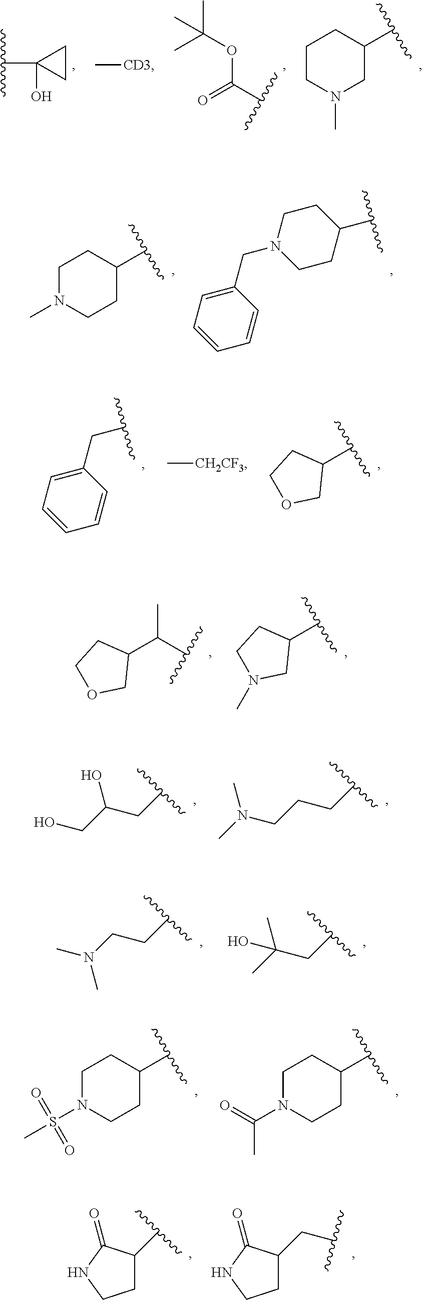 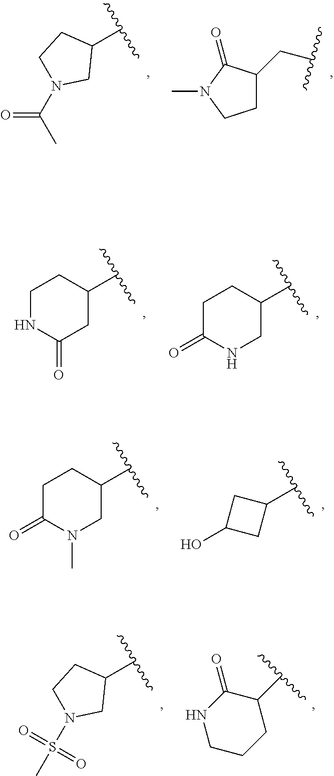 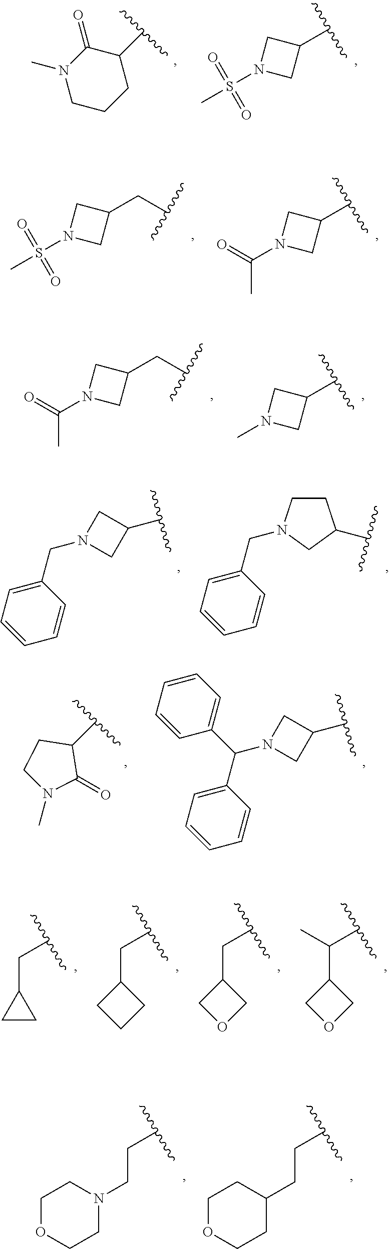 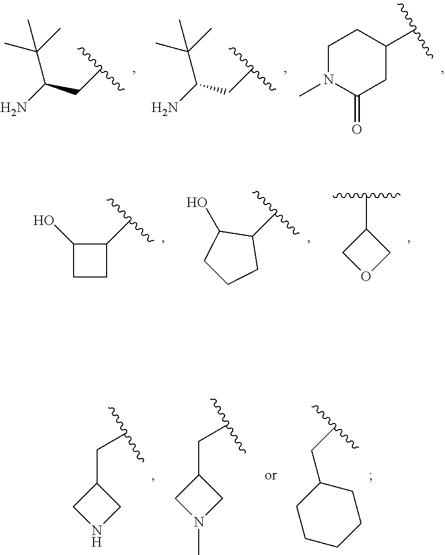
R^{Xb} is independently selected from hydrogen, —F, —Cl, methyl, -CD₃, ethyl, propyl, butyl, cyclopropyl, cyclobutyl, cyclopentyl, cyclohexyl, —CH₂F, —CHF₂, —CF₃, —CH₂CH₂F, —CH₂CHF₂, —CH₂CF₃, oxo (═O), or —OH.
